(19) United States
(12) Patent Application Publication
(10) Pub. No.: US 2024/0383886 A1
(43) Pub. Date: Nov. 21, 2024

(54) Protein Tyrosine Phosphatase Degraders and Methods of Use Thereof

(71) Applicants: CALICO LIFE SCIENCES LLC, South San Francisco, CA (US); ABBVIE INC., North Chicago, IL (US)

(72) Inventors: Gesine Kerstin Veits, Watertown, MA (US); Mark E. Fitzgerald, Watertown, MA (US); Alexander W. Hird, Watertown, MA (US); James A. Henderson, Watertown, MA (US); Harit U. Vora, Watertown, MA (US); Ramzi F. Sweis, North Chicago, IL (US); Michael E. Kort, North Chicago, IL (US); Christopher G. Nasveschuk, Stoneham, MA (US); Martin Duplessis, Somerville, MA (US)

(21) Appl. No.: 17/787,828

(22) PCT Filed: Dec. 18, 2020
(86) PCT No.: PCT/US2020/066243

§ 371 Date: Jun. 21, 2022

Related U.S. Application Data
(60) Provisional application No. 62/952,097, filed on Dec. 20, 2019.
(60) Provisional application No. 62/952,161, filed on Dec. 20, 2019.
(60) Provisional application No. 63/121,721, filed on Dec. 4, 2020.
(60) Provisional application No. 63/125,937, filed on Dec. 15, 2020.

(51) Int. Cl. C07D 417/14; A61K 31/454; A61K 31/4545; A61K 31/496; C07D 471/04

(52) U.S. Cl. CPC C07D 417/14; A61K 31/454; A61K 31/4545; A61K 31/496; C07D 471/04

(57) ABSTRACT

Provided herein are compounds, compositions, and methods useful for degrading protein tyrosine phosphatase, e.g., protein tyrosine phosphatase non-receptor type 2 (PTPN2) and/or protein tyrosine phosphatase non-receptor type 1 (PTPN1), and for treating related diseases favorably responsive to PTPN1 or PTPN2 inhibitor treatment, e.g., a cancer 5 or a metabolic disease.

### CROSS-REFERENCE TO RELATED APPLICATION

This application is an international application and claims the benefit of U.S. Provisional Application No. 62/952,097, filed Dec. 20, 2019; U.S. Provisional Application No. 63/121,721, filed Dec. 4, 2020; U.S. Provisional Application No. 63/125,937, filed Dec. 15, 2020; U.S. Provisional Application No. 62/952,161, filed Dec. 20, 2019. The disclosure of the foregoing applications are hereby incorporated by reference in their entireties.

### BACKGROUND

Cancer immunotherapy regimens targeting immune evasion mechanisms including checkpoint blockade (e.g. PD-1/PD-L1 and CTLA-4 blocking antibodies) have been shown to be effective in treating in a variety of cancers, dramatically improving outcomes in some populations refractory to conventional therapies. However, incomplete clinical responses and the development of intrinsic or acquired resistance will continue to limit the subject populations who could benefit from checkpoint blockade.

Protein tyrosine phosphatase non-receptor type 2 (PTPN2), also known as T cell protein tyrosine phosphatase (TC-PTP), is an intracellular member of the class 1 subfamily of phospho-tyrosine specific phosphatases that control multiple cellular regulatory processes by removing phosphate groups from tyrosine substrates. PTPN2 is ubiquitously expressed, but expression is highest in hematopoietic and placental cells (Mosinger, B. Jr. et al., Proc Natl Acad Sci USA 89:499-503; 1992). In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif (Tillmann U. et al., Mol Cell Biol 14:3030-3040; 1994). The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g. JAK1, JAK3), receptor tyrosine kinases (e.g. INSR, EGFR, CSF1R, PDGFR), transcription factors (e.g. STAT1, STAT3, STAT5a/b), and Src family kinases (e.g. Fyn, Lck). As a critical negative regulator of the JAK-STAT pathway, PTPN2 functions to directly regulate signaling through cytokine receptors, including IFNγ. The PTPN2 catalytic domain shares 74% sequence homology with PTPN1 (also called PTP1B), and shares similar enzymatic kinetics (Romsicki Y. et al., Arch Biochem Biophys 414:40-50; 2003).

Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade (Manguso R. T. et al., Nature 547:413-418; 2017). Loss of Ptpn2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosine phosphatase-1B (PTP1B), has been shown to play a key role in insulin and leptin signaling and is a primary mechanism for down-regulating both the insulin and leptin receptor signaling pathways (Kenner K. A. et al., J Biol Chem 271: 19810-19816, 1996). Animals deficient in PTP1B have improved glucose regulation and lipid profiles and are resistant to weight gain when treated with a high fat diet (Elchebly M. et al., Science 283: 1544-1548, 1999).

One approach to externally impact protein activity is by decreasing levels of a particular protein by targeted protein degradation. Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling. DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection.

Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487); Berndsen et al. (Nat. Struct. Mol. Biol., 2014, 21, 301-307); Deshaies et al. (Ann. Rev. Biochem., 2009, 78, 399-434); Spratt et al. (Biochem. 2014, 458, 421-437); and Wang et al. (Nat. Rev. Cancer., 2014, 14, 233-347).

The first E3 ligase successfully targeted with a small molecule was SCF^{βTrCP}, using a hybrid of the small molecule MetAP2 inhibitor linked to a IκBα phosphopeptide epitope known to bind to the ubiquitin E3 ligase. (Sakamoto et al, PNAS 2001, 98 (15) 8554). Schneekloth et al. describe a degradation agent (PROTAC3) that targets the FK506 binding protein (FKBP12) and shows that both PROTAC2 and PROTAC3 hit their respective targets with green fluorescent protein (GFP) imaging. Schneekloth et al. (Chem Bio Chem 2005, 6, 40-46).

In unrelated parallel research, scientists were investigating thalidomide toxicity, and discovered that cereblon is a thalidomide binding protein. Ito et al. (Science 2010, 327, 1345-1350). Cereblon forms part of an E3 ubiquitin ligase protein complex which interacts with damaged DNA binding protein 1, forming an E3 ubiquitin ligase complex with Cullin 4 and the E2-binding protein ROC1 (also known as RBX1) where it functions as a substrate receptor to select proteins for ubiquitination. The study revealed that thalidomide-cereblon binding in vivo may be responsible for thalidomide teratogenicity. After the discovery that thalidomide binds to the cereblon E3 ubiquitin ligase led to research to investigate incorporating thalidomide and certain derivatives into compounds for the targeted destruction of proteins. See G. Lu et al., (Science, 343, 305-309 (2014)); and J. Kronke et al., (Science, 343, 301-305 (2014)).

While progress has been made in the area of modulation of the UPP for in vivo protein degradation, it would be useful to have additional compounds and approaches to more fully harness the UPP for therapeutic treatments, for example, for the development of targeted PTP1B degraders useful for the treatment of type 2 diabetes, obesity, and metabolic syndrome. It is an object of the present disclosure to provide new compounds, methods, compositions, and methods of manufacture that are useful to degrade selected proteins, e.g., PTP1B, in vivo.

### SUMMARY

The present disclosure is directed, at least in part, to compounds, compositions, and methods that cause degradation of a protein tyrosine phosphatase, e.g., protein tyrosine phosphatase non-receptor type 2 (PTPN2) and/or protein tyrosine phosphatase non-receptor type 1 ((PTPN1), also known as protein tyrosine phosphatase-1B (PTP1B) via the ubiquitin proteasome pathway (UPP). In some embodiments, the compounds described herein comprise a “Targeting Ligand” that binds to a protein tyrosine phosphatase, a “Degron” which binds (e.g., non-covalently) to an E3 Ligase (e.g., the cereblon component) and a linker that covalently links the Targeting Ligand to the Degron.

Some embodiments provide a compound of Formula (I):

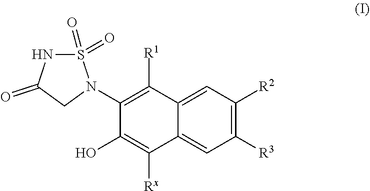

or a pharmaceutically acceptable salt thereof, wherein: R¹; R²; R³; R⁴; R⁵; R⁶; R⁷; R⁸; R⁹; R¹⁰; R^{A}; R^{B}; R^{x}; L; U; V; W; X; Y; Z; Q; p; and q are as defined herein.

Some embodiments provide a pharmaceutical composition comprising the compound of Formula (I), or a pharmaceutically acceptable salt thereof.

Some embodiments provide a method of treating cancer in a subject in need thereof, comprising administering to the subject a therapeutically effective amount of a compound of Formula (I), or a pharmaceutically acceptable salt thereof.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### BRIEF DESCRIPTION OF THE SEQUENCE LISTING

Incorporated herein by reference in its entirety is a Sequence Listing entitled, “45629_0007 WO1_ST25”, comprising SEQ ID NO: 1 through SEQ ID NO: 3, which includes the amino acid sequences disclosed herein. The Sequence listing has been submitted herewith in ASCII text format via EFS. The Sequence Listing was first created on Dec. 19, 2019 and is 7.25 KB in size.

### DETAILED DESCRIPTION

The present disclosure is directed, at least in part, to compounds, compositions, and methods for the inhibition of protein tyrosine phosphatase, e.g., protein tyrosine phosphatase non-receptor type 2 (PTPN2) and/or protein tyrosine phosphatase non-receptor type 1 (PTPN1 or PTP1B).

### Definitions

### Chemical Definitions

Definitions of specific functional groups and chemical terms are described in more detail below: The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C1-C6 alkyl” is intended to encompass, C1, C2, C3, C4, C5, C6, C1-C6, C1-C5, C1-C4, C1-C3, C1-C2, C2-C6, C2-C5, C2-C4, C2-C3, C3-C6, C3-C5, C3-C4, C4-C6, C4-C5, and C5-C6 alkyl.

The following terms are intended to have the meanings presented therewith below and are useful in understanding the description and intended scope of the present disclosure.

“Alkyl” refers to a radical of a straight-chain or branched saturated hydrocarbon group having from 1 to 10 carbon atoms (“C1-C10 alkyl”). In some embodiments, an alkyl group has 1 to 8 carbon atoms (“C1-C8 alkyl”). In some embodiments, an alkyl group has 1 to 6 carbon atoms (“C1-C6 alkyl”). In some embodiments, an alkyl group has 1 to 5 carbon atoms (“C1-C5 alkyl”). In some embodiments, an alkyl group has 1 to 4 carbon atoms (“C1-C4 alkyl”). In some embodiments, an alkyl group has 1 to 3 carbon atoms (“C1-C3 alkyl”). In some embodiments, an alkyl group has 1 to 2 carbon atoms (“C1-C2 alkyl”). In some embodiments, an alkyl group has 1 carbon atom (“C1 alkyl”). In some embodiments, an alkyl group has 2 to 6 carbon atoms (“C2-C6 alkyl”). Examples of C1-C6 alkyl groups include methyl (C1), ethyl (C2), n-propyl (C3), isopropyl (C3), n-butyl (C4), tert-butyl (C4), sec-butyl (C4), iso-butyl (C4), n-pentyl (C5), 3-pentanyl (C5), amyl (C5), neopentyl (C5), 3-methyl-2-butanyl (C5), tertiary amyl (C5), and n-hexyl (C6). Additional examples of alkyl groups include n-heptyl (C7), n-octyl (C8) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C1-C10 alkyl (e.g., —CH₃). In certain embodiments, the alkyl group is substituted C1-C6 alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkenyl” refers to a radical of a straight-chain or branched hydrocarbon group having from 2 to 10 carbon atoms, one or more carbon-carbon double bonds, and no triple bonds (“C2-C10 alkenyl”). In some embodiments, an alkenyl group has 2 to 8 carbon atoms (“C2-C8 alkenyl”). In some embodiments, an alkenyl group has 2 to 6 carbon atoms (“C2-C6 alkenyl”). In some embodiments, an alkenyl group has 2 to 5 carbon atoms (“C2-C5 alkenyl”). In some embodiments, an alkenyl group has 2 to 4 carbon atoms (“C2-C4 alkenyl”). In some embodiments, an alkenyl group has 2 to 3 carbon atoms (“C2-C3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-C4 alkenyl groups include ethenyl (C2), 1-propenyl (C3), 2-propenyl (C3), 1-butenyl (C4), 2-butenyl (C4), butadienyl (C4), and the like. Examples of C2-C6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like. Each instance of an alkenyl group may be independently optionally substituted, e.g., unsubstituted (an “unsubstituted alkenyl”) or substituted (a “substituted alkenyl”) with one or more substituents, e.g., from 1 to 5 substituents, 1 to 3 substituents, or 1 substituent. In certain embodiments, the alkenyl group is unsubstituted C2-C10 alkenyl. In certain embodiments, the alkenyl group is substituted C2-C6 alkenyl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 10 carbon atoms, with those groups having 6 or fewer carbon atoms being preferred in the present disclosure. The term “alkenylene,” by itself or as part of another substituent, means, unless otherwise stated, a divalent radical derived from an alkene. Alkylene groups can be straight chain or branched. An alkylene group may be described as, e.g., a C1-C6 alkylene, which describes an alkylene moiety having between one and six carbon atoms.

“Halo” or “halogen,” independently or as part of another substituent, means a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

“Haloalkyl” refers to an alkyl group as described herein (e.g., a C1-C6 alkyl group) in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl, and 2-fluoroisobutyl.

“Alkoxy” refers to an alkyl group as described herein (e.g., a C1-C6 alkyl group), which is attached to a molecule via oxygen atom. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

“Haloalkoxy” refers to an alkoxy group as described herein (e.g., a C1-C6 alkoxy group), in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkoxy, chloro-difluoroalkoxy, and 2-fluoroisobutoxy.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C6-C14 aryl”). In some embodiments, an aryl group has six ring carbon atoms (“C6 aryl”: e.g., phenyl). In some embodiments, an aryl group has ten ring carbon atoms (“C10 aryl”: e.g., naphthyl such as 1-naphthyl and 2-naphthyl). In some embodiments, an aryl group has fourteen ring carbon atoms (“C14 aryl”: e.g., anthracyl). An aryl group may be described as, e.g., a C6-C10 aryl. Aryl groups include, but are not limited to, phenyl, naphthyl, indenyl, and tetrahydronaphthyl. Each instance of an aryl group may be independently optionally substituted, e.g., unsubstituted (an “unsubstituted aryl”) or substituted (a “substituted aryl”) with one or more substituents. In certain embodiments, the aryl group is unsubstituted C6-C14 aryl. In certain embodiments, the aryl group is substituted C6-C14 aryl.

“Heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl and pyridonyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetravinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted as described herein, and divalent radicals of each heteroaryl example above are non-limiting examples of heteroarylene.

“Aryloxy” refers to an aryl group as described herein (e.g., a C6-C10 aryl group), which is attached to a molecule via oxygen atom. This includes, but it not limited to, groups such as phenoxy and naphthoxy.

“Heteroaryloxy” refers to a heteroaryl group as described herein (e.g., a 5 to 10 membered heteroaryl group), which is attached to a molecule via oxygen atom. This includes, but it not limited to, groups such as pyridinoxy and pyrazinoxy.

“Cycloalkyl” refers to a radical of a saturated or partially unsaturated (i.e., non-aromatic) cyclic hydrocarbon group having from 3 to 10 ring carbon atoms (“C3-C10 cycloalkyl”) and zero heteroatoms in the non-aromatic ring system. In some embodiments, a cycloalkyl group has 3 to 8 ring carbon atoms (“C3-C8cycloalkyl”). In some embodiments, a cycloalkyl group has 3 to 6 ring carbon atoms (“C3-C6 cycloalkyl”). In some embodiments, a cycloalkyl group has 3 to 6 ring carbon atoms (“C3-C6 cycloalkyl”). In some embodiments, a cycloalkyl group has 5 to 10 ring carbon atoms (“C5-C10 cycloalkyl”). A cycloalkyl group may be described as, e.g., a C4-C7-membered cycloalkyl. Exemplary C3-C6 cycloalkyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-C8 cycloalkyl groups include, without limitation, the aforementioned C3-C6 cycloalkyl groups as well as cycloheptyl (C7), cycloheptenyl (C7), cycloheptadienyl (C7), cycloheptatrienyl (C7), cyclooctyl (C8), cyclooctenyl (C8), cubanyl (C8), bicyclo[1.1.1]pentanyl (C5), bicyclo[2.2.2]octanyl (C8), bicyclo[2.1.1]hexanyl (C6), bicyclo[3.1.1]heptanyl (C7), and the like. Exemplary C3-C10 cycloalkyl groups include, without limitation, the aforementioned C3-C8 cycloalkyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the cycloalkyl group is either monocyclic (“monocyclic cycloalkyl”) or contain a fused, bridged, or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, e.g., unsubstituted (an “unsubstituted cycloalkyl”) or substituted (a “substituted cycloalkyl”) with one or more substituents. In certain embodiments, the cycloalkyl group is unsubstituted C3-C10 cycloalkyl. In certain embodiments, the cycloalkyl group is a substituted C3-C10 cycloalkyl.

In some embodiments, “cycloalkyl” is a monocyclic or bicyclic, saturated or partially unsaturated group having from 3 to 10 ring carbon atoms (“C3-C10 cycloalkyl”). In some embodiments, a cycloalkyl group has 3 to 8 ring carbon atoms (“C3-C8 cycloalkyl”). In some embodiments, a cycloalkyl group has 3 to 6 ring carbon atoms (“C3-C6 cycloalkyl”). In some embodiments, a cycloalkyl group has 5 to 6 ring carbon atoms (“C5-C6 cycloalkyl”). In some embodiments, a cycloalkyl group has 5 to 10 ring carbon atoms (“C5-C10 cycloalkyl”). Examples of C5-C6 cycloalkyl groups include cyclopentyl and cyclopentenyl (C5) and cyclohexyl and cyclohexenyl (C6). Examples of C3-C6 cycloalkyl groups include the aforementioned C5-C6 cycloalkyl groups as well as cyclopropyl (C3) and cyclobutyl (C4). Examples of C3-C8 cycloalkyl groups include the aforementioned C3-C6 cycloalkyl groups as well as cycloheptyl (C7) and cyclooctyl (C8). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C3-C10 cycloalkyl. In certain embodiments, the cycloalkyl group is substituted C3-C10 cycloalkyl.

“Heterocyclyl” refers to a radical of a 3- to 12-membered saturated or partially unsaturated (i.e., non-aromatic) ring system having ring carbon atoms and 1 to 4 ring heteroatomic groups, wherein each heteroatomic group is independently selected from nitrogen, oxygen, sulfur, boron, phosphorus, and silicon (“3-12 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon (including oxo groups), nitrogen, oxygen, and sulfur and oxidized forms of sulfur (for example, S, S(O) and S(O)₂), within the moiety. Each instance of heterocyclyl may be independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-12 membered heterocyclyl. In certain embodiments, the heterocyclyl group is substituted 3-12 membered heterocyclyl. In certain embodiments, the heterocyclyl group is substituted 4-6 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, pyrrolidon-2-yl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

A “cycloalkylene” and a “heterocyclylene,” alone or as part of another substituent, mean a divalent radical derived from a cycloalkyl and heterocyclyl, respectively. The examples above may be substituted or unsubstituted as described herein, and divalent radicals of each heterocyclyl example above are non-limiting examples of heterocyclylene and divalent radicals of each cycloalkyl example above are non-limiting examples of cycloalkylene.

“Cycloalkoxy” refers to a cycloalkyl group as described herein (e.g., a C3-C6 cycloalkyl group), which is attached to a molecule via oxygen atom. This includes, but it not limited to, groups such as cyclopropoxy, cyclobutoxy, cyclopentoxy, and cyclohexoxy.

“Heterocyclyloxy” refers to a heterocyclyl group as described herein (e.g., a 4 to 8 membered heterocyclyl group), which is attached to a molecule via oxygen atom. This includes, but it not limited to, groups such as azetidinyloxy, oxetanyloxy, piperidinyloxy, and piperazinyloxy.

“Halocycloalkoxy” refers to a cycloalkoxy group as described herein (e.g., a C3-C6 cycloalkoxy group), in which one or more of the hydrogen atoms are replaced by a halogen (e.g., mono-halocycloalkoxy, di-halocycloalkoxy, tri-halocycloalkoxy, and tetra-halocycloalkoxy). Such groups include but are not limited to, fluorocyclobutoxy, difluorocyclopentoxy, tetrafluorocyclobutoxy, chloro-fluorocycloalkoxy, chloro-difluorocycloalkoxy, and difluorocyclohexoxy.

“Amino” refers to the radical —NH₂.

“Cyano” refers to the radical —CN.

“Hydroxy” or “hydroxyl” refers to the radical —OH.

“Oxo” refers to a ═O) group.

In some embodiments one or more of the nitrogen atoms of a disclosed compound if present are oxidized to the corresponding N-oxide.

As used herein, when a ring is described as being “partially unsaturated”, it means the ring has one or more double or triple bonds between constituent ring atoms, provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

The term “pharmaceutically acceptable salts” is meant to include salts that are prepared with relatively nontoxic acids or bases, depending on the particular substituents found on the compounds described herein.

Certain compounds described herein can exist in unsolvated forms as well as solvated forms, including hydrated forms. In general, the solvated forms are equivalent to unsolvated forms and are encompassed within the scope of the present disclosure.

Certain compounds described herein possess asymmetric carbon atoms (optical or chiral centers) or double bonds: the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds described herein do not include those which are known in art to be too unstable to synthesize and/or isolate. The present disclosure includes compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer. An example of a tautomeric forms includes the following example:

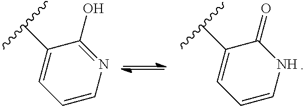 [SMILES: *c1cccnc1O ; *c1ccc[nH]c1=O]

It will be apparent to one skilled in the art that certain compounds of this disclosure may exist in tautomeric forms, all such tautomeric forms of the compounds being within the scope of the disclosure.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as additional agents, e.g., therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure. For example, in some embodiments, one or more C—H groups in the naphthyl ring shown in Formula (I) are replaced with C-D groups.

In the compounds described herein, it is understood that the linker group L does not include compounds, for example, where U and V; V and W; or U, V, and W; are all heteroatoms (e.g., —O—).

“Treating” or “treatment” refers to reducing the symptoms or arresting or inhibiting further development of the disease (in whole or in part). “Treating” or “treatment” includes any effect, e.g., lessening, reducing, modulating, or eliminating, that results in the improvement of the disease and the like. For example, certain methods herein treat cancer by decreasing or reducing the occurrence, growth, metastasis, or progression of cancer or decreasing a symptom of cancer.

An “effective amount” is an amount sufficient to accomplish a stated purpose (e.g. achieve the effect for which it is administered, treat a disease, reduce enzyme activity, increase enzyme activity, or reduce one or more symptoms of a disease). An example of an “effective amount” is an amount sufficient to contribute to the treatment, prevention, or reduction of a symptom or symptoms of a disease, which could also be referred to as a “therapeutically effective amount.” A “prophylactically effective amount” of a drug is an amount of a drug that, when administered to a subject, will have the intended prophylactic effect, e.g., preventing or delaying the onset (or reoccurrence) of a disease, or reducing the likelihood of the onset (or reoccurrence) of a disease or its symptoms.

A “reduction” of a symptom or symptoms means decreasing of the severity or frequency of the symptom(s), or the complete elimination of the symptom(s).

“Contacting” refers to the process of allowing at least two distinct species to become sufficiently proximal to react, interact, and/or physically touch. It should be appreciated, however, that the resulting reaction product can be produced directly from a reaction between the added reagents or from an intermediate from one or more of the added reagents which can be produced in the reaction mixture. The term “contacting” includes allowing two species to react, interact, and/or physically touch, wherein the two species may be a compound as described herein and a protein or enzyme, e.g., a protein tyrosine phosphatase, e.g., protein tyrosine phosphatase non-receptor type 2 (PTPN2) or protein tyrosine phosphatase non-receptor type 1 (PTP1B).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction in the progression of a disease and/or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a protein tyrosine phosphatase, e.g., protein tyrosine phosphatase non-receptor type 2 (PTPN2) or protein tyrosine phosphatase non-receptor type 1 (PTP1B). Thus, inhibition may include, at least in part, partially or totally decreasing stimulation, decreasing or reducing activation, or inactivating, desensitizing, or down-regulating signal transduction or enzymatic activity or the amount of a protein tyrosine phosphatase, e.g., protein tyrosine phosphatase non-receptor type 2 (PTPN2) or protein tyrosine phosphatase non-receptor type 1 (PTP1B).

A “subject,” as used herein, refers to a living organism suffering from or prone to a disease that can be treated by administration of a compound or pharmaceutical composition, as provided herein. Non-limiting examples include mammals such as humans. In some embodiments, a subject is human. In some embodiments, a subject is a new born human. In some embodiments, a subject is an elderly human. In some embodiments, the subject is a pediatric subject (e.g., a subject 21 years of age or less).

“Disease” refers to a state of being or health status of a subject or subject capable of being treated with a compound, pharmaceutical composition, or method provided herein. In some embodiments, the compounds and methods described herein comprise reduction or elimination of one or more symptoms of the disease, e.g., through administration of a compound described herein, a pharmaceutically acceptable salt thereof, or a pharmaceutical composition comprising a compound described herein, or a pharmaceutically acceptable salt thereof.

The term “PTPN2” as used herein refers to protein tyrosine phosphatase non-receptor type 2.

The term “PTPN1” refers to protein tyrosine phosphatase non-receptor type 1 (PTPN1), also known as protein tyrosine phosphatase-1B (PTP1B),

### Compounds

Some embodiments provide a compound of Formula (I):

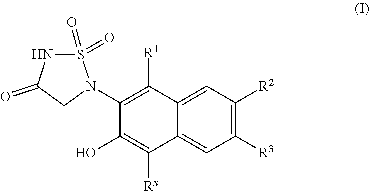

or a pharmaceutically acceptable salt thereof, wherein:
R¹ is hydrogen or halogen;
R² is hydrogen, halogen, C1-C3 alkoxy, C3-C6 cycloalkoxy, C1-C3 haloalkoxy, C3-C5 halocycloalkoxy, C1-C3 alkyl, C1-C3 haloalkyl, C3-C6 cycloalkyl, or -L-Z;
R³ is hydrogen, halogen, C1-C3 alkoxy, C3-C5 cycloalkoxy, C1-C3 haloalkoxy, C3-C5 halocycloalkoxy, C1-C3 alkyl, C1-C3 haloalkyl, C3-C5 cycloalkyl, or -L-Z;
wherein one of R² and R³ is -L-Z and the other of R² and R³ is not-L-Z;
R^{x} is hydrogen or halogen;
L is —U—V—W—X—Y—;
U is a bond, —(NR⁴)—, —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—,
—OR⁵—, —R⁵O—, —NR+R⁵—, —R⁵NR⁴—, or —(NR⁴)(C═O)(NR⁴)—;
each R⁴ is independently a hydrogen, C1-C6 alkyl, or C3-C5 cycloalkyl;
R⁵ is C1-C3 alkylene, C3-C7 cycloalkylene, or 4-12 membered heterocyclylene:
V is a bond, —(NR⁴)—, —O—, C1-C6 alkylene, C2-C6 alkenylene, —(C═O)NR⁴—, —(NR⁴)R⁵—, —(NR⁴)(C═O)—, —NH(C═O)NH—, —OR⁵—, —R⁵O—, 4-10-membered heterocyclylene, 5-10 membered heteroarylene, C6-C10 arylene, or C3-C6 cycloalkylene;
W is a bond, C1-C3 alkylene optionally substituted with hydroxyl, C3-C6 cycloalkylene, 4-12 membered heterocyclylene, —O—, —(NR⁴)—, —R⁵(NR⁴)—, —(NR⁴)R⁵—, —(NR⁴)(C═O)—, —R⁵(NR⁴)(C═O)—, —(C═O)(NR⁴)R⁵—, —R⁵(C═O)(NR⁴)—, —(C═O)(NR⁴)—, —R⁵(C═O)—, —(C═O)R⁵—, —(C═O)—, —(S═O)—, or —S(O₂)—;
X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—;
Y is R⁶, R⁶(CR^{A}R^{B})ₚ-Q-, or -Q-(CR^{A}R^{B})ₚR⁶—;
Q is selected from the group consisting of —(NR⁴)—, —O—, and —(CR^{A}R^{B})ₚ—;
p is 0, 1, 2, or 3;
R⁶ is C1-C3 alkylene, C3-C7 cycloalkylene, 4-12 membered heterocyclylene, C6-C10 arylene, or 5-10 membered heteroarylene;
wherein the heterocyclylene, heteroarylene, arylene, and cycloalkylene groups of U, V, W, X, and R⁶ are each optionally substituted with 1-3 substituents independently selected from fluoro, hydroxyl, C1-C6 alkoxy, and C1-C6 alkyl;
each R^{A} and R^{B} is independently hydrogen, fluoro, or C1-C6 alkyl; or
R^{A} and R^{B}, together with the carbon atom to which they are attached, come together to form a C3-C4 cycloalkyl; or
R^{A} and R^{B} combine to form oxo;
Z is selected from the group consisting of

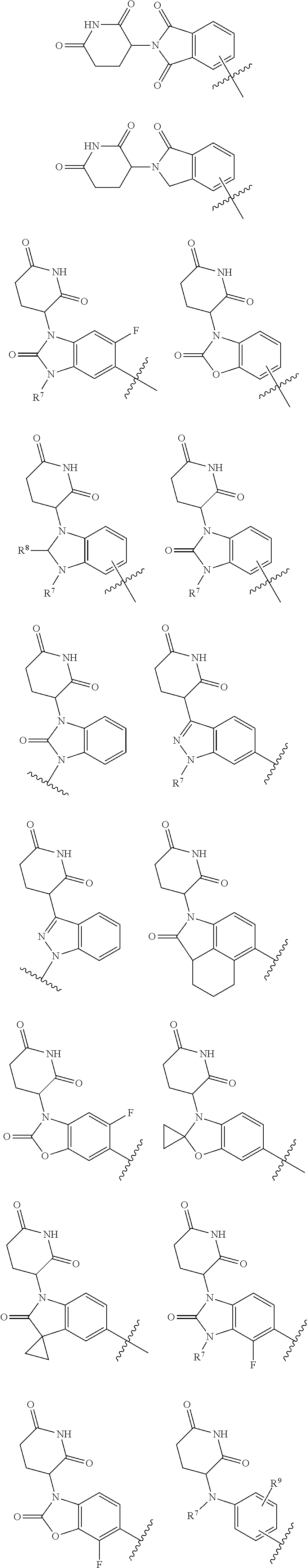

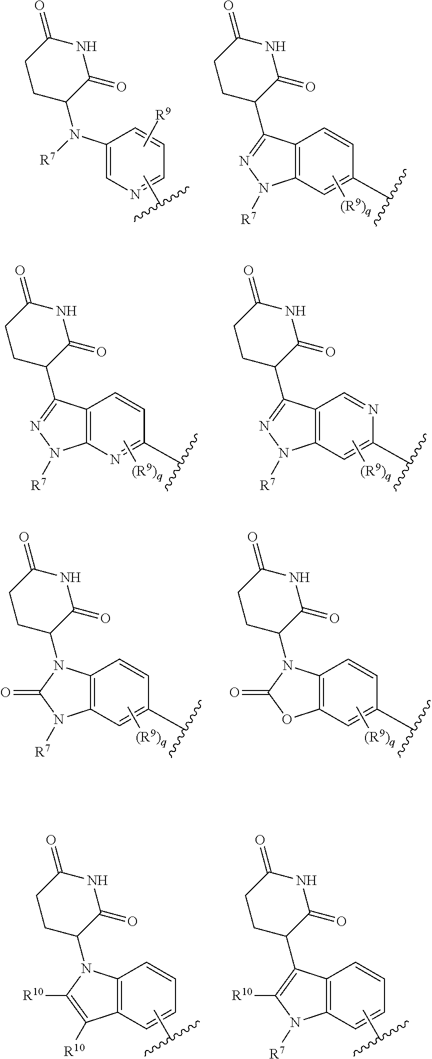

R⁷ is hydrogen, C1-C6 alkyl optionally substituted with one group selected from hydroxyl, cyano and C1-C6 alkoxy, C1-C6 haloalkyl, C3-C6 cycloalkyl, 4-6 membered heterocyclyl, —(CR^{A}R^{B})(4-12 membered heterocyclyl), or —(CR^{A}R^{B})(C3-C6 cycloalkyl);
R⁸ is hydrogen or C1-C6 alkyl;
each R⁹ is hydrogen, halogen, cyano, C1-C6 alkyl, C1-C6 haloalkyl, C1-C6 alkoxy, C1-C5 cycloalkoxy, 5-10 membered heteroaryloxy, or phenoxy; q is 0, 1, or 2; and
each R¹⁰ is independently hydrogen, halogen, cyano, C1-C6 alkyl, C3-C6 cycloalkyl, or C1-C6 haloalkyl.

In some embodiments, L is —U—V—W—X—Y—, wherein —Y— is, for example, the point of connection to Z; and wherein —U— is the point of connection to the remainder of Formula (I) (e.g., the naphthyl ring shown in Formula (I)).

In some embodiments of a compound of Formula (I), R¹ is halogen. In some embodiments of a compound of Formula (I), R¹ is —F. In some embodiments of a compound of Formula (I), R¹ is —Cl. In some embodiments of a compound of Formula (I), R¹ is hydrogen.

In some embodiments of a compound of Formula (I), R^{x} is halogen. In some embodiments of a compound of Formula (I), R^{x} is —F or —Cl. In some embodiments of a compound of Formula (I), R^{x} is hydrogen.

In some embodiments of a compound of Formula (I), R² is -L-Z.

In some embodiments of a compound of Formula (I), R³ is hydrogen. In some embodiments of a compound of Formula (I), R³ is halogen. In some embodiments of a compound of Formula (I), R³ is C1-C3 alkoxy or C1-C3 haloalkoxy. In some embodiments of a compound of Formula (I), R³ is C3-C5 cycloalkoxy or C3-C5 halocycloalkoxy. In some embodiments of a compound of Formula (I), R³ is C1-C3 alkyl or C3-C5 cycloalkyl. In some embodiments of a compound of Formula (I), R³ is C1-C3 haloalkyl.

In some embodiments of a compound of Formula (I), R² is -L-Z and R³ is hydrogen. In some embodiments of a compound of Formula (I), R² is -L-Z and R³ is halogen. In some embodiments of a compound of Formula (I), R² is -L-Z and R³ is C1-C3 alkoxy or C1-C3 haloalkoxy. In some embodiments of a compound of Formula (I), R² is -L-Z and R³ is C3-C5 cycloalkoxy or C3-C5 halocycloalkoxy. In some embodiments of a compound of Formula (I), R² is -L-Z and R³ is C1-C3 alkyl or C3-C5 cycloalkyl.

In some embodiments of a compound of Formula (I), R³ is -L-Z.

In some embodiments of a compound of Formula (I), R² is hydrogen. In some embodiments of a compound of Formula (I), R² is halogen. In some embodiments of a compound of Formula (I), R² is C1-C3 alkoxy or C1-C3 haloalkoxy. In some embodiments of a compound of Formula (I), R² is C3-C5 cycloalkoxy or C3-C5 halocycloalkoxy. In some embodiments of a compound of Formula (I), R² is C1-C3 alkyl or C3-C5 cycloalkyl. In some embodiments of a compound of Formula (I), R² is C1-C3 haloalkyl.

In some embodiments of a compound of Formula (I), R³ is -L-Z and R² is hydrogen. In some embodiments of a compound of Formula (I), R³ is -L-Z and R² is halogen. In some embodiments of a compound of Formula (I), R³ is -L-Z and R² is C1-C3 alkoxy or C1-C3 haloalkoxy. In some embodiments of a compound of Formula (I), R³ is -L-Z and R² is C3-C5 cycloalkoxy or C3-C5 halocycloalkoxy. In some embodiments of a compound of Formula (I), R³ is -L-Z and R² is C1-C3 alkyl or C3-C5 cycloalkyl.

In some embodiments of a compound of Formula (I), R¹ is —F; and R^{x} is hydrogen, —F, or —Cl. In some embodiments of a compound of Formula (I), R¹ is —F; R^{x} is hydrogen; R² is -L-Z; and R³ is hydrogen. In some embodiments of a compound of Formula (I), R¹ is —F; R^{x} is hydrogen; R² is hydrogen; and R³ is -L-Z.

In some embodiments, U is a bond, —(NR⁴)—, —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—, —OR⁵—, —R⁵O—, —NR+R⁵—, —R⁵NR⁴—, or —(NR⁴)(C═O)(NR⁴)—. In some embodiments, U is —(NR⁴)—, —NR+R⁵—, or —R⁵NR⁴—. In some embodiments, U is —(NR⁴)—. In some embodiments, R⁴ is hydrogen. In some embodiments, R⁴ is C1-C6 alkyl. In some embodiments, U is —O—, —OR⁵—, or —R⁵O—. In some embodiments, U is —O—. In some embodiments, U is —NR⁴(C═O)—, —(C═O)NR⁴—, or —(NR⁴)(C═O)(NR⁴)—. In some embodiments, wherein U is —NR⁴(C═O)—. In some embodiments, each R⁴ within U is independently hydrogen or C1-C6 alkyl. In some embodiments, each R⁴ within U is hydrogen.

In some embodiments, wherein U is C1-C3 alkylene, C2-C3 alkenylene, or C2-C3 alkynylene. In some embodiments, U is C2-C3 alkenylene. In some embodiments, U is C2-C3 alkynylene. In some embodiments, U is C3-C6 cycloalkylene, 4-10 membered heterocyclylene, or 5-10 membered heteroarylene; each optionally substituted with 1-3 substituents independently selected from fluoro, hydroxyl, C1-C6 alkoxy, and C1-C6 alkyl. In some embodiments, U is a bond.

In some embodiments, V is a bond, —(NR⁴)—, —O—, C1-C6 alkylene, C2-C6 alkenylene, —(C═O)NR⁴—, —(NR⁴)R⁵—, —(NR⁴)(C═O)—, —NH(C═O)NH—, —OR⁵—, —R⁵O—, 4-10-membered heterocyclylene, 5-10 membered heteroarylene, C6-C10 arylene, or C3-C6 cycloalkylene. In some embodiments, V is C1-C6 alkylene or C2-C6 alkenylene. In some embodiments, V is C1-C6 alkylene. In some embodiments, V is C1-C3 alkylene. In some embodiments, V is methylene or ethylene.

In some embodiments, V is 4-10-membered heterocyclylene, 5-10 membered heteroarylene, C6-C10 arylene, or C3-C6 cycloalkylene; each optionally substituted with 1-3 substituents independently selected from fluoro, hydroxyl, C1-C6 alkoxy, and C1-C6 alkyl. In some embodiments, V is 4-10 membered heterocyclylene, 5-10 membered heteroarylene, C6-C10 arylene, or C3-C6 cycloalkylene; each substituted with 1-3 substituents independently selected from fluoro, hydroxyl, C1-C6 alkoxy, and C1-C6 alkyl. In some embodiments, V is 4-10 membered heterocyclylene, 5-10 membered heteroarylene, C6-C10 arylene, or C3-C6 cycloalkylene.

In some embodiments, V is 4-10-membered heterocyclylene. In some embodiments, V is 4-6-membered heterocyclylene. In some embodiments, V is selected from the group consisting of:

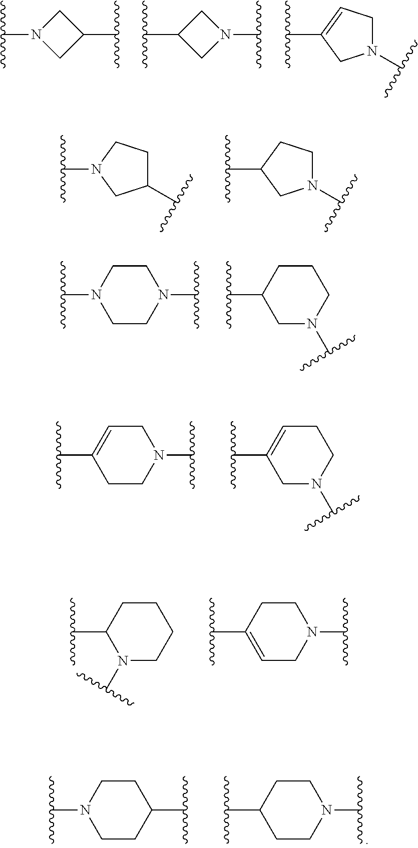

In some embodiments, V is 5-10 membered heteroarylene. In some embodiments, V is 5-6 membered heteroarylene. In some embodiments, V is selected from the group consisting of:

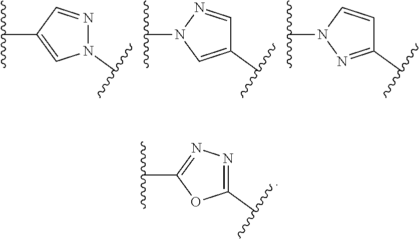 [SMILES: *c1cnn(*)c1 ; *c1cnn(*)c1 ; *c1ccn(*)n1 ; *c1nnc(*)o1]

In some embodiments, V is a C6-C10 arylene. In some embodiments, V is phenyl. In some embodiments, V is naphthyl.

In some embodiments, V is C3-C6 cycloalkylene. In some embodiments, V is selected from the group consisting of cyclobutylene, cyclopentylene, and cyclohexylene.

In some embodiments, V is —(C═O)NR⁴—, —(NR⁴)R⁵—, —(NR⁴)(C═O)—, or —NH(C═O)NH—. In some embodiments, V is —(NR⁴)— or —(NR⁴)R⁵—. In some embodiments, V is —O—, —OR⁵—, or —R⁵O—. In some embodiments, V is a bond.

In some embodiments, W is a bond, C1-C3 alkylene optionally substituted with hydroxyl, C3-C6 cycloalkylene, 4-12 membered heterocyclylene, —O—, —(NR⁴)—, —R⁵(NR⁴)—, —(NR⁴)R⁵—, —(NR⁴)(C═O)—, —R⁵(NR⁴)(C═O)—, —(C═O)(NR⁴)R⁵—, —R⁵(C═O)(NR⁴)—, —(C═O)(NR⁴)—, —R⁵(C═O)—, —(C═O)R⁵—, —(C═O)—, —(S═O)—, or —S(O₂)—.

In some embodiments, W is a bond. In some embodiments, W is C1-C3 alkylene optionally substituted with hydroxyl. In some embodiments, W is C1-C3 alkylene substituted with hydroxyl. In some embodiments, W is C1-C3 alkylene. In some embodiments, W is C3-C6 cycloalkylene or 4-12 membered heterocyclylene; each optionally substituted with 1-3 substituents independently selected from fluoro, hydroxyl, C1-C6 alkoxy, and C1-C6 alkyl. In some embodiments, W is —O—, —(NR⁴)—, —R⁵(NR⁴)—, or —(NR⁴)R⁵—. In some embodiments, W is —O— or —(NR⁴)—. In some embodiments, each R⁴ in W is hydrogen.

In some embodiments, W is —(NR⁴)(C═O)—, —R⁵(NR⁴)(C═O)—, —(C═O)(NR⁴)R⁵—, —R⁵(C═O)(NR⁴)—, or —(C═O)(NR⁴)—. In some embodiments, W is —(NR⁴)(C═O)—. In some embodiments, W is —R⁵(NR⁴)(C═O)—. In some embodiments, W is —(C═O)(NR⁴)—. In some embodiments, R⁴ within W is hydrogen. In some embodiments, each R⁴ within W is independently C1-C3 alkyl. In some embodiments, each R⁵ within W is C1-C3 alkylene. In some embodiments, W is —R⁵(C═O)—, —(C═O)R⁵—, —(C═O)—, —(S═O)—, or —S(O₂)—. In some embodiments, W is —(C═O)—. In some embodiments, W is —R⁵(C═O)— or —(C═O)R⁵—, and R⁵ is C1-C3 alkylene.

In some embodiments, X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—.

In some embodiments, X is C1-C3 alkylene. In some embodiments, X is methylene or ethylene.

In some embodiments, X is C3-C6 cycloalkylene, 4-12 membered heterocyclylene, C6-C10 arylene, or 5-10 membered heteroarylene; each optionally substituted with 1-3 substituents independently selected from fluoro, hydroxyl, C1-C6 alkoxy, and C1-C6 alkyl. In some embodiments, X is C3-C6 cycloalkylene, 4-12 membered heterocyclylene, C6-C10 arylene, or 5-10 membered heteroarylene; each substituted with 1-3 substituents independently selected from fluoro, hydroxyl, C1-C6 alkoxy, and C1-C6 alkyl. In some embodiments, X is C3-C6 cycloalkylene, 4-12 membered heterocyclylene, C6-C10 arylene, or 5-10 membered heteroarylene.

In some embodiments, X is C3-C6 cycloalkylene or 4-12 membered heterocyclylene. In some embodiments, X is 4-10 membered heterocyclylene. In some embodiments, X is 4-6 membered heterocyclylene. In some embodiments, X is selected from the group consisting of:

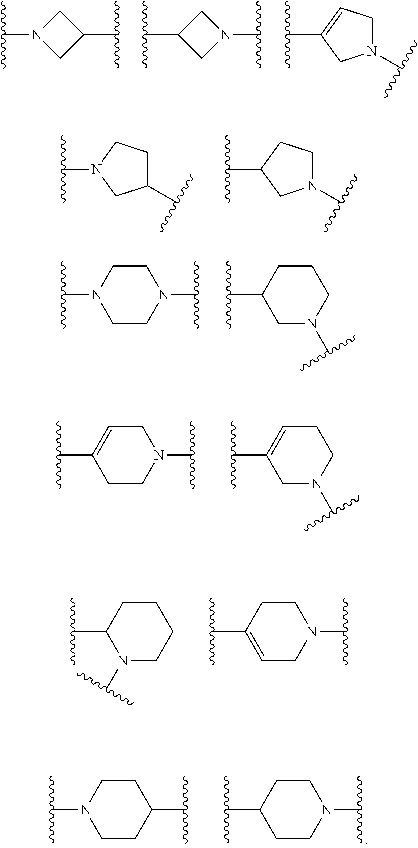

In some embodiments, X is

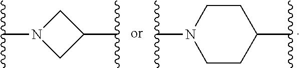

In some embodiments, X is C3-C6 cycloalkylene, such as cyclopentyl or cyclohexyl.

In some embodiments, X is 5-10 membered heteroarylene. In some embodiments, X is 5-6 membered heteroarylene. In some embodiments, V is selected from the group consisting of:

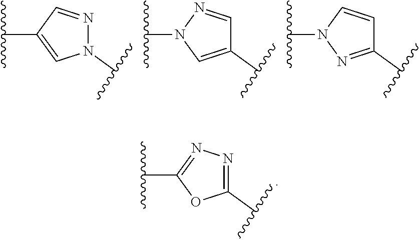 [SMILES: *c1cnn(*)c1 ; *c1cnn(*)c1 ; *c1ccn(*)n1 ; *c1nnc(*)o1]

In some embodiments, X is a C6-C10 arylene. In some embodiments, X is phenyl. In some embodiments, X is naphthyl.

In some embodiments, X is selected from the group consisting of —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, or —R⁵(NR⁴)(C═O)R⁵—. In some embodiments, X is —(C═O)R⁵— or —R⁵(C═O)—. In some embodiments, each R⁴ within X is independently hydrogen or C1-C3 alkyl. In some embodiments, each R⁴ within X is hydrogen. In some embodiments, R⁵ is C1-C3 alkylene. In some embodiments, X is a bond.

In some embodiments, U is —NR⁴(C═O)— or —(C═O)NR⁴—; V is a bond or C1-C6 alkylene; W is a bond; and X is a bond. In some embodiments, U is —NR⁴(C═O)— or —(C═O)NR⁴—; V is a bond or C1-C6 alkylene; W is a bond; and X is 4-12-membered heterocyclylene. In some embodiments, U is —NR⁴(C═O)—. In some embodiments, U is —(C═O)NR⁴—. In some embodiments, V is a bond. In some embodiments, V is C1-C3 alkylene. In some embodiments, V is methylene or ethylene.

In some embodiments, wherein U is —O—; V is C1-C6 alkylene, C3-C6 cycloalkylene, or 4-10-membered heterocyclylene; W is —C(═O)—, —N(R⁴)—, —C(═O)NR⁴—, —NR⁴C(═O)—, or —NR⁴C(═O)R⁵—. In some embodiments, V is C1-C6 alkylene. In some embodiments, V is C1-C3 alkylene. In some embodiments, V is methylene or ethylene. In some embodiments, W is —C(═O)— or —C(═O)NR⁴—. In some embodiments, W is —NR⁴C(═O)—. In some embodiments, W is —NR⁴C(═O)R⁵—. In some embodiments, each R⁴ within W is hydrogen. In some embodiments, each R⁵ within W is independently C1-C3 alkylene.

In some embodiments, U is —NR⁴—; V is C1-C6 alkylene or a bond; W is —C(═O)— or —C(═O)R⁵—; and X is a bond. In some embodiments, U is —NH—. In some embodiments, U is —N(C1-C3 alkyl)-. In some embodiments, V is C1-C3 alkylene. In some embodiments, V is methylene or ethylene. In some embodiments, W is —C(═O)—. In some embodiments, W is —C(═O)R⁵—. In some embodiments, each R⁵ within W is independently C1-C3 alkylene.

In some embodiments, U is a bond, C1-C3 alkylene, C2-C3 alkenylene, or C2-C3 alkynylene; V is a bond; W is a bond or C(═O); and X is a bond or C6-C10 arylene. In some embodiments, U is a bond. In some embodiments, U is C2-C3 alkenylene. In some embodiments, U is C2-C3 alkynylene. In some embodiments, W is a bond. In some embodiments, W is C(═O). In some embodiments, X is a bond. In some embodiments, X is C6-C10 arylene.

In some embodiments, Y is R⁶, R⁶(CR^{A}R^{B})ₚ-Q-, or -Q-(CR^{A}R^{B})ₚR⁶—.

In some embodiments, Y is R⁶. In some embodiments, R⁶ is 4-12 membered heterocyclylene optionally substituted with 1-3 substituents independently selected from fluoro, hydroxyl, C1-C6 alkoxy, and C1-C6 alkyl. In some embodiments, R⁶ is 4-8 membered heterocyclylene optionally substituted with 1-3 substituents independently selected from fluoro, hydroxyl, C1-C6 alkoxy, and C1-C6 alkyl. In some embodiments, R⁶ is 4-6 membered heterocyclylene optionally substituted with 1-3 substituents independently selected from fluoro, hydroxyl, C1-C6 alkoxy, and C1-C6 alkyl.

In some embodiments, R⁶ is 4-12 membered heterocyclylene substituted with 1-3 substituents independently selected from fluoro, hydroxyl, C1-C6 alkoxy, and C1-C6 alkyl. In some embodiments, R⁶ is 4-8 membered heterocyclylene substituted with 1-3 substituents independently selected from fluoro, hydroxyl, C1-C6 alkoxy, and C1-C6 alkyl. In some embodiments, R⁶ is 4-6 membered heterocyclylene substituted with 1-3 substituents independently selected from fluoro, hydroxyl, C1-C6 alkoxy, and C1-C6 alkyl.

In some embodiments, R⁶ is 4-8 membered heterocyclylene substituted with hydroxyl. In some embodiments, R⁶ is 4-8 membered heterocyclylene substituted with C1-C6 alkyl, such as methyl. In some embodiments, R⁶ is 4-8 membered heterocyclylene substituted with fluoro. In some embodiments, R⁶ is 4-8 membered heterocyclylene substituted with two fluoros.

In some embodiments, R⁶ is 4-12 membered heterocyclylene. In some embodiments, R⁶ is 4-8 membered heterocyclylene. In some embodiments, R⁶ is 4-6 membered heterocyclylene.

In some embodiments, R⁶ is selected from the group consisting of:

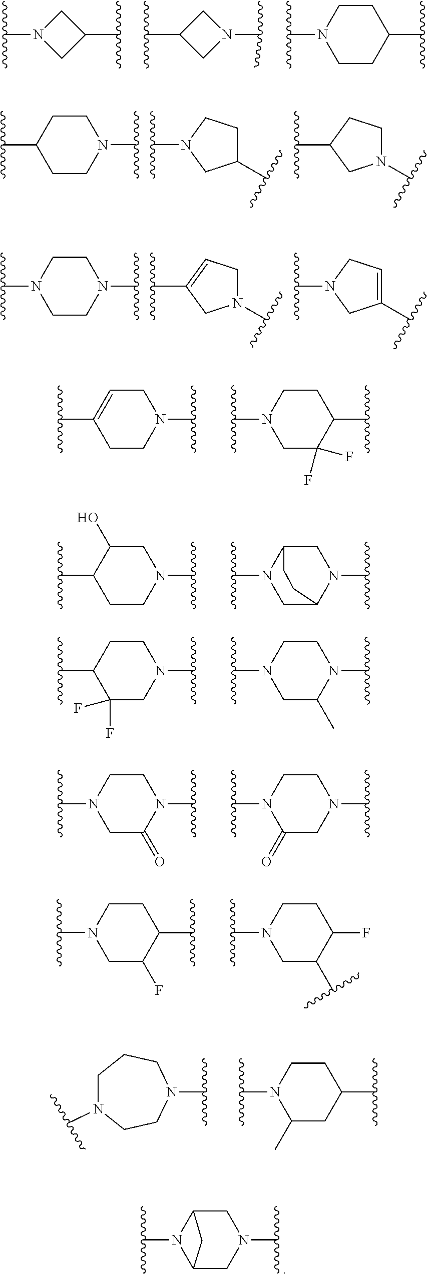

In some embodiments, R⁶ is

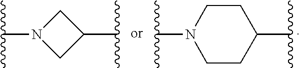

In some embodiments, R⁶ is

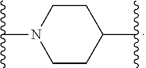 [SMILES: *C1CCN(*)CC1 ; "."]

In some embodiments, R⁶ is 7-12 membered bicyclic heterocyclylene. In some embodiments, R⁶ is 7-12 membered bicyclic spirocyclic heterocyclylene. In some embodiments, R⁶ is

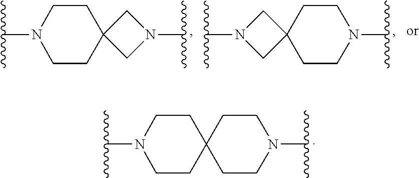

In some embodiments, R⁶ is 5-10 membered heteroarylene optionally substituted with 1-3 substituents independently selected from fluoro, hydroxyl, C1-C6 alkoxy, and C1-C6 alkyl. In some embodiments, R⁶ is 5-6 membered heteroarylene optionally substituted with 1-3 substituents independently selected from fluoro, hydroxyl, C1-C6 alkoxy, and C1-C6 alkyl. In some embodiments R⁶ is 5-6 membered heteroarylene. In some embodiments, R⁶ is selected from the group consisting of:

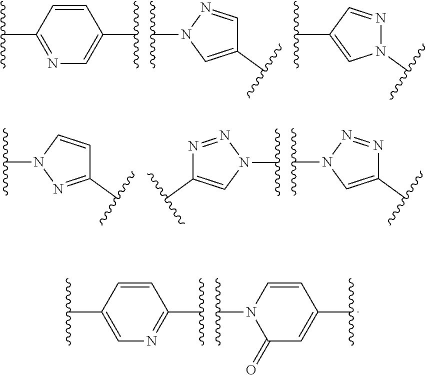

In some embodiments, R⁶ is C1-C3 alkylene.

In some embodiments, —Y— is —R⁶(CR^{A}R^{B})ₚ-Q-. In some embodiments, —Y— is -Q-(CR^{A}R^{B})ₚR⁶—. In some embodiments, -Q- is —(NR⁴)—. In some embodiments, R⁴ is hydrogen. In some embodiments, R⁴ is C1-C3 alkyl. In some embodiments, -Q- is —O—.

In some embodiments, p is 0, 1, or 2. In some embodiments, p is 0 or 1. In some embodiments, p is 1 or 2. p is 0. In some embodiments, p is 1. In some embodiments, p is 2.

In some embodiments, each R^{A} and R^{B} are independently hydrogen, fluoro, or C1-C3 alkyl. In some embodiments, one pair of R^{A} and R^{B}, on the same carbon, combine to form oxo. In some embodiments, each R^{A} and R^{B} are hydrogen. In some embodiments, 1 or 2 of R^{A} and R^{B} are independently fluoro or C1-C3 alkyl; and each remaining R^{A} and R^{B} is hydrogen. In some embodiments, one pair of R^{A} and R^{B}, on the same carbon, combine to form oxo; and each remaining R^{A} and R^{B}, if present, are hydrogen.

In some embodiments, Y is —R⁶(CR^{A}R^{B})ₚ-Q-; and p is 0. In some embodiments, Y is —R⁶NR⁴— or —R⁶O—. In some embodiments, Y is —R⁶NR⁴—. In some embodiments, Y is —R⁶O—. In some embodiments, Y is R⁶(CR^{A}R^{B})ₚ-Q- or -Q-(CR^{A}R^{B})ₚR⁶; p is 1 or 2; and each R^{A} and R^{B} are hydrogen. In some embodiments, Y is —R⁶CH₂—O— or —R⁶CH₂—N(R⁴)—. In some embodiments, Y is —R⁶CH₂—O—. In some embodiments, Y is —R⁶CH₂—NH.

In some embodiments, Y is —R⁶(CR^{A}R^{B})ₚ-Q- or -Q-(CR^{A}R^{B})ₚR⁶—; p is 1 or 2; and each R^{A} and R^{B} are independently hydrogen or C1-C3 alkyl; or one pair of R^{A} and R^{B}, together with the carbon atom to which they are attached, come together to form a C3-C4 cycloalkyl, and each remaining R^{A} and R^{B}, if present, are hydrogen. In some embodiments, Y is —R⁶(CR^{A}R^{B})ₚ-Q-. In some embodiments, Y is -Q-(CR^{A}R^{B})ₚR⁶—.

In some embodiments, the —(CR^{A}R^{B})ₚ-Q- portion of Y is selected from the group consisting of:

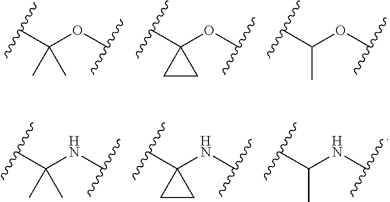

In some embodiments, Y is —R⁶C(═O)(CR^{A}R^{B})-Q-; and each R^{A} and R^{B} are independently hydrogen, fluoro, or C1-C3 alkyl. In some embodiments, Y is -Q-(CR^{A}R^{B})ₚR⁶—; and each R^{A} and R^{B} are independently hydrogen, fluoro, or C1-C3 alkyl. In some embodiments, the —(CR^{A}R^{B})ₚ-Q- portion of Y is selected from the group consisting of:

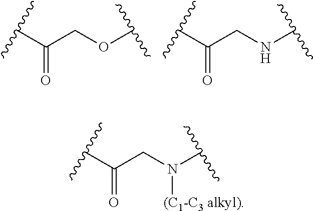

In some embodiments, R⁶ is 5-10 membered heteroarylene optionally substituted with 1-3 substituents independently selected from fluoro, hydroxyl, C1-C6 alkoxy, and C1-C6 alkyl. In some embodiments, R⁶ is 5-6 membered heteroarylene optionally substituted with 1-3 substituents independently selected from fluoro, hydroxyl, C1-C6 alkoxy, and C1-C6 alkyl. In some embodiments, R⁶ is 5-10 membered heteroarylene substituted with 1-3 substituents independently selected from fluoro, hydroxyl, C1-C6 alkoxy, and C1-C6 alkyl. In some embodiments, R⁶ is 5-6 membered heteroarylene substituted with 1-3 substituents independently selected from fluoro, hydroxyl, C1-C6 alkoxy, and C1-C6 alkyl. In some embodiments, R⁶ is 5-10 membered heteroarylene. In some embodiments, R⁶ is 5-6 membered heteroarylene.

In some embodiments, R⁶ is 5-6 membered heteroarylene. In some embodiments, R⁶ is triazolylene, pyrazolylene, or pyridinylene. In some embodiments, R⁶ is selected from the group consisting of:

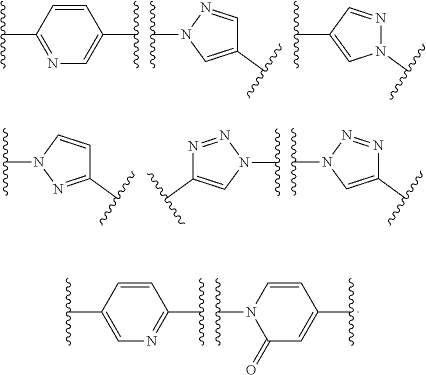

In some embodiments, R⁶ is C6-C10 arylene. In some embodiments, R⁶ is phenylene.

In some embodiments, Z is:

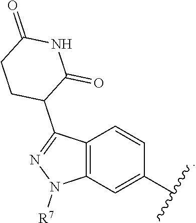

In some embodiments, Z is selected from the group consisting of:

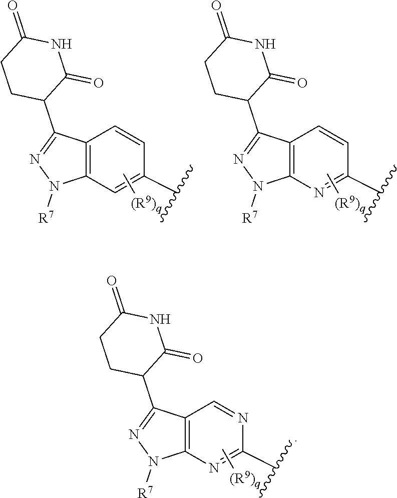

In some embodiments, Z is:

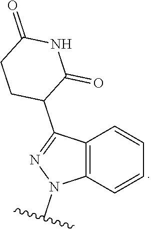 [SMILES: *n1nc(C2CCC(=O)NC2=O)c2ccccc21]

In some embodiments, Z is

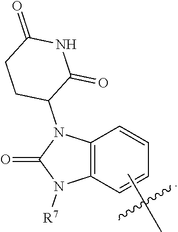

In some embodiments, Z is:

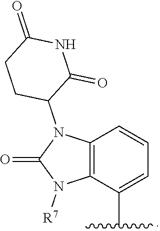

In some embodiments, Z is:

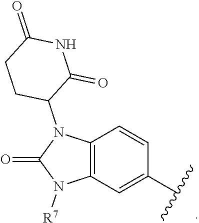

In some embodiments, Z is selected from the group consisting of:

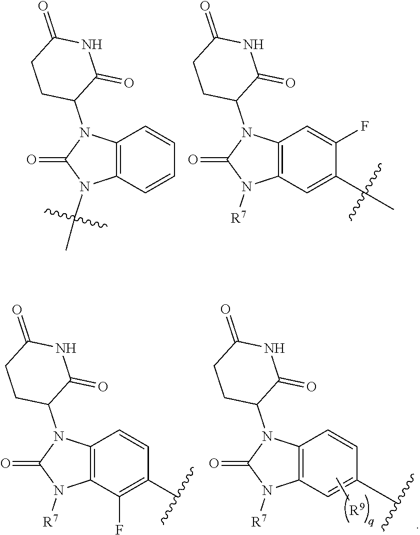

In some embodiments, Z is:

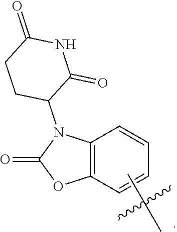

In some embodiments, Z is:

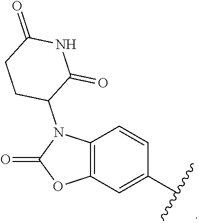 [SMILES: *c1ccc2c(c1)oc(=O)n2C1CCC(=O)NC1=O]

In some embodiments, Z is:

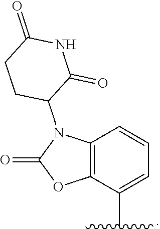 [SMILES: *c1cccc2c1oc(=O)n2C1CCC(=O)NC1=O]

In some embodiments, Z is selected from the group consisting of:

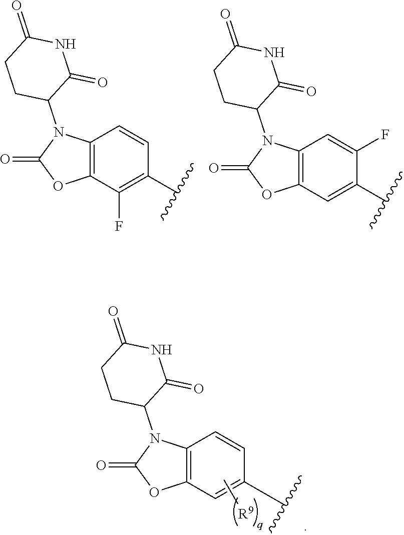

In some embodiments, Z is selected from the group consisting of:

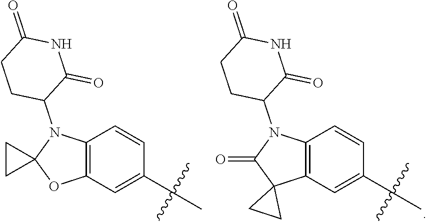 [SMILES: *c1ccc2c(c1)OC1(CC1)N2C1CCC(=O)NC1=O ; *c1ccc2c(c1)C1(CC1)C(=O)N2C1CCC(=O)NC1=O]

In some embodiments, Z is selected from the group consisting of:

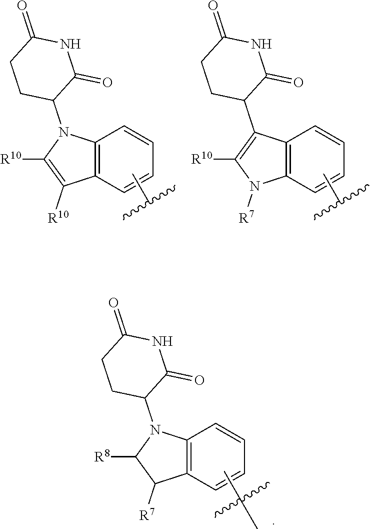

In some embodiments, Z is

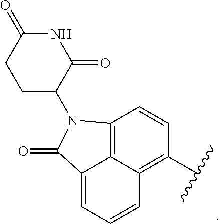 [SMILES: *c1ccc2c3c(cccc13)C(=O)N2C1CCC(=O)NC1=O]

In some embodiments, Z is

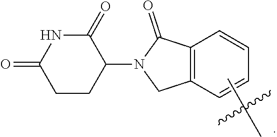

In some embodiments, Z is

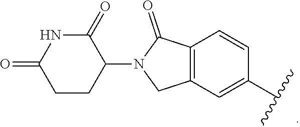 [SMILES: *c1ccc2c(c1)CN(C1CCC(=O)NC1=O)C2=O]

In some embodiments, Z is

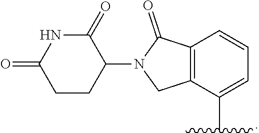 [SMILES: *c1cccc2c1CN(C1CCC(=O)NC1=O)C2=O]

In some embodiments, Z is

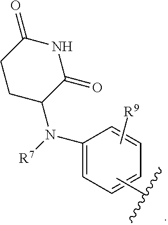

In some embodiments, Z is

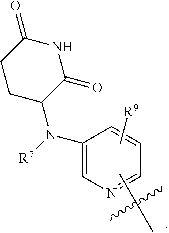

In some embodiments, R⁷, if present, is hydrogen. In some embodiments, R⁷, if present, is C1-C6 alkyl. In some embodiments, R⁷, if present, is C1-C3 alkyl. In some embodiments, R⁷, if present, is methyl. In some embodiments, R⁷, if present, is C1-C6 alkyl substituted with one group selected from hydroxyl, cyano and C1-C6 alkoxy. In some embodiments, R⁷, if present, is C1-C6 haloalkyl. In some embodiments, R⁷, if present, is C3-C6 cycloalkyl, or 4-6 membered heterocyclyl, —(CR^{A}R^{B})(4-12 membered heterocyclyl), or —(CR^{A}R^{B})(C3-C6 cycloalkyl). The In some embodiments, each R^{A} and R^{B} are hydrogen.

In some embodiments, R⁸, if present, is hydrogen. In some embodiments, R⁸, if present, is C1-C6 alkyl. In some embodiments, R⁸, if present, is C1-C3 alkyl.

In some embodiments, q is 0 or 1. In some embodiments, q is 0. In some embodiments, q is 1.

In some embodiments, R⁹, if present, is hydrogen. In some embodiments, R⁹, if present, is halogen. In some embodiments, R⁹, if present, is cyano. In some embodiments, R⁹, if present, is C1-C6 alkyl or C1-C6 haloalkyl. In some embodiments, R⁹, if present, is C1-C6 alkoxy, C1-C5 cycloalkoxy, 5-10 membered heteroaryloxy, or phenoxy.

In some embodiments, each R¹⁰, when present, is hydrogen. In some embodiments, one R¹⁰ is cyano, and the remaining R¹⁰, if present, are hydrogen. In some embodiments, one R¹⁰ is halogen, and the remaining R¹⁰, if present, are hydrogen. In some embodiments, the halogen is fluoro. In some embodiments, one R¹⁰ is C1-C6 alkyl, C1-C6 haloalkyl, or C3-C6 cycloalkyl, and the remaining R¹⁰, if present, are hydrogen.

In some embodiments, the compound of Formula (I) is a compound of Formula (I-a):

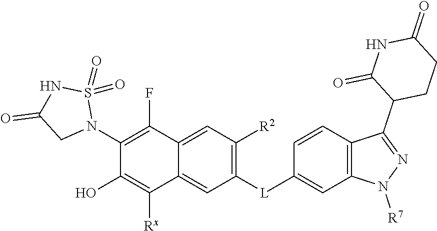

or a pharmaceutically acceptable salt thereof.

In some embodiments, the compound of Formula (I) is a compound of Formula (I-b):

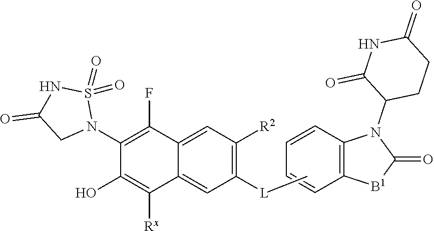

or a pharmaceutically acceptable salt thereof; wherein B¹ is O or NR⁷.
In some embodiments, the compound of Formula (I-b) is a compound of Formula (I-b1):

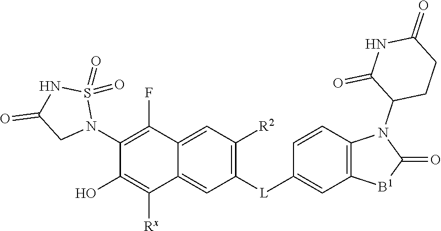

or a pharmaceutically acceptable salt thereof.
In some embodiments, the compound of Formula (I-b) is a compound of Formula (I-b2):

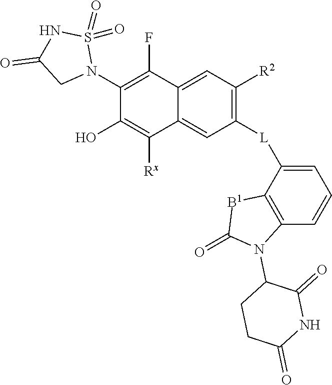

or a pharmaceutically acceptable salt thereof.

In some embodiments of a compound of Formula (I-b), B¹ is NR⁷. In some embodiments of a compound of Formula (I-b), R⁷ is C1-C3 alkyl. In some embodiments of a compound of Formula (I-b), R⁷ is methyl, ethyl, or isopropyl. In some embodiments of a compound of Formula (I-b), R⁷ is methyl. In some embodiments of a compound of Formula (I-b), R⁷ is hydrogen. In some embodiments, B¹ is O.

In some embodiments, the compound of Formula (I) is a compound of Formula (I-c):

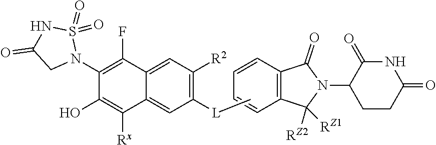

or a pharmaceutically acceptable salt thereof; wherein R^{Z1} and R^{Z2} are both hydrogen; or R^{Z1} and R^{Z2} combine to form oxo.

In some embodiments, the compound of Formula (I-c) is a compound of Formula (I-c1):

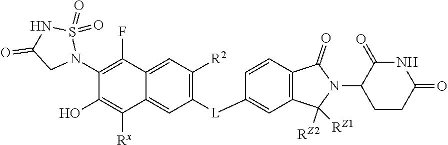

or a pharmaceutically acceptable salt thereof.

In some embodiments, the compound of Formula (I-c) is a compound of Formula (I-c2):

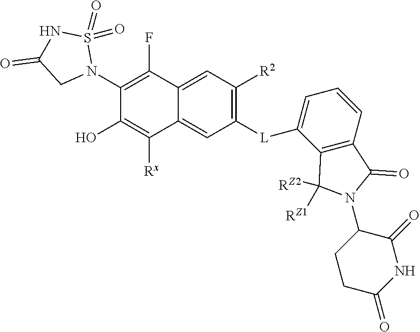

or a pharmaceutically acceptable salt thereof.

In some embodiments of a compound of Formula (I-c2), both R^{Z1} and R²² are hydrogen. In some embodiments of a compound of Formula (I-c2), R^{Z1} and R^{Z2} combine to form oxo.

In some embodiments, the compound of Formula (I) is a compound of Formula (I-d):

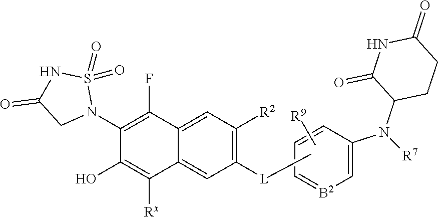

or a pharmaceutically acceptable salt thereof, wherein B² is CH or N. In some embodiments of a compound of Formula (I-d), B² is CH. In some embodiments of a compound of Formula (I-d), R⁹ is hydrogen. In some embodiments of a compound of Formula (I-d), R⁹ is halogen. In some embodiments of a compound of Formula (I-d), R⁹ is fluoro. In some embodiments of a compound of Formula (I-d), R⁷ is hydrogen.

In some embodiments, the compound of Formula (I) is a compound of Formula (I-e):

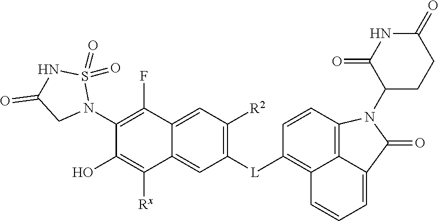

or a pharmaceutically acceptable salt thereof. In some embodiments of a compound of Formula (I-e), R² is hydrogen. In some embodiments of a compound of Formula (I-e), R² is halogen. In some embodiments of a compound of Formula (I-e), R² is C1-C3 alkoxy, C3-C6 cycloalkoxy, C1-C3 haloalkoxy, C1-C3 haloalkyl, or C3-C5 halocycloalkoxy. In some embodiments of a compound of Formula (I-e), R² is C1-C3 alkyl or C3-C6 cycloalkyl.

In some embodiments, the compound of Formula (I) is a compound of Formula (II-a):

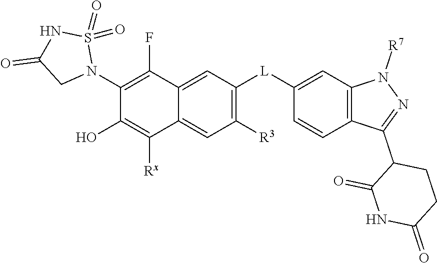

or a pharmaceutically acceptable salt thereof.

In some embodiments, the compound of Formula (I) is a compound of Formula (II-b):

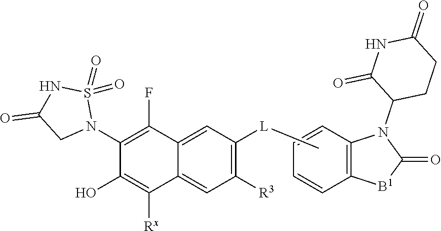

or a pharmaceutically acceptable salt thereof; wherein B¹ is O or NR⁷.

In some embodiments, the compound of Formula (II-b) is a compound of Formula (II-b1):

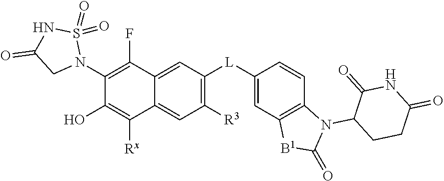

or a pharmaceutically acceptable salt thereof.

In some embodiments, the compound of Formula (II-b) is a compound of Formula (II-b2):

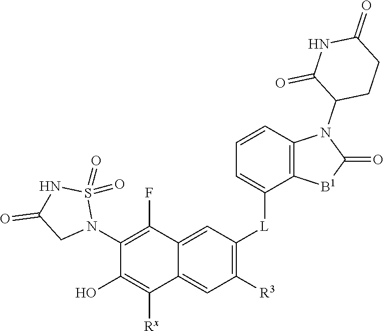

or a pharmaceutically acceptable salt thereof. In some embodiments of a compound of Formula (II-b2), B¹ is NR⁷. In some embodiments of a compound of Formula (II-b2), R⁷ is C1-C3 alkyl. In some embodiments of a compound of Formula (II-b2), R⁷ is methyl, ethyl, or isopropyl. In some embodiments of a compound of Formula (II-b2), R⁷ is methyl. In some embodiments of a compound of Formula (II-b2), R⁷ is hydrogen. In some embodiments of a compound of Formula (II-b2), B¹ is O.

In some embodiments, the compound of Formula (I) is a compound of Formula (II-c):

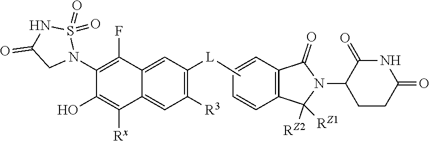

or a pharmaceutically acceptable salt thereof; wherein R^{Z1} and R²² are both hydrogen; or R^{Z1} and R^{Z2} combine to form oxo.

In some embodiments, the compound of Formula (II-c) is a compound of Formula (II-c1):

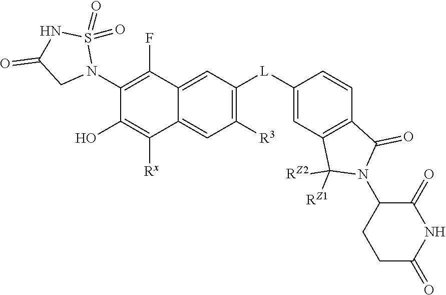

or a pharmaceutically acceptable salt thereof.

In some embodiments, the compound of Formula (II-c) is a compound of Formula (II-c2):

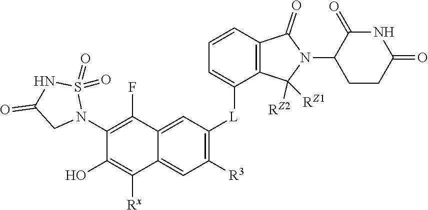

or a pharmaceutically acceptable salt thereof. In some embodiments, both R^{Z1} and R^{Z2} are hydrogen. In some embodiments of a compound of Formula (II-c2), R^{Z1} and R^{Z2} combine to form oxo.

In some embodiments, the compound of Formula (I) is a compound of Formula (II-d):

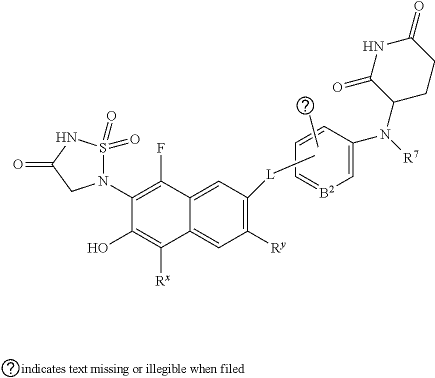

or a pharmaceutically acceptable salt thereof, wherein B² is CH or N. In some embodiments of a compound of Formula (II-d), B² is CH. In some embodiments of a compound of Formula (II-d), R′ is hydrogen. In some embodiments of a compound of Formula (II-d), R′ is halogen. In some embodiments of a compound of Formula (II-d), R⁹ is fluoro. In some embodiments of a compound of Formula (II-d), R⁷ is hydrogen.

In some embodiments, the compound of Formula (I) is a compound of Formula (II-e):

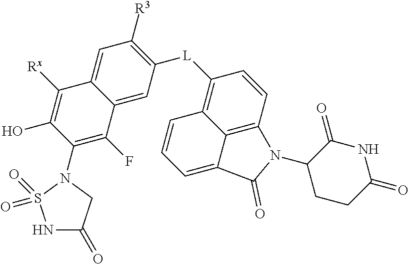

or a pharmaceutically acceptable salt thereof. In some embodiments of a compound of Formula (II-e), R³ is hydrogen. In some embodiments of a compound of Formula (II-e), R³ is halogen. In some embodiments of a compound of Formula (II-e), R³ is C1-C3 alkoxy, C3-C6 cycloalkoxy, C1-C3 haloalkoxy, or C3-C5 halocycloalkoxy. In some embodiments of a compound of Formula (II-e), R³ is C1-C3 alkyl or C3-C6 cycloalkyl. In some embodiments of a compound of Formula (II-e), R^{x} is hydrogen. In some embodiments of a compound of Formula (II-e), R^{x} is halogen.

In some embodiments of compounds of Formula (I-a) to Formula (II-e), L is —U—V—W—X—Y—. In some embodiments of compounds of Formula (I-a) to Formula (II-e), U is —NR⁴(C═O)— or —(C═O)NR⁴—; V is a bond or C1-C6 alkylene; W is a bond; and X is a bond. In some embodiments of compounds of Formula (I-a) to Formula (II-e), U is —NR⁴(C═O)— or —(C═O)NR⁴—; V is a bond or C1-C6 alkylene; W is a bond; and X is 4-12-membered heterocyclylene. In some embodiments of compounds of Formula (I-a) to Formula (II-e), U is —NR⁴(C═O)—. In some embodiments of compounds of Formula (I-a) to Formula (II-e), U is —(C═O)NR⁴—. In some embodiments of compounds of Formula (I-a) to Formula (II-e), V is a bond. In some embodiments of compounds of Formula (I-a) to Formula (II-e), V is C1-C3 alkylene. In some embodiments of compounds of Formula (I-a) to Formula (II-e), V is methylene or ethylene.

In some embodiments of compounds of Formula (I-a) to Formula (II-e), U is —O—; V is C1-C6 alkylene, C3-C6 cycloalkylene, or 4-10-membered heterocyclylene; W is —C(═O)—, —N(R⁴)—, —C(═O)NR⁴—, —NR⁴C(═O)—, —NR⁴C(═O)R⁵—, or —(S═O)—, or —S(O₂)—. In some embodiments of compounds of Formula (I-a) to Formula (II-e), V is C1-C6 alkylene. In some embodiments of compounds of Formula (I-a) to Formula (II-e), V is C1-C3 alkylene. In some embodiments of compounds of Formula (I-a) to Formula (II-e), V is methylene or ethylene. In some embodiments of compounds of Formula (I-a) to Formula (II-e), W is —C(═O)— or —C(═O)NR⁴—. In some embodiments of compounds of Formula (I-a) to Formula (II-e), W is —NR⁴C(═O)—. In some embodiments of compounds of Formula (I-a) to Formula (II-e), W is —NR⁴C(═O)R⁵—. In some embodiments of compounds of Formula (I-a) to Formula (II-e), R⁴ is hydrogen. In some embodiments of compounds of Formula (I-a) to Formula (II-e), R⁵ is C1-C3 alkylene.

In some embodiments of compounds of Formula (I-a) to Formula (II-e), U is —NR⁴—; V is C1-C6 alkylene or a bond; W is —C(═O)— or —C(═O)R⁵—; and X is a bond. In some embodiments of compounds of Formula (I-a) to Formula (II-e), U is —NH—. In some embodiments of compounds of Formula (I-a) to Formula (II-e), U is —N(C1-C3 alkyl)-. In some embodiments of compounds of Formula (I-a) to Formula (II-e), V is C1-C3 alkylene. In some embodiments of compounds of Formula (I-a) to Formula (II-e), V is methylene or ethylene. In some embodiments of compounds of Formula (I-a) to Formula (II-e), W is —C(═O)—. In some embodiments of compounds of Formula (I-a) to Formula (II-e), W is —C(═O)R⁵—. In some embodiments of compounds of Formula (I-a) to Formula (II-e), R⁵ is C1-C3 alkylene.

In some embodiments of compounds of Formula (I-a) to Formula (II-e), U is a bond, C1-C3 alkylene, C2-C3 alkenylene, or C2-C3 alkynylene; V is a bond; W is a bond or C(═O); and X is a bond or C6-C10 arylene. In some embodiments of compounds of Formula (I-a) to Formula (II-e), U is a bond. In some embodiments of compounds of Formula (I-a) to Formula (II-e), U is C2-C3 alkenylene. In some embodiments of compounds of Formula (I-a) to Formula (II-e), U is C2-C3 alkynylene. In some embodiments of compounds of Formula (I-a) to Formula (II-e), W is a bond. In some embodiments of compounds of Formula (I-a) to Formula (II-e), W is C(═O). In some embodiments of compounds of Formula (I-a) to Formula (II-e), X is a bond. In some embodiments of compounds of Formula (I-a) to Formula (II-e), X is C6-C10 arylene.

In some embodiments of compounds of Formula (I-a) to Formula (II-e), Y is R⁶. In some embodiments of compounds of Formula (I-a) to Formula (II-e), R⁶ is 4-12 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-e), R⁶ is 4-8 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-e), R⁶ is selected from the group consisting of:

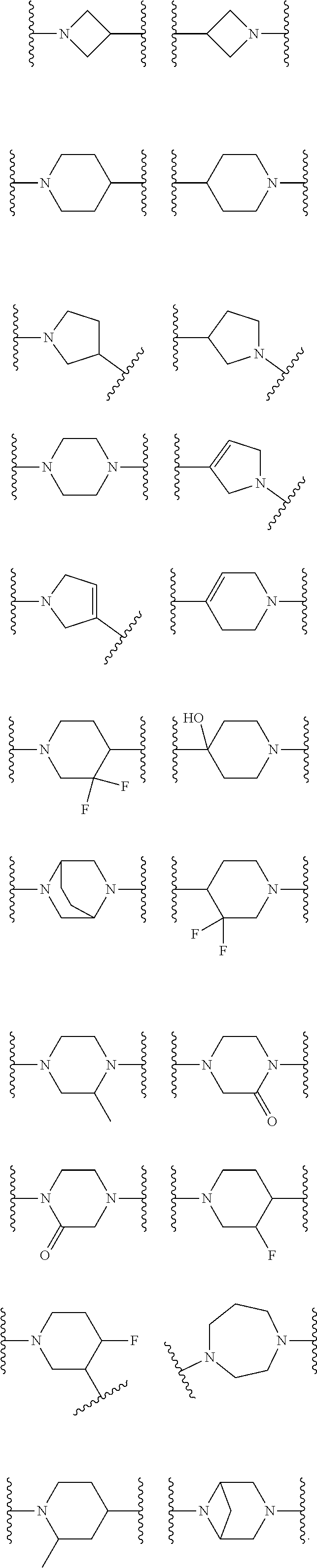

In some embodiments of compounds of Formula (I-a) to Formula (II-e), R⁶ is

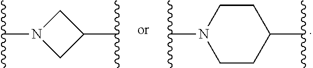 [SMILES: *C1CN(*)C1 ; *C1CCN(*)CC1]

In some embodiments of compounds of Formula (I-a) to Formula (II-e), R⁶ is

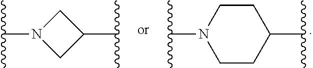 [SMILES: *C1CN(*)C1 ; *C1CCN(*)CC1]

In some embodiments of compounds of Formula (I-a) to Formula (II-e), R⁶ is 7-12 membered bicyclic heterocyclylene. In some embodiments of compounds of Formula (I-a) to Formula (II-e), R⁶ is 7-12 membered bicyclic spirocyclic heterocyclylene. In some embodiments of compounds of Formula (I-a) to Formula (II-e), R⁶ is

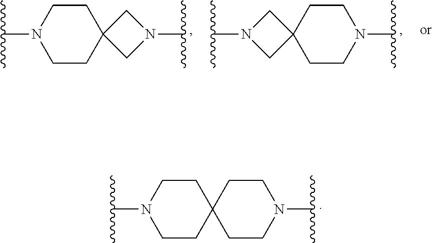

In some embodiments of compounds of Formula (I-a) to Formula (II-e), R⁶ is C1-C3 alkylene.

In some embodiments of Formula (I-a) to Formula (II-e), R⁶ is 5-10 membered heteroarylene. In some embodiments of Formula (I-a) to Formula (II-e), R⁶ is 5-6 membered heteroarylene. In some embodiments of Formula (I-a) to Formula (II-e), R⁶ is selected from the group consisting of:

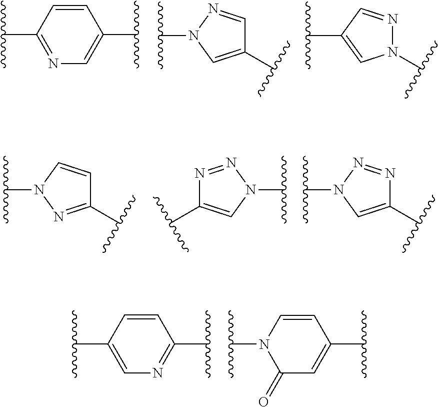

In some embodiments of compounds of Formula (I-a) to Formula (II-e), R⁶ is C1-C3 alkylene.

In some embodiments of compounds of Formula (I-a) to Formula (II-e), Y is —R⁶(CR^{A}R^{B})ₚ-Q-; and p is 0. In some embodiments of compounds of Formula (I-a) to Formula (II-e), Y is —R⁶NR⁴— or —R⁶O—. In some embodiments of compounds of Formula (I-a) to Formula (II-e), Y is —R⁶NH. In some embodiments of compounds of Formula (I-a) to Formula (II-e), Y is —R⁶O—. In some embodiments of compounds of Formula (I-a) to Formula (II-e), Y is R⁶(CR^{A}R^{B})ₚ-Q- or -Q-(CR^{A}R^{B})ₚR⁶—; p is 1 or 2; and each R^{A} and R^{B} are hydrogen. In some embodiments of compounds of Formula (I-a) to Formula (II-e), Y is —R⁶CH₂—O— or —R⁶CH₂—N(R⁴)—. In some embodiments of compounds of Formula (I-a) to Formula (II-e), Y is —R⁶CH₂—O—. In some embodiments of compounds of Formula (I-a) to Formula (II-e), Y is —R⁶CH₂—NH. In some embodiments of compounds of Formula (I-a) to Formula (II-e), Y is —R⁶(CR^{A}R^{B})ₚ-Q- or -Q-(CR^{A}R^{B})ₚR⁶—; p is 1 or 2; and each R^{A} and R^{B} are independently hydrogen or C1-C3 alkyl; or one pair of R^{A} and R^{B}, together with the carbon atom to which they are attached, come together to form a C3-C4 cycloalkyl, and each remaining R^{A} and R^{B}, if present, are hydrogen.

In some embodiments of compounds of Formula (I-a) to Formula (II-e), the —(CR^{A}R^{B})ₚ-Q- portion of Y is selected from the group consisting of:

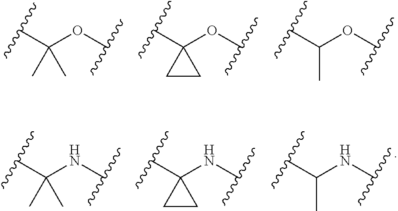

In some embodiments of compounds of Formula (I-a) to Formula (II-e), Y is —R⁶C(═O)(CR^{A}R^{B})-Q-; and each R^{A} and R^{B} are independently hydrogen, fluoro, or C—C3 alkyl. In some embodiments of compounds of Formula (I-a) to Formula (II-e), the —(CR^{A}R^{B})ₚ-Q- portion of Y is selected from the group consisting of:

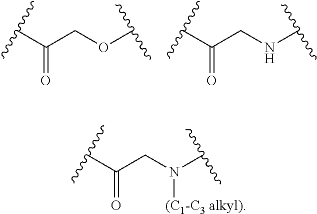

In some embodiments of compounds of Formula (I-a) to Formula (II-e), R⁶ is 4-12 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-e), R⁶ is 4-8 membered heterocyclylene optionally substituted with 1-3 substituents independently selected from fluoro, hydroxyl, C1-C6 alkoxy, and C1-C6 alkyl. In some embodiments of compounds of Formula (I-a) to Formula (II-e), R⁶ is selected from the 5 group consisting of:

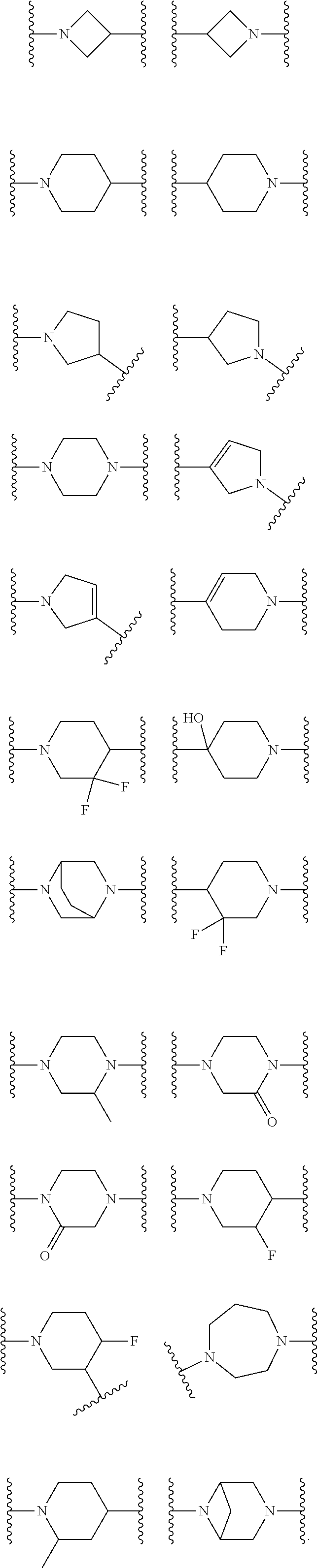

In some embodiments of compounds of Formula (I-a) to Formula (II-e), R⁶ is

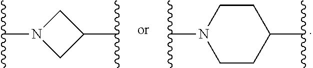 [SMILES: *C1CN(*)C1 ; *C1CCN(*)CC1]

In some embodiments of compounds of Formula (I-a) to Formula (II-e), R⁶ is

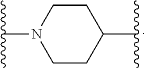 [SMILES: *C1CCN(*)CC1]

In some embodiments of compounds of Formula (I-a) to Formula (II-e), R⁶ is 7-12 membered bicyclic heterocyclylene. In some embodiments of compounds of Formula (I-a) to Formula (II-e), R⁶ is 7-12 membered bicyclic spirocyclic heterocyclylene. In some embodiments of compounds of Formula (I-a) to Formula (II-e), R⁶ is

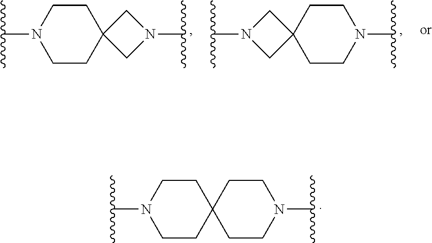

In some embodiments of compounds of Formula (I-a) to Formula (II-e), R⁶ is C6-C10 arylene. In some embodiments of compounds of Formula (I-a) to Formula (II-e), R⁶ is phenylene.

In some embodiments of compounds of Formula (I-a) to Formula (II-e), R⁶ is 5-10 membered heteroarylene. In some embodiments of compounds of Formula (I-a) to Formula (II-e), R⁶ is 5-6 membered heteroarylene. In some embodiments of compounds of Formula (I-a) to Formula (II-e), R⁶ is 5-6 membered heteroarylene. In some embodiments of compounds of Formula (I-a) to Formula (II-e), R⁶ is triazolylene, pyrazolylene, or pyridinylene. In some embodiments of compounds of Formula (I-a) to Formula (II-e), R⁶ is selected from the group consisting of:

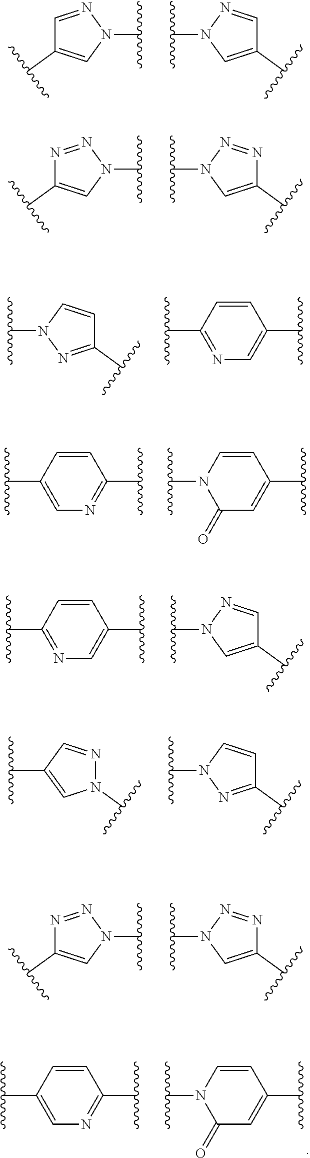

In some embodiments, R¹ is fluoro; R^{x} is hydrogen; R² is hydrogen; R³ is -L-Z; Z is

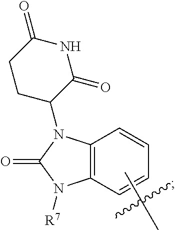

and R⁷ is hydrogen or C1-C6 alkyl.

In some embodiments, R¹ is fluoro; R is hydrogen; R² is -L-Z; R³ is hydrogen;

### Z is

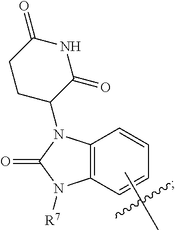

and R⁷ is hydrogen or C1-C6 alkyl.

In some embodiments:
U is —(NR⁴)C═O)—, —(C═O)NR⁴—, or —(NR⁴)(C═O)(NR⁴)—;
V is a bond, C1-C6 alkylene, or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is C3-C7 cycloalkylene, 4-12 membered heterocyclylene, C6-C10 arylene, or 5-10 membered heteroarylene; and
R⁴ is hydrogen or C1-C6 alkyl.

In some embodiments:
U is —(NR⁴)C═O)—, —(C═O)NR⁴—, or —(NR⁴)(C═O)(NR⁴)—;
V is a bond or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is 4-8 membered heterocyclylene, phenyl, or 5-6 membered heteroarylene; and
R⁴ is hydrogen or C1-C6 alkyl.

In some embodiments, V and X are bonds.

In some embodiments, R⁶ is piperidinyl, piperazinyl, phenyl, pyridinyl, or pyridonyl.

In some embodiments, W is C1-C3 alkylene and R⁴ is hydrogen.

In some embodiments, U is —(NR⁴)C═O)—, V is a bond, W is C1-C3 alkylene, X is a bond, and Y is R⁶.

In some embodiments, R⁴ is hydrogen or methyl; and R⁶ is 5-6 membered heterocyclylene, phenyl, or 5-6 membered heteroarylene.

In some embodiments, R⁶ is piperidinyl, piperazinyl, phenyl, pyridinyl, or pyridonyl.

In some embodiments, R¹ is fluoro; R^{x} is hydrogen; R² is hydrogen; R³ is -L-Z; Z is

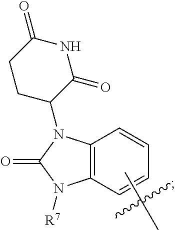

R⁷ is hydrogen or C1-C6 alkyl; L is —U—V—W—X—Y—; U is —(NH)C═O)—, —(C═O)NH—, or —(NH)(C═O)(NH)—; V is a bond; W is methylene or ethylene; X is a bond; Y is R⁶; and R⁶ is piperidinyl, piperazinyl, phenyl, pyridinyl, or pyridonyl.

In some embodiments, R¹ is fluoro; R^{x} is hydrogen; R² is -L-Z; R³ is hydrogen; Z is

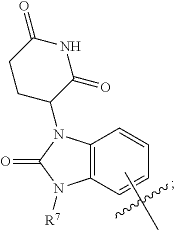

R⁷ is hydrogen or C1-C6 alkyl; L is —U—V—W—X—Y—; U is —(NH)C═O)—, —(C═O)NH—, or —(NH)(C═O)(NH)—; V is a bond; W is methylene or ethylene; X is a bond; Y is R⁶; and R⁶ is piperidinyl, piperazinyl, phenyl, pyridinyl, or pyridonyl.

In some embodiments, L is selected from the group consisting of:

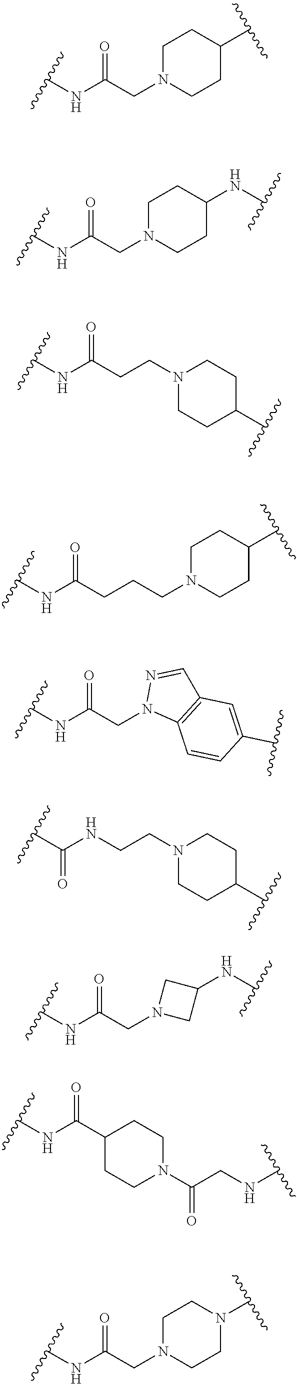

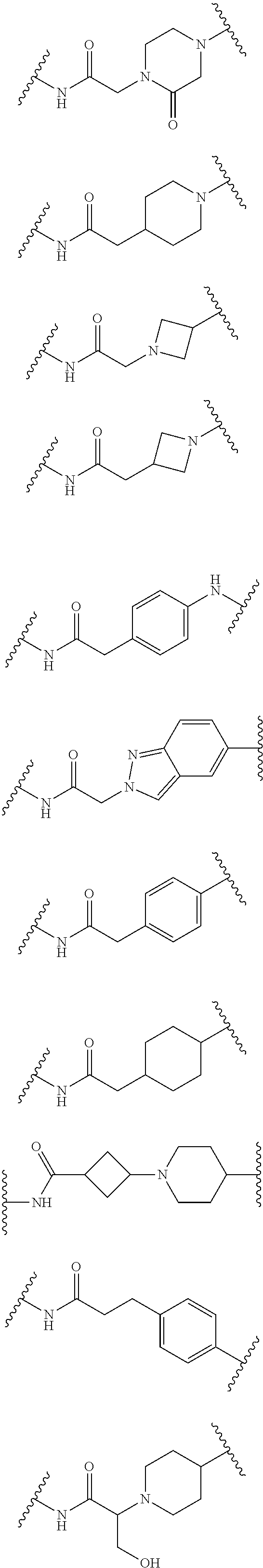

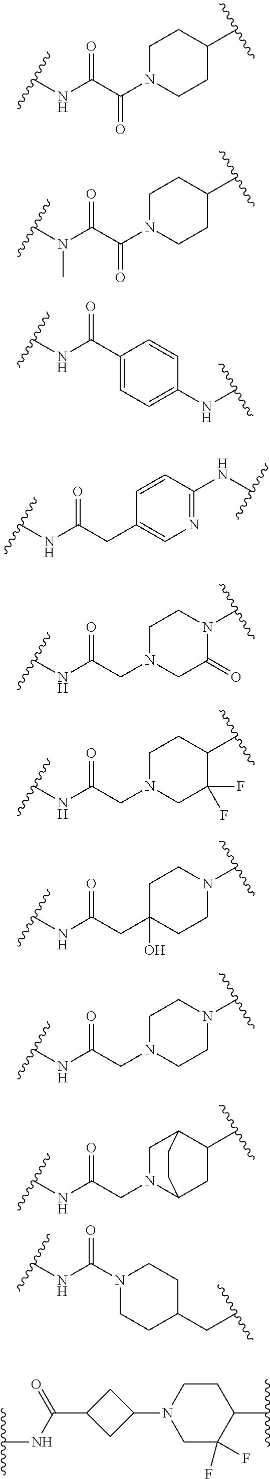

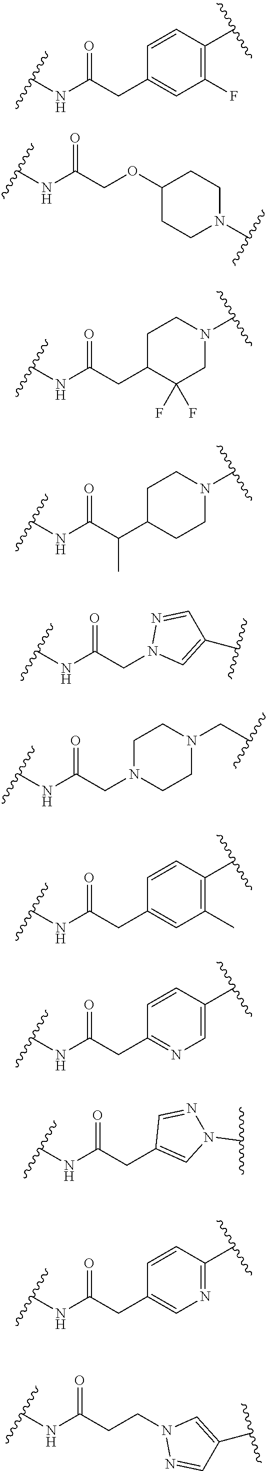

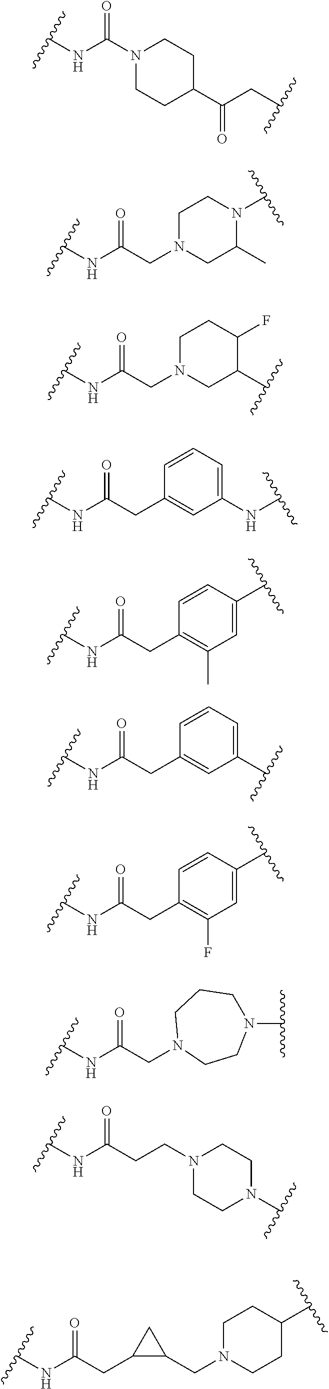

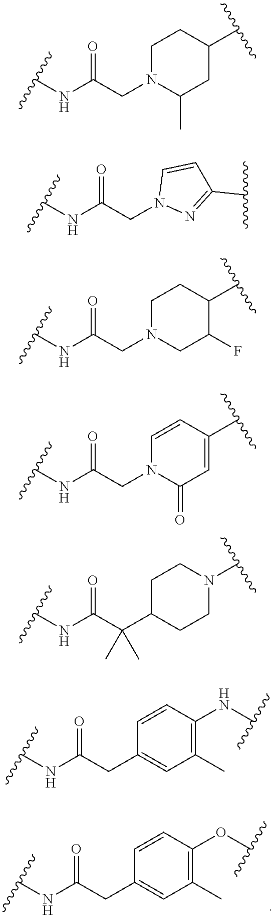

In some embodiments, L is selected from the group consisting of:

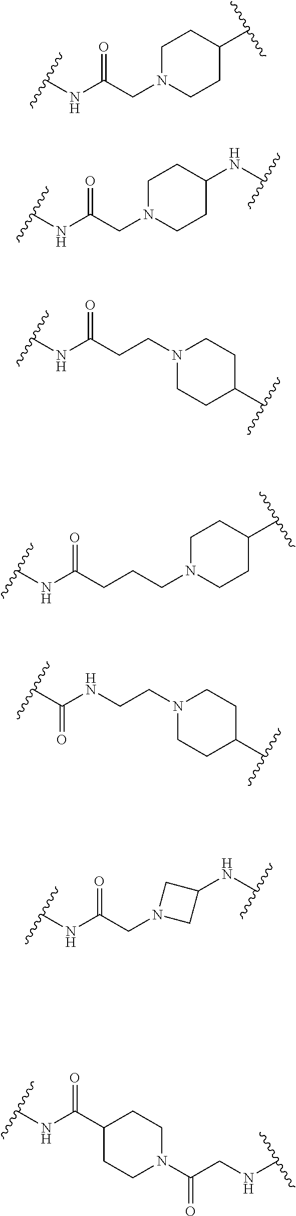

In some embodiments, L is selected from the group consisting of:

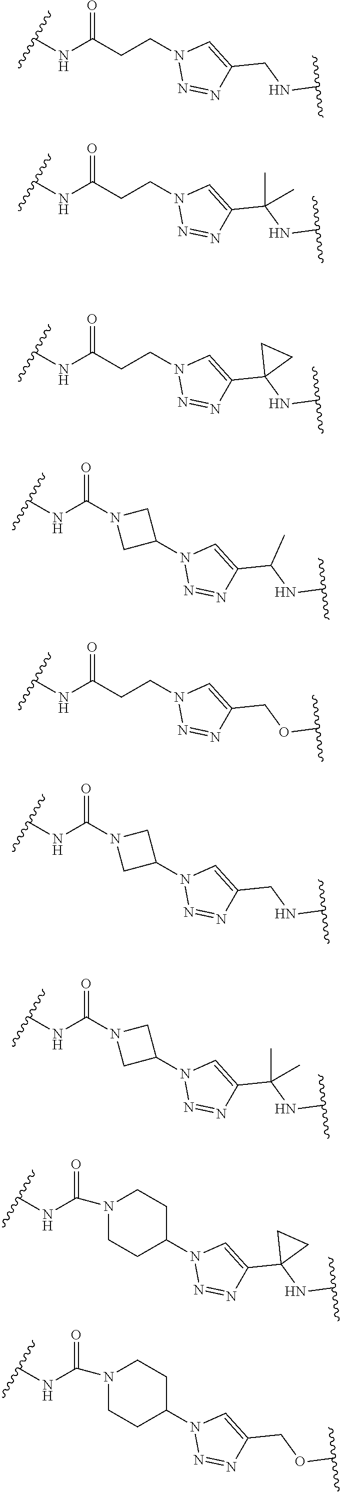

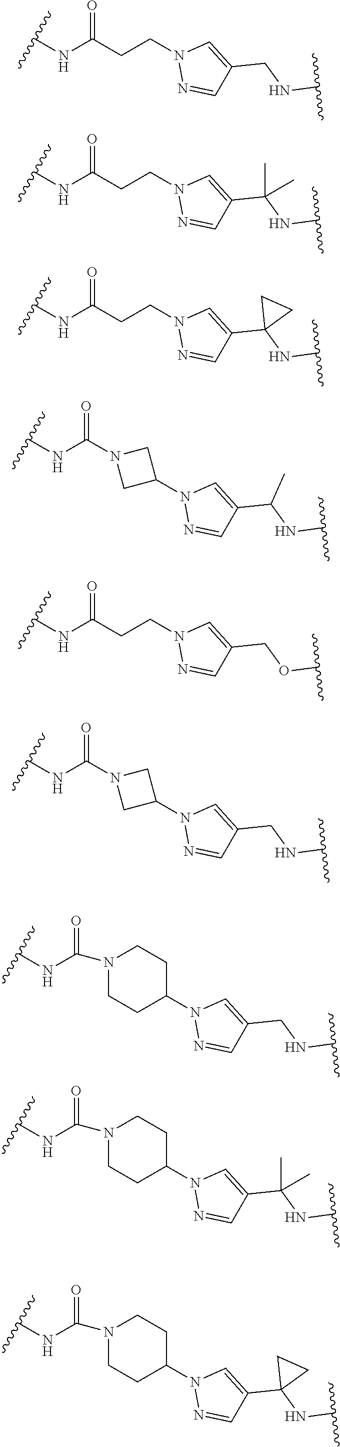

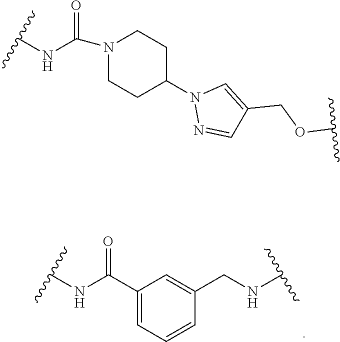 [SMILES: *NC(=O)N1CCC(n2cc(CO*)cn2)CC1 ; *NCc1cccc(C(=O)N*)c1]

In some embodiments, L is selected from the group consisting of:

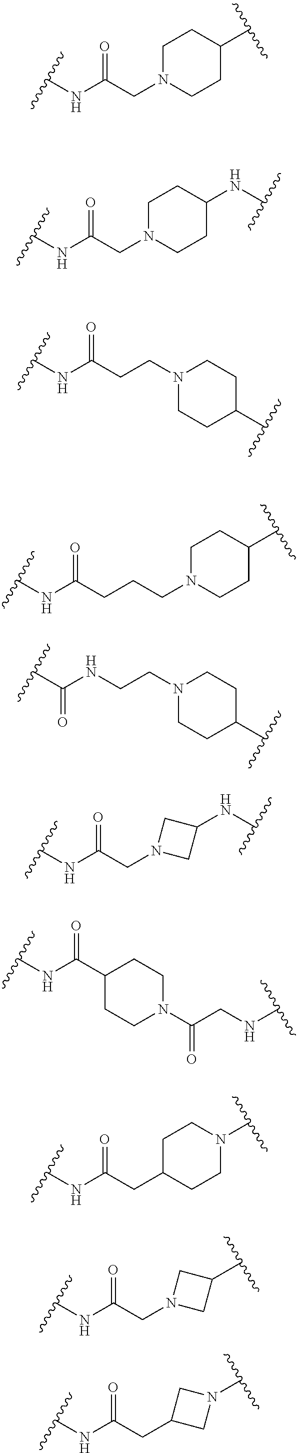

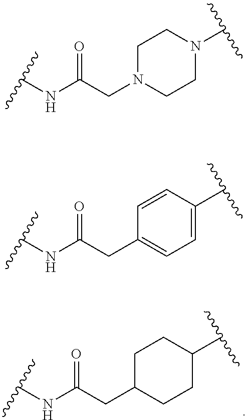 [SMILES: *NC(=O)CN1CCN(*)CC1 ; *NC(=O)Cc1ccc(*)cc1 ; *NC(=O)CC1CCC(*)CC1]

In some embodiments, L is selected from the group consisting of:

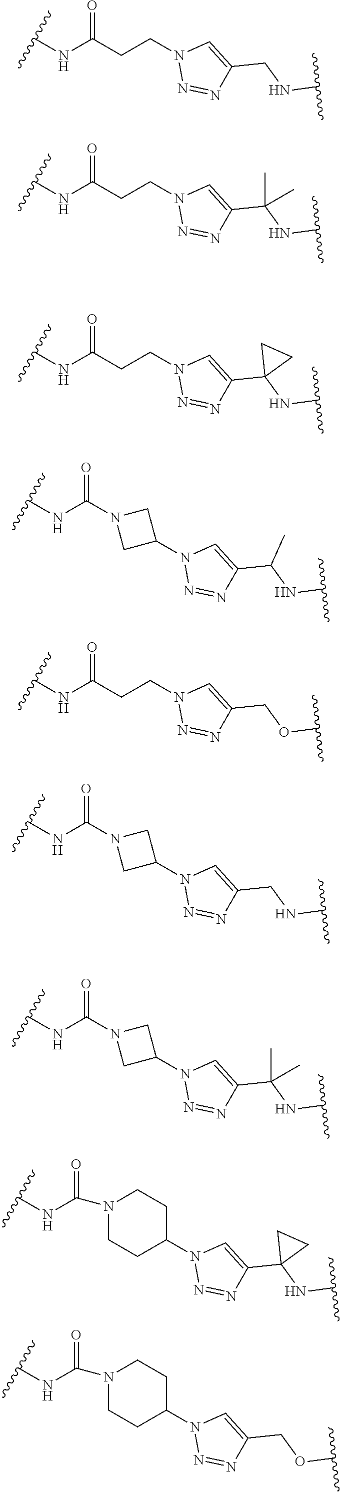

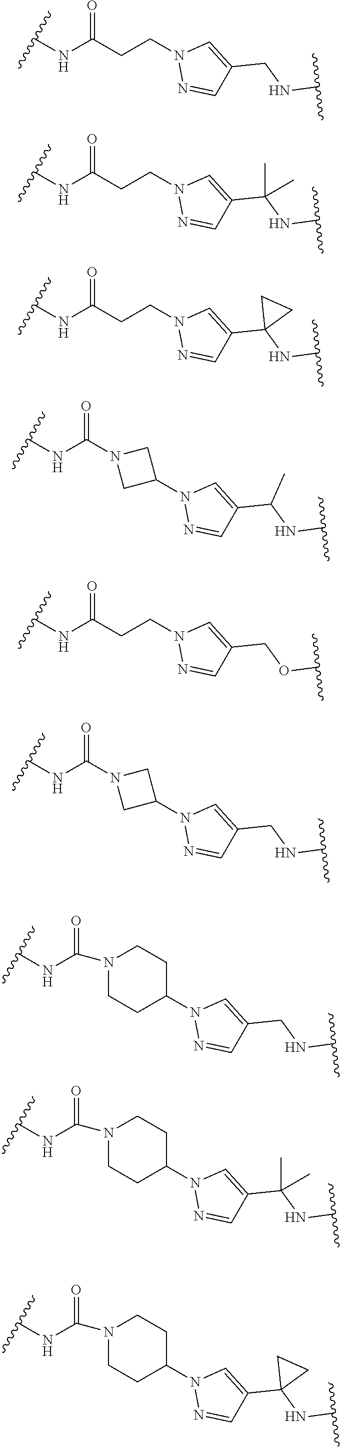

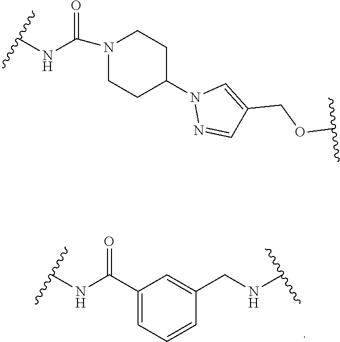 [SMILES: *NC(=O)N1CCC(n2cc(CO*)cn2)CC1 ; *NCc1cccc(C(=O)N*)c1]

In some embodiments, L is selected from the group consisting of:

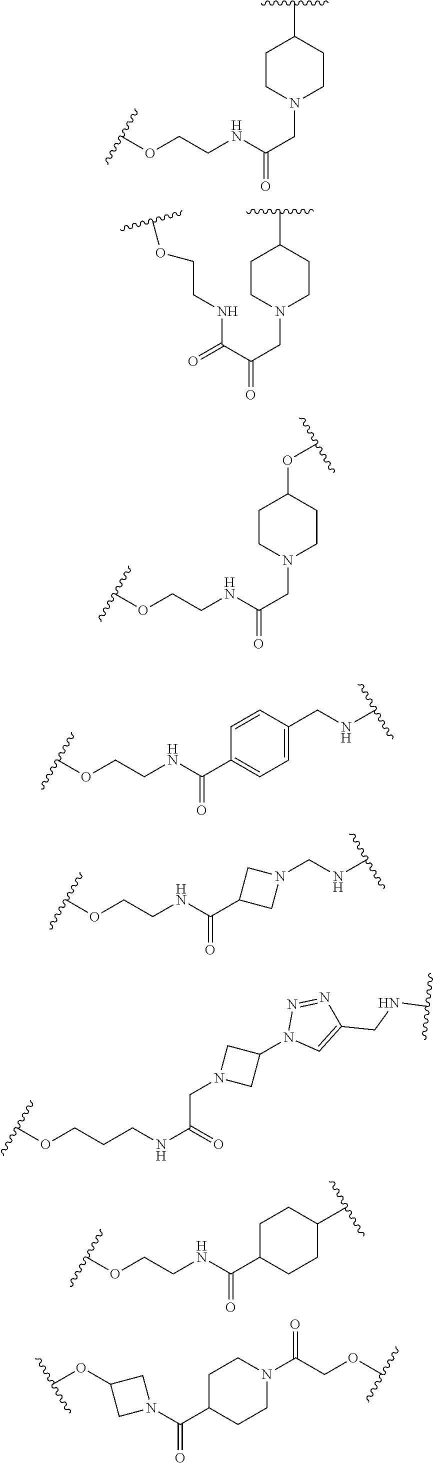

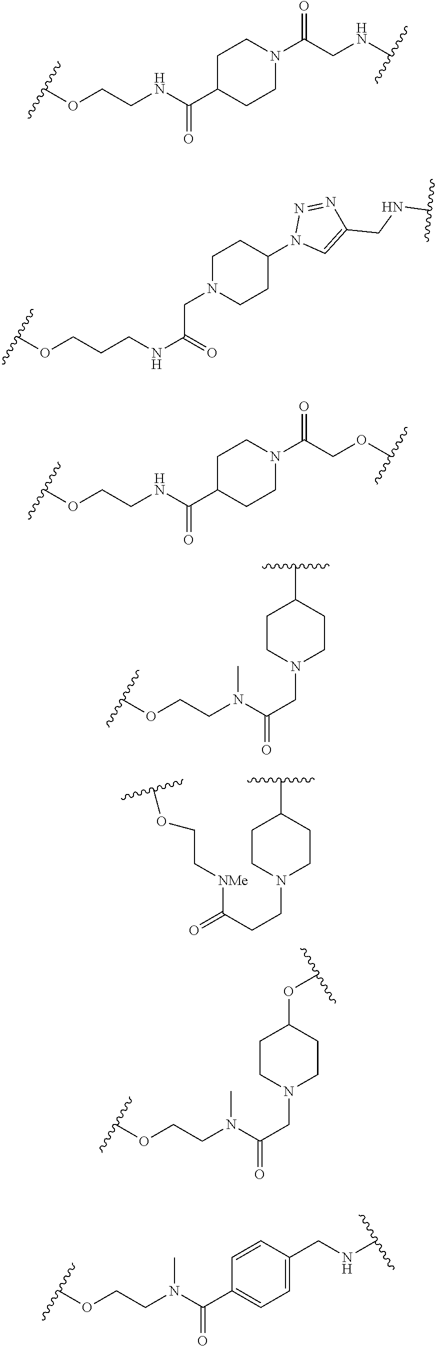

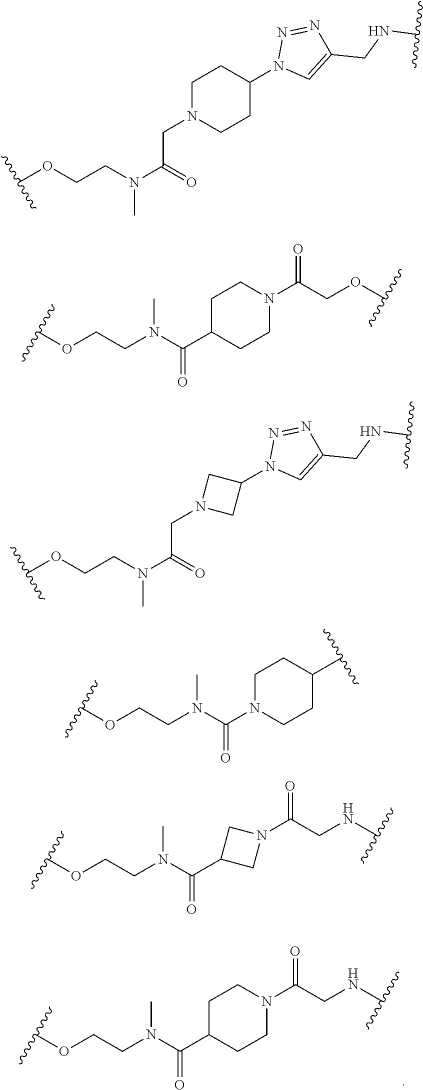 [SMILES: *NCc1cn(C2CCN(CC(=O)N(C)CCO*)CC2)nn1 ; *OCCN(C)C(=O)C1CCN(C(=O)CO*)CC1 ; *NCc1cn(C2CN(CC(=O)N(C)CCO*)C2)nn1 ; *OCCN(C)C(=O)N1CCC(*)CC1 ; *NCC(=O)N1CC(C(=O)N(C)CCO*)C1 ; *NCC(=O)N1CCC(C(=O)N(C)CCO*)CC1]

In some embodiments, L is selected from the group consisting of:

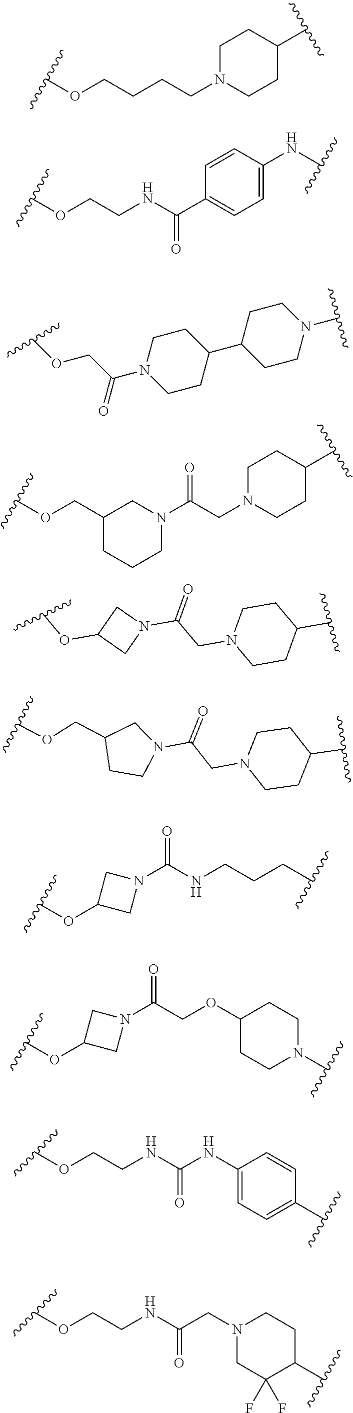

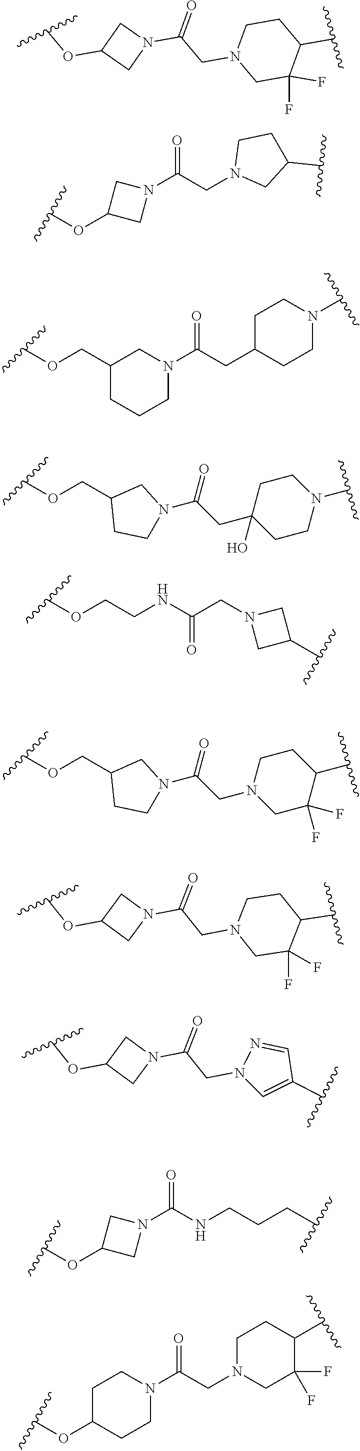

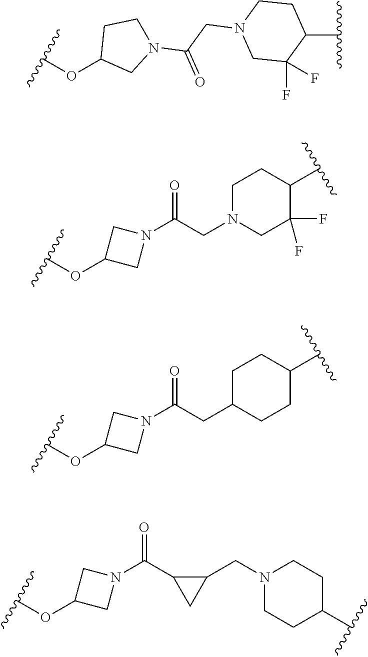 [SMILES: *OC1CCN(C(=O)CN2CCC(*)C(F)(F)C2)C1 ; *OC1CN(C(=O)CN2CCC(*)C(F)(F)C2)C1 ; *OC1CN(C(=O)CC2CCC(*)CC2)C1 ; *OC1CN(C(=O)C2CC2CN2CCC(*)CC2)C1]

In some embodiments, L is selected from the group consisting of:

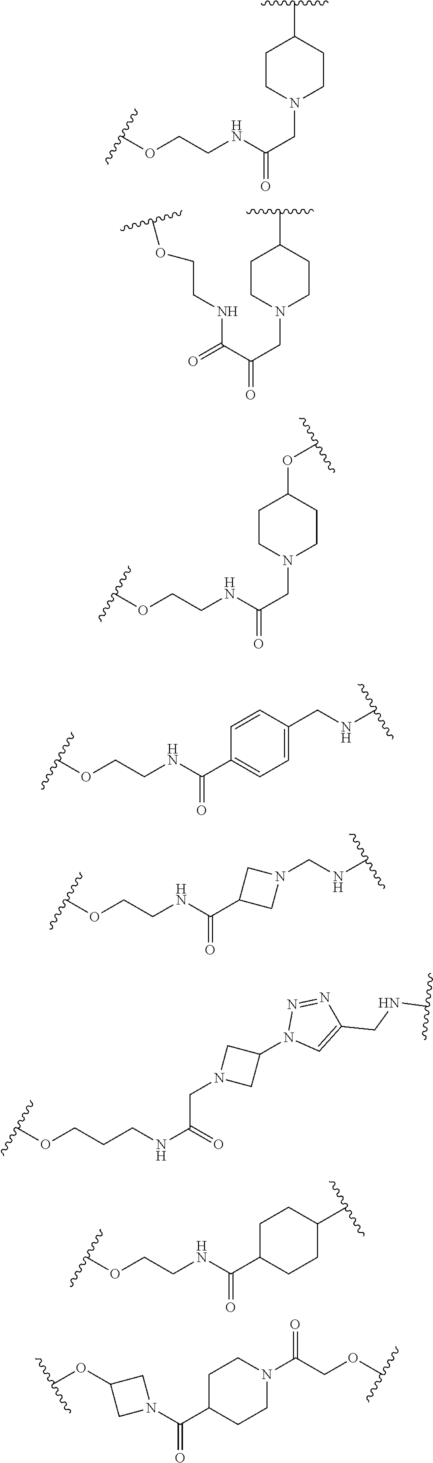

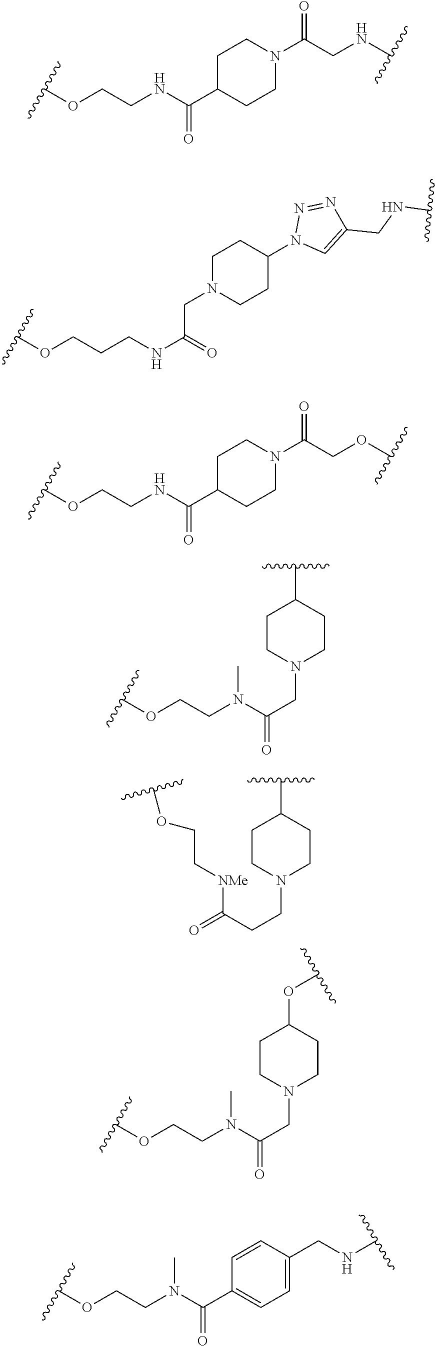

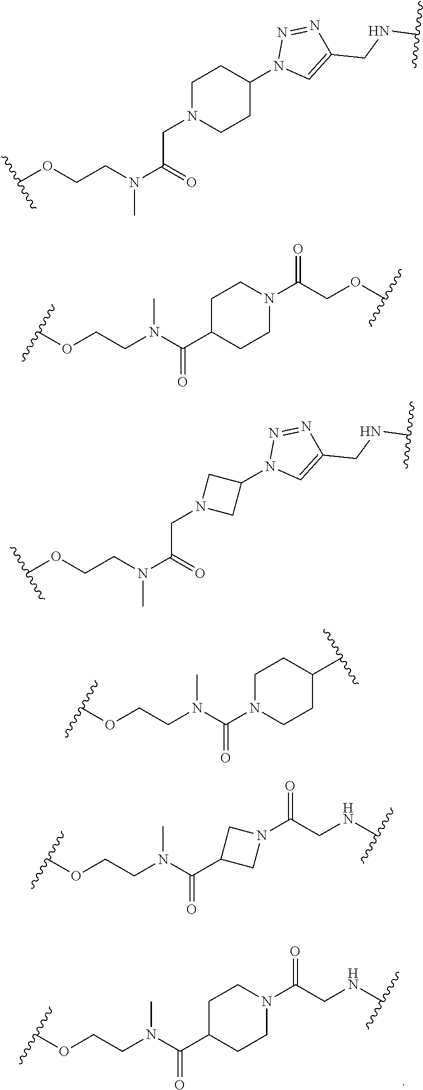 [SMILES: *NCc1cn(C2CCN(CC(=O)N(C)CCO*)CC2)nn1 ; *OCCN(C)C(=O)C1CCN(C(=O)CO*)CC1 ; *NCc1cn(C2CN(CC(=O)N(C)CCO*)C2)nn1 ; *OCCN(C)C(=O)N1CCC(*)CC1 ; *NCC(=O)N1CC(C(=O)N(C)CCO*)C1 ; *NCC(=O)N1CCC(C(=O)N(C)CCO*)CC1]

In some embodiments, L is selected from the group consisting of:

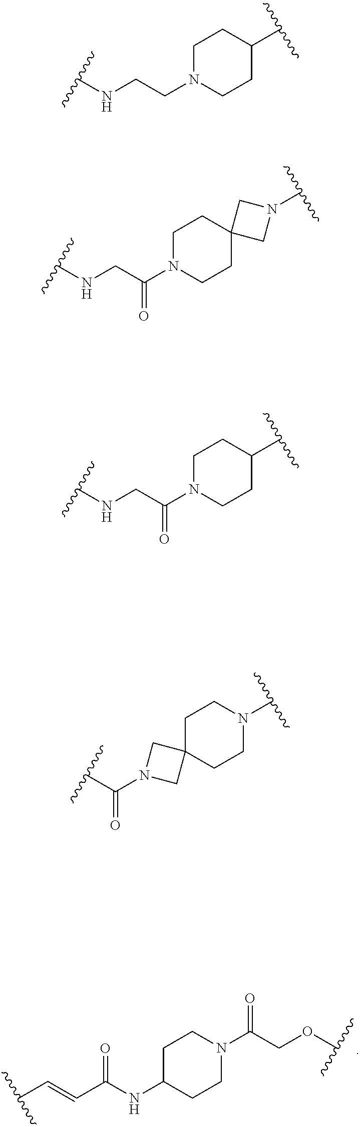 [SMILES: *NCCN1CCC(*)CC1 ; *NCC(=O)N1CCC2(CC1)CN(*)C2 ; *NCC(=O)N1CCC(*)CC1 ; *C(=O)N1CC2(CCN(*)CC2)C1 ; */C=C/C(=O)NC1CCN(C(=O)CO*)CC1]

Some embodiments provide a compound of Formula (I):

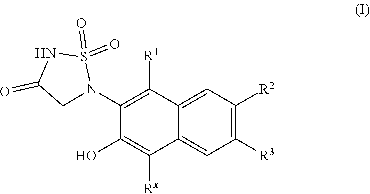

or a pharmaceutically acceptable salt thereof:
wherein:
R¹ is hydrogen or halogen;
R² is hydrogen, halogen, C1-C3 alkoxy, C3 cycloalkoxy, C1-C3 haloalkoxy, C3-C5 halocycloalkoxy, C1-C3 alkyl, C3 cycloalkyl, or -L-Z;
R³ is hydrogen, halogen, C1-C3 alkoxy, C3-C5 cycloalkoxy, C1-C3 haloalkoxy, C3-C5 halocycloalkoxy, C1-C3 alkyl, C3-C5 cycloalkyl, or -L-Z;
wherein one of R² and R³ is -L-Z and the other of R² and R³ is not-L-Z;
R^{x} is hydrogen or halogen;
L is selected from the group consisting of:

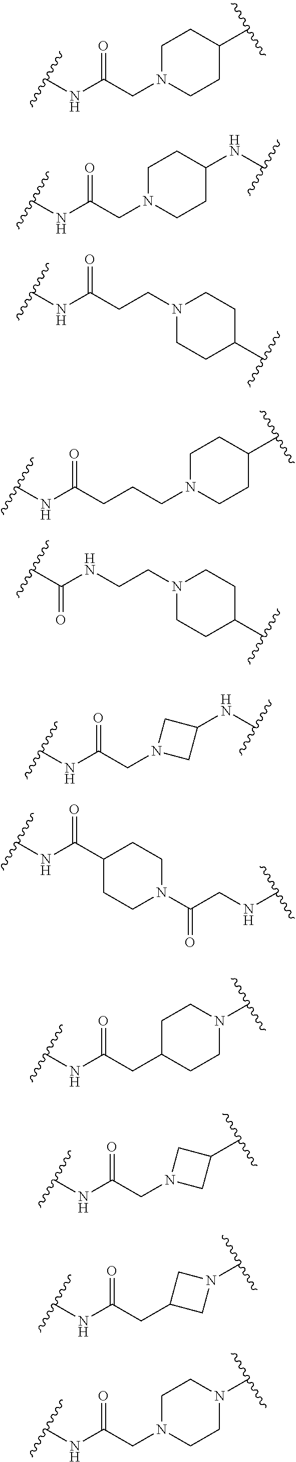

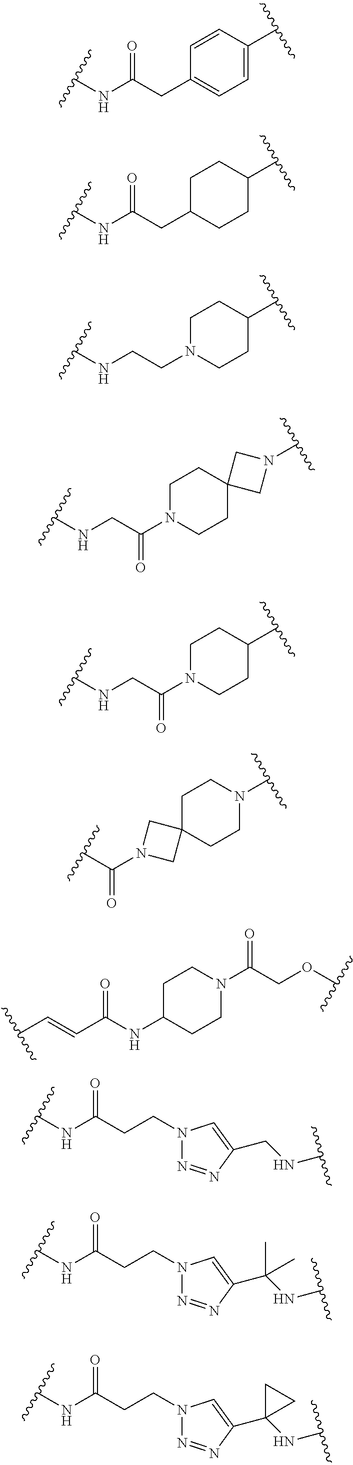

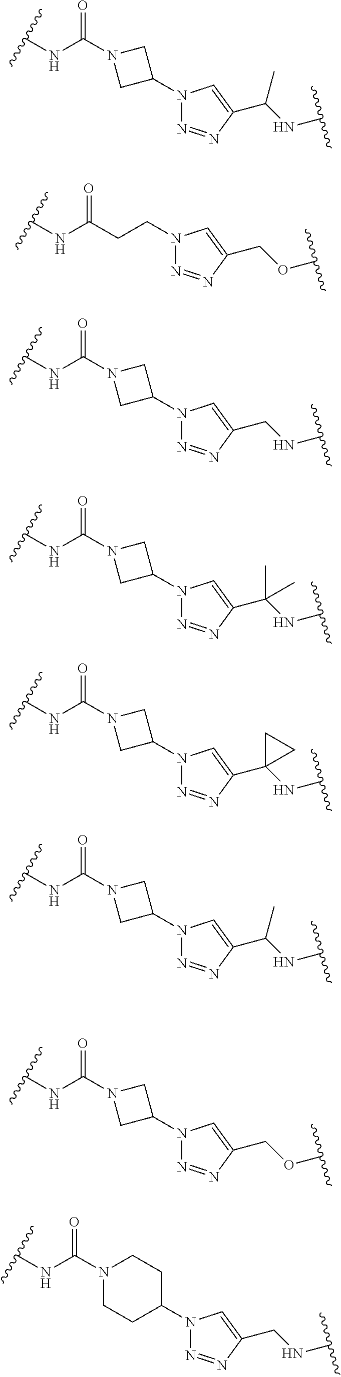

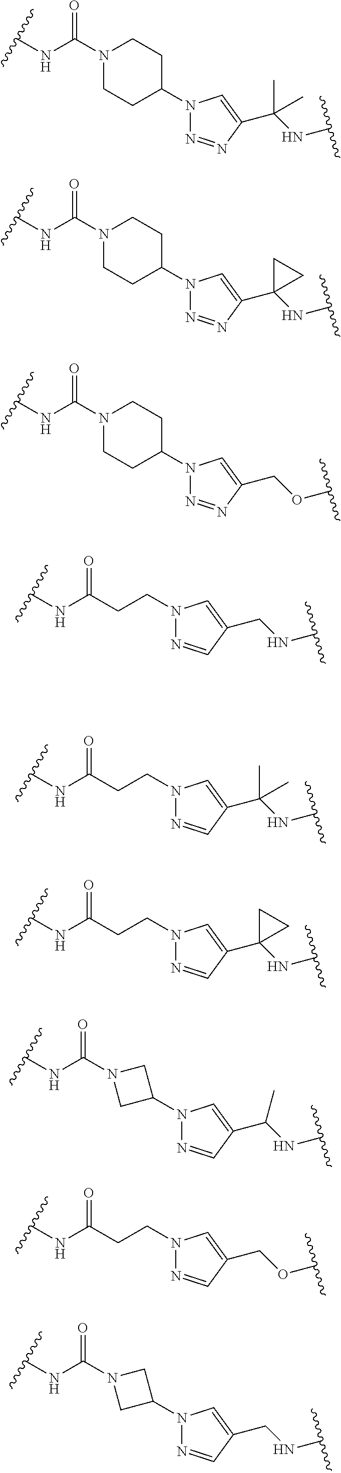

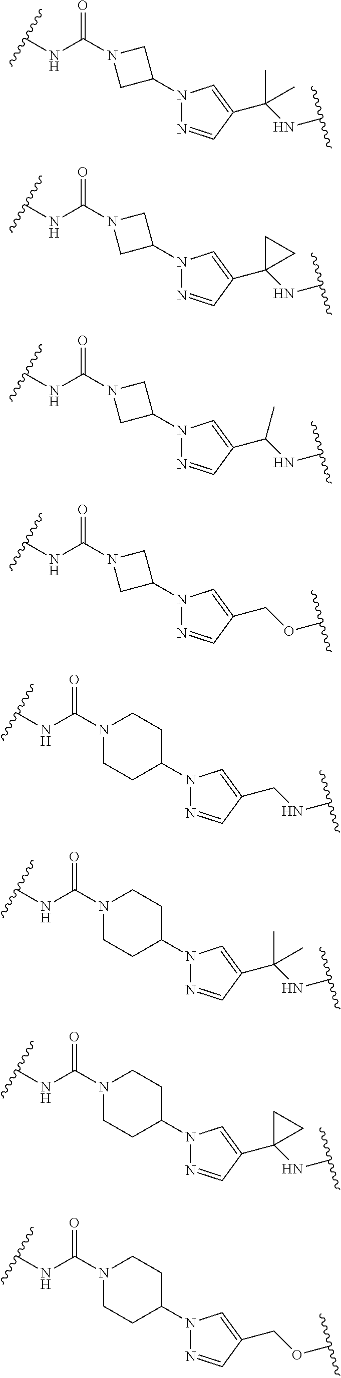

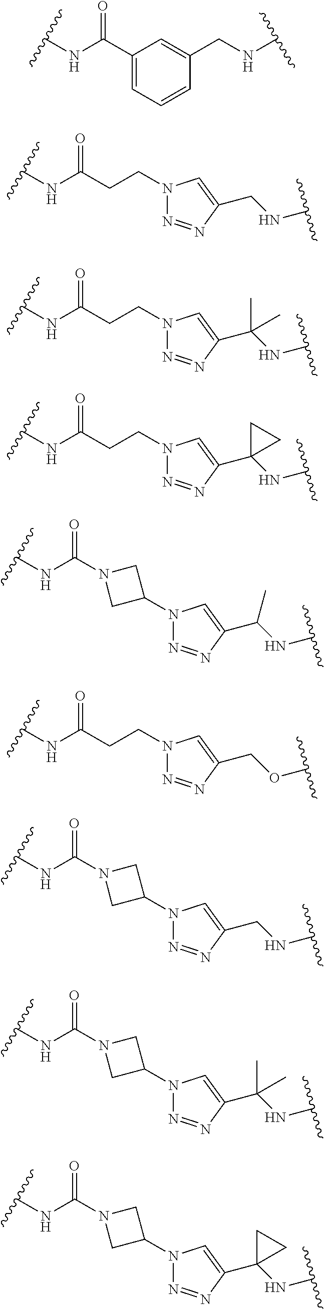

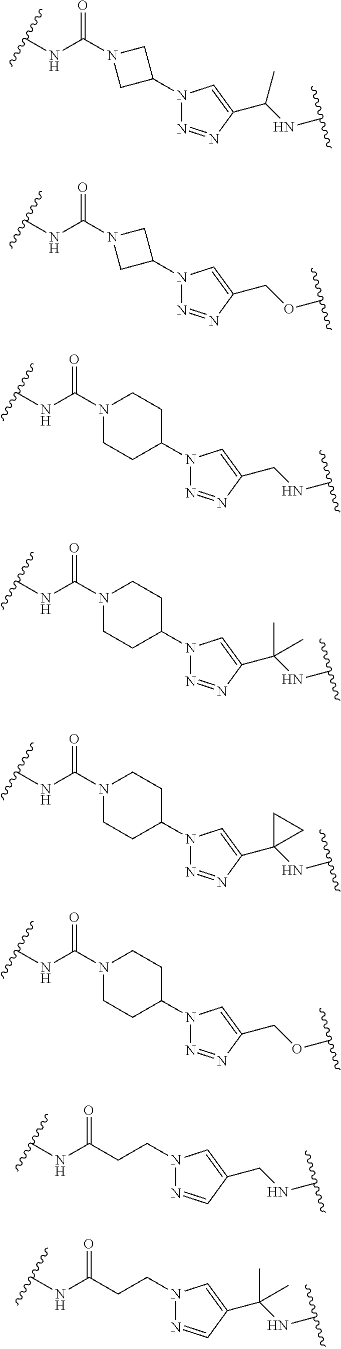

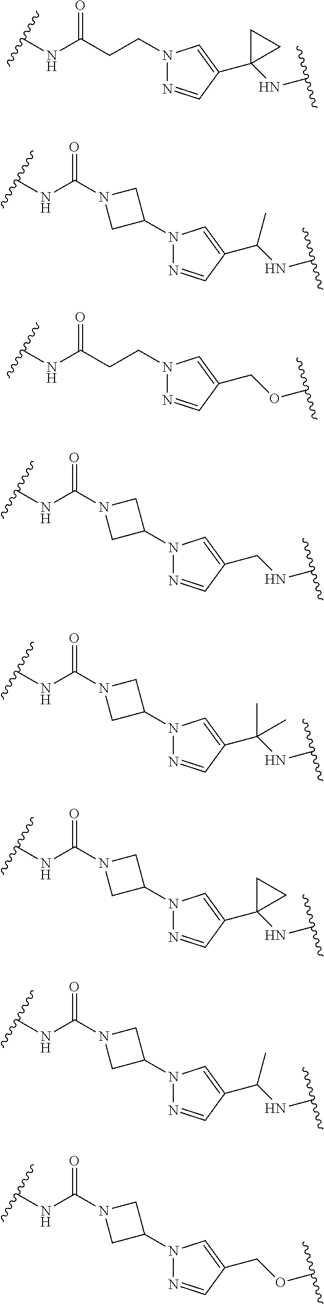

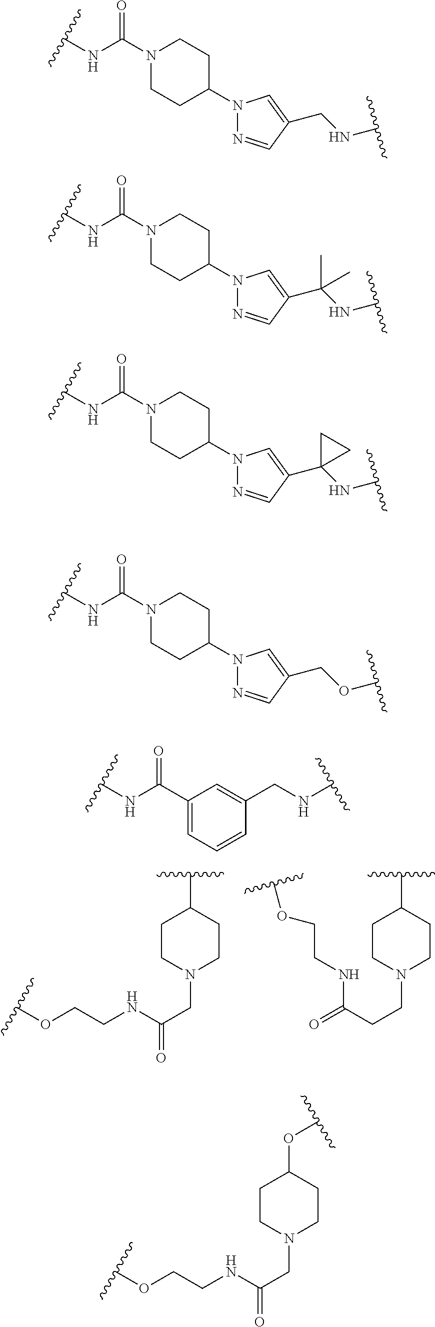

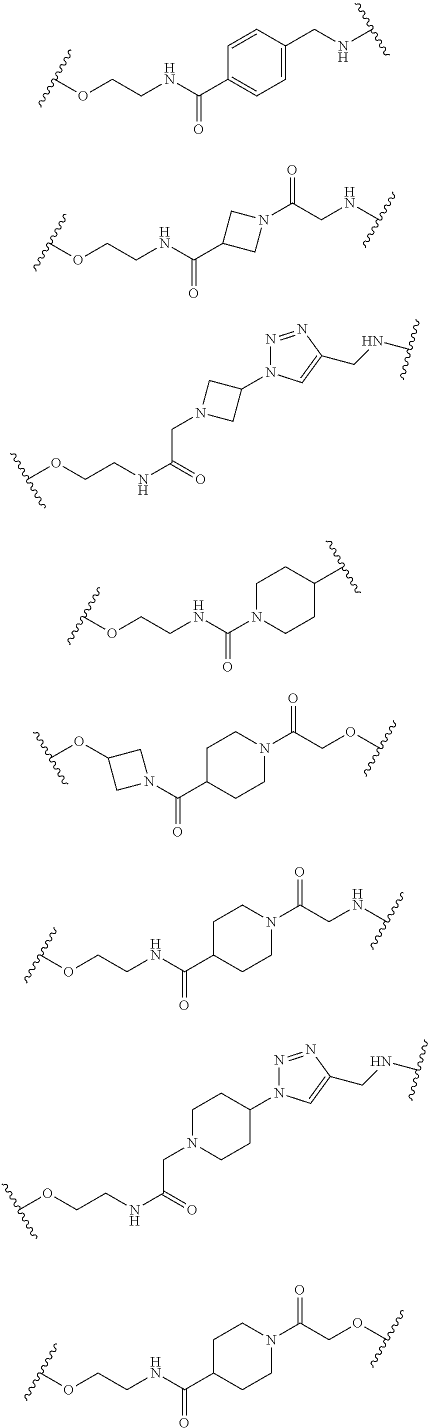

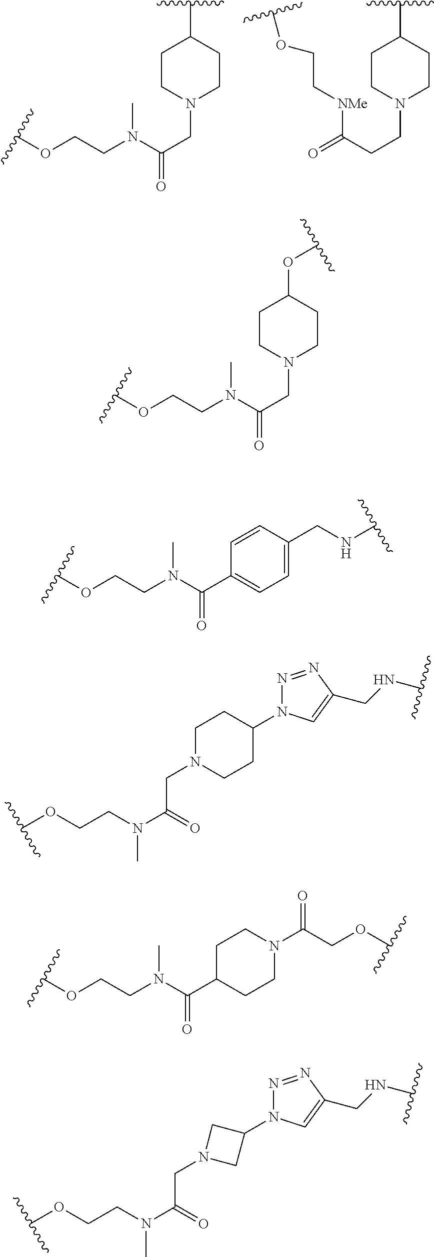

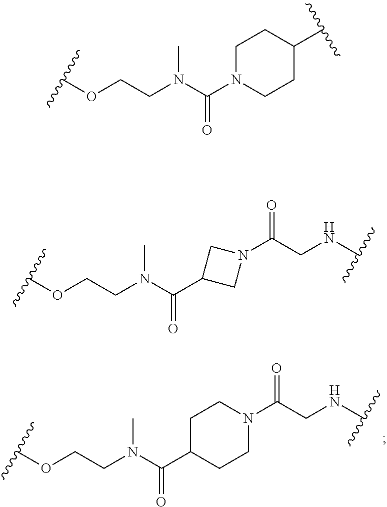 [SMILES: *OCCN(C)C(=O)N1CCC(*)CC1 ; *NCC(=O)N1CC(C(=O)N(C)CCO*)C1 ; *NCC(=O)N1CCC(C(=O)N(C)CCO*)CC1]

Z is selected from the group consisting of

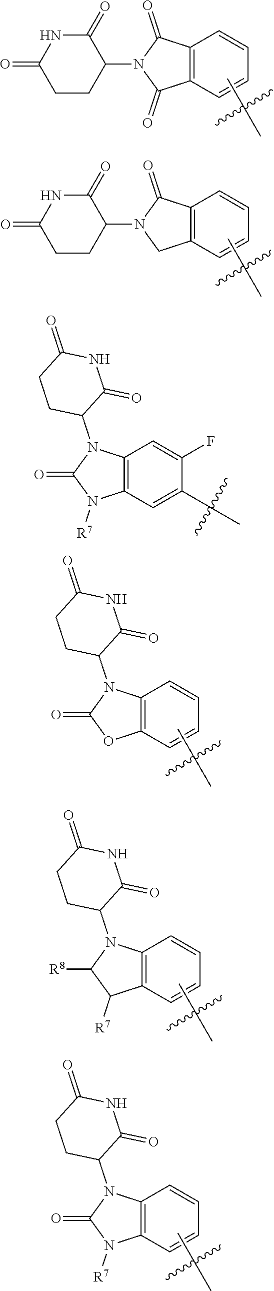

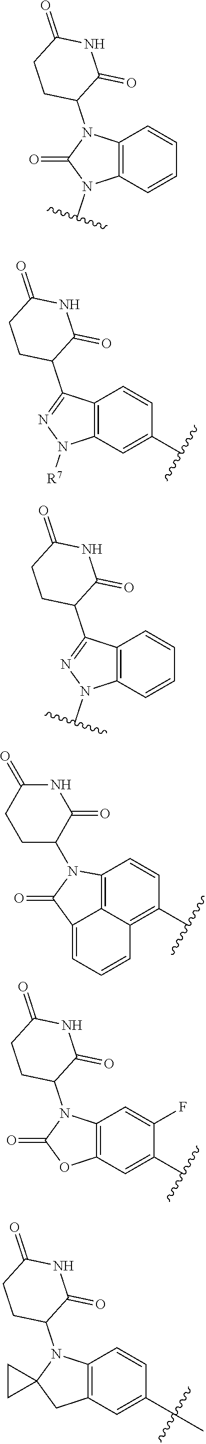

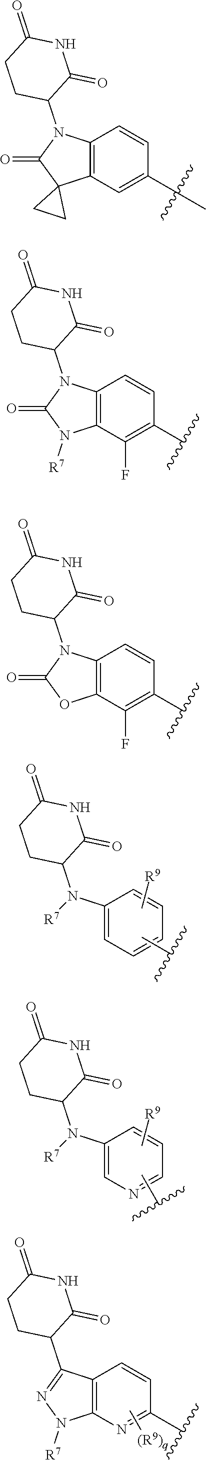

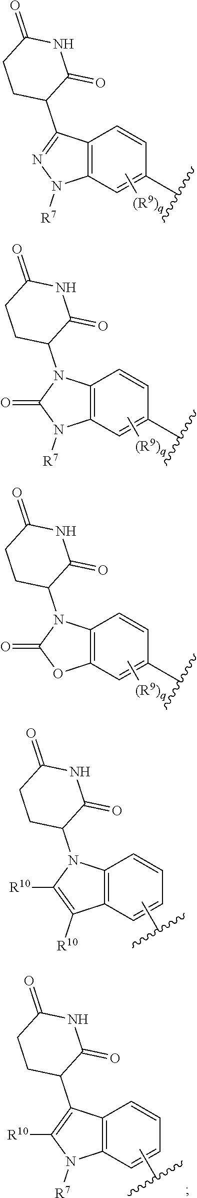

R⁷ is hydrogen, C1-C6 alkyl optionally substituted with one group selected from hydroxyl, cyano and C1-C6 alkoxy, C1-C6 haloalkyl, C3-C6 cycloalkyl, 4-6 membered heterocyclyl, —(CR^{A}R^{B})(4-12 membered heterocyclyl), or —(CR^{A}R^{B})(C3-C6 cycloalkyl);
R⁸ is hydrogen or C1-C6 alkyl; and
each R⁹ is halogen, cyano, C1-C6 alkyl, C1-C6 haloalkyl, C1-C6 alkoxy, C1-C5 cycloalkoxy, 5-10 membered heteroaryloxy, or phenoxy;
q is 0, 1, or 2; and
each R¹⁰ is independently hydrogen, halogen, cyano, C1-C6 alkyl, C3-C6 cycloalkyl, or C1-C6 haloalkyl.

In some embodiments, a compound of Formula (I) is selected from a compound set forth in Table 1.

TABLE 1
Exemplary compounds of the disclosure.

| Ex. | |
|---|---|
| No. | Structure |
| 1 | 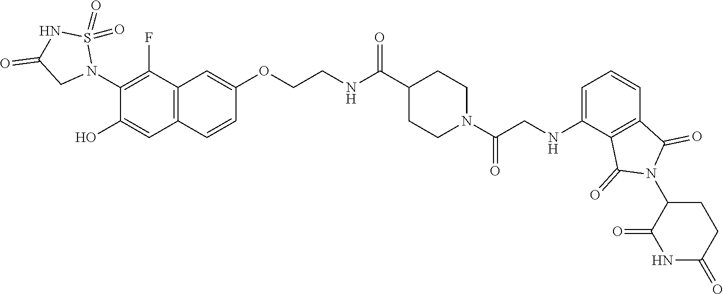 [SMILES: O=C1CCC(N2C(=O)c3cccc(NCC(=O)N4CCC(C(=O)NCCOc5ccc6cc(O)c(N7CC(=O)NS7(=O)=O)c(F)c6c5)CC4)c3C2=O)C(=O)N1] |
| 2 | 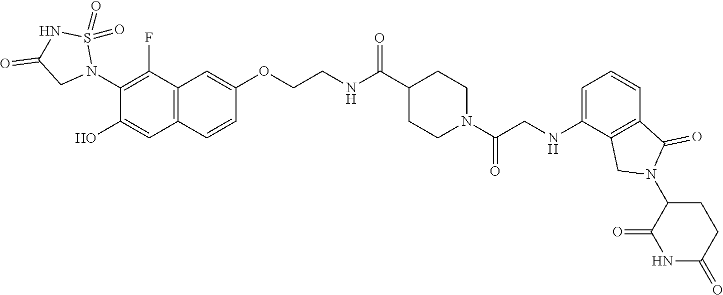 [SMILES: O=C1CCC(N2Cc3c(NCC(=O)N4CCC(C(=O)NCCOc5ccc6cc(O)c(N7CC(=O)NS7(=O)=O)c(F)c6c5)CC4)cccc3C2=O)C(=O)N1] |
| 3 | 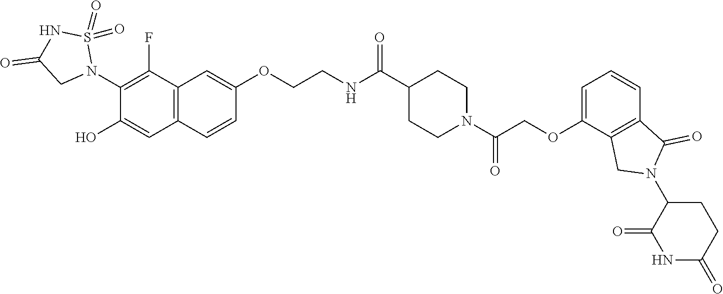 [SMILES: O=C1CCC(N2Cc3c(OCC(=O)N4CCC(C(=O)NCCOc5ccc6cc(O)c(N7CC(=O)NS7(=O)=O)c(F)c6c5)CC4)cccc3C2=O)C(=O)N1] |
| 4 | 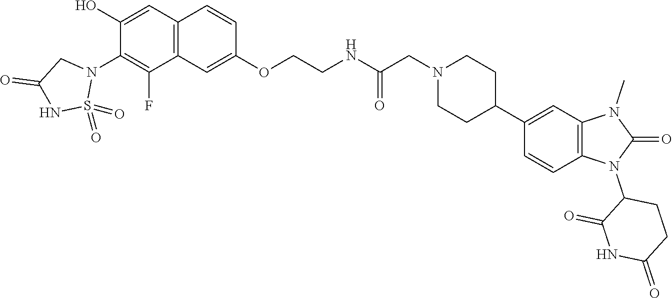 [SMILES: Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21] |
| 5 | 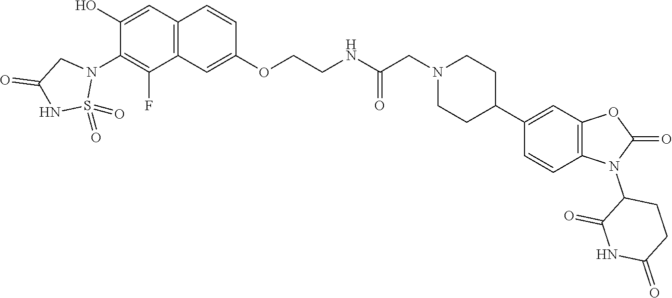 [SMILES: O=C(CN1CCC(c2ccc3c(c2)oc(=O)n3C2CCC(=O)NC2=O)CC1)NCCOc1ccc2cc(O)c(N3CC(=O)NS3(=O)=O)c(F)c2c1] |
| 6 | 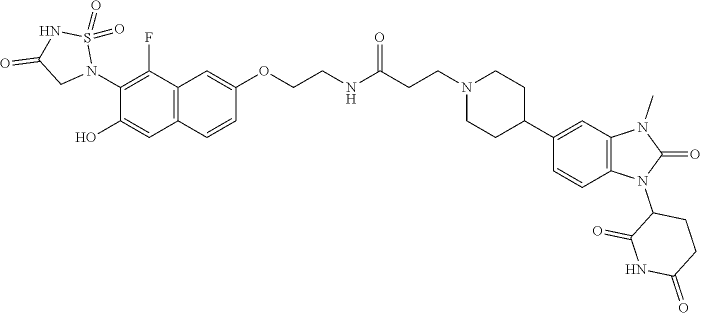 [SMILES: Cn1c(=O)n(C2CCC(=O)NC2=O)c2ccc(C3CCN(CCC(=O)NCCOc4ccc5cc(O)c(N6CC(=O)NS6(=O)=O)c(F)c5c4)CC3)cc21] |
| 7 | 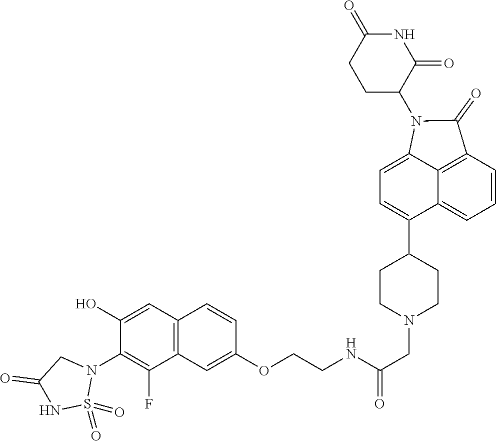 [SMILES: O=C(CN1CCC(c2ccc3c4c(cccc24)C(=O)N3C2CCC(=O)NC2=O)CC1)NCCOc1ccc2cc(O)c(N3CC(=O)NS3(=O)=O)c(F)c2c1] |
| 8 | 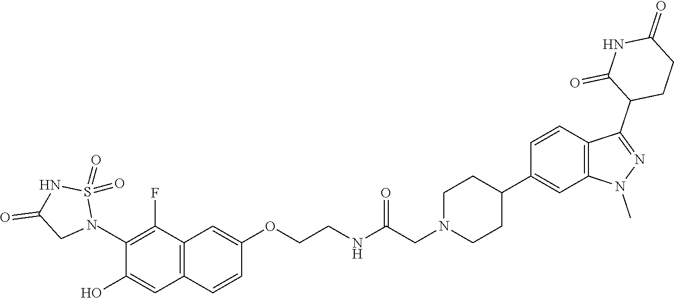 |
| 9 | 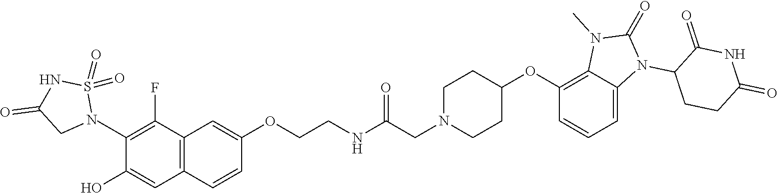 |
| 10 | 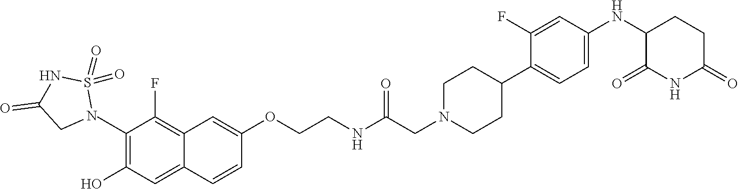 |
| 11 | 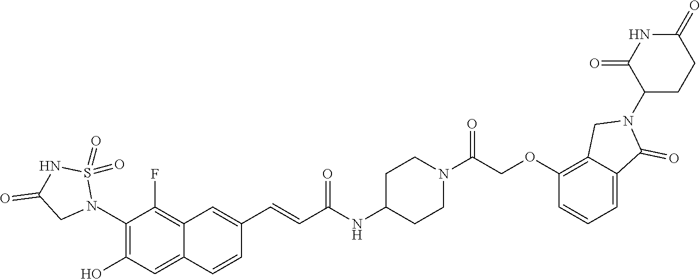 |
| 12 | 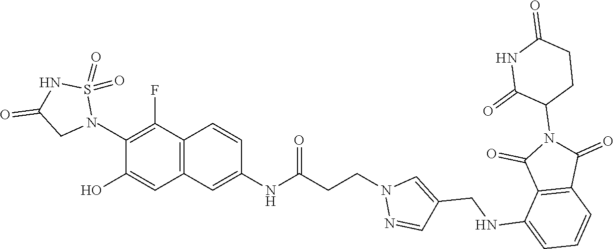 |
| 13 | 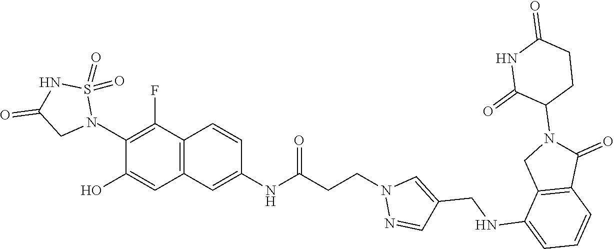 |
| 14 | 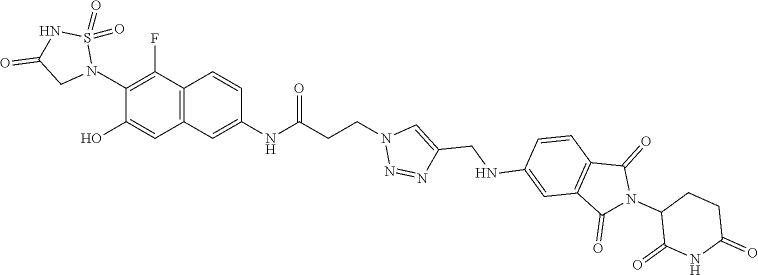 |
| 15 | 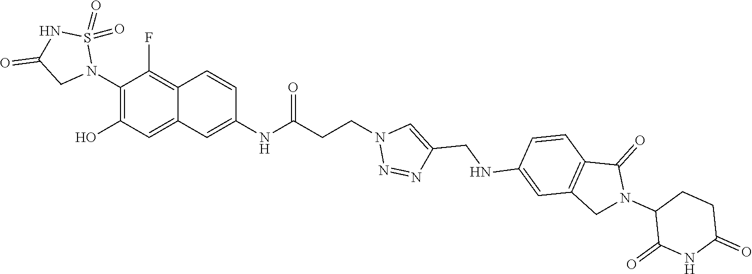 [SMILES: O=C1CCC(N2Cc3cc(NCc4cn(CCC(=O)Nc5ccc6c(F)c(N7CC(=O)NS7(=O)=O)c(O)cc6c5)nn4)ccc3C2=O)C(=O)N1] |
| 16 | 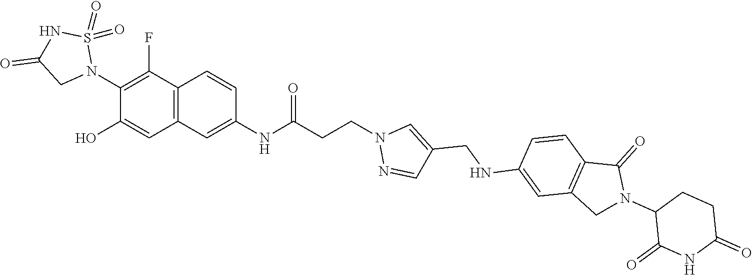 [SMILES: O=C1CCC(N2Cc3cc(NCc4cnn(CCC(=O)Nc5ccc6c(F)c(N7CC(=O)NS7(=O)=O)c(O)cc6c5)c4)ccc3C2=O)C(=O)N1] |
| 17 | 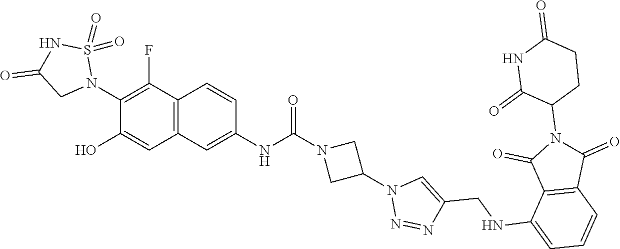 [SMILES: O=C1CCC(N2C(=O)c3cccc(NCc4cn(C5CN(C(=O)Nc6ccc7c(F)c(N8CC(=O)NS8(=O)=O)c(O)cc7c6)C5)nn4)c3C2=O)C(=O)N1] |
| 18 | 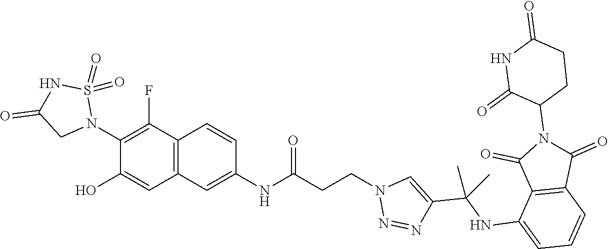 [SMILES: CC(C)(Nc1cccc2c1C(=O)N(C1CCC(=O)NC1=O)C2=O)c1cn(CCC(=O)Nc2ccc3c(F)c(N4CC(=O)NS4(=O)=O)c(O)cc3c2)nn1] |
| 19 | 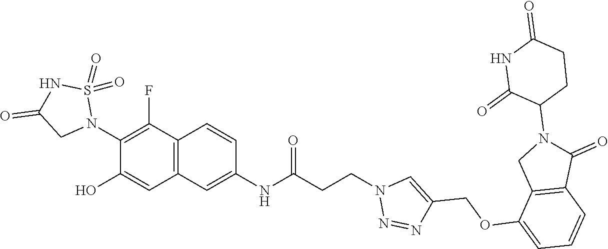 [SMILES: O=C1CCC(N2Cc3c(OCc4cn(CCC(=O)Nc5ccc6c(F)c(N7CC(=O)NS7(=O)=O)c(O)cc6c5)nn4)cccc3C2=O)C(=O)N1] |
| 20 | 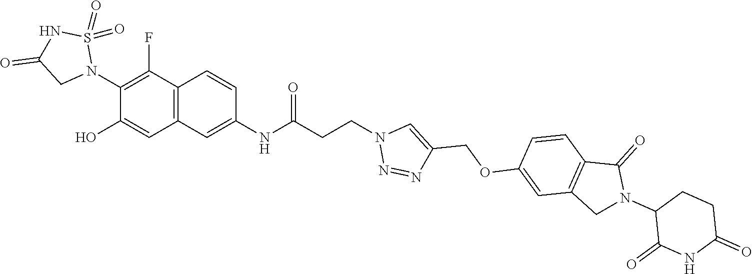 [SMILES: O=C1CCC(N2Cc3cc(OCc4cn(CCC(=O)Nc5ccc6c(F)c(N7CC(=O)NS7(=O)=O)c(O)cc6c5)nn4)ccc3C2=O)C(=O)N1] |
| 21 | 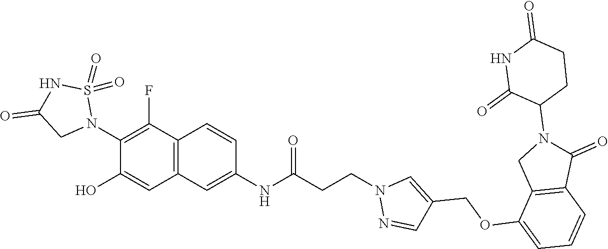 [SMILES: O=C1CCC(N2Cc3c(OCc4cnn(CCC(=O)Nc5ccc6c(F)c(N7CC(=O)NS7(=O)=O)c(O)cc6c5)c4)cccc3C2=O)C(=O)N1] |
| 22 | 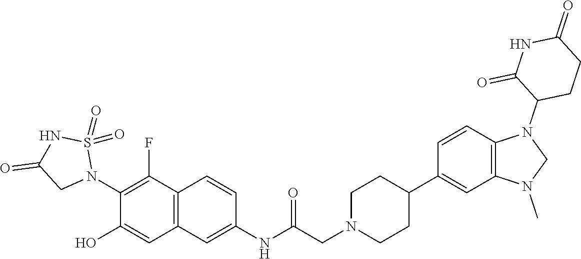 [SMILES: CN1CN(C2CCC(=O)NC2=O)c2ccc(C3CCN(CC(=O)Nc4ccc5c(F)c(N6CC(=O)NS6(=O)=O)c(O)cc5c4)CC3)cc21] |
| 23 | 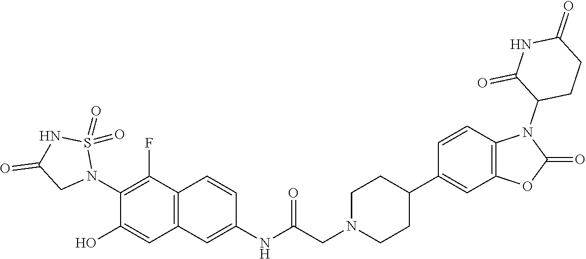 [SMILES: O=C1CCC(n2c(=O)oc3cc(C4CCN(CC(=O)Nc5ccc6c(F)c(N7CC(=O)NS7(=O)=O)c(O)cc6c5)CC4)ccc32)C(=O)N1] |
| 24 | 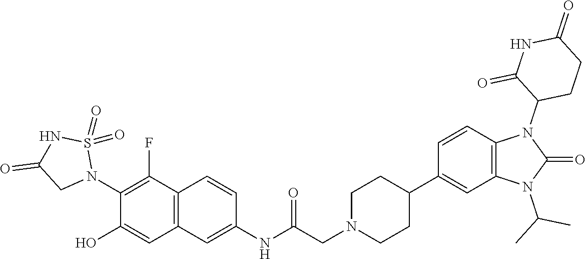 [SMILES: CC(C)n1c(=O)n(C2CCC(=O)NC2=O)c2ccc(C3CCN(CC(=O)Nc4ccc5c(F)c(N6CC(=O)NS6(=O)=O)c(O)cc5c4)CC3)cc21] |
| 25 | 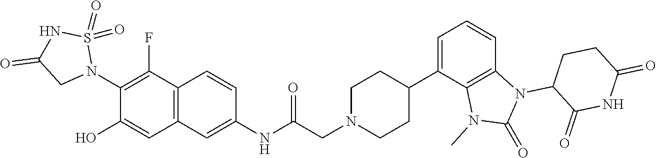 [SMILES: Cn1c(=O)n(C2CCC(=O)NC2=O)c2cccc(C3CCN(CC(=O)Nc4ccc5c(F)c(N6CC(=O)NS6(=O)=O)c(O)cc5c4)CC3)c21] |
| 26 | 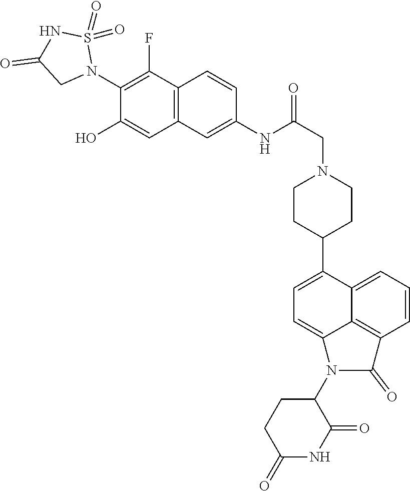 [SMILES: O=C1CCC(N2C(=O)c3cccc4c(C5CCN(CC(=O)Nc6ccc7c(F)c(N8CC(=O)NS8(=O)=O)c(O)cc7c6)CC5)ccc2c34)C(=O)N1] |
| 27 | 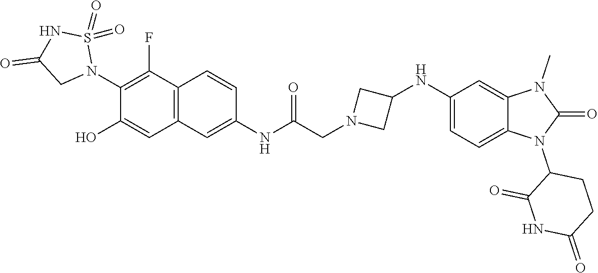 [SMILES: Cn1c(=O)n(C2CCC(=O)NC2=O)c2ccc(NC3CN(CC(=O)Nc4ccc5c(F)c(N6CC(=O)NS6(=O)=O)c(O)cc5c4)C3)cc21] |
| 28 | 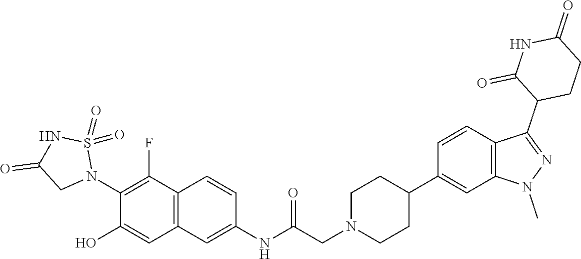 [SMILES: Cn1nc(C2CCC(=O)NC2=O)c2ccc(C3CCN(CC(=O)Nc4ccc5c(F)c(N6CC(=O)NS6(=O)=O)c(O)cc5c4)CC3)cc21] |
| 29 | 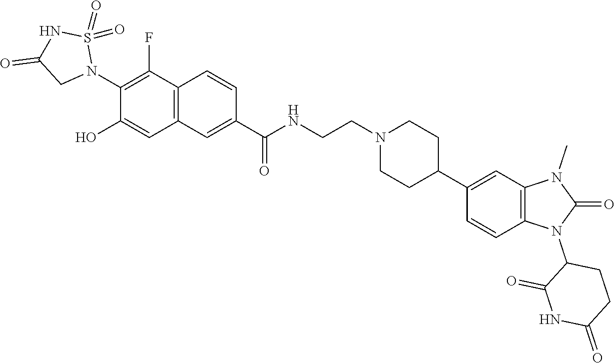 [SMILES: Cn1c(=O)n(C2CCC(=O)NC2=O)c2ccc(C3CCN(CCNC(=O)c4ccc5c(F)c(N6CC(=O)NS6(=O)=O)c(O)cc5c4)CC3)cc21] |
| 30 | 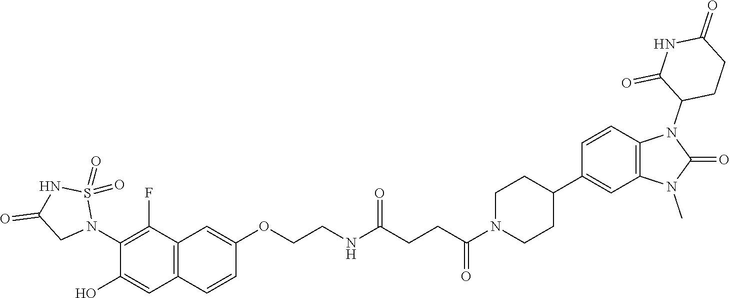 [SMILES: Cn1c(=O)n(C2CCC(=O)NC2=O)c2ccc(C3CCN(C(=O)CCC(=O)NCCOc4ccc5cc(O)c(N6CC(=O)NS6(=O)=O)c(F)c5c4)CC3)cc21] |
| 31 | 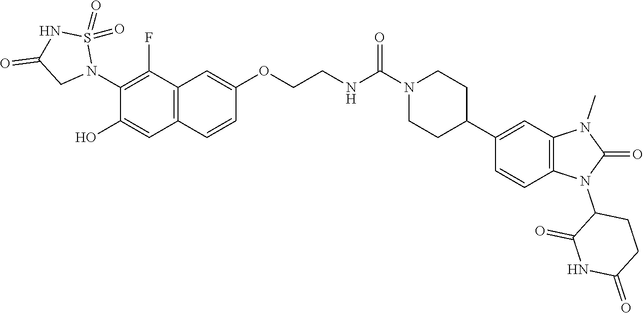 [SMILES: Cn1c(=O)n(C2CCC(=O)NC2=O)c2ccc(C3CCN(C(=O)NCCOc4ccc5cc(O)c(N6CC(=O)NS6(=O)=O)c(F)c5c4)CC3)cc21] |
| 32 | 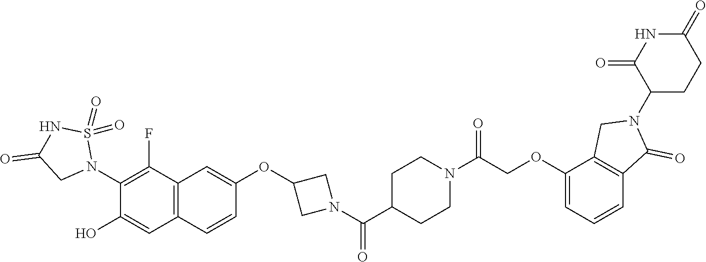 [SMILES: O=C1CCC(N2Cc3c(OCC(=O)N4CCC(C(=O)N5CC(Oc6ccc7cc(O)c(N8CC(=O)NS8(=O)=O)c(F)c7c6)C5)CC4)cccc3C2=O)C(=O)N1] |
| 33 | 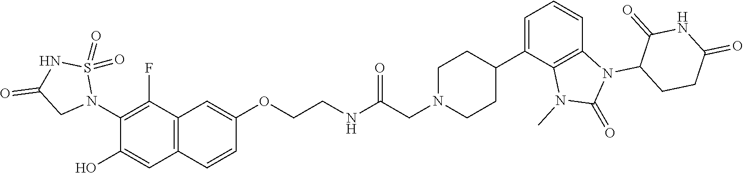 [SMILES: Cn1c(=O)n(C2CCC(=O)NC2=O)c2cccc(C3CCN(CC(=O)NCCOc4ccc5cc(O)c(N6CC(=O)NS6(=O)=O)c(F)c5c4)CC3)c21] |
| 34 | 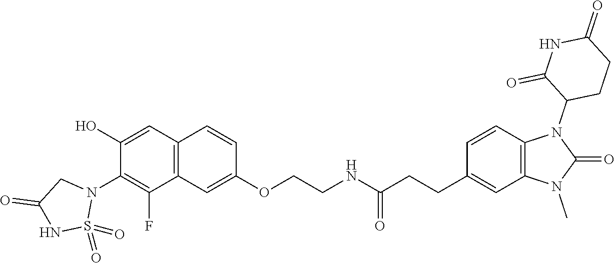 [SMILES: Cn1c(=O)n(C2CCC(=O)NC2=O)c2ccc(CCC(=O)NCCOc3ccc4cc(O)c(N5CC(=O)NS5(=O)=O)c(F)c4c3)cc21] |
| 35 | 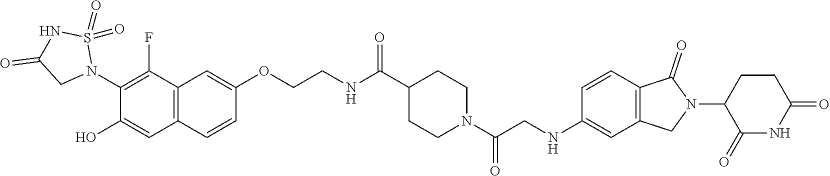 [SMILES: O=C1CCC(N2Cc3cc(NCC(=O)N4CCC(C(=O)NCCOc5ccc6cc(O)c(N7CC(=O)NS7(=O)=O)c(F)c6c5)CC4)ccc3C2=O)C(=O)N1] |
| 36 | 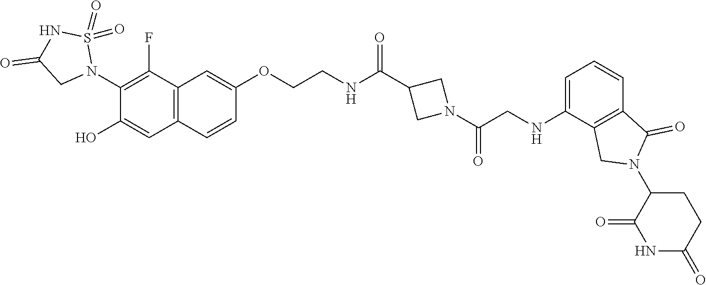 [SMILES: O=C1CCC(N2Cc3c(NCC(=O)N4CC(C(=O)NCCOc5ccc6cc(O)c(N7CC(=O)NS7(=O)=O)c(F)c6c5)C4)cccc3C2=O)C(=O)N1] |
| 37 | 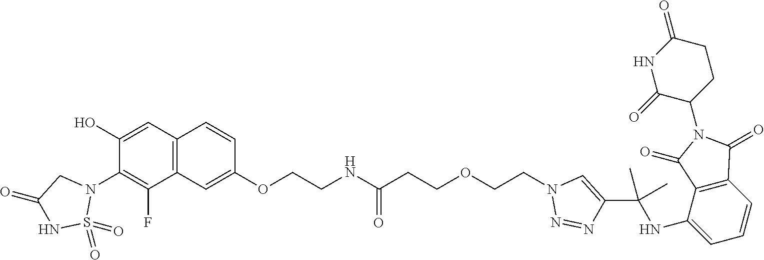 [SMILES: CC(C)(Nc1cccc2c1C(=O)N(C1CCC(=O)NC1=O)C2=O)c1cn(CCOCCC(=O)NCCOc2ccc3cc(O)c(N4CC(=O)NS4(=O)=O)c(F)c3c2)nn1] |
| 38 | 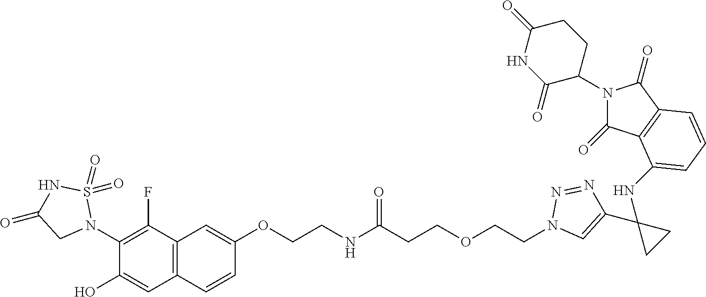 [SMILES: O=C(CCOCCn1cc(C2(Nc3cccc4c3C(=O)N(C3CCC(=O)NC3=O)C4=O)CC2)nn1)NCCOc1ccc2cc(O)c(N3CC(=O)NS3(=O)=O)c(F)c2c1] |
| 39 | 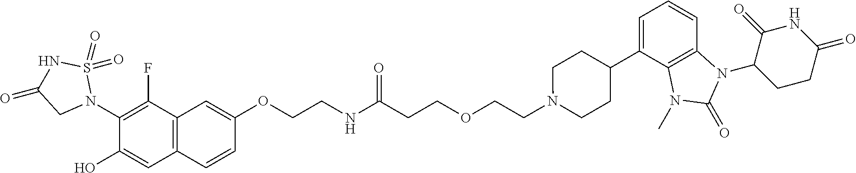 [SMILES: Cn1c(=O)n(C2CCC(=O)NC2=O)c2cccc(C3CCN(CCOCCC(=O)NCCOc4ccc5cc(O)c(N6CC(=O)NS6(=O)=O)c(F)c5c4)CC3)c21] |
| 40 | 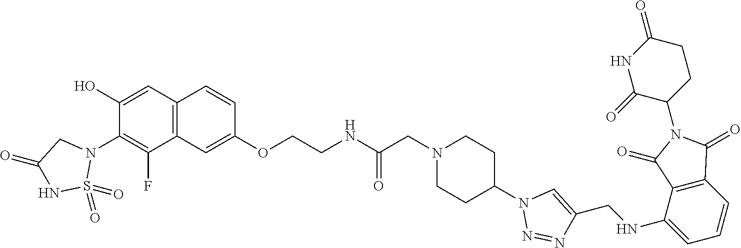 [SMILES: O=C(CN1CCC(n2cc(CNc3cccc4c3C(=O)N(C3CCC(=O)NC3=O)C4=O)nn2)CC1)NCCOc1ccc2cc(O)c(N3CC(=O)NS3(=O)=O)c(F)c2c1] |
| 41 | 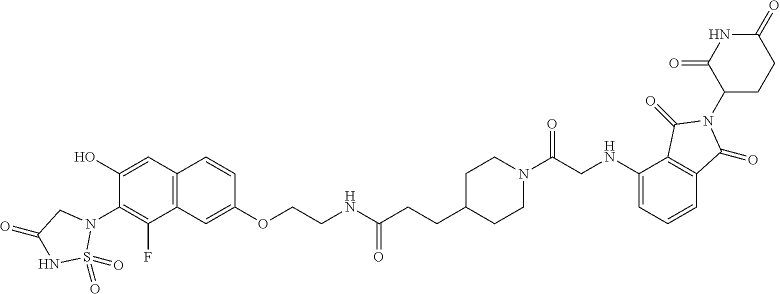 [SMILES: O=C(CCC1CCN(C(=O)CNc2cccc3c2C(=O)N(C2CCC(=O)NC2=O)C3=O)CC1)NCCOc1ccc2cc(O)c(N3CC(=O)NS3(=O)=O)c(F)c2c1] |
| 42 | 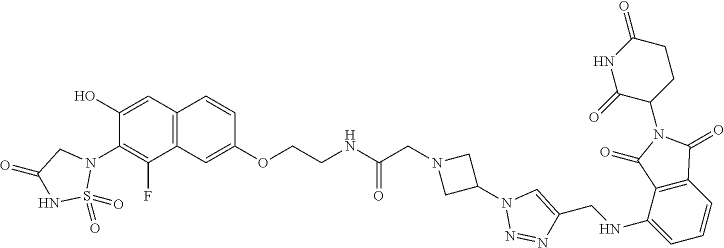 [SMILES: O=C(CN1CC(n2cc(CNc3cccc4c3C(=O)N(C3CCC(=O)NC3=O)C4=O)nn2)C1)NCCOc1ccc2cc(O)c(N3CC(=O)NS3(=O)=O)c(F)c2c1] |
| 43 | 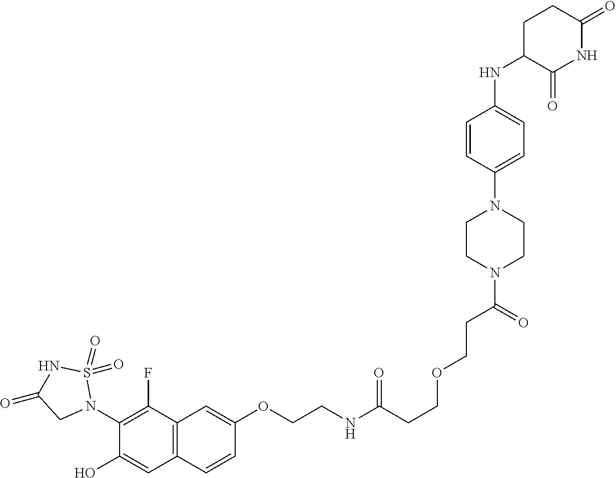 [SMILES: O=C(CCOCCC(=O)N1CCN(c2ccc(NC3CCC(=O)NC3=O)cc2)CC1)NCCOc1ccc2cc(O)c(N3CC(=O)NS3(=O)=O)c(F)c2c1] |
| 44 | 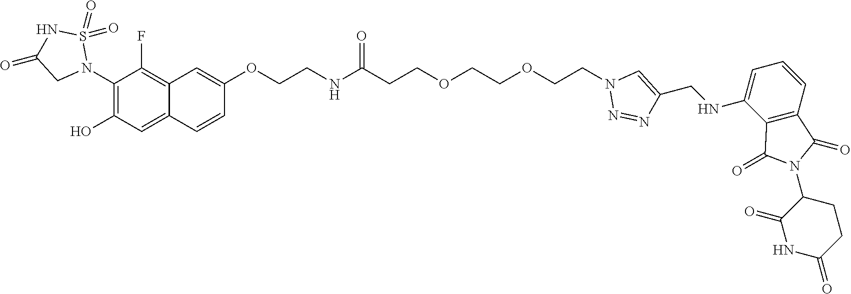 [SMILES: O=C(CCOCCOCCn1cc(CNc2cccc3c2C(=O)N(C2CCC(=O)NC2=O)C3=O)nn1)NCCOc1ccc2cc(O)c(N3CC(=O)NS3(=O)=O)c(F)c2c1] |
| 45 | 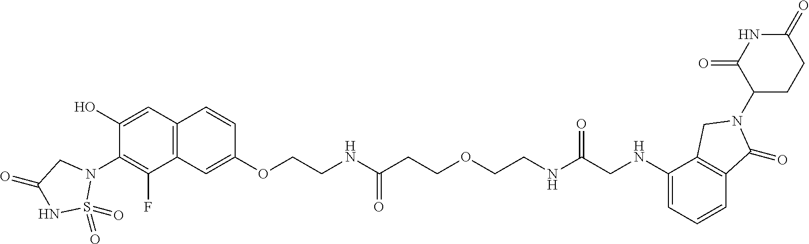 [SMILES: O=C(CCOCCNC(=O)CNc1cccc2c1CN(C1CCC(=O)NC1=O)C2=O)NCCOc1ccc2cc(O)c(N3CC(=O)NS3(=O)=O)c(F)c2c1] |
| 46 | 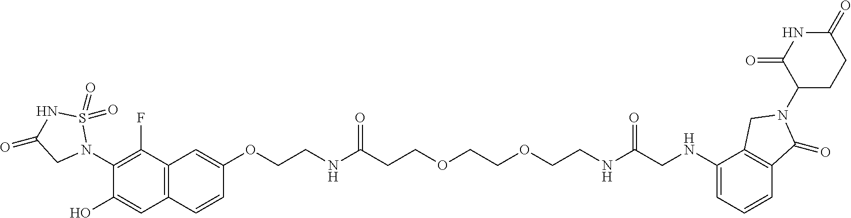 [SMILES: O=C(CCOCCOCCNC(=O)CNc1cccc2c1CN(C1CCC(=O)NC1=O)C2=O)NCCOc1ccc2cc(O)c(N3CC(=O)NS3(=O)=O)c(F)c2c1] |
| 47 | 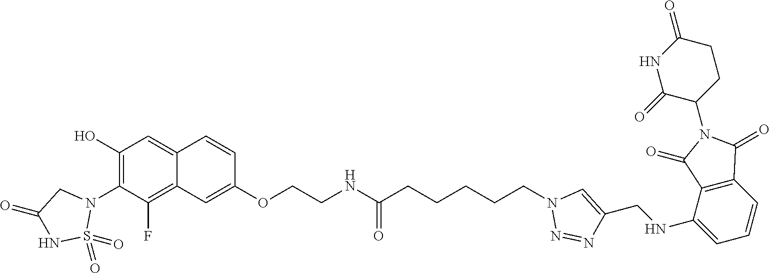 [SMILES: O=C(CCCCCn1cc(CNc2cccc3c2C(=O)N(C2CCC(=O)NC2=O)C3=O)nn1)NCCOc1ccc2cc(O)c(N3CC(=O)NS3(=O)=O)c(F)c2c1] |
| 48 | 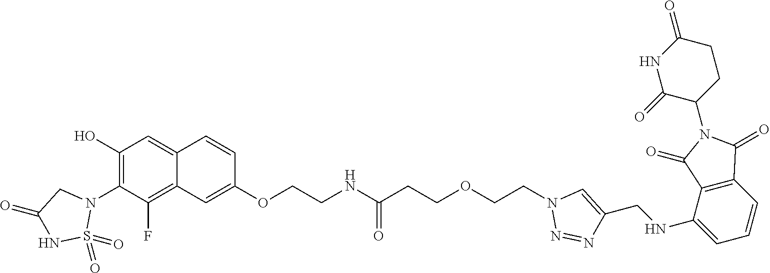 [SMILES: O=C(CCOCCn1cc(CNc2cccc3c2C(=O)N(C2CCC(=O)NC2=O)C3=O)nn1)NCCOc1ccc2cc(O)c(N3CC(=O)NS3(=O)=O)c(F)c2c1] |
| 49 | 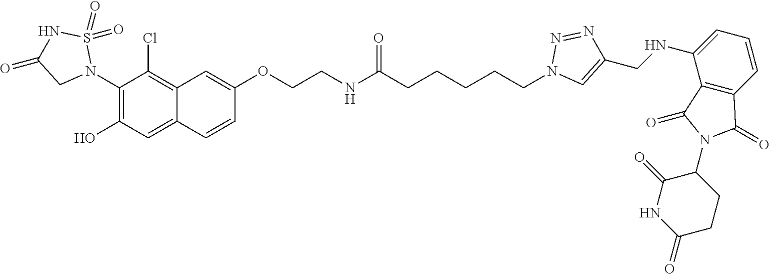 [SMILES: O=C(CCCCCn1cc(CNc2cccc3c2C(=O)N(C2CCC(=O)NC2=O)C3=O)nn1)NCCOc1ccc2cc(O)c(N3CC(=O)NS3(=O)=O)c(Cl)c2c1] |
| 50 | 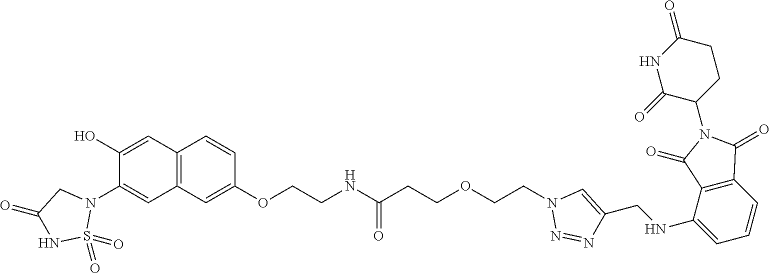 [SMILES: O=C(CCOCCn1cc(CNc2cccc3c2C(=O)N(C2CCC(=O)NC2=O)C3=O)nn1)NCCOc1ccc2cc(O)c(N3CC(=O)NS3(=O)=O)cc2c1] |
| 51 | 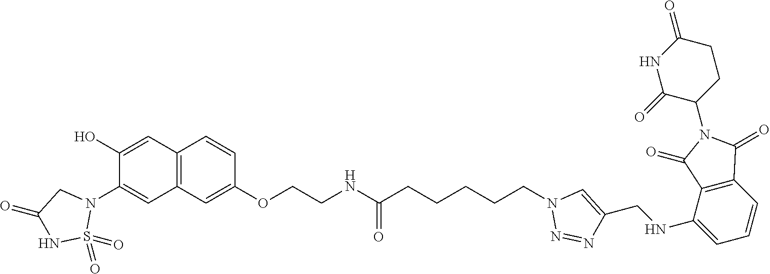 [SMILES: O=C(CCCCCn1cc(CNc2cccc3c2C(=O)N(C2CCC(=O)NC2=O)C3=O)nn1)NCCOc1ccc2cc(O)c(N3CC(=O)NS3(=O)=O)cc2c1] |
| 52 | 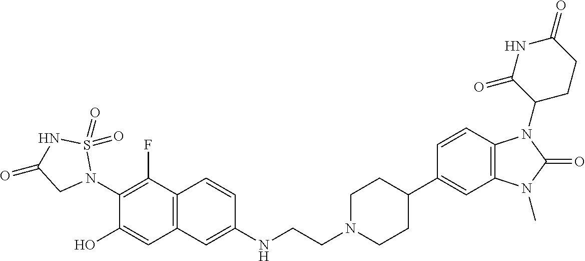 [SMILES: Cn1c(=O)n(C2CCC(=O)NC2=O)c2ccc(C3CCN(CCNc4ccc5c(F)c(N6CC(=O)NS6(=O)=O)c(O)cc5c4)CC3)cc21] |
| 53 | 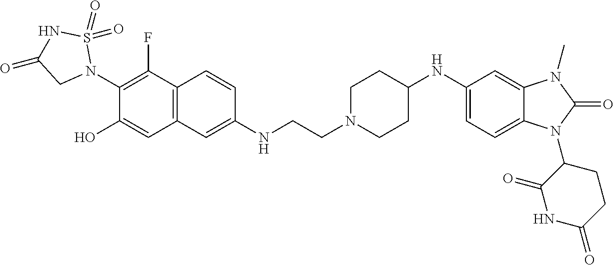 [SMILES: Cn1c(=O)n(C2CCC(=O)NC2=O)c2ccc(NC3CCN(CCNc4ccc5c(F)c(N6CC(=O)NS6(=O)=O)c(O)cc5c4)CC3)cc21] |
| 54 | 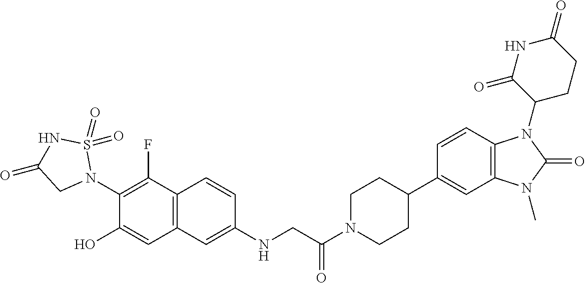 [SMILES: Cn1c(=O)n(C2CCC(=O)NC2=O)c2ccc(C3CCN(C(=O)CNc4ccc5c(F)c(N6CC(=O)NS6(=O)=O)c(O)cc5c4)CC3)cc21] |
| 55 | 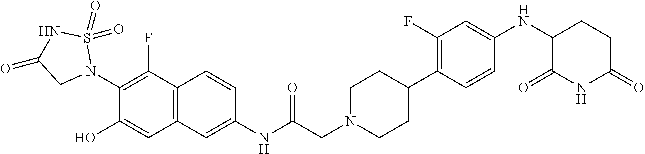 [SMILES: O=C1CCC(Nc2ccc(C3CCN(CC(=O)Nc4ccc5c(F)c(N6CC(=O)NS6(=O)=O)c(O)cc5c4)CC3)c(F)c2)C(=O)N1] |
| 56 | 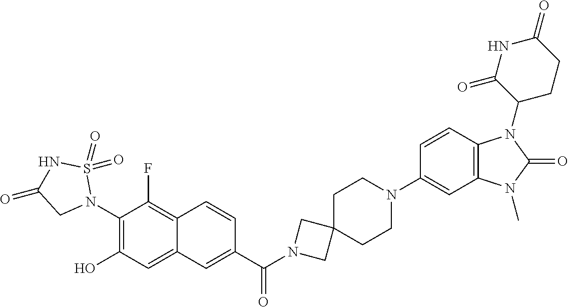 [SMILES: Cn1c(=O)n(C2CCC(=O)NC2=O)c2ccc(N3CCC4(CC3)CN(C(=O)c3ccc5c(F)c(N6CC(=O)NS6(=O)=O)c(O)cc5c3)C4)cc21] |
| 57 | 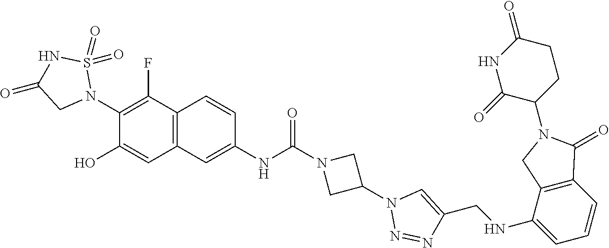 [SMILES: O=C1CCC(N2Cc3c(NCc4cn(C5CN(C(=O)Nc6ccc7c(F)c(N8CC(=O)NS8(=O)=O)c(O)cc7c6)C5)nn4)cccc3C2=O)C(=O)N1] |
| 58 | 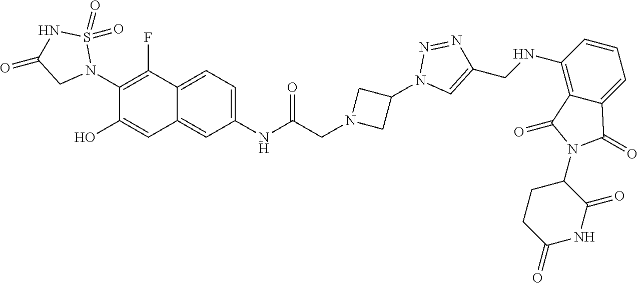 [SMILES: O=C1CCC(N2C(=O)c3cccc(NCc4cn(C5CN(CC(=O)Nc6ccc7c(F)c(N8CC(=O)NS8(=O)=O)c(O)cc7c6)C5)nn4)c3C2=O)C(=O)N1] |
| 59 | 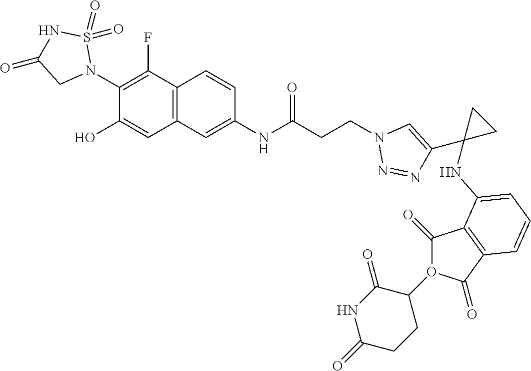 |
| 60 | 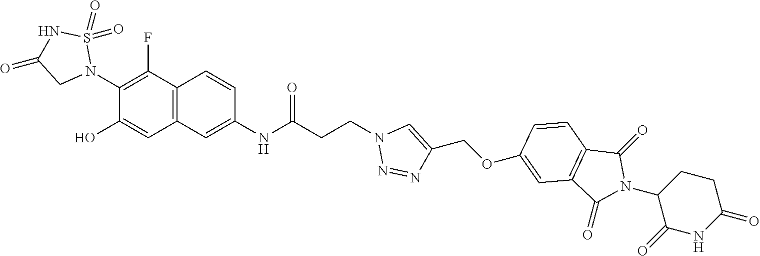 [SMILES: O=C1CCC(N2C(=O)c3ccc(OCc4cn(CCC(=O)Nc5ccc6c(F)c(N7CC(=O)NS7(=O)=O)c(O)cc6c5)nn4)cc3C2=O)C(=O)N1] |
| 61 | 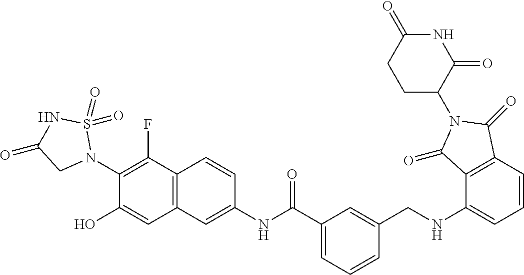 [SMILES: O=C1CCC(N2C(=O)c3cccc(NCc4cccc(C(=O)Nc5ccc6c(F)c(N7CC(=O)NS7(=O)=O)c(O)cc6c5)c4)c3C2=O)C(=O)N1] |
| 62 | 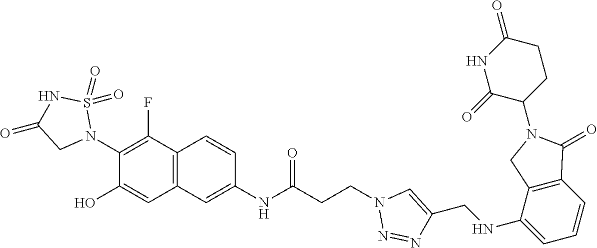 [SMILES: O=C1CCC(N2Cc3c(NCc4cn(CCC(=O)Nc5ccc6c(F)c(N7CC(=O)NS7(=O)=O)c(O)cc6c5)nn4)cccc3C2=O)C(=O)N1] |
| 63 | 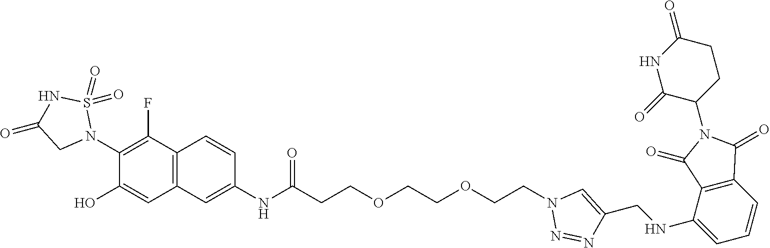 [SMILES: O=C1CCC(N2C(=O)c3cccc(NCc4cn(CCOCCOCCC(=O)Nc5ccc6c(F)c(N7CC(=O)NS7(=O)=O)c(O)cc6c5)nn4)c3C2=O)C(=O)N1] |
| 64 | 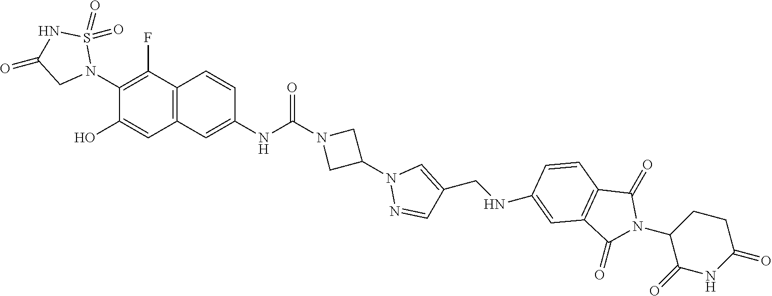 [SMILES: O=C1CCC(N2C(=O)c3ccc(NCc4cnn(C5CN(C(=O)Nc6ccc7c(F)c(N8CC(=O)NS8(=O)=O)c(O)cc7c6)C5)c4)cc3C2=O)C(=O)N1] |
| 65 | 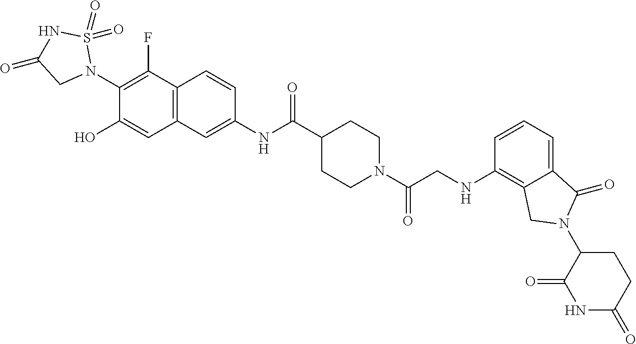 [SMILES: O=C1CCC(N2Cc3c(NCC(=O)N4CCC(C(=O)Nc5ccc6c(F)c(N7CC(=O)NS7(=O)=O)c(O)cc6c5)CC4)cccc3C2=O)C(=O)N1] |
| 66 | 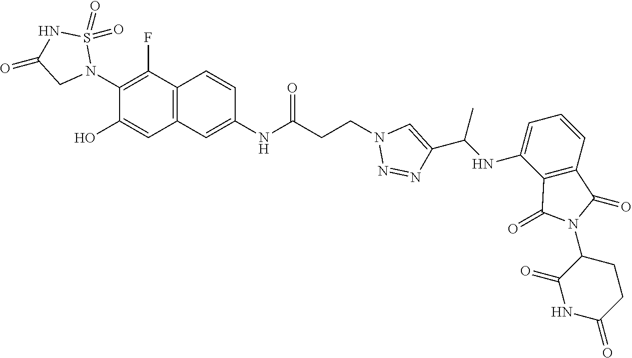 [SMILES: CC(Nc1cccc2c1C(=O)N(C1CCC(=O)NC1=O)C2=O)c1cn(CCC(=O)Nc2ccc3c(F)c(N4CC(=O)NS4(=O)=O)c(O)cc3c2)nn1] |
| 67 | 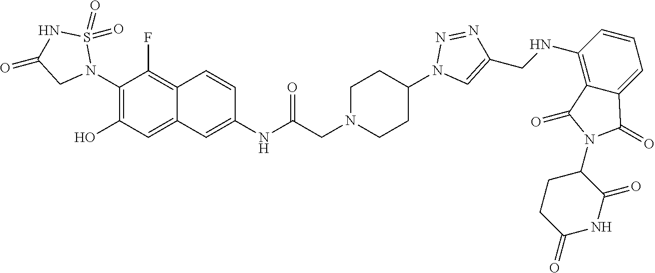 [SMILES: O=C1CCC(N2C(=O)c3cccc(NCc4cn(C5CCN(CC(=O)Nc6ccc7c(F)c(N8CC(=O)NS8(=O)=O)c(O)cc7c6)CC5)nn4)c3C2=O)C(=O)N1] |
| 68 | 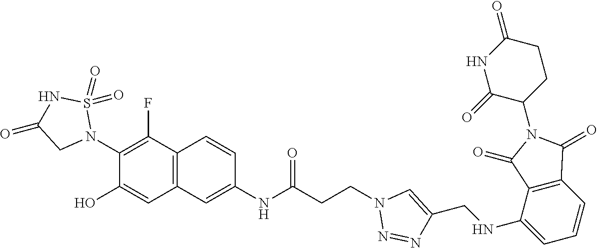 [SMILES: O=C1CCC(N2C(=O)c3cccc(NCc4cn(CCC(=O)Nc5ccc6c(F)c(N7CC(=O)NS7(=O)=O)c(O)cc6c5)nn4)c3C2=O)C(=O)N1] |
| 69 | 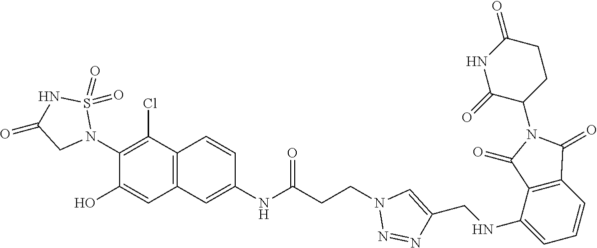 [SMILES: O=C1CCC(N2C(=O)c3cccc(NCc4cn(CCC(=O)Nc5ccc6c(Cl)c(N7CC(=O)NS7(=O)=O)c(O)cc6c5)nn4)c3C2=O)C(=O)N1] |
| 70 | 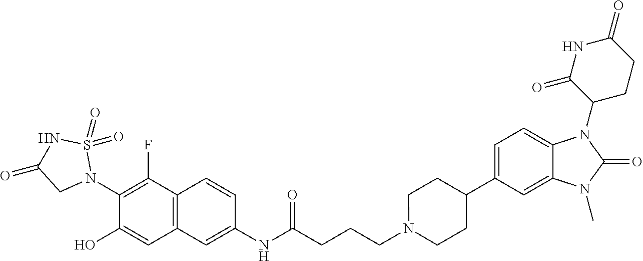 [SMILES: Cn1c(=O)n(C2CCC(=O)NC2=O)c2ccc(C3CCN(CCCC(=O)Nc4ccc5c(F)c(N6CC(=O)NS6(=O)=O)c(O)cc5c4)CC3)cc21] |
| 71 | 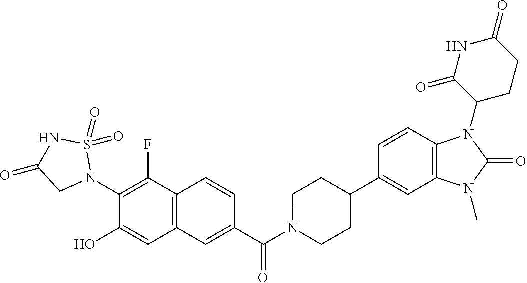 [SMILES: Cn1c(=O)n(C2CCC(=O)NC2=O)c2ccc(C3CCN(C(=O)c4ccc5c(F)c(N6CC(=O)NS6(=O)=O)c(O)cc5c4)CC3)cc21] |
| 72 | 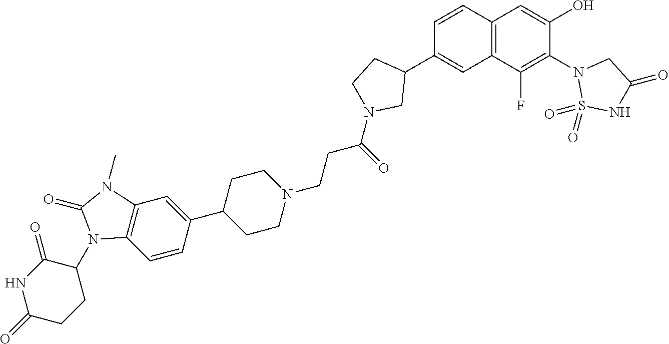 [SMILES: Cn1c(=O)n(C2CCC(=O)NC2=O)c2ccc(C3CCN(CCC(=O)N4CCC(c5ccc6cc(O)c(N7CC(=O)NS7(=O)=O)c(F)c6c5)C4)CC3)cc21] |
| 73 | 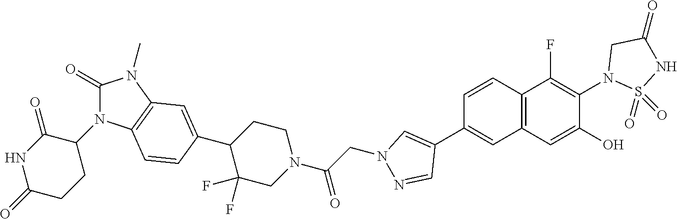 [SMILES: Cn1c(=O)n(C2CCC(=O)NC2=O)c2ccc(C3CCN(C(=O)Cn4cc(-c5ccc6c(F)c(N7CC(=O)NS7(=O)=O)c(O)cc6c5)cn4)CC3(F)F)cc21] |
| 74 | 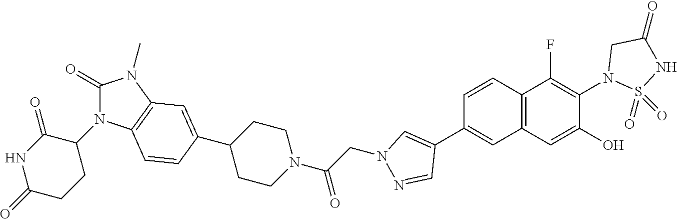 [SMILES: Cn1c(=O)n(C2CCC(=O)NC2=O)c2ccc(C3CCN(C(=O)Cn4cc(-c5ccc6c(F)c(N7CC(=O)NS7(=O)=O)c(O)cc6c5)cn4)CC3)cc21] |
| 75 | 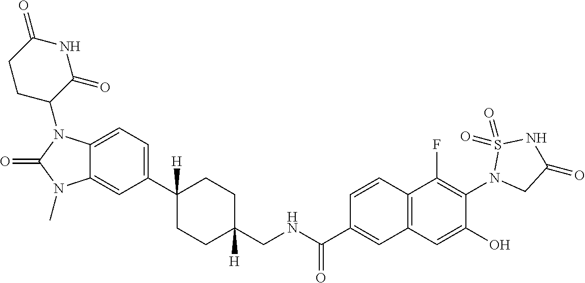 [SMILES: Cn1c(=O)n(C2CCC(=O)NC2=O)c2ccc([C@H]3CC[C@@H](CNC(=O)c4ccc5c(F)c(N6CC(=O)NS6(=O)=O)c(O)cc5c4)CC3)cc21] |
| 76 | 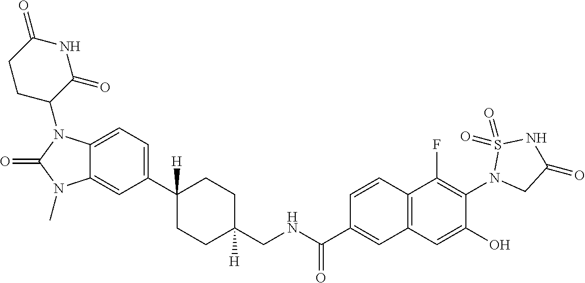 [SMILES: Cn1c(=O)n(C2CCC(=O)NC2=O)c2ccc([C@H]3CC[C@H](CNC(=O)c4ccc5c(F)c(N6CC(=O)NS6(=O)=O)c(O)cc5c4)CC3)cc21] |
| 77 | 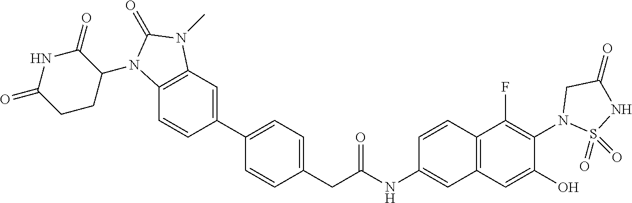 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3)cc21] |
| 78 | 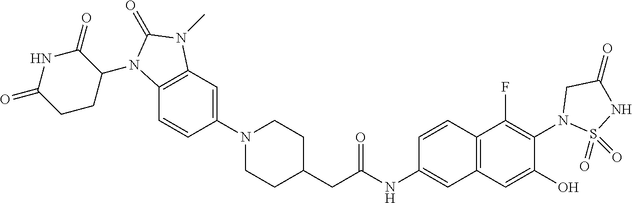 [SMILES: Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21] |
| 79 | 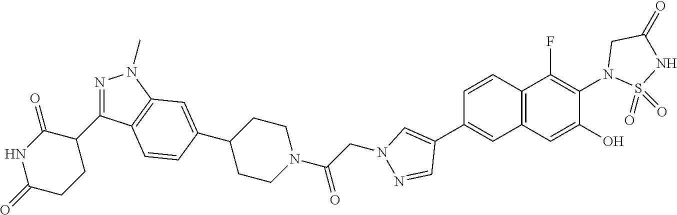 [SMILES: Cn1nc(C2CCC(=O)NC2=O)c2ccc(C3CCN(C(=O)Cn4cc(-c5ccc6c(F)c(N7CC(=O)NS7(=O)=O)c(O)cc6c5)cn4)CC3)cc21] |
| 80 | 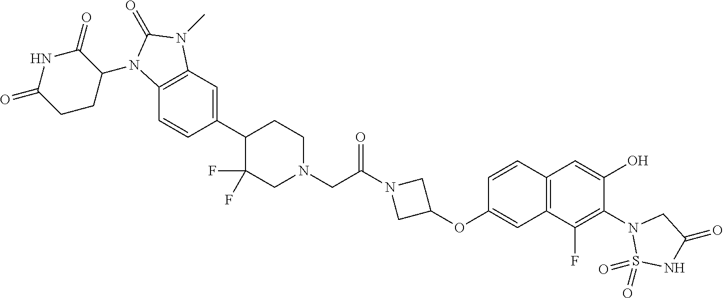 [SMILES: Cn1c(=O)n(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21] |
| 81a | 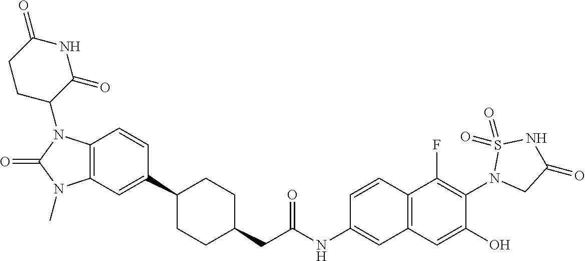 [SMILES: Cn1c(=O)n(C2CCC(=O)NC2=O)c2ccc([C@H]3CC[C@@H](CC(=O)Nc4ccc5c(F)c(N6CC(=O)NS6(=O)=O)c(O)cc5c4)CC3)cc21] |
| 81b | 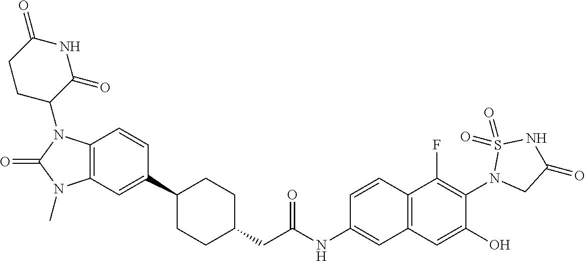 [SMILES: Cn1c(=O)n(C2CCC(=O)NC2=O)c2ccc([C@H]3CC[C@H](CC(=O)Nc4ccc5c(F)c(N6CC(=O)NS6(=O)=O)c(O)cc5c4)CC3)cc21] |
| 82 | 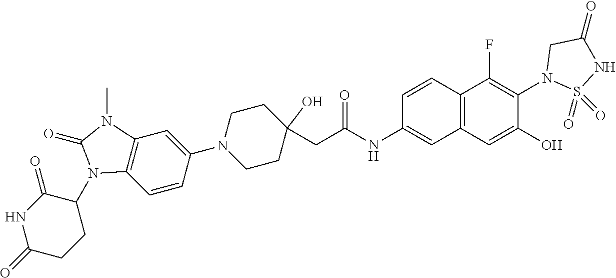 [SMILES: Cn1c(=O)n(C2CCC(=O)NC2=O)c2ccc(N3CCC(O)(CC(=O)Nc4ccc5c(F)c(N6CC(=O)NS6(=O)=O)c(O)cc5c4)CC3)cc21] |
| 83 | 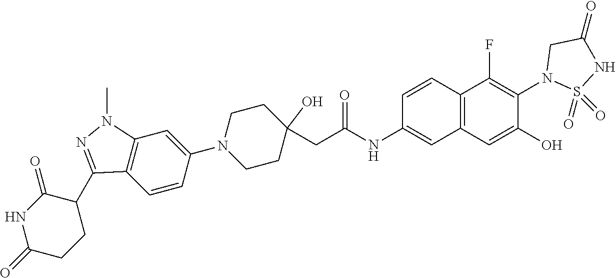 [SMILES: Cn1nc(C2CCC(=O)NC2=O)c2ccc(N3CCC(O)(CC(=O)Nc4ccc5c(F)c(N6CC(=O)NS6(=O)=O)c(O)cc5c4)CC3)cc21] |
| 84 | 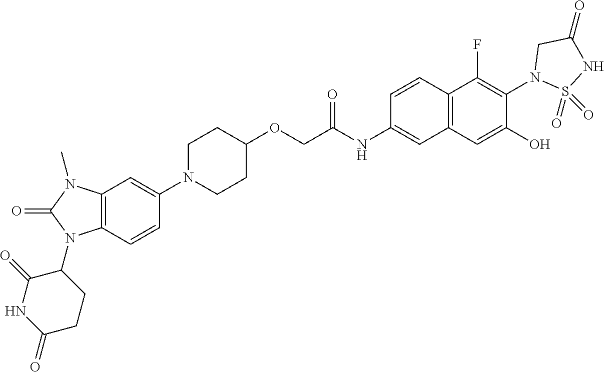 [SMILES: Cn1c(=O)n(C2CCC(=O)NC2=O)c2ccc(N3CCC(OCC(=O)Nc4ccc5c(F)c(N6CC(=O)NS6(=O)=O)c(O)cc5c4)CC3)cc21] |
| 85 | 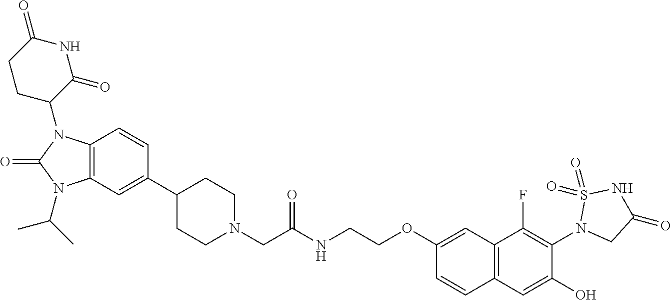 [SMILES: CC(C)n1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21] |
| 86 | 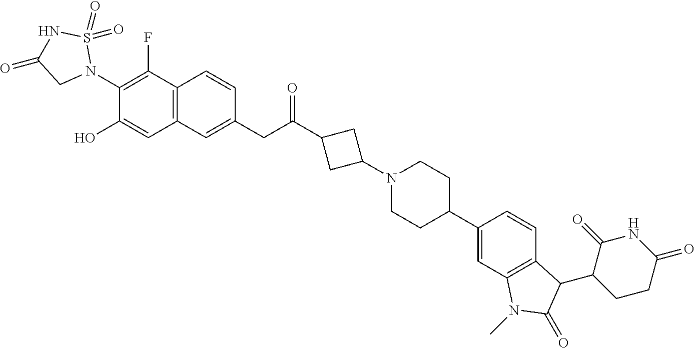 [SMILES: CN1C(=O)C(C2CCC(=O)NC2=O)c2ccc(C3CCN(C4CC(C(=O)Cc5ccc6c(F)c(N7CC(=O)NS7(=O)=O)c(O)cc6c5)C4)CC3)cc21] |
| 87 | 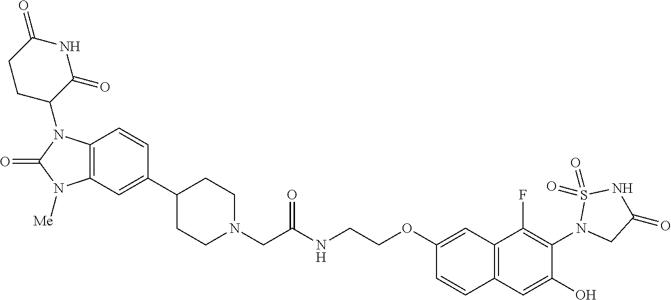 [SMILES: Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21] |
| 88 | 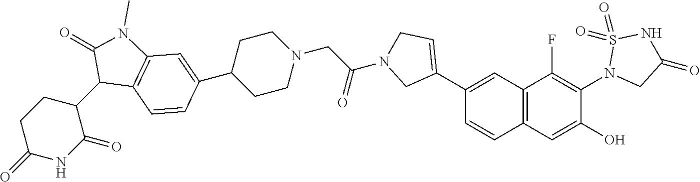 [SMILES: CN1C(=O)C(C2CCC(=O)NC2=O)c2ccc(C3CCN(CC(=O)N4CC=C(c5ccc6cc(O)c(N7CC(=O)NS7(=O)=O)c(F)c6c5)C4)CC3)cc21] |
| 89 | 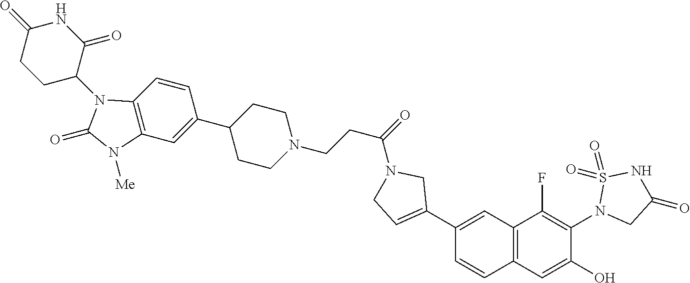 [SMILES: Cn1c(=O)n(C2CCC(=O)NC2=O)c2ccc(C3CCN(CCC(=O)N4CC=C(c5ccc6cc(O)c(N7CC(=O)NS7(=O)=O)c(F)c6c5)C4)CC3)cc21] |
| 90 | 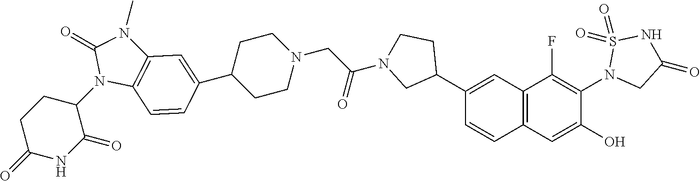 [SMILES: Cn1c(=O)n(C2CCC(=O)NC2=O)c2ccc(C3CCN(CC(=O)N4CCC(c5ccc6cc(O)c(N7CC(=O)NS7(=O)=O)c(F)c6c5)C4)CC3)cc21] |
| 91 | 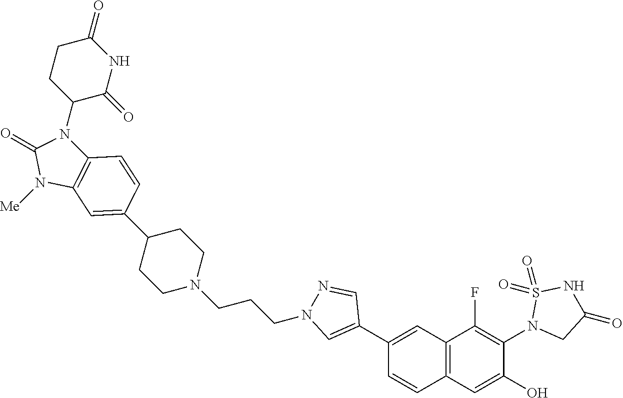 [SMILES: Cn1c(=O)n(C2CCC(=O)NC2=O)c2ccc(C3CCN(CCCn4cc(-c5ccc6cc(O)c(N7CC(=O)NS7(=O)=O)c(F)c6c5)cn4)CC3)cc21] |
| 92 | 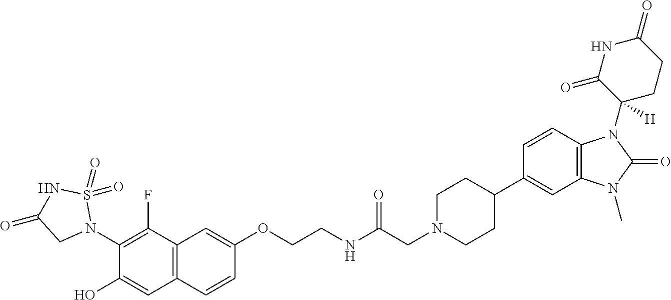 [SMILES: Cn1c(=O)n([C@@H]2CCC(=O)NC2=O)c2ccc(C3CCN(CC(=O)NCCOc4ccc5cc(O)c(N6CC(=O)NS6(=O)=O)c(F)c5c4)CC3)cc21] |
| 93 | 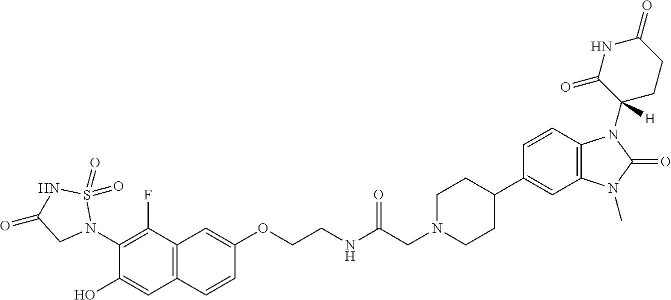 [SMILES: Cn1c(=O)n([C@H]2CCC(=O)NC2=O)c2ccc(C3CCN(CC(=O)NCCOc4ccc5cc(O)c(N6CC(=O)NS6(=O)=O)c(F)c5c4)CC3)cc21] |
| 94 | 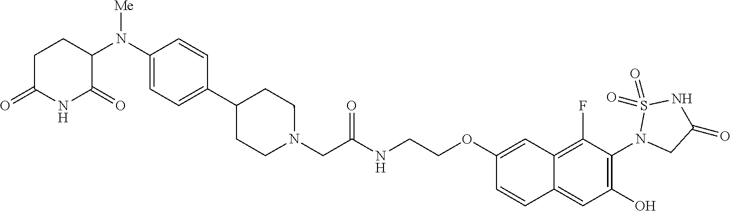 [SMILES: CN(c1ccc(C2CCN(CC(=O)NCCOc3ccc4cc(O)c(N5CC(=O)NS5(=O)=O)c(F)c4c3)CC2)cc1)C1CCC(=O)NC1=O] |
| 95 | 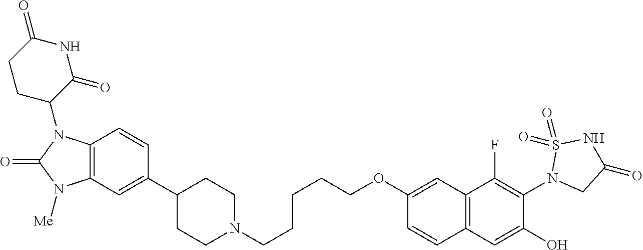 [SMILES: Cn1c(=O)n(C2CCC(=O)NC2=O)c2ccc(C3CCN(CCCCCOc4ccc5cc(O)c(N6CC(=O)NS6(=O)=O)c(F)c5c4)CC3)cc21] |
| 96 | 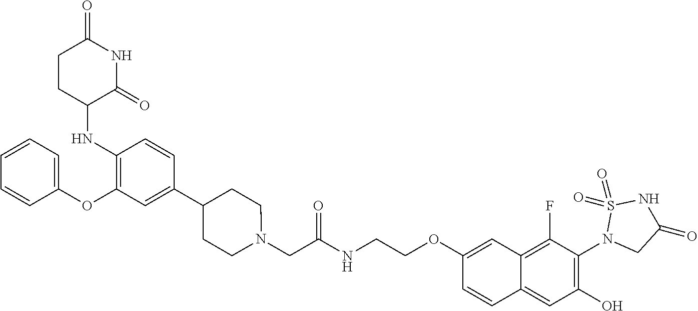 [SMILES: O=C(CN1CCC(c2ccc(NC3CCC(=O)NC3=O)c(Oc3ccccc3)c2)CC1)NCCOc1ccc2cc(O)c(N3CC(=O)NS3(=O)=O)c(F)c2c1] |
| 97 | 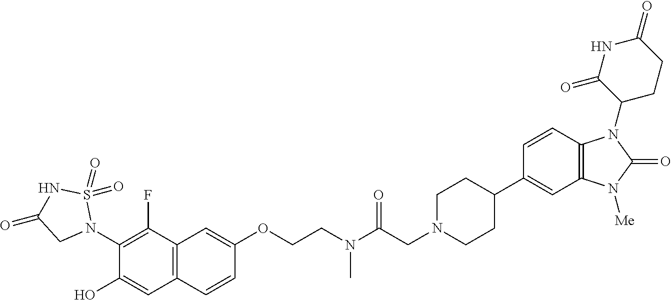 [SMILES: CN(CCOc1ccc2cc(O)c(N3CC(=O)NS3(=O)=O)c(F)c2c1)C(=O)CN1CCC(c2ccc3c(c2)n(C)c(=O)n3C2CCC(=O)NC2=O)CC1] |
| 98 | 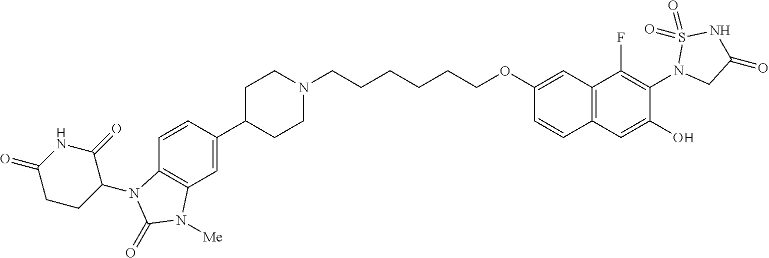 [SMILES: Cn1c(=O)n(C2CCC(=O)NC2=O)c2ccc(C3CCN(CCCCCCOc4ccc5cc(O)c(N6CC(=O)NS6(=O)=O)c(F)c5c4)CC3)cc21] |
| 99 | 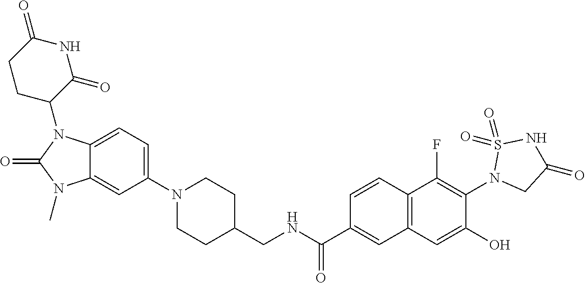 [SMILES: Cn1c(=O)n(C2CCC(=O)NC2=O)c2ccc(N3CCC(CNC(=O)c4ccc5c(F)c(N6CC(=O)NS6(=O)=O)c(O)cc5c4)CC3)cc21] |
| 100 | 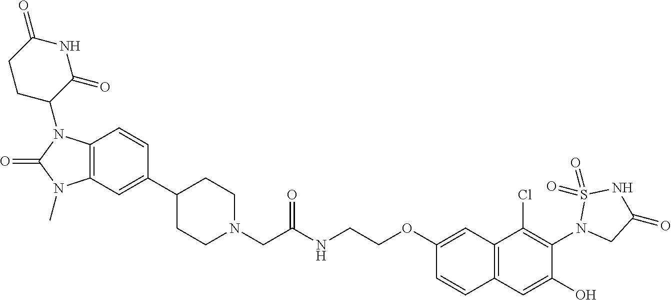 [SMILES: Cn1c(=O)n(C2CCC(=O)NC2=O)c2ccc(C3CCN(CC(=O)NCCOc4ccc5cc(O)c(N6CC(=O)NS6(=O)=O)c(Cl)c5c4)CC3)cc21] |
| 101 | 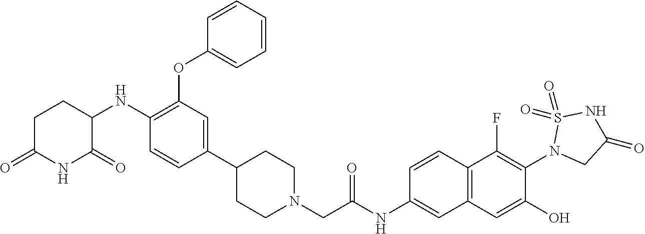 [SMILES: O=C1CCC(Nc2ccc(C3CCN(CC(=O)Nc4ccc5c(F)c(N6CC(=O)NS6(=O)=O)c(O)cc5c4)CC3)cc2Oc2ccccc2)C(=O)N1] |
| 102 | 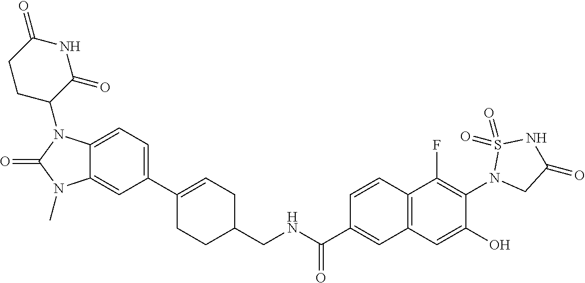 [SMILES: Cn1c(=O)n(C2CCC(=O)NC2=O)c2ccc(C3=CCC(CNC(=O)c4ccc5c(F)c(N6CC(=O)NS6(=O)=O)c(O)cc5c4)CC3)cc21] |
| 103 | 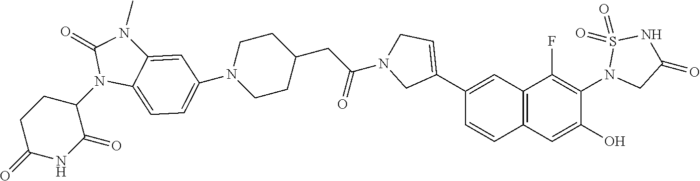 [SMILES: Cn1c(=O)n(C2CCC(=O)NC2=O)c2ccc(N3CCC(CC(=O)N4CC=C(c5ccc6cc(O)c(N7CC(=O)NS7(=O)=O)c(F)c6c5)C4)CC3)cc21] |
| 104 | 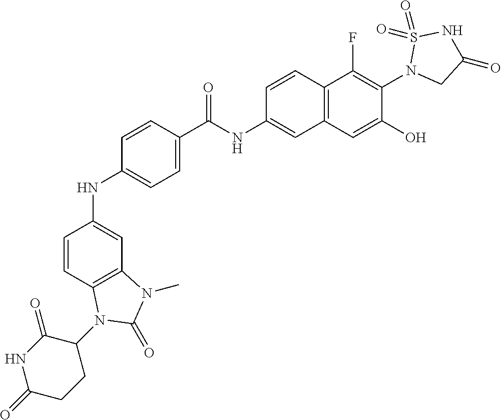 [SMILES: Cn1c(=O)n(C2CCC(=O)NC2=O)c2ccc(Nc3ccc(C(=O)Nc4ccc5c(F)c(N6CC(=O)NS6(=O)=O)c(O)cc5c4)cc3)cc21] |
| 105 | 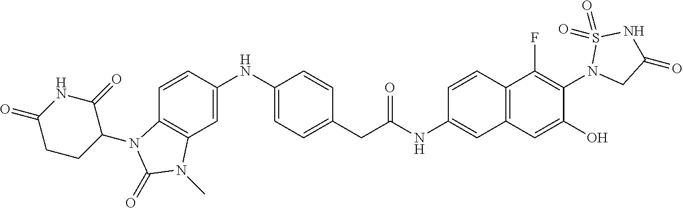 [SMILES: Cn1c(=O)n(C2CCC(=O)NC2=O)c2ccc(Nc3ccc(CC(=O)Nc4ccc5c(F)c(N6CC(=O)NS6(=O)=O)c(O)cc5c4)cc3)cc21] |
| 106 | 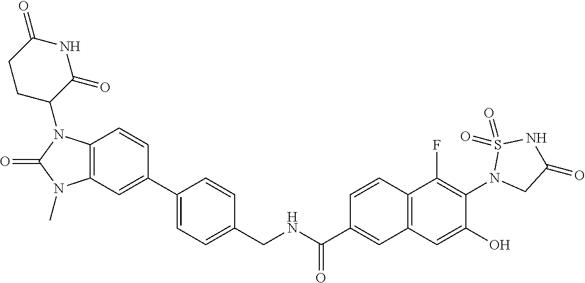 [SMILES: Cn1c(=O)n(C2CCC(=O)NC2=O)c2ccc(-c3ccc(CNC(=O)c4ccc5c(F)c(N6CC(=O)NS6(=O)=O)c(O)cc5c4)cc3)cc21] |
| 107 | 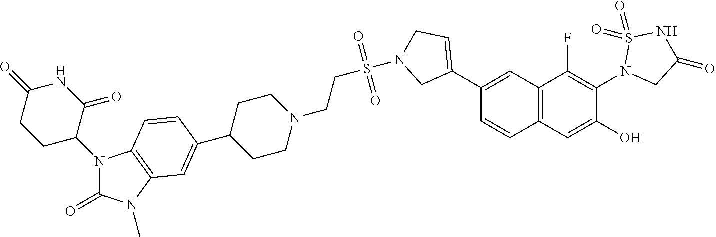 [SMILES: Cn1c(=O)n(C2CCC(=O)NC2=O)c2ccc(C3CCN(CCS(=O)(=O)N4CC=C(c5ccc6cc(O)c(N7CC(=O)NS7(=O)=O)c(F)c6c5)C4)CC3)cc21] |
| 108 | 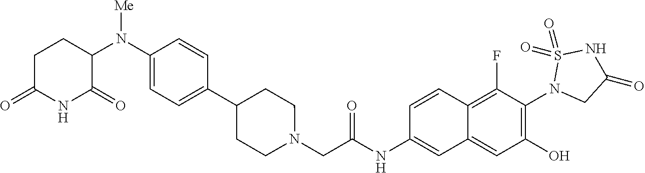 [SMILES: CN(c1ccc(C2CCN(CC(=O)Nc3ccc4c(F)c(N5CC(=O)NS5(=O)=O)c(O)cc4c3)CC2)cc1)C1CCC(=O)NC1=O] |
| 109 | 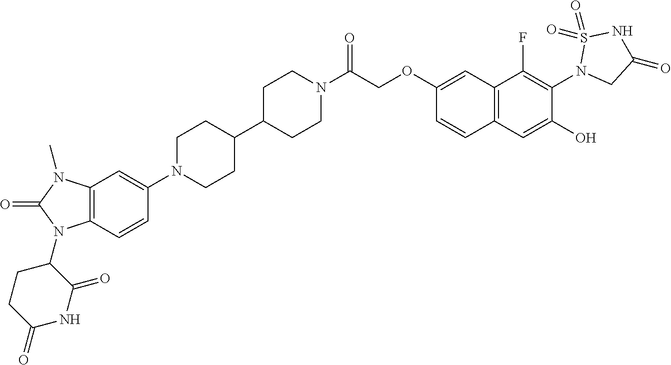 [SMILES: Cn1c(=O)n(C2CCC(=O)NC2=O)c2ccc(N3CCC(C4CCN(C(=O)COc5ccc6cc(O)c(N7CC(=O)NS7(=O)=O)c(F)c6c5)CC4)CC3)cc21] |
| 110 | 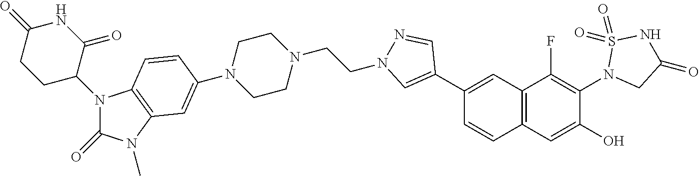 [SMILES: Cn1c(=O)n(C2CCC(=O)NC2=O)c2ccc(N3CCN(CCn4cc(-c5ccc6cc(O)c(N7CC(=O)NS7(=O)=O)c(F)c6c5)cn4)CC3)cc21] |
| 111 | 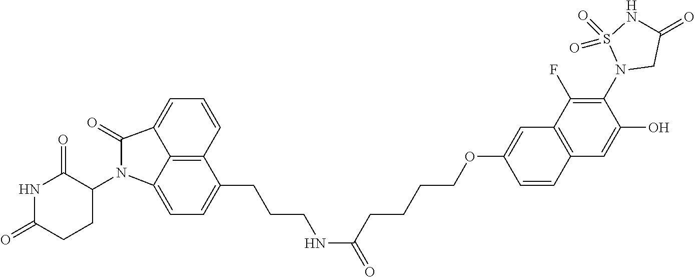 [SMILES: O=C(CCCCOc1ccc2cc(O)c(N3CC(=O)NS3(=O)=O)c(F)c2c1)NCCCc1ccc2c3c(cccc13)C(=O)N2C1CCC(=O)NC1=O] |
| 112 | 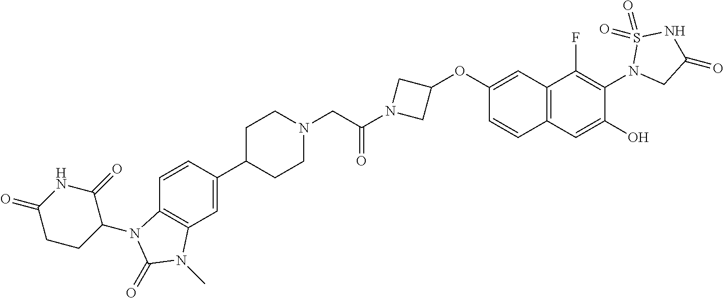 [SMILES: Cn1c(=O)n(C2CCC(=O)NC2=O)c2ccc(C3CCN(CC(=O)N4CC(Oc5ccc6cc(O)c(N7CC(=O)NS7(=O)=O)c(F)c6c5)C4)CC3)cc21] |
| 113 | 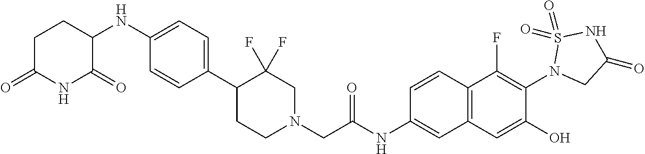 [SMILES: O=C1CCC(Nc2ccc(C3CCN(CC(=O)Nc4ccc5c(F)c(N6CC(=O)NS6(=O)=O)c(O)cc5c4)CC3(F)F)cc2)C(=O)N1] |
| 114 | 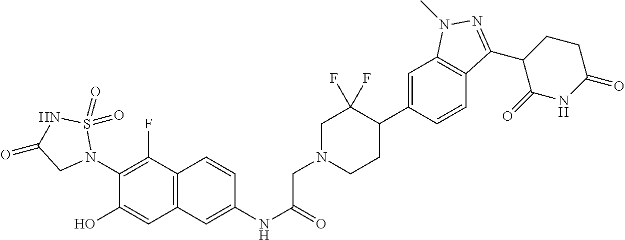 [SMILES: Cn1nc(C2CCC(=O)NC2=O)c2ccc(C3CCN(CC(=O)Nc4ccc5c(F)c(N6CC(=O)NS6(=O)=O)c(O)cc5c4)CC3(F)F)cc21] |
| 115 | 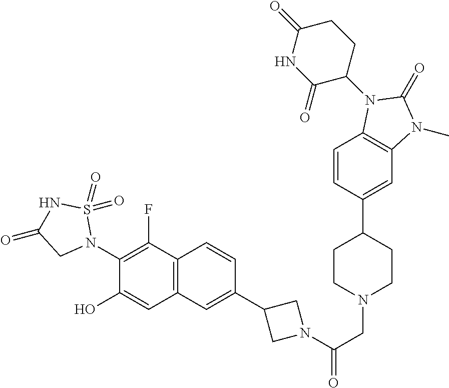 [SMILES: Cn1c(=O)n(C2CCC(=O)NC2=O)c2ccc(C3CCN(CC(=O)N4CC(c5ccc6c(F)c(N7CC(=O)NS7(=O)=O)c(O)cc6c5)C4)CC3)cc21] |
| 116 | 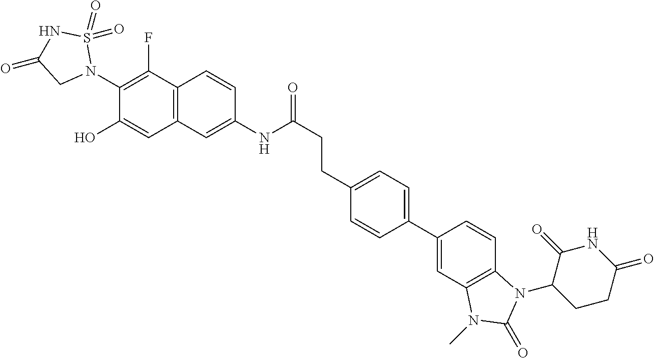 [SMILES: Cn1c(=O)n(C2CCC(=O)NC2=O)c2ccc(-c3ccc(CCC(=O)Nc4ccc5c(F)c(N6CC(=O)NS6(=O)=O)c(O)cc5c4)cc3)cc21] |
| 117 | 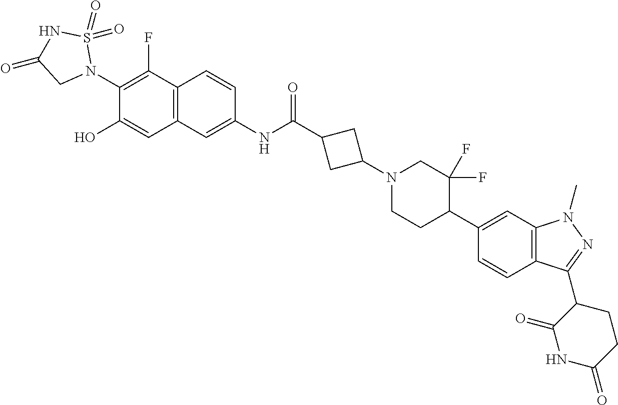 [SMILES: Cn1nc(C2CCC(=O)NC2=O)c2ccc(C3CCN(C4CC(C(=O)Nc5ccc6c(F)c(N7CC(=O)NS7(=O)=O)c(O)cc6c5)C4)CC3(F)F)cc21] |
| 118 | 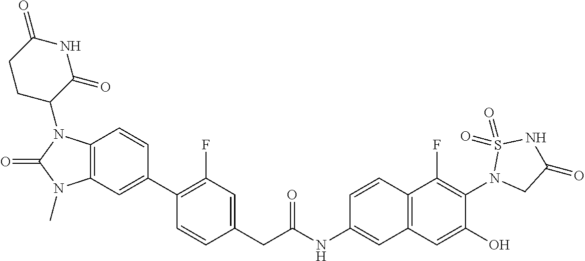 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3F)cc21] |
| 119 | 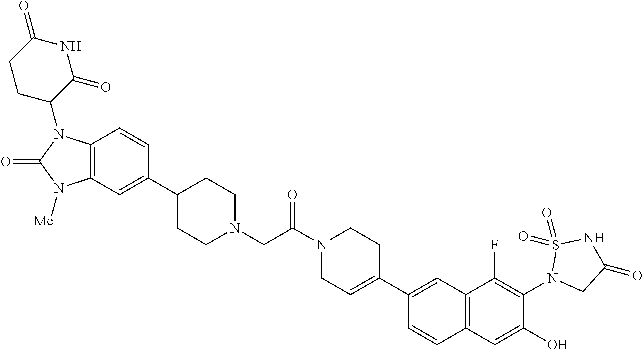 [SMILES: Cn1c(=O)n(C2CCC(=O)NC2=O)c2ccc(C3CCN(CC(=O)N4CC=C(c5ccc6cc(O)c(N7CC(=O)NS7(=O)=O)c(F)c6c5)CC4)CC3)cc21] |
| 120 | 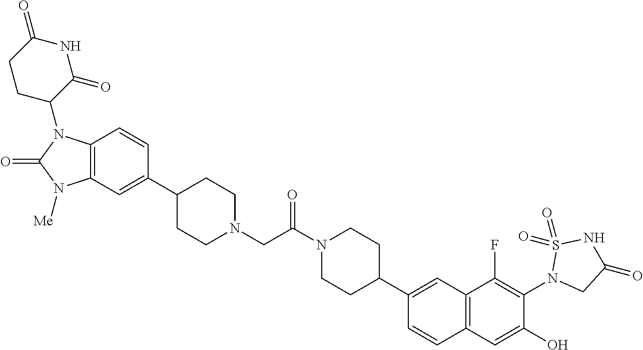 [SMILES: Cn1c(=O)n(C2CCC(=O)NC2=O)c2ccc(C3CCN(CC(=O)N4CCC(c5ccc6cc(O)c(N7CC(=O)NS7(=O)=O)c(F)c6c5)CC4)CC3)cc21] |
| 121 | 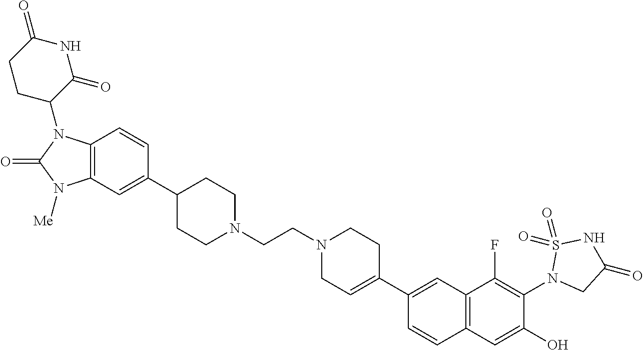 [SMILES: Cn1c(=O)n(C2CCC(=O)NC2=O)c2ccc(C3CCN(CCN4CC=C(c5ccc6cc(O)c(N7CC(=O)NS7(=O)=O)c(F)c6c5)CC4)CC3)cc21] |
| 122 | 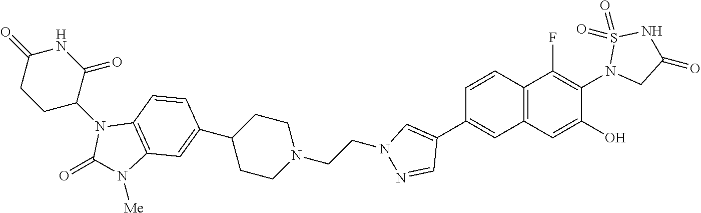 [SMILES: Cn1c(=O)n(C2CCC(=O)NC2=O)c2ccc(C3CCN(CCn4cc(-c5ccc6c(F)c(N7CC(=O)NS7(=O)=O)c(O)cc6c5)cn4)CC3)cc21] |
| 123 | 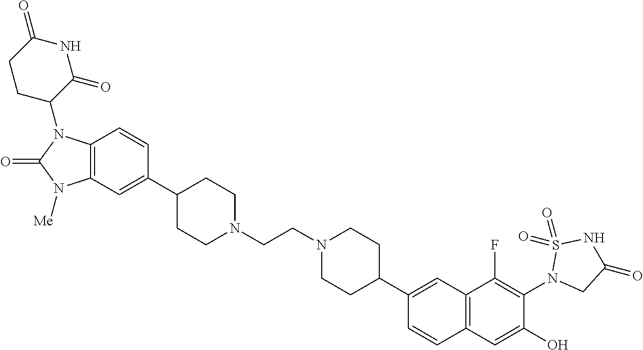 [SMILES: Cn1c(=O)n(C2CCC(=O)NC2=O)c2ccc(C3CCN(CCN4CCC(c5ccc6cc(O)c(N7CC(=O)NS7(=O)=O)c(F)c6c5)CC4)CC3)cc21] |
| 124 | 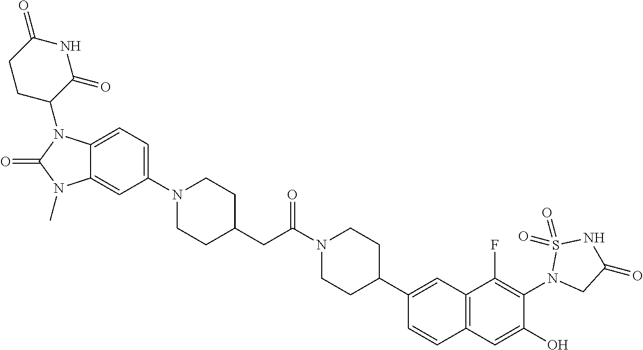 [SMILES: Cn1c(=O)n(C2CCC(=O)NC2=O)c2ccc(N3CCC(CC(=O)N4CCC(c5ccc6cc(O)c(N7CC(=O)NS7(=O)=O)c(F)c6c5)CC4)CC3)cc21] |
| 125 | 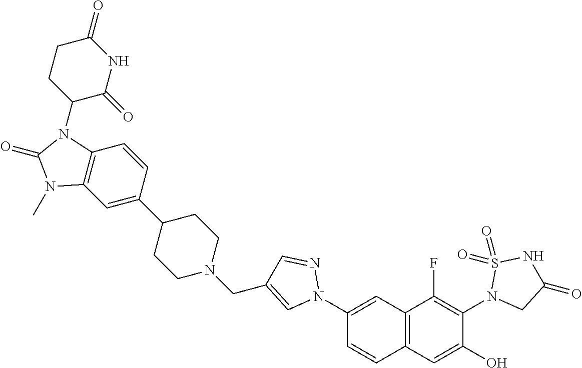 [SMILES: Cn1c(=O)n(C2CCC(=O)NC2=O)c2ccc(C3CCN(Cc4cnn(-c5ccc6cc(O)c(N7CC(=O)NS7(=O)=O)c(F)c6c5)c4)CC3)cc21] |
| 126 | 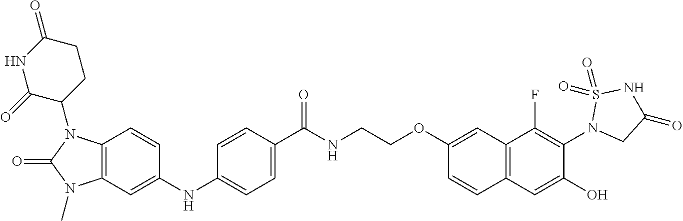 [SMILES: Cn1c(=O)n(C2CCC(=O)NC2=O)c2ccc(Nc3ccc(C(=O)NCCOc4ccc5cc(O)c(N6CC(=O)NS6(=O)=O)c(F)c5c4)cc3)cc21] |
| 127 | 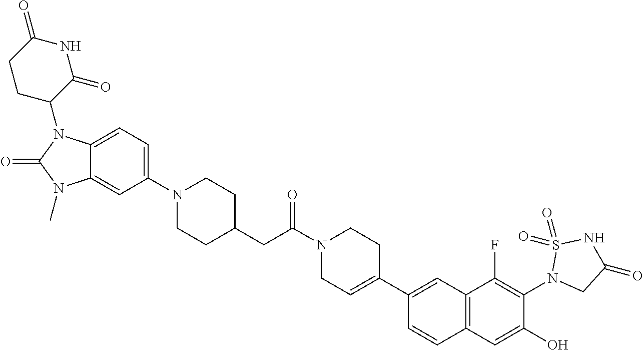 [SMILES: Cn1c(=O)n(C2CCC(=O)NC2=O)c2ccc(N3CCC(CC(=O)N4CC=C(c5ccc6cc(O)c(N7CC(=O)NS7(=O)=O)c(F)c6c5)CC4)CC3)cc21] |
| 128 | 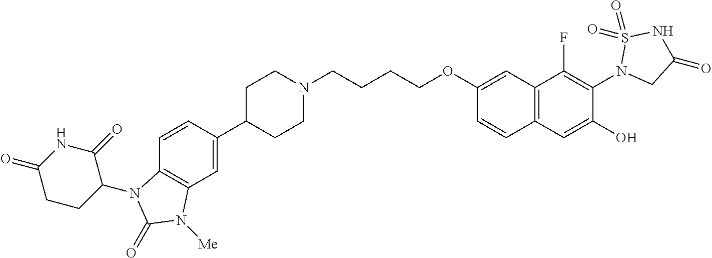 [SMILES: Cn1c(=O)n(C2CCC(=O)NC2=O)c2ccc(C3CCN(CCCCOc4ccc5cc(O)c(N6CC(=O)NS6(=O)=O)c(F)c5c4)CC3)cc21] |
| 129 | 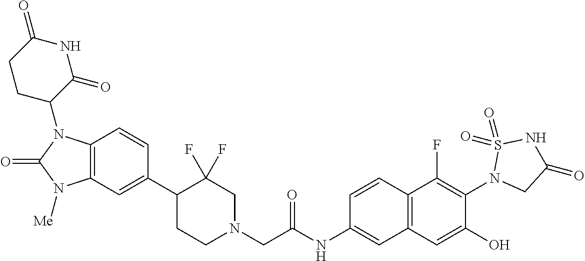 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3(F)F)cc21] |
| 130 | 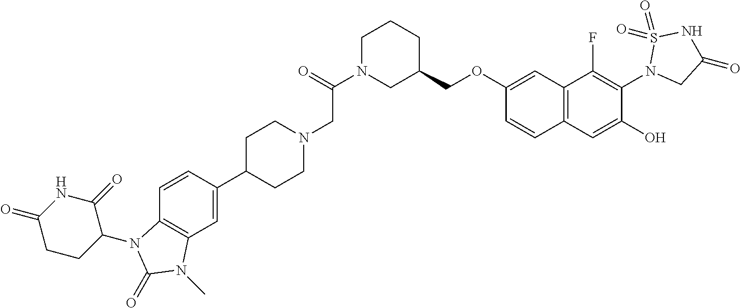 [SMILES: Cn1c(=O)n(C2CCC(=O)NC2=O)c2ccc(C3CCN(CC(=O)N4CCC[C@@H](COc5ccc6cc(O)c(N7CC(=O)NS7(=O)=O)c(F)c6c5)C4)CC3)cc21] |
| 131 | 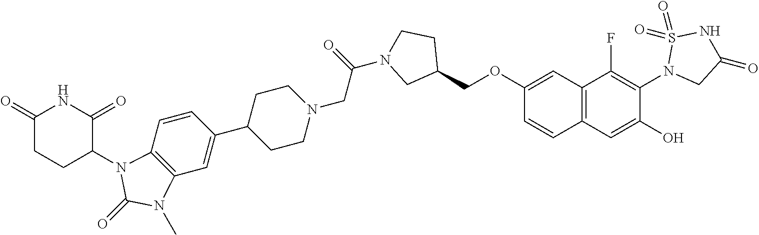 [SMILES: Cn1c(=O)n(C2CCC(=O)NC2=O)c2ccc(C3CCN(CC(=O)N4CC[C@@H](COc5ccc6cc(O)c(N7CC(=O)NS7(=O)=O)c(F)c6c5)C4)CC3)cc21] |
| 132 | 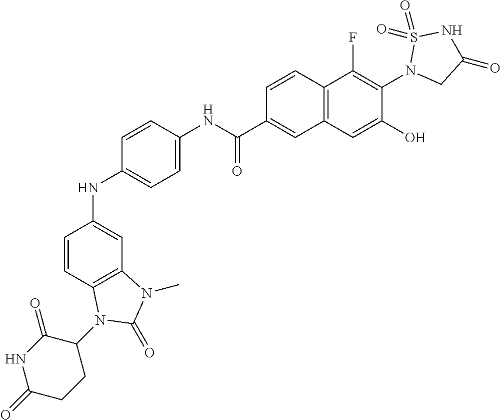 [SMILES: Cn1c(=O)n(C2CCC(=O)NC2=O)c2ccc(Nc3ccc(NC(=O)c4ccc5c(F)c(N6CC(=O)NS6(=O)=O)c(O)cc5c4)cc3)cc21] |
| 133 | 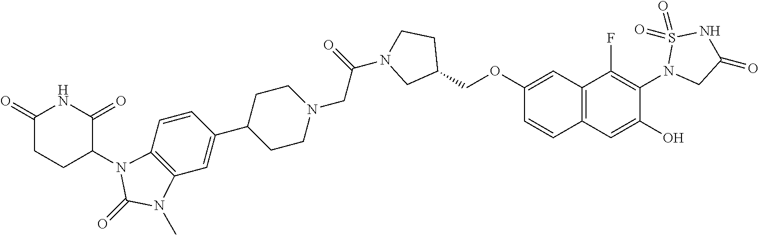 [SMILES: Cn1c(=O)n(C2CCC(=O)NC2=O)c2ccc(C3CCN(CC(=O)N4CC[C@H](COc5ccc6cc(O)c(N7CC(=O)NS7(=O)=O)c(F)c6c5)C4)CC3)cc21] |
| 134 | 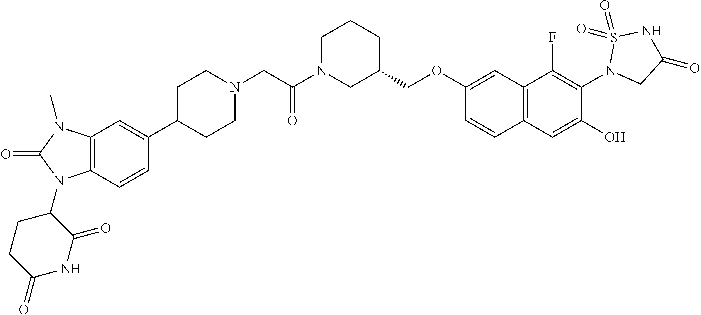 [SMILES: Cn1c(=O)n(C2CCC(=O)NC2=O)c2ccc(C3CCN(CC(=O)N4CCC[C@H](COc5ccc6cc(O)c(N7CC(=O)NS7(=O)=O)c(F)c6c5)C4)CC3)cc21] |
| 135 | 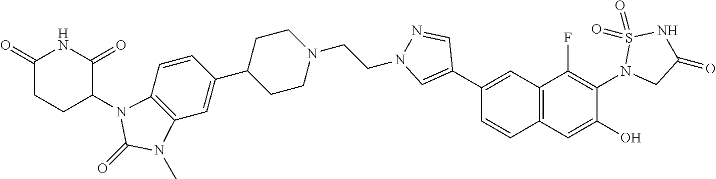 [SMILES: Cn1c(=O)n(C2CCC(=O)NC2=O)c2ccc(C3CCN(CCn4cc(-c5ccc6cc(O)c(N7CC(=O)NS7(=O)=O)c(F)c6c5)cn4)CC3)cc21] |
| 136 | 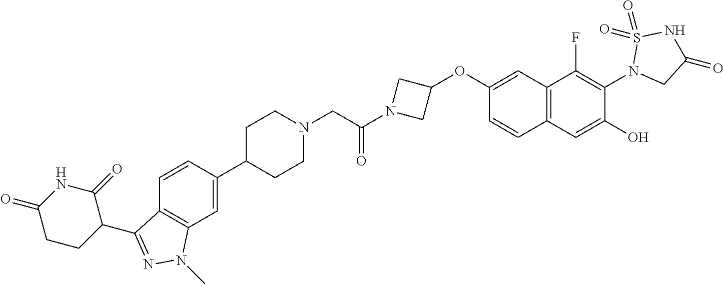 [SMILES: Cn1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3)cc21] |
| 137 | 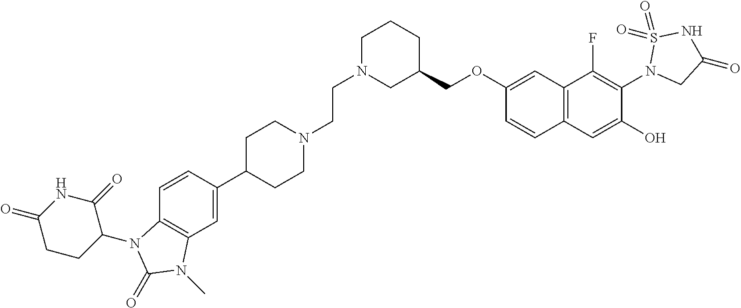 [SMILES: Cn1c(=O)n(C2CCC(=O)NC2=O)c2ccc(C3CCN(CCN4CCC[C@@H](COc5ccc6cc(O)c(N7CC(=O)NS7(=O)=O)c(F)c6c5)C4)CC3)cc21] |
| 138 | 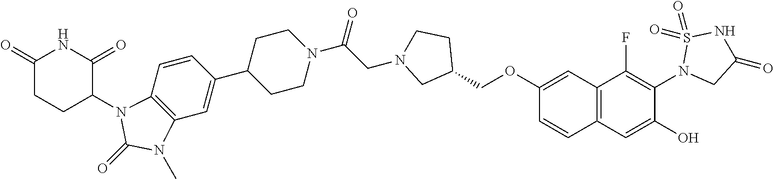 [SMILES: Cn1c(=O)n(C2CCC(=O)NC2=O)c2ccc(C3CCN(C(=O)CN4CC[C@H](COc5ccc6cc(O)c(N7CC(=O)NS7(=O)=O)c(F)c6c5)C4)CC3)cc21] |
| 139 | 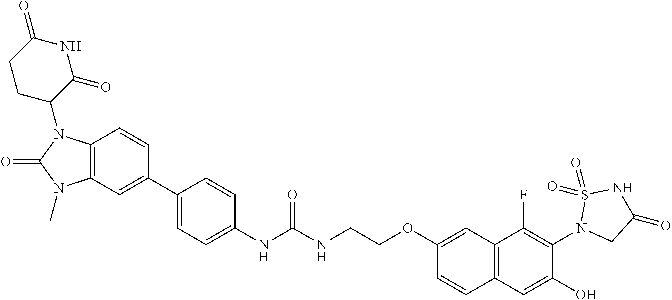 [SMILES: Cn1c(=O)n(C2CCC(=O)NC2=O)c2ccc(-c3ccc(NC(=O)NCCOc4ccc5cc(O)c(N6CC(=O)NS6(=O)=O)c(F)c5c4)cc3)cc21] |
| 140 | 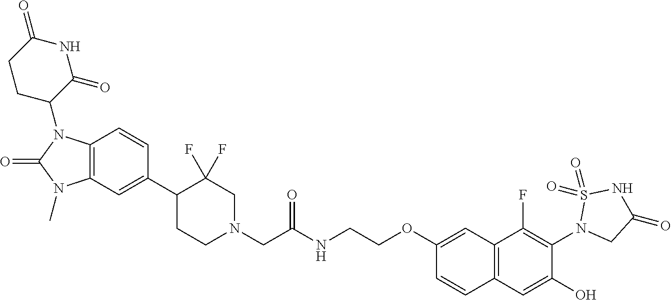 [SMILES: Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3(F)F)cc21] |
| 141 | 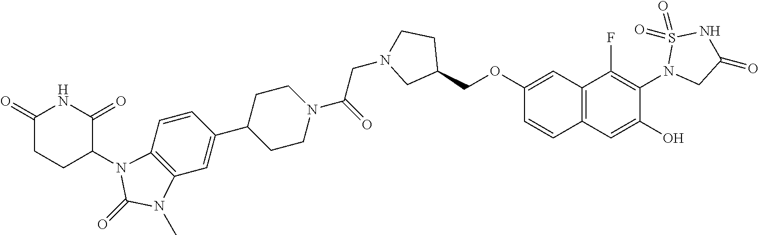 [SMILES: Cn1c(=O)n(C2CCC(=O)NC2=O)c2ccc(C3CCN(C(=O)CN4CC[C@@H](COc5ccc6cc(O)c(N7CC(=O)NS7(=O)=O)c(F)c6c5)C4)CC3)cc21] |
| 142 | 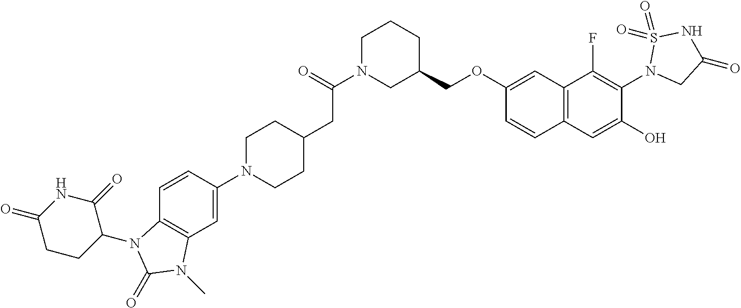 [SMILES: Cn1c(=O)n(C2CCC(=O)NC2=O)c2ccc(N3CCC(CC(=O)N4CCC[C@@H](COc5ccc6cc(O)c(N7CC(=O)NS7(=O)=O)c(F)c6c5)C4)CC3)cc21] |
| 143 | 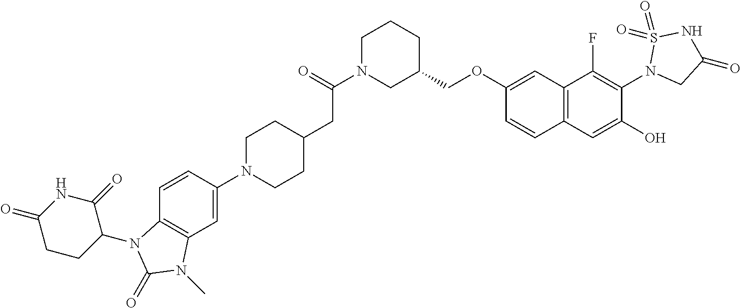 [SMILES: Cn1c(=O)n(C2CCC(=O)NC2=O)c2ccc(N3CCC(CC(=O)N4CCC[C@H](COc5ccc6cc(O)c(N7CC(=O)NS7(=O)=O)c(F)c6c5)C4)CC3)cc21] |
| 144 | 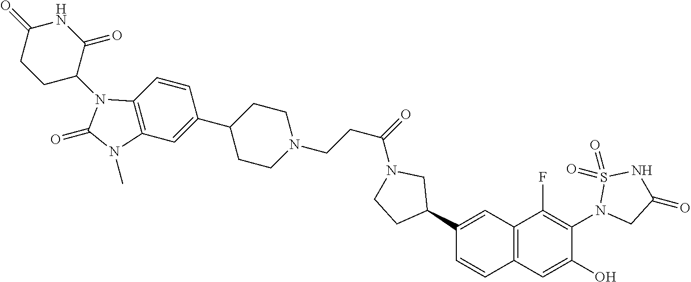 [SMILES: Cn1c(=O)n(C2CCC(=O)NC2=O)c2ccc(C3CCN(CCC(=O)N4CC[C@H](c5ccc6cc(O)c(N7CC(=O)NS7(=O)=O)c(F)c6c5)C4)CC3)cc21] |
| 145 | 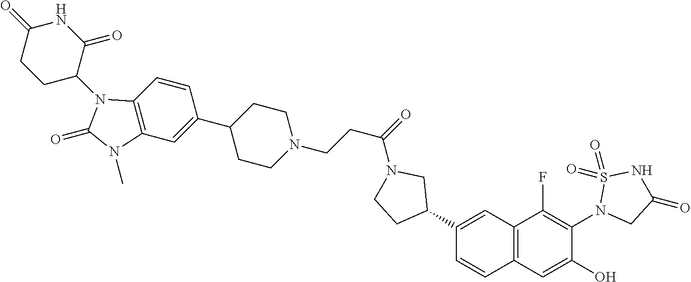 [SMILES: Cn1c(=O)n(C2CCC(=O)NC2=O)c2ccc(C3CCN(CCC(=O)N4CC[C@@H](c5ccc6cc(O)c(N7CC(=O)NS7(=O)=O)c(F)c6c5)C4)CC3)cc21] |
| 146 | 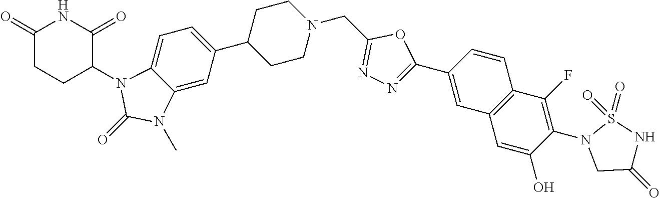 [SMILES: Cn1c(=O)n(C2CCC(=O)NC2=O)c2ccc(C3CCN(Cc4nnc(-c5ccc6c(F)c(N7CC(=O)NS7(=O)=O)c(O)cc6c5)o4)CC3)cc21] |
| 147 | 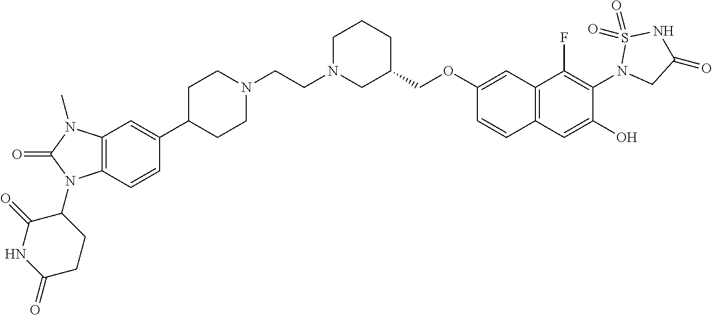 [SMILES: Cn1c(=O)n(C2CCC(=O)NC2=O)c2ccc(C3CCN(CCN4CCC[C@H](COc5ccc6cc(O)c(N7CC(=O)NS7(=O)=O)c(F)c6c5)C4)CC3)cc21] |
| 148 | 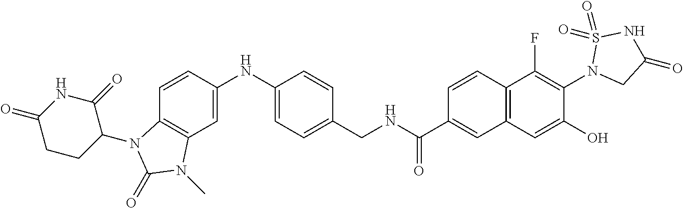 [SMILES: Cn1c(=O)n(C2CCC(=O)NC2=O)c2ccc(Nc3ccc(CNC(=O)c4ccc5c(F)c(N6CC(=O)NS6(=O)=O)c(O)cc5c4)cc3)cc21] |
| 149 | 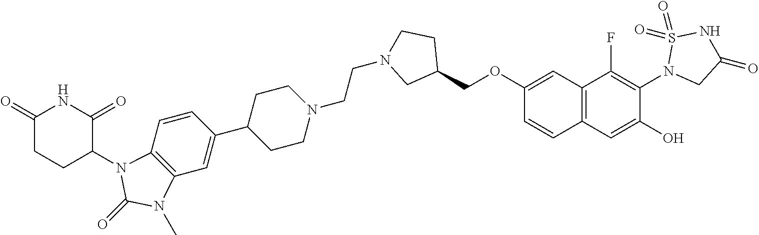 [SMILES: Cn1c(=O)n(C2CCC(=O)NC2=O)c2ccc(C3CCN(CCN4CC[C@@H](COc5ccc6cc(O)c(N7CC(=O)NS7(=O)=O)c(F)c6c5)C4)CC3)cc21] |
| 150 | 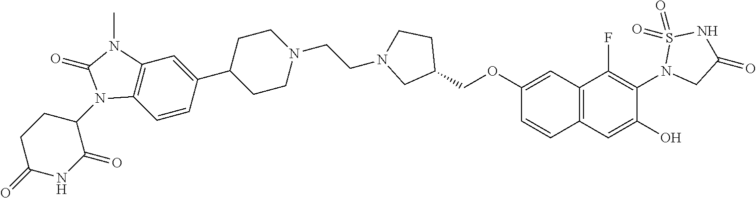 [SMILES: Cn1c(=O)n(C2CCC(=O)NC2=O)c2ccc(C3CCN(CCN4CC[C@H](COc5ccc6cc(O)c(N7CC(=O)NS7(=O)=O)c(F)c6c5)C4)CC3)cc21] |
| 151 | 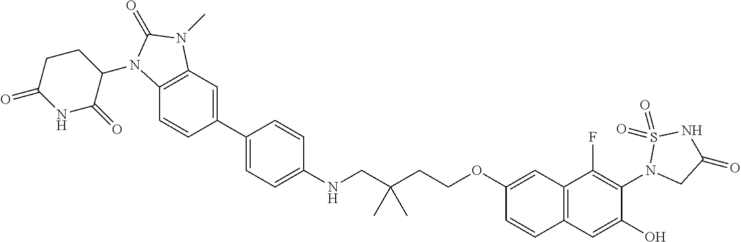 [SMILES: Cn1c(=O)n(C2CCC(=O)NC2=O)c2ccc(-c3ccc(NCC(C)(C)CCOc4ccc5cc(O)c(N6CC(=O)NS6(=O)=O)c(F)c5c4)cc3)cc21] |
| 152 | 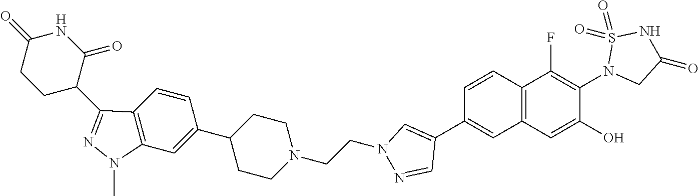 [SMILES: Cn1nc(C2CCC(=O)NC2=O)c2ccc(C3CCN(CCn4cc(-c5ccc6c(F)c(N7CC(=O)NS7(=O)=O)c(O)cc6c5)cn4)CC3)cc21] |
| 153 | 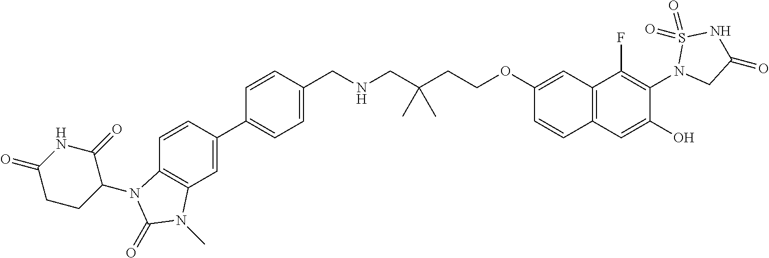 [SMILES: Cn1c(=O)n(C2CCC(=O)NC2=O)c2ccc(-c3ccc(CNCC(C)(C)CCOc4ccc5cc(O)c(N6CC(=O)NS6(=O)=O)c(F)c5c4)cc3)cc21] |
| 154 | 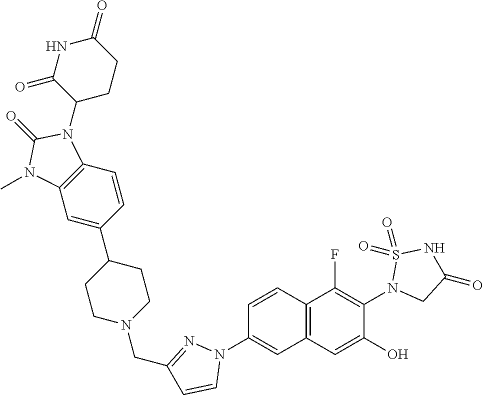 [SMILES: Cn1c(=O)n(C2CCC(=O)NC2=O)c2ccc(C3CCN(Cc4ccn(-c5ccc6c(F)c(N7CC(=O)NS7(=O)=O)c(O)cc6c5)n4)CC3)cc21] |
| 155 | 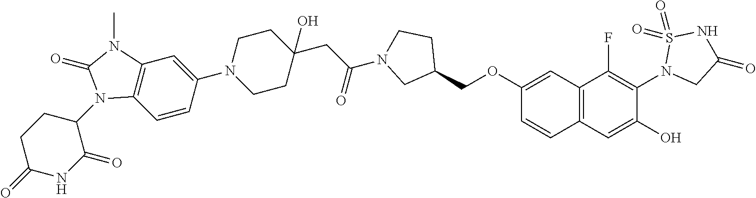 [SMILES: Cn1c(=O)n(C2CCC(=O)NC2=O)c2ccc(N3CCC(O)(CC(=O)N4CC[C@@H](COc5ccc6cc(O)c(N7CC(=O)NS7(=O)=O)c(F)c6c5)C4)CC3)cc21] |
| 156 | 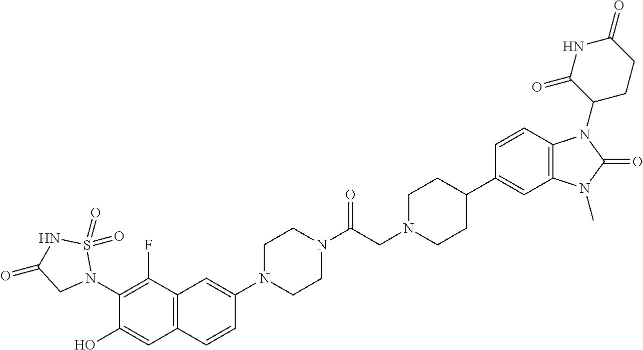 [SMILES: Cn1c(=O)n(C2CCC(=O)NC2=O)c2ccc(C3CCN(CC(=O)N4CCN(c5ccc6cc(O)c(N7CC(=O)NS7(=O)=O)c(F)c6c5)CC4)CC3)cc21] |
| 157 | 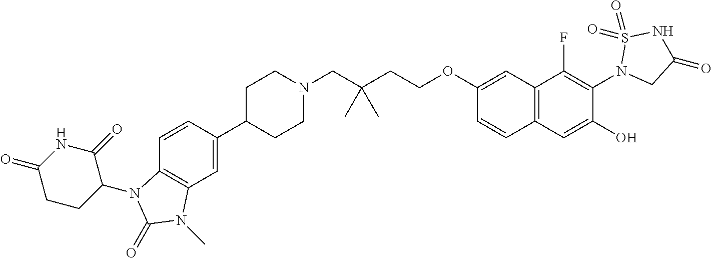 [SMILES: Cn1c(=O)n(C2CCC(=O)NC2=O)c2ccc(C3CCN(CC(C)(C)CCOc4ccc5cc(O)c(N6CC(=O)NS6(=O)=O)c(F)c5c4)CC3)cc21] |
| 158 | 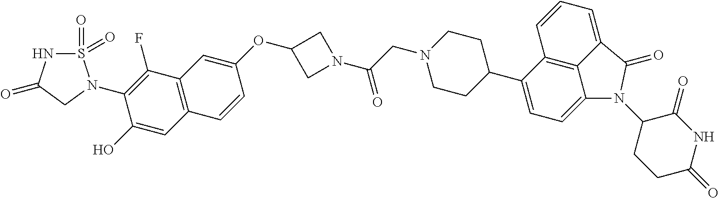 [SMILES: O=C1CCC(N2C(=O)c3cccc4c(C5CCN(CC(=O)N6CC(Oc7ccc8cc(O)c(N9CC(=O)NS9(=O)=O)c(F)c8c7)C6)CC5)ccc2c34)C(=O)N1] |
| 159 | 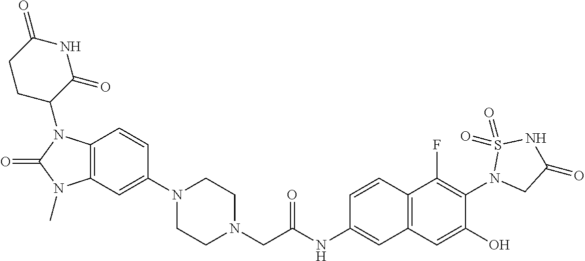 [SMILES: Cn1c(=O)n(C2CCC(=O)NC2=O)c2ccc(N3CCN(CC(=O)Nc4ccc5c(F)c(N6CC(=O)NS6(=O)=O)c(O)cc5c4)CC3)cc21] |
| 160 | 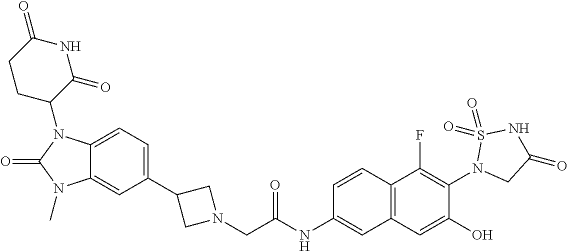 [SMILES: Cn1c(=O)n(C2CCC(=O)NC2=O)c2ccc(C3CN(CC(=O)Nc4ccc5c(F)c(N6CC(=O)NS6(=O)=O)c(O)cc5c4)C3)cc21] |
| 161 | 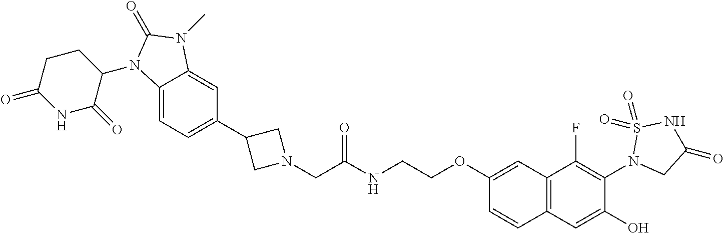 [SMILES: Cn1c(=O)n(C2CCC(=O)NC2=O)c2ccc(C3CN(CC(=O)NCCOc4ccc5cc(O)c(N6CC(=O)NS6(=O)=O)c(F)c5c4)C3)cc21] |
| 162 | 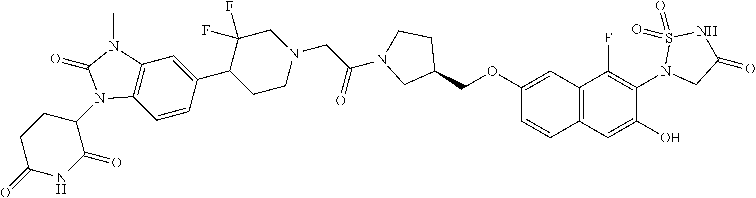 [SMILES: Cn1c(=O)n(C2CCC(=O)NC2=O)c2ccc(C3CCN(CC(=O)N4CC[C@@H](COc5ccc6cc(O)c(N7CC(=O)NS7(=O)=O)c(F)c6c5)C4)CC3(F)F)cc21] |
| 163 | 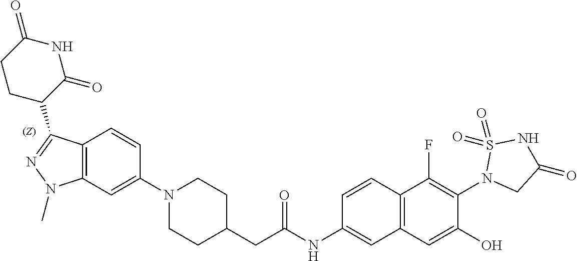 |
| 164 | 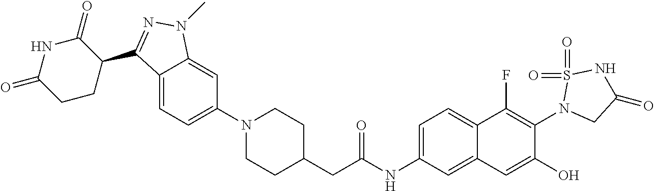 [SMILES: Cn1nc([C@H]2CCC(=O)NC2=O)c2ccc(N3CCC(CC(=O)Nc4ccc5c(F)c(N6CC(=O)NS6(=O)=O)c(O)cc5c4)CC3)cc21] |
| 165 | 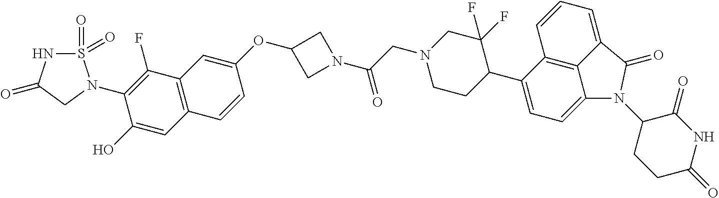 [SMILES: O=C1CCC(N2C(=O)c3cccc4c(C5CCN(CC(=O)N6CC(Oc7ccc8cc(O)c(N9CC(=O)NS9(=O)=O)c(F)c8c7)C6)CC5(F)F)ccc2c34)C(=O)N1] |
| 166 | 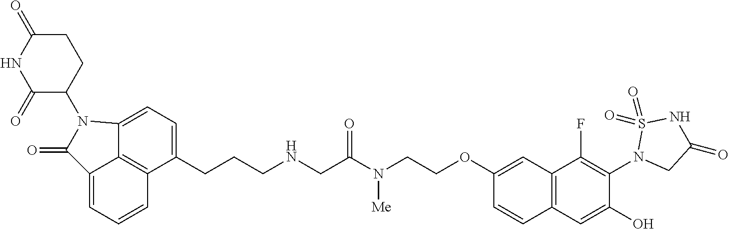 [SMILES: CN(CCOc1ccc2cc(O)c(N3CC(=O)NS3(=O)=O)c(F)c2c1)C(=O)CNCCCc1ccc2c3c(cccc13)C(=O)N2C1CCC(=O)NC1=O] |
| 167 | 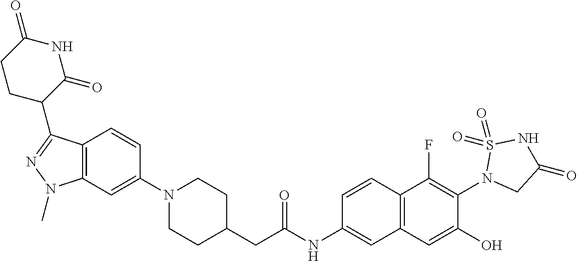 [SMILES: Cn1nc(C2CCC(=O)NC2=O)c2ccc(N3CCC(CC(=O)Nc4ccc5c(F)c(N6CC(=O)NS6(=O)=O)c(O)cc5c4)CC3)cc21] |
| 168 | 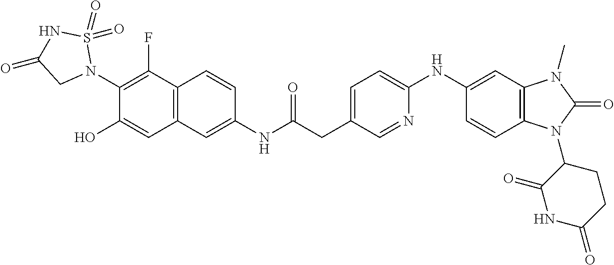 [SMILES: Cn1c(=O)n(C2CCC(=O)NC2=O)c2ccc(Nc3ccc(CC(=O)Nc4ccc5c(F)c(N6CC(=O)NS6(=O)=O)c(O)cc5c4)cn3)cc21] |
| 169 | 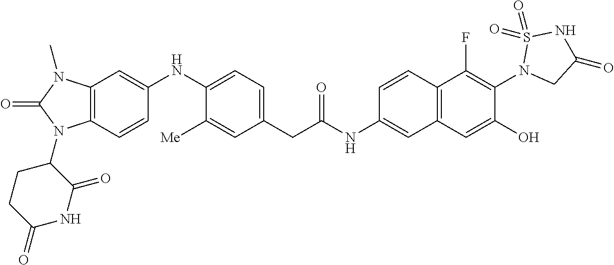 [SMILES: Cc1cc(CC(=O)Nc2ccc3c(F)c(N4CC(=O)NS4(=O)=O)c(O)cc3c2)ccc1Nc1ccc2c(c1)n(C)c(=O)n2C1CCC(=O)NC1=O] |
| 170 | 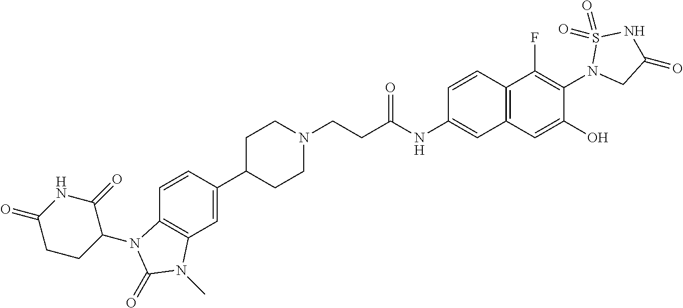 [SMILES: Cn1c(=O)n(C2CCC(=O)NC2=O)c2ccc(C3CCN(CCC(=O)Nc4ccc5c(F)c(N6CC(=O)NS6(=O)=O)c(O)cc5c4)CC3)cc21] |
| 171 | 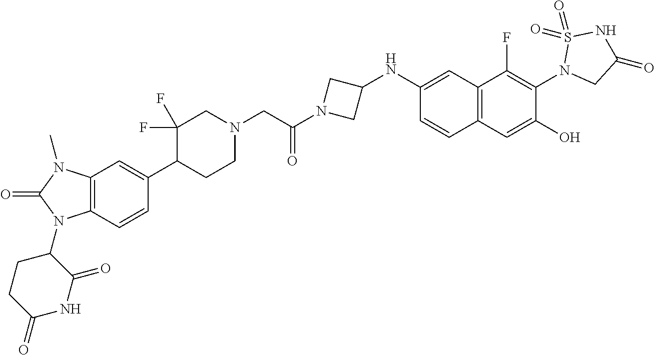 [SMILES: Cn1c(=O)n(C2CCC(=O)NC2=O)c2ccc(C3CCN(CC(=O)N4CC(Nc5ccc6cc(O)c(N7CC(=O)NS7(=O)=O)c(F)c6c5)C4)CC3(F)F)cc21] |
| 172 | 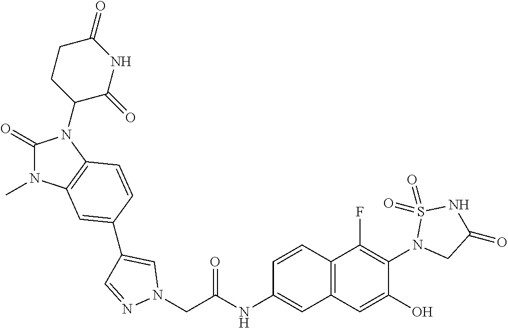 [SMILES: Cn1c(=O)n(C2CCC(=O)NC2=O)c2ccc(-c3cnn(CC(=O)Nc4ccc5c(F)c(N6CC(=O)NS6(=O)=O)c(O)cc5c4)c3)cc21] |
| 173 | 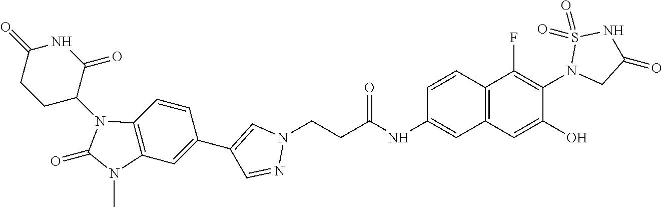 [SMILES: Cn1c(=O)n(C2CCC(=O)NC2=O)c2ccc(-c3cnn(CCC(=O)Nc4ccc5c(F)c(N6CC(=O)NS6(=O)=O)c(O)cc5c4)c3)cc21] |
| 174 | 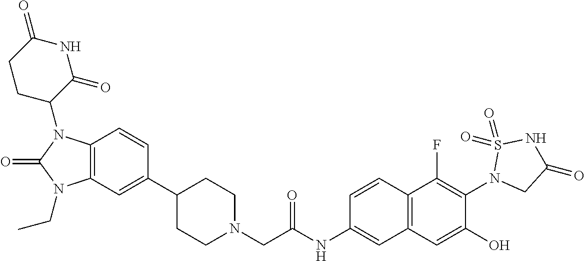 [SMILES: CCn1c(=O)n(C2CCC(=O)NC2=O)c2ccc(C3CCN(CC(=O)Nc4ccc5c(F)c(N6CC(=O)NS6(=O)=O)c(O)cc5c4)CC3)cc21] |
| 175 | 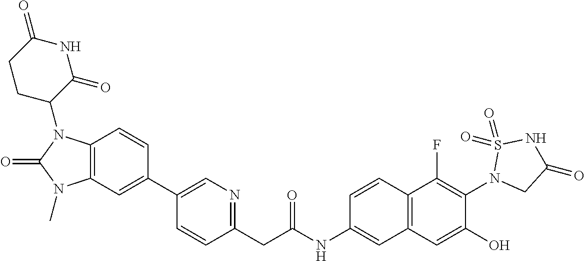 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)nc3)cc21] |
| 176 | 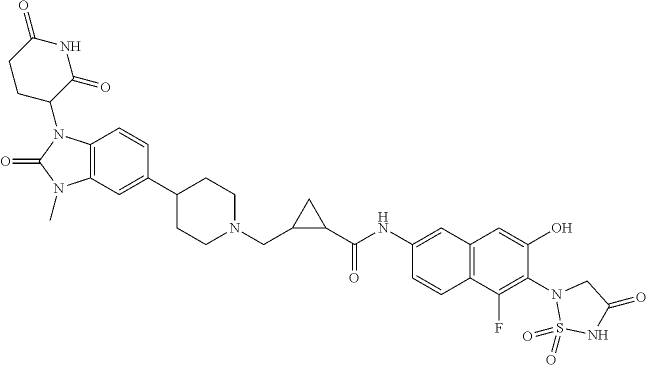 [SMILES: Cn1c(=O)n(C2CCC(=O)NC2=O)c2ccc(C3CCN(CC4CC4C(=O)Nc4ccc5c(F)c(N6CC(=O)NS6(=O)=O)c(O)cc5c4)CC3)cc21] |
| 177 | 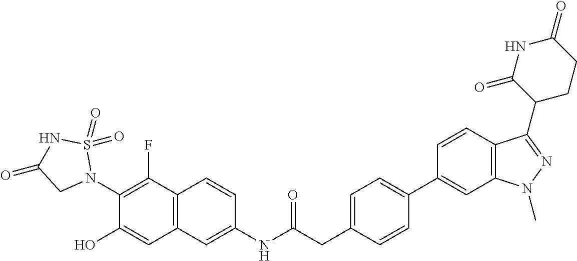 [SMILES: Cn1nc(C2CCC(=O)NC2=O)c2ccc(-c3ccc(CC(=O)Nc4ccc5c(F)c(N6CC(=O)NS6(=O)=O)c(O)cc5c4)cc3)cc21] |
| 178 | 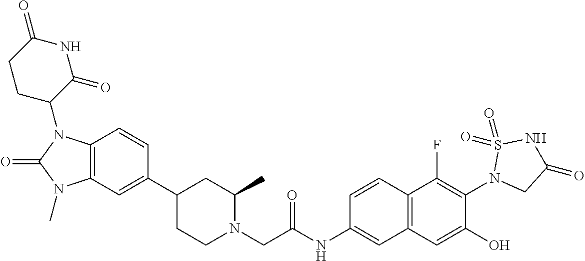 [SMILES: C[C@@H]1CC(c2ccc3c(c2)n(C)c(=O)n3C2CCC(=O)NC2=O)CCN1CC(=O)Nc1ccc2c(F)c(N3CC(=O)NS3(=O)=O)c(O)cc2c1] |
| 179 | 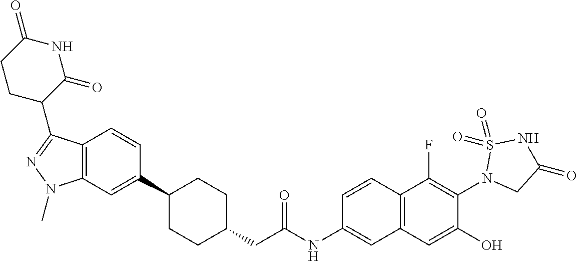 [SMILES: Cn1nc(C2CCC(=O)NC2=O)c2ccc([C@H]3CC[C@H](CC(=O)Nc4ccc5c(F)c(N6CC(=O)NS6(=O)=O)c(O)cc5c4)CC3)cc21] |
| 180 | 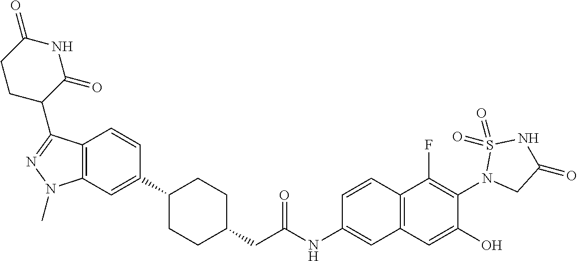 [SMILES: Cn1nc(C2CCC(=O)NC2=O)c2ccc([C@H]3CC[C@@H](CC(=O)Nc4ccc5c(F)c(N6CC(=O)NS6(=O)=O)c(O)cc5c4)CC3)cc21] |
| 181 | 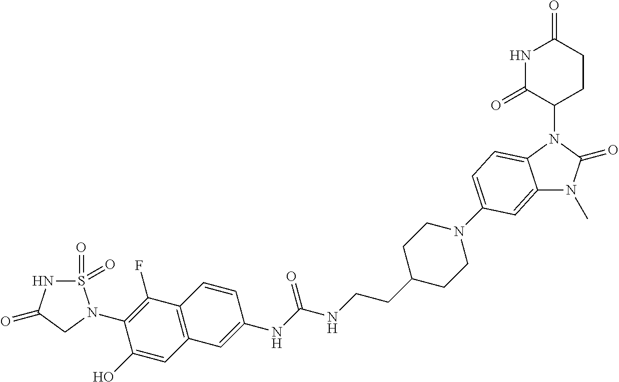 [SMILES: Cn1c(=O)n(C2CCC(=O)NC2=O)c2ccc(N3CCC(CCNC(=O)Nc4ccc5c(F)c(N6CC(=O)NS6(=O)=O)c(O)cc5c4)CC3)cc21] |
| 182 | 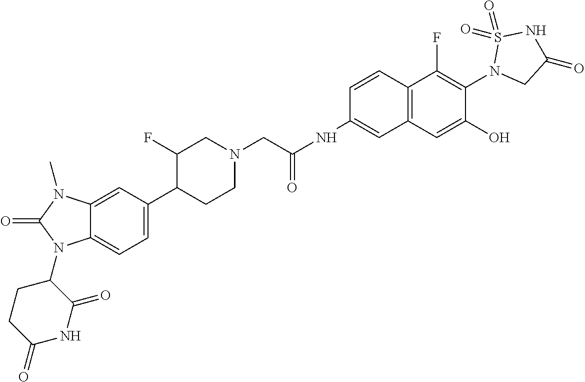 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3F)cc21] |
| 183 | 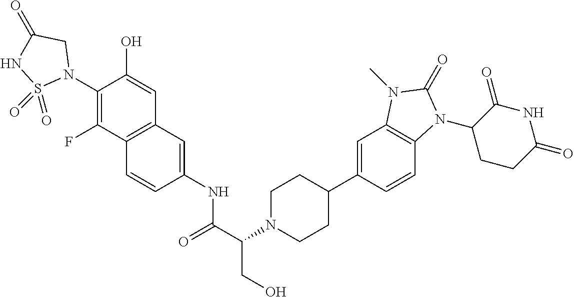 [SMILES: Cn1c(=O)n(C2CCC(=O)NC2=O)c2ccc(C3CCN([C@H](CO)C(=O)Nc4ccc5c(F)c(N6CC(=O)NS6(=O)=O)c(O)cc5c4)CC3)cc21] |
| 184 | 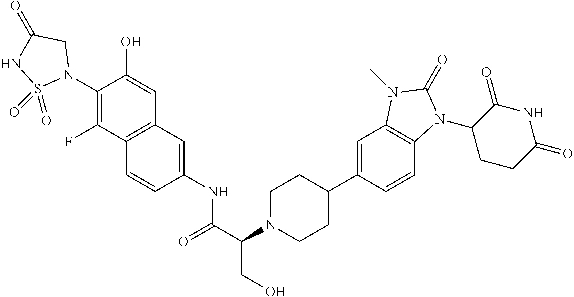 [SMILES: Cn1c(=O)n(C2CCC(=O)NC2=O)c2ccc(C3CCN([C@@H](CO)C(=O)Nc4ccc5c(F)c(N6CC(=O)NS6(=O)=O)c(O)cc5c4)CC3)cc21] |
| 185 | 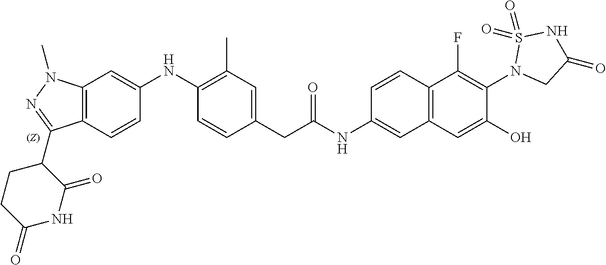 [SMILES: Cc1cc(CC(=O)Nc2ccc3c(F)c(N4CC(=O)NS4(=O)=O)c(O)cc3c2)ccc1Nc1ccc2c(C3CCC(=O)NC3=O)nn(C)c2c1] |

In some embodiments, a compound of Formula (I) is selected from a compound set forth in Table 2.

TABLE 2
Further exemplary compounds of the disclosure.

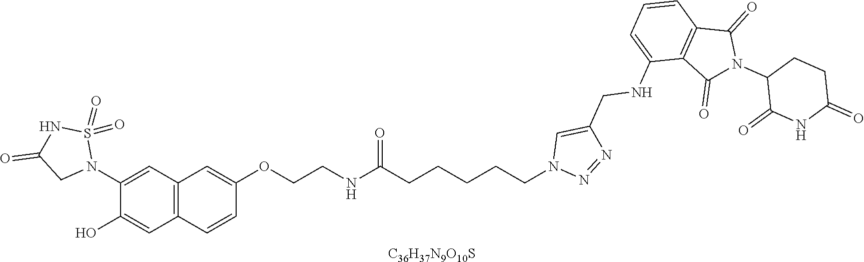 [SMILES: "C36H37N9O10S" ; O=C(CCCCCn1cc(CNc2cccc3c2C(=O)N(C2CCC(=O)NC2=O)C3=O)nn1)NCCOc1ccc2cc(O)c(N3CC(=O)NS3(=O)=O)cc2c1]

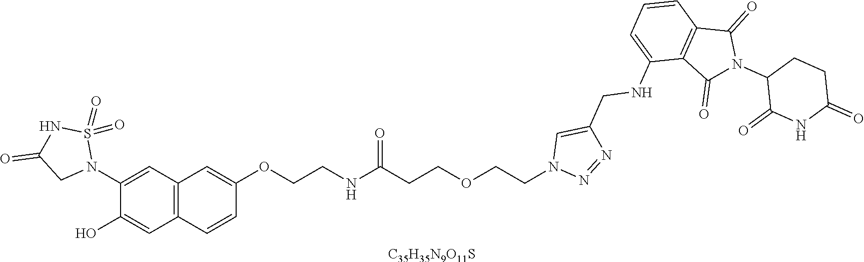 [SMILES: "C35H35N9O11S" ; O=C(CCOCCn1cc(CNc2cccc3c2C(=O)N(C2CCC(=O)NC2=O)C3=O)nn1)NCCOc1ccc2cc(O)c(N3CC(=O)NS3(=O)=O)cc2c1]

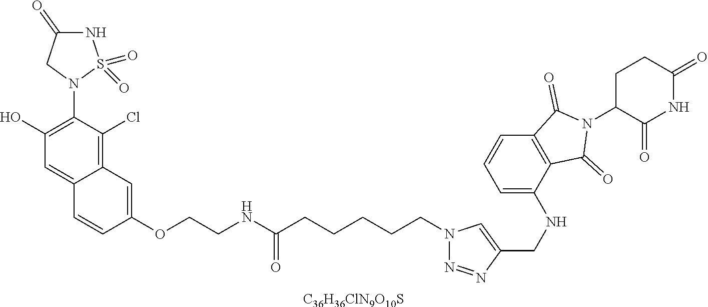 [SMILES: "C36H36ClN9O10S" ; O=C(CCCCCn1cc(CNc2cccc3c2C(=O)N(C2CCC(=O)NC2=O)C3=O)nn1)NCCOc1ccc2cc(O)c(N3CC(=O)NS3(=O)=O)c(Cl)c2c1]

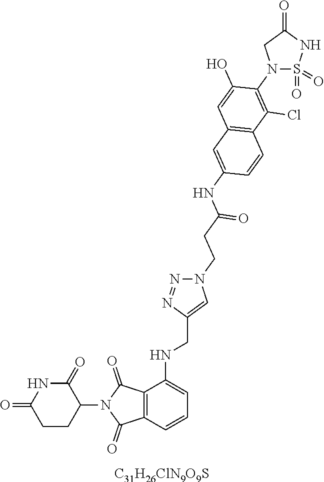

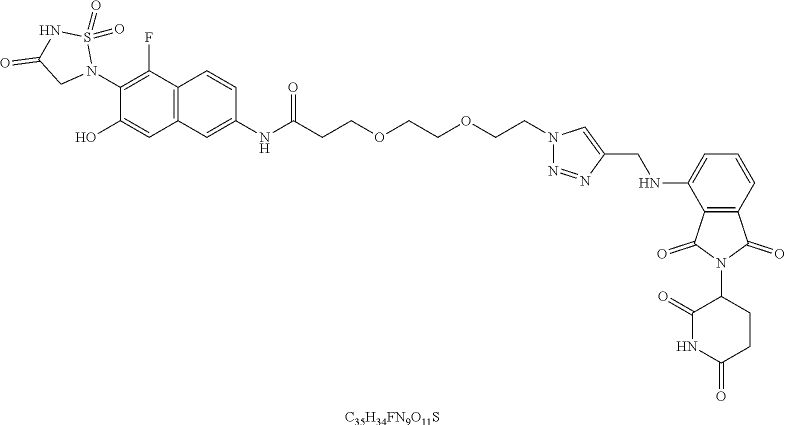 [SMILES: O=C1CCC(N2C(=O)c3cccc(NCc4cn(CCOCCOCCC(=O)Nc5ccc6c(F)c(N7CC(=O)NS7(=O)=O)c(O)cc6c5)nn4)c3C2=O)C(=O)N1 ; "C35H34FN9O11S"]

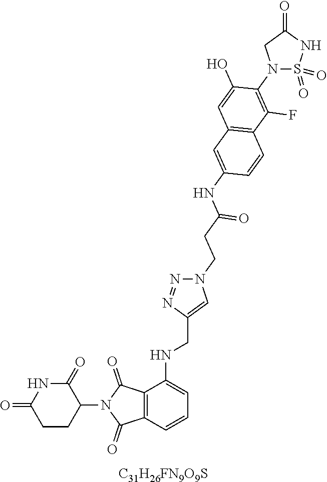

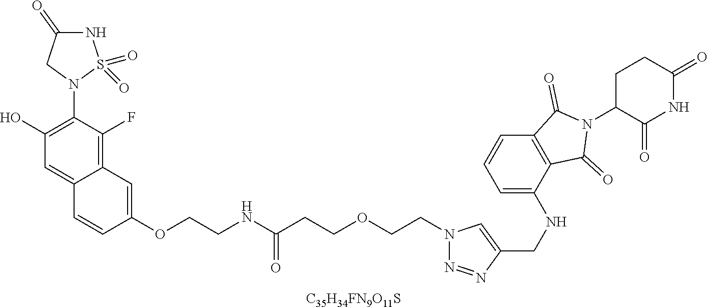

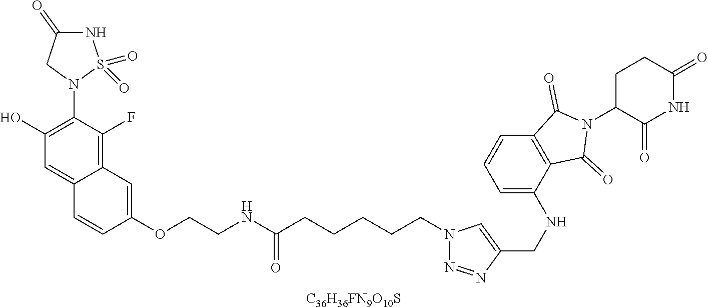

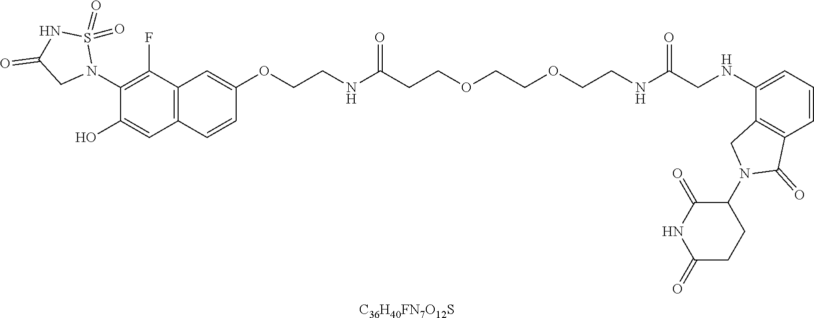 [SMILES: O=C(CCOCCOCCNC(=O)CNc1cccc2c1CN(C1CCC(=O)NC1=O)C2=O)NCCOc1ccc2cc(O)c(N3CC(=O)NS3(=O)=O)c(F)c2c1 ; "C36H40FN7O12S"]

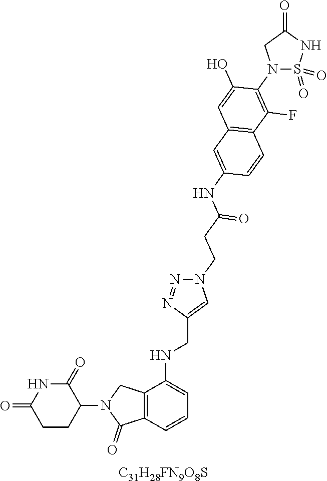

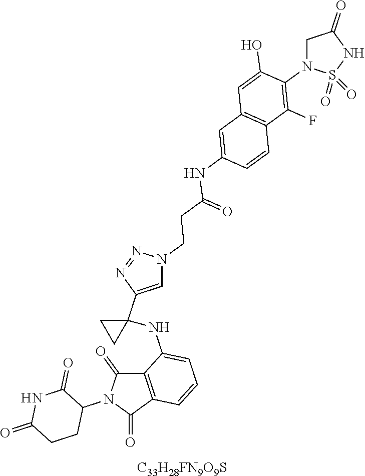

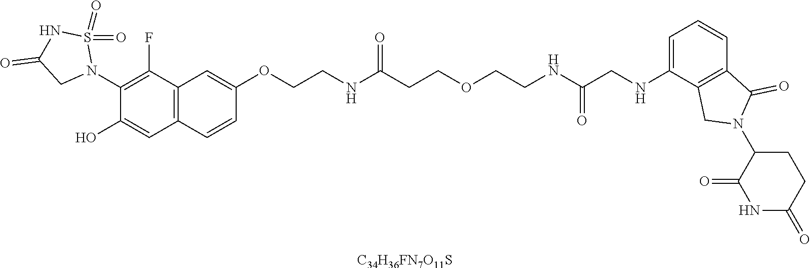 [SMILES: O=C(CCOCCNC(=O)CNc1cccc2c1CN(C1CCC(=O)NC1=O)C2=O)NCCOc1ccc2cc(O)c(N3CC(=O)NS3(=O)=O)c(F)c2c1 ; "C34H36FN7O11S"]

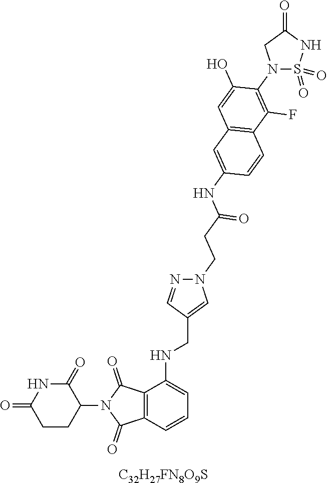

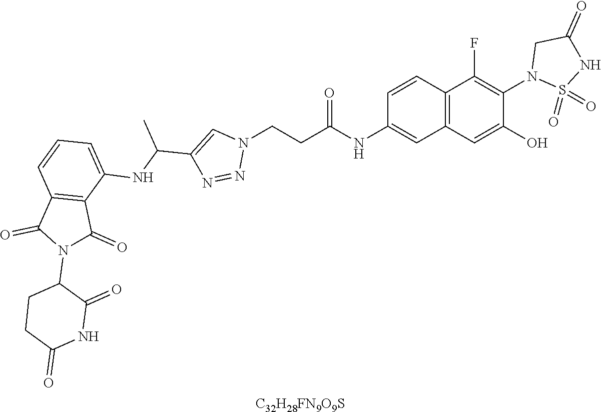 [SMILES: CC(Nc1cccc2c1C(=O)N(C1CCC(=O)NC1=O)C2=O)c1cn(CCC(=O)Nc2ccc3c(F)c(N4CC(=O)NS4(=O)=O)c(O)cc3c2)nn1 ; "C32H28FN9O9S"]

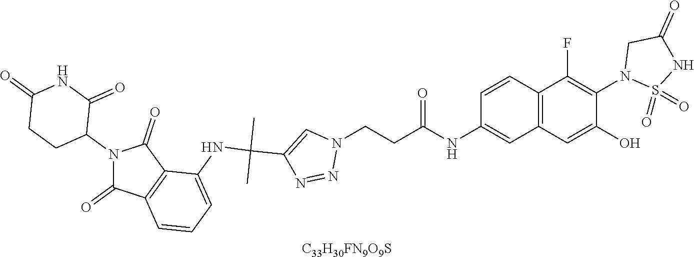 [SMILES: CC(C)(Nc1cccc2c1C(=O)N(C1CCC(=O)NC1=O)C2=O)c1cn(CCC(=O)Nc2ccc3c(F)c(N4CC(=O)NS4(=O)=O)c(O)cc3c2)nn1 ; "C33H30FN9O9S"]

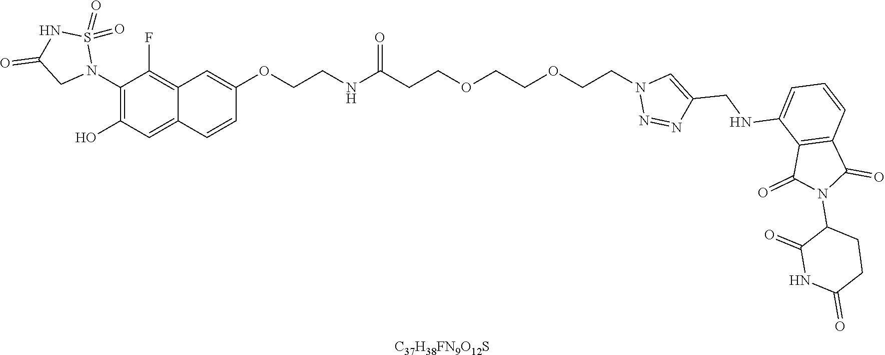 [SMILES: O=C(CCOCCOCCn1cc(CNc2cccc3c2C(=O)N(C2CCC(=O)NC2=O)C3=O)nn1)NCCOc1ccc2cc(O)c(N3CC(=O)NS3(=O)=O)c(F)c2c1 ; "C37H38FN9O12S"]

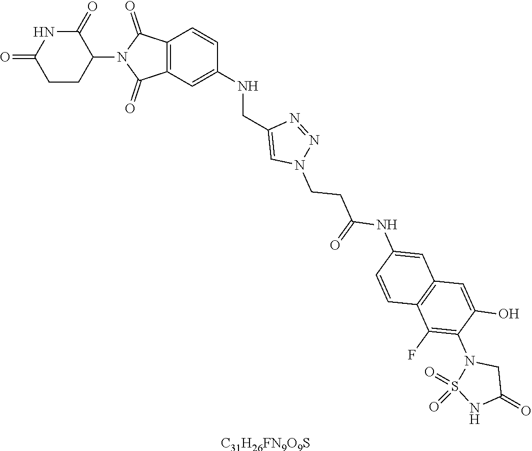 [SMILES: O=C1CCC(N2C(=O)c3ccc(NCc4cn(CCC(=O)Nc5ccc6c(F)c(N7CC(=O)NS7(=O)=O)c(O)cc6c5)nn4)cc3C2=O)C(=O)N1 ; "C31H26FN9O9S"]

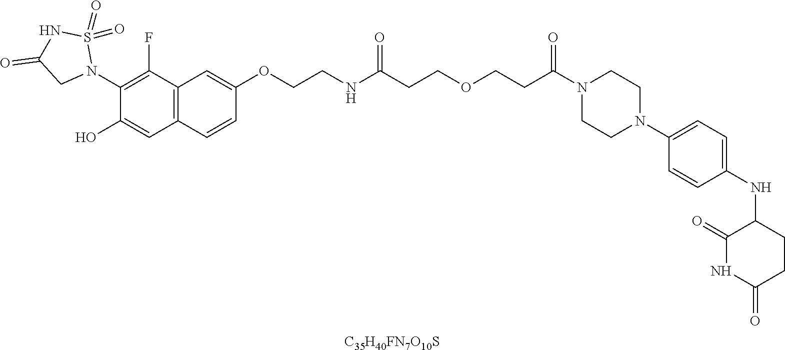 [SMILES: O=C(CCOCCC(=O)N1CCN(c2ccc(NC3CCC(=O)NC3=O)cc2)CC1)NCCOc1ccc2cc(O)c(N3CC(=O)NS3(=O)=O)c(F)c2c1 ; "C35H40FN7O10S"]

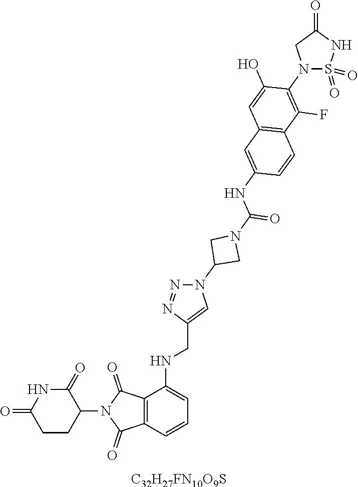

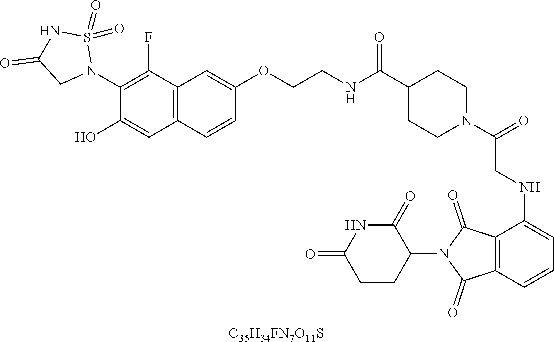 [SMILES: O=C1CCC(N2C(=O)c3cccc(NCC(=O)N4CCC(C(=O)NCCOc5ccc6cc(O)c(N7CC(=O)NS7(=O)=O)c(F)c6c5)CC4)c3C2=O)C(=O)N1 ; "C35H34FN7O11S"]

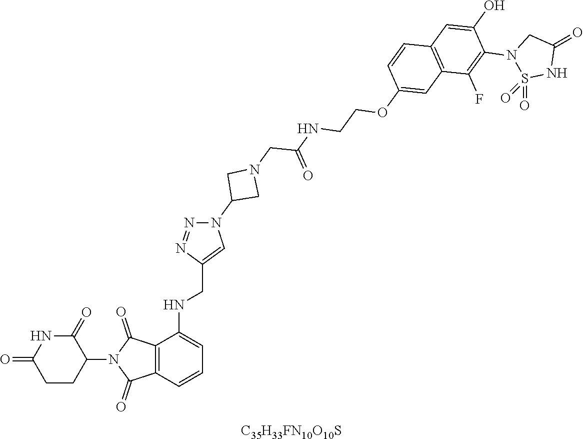 [SMILES: O=C(CN1CC(n2cc(CNc3cccc4c3C(=O)N(C3CCC(=O)NC3=O)C4=O)nn2)C1)NCCOc1ccc2cc(O)c(N3CC(=O)NS3(=O)=O)c(F)c2c1 ; "C35H33FN10O10S"]

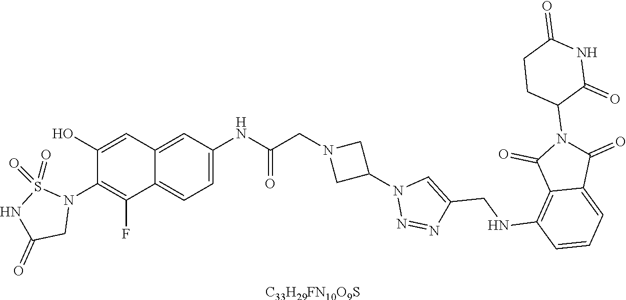 [SMILES: O=C1CCC(N2C(=O)c3cccc(NCc4cn(C5CN(CC(=O)Nc6ccc7c(F)c(N8CC(=O)NS8(=O)=O)c(O)cc7c6)C5)nn4)c3C2=O)C(=O)N1 ; "C33H29FN10O9S"]

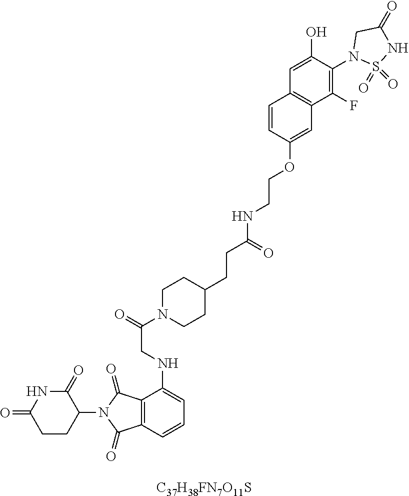 [SMILES: O=C(CCC1CCN(C(=O)CNc2cccc3c2C(=O)N(C2CCC(=O)NC2=O)C3=O)CC1)NCCOc1ccc2cc(O)c(N3CC(=O)NS3(=O)=O)c(F)c2c1 ; "C37H38FN7O11S"]

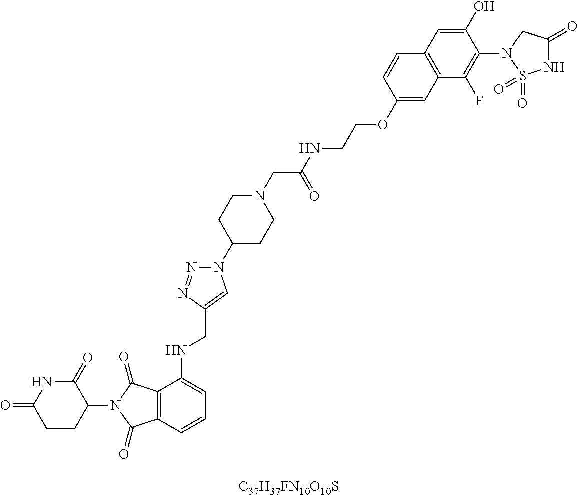 [SMILES: O=C(CN1CCC(n2cc(CNc3cccc4c3C(=O)N(C3CCC(=O)NC3=O)C4=O)nn2)CC1)NCCOc1ccc2cc(O)c(N3CC(=O)NS3(=O)=O)c(F)c2c1 ; "C37H37FN10O10S"]

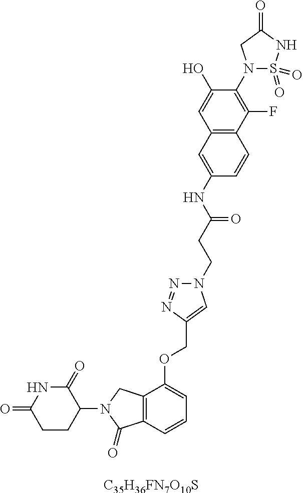

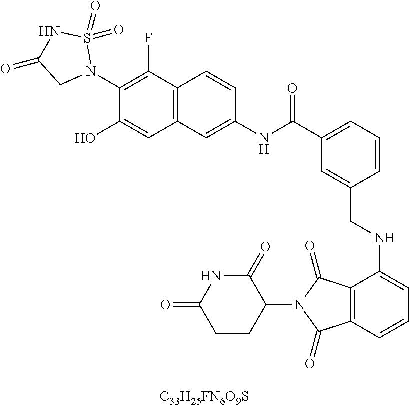 [SMILES: O=C1CCC(N2C(=O)c3cccc(NCc4cccc(C(=O)Nc5ccc6c(F)c(N7CC(=O)NS7(=O)=O)c(O)cc6c5)c4)c3C2=O)C(=O)N1 ; "C33H25FN6O9S"]

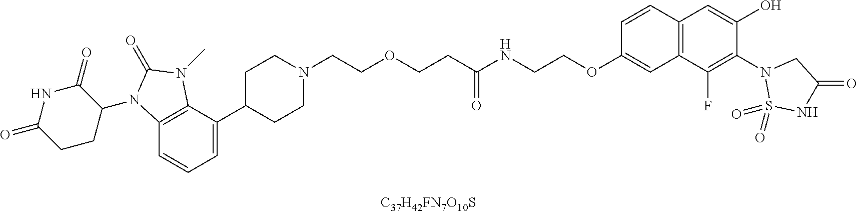 [SMILES: Cn1c(=O)n(C2CCC(=O)NC2=O)c2cccc(C3CCN(CCOCCC(=O)NCCOc4ccc5cc(O)c(N6CC(=O)NS6(=O)=O)c(F)c5c4)CC3)c21 ; "C37H42FN7O10S"]

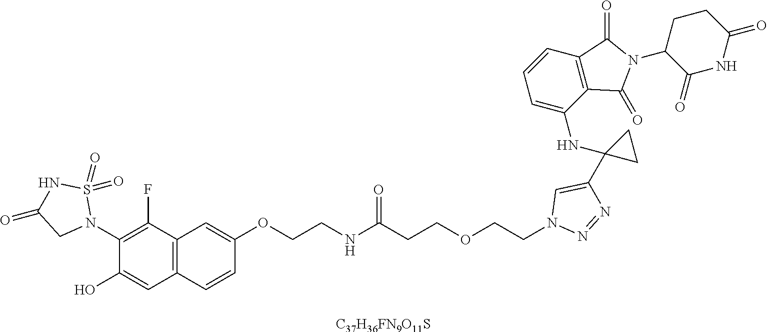 [SMILES: O=C(CCOCCn1cc(C2(Nc3cccc4c3C(=O)N(C3CCC(=O)NC3=O)C4=O)CC2)nn1)NCCOc1ccc2cc(O)c(N3CC(=O)NS3(=O)=O)c(F)c2c1 ; "C37H36FN9O11S"]

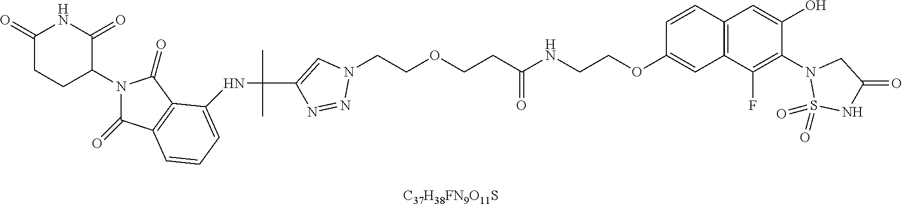 [SMILES: CC(C)(Nc1cccc2c1C(=O)N(C1CCC(=O)NC1=O)C2=O)c1cn(CCOCCC(=O)NCCOc2ccc3cc(O)c(N4CC(=O)NS4(=O)=O)c(F)c3c2)nn1 ; "C37H38FN9O11S"]

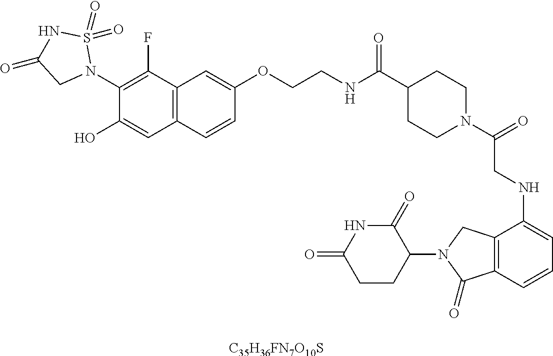 [SMILES: O=C1CCC(N2Cc3c(NCC(=O)N4CCC(C(=O)NCCOc5ccc6cc(O)c(N7CC(=O)NS7(=O)=O)c(F)c6c5)CC4)cccc3C2=O)C(=O)N1 ; "C35H36FN7O10S"]

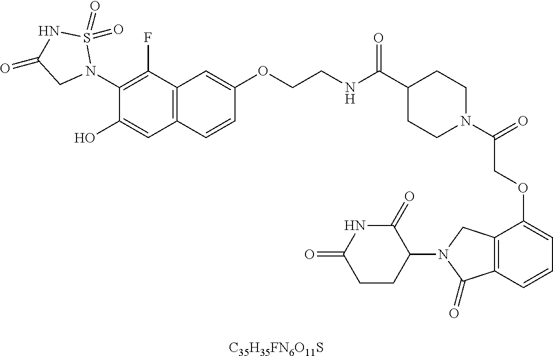 [SMILES: O=C1CCC(N2Cc3c(OCC(=O)N4CCC(C(=O)NCCOc5ccc6cc(O)c(N7CC(=O)NS7(=O)=O)c(F)c6c5)CC4)cccc3C2=O)C(=O)N1 ; "C35H35FN6O11S"]

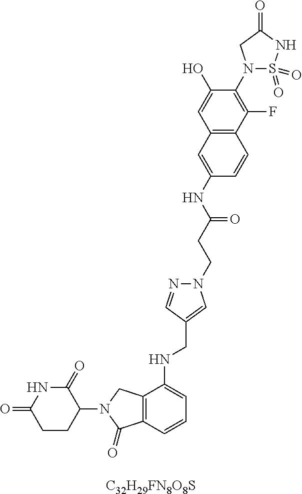

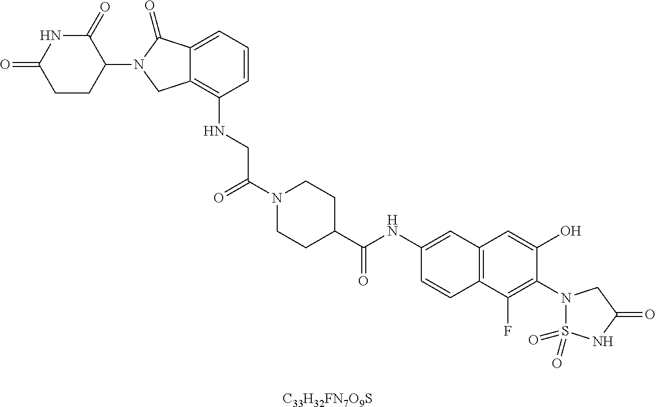 [SMILES: O=C1CCC(N2Cc3c(NCC(=O)N4CCC(C(=O)Nc5ccc6c(F)c(N7CC(=O)NS7(=O)=O)c(O)cc6c5)CC4)cccc3C2=O)C(=O)N1 ; "C33H32FN7O9S"]

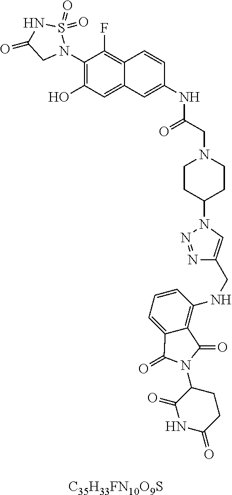 [SMILES: O=C1CCC(N2C(=O)c3cccc(NCc4cn(C5CCN(CC(=O)Nc6ccc7c(F)c(N8CC(=O)NS8(=O)=O)c(O)cc7c6)CC5)nn4)c3C2=O)C(=O)N1 ; "C35H33FN10O9S"]

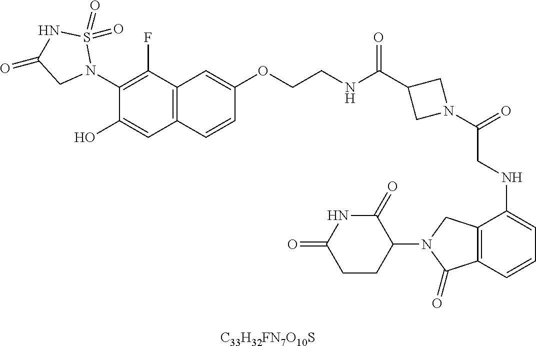 [SMILES: O=C1CCC(N2Cc3c(NCC(=O)N4CC(C(=O)NCCOc5ccc6cc(O)c(N7CC(=O)NS7(=O)=O)c(F)c6c5)C4)cccc3C2=O)C(=O)N1 ; "C33H32FN7O10S"]

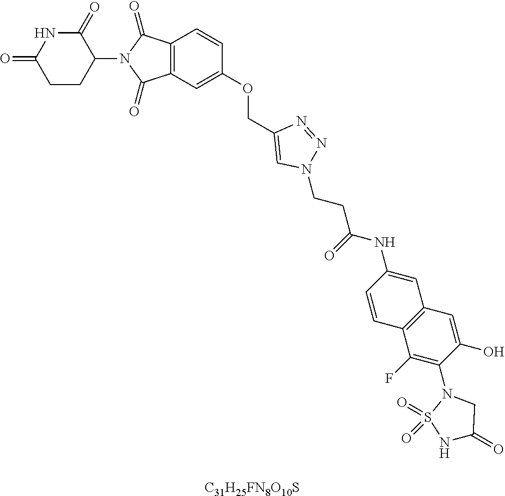 [SMILES: O=C1CCC(N2C(=O)c3ccc(OCc4cn(CCC(=O)Nc5ccc6c(F)c(N7CC(=O)NS7(=O)=O)c(O)cc6c5)nn4)cc3C2=O)C(=O)N1 ; "C31H25FN8O10S"]

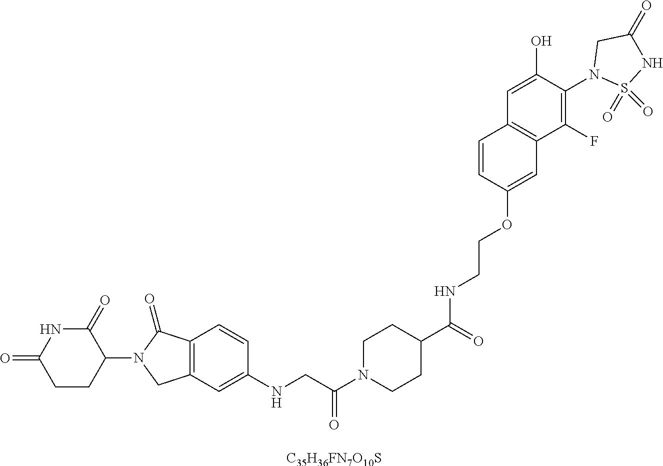 [SMILES: O=C1CCC(N2Cc3cc(NCC(=O)N4CCC(C(=O)NCCOc5ccc6cc(O)c(N7CC(=O)NS7(=O)=O)c(F)c6c5)CC4)ccc3C2=O)C(=O)N1 ; "C35H36FN7O10S"]

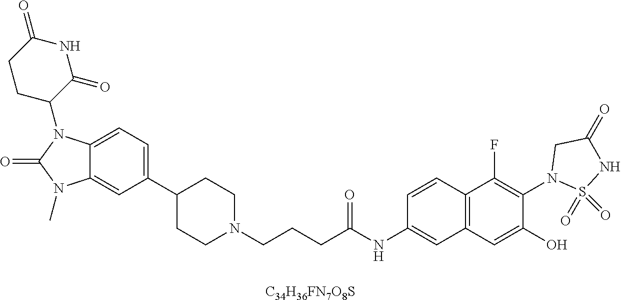 [SMILES: Cn1c(=O)n(C2CCC(=O)NC2=O)c2ccc(C3CCN(CCCC(=O)Nc4ccc5c(F)c(N6CC(=O)NS6(=O)=O)c(O)cc5c4)CC3)cc21 ; "C34H36FN7O8S"]

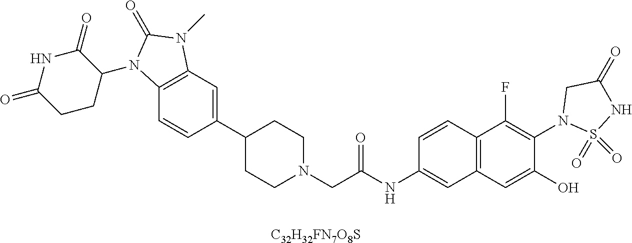 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3)cc21 ; "C32H32FN7O8S"]

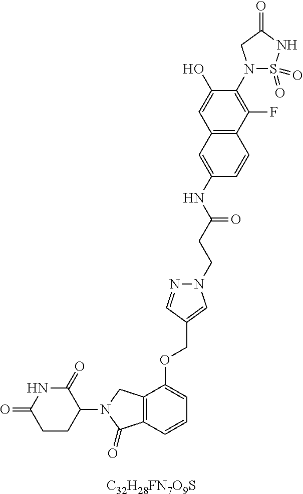

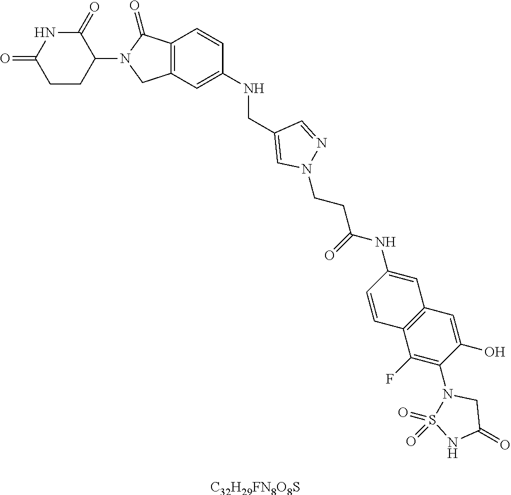 [SMILES: O=C1CCC(N2Cc3cc(NCc4cnn(CCC(=O)Nc5ccc6c(F)c(N7CC(=O)NS7(=O)=O)c(O)cc6c5)c4)ccc3C2=O)C(=O)N1 ; "C32H29FN8O8S"]

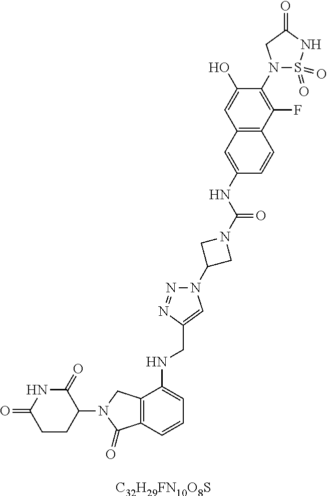 [SMILES: O=C1CCC(N2Cc3c(NCc4cn(C5CN(C(=O)Nc6ccc7c(F)c(N8CC(=O)NS8(=O)=O)c(O)cc7c6)C5)nn4)cccc3C2=O)C(=O)N1 ; "C32H29FN10O8S"]

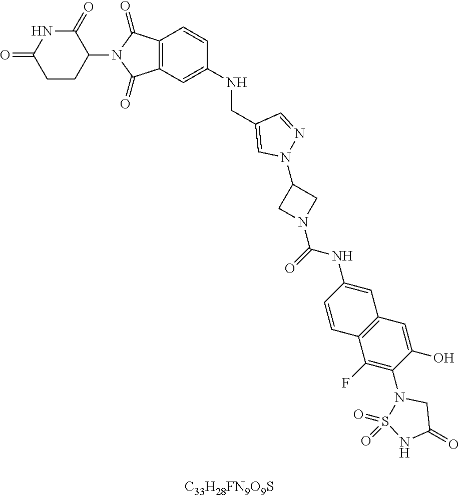 [SMILES: O=C1CCC(N2C(=O)c3ccc(NCc4cnn(C5CN(C(=O)Nc6ccc7c(F)c(N8CC(=O)NS8(=O)=O)c(O)cc7c6)C5)c4)cc3C2=O)C(=O)N1 ; "C33H28FN9O9S"]

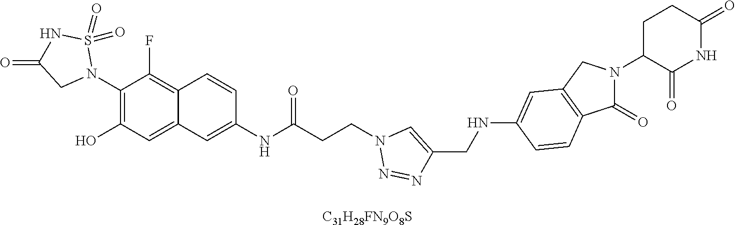 [SMILES: O=C1CCC(N2Cc3cc(NCc4cn(CCC(=O)Nc5ccc6c(F)c(N7CC(=O)NS7(=O)=O)c(O)cc6c5)nn4)ccc3C2=O)C(=O)N1 ; "C31H28FN9O8S"]

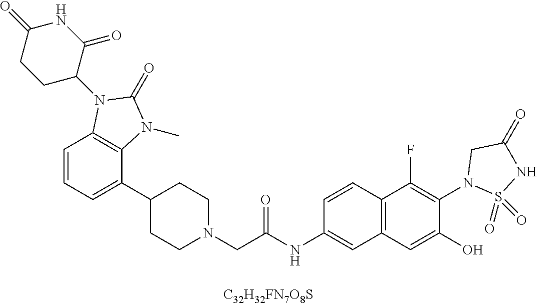

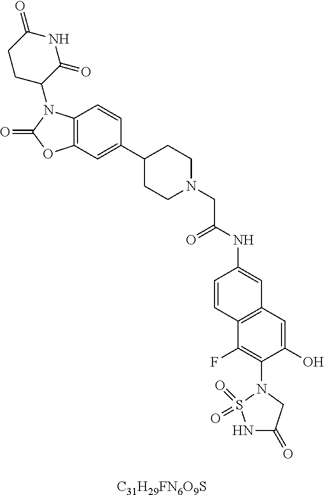 [SMILES: O=C1CCC(n2c(=O)oc3cc(C4CCN(CC(=O)Nc5ccc6c(F)c(N7CC(=O)NS7(=O)=O)c(O)cc6c5)CC4)ccc32)C(=O)N1 ; "C31H29FN6O9S"]

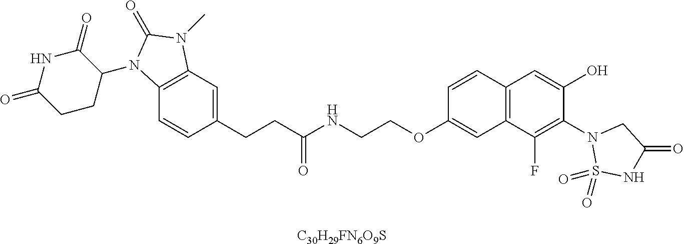 [SMILES: Cn1c(=O)n(C2CCC(=O)NC2=O)c2ccc(CCC(=O)NCCOc3ccc4cc(O)c(N5CC(=O)NS5(=O)=O)c(F)c4c3)cc21 ; "C30H29FN6O9S"]

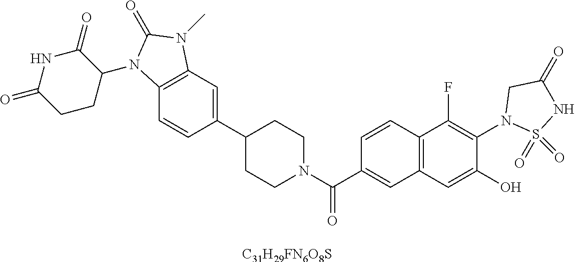 [SMILES: Cn1c(=O)n(C2CCC(=O)NC2=O)c2ccc(C3CCN(C(=O)c4ccc5c(F)c(N6CC(=O)NS6(=O)=O)c(O)cc5c4)CC3)cc21 ; "C31H29FN6O8S"]

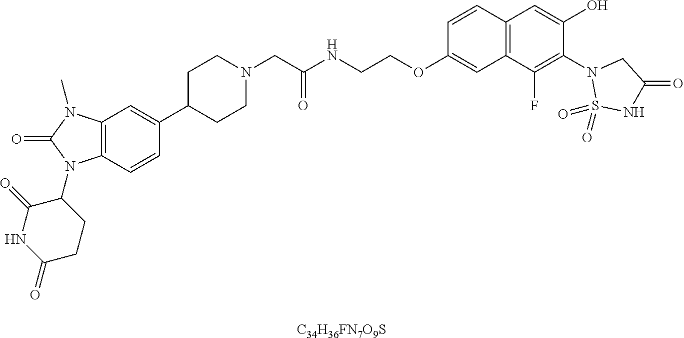 [SMILES: Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21 ; "C34H36FN7O9S"]

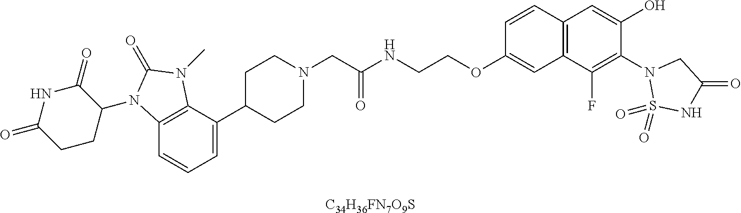 [SMILES: Cn1c(=O)n(C2CCC(=O)NC2=O)c2cccc(C3CCN(CC(=O)NCCOc4ccc5cc(O)c(N6CC(=O)NS6(=O)=O)c(F)c5c4)CC3)c21 ; "C34H36FN7O9S"]

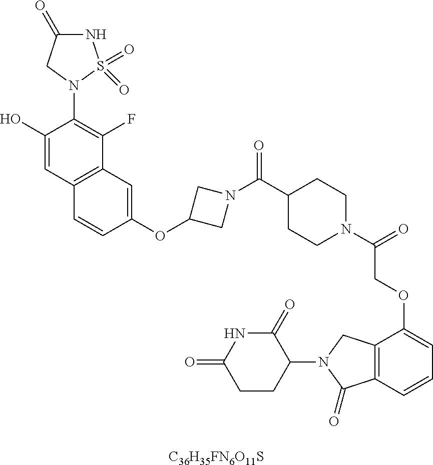 [SMILES: O=C1CCC(N2Cc3c(OCC(=O)N4CCC(C(=O)N5CC(Oc6ccc7cc(O)c(N8CC(=O)NS8(=O)=O)c(F)c7c6)C5)CC4)cccc3C2=O)C(=O)N1 ; "C36H35FN6O11S"]

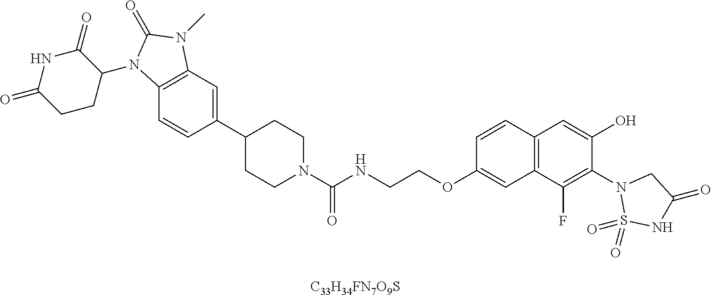 [SMILES: Cn1c(=O)n(C2CCC(=O)NC2=O)c2ccc(C3CCN(C(=O)NCCOc4ccc5cc(O)c(N6CC(=O)NS6(=O)=O)c(F)c5c4)CC3)cc21 ; "C33H34FN7O9S"]

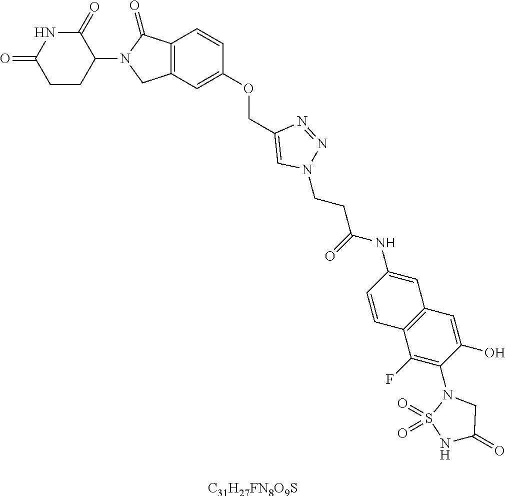 [SMILES: O=C1CCC(N2Cc3cc(OCc4cn(CCC(=O)Nc5ccc6c(F)c(N7CC(=O)NS7(=O)=O)c(O)cc6c5)nn4)ccc3C2=O)C(=O)N1 ; "C31H27FN8O9S"]

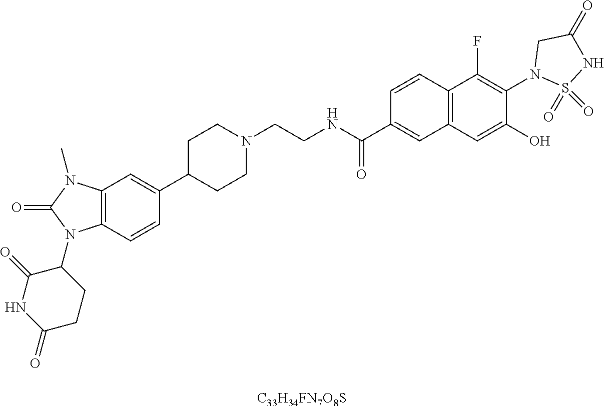 [SMILES: Cn1c(=O)n(C2CCC(=O)NC2=O)c2ccc(C3CCN(CCNC(=O)c4ccc5c(F)c(N6CC(=O)NS6(=O)=O)c(O)cc5c4)CC3)cc21 ; "C33H34FN7O8S"]

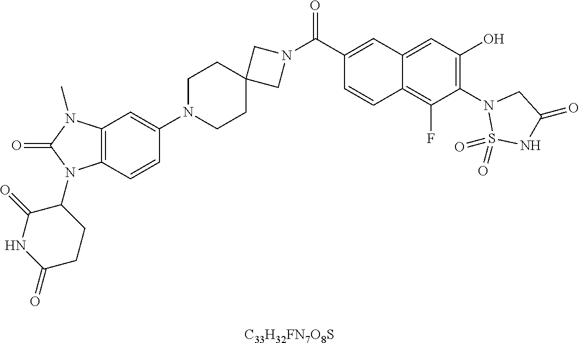 [SMILES: Cn1c(=O)n(C2CCC(=O)NC2=O)c2ccc(N3CCC4(CC3)CN(C(=O)c3ccc5c(F)c(N6CC(=O)NS6(=O)=O)c(O)cc5c3)C4)cc21 ; "C33H32FN7O8S"]

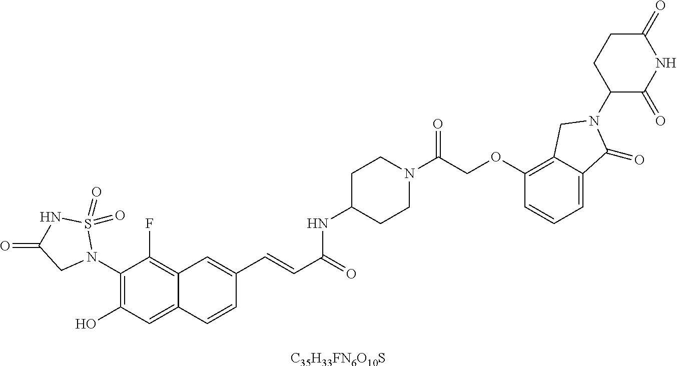 [SMILES: O=C(/C=C/c1ccc2cc(O)c(N3CC(=O)NS3(=O)=O)c(F)c2c1)NC1CCN(C(=O)COc2cccc3c2CN(C2CCC(=O)NC2=O)C3=O)CC1 ; "C35H33FN6O10S"]

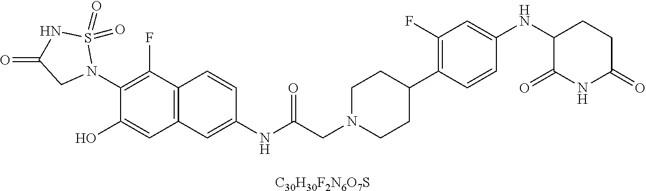 [SMILES: O=C1CCC(Nc2ccc(C3CCN(CC(=O)Nc4ccc5c(F)c(N6CC(=O)NS6(=O)=O)c(O)cc5c4)CC3)c(F)c2)C(=O)N1 ; "C30H30F2N6O7S"]

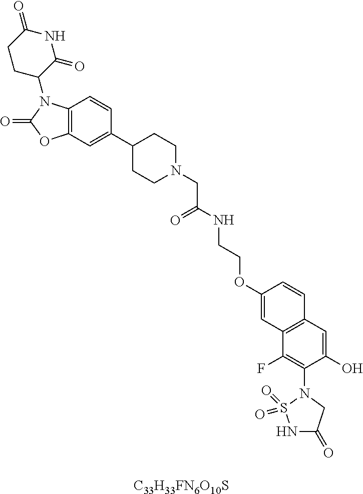 [SMILES: O=C(CN1CCC(c2ccc3c(c2)oc(=O)n3C2CCC(=O)NC2=O)CC1)NCCOc1ccc2cc(O)c(N3CC(=O)NS3(=O)=O)c(F)c2c1 ; "C33H33FN6O10S"]

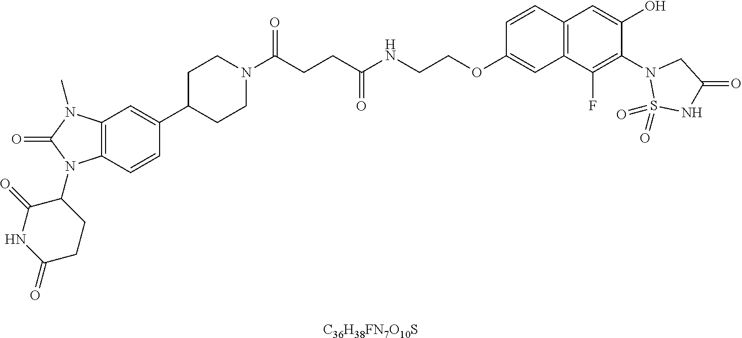 [SMILES: Cn1c(=O)n(C2CCC(=O)NC2=O)c2ccc(C3CCN(C(=O)CCC(=O)NCCOc4ccc5cc(O)c(N6CC(=O)NS6(=O)=O)c(F)c5c4)CC3)cc21 ; "C36H38FN7O10S"]

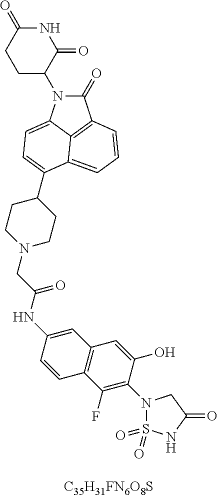 [SMILES: O=C1CCC(N2C(=O)c3cccc4c(C5CCN(CC(=O)Nc6ccc7c(F)c(N8CC(=O)NS8(=O)=O)c(O)cc7c6)CC5)ccc2c34)C(=O)N1 ; "C35H31FN6O8S"]

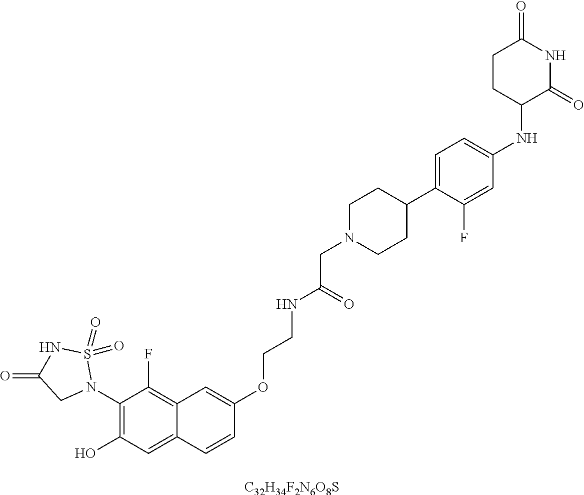 [SMILES: O=C(CN1CCC(c2ccc(NC3CCC(=O)NC3=O)cc2F)CC1)NCCOc1ccc2cc(O)c(N3CC(=O)NS3(=O)=O)c(F)c2c1 ; "C32H34F2N6O8S"]

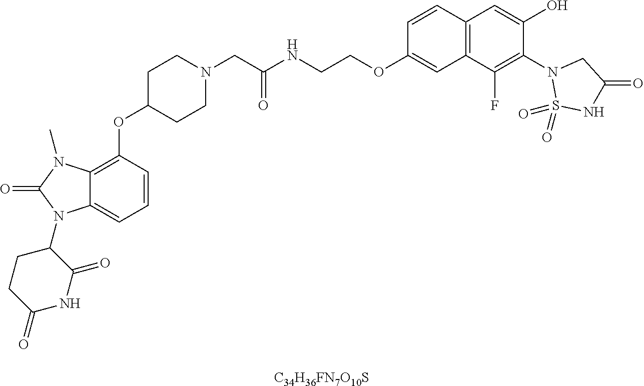 [SMILES: Cn1c(=O)n(C2CCC(=O)NC2=O)c2cccc(OC3CCN(CC(=O)NCCOc4ccc5cc(O)c(N6CC(=O)NS6(=O)=O)c(F)c5c4)CC3)c21 ; "C34H36FN7O10S"]

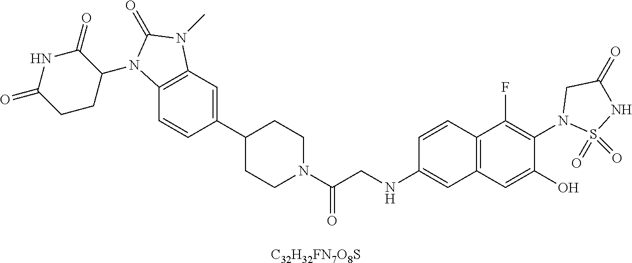 [SMILES: Cn1c(=O)n(C2CCC(=O)NC2=O)c2ccc(C3CCN(C(=O)CNc4ccc5c(F)c(N6CC(=O)NS6(=O)=O)c(O)cc5c4)CC3)cc21 ; "C32H32FN7O8S"]

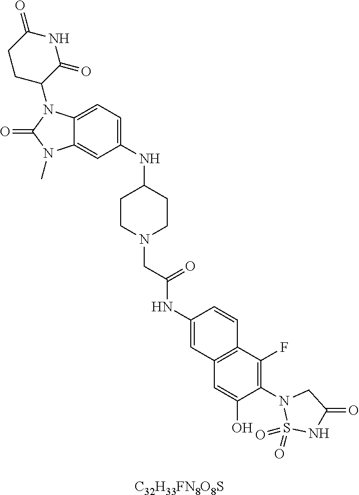 [SMILES: Cn1c(=O)n(C2CCC(=O)NC2=O)c2ccc(NC3CCN(CC(=O)Nc4ccc5c(F)c(N6CC(=O)NS6(=O)=O)c(O)cc5c4)CC3)cc21 ; "C32H33FN8O8S"]

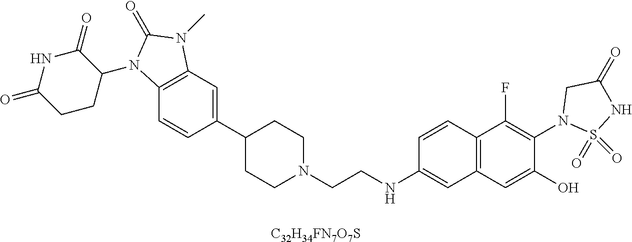

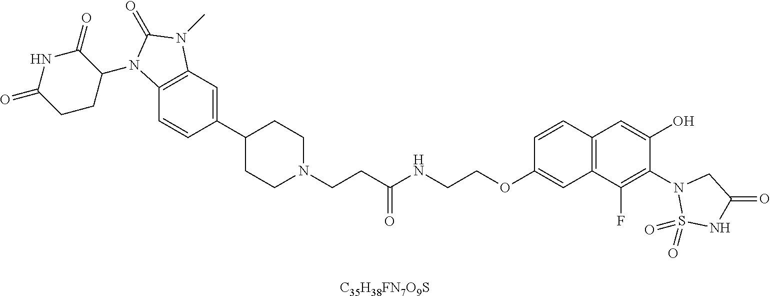 [SMILES: Cn1c(=O)n(C2CCC(=O)NC2=O)c2ccc(C3CCN(CCC(=O)NCCOc4ccc5cc(O)c(N6CC(=O)NS6(=O)=O)c(F)c5c4)CC3)cc21 ; "C35H38FN7O9S"]

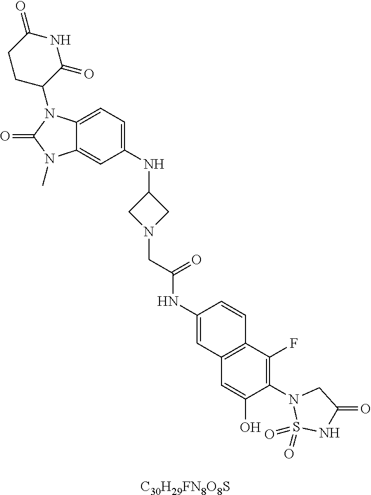 [SMILES: Cn1c(=O)n(C2CCC(=O)NC2=O)c2ccc(NC3CN(CC(=O)Nc4ccc5c(F)c(N6CC(=O)NS6(=O)=O)c(O)cc5c4)C3)cc21 ; "C30H29FN8O8S"]

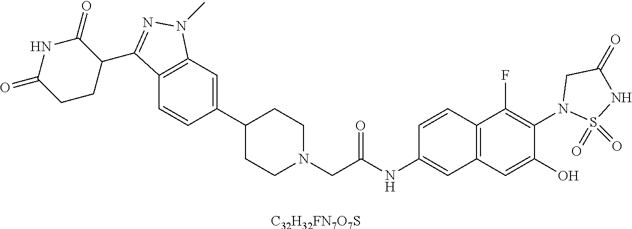

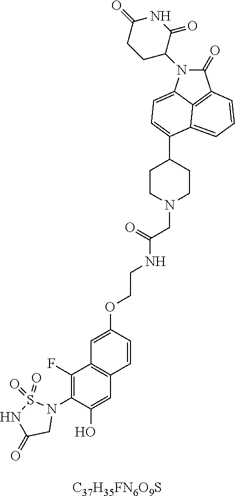 [SMILES: O=C(CN1CCC(c2ccc3c4c(cccc24)C(=O)N3C2CCC(=O)NC2=O)CC1)NCCOc1ccc2cc(O)c(N3CC(=O)NS3(=O)=O)c(F)c2c1 ; "C37H35FN6O9S"]

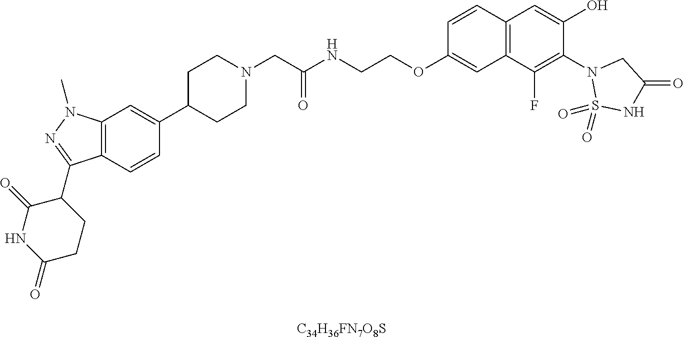 [SMILES: Cn1nc(C2CCC(=O)NC2=O)c2ccc(C3CCN(CC(=O)NCCOc4ccc5cc(O)c(N6CC(=O)NS6(=O)=O)c(F)c5c4)CC3)cc21 ; "C34H36FN7O8S"]

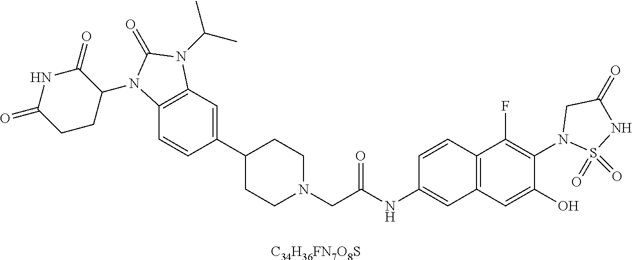

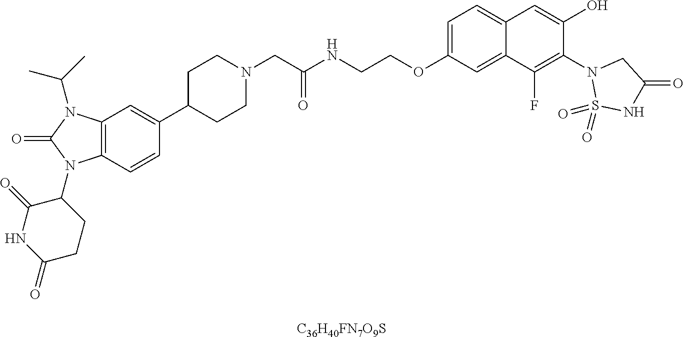 [SMILES: CC(C)n1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21 ; "C36H40FN7O9S"]

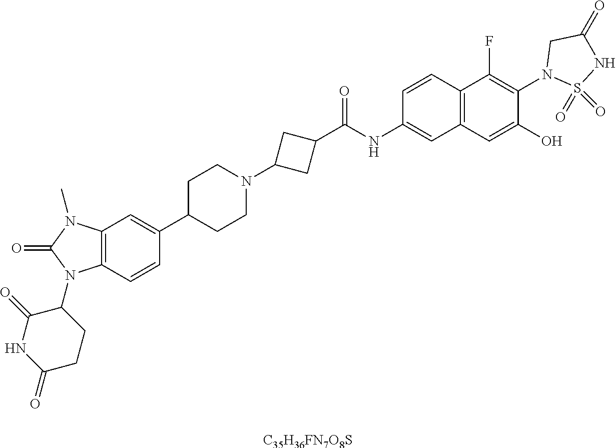 [SMILES: Cn1c(=O)n(C2CCC(=O)NC2=O)c2ccc(C3CCN(C4CC(C(=O)Nc5ccc6c(F)c(N7CC(=O)NS7(=O)=O)c(O)cc6c5)C4)CC3)cc21 ; "C35H36FN7O8S"]

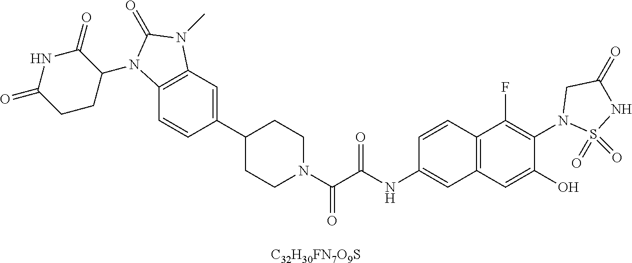 [SMILES: Cn1c(=O)n(C2CCC(=O)NC2=O)c2ccc(C3CCN(C(=O)C(=O)Nc4ccc5c(F)c(N6CC(=O)NS6(=O)=O)c(O)cc5c4)CC3)cc21 ; "C32H30FN7O9S"]

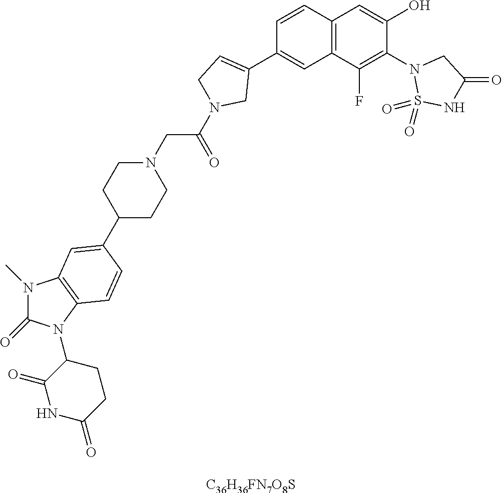 [SMILES: Cn1c(=O)n(C2CCC(=O)NC2=O)c2ccc(C3CCN(CC(=O)N4CC=C(c5ccc6cc(O)c(N7CC(=O)NS7(=O)=O)c(F)c6c5)C4)CC3)cc21 ; "C36H36FN7O8S"]

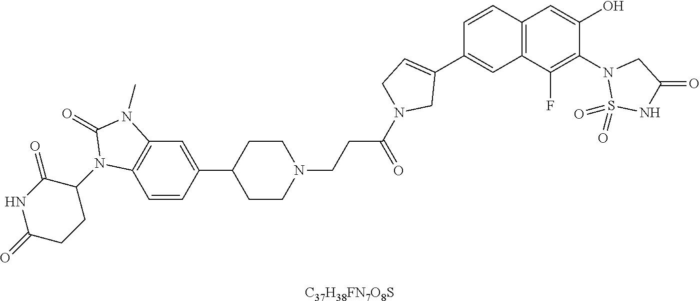 [SMILES: Cn1c(=O)n(C2CCC(=O)NC2=O)c2ccc(C3CCN(CCC(=O)N4CC=C(c5ccc6cc(O)c(N7CC(=O)NS7(=O)=O)c(F)c6c5)C4)CC3)cc21 ; "C37H38FN7O8S"]

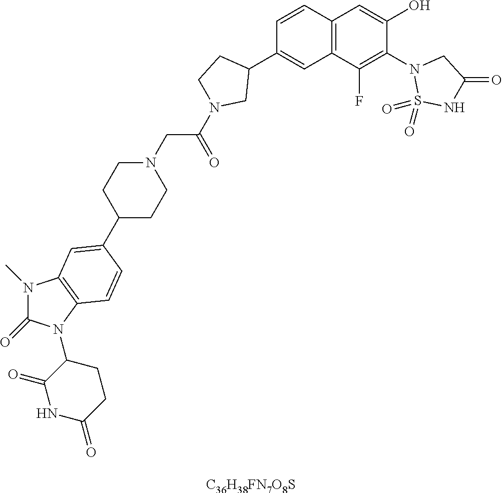 [SMILES: Cn1c(=O)n(C2CCC(=O)NC2=O)c2ccc(C3CCN(CC(=O)N4CCC(c5ccc6cc(O)c(N7CC(=O)NS7(=O)=O)c(F)c6c5)C4)CC3)cc21 ; "C36H38FN7O8S"]

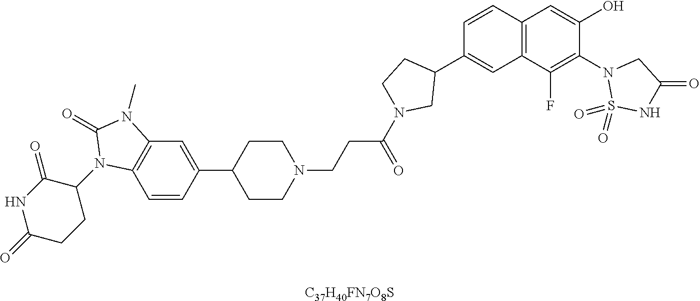 [SMILES: Cn1c(=O)n(C2CCC(=O)NC2=O)c2ccc(C3CCN(CCC(=O)N4CCC(c5ccc6cc(O)c(N7CC(=O)NS7(=O)=O)c(F)c6c5)C4)CC3)cc21 ; "C37H40FN7O8S"]

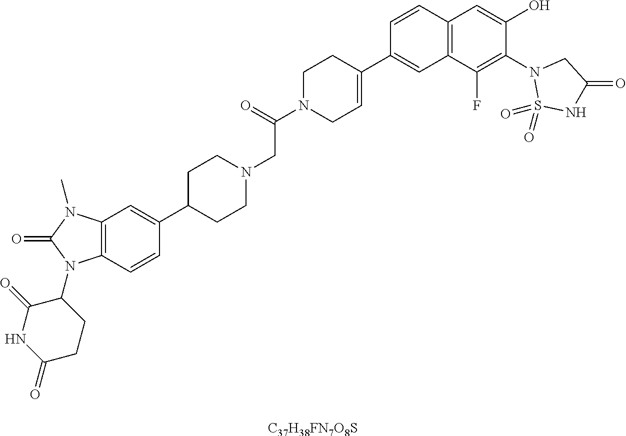 [SMILES: Cn1c(=O)n(C2CCC(=O)NC2=O)c2ccc(C3CCN(CC(=O)N4CC=C(c5ccc6cc(O)c(N7CC(=O)NS7(=O)=O)c(F)c6c5)CC4)CC3)cc21 ; "C37H38FN7O8S"]

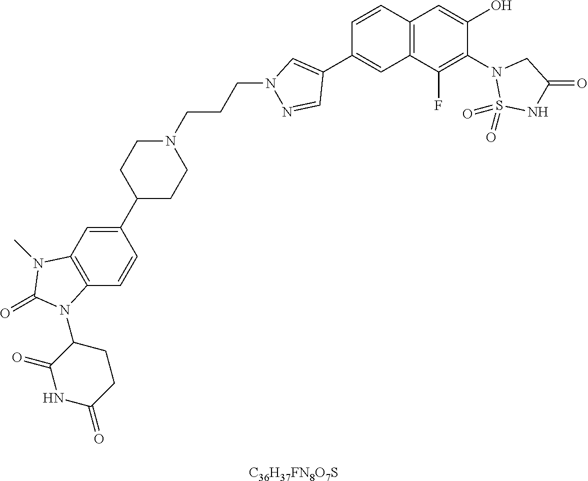 [SMILES: Cn1c(=O)n(C2CCC(=O)NC2=O)c2ccc(C3CCN(CCCn4cc(-c5ccc6cc(O)c(N7CC(=O)NS7(=O)=O)c(F)c6c5)cn4)CC3)cc21 ; "C36H37FN8O7S"]

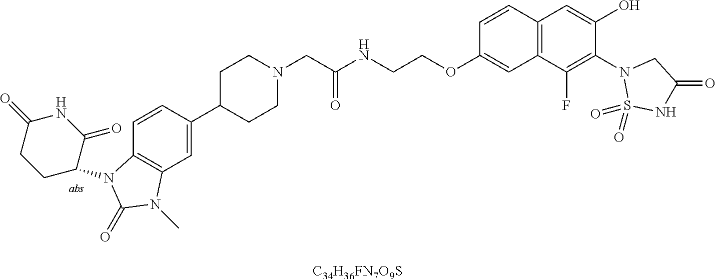 [SMILES: Cn1c(=O)n([C@@H]2CCC(=O)NC2=O)c2ccc(C3CCN(CC(=O)NCCOc4ccc5cc(O)c(N6CC(=O)NS6(=O)=O)c(F)c5c4)CC3)cc21 ; "C34H36FN7O9S"]

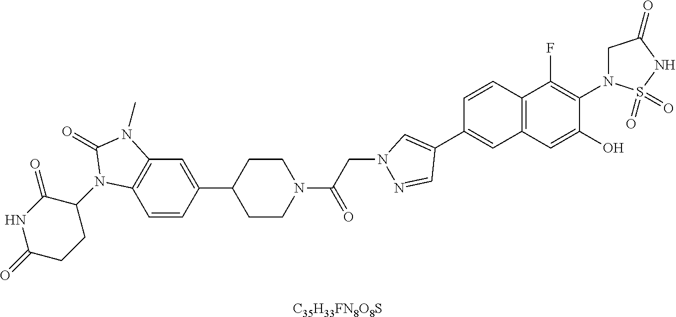 [SMILES: Cn1c(=O)n(C2CCC(=O)NC2=O)c2ccc(C3CCN(C(=O)Cn4cc(-c5ccc6c(F)c(N7CC(=O)NS7(=O)=O)c(O)cc6c5)cn4)CC3)cc21 ; "C35H33FN8O8S"]

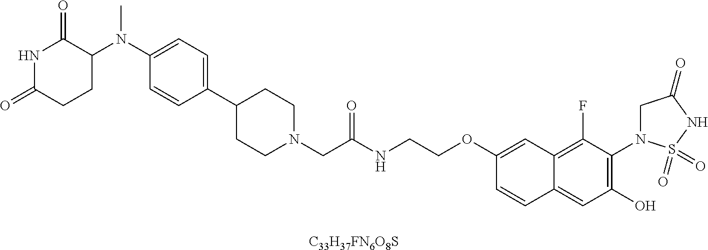 [SMILES: CN(c1ccc(C2CCN(CC(=O)NCCOc3ccc4cc(O)c(N5CC(=O)NS5(=O)=O)c(F)c4c3)CC2)cc1)C1CCC(=O)NC1=O ; "C33H37FN6O8S"]

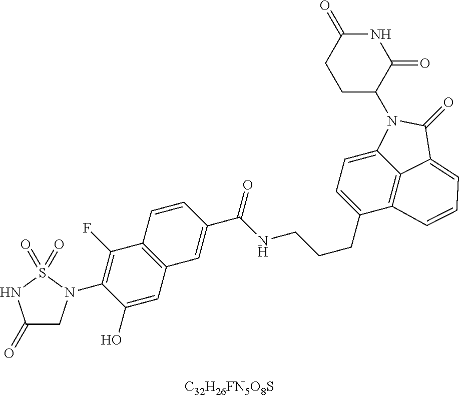 [SMILES: O=C1CCC(N2C(=O)c3cccc4c(CCCNC(=O)c5ccc6c(F)c(N7CC(=O)NS7(=O)=O)c(O)cc6c5)ccc2c34)C(=O)N1 ; "C32H26FN5O8S"]

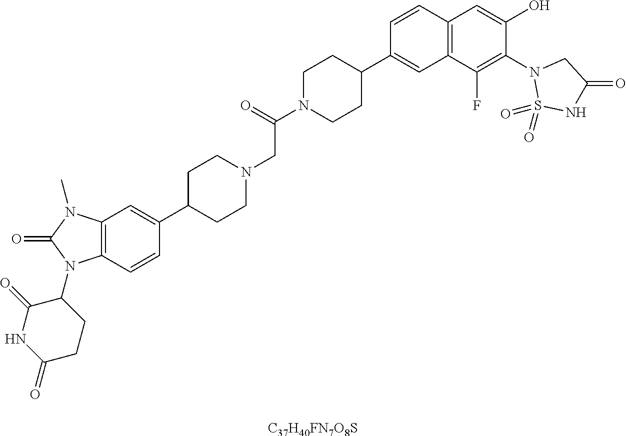 [SMILES: Cn1c(=O)n(C2CCC(=O)NC2=O)c2ccc(C3CCN(CC(=O)N4CCC(c5ccc6cc(O)c(N7CC(=O)NS7(=O)=O)c(F)c6c5)CC4)CC3)cc21 ; "C37H40FN7O8S"]

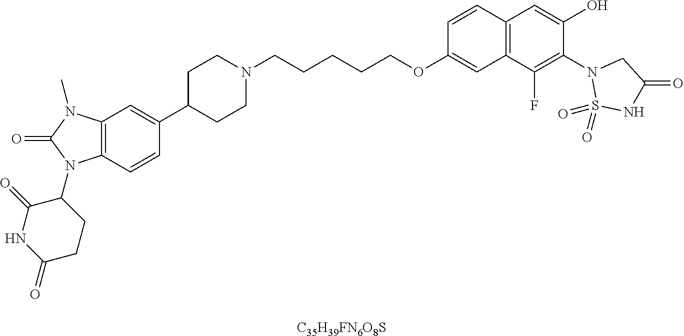 [SMILES: Cn1c(=O)n(C2CCC(=O)NC2=O)c2ccc(C3CCN(CCCCCOc4ccc5cc(O)c(N6CC(=O)NS6(=O)=O)c(F)c5c4)CC3)cc21 ; "C35H39FN6O8S"]

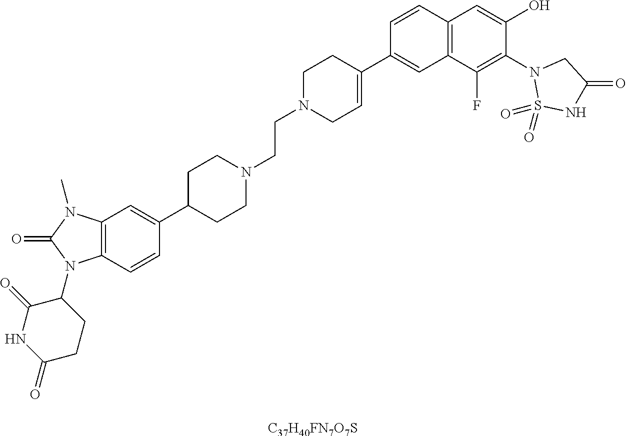 [SMILES: Cn1c(=O)n(C2CCC(=O)NC2=O)c2ccc(C3CCN(CCN4CC=C(c5ccc6cc(O)c(N7CC(=O)NS7(=O)=O)c(F)c6c5)CC4)CC3)cc21 ; "C37H40FN7O7S"]

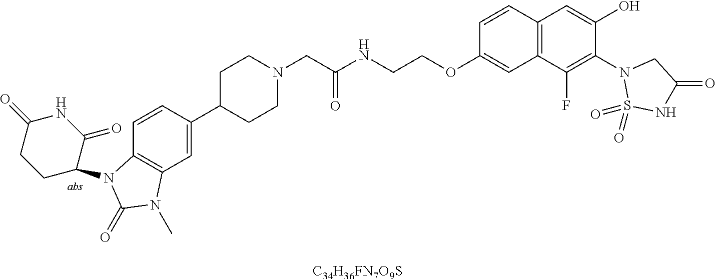 [SMILES: Cn1c(=O)n([C@H]2CCC(=O)NC2=O)c2ccc(C3CCN(CC(=O)NCCOc4ccc5cc(O)c(N6CC(=O)NS6(=O)=O)c(F)c5c4)CC3)cc21 ; "C34H36FN7O9S"]

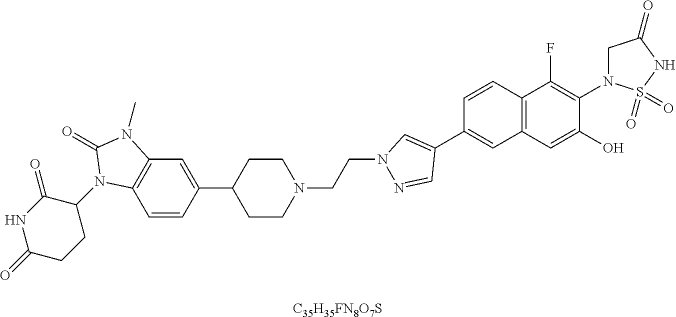 [SMILES: Cn1c(=O)n(C2CCC(=O)NC2=O)c2ccc(C3CCN(CCn4cc(-c5ccc6c(F)c(N7CC(=O)NS7(=O)=O)c(O)cc6c5)cn4)CC3)cc21 ; "C35H35FN8O7S"]

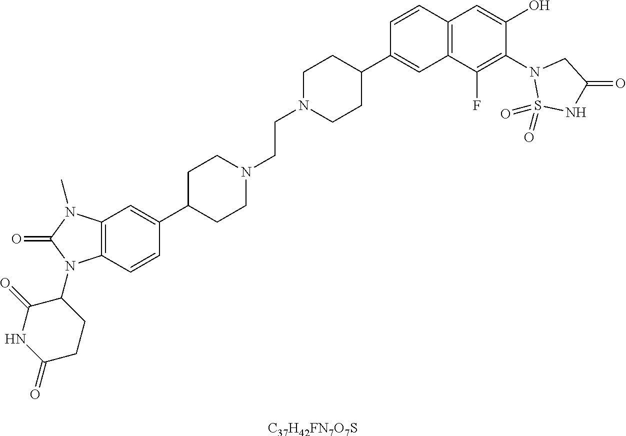 [SMILES: Cn1c(=O)n(C2CCC(=O)NC2=O)c2ccc(C3CCN(CCN4CCC(c5ccc6cc(O)c(N7CC(=O)NS7(=O)=O)c(F)c6c5)CC4)CC3)cc21 ; "C37H42FN7O7S"]

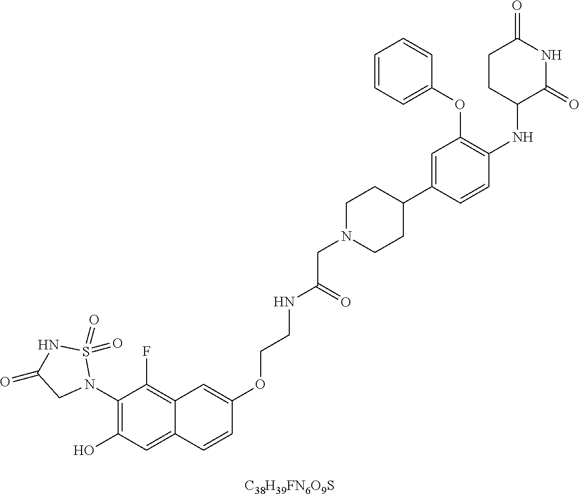 [SMILES: O=C(CN1CCC(c2ccc(NC3CCC(=O)NC3=O)c(Oc3ccccc3)c2)CC1)NCCOc1ccc2cc(O)c(N3CC(=O)NS3(=O)=O)c(F)c2c1 ; "C38H39FN6O9S"]

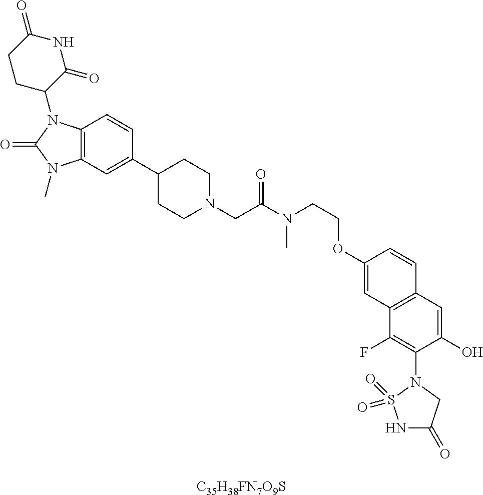 [SMILES: CN(CCOc1ccc2cc(O)c(N3CC(=O)NS3(=O)=O)c(F)c2c1)C(=O)CN1CCC(c2ccc3c(c2)n(C)c(=O)n3C2CCC(=O)NC2=O)CC1 ; "C35H38FN7O9S"]

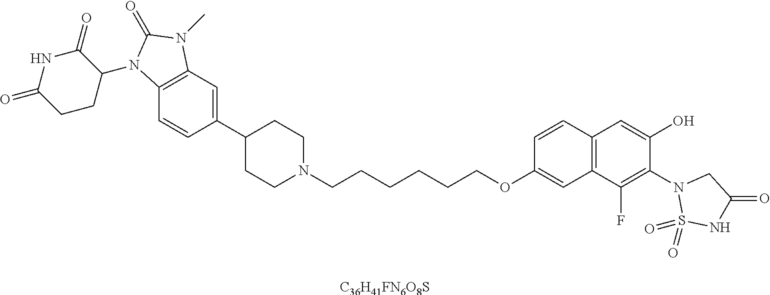 [SMILES: Cn1c(=O)n(C2CCC(=O)NC2=O)c2ccc(C3CCN(CCCCCCOc4ccc5cc(O)c(N6CC(=O)NS6(=O)=O)c(F)c5c4)CC3)cc21 ; "C36H41FN6O8S"]

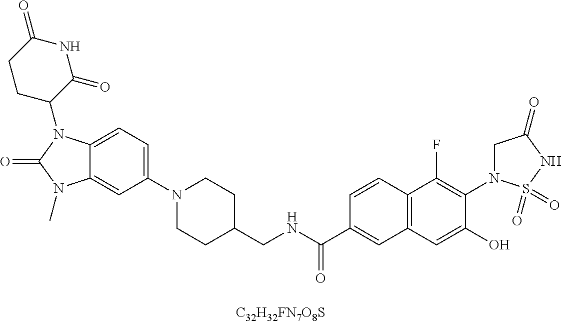 [SMILES: Cn1c(=O)n(C2CCC(=O)NC2=O)c2ccc(N3CCC(CNC(=O)c4ccc5c(F)c(N6CC(=O)NS6(=O)=O)c(O)cc5c4)CC3)cc21 ; "C32H32FN7O8S"]

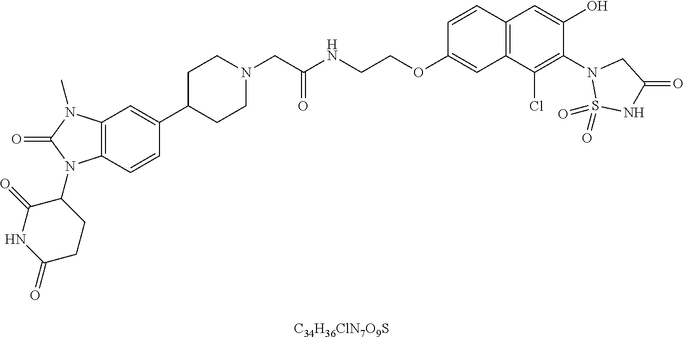 [SMILES: Cn1c(=O)n(C2CCC(=O)NC2=O)c2ccc(C3CCN(CC(=O)NCCOc4ccc5cc(O)c(N6CC(=O)NS6(=O)=O)c(Cl)c5c4)CC3)cc21 ; "C34H36ClN7O9S"]

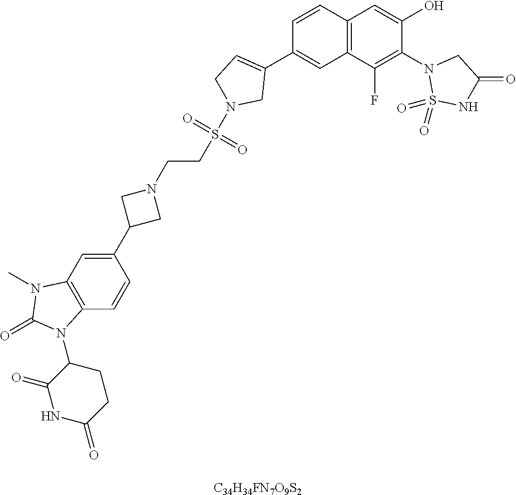 [SMILES: Cn1c(=O)n(C2CCC(=O)NC2=O)c2ccc(C3CN(CCS(=O)(=O)N4CC=C(c5ccc6cc(O)c(N7CC(=O)NS7(=O)=O)c(F)c6c5)C4)C3)cc21 ; "C34H34FN7O9S2"]

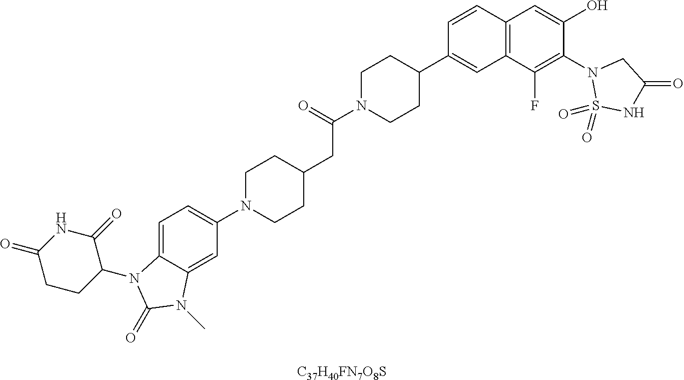 [SMILES: Cn1c(=O)n(C2CCC(=O)NC2=O)c2ccc(N3CCC(CC(=O)N4CCC(c5ccc6cc(O)c(N7CC(=O)NS7(=O)=O)c(F)c6c5)CC4)CC3)cc21 ; "C37H40FN7O8S"]

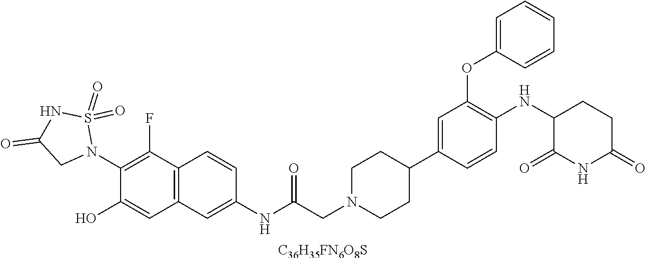

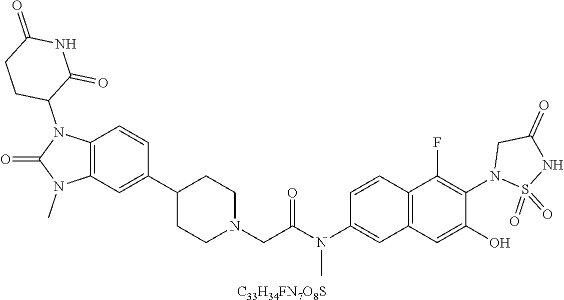 [SMILES: CN(C(=O)CN1CCC(c2ccc3c(c2)n(C)c(=O)n3C2CCC(=O)NC2=O)CC1)c1ccc2c(F)c(N3CC(=O)NS3(=O)=O)c(O)cc2c1 ; "C33H34FN7O8S"]

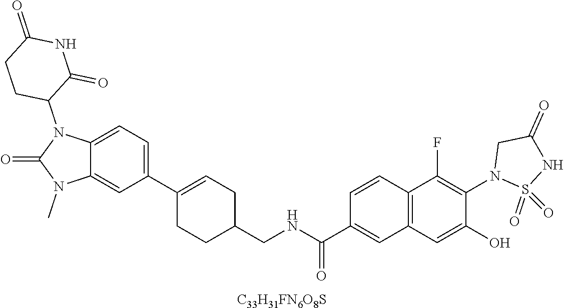 [SMILES: Cn1c(=O)n(C2CCC(=O)NC2=O)c2ccc(C3=CCC(CNC(=O)c4ccc5c(F)c(N6CC(=O)NS6(=O)=O)c(O)cc5c4)CC3)cc21 ; "C33H31FN6O8S"]

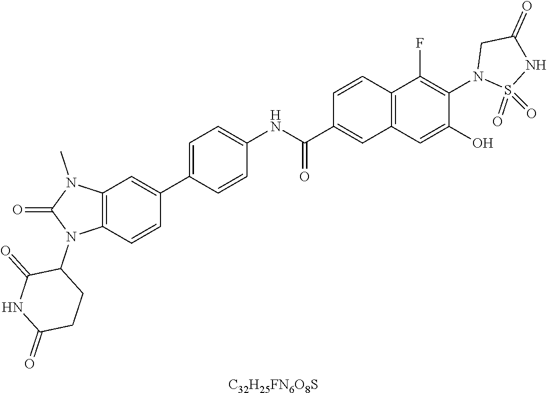 [SMILES: Cn1c(=O)n(C2CCC(=O)NC2=O)c2ccc(-c3ccc(NC(=O)c4ccc5c(F)c(N6CC(=O)NS6(=O)=O)c(O)cc5c4)cc3)cc21 ; "C32H25FN6O8S"]

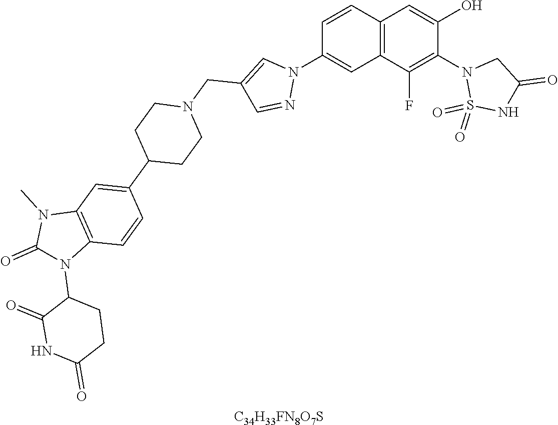 [SMILES: Cn1c(=O)n(C2CCC(=O)NC2=O)c2ccc(C3CCN(Cc4cnn(-c5ccc6cc(O)c(N7CC(=O)NS7(=O)=O)c(F)c6c5)c4)CC3)cc21 ; "C34H33FN8O7S"]

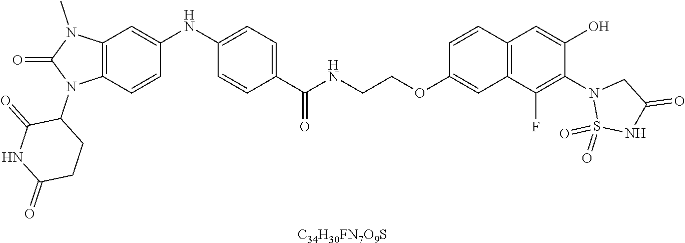 [SMILES: Cn1c(=O)n(C2CCC(=O)NC2=O)c2ccc(Nc3ccc(C(=O)NCCOc4ccc5cc(O)c(N6CC(=O)NS6(=O)=O)c(F)c5c4)cc3)cc21 ; "C34H30FN7O9S"]

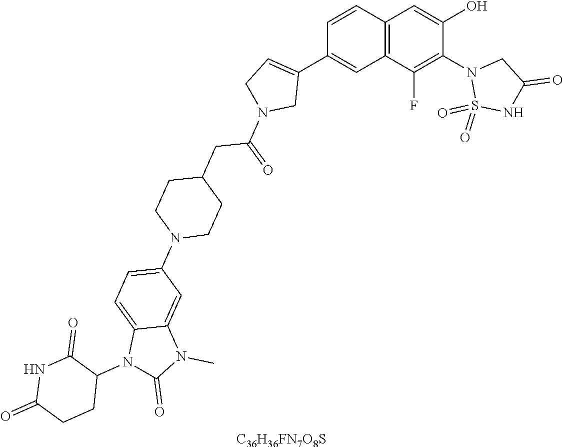 [SMILES: Cn1c(=O)n(C2CCC(=O)NC2=O)c2ccc(N3CCC(CC(=O)N4CC=C(c5ccc6cc(O)c(N7CC(=O)NS7(=O)=O)c(F)c6c5)C4)CC3)cc21 ; "C36H36FN7O8S"]

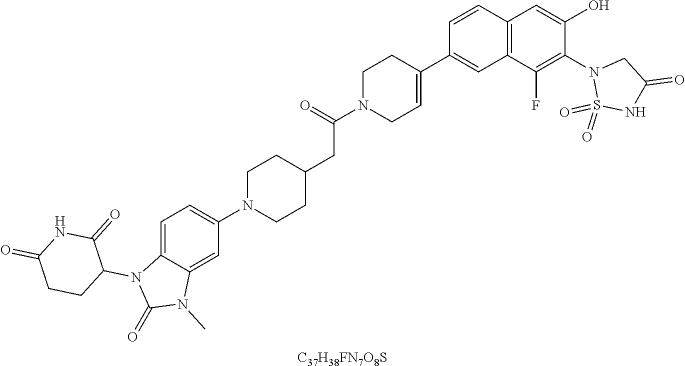 [SMILES: Cn1c(=O)n(C2CCC(=O)NC2=O)c2ccc(N3CCC(CC(=O)N4CC=C(c5ccc6cc(O)c(N7CC(=O)NS7(=O)=O)c(F)c6c5)CC4)CC3)cc21 ; "C37H38FN7O8S"]

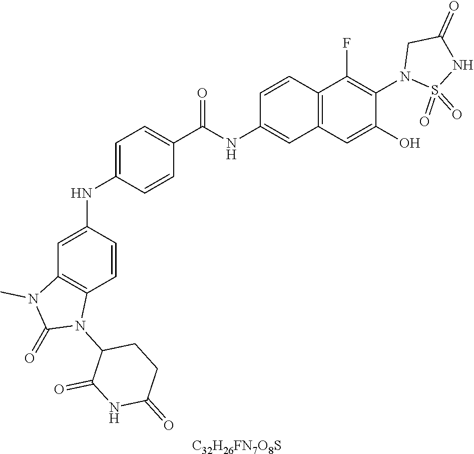

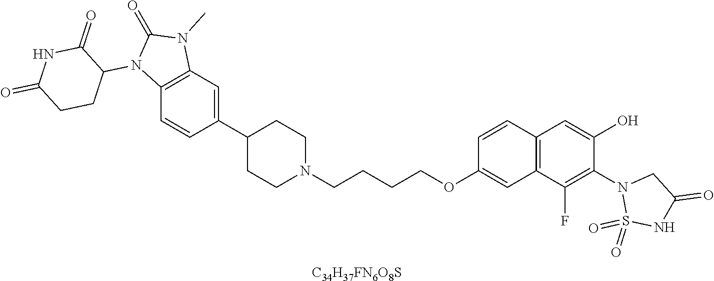 [SMILES: Cn1c(=O)n(C2CCC(=O)NC2=O)c2ccc(C3CCN(CCCCOc4ccc5cc(O)c(N6CC(=O)NS6(=O)=O)c(F)c5c4)CC3)cc21 ; "C34H37FN6O8S"]

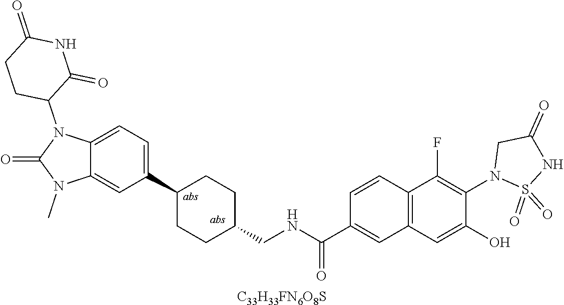 [SMILES: Cn1c(=O)n(C2CCC(=O)NC2=O)c2ccc([C@H]3CC[C@H](CNC(=O)c4ccc5c(F)c(N6CC(=O)NS6(=O)=O)c(O)cc5c4)CC3)cc21 ; "C33H33FN6O8S"]

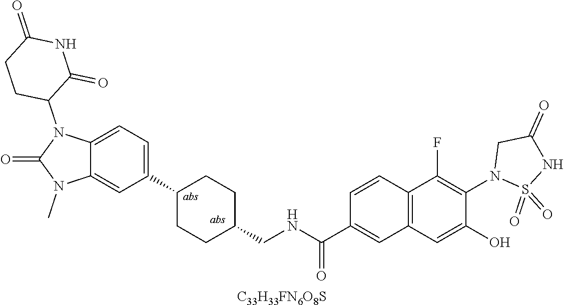 [SMILES: Cn1c(=O)n(C2CCC(=O)NC2=O)c2ccc([C@H]3CC[C@@H](CNC(=O)c4ccc5c(F)c(N6CC(=O)NS6(=O)=O)c(O)cc5c4)CC3)cc21 ; "C33H33FN6O8S"]

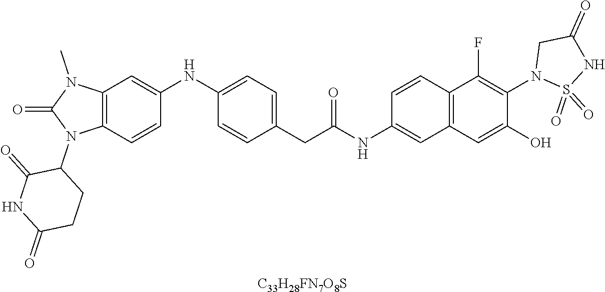 [SMILES: Cn1c(=O)n(C2CCC(=O)NC2=O)c2ccc(Nc3ccc(CC(=O)Nc4ccc5c(F)c(N6CC(=O)NS6(=O)=O)c(O)cc5c4)cc3)cc21 ; "C33H28FN7O8S"]

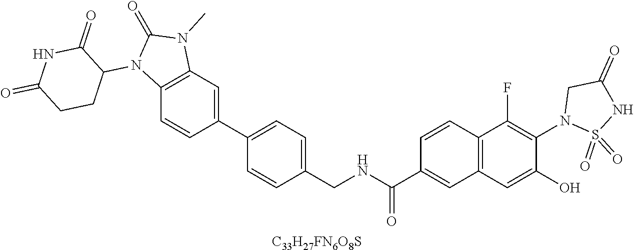 [SMILES: Cn1c(=O)n(C2CCC(=O)NC2=O)c2ccc(-c3ccc(CNC(=O)c4ccc5c(F)c(N6CC(=O)NS6(=O)=O)c(O)cc5c4)cc3)cc21 ; "C33H27FN6O8S"]

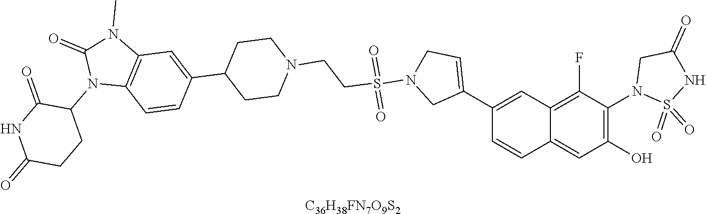 [SMILES: Cn1c(=O)n(C2CCC(=O)NC2=O)c2ccc(C3CCN(CCS(=O)(=O)N4CC=C(c5ccc6cc(O)c(N7CC(=O)NS7(=O)=O)c(F)c6c5)C4)CC3)cc21 ; "C36H38FN7O9S2"]

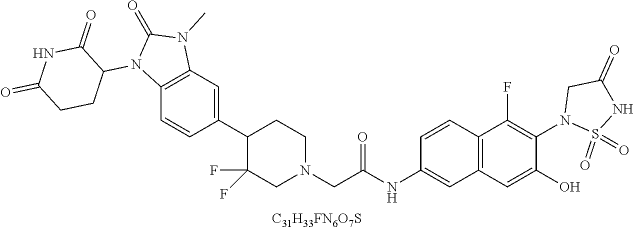

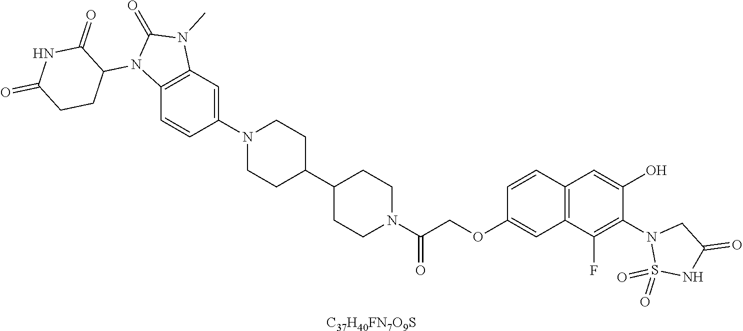 [SMILES: Cn1c(=O)n(C2CCC(=O)NC2=O)c2ccc(N3CCC(C4CCN(C(=O)COc5ccc6cc(O)c(N7CC(=O)NS7(=O)=O)c(F)c6c5)CC4)CC3)cc21 ; "C37H40FN7O9S"]

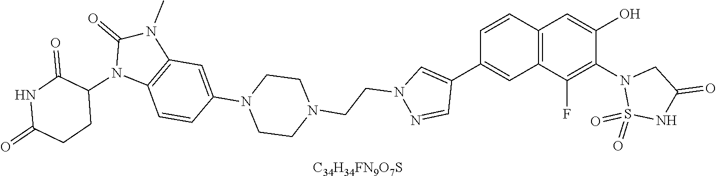

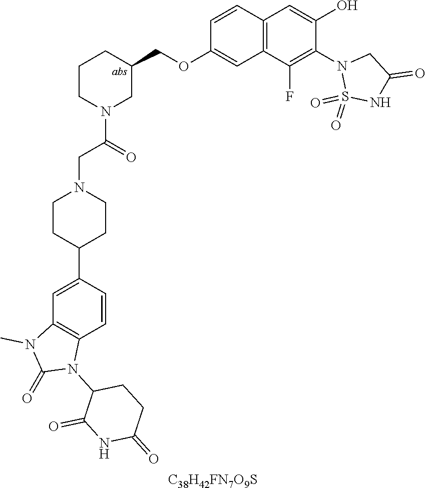

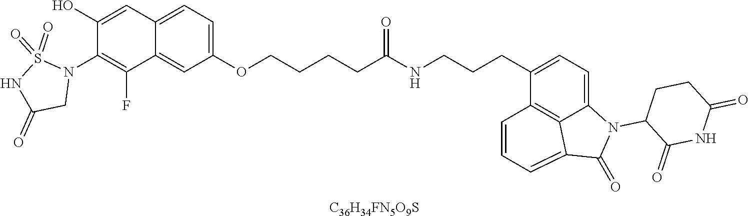 [SMILES: O=C(CCCCOc1ccc2cc(O)c(N3CC(=O)NS3(=O)=O)c(F)c2c1)NCCCc1ccc2c3c(cccc13)C(=O)N2C1CCC(=O)NC1=O ; "C36H34FN5O9S"]

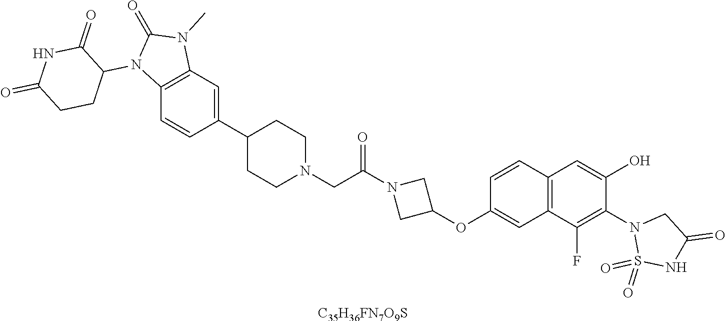 [SMILES: Cn1c(=O)n(C2CCC(=O)NC2=O)c2ccc(C3CCN(CC(=O)N4CC(Oc5ccc6cc(O)c(N7CC(=O)NS7(=O)=O)c(F)c6c5)C4)CC3)cc21 ; "C35H36FN7O9S"]

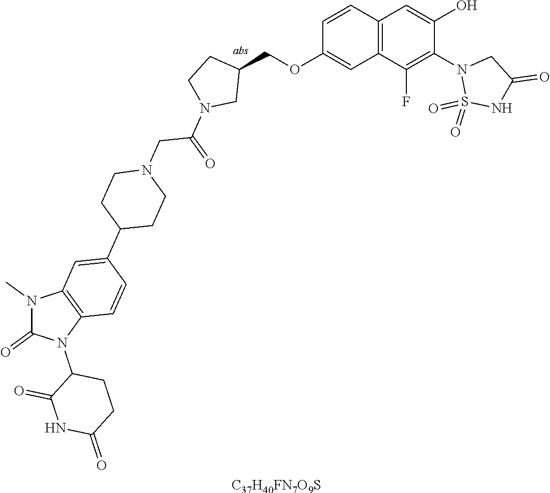 [SMILES: Cn1c(=O)n(C2CCC(=O)NC2=O)c2ccc(C3CCN(CC(=O)N4CC[C@@H](COc5ccc6cc(O)c(N7CC(=O)NS7(=O)=O)c(F)c6c5)C4)CC3)cc21 ; "C37H40FN7O9S"]

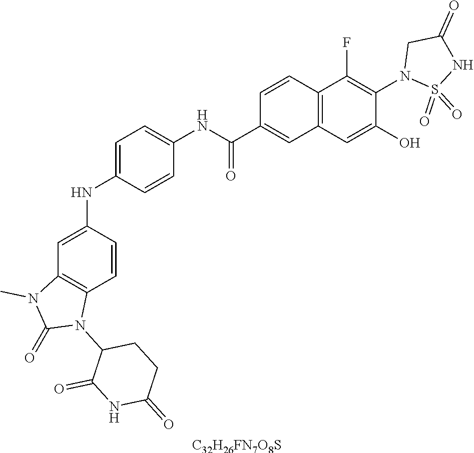

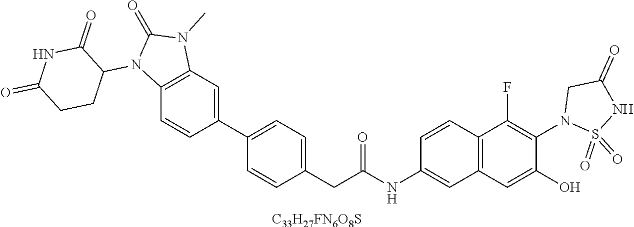

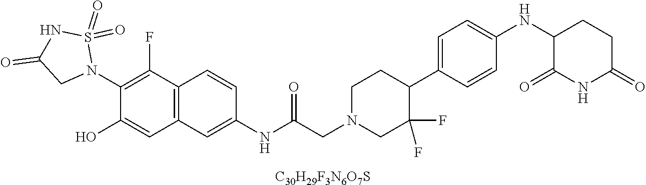

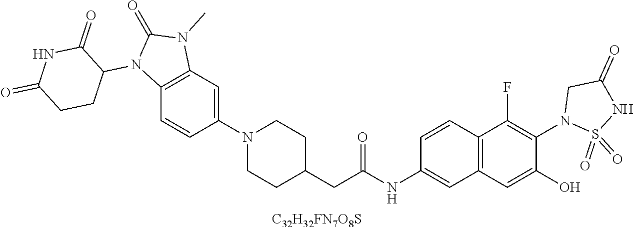

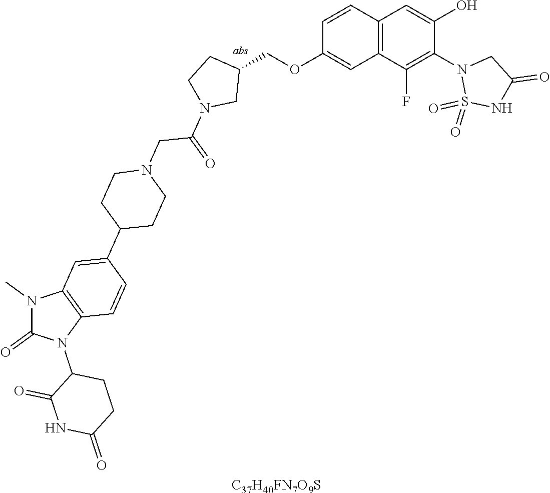 [SMILES: Cn1c(=O)n(C2CCC(=O)NC2=O)c2ccc(C3CCN(CC(=O)N4CC[C@H](COc5ccc6cc(O)c(N7CC(=O)NS7(=O)=O)c(F)c6c5)C4)CC3)cc21 ; "C37H40FN7O9S"]

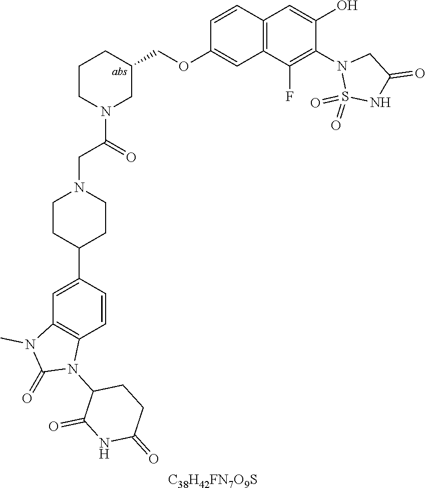

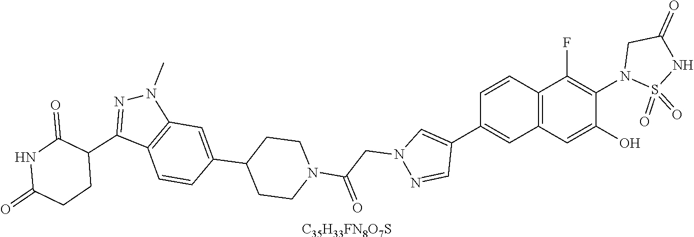

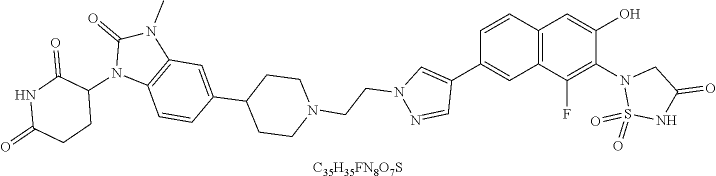

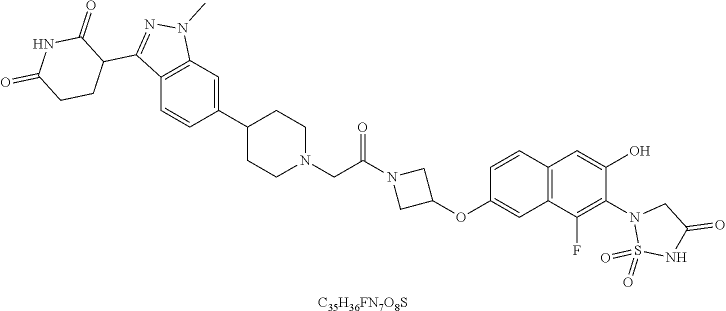 [SMILES: Cn1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3)cc21 ; "C35H36FN7O8S"]

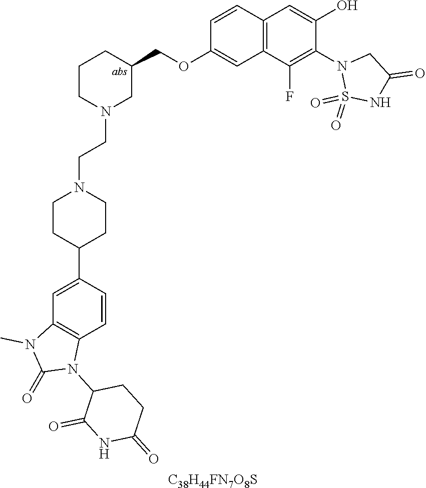

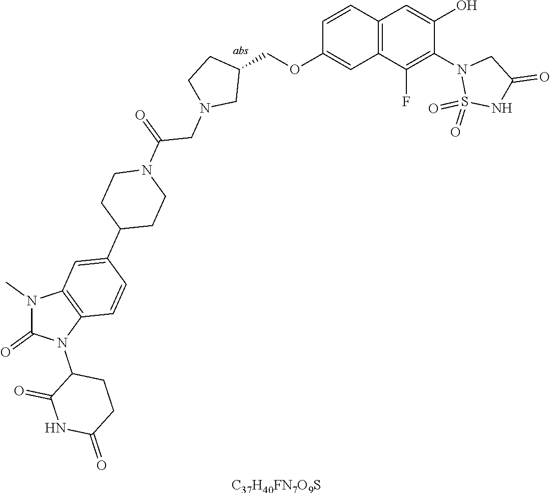 [SMILES: Cn1c(=O)n(C2CCC(=O)NC2=O)c2ccc(C3CCN(C(=O)CN4CC[C@H](COc5ccc6cc(O)c(N7CC(=O)NS7(=O)=O)c(F)c6c5)C4)CC3)cc21 ; "C37H40FN7O9S"]

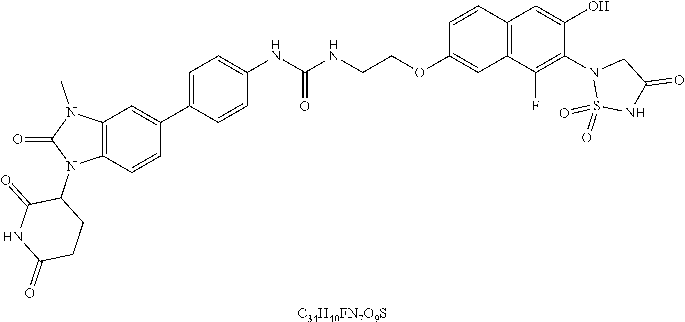 [SMILES: Cn1c(=O)n(C2CCC(=O)NC2=O)c2ccc(-c3ccc(NC(=O)NCCOc4ccc5cc(O)c(N6CC(=O)NS6(=O)=O)c(F)c5c4)cc3)cc21 ; "C34H40FN7O9S"]

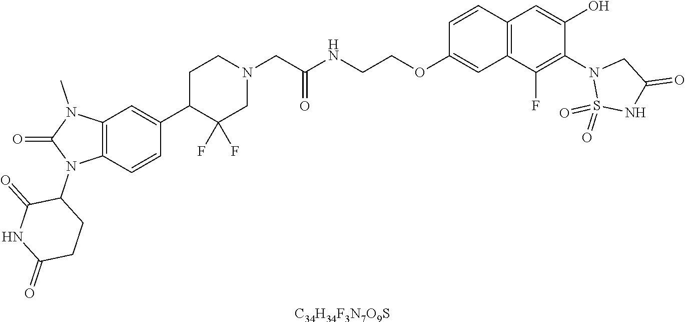 [SMILES: Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3(F)F)cc21 ; "C34H34F3N7O9S"]

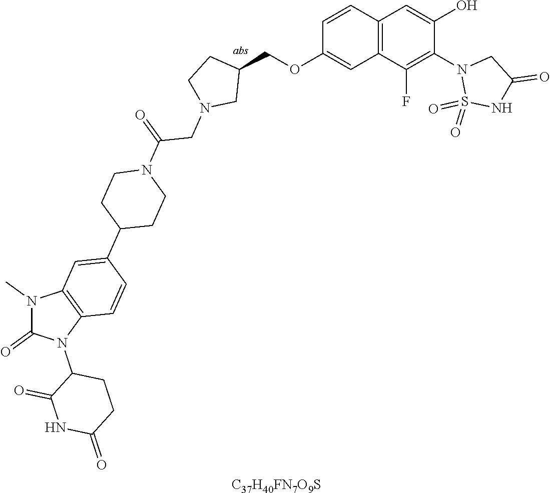 [SMILES: Cn1c(=O)n(C2CCC(=O)NC2=O)c2ccc(C3CCN(C(=O)CN4CC[C@@H](COc5ccc6cc(O)c(N7CC(=O)NS7(=O)=O)c(F)c6c5)C4)CC3)cc21 ; "C37H40FN7O9S"]

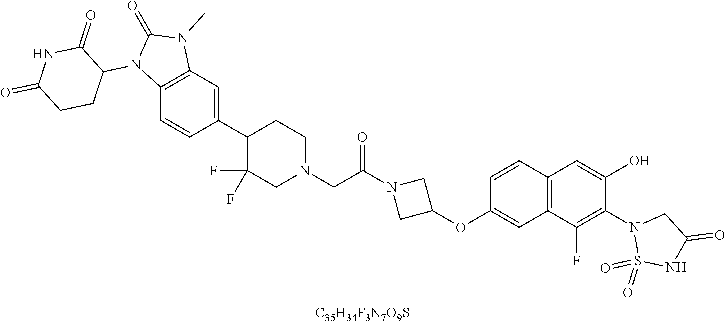 [SMILES: Cn1c(=O)n(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21 ; "C35H34F3N7O9S"]

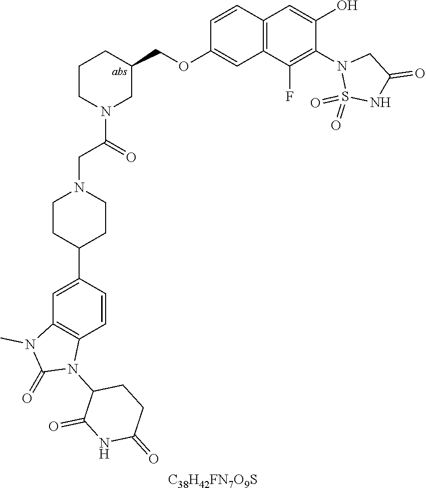

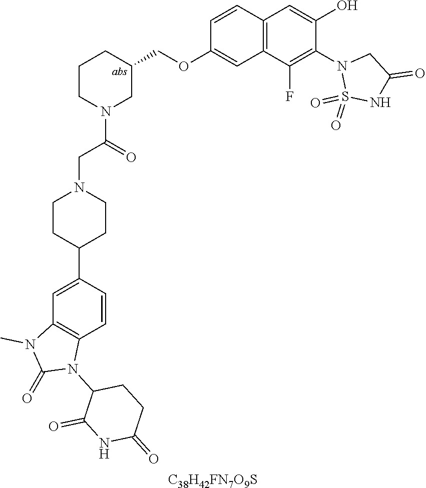

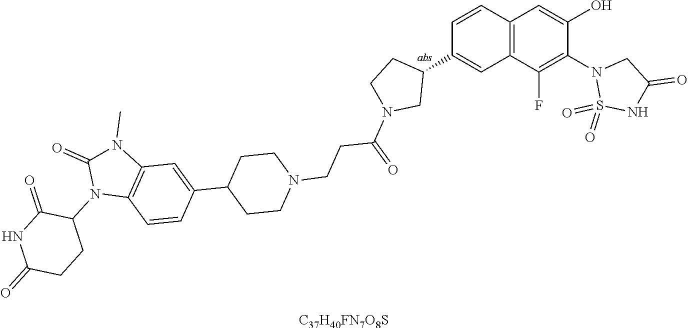 [SMILES: Cn1c(=O)n(C2CCC(=O)NC2=O)c2ccc(C3CCN(CCC(=O)N4CC[C@H](c5ccc6cc(O)c(N7CC(=O)NS7(=O)=O)c(F)c6c5)C4)CC3)cc21 ; "C37H40FN7O8S"]

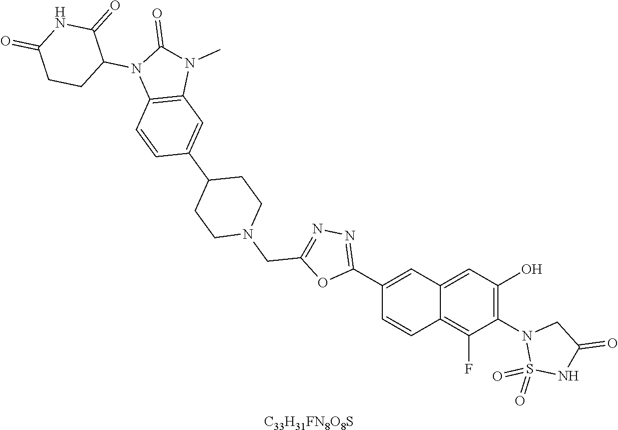 [SMILES: Cn1c(=O)n(C2CCC(=O)NC2=O)c2ccc(C3CCN(Cc4nnc(-c5ccc6c(F)c(N7CC(=O)NS7(=O)=O)c(O)cc6c5)o4)CC3)cc21 ; "C33H31FN8O8S"]

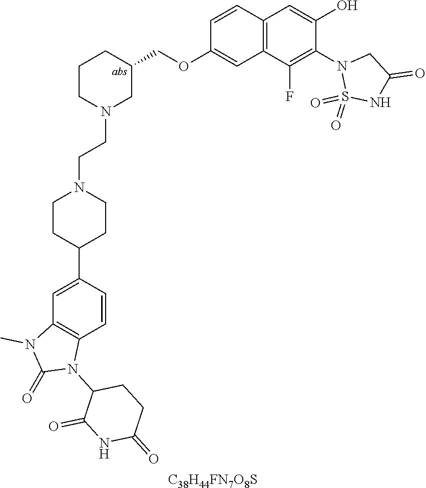

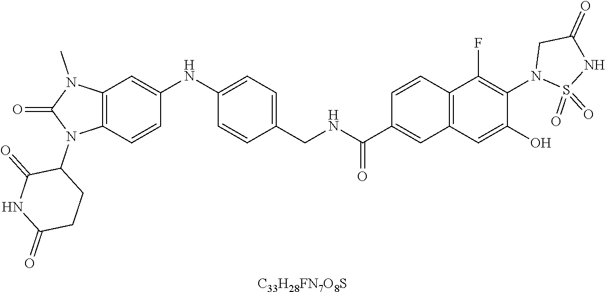 [SMILES: Cn1c(=O)n(C2CCC(=O)NC2=O)c2ccc(Nc3ccc(CNC(=O)c4ccc5c(F)c(N6CC(=O)NS6(=O)=O)c(O)cc5c4)cc3)cc21 ; "C33H28FN7O8S"]

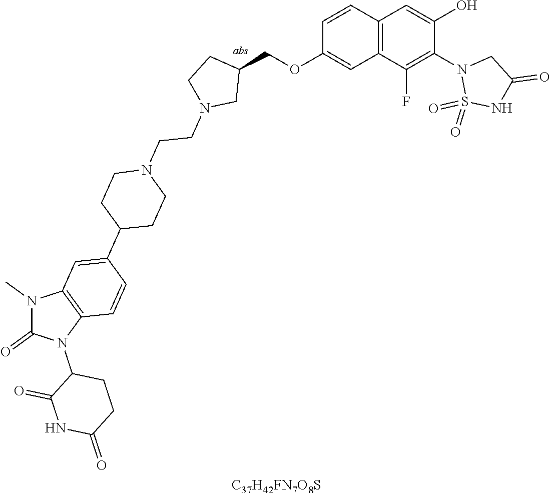 [SMILES: Cn1c(=O)n(C2CCC(=O)NC2=O)c2ccc(C3CCN(CCN4CC[C@@H](COc5ccc6cc(O)c(N7CC(=O)NS7(=O)=O)c(F)c6c5)C4)CC3)cc21 ; "C37H42FN7O8S"]

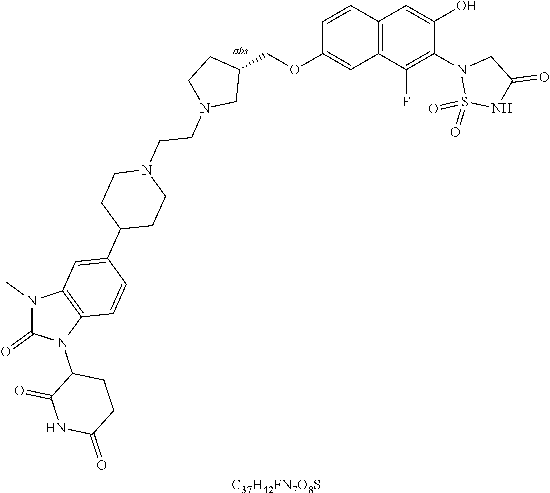 [SMILES: Cn1c(=O)n(C2CCC(=O)NC2=O)c2ccc(C3CCN(CCN4CC[C@H](COc5ccc6cc(O)c(N7CC(=O)NS7(=O)=O)c(F)c6c5)C4)CC3)cc21 ; "C37H42FN7O8S"]

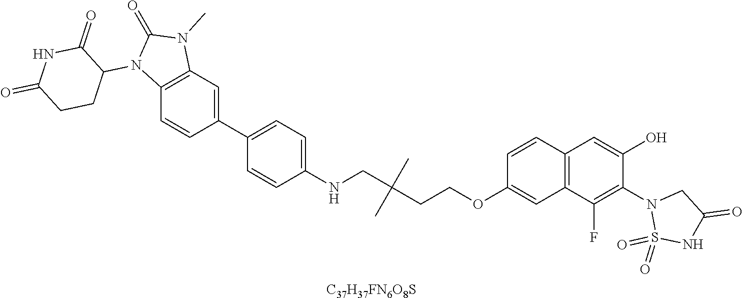 [SMILES: Cn1c(=O)n(C2CCC(=O)NC2=O)c2ccc(-c3ccc(NCC(C)(C)CCOc4ccc5cc(O)c(N6CC(=O)NS6(=O)=O)c(F)c5c4)cc3)cc21 ; "C37H37FN6O8S"]

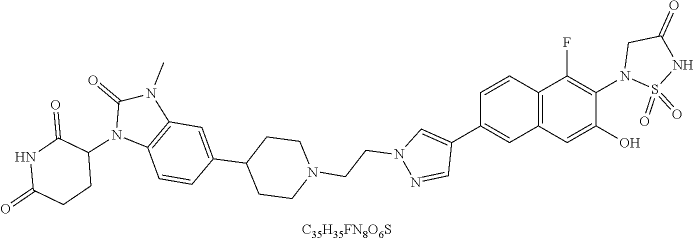

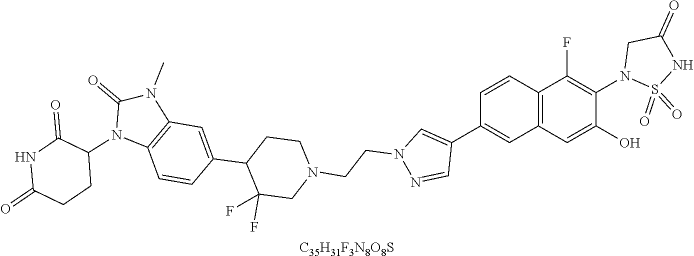 [SMILES: Cn1c(=O)n(C2CCC(=O)NC2=O)c2ccc(C3CCN(CCn4cc(-c5ccc6c(F)c(N7CC(=O)NS7(=O)=O)c(O)cc6c5)cn4)CC3(F)F)cc21 ; "C35H31F3N8O8S"]

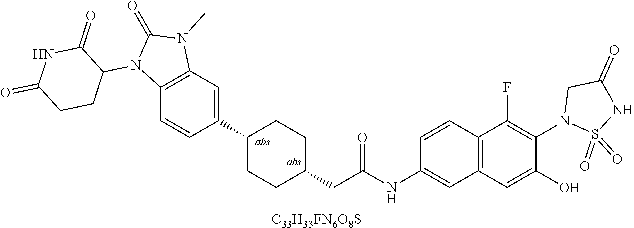

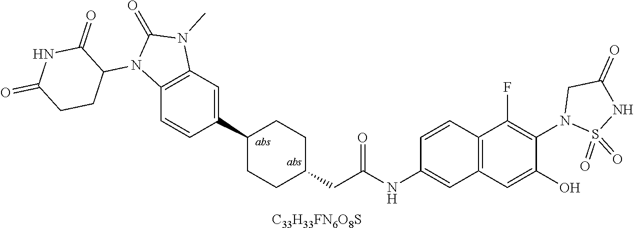

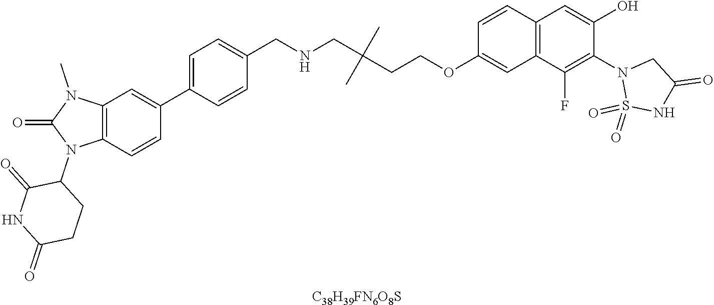 [SMILES: Cn1c(=O)n(C2CCC(=O)NC2=O)c2ccc(-c3ccc(CNCC(C)(C)CCOc4ccc5cc(O)c(N6CC(=O)NS6(=O)=O)c(F)c5c4)cc3)cc21 ; "C38H39FN6O8S"]

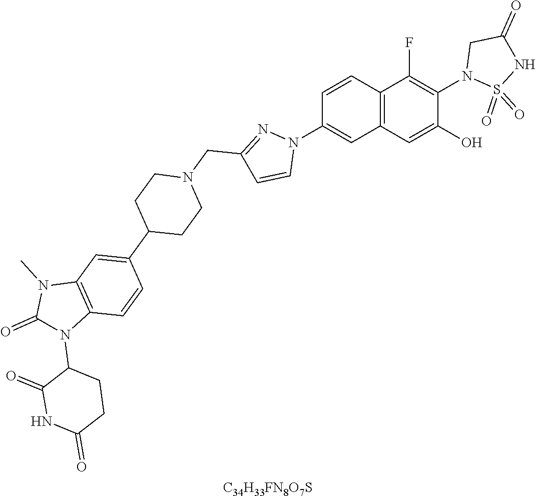 [SMILES: Cn1c(=O)n(C2CCC(=O)NC2=O)c2ccc(C3CCN(Cc4ccn(-c5ccc6c(F)c(N7CC(=O)NS7(=O)=O)c(O)cc6c5)n4)CC3)cc21 ; "C34H33FN8O7S"]

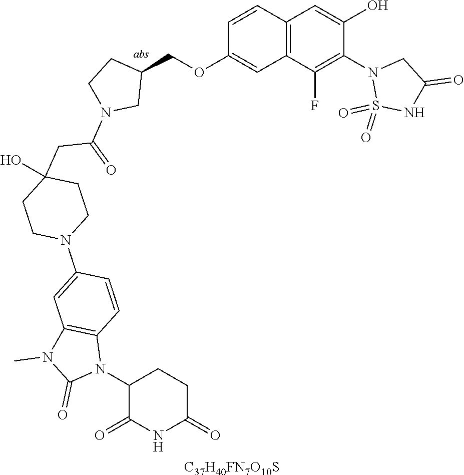

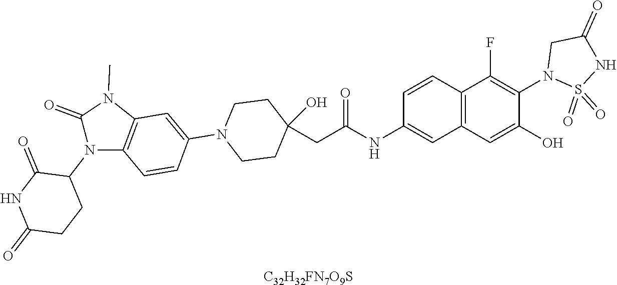 [SMILES: Cn1c(=O)n(C2CCC(=O)NC2=O)c2ccc(N3CCC(O)(CC(=O)Nc4ccc5c(F)c(N6CC(=O)NS6(=O)=O)c(O)cc5c4)CC3)cc21 ; "C32H32FN7O9S"]

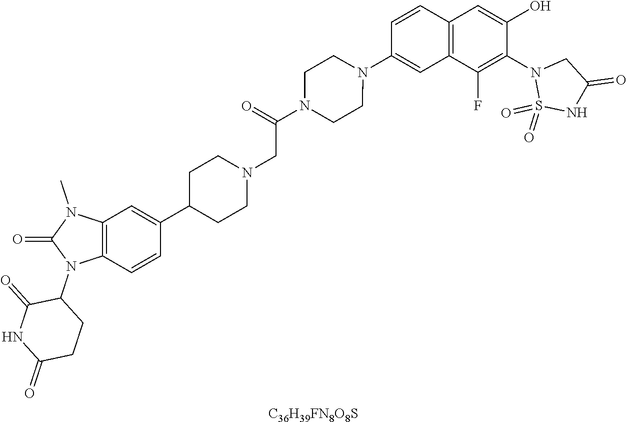 [SMILES: Cn1c(=O)n(C2CCC(=O)NC2=O)c2ccc(C3CCN(CC(=O)N4CCN(c5ccc6cc(O)c(N7CC(=O)NS7(=O)=O)c(F)c6c5)CC4)CC3)cc21 ; "C36H39FN8O8S"]

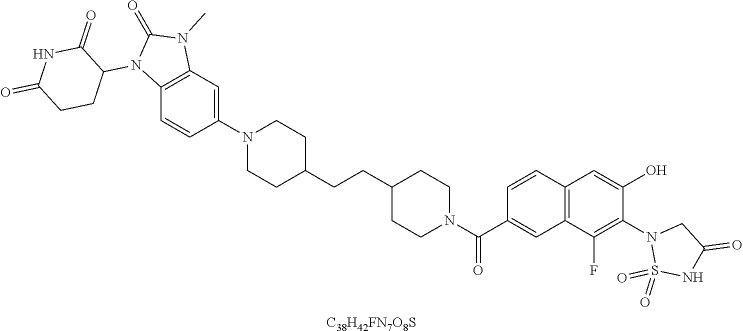 [SMILES: Cn1c(=O)n(C2CCC(=O)NC2=O)c2ccc(N3CCC(CCC4CCN(C(=O)c5ccc6cc(O)c(N7CC(=O)NS7(=O)=O)c(F)c6c5)CC4)CC3)cc21 ; "C38H42FN7O8S"]

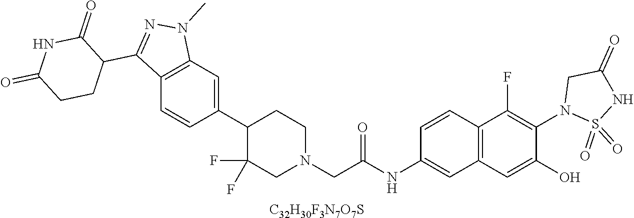

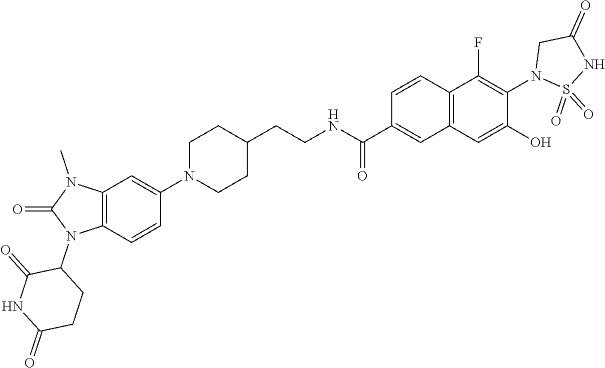 [SMILES: Cn1c(=O)n(C2CCC(=O)NC2=O)c2ccc(N3CCC(CCNC(=O)c4ccc5c(F)c(N6CC(=O)NS6(=O)=O)c(O)cc5c4)CC3)cc21]

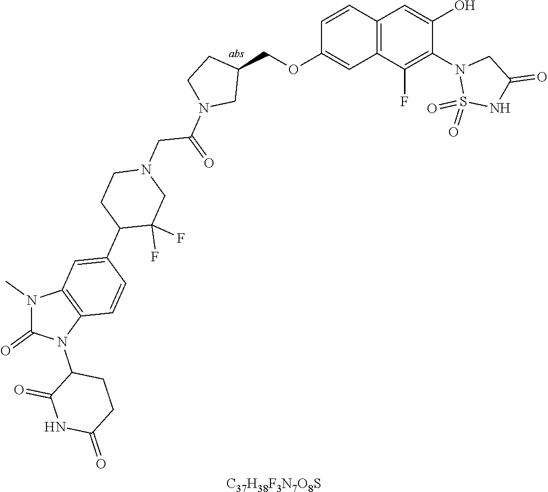 [SMILES: Cn1c(=O)n(C2CCC(=O)NC2=O)c2ccc(C3CCN(CC(=O)N4CC[C@@H](COc5ccc6cc(O)c(N7CC(=O)NS7(=O)=O)c(F)c6c5)C4)CC3(F)F)cc21 ; "C37H38F3N7O8S"]

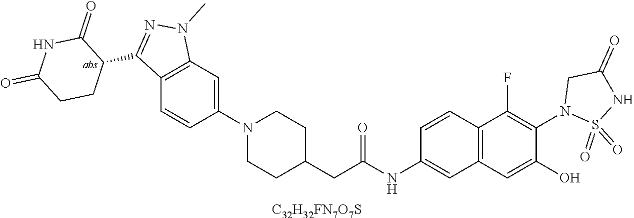

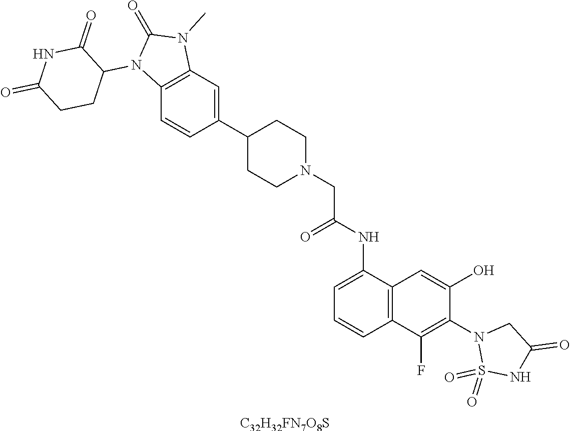 [SMILES: Cn1c(=O)n(C2CCC(=O)NC2=O)c2ccc(C3CCN(CC(=O)Nc4cccc5c(F)c(N6CC(=O)NS6(=O)=O)c(O)cc45)CC3)cc21 ; "C32H32FN7O8S"]

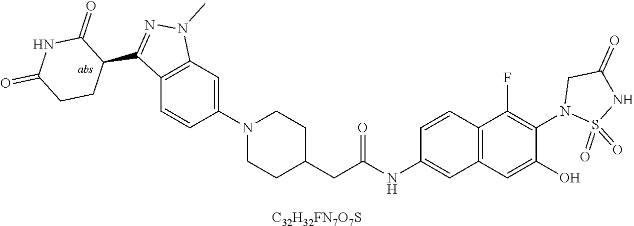

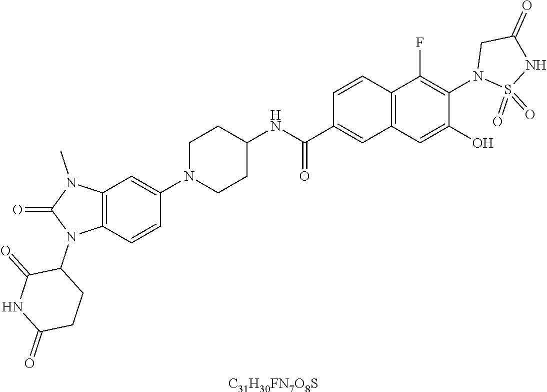 [SMILES: Cn1c(=O)n(C2CCC(=O)NC2=O)c2ccc(N3CCC(NC(=O)c4ccc5c(F)c(N6CC(=O)NS6(=O)=O)c(O)cc5c4)CC3)cc21 ; "C31H30FN7O8S"]

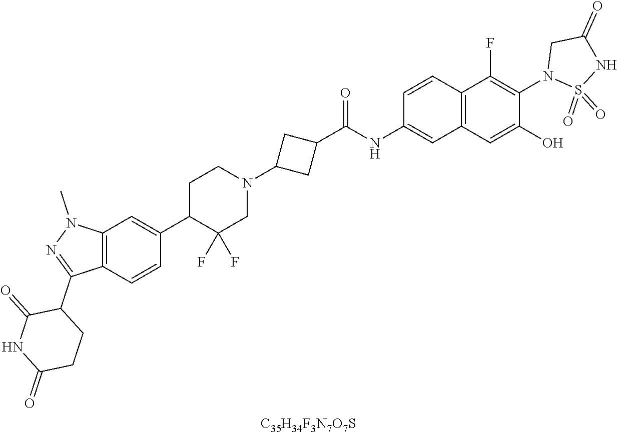 [SMILES: Cn1nc(C2CCC(=O)NC2=O)c2ccc(C3CCN(C4CC(C(=O)Nc5ccc6c(F)c(N7CC(=O)NS7(=O)=O)c(O)cc6c5)C4)CC3(F)F)cc21 ; "C35H34F3N7O7S"]

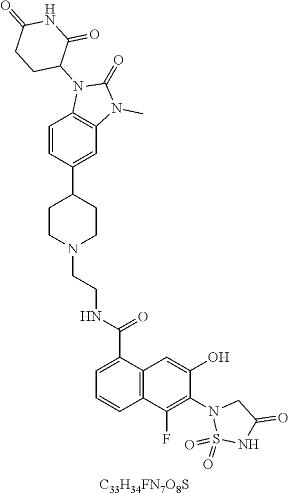 [SMILES: Cn1c(=O)n(C2CCC(=O)NC2=O)c2ccc(C3CCN(CCNC(=O)c4cccc5c(F)c(N6CC(=O)NS6(=O)=O)c(O)cc45)CC3)cc21 ; "C33H34FN7O8S"]

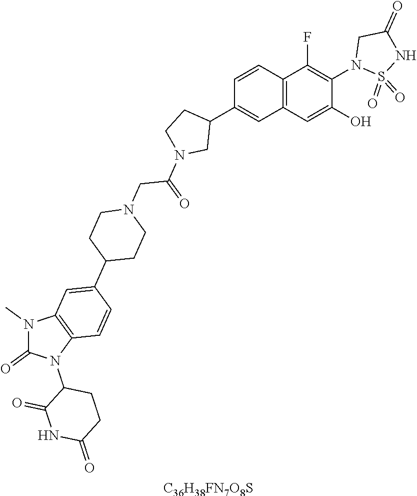 [SMILES: Cn1c(=O)n(C2CCC(=O)NC2=O)c2ccc(C3CCN(CC(=O)N4CCC(c5ccc6c(F)c(N7CC(=O)NS7(=O)=O)c(O)cc6c5)C4)CC3)cc21 ; "C36H38FN7O8S"]

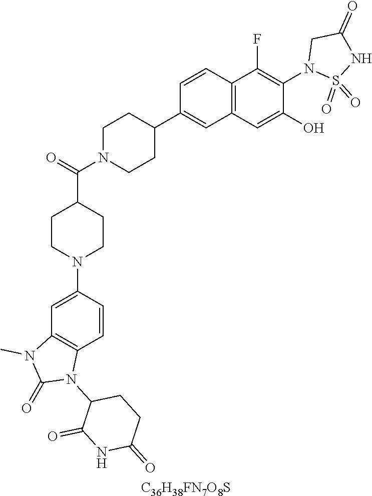

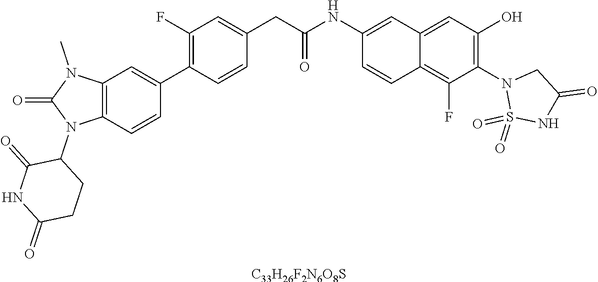 [SMILES: Cn1c(=O)n(C2CCC(=O)NC2=O)c2ccc(-c3ccc(CC(=O)Nc4ccc5c(F)c(N6CC(=O)NS6(=O)=O)c(O)cc5c4)cc3F)cc21 ; "C33H26F2N6O8S"]

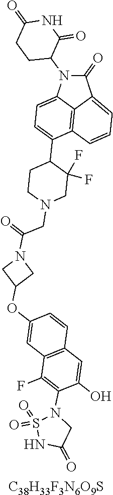 [SMILES: O=C1CCC(N2C(=O)c3cccc4c(C5CCN(CC(=O)N6CC(Oc7ccc8cc(O)c(N9CC(=O)NS9(=O)=O)c(F)c8c7)C6)CC5(F)F)ccc2c34)C(=O)N1 ; "C38H33F3N6O9S"]

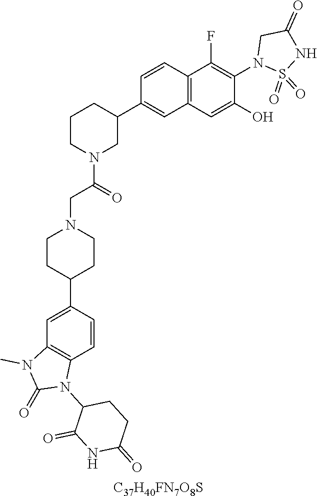

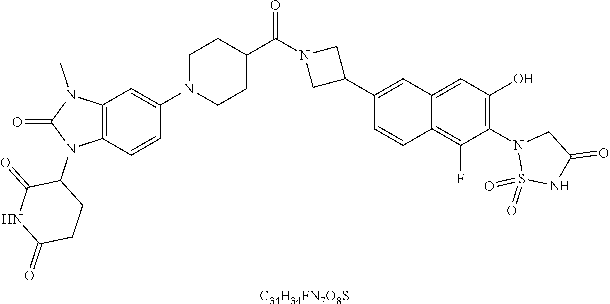 [SMILES: Cn1c(=O)n(C2CCC(=O)NC2=O)c2ccc(N3CCC(C(=O)N4CC(c5ccc6c(F)c(N7CC(=O)NS7(=O)=O)c(O)cc6c5)C4)CC3)cc21 ; "C34H34FN7O8S"]

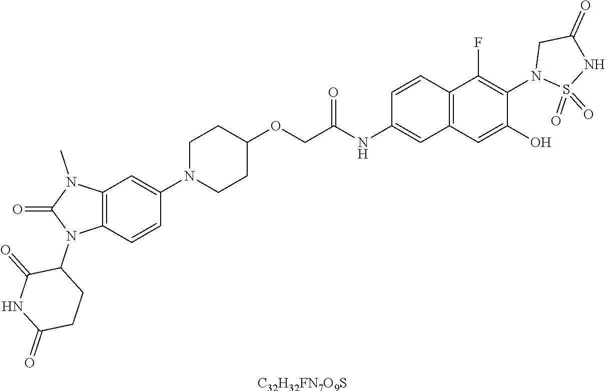 [SMILES: Cn1c(=O)n(C2CCC(=O)NC2=O)c2ccc(N3CCC(OCC(=O)Nc4ccc5c(F)c(N6CC(=O)NS6(=O)=O)c(O)cc5c4)CC3)cc21 ; "C32H32FN7O9S"]

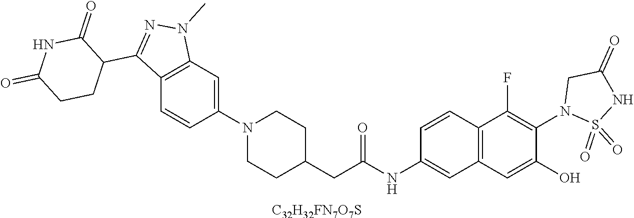

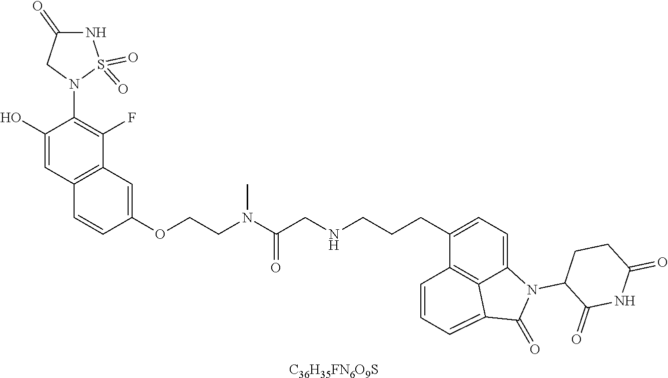 [SMILES: CN(CCOc1ccc2cc(O)c(N3CC(=O)NS3(=O)=O)c(F)c2c1)C(=O)CNCCCc1ccc2c3c(cccc13)C(=O)N2C1CCC(=O)NC1=O ; "C36H35FN6O9S"]

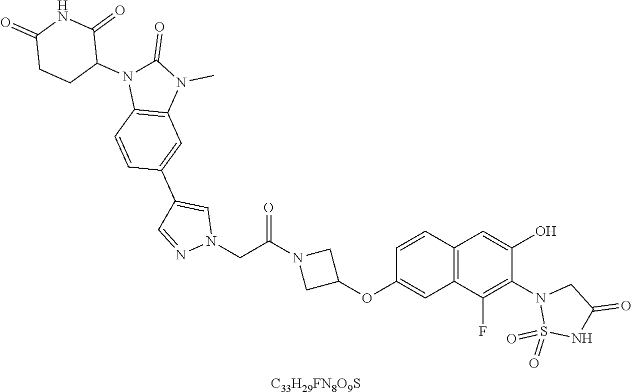 [SMILES: Cn1c(=O)n(C2CCC(=O)NC2=O)c2ccc(-c3cnn(CC(=O)N4CC(Oc5ccc6cc(O)c(N7CC(=O)NS7(=O)=O)c(F)c6c5)C4)c3)cc21 ; "C33H29FN8O9S"]

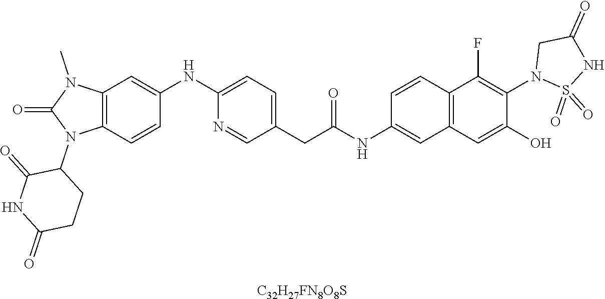 [SMILES: Cn1c(=O)n(C2CCC(=O)NC2=O)c2ccc(Nc3ccc(CC(=O)Nc4ccc5c(F)c(N6CC(=O)NS6(=O)=O)c(O)cc5c4)cn3)cc21 ; "C32H27FN8O8S"]

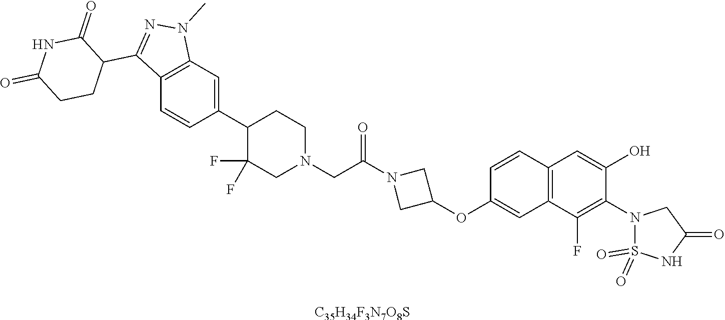 [SMILES: Cn1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21 ; "C35H34F3N7O8S"]

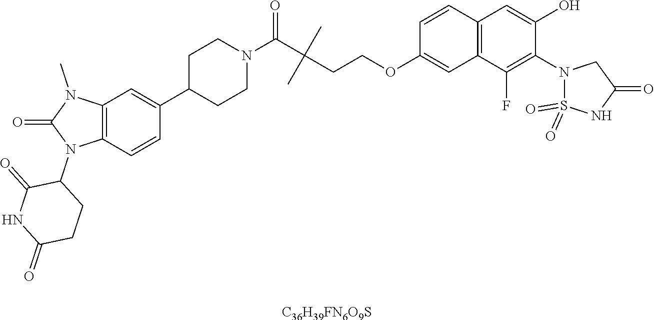 [SMILES: Cn1c(=O)n(C2CCC(=O)NC2=O)c2ccc(C3CCN(C(=O)C(C)(C)CCOc4ccc5cc(O)c(N6CC(=O)NS6(=O)=O)c(F)c5c4)CC3)cc21 ; "C36H39FN6O9S"]

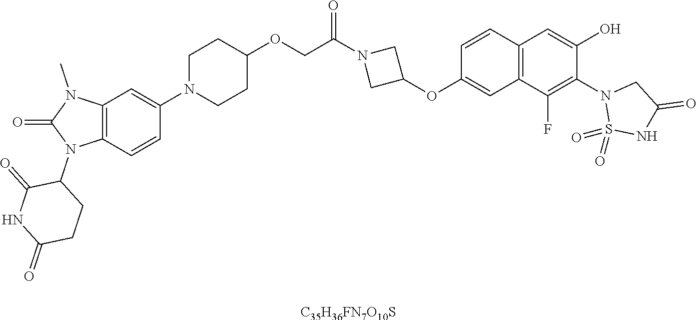 [SMILES: Cn1c(=O)n(C2CCC(=O)NC2=O)c2ccc(N3CCC(OCC(=O)N4CC(Oc5ccc6cc(O)c(N7CC(=O)NS7(=O)=O)c(F)c6c5)C4)CC3)cc21 ; "C35H36FN7O10S"]

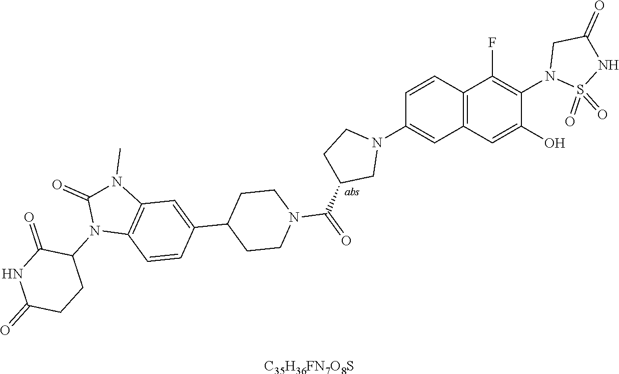 [SMILES: Cn1c(=O)n(C2CCC(=O)NC2=O)c2ccc(C3CCN(C(=O)[C@@H]4CCN(c5ccc6c(F)c(N7CC(=O)NS7(=O)=O)c(O)cc6c5)C4)CC3)cc21 ; "C35H36FN7O8S"]

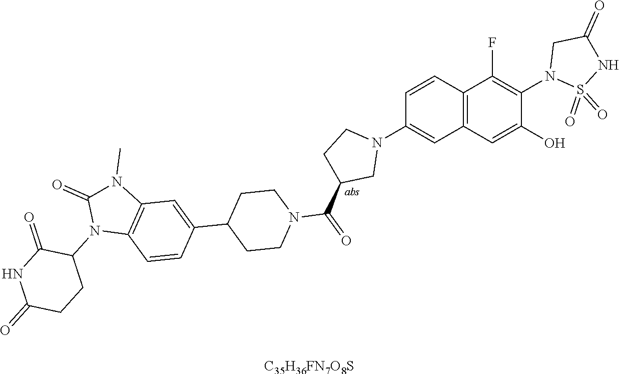 [SMILES: Cn1c(=O)n(C2CCC(=O)NC2=O)c2ccc(C3CCN(C(=O)[C@H]4CCN(c5ccc6c(F)c(N7CC(=O)NS7(=O)=O)c(O)cc6c5)C4)CC3)cc21 ; "C35H36FN7O8S"]

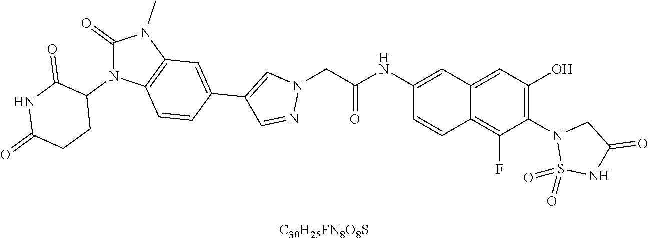 [SMILES: Cn1c(=O)n(C2CCC(=O)NC2=O)c2ccc(-c3cnn(CC(=O)Nc4ccc5c(F)c(N6CC(=O)NS6(=O)=O)c(O)cc5c4)c3)cc21 ; "C30H25FN8O8S"]

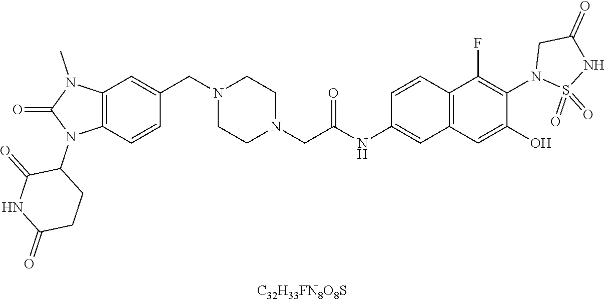 [SMILES: Cn1c(=O)n(C2CCC(=O)NC2=O)c2ccc(CN3CCN(CC(=O)Nc4ccc5c(F)c(N6CC(=O)NS6(=O)=O)c(O)cc5c4)CC3)cc21 ; "C32H33FN8O8S"]

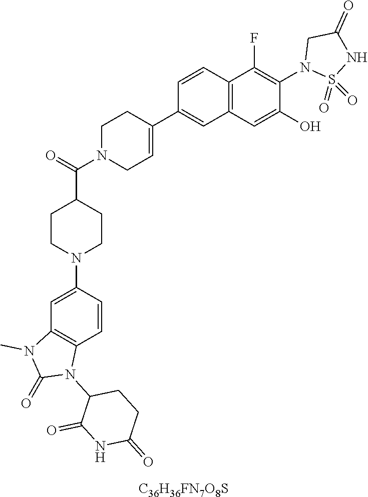

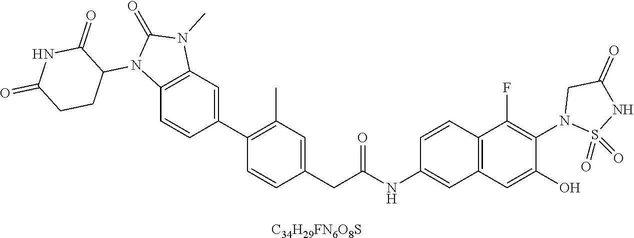

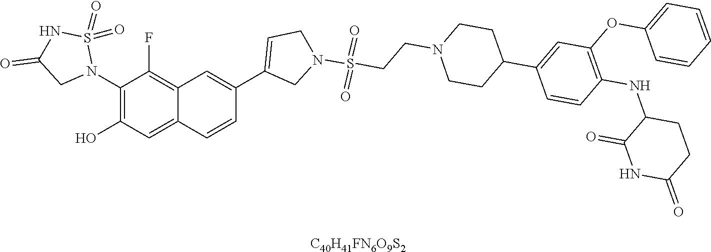 [SMILES: O=C1CCC(Nc2ccc(C3CCN(CCS(=O)(=O)N4CC=C(c5ccc6cc(O)c(N7CC(=O)NS7(=O)=O)c(F)c6c5)C4)CC3)cc2Oc2ccccc2)C(=O)N1 ; "C40H41FN6O9S2"]

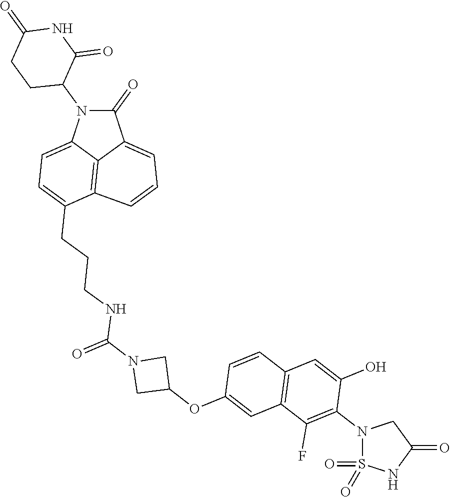 [SMILES: O=C1CCC(N2C(=O)c3cccc4c(CCCNC(=O)N5CC(Oc6ccc7cc(O)c(N8CC(=O)NS8(=O)=O)c(F)c7c6)C5)ccc2c34)C(=O)N1]

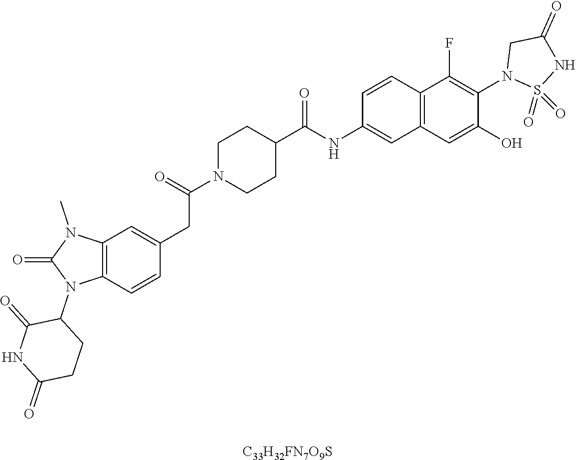 [SMILES: Cn1c(=O)n(C2CCC(=O)NC2=O)c2ccc(CC(=O)N3CCC(C(=O)Nc4ccc5c(F)c(N6CC(=O)NS6(=O)=O)c(O)cc5c4)CC3)cc21 ; "C33H32FN7O9S"]

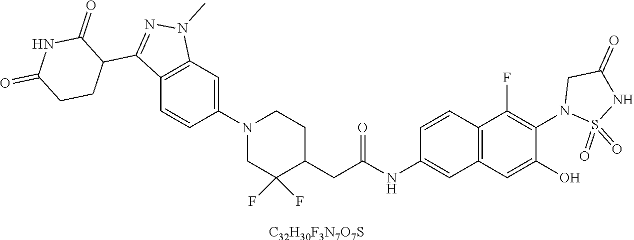

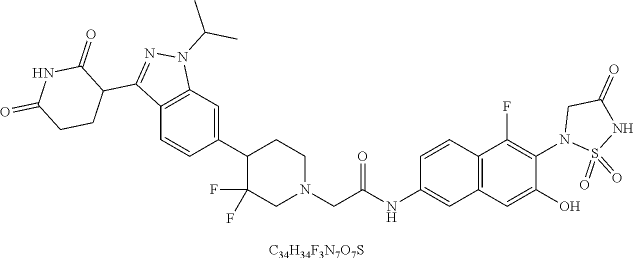

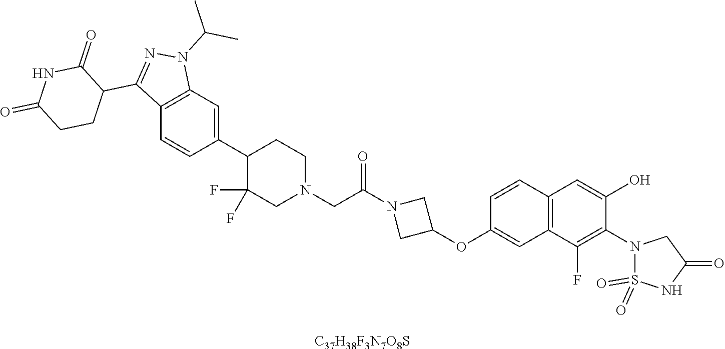 [SMILES: CC(C)n1nc(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21 ; "C37H38F3N7O8S"]

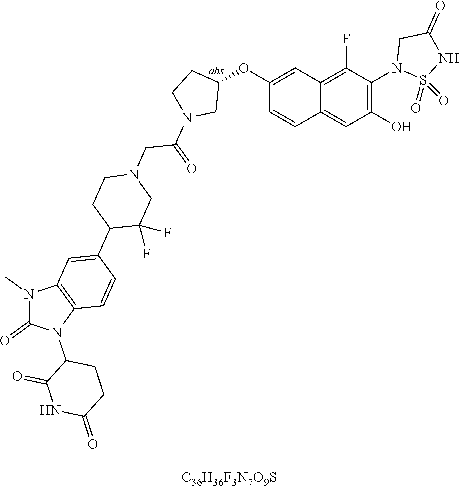 [SMILES: Cn1c(=O)n(C2CCC(=O)NC2=O)c2ccc(C3CCN(CC(=O)N4CC[C@H](Oc5ccc6cc(O)c(N7CC(=O)NS7(=O)=O)c(F)c6c5)C4)CC3(F)F)cc21 ; "C36H36F3N7O9S"]

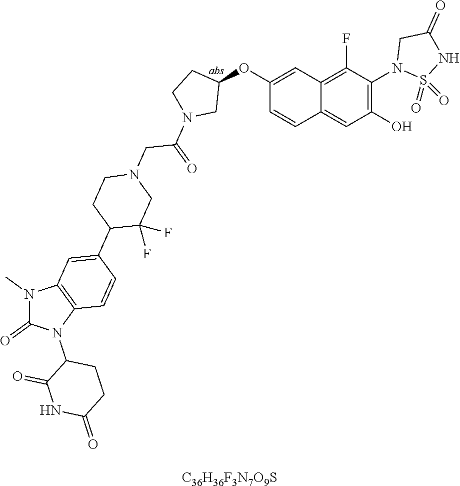 [SMILES: Cn1c(=O)n(C2CCC(=O)NC2=O)c2ccc(C3CCN(CC(=O)N4CC[C@@H](Oc5ccc6cc(O)c(N7CC(=O)NS7(=O)=O)c(F)c6c5)C4)CC3(F)F)cc21 ; "C36H36F3N7O9S"]

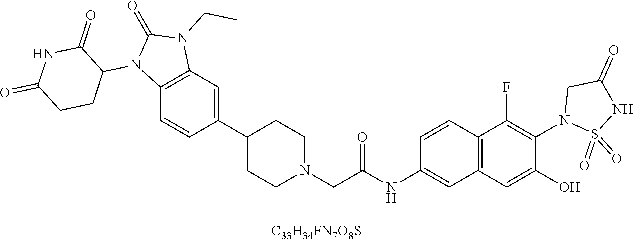

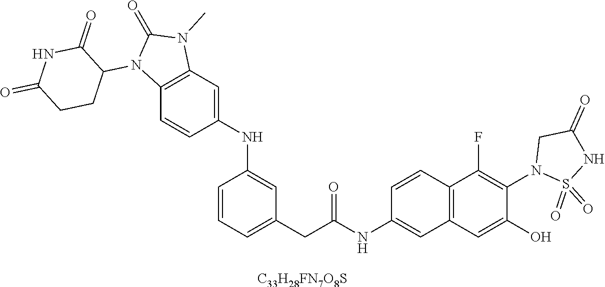

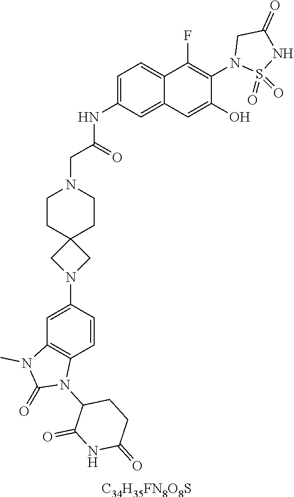 [SMILES: Cn1c(=O)n(C2CCC(=O)NC2=O)c2ccc(N3CC4(CCN(CC(=O)Nc5ccc6c(F)c(N7CC(=O)NS7(=O)=O)c(O)cc6c5)CC4)C3)cc21 ; "C34H35FN8O8S"]

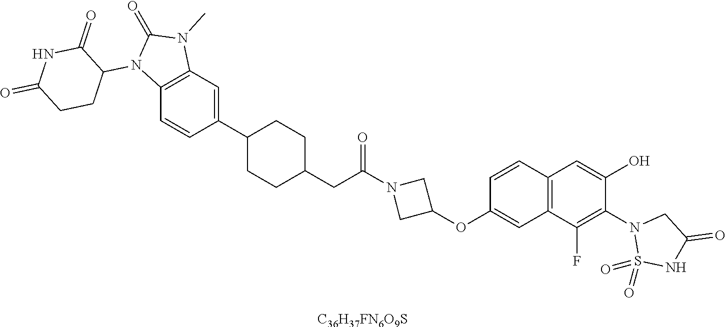 [SMILES: Cn1c(=O)n(C2CCC(=O)NC2=O)c2ccc(C3CCC(CC(=O)N4CC(Oc5ccc6cc(O)c(N7CC(=O)NS7(=O)=O)c(F)c6c5)C4)CC3)cc21 ; "C36H37FN6O9S"]

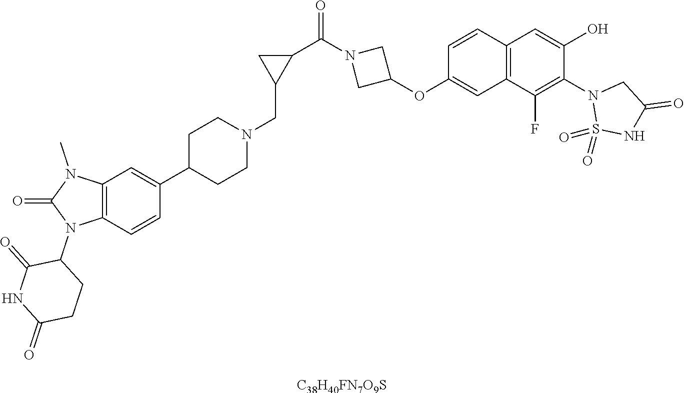 [SMILES: Cn1c(=O)n(C2CCC(=O)NC2=O)c2ccc(C3CCN(CC4CC4C(=O)N4CC(Oc5ccc6cc(O)c(N7CC(=O)NS7(=O)=O)c(F)c6c5)C4)CC3)cc21 ; "C38H40FN7O9S"]

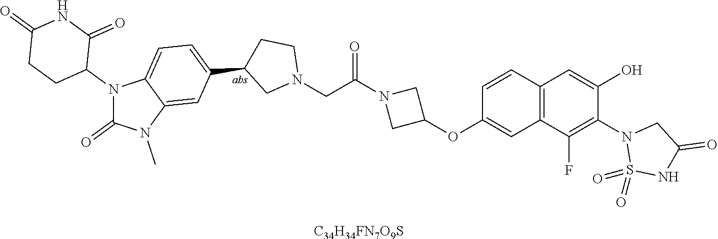 [SMILES: Cn1c(=O)n(C2CCC(=O)NC2=O)c2ccc([C@H]3CCN(CC(=O)N4CC(Oc5ccc6cc(O)c(N7CC(=O)NS7(=O)=O)c(F)c6c5)C4)C3)cc21 ; "C34H34FN7O9S"]

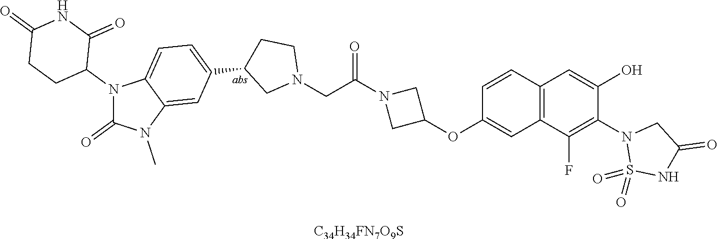 [SMILES: Cn1c(=O)n(C2CCC(=O)NC2=O)c2ccc([C@@H]3CCN(CC(=O)N4CC(Oc5ccc6cc(O)c(N7CC(=O)NS7(=O)=O)c(F)c6c5)C4)C3)cc21 ; "C34H34FN7O9S"]

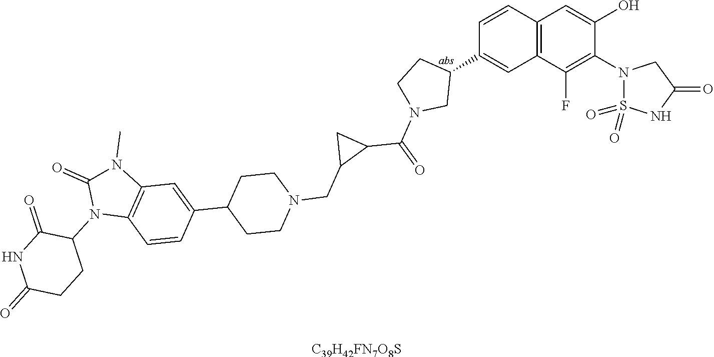 [SMILES: Cn1c(=O)n(C2CCC(=O)NC2=O)c2ccc(C3CCN(CC4CC4C(=O)N4CC[C@H](c5ccc6cc(O)c(N7CC(=O)NS7(=O)=O)c(F)c6c5)C4)CC3)cc21 ; "C39H42FN7O8S"]

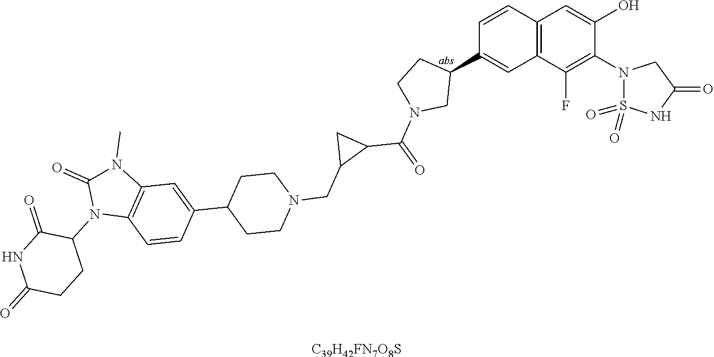 [SMILES: Cn1c(=O)n(C2CCC(=O)NC2=O)c2ccc(C3CCN(CC4CC4C(=O)N4CC[C@@H](c5ccc6cc(O)c(N7CC(=O)NS7(=O)=O)c(F)c6c5)C4)CC3)cc21 ; "C39H42FN7O8S"]

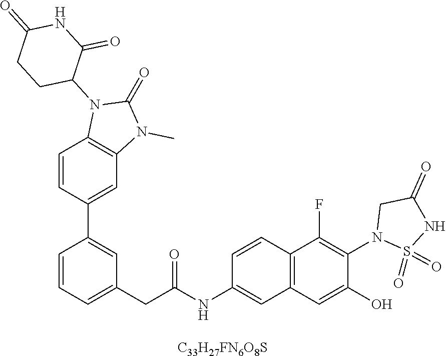 [SMILES: Cn1c(=O)n(C2CCC(=O)NC2=O)c2ccc(-c3cccc(CC(=O)Nc4ccc5c(F)c(N6CC(=O)NS6(=O)=O)c(O)cc5c4)c3)cc21 ; "C33H27FN6O8S"]

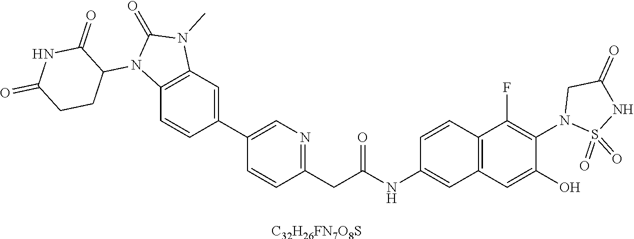

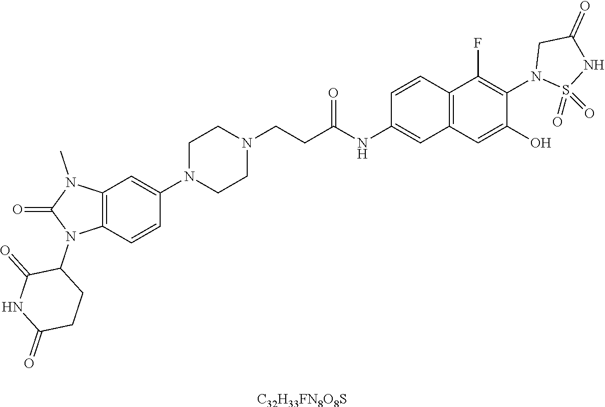 [SMILES: Cn1c(=O)n(C2CCC(=O)NC2=O)c2ccc(N3CCN(CCC(=O)Nc4ccc5c(F)c(N6CC(=O)NS6(=O)=O)c(O)cc5c4)CC3)cc21 ; "C32H33FN8O8S"]

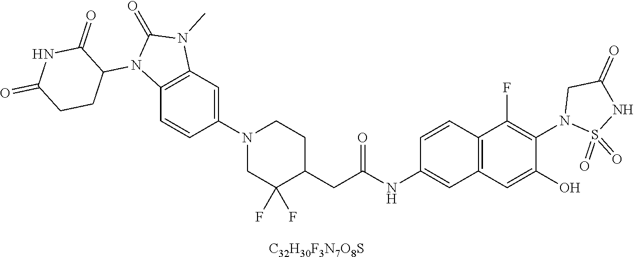

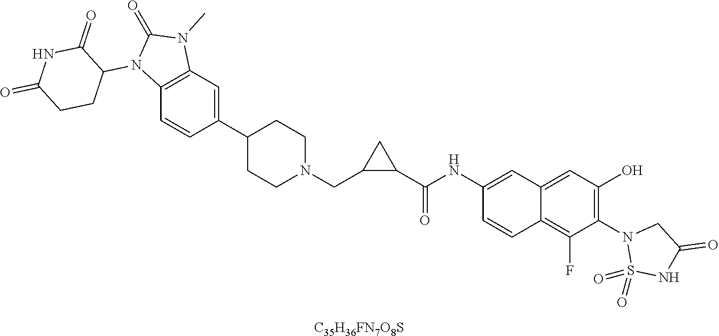 [SMILES: Cn1c(=O)n(C2CCC(=O)NC2=O)c2ccc(C3CCN(CC4CC4C(=O)Nc4ccc5c(F)c(N6CC(=O)NS6(=O)=O)c(O)cc5c4)CC3)cc21 ; "C35H36FN7O8S"]

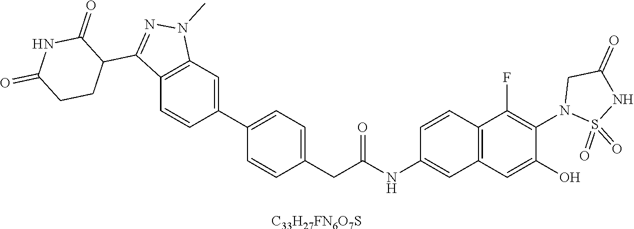

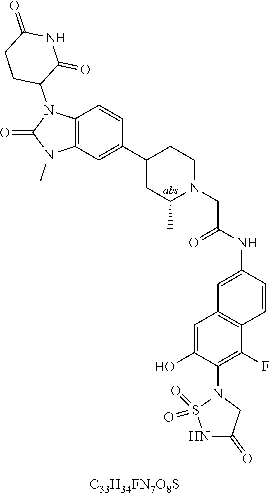 [SMILES: C[C@@H]1CC(c2ccc3c(c2)n(C)c(=O)n3C2CCC(=O)NC2=O)CCN1CC(=O)Nc1ccc2c(F)c(N3CC(=O)NS3(=O)=O)c(O)cc2c1 ; "C33H34FN7O8S"]

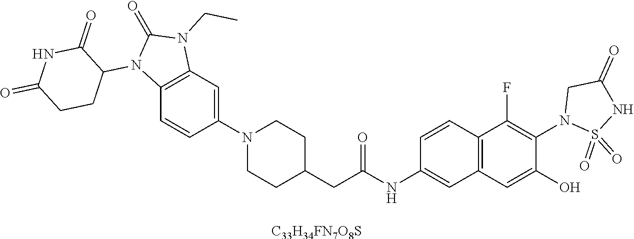

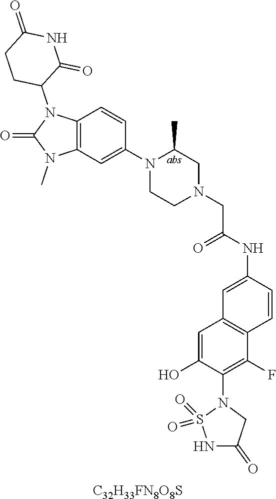 [SMILES: C[C@H]1CN(CC(=O)Nc2ccc3c(F)c(N4CC(=O)NS4(=O)=O)c(O)cc3c2)CCN1c1ccc2c(c1)n(C)c(=O)n2C1CCC(=O)NC1=O ; "C32H33FN8O8S"]

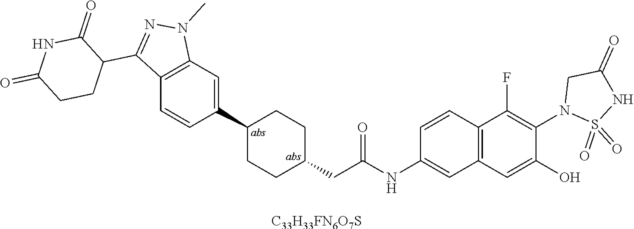

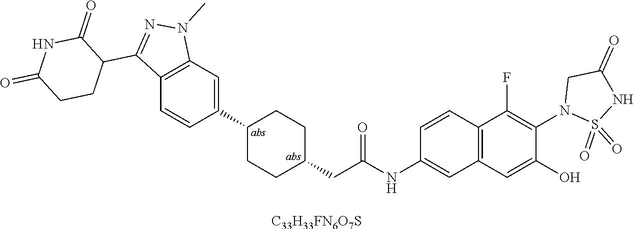

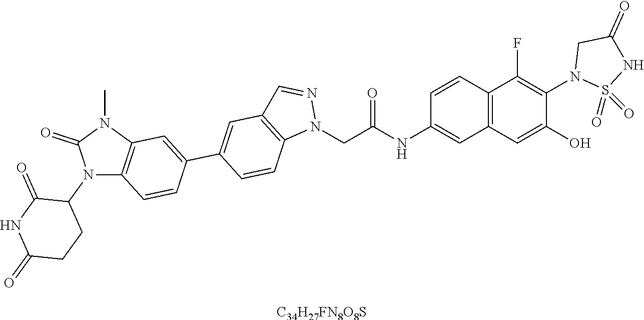 [SMILES: Cn1c(=O)n(C2CCC(=O)NC2=O)c2ccc(-c3ccc4c(cnn4CC(=O)Nc4ccc5c(F)c(N6CC(=O)NS6(=O)=O)c(O)cc5c4)c3)cc21 ; "C34H27FN8O8S"]

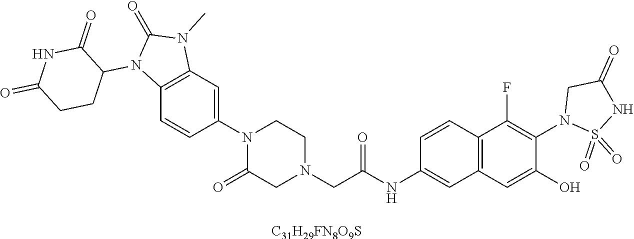

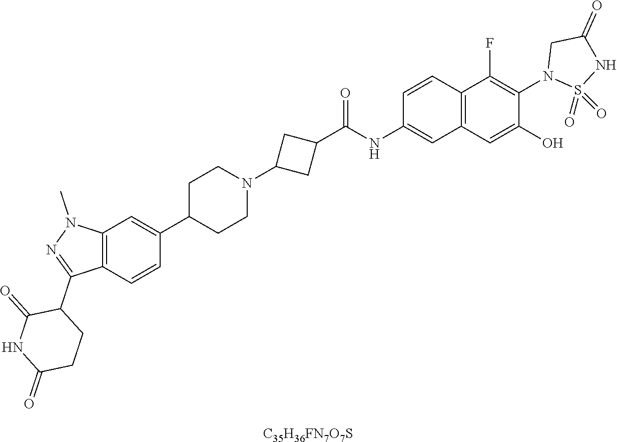 [SMILES: Cn1nc(C2CCC(=O)NC2=O)c2ccc(C3CCN(C4CC(C(=O)Nc5ccc6c(F)c(N7CC(=O)NS7(=O)=O)c(O)cc6c5)C4)CC3)cc21 ; "C35H36FN7O7S"]

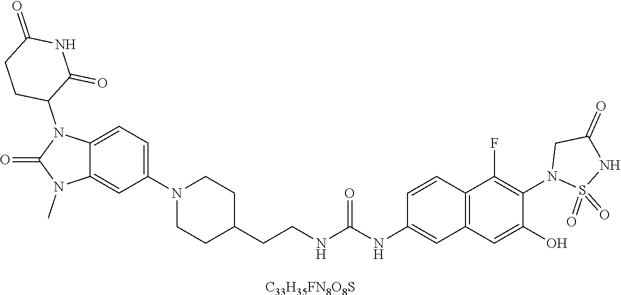

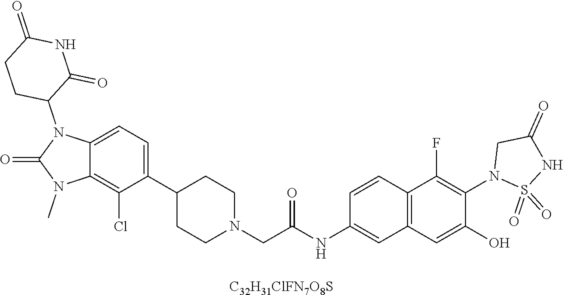 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3)c(Cl)c21 ; "C32H31ClFN7O8S"]

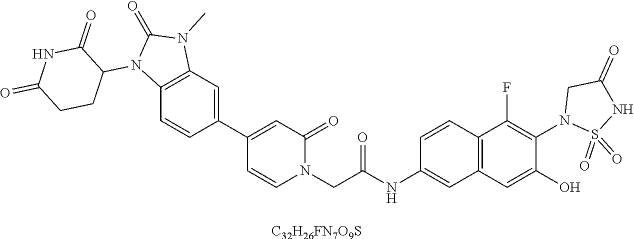

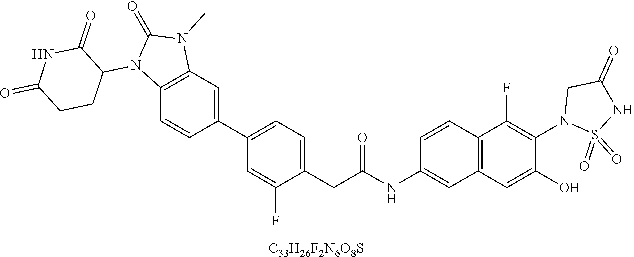

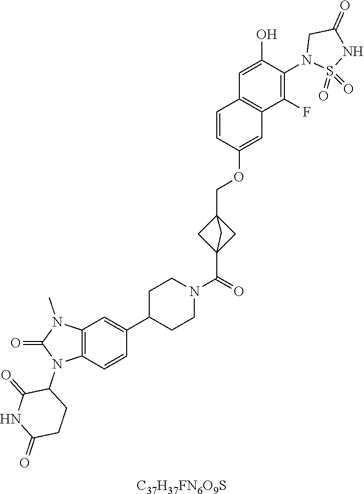 [SMILES: Cn1c(=O)n(C2CCC(=O)NC2=O)c2ccc(C3CCN(C(=O)C45CC(COc6ccc7cc(O)c(N8CC(=O)NS8(=O)=O)c(F)c7c6)(C4)C5)CC3)cc21 ; "C37H37FN6O9S"]

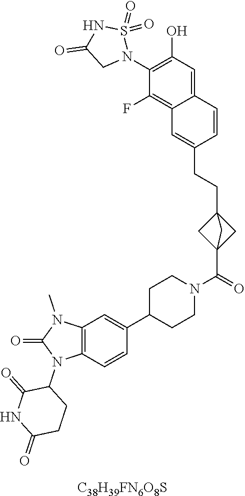 [SMILES: Cn1c(=O)n(C2CCC(=O)NC2=O)c2ccc(C3CCN(C(=O)C45CC(CCc6ccc7cc(O)c(N8CC(=O)NS8(=O)=O)c(F)c7c6)(C4)C5)CC3)cc21 ; "C38H39FN6O8S"]

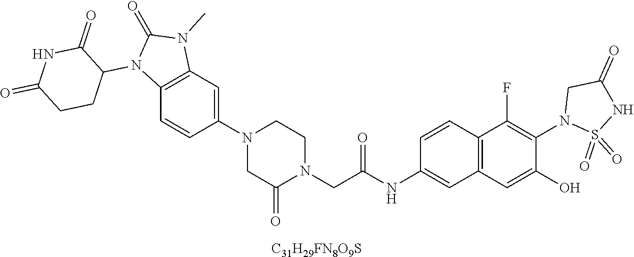

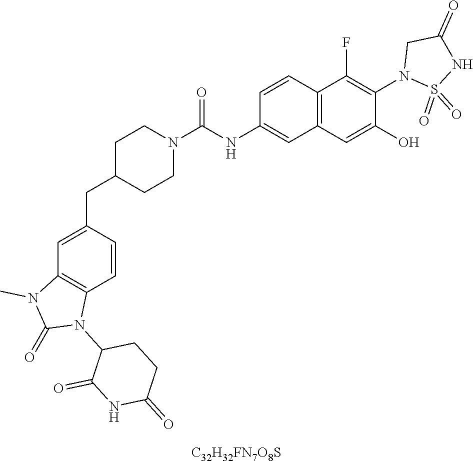

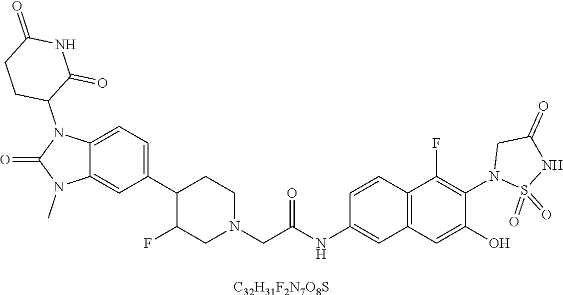 [SMILES: Cn1c(=O)n(C2CCC(=O)NC2=O)c2ccc(C3CCN(CC(=O)Nc4ccc5c(F)c(N6CC(=O)NS6(=O)=O)c(O)cc5c4)CC3F)cc21 ; "C32H31F2N7O8S"]

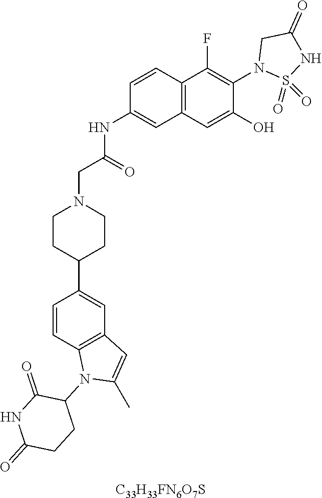 [SMILES: Cc1cc2cc(C3CCN(CC(=O)Nc4ccc5c(F)c(N6CC(=O)NS6(=O)=O)c(O)cc5c4)CC3)ccc2n1C1CCC(=O)NC1=O ; "C33H33FN6O7S"]

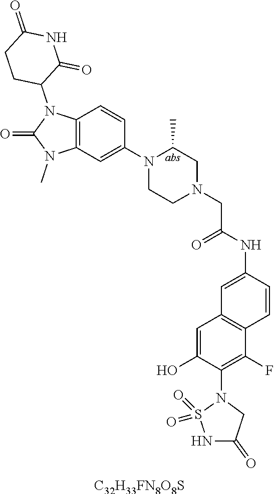 [SMILES: C[C@@H]1CN(CC(=O)Nc2ccc3c(F)c(N4CC(=O)NS4(=O)=O)c(O)cc3c2)CCN1c1ccc2c(c1)n(C)c(=O)n2C1CCC(=O)NC1=O ; "C32H33FN8O8S"]

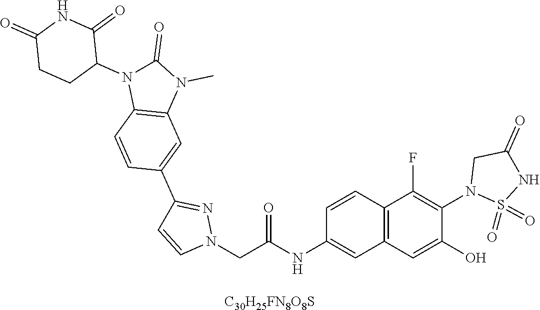

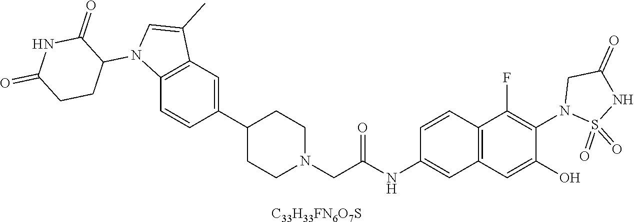

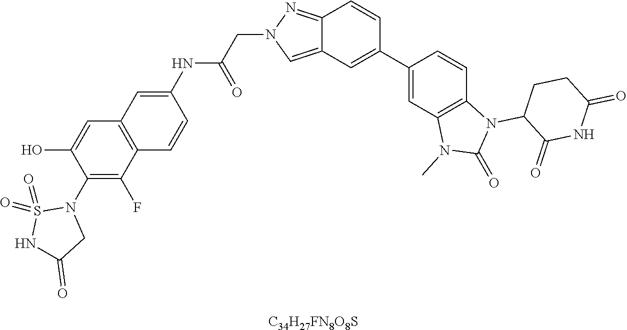 [SMILES: Cn1c(=O)n(C2CCC(=O)NC2=O)c2ccc(-c3ccc4nn(CC(=O)Nc5ccc6c(F)c(N7CC(=O)NS7(=O)=O)c(O)cc6c5)cc4c3)cc21 ; "C34H27FN8O8S"]

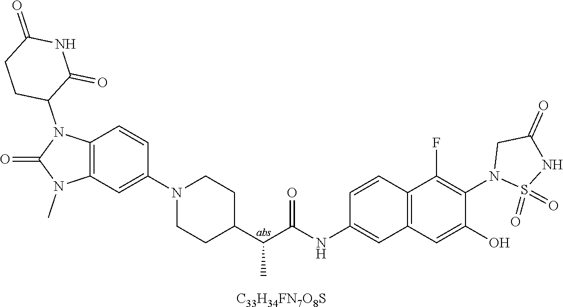

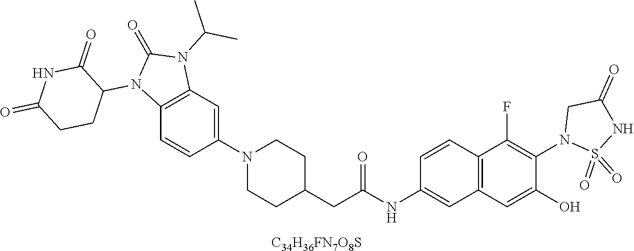

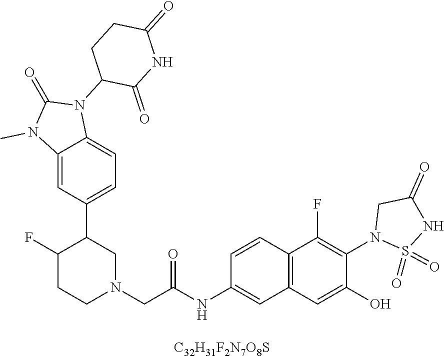 [SMILES: Cn1c(=O)n(C2CCC(=O)NC2=O)c2ccc(C3CN(CC(=O)Nc4ccc5c(F)c(N6CC(=O)NS6(=O)=O)c(O)cc5c4)CCC3F)cc21 ; "C32H31F2N7O8S"]

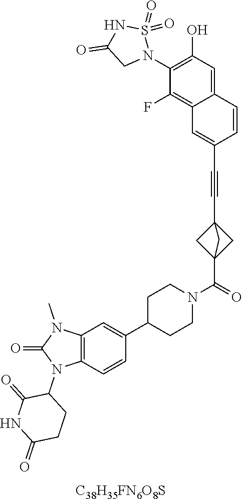 [SMILES: Cn1c(=O)n(C2CCC(=O)NC2=O)c2ccc(C3CCN(C(=O)C45CC(C#Cc6ccc7cc(O)c(N8CC(=O)NS8(=O)=O)c(F)c7c6)(C4)C5)CC3)cc21 ; "C38H35FN6O8S"]

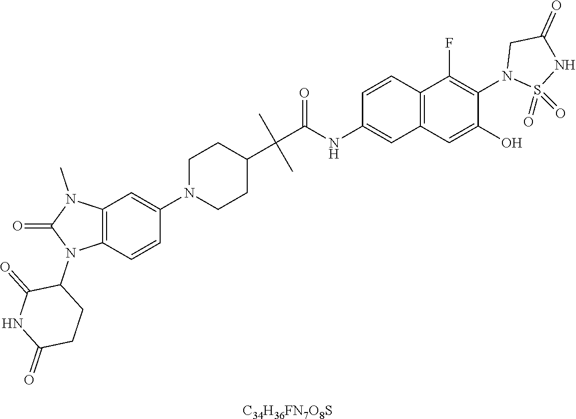 [SMILES: Cn1c(=O)n(C2CCC(=O)NC2=O)c2ccc(N3CCC(C(C)(C)C(=O)Nc4ccc5c(F)c(N6CC(=O)NS6(=O)=O)c(O)cc5c4)CC3)cc21 ; "C34H36FN7O8S"]

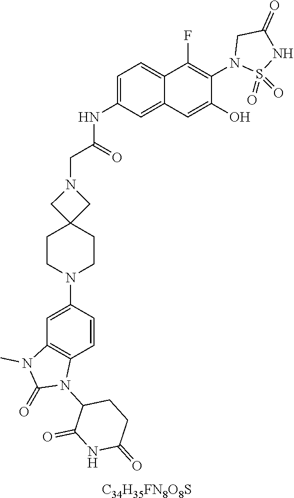 [SMILES: Cn1c(=O)n(C2CCC(=O)NC2=O)c2ccc(N3CCC4(CC3)CN(CC(=O)Nc3ccc5c(F)c(N6CC(=O)NS6(=O)=O)c(O)cc5c3)C4)cc21 ; "C34H35FN8O8S"]

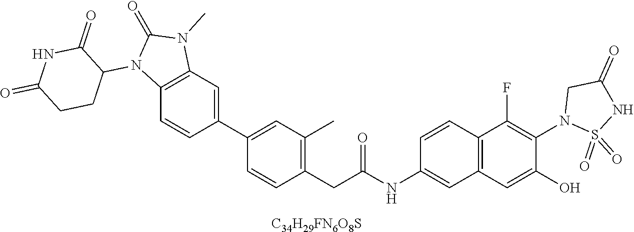

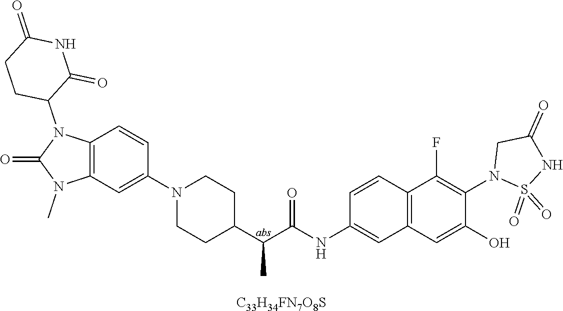

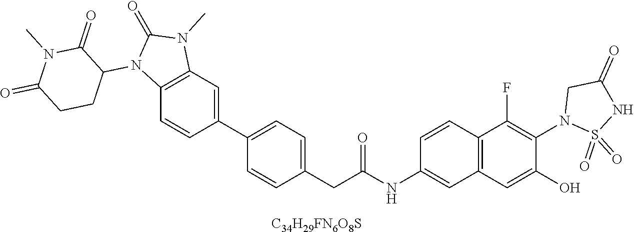

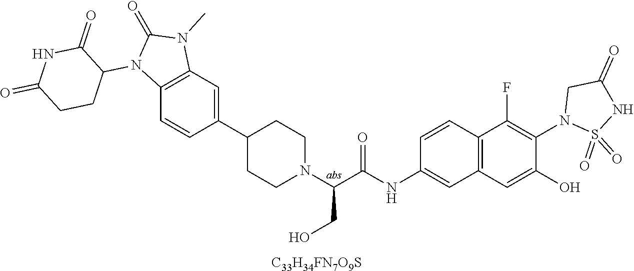

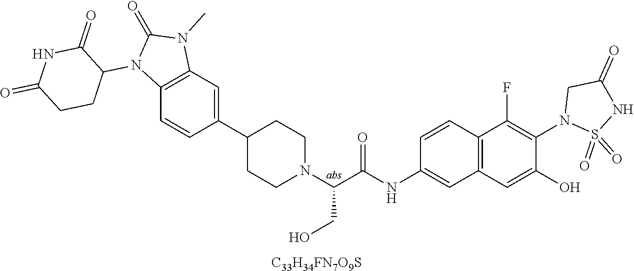

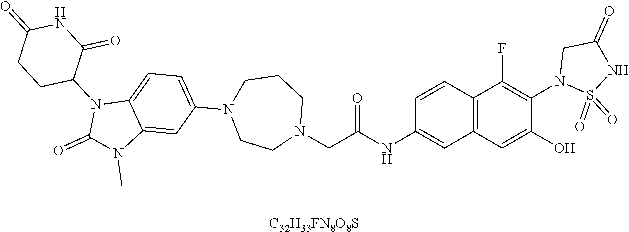 [SMILES: Cn1c(=O)n(C2CCC(=O)NC2=O)c2ccc(N3CCCN(CC(=O)Nc4ccc5c(F)c(N6CC(=O)NS6(=O)=O)c(O)cc5c4)CC3)cc21 ; "C32H33FN8O8S"]

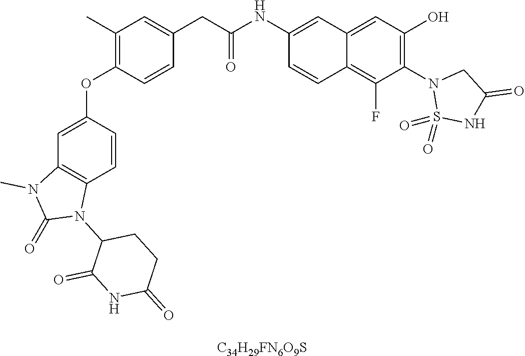 [SMILES: Cc1cc(CC(=O)Nc2ccc3c(F)c(N4CC(=O)NS4(=O)=O)c(O)cc3c2)ccc1Oc1ccc2c(c1)n(C)c(=O)n2C1CCC(=O)NC1=O ; "C34H29FN6O9S"]

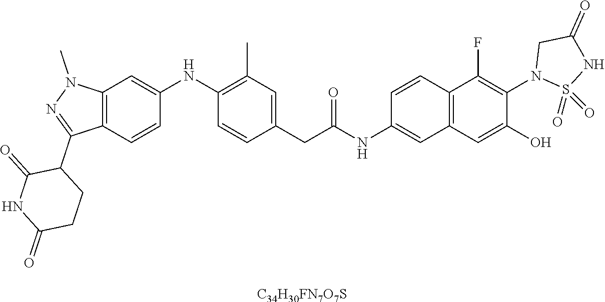 [SMILES: Cc1cc(CC(=O)Nc2ccc3c(F)c(N4CC(=O)NS4(=O)=O)c(O)cc3c2)ccc1Nc1ccc2c(C3CCC(=O)NC3=O)nn(C)c2c1 ; "C34H30FN7O7S"]

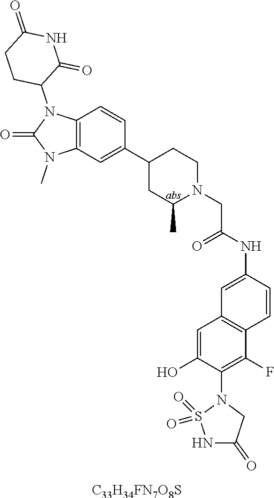 [SMILES: C[C@H]1CC(c2ccc3c(c2)n(C)c(=O)n3C2CCC(=O)NC2=O)CCN1CC(=O)Nc1ccc2c(F)c(N3CC(=O)NS3(=O)=O)c(O)cc2c1 ; "C33H34FN7O8S"]

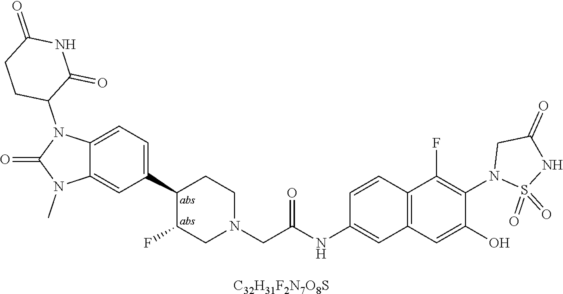 [SMILES: Cn1c(=O)n(C2CCC(=O)NC2=O)c2ccc([C@H]3CCN(CC(=O)Nc4ccc5c(F)c(N6CC(=O)NS6(=O)=O)c(O)cc5c4)C[C@@H]3F)cc21 ; "C32H31F2N7O8S"]

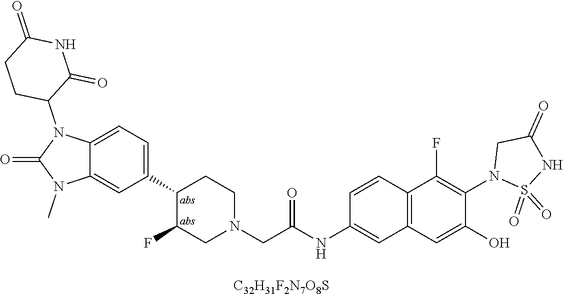 [SMILES: Cn1c(=O)n(C2CCC(=O)NC2=O)c2ccc([C@@H]3CCN(CC(=O)Nc4ccc5c(F)c(N6CC(=O)NS6(=O)=O)c(O)cc5c4)C[C@H]3F)cc21 ; "C32H31F2N7O8S"]

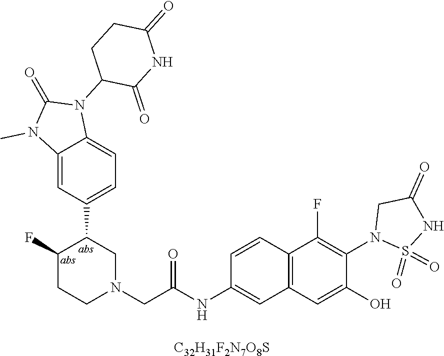 [SMILES: Cn1c(=O)n(C2CCC(=O)NC2=O)c2ccc([C@@H]3CN(CC(=O)Nc4ccc5c(F)c(N6CC(=O)NS6(=O)=O)c(O)cc5c4)CC[C@H]3F)cc21 ; "C32H31F2N7O8S"]

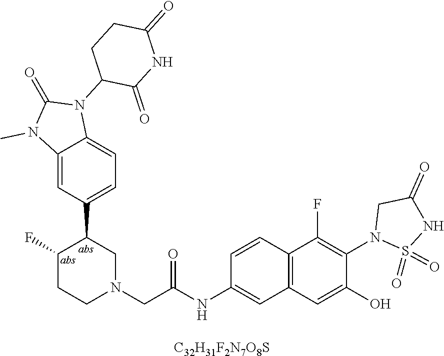 [SMILES: Cn1c(=O)n(C2CCC(=O)NC2=O)c2ccc([C@H]3CN(CC(=O)Nc4ccc5c(F)c(N6CC(=O)NS6(=O)=O)c(O)cc5c4)CC[C@@H]3F)cc21 ; "C32H31F2N7O8S"]

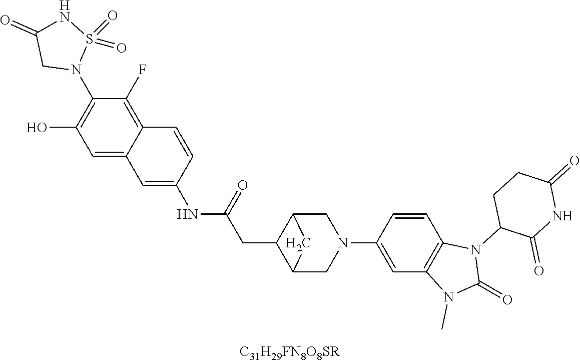 [SMILES: Cn1c(=O)n(C2CCC(=O)NC2=O)c2ccc(N3CC4CC(C3)C4CC(=O)Nc3ccc4c(F)c(N5CC(=O)NS5(=O)=O)c(O)cc4c3)cc21 ; "C31H29FN8O8SR"]

Some embodiments provide a compound of Formula (III):

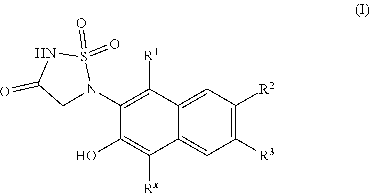

or a pharmaceutically acceptable salt thereof, wherein:
R¹ is hydrogen or halogen;
R² is hydrogen, halogen, C1-C3 alkoxy, C3-C6 cycloalkoxy, C1-C3 haloalkoxy, C3-C5 halocycloalkoxy, C1-C3 alkyl, C1-C3 haloalkyl, C3-C6 cycloalkyl, or -L-Q¹;
R³ is hydrogen, halogen, C1-C3 alkoxy, C3-C5 cycloalkoxy, C1-C3 haloalkoxy, C3-C5 halocycloalkoxy, C1-C3 alkyl, C1-C3 haloalkyl, C3-C5 cycloalkyl, or -L-Q¹;
wherein one of R² and R³ is -L-Q¹ and the other of R² and R³ is not -L-Q¹;
R^{x} is hydrogen or halogen;
L is —U—V—W—X—Y—;
U is a bond, —(NR⁴), —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—, —OR⁵—, —R⁵O—, —NR⁴R⁵—, —R⁵NR⁴—, or —(NR⁴)(C═O)(NR⁴)—;
each R⁴ is independently a hydrogen, C1-C6 alkyl, or C3-C5 cycloalkyl;
R⁵ is C1-C3 alkylene, C3-C7 cycloalkylene, or 4-12 membered heterocyclylene;
V is a bond, —(NR⁴)—, —O—, C1-C6 alkylene, C2-C6 alkenylene, —(C═O)NR⁴—, —(NR⁴)R⁵—, —(NR⁴)(C═O)—, —NH(C═O)NH—, —OR⁵—, —R⁵O—, 4-10-membered heterocyclylene, 5-10 membered heteroarylene, C6-C10 arylene, or C3-C6 cycloalkylene;
W is a bond, C1-C3 alkylene optionally substituted with hydroxyl, C3-C6 cycloalkylene, 4-12 membered heterocyclylene, —O—, —(NR⁴)—, —R⁵(NR⁴)—, —(NR⁴)R⁵—, —(NR⁴)(C═O)—, —R⁵(NR⁴)(C═O)—, —(C═O)(NR⁴)R⁵—, —R⁵(C═O)(NR⁴)—, —(C═O)(NR⁴)—, —R⁵(C═O)—, —(C═O)R⁵—, —(C═O)—, —(S═O)—, or —S(O₂)—;
X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R⁵(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—;
Y is R⁶, R⁶(CR^{A}R^{B})ₚ-Q-, or -Q-(CR^{A}R^{B})ₚR⁶—;
Q is selected from the group consisting of —(NR⁴)—, —O—, and —(CR^{A}R^{B})ₚ—;
p is 0, 1, 2, or 3;
R⁶ is C1-C3 alkylene, C3-C7 cycloalkylene, 4-12 membered heterocyclylene, C6-C10 arylene, or 5-10 membered heteroarylene;
wherein the heterocyclylene, heteroarylene, arylene, and cycloalkylene groups of U, V, W, X, and R⁶ are each optionally substituted with 1-3 substituents independently selected from fluoro, hydroxyl, C1-C6 alkoxy, and C1-C6 alkyl;
each R^{A} and R^{B} is independently hydrogen, fluoro, or C1-C6 alkyl; or
R^{A} and R^{B}, together with the carbon atom to which they are attached, come together to form a C3-C4 cycloalkyl; or
R^{A} and R^{B} combine to form oxo; and
Q¹ is —NH₂, —OH, —CO₂H, —(C═O)Cl, —N₃, or C2-C6 alkyne.

### Pharmaceutical Compositions

Some embodiments provide a pharmaceutical composition comprising a compound of Formula (I), or a pharmaceutically acceptable salt thereof.

### Methods of Treatment

The present disclosure features compounds, compositions, and methods comprising a compound of Formula (I). In some embodiments, the compounds, compositions, and methods described herein are used in the prevention or treatment of a disease. Exemplary diseases include, but are not limited to cancer, type-2 diabetes, metabolic syndrome, obesity, NAFLD, NASH, or another metabolic disease.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General scheme relating to methods of making exemplary compounds of the disclosure are additionally described herein.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

APCI for atmospheric pressure chemical ionization: DCI for desorption chemical ionization: DMSO for dimethyl sulfoxide: ESI for electrospray ionization: HPLC for high performance liquid chromatography: LC/MS for liquid chromatography/mass spectrometry: LED for light-emitting diode; MS for mass spectrum; NMR for nuclear magnetic resonance; psi for pounds per square inch; and TLC for thin-layer chromatography.

### Preparation of Exemplary Intermediates

### 5-(3-(Benzyloxy)-6-bromo-1-fluoronaphthalen-2-yl)-1,2,5-thiadiazolidin-3-one 1,1-dioxide (8)

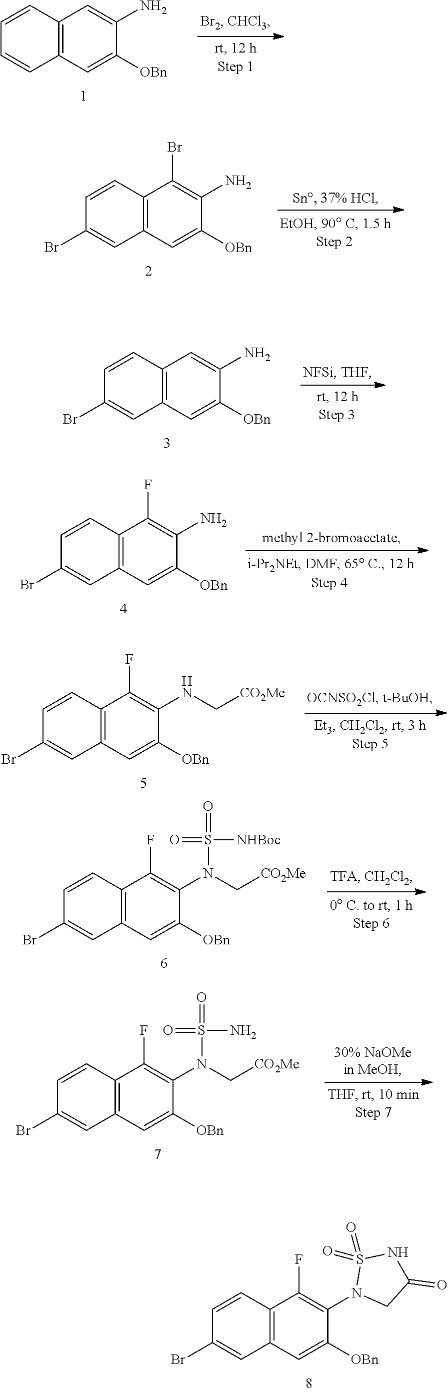

### Step 1: 3-(Benzyloxy)-1,6-dibromonaphthalen-2-amine (2)

3-(Benzyloxy)naphthalen-2-amine ([1092455-29-2]; 20.0 g, 60.2 mmol) in CHCl₃ (300 mL) was treated, dropwise, with Br₂ (6.82 mL, 132 mmol) at RT. After 12 h, the mixture was poured into water and made basic by addition of solid Na₂CO₃ (pH=8). The aqueous mixture was extracted with ethyl acetate (3×200 mL); the organic layers combined, washed with brine (200 mL); then dried with Na₂SO₄, filtered and concentrated to give 3-(benzyloxy)-1,6-dibromonaphthalen-2-amine (23 g, 51 mmol, 85% yield: 90% purity) as a dark solid.

LCMS (TFA: ESI+): m/z 407.9 [M+H]⁺

¹H NMR (400 MHZ, CDCl₃) δ 5.06-5.30 (m, 3H), 7.01 (s, 1H), 7.34-7.52 (m, 7H), 7.76 (d, J=2.0 Hz, 1H), 7.83 (d, J=9.0 Hz, 1H).

### Step 2: 3-(Benzyloxy)-6-bromonaphthalen-2-amine (3)

3-(Benzyloxy)-1,6-dibromonaphthalen-2-amine (180 g, 80% purity, 354 mmol) was taken up in EtOH (1500 mL) and treated with tin metal (63 g, 531 mmol). To the slurry was then added 37% HCl (500 mL) and the mixture heated to 90° C. for 1 h. The reaction was cooled to RT filtered and the filtrate poured into water (500 mL). The mixture was adjusted to pH 8 with solid NaHCO₃ and extracted with ethyl acetate (3×300 mL). The combined organic phases were washed with brine (2×100 mL), dried over Na₂SO₄, filtered and concentrated under reduced pressure to give 3-(benzyloxy)-6-bromonaphthalen-2-amine (100 g, 274 mmol, 78% yield, 90% purity) as a khaki solid.

LCMS (TFA, ESI+): m/z 328.2 [M+H]⁺

¹H NMR (400 MHZ, DMSO-d₆) δ 5.15-5.36 (m, 4H), 6.93 (s, 1H), 7.24-7.30 (m, 2H), 7.32-7.38 (m, 1H), 7.38-7.48 (m, 3H), 7.56 (d, J=7.3 Hz, 2H), 7.80 (d, J=1.6 Hz, 1H).

### Step 3: 3-(Benzyloxy)-6-bromo-1-fluoronaphthalen-2-amine (4)

To a solution of 3-(benzyloxy)-6-bromonaphthalen-2-amine (100 g, 95% purity, 289 mmol) in THF (1500 mL) was added N-fluorobenzenesulfonimide (100 g, 318 mmol) at RT. After 12 h, the mixture was quenched with saturated aqueous sodium thiosulfate (500 mL) and extracted with ethyl acetate (3×500 mL). The organic layers were combined, washed with brine (400 mL); then dried (Na₂SO₄), filtered and concentrated. The oil was purified by silica gel chromatography (0% to 5% ethyl acetate:petroleum ether) to afford 3-(benzyloxy)-6-bromo-1-fluoronaphthalen-2-amine (58 g, 142 mmol, 49% yield, 85% purity) as yellow solid.

LCMS (TFA, ESI+): m/z 345.9 [M+H]⁺

¹H NMR (400 MHZ, DMSO-d₆) δ 5.22-5.32 (m, 4H) 7.23-7.26 (m, 1H) 7.33-7.38 (m, 1H) 7.40-7.45 (m, 3H) 7.55-7.60 (m, 2H) 7.62-7.67 (m, 1H) 7.91-7.95 (m, 1H).

### Step 4: Methyl 2-((3-(benzyloxy)-6-bromo-1-fluoronaphthalen-2-yl)amino)acetate (5)

To a solution of 3-(benzyloxy)-6-bromo-1-fluoronaphthalen-2-amine (7.5 g, 82% purity, 17.8 mmol) in DMF (70 mL) at RT was added N,N-diisopropylethylamine (12.4 mL, 71.1 mmol) and methyl 2-bromoacetate (16.3 g, 107 mmol) This mixture was then heated to 65° C. for 12 h. The reaction was cooled to RT and poured into water (100 mL) then extracted with ethyl acetate (3×80 mL). The organic phases were washed with brine (50 mL); then dried with Na₂SO₄, filtered and concentrated. The crude product was triturated with 80% petroleum ether:ethyl acetate (20 mL) and the solid product filtered and dried to give methyl 2-((3-(benzyloxy)-6-bromo-1-fluoronaphthalen-2-yl)amino)acetate (5 g, 10 mmol, 50% yield: 80% purity) as brown solid.

LCMS (TFA, ESI+): m/z 418.1 [M+H]⁺

¹H NMR (400 MHZ, DMSO-d₆) δ 3.63 (s, 3H), 4.21 (dd, J=6.5, 3.9 Hz, 2H), 5.29 (s, 2H), 5.57-5.63 (m, 1H), 7.28 (s, 1H), 7.34-7.45 (m, 4H), 7.55 (d, J=7.3 Hz, 2H), 7.63 (d, J=8.8 Hz, 1H), 7.94 (s, 1H)

### Step 5: Methyl 2-((3-(benzyloxy)-6-bromo-1-fluoronaphthalen-2-yl)(N-(tert-butoxycarbonyl)sulfamoyl)amino)acetate (6)

To a solution of sulfurisocyanatidic chloride (18.3 g, 129 mmol) in CH₂Cl₂ (60 mL) at 0° C. was added t-BuOH (12.4 mL, 129 mmol) in CH₂Cl₂ (30 mL); then warmed to RT and stirring continued for 1 h. The reaction was recooled to 0° C.) and treated with a solution of triethylamine (36 mL, 260 mmol) and methyl 2-((3-(benzyloxy)-6-bromo-1-fluoronaphthalen-2-yl)amino)acetate (30 g, 90% purity, 65 mmol) as a solution in CH₂Cl₂ (90 mL). The cold bath was removed and stirring continued at RT for 2 h. The reaction was concentrated to remove most of the solvent and the crude methyl 2-((3-(benzyloxy)-6-bromo-1-fluoronaphthalen-2-yl)(N-(tert-butoxy carbonyl)sulfamoyl)amino)acetate (75 g, 96% yield: 45% purity) was used directly in the next step as yellow oil.

LCMS (TFA, ESI+): m/z 497.1 [M-Boc]⁺

¹H NMR (400 MHZ, DMSO-d₆) δ 1.30 (s, 9H), 3.53 (s, 3H), 4.45 (d, J=18.0 Hz, 1H), 4.76 (d, J=18.0 Hz, 1H), 5.17-5.35 (m, 2H), 7.31-7.37 (m, 2H), 7.38-7.44 (m, 2H), 7.52-7.62 (m, 3H), 7.92 (d, J=8.8 Hz, 1H), 8.12 (s, 1H)

### Step 6: Methyl 2-((3-(benzyloxy)-6-bromo-1-fluoronaphthalen-2-yl)(sulfamoyl)amino)acetate (7)

A crude sample of methyl 2-((3-(benzyloxy)-6-bromo-1-fluoronaphthalen-2-yl)(N-(tert-butoxy carbonyl)sulfamoyl)amino)acetate (130 g, 50% purity, 109 mmol) in CH₂Cl₂ (700 mL) was treated with TFA (250 mL, 3.3 mol) at 0° C. After 1 h at RT, the reaction was concentrated and the residue treated with saturated sodium bicarbonate solution. The aqueous mixture was extracted with ethyl acetate (3×1 L) and the organic phases combined, washed with brine (1 L); then dried with Na₂SO₄, filtered and concentrated under reduced pressure to provide methyl 2-((3-(benzyloxy)-6-bromo-1-fluoronaphthalen-2-yl)(sulfamoyl)amino)acetate (46 g, 83 mmol, 77% yield, 90% purity) as a white solid.

LCMS (TFA, ESI+): m/z 497.1 [M+H]⁺

¹H NMR (400 MHZ, DMSO-d₆) δ 3.56 (s, 3H), 4.29-4.34 (m, 1H), 4.44-4.53 (m, 1H), 5.26 (s, 2H), 7.11 (s, 2H), 7.33-7.44 (m, 5H), 7.58 (d, J=7.5 Hz, 3H), 7.92 (d, J=8.9 Hz, 1H), 8.14 (s, 1H).

### Step 7: 5-(3-(Benzyloxy)-6-bromo-1-fluoronaphthalen-2-yl)-1,2,5-thiadiazolidin-3-one 1,1-dioxide (8)

To a solution of methyl 2-((3-(benzyloxy)-6-bromo-1-fluoronaphthalen-2-yl)(sulfamoyl)amino)acetate (54 g, 90% purity, 98 mmol) in THF (500 mL) at RT was added 30% sodium methoxide in methanol (148 mL, 42 g, 146 mmol). After 15 min, the reaction was quenched with 150 mL of 1 M HCl and extracted with ethyl acetate (3×300 mL). The combined organic phases were washed with brine (200 mL), dried with Na₂SO₄, filtered and concentrated under reduced pressure to give 5-(3-(benzyloxy)-6-bromo-1-fluoronaphthalen-2-yl)-1,2,5-thiadiazolidin-3-one 1,1-dioxide (27 g, 52 mmol, 63% yield, 90% purity) as yellow solid.

LCMS (NH₄HCO₃, ESI−): m/z 463.0 [M−H]⁻

¹H NMR (400 MHZ, DMSO-d₆) δ 4.27 (s, 2H), 5.26 (s, 2H), 7.26-7.46 (m, 4H), 7.51-7.64 (m, 3H), 7.91 (d, J=8.9 Hz, 1H), 8.15 (s, 1H).

### 5-(6-Amino-1-fluoro-3-hydroxynaphthalen-2-yl)-1,2,5-thiadiazolidin-3-one 1,1-dioxide, ammonium salt (6)

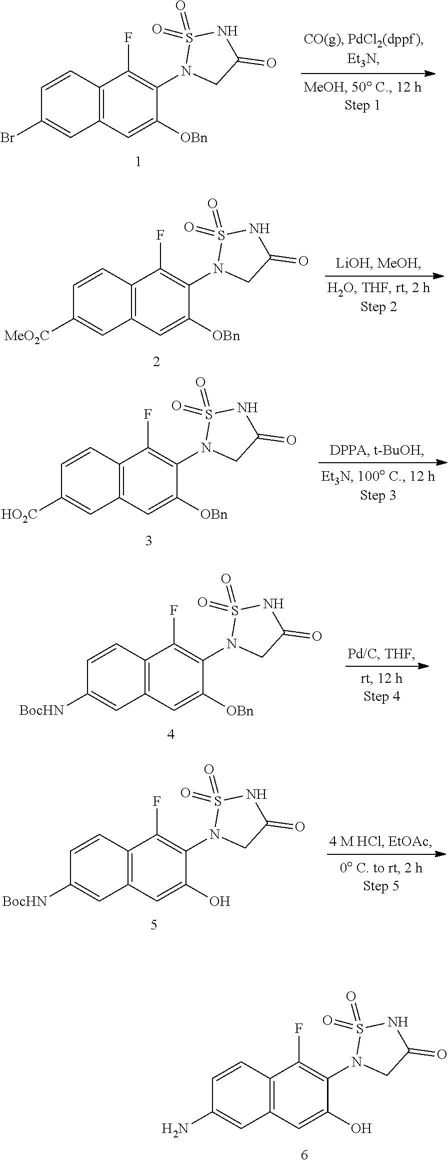 [SMILES: O=C1CN(c2c(OCc3ccccc3)cc3cc(Br)ccc3c2F)S(=O)(=O)N1 ; COC(=O)c1ccc2c(F)c(N3CC(=O)NS3(=O)=O)c(OCc3ccccc3)cc2c1 ; O=C1CN(c2c(OCc3ccccc3)cc3cc(C(=O)O)ccc3c2F)S(=O)(=O)N1 ; CC(C)(C)OC(=O)Nc1ccc2c(F)c(N3CC(=O)NS3(=O)=O)c(OCc3ccccc3)cc2c1 ; CC(C)(C)OC(=O)Nc1ccc2c(F)c(N3CC(=O)NS3(=O)=O)c(O)cc2c1 ; Nc1ccc2c(F)c(N3CC(=O)NS3(=O)=O)c(O)cc2c1]

### Step 1: Methyl 7-(benzyloxy)-6-(1,1-dioxido-4-oxo-1,2,5-thiadiazolidin-2-yl)-5-fluoro-2-naphthoate (2)

To a solution of 5-(3-(benzyloxy)-6-bromo-1-fluoronaphthalen-2-yl)-1,2,5-thiadiazolidin-3-one 1,1-dioxide (40 g, 90% purity, 77 mmol) in MeOH (400 mL) was added triethylamine (32.4 mL, 232 mmol) and [1,1′-bis(diphenylphosphino)ferrocene]dichloropalladium(II) (17.0 g, 23.2 mmol) and the reaction placed under an atmosphere of carbon monoxide (40 psi). The solution was heated to 50° C. and stirred for 12 h. Upon consumption of starting material, the solvent was removed under reduced pressure to give methyl 7-(benzyloxy)-6-(1,1-dioxido-4-oxo-1,2,5-thiadiazolidin-2-yl)-5-fluoro-2-naphthoate (70 g, 95 mmol, 81% yield: 60% purity) as red solid which was used directly in the next step. LCMS (NH₄HCO₃, ESI−): m/z 443.1 [M−H]^{- 1}H NMR (400 MHZ, DMSO-d₆) δ 3.92 (s, 3H), 3.97-4.27 (m, 2H), 5.28 (s, 2H), 7.26-7.41 (m, 3H), 7.45-7.68 (m, 3H), 7.78 (s, 1H), 7.84-8.02 (m, 1H), 8.23 (d, J=8.8 Hz, 1H), 8.55 (d, J=1.3 Hz, 1H).

### Step 2: 7-(Benzyloxy)-6-(1,1-dioxido-4-oxo-1,2,5-thiadiazolidin-2-yl)-5-fluoro-2-naphthoic acid (3)

To a solution of methyl 7-(benzyloxy)-6-(1,1-dioxido-4-oxo-1,2,5-thiadiazolidin-2-yl)-5-fluoro-2-naphthoate (8 g, 80% purity, 14.4 mmol) in THF (20 mL), MeOH (5 mL) and water (5 mL) was added LiOH (0.345 g, 14.4 mmol) at 0° C. The mixture was warmed to RT and stirred for 2 h. The reaction was concentrated to remove most of the THF then diluted with water (100 mL). The aqueous phase was washed with ethyl acetate (3×100 mL) then acidified with 1 M hydrochloride acid to pH=2. The aqueous solution was extracted with ethyl acetate (3×150 mL) and the organic layers were combined and washed with brine (150 mL); then dried with Na₂SO₄, filtered and concentrated under reduced pressure to give 7-(benzyloxy)-6-(1,1-dioxido-4-oxo-1,2,5-thiadiazolidin-2-yl)-5-fluoro-2-naphthoic acid (6.8 g, 13 mmol, 88% yield, 80% purity) as a yellow solid. LCMS (NH₄HCO₃, ESI−): m/z 429.1 [M−H]^{- 1}H NMR (400 MHZ, DMSO-d₆) δ 4.55 (s, 2H), 5.30 (s, 2H), 7.30-7.43 (m, 3H), 7.49-7.58 (m, 2H), 7.71 (s, 1H), 7.94 (dd, J=8.7, 1.4 Hz, 1H), 8.04-8.14 (m, 1H), 8.55 (s, 1H).

### Step 3: tert-Butyl (7-(benzyloxy)-6-(1,1-dioxido-4-oxo-1,2,5-thiadiazolidin-2-yl)-5-fluoronaphthalen-2-yl)carbamate (4)

To a solution of 7-(benzyloxy)-6-(1,1-dioxido-4-oxo-1,2,5-thiadiazolidin-2-yl)-5-fluoro-2-naphthoic acid (1.3 g, 93% purity, 2.8 mmol) in t-BuOH (50 mL) at RT was added triethylamine (0.78 mL, 5.6 mmol) and diphenylphosphoryl azide (1.14 g, 4.17 mmol). The reaction was heated to 100° C. and stirred for 12 h. The solution was concentrated under reduced pressure and diluted with water (50 mL). The aqueous mixture was extracted with ethyl acetate (3×30 mL); and the combined organic phases were washed with brine (30 mL), dried with Na₂SO₄, filtered, and concentrated under reduced pressure to give tert-butyl (7-(benzyloxy)-6-(1,1-dioxido-4-oxo-1,2,5-thiadiazolidin-2-yl)-5-fluoronaphthalen-2-yl)carbamate (1.5 g, 1.9 mmol, 70.0% yield, 65% purity) as an off-white solid. LCMS (NH₄HCO₃, ESI−): m/z 500.2 [M−H]^{- 1}H NMR (400 MHZ, DMSO-d₆) δ 1.47 (s, 9H), 4.30 (s, 2H), 5.22 (s, 2H), 7.18 (s, 1H), 7.28-7.42 (m, 4H), 7.49 (d, J=7.5 Hz, 2H), 7.84 (d, J=8.8 Hz, 1H), 8.00 (s, 1H).

### Step 4: tert-Butyl (6-(1,1-dioxido-4-oxo-1,2,5-thiadiazolidin-2-yl)-5-fluoro-7-hydroxynaphthalen-2-yl)carbamate (5)

To a solution of tert-butyl (7-(benzyloxy)-6-(1,1-dioxido-4-oxo-1,2,5-thiadiazolidin-2-yl)-5-fluoronaphthalen-2-yl)carbamate (0.9 g, 90% purity, 1.6 mmol) in THF (10 mL) was added Pd/C (17 mg, 0.16 mmol) at RT. Stirring was continued for 12 h under a hydrogen atmosphere (15 psi). The resulting suspension was filtered through a pad of Celite and the pad washed with MeOH (75 mL). The combined filtrates were concentrated to dryness to give tert-butyl (6-(1,1-dioxido-4-oxo-1,2,5-thiadiazolidin-2-yl)-5-fluoro-7-hydroxynaphthalen-2-yl)carbamate (0.8 g, 1.6 mmol, 96% yield, 80% purity) as white solid which was used directly in the next step. LCMS (NH₄HCO₃, ESI−): m/z 410.1 [M−H]^{- 1}H NMR (400 MHZ, DMSO-d₆) δ 1.50 (s, 9H), 4.06 (s, 2H), 6.90 (s, 1H), 7.35 (dd, J=9.1, 1.8 Hz, 1H), 7.76 (d, J=8.9 Hz, 1H), 7.91 (s, 1H), 9.56-9.70 (m, 2H).

### Step 5: 5-(6-Amino-1-fluoro-3-hydroxynaphthalen-2-yl)-1,2,5-thiadiazolidin-3-one 1,1-dioxide, ammonium salt (6)

A solution of tert-butyl (6-(1,1-dioxido-4-oxo-1,2,5-thiadiazolidin-2-yl)-5-fluoro-7-hydroxynaphthalen-2-yl)carbamate (5 g, 90% purity, 11 mmol) in ethyl acetate (30 mL) was treated with 4 M HCl (2.7 mL, 11 mmol) at 0° C. Upon completion of addition, the mixture was warmed to RT, and stirring was continued for 2 h. The solution was concentrated under reduced pressure to give a crude product which was purified by preparative HPLC The column used for chromatography was [column: Xbridge Shield RP18, 2.1×50 mm, 5 μm particles; detection; DAD; MS: negative electrospray ionization, range: 100-1000; mobile phase: A: 10 mM ammonium bicarbonate_{(aq)}; mobile phase B: acetonitrile; gradient: 5-95% B in 2.05 min, 5% B in 0.01 min, 5-95% B (0.01-1.00 min), 95-100% B (1.00-1.80 min), 5% B in 1.81 min with a hold at 5% B for 0.24 min; and flowrate: 1.0 mL/min]. The appropriate fractions were collected, and the sample was lyophilized to give 5-(6-amino-1-fluoro-3-hydroxynaphthalen-2-yl)-1,2,5-thiadiazolidin-3-one 1,1-dioxide, ammonium salt (3.28 g, 9.49 mmol, 87% yield, 95% purity) as an off white solid. LCMS (NH₄HCO₃, ESI−): m/z 310.0 [M−H]^{- 1}H NMR (400 MHZ, DMSO-d₆) δ 4.05 (s, 2H), 6.63 (d, J=13.6 Hz, 2H), 6.77 (dd, J=8.9, 2.0 Hz, 1H), 6.97 (s, 1H), 7.10 (s, 1H), 7.22 (s, 1H), 7.57 (d, J=8.9 Hz, 1H), 9.29 (br s, 1H).

### 5-(6-amino-3-(benzyloxy)-1-fluoronaphthalen-2-yl)-1,2,5-thiadiazolidin-3-one 1,1-dioxide (2)

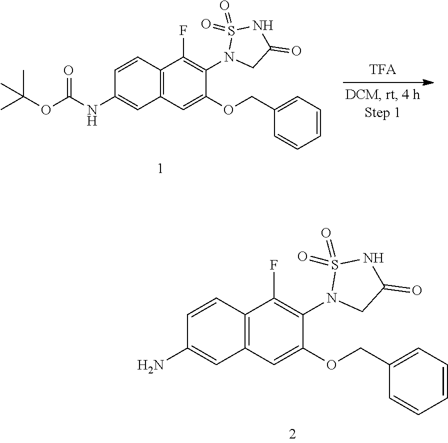 [SMILES: CC(C)(C)OC(=O)Nc1ccc2c(F)c(N3CC(=O)NS3(=O)=O)c(OCc3ccccc3)cc2c1 ; Nc1ccc2c(F)c(N3CC(=O)NS3(=O)=O)c(OCc3ccccc3)cc2c1]

### Step 1: 5-(6-amino-3-(benzyloxy)-1-fluoronaphthalen-2-yl)-1,2,5-thiadiazolidin-3-one 1,1-dioxide (2)

A stirred solution of tert-butyl N-[7-benzyloxy-5-fluoro-6-(1,1,4-trioxo-1,2,5-thiadiazolidin-2-yl)-2-naphthyl]carbamate (1, 1.0 g, 1.99 mmol) in DCM (10 mL) at 0° C. was treated with trifluoroacetic acid (227.35 mg, 1.99 mmol, 153.62 μL) via dropwise addition. The reaction mixture was stirred at RT for 4 h. The reaction mixture was concentrated under reduced pressure, azeotroped with toluene, and triturated with diethyl ether to obtain 5-(6-amino-3-benzyloxy-1-fluoro-2-naphthyl)-1,1-dioxo-1,2,5-thiadiazolidin-3-one (2, 850 mg, 1.58 mmol, 79.44% yield, TFA salt) as an off-white solid. LCMS (ES+): m/z 402.1 [M+H]⁺

### 5-(3-(Benzyloxy)-7-bromo-1-fluoronaphthalen-2-yl)-1,2,5-thiadiazolidin-3-one 1,1-dioxide (12)

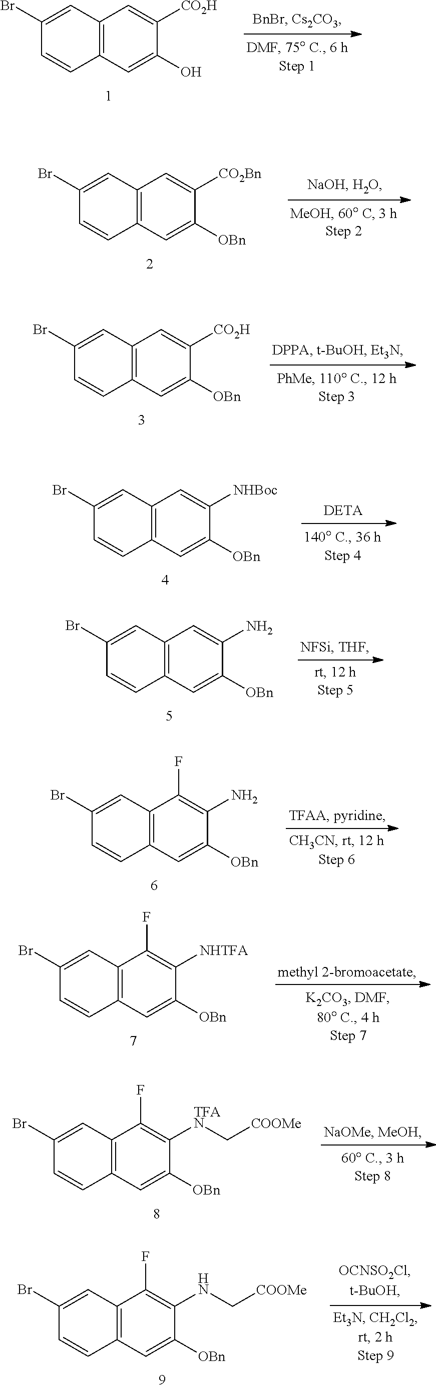

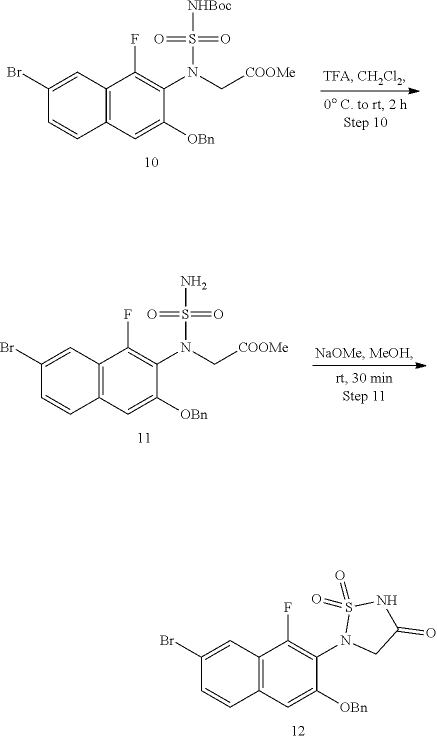 [SMILES: COC(=O)CN(c1c(OCc2ccccc2)cc2ccc(Br)cc2c1F)S(=O)(=O)NC(=O)OC(C)(C)C ; COC(=O)CN(c1c(OCc2ccccc2)cc2ccc(Br)cc2c1F)S(N)(=O)=O ; O=C1CN(c2c(OCc3ccccc3)cc3ccc(Br)cc3c2F)S(=O)(=O)N1]

### Step 1: Benzyl 3-(benzyloxy)-7-bromo-2-naphthoate (2)

A 100 mL round-bottom flask was charged with 7-bromo-3-hydroxy-2-naphthoic acid ([1779-11-9], 5 g, 18.7 mmol) and cesium carbonate (18.30 g, 56.2 mmol), followed by DMF (35 mL). The mixture was rapidly stirred to suspend the reaction components, followed by treatment with benzyl bromide (4.45 mL, 37.4 mmol) at RT. After 2 h, the mixture was poured into water (70 mL), and the resulting white solid precipitate collected by filtration. The solid thus obtained was washed with water (3×50 mL), triturated with 30% methyl tert-butylmethyl ether/petroleum ether (20 mL), filtered, and dried under vacuum to afford benzyl 3-(benzyloxy)-7-bromo-2-naphthoate (8 g, 17.2 mmol, 92% yield, 96% purity) as a white solid. LCMS (TFA, ESI+): m/z 447.1 [M+H]^{+ 1}H NMR (400 MHZ, DMSO-d₆) δ 5.27 (s, 2H), 5.35 (s, 2H), 7.30-7.45 (m, 8H), 7.49 (d, J=6.8 Hz, 2H), 7.60-7.71 (m, 2H), 7.82 (d, J=8.8 Hz, 1H), 8.28 (d, J=1.5 Hz, 1H), 8.32 (s, 1H).

### Step 2: 3-(Benzyloxy)-7-bromo-2-naphthoic acid (3)

To a solution of benzyl 3-(benzyloxy)-7-bromo-2-naphthoate (4 g, 8.5 mmol) in MeOH (60 mL) and water (30.0 mL) at RT was added LiOH (0.407 g, 17.0 mmol). The mixture was heated to 70° C. for 2 h and was then concentrated. The resulting residue was diluted with water (500 mL). The aqueous layer was acidified with 1 M HCl to pH=3, and the solid was filtered and dried under vacuum to give 3-(benzyloxy)-7-bromo-2-naphthoic acid (3 g, 8.0 mmol, 94% yield, 95% purity) as white solid. LCMS (TFA, ESI+): m/z 357.0 [M+H]^{+ 1}H NMR (400 MHZ, DMSO-d₆) δ 5.29 (s, 2H), 7.29-7.45 (m, 3H), 7.54 (d, J=7.28 Hz, 2H), 7.60 (s, 1H), 7.66 (dd, J=8.8, 2.0 Hz, 1H), 7.81 (d, J=8.8 Hz, 1H), 8.20-8.27 (m, 2H), 13.06 (br s, 1H).

### Step 3: tert-Butyl (3-(benzyloxy)-7-bromonaphthalen-2-yl)carbamate (4)

A three-necked 250 mL round bottom flask was charged with 3-(benzyloxy)-7-bromo-2-naphthoic acid (6 g, 16.8 mmol), toluene (48 mL), 1-BuOH (48 mL) and triethylamine (2.48 mL, 17.8 mmol). Diphenyl phosphorazidate (4.90 g, 17.8 mmol) was then added and the reaction mixture heated at 110° C. for 4 h. The solution was cooled to RT and concentrated to give a crude solid. The solid was triturated with ethanol (50 mL), filtered, rinsed with ethanol (10 mL), and dried under vacuum to give tert-butyl (3-(benzyloxy)-7-bromonaphthalen-2-yl)carbamate (6.6 g, 13.9 mmol, 83% yield, 90% purity) as white solid. LCMS (NH₄HCO₃, ESI−): m/z 426.1 [M−H]^{- 1}H NMR (400 MHz, DMSO-d₆) δ 1.48 (s, 9H), 5.29 (s, 2H), 7.34-7.50 (m, 5H), 7.57 (d, J=7.0 Hz, 2H), 7.68 (d, J=8.8 Hz, 1H), 8.02 (d, J=1.7 Hz, 1H), 8.13 (s, 1H), 8.21 (s, 1H).

### Step 4: 3-(Benzyloxy)-7-bromonaphthalen-2-amine (5)

To a solution of tert-butyl (3-(benzyloxy)-7-bromonaphthalen-2-yl)carbamate (8 g, 86% purity, 16 mmol) was added diethylenetriamine (26.2 g, 254 mmol) and the mixture was stirred at 130° C. for 3 h. The reaction was cooled to RT, and water (50 mL) was added to the mixture and stirred 10 min. The solid was filtered and the filter cake was washed with 10 mL of i-PrOH and dried under vacuum to give 3-(benzyloxy)-7-bromonaphthalen-2-amine (4.5 g, 12.3 mmol, 78% yield, 90% purity) as pink solid. LCMS (TFA, ESI+): m/z 328.1 [M+H]^{+ 1}H NMR (400 MHZ, DMSO-d₆) δ 5.25 (s, 2H), 5.37 (s, 2H), 6.89 (s, 1H), 7.18 (dd, J=8.6, 2.0 Hz, 1H), 7.27-7.37 (m, 2H), 7.38-7.45 (m, 2H), 7.54 (t, J=7.7 Hz, 3H), 7.70 (d, J=1.7 Hz, 1H).

### Step 5: 3-(Benzyloxy)-7-bromo-1-fluoronaphthalen-2-amine (6)

To a solution of 3-(benzyloxy)-7-bromonaphthalen-2-amine (20 g, 90% purity, 54.8 mmol) in THF (100 mL) was added a solution of N-fluorobenzenesulfonimide (19.0 g, 60.3 mmol) in THF (100 mL) at (° C.) over the period of 1 h. The mixture was warmed to RT and stirred for an additional 1 h. Then residual oxidant was quenched by adding a solution of sodium thiosulfate pentahydrate (17.3 g, 110 mmol) in water (100 mL), and the mixture stirred at RT for 20 min. The aqueous phase was extracted with ethyl acetate (3×100 mL) and the combined organic phases washed with brine (2×100 mL); then dried over Na₂SO₄, filtered and concentrated under reduced pressure. The residue was purified by silica gel chromatography (0)% to 10% ethyl acetate:petroleum ether) to give 3-(benzyloxy)-7-bromo-1-fluoronaphthalen-2-amine (8 g, 20.8 mmol, 38% yield, 90% purity) as yellow solid. LCMS (TFA, ESI+): m/z 346.2 [M+H]^{+ 1}H NMR (400 MHZ, DMSO-d₆) δ 5.28 (s, 2H), 5.31 (s, 2H), 7.25 (s, 1H), 7.30-7.36 (m, 2H), 7.39-7.44 (m, 2H), 7.56 (br d, J=7.1 Hz, 2H), 7.65 (dd, J=8.6, 1.3 Hz, 1H), 7.82 (d, J=1.6 Hz, 1H).

### Step 6: N-(3-(Benzyloxy)-7-bromo-1-fluoronaphthalen-2-yl)-2,2,2-trifluoroacetamide (7)

To a solution of 3-(benzyloxy)-7-bromo-1-fluoronaphthalen-2-amine (2 g, 90% purity, 5.2 mmol) in acetonitrile (40 mL) and pyridine (1.3 mL, 15.6 mmol) at (° C.) was added trifluoroacetic anhydride (1.49 mL, 10.4 mmol), and the mixture allowed to warm slowly to RT. After 2 h. the mixture was diluted with water (20 mL) and extracted with ethyl acetate (3×20 mL). The organic layers were washed with brine (20 mL), dried with Na₂SO₄, filtered, and concentrated under reduced pressure to give N-(3-(benzyloxy)-7-bromo-1-fluoronaphthalen-2-yl)-2,2,2-trifluoroacetamide (2.5 g, 4.8 mmol, 92% yield, 85% purity) as an off-white solid which was used in the next step directly. LCMS (TFA, ESI+): m/z 442.0 [M+H]^{+ 1}H NMR (400 MHZ, DMSO-d₆) δ 5.28 (s, 2H), 7.30-7.35 (m, 1H), 7.39 (t, J=7.3 Hz, 2H), 7.46 (br d, J=7.0 Hz, 2H), 7.53 (s, 1H), 7.70-7.75 (m, 1H), 7.88 (d, J=8.4 Hz, 1H), 8.15 (s, 1H).

### Step 7: Methyl 2-(N-(3-(benzyloxy)-7-bromo-1-fluoronaphthalen-2-yl)-2,2,2-trifluoroacetamido)acetate (8)

To a solution of N-(3-(benzyloxy)-7-bromo-1-fluoronaphthalen-2-yl)-2,2,2-trifluoroacetamide (2.5 g, 85% purity, 4.81 mmol) in DMF (30 mL) was added K₂CO₃ (1.33 g, 9.61 mmol) and methyl 2-bromoacetate (1.10 g, 7.21 mmol). The reaction was heated to 80° C. and stirred for 1 h. The mixture was cooled to RT and diluted with water (30 mL). The aqueous mixture was extracted with ethyl acetate (3×20 mL), and the combined organic phases were washed with brine (3×20 mL), dried with Na₂SO₄, filtered and concentrated under reduced pressure to give methyl 2-(N-(3-(benzyloxy)-7-bromo-1-fluoronaphthalen-2-yl)-2,2,2-trifluoroacetamido)acetate (3.4 g, 5.95 mmol, 93% yield, 90% purity) as an off-white solid which was used in next step directly. LCMS (TFA, ESI+): m/z 514.0 [M+H]^{+ 1}H NMR (400 MHZ, DMSO-d₆) δ 8.27 (d, J=2.0 Hz, 1H), 8.11 (d, J=9.0 Hz, 1H), 7.78-7.66 (m, 2H), 7.51-7.33 (m, 5H), 5.27 (q, J=11.9 Hz, 2H), 4.45 (d, J=1.7 Hz, 2H), 3.59 (s, 3H).

### Step 8: Methyl 2-((3-(benzyloxy)-7-bromo-1-fluoronaphthalen-2-yl)amino)acetate (9)

To a solution of methyl 2-(N-(3-(benzyloxy)-7-bromo-1-fluoronaphthalen-2-yl)-2,2,2-trifluoroacetamido)acetate (3.4 g, 85% purity, 5.6 mmol) in MeOH (40 mL) was added sodium methoxide (4.29 g, 23.8 mmol) at RT. The mixture was heated to 60° C. and stirred for 3 h. Upon completion, the mixture was cooled to RT, diluted with water (30 mL), and the aqueous mixture extracted with ethyl acetate (3×20 mL). The combined organic phases were washed with brine (2×20) mL), dried with Na₂SO₄, filtered, and concentrated under reduced pressure to give methyl 2-((3-(benzyloxy)-7-bromo-1-fluoronaphthalen-2-yl)amino)acetate (1.9 g, 4.1 mmol, 69% yield. 90% purity) as an off-white solid which was used directly in the next step. LCMS (TFA, ESI+): m/z 418.2 [M+H]^{+ 1}H NMR (400 MHZ, DMSO-d₆) δ 3.63 (s, 3H), 4.22 (dd, J=6.7, 4.0 Hz, 2H), 5.30 (s, 2H), 7.30 (s, 1H), 7.34-7.39 (m, 2H), 7.41-7.45 (m, 2H), 7.55 (d, J=7.1 Hz, 2H), 7.67 (dd, J=8.7, 1.5 Hz, 1H), 7.80 (d, J=1.7 Hz, 1H).

### Step 9: Methyl 2-((3-(benzyloxy)-7-bromo-1-fluoronaphthalen-2-yl)(N-(tert-butoxycarbonyl)sulfamoyl)amino)acetate (10)

To a solution of sulfurisocyanatidic chloride (1.22 g, 8.61 mmol) in CH₂Cl₂ (10 mL) was added a solution of 1-BuOH (1.30 g, 17.5 mmol) in CH₂Cl₂ (10 mL), dropwise, at (° C.) The mixture was warmed to RT and stirred for an additional 1 h. After cooling to 0° C., a solution of triethylamine (2.40) mL, 17.2 mmol) and methyl 2-((3-(benzyloxy)-7-bromo-1-fluoronaphthalen-2-yl)amino)acetate (2 g, 90% purity, 4.30 mmol) in CH₂Cl₂ (20 mL) was slowly added to the reaction mixture. Upon complete addition, the solution was warmed to RT and stirred for 2 h. The mixture was concentrated under pressure to give methyl 2-((3-(benzyloxy)-7-bromo-1-fluoronaphthalen-2-yl)(N-(tert-butoxy carbonyl)sulfamoyl)amino)acetate (5 g, 6.70 mmol, 89% yield, 80% purity) as yellow oil. The crude product was used for the next step without purification. LCMS (TFA, ESI+): m/z 497.2 [M-Boc+H]^{+ 1}H NMR (400 MHZ, DMSO-d₆) δ 11.40 (s, 1H), 8.15 (d, J=2.0 Hz, 1H), 7.83 (dd, J=8.9, 1.3 Hz, 1H), 7.71 (dd, J=8.9, 2.0 Hz, 1H), 7.60-7.48 (m, 2H), 7.47-7.30 (m, 4H), 5.31 (q, J=12.8 Hz, 2H), 4.75 (d, J=17.9 Hz, 1H), 4.48 (d, J=17.9 Hz, 1H), 3.56 (s, 3H), 1.32 (s, 9H).

### Step 10: Methyl 2-((3-(benzyloxy)-7-bromo-1-fluoronaphthalen-2-yl)(sulfamoyl)amino)acetate (11)

To a solution of methyl 2-((3-(benzyloxy)-7-bromo-1-fluoronaphthalen-2-yl)(N-(tert-butoxy carbonyl)sulfamoyl)amino)acetate (15 g, 75% purity, 18.8 mmol) in CH₂Cl₂ (100 mL) at 0° C.) was added 2,2,2-trifluoroacetic acid (35 mL, 18.8 mmol), then warmed to RT and stirred for 1 h. The mixture was concentrated under reduced pressure, and the residue diluted with water (300 mL). The aqueous mixture was made basic by addition of solid NaHCO₃(pH=8) and then extracted with ethyl acetate (3×150 mL). The combined organic layers were dried over Na₂SO₄, filtered, and concentrated under reduced pressure to give methyl 2-((3-(benzyloxy)-7-bromo-1-fluoronaphthalen-2-yl)(sulfamoyl)amino)acetate (12 g, 16.9 mmol, 90% yield, 70% purity) as white solid which was used for next step without further purification. LCMS (TFA, ESI+): m/z 496.9 [M+H]^{+ 1}H NMR (400 MHZ, DMSO-d₆) δ 3.56 (s, 3H), 4.29-4.36 (m, 1H), 4.46-4.53 (m, 1H), 5.27 (s, 2H), 7.11 (s, 2H), 7.39-7.46 (m, 4H), 7.58 (d, J=7.2 Hz, 2H), 7.69 (dd, J=8.8, 2.0 Hz, 1H), 7.81-7.86 (m, 1H), 8.13 (d, J=2.0 Hz, 1H).

### Step 11: 5-(3-(Benzyloxy)-7-bromo-1-fluoronaphthalen-2-yl)-1,2,5-thiadiazolidin-3-one 1,1-dioxide (12)

To a solution of methyl 2-((3-(benzyloxy)-7-bromo-1-fluoronaphthalen-2-yl)(sulfamoyl)amino)acetate (9 g, 85% purity, 15.4 mmol) in THF (100 mL) at RT was added solution of 30% sodium methoxide in methanol (29.3 mL, 8.31 g, 46.1 mmol) and stirring was continued for 1 h. The reaction was concentrated, taken up in water (10 mL), and acidified with 1 M HCl (pH=5). The aqueous mixture was extracted with ethyl acetate (3×20 mL). The combined organic phases were dried over anhydrous Na₂SO₄, filtered, and concentrated to give 5-(3-(benzyloxy)-7-bromo-1-fluoronaphthalen-2-yl)-1,2,5-thiadiazolidin-3-one 1,1-dioxide (9 g, 17.4 mmol, 90% yield, 90% purity) as light brown solid. LCMS (NH₄HCO₃, ESI−): m/z 463.0 [M−H]^{- 1}H NMR (400 MHZ, DMSO-d₆) δ 4.53 (s, 2H), 5.28 (s, 2H), 7.30-7.43 (m, 4H), 7.52 (br d, J=7.6 Hz, 3H), 7.74 (dd, J=8.8, 1.8 Hz, 1H), 7.87 (d, J=8.8 Hz, 1H), 8.16 (d, J=1.4 Hz, 1H).

### 5-(7-(2-Aminoethoxy)-1-fluoro-3-hydroxynaphthalen-2-yl)-1,2,5-thiadiazolidin-3-one 1,1-dioxide (6)

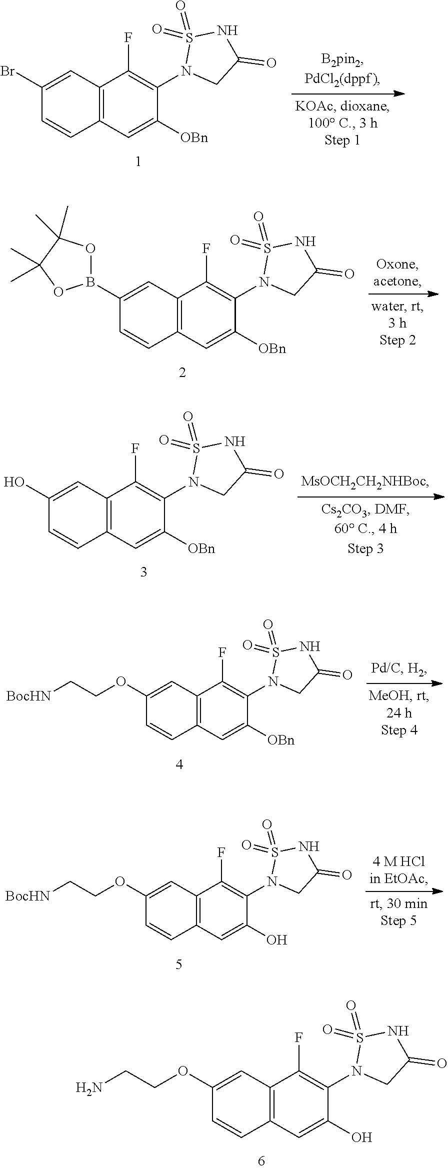 [SMILES: O=C1CN(c2c(OCc3ccccc3)cc3ccc(Br)cc3c2F)S(=O)(=O)N1 ; CC1(C)OB(c2ccc3cc(OCc4ccccc4)c(N4CC(=O)NS4(=O)=O)c(F)c3c2)OC1(C)C ; O=C1CN(c2c(OCc3ccccc3)cc3ccc(O)cc3c2F)S(=O)(=O)N1 ; CC(C)(C)OC(=O)NCCOc1ccc2cc(OCc3ccccc3)c(N3CC(=O)NS3(=O)=O)c(F)c2c1 ; CC(C)(C)OC(=O)NCCOc1ccc2cc(O)c(N3CC(=O)NS3(=O)=O)c(F)c2c1 ; NCCOc1ccc2cc(O)c(N3CC(=O)NS3(=O)=O)c(F)c2c1]

### Step 1: 5-(3-(Benzyloxy)-1-fluoro-7-(4,4,5,5-tetramethyl-1,3,2-dioxaborolan-2-yl)naphthalen-2-yl)-1,2,5-thiadiazolidin-3-one 1,1-dioxide (2)

To a solution of 5-(3-(benzyloxy)-7-bromo-1-fluoronaphthalen-2-yl)-1,2,5-thiadiazolidin-3-one 1,1-dioxide (19 g, 80% purity, 32.7 mmol) in dioxane (200 mL) was added bis(pinacolato)diboron (16.6 g, 65.3 mmol), potassium acetate (9.62 g, 98 mmol), and [1,1′-bis(diphenylphosphino)ferrocene]dichloropalladium(II) (2.87 g, 3.92 mmol). The mixture was heated to 100° C. After 3 h, the solvent was evaporated, and the residue was taken up in water (200 mL) and extracted with ethyl acetate (3×300 mL). The combined organic phases were washed with brine (2×100 mL), dried over Na₂SO₄, filtered, and concentrated. The residue was purified by silica gel chromatography (50% to 60% heptanes:ethyl acetate) to provide 5-(3-(benzyloxy)-1-fluoro-7-(4,4,5,5-tetramethyl-1,3,2-dioxaborolan-2-yl)naphthalen-2-yl)-1,2,5-thiadiazolidin-3-one 1,1-dioxide (16 g, 28.1 mmol, 86% yield, 90% purity) as a yellow solid. LCMS (NH₄HCO₃, ESI−): m/z 511.1 [M−H]^{- 1}H NMR (600 MHZ, DMSO-d₆) δ 8.30 (q, J=0.9 Hz, 1H), 7.88 (dd, J=8.5, 1.3 Hz, 1H), 7.80 (dd, J=8.3, 1.2 Hz, 1H), 7.56-7.51 (m, 2H), 7.48 (s, 1H), 7.42-7.36 (m, 2H), 7.36-7.31 (m, 1H), 5.30 (s, 2H), 4.49 (s, 2H), 1.34 (s, 12H).

### Step 2: 5-(3-(Benzyloxy)-1-fluoro-7-hydroxynaphthalen-2-yl)-1,2,5-thiadiazolidin-3-one 1,1-dioxide (3)

To a solution of 5-(3-(benzyloxy)-1-fluoro-7-(4,4,5,5-tetramethyl-1,3,2-dioxaborolan-2-yl)naphthalen-2-yl)-1,2,5-thiadiazolidin-3-one 1,1-dioxide (16 g, 78% purity, 28.1 mmol) in acetone (160 mL) at (° C.) was added a solution of potassium peroxymonosulfate (24.19 g, 39.3 mmol) in water (160 mL). After 1 h, the mixture was warmed to RT and stirred an additional 3 h. The acetone was removed under vacuum, and the remaining aqueous mixture treated with sodium thiosulfate pentahydrate (8.88 g, 56.2 mmol), stirred 15 min, and extracted with ethyl acetate (3×200 mL). The combined organic phases were washed with brine (2×100 mL), dried over Na₂SO₄, filtered, and concentrated to give 5-(3-(benzyloxy)-1-fluoro-7-hydroxynaphthalen-2-yl)-1,2,5-thiadiazolidin-3-one 1,1-dioxide (14 g, 24.4 mmol, 87% yield, 70% purity) as a dark solid. LCMS (NH₄HCO₃, ESI−): m/z 401.1 [M−H]^{- 1}H NMR (400 MHZ, DMSO-d₆) δ 4.51 (s, 2H), 5.22 (s, 2H), 7.16-7.19 (m, 2H), 7.33 (br d, J=7.0 Hz, 1H), 7.37 (br d, J=8.1 Hz, 3H), 7.51 (br d, J=7.1 Hz, 3H), 7.75 (br d, J=8.9 Hz, 1H).

### Step 3: tert-Butyl (2-((6-(benzyloxy)-7-(1,1-dioxido-4-oxo-1,2,5-thiadiazolidin-2-yl)-8-fluoronaphthalen-2-yl)oxy)ethyl)carbamate (4)

To a solution of 5-(3-(benzyloxy)-1-fluoro-7-hydroxynaphthalen-2-yl)-1,2,5-thiadiazolidin-3-one 1,1-dioxide (20 g, 70% purity, 34.8 mmol) and Cs₂CO₃ (45.3 g, 139 mmol) in DMF (200 mL) was added 2-((tert-butoxy carbonyl)amino)ethyl methanesulfonate (71.4 g, 209 mmol) in one portion, and the slurry was heated to 60° C. for 4 h. The reaction was cooled to RT and diluted with water (500 mL). The aqueous mixture was extracted with ethyl acetate (3×300 mL), and the combined organic layers were washed with brine (2×100 mL), dried over Na₂SO₄, filtered, and concentrated to obtain tert-butyl (2-((6-(benzyloxy)-7-(1,1-dioxido-4-oxo-1,2,5-thiadiazolidin-2-yl)-8-fluoronaphthalen-2-yl)oxy)ethyl)carbamate (19 g, 34.8 mmol, 79% yield) as yellow solid. LCMS (NH₄HCO₃, ESI−): m/z 544.0 [M−H]^{- 1}H NMR (400 MHZ, CDCl₃) δ 1.44 (s, 9H), 3.50 (br s, 2H), 3.94 (br t, J=4.8 Hz, 2H), 4.44 (br s, 2H), 5.09-5.14 (m, 2H), 6.93 (s, 1H), 7.06-7.12 (m, 2H), 7.15-7.22 (m, 1H), 7.25 (s, 1H), 7.29 (br s, 1H), 7.37 (br d, J=7.3 Hz, 2H), 7.51 (br d, J=8.7 Hz, 1H), 8.03 (s, 1H).

### Step 4: tert-Butyl (2-((7-(1,1-dioxido-4-oxo-1,2,5-thiadiazolidin-2-yl)-8-fluoro-6-hydroxynaphthalen-2-yl)oxy)ethyl)carbamate (5)

To a solution of tert-butyl (2-((6-(benzyloxy)-7-(1,1-dioxido-4-oxo-1,2,5-thiadiazolidin-2-yl)-8-fluoronaphthalen-2-yl)oxy)ethyl)carbamate (4 g, 7.3 mmol) in methanol (50 mL) at RT was added Pd(OH)₂ (1.0 g) under a N₂ atmosphere. The suspension was degassed (vacuum/purge H₂×3), and the mixture was stirred for 12 h under a hydrogen balloon atmosphere. After completion, the slurry was filtered through a celite pad and the filter cake was washed with methanol (100 mL). The filtrate was concentrated under reduced pressure and the crude product was purified by reversed phase column chromatography (50% water:acetonitrile) to give tert-butyl (2-((7-(1,1-dioxido-4-oxo-1,2,5-thiadiazolidin-2-yl)-8-fluoro-6-hydroxynaphthalen-2-yl)oxy)ethyl)carbamate (10 g, 21.96 mmol, 29% yield) as white solid. LCMS (NH₄HCO₃, ESI−): m/z 454.2 [M−H]^{- 1}H NMR (400 MHZ, DMSO-d₆) δ 1.38 (s, 9H), 3.34 (br d, J=5.6 Hz, 2H), 4.06 (br d, J=4.5 Hz, 2H), 4.44 (s, 2H), 7.03-7.07 (m, 2H), 7.14-7.22 (m, 2H), 7.71 (d, J=9.0 Hz, 1H), 10.12-10.35 (m, 1H).

### Step 5: 5-(7-(2-Aminoethoxy)-1-fluoro-3-hydroxynaphthalen-2-yl)-1,2,5-thiadiazolidin-3-one 1,1-dioxide, hydrochloric acid salt (6)

A solution of tert-butyl (2-((7-(1,1-dioxido-4-oxo-1,2,5-thiadiazolidin-2-yl)-8-fluoro-6-hydroxynaphthalen-2-yl)oxy)ethyl)carbamate (2 g, 90% purity, 3.95 mmol) in 4 M HCl in EtOAc (10 mL, 40.0 mmol) at RT was stirred for 1 h. The mixture was concentrated under reduced pressure. The residue was triturated with 95% i-PrOH, and the solid was collected by filtration and dried under vacuum to give 5-(7-(2-aminoethoxy)-1-fluoro-3-hydroxynaphthalen-2-yl)-1,2,5-thiadiazolidin-3-one 1,1-dioxide, hydrochloric acid salt (1.01 g, 2.52 mmol, 64% yield, 98% purity) as white solid.

LCMS (NH₄HCO₃, ESI−): m/z 354.0 [M−H]⁻

¹H NMR (400 MHZ, DMSO-d₆) δ 3.27 (br d, J=5.1 Hz, 2H), 4.18 (s, 2H), 4.27 (br t, J=5.0 Hz, 2H), 7.06 (s, 1H), 7.20 (dd, J=8.9, 2.3 Hz, 1H), 7.25 (d, J=2.0 Hz, 1H), 7.73 (d, J=9.0 Hz, 1H), 8.00 (br s, 3H), 9.77 (br s, 1H).

### 5-(7-(2-aminoethoxy)-3-(benzyloxy)-1-fluoronaphthalen-2-yl)-1,2,5-thiadiazolidin-3-one 1,1-dioxide (2)

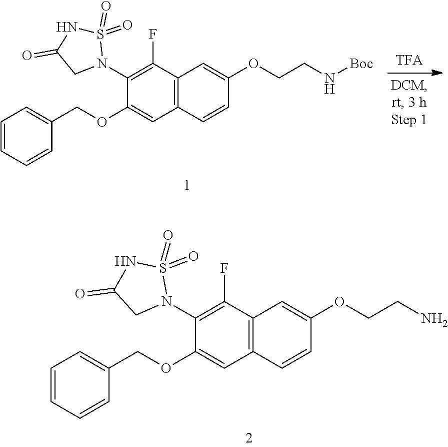 [SMILES: CC(C)(C)OC(=O)NCCOc1ccc2cc(OCc3ccccc3)c(N3CC(=O)NS3(=O)=O)c(F)c2c1 ; NCCOc1ccc2cc(OCc3ccccc3)c(N3CC(=O)NS3(=O)=O)c(F)c2c1]

### Step 1: 5-(7-(2-aminoethoxy)-3-(benzyloxy)-1-fluoronaphthalen-2-yl)-1,2,5-thiadiazolidin-3-one 1,1-dioxide (2)

Into a 50 mL round bottom flask containing a solution of tert-butyl N-[2-[[6-benzyloxy-8-fluoro-7-(1,1,4-trioxo-1,2,5-thiadiazolidin-2-yl)-2-naphthyl]oxy]ethyl]carbamate (1, 500 mg, 916.46 μmol) in DCM (5 mL) at 0° C. was added TFA (104.50 mg, 916.46 μmol, 70.61 μL) dropwise. The reaction mixture was stirred at RT for 3 h. The solvent was removed under reduced pressure, and the residue was triturated with diethyl ether (2×8 mL) to obtain 5-[7-(2-aminoethoxy)-3-benzyloxy-1-fluoro-2-naphthyl]-1,1-dioxo-1,2,5-thiadiazolidin-3-one (2, 520 mg, 873.81 μmol, 95.35% yield, TFA salt) as an off white solid.

LCMS (ES+): m/z 446.1 [M+H]⁺

### 3-(4-(((2-(2,6-dioxopiperidin-3-yl)-1,3-dioxoisoindolin-5-yl)amino)methyl)-1H-1,2,3-triazol-1-yl)propanoic acid (4)

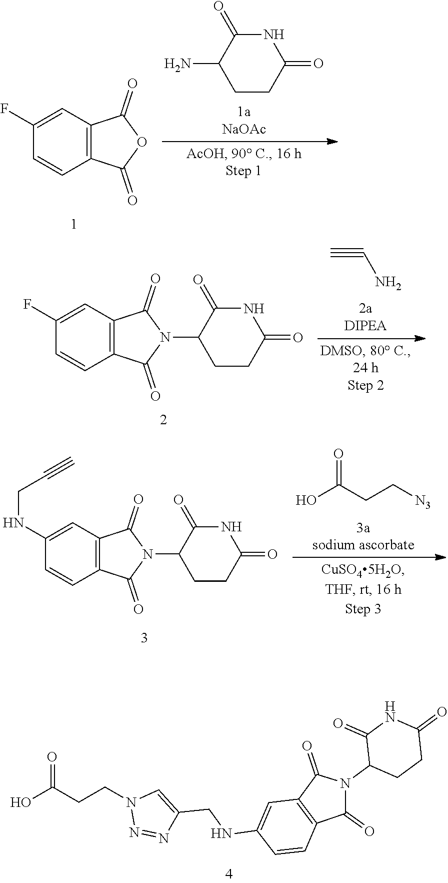

### Step 1: 2-(2,6-dioxo-3-piperidyl)-5-fluoro-isoindoline-1,3-dione (2)

Into a 250 mL three neck round bottom flask containing a well-stirred solution of 5-fluoroisobenzofuran-1,3-dione (1, 5 g, 30.10 mmol, 3.33 mL) and 3-aminopiperidine-2,6-dione (1a, 4.24 g, 33.11 mmol) in anhydrous acetic acid (50 mL) was added NaOAc (4.94 g, 60.20 mmol, 3.23 mL). The reaction was stirred at 80° C. for 16 h. The reaction mixture was concentrated to dryness, and the residue was diluted with ice-cold water (100 mL) to get a solid that was filtered, washed with pet-ether and dried to afford 2-(2,6-dioxo-3-piperidyl)-5-fluoro-isoindoline-1,3-dione (2, 8 g, 28.90 mmol, 96% yield) as a pale-brown solid.

LCMS (ES+): m/z 277 [M+H]⁺

### Step 2: 2-(2,6-dioxopiperidin-3-yl)-5-(prop-2-yn-1-ylamino)isoindoline-1,3-dione (3)

Into a 25 mL pressure tube containing a well-stirred suspension of 2-(2,6-dioxo-3-piperidyl)-5-fluoro-isoindoline-1,3-dione (2, 500 mg, 1.81 mmol) and prop-2-yn-1-amine (2a, 179.46 mg, 3.26 mmol, 208.68 μL) in anhydrous DMSO (5 mL) was added DIPEA (701.85 mg, 5.43 mmol, 945.89 μL). The tube was sealed, and the reaction mixture was stirred at 80° C. for 24 h. The reaction mixture was concentrated to dryness under reduced pressure. The resulting residue was diluted with ice-cold water (50 mL), and the precipitate thus formed was collected by filtration, washed with pet-ether, dried, and purified by flash column chromatography (neutral alumina, 10% MeOH/DCM) to afford 2-(2,6-dioxopiperidin-3-yl)-5-(prop-2-yn-1-ylamino)isoindoline-1,3-dione (3, 180 mg, 511.16 μmol, 28% yield) as a yellow solid.

LCMS (ES+): m/z 312.3 [M+H]⁺

### Step 3: 3-(4-(((2-(2,6-dioxopiperidin-3-yl)-1,3-dioxoisoindolin-5-yl)amino)methyl)-1H-1,2,3-triazol-1-yl)propanoic acid (4)

Into a 50 mL single neck round bottom flask containing a well-stirred solution of 2-(2,6-dioxo-3-piperidyl)-5-(prop-2-ynylamino)isoindoline-1,3-dione (3, 180 mg, 578.24 μmol) and 3-azidopropanoic acid (3a, 99.82 mg, 867.35 μmol) in anhydrous THF (5 mL) was added sodium ascorbate (229.10 mg, 1.16 mmol) and copper(II) sulphate pentahydrate (288.75 mg, 1.16 mmol). The reaction was stirred at ambient temperature for 16 h. The reaction mixture was poured into ice-cold water (10 mL), and the aqueous layer was extracted with ethyl acetate (2×15 mL). The organic layers were combined, washed with brine (10 mL), dried over anhydrous Na₂SO₄, filtered, and concentrated under reduced pressure. The residue was purified by reverse-phase preparative HPLC [Column: X BRIDGE C18 column (19×150) mm 5 micron; Mobile phase A: 0.1% TFA in water and Mobile phase B: MeCN] to afford 3-(4-(((2-(2,6-dioxopiperidin-3-yl)-1,3-dioxoisoindolin-5-yl)amino)methyl)-1H-1,2,3-triazol-1-yl)propanoic acid (4, 100 mg, 177.64 μmol, 31% yield, TFA salt). LCMS (ES+): m/z 427.0 [M+H]⁺

### 3-[4-[[[2-(2,6-dioxo-3-piperidyl)-1-oxo-isoindolin-5-yl]amino]methyl]pyrazol-1-yl]propanoic acid (6)

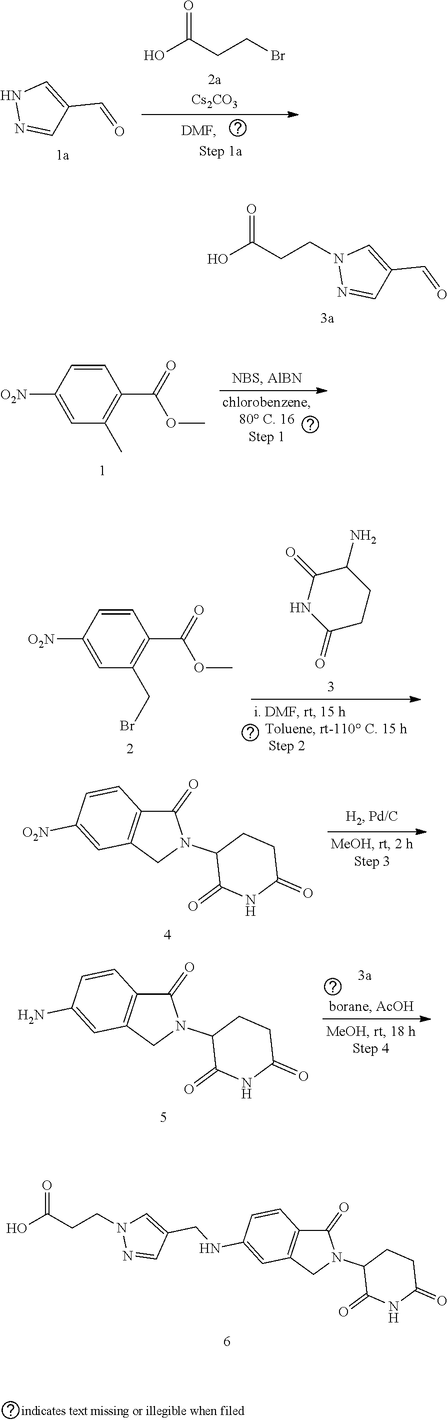

### Step 1a: 3-(4-formylpyrazol-1-yl)propanoic acid (3a)

Into a 50 mL single neck round bottom flask containing a well-stirred solution of 1H-pyrazole-4-carbaldehyde (1a, 1 g, 10.41 mmol) in DMF (10 mL) were added cesium carbonate (6.78 g, 20.81 mmol) followed by 3-bromopropanoic acid (2a, 1.91 g, 12.49 mmol, 1.29 mL). The reaction mixture was stirred at 100° C. for 6 h. The reaction mixture was concentrated under reduced pressure and diluted with water (45 mL). The reaction mixture was acidified (pH ˜6) using 1.5 N HCl and extracted with 10% MeOH in DCM (5×25 mL). The combined organic layer was washed with water (2×25 mL), brine (25 mL), dried over anhydrous sodium sulfate, and filtered. The solvent was removed under reduced pressure and the residue purified by flash silica gel (230-400) column chromatography (10-12% MeOH in DCM) to afford 3-(4-formylpyrazol-1-yl)propanoic acid (3a, 800 mg, 3.76 mmol, 36% yield) as a brown liquid. LCMS (ES+): m/z 169.0 [M+H]⁺

### Step 1: methyl 2-(bromomethyl)-4-nitro-benzoate (2)

Into a 250 mL single neck round bottom flask containing a well-stirred solution of methyl 4-bromo-2-methyl-benzoate (1, 10 g, 51.24 mmol) in chlorobenzene (100 mL) was added NBS (9.12 g, 51.24 mmol) followed by AIBN (841 mg, 5.124 mmol). The reaction mixture was heated at 80° C. for 16 h. The reaction mixture was cooled to RT, quenched with water (100 mL) and extracted with DCM (3×100 mL). The organic layer was washed with water (2×100 mL), brine (100 mL), dried over anhydrous sodium sulfate, filtered, and concentrated. The residue was purified by flash silica gel column chromatography (3-4% EtOAc in petroleum ether) to yield methyl 2-(bromomethyl)-4-nitro-benzoate (2, 10 g, 28.28 mmol, 55% yield) as a light-yellow liquid. GCMS: m/z 272.9

### Step 2: 3-(5-nitro-1-oxo-isoindolin-2-yl)piperidine-2,6-dione (4)

Into a 250 mL single neck round bottom flask containing a well-stirred solution of methyl 2-(bromomethyl)-4-nitro-benzoate (2, 10 g, 36.49 mmol, 77.5% purity) in DMF (100 mL) was added TEA (18.46 g, 182.44 mmol, 25.43 mL) followed by 3-aminopiperidine-2,6-dione; hydrochloride (3, 5.61 g, 34.08 mmol). The reaction mixture was stirred at RT for 15 h. The reaction was concentrated under reduced pressure, water (100 mL) was added and extracted with EtOAc (3×250 mL). The combined organic layer was washed with water (2×250 mL), brine (250 mL) and dried over anhydrous sodium sulfate and filtered. The organic layer was concentrated under reduced pressure. The residue was suspended in toluene (100 mL) and heated at 110° C. for 15 h. The reaction mixture was cooled to RT and concentrated under reduced pressure to afford 3-(5-nitro-1-oxo-isoindolin-2-yl)piperidine-2,6-dione (4, 8 g, 15.49 mmol, 42% yield) as a brown solid. LCMS (ES+): m/z 290.0 [M+H]⁺

### Step 3: 3-(5-amino-1-oxo-isoindolin-2-yl)piperidine-2,6-dione (5)

Into a 100 mL single neck round bottom flask containing a well-stirred solution of 3-(5-nitro-1-oxo-isoindolin-2-yl)piperidine-2,6-dione (4, 4 g, 13.83 mmol, 56% purity) in MeOH (50 mL) was added Pd/C (dry 10 wt. %) (1.47 g, 13.83 mmol) and stirred under hydrogen atmosphere at RT for 2 h. The reaction mixture was filtered through a pad of Celite and washed with MeOH (1 mL). The solvent was removed under reduced pressure to yield 3-(5-amino-1-oxo-isoindolin-2-yl)piperidine-2,6-dione (5, 2.5 g, 6.56 mmol, 85% yield) as a brown solid. LCMS (ES+): m/z 260.1 [M+H]⁺

### Step 4: 3-[4-[[[2-(2,6-dioxo-3-piperidyl)-1-oxo-isoindolin-5-yl]amino]methyl]pyrazol-1-yl]propanoic acid (6)

Into a 50 mL single neck round bottom flask containing a well-stirred solution of 3-(4-formylpyrazol-1-yl)propanoic acid (3a, 129.72 mg, 771.43 μmol, 79% purity) and 3-(5-amino-1-oxo-isoindolin-2-yl)piperidine-2,6-dione (5, 200 mg, 771.43 μmol) in MeOH (10 mL) was added AcOH (463.24 mg, 772.00 μmol) followed by portion-wise addition of 2-picoline borane complex (107.27 mg, 100.0 μmol) at 10° C. After stirring at RT for 18 h, water (1 mL) was added to the reaction mixture and concentrated to dryness under reduced pressure. The residue purified by reverse phase prep HPLC [Purification method: Column: X Bridge C18, 10 mm×250 mm, 5μ, Mobile phase A: 0.1% TFA in water and Mobile phase B: MeCN] to afford 3-[4-[[[2-(2,6-dioxo-3-piperidyl)-1-oxo-isoindolin-5-yl]amino]methyl] pyrazol-1-yl]propanoic acid (6, 130 mg, 230.47 μmol, 38% yield, TFA salt) as an-off white solid. LCMS (ES+): m/z 412.2 [M+H]⁺

### 4-(((1-(azetidin-3-yl)-1H-1,2,3-triazol-4-yl)methyl)amino)-2-(2,6-dioxopiperidin-3-yl)isoindoline-1,3-dione (8)

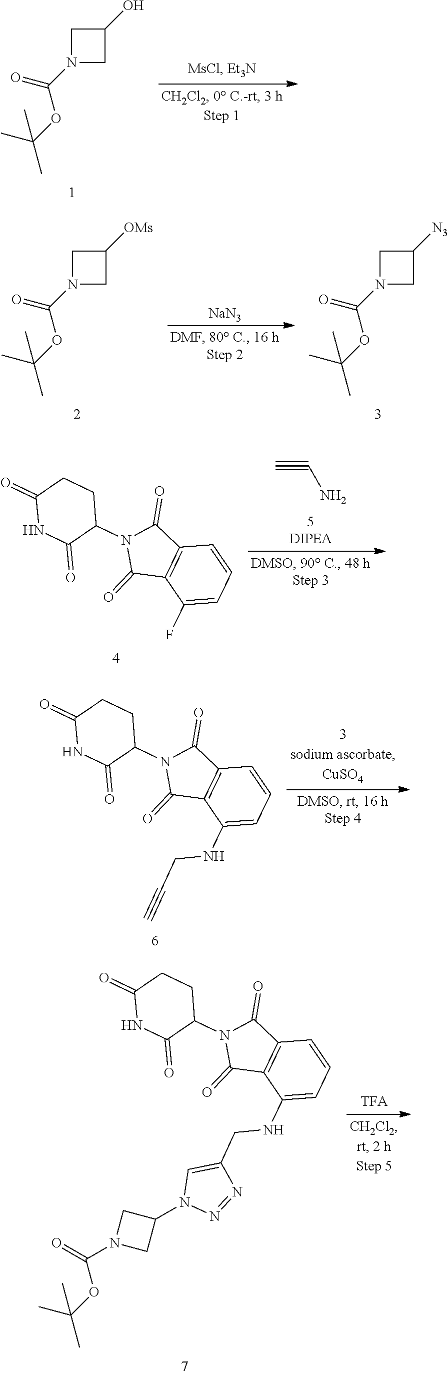

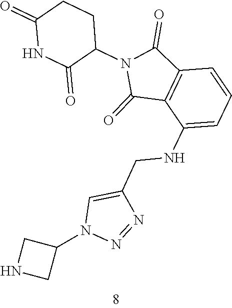 [SMILES: O=C1CCC(N2C(=O)c3cccc(NCc4cn(C5CNC5)nn4)c3C2=O)C(=O)N1]

### Step 1: tert-butyl 3-methylsulfonyloxyazetidine-1-carboxylate (2)

Into a 100 mL two neck round bottom flask containing a well-stirred solution of tert-butyl 3-hydroxyazetidine-1-carboxylate (1, 2.5 g, 14.43 mmol) in DCM (25 mL) were added Et₃N (3.65 g, 36.08 mmol, 5.03 mL) and methanesulfonyl chloride (1.65 g, 14.43 mmol, 1.12 mL) at 0° C. After stirring at RT for 3 h, the reaction was quenched with water (100 mL) and extracted with DCM (3×150 mL). The organic layers were combined, washed with water (150 mL), brine (100 mL), dried over anhydrous sodium sulphate, filtered and concentrated under reduced pressure to afford tert-butyl 3-methylsulfonyloxyazetidine-1-carboxylate (2, 3.5 g, 13.91 mmol, 96% yield) as an off-white solid, which was used in the next step without further purification. LCMS (ES+): m/z 152.0 [M-Boc+H]⁺

### Step 2: tert-butyl 3-azidoazetidine-1-carboxylate (3)

Into a 100 mL two neck round bottom flask containing a well-stirred solution of tert-butyl 3-methylsulfonyloxyazetidine-1-carboxylate (2, 3.5 g, 13.93 mmol) in DMF (30 mL) was added NaN₃ (2.26 g, 34.82 mmol) and the reaction mixture was stirred at 80° C. for 16 h. The reaction was quenched with ice water (200 mL) and extracted with EtOAc (2×200 mL). The organic layers were combined, washed with water (100 mL), brine (100 mL), dried over anhydrous sodium sulphate, filtered, and concentrated under reduced pressure to afford tert-butyl 3-azidoazetidine-1-carboxylate (3, 2.5 g, 12.54 mmol, 90% yield) as viscous liquid, which was used in the next step without further purification. ¹HNMR (400 MHZ, DMSO-d₆): δ 4.23-4.17 (m, 3H), 3.91-3.89 (m, 2H), 1.45 (s, 9H)

### Step 3: 2-(2,6-dioxo-3-piperidyl)-4-(prop-2-ynylamino)isoindoline-1,3-dione (6)

Into a 100 mL two neck round bottom flask containing a well stirred solution of 2-(2,6-dioxo-3-piperidyl)-4-fluoro-isoindoline-1,3-dione (4, 8.0 g, 28.96 mmol) and propargylamine (5, 2.39 g, 43.44 mmol, 2.78 mL) in DMSO (80 mL) was added DIPEA (22.46 g, 173.77 mmol, 30.27 mL), and the reaction mixture was stirred at 90° C. for 48 h. The reaction mixture was diluted with water (100 mL) and extracted with EtOAc (3×100 mL). The organic layers were combined, washed with ice water (100 mL), brine (100 mL), dried over sodium sulphate, filtered and concentrated under reduced pressure. The residue was purified by silica gel chromatography (50% EtOAc in petroleum ether) to obtain 2-(2,6-dioxo-3-piperidyl)-4-(prop-2-ynylamino)isoindoline-1,3-dione (6, 1.0 g, 2.95 mmol, 10% yield) as light yellow solid. LCMS (ES+): m/z 312.0 [M+H]⁺

### Step 4: tert-butyl 3-[4-[[[2-(2,6-dioxo-3-piperidyl)-1,3-dioxo-isoindolin-4-yl]amino]methyl]triazol-1-yl]azetidine-1-carboxylate (7)

Into a 50 mL single neck round bottom flask containing a well-stirred solution of 2-(2,6-dioxo-3-piperidyl)-4-(prop-2-ynylamino)isoindoline-1,3-dione (6, 100 mg, 321.24 μmol) in DMSO (2 mL) were added copper sulphate (5.13 mg, 32.12 μmol, 1.42 μL), tert-butyl 3-azidoazetidine-1-carboxylate (3, 63.68 mg, 321.24 μmol) and (+)-sodium-L-ascorbate (19.09 mg, 96.37 μmol) at RT. After 16 h, the reaction mixture was diluted with water (50 mL) and extracted with EtOAc (3×50 mL). The organic layers were combined, washed with brine, dried over sodium sulphate, filtered and concentrated under reduced pressure. The residue was triturated with Et₂O, filtered and dried to afford tert-butyl 3-[4-[[[2-(2,6-dioxo-3-piperidyl)-1,3-dioxo-isoindolin-4-yl]amino]methyl]triazol-1-yl]azetidine-1-carboxylate (7, 150 mg, 183.32 μmol, 57% yield) as a light yellow solid. LCMS (ES+): m/z 510.3 [M+H]⁺

### Step 5: 4-[[1-(azetidin-3-yl)triazol-4-yl]methylamino]-2-(2,6-dioxo-3-piperidyl) isoindoline-1,3-dione (8)

Into a 50 mL single neck round bottom flask containing a well-stirred solution of tert-butyl 3-[4-[[[2-(2,6-dioxo-3-piperidyl)-1,3-dioxo-isoindolin-4-yl]amino]methyl]triazol-1-yl]azetidine-1-carboxylate (7, 150 mg, 294.40 μmol) in DCM (5 mL) was added TFA (335.67 mg, 2.94 mmol, 226.81 μL) at RT. After 2 h, the reaction mixture was concentrated under reduced pressure to obtain 4-[[1-(azetidin-3-yl)triazol-4-yl]methylamino]-2-(2,6-dioxo-3-piperidyl) isoindoline-1,3-dione (8, 100 mg, 135.57 μmol, 46% yield, TFA salt) as brown sticky liquid, which was used in the next step without further purification. LCMS (ES+): m/z 410.2 [M+H]⁺

### 3-(4-(2-((2-(2,6-dioxopiperidin-3-yl)-1,3-dioxoisoindolin-4-yl)amino)propan-2-yl)-1H-1,2,3-triazol-1-yl)propanoic acid (5)

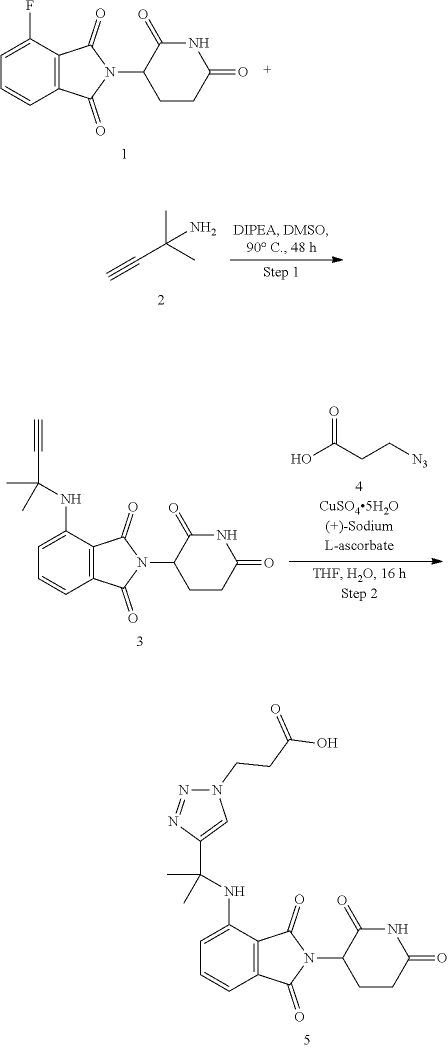

### Step 1: 4-(1,1-dimethylprop-2-ynylamino)-2-(2,6-dioxo-3-piperidyl)isoindoline-1,3-dione (3)

Into a pressure tube containing a well-stirred solution of 2-(2,6-dioxo-3-piperidyl)-4-fluoro-isoindoline-1,3-dione (1, 0.500 mg, 1.81 mmol) in DMSO (5 mL) was added 2-methylbut-3-yn-2-amine (2, 225.72 mg, 2.72 mmol, 285.72 μL) and DIPEA (1.40 g, 10.86 mmol, 1.89 mL). The vial was sealed and the reaction mixture was stirred at 90° C. for 48 h. The reaction mixture was poured into ice water and the precipitate was collected by filtration, washed with water and dried under vacuum to yield 4-(1,1-dimethylprop-2-ynylamino)-2-(2,6-dioxo-3-piperidyl)isoindoline-1,3-dione (3, 160 mg, 457.35 μmol, 26% yield) as a green solid, which was used in the next step without further purification. LCMS (ES+): m/z 340.3 [M+H]⁺

### Step 2: 3-(4-(2-((2-(2,6-dioxopiperidin-3-yl)-1,3-dioxoisoindolin-4-yl)amino)propan-2-yl)-1H-1,2,3-triazol-1-yl)propanoic acid (5)

Into a 50 mL single neck round bottom flask containing a well-stirred solution of 4-(1,1-dimethylprop-2-ynylamino)-2-(2,6-dioxo-3-piperidyl)isoindoline-1,3-dione (3, 0.160 g, 471.50 μmol) in THF (4 mL) and water (1 mL) were added 3-azidopropanoic acid (4, 54.26 mg, 471.50 μmol), copper(II) sulfate pentahydrate (117.73 mg, 471.50 μmol) and (+)-sodium L-ascorbate (93.41 mg, 471.50 μmol). The reaction mixture was stirred at ambient temperature for 16 h. After completion of the reaction, the reaction mixture was quenched with water (50 mL) and extracted with ethyl acetate (2×100 mL). The combined organic layer was washed with water (100 mL) and brine (50 mL), dried over anhydrous Na₂SO₄, filtered and the solvent removed under reduced pressure to yield 3-(4-(2-((2-(2,6-dioxopiperidin-3-yl)-1,3-dioxoisoindolin-4-yl)amino)propan-2-yl)-1H-1,2,3-triazol-1-yl)propanoic acid (5, 0.090 g, 180.22 μmol, 42% yield) as a green solid. LCMS (ES−): m/z 453.2 [M−H]⁻

### 3-[4-[[2-(2,6-dioxo-3-piperidyl)-1-oxo-isoindolin-4-yl]oxymethyl]triazol-1-yl]propanoic acid (6)

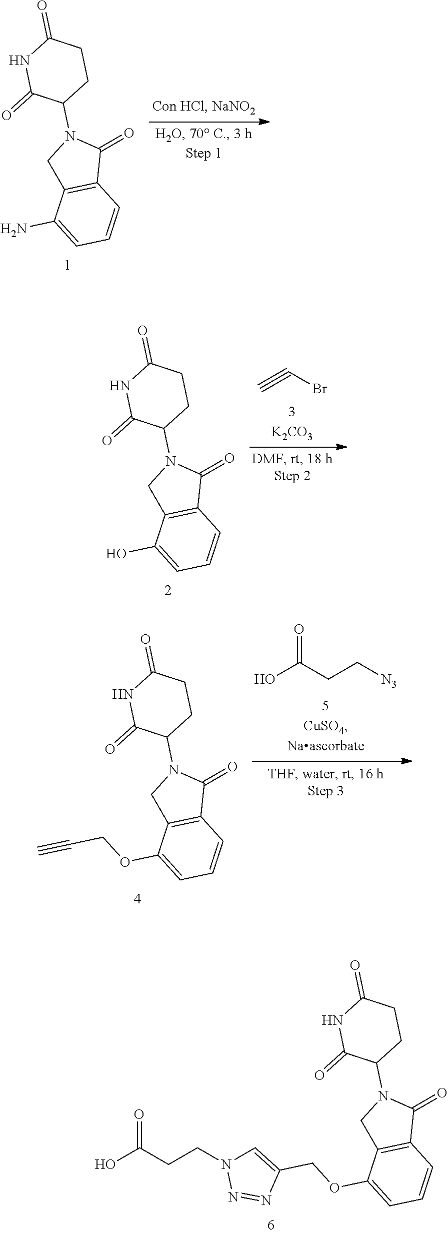

### Step 1: 3-(4-hydroxy-1-oxo-isoindolin-2-yl)piperidine-2,6-dione (2)

Into a 250 mL single neck round bottom flask containing a well-stirred solution of 3-(4-amino-1-oxo-isoindolin-2-yl)piperidine-2,6-dione (1, 4 g, 15.43 mmol) in water (50 mL) was added concentrated HCl (aq 30%, 10 mL) at 0° C. After 10 min, a solution of NaNO₂ (1.60 g, 23.14 mmol) in water (10 mL) was added dropwise over 5 min. The reaction mixture was allowed to come to RT and then heated at 70° C. for 3 h. The reaction mixture was filtered to obtain a brown solid, which was purified by reverse phase column chromatography (C18-column; Mobile phase A: 0.1% HCOOH in water and Mobile phase B: MeCN) to afford 3-(4-hydroxy-1-oxo-isoindolin-2-yl)piperidine-2,6-dione (2, 1.5 g, 5.59 mmol) as a light brown solid. LCMS (ES+): m/z 261.1 [M+H]⁺

### Step 2: 3-(1-oxo-4-prop-2-ynoxy-isoindolin-2-yl)piperidine-2,6-dione (4)

Into a 50 mL single neck round bottom flask containing a well-stirred solution of 3-(4-hydroxy-1-oxo-isoindolin-2-yl)piperidine-2,6-dione (2, 1 g, 3.84 mmol) in DMF (20 mL) was added K₂CO₃ (637.29 mg, 4.61 mmol). After 10 min, 3-bromoprop-1-yne, 80% in toluene (3, 685.66 mg, 4.61 mmol, 0.857 mL) was added dropwise and the reaction mixture was stirred at RT for 16 h. The reaction was quenched with water (80 mL) and extracted with EtOAc (3×40 mL). The combined organic layer was washed with cold water (2×40 mL), brine (50 mL), dried over anhydrous sodium sulfate, filtered and concentrated under reduced pressure. The residue was purified by reverse phase preparative HPLC [Purification method: Column: Sunfire C18 (150×19 mm), 5 μm; Mobile phase A: 0.1% TFA in water and Mobile phase B: MeCN] to afford 3-(1-oxo-4-prop-2-ynoxy-isoindolin-2-yl)piperidine-2,6-dione (4, 250 mg, 830.81 μmol, 22% yield) as an-off white solid. LCMS (ES+): m/z 299.0 [M+H]⁺

### Step 3: 3-[4-[[2-(2,6-dioxo-3-piperidyl)-1-oxo-isoindolin-4-yl]oxymethyl]triazol-1-yl]propanoic acid (6)

Into a 25 mL single neck round bottom flask containing a well-stirred solution of 3-(1-oxo-4-prop-2-ynoxy-isoindolin-2-yl)piperidine-2,6-dione (4, 240 mg, 804.58 μmol) and 3-azidopropanoic acid (5, 185.20 mg, 1.61 mmol) in a mixture of THF (6 mL), DMSO (1 mL) and water (2 mL) at RT was added (+)-sodium-L-ascorbate (159.40 mg, 804.58 μmol) followed by CuSO₄ (128.42 mg, 804.58 μmol). After 16 h, the reaction mixture was filtered through a pad of Celite, washing with DMSO (10 mL). The solvent was removed under reduced pressure and the residue purified by reverse phase preparative HPLC [Purification method: Column: Atlantis C18 (150×19 mm), 5 μm; Mobile phase A: 0.1% TFA in water and Mobile phase B: MeCN] to afford 3-[4-[[2-(2,6-dioxo-3-piperidyl)-1-oxo-isoindolin-4-yl]oxymethyl]triazol-1-yl]propanoic acid (6, 210 mg, 391.41 μmol, 49% yield, TFA salt) as a white solid. LCMS (ES+): m/z 414.1 [M+H]⁺

### 3-[4-[[2-(2,6-dioxo-3-piperidyl)-1-oxo-isoindolin-5-yl]oxymethyl]triazol-1-yl]propanoic acid (7)

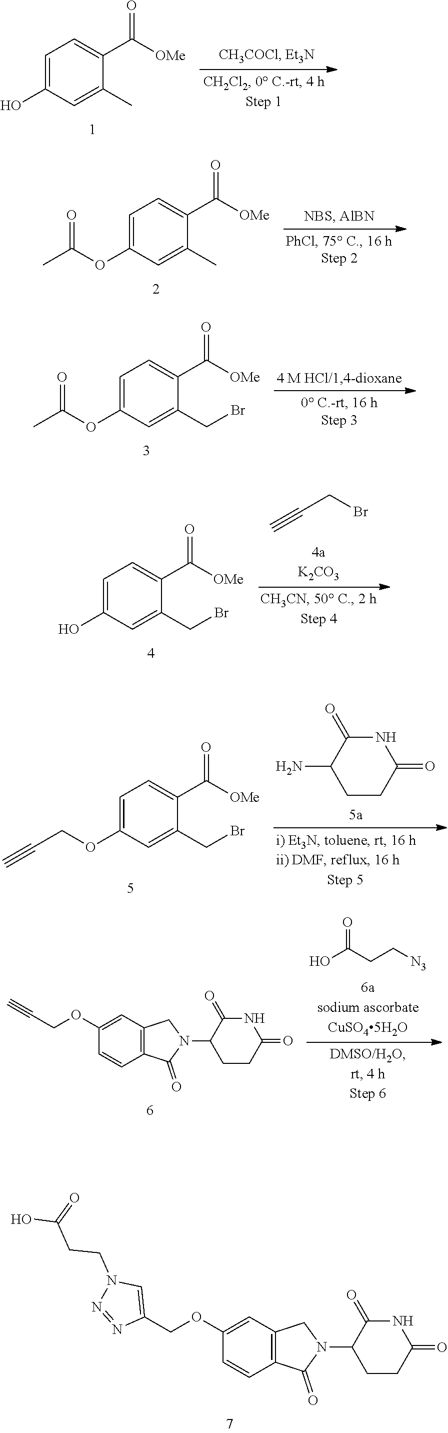

### Step 1: methyl 4-acetoxy-2-methylbenzoate (2)

Into a 500 mL single neck round bottom flask containing a well-stirred solution of methyl 4-hydroxy-2-methyl-benzoate (1, 8 g, 48.14 mmol) in CH₂Cl₂ (250 mL) were added Et₃N (12.18 g, 120.36 mmol, 16.78 mL) and acetyl chloride (5.67 g, 72.21 mmol, 4.39 mL). The resulting mixture was stirred at RT for 4 h. The reaction mixture was diluted with water (100 mL) and extracted with CH₂Cl₂ (2×200 mL). The organic layers were combined, dried over anhydrous Na₂SO₄, filtered and concentrated under reduced pressure. The residue was purified by silica gel chromatography (30% EtOAc in petroleum ether) to afford methyl 4-acetoxy-2-methyl-benzoate (2, 7.4 g, 35.54 mmol, 74% yield) as a pale yellow liquid. GCMS: m/z 208

### Step 2: methyl 4-acetoxy-2-(bromomethyl)benzoate (3)

Into a 250 mL single neck round bottom flask containing a well-stirred solution of methyl 4-acetoxy-2-methyl-benzoate (2, 7.4 g, 0.035 mol) in chlorobenzene (100 mL) were added NBS (7.59 g, 0.426 mol) and AIBN (0.58 g, 0.0035 mol). The resulting solution was stirred at 75° C. for 16 h. The reaction was quenched with water (100 mL) and extracted with dichloromethane (2×200 mL). The organic layers were combined, dried over anhydrous Na₂SO₄, filtered and concentrated under reduced pressure. The residue was purified by silica gel chromatography (20% EtOAc in petroleum ether) to afford methyl 4-acetoxy-2-(bromomethyl)benzoate (3, 5 g, 0.017 mol, 41% yield) as a viscous brown liquid. GCMS: m/z 285

### Step 3: methyl 2-(bromomethyl)-4-hydroxybenzoate (4)

Into a 500 mL single neck round bottom flask containing a well-stirred solution of methyl 4-acetoxy-2-(bromomethyl)benzoate (3, 5 g, 17.42 mmol) in 1,4-dioxane (50 mL) was added HCl (4.0 M in dioxane) (100 mL) at 0° C. The resulting mixture was stirred at RT for 16 h. The solvent was removed under reduced pressure and the residue purified by silica gel chromatography (20% EtOAc in petroleum ether) to obtain methyl 2-(bromomethyl)-4-hydroxy-benzoate (4, 2.7 g, 11.02 mmol, 63% yield) as an off-white solid. GCMS: m/z 243.9

### Step 4: methyl 2-(bromomethyl)-4-prop-2-ynoxy-benzoate (5)

Into a 100 mL single neck round bottom flask containing a well-stirred solution of anhydrous K₂CO₃ (1.56 g, 11.26 mmol) in CH₃CN (10 mL) were added 3-bromoprop-1-yne (4a, 5.82 g, 48.97 mmol) followed by methyl 2-(bromomethyl)-4-hydroxy-benzoate (4, 2.4 g, 9.79 mmol) in CH₃CN (10 mL). The reaction was heated at 50° C. for 2 h. The solvent was removed under reduced pressure, and the residue partitioned between EtOAc (50 mL) and water (50 mL). The organic layer was separated, washed with brine (25 mL), dried over anhydrous Na₂SO₄, filtered and concentrated under reduced pressure. The residue was purified by silica gel chromatography (0)-100% EtOAc in petroleum ether) to obtain methyl 2-(bromomethyl)-4-prop-2-ynoxy-benzoate (5, 0.75 g, 2.65 mmol, 27% yield) as a pale yellow semi-solid. ¹HNMR (400 MHZ, DMSO-d₆): δ 7.92 (d, J=8.80 Hz, 1H), 7.22 (d, J=2.40 Hz, 1H), 7.07 (dd, J=2.80, 8.80 Hz, 1H), 5.02 (s, 2H), 4.92 (s, 2H), 3.84 (s, 3H), 3.34 (s, 1H)

### Step 5: 3-(1-oxo-5-prop-2-ynoxy-isoindolin-2-yl)piperidine-2,6-dione (6)

Into a 50 mL single neck round bottom flask containing a well-stirred solution of methyl 2-(bromomethyl)-4-prop-2-ynoxy-benzoate (5, 0.75 g, 2.65 mmol) in anhydrous toluene (10 mL) were added 3-aminopiperidine-2,6-dione hydrochloride (5a, 654.02 mg, 3.97 mmol) and Et₃N (804.18 mg, 7.95 mmol, 1.11 mL). After 16 h, water (50 mL) was added to the reaction mixture and extracted with EtOAc (3×150 mL). The organic layers were combined, dried over anhydrous Na₂SO₄ and filtered. The filtrate was concentrated under reduced pressure to obtain a crude residue, which was taken up in anhydrous DMF (10 mL) and refluxed for 16 h. The reaction mixture was allowed to come to RT the solvent removed under reduced pressure. The residue was triturated with MTBE (3×25 mL) to afford 3-(1-oxo-5-prop-2-ynoxy-isoindolin-2-yl)piperidine-2,6-dione (6, 1.2 g, 935.68 μmol, 35% yield) as a grey solid. The material was used in the next step without further purification. LCMS (ES+): m/z 299.1 [M+H]⁺

### Step 6: 3-[4-[[2-(2,6-dioxo-3-piperidyl)-1-oxo-isoindolin-5-yl]oxymethyl]triazol-1-yl]propanoic acid (7)

Into a 50 mL single neck round bottom flask containing well-stirred solution of 3-azidopropanoic acid (6a, 350 mg, 3.04 mmol) and 3-(1-oxo-5-prop-2-ynoxy-isoindolin-2-yl)piperidine-2,6-dione (6, 907.13 mg, 3.04 mmol) in DMSO (5 mL) and water (5 mL) were added sodium ascorbate (602.46 mg, 3.04 mmol) and copper(II) sulphate pentahydrate (759.31 mg, 3.04 mmol). After 4 h, the reaction mixture was diluted with water (10 mL) and extracted with ethyl acetate (2×10 mL). The organic layers were combined, dried over anhydrous Na₂SO₄ and filtered. The filtrate was concentrated under reduced pressure, and the residue purified by reverse-phase preparatory HPLC [YMC C18 (150×20) mm, 5.0 μm with Solvent A: 10 mm Ammonium acetate in water; Solvent B: Acetonitrile] to obtain 3-[4-[[2-(2,6-dioxo-3-piperidyl)-1-oxo-isoindolin-5-yl]oxymethyl]triazol-1-yl]propanoic acid (7, 100 mg, 240.14 μmol, 8% yield) as a pale yellow solid. LCMS (ESI+): m/z 414.0 [M+H]⁺

### 3-(4-(((2-(2,6-dioxopiperidin-3-yl)-1-oxoisoindolin-4-yl)oxy)methyl)-1H-pyrazol-1-yl)propanoic acid (9)

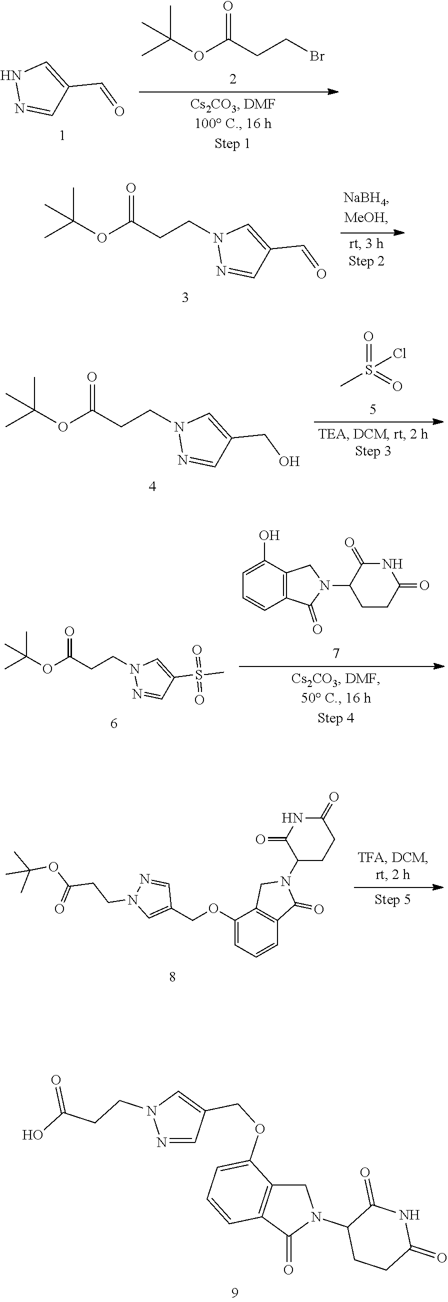

### Step 1: tert-butyl 3-(4-formyl-1H-pyrazol-1-yl)propanoate (3)

Into a 100 mL single neck round bottom flask containing well-stirred solution of 1H-pyrazole-4-carbaldehyde (1, 1 g, 10.41 mmol) in anhydrous DMF (10 mL) was added cesium carbonate (6.78 g, 20.81 mmol) at 0° C. under nitrogen atmosphere. After 15 min, tert-butyl 3-bromopropanoate (2, 4.35 g, 20.81 mmol, 2.15 mL) was added. The resulting suspension was stirred at 100° C. for 16 h. The reaction was quenched with water (30 mL) and extracted with EtOAc (3×20 mL). The combined organic layer was washed with brine (20 mL), dried over anhydrous Na₂SO₄, filtered and concentrated under reduced pressure. The residue was purified by silica gel chromatography (5% MeOH in DCM) to yield tert-butyl 3-(4-formylpyrazol-1-yl)propanoate (3, 2 g, 8.63 mmol, 83% yield) as colorless liquid.

¹H-NMR (400 MHZ, DMSO-d₆): δ 9.79 (s, 1H), 8.46 (s, 1H), 7.99 (s, 1H), 4.38 (t, J=8.00 Hz. 2H), 2.82 (t, J=8.00 Hz, 2H), 1.35 (s, 9H).

### Step 2: tert-butyl 3-(4-(hydroxymethyl)-1H-pyrazol-1-yl)propanoate (4)

Into a 50 mL single neck round bottom flask containing well-stirred solution of tert-butyl 3-(4-formylpyrazol-1-yl)propanoate (3, 2 g, 8.92 mmol) in anhydrous methanol (15 mL) was added sodium borohydride (506.11 mg, 13.38 mmol, 473.00 μL) at 0° C. under nitrogen atmosphere. The reaction mixture was stirred at RT for 3 h. The solvent was removed under reduced pressure and quenched with water (30 mL). The aqueous layer was extracted with EtOAc (2×20 mL). The organic layer was dried with anhydrous Na₂SO₄, filtered and concentrated under reduced pressure. The residue was purified by silica gel chromatography (80% EtOAc in petroleum ether) to obtain tert-butyl 3-[4-(hydroxymethyl)pyrazol-1-yl]propanoate (4, 1.2 g, 4.34 mmol, 49% yield) as colorless liquid. LCMS (ES+): m/z 227.2 [M+H]⁺

### Step 3: tert-butyl 3-(4-(((methylsulfonyl)oxy)methyl)-1H-pyrazol-1-yl)propanoate (6)

Into a 25 mL single neck round bottom flask containing well-stirred solution of tert-butyl 3-[4-(hydroxymethyl)pyrazol-1-yl]propanoate (4, 1.2 g, 5.30 mmol) in anhydrous DCM (10 mL) were added TEA (1.61 g, 15.91 mmol, 2.22 mL) and methanesulfonyl chloride (5, 911.26 mg, 7.96 mmol, 615.72 μL) at (° C.) The mixture was stirred at RT for 2 h. The reaction was quenched with water (30 mL) and extracted with DCM (2×20 mL). The combined organic layer was dried over anhydrous Na₂SO₄, filtered and concentrated under reduced pressure to obtain tert-butyl 3-[4-(methylsulfonyloxymethyl)pyrazol-1-yl] propanoate (6, 750) mg, 2.46 mmol, 47% yield) as a colorless liquid, which was used without further purification.

### Step 4: tert-butyl 3-(4-(((2-(2,6-dioxopiperidin-3-yl)-1-oxoisoindolin-4-yl)oxy)methyl)-1H-pyrazol-1-yl)propanoate (8)

Into a 50 mL pressure tube containing a well-stirred solution of 3-(4-hydroxy-1-oxo-isoindolin-2-yl)piperidine-2,6-dione (7, 500 mg, 1.92 mmol) in anhydrous DMF (10 mL) were added tert-butyl 3-[4-(methylsulfonyl oxymethyl)pyrazol-1-yl]propanoate (6, 877.14 mg, 2.88 mmol) and cesium carbonate (1.25 g, 3.84 mmol). The tube was sealed, and the mixture was stirred at 50° C. for 16 h. The reaction was quenched with water (30 mL) and extracted with EtOAc (2×20 mL). The combined organic layer was dried over anhydrous Na₂SO₄, filtered, and concentrated under reduced pressure. The crude residue contains a mixture of two monoalkylated products (16% and 17%) and dialkylated product (10%). The mixture was first purified by silica gel chromatography (5% MeOH in DCM) to give a mixture of the two monoalkylated products. This mixture of the two monoalkylated products was then purified by reverse phase prep HPLC [Purification method: Column: Sunfire C18 (19×150 mm) 5 micron, mobile phase: 0.1% FA in water and MeCN] to obtain tert-butyl 3-[4-[[2-(2,6-dioxo-3-piperidyl)-1-oxo-isoindolin-4-yloxymethyl]pyrazol-1-yl]propanoate (8, 70 mg, 107.58 μmol, 6% yield) as an off-white solid. LCMS (ES+): m/z 469.1 [M+H]⁺

### Step 5: 3-(4-(((2-(2,6-dioxopiperidin-3-yl)-1-oxoisoindolin-4-yl)oxy)methyl)-1H-pyrazol-1-yl)propanoic acid (9)

Into a 10 mL single neck round bottom flask containing a well-stirred solution of tert-butyl 3-[4-[2-(2,6-dioxo-3-piperidyl)-1-oxo-isoindolin-4-yl]oxymethyl]pyrazol-1-yl]propanoate (8, 70 mg, 107.58 μmol) in anhydrous DCM (2 mL) was added TFA (0.5 mL, 6.49 mmol) at 0° C. under nitrogen atmosphere. The mixture was stirred at RT for 2 h. The solvent was removed under reduced pressure and azeotroped with toluene to afford 3-[4-[[2-(2,6-dioxo-3-piperidyl)-1-oxo-isoindolin-4-yl]oxymethyl]pyrazol-1-yl]propanoic acid (9, 65 mg, 101.19 μmol, 92% yield) as an off-white solid. LCMS (ES+): m/z 413.2 [M+H]⁺

### 2-(4-(1-(2,6-dioxopiperidin-3-yl)-3-methyl-2-oxo-2,3-dihydro-1H-benzo[d]imidazol-5-yl)piperidin-1-yl)acetic acid (13)

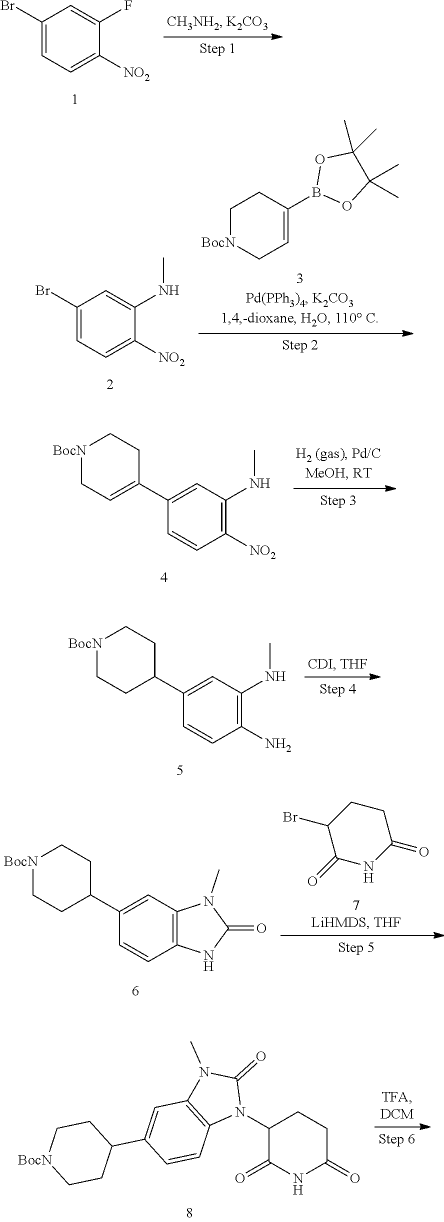

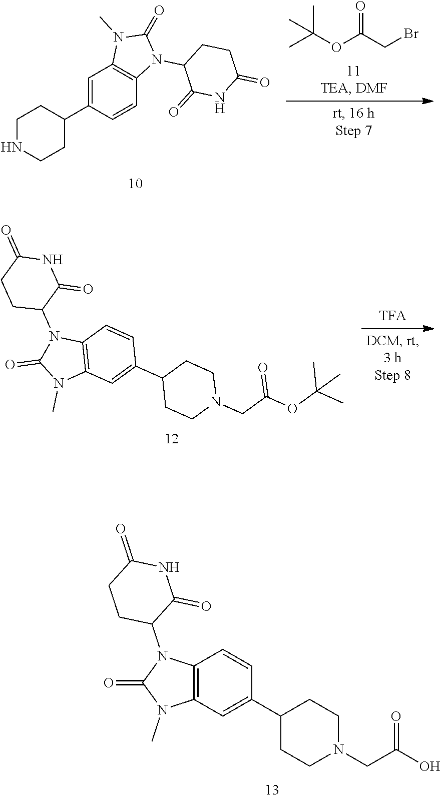

### Step 1: 5-bromo-N-methyl-2-nitroaniline (2)

To a stirred solution of 4-bromo-2-fluoro-1-nitrobenzene (1, 300 g, 1.36 mol) in DCM (3 L) were added K₂CO₃ (0.94 Kg, 6.8 mol) and methylamine (2M in THF) (2.04 L, 4.09 mol) at RT and stirred for 16 h. Two batches of the reaction were combined. After completion of reaction, the reaction mixture was diluted with water (3.0 L) and extracted with DCM (2.5 L×2). The combined organic layer was washed with saturated sodium bicarbonate solution (1.5 L×2) and brine (1.5 L×2). The organic layer was dried over sodium sulphate, filtered and solvent removed under reduced pressure to obtain 5-bromo-N-methyl-2-nitroaniline (2, 600 g, 95% yield) as a yellow solid. LCMS (ES+): m/z 231.1 [M+H]⁺

### Step 2: tert-butyl 4-(3-(methylamino)-4-nitrophenyl)-3,6-dihydropyridine-1(2H)-carboxylate (4)

To a stirred solution of 5-bromo-N-methyl-2-nitroaniline (2, 75.0 g, 0.326 mol) in 1,4-dioxane (1.2 L) and water (0.3 L) was added K₂CO₃ (270.3 g, 1.956 mol) and the mixture was stirred for 5 min. tert-Butyl 4-(4,4,5,5-tetramethyl-1,3,2-dioxaborolan-2-yl)-3,6-dihydropyridine-1 (2H)-carboxylate (3, 151.0 g, 0.489 mol) was added to the reaction mixture under nitrogen atmosphere and the reaction mixture was purged with nitrogen for 10 min. Palladium(0) tetrakis(triphenylphosphine) (37.66 g, 0.032 mol) was added to the reaction under nitrogen atmosphere. After purging with nitrogen for 10 min, the reaction was stirred at 110° C. for 4 h. Two batches of the reaction were combined. The reaction mixture was cooled to RT and filtered through celite. The filtrate was diluted with water (1.5 L) and extracted with ethyl acetate (500 mL×2). The combined organic layer was washed with brine, dried over sodium sulphate, filtered and solvent removed under reduced pressure. The residue was purified by silica gel chromatography (0)-20% EtOAc in petroleum ether as an eluent) to obtain tert-butyl 4-(3-(methylamino)-4-nitrophenyl)-3,6-dihydropyridine-1(2H)-carboxylate (4, 150 g, 69% yield) as a red solid. LCMS (ES+): m/z 334.3 [M+H]⁺

### Step 3: tert-butyl 4-(4-amino-3-(methylamino)phenyl)piperidine-1-carboxylate (5)

A solution of tert-butyl 4-(3-(methylamino)-4-nitrophenyl)-3,6-dihydropyridine-1(2H)-carboxylate (4, 50 g, 0.149 mol) in methanol (IL) in a Parr-shaker flask was degassed. Palladium on carbon (10%, wet) (25.0 g) was added and the reaction mixture was put under an atmosphere of hydrogen (70-75 psi). The reaction progress was monitored by TLC/LCMS. Four batches were combined. After 8 h, the reaction mixture was filtered through Celite, washing with methanol. The filtrate was evaporated under reduced pressure and the residue purified by silica gel chromatography (0)-20% ethyl acetate and petroleum ether as an eluent) to obtain tert-butyl 4-(4-amino-3-(methylamino)phenyl)piperidine-1-carboxylate (5, 120.0 g, 65% yield) as dark brown solid. LCMS (ES−): m/z 304.2 [M−H]⁻

### Step 4: tert-butyl 4-(3-methyl-2-oxo-2,3-dihydro-1H-benzo[d]imidazol-5-yl)piperidine-1-carboxylate (6)

To a stirred solution of tert-butyl 4-(4-amino-3-(methylamino)phenyl)piperidine-1-carboxylate (5, 60 g, 0.196 mol) in THF (900 mL) at 0° C. was added CDI (33.45 g, 0.206 mol) and the reaction mixture was stirred at RT for 16 h. Two batches were combined. The solvent was removed under reduced pressure. The residue was triturated with MTBE and filtered to afford tert-butyl 4-(3-methyl-2-oxo-2,3-dihydro-1H-benzo[d]imidazol-5-yl)piperidine-1-carboxylate (6, 88.0 g, 67.5% yield) as an off white solid. LCMS (ES+): m/z 332.3 [M+H]⁺

### Step 5: tert-butyl 4-(1-(2,6-dioxopiperidin-3-yl)-3-methyl-2-oxo-2,3-dihydro-1H-benzo[d]imidazol-5-yl)piperidine-1-carboxylate (8)

To an ice cold stirred solution of tert-butyl 4-(3-methyl-2-oxo-2,3-dihydro-1H-benzo[d]imidazol-5-yl)piperidine-1-carboxylate (6, 44 g, 0.133 mol) in anhydrous THF (900 mL) at 0° C. was added 1 M LiHMDS (403 ml. 0.387 mol). The reaction mixture was stirred for 10 min before adding 3-bromopiperidine-2,6-dione (7, 43.34 g, 0.225 mol). After addition, the reaction mixture was stirred at 70-75° C. for 16 h. Two batches were combined. The reaction mixture was cooled to (° C.) and quenched by slow addition of aqueous IN HCl (620 mL). The mixture was diluted with EtOAc (1 L) and the layers separated. The organic layer was washed with 0.5 N HCl (1.4 L), water (1.5 L×2) and brine (1.5 L). The combined organic layer was dried over sodium sulphate, filtered and concentrated under reduced pressure. The residue was purified by silica gel chromatography (20)-50% EtOAc in Petroleum ether) to obtain tert-butyl 4-(1-(2,6-dioxopiperidin-3-yl)-3-methyl-2-oxo-2,3-dihydro-1H-benzo[d]imidazol-5-yl)piperidine-1-carboxylate (8, 51.0 g, 43.4% yield) as a grey off-white solid. LCMS (ES−): m/z 441.1 [M−H]⁻

### Step 6: 3-(3-methyl-2-oxo-5-(piperidin-4-yl)-2,3-dihydro-1H-benzo[d]imidazol-1-yl)piperidine-2,6-dione (10)

To a stirred solution of tert-butyl 4-(1-(2,6-dioxopiperidin-3-yl)-3-methyl-2-oxo-2,3-dihydro-1H-benzo[d]imidazol-5-yl)piperidine-1-carboxylate (8, 25.5 g, 0.057 moles) in DCM (250 mL) at (° C.) was added TFA (87.2 ml) via dropwise addition. The reaction mixture was stirred at RT for 4 h. Two batches were combined. The volatiles were evaporated under reduced pressure and azeotroped twice with toluene. The residue was triturated with diethyl ether and dried under reduced pressure to obtain 3-(3-methyl-2-oxo-5-(piperidin-4-yl)-2,3-dihydro-1H-benzo[d]imidazol-1-yl)piperidine-2,6-dione (10, 26 g, 43.12 mmol. TFA salt) as an off white solid. LCMS (ES+): m/z 343.3 [M+H]⁺

### Step 7: tert-butyl 2-[4-[1-(2,6-dioxo-3-piperidyl)-3-methyl-2-oxo-benzimidazol-5-yl]-1-piperidyl]acetate (12)

Into a 50 mL round bottom flask containing a well-stirred solution of 3-[3-methyl-2-oxo-5-(4-piperidyl)benzimidazol-1-yl]piperidine-2,6-dione (10, 500 mg, 733.98 μmol) in DMF (5 mL) was added triethylamine (371.36 mg, 3.67 mmol, 511.51 μL). The mixture was cooled to (° C.) and tert-butyl bromoacetate (11, 186.12 mg, 954.18 μmol, 139.94 μL) was added. The reaction mixture was stirred at ambient temperature for 16 h. The reaction was quenched with water (15 mL) and the precipitate was collected by filtration, washed with water (15 mL), and dried under reduced pressure to afford tert-butyl 2-[4-[1-(2,6-dioxo-3-piperidyl)-3-methyl-2-oxo-benzimidazol-5-yl]-1-piperidyl]acetate (12, 270 mg, 560.54 μmol, 76% yield) as a pink solid. LCMS (ES+): m/z 456.9 [M+H]⁺

### Step 8: 2-(4-(1-(2,6-dioxopiperidin-3-yl)-3-methyl-2-oxo-2,3-dihydro-1H-benzo[d]imidazol-5-yl)piperidin-1-yl)acetic acid (13)

Into a 25 mL round bottom flask containing a well-stirred solution of tert-butyl 2-[4-[1-(2,6-dioxo-3-piperidyl)-3-methyl-2-oxo-benzimidazol-5-yl]-1-piperidyl]acetate (12, 300 mg, 657.13 μmol) in DCM (3 mL) was added TFA (224.78 mg, 1.97 mmol, 151.88 μL) dropwise at 0° C. The reaction mixture was stirred at ambient temperature for 3 h. The volatiles were removed under reduced pressure and the residue was triturated with diethyl ether (2×10 mL) to yield 2-(4-(1-(2,6-dioxopiperidin-3-yl)-3-methyl-2-oxo-2,3-dihydro-1H-benzo[d]imidazol-5-yl)piperidin-1-yl)acetic acid (13, 280 mg, 622.34 μmol, 95% yield) as a light brown solid. LCMS (ES+): m/z 401.3 [M+H]⁺

### 3-[5-(azetidin-3-ylamino)-3-methyl-2-oxo-benzimidazol-1-yl]piperidine-2,6-dione (10)

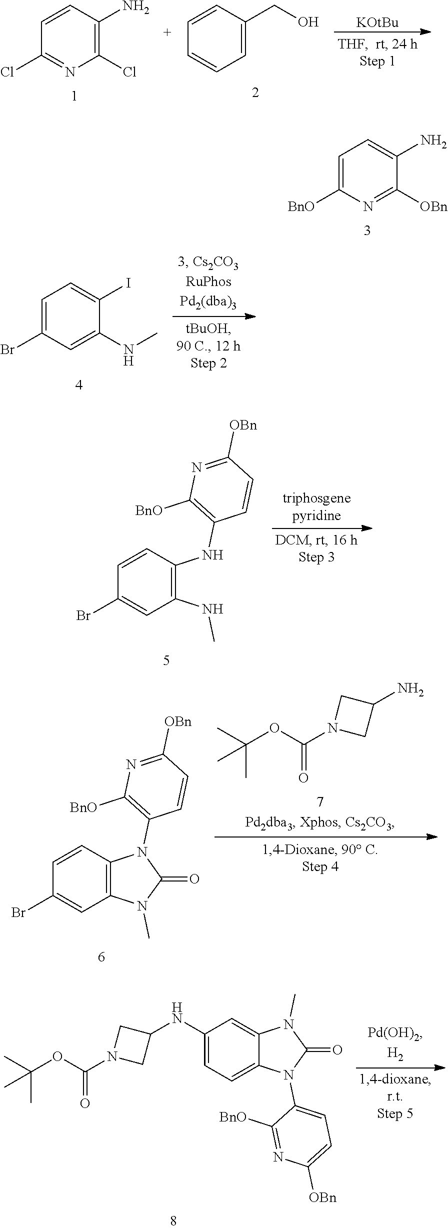

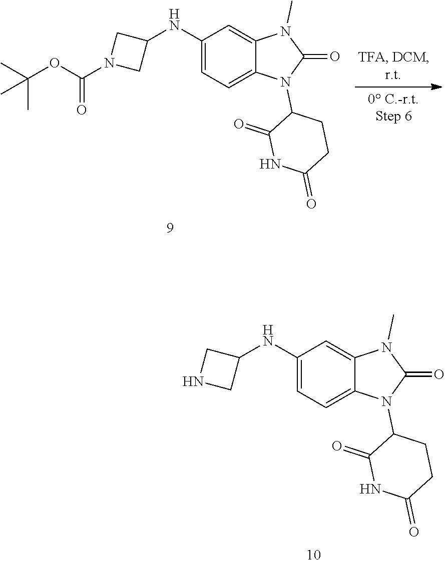 [SMILES: Cn1c(=O)n(C2CCC(=O)NC2=O)c2ccc(NC3CN(C(=O)OC(C)(C)C)C3)cc21 ; Cn1c(=O)n(C2CCC(=O)NC2=O)c2ccc(NC3CNC3)cc21]

### Step 1: 2,6-dibenzyloxypyridin-3-amine (3)

Benzyl Alcohol (2, 3.32 g, 30.67 mmol, 3.16 mL) was dissolved in THF (40 mL) and purged with nitrogen for 30 min at RT. Potassium tert-butoxide (3.44 g, 30.67 mmol) was added portion-wise over 10 min. The reaction was stirred at RT for 2 h. 2,6-dichloropyridin-3-amine (1, 2 g, 12.27 mmol) was added. The mixture was heated at reflux for 24 h. The reaction mixture was diluted with EtOAc and washed with water and brine. The organic layer was died over MgSO₄, filtered and the residue purified by silica gel chromatography (0-10% EtOAc in Hexanes) to afford 2,6-dibenzyloxypyridin-3-amine (3, 2.39 g, 7.80 mmol, 64% yield) as a dark orange oil. LCMS (ES+): m/z 306.7 [M+H]⁺

### Step 2: 4-bromo-N1-(2,6-dibenzyloxy-3-pyridyl)-N2-methyl-benzene-1,2-diamine (5)

To a stirred solution of 2,6-dibenzyloxypyridin-3-amine (3, 2.5 g, 8.16 mmol) and 5-bromo-2-iodo-N-methyl-aniline (4, 3.03 g, 8.16 mmol) in tert-butanol (30 mL) in a sealed tube was added cesium carbonate (5.32 g, 16.32 mmol). The mixture was purged with nitrogen for 10 min before dicyclohexyl-[2-(2,6-diisopropoxyphenyl)phenyl]phosphane (380.79 mg, 816.04 μmol) and Tris(dibenzylideneacetone)dipalladium(0)) (373.63 mg, 408.02 μmol) were added. The mixture was purged with nitrogen for an additional 10 min, capped, and heated at 90° C. for 12 h. The reaction mixture was cooled to RT and diluted with ethyl acetate. The mixture was washed with water and brine. The organic layer was dried over anhydrous Na₂SO₄, filtered and concentrated under reduced pressure. The residue was purified by silica gel chromatography (2-8% ethyl acetate in petroleum ether) to obtain 4-bromo-N1-(2,6-dibenzyloxy-3-pyridyl)-N2-methyl-benzene-1,2-diamine (5, 1.2 g, 1.71 mmol, 21% yield) as yellow semi solid. The material was taken on to the next step without further purification. LCMS (ES+): m/z 490.2 [M+H]⁺

### Step 3: 5-bromo-1-(2,6-dibenzyloxy-3-pyridyl)-3-methyl-benzimidazol-2-one (6)

To a stirred solution of 4-bromo-N1-(2,6-dibenzyloxy-3-pyridyl)-N2-methyl-benzene-1,2-diamine (5, 1.4 g, 2.43 mmol) in DCM (30 mL) at 0° C. was added pyridine (1.94 g, 24.27 mmol, 1.98 mL), followed by Triphosgene (1.52 g, 4.85 mmol, 95% purity). After stirring at RT for 16 h, the reaction mixture was diluted with DCM and washed with water and brine. The organic layer was dried over anhydrous sodium sulfate, filtered and concentrated under reduced pressure. The residue was purified by silica gel chromatography (0) to 40% ethyl acetate in Hexanes) to afford 5-bromo-1-(2,6-dibenzyloxy-3-pyridyl)-3-methyl-benzimidazol-2-one (6, 1.21 g, 2.33 mmol, 96% yield) as an off-white solid.

LCMS (ES+): m/z 516.2 [M+H]⁺

¹H NMR (400 MHZ, DMSO-d₆): δ 7.82-7.80 (d, J=8 Hz, 1H), 7.49-7.25 (m, 11H), 7.16-7.13 (m, 1H), 6.65-6.60 (m, 2H), 5.38-5.36 (m, 4H), 3.38 (s, 3H).

### Step 4: tert-butyl 3-[[1-(2,6-dibenzyloxy-3-pyridyl)-3-methyl-2-oxo-benzimidazol-5-yl]amino]azetidine-1-carboxylate (8)

Into a 20 mL sealed tube containing a well-stirred solution of 5-bromo-1-(2,6-dibenzyloxy-3-pyridyl)-3-methyl-benzimidazol-2-one (6, 300 mg, 0.580 mmol) in 1,4-dioxane (3 mL) were added tert-butyl 3-aminoazetidine-1-carboxylate (7, 250.14 mg, 1.45 mmol) and cesium carbonate (567.87 mg, 1.74 mmol). The reaction mixture was deoxygenated by bubbling nitrogen through for 5 min. Subsequently, tris(dibenzylideneacetone)dipalladium(0) (79.80 mg, 0.087 mmol) and XPhos (69.24 mg, 0.145 mmol) were added to the reaction mixture and the reaction mixture was heated to 90° C. for 16 h. The reaction mixture was cooled to RT and poured into water (20 mL). The aqueous layer was extracted with EtOAc (2×30 mL). Organic layers were combined, dried over anhydrous Na₂SO₄, filtered and concentrated under reduced pressure. The residue was purified by silica gel chromatography (0-100% EtOAc/petroleum ether) to obtain tert-butyl 3-[[1-(2,6-dibenzyloxy-3-pyridyl)-3-methyl-2-oxo-benzimidazol-5-yl]amino]azetidine-1-carboxylate (8, 280 mg, 0.437 mmol, 75% yield) as a pale yellow foam. LCMS (ESI): m/z 608.2 [M+H]⁺

### Step 5: tert-butyl 3-[[1-(2,6-dioxo-3-piperidyl)-3-methyl-2-oxo-benzimidazol-5-yl]amino]azetidine-1-carboxylate (9)

Into a 50 mL single neck round bottom flask containing a well-stirred solution of tert-butyl 3-[[1-(2,6-dibenzyloxy-3-pyridyl)-3-methyl-2-oxo-benzimidazol-5-yl]amino]azetidine-1-carboxylate (8, 280 mg, 0.460 mmol) in 1,4-dioxane (5 mL) was added Pd(OH)₂ (20 wt. % on carbon, 50% water) (64.71 mg, 0.460 mmol) at RT under nitrogen atmosphere. Subsequently, the reaction mixture was placed under a balloon atmosphere of hydrogen for 16 h at RT. The reaction mixture was filtered through a pad of Celite, washing with 1,4-dioxane (200 mL). The filtrate was concentrated under reduced pressure, and the residue was triturated with diethyl ether (2×25 mL) to obtain tert-butyl 3-[[1-(2,6-dioxo-3-piperidyl)-3-methyl-2-oxo-benzimidazol-5-yl]amino]azetidine-1-carboxylate (9, 160 mg, 0.219 mmol, 48% yield) as a dark-brown solid. LCMS (ESI): m/z 428.1[M−H]⁻

### Step 6: 3-[5-(azetidin-3-ylamino)-3-methyl-2-oxo-benzimidazol-1-yl]piperidine-2,6-dione (10)

Into a 25 mL single neck round bottom flask containing a well-stirred solution of tert-butyl 3-[[1-(2,6-dioxo-3-piperidyl)-3-methyl-2-oxo-benzimidazol-5-yl]amino]azetidine-1-carboxylate (9, 50 mg, 0.116 mmol) in anhydrous DCM (3 mL) was added TFA (370.00 mg, 3.25 mmol, 0.25 mL) at 0° C. The reaction mixture was allowed to warm to RT and stirred for 3 h. The reaction mixture was concentrated under reduced pressure, and the residue was azeotroped with toluene (2×5 mL) and then triturated with diethyl ether to yield 3-[5-(azetidin-3-ylamino)-3-methyl-2-oxo-benzimidazol-1-yl]piperidine-2,6-dione (10, 45 mg, 0.071 mmol, 61% yield) as a dark-brown thick gum. LCMS (ESI): m/z 330.1 [M+H]⁺.

### 3-(3-methyl-2-oxo-4-(piperidin-4-yl)-2,3-dihydro-1H-benzo[d]imidazol-1-yl)piperidine-2,6-dione (8)

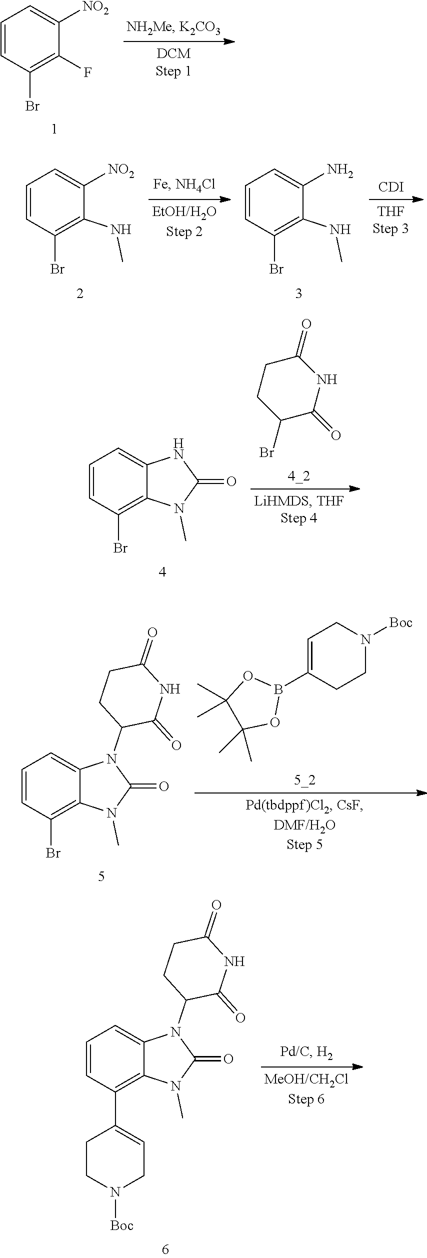

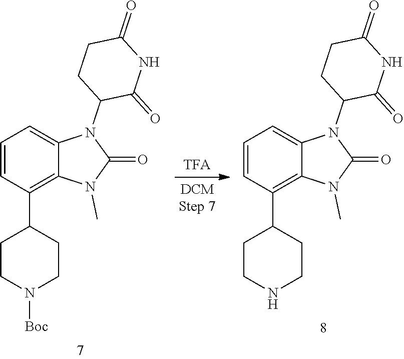 [SMILES: Cn1c(=O)n(C2CCC(=O)NC2=O)c2cccc(C3CCN(C(=O)OC(C)(C)C)CC3)c21 ; Cn1c(=O)n(C2CCC(=O)NC2=O)c2cccc(C3CCNCC3)c21]

### Step 1: 2-bromo-N-methyl-6-nitroaniline (2)

To the solution of 1-bromo-2-fluoro-3-nitrobenzene (1, 300 g, 1.36 mol) in DCM (3000 mL) was added K₂CO₃ (188.47 g, 1.36 mol). The mixture was cooled to 0° C. and MeNH₂ (2 M, 681.83 mL, 1.36 mol) was added. The mixture was stirred at 0° C. for 1 h, and then stirred at 25° C. for 3 h. The mixture was filtered and the filter cake washed with DCM (1000 mL). The filtrate was concentrated under reduced pressure to obtain 2-bromo-N-methyl-6-nitroaniline (2, 600 g, crude) as a yellow liquid, which was used for the next reaction without further purification.

### Step 2: 6-bromo-N1-methylbenzene-1,2-diamine (3)

To a solution of 2-bromo-N-methyl-6-nitroaniline (2, 200 g, 865.63 mmol) in THF (3000 mL) was added Fe (241.71 g, 4.33 mol), followed by NH₄Cl (463.04 g, 8.66 mol) in H₂O (300 mL). After stirring for 18 h at 75° C., the mixture was filtered through Celite and washed with ethyl acetate (1000 mL). The layers were separated, and the aqueous phase was extracted with ethyl acetate (500 mL). The combined organic phase was washed with brine (1000 mL), dried over anhydrous Na₂SO₄, filtered and concentrated. The residue was purified by silica gel chromatography (Petroleum ether/Ethyl acetate=50/1-5/1) to afford 6-bromo-N1-methylbenzene-1,2-diamine (3, 240 g, 1.19 mol, 46% yield) as a yellow solid.

### Step 3: 7-bromo-1-methyl-1,3-dihydro-2H-benzo[d]imidazol-2-one (4)

To a solution of 6-bromo-N1-methylbenzene-1,2-diamine (3, 240 g, 1.19 mol) in THF (2000 mL) was added CDI (967.75 g, 5.97 mol) and stirred at 60° C. for 6 h. Then the mixture was stirred at 25° C. for 12 h. The mixture was concentrated under reduced pressure and the residue triturated with ethyl acetate (1500 mL) at 25° C. for 2 h to give 7-bromo-1-methyl-1,3-dihydro-2H-benzo[d]imidazol-2-one (4, 150 g, 660.63 m mol, 55% yield) as an off-white solid. The solid was used in the next step directly without further purification.

### Step 5: 3-(4-bromo-3-methyl-2-oxo-2,3-dihydro-1H-benzo[d]imidazol-1-yl)piperidine-2,6-dione (5)

To a solution of 7-bromo-1-methyl-1,3-dihydro-2H-benzo[d]imidazol-2-one (4, 150 g, 660.62 mmol) in THF (1500 mL) was added LiHMDS (1 M in THF, 1.98 L) dropwise at 0° C. After 15 min 3-bromopiperidine-2,6-dione (4_2, 190.27 g, 990.94 mmol) in THF (300 mL) was added dropwise at 0° C. The reaction was stirred at 70° C. for 15 h. The reaction mixture was cooled to 0° C., and the reaction quenched with H₂O (1000 mL). The resulting mixture was adjusted to pH=2˜3 using 1 M aq. HCl (˜800 mL), and the precipitate collected by filtration under suction to give 3-(4-bromo-3-methyl-2-oxo-2,3-dihydro-1H-benzo[d]imidazol-1-yl)piperidine-2,6-dione (5, 65 g, 192.22 mmol, 29% yield) as a white solid.

### Step 6: tert-butyl 4-(1-(2,6-dioxopiperidin-3-yl)-3-methyl-2-oxo-2,3-dihydro-1H-benzo[d]imidazol-4-yl)-3,6-dihydropyridine-1(2H)-carboxylate (6)

To a solution of 3-(4-bromo-3-methyl-2-oxo-2,3-dihydro-1H-benzo[d]imidazol-1-yl)piperidine-2,6-dione (5, 45 g, 133.07 mmol) and tert-butyl 4-(4,4,5,5-tetramethyl-1,3,2-dioxaborolan-2-yl)-3,6-dihydro-2H-pyridine-1-carboxylate (5_2, 61.72 g, 199.61 mmol) in DMF (450 mL) and H₂O (50 mL) was added CsF (20.21 g, 133.07 mmol, 4.91 mL) and 1,1′-Bis(di-tert-butylphosphino)ferrocene]dichloropalladium(II) (17.35 g, 26.61 mmol). The mixture was stirred at 85° C. for 4 h. The mixture was added into water (1500 mL) and filtered. The filtrate was concentrated and purified by silica gel chromatography (Petroleum ether/Ethyl acetate=10/1-1/1) to afford tert-butyl 4-(1-(2,6-dioxopiperidin-3-yl)-3-methyl-2-oxo-2,3-dihydro-1H-benzo[d]imidazol-4-yl)-3,6-dihydropyridine-1(2H)-carboxy late (6, 40 g, 90.81 mmol, 68% yield) as a gray solid.

### Step 7: tert-butyl 4-(1-(2,6-dioxopiperidin-3-yl)-3-methyl-2-oxo-2,3-dihydro-1H-benzo[d]imidazol-4-yl)piperidine-1-carboxylate (7)

To a solution of tert-butyl 4-(1-(2,6-dioxopiperidin-3-yl)-3-methyl-2-oxo-2,3-dihydro-1H-benzo[d]imidazol-4-yl)-3,6-dihydropyridine-1(2H)-carboxylate (6, 40 g, 90.81 mmol) in DCM (1200 mL) and MeOH (800 mL) was added Pd/C (200 g, 10%). The mixture was stirred at 25° C. for 4 h under H₂ atmosphere (15 psi). The mixture was filtered through Celite, washing with DCM:MeOH=3:2 (1500 mL) and concentrated in vacuum. The residue was triturated with DCM (50 mL) to afford tert-butyl 4-(1-(2,6-dioxopiperidin-3-yl)-3-methyl-2-oxo-2,3-dihydro-1H-benzo[d]imidazol-4-yl)piperidine-1-carboxylate (7, 28 g, 63.28 m mol, 70% yield) as a white solid.

### Step 8: 3-(3-methyl-2-oxo-4-(piperidin-4-yl)-2,3-dihydro-1H-benzo[d]imidazol-1-yl)piperidine-2,6-dione (8)

To a solution of tert-butyl 4-(1-(2,6-dioxopiperidin-3-yl)-3-methyl-2-oxo-2,3-dihydro-1H-benzo[d]imidazol-4-yl)piperidine-1-carboxylate (7, 28 g, 63.28 mmol) in DCM (200 mL) was added TFA (123.20 g, 1.08 mol, 80.00 mL). After stirring at RT for 6 h, the mixture was concentrated and the residue triturated with MTBE (200 mL) to afford 3-(3-methyl-2-oxo-4-(piperidin-4-yl)-2,3-dihydro-1H-benzo[d]imidazol-1-yl)piperidine-2,6-dione (8, 27 g, 58.57 mmol, 93% yield, TFA salt) as an off-white solid. LCMS: m/z 343.0 [M+H]⁺

### 2-(4-(3-(2,6-dioxopiperidin-3-yl)-2-oxo-2,3-dihydrobenzo[d]oxazol-6-yl)piperidin-1-yl)acetic acid (10)

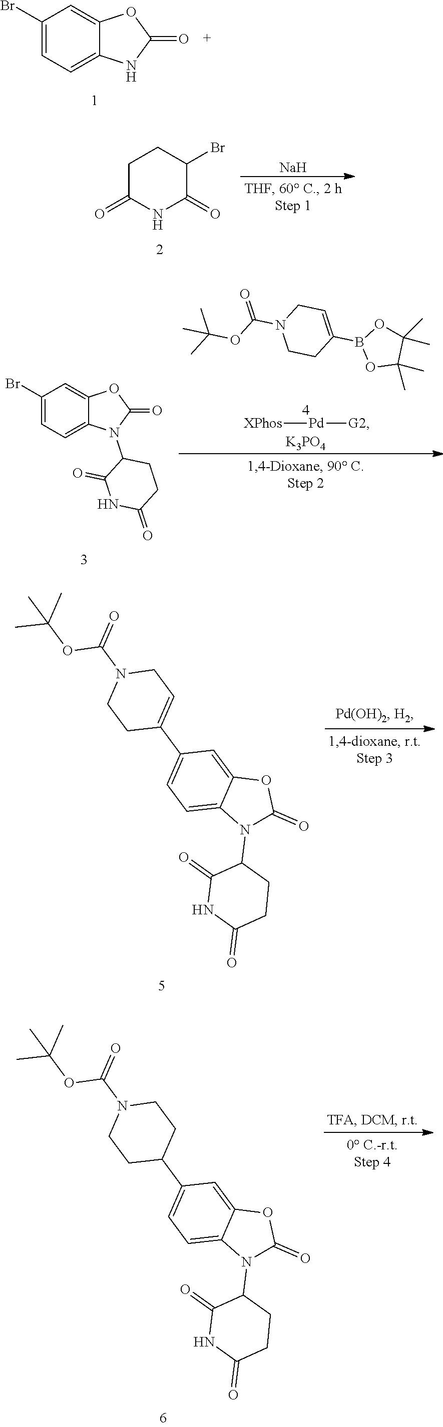

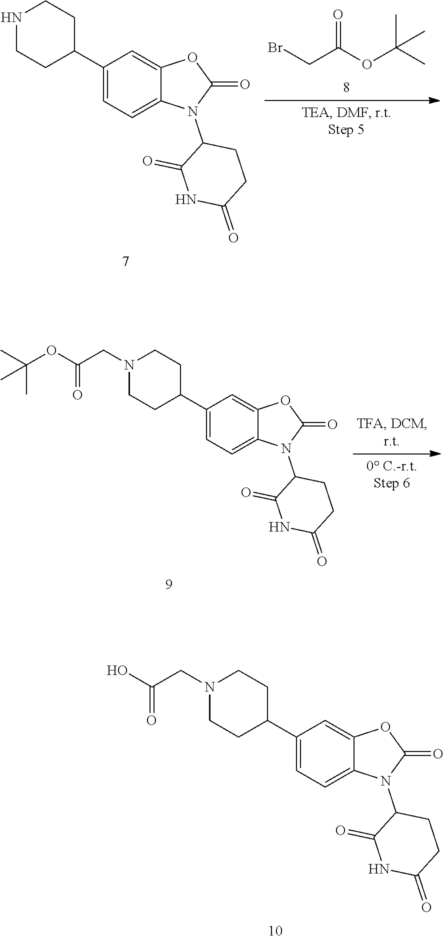

### Step 1: 3-(6-bromo-2-oxobenzo[d]oxazol-3(2H)-yl)piperidine-2,6-dione (3)

To a stirred solution of 6-bromo-3H-1,3-benzoxazol-2-one (1, 6 g, 28.04 mmol) in THF (200 mL) was added sodium hydride (60% dispersion in mineral oil) (1.29 g, 56.07 mmol) portionwise and the mixture was heated at 60° C. for 1 h. This mixture was added dropwise via cannula to a stirred solution of 3-bromopiperidine-2,6-dione (2, 8.07 g, 42.05 mmol) in THF (50 mL) at 60° C. and stirred for 2 h. The reaction was quenched with saturated ammonium chloride solution and extracted with ethyl acetate. The organics were washed with water and brine, dried over anhydrous sodium sulphate and concentrated. The residue was purified by silica gel chromatography (50% Ethyl acetate:Hexanes) to obtain 3-(6-bromo-2-oxobenzo[d]oxazol-3(2H)-yl)piperidine-2,6-dione (3, 2.9 g, 8.71 mmol, 31% yield). LCMS (ES−): m/z 323.0 [M−H]^{- 1}H NMR (400 MHZ, DMSO-D6) δ 11.23 (s, 1H), 7.73 (s, 1H), 7.43 (d, J=8.4 Hz, 1H), 7.26 (d, J=8.36 Hz, 1H), 5.41-5.37 (m, 1H), 2.87-2.84 (m, 1H), 2.71-2.64 (m, 2H), 2.18-2.15 (m, 1H)

### Step 2: (tert-butyl 4-[3-(2,6-dioxo-3-piperidyl)-2-oxo-1,3-benzoxazol-6-yl]-3,6-dihydro-2H-pyridine-1-carboxylate (5)

Into a 20 mL sealed tube containing a well-stirred solution of 3-(6-bromo-2-oxo-1,3-benzoxazol-3-yl)piperidine-2,6-dione (3, 200 mg, 0.615 mmol) and tert-butyl 4-(4,4,5,5-tetramethyl-1,3,2-dioxaborolan-2-yl)-3,6-dihydro-2H-pyridine-1-carboxylate (4, 247.28 mg, 0.800 mmol) in 1,4-dioxane (2 mL) was added anhydrous potassium phosphate tribasic (522.32 mg, 2.46 mmol) under nitrogen atmosphere. The resulting mixture was purged with nitrogen for 10 min. XPhos-Pd-G2 (48.40 mg, 0.0615 mmol) was added to the reaction mixture, and the reaction was heated to 90° C. for 16 h. The reaction mixture was cooled to RT, poured into water (10 mL) and extracted with EtOAc (2×10 mL). Organic phases were combined and washed with brine solution (10 mL). Organic layer was dried over anhydrous sodium sulfate, filtered and concentrated under reduced pressure to provide a residue, which was purified by silica-gel (230-400 mesh) flash column with 0-100% EtOAc/petroleum ether to afford tert-butyl 4-[3-(2,6-dioxo-3-piperidyl)-2-oxo-1,3-benzoxazol-6-yl]-3,6-dihydro-2H-pyridine-1-carboxylate (5, 200 mg, 0.429 mmol, 70% yield) as an off-white solid. LCMS (ESI): m/z 426.2 [M−H]⁻

### Step 3: (tert-butyl 4-[3-(2,6-dioxo-3-piperidyl)-2-oxo-1,3-benzoxazol-6-yl]piperidine-1-carboxylate (6)

Into a 50 mL single neck round bottom flask containing a well-stirred solution of tert-butyl 4-[3-(2,6-dioxo-3-piperidyl)-2-oxo-1,3-benzoxazol-6-yl]-3,6-dihydro-2H-pyridine-1-carboxylate (5, 200 mg, 0.467 mmol) in 1,4-dioxane (10 mL) was added Pd(OH)₂ (20 wt. % on carbon, 50% water) (100 mg, 0.712 mmol) at RT under nitrogen atmosphere. The resulting suspension was stirred at RT under an atmosphere of hydrogen for 16 h. The reaction mixture was filtered through a pad of Celite, washing with 1:1 EtOAc/DCM (200 mL). The filtrate was concentrated under reduced pressure to provide a residue which was triturated with Et₂O (2×15 mL) to afford tert-butyl 4-[3-(2,6-dioxo-3-piperidyl)-2-oxo-1,3-benzoxazol-6-yl]piperidine-1-carboxylate (6, 190 mg, 0.384 mmol, 82% yield) as an off-white solid. LCMS (ESI): m/z 427.9 [M−H]⁻

### Step 4: (3-[2-oxo-6-(4-piperidyl)-1,3-benzoxazol-3-yl]piperidine-2,6-dione (7)

Into a 50 mL single neck round bottom flask containing a well-stirred solution of tert-butyl 4-[3-(2,6-dioxo-3-piperidyl)-2-oxo-1,3-benzoxazol-6-yl]piperidine-1-carboxylate (6, 280 mg, 0.651 mmol) in DCM (3 mL) was added trifluoroacetic acid (3.38 g, 29.63 mmol, 2.28 mL) at RT, and the resulting reaction mixture was stirred for 3 h. The reaction mixture was concentrated under reduced pressure and azeotroped with toluene (2×20 mL). The residue was triturated with diethyl ether (2×10 mL) to afford 3-[2-oxo-6-(4-piperidyl)-1,3-benzoxazol-3-yl]piperidine-2,6-dione (7, 170 mg, 0.303 mmol, 47% yield, TFA salt) as a grey-coloured foam which was used in the next step without further purification. LCMS (ESI): m/z 330.2 [M+H]⁺

### Step 5: (tert-butyl 2-[4-[3-(2,6-dioxo-3-piperidyl)-2-oxo-1,3-benzoxazol-6-yl]-1-piperidyl]acetate (9)

Into a 25 mL single neck round bottom flask containing a well-stirred solution of 3-[2-oxo-6-(4-piperidyl)-1,3-benzoxazol-3-yl]piperidine-2,6-dione (7, 150 mg, 0.455 mmol) in DMF (5 mL) were added triethylamine (230.43 mg, 2.28 mmol, 317.40 μL) and tert-butyl bromoacetate (8, 102.16 mg, 0.523 mmol, 76.81 μL) at RT, and the resulting mixture was stirred for 16 h. The reaction mixture was poured into ice cold water (20 mL). Precipitation was removed by filtration, and the filtrate was concentrated under reduced pressure to yield tert-butyl 2-[4-[3-(2,6-dioxo-3-piperidyl)-2-oxo-1,3-benzoxazol-6-yl]-1-piperidyl]acetate (9, 150 mg, 0.305 mmol, 67% yield) as an off-white solid, which was taken to the next step without further purification. LCMS (ESI): m/z 444.0 [M+H]⁺

### Step 6: (2-[4-[3-(2,6-dioxo-3-piperidyl)-2-oxo-1,3-benzoxazol-6-yl]-1-piperidyl]acetic acid (10)

Into a 50 mL single neck round bottom flask containing a well-stirred solution of tert-butyl 2-[4-[3-(2,6-dioxo-3-piperidyl)-2-oxo-1,3-benzoxazol-6-yl]-1-piperidyl]acetate (9, 190 mg, 0.428 mmol) in DCM (3 mL) was added trifluoroacetic acid (2.22 g, 19.47 mmol, 1.5 mL) at RT, and the resulting mixture was stirred for 6 h. The reaction mixture was concentrated under reduced pressure to provide a residue, which was azeotroped with toluene (2×20 mL) and triturated with diethyl ether (2×10 mL) to afford 2-[4-[3-(2,6-dioxo-3-piperidyl)-2-oxo-1,3-benzoxazol-6-yl]-1-piperidyl]acetic acid (10, 150 mg, 0.234 mmol, 55% yield) as a grey-coloured foam which was used without further purification. LCMS (ESI): m/z 387.9 [M+H]⁺

### 2-[4-[1-(2,6-dioxo-3-piperidyl)-3-isopropyl-2-oxo-benzimidazol-5-yl]-1-piperidyl]acetic acid (14)

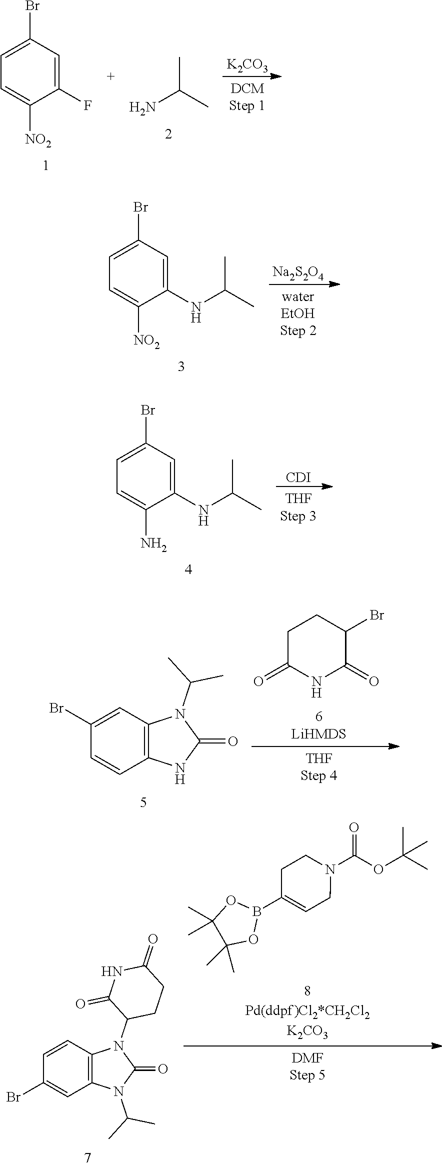

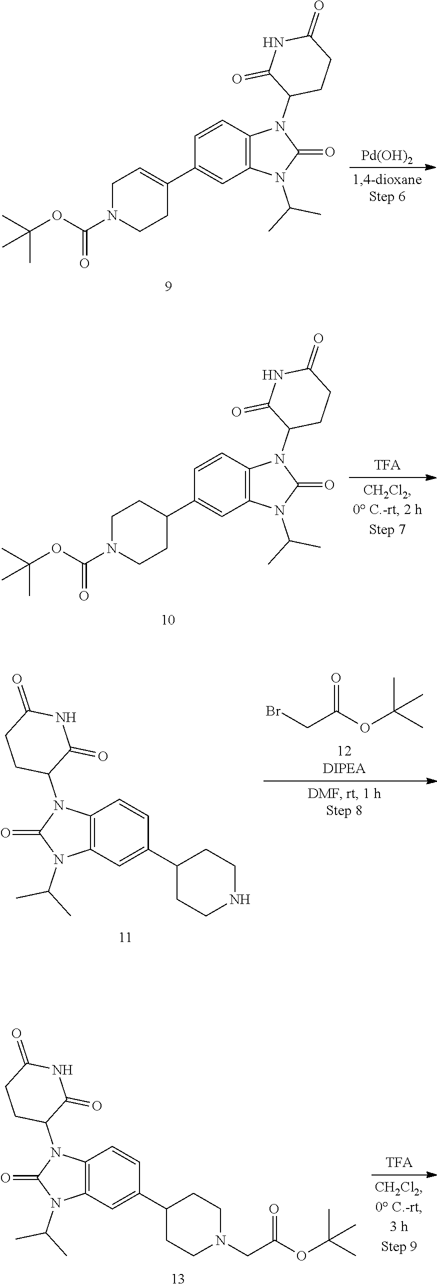

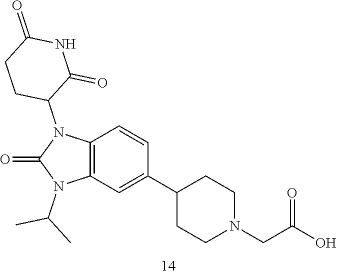 [SMILES: CC(C)n1c(=O)n(C2CCC(=O)NC2=O)c2ccc(C3CCN(CC(=O)O)CC3)cc21]

### Step 1: 5-bromo-N-isopropyl-2-nitro-aniline (3)

To a solution of 4-bromo-2-fluoro-1-nitro-benzene (1, 2.4 g, 10.91 mmol) in DCM (25 mL) was added isopropylamine (2, 644.9 mg, 10.91 mmol, 0.933 mL) and potassium carbonate (3.02 g, 21.8 mmol). After 16 h, the reaction was diluted with DCM (50 mL) and washed with water (3×40 mL). The organic layer was dried over sodium sulfate, filtered and concentrated to obtain 5-bromo-N-isopropyl-2-nitro-aniline (3, 2.8 g, 10.78 mmol, 98.8% yield) as a bright yellow solid. The material was used in the next step without purification. LCMS (ES+): m/z 261 [M+H]⁺

### Step 2: 4-bromo-N2-isopropyl-benzene-1,2-diamine (4)

To a solution of 5-bromo-N-isopropyl-2-nitro-aniline (3, 2.8 g, 10.81 mmol) in ethanol (60 mL) was added a solution of sodium dithionite (8.47 g, 48.63 mmol) in water (25 mL). After 3 h, the reaction mixture was concentrated under reduced pressure. The residue was taken up in ethyl acetate (50 mL) and washed with water (3×40 mL). The organic layer was dried over sodium sulfate, filtered and concentrated to obtain 5-bromo-N¹-isopropyl-benzene-1,2-diamine (4, 2.2 g, 8.41 mmol, 78% yield) as a pale yellow liquid. The material was used in the next step without further purification. LCMS (ES−): m/z 229 [M−H]⁻

### Step 3: 5-bromo-3-isopropyl-1H-benzimidazol-2-one (5)

To a solution of 5-bromo-N¹-isopropyl-benzene-1,2-diamine (4, 2.2 g, 9.60 mmol) in THF (25 mL) was added CDI (2.34 g, 14.40 mmol). After 16 h, the mixture was diluted with Ethyl Acetate (50) mL) and was washed with Water (3×40 mL). The organic layer was dried with Sodium Sulphate, filtered and concentrated. The residue was purified by silica gel chromatography (40% EtOAc:Hex) to obtain 5-bromo-3-isopropyl-1H-benzimidazol-2-one (5, 1.48 g, 5.80 mmol, 60% yield) as a white solid. LCMS (ES+): m/z 257 [M+H]⁺

### Step 4: 3-(5-bromo-3-isopropyl-2-oxo-benzimidazol-1-yl)piperidine-2,6-dione (7)

A solution of 5-bromo-3-isopropyl-1H-benzimidazol-2-one (5, 1.5 g, 6.20 mmol) in THF (20 mL) was cooled to 0° C.) and Lithium bis(trimethylsilyl)amide (1.04 g, 6.20 mmol) was added dropwise. After 15 min, a solution of 3-bromopiperidine-2,6-dione (6, 1.19 g, 6.20 mmol) in THF (1.5 mL) was added. The reaction mixture was allowed to come to RT and then heated at 60° C. for 4 h. The mixture was cooled to RT and quenched with 1.5 N HCl solution. The reaction mixture was diluted with EtOAc (20 mL) and washed with 1.5 N HCl (3×25 mL). The organic layer was dried with Sodium Sulphate, filtered and concentrated under reduced pressure. The residue was purified via flash column chromatography (50% EtOAc:Hex) to obtain 3-(5-bromo-3-isopropyl-2-oxo-benzimidazol-1-yl)piperidine-2,6-dione (7, 480 mg, 1.31 mmol, 21% yield) as an off-white solid. LCMS (ES+): m/z 366 [M+H]⁺

### Step 5: tert-butyl 4-[1-(2,6-dioxo-3-piperidyl)-3-isopropyl-2-oxo-benzimidazol-5-yl]-3,6-dihydro-2H-pyridine-1-carboxylate (9)

In a sealed tube, a solution of 3-(5-bromo-3-isopropyl-2-oxo-benzimidazol-1-yl)piperidine-2,6-dione (7, 1.3 g, 3.55 mmol) and tert-butyl 4-(4,4,5,5-tetramethyl-1,3,2-dioxaborolan-2-yl)-3,6-dihydropyridine-1(2H)-carboxylate (8, 2.20 g, 7.10 mmol) in DMF (15 mL) was purged with N₂ gas for 10 min. Subsequently, potassium carbonate (1.47 g, 10.65 mmol) and [1,1′-Bis(diphenylphosphino)ferrocene]dichloropalladium(II) dichloromethane complex (289.9 mg, 354.99 μmol) were added. After complete addition, the tube was sealed and heated to 70° C. After 16 h, the reaction was cooled to RT, extracted with EtOAc (75 mL) and washed with water (3×20 mL). The organic layer was dried with sodium sulphate, filtered and concentrated under reduced pressure to obtain tert-butyl 4-[1-(2,6-dioxo-3-piperidyl)-3-isopropyl-2-oxo-benzimidazol-5-yl]-3,6-dihydro-2H-pyridine-1-carboxylate (9, 1.02 g, 1.72 mmol, 48% yield) as an off white solid. The material was used directly in the next step. LCMS (ES+): m/z 469.2 [M+H]⁺

### Step 6: tert-butyl 4-[1-(2,6-dioxo-3-piperidyl)-3-isopropyl-2-oxo-benzimidazol-5-yl]piperidine-1-carboxylate (10)

To a solution of tert-butyl 4-[1-(2,6-dioxo-3-piperidyl)-3-isopropyl-2-oxo-benzimidazol-5-yl]-3,6-dihydro-2H-pyridine-1-carboxylate (9, 350 mg, 746.99 μmol) in 1,4-dioxane (5 mL) was added palladium hydroxide on carbon (10 wt. % 50% water) (209.81 mg, 1.49 mmol). The reaction was placed under a hydrogen balloon atmosphere. After 16 h, the reaction was filtered through Celite. The filtrate was concentrated and the residue purified by silica gel chromatography (70% EtOAc:Hex) to obtain tert-butyl 4-[1-(2,6-dioxo-3-piperidyl)-3-isopropyl-2-oxo-benzimidazol-5-yl]piperidine-1-carboxylate (10, 345 mg, 554.29 μmol, 74% yield) as a white solid. LCMS (ES−): m/z 469 [M−H]⁻

### Step 7: 3-[3-isopropyl-2-oxo-5-(4-piperidyl)benzimidazol-1-yl]piperidine-2,6-dione (11)

Into a 25 mL single neck round bottom flask containing a well-stirred solution of tert-butyl 4-[1-(2,6-dioxo-3-piperidyl)-3-isopropyl-2-oxo-benzimidazol-5-yl]piperidine-1-carboxylate (10, 500 mg, 1.06 mmol) in anhydrous DCM (5 mL) was added TFA (1.48 g, 12.98 mmol, 1 mL) at 0° C.) The reaction was stirred for 2 h at ambient temperature. The volatiles were removed under reduced pressure and the residue azeotroped with toluene (2×15 mL), then triturated with diethyl ether (20) mL) to obtain 3-[3-isopropyl-2-oxo-5-(4-piperidyl)benzimidazol-1-yl]piperidine-2,6-dione (11, 505 mg, 1.03 mmol, 97% yield, TFA salt) as an off-white solid. LCMS (ES+): m/z 371.0 [M+H]⁺

### Step 8: tert-butyl 2-[4-[1-(2,6-dioxo-3-piperidyl)-3-isopropyl-2-oxo-benzimidazol-5-yl]-1-piperidyl]acetate (13)

Into a 25 mL single neck round bottom flask containing a well-stirred solution of 3-[3-isopropyl-2-oxo-5-(4-piperidyl)benzimidazol-1-yl]piperidine-2,6-dione (11, 500 mg, 1.35 mmol) in anhydrous DMF (5 mL) were added DIPEA (523.33 mg, 4.05 mmol, 705.30 μL) and tert-butyl bromoacetate (12, 289.60 mg, 1.48 mmol, 217.74 μL). After 2 h, the reaction mixture was poured into ice-cold water (20 mL) and the aqueous layer was extracted with DCM (2×25 mL). The organic layers were combined, washed with brine (15 mL), dried over anhydrous Na₂SO₄ and filtered. The residue was triturated with diethyl ether, filtered and dried to afford tert-butyl 2-[4-[1-(2,6-dioxo-3-piperidyl)-3-isopropyl-2-oxo-benzimidazol-5-yl]-1-piperidyl]acetate (13, 500 mg, 829.88 μmol, 61% yield) as an off-white solid. LCMS (ES+): m/z 485.0 [M+H]⁺

### Step 9: 2-[4-[1-(2,6-dioxo-3-piperidyl)-3-isopropyl-2-oxo-benzimidazol-5-yl]-1-piperidyl]acetic acid (14)

Into a 25 mL single neck round bottom flask containing a well stirred solution of tert-butyl 2-[4-[1-(2,6-dioxo-3-piperidyl)-3-isopropyl-2-oxo-benzimidazol-5-yl]-1-piperidyl] acetate (13, 500 mg, 1.03 mmol) in anhydrous DCM (5 mL) was added TFA (1.48 g, 12.98 mmol, 1 mL) at 0° C. The mixture was stirred at RT for 3 h. The reaction mixture was concentrated under reduced pressure, azeotroped with toluene (2×15 mL), then triturated with diethyl ether (20 mL) to obtain 2-[4-[1-(2,6-dioxo-3-piperidyl)-3-isopropyl-2-oxo-benzimidazol-5-yl]-1-piperidyl] acetic acid (14, 440 mg, 656.95 μmol, 64% yield, TFA salt) as an off-white solid. LCMS (ES+): m/z 428.9 [M+H]⁺

### tert-butyl 4-(3-(2,6-dioxopiperidin-3-yl)-1-methyl-1H-indazol-6-yl)piperidine-1-carboxylate (6)

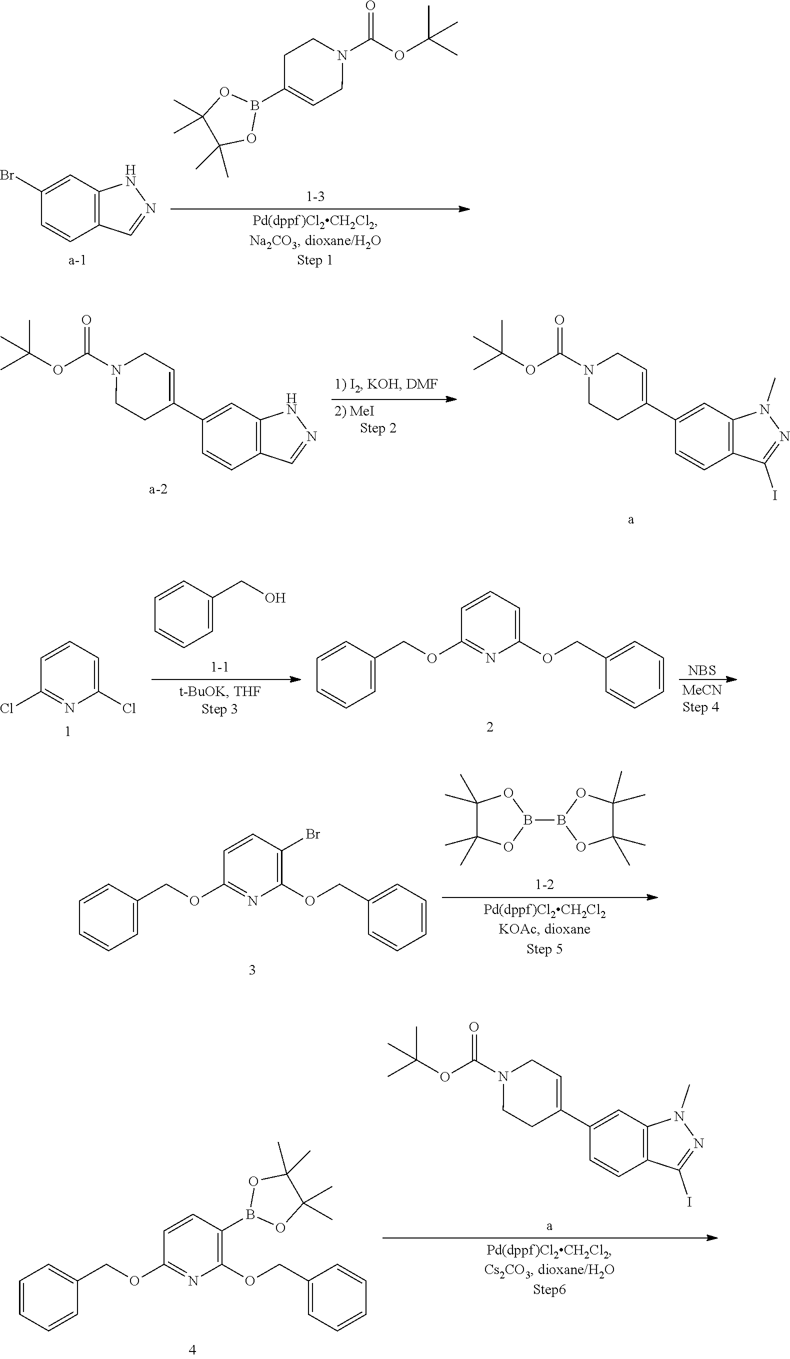

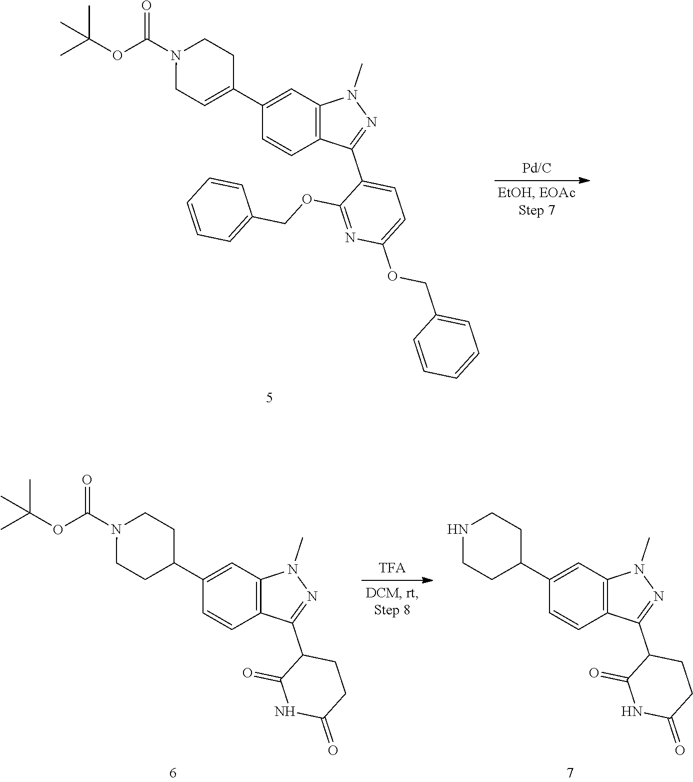 [SMILES: Cn1nc(-c2ccc(OCc3ccccc3)nc2OCc2ccccc2)c2ccc(C3=CCN(C(=O)OC(C)(C)C)CC3)cc21 ; Cn1nc(C2CCC(=O)NC2=O)c2ccc(C3CCN(C(=O)OC(C)(C)C)CC3)cc21 ; Cn1nc(C2CCC(=O)NC2=O)c2ccc(C3CCNCC3)cc21]

### Step 1: tert-butyl 4-(1H-indazol-6-yl)-3,6-dihydropyridine-1(2H)-carboxylate (a-2)

A mixture of compound 6-bromo-1H-indazole (a-1, 57.0 g, 289 mmol), tert-butyl 4-(4,4,5,5-tetramethyl-1,3,2-dioxaborolan-2-yl)-3,6-dihydropyridine-1(2H)-carboxylate (1-3, 134 g, 433 mmol), Pd(dppf)Cl₂·CH₂Cl₂ (12.0 g, 14.6 mmol) and Na₂CO₃ (100 g, 943 mmol) in dioxane (480 mL) and H₂O (120 mL) was stirred at 105° C. for 12 h. The mixture was filtered through a pad of Celite and washed with ethyl acetate (500 mL). The filtrate was washed with brine (150 mL×3), dried over Na₂SO₄, filtered and concentrated under reduced pressure. The residue was purified by silica gel chromatography (0˜30% ethyl acetate/petroleum ether) to afford tert-butyl 4-(1H-indazol-6-yl)-3,6-dihydropyridine-1(2H)-carboxylate (a-2, 80.0 g, 239 mmol, 83% yield) as yellow oil. LCMS: m/z 300.1 [M+H]⁺

### Step 2: tert-butyl 4-(3-iodo-1-methyl-1H-indazol-6-yl)-3,6-dihydropyridine-1(2H)-carboxylate (a)

To a solution of tert-butyl 4-(1H-indazol-6-yl)-3,6-dihydropyridine-1(2H)-carboxylate (a-2, 75.0 g, 224 mmol) in DMF (700 mL) was added KOH (37.7 g, 672 mmol) and I₂ (85.3 g, 336 mmol, 67.7 mL). The mixture was stirred at 25° C. for 12 h and was cooled to 0° C. MeI (44.6 g, 314 mmol, 19.6 mL) was then added. The resulting mixture was stirred at 25° C. for 1 h. The mixture was poured into water (1500 mL) and extracted with ethyl acetate (500 mL×3). The combined organic phase was washed by brine (500 mL×3) and dried over Na₂SO₄, filtered and concentrated under vacuum to give a residue, which was purified by silica gel chromatography (0˜8% ethyl acetate/petroleum ether) to obtain tert-butyl 4-(3-iodo-1-methyl-1H-indazol-6-yl)-3,6-dihydropyridine-1(2H)-carboxylate (a, 23.0 g, 52.3 mmol, 23% yield) as a yellow oil. LCMS: m/z 440.1 [M+H]⁺

### Step 3: 2,6-bis(benzyloxy)pyridine (2)

To a solution of t-BuOK (190 g, 1.69 mol) in THF (1.00 L) was added phenylmethanol (1-1, 73.4 g, 679 mmol, 70.6 mL) at 0° C. 2,6-Dichloropyridine (1, 50.0 g, 338 mmol) was added to the mixture at 25° C. and stirred at 75° C. for 12 h. The reaction was quenched with sat. aq. NH₄Cl (200 mL) at 0° C., diluted with ethyl acetate (200 mL), and extracted with ethyl acetate (200 mL×3). The combined organic layers were washed with brine (500 mL), dried over Na₂SO₄, filtered, and concentrated under reduced pressure to give a residue. The residue was triturated with petroleum ether (150 mL) to afford 2,6-bis(benzyloxy)pyridine (2, 84.0 g, 85% yield) as a yellow solid. LCMS: m/z 292.2 [M+H]⁺

### Step 4: 2,6-bis(benzyloxy)-3-bromopyridine (3)

To a solution of 2,6-bis(benzyloxy)pyridine (2, 34.0 g, 116 mmol) in MeCN (100 mL) was added a solution of NBS (21.0 g, 118 mmol, 1.01 eq) in MeCN (200 mL) at 40° C. and the reaction mixture was stirred at 85° C. for 12 h. The reaction mixture was concentrated under reduced pressure, diluted with water (500 mL), and extracted with ethyl acetate (300 mL×3). The combined organic layers were washed with brine (200 mL), dried over Na₂SO₄, filtered and concentrated, and the residue triturated with petroleum ether (60 ml) to afford 2,6-bis(benzyloxy)-3-bromopyridine (3, 27.7 g, 64% yield). LCMS: m/z 371.9 [M+H]⁺

### Step 5: 2,6-bis(benzyloxy)-3-(4,4,5,5-tetramethyl-1,3,2-dioxaborolan-2-yl)pyridine (4)

To a solution of 2,6-bis(benzyloxy)-3-bromopyridine (3, 52.4 g, 139 mmol) in dioxane (500 mL) was added 4,4,4′,4′,5,5,5′,5′-octamethyl-2,2′-bi(1,3,2-dioxaborolane) (1-2, 37.1 g, 146 mmol), KOAc (41.0 g, 418 mmol) and Pd(dppf)Cl₂·CH₂Cl₂ (5.69 g, 6.97 mmol). The reaction mixture was stirred at 105° C. for 12 h. The reaction mixture was filtered through a pad of Celite. The filtrate was diluted with water (500 mL) and extracted with ethyl acetate (500 mL×2). The extracts were washed with brine (400 mL), dried over Na₂SO₄, filtered and concentrated. The residue was purified by silica gel chromatography (0)˜100% ethyl acetate/petroleum ether) to afford 2,6-bis(benzyloxy)-3-(4,4,5,5-tetramethyl-1,3,2-dioxaborolan-2-yl)pyridine (4, 35.0 g, 60.1% yield) as yellow oil. LCMS: m/z 418.3 [M+H]⁺

### Step 6: tert-butyl 4-(3-(2,6-bis(benzyloxy)pyridin-3-yl)-1-methyl-1H-indazol-6-yl)-3,6-dihydropyridine-1(2H)-carboxylate (5)

To a solution of 2,6-bis(benzyloxy)-3-(4,4,5,5-tetramethyl-1,3,2-dioxaborolan-2-yl)pyridine (4, 20.0 g, 45.53 mmol), tert-butyl 4-(1H-indazol-6-yl)-3,6-dihydropyridine-1(2H)-carboxylate (a, 26.6 g, 63.7 mmol) and Cs₂CO₃ (44.5 g, 136 mmol) in dioxane (200 mL) and H₂O (40 mL) was added Pd(dppf)Cl₂·CH₂Cl₂ (3.72 g, 4.55 mmol, 0.10 eq). The reaction mixture was stirred at 100° C. for 2 h. The reaction mixture was filtered through a pad of Celite, and the filtrate was washed with brine (60) mL×3 mL), dried over Na₂SO₄, filtered and concentrated. The residue was purified by silica gel chromatography (0)˜100% ethyl acetate/petroleum ether) to obtain tert-butyl 4-(3-(2,6-bis(benzyloxy)pyridin-3-yl)-1-methyl-1H-indazol-6-yl)-3,6-dihydropyridine-1 (2H)-carboxylate (5, 20.0 g, 73% yield) as yellow oil. LCMS: m/z 603.3 [M+H]⁺

### Step 7: tert-butyl 4-(3-(2,6-dioxopiperidin-3-yl)-1-methyl-1H-indazol-6-yl)piperidine-1-carboxylate (6)

To a solution of tert-butyl 4-(3-(2,6-bis(benzyloxy)pyridin-3-yl)-1-methyl-1H-indazol-6-yl)-3,6-dihydropyridine-1(2H)-carboxylate (5, 18.0 g, 29.8 mmol, 1.00 eq) in EtOH (270 mL) and EtOAc (270) mL) was added Pd/C (4.00 g, 10% purity) under N₂ atmosphere. The suspension was degassed and purged with H₂ 3 times. The mixture was stirred under H₂ (15 psi) at 30° C. for 24 h. The reaction mixture was filtered through a pad of Celite and the filtrate was concentrated. The residue was purified by silica gel chromatography (ethyl acetate/petroleum ether) to afford tert-butyl 4-(3-(2,6-dioxopiperidin-3-yl)-1-methyl-1H-indazol-6-yl)piperidine-1-carboxylate (6, 5.3 g. 41% yield) as a white solid. LCMS: m/z 427.2 [M+H]⁺

### Step 8: 3-[1-methyl-6-(4-piperidyl)indazol-3-yl]piperidine-2,6-dione (7)

Into a 25 mL single neck round bottom flask containing a well-stirred solution of tert-butyl 4-[3-(2,6-dioxo-3-piperidyl)-1-methyl-indazol-6-yl]piperidine-1-carboxylate (6, 500 mg, 1.17 mmol) in anhydrous DCM (5 mL) was added TFA (668.35 mg, 5.86 mmol, 451.59 μL) at 0° C. After stirring at RT for 3 h, the reaction mixture was concentrated under reduced pressure. The residue was azeotroped with toluene (2×15 mL) and triturated with diethylether (20 mL) to afford 3-[1-methyl-6-(4-piperidyl)indazol-3-yl]piperidine-2,6-dione (7, 500 mg, 1.12 mmol, 95% yield, TFA salt) as an off-white solid. LCMS (ESI) m/z 326.9 [M+H]⁺

### 3-[3-methyl-2-oxo-4-(4-piperidyloxy)benzimidazol-1-yl]piperidine-2,6-dione (11)

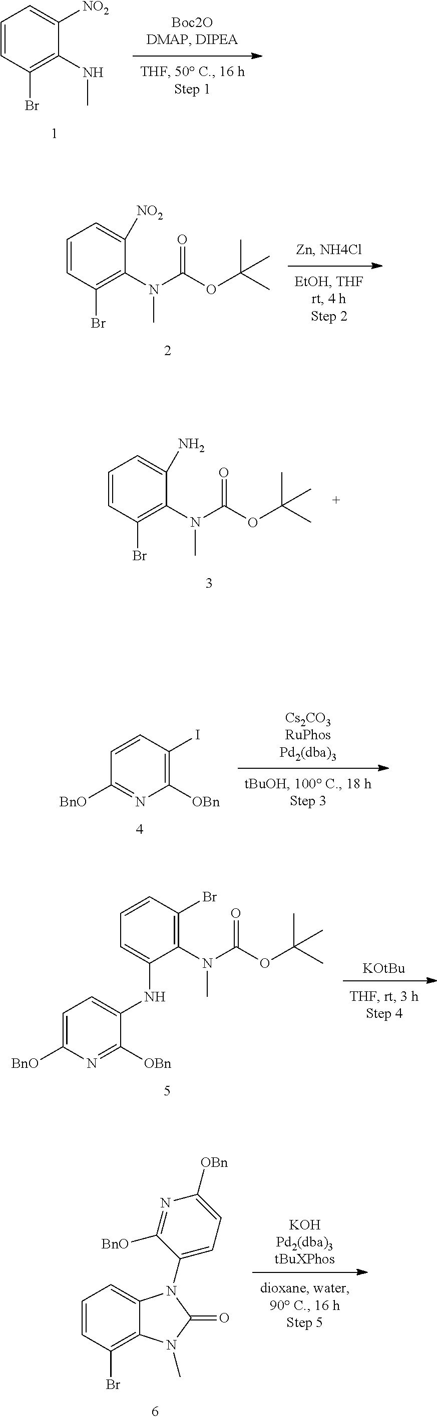

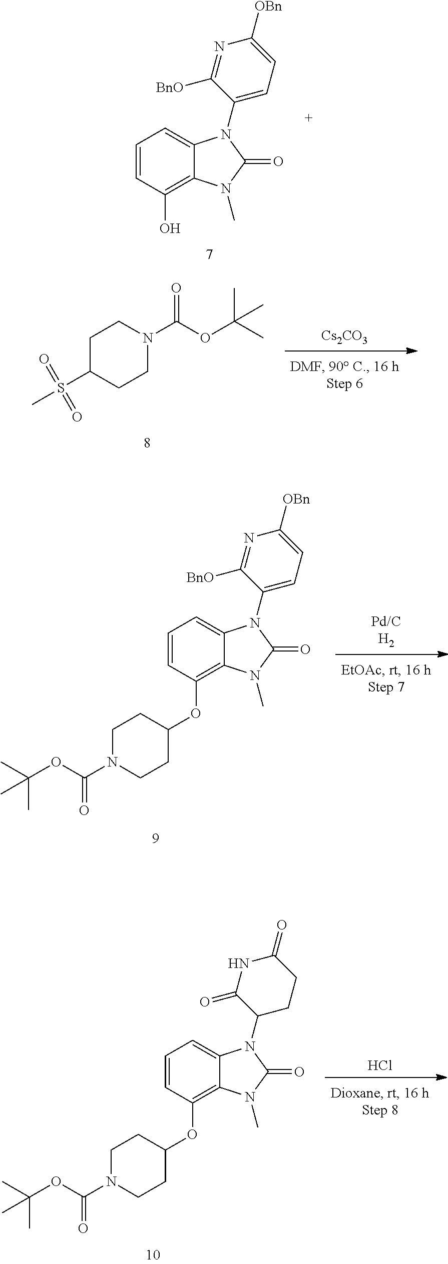

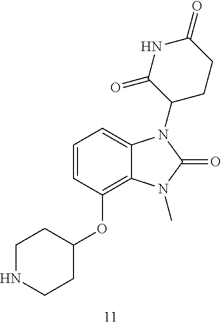 [SMILES: Cn1c(=O)n(C2CCC(=O)NC2=O)c2cccc(OC3CCNCC3)c21]

### Step 1: tert-butyl N-(2-bromo-6-nitro-phenyl)-N-methyl-carbamate (2)

To the solution of 2-bromo-N-methyl-6-nitro-aniline (1, 6 g, 25.97 mmol) in THF (50 mL) was added N,N-dimethylpyridin-4-amine (4.76 g, 38.95 mmol), tert-Butoxycarbonyl tert-butyl carbonate (22.67 g, 103.88 mmol, 23.84 mL) and diisopropyl ethylamine (10.07 g, 77.91 mmol, 13.57 mL) at 0° C., and the reaction was heated at 50° C. for 16 h. The reaction was diluted with ethyl acetate and washed with water and brine solution. The organic layer was dried over sodium sulphate and concentrated. The residue was purified via silica gel column chromatography to obtain tert-butyl N-(2-bromo-6-nitro-phenyl)-N-methyl-carbamate (2, 3 g, 8.88 mmol, 34% yield). LCMS: m/z 331.2 [M+H]⁺

### Step 2: tert-butyl N-(2-amino-6-bromo-phenyl)-N-methyl-carbamate (3)

To a stirred solution of tert-butyl N-(2-bromo-6-nitro-phenyl)-N-methyl-carbamate (2, 4 g, 12.08 mmol) in ethanol (10 mL) and THF (10 mL) were added ammonium chloride (6.46 g, 120.79 mmol, 4.22 mL) and zinc (7.90 g, 120.79 mmol, 1.11 mL). The resulting mixture was stirred at RT for 4 h. The reaction mixture was filtered through Celite and washed with ethanol. The filtrate was concentrated under reduced pressure and the residue purified by column chromatography (35% ethyl acetate-hexane) to afford tert-butyl N-(2-amino-6-bromo-phenyl)-N-methyl-carbamate (3, 2.54 g, 7.76 mmol, 64% yield). LCMS: m/z 300.9 [M+H]⁺

### Step 3: tert-butyl N-[2-bromo-6-[(2,6-dibenzyloxy-3-pyridyl)amino]phenyl]-N-methyl-carbamate (5)

To a stirred solution of tert-butyl N-(2-amino-6-bromo-phenyl)-N-methyl-carbamate (3, 3.8 g, 12.62 mmol) and 2,6-dibenzyloxy-3-iodo-pyridine (4, 6.32 g, 15.14 mmol) in t-Butanol (60 mL), was added cesium carbonate (12.33 g, 37.85 mmol). The resulting mixture was purged with argon and 2-dicyclohexylphosphino-2,6-diidopropoxy-1,1-biphenyl (588.76 mg, 1.26 mmol) and Tris(dibenzylideneacetone)dipalladium(0) (577.68 mg, 630.85 μmol) were added. The resulting mixture was heated at 100° C. for 18 h. The reaction mixture was diluted with ethyl acetate, filtered through Celite and washed with ethyl acetate. The filtrate was washed with water and brine, dried over anhydrous sodium sulphate, filtered and concentrated. The residue was purified by silica gel chromatography to afford tert-butyl N-[2-bromo-6-[(2,6-dibenzyloxy-3-pyridyl)amino]phenyl]-N-methyl-carbamate (5, 1.8 g, 3.05 mmol, 24% yield) and 4-bromo-1-(2,6-dibenzyloxy-3-pyridyl)-3-methyl-benzimidazol-2-one (6, 360 mg, 697.15 μmol, 6% yield). LCMS: m/z 590.2 [M+H]⁺

### Step 4: 4-bromo-1-(2,6-dibenzyloxy-3-pyridyl)-3-methyl-benzimidazol-2-one (6)

To a stirred solution of tert-butyl N-[2-bromo-6-[(2,6-dibenzyloxy-3-pyridyl)amino]phenyl]-N-methyl-carbamate (5, 1.4 g, 2.37 mmol) in THF (20 mL) was added potassium tert-butoxide (319.25 mg, 2.85 mmol). The resulting mixture was stirred at RT for 3 h. The reaction mixture was concentrated, then diluted with ethyl acetate and washed with water and brine. The organic layer was dried over anhydrous sodium sulphate, filtered and concentrated. The residue was purified by silica gel chromatography (30% ethyl acetate-hexane) to afford 4-bromo-1-(2,6-dibenzyloxy-3-pyridyl)-3-methyl-benzimidazol-2-one (6, 0.92 g, 1.76 mmol, 74% yield).

LCMS: m/z 516.0 [M+H]^{+ 1}H NMR (400 MHZ, DMSO-d₆) δ 7.82 (d, J=8.2 Hz, 1H), 7.45-7.32 (m, 5H), 7.27-7.24 (m, 6H), 6.92 (t, J=8.0 Hz, 1H), 6.68 (d, J=7.7 Hz, 1H), 6.62 (d, J=8.3 Hz, 1H), 5.43-5.33 (m, 4H), 3.68 (s, 3H).

### Step 5: 1-(2,6-dibenzyloxy-3-pyridyl)-4-hydroxy-3-methyl-benzimidazol-2-one (7)

To a stirred solution of 4-bromo-1-(2,6-dibenzyloxy-3-pyridyl)-3-methyl-benzimidazol-2-one (6, 1.05 g, 2.03 mmol) in dioxane (8 mL) and water (8 mL) was added potassium hydroxide (250.98 mg, 4.47 mmol, 123.03 μL). The mixture was purged with argon and Tris(dibenzylideneacetone)dipalladium(0)) (186.20 mg, 203.34 μmol) and tBuXPhos (345.38 mg. 813.35 μmol) were added. The resulting mixture was heated at 90° C. for 16 h. The reaction mixture was diluted with ethyl acetate, filtered through Celite and washed with ethyl acetate. The filtrate was washed with water and brine, dried over anhydrous sodium sulphate, filtered and concentrated. The residue was purified by silica gel chromatography (35% ethyl acetate-hexane) to afford 1-(2,6-dibenzyloxy-3-pyridyl)-4-hydroxy-3-methyl-benzimidazol-2-one (7, 831 mg, 1.83 mmol, 90% yield). LCMS: m/z 454.3 [M+H]⁺

### Step 6: tert-butyl 4-[1-(2,6-dibenzyloxy-3-pyridyl)-3-methyl-2-oxo-benzimidazol-4-yl]oxypiperidine-1-carboxylate (9)

To a stirred solution of 1-(2,6-dibenzyloxy-3-pyridyl)-4-hydroxy-3-methyl-benzimidazol-2-one (7, 0.8 g, 1.76 mmol) in DMF (8 mL) was added cesium carbonate (1.72 g, 5.29 mmol) and tert-butyl 4-((methylsulfonyl)oxy)piperidine-1-carboxylate (8, 511.04 mg, 1.83 mmol). The solution was heated at 90° C. for 16 h. The reaction mixture was diluted with ethyl acetate and washed with water and brine. The organic layer was dried over anhydrous sodium sulphate, filtered and concentrated under reduced pressure. The residue was purified by silica gel chromatography to afford tert-butyl 4-[1-(2,6-dibenzyloxy-3-pyridyl)-3-methyl-2-oxo-benzimidazol-4-yl]oxypiperidine-1-carboxylate (9, 0.56 g, 879.49 μmol, 50% yield). LCMS: m/z 637.5 [M+H]⁺

### Step 7: tert-butyl 4-[1-(2,6-dioxo-3-piperidyl)-3-methyl-2-oxo-benzimidazol-4-yl]oxypiperidine-1-carboxylate (10)

To a solution of tert-butyl 4-[1-(2,6-dibenzyloxy-3-pyridyl)-3-methyl-2-oxo-benzimidazol-4-yl]oxypiperidine-1-carboxylate (9, 3.5 g, 5.50 mmol) in Ethyl acetate (30 mL), Pd/C (10% by wt, 50% wet) (3.5 g, 5.50 mmol) was added. The resulting mixture was stirred under hydrogen balloon pressure for 16 h. The reaction mixture was filtered through Celite, washed with ethyl acetate and concentrated under reduced pressure. The residue was purified by silica gel chromatography (50% ethyl acetate in hexanes) to afford tert-butyl 4-[1-(2,6-dioxo-3-piperidyl)-3-methyl-2-oxo-benzimidazol-4-yl]oxypiperidine-1-carboxylate (10, 1.42 g, 3.10 mmol, 56% yield). LCMS: m/z 459.4 [M+H]⁺

### Step 8: 3-[3-methyl-2-oxo-4-(4-piperidyloxy)benzimidazol-1-yl]piperidine-2,6-dione (11)

HCl (4M in dioxane) (32.71 mmol, 7.5 mL) was added to tert-butyl 4-[1-(2,6-dioxo-3-piperidyl)-3-methyl-2-oxo-benzimidazol-4-yl]oxypiperidine-1-carboxylate (10, 1.5 g, 3.27 mmol) at 10° C. The mixture was warmed to RT and stirred for 16 h. The reaction mixture was concentrated under reduced pressure, triturated with ether and lyophilized to obtain 3-[3-methyl-2-oxo-4-(4-piperidyloxy)benzimidazol-1-yl]piperidine-2,6-dione (11, 1.25 g, 3.13 mmol, 96% yield, HCl salt) as an off white solid. LCMS: m/z 359.3 [M+H]⁺

¹H NMR (400 MHZ, DMSO-d6) δ 11.09 (s, 1H), 9.08 (bs, 1H), 8.85 (bs, 1H), 6.97 (t, J=8.0 Hz, 1H), 6.83 (d, J=8.3 Hz, 1H), 6.77 (d, J=7.7 Hz, 1H), 5.37-5.33 (m, 1H), 4.76 (s, 1H), 3.55 (s, 3H), 3.20 (s, 2H), 3.10 (s, 2H), 2.92-2.85 (m, 1H), 2.72-2.59 (m, 2H), 2.15-2.14 (m, 2H), 2.00-1.97 (m, 3H).

### 2-[4-[1-(2,6-dioxo-3-piperidyl)-2-oxo-benzo[cd]indol-6-yl]-1-piperidyl]acetic acid (11)

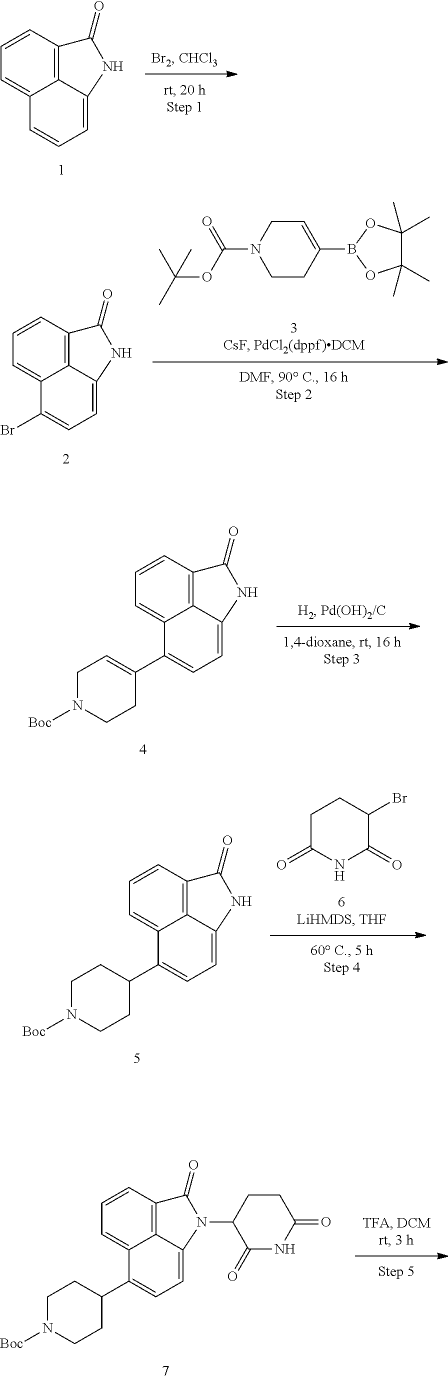

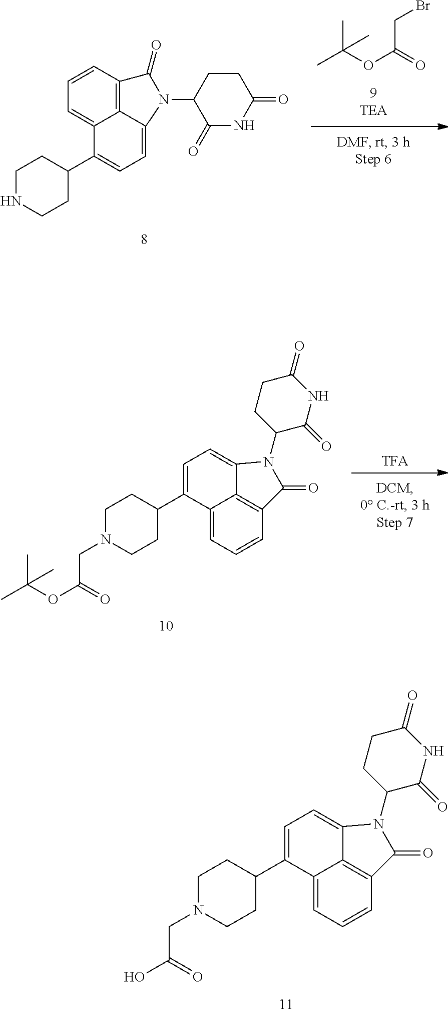

### Step 1: 6-bromo-1H-benzo[cd]indol-2-one (2)

Into a 1 L two neck round bottom flask containing a well-stirred solution of 1H-benzo[cd]indol-2-one (1, 5 g, 29.55 mmol, 60.24 μL) in CHCl₃ (300 mL) was added bromine (3.59 g, 44.33 mmol, 2.41 mL) at 0° C. The reaction mixture was stirred at RT for 20 h. The reaction mixture was quenched with aqueous sodium thiosulphate solution (200 mL) at 0° C. The yellow precipitate was filtered and washed with cold water (250 mL) and diethyl ether (150 mL) to afford 6-bromo-1H-benzo[cd]indol-2-one (2, 5.8 g, 21.51 mmol, 73% yield) as a yellow solid. LCMS (ES+): m/z 250.1 [M+H]⁺

### Step 2: tert-butyl 4-(2-oxo-1H-benzo[cd]indol-6-yl)-3,6-dihydro-2H-pyridine-1-carboxylate (4)

Into a 250 mL pressure tube containing a well-stirred suspension of 6-bromo-1H-benzo[cd]indol-2-one (2, 4 g, 16.12 mmol, 60.24 μL) in DMF (30 mL) was added tert-butyl 4-(4,4,5,5-tetramethyl-1,3,2-dioxaborolan-2-yl)-3,6-dihydro-2H-pyridine-1-carboxylate (3, 5.48 g, 17.74 mmol) and cesium fluoride (4.90 g, 32.25 mmol, 1.19 mL). The mixture was purged with nitrogen for 5 min. Then [1,1′-bis(diphenylphosphino)ferrocene]dichloropalladium(II) dichloromethane complex (1.5 g, 2.42 mmol) was added. The reaction mixture was heated at 90° C. for 16 h. The reaction mixture was concentrated under reduced pressure. The residue was purified by silica gel chromatography (50-60% EtOAc in petroleum ether) to afford tert-butyl 4-(2-oxo-1H-benzo[cd]indol-6-yl)-3,6-dihydro-2H-pyridine-1-carboxylate (4, 2.1 g, 3.23 mmol, 31% yield) as a pale yellow solid. LCMS (ES+): m/z 351.2 [M+H]⁺

### Step 3: tert-butyl 4-(2-oxo-1H-benzo[cd]indol-6-yl)piperidine-1-carboxylate (5)

Into a 250 mL single neck round bottom flask containing a well-stirred solution of tert-butyl 4-(2-oxo-1H-benzo[cd]indol-6-yl)-3,6-dihydro-2H-pyridine-1-carboxylate (4, 2.2 g, 6.28 mmol) in 1,4-dioxane (80) mL) was added palladium hydroxide (20% on carbon) (1.5 g, 6.28 mmol) and the mixture stirred under a hydrogen balloon atmosphere for 16 h at RT. The reaction mixture was filtered through Celite and washed with EtOAc (300 mL). The solvent was removed under reduced pressure to obtain tert-butyl 4-(2-oxo-1H-benzo[cd]indol-6-yl)piperidine-1-carboxylate (5, 2 g. 4.90 mmol, 78% yield) as a pale yellow solid. LCMS (ES+): m/z 353.1 [M+H]⁺

### Step 4: tert-butyl 4-[1-(2,6-dioxo-3-piperidyl)-2-oxo-benzo[cd]indol-6-yl]piperidine-1-carboxylate (7)

Into a 250 mL two neck round bottom flask containing a well-stirred solution of tert-butyl 4-(2-oxo-1H-benzo[cd]indol-6-yl)piperidine-1-carboxylate (5, 2 g, 5.67 mmol) in THF (20 mL) was added lithium bis(trimethylsilyl)amide (1.0 M in THF) (12.2 mmol, 12 mL) at 0° C. After 30 min at (° C.), 3-bromopiperidine-2,6-dione (6, 1.09 g, 5.67 mmol) was added in portions. The reaction mixture was stirred at 60° C. for 5 h. The reaction mixture was cooled to (° C.) and 1.5 N HCl (4 mL) added to adjust the pH to 3-4. The mixture was diluted with EtOAc (400 mL) and layers partitioned. The organic layer was washed with water (200 mL) and brine (150 mL). The combined organic layer was dried over anhydrous Na₂SO₄, filtered, concentrated and purified by reverse phase prep HPLC (Column: YMC C-18 (150×30 mm), 5 μm, Mobile phase A: 0.1% TFA in water; Mobile phase B: MeCN) to obtain tert-butyl 4-[1-(2,6-dioxo-3-piperidyl)-2-oxo-benzo[cd]indol-6-yl]piperidine-1-carboxylate (7, 1 g, 1.69 mmol, 30% yield) as a pale yellow solid. LCMS (ES−): m/z 462.2 [M−H]⁻

### Step 5: 3-[2-oxo-6-(4-piperidyl)benzo[cd]indol-1-yl]piperidine-2,6-dione (8)

Into a 50 mL single neck round bottom flask containing a well-stirred solution of tert-butyl 4-[1-(2,6-dioxo-3-piperidyl)-2-oxo-benzo[cd]indol-6-yl]piperidine-1-carboxylate (7, 100 mg, 215.74 μmol) in DCM (1 mL) was added trifluoroacetic acid (740.00 mg, 6.49 mmol, 0.5 mL) at 0° C. The reaction mixture was stirred at RT for 3 h. The reaction mixture was concentrated under reduced pressure. The residue was triturated with diethyl ether (0.7 mL) and filtered to obtain 3-[2-oxo-6-(4-piperidyl)benzo[cd]indol-1-yl]piperidine-2,6-dione (8, 80 mg, 163.21 μmol, 76% yield) as a pale yellow solid. LCMS (ES+): m/z 364.1 [M+H]⁺

### Step 6: Preparation of tert-butyl 2-[4-[1-(2,6-dioxo-3-piperidyl)-2-oxo-benzo[cd]indol-6-yl]-1-piperidyl]acetate (10)

Into a 10 mL single neck round bottom flask containing a well-stirred solution of 3-[2-oxo-6-(4-piperidyl)benzo[cd]indol-1-yl]piperidine-2,6-dione (8, 65 mg, 136.15 μmol) in DMF (1 mL) was added triethylamine (68.88 mg, 680.73 μmol, 94.88 μL) at RT. Tert-butyl 2-bromoacetate (9, 31.87 mg, 163.37 μmol, 23.96 μL) was then added at 0° C.) The reaction mixture was stirred for 3 h at RT. The reaction mixture was quenched and stirred with cold water (0.5 mL), and pale yellow precipitate was collected by filtration, washed with cold water (0.5 mL) and diethyl ether (1 mL) to afford tert-butyl 2-[4-[1-(2,6-dioxo-3-piperidyl)-2-oxo-benzo[cd]indol-6-yl]-1-piperidyl]acetate (10, 64 mg, 132.14 μmol, 97% yield) as a pale yellow solid. LCMS (ES+): m/z 478.1 [M+H]⁺

### Step 7: Preparation of 2-[4-[1-(2,6-dioxo-3-piperidyl)-2-oxo-benzo[cd]indol-6-yl]-1-piperidyl]acetic acid (11)

Into a 10 mL single neck round bottom flask containing a well-stirred solution of tert-butyl 2-[4-[1-(2,6-dioxo-3-piperidyl)-2-oxo-benzo[cd]indol-6-yl]-1-piperidyl]acetate (10, 70 mg, 146.58 μmol) in DCM (1 mL) was added trifluoroacetic acid (382.23 mg, 3.35 mmol, 258.26 μL) at 0° C. The reaction mixture was stirred at RT for 3 h. The reaction mixture was concentrated under reduced pressure. The resulting residue was washed with diethyl ether (0.6 mL) to obtain 2-[4-[1-(2,6-dioxo-3-piperidyl)-2-oxo-benzo[cd]indol-6-yl]-1-piperidyl]acetic acid (11, 71 mg, 128.75 μmol, 88% yield) as an off-white solid. LCMS (ES+): m/z 422.1 [M+H]⁺

### 2-[4-[4-[(2,6-dioxo-3-piperidyl)amino]-2-fluoro-phenyl]-1-piperidyl]acetic acid (9)

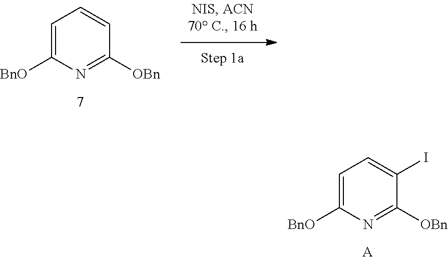

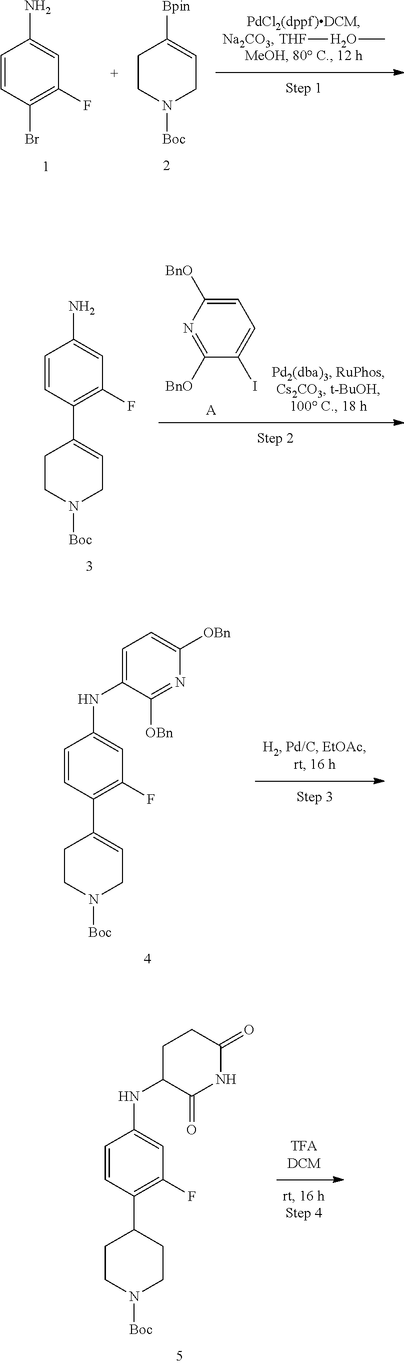

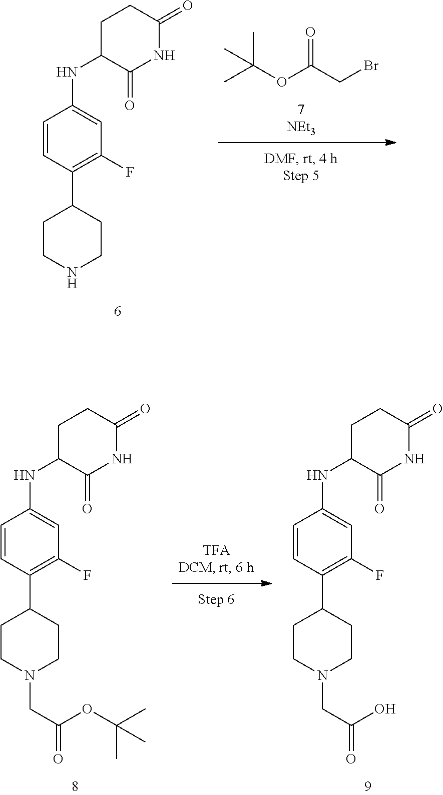

### Step 1a: 2,6-dibenzyloxy-3-iodo-pyridine (A)

To a stirred solution of 2,6-dibenzyloxypyridine (7, 50 g, 171.62 mmol) in acetonitrile (900 mL) was added N-Iodosuccinimide (38.61 g, 171.62 mmol) portion-wise and stirred for 10 min at rt before heating at 80° C. After 16 h, the solvent was removed under reduced pressure. The residue was diluted with ethyl acetate, washed with saturated sodium thiosulphate (2×50 mL), water (2×20 mL) and brine (1×10 mL). The combined organic layer was dried over sodium sulphate, filtered and concentrated to obtain 2,6-dibenzyloxy-3-iodo-pyridine (A, 60 g, 115.04 mmol, 67% yield). LCMS (ES+): m/z 418.2 [M+H]⁺

### Step 1: 4-(4-Amino-2-fluoro-phenyl)-3, 6-dihydro-2H-pyridine-1-carboxylic acid tert-butyl ester (3)

To a stirred solution of 4-bromo-3-fluoro-aniline (1, 5.00 g, 26.31 mmol) and tert-butyl 4-(4,4,5,5-tetramethyl-1,3,2-dioxaborolan-2-yl)-3,6-dihydro-2H-pyridine-1-carboxylate (2, 8.95 g, 28.95 mmol) in water (12 mL), THF (60 mL) and methanol (24 mL) was added sodium carbonate (6.14 g, 57.89 mmol) and the mixture purged with argon. PdCl₂(dppf) dichloromethane complex (429.78 mg, 526.28 μmol) was added and purged again. The reaction mixture was heated at 80° C. for 12 h. The reaction mixture was diluted with ethyl acetate, filtered through a pad of Celite and washed with ethyl acetate. The filtrate was washed with water and brine, dried over anhydrous sodium sulphate, filtered and concentrated. The residue was purified by silica gel chromatography (15% ethyl acetate-hexane) to obtain tert-butyl 4-(4-amino-2-fluoro-phenyl)-3,6-dihydro-2H-pyridine-1-carboxylate (3, 6.1 g, 20.87 mmol, 79% yield) as pale yellow solid. LCMS (ES+): m/z 293 [M+H]⁺

### Step 2: 4-[4-(2,6-Bis-benzyloxy-pyridin-3-ylamino)-2-fluoro-phenyl]-3,6-dihydro-2H-pyridine-1-carboxylic acid tert-butyl ester (4)

Cesium carbonate (19.73 g, 60.54 mmol) was added to a stirred solution of tert-butyl 4-(4-amino-2-fluoro-phenyl)-3,6-dihydro-2H-pyridine-1-carboxylate (3, 5.9 g, 20.18 mmol) and 2,6-dibenzyloxy-3-iodo-pyridine (A, 9.26 g, 22.20 mmol) in t-BuOH (60 mL). The resulting mixture was purged with argon and Pd₂(dba)₃ (924.02 mg, 1.01 mmol) and RuPhos (941.73 mg, 2.02 mmol) were added under inert atmosphere. The mixture was heated at 100° C. for 18 h. The reaction mixture was diluted with ethyl acetate, filtered through Celite and washed with ethyl acetate. The combined organic layer was washed with water and brine, dried over anhydrous sodium sulphate, filtered and concentrated under reduced pressure. The residue was purified by column chromatography (15% ethyl acetate-hexane) to obtain tert-butyl 4-[4-[(2,6-dibenzyloxy-3-pyridyl)amino]-2-fluoro-phenyl]-3,6-dihydro-2H-pyridine-1-carboxylate (4, 5.9 g, 10.14 mmol, 50% yield) as pale yellow solid. LCMS (ES+): m/z 582 [M+H]⁺

### Step 3: 4-[4-(2,6-Dioxo-piperidin-3-ylamino)-2-fluoro-phenyl]-piperidine-1-carboxylic acid tert-butyl ester (5)

To a stirred solution of tert-butyl 4-[4-[(2,6-dibenzyloxy-3-pyridyl)amino]-2-fluoro-phenyl]-3,6-dihydro-2H-pyridine-1-carboxylate (4, 4.6 g, 7.91 mmol) in ethyl acetate (40 mL) was added 10% Pd—C(50% wet, 4.6 g). The resulting mixture was stirred at RT under hydrogen atmosphere for 20 h. The reaction mixture was filtered through Celite and washed with ethyl acetate. The solvent was removed under reduced pressure and the residue purified by silica gel chromatography (40% ethyl acetate-hexane) to afford tert-butyl 4-[4-[(2,6-dioxo-3-piperidyl)amino]-2-fluoro-phenyl]piperidine-1-carboxylate (5, 2.6 g, 6.41 mmol, 81% yield) as blue solid. LCMS (ES+): m/z 406 [M+H]⁺

### Step 4: 3-(3-Fluoro-4-piperidin-4-yl-phenylamino)-piperidine-2, 6-dione (6)

To a stirred solution of tert-butyl 4-[4-[(2,6-dioxo-3-piperidyl)amino]-2-fluoro-phenyl]piperidine-1-carboxylate (1 g, 2.47 mmol) in DCM (6 mL) was added 2,2,2-trifluoroacetic acid (1.69 g, 14.80 mmol, 1.14 mL) at (° C.) and then stirred at RT for 1.5 h. The reaction mixture was concentrated under reduced pressure and diluted by ether and stirred for 1 h. Ether was decanted to afford 3-[3-fluoro-4-(4-piperidyl)anilino]piperidine-2,6-dione (0.87 g, 1.87 mmol, 75.70% yield) as an off white solid. LCMS (ES+): m/z 306 [M+H]⁺

### Step 5: tert-butyl 2-[4-[4-[(2,6-dioxo-3-piperidyl)amino]-2-fluoro-phenyl]-1-piperidyl]acetate (8)

Into a 250 mL round bottom flask containing a well-stirred solution of 3-[3-fluoro-4-(4-piperidyl)anilino]piperidine-2,6-dione (6, 4 g, 13.10 mmol) and tert-butyl 2-bromoacetate (7, 2.81 g, 14.41 mmol, 2.11 mL) in anhydrous DMF (50 mL) was added TEA (3.98 g, 39.30 mmol, 5.48 mL). The reaction mixture was stirred at RT for 4 h. The reaction mixture was diluted with water and extracted with EtOAc (200 mL). The organic layer was dried over anhydrous Na₂SO₄, filtered and concentrated under reduced pressure. The residue was purified by silica gel chromatography (0-100% ethyl acetate in petroleum ether) to afford tert-butyl 2-[4-[4-[(2,6-dioxo-3-piperidyl)amino]-2-fluoro-phenyl]-1-piperidyl]acetate (8, 4 g, 9.43 mmol, 72% yield). LCMS (ESI): m/z 420.3 [M+H]⁺

### Step 6: 2-[4-[4-[(2,6-dioxo-3-piperidyl)amino]-2-fluoro-phenyl]-1-piperidyl]acetic acid (9)

To a stirred solution of tert-butyl 2-[4-[4-[(2,6-dioxo-3-piperidyl)amino]-2-fluoro-phenyl]-1-piperidyl]acetate (8, 2.4 g, 5.72 mmol) in DCM (30 mL) was added TFA (5.22 g, 45.77 mmol, 3.53 mL) at 0° C. The reaction was stirred at RT for 8 h. The solvent was removed, and the residue triturated with diethyl ether. The final compound was dried under reduced pressure to afford 2-[4-[4-[(2,6-dioxo-3-piperidyl)amino]-2-fluoro-phenyl]-1-piperidyl]acetic acid (9, 2.7 g, 5.22 mmol, 91% yield, TFA salt) as a bluish-green solid. LCMS (ES+): m/z 364.0 [M+H]⁺

### Preparation of Exemplary Formula (I) Compounds

### Synthesis of N-(2-((7-(1,1-dioxido-4-oxo-1,2,5-thiadiazolidin-2-yl)-8-fluoro-6-hydroxynaphthalen-2-yl)oxy)ethyl)-1-((2-(2,6-dioxopiperidin-3-yl)-1,3-dioxoisoindolin-4-yl)glycyl)piperidine-4-carboxamide (Example 1)

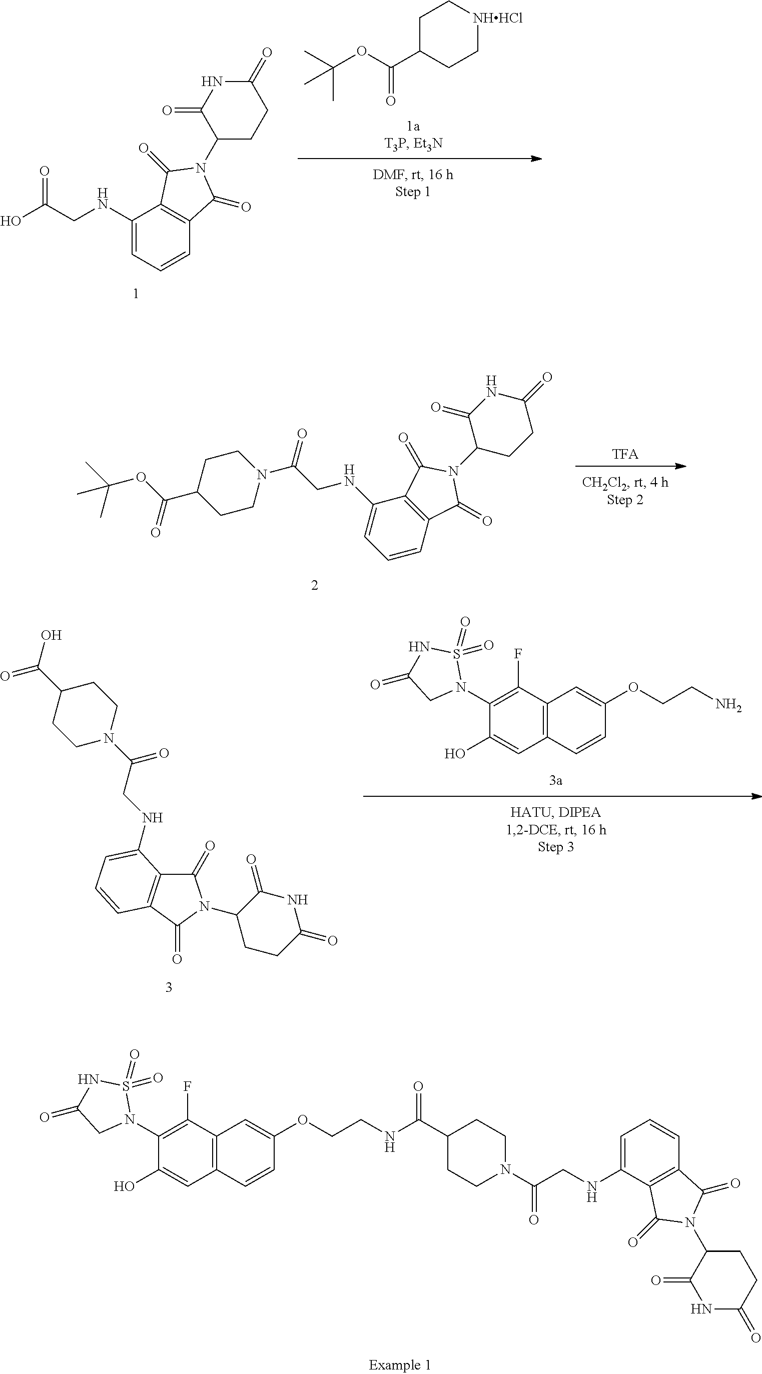

### Step 1: tert-butyl 1-((2-(2,6-dioxopiperidin-3-yl)-1,3-dioxoisoindolin-4-yl)glycyl)piperidine-4-carboxylate (2)

Into a 25 mL single neck round bottom flask containing a well-stirred solution of tert-butyl piperidine-4-carboxylate hydrochloride (1a, 93.34 mg, 421.06 μmol) in DMF (2.5 mL) at RT under nitrogen atmosphere were added propylphosphonic anhydride solution (50 wt. % in EtOAc) (107.18 mg, 336.85 μmol, 214 μL) and Et₃N (102.26 mg, 1.01 mmol, 140.85 μL). The resulting mixture was stirred at RT for 5 min. Then, 2-[[2-(2,6-dioxo-3-piperidyl)-1,3-dioxo-isoindolin-4-yl]amino]acetic acid (1, 150 mg, 336.85 μmol, TFA salt) was added and the mixture was stirred at RT for 16 h. The reaction mixture was concentrated under reduced pressure and the residue was triturated with Et₂O (2×20 mL) to afford tert-butyl 1-((2-(2,6-dioxopiperidin-3-yl)-1,3-dioxoisoindolin-4-yl)glycyl)piperidine-4-carboxylate (2, 180 mg, 312.68 μmol, 93% yield) as an off-white solid.

LCMS (ES+): m/z 499.0 [M+H]⁺

### Step 2: 1-((2-(2,6-dioxopiperidin-3-yl)-1,3-dioxoisoindolin-4-yl)glycyl)piperidine-4-carboxylic acid (3)

Into a 25 mL single neck round bottom flask containing a well-stirred solution of tert-butyl 1-[2-[[2-(2,6-dioxo-3-piperidyl)-1,3-dioxo-isoindolin-4-yl]amino]acetyl]piperidine-4-carboxylate (2, 180 mg, 361.06 μmol) in DCM (3 mL) at RT under nitrogen atmosphere was added TFA (1.48 g, 12.98 mmol, 1.0 mL) and the resulting mixture was stirred for 2 h. The reaction mixture was concentrated under reduced pressure, azeotroped with toluene (2×10 mL), triturated with Et₂O (2×20 mL), filtered and dried to afford 1-((2-(2,6-dioxopiperidin-3-yl)-1,3-dioxoisoindolin-4-yl)glycyl)piperidine-4-carboxylic acid (3, 150 mg, 233.45 μmol, 65% yield, TFA salt) as an off-white solid which was used without further purification. LCMS (ES+): m/z 443.0 [M+H]⁺

### Step 3: N-(2-((7-(1,1-dioxido-4-oxo-1,2,5-thiadiazolidin-2-yl)-8-fluoro-6-hydroxy naphthalen-2-yl)oxy)ethyl)-1-((2-(2,6-dioxopiperidin-3-yl)-1,3-dioxoisoindolin-4-yl)glycyl) piperidine-4-carboxamide (Example 1)

Into a 25 mL single neck round bottom flask containing a well-stirred solution of 5-[7-(2-aminoethoxy)-1-fluoro-3-hydroxy-2-naphthyl]-1,1-dioxo-1,2,5-thiadiazolidin-3-one hydrochloride (3a, 35.21 mg, 89.86 μmol) in DMF (0.2 mL) at RT under nitrogen atmosphere were added DIPEA (34.84 mg, 269.57 μmol, 46.95 μL) and HATU (68.33 mg, 179.71 μmol). The resulting mixture was stirred at RT for 4 h. Then, 1-[2-[[2-(2,6-dioxo-3-piperidyl)-1,3-dioxo-isoindolin-4-yl]amino]acetyl]piperidine-4-carboxylic acid (3, 50 mg, 89.86 μmol, TFA salt) in 1,2-DCE (0.2 mL) was added and the reaction mixture was stirred at RT for 12 h. The reaction mixture was concentrated under reduced pressure, and the residue purified by reverse-phase preparative HPLC [Column: X-BRIDGE C18 (19×150 mm) 5.0 μm with Mobile phase A: 0.1% TFA in water; Mobile phase B: Acetonitrile] to afford N-(2-((7-(1,1-dioxido-4-oxo-1,2,5-thiadiazolidin-2-yl)-8-fluoro-6-hydroxynaphthalen-2-yl)oxy)ethyl)-1-((2-(2,6-dioxopiperidin-3-yl)-1,3-dioxoisoindolin-4-yl)glycyl)piperidine-4-carboxamide (Example 1, 8 mg, 8.60 μmol, 10% yield, TFA salt) as a yellow solid.

LCMS (ES+): m/z 780.1 [M+H]⁺

¹H NMR (400 MHZ, DMSO-d₆) δ 11.09 (s, 1H), 9.52 (s, 1H), 8.11 (dd, J=5.6 Hz, 1H), 7.67 (d, J=9.0 Hz, 1H), 7.60 (t, J=7.8 Hz, 1H), 7.22-7.18 (m, 1H), 7.17-7.02 (m, 5H), 5.06 (dd, J=12.8, 5.4 Hz, 1H), 4.35 (d, J=13.1 Hz, 1H), 4.25-4.15 (m, 3H), 4.13-4.06 (m, 4H), 3.89 (d, J=13.3 Hz, 1H), 2.95-2.82 (m, 4H), 2.75-2.62 (m, 3H), 2.09-2.00 (m, 1H), 1.80-1.70 (m, 2H), 1.64-1.53 (m, 1H), 1.52-1.36 (m, 1H).

### Synthesis of N-(2-((7-(1,1-dioxido-4-oxo-1,2,5-thiadiazolidin-2-yl)-8-fluoro-6-hydroxynaphthalen-2-yl)oxy)ethyl)-1-((2-(2,6-dioxopiperidin-3-yl)-1-oxoisoindolin-4-yl)glycyl)piperidine-4-carboxamide (Example 2)

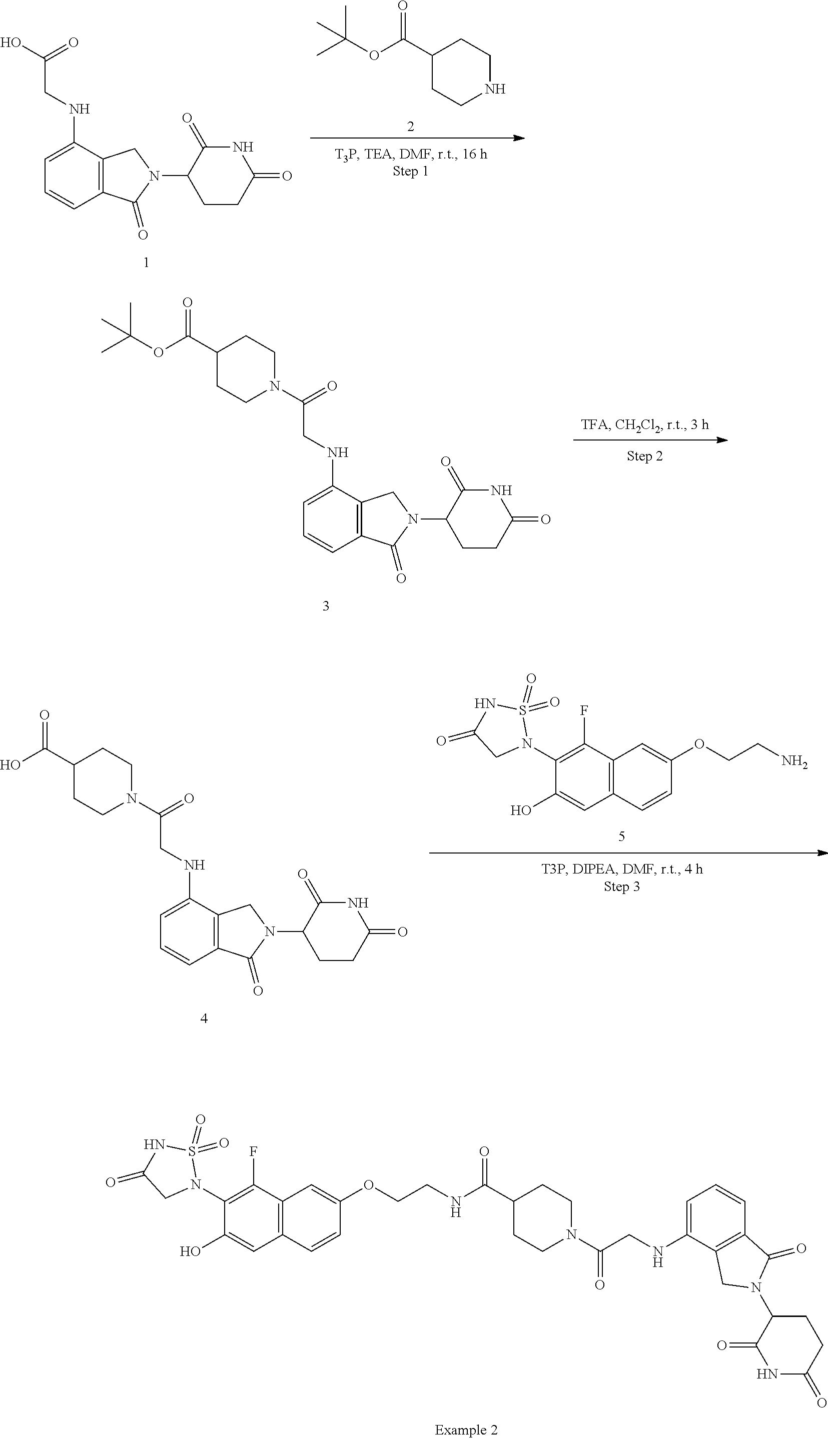

### Step 1: tert-butyl 1-((2-(2,6-dioxopiperidin-3-yl)-1-oxoisoindolin-4-yl)glycyl)piperidine-4-carboxylate (3)

Into a 50 mL single neck round bottom flask containing a well-stirred solution of 2-[[2-(2,6-dioxo-3-piperidyl)-1-oxo-isoindolin-4-yl]amino]acetic acid (1, 516.16 mg, 1.63 mmol), tert-butyl piperidine-4-carboxy late hydrochloride (2, 0.35 g, 1.36 mmol) in anhydrous DMF (8 mL) were added propylphosphonic anhydride solution (50 wt. % in ethyl acetate) (1.72 mL, 862.66 mg, 2.71 mmol) and TEA (548.70 mg, 5.42 mmol, 755.79 μL). After 16 h, the reaction mixture was poured over ice cold water and extracted with EtOAc (2×30 mL). The combined organic layer was dried over anhydrous Na₂SO₄ and filtered. The solvent was removed under reduced pressure and the residue purified by silica gel chromatography (100% EtOAc) to obtain tert-butyl 1-[2-[[2-(2,6-dioxo-3-piperidyl)-1-oxo-isoindolin-4-yl]amino]acetyl]piperidine-4-carboxylate (3, 0.35 g, 571 μmol, 33% yield) as an off-white solid. LCMS (ES−): m/z 483.3 [M−H]⁻

### Step 2: 1-((2-(2,6-dioxopiperidin-3-yl)-1-oxoisoindolin-4-yl)glycyl)piperidine-4-carboxylic acid (4)

Into a 100 mL single neck round bottom flask containing a well-stirred solution of tert-butyl 1-[2-[[2-(2,6-dioxo-3-piperidyl)-1-oxo-isoindolin-4-yl]amino]acetyl]piperidine-4-carboxylate (3, 0.35 g, 571 μmol) in DCM (10 mL) was added TFA (1.49 g, 13.06 mmol, 1 mL) at RT. The resulting solution was stirred at RT for 3 h. The volatiles were removed from the reaction mixture and the residue washed with petroleum ether (30 mL) to obtain 1-[2-[[2-(2,6-dioxo-3-piperidyl)-1-oxo-isoindolin-4-yl]amino]acetyl]piperidine-4-carboxylic acid (4, 0.2 g, 260.59 μmol, 45% yield, TFA salt) as an off-white solid. LCMS (ES−): m/z 427.2 [M−H]⁻

### Step 3: N-(2-((7-(1,1-dioxido-4-oxo-1,2,5-thiadiazolidin-2-yl)-8-fluoro-6-ethyl)-1-((2-(2,6-dioxopiperidin-3-yl)-1-oxoisoindolin-4-hydroxynaphthalen-2-yl)oxy) yl)glycyl)piperidine-4-carboxamide (Example 2)

Into a 10 mL single neck round bottom flask containing well-stirred solution of 5-[7-(2-aminoethoxy)-1-fluoro-3-hydroxy-2-naphthyl]-1,1-dioxo-1,2,5-thiadiazolidin-3-one (5, 40 mg, 102.09 μmol, HCl salt) and 1-[2-[[2-(2,6-dioxo-3-piperidyl)-1-oxo-isoindolin-4-yl]amino]acetyl]piperidine-4-carboxylic acid (4, 43.74 mg, 80.63 μmol, TFA salt) in anhydrous DMF (0.5 mL) were added DIPEA (65.97 mg, 510.46 μmol, 88.91 μL) and propylphosphonic anhydride (50 wt. % in ethyl acetate) (0.13 mL, 64.97 mg, 204.18 μmol) at RT. The reaction mixture was stirred at RT for 4 h. The reaction was quenched with water, separated and the organic layer concentrated under reduced pressure and purified by reverse phase prep HPLC [Purification method: Column: Sunfire C18 (19×150 mm) 5 μm, mobile phase: 0.1% TFA in water and MeCN] to afford 1-[2-[[2-(2,6-dioxo-3-piperidyl)-1-oxo-isoindolin-4-yl]amino]acetyl]-N-[2-[[8-fluoro-6-hydroxy-7-(1,1,4-trioxo-1,2,5-thiadiazolidin-2-yl)-2-naphthyl]oxy]ethyl]piperidine-4-carboxamide (Example 2, 22 mg, 23.12 μmol, 23% yield, TFA salt) as a colorless solid.

LCMS (ES+): m/z 766.4 [M+H]⁺

¹H NMR (400 MHZ, DMSO-d₆) δ 10.99 (s, 1H), 10.24 (s, 1H), 8.12 (t, J=5.6 Hz, 1H), 7.72 (d, J=9.0 Hz, 1H), 7.29 (t, J=7.7 Hz, 1H), 7.24-7.22 (m, 1H), 7.21-7.17 (m, 1H), 7.07 (s, 1H), 6.98 (d, J=7.4 Hz, 1H), 6.77 (d, J=8.1 Hz, 1H), 5.11 (dd, J=13.3, 5.1 Hz, 1H), 4.44 (s, 2H), 4.38-4.27 (m, 2H), 4.17 (d, J=17.0 Hz, 1H), 4.13-4.08 (m, 2H), 4.06-4.03 (m, 2H), 3.99-3.92 (m, 1H), 3.52-3.45 (m, 3H), 3.05 (t, J=12.7 Hz, 1H), 2.99-2.85 (m, 1H), 2.70-2.57 (m, 2H), 2.44-2.31 (m, 2H), 2.06-1.99 (m, 1H), 1.78-1.68 (m, 2H), 1.63-1.35 (m, 3H), 1.01-0.91 (m, 1H).

### 1-[2-[2-(2,6-dioxo-3-piperidyl)-1-oxo-isoindolin-4-yl]oxyacetyl]-N-[2-[[8-fluoro-6-hydroxy-7-(1,1,4-trioxo-1,2,5-thiadiazolidin-2-yl)-2-naphthyl]oxy]ethyl]piperidine-4-carboxamide

### Example 3

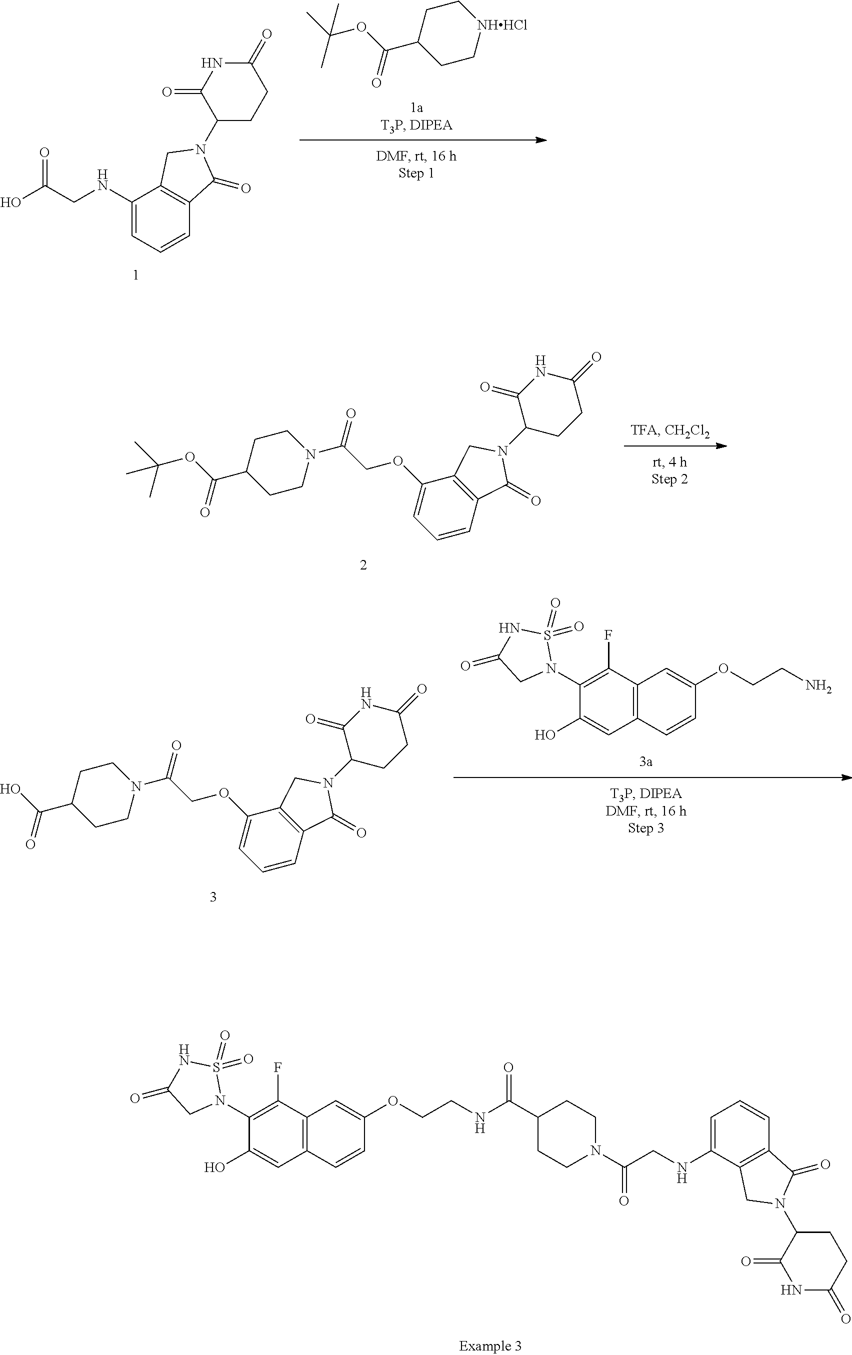

### Step 1 tert-butyl 1-(2-((2-(2,6-dioxopiperidin-3-yl)-1-oxoisoindolin-4-yl)oxy)acetyl) piperidine-4-carboxylate (2)

Into a 25 mL single neck round bottom flask containing a well-stirred solution of 2-[2-(2,6-dioxo-3-piperidyl)-1-oxo-isoindolin-4-yl]oxyacetic acid (1, 200 mg, 628.38 μmol) in DMF (2.5 mL) were added propylphosphonic anhydride (50 wt. % in EtOAc) (199.94 mg, 628.38 μmol, 400 μL) and Et₃N (190.76 mg, 1.89 mmol, 262.75 μL). The resulting mixture was stirred at RT for 15 min. Then, tert-butyl piperidine-4-carboxylate hydrochloride (1a, 167.19 mg, 754.05 μmol) was added and the reaction mixture was stirred at RT for 16 h. The reaction was concentrated under reduced pressure and purified by silica gel chromatography (0-20% MeOH/DCM) to afford tert-butyl 1-(2-((2-(2,6-dioxopiperidin-3-yl)-1-oxoisoindolin-4-yl)oxy)acetyl)piperidine-4-carboxylate (2, 210 mg, 415.22 μmol, 66% yield) as an off-white solid. LCMS (ES+): m/z 430.1 [M-t-Bu+H]⁺

### Step 2: 1-(2-((2-(2,6-dioxopiperidin-3-yl)-1-oxoisoindolin-4-yl)oxy)acetyl)piperidine-4-carboxylic acid (3)

Into a 25 mL single neck round bottom flask containing a well-stirred solution of tert-butyl 1-[2-[2-(2,6-dioxo-3-piperidyl)-1-oxo-isoindolin-4-yl]oxyacetyl]piperidine-4-carboxylate (2, 200 mg, 411.92 μmol) in DCM (3 mL) was added TFA (469.67 mg, 4.12 mmol, 317.35 μL). After 2 h, the reaction mixture was concentrated under reduced pressure, and the residue azeotroped with toluene (2×10 mL) and triturated with diethyl ether (2×20 mL) to afford 1-(2-((2-(2,6-dioxopiperidin-3-yl)-1-oxoisoindolin-4-yl)oxy)acetyl)piperidine-4-carboxylic acid (3, 170 mg, 367.14 μmol, 89% yield) as an off-white solid which was used without further purification. LCMS (ES+): m/z 430.0 [M+H]⁺

### Step 3: 1-[2-[2-(2,6-dioxo-3-piperidyl)-1-oxo-isoindolin-4-yl]oxyacetyl]-N-[2-[[8-fluoro-6-hydroxy-7-(1,1,4-trioxo-1,2,5-thiadiazolidin-2-yl)-2-naphthyl]oxy]ethyl]piperidine-4-carboxamide (Example 3)

Into a 10 mL single neck round bottom flask containing a well-stirred solution of 1-[2-[2-(2,6-dioxo-3-piperidyl)-1-oxo-isoindolin-4-yl]oxyacetyl]piperidine-4-carboxylic acid (3, 40 mg, 0.0931 mmol) in DMF (0.5 mL) were added 5-[7-(2-aminoethoxy)-1-fluoro-3-hydroxy-2-naphthyl]-1,1-dioxo-1,2,5-thiadiazolidin-3-one hydrochloride (3a, 40.14 mg, 0.102 mmol), DIPEA (36.09 mg, 0.279 mmol, 48.64 μL) and propylphosphonic anhydride (50 wt. % in EtOAc) (37.02 mg, 0.116 mmol, 74 μL). After 16 h, the reaction was diluted with cold water (1 mL), concentrated under reduced pressure and purified by reverse-phase preparative HPLC [Column: X-BRIDGE C18 (19×150 mm) 5.0 μm with Mobile phase A: 0.1% TFA in water; Mobile phase B: Acetonitrile] to afford 1-[2-[2-(2,6-dioxo-3-piperidyl)-1-oxo-isoindolin-4-yl]oxyacetyl]-N-[2-[[8-fluoro-6-hydroxy-7-(1,1,4-trioxo-1,2,5-thiadiazolidin-2-yl)-2-naphthyl]oxy]ethyl]piperidine-4-carboxamide (Example 3, 20 mg, 21.42 μmol, 29% yield, TFA salt) as an off-white solid.

LCMS (ES+): m/z 767.4 [M+H]⁺

¹H NMR (400 MHZ, DMSO-d₆) δ 10.99 (s, 1H), 10.18 (s, 1H), 8.12 (t, J=5.6 Hz, 1H), 7.74-7.68 (m, 1H), 7.45 (t, J=7.8 Hz, 1H), 7.31 (d, J=7.4 Hz, 1H), 7.23 (d, J=2.6 Hz, 1H), 7.18 (dd, J=9.0, 2.5 Hz, 1H), 7.13 (d, J=8.2 Hz, 1H), 7.06 (s, 1H), 5.11 (dd, J=13.3, 5.1 Hz, 1H), 5.01 (d, J=3.8 Hz, 2H), 4.43-4.36 (m, 3H), 4.31-4.23 (m, 2H), 4.10 (t, J=5.7 Hz, 2H), 3.52-3.44 (m, 3H), 3.05 (t, J=12.7 Hz, 1H), 2.91 (ddd, J=17.9, 13.4, 5.3 Hz, 1H), 2.65-2.57 (m, 2H), 2.46-2.39 (m, 2H), 2.04-1.95 (m, 1H), 1.78-1.67 (m, 2H), 1.66-1.35 (m, 3H).

### N-(2-((7-(1,1-dioxido-4-oxo-1,2,5-thiadiazolidin-2-yl)-8-fluoro-6-hydroxynaphthalen-2-yl)oxy)ethyl)-2-(4-(1-(2,6-dioxopiperidin-3-yl)-3-methyl-2-oxo-2,3-dihydro-1H-benzo[d]imidazol-5-yl)piperidin-1-yl)acetamide (Example 4)

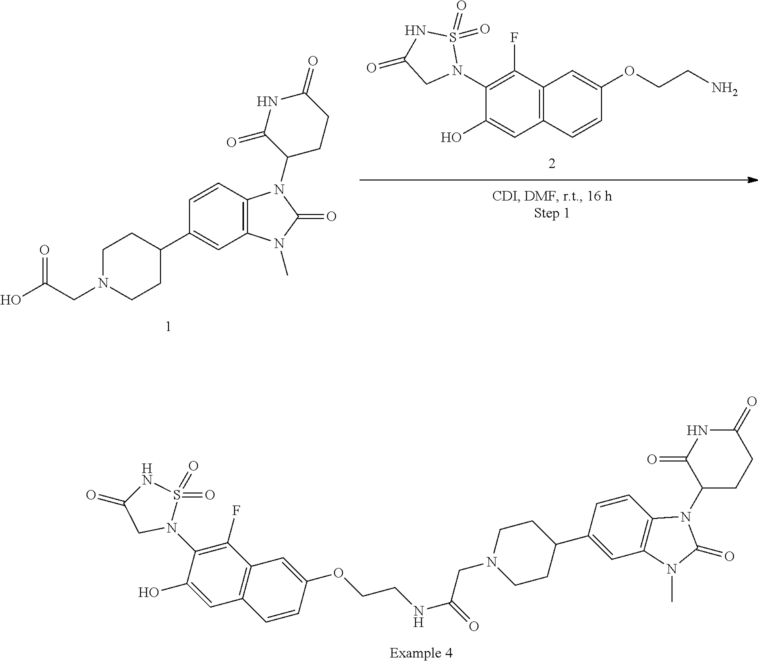 [SMILES: Cn1c(=O)n(C2CCC(=O)NC2=O)c2ccc(C3CCN(CC(=O)O)CC3)cc21 ; NCCOc1ccc2cc(O)c(N3CC(=O)NS3(=O)=O)c(F)c2c1 ; Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21]

### Step 1: N-(2-((7-(1,1-dioxido-4-oxo-1,2,5-thiadiazolidin-2-yl)-8-fluoro-6-hydroxy naphthalen-2-yl)oxy)ethyl)-2-(4-(1-(2,6-dioxopiperidin-3-yl)-3-methyl-2-oxo-2,3-dihydro-1H-benzo[d]imidazol-5-yl)piperidin-1-yl)acetamide (Example 4)

1,1′-Carbonyldiimidazole (145.84 mg, 899.43 μmol) was added to a well-stirred solution of 2-[4-[1-(2,6-dioxo-3-piperidyl)-3-methyl-2-oxo-benzimidazol-5-yl]-1-piperidyl]acetic acid (1, 120 mg, 233.26 μmol, TFA salt) in DMF (2.5 mL) at RT. The resulting mixture was stirred at RT for 4 h. Subsequently, 5-[7-(2-aminoethoxy)-1-fluoro-3-hydroxy-2-naphthyl]-1,1-dioxo-1,2,5-thiadiazolidin-3-one (2, 120 mg, 338 μmol) was added to the reaction, and the resulting mixture was stirred at RT for 12 h. The reaction was concentrated, and the residue was purified by reverse phase HPLC [Purification method: Column: X bridge (150×19 mm), 5 um; Mobile phase A: 0.1% TFA in MQ-water; Mobile phase B: Acetonitrile] to yield 2-[4-[1-(2,6-dioxo-3-piperidyl)-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 4, 53 mg, 59.33 μmol, 18%, TFA salt) as an off-white powder.

LCMS (ES+): m/z 737.76 [M+H]⁺

¹H NMR (400 MHZ, DMSO-d₆) δ 11.09 (s, 1H), 9.59 (s, 1H), 8.86 (s, 1H), 7.69 (dd, J=9.2, 1.5 Hz, 1H), 7.28-7.22 (m, 2H), 7.17-7.12 (m, 2H), 7.09-7.00 (m, 3H), 6.92 (d, J=8.2 Hz, 1H), 5.34 (dd, J=12.8, 5.4 Hz, 1H), 4.18 (dd, J=5.4 Hz, 2H), 4.10 (s, 2H), 4.01-3.92 (m, 2H), 3.65-3.57 (m, 2H), 3.56-3.49 (m, 2H), 3.34 (s, 3H), 3.18-3.08 (m, 2H), 2.96-2.79 (m, 2H), 2.76-2.59 (m, 2H), 2.11-1.91 (m, 5H).

### 2-[4-[3-(2,6-dioxo-3-piperidyl)-2-oxo-1,3-benzoxazol-6-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 5)

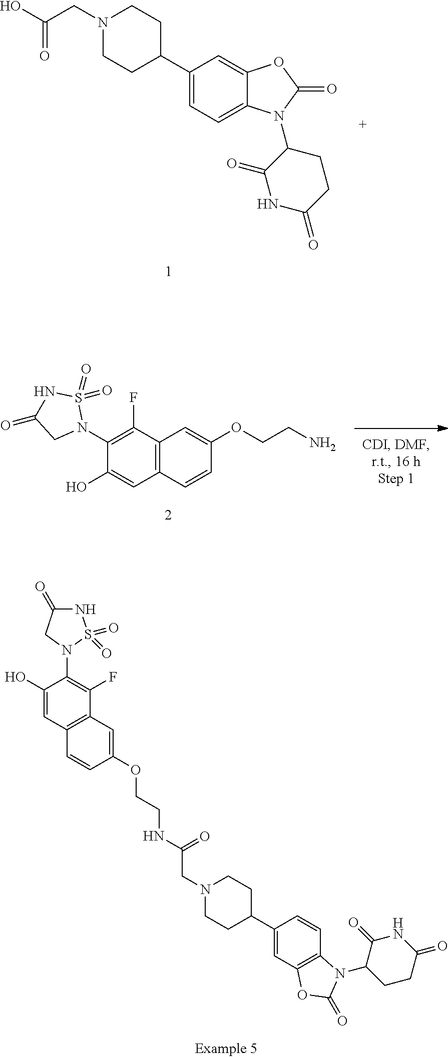 [SMILES: O=C(O)CN1CCC(c2ccc3c(c2)oc(=O)n3C2CCC(=O)NC2=O)CC1 ; NCCOc1ccc2cc(O)c(N3CC(=O)NS3(=O)=O)c(F)c2c1 ; O=C(CN1CCC(c2ccc3c(c2)oc(=O)n3C2CCC(=O)NC2=O)CC1)NCCOc1ccc2cc(O)c(N3CC(=O)NS3(=O)=O)c(F)c2c1]

### Step 1: 2-[4-[3-(2,6-dioxo-3-piperidyl)-2-oxo-1,3-benzoxazol-6-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 5)

Into a 10 mL single neck round bottom flask containing a well-stirred solution of 2-[4-[3-(2,6-dioxo-3-piperidyl)-2-oxo-1,3-benzoxazol-6-yl]-1-piperidyl]acetic acid (1, 90 mg, 0.232 mmol, TFA salt) in DMF (2 mL) was added 1,1′-carbonyldiimidazole (94.18 mg, 0.580 mmol) at RT, and the reaction was stirred for 4 h. 5-[7-(2-aminoethoxy)-1-fluoro-3-hydroxy-2-naphthyl]-1,1-dioxo-1,2,5-thiadiazolidin-3-one (2, 109.23 mg, 0.278 mmol) was then added to the reaction, and the resulting suspension was stirred at ambient temperature for 12 h. The reaction mixture was diluted with water (2 mL) and concentrated under reduced pressure to provide a residue, which was purified by reverse phase prep HPLC [Purification method: Column: Sunfire C18 (19×150 mm) 5.0 μm; Mobile phase A: 0.1% TFA in water; Mobile phase B: Acetonitrile) to afford 2-[4-[3-(2,6-dioxo-3-piperidyl)-2-oxo-1,3-benzoxazol-6-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 5, 45 mg, 0.049 mmol, 21% yield, TFA salt) as an off white solid.

LCMS (ES+): m/z 723.1 [M−H]⁻

¹H NMR (400 MHZ, DMSO-d₆) δ 11.21 (s, 1H), 9.55 (s, 1H), 8.84 (s, 1H), 7.69 (d, J=9.1 Hz, 1H), 7.29 (s, 1H), 7.25-7.19 (m, 2H), 7.14 (dd, J=9.0, 2.5 Hz, 1H), 7.09 (d, J=8.5 Hz, 1H), 7.03 (s, 1H), 5.35 (dd, J=13.0, 5.4 Hz, 1H), 4.18 (t, J=5.4 Hz, 2H), 4.10 (s, 2H), 3.96 (s, 2H), 3.65-3.57 (m, 2H), 3.56-3.46 (m, 2H), 3.19-3.06 (m, 2H), 2.96-2.79 (m, 2H), 2.74-2.61 (m, 2H), 2.20-2.09 (m, 1H), 2.06-1.90 (m, 4H).

### N-(2-((7-(1,1-dioxido-4-oxo-1,2,5-thiadiazolidin-2-yl)-8-fluoro-6-hydroxynaphthalen-2-yl)oxy)ethyl)-3-(4-(1-(2,6-dioxopiperidin-3-yl)-3-methyl-2-oxo-2,3-dihydro-1H-benzo[d]imidazol-5-yl)piperidin-1-yl)propanamide (Example 6)

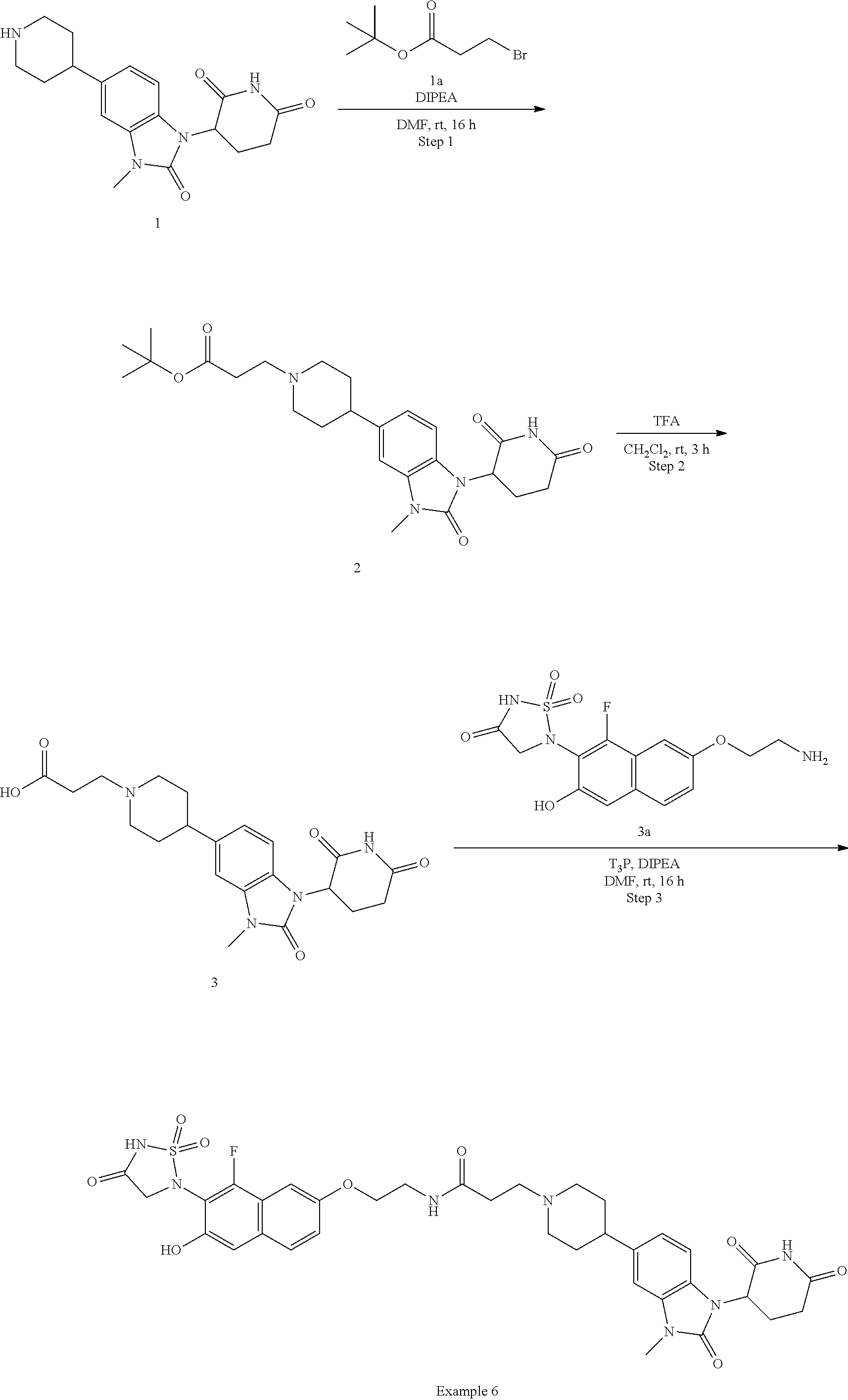

### Step 1: tert-butyl 3-(4-(1-(2,6-dioxopiperidin-3-yl)-3-methyl-2-oxo-2,3-dihydro-1H-benzo[d]imidazol-5-yl)piperidin-1-yl)propanoate (2)

Into a 50 mL single neck round bottom flask containing a well-stirred solution of 3-[3-methyl-2-oxo-5-(4-piperidyl)benzimidazol-1-yl]piperidine-2,6-dione (1, 300 mg, 876.19 μmol) in anhydrous DMF (10 mL) were added DIPEA (339.72 mg, 2.63 mmol, 457.85 μL) and tert-butyl 3-bromopropanoate (1a, 219.83 mg, 1.05 mmol). After 16 h, the mixture was concentrated under reduced pressure and purified by reverse-phase column chromatography [Redisep ISCO C18 (30 g) column; Mobile phase A: 0.1% TFA in MQ-water; Mobile phase B: Acetonitrile] to afford tert-butyl 3-[4-[1-(2,6-dioxo-3-piperidyl)-3-methyl-2-oxo-benzimidazol-5-yl]-1-piperidyl]propanoate (2, 160 mg, 257.26 μmol, 29% yield, TFA salt) as an off-white solid.

LCMS (ES+): m/z 471.2 [M+H]⁺

### Step 2: 3-(4-(1-(2,6-dioxopiperidin-3-yl)-3-methyl-2-oxo-2,3-dihydro-1H-benzo[d]imidazol-5-yl)piperidin-1-yl)propanoic acid (3)

Into a 25 mL single neck round bottom flask containing a well-stirred solution tert-butyl 3-[4-[1-(2,6-dioxo-3-piperidyl)-3-methyl-2-oxo-benzimidazol-5-yl]-1-piperidyl]propanoate (2, 160 mg, 0.340 mmol) in anhydrous DCM (4 mL) was added TFA (193.84 mg, 1.70 mmol, 0.13 mL) at RT and the resulting solution was stirred for 3 h. The reaction mixture was concentrated under reduced pressure and the residue azeotroped with toluene (2×5 mL) and triturated with MTBE (2×10 mL), filtered and dried to afford 3-[4-[1-(2,6-dioxo-3-piperidyl)-3-methyl-2-oxo-benzimidazol-5-yl]-1-piperidyl]propanoic acid (3, 140 mg, 236.38 μmol, 70% yield, TFA salt) as a grey solid.

LCMS (ES+): m/z 414.9 [M+H]⁺

### Step 3: N-(2-((7-(1,1-dioxido-4-oxo-1,2,5-thiadiazolidin-2-yl)-8-fluoro-6-hydroxynaphthalen-2-yl)oxy)ethyl)-3-(4-(1-(2,6-dioxopiperidin-3-yl)-3-methyl-2-oxo-2,3-dihydro-1H-benzo[d]imidazol-5-yl)piperidin-1-yl)propanamide (Example 6)

Into a 25 mL single neck round bottom flask containing a well-stirred solution of 3-[4-[1-(2,6-dioxo-3-piperidyl)-3-methyl-2-oxo-benzimidazol-5-yl]-1-piperidyl]propanoic acid (3, 60 mg, 113.53 μmol, TFA salt) in anhydrous DMF (3.5 mL) were added DIPEA (44.02 mg, 340.60 μmol, 59.33 μL), propylphosphonic anhydride solution (50 wt. % in EtOAc) (0.1 mL, 54.19 mg, 170.30 μmol) and 5-[7-(2-aminoethoxy)-1-fluoro-3-hydroxy-2-naphthyl]-1,1-dioxo-1,2,5-thiadiazolidin-3-one (3a, 48.9 mg, 124.89 μmol, HCl salt) at 0° C. The resulting mixture was stirred at RT for 16 h. The reaction mixture was diluted with water (2 mL), concentrated under reduced pressure and purified by reverse phase preparatory HPLC [Purification method: X-BRIDGE C18 (19×150 mm) 5.0 μm; Mobile phase A: 0.1% TFA in water; Mobile phase B: Acetonitrile) to afford 3-[4-[1-(2,6-dioxo-3-piperidyl)-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]propanamide (Example 6, 13 mg, 13.99 μmol, 12% yield, TFA salt) as an off-white solid.

LCMS (ES+): m/z 752.3 [M+H]⁺

¹H NMR (400 MHZ, DMSO-d₆) δ 11.10 (s, 1H), 10.13 (s, 1H), 9.64 (s, 1H), 8.57-8.48 (m, 1H), 7.68 (d, J=9.0 Hz, 1H), 7.23-7.20 (m, 1H), 7.15 (dd, J=9.0, 2.5 Hz, 1H), 7.08-7.02 (m, 3H), 6.92-6.84 (m, 1H), 5.36 (dd, J=12.7, 5.3 Hz, 1H), 4.14 (t, J=6.4 Hz, 2H), 4.11 (s, 1H), 3.56-3.49 (m, 4H), 3.36-3.30 (m, 5H), 3.10-2.99 (m, 2H), 2.97-2.79 (m, 2H), 2.77-2.59 (m, 5H), 2.08-1.91 (m, 4H).

### N-(2-((7-(1,1-dioxido-4-oxo-1,2,5-thiadiazolidin-2-yl)-8-fluoro-6-hydroxynaphthalen-2-yl)oxy)ethyl)-2-(4-(1-(2,6-dioxopiperidin-3-yl)-2-oxo-1,2-dihydrobenzo[cd]indol-6-yl)piperidin-1-yl)acetamide (Example 7)

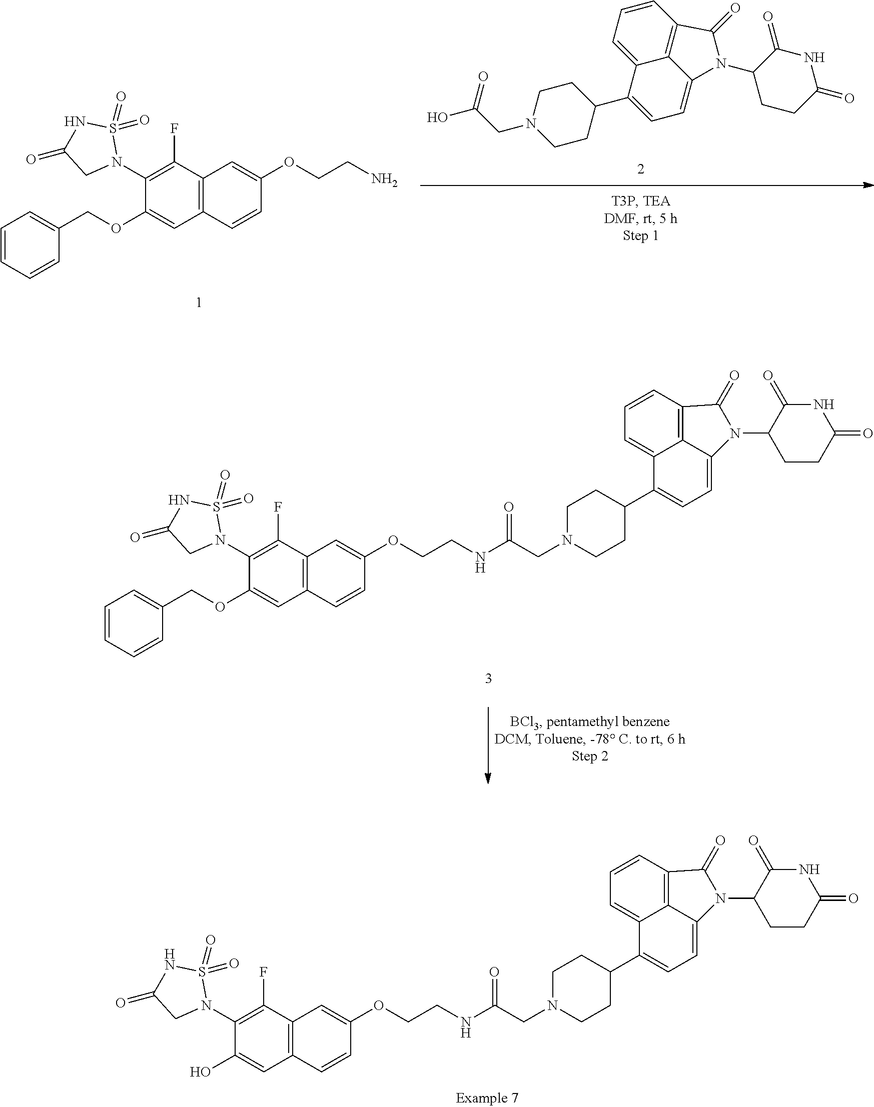 [SMILES: NCCOc1ccc2cc(OCc3ccccc3)c(N3CC(=O)NS3(=O)=O)c(F)c2c1 ; O=C(O)CN1CCC(c2ccc3c4c(cccc24)C(=O)N3C2CCC(=O)NC2=O)CC1 ; O=C(CN1CCC(c2ccc3c4c(cccc24)C(=O)N3C2CCC(=O)NC2=O)CC1)NCCOc1ccc2cc(OCc3ccccc3)c(N3CC(=O)NS3(=O)=O)c(F)c2c1 ; O=C(CN1CCC(c2ccc3c4c(cccc24)C(=O)N3C2CCC(=O)NC2=O)CC1)NCCOc1ccc2cc(O)c(N3CC(=O)NS3(=O)=O)c(F)c2c1]

### Step 1: N-(2-((6-(benzyloxy)-7-(1,1-dioxido-4-oxo-1,2,5-thiadiazolidin-2-yl)-8-fluoronaphthalen-2-yl) oxy)ethyl)-2-(4-(1-(2,6-dioxopiperidin-3-yl)-2-oxo-1,2-dihydrobenzo[cd]indol-6-yl)piperidin-1-yl) acetamide (3)

Into a 25 mL single neck round bottom flask containing a well-stirred solution of 5-[7-(2-aminoethoxy)-3-benzyloxy-1-fluoro-2-naphthyl]-1,1-dioxo-1,2,5-thiadiazolidin-3-one (1, 270 mg, 482.59 μmol, TFA salt) and 2-[4-[1-(2,6-dioxo-3-piperidyl)-2-oxo-benzo[cd]indol-6-yl]-1-piperidyl]acetic acid (2, 258.41 mg, 482.59 μmol, TFA salt) in anhydrous DMF (3.5 mL) were added propylphosphonic anhydride (T3P) (≥50 wt. % in ethyl acetate) (307.10 mg, 965.17 μmol, 680 μL) and DIPEA (742.00 mg, 5.74 mmol, 1 mL) at RT. The resulting mixture was stirred at RT for 5 h. The solvent was removed from the reaction mixture, and the residue was purified by reverse phase prep HPLC [Purification method: Column: X-Select C18 (10×150 mm) 5 μm, mobile phase: 0.1% TFA in water and MeCN] to afford N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-2-oxo-benzo[cd]indol-6-yl]-1-piperidyl] acetamide (3, 140 mg, 133.92 μmol, 28% yield, TFA salt) as light yellow solid.

LCMS (ES+): m/z 849.7 [M+H]⁺

### Step-2: N-(2-((7-(1,1-dioxido-4-oxo-1,2,5-thiadiazolidin-2-yl)-8-fluoro-6-hydroxynaphthalen-2-yl)oxy) ethyl)-2-(4-(1-(2,6-dioxopiperidin-3-yl)-2-oxo-1,2-dihydrobenzo[cd]indol-6-yl)piperidin-1-yl)acetamide (Example 7)

Into a 25 mL single neck round bottom flask containing a well-stirred solution of N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-2-oxo-benzo[cd]indol-6-yl]-1-piperidyl]acetamide (3, 100 mg, 103.85 μmol, TFA salt) and pentamethylbenzene (87.32 mg, 589.00 μmol, 95.22 μL) in anhydrous DCM (7.5 mL) and toluene (7.5 mL) was added boron trichloride (1.0 M in methylene chloride) (276.05 mg, 2.36 mmol, 2.36 mL) at −78° C. The reaction mixture was stirred at ambient temperature for 6 h. The reaction was quenched with 5% MeOH in DCM (3 mL) at −78° C., and excess solvent was removed under reduced pressure. The residue was washed with MTBE (20 mL) and purified by reverse phase prep HPLC [Purification method: Column: Agilent C18 (50×21.2) 5 micron; Mobile phase A: 10 mM NH₄OAC in water and Mobile phase B: MeCN] to afford 2-[4-[1-(2,6-dioxo-3-piperidyl)-2-oxo-benzo[cd]indol-6-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 7, 20 mg, 26.19 μmol, 22% yield) as yellow solid.

LCMS (ES+): m/z 759.0 [M+H]⁺

¹H NMR (400 MHZ, DMSO-d₆) δ 11.14 (s, 1H), 9.81 (s, 1H), 9.55 (s, 1H), 8.90 (s, 1H), 8.47 (d, J=8.4 Hz, 1H), 8.13 (d, J=7.0 Hz, 1H), 7.89 (t, J=7.6 Hz, 1H), 7.69 (d, J=9.0 Hz, 1H), 7.34 (d, J=7.5 Hz, 1H), 7.26-7.23 (m, 1H), 7.18-7.12 (m, 2H), 7.03 (s, 1H), 5.45 (dd, J=12.9, 5.4 Hz, 1H), 4.24-4.17 (m, 2H), 4.09 (s, 2H), 4.01 (s, 2H), 3.68-3.54 (m, 5H), 3.03-2.90 (m, 1H), 2.82-2.72 (m, 1H), 2.71-2.61 (m, 2H), 2.26-1.94 (m, 6H).

### 2-[4-[3-(2,6-dioxo-3-piperidyl)-1-methyl-indazol-6-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 8)

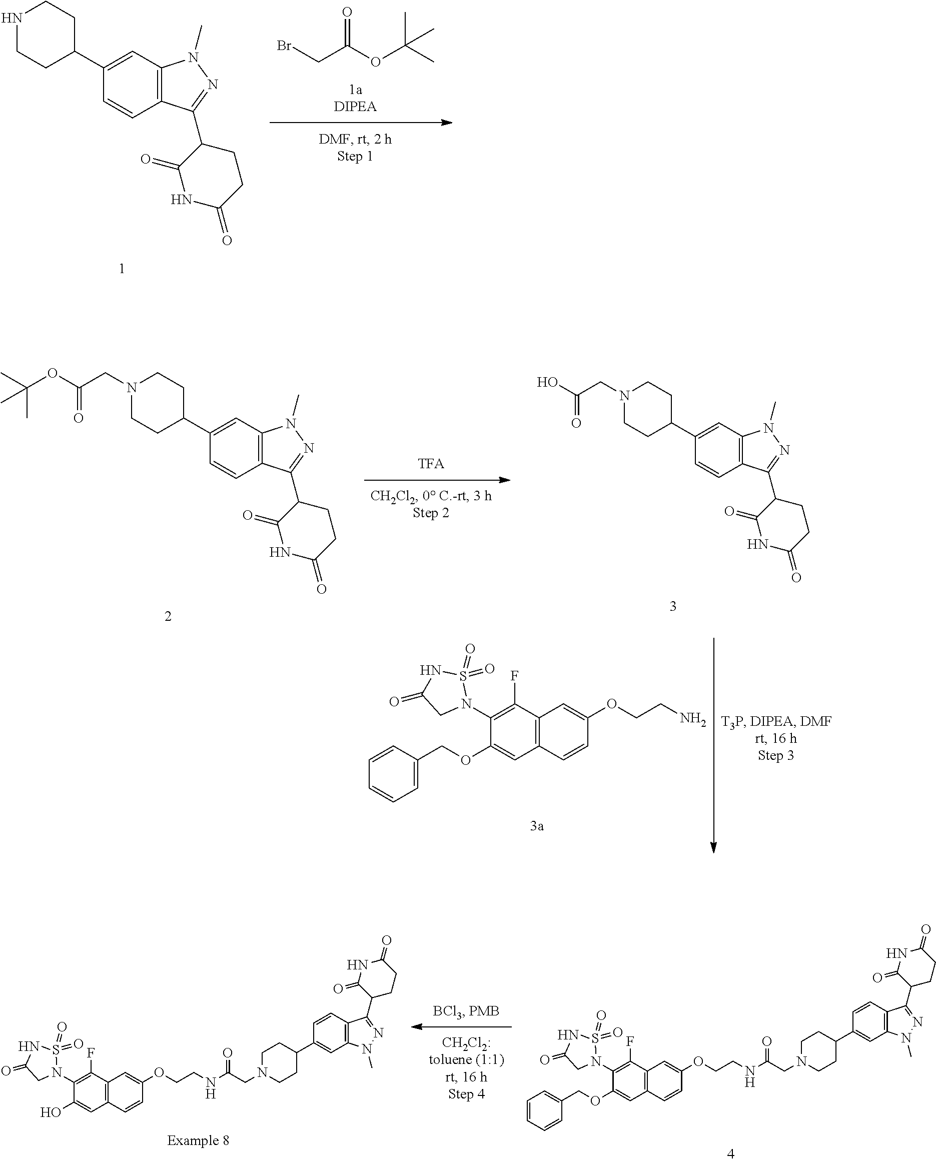

### Step 1: tert-butyl 2-[4-[3-(2,6-dioxo-3-piperidyl)-1-methyl-indazol-6-yl]-1-piperidyl] acetate (2)

Into a 50 mL single neck round bottom flask containing a well-stirred solution of 3-[1-methyl-6-(4-piperidyl)indazol-3-yl]piperidine-2,6-dione (1, 500 mg, 1.14 mmol. TFA salt) in anhydrous DMF (7 mL) were added DIPEA (440.19 mg, 3.41 mmol, 593.24 μL) and tert-butyl bromoacetate (1a. 221.44 mg, 1.14 mmol, 166.50 μL). The reaction was stirred at ambient temperature for 2 h. The reaction mixture was poured into ice-cold water (20 mL), and the aqueous layer was extracted with DCM (2×30 mL). The combined organic layer was washed with brine (15 mL), dried over anhydrous Na₂SO₄, and filtered. The residue was triturated with diethyl ether, filtered, and dried to afford tert-butyl 2-[4-[3-(2,6-dioxo-3-piperidyl)-1-methyl-indazol-6-yl]-1-piperidyl]acetate (2, 350) mg, 555.35 μmol, 49% yield) as an off-white solid.

LCMS (ES+): m/z 440.9 [M+H]⁺

### Step 2: 2-[4-[3-(2,6-dioxo-3-piperidyl)-1-methyl-indazol-6-yl]-1-piperidyl]acetic acid (3)

Into a 25 mL single neck round bottom flask containing a well-stirred solution of tert-butyl 2-[4-[3-(2,6-dioxo-3-piperidyl)-1-methyl-indazol-6-yl]-1-piperidyl]acetate (2, 350 mg, 794.49 μmol) in anhydrous DCM (5 mL) was added TFA (905.90 mg, 7.94 mmol, 612.10 μL) at 0° C. The reaction was stirred for 3 h at ambient temperature. The reaction mixture was concentrated under reduced pressure, and the residue was azeotroped with toluene (2×15 mL), triturated with diethyl ether (20 mL), filtered, and dried to afford 2-[4-[3-(2,6-dioxo-3-piperidyl)-1-methyl-indazol-6-yl]-1-piperidyl]acetic acid (3, 300 mg, 505.87 μmol, 64% yield, TFA salt) as an off-white solid. LCMS (ES+): m/z 385.0 [M+H]⁺

### Step 3: N-[2-[[6-benzyloxy-8-fluoro-7-(1,1,4-trioxo-1,2,5-thiadiazolidin-2-yl)-2-naphthyl]oxy]ethyl]-2-[4-[3-(2,6-dioxo-3-piperidyl)-1-methyl-indazol-6-yl]-1-piperidyl]acetamide (4)

Into a 25 mL single neck round bottom flask containing a well-stirred solution of 2-[4-[3-(2,6-dioxo-3-piperidyl)-1-methyl-indazol-6-yl]-1-piperidyl]acetic acid (3, 300 mg, 601.86 μmol, TFA salt) in anhydrous DMF (5 mL) were added 5-[7-(2-aminoethoxy)-3-benzyloxy-1-fluoro-2-naphthyl]-1,1-dioxo-1,2,5-thiadiazolidin-3-one (3a, 336.73 mg, 601.86 μmol, TFA salt). 1-propanephosphonic anhydride (50% in EtOAc) (383.00 mg, 1.20 mmol) and DIPEA (233.36 mg. 1.81 mmol, 314.50 μL). The resulting mixture was stirred at ambient temperature for 16 h. After completion of the reaction, the volatiles were removed under reduced pressure, and the residue was purified by reverse phase preparative HPLC [Column: YMC C18 (19×150) mm. 5 micron; Mobile phase A: 0.1% TFA in water and Mobile phase B: MeCN] to obtain N-[2-[[6-benzyloxy-8-fluoro-7-(1,1,4-trioxo-1,2,5-thiadiazolidin-2-yl)-2-naphthyl]oxy]ethyl]-2-[4-[3-(2,6-dioxo-3-piperidyl)-1-methyl-indazol-6-yl]-1-piperidyl] acetamide (4, 160 mg, 167.62 μmol, 28% yield, TFA salt) as an off-white solid.

LCMS (ES+): m/z 811.8 [M+H]⁺

### Step 4: 2-[4-[3-(2,6-dioxo-3-piperidyl)-1-methyl-indazol-6-yl]-1-piperidyl]-N-[2-[[8-fluoro-6-hydroxy-7-(1,1,4-trioxo-1,2,5-thiadiazolidin-2-yl)-2-naphthyl]oxy]ethyl] acetamide (Example 8)

Into a 50 mL single neck round bottom flask containing well-stirred solution of N-[2-[[6-benzyloxy-8-fluoro-7-(1,1,4-trioxo-1,2,5-thiadiazolidin-2-yl)-2-naphthyl]oxy]ethyl]-2-[4-[3-(2,6-dioxo-3-piperidyl)-1-methyl-indazol-6-yl]-1-piperidyl]acetamide (4, 160 mg, 172.81 μmol, TFA salt) in a 1:1 solution of anhydrous DCM (4 mL) and anhydrous toluene (4 mL), was added pentamethylbenzene (128.09 mg, 864.03 μmol, 139.68 μL). The reaction mixture was cooled to −78° C. and BCl₃ (1.0 M in methylene chloride) (303.71 mg, 2.59 mmol) was slowly added to the reaction mixture. The resulting mixture was stirred at ambient temperature for 16 h. The reaction was quenched with 5% MeOH in DCM (6 mL) at −78° C. and the volatiles were removed under reduced pressure, and the residue was purified by reverse phase preparative HPLC [Column: Redisep C18 (19×150) mm, 5 micron; Mobile phase A: 0.1% TFA in water and Mobile phase B: MeCN] to obtain 2-[4-[3-(2,6-dioxo-3-piperidyl)-1-methyl-indazol-6-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 8, 14.5 mg, 16.36 μmol, 9% yield, TFA salt) as an off-white solid.

LCMS (ES+): m/z 722.2 [M+H]⁺

¹H NMR (300 MHz, DMSO-d₆) δ 10.89 (s, 1H), 9.71 (s, 1H), 9.55 (s, 1H), 8.88 (s, 1H), 7.73-7.64 (m, 2H), 7.41 (s, 1H), 7.26-7.21 (m, 1H), 7.19-7.12 (m, 1H), 7.07-7.01 (m, 2H), 4.34 (dd, J=9.8, 5.0 Hz, 1H), 4.23-4.15 (m, 2H), 4.10 (s, 2H), 3.98 (s, 4H), 3.59 (dd, J=14.2, 8.6 Hz, 3H), 2.95 (s, 2H), 2.75-2.57 (m, 3H), 2.41-2.26 (m, 3H), 2.23-1.97 (m, 5H).

### 2-[4-[1-(2,6-dioxo-3-piperidyl)-3-methyl-2-oxo-benzimidazol-4-yl]oxy-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 9)

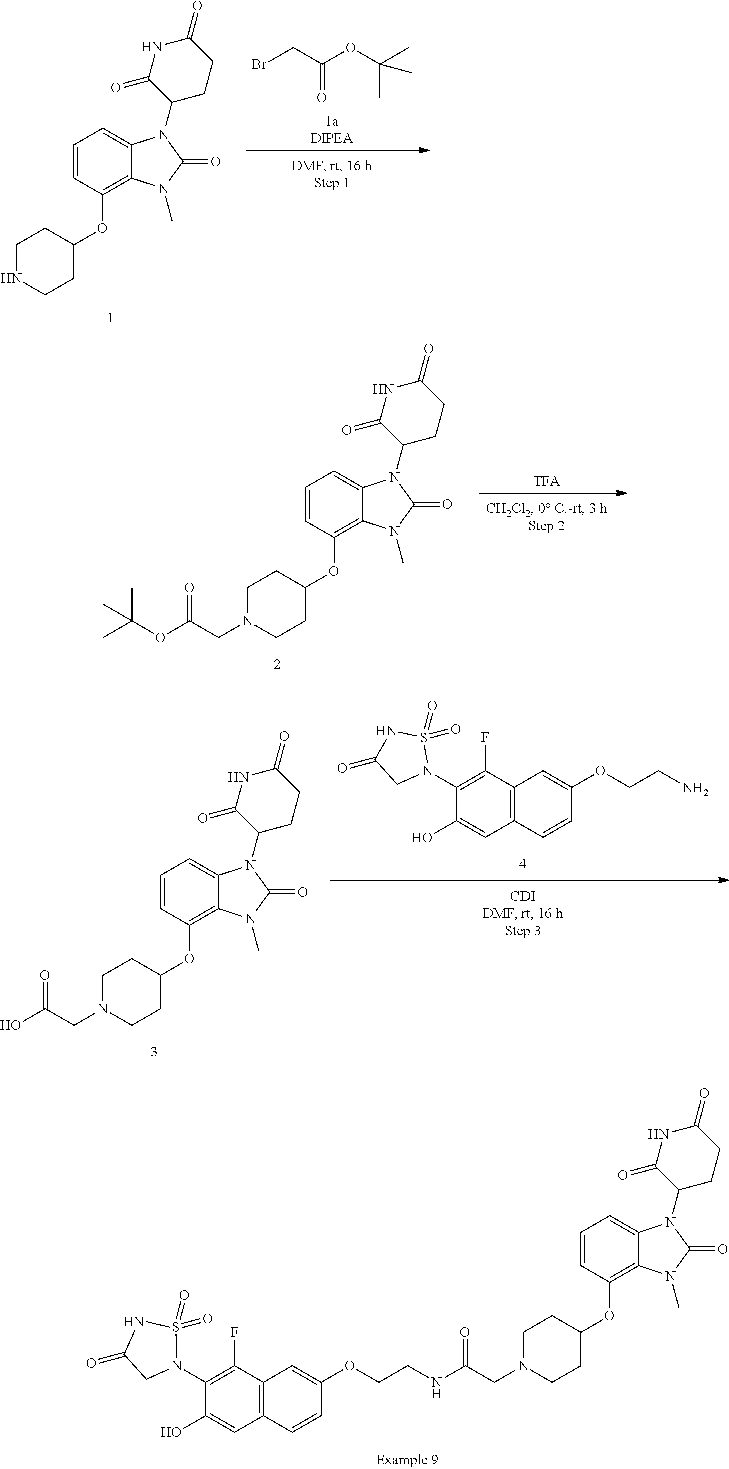

### Step 1: tert-butyl 2-[4-[1-(2,6-dioxo-3-piperidyl)-3-methyl-2-oxo-benzimidazol-4-yl]oxy-1-piperidyl]acetate (2)

Into a 50 mL single neck round bottom flask containing a well-stirred solution of 3-[3-methyl-2-oxo-4-(4-piperidyloxy)benzimidazol-1-yl]piperidine-2,6-dione (1, 120 mg, 334.83 μmol) in anhydrous DMF (5 mL) were added tert-butyl 2-bromoacetate (1a, 71.84 mg, 368.31 μmol, 54.02 μL) and DIPEA (129.82 mg, 1.00 mmol, 174.96 μL). After 16 h, the reaction mixture was poured into ice-cold water (20 mL). The precipitate was collected by filtration and dried under vacuum to afford tert-butyl 2-[4-[1-(2,6-dioxo-3-piperidyl)-3-methyl-2-oxo-benzimidazol-4-yl]oxy-1-piperidyl]acetate (2, 110 mg, 229.99 μmol, 69% yield) as a pale-yellow solid.

LCMS (ES+): m/z 473.2 [M+H]⁺

### Step 2: 2-[4-[1-(2,6-dioxo-3-piperidyl)-3-methyl-2-oxo-benzimidazol-4-yl]oxy-1-piperidyl]acetic acid (3)

Into a 50 mL single neck round bottom flask containing a well-stirred solution of tert-butyl 2-[4-[1-(2,6-dioxo-3-piperidyl)-3-methyl-2-oxo-benzimidazol-4-yl]oxy-1-piperidyl]acetate (2, 110 mg, 232.79 μmol) in anhydrous DCM (2 mL) was added TFA (132.72 mg, 1.16 mmol, 89.67 μL) at 0° C. After stirring at RT for 3 h, the reaction mixture was concentrated under reduced pressure and the residue azeotroped with toluene (2×15 mL) and triturated with diethyl ether (20 mL) to afford 2-[4-[1-(2,6-dioxo-3-piperidyl)-3-methyl-2-oxo-benzimidazol-4-yl]oxy-1-piperidyl]acetic acid (3, 110 mg, 192.30 μmol, 83% yield, TFA salt) as an off-white solid.

LCMS (ES+): m/z 416.90 [M+H]⁺

### Step 3: 2-[4-[1-(2,6-dioxo-3-piperidyl)-3-methyl-2-oxo-benzimidazol-4-yl]oxy-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 9)

Into a 25 mL single neck round bottom flask containing a well-stirred solution of 2-[4-[1-(2,6-dioxo-3-piperidyl)-3-methyl-2-oxo-benzimidazol-4-yl]oxy-1-piperidyl]acetic acid (3, 100 mg, 188.52 μmol, TFA salt) in anhydrous DMF (3 mL) was added CDI (61.14 mg, 377.04 μmol) at ambient temperature under nitrogen atmosphere and stirred for 4 h. Subsequently, 5-[7-(2-aminoethoxy)-1-fluoro-3-hydroxy-2-naphthyl]-1,1-dioxo-1,2,5-thiadiazolidin-3-one (4, 81.25 mg, 207.37 μmol, HCl salt) was added to the reaction mixture. After 12 h, the reaction mixture was concentrated under reduced pressure and purified by reverse-phase preparative HPLC [Column: XBRIDGE C18 (19×150) mm, 5 micron; Mobile phase A: 0.1% TFA in water and Mobile phase B: MeCN] to obtain 2-[4-[1-(2,6-dioxo-3-piperidyl)-3-methyl-2-oxo-benzimidazol-4-yl]oxy-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 9, 40 mg, 44.27 μmol, 23% yield, TFA salt) as an off-white solid.

LCMS (ES+): m/z 754.3 [M+H]⁺

¹H NMR (400 MHZ, DMSO-d₆) δ 11.09 (s, 1H), 9.81 (s, 1H), 9.54 (s, 1H), 8.80 (s, 1H), 7.68 (d, J=9.0 Hz, 1H), 7.22 (d, J=2.5 Hz, 1H), 7.13 (dd, J=9.0, 2.5 Hz, 1H), 7.03 (s, 1H), 6.97 (t, J=8.2 Hz, 1H), 6.86-6.74 (m, 2H), 5.33 (dd, J=12.8, 5.4 Hz, 1H), 4.90-4.58 (m, 1H), 4.17 (t, J=5.4 Hz, 2H), 4.09 (s, 2H), 4.03-3.89 (m, 2H), 3.66-3.50 (m, 5H), 2.95-2.83 (m, 1H), 2.76-2.58 (m, 3H), 2.28-1.91 (m, 5H).

### 2-[4-[4-[(2,6-dioxo-3-piperidyl)amino]-2-fluoro-phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 10)

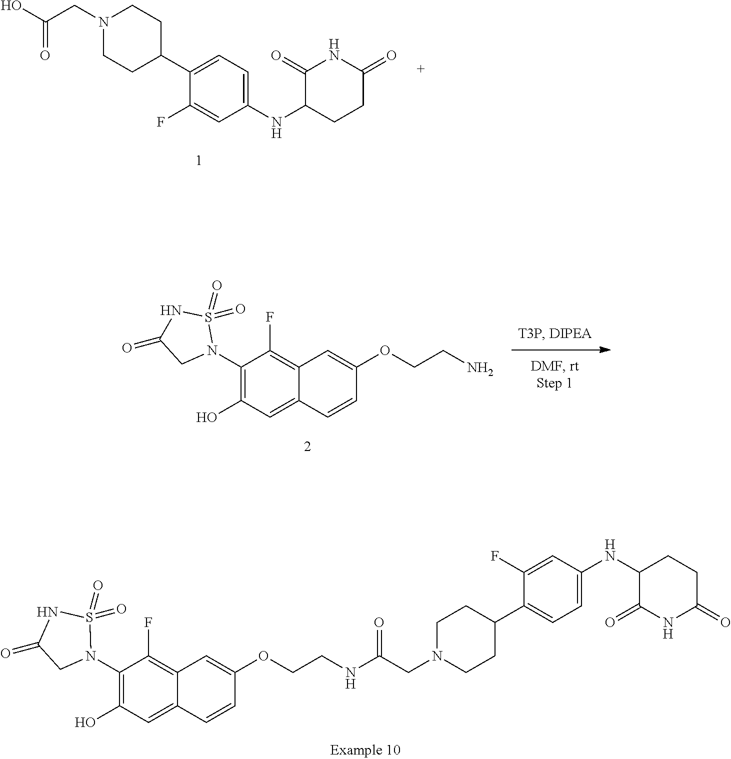 [SMILES: O=C(O)CN1CCC(c2ccc(NC3CCC(=O)NC3=O)cc2F)CC1 ; NCCOc1ccc2cc(O)c(N3CC(=O)NS3(=O)=O)c(F)c2c1 ; O=C(CN1CCC(c2ccc(NC3CCC(=O)NC3=O)cc2F)CC1)NCCOc1ccc2cc(O)c(N3CC(=O)NS3(=O)=O)c(F)c2c1]

### Step 1: 2-[4-[4-[(2,6-dioxo-3-piperidyl)amino]-2-fluoro-phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 10)

2-[4-[4-[(2,6-dioxo-3-piperidyl)amino]-2-fluoro-phenyl]-1-piperidyl]acetic acid (1, 24.54 mg, 67.54 μmol) and 5-[7-(2-aminoethoxy)-1-fluoro-3-hydroxy-2-naphthyl]-1,1-dioxo-1,2,5-thiadiazolidin-3-one (2, 20 mg, 56.28 μmol) in DMF (500 μL) were treated with DIPEA (36.37 mg, 281.42 μmol, 49.02 μL). The mixture was cooled to 0° C. before addition of propylphosphonic anhydride (T3P) (50% in EtOAc) (39.40 mg, 61.91 μmol, 36.82 μL). The mixture was allowed to come to RT and was stirred for 16 h. The mixture was purified via reverse phase chromatography, eluting with 5-100% MeCN in H₂O (with 0.1% TFA modifier) to afford 2-[4-[4-[(2,6-dioxo-3-piperidyl)amino]-2-fluoro-phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 10, 7.17 mg, 8.44 μmol, 15% yield) as a gray solid.

LCMS (ES+): m/z 701.3 [M+H]⁺

¹H NMR (400 MHZ, DMSO-d₆) δ 10.79 (s, 1H), 9.67 (s, 1H), 9.54 (s, 1H), 8.86 (t, J=5.2 Hz, 1H), 7.70 (d, J=9.0 Hz, 1H), 7.23 (d, J=2.5 Hz, 1H), 7.14 (dd, J=9.0, 2.5 Hz, 1H), 7.04 (s, 1H), 6.99-6.91 (m, 1H), 6.52-6.42 (m, 2H), 6.10 (s, 1H), 4.37-4.27 (m, 1H), 4.18 (t, J=5.3 Hz, 2H), 4.11 (s, 2H), 3.95 (d, J=4.8 Hz, 2H), 3.61 (q, J=5.5 Hz, 2H), 3.52 (d, J=10.4 Hz, 1H), 3.21-3.09 (m, 1H), 2.96-2.84 (m, 1H), 2.74 (ddd, J=17.4, 12.3, 5.4 Hz, 1H), 2.62-2.58 (m, 1H), 2.15-1.94 (m, 2H), 1.95-1.79 (m, 3H).

### 3-(7-(1,1-dioxido-4-oxo-1,2,5-thiadiazolidin-2-yl)-8-fluoro-6-hydroxynaphthalen-2-yl)-N-(1-(2-((2-(2,6-dioxopiperidin-3-yl)-1-oxoisoindolin-4-yl)oxy)acetyl)piperidin-4-yl)acrylamide (Example 11)

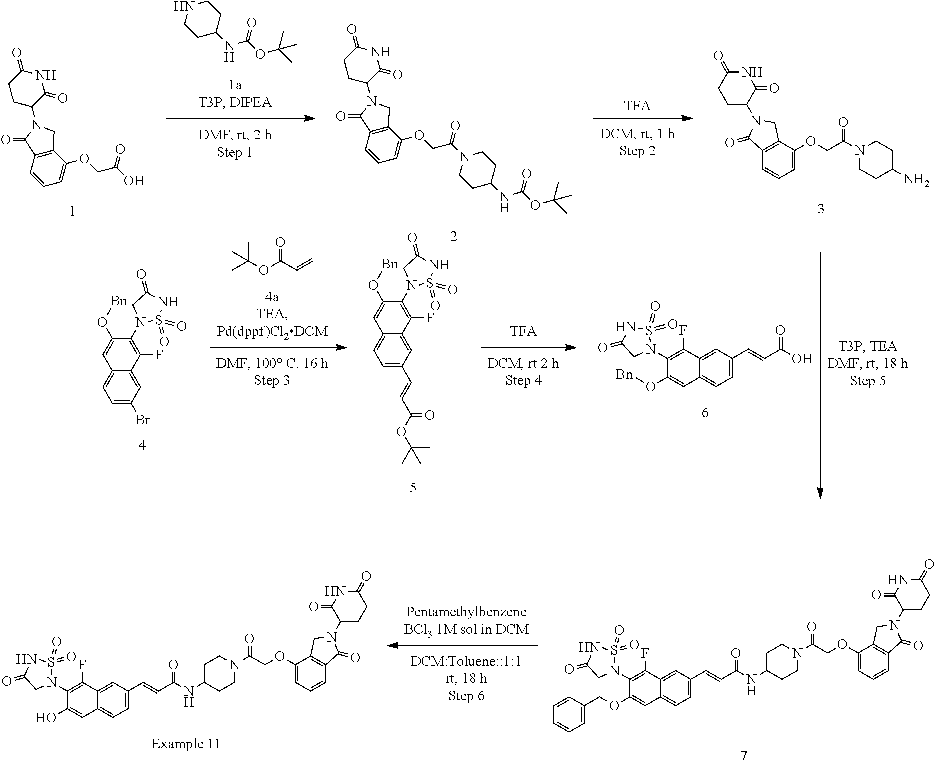

### Step 1: tert-butyl N-[1-[2-[2-(2,6-dioxo-3-piperidyl)-1-oxo-isoindolin-4-yl]oxyacetyl]-4-piperidyl]carbamate (2)

Into a 25 mL round bottom flask containing a well-stirred solution of 2-[2-(2,6-dioxo-3-piperidyl)-1-oxo-isoindolin-4-yl]oxyacetic acid (1, 340 mg, 1.07 mmol) in anhydrous DMF (4 mL) were added DIPEA (276.12 mg, 2.14 mmol, 372.14 μL) and T₃P (50% in EtOAc) (1 mL, 509.55 mg. 1.60 mmol). After 5 min. tert-butyl N-(4-piperidyl)carbamate (1a, 427.89 mg, 2.14 mmol) was added. After 2 h the volatiles were removed under reduced pressure and the residue purified by reverse phase column chromatography [Purification method: C18, Mobile Phase A: 0.1% TFA in water; Mobile phase B: MeCN] to obtain tert-butyl N-[1-[2-[2-(2,6-dioxo-3-piperidyl)-1-oxo-isoindolin-4-yl]oxyacetyl]-4-piperidyl]carbamate (2, 250 mg, 421.04 μmol, 39% yield) as a pale yellow solid. LCMS (ES+): m/z 401.2 [M-Boc+H]⁺

### Step 2: 3-[4-[2-(4-amino-1-piperidyl)-2-oxo-ethoxy]-1-oxo-isoindolin-2-yl]piperidine-2,6-dione (3)

Into a 10 mL round bottom flask containing a well-stirred solution of tert-butyl N-[1-[2-[2-(2,6-dioxo-3-piperidyl)-1-oxo-isoindolin-4-yl]oxyacetyl]-4-piperidyl]carbamate (2, 250 mg, 421.04 μmol) in DCM (3 mL) was added TFA (480.09 mg, 4.21 mmol, 324.38 μL) dropwise at 0° C. The reaction mixture was stirred at RT for 1 h. The volatiles were removed under reduced pressure. The residue was triturated with diethyl ether (2×10 mL), to yield 3-[4-[2-(4-amino-1-piperidyl)-2-oxo-ethoxy]-1-oxo-isoindolin-2-yl]piperidine-2,6-dione (3, 209 mg, 347.03 μmol, 82% yield, TFA salt) as a light yellow solid. LCMS (ES+): m/z 401.0 [M+H]⁺

### Step 3: tert-butyl (E)-3-(6-(benzyloxy)-7-(1,1-dioxido-4-oxo-1,2,5-thiadiazolidin-2-yl)-8-fluoronaphthalen-2-yl)acrylate (5)

Into a 25 mL pressure tube containing a well-stirred solution of tert-butyl acrylate (4a, 99.16 mg, 773.71 μmol, 112.30 μL) and 5-(3-(benzyloxy)-7-bromo-1-fluoronaphthalen-2-yl)-1,2,5-thiadiazolidin-3-one 1,1-dioxide (4, 90 mg, 193.43 μmol) in DMF (5 mL) was added triethylamine (97.86 mg, 967.13 μmol, 134.80 μL). The reaction mixture was degassed by bubbling nitrogen gas through for 5 min, then [1,1′-bis(diphenylphosphino) ferrocene]dichloropalladium(II) dichloromethane complex (15.80 mg, 19.34 μmol) was added and degassed for another 5 minutes. The tube was sealed, and the reaction mixture was stirred at 110° C. After 16 h, the reaction mixture was cooled to ambient temperature, filtered through Celite, washing with ethyl acetate (10) mL). The filtrate was concentrated under reduced pressure and the residue was diluted with EtOAc (50) mL) and washed with water (3×40 mL). The organic layer was dried over anhydrous sodium sulphate, filtered, and solvent removed under reduced pressure. The residue was purified by reverse phase column chromatography [mobile phase 0.1% TFA in Water and MeCN) to obtain tert-butyl (E)-3-(6-(benzyloxy)-7-(1,1-dioxido-4-oxo-1,2,5-thiadiazolidin-2-yl)-8-fluoronaphthalen-2-yl)acrylate (5, 60 mg, 111.19 μmol, 57% yield) as an off white solid. LCMS (ES−): m/z 511.0 [M−H]⁻

### Step 4: (E)-3-[6-benzyloxy-8-fluoro-7-(1,1,4-trioxo-1,2,5-thiadiazolidin-2-yl)-2-naphthyl] prop-2-enoic acid (6)

Into a 10 mL round bottom flask containing a well-stirred solution of tert-butyl (E)-3-(6-(benzyloxy)-7-(1,1-dioxido-4-oxo-1,2,5-thiadiazolidin-2-yl)-8-fluoronaphthalen-2-yl)acrylate (5, 110 mg, 214.61 μmol) in DCM (1.2 mL) was added TFA (244.71 mg, 2.15 mmol, 165.34 μL) dropwise at (° C.) The reaction mixture was stirred at RT for 2 h. The volatiles were removed under reduced pressure and the residue was triturated with diethyl ether (2×10 mL) and filtered to yield (E)-3-[6-benzyloxy-8-fluoro-7-(1,1,4-trioxo-1,2,5-thiadiazolidin-2-yl)-2-naphthyl]prop-2-enoic acid (6, 90 mg, 130.35 μmol, 61% yield) as a pale yellow solid.

LCMS (ES−): m/z 455.1 [M−H]⁻

### Step 5: 3-(6-(benzyloxy)-7-(1,1-dioxido-4-oxo-1,2,5-thiadiazolidin-2-yl)-8-fluoronaphthalen-2-yl)-N-(1-(2-((2-(2,6-dioxopiperidin-3-yl)-1-oxoisoindolin-4-yl)oxy) acetyl)piperidin-4-yl)acrylamide (7)

Into a 25 mL round bottom flask containing a well-stirred solution of (E)-3-[6-benzyloxy-8-fluoro-7-(1,1,4-trioxo-1,2,5-thiadiazolidin-2-yl)-2-naphthyl]prop-2-enoic acid (6, 80 mg, 175.27 μmol) in DMF (2 mL) was added triethylamine (53.21 mg, 525.81 μmol, 73.29 μL) and propylphosphonic anhydride (50% in EtOAc) (0.11 mL, 55.79 mg, 175.27 μmol). After 30 min, 3-(4-(2-(4-aminopiperidin-1-yl)-2-oxoethoxy)-1-oxoisoindolin-2-yl)piperidine-2,6-dione (3, 126.67 mg, 210.32 μmol, TFA salt) was added. The reaction mixture was stirred at ambient temperature 18 h. The volatiles were removed under reduced pressure and the residue purified by reverse phase prep HPLC [Purification method: Column: X-Bridge C18 (19×150 mm) 5.0 Micron; Mobile phase: 0.1% TFA in water; Mobile phase B: MeCN] to obtain 3-(6-(benzyloxy)-7-(1,1-dioxido-4-oxo-1,2,5-thiadiazolidin-2-yl)-8-fluoronaphthalen-2-yl)-N-(1-(2-((2-(2,6-dioxopiperidin-3-yl)-1-oxoisoindolin-4-yl)oxy)acetyl)piperidin-4-yl)acrylamide (7, 80 mg, 77.86 μmol, 44% yield, TFA salt) as a brown solid. LCMS (ES+): m/z 838.8 [M+H]⁺

### Step 6: 3-(7-(1,1-dioxido-4-oxo-1,2,5-thiadiazolidin-2-yl)-8-fluoro-6-hydroxynaphthalen-2-yl)-N-(1-(2-((2-(2,6-dioxopiperidin-3-yl)-1-oxoisoindolin-4-yl)oxy)acetyl)piperidin-4-yl)acrylamide (Example 11)

Into a 25 mL round bottom flask containing a well-stirred solution of 3-(6-(benzyloxy)-7-(1,1-dioxido-4-oxo-1,2,5-thiadiazolidin-2-yl)-8-fluoronaphthalen-2-yl)-N-(1-(2-((2-(2,6-dioxopiperidin-3-yl)-1-oxoisoindolin-4-yl)oxy)acetyl)piperidin-4-yl)acrylamide (7, 70 mg, 73.46 μmol, TFA salt) and pentamethylbenzene (108.90 mg, 734.62 μmol, 118.76 μL) in a mixture of DCM (2 mL) and toluene (2 mL) was added boron trichloride (1M in DCM) (0.73 mL, 86.08 mg, 734.62 μmol) dropwise at −78° C. The reaction mixture was then stirred at RT for 18 h. The reaction mixture was cooled to −78° C. and quenched by dropwise addition of 10:1 mixture of DCM and MeOH (5 mL). The volatiles were removed under reduced pressure and the residue purified by reverse phase prep HPLC [Purification method: Column: Sunfire (19×150 mm), 5 micron; Mobile phase A: 0.1% TFA in Water and Mobile phase B: MeCN] to obtain 3-(7-(1,1-dioxido-4-oxo-1,2,5-thiadiazolidin-2-yl)-8-fluoro-6-hydroxynaphthalen-2-yl)-N-(1-(2-((2-(2,6-dioxopiperidin-3-yl)-1-oxoisoindolin-4-yl)oxy) acetyl)piperidin-4-yl)acrylamide (Example 11, 8 mg, 9.01 μmol, 12% yield, TFA salt) as an off-white solid.

LCMS (ES+): m/z 749.1 [M+H]⁺

¹H NMR (400 MHZ, DMSO-d₆) δ 10.98 (s, 1H), 10.67 (s, 1H), 8.15 (d, J=7.5 Hz, 1H), 8.06 (s, 1H), 7.80 (d, J=8.6 Hz, 1H), 7.70 (d, J=8.9 Hz, 1H), 7.58 (d, J=15.7 Hz, 1H), 7.46 (t, J=7.8 Hz, 1H), 7.33 (d, J=7.6 Hz, 1H), 7.19-7.09 (m, 2H), 6.72 (d, J=15.7 Hz, 1H), 5.17-4.95 (m, 3H), 4.47-4.37 (m, 3H), 4.28 (d, J=17.4 Hz, 1H), 4.17 (d, J=13.0 Hz, 1H), 4.03-3.92 (m, 1H), 3.86-3.76 (m, 2H), 2.99-2.83 (m, 3H), 2.09-1.97 (m, 1H), 1.95-1.79 (m, 2H), 1.56-1.20 (m, 3H).

### 3-[4-[[[2-(2,6-dioxo-3-piperidyl)-1,3-dioxo-isoindolin-4-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 12)

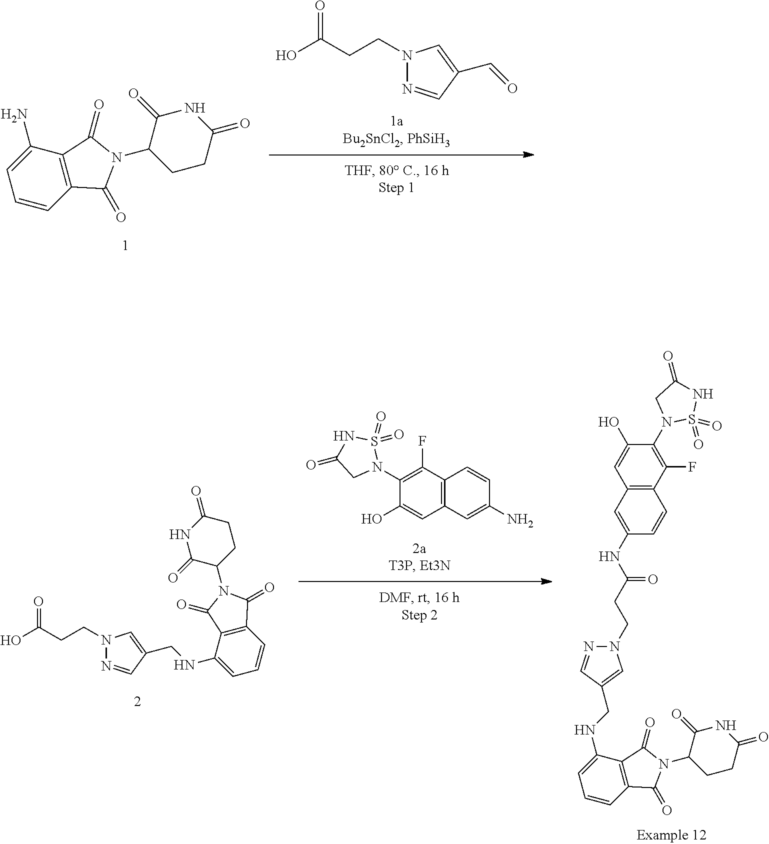 [SMILES: Nc1cccc2c1C(=O)N(C1CCC(=O)NC1=O)C2=O ; O=Cc1cnn(CCC(=O)O)c1 ; O=C(O)CCn1cc(CNc2cccc3c2C(=O)N(C2CCC(=O)NC2=O)C3=O)cn1 ; Nc1ccc2c(F)c(N3CC(=O)NS3(=O)=O)c(O)cc2c1 ; O=C1CCC(N2C(=O)c3cccc(NCc4cnn(CCC(=O)Nc5ccc6c(F)c(N7CC(=O)NS7(=O)=O)c(O)cc6c5)c4)c3C2=O)C(=O)N1]

### Step 1: 3-[4-[[[2-(2,6-dioxo-3-piperidyl)-1,3-dioxo-isoindolin-4-yl]amino]methyl]pyrazol-1-yl]propanoic acid (2)

Into a 10 mL pressure tube containing a well-stirred solution of 4-amino-2-(2,6-dioxo-3-piperidyl)isoindoline-1,3-dione (1, 150 mg, 548.96 μmol) in anhydrous THF (2 mL) were added 3-(4-formylpyrazol-1-yl)propanoic acid (1a, 138.46 mg, 823.44 μmol), dibutyltindichloride (166.80 mg, 548.96 μmol, 122.65 μL) and phenylsilane (71.29 mg, 658.75 μmol). The tube was sealed, and the reaction was stirred at 80° C. for 16 h. The volatiles were removed under reduced pressure, and the residue was purified by reverse-phase preparative HPLC [Column: Xselect C18 column (19×150) mm, 5 micron; Mobile phase A: 0.1% Formic acid in water and Mobile phase B: MeCN] to afford 3-[4-[[[2-(2,6-dioxo-3-piperidyl)-1,3-dioxo-isoindolin-4-yl]amino]methyl]pyrazol-1-yl]propanoic acid (2, 220 mg, 457.11 μmol, 83% yield, formic acid salt) as an off-white solid.

LCMS (ES+): m/z 426.0 [M+H]⁺

### Step 2: 3-[4-[[[2-(2,6-dioxo-3-piperidyl)-1,3-dioxo-isoindolin-4-yl]amino]methyl]pyrazol-1-yl]-N-[5-fluoro-7-hydroxy-6-(1,1,4-trioxo-1,2,5-thiadiazolidin-2-yl)-2-naphthyl] propanamide (Example 12)

Into a 10 mL single neck round bottom flask containing a well-stirred solution of 3-[4-[[[2-(2,6-dioxo-3-piperidyl)-1,3-dioxo-isoindolin-4-yl]amino]methyl]pyrazol-1-yl]propanoic acid (2, 40 mg, 84.85 μmol, formic acid salt) in anhydrous DMF (0.5 mL) were added 5-(6-amino-1-fluoro-3-hydroxy-2-naphthyl)-1,1-dioxo-1,2,5-thiadiazolidin-3-one (2a, 26.41 mg, 84.85 μmol), Et₃N (34.34 mg, 339.40 μmol, 47.31 μL) and 1-propanephosphonic anhydride (50 wt. % in EtOAc) (110 μL, 169.70 μmol). The reaction was stirred at ambient temperature for 16 h. The volatiles were removed under reduced pressure, and the residue was purified by reverse-phase preparative HPLC [Column: Xselect C18 column (19×150) mm, 5 micron; Mobile phase A: 0.1% TFA in water and Mobile phase B: MeCN] to afford 3-[4-[[[2-(2,6-dioxo-3-piperidyl)-1,3-dioxo-isoindolin-4-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 12, 5.5 mg, 6.30 μmol, 7% yield, TFA salt) as an off-white solid.

LCMS (ES+): m/z 719.1 [M+H]⁺

¹H NMR (400 MHZ, DMSO-d₆) δ 11.09 (s, 1H), 10.43 (s, 1H), 10.25 (s, 1H), 8.12 (s, 1H), 7.83 (d, J=8.8 Hz, 1H), 7.71 (s, 1H), 7.52-7.43 (m, 2H), 7.40-7.34 (m, 1H), 7.15-7.08 (m, 1H), 7.02-6.93 (m, 2H), 6.84 (s, 1H), 5.02 (dd, J=12.6, 5.7 Hz, 1H), 4.42-4.31 (m, 8H), 2.97-2.82 (m, 4H), 2.69-2.58 (m, 1H), 2.11-1.96 (m, 1H).

### 3-[4-[[[2-(2,6-dioxo-3-piperidyl)-1-oxo-isoindolin-4-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 13)

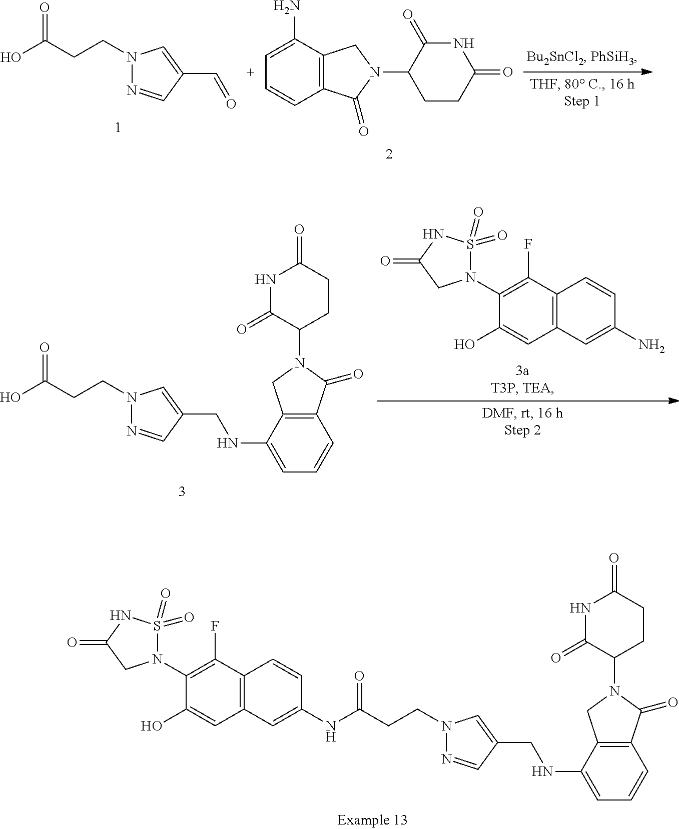

### Step 1: 3-[4-[[[2-(2,6-dioxo-3-piperidyl)-1-oxo-isoindolin-4-yl]amino]methyl]pyrazol-1-yl] propanoic acid (3)

Into a 100 mL pressure tube containing a well-stirred solution of 3-(4-formylpyrazol-1-yl)propanoic acid (1, 250 mg, 1.49 mmol) in anhydrous THF (20 mL) were added 3-(4-amino-1-oxo-isoindolin-2-yl)piperidine-2,6-dione (2, 385.46 mg, 1.49 mmol), dibutyltin dichloride (451.75 mg, 1.49 mmol, 332.17 μL) and phenylsilane (193.07 mg, 1.78 mmol). The vial was sealed, and the suspension was stirred at 80° C. for 16 h. The solvent was removed under reduced pressure and the residue purified by silica gel chromatography (0-5% MeOH in DCM) to afford 3-[4-[[[2-(2,6-dioxo-3-piperidyl)-1-oxo-isoindolin-4-yl]amino]methyl]pyrazol-1-yl]propanoic acid (3, 230 mg, 409.79 μmol, 28% yield) as an off-white solid.

LCMS (ES−): m/z 410.2 [M−H]⁻

### Step 2: 3-[4-[[[2-(2,6-dioxo-3-piperidyl)-1-oxo-isoindolin-4-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 13)

Into a 10 mL single neck round bottom flask containing a well-stirred solution of 3-[4-[[[2-(2,6-dioxo-3-piperidyl)-1-oxo-isoindolin-4-yl]amino]methyl]pyrazol-1-yl]propanoic acid (3, 35 mg, 85.07 μmol) in anhydrous DMF (1 mL) were added 5-(6-amino-1-fluoro-3-hydroxy-2-naphthyl)-1,1-dioxo-1,2,5-thiadiazolidin-3-one (3a, 26.48 mg, 85.07 μmol), triethylamine (25.83 mg, 255.22 μmol, 35.57 μL) and 1-propanephosphonic anhydride (50% in EtOAc) (40.60 mg, 127.61 μmol, 90 μL). After 16 h, the solvent was evaporated under reduced pressure and the residue purified by reverse phase prep HPLC [Purification method: Column: X select (150×19)mm, 5 μm; Mobile phase A: 0.1% TFA in MQ-water; Mobile phase B: Acetonitrile] to afford 3-[4-[[[2-(2,6-dioxo-3-piperidyl)-1-oxo-isoindolin-4-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 13, 5.5 mg, 6.58 μmol, 8% yield, TFA Salt) as an off-white solid.

LCMS (ES+): m/z 705.4 [M+H]⁺

¹H NMR (400 MHZ, DMSO-d₆) δ 11.00 (s, 1H), 10.42-10.14 (m, 2H), 8.13 (s, 1H), 7.83 (d, J=9.0 Hz, 1H), 7.68 (s, 1H), 7.43 (s, 1H), 7.38 (dd, J=9.1, 1.9 Hz, 1H), 7.21 (t, J=7.7 Hz, 1H), 6.97-6.89 (m, 2H), 6.78 (d, J=8.1 Hz, 1H), 5.09 (dd, J=13.3, 5.1 Hz, 1H), 4.37 (t, J=6.7 Hz, 2H), 4.31-4.25 (m, 2H), 4.23-4.10 (m, 4H), 2.97-2.86 (m, 3H), 2.64-2.57 (m, 2H), 2.33-2.22 (m, 1H), 2.06-1.95 (m, 1H).

### N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1,3-dioxoisoindolin-5-yl)amino)methyl)-1H-1,2,3-triazol-1-yl)propanamide (Example 14)

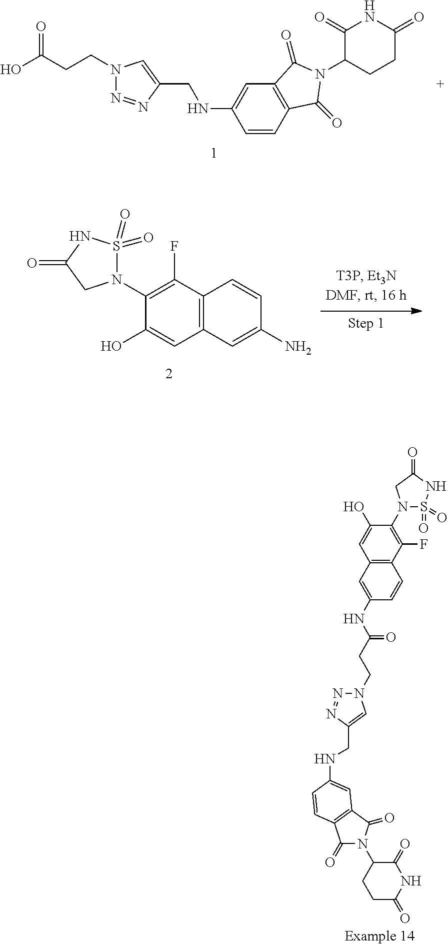 [SMILES: O=C(O)CCn1cc(CNc2ccc3c(c2)C(=O)N(C2CCC(=O)NC2=O)C3=O)nn1 ; Nc1ccc2c(F)c(N3CC(=O)NS3(=O)=O)c(O)cc2c1 ; O=C1CCC(N2C(=O)c3ccc(NCc4cn(CCC(=O)Nc5ccc6c(F)c(N7CC(=O)NS7(=O)=O)c(O)cc6c5)nn4)cc3C2=O)C(=O)N1]

### Step 1: N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1,3-dioxoisoindolin-5-yl)amino)methyl)-1H-1,2,3-triazol-1-yl)propanamide (Example 14)

Into a 10 mL three neck round bottom flask containing a well-stirred solution of 3-[4-[[[2-(2,6-dioxo-3-piperidyl)-1,3-dioxo-isoindolin-5-yl]amino]methyl]triazol-1-yl]propanoic acid (1, 30 mg, 55.51 μmol, TFA salt) in anhydrous DMF (1 mL) were added Et₃N (16.85 mg, 166.54 μmol, 23.21 μL), 1-propanephosphonic anhydride (50% in EtOAc) (90 μL, 138.78 μmol) and 5-(6-amino-1-fluoro-3-hydroxy-2-naphthyl)-1,1-dioxo-1,2,5-thiadiazolidin-3-one (2, 19.30 mg, 55.51 μmol, HCl salt) in anhydrous DMF (0.5 mL). The reaction was stirred at ambient temperature for 16 h. The reaction mixture was concentrated under reduced pressure, and the residue purified by reverse-phase preparative HPLC [Column: X BRIDGE C18 column (19×150) mm 5 micron; Mobile phase A: 0.1% TFA in water and Mobile phase B: MeCN] to afford N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1,3-dioxoisoindolin-5-yl)amino)methyl)-1H-1,2,3-triazol-1-yl)propanamide (Example 14, 5.2 mg, 5.61 μmol, 10% yield, TFA salt) as a yellow solid.

LCMS (ES+): m/z 720.0 [M+H]⁺

¹H NMR (400 MHZ, DMSO-d₆) δ 11.06 (s, 1H), 10.28 (s, 1H), 9.90 (s, 1H), 8.09 (s, 1H), 8.03 (s, 1H), 7.81 (d, J=9.0 Hz, 1H), 7.57-7.53 (m, 2H), 7.35 (d, J=9.0 Hz, 1H), 7.05 (s, 1H), 6.96-6.90 (m, 2H), 5.02 (dd, J=12.7, 5.4 Hz, 1H), 4.65 (dd, J=6.7 Hz, 2H), 4.45 (d, J=3.9 Hz, 2H), 4.10 (s, 2H), 3.05-2.98 (m, 3H), 2.93-2.80 (m, 3H), 2.03-1.94 (m, 1H).

### 3-[4-[[[2-(2,6-dioxo-3-piperidyl)-1-oxo-isoindolin-5-yl]amino]methyl]triazol-1-yl]-N-[5-fluoro-7-hydroxy-6-(1,1,4-trioxo-1,2,5-thiadiazolidin-2-yl)-2-naphthyl]propanamide

### Example 15

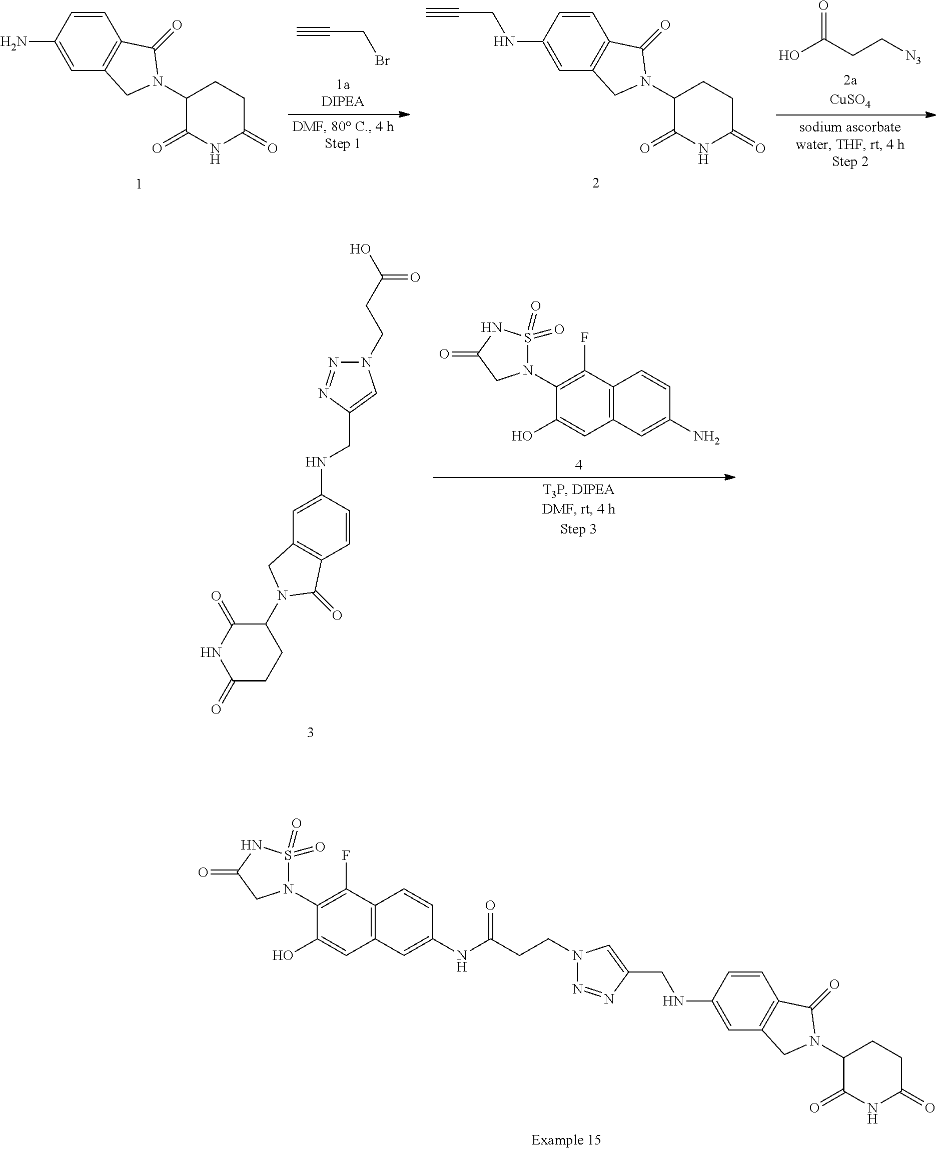

### Step 1: 3-[1-oxo-5-(prop-2-ynylamino)isoindolin-2-yl]piperidine-2,6-dione (2)

Into a 25 mL single neck round bottom flask containing a well-stirred solution of 3-(5-amino-1-oxo-isoindolin-2-yl)piperidine-2,6-dione (1, 0.200 g, 771.43 μmol) in anhydrous DMF (5 mL) was added DIPEA (199.40 mg, 1.54 mmol, 268.74 μL) and 3-bromoprop-1-yne (1a, 91.77 mg, 771.43 μmol). The mixture was stirred at 80° C. for 4 h. The volatiles were removed under reduced pressure and the residue purified by silica gel chromatography (30-40% EtOAc in petroleum ether) to afford 3-[1-oxo-5-(prop-2-ynylamino)isoindolin-2-yl]piperidine-2,6-dione (2, 0.110 g, 351.49 μmol, 46% yield) as a yellow solid.

LCMS (ES+): m/z 298.1 [M+H]⁺

### Step 2: 3-[4-[[[2-(2,6-dioxo-3-piperidyl)-1-oxo-isoindolin-5-yl]amino]methyl]triazol-1-yl]propanoic acid (3)

Into a 25 mL single neck round bottom flask containing a well-stirred solution of 3-azidopropanoic acid (2a, 38.71 mg, 336.35 μmol) and 3-[1-oxo-5-(prop-2-ynylamino)isoindolin-2-yl]piperidine-2,6-dione (2, 0.100 g, 336.35 μmol) in a mixture of THF (5 mL) and water (5 mL) was added sodium ascorbate (66.63 mg, 336.35 μmol) followed by copper sulphate (53.69 mg, 336.35 μmol, 14.91 μL). After 4 h the organic layer was separated and solvent removed. The residue was purified by preparatory HPLC Method [Column: Biotage snap Ultra C18 (30 g) (19×150 mm), 25 μm, Mobile phase A: 0.1% Ammonium Acetate in H₂O, Mobile phase B: ACN; Flow Rate: 15.0 mL/min]. Product-containing fractions were combined, the solvent removed and the solid dissolved in EtOAc (10 mL) and washed with water (15 mL). The organic layer was dried over sodium sulphate, filtered and solvent removed to afford 3-[4-[[[2-(2,6-dioxo-3-piperidyl)-1-oxo-isoindolin-5-yl]amino]methyl]triazol-1-yl]propanoic acid (3, 0.080 g, 188.17 μmol, 56% yield) as a yellow solid.

LCMS (ES+): m/z 413.2 [M+H]⁺

### Step 3: 3-[4-[[[2-(2,6-dioxo-3-piperidyl)-1-oxo-isoindolin-5-yl]amino]methyl]triazol-1-yl]-N-[5-fluoro-7-hydroxy-6-(1,1,4-trioxo-1,2,5-thiadiazolidin-2-yl)-2-naphthyl] propanamide (Example 15)

Into a 50 mL round-bottomed flask containing a well-stirred solution of 3-[4-[[[2-(2,6-dioxo-3-piperidyl)-1-oxo-isoindolin-5-yl]amino]methyl]triazol-1-yl]propanoic acid (3, 13.25 mg, 32.12 μmol) in dry DMF (0.2 mL) were added DIPEA (8.30 mg, 64.25 μmol, 11.19 μL) and T₃P (50 wt % in EtOAc, 20.4 μL, 10.22 mg, 32.12 μmol). After 10 min, 5-(6-amino-1-fluoro-3-hydroxy-2-naphthyl)-1,1-dioxo-1,2,5-thiadiazolidin-3-one (4, 10 mg, 32.12 μmol) was added. After 4 h the volatiles were removed under reduced pressure. The residue was purified by reverse phase prep HPLC [Purification method: Column: X-Bridge C18 (19×150 mm) 5 μm, Mobile phase A: 0.1% ammonium acetate in H₂O, Mobile phase B: ACN; Flow Rate: 15.0 mL/min] to afford 3-[4-[[[2-(2,6-dioxo-3-piperidyl)-1-oxo-isoindolin-5-yl]amino]methyl]triazol-1-yl]-N-[5-fluoro-7-hydroxy-6-(1,1,4-trioxo-1,2,5-thiadiazolidin-2-yl)-2-naphthyl]propanamide (Example 15, 2.3 mg, 3.03 μmol, 9% yield) as a yellow solid.

LCMS (ES+): m/z 706.2 [M+H]⁺

¹H NMR (400 MHZ, DMSO-d₆) δ 10.93 (s, 1H), 10.26 (s, 1H), 9.74 (s, 1H), 8.10 (s, 1H), 8.00 (s, 1H), 7.81 (d, J=8.9 Hz, 1H), 7.41-7.34 (m, 2H), 7.08 (s, 4H), 6.93 (s, 1H), 6.88-6.82 (m, 1H), 6.75-6.70 (m, 2H), 5.00 (dd, J=13.4, 5.1 Hz, 1H), 4.65 (t, J=6.8 Hz, 2H), 4.36 (d, J=5.7 Hz, 2H), 4.25 (d, J=16.7 Hz, 1H), 4.12 (d, J=16.7 Hz, 1H), 4.06 (s, 2H), 3.02 (t, J=6.7 Hz, 2H), 2.94-2.82 (m, 4H), 1.97-1.87 (m, 1H).

### 3-[4-[[[2-(2,6-dioxo-3-piperidyl)-1-oxo-isoindolin-5-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 16)

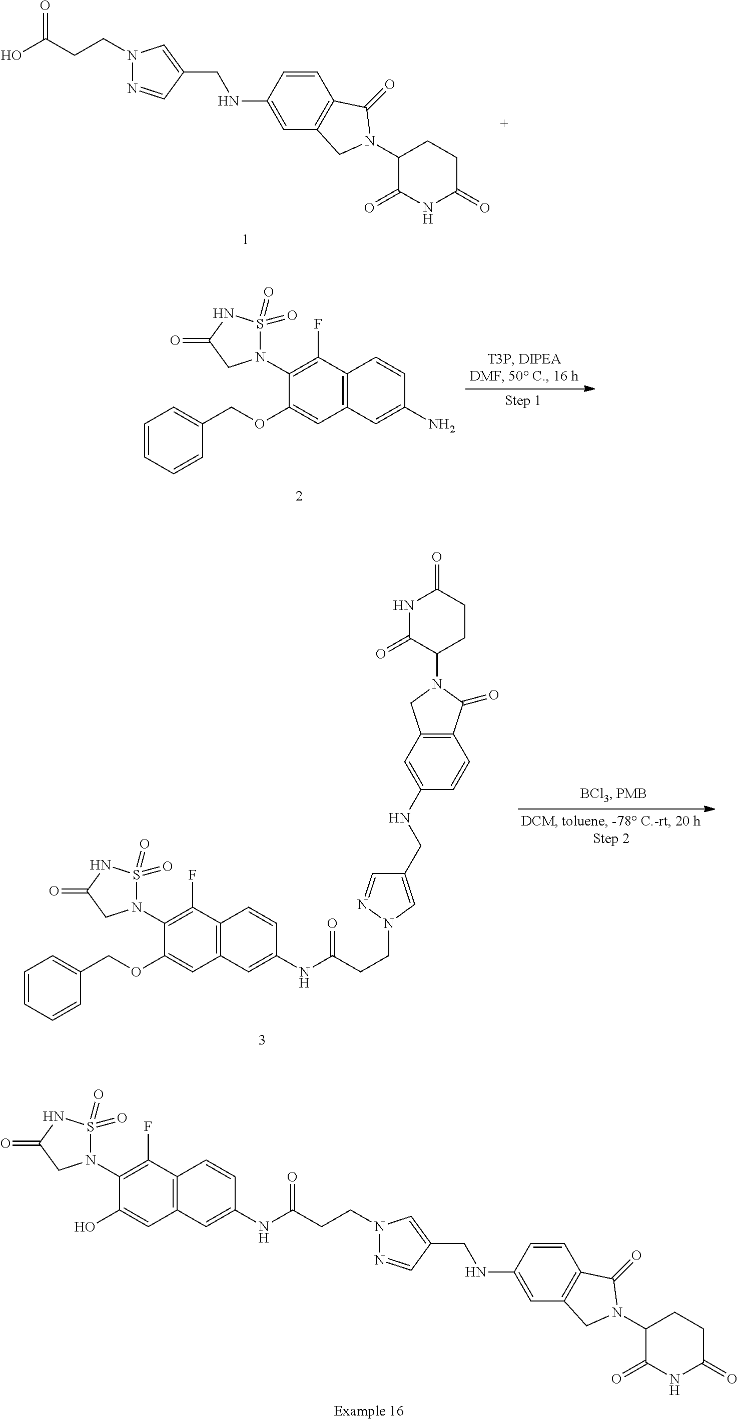 [SMILES: O=C(O)CCn1cc(CNc2ccc3c(c2)CN(C2CCC(=O)NC2=O)C3=O)cn1 ; Nc1ccc2c(F)c(N3CC(=O)NS3(=O)=O)c(OCc3ccccc3)cc2c1 ; O=C1CCC(N2Cc3cc(NCc4cnn(CCC(=O)Nc5ccc6c(F)c(N7CC(=O)NS7(=O)=O)c(OCc7ccccc7)cc6c5)c4)ccc3C2=O)C(=O)N1 ; O=C1CCC(N2Cc3cc(NCc4cnn(CCC(=O)Nc5ccc6c(F)c(N7CC(=O)NS7(=O)=O)c(O)cc6c5)c4)ccc3C2=O)C(=O)N1]

### Step 1: N-[7-benzyloxy-5-fluoro-6-(1,1,4-trioxo-1,2,5-thiadiazolidin-2-yl)-2-naphthyl]-3-[4-[[[2-(2,6-dioxo-3-piperidyl)-1-oxo-isoindolin-5-yl]amino]methyl]pyrazol-1-yl] propanamide (3)

Into a 10 mL single neck round bottom flask containing a well-stirred solution of 3-[4-[[[2-(2,6-dioxo-3-piperidyl)-1-oxo-isoindolin-5-yl]amino]methyl]pyrazol-1-yl]propanoic acid (1, 139.63 mg, 247.41 μmol, TFA salt) in DMF (2 mL) were added DIPEA (159.88 mg, 1.24 mmol, 215.48 μL) and 1-propanephosphonic anhydride (50 wt. % in EtOAc) (0.19 mL, 94.47 mg, 296.90 μmol) and the reaction mixture was stirred at RT for 10 min. Then a solution of 5-(6-amino-3-benzyloxy-1-fluoro-2-naphthyl)-1,1-dioxo-1,2,5-thiadiazolidin-3-one (2, 127.53 mg, 247.41 μmol, TFA salt) in DMF (0.5 mL) and DIPEA (0.5 mL) were added dropwise and the reaction mixture was heated at 50° C. for 16 h. The solvent was removed under reduced pressure. The residue was purified by reverse phase preparative HPLC [Purification method: Column: Sunfire C18, (19×150 mm), 5 μm, Mobile phase A: 0.1 TFA in water and Mobile phase B: MeCN] to afford N-[7-benzyloxy-5-fluoro-6-(1,1,4-trioxo-1,2,5-thiadiazolidin-2-yl)-2-naphthyl]-3-[4-[[[2-(2,6-dioxo-3-piperidyl)-1-oxo-isoindolin-5-yl]amino]methyl]pyrazol-1-yl]propanamide (3, 90 mg, 65.75 μmol, 27% yield, TFA salt) as an off-white solid. LCMS (ES+): m/z 795.1 [M+H]⁺

### Step 2: 3-[4-[[[2-(2,6-dioxo-3-piperidyl)-1-oxo-isoindolin-5-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 16)

Into a 10 mL single neck round bottom flask containing a well-stirred solution of N-[7-benzyloxy-5-fluoro-6-(1,1,4-trioxo-1,2,5-thiadiazolidin-2-yl)-2-naphthyl]-3-[4-[[[2-(2,6-dioxo-3-piperidyl)-1-oxo-isoindolin-5-yl]amino]methyl]pyrazol-1-yl]propanamide (3, 35 mg, 25.57 μmol, TFA salt) in a mixture of toluene (0.5 mL) and DCM (0.5 mL) was added pentamethylbenzene (11.37 mg, 76.71 μmol) and the reaction mixture was cooled to −78° C. Then BCl₃ (1 M in DCM) (4 mmol, 4 mL) was added dropwise over a period of 2 min and the reaction mixture was stirred at RT for 20 h. The reaction mixture was cooled to −78° C. and quenched with 10% DCM in MeOH (4 mL), brought to RT and concentrated under reduced pressure at 35° C. The residue was purified by reverse phase preparative HPLC [Purification method: Column: X-Bridge C18, (150×19 mm, 5 μm, Mobile phase A-0.1% TFA in water and Mobile phase B-MeCN] to yield 3-[4-[[[2-(2,6-dioxo-3-piperidyl)-1-oxo-isoindolin-5-yl]amino]methyl]pyrazol-1-yl]-N-[5-fluoro-7-hydroxy-6-(1,1,4-trioxo-1,2,5-thiadiazolidin-2-yl)-2-naphthyl]propanamide (Example 16, 2.75 mg, 3.30 μmol, 13% yield, TFA salt) as an off-white solid.

LCMS (ES+): m/z 705.3 [M+H]⁺

¹H NMR (400 MHZ, DMSO-d₆) δ 10.92 (s, 1H), 10.24 (s, 1H), 10.13 (s, 1H), 8.14 (s, 1H), 7.83 (d, J=9.0 Hz, 1H), 7.70 (s, 1H), 7.43 (s, 1H), 7.41-7.35 (m, 2H), 6.95 (s, 1H), 6.73-6.66 (m, 2H), 5.00 (dd, J=13.3, 5.1 Hz, 1H), 4.39 (t, J=6.7 Hz, 2H), 4.29-4.18 (m, 3H), 4.14 (s, 2H), 3.15-3.05 (m, 1H), 2.93 (t, J=6.7 Hz, 2H), 2.91-2.80 (m, 1H), 2.60-2.55 (m, 1H), 2.37-2.23 (m, 1H), 1.98-1.88 (m, 1H).

### 3-[4-[[[2-(2,6-dioxo-3-piperidyl)-1,3-dioxo-isoindolin-4-yl]amino]methyl]triazol-1-yl]-N-[5-fluoro-7-hydroxy-6-(1,1,4-trioxo-1,2,5-thiadiazolidin-2-yl)-2-naphthyl]azetidine-1-carboxamide (Example 17)

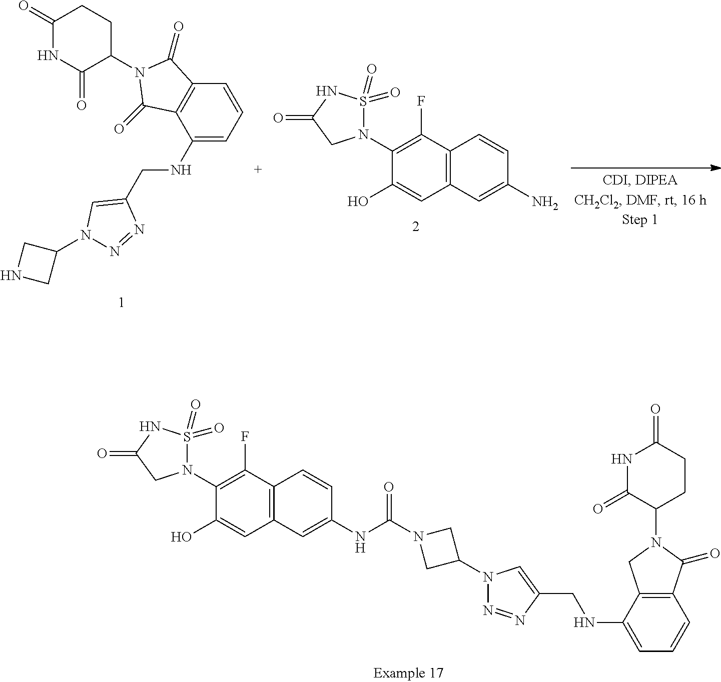

### Step 1: 3-[4-[[[2-(2,6-dioxo-3-piperidyl)-1,3-dioxo-isoindolin-4-yl]amino]methyl]triazol-1-yl]-N-[5-fluoro-7-hydroxy-6-(1,1,4-trioxo-1,2,5-thiadiazolidin-2-yl)-2-naphthyl] azetidine-1-carboxamide (Example 17)

Into a 50 mL single neck round bottom flask containing a well-stirred solution of 5-(6-amino-1-fluoro-3-hydroxy-2-naphthyl)-1,1-dioxo-1,2,5-thiadiazolidin-3-one (2, 30 mg, 70.54 μmol, TFA salt) in DCM (1 mL) were added DIPEA (742.00 mg, 5.74 mmol, 1.0 mL) and CDI (57.19 mg, 352.68 μmol) and the resulting reaction mixture was stirred at for 3 h. To this, 4-[[1-(azetidin-3-yl)triazol-4-yl]methylamino]-2-(2,6-dioxo-3-piperidyl) isoindoline-1,3-dione (1, 28.88 mg, 70.54 μmol) was added. After 16 h the reaction mixture was concentrated under reduced pressure and the residue was purified by reverse-phase preparative HPLC [Column: PFP (pentafluoro phenyl)-YMC (20×250 mm) 5 micron; Mobile phase A: 0.1% TFA in water and Mobile phase B: MeCN] to obtain 3-[4-[[[2-(2,6-dioxo-3-piperidyl)-1,3-dioxo-isoindolin-4-yl]amino]methyl]triazol-1-yl]-N-[5-fluoro-7-hydroxy-6-(1,1,4-trioxo-1,2,5-thiadiazolidin-2-yl)-2-naphthyl]azetidine-1-carboxamide (Example 17, 30 mg, 31.71 μmol, 44.95% yield, TFA salt) as a yellow solid.

LCMS (ES+): m/z 747.2 [M+H]⁺

¹H NMR (400 MHZ, DMSO-d₆) δ 11.10 (s, 1H), 9.84 (s, 1H), 8.88 (s, 1H), 8.32 (d, J=3.3 Hz, 1H), 7.98-7.91 (m, 1H), 7.82-7.76 (m, 1H), 7.62-7.54 (m, 1H), 7.52-7.45 (m, 1H), 7.24-7.16 (m, 1H), 7.14-7.04 (m, 2H), 5.56-5.44 (m, 1H), 5.10-5.01 (m, 1H), 4.68-4.61 (m, 2H), 4.57-4.47 (m, 2H), 4.35-4.24 (m, 2H), 4.16 (s, 2H), 2.95-2.81 (m, 2H), 2.71-2.58 (m, 2H), 2.07-1.97 (m, 1H).

### N-(6-(1,1-dioxido-4-oxo-1,2,5-thiadiazolidin-2-yl)-5-fluoro-7-hydroxynaphthalen-2-yl)-3-(4-(2-((2-(2,6-dioxopiperidin-3-yl)-1,3-dioxoisoindolin-4-yl)amino)propan-2-yl)-1H-1,2,3-triazol-1-yl)propanamide (Example 18)

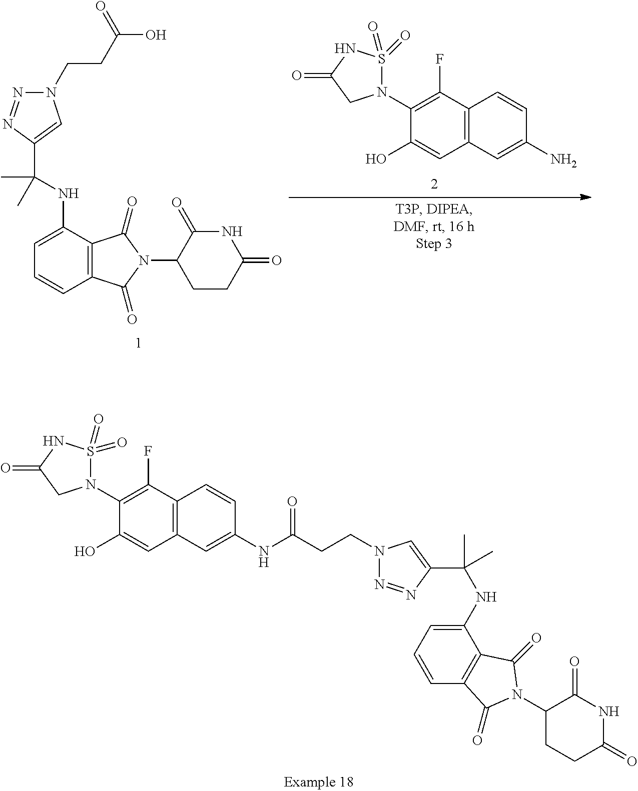 [SMILES: CC(C)(Nc1cccc2c1C(=O)N(C1CCC(=O)NC1=O)C2=O)c1cn(CCC(=O)O)nn1 ; Nc1ccc2c(F)c(N3CC(=O)NS3(=O)=O)c(O)cc2c1 ; CC(C)(Nc1cccc2c1C(=O)N(C1CCC(=O)NC1=O)C2=O)c1cn(CCC(=O)Nc2ccc3c(F)c(N4CC(=O)NS4(=O)=O)c(O)cc3c2)nn1]

### Step 1: N-(6-(1,1-dioxido-4-oxo-1,2,5-thiadiazolidin-2-yl)-5-fluoro-7-hydroxynaphthalen-2-yl)-3-(4-(2-((2-(2,6-dioxopiperidin-3-yl)-1,3-dioxoisoindolin-4-yl)amino)propan-2-yl)-1H-1,2,3-triazol-1-yl)propanamide (Example 18)

Into a 20 mL capped vial containing a well-stirred solution of 3-[4-[1-[[2-(2,6-dioxo-3-piperidyl)-1,3-dioxo-isoindolin-4-yl]amino]-1-methyl-ethyl]triazol-1-yl]propanoic acid (5, 0.050 g, 110.03 μmol) in DMF (1 mL) were added 5-(6-amino-1-fluoro-3-hydroxy-2-naphthyl)-1,1-dioxo-1,2,5-thiadiazolidin-3-one (6, 34.25 mg, 110.03 μmol) and N,N-diisopropylethylamine (71.10 mg, 550.13 μmol, 95.82 μL) and the mixture was stirred at RT for 5 min. 1-propanephosphonic anhydride solution (50% in ethyl acetate) (0.077 mL, 38.51 mg, 121.03 μmol) was added slowly and the resulting mixture was stirred at RT for 16 h. The solvent was removed under reduced pressure, and the residue was purified by reverse phase prep HPLC [Purification method: Column: X-bridge, C18 (150×19) mm, 5 micron; Mobile phase A: 0.1% HCOOH in water and Mobile phase B: MeCN] to yield N-(6-(1,1-dioxido-4-oxo-1,2,5-thiadiazolidin-2-yl)-5-fluoro-7-hydroxynaphthalen-2-yl)-3-(4-(2-((2-(2,6-dioxopiperidin-3-yl)-1,3-dioxoisoindolin-4-yl)amino)propan-2-yl)-1H-1,2,3-triazol-1-yl)propanamide (Example 18, 7 mg, 8.40 μmol, 8% yield, formate salt) as a yellow solid. LCMS (ES−): m/z 746.4 [M−H]^{- 1}H NMR (400 MHZ, DMSO-d₆) δ 11.11 (s, 1H), 10.35-10.21 (m, 2H), 8.11-8.07 (m, 2H), 7.83 (d, J=9.0 Hz, 1H), 7.35 (dd, J=9.1, 1.9 Hz, 1H), 7.25-7.18 (m, 1H), 6.97-6.93 (m, 1H), 6.90 (d, J=7.1 Hz, 1H), 6.87 (s, 1H), 6.56 (d, J=8.6 Hz, 1H), 5.05 (dd, J=12.8, 5.4 Hz, 1H), 4.64 (t, J=6.7 Hz, 2H), 4.33 (s, 2H), 3.01 (t, J=6.8 Hz, 2H), 2.94-2.80 (m, 1H), 2.62-2.54 (m, 2H), 2.07-1.98 (m, 1H), 1.70 (d, J=1.9 Hz, 6H).

### N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1-oxoisoindolin-4-yl)oxy)methyl)-1H-1,2,3-triazol-1-yl) propanamide (Example 19)

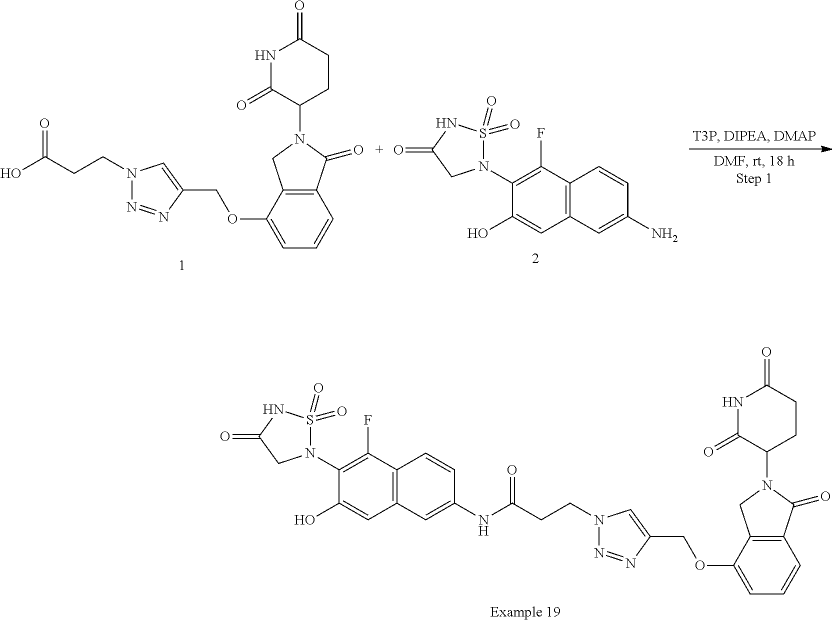

### Step 1: N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1-oxoisoindolin-4-yl)oxy)methyl)-1H-1,2,3-triazol-1-yl)propanamide (Example 19)

Into a 20 mL capped vial containing a well-stirred solution of 3-[4-[[2-(2,6-dioxo-3-piperidyl)-1-oxo-isoindolin-4-yl]oxymethyl]triazol-1-yl]propanoic acid (6, 60 mg, 113.76 μmol, TFA salt) in DMF (1 mL) was added DIPEA (93.79 mg, 725.72 μmol, 126.40 μL) followed by 1-propanephosphonic anhydride (50% in EtOAc) (138.5 μL, 69.27 mg, 217.72 μmol,). After 5 min 5-(6-amino-1-fluoro-3-hydroxy-2-naphthyl)-1,1-dioxo-1,2,5-thiadiazolidin-3-one (7, 27.11 mg, 87.09 μmol) in DMF (0.3 mL) was added and the reaction mixture stirred for 18 h. The reaction mixture was concentrated under reduced pressure and purified by reverse phase preparative HPLC [Purification method: Column: X-Select C18 (150×19 mm), 5 μm; Mobile phase A: 0.1% TFA in water and Mobile phase B: MeCN] to afford N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1-oxoisoindolin-4-yl)oxy)methyl)-1H-1,2,3-triazol-1-yl)propanamide (Example 19, 8.8 mg, 10.24 μmol, 9% yield, TFA salt) as an-off white solid. LCMS (ES+): m/z 707.0 [M+H]^{+ 1}H NMR (400 MHz, DMSO-d₆) δ 10.96 (s, 1H), 10.31 (s, 1H), 10.25 (s, 1H), 8.28 (s, 1H), 8.13 (s, 1H), 7.84 (d, J=9.0 Hz, 1H), 7.50-7.42 (m, 2H), 7.39 (dd, J=9.1, 2.0 Hz, 1H), 7.34-7.29 (m, 1H), 6.95 (d, J=2.7 Hz, 1H), 5.29 (s, 2H), 5.09 (dd, J=13.4, 5.1 Hz, 1H), 4.69 (t, J=6.6 Hz, 2H), 4.38-4.29 (m, 3H), 4.18 (d, J=17.5 Hz, 1H), 3.07 (t, J=6.7 Hz, 2H), 2.89 (ddd, J=18.0, 13.6, 5.4 Hz, 2H), 2.45-2.36 (m, 2H), 2.02-1.90 (m, 1H).

### 3-[4-[[2-(2,6-dioxo-3-piperidyl)-1-oxo-isoindolin-5-yl]oxymethyl]triazol-1-yl]-N-[5-fluoro-7-hydroxy-6-(1,1,4-trioxo-1,2,5-thiadiazolidin-2-yl)-2-naphthyl]propanamide (Example 20)

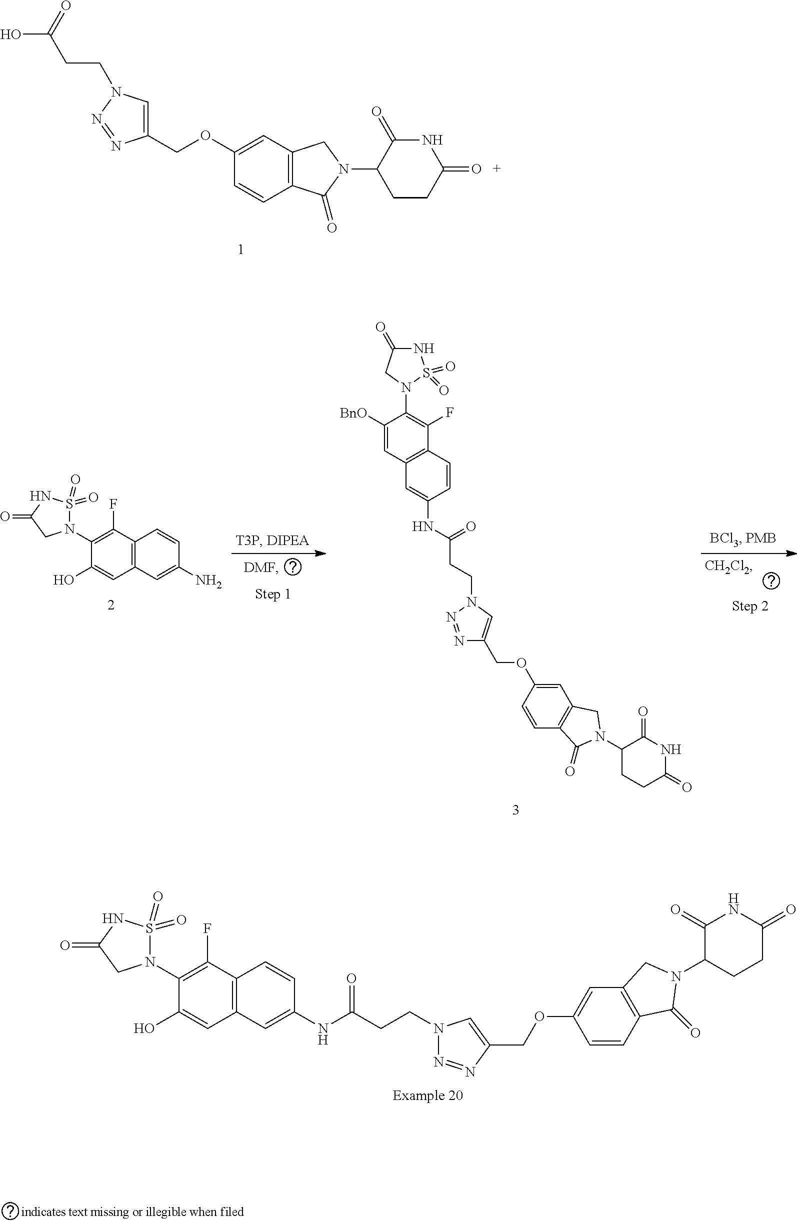 [SMILES: O=C(O)CCn1cc(COc2ccc3c(c2)CN(C2CCC(=O)NC2=O)C3=O)nn1 ; Nc1ccc2c(F)c(N3CC(=O)NS3(=O)=O)c(O)cc2c1 ; O=C1CCC(N2Cc3cc(OCc4cn(CCC(=O)Nc5ccc6c(F)c(N7CC(=O)NS7(=O)=O)c(OCc7ccccc7)cc6c5)nn4)ccc3C2=O)C(=O)N1 ; O=C1CCC(N2Cc3cc(OCc4cn(CCC(=O)Nc5ccc6c(F)c(N7CC(=O)NS7(=O)=O)c(O)cc6c5)nn4)ccc3C2=O)C(=O)N1]

### Step 1: N-[7-benzyloxy-5-fluoro-6-(1,1,4-trioxo-1,2,5-thiadiazolidin-2-yl)-2-naphthyl]-3-[4-[[2-(2,6-dioxo-3-piperidyl)-1-oxo-isoindolin-5-yl]oxymethyl]triazol-1-yl]propanamide (3)

Into a 10 mL single neck round bottom flask containing a well-stirred solution of 5-(6-amino-3-benzyloxy-1-fluoro-2-naphthyl)-1,1-dioxo-1,2,5-thiadiazolidin-3-one (2, 24.28 mg, 60.48 μmol) and 3-[4-[[2-(2,6-dioxo-3-piperidyl)-1-oxo-isoindolin-5-yl]oxymethyl]triazol-1-yl]propanoic acid (1, 25 mg, 60.48 μmol) in anhydrous DMF (0.25 mL) was added DIPEA (23.45 mg, 181.43 μmol, 31.60 μL). Propylphosphonic anhydride (50 wt. % in EtOAc) (57.72 μL, 28.86 mg, 90.71 μmol) was added to the reaction at 0° C. and the resulting mixture was stirred at RT for 16 h. The reaction mixture was concentrated under reduced pressure and the residue purified by reverse-phase preparatory HPLC [X-BRIDGE C18 (19×150) mm, 5.0 μm with Solvent A: 0.1% TFA in water; Solvent B: Acetonitrile] to afford N-[7-benzyloxy-5-fluoro-6-(1,1,4-trioxo-1,2,5-thiadiazolidin-2-yl)-2-naphthyl]-3-[4-[[2-(2,6-dioxo-3-piperidyl)-1-oxo-isoindolin-5-yl] oxymethyl]triazol-1-yl]propanamide (3, 20 mg, 21.60 μmol, 36% yield, TFA salt) as an off-white powder. LCMS (ESI+): m/z 797.6 [M+H]⁺

### Step 2: 3-[4-[[2-(2,6-dioxo-3-piperidyl)-1-oxo-isoindolin-5-yl]oxymethyl]triazol-1-yl]-N-[5-fluoro-7-hydroxy-6-(1,1,4-trioxo-1,2,5-thiadiazolidin-2-yl)-2-naphthyl]propanamide (Example 20)

Into a 10 mL single neck round bottom flask containing a well-stirred solution of N-[7-benzyloxy-5-fluoro-6-(1,1,4-trioxo-1,2,5-thiadiazolidin-2-yl)-2-naphthyl]-3-[4-[[2-(2,6-dioxo-3-piperidyl)-1-oxo-isoindolin-5-yl]oxymethyl]triazol-1-yl]propanamide (3, 20 mg, 25.10 μmol) in anhydrous CH₂Cl₂ (1.2 mL) and anhydrous toluene (1.2 mL) was added pentamethylbenzene (5.58 mg, 37.65μ, 6.09 μL). The reaction mixture was cooled to −78° C. and BCl₃ (1 M in DCM) (14.71 mg, 125.51 μmol, 2 mL) was added. The resulting solution was stirred at RT for 36 h. The reaction mixture was cooled to −78° C. and quenched with 5% MeOH in DCM (5 mL). The reaction mixture was concentrated under reduced pressure and purified by reverse-phase preparatory HPLC [Purification method: Column: X-BRIDGE C18 (19×150) mm, 5.0 μm; Mobile phase A: 0.1% TFA in water; Mobile phase B: Acetonitrile] to afford 3-[4-[[2-(2,6-dioxo-3-piperidyl)-1-oxo-isoindolin-5-yl]oxymethyl]triazol-1-yl]-N-[5-fluoro-7-hydroxy-6-(1,1,4-trioxo-1,2,5-thiadiazolidin-2-yl)-2-naphthyl]propanamide (Example 20, 8 mg, 9.68 μmol, 39% yield, TFA salt) as an off-white solid. LCMS (ESI+): m/z 707.0 [M+H]^{+ 1}H NMR (400 MHZ, DMSO-d₆) δ 10.97 (s, 1H), 10.31 (s, 1H), 9.93 (s, 1H), 8.26 (s, 1H), 8.09 (s, 1H), 7.81 (d, J=9.0 Hz, 1H), 7.63 (d, J=8.4 Hz, 1H), 7.36 (dd, J=9.0, 1.9 Hz, 1H), 7.29-7.25 (m, 1H), 7.12 (dd, J=8.3, 2.3 Hz, 1H), 6.93 (s, 1H), 5.22 (s, 2H), 5.04 (dd, J=13.3, 5.2 Hz, 1H), 4.69 (t, J=6.6 Hz, 2H), 4.38 (d, J=17.3 Hz, 1H), 4.25 (d, J=17.2 Hz, 1H), 4.07 (s, 2H), 3.05 (t, J=6.5 Hz, 2H), 2.95-2.82 (m, 1H), 2.61 (d, J=3.0 Hz, 2H), 2.42-2.30 (m, 1H), 2.02-1.93 (m, 1H).

### N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1-oxoisoindolin-4-yl)oxy)methyl)-1H-pyrazol-1-yl)propanamide (Example 21)

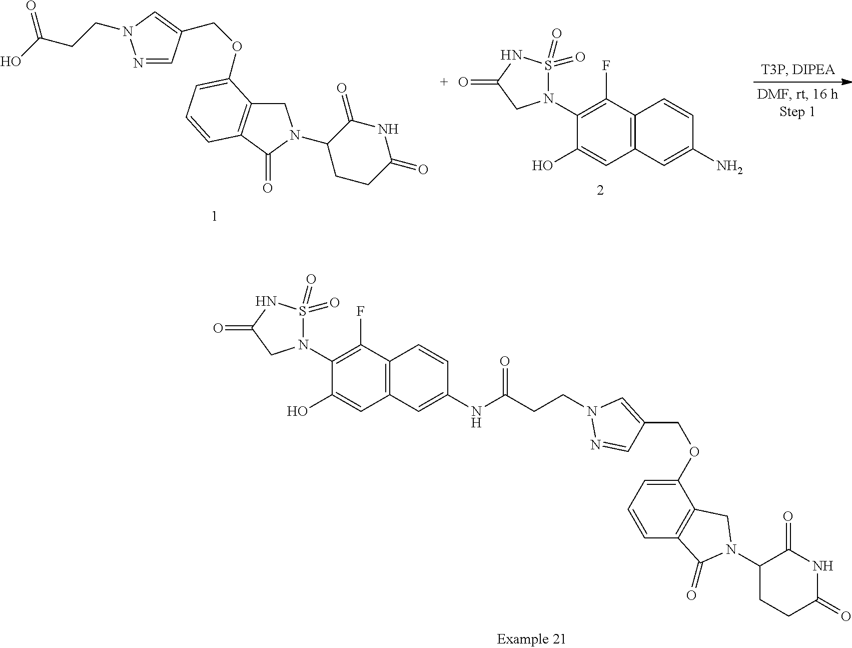 [SMILES: O=C(O)CCn1cc(COc2cccc3c2CN(C2CCC(=O)NC2=O)C3=O)cn1 ; Nc1ccc2c(F)c(N3CC(=O)NS3(=O)=O)c(O)cc2c1 ; O=C1CCC(N2Cc3c(OCc4cnn(CCC(=O)Nc5ccc6c(F)c(N7CC(=O)NS7(=O)=O)c(O)cc6c5)c4)cccc3C2=O)C(=O)N1]

### Step 1: N-(6-(1,1-dioxido-4-oxo-1,2,5-thiadiazolidin-2-yl)-5-fluoro-7-hydroxynaphthalen-2-yl)-3-(4-(((2-(2,6-dioxopiperidin-3-yl)-1-oxoisoindolin-4-yl)oxy)methyl)-1H-pyrazol-1-yl)propanamide (Example 21)

Into a 10 mL single neck round bottom flask containing a well-stirred solution of 3-[4-[[2-(2,6-dioxo-3-piperidyl)-1-oxo-isoindolin-4-yl]oxymethyl]pyrazol-1-yl]propanoic acid (1, 50 mg, 77.83 μmol) in anhydrous DMF (2 mL) were added 5-(6-amino-1-fluoro-3-hydroxy-2-naphthyl)-1,1-dioxo-1,2,5-thiadiazolidin-3-one (2, 37.74 mg, 121.24 μmol), DIPEA (47.01 mg, 363.73 μmol, 63.36 μL) and 1-propanephosphonic anhydride (50% in EtOAc) (0.11 mL, 57.87 mg, 181.86 μmol). The resulting mixture was stirred at RT for 16 h. The solvent was removed under reduced pressure and the residue purified by reverse phase prep HPLC [Purification method: Column: X-Select C18 (150×19 mm) 5 mic, mobile phase: 0.1% TFA in water and MeCN] to afford 3-[4-[[2-(2,6-dioxo-3-piperidyl)-1-oxo-isoindolin-4-yl]oxymethyl]pyrazol-1-yl]-N-[5-fluoro-7-hydroxy-6-(1,1,4-trioxo-1,2,5-thiadiazolidin-2-yl)-2-naphthyl] propanamide (Example 21 14.3 mg, 19.79 μmol, 16% yield) as an off-white solid. LCMS (ES+): m/z 704.0 [M−H]^{- 1}H NMR (400 MHZ, DMSO-d₆) δ 10.96 (s, 1H), 10.49 (s, 1H), 10.28 (s, 1H), 8.15 (s, 1H), 7.89 (s, 1H), 7.85 (d, J=9.0 Hz, 1H), 7.57 (s, 1H), 7.45 (t, J=7.8 Hz, 1H), 7.40 (dd, J=9.1, 1.9 Hz, 1H), 7.35 (d, J=8.1 Hz, 1H), 7.29 (d, J=7.4 Hz, 1H), 6.96 (s, 1H), 5.11-5.04 (m, 3H), 4.46-4.38 (m, 4H), 4.33 (d, J=17.4 Hz, 1H), 4.17 (d, J=17.5 Hz, 1H), 2.96 (t, J=6.8 Hz, 2H), 2.93-2.83 (m, 1H), 2.46-2.37 (m, 2H), 2.01-1.89 (m, 1H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)acetamide (Example 22)

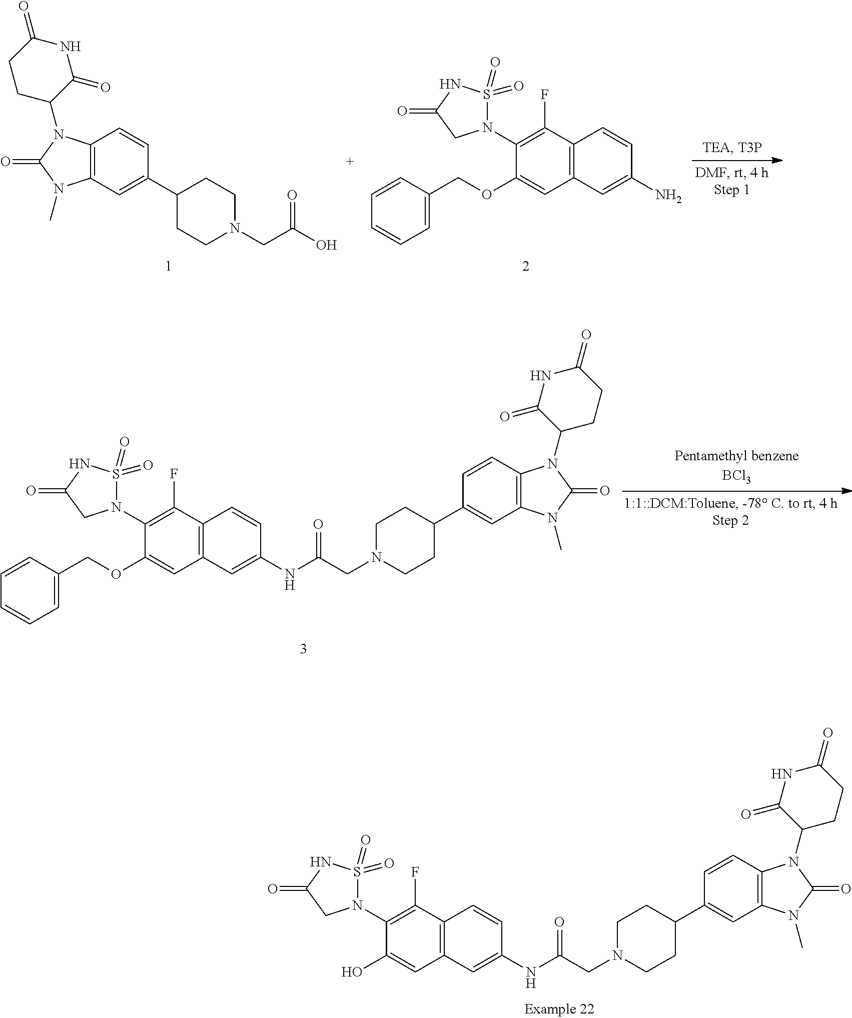 [SMILES: Cn1c(=O)n(C2CCC(=O)NC2=O)c2ccc(C3CCN(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN(CC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)acetamide (3)

Into a 25 mL round bottom flask containing a well-stirred solution of 2-(4-(1-(2,6-dioxopiperidin-3-yl)-3-methyl-2-oxo-2,3-dihydro-1H-benzo[d]imidazol-5-yl)piperidin-1-yl) acetic acid (1, 200 mg, 499.47 μmol) in DMF (2 mL) was added triethylamine (151.62 mg, 1.50 mmol, 208.85 μL) and propylphosphonic anhydride (T₃P) (50% solution in EtOAc) (158.92 mg, 499.47 μmol, 0.3 mL) and the reaction mixture was stirred at RT for 30 min. Then, 5-(6-amino-3-benzyloxy-1-fluoro-2-naphthyl)-1,1-dioxo-1,2,5-thiadiazolidin-3-one (2, 257.44 mg, 499.47 μmol, TFA salt) was added. The reaction mixture was stirred at ambient temperature for 4 h. The volatiles were removed under reduced pressure, and the residue was purified by reverse phase prep HPLC [Purification method: X-Bridge C18 (19×150 mm) 5.0 mic, Mobile Phase A: 0.1% TFA in water; Mobile phase B: MeCN] to obtain N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl) piperidin-1-yl)acetamide (3, 90 mg, 86.22 μmol, 17% yield, TFA salt) as a brown sticky solid. LCMS (ES+): m/z 784.2 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)acetamide (Example 22)

Into a 25 mL round bottom flask containing a well-stirred solution of N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl) acetamide (3, 90 mg, 100.24 μmol, TFA salt) and pentamethylbenzene (44.58 mg, 300.72 μmol, 48.62 μL) in a mixture of DCM (2 mL) and toluene (2 mL) was added boron trichloride (1 M in DCM) (3.00 mmol, 3 mL) dropwise at −78° C. The reaction mixture was stirred at RT for 4 h. The reaction mixture was cooled to −78° C. and quenched by dropwise addition of a 10:1 mixture of DCM and MeOH (5 mL). The volatiles were removed under reduced pressure, and the residue was purified by reverse phase prep HPLC [Purification method: Column: X Bridge C18 (19×150 mm), 5.0 μm; Mobile phase A: 0.1% TFA in water and Mobile phase B: MeCN] to obtain N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)acetamide (Example 22, 17 mg, 20.72 μmol, 21% yield, TFA salt) as an off-white solid. LCMS (ES+): m/z 694.2 [M+H]⁺

¹H NMR (400 MHZ, DMSO-d₆) δ 11.11 (s, 1H), 10.81 (s, 1H), 9.91 (s, 1H), 9.82 (s, 1H), 8.12 (s, 1H), 7.90 (d, J=8.9 Hz, 1H), 7.50-7.44 (m, 1H), 7.11 (d, J=9.6 Hz, 1H), 7.09-7.05 (m, 1H), 7.00 (d, J=4.1 Hz, 1H), 6.94 (d, J=8.0 Hz, 1H), 5.36 (dd, J=12.7, 5.4 Hz, 1H), 4.23 (s, 2H), 4.10 (s, 2H), 3.67 (d, J=11.4 Hz, 2H), 2.98-2.83 (m, 3H), 2.78-2.60 (m, 4H), 2.18-1.93 (m, 6H), 1.51 (d, J=7.0 Hz, 1H), 1.23 (s, 1H).

### 2-[4-[3-(2,6-dioxo-3-piperidyl)-2-oxo-1,3-benzoxazol-6-yl]-1-piperidyl]-N-[5-fluoro-7-hydroxy-6-(1,1,4-trioxo-1,2,5-thiadiazolidin-2-yl)-2-naphthyl]acetamide (Example 23)

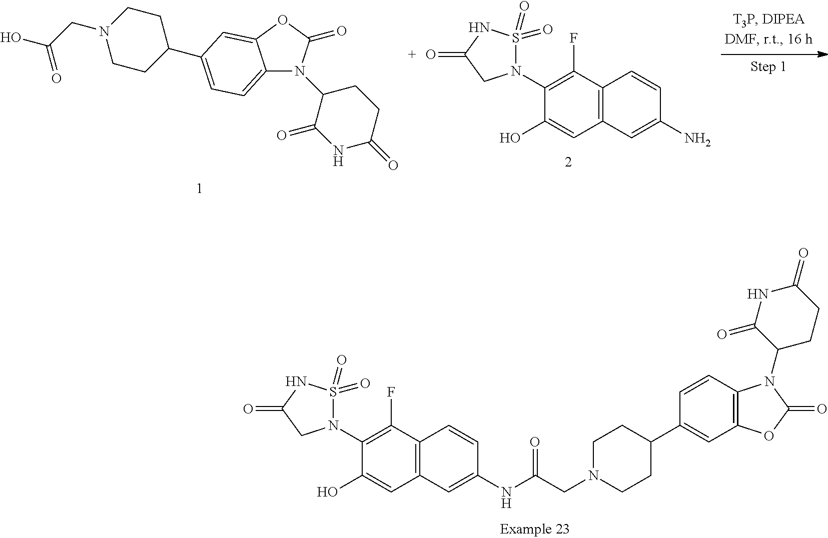 [SMILES: O=C(O)CN1CCC(c2ccc3c(c2)oc(=O)n3C2CCC(=O)NC2=O)CC1 ; Nc1ccc2c(F)c(N3CC(=O)NS3(=O)=O)c(O)cc2c1 ; O=C1CCC(n2c(=O)oc3cc(C4CCN(CC(=O)Nc5ccc6c(F)c(N7CC(=O)NS7(=O)=O)c(O)cc6c5)CC4)ccc32)C(=O)N1]

### Step 1: 2-[4-[3-(2,6-dioxo-3-piperidyl)-2-oxo-1,3-benzoxazol-6-yl]-1-piperidyl]-N-[5-fluoro-7-hydroxy-6-(1,1,4-trioxo-1,2,5-thiadiazolidin-2-yl)-2-naphthyl]acetamide (Example 23)

Into a 10 mL single neck round bottom flask containing a well-stirred solution 2-[4-[3-(2,6-dioxo-3-piperidyl)-2-oxo-1,3-benzoxazol-6-yl]-1-piperidyl]acetic acid (1, 35 mg, 0.069 mmol, TFA salt) and 5-(6-amino-1-fluoro-3-hydroxy-2-naphthyl)-1,1-dioxo-1,2,5-thiadiazolidin-3-one (2, 24.27 mg, 0.069 mmol, HCl salt) in DMF (0.5 mL) were added DIPEA (9.02 mg, 0.069 mmol, 12.16 μL) and propylphosphonic anhydride (T₃P) solution (≥50 wt. % in EtOAc) (22.21 mg, 0.069 mmol, 50 μL) at 0° C. The resulting mixture was stirred at RT for 16 h. The reaction mixture was diluted with water (2 mL) and concentrated under reduced pressure, and the residue was purified by reverse phase prep HPLC [Purification method: Column: X-BRIDGE C18 (19×150 mm) 5.0 μm with Mobile phase A: 0.1% TFA in water; Mobile phase B: Acetonitrile] to afford 2-[4-[3-(2,6-dioxo-3-piperidyl)-2-oxo-1,3-benzoxazol-6-yl]-1-piperidyl]-N-[5-fluoro-7-hydroxy-6-(1,1,4-trioxo-1,2,5-thiadiazolidin-2-yl)-2-naphthyl] acetamide (Example 23, 12 mg, 0.014 mmol, 21% yield, TFA salt) as an off white solid. LCMS (ES+): m/z 681.0 [M+H]^{+ 1}H NMR (400 MHz, DMSO-d₆) δ 11.23 (s, 1H), 10.80 (s, 1H), 9.97 (s, 1H), 9.84 (s, 1H), 8.12 (s, 1H), 7.91 (d, J=8.9 Hz, 1H), 7.47 (d, J=9.1 Hz, 1H), 7.32 (s, 1H), 7.24 (d, J=8.1 Hz, 1H), 7.12 (d, J=8.4 Hz, 1H), 7.00 (s, 1H), 5.37 (dd, J=13.0, 5.3 Hz, 1H), 4.23 (s, 2H), 4.13 (s, 2H), 3.72-3.61 (m, 2H), 3.30-3.22 (m, 4H), 2.98-2.85 (m, 2H), 2.76-2.62 (m, 2H), 2.23-2.12 (m, 1H), 2.09-1.94 (m, 4H), 1.02-0.80 (m, 1H).

### 2-[4-[1-(2,6-dioxo-3-piperidyl)-3-isopropyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Example 24)

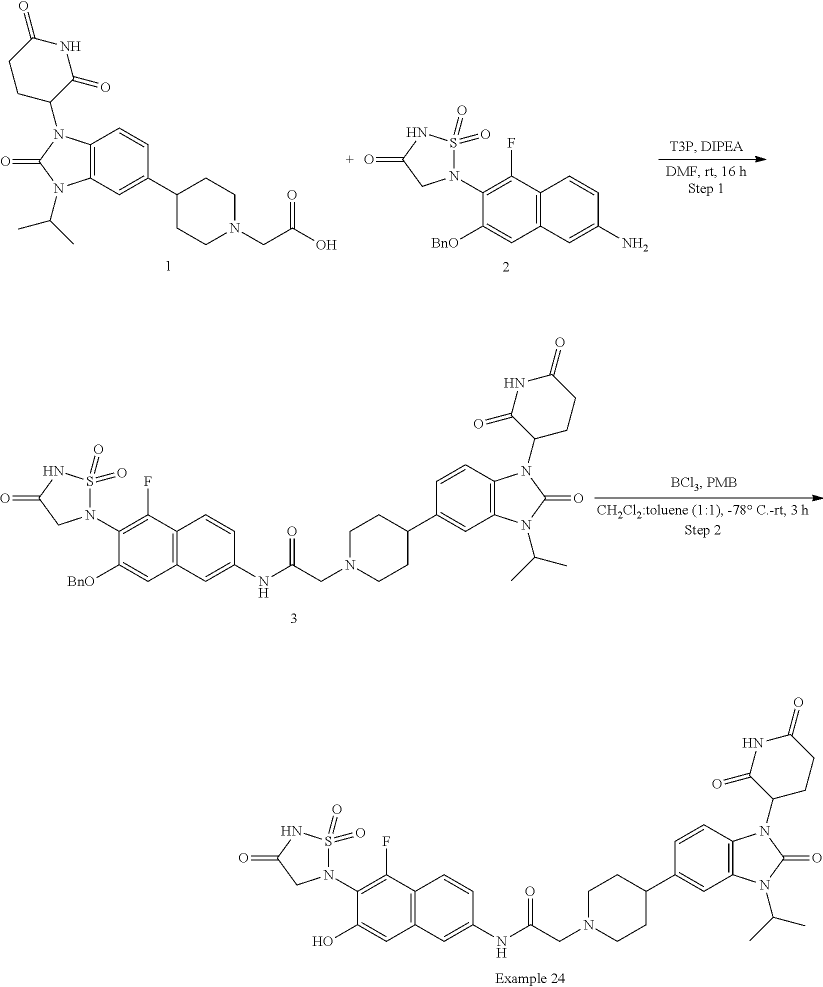 [SMILES: CC(C)n1c(=O)n(C2CCC(=O)NC2=O)c2ccc(C3CCN(CC(=O)O)CC3)cc21 ; Nc1ccc2c(F)c(N3CC(=O)NS3(=O)=O)c(OCc3ccccc3)cc2c1 ; CC(C)n1c(=O)n(C2CCC(=O)NC2=O)c2ccc(C3CCN(CC(=O)Nc4ccc5c(F)c(N6CC(=O)NS6(=O)=O)c(OCc6ccccc6)cc5c4)CC3)cc21 ; CC(C)n1c(=O)n(C2CCC(=O)NC2=O)c2ccc(C3CCN(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-isopropyl-2-oxo-benzimidazol-5-yl]-1-piperidyl] acetamide (3)

Into a 25 mL single neck round bottom flask containing a well-stirred solution of 2-[4-[1-(2,6-dioxo-3-piperidyl)-3-isopropyl-2-oxo-benzimidazol-5-yl]-1-piperidyl]acetic acid (1, 220 mg, 513.44 μmol, TFA salt) in anhydrous DMF (5 mL) were added 5-(6-amino-3-benzyloxy-1-fluoro-2-naphthyl)-1,1-dioxo-1,2,5-thiadiazolidin-3-one (2, 206.10 mg, 513.44 μmol, TFA salt), DIPEA (199.08 mg, 1.54 mmol, 268.30 μL) and 1-propanephosphonic anhydride (50% in EtOAc) (0.49 mL, 245.05 mg, 770.16 μmol). After 16 h, the volatiles were removed under reduced pressure and the residue purified by reverse-phase preparative HPLC [Column: C18 aq gold (19×150) mm, 5 micron; Mobile phase A: 0.1% TFA in water and Mobile phase B: MeCN] to obtain N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-isopropyl-2-oxo-benzimidazol-5-yl]-1-piperidyl]acetamide (3, 200 mg, 192.25 μmol, 37% yield, TFA salt) as an off-white solid. LCMS (ES+): m/z 812.3 [M+H]⁺

### Step 2: 2-[4-[1-(2,6-dioxo-3-piperidyl)-3-isopropyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl] acetamide (Example 24)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-isopropyl-2-oxo-benzimidazol-5-yl]-1-piperidyl]acetamide (3, 200 mg, 246.34 μmol, TFA salt) in anhydrous DCM (5 mL) and anhydrous toluene (5 mL) was added pentamethylbenzene (36.52 mg, 246.34 μmol). The reaction mixture was cooled to −78° C. and BCl₃ (1 M in methylene chloride) (2.46 mL, 2.46 mmol) was added dropwise. The resulting solution was stirred at ambient temperature for 3 h. The reaction mixture was quenched with 5% MeOH in DCM (5 mL) at −78° C. and the volatiles removed under reduced pressure. The residue was purified by reverse-phase preparative HPLC [Column: C18 aq gold (19×150) mm, 5 micron; Mobile phase A: 0.1% TFA in water and Mobile phase B: MeCN] to obtain 2-[4-[1-(2,6-dioxo-3-piperidyl)-3-isopropyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]acetamide (Example 24, 45 mg, 51.95 μmol, 21% yield, TFA salt) as an off-white solid. LCMS (ES+): m/z 722.7 [M+H]^{+ 1}H NMR (400 MHZ, DMSO-d₆) δ 11.09 (s, 1H), 10.82 (s, 1H), 10.18 (s, 1H), 9.77 (s, 1H), 8.12 (d, J=1.9 Hz, 1H), 7.92 (d, J=9.0 Hz, 1H), 7.48 (dd, J=9.1, 2.0 Hz, 1H), 7.22-7.18 (m, 1H), 7.07 (d, J=8.1 Hz, 1H), 7.01 (s, 1H), 6.93 (d, J=8.1 Hz, 1H), 5.33 (dd, J=12.8, 5.4 Hz, 1H), 4.63 (p, J=6.9 Hz, 1H), 3.68 (d, J=11.5 Hz, 2H), 3.32-3.23 (m, 2H), 2.97-2.83 (m, 2H), 2.75-2.58 (m, 2H), 2.19-1.93 (m, 5H), 1.49 (s, 3H), 1.47 (s, 3H).

### 2-[4-[1-(2,6-dioxo-3-piperidyl)-3-methyl-2-oxo-benzimidazol-4-yl]-1-piperidyl]-N-[5-fluoro-7-hydroxy-6-(1,1,4-trioxo-1,2,5-thiadiazolidin-2-yl)-2-naphthyl]acetamide (Example 25)

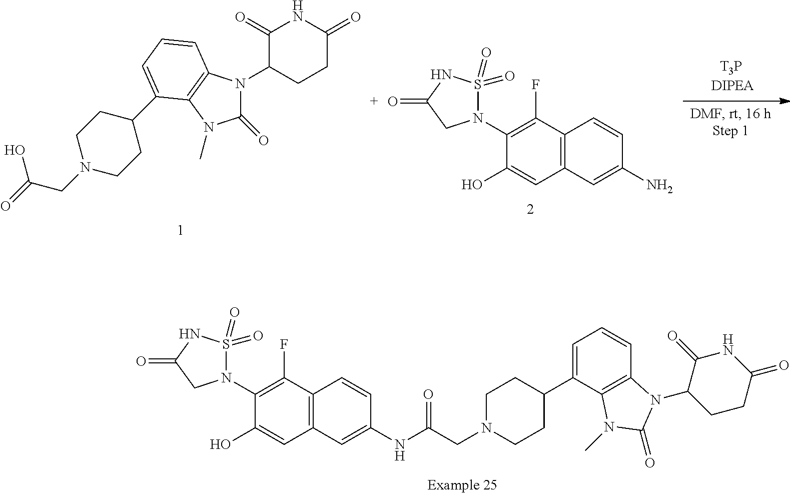 [SMILES: Cn1c(=O)n(C2CCC(=O)NC2=O)c2cccc(C3CCN(CC(=O)O)CC3)c21 ; Nc1ccc2c(F)c(N3CC(=O)NS3(=O)=O)c(O)cc2c1 ; Cn1c(=O)n(C2CCC(=O)NC2=O)c2cccc(C3CCN(CC(=O)Nc4ccc5c(F)c(N6CC(=O)NS6(=O)=O)c(O)cc5c4)CC3)c21]

### Step 1: 2-[4-[1-(2,6-dioxo-3-piperidyl)-3-methyl-2-oxo-benzimidazol-4-yl]-1-piperidyl]-N-[5-fluoro-7-hydroxy-6-(1,1,4-trioxo-1,2,5-thiadiazolidin-2-yl)-2-naphthyl]acetamide (Example 25)

2-[4-[1-(2,6-dioxo-3-piperidyl)-3-methyl-2-oxo-benzimidazol-4-yl]-1-piperidyl]acetic acid (1, 50 mg, 124.87 μmol) and 5-(6-amino-1-fluoro-3-hydroxy-2-naphthyl)-1,1-dioxo-1,2,5-thiadiazolidin-3-one (2, 38.87 mg, 124.87 μmol) in DMF (500 μL) were treated with DIPEA (64.55 mg, 499.47 μmol, 87.00 μL) and cooled to 0° C.) Propylphosphonic anhydride (50 wt % in EtOAc) (239.89 mg, 374.60 μmol, 480 μL) was added and the reaction allowed to come to rt. After 16 h, the mixture was purified directly by reverse phase chromatography, eluting with 5-100% acetonitrile in water with 0.1% TFA modifier to afford 2-[4-[1-(2,6-dioxo-3-piperidyl)-3-methyl-2-oxo-benzimidazol-4-yl]-1-piperidyl]-N-[5-fluoro-7-hydroxy-6-(1,1,4-trioxo-1,2,5-thiadiazolidin-2-yl)-2-naphthyl]acetamide (Example 25, 17.7 mg, 19.94 μmol, 16% yield, TFA salt) as a grey solid. LCMS: 694.5 [M+H]^{+ 1}H NMR (400 MHZ, DMSO-d₆) δ 11.10 (s, 1H), 10.79 (s, 1H), 9.84 (s, 2H), 8.12 (s, 1H), 7.91 (d, J=9.0 Hz, 1H), 7.51-7.45 (m, 1H), 7.13-6.96 (m, 4H), 5.39 (dd, J=12.6, 5.5 Hz, 1H), 4.23 (s, 1H), 4.10 (s, 2H), 3.71-3.56 (m, 6H), 2.95-2.84 (m, 1H), 2.79-2.58 (m, 2H), 2.25-2.12 (m, 2H), 2.10-1.95 (m, 3H), 1.00-0.90 (m, 2H), 0.89-0.81 (m, 1H).

### 2-[4-[1-(2,6-dioxo-3-piperidyl)-2-oxo-benzo[cd]indol-6-yl]-1-piperidyl]-N-[5-fluoro-7-hydroxy-6-(1,1,4-trioxo-1,2,5-thiadiazolidin-2-yl)-2-naphthyl]acetamide (Example 26)

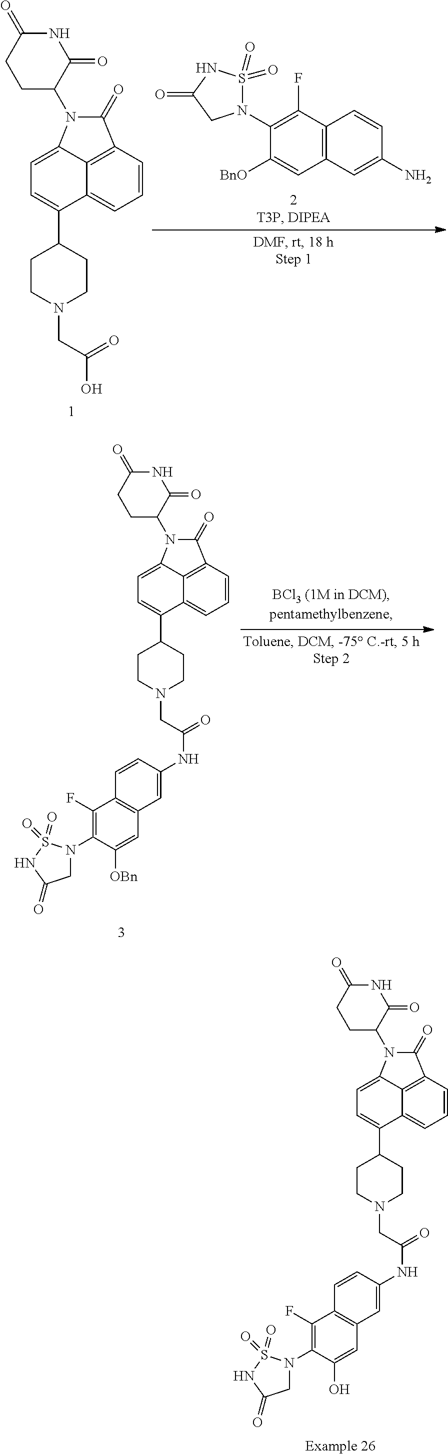 [SMILES: O=C(O)CN1CCC(c2ccc3c4c(cccc24)C(=O)N3C2CCC(=O)NC2=O)CC1 ; Nc1ccc2c(F)c(N3CC(=O)NS3(=O)=O)c(OCc3ccccc3)cc2c1 ; O=C1CCC(N2C(=O)c3cccc4c(C5CCN(CC(=O)Nc6ccc7c(F)c(N8CC(=O)NS8(=O)=O)c(OCc8ccccc8)cc7c6)CC5)ccc2c34)C(=O)N1 ; O=C1CCC(N2C(=O)c3cccc4c(C5CCN(CC(=O)Nc6ccc7c(F)c(N8CC(=O)NS8(=O)=O)c(O)cc7c6)CC5)ccc2c34)C(=O)N1]

### Step 1: Preparation of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-2-oxo-benzo[cd]indol-6-yl]-1-piperidyl] acetamide (3)

Into a 10 mL single neck round bottom flask containing a well-stirred solution of 5-(6-amino-3-benzyloxy-1-fluoro-2-naphthyl)-1,1-dioxo-1,2,5-thiadiazolidin-3-one (2, 80 mg, 199.30 μmol, TFA salt) and 2-[4-[1-(2,6-dioxo-3-piperidyl)-2-oxo-benzo[cd]indol-6-yl]-1-piperidyl]acetic acid (1, 92.39 mg, 219.23 μmol, TFA salt) in DMF (2 mL) was added N,N-diisopropylethylamine (148.40 mg, 1.15 mmol, 0.2 mL) and a propylphosphonic anhydride (T₃P) (50 wt % in EtOAc) (23.20 mg, 219.23 μmol, 0.05 mL) at RT. The reaction mixture was stirred at RT for 18 h. The volatiles were removed under reduced pressure, and the residue was purified by reverse phase prep HPLC [Purification method: Column: X-BRIDGE C8 (19×150 mm), 5 micron; Mobile phase A: 0.1% TFA in H₂O; Mobile phase B: MeCN] to obtain N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-2-oxo-benzo[cd]indol-6-yl]-1-piperidyl]acetamide (3, 50 mg, 49.70 μmol, 25% yield, TFA salt) as a pale yellow solid. LCMS (ES+): m/z 805.0 [M+H]⁺

### Step 2: Preparation of 2-[4-[1-(2,6-dioxo-3-piperidyl)-2-oxo-benzo[cd]indol-6-yl]-1-piperidyl]-N-[5-fluoro-7-hydroxy-6-(1,1,4-trioxo-1,2,5-thiadiazolidin-2-yl)-2-naphthyl] acetamide (Example 26)

Into a 10 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-2-oxo-benzo[cd]indol-6-yl]-1-piperidyl]acetamide (3, 50 mg, 62.12 μmol, TFA salt) in toulene (1 mL) and DCM (1 mL) was added pentamethylbenzene (46.05 mg, 310.62 μmol, 50.22 μL) at RT. Boron trichloride (1 M in DCM) (0.5 mmol, 0.5 mL) was then added at −78° C. The reaction mixture was stirred for 5 h at RT. The reaction mixture was cooled to −78° C. and quenched with 10% MeOH in DCM (1.5 mL). The volatiles were removed under reduced pressure, and the residue was purified by reverse phase prep HPLC [Purification method: Column: X-BRIDGE C8 (19×150 mm), 5 mic; Mobile phase A: 0.1% TFA in H₂O; Mobile phase B: MeCN] to obtain 2-[4-[1-(2,6-dioxo-3-piperidyl)-2-oxo-benzo[cd]indol-6-yl]-1-piperidyl]-N-[5-fluoro-7-hydroxy-6-(1,1,4-trioxo-1,2,5-thiadiazolidin-2-yl)-2-naphthyl] acetamide (Example 26, 10 mg, 11.62 μmol, 19% yield, TFA salt) as a pale yellow solid. LCMS (ES+): m/z 712.8 [M−H]^{- 1}H NMR (400 MHz, DMSO-d₆) δ 11.14 (s, 1H), 10.82 (s, 1H), 10.07 (s, 1H), 9.97 (s, 1H), 8.50 (d, J=8.4 Hz, 1H), 8.16-8.10 (m, 2H), 7.94-7.87 (m, 2H), 7.47 (dd, J=9.1, 2.0 Hz, 1H), 7.38 (d, J=7.6 Hz, 1H), 7.18-7.11 (m, 1H), 7.03-6.98 (m, 1H), 5.44 (dd, J=12.7, 5.4 Hz, 1H), 4.26 (s, 2H), 4.13 (s, 2H), 3.77-3.63 (m, 5H), 3.03-2.89 (m, 1H), 2.82-2.63 (m, 2H), 2.31-2.18 (m, 2H), 2.15-2.05 (m, 3H).

### 2-[3-[[1-(2,6-dioxo-3-piperidyl)-3-methyl-2-oxo-benzimidazol-5-yl]amino]azetidin-1-yl]-N-[5-fluoro-7-hydroxy-6-(1,1,4-trioxo-1,2,5-thiadiazolidin-2-yl)-2-naphthyl]acetamide (Example 27)

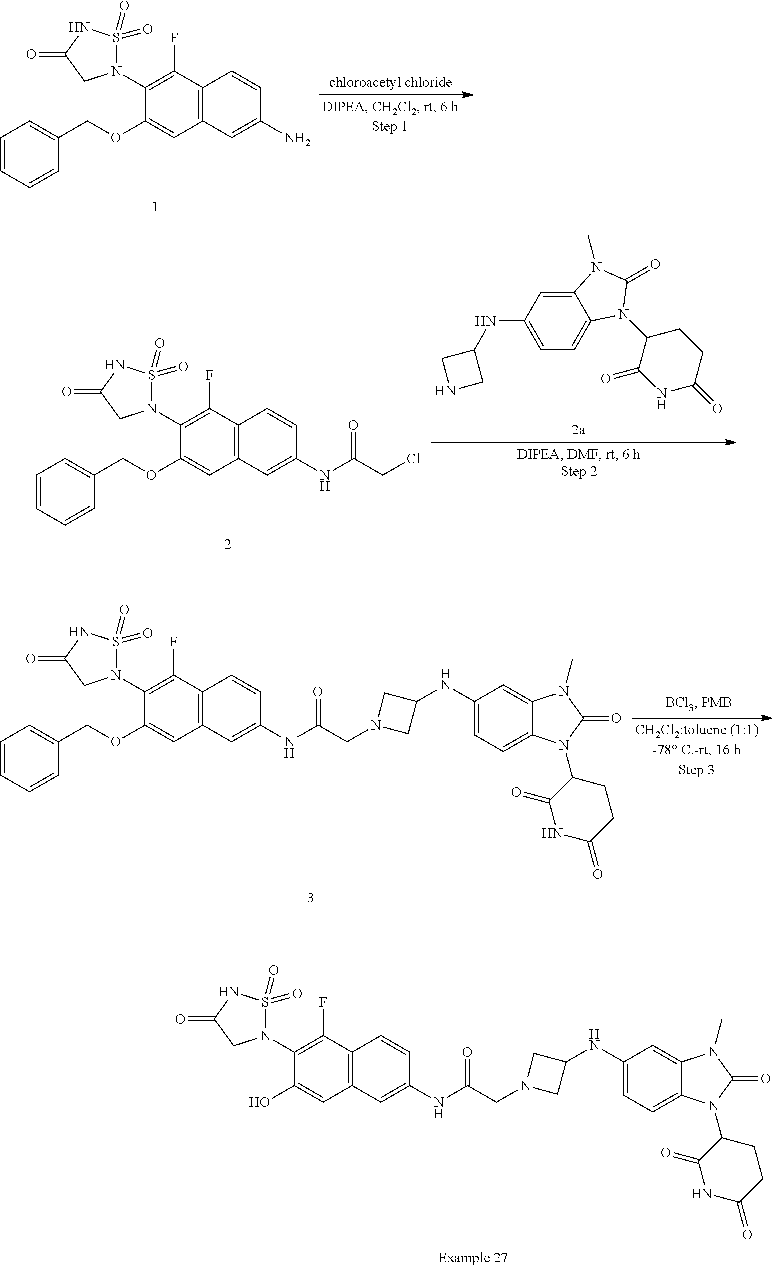 [SMILES: Nc1ccc2c(F)c(N3CC(=O)NS3(=O)=O)c(OCc3ccccc3)cc2c1 ; O=C(CCl)Nc1ccc2c(F)c(N3CC(=O)NS3(=O)=O)c(OCc3ccccc3)cc2c1 ; Cn1c(=O)n(C2CCC(=O)NC2=O)c2ccc(NC3CNC3)cc21 ; Cn1c(=O)n(C2CCC(=O)NC2=O)c2ccc(NC3CN(CC(=O)Nc4ccc5c(F)c(N6CC(=O)NS6(=O)=O)c(OCc6ccccc6)cc5c4)C3)cc21 ; Cn1c(=O)n(C2CCC(=O)NC2=O)c2ccc(NC3CN(CC(=O)Nc4ccc5c(F)c(N6CC(=O)NS6(=O)=O)c(O)cc5c4)C3)cc21]

### Step 1: N-[7-benzyloxy-5-fluoro-6-(1,1,4-trioxo-1,2,5-thiadiazolidin-2-yl)-2-naphthyl]-2-chloro-acetamide (2)

To a 50 mL single neck round bottom flask containing a well-stirred solution of 5-(6-amino-3-benzyloxy-1-fluoro-2-naphthyl)-1,1-dioxo-1,2,5-thiadiazolidin-3-one (1, 300 mg, 582.03 μmol, TFA salt) in DCM (10 mL) were added DIPEA (225.67 mg, 1.75 mmol, 304.13 μL) and chloroacetyl chloride (100.77 mg, 892.22 μmol, 70.96 μL) at 0° C. The resulting solution was stirred at RT for 16 h. The reaction mixture was concentrated under reduced pressure, and the residue was azeotroped with toluene (2×20 mL) and purified by reverse phase chromatography [biotage C18 (60 g) column. Mobile phase A: 0.1% TFA in water; Mobile phase B: Acetonitrile] to afford N-[7-benzyloxy-5-fluoro-6-(1,1,4-trioxo-1,2,5-thiadiazolidin-2-yl)-2-naphthyl]-2-chloro-acetamide (2, 140 mg, 175.40 μmol, 30% yield. TFA Salt) as an off-white solid. LCMS (ES−): m/z 476.0 [M−H]⁻

### Step 2: N-[7-benzyloxy-5-fluoro-6-(1,1,4-trioxo-1,2,5-thiadiazolidin-2-yl)-2-naphthyl]-2-[3-[[1-(2,6-dioxo-3-piperidyl)-3-methyl-2-oxo-benzimidazol-5-yl]amino]azetidin-1-yl] acetamide (3)

Into a 50 mL single neck round bottom flask containing a well-stirred solution of 3-[5-(azetidin-3-ylamino)-3-methyl-2-oxo-benzimidazol-1-yl]piperidine-2,6-dione (2a, 129.89 mg, 292.95 μmol, TFA salt) and N-[7-benzyloxy-5-fluoro-6-(1,1,4-trioxo-1,2,5-thiadiazolidin-2-yl)-2-naphthyl]-2-chloro-acetamide (2, 140 mg, 292.95 μmol) in anhydrous DMF (5 mL) was added DIPEA (189.31 mg, 1.46 mmol, 255.13 μL). The resulting mixture was stirred at RT for 16 h. The reaction mixture was concentrated under reduced pressure, and the residue was purified by reverse-phase chromatography [Column: Biotage C18 (60 g) with Solvent A: 0.1% TFA in water; Solvent B: Acetonitrile] to afford N-[7-benzyloxy-5-fluoro-6-(1,1,4-trioxo-1,2,5-thiadiazolidin-2-yl)-2-naphthyl]-2-[3-[[1-(2,6-dioxo-3-piperidyl)-3-methyl-2-oxo-benzimidazol-5-yl]amino]azetidin-1-yl]acetamide (3, 100 mg, 83.81 μmol, 29% yield, TFA salt) as an off-white solid. LCMS (ES+): m/z 771.2 [M+H]⁺

### Step 3: 2-[3-[[1-(2,6-dioxo-3-piperidyl)-3-methyl-2-oxo-benzimidazol-5-yl]amino]azetidin-1-yl]-N-[5-fluoro-7-hydroxy-6-(1,1,4-trioxo-1,2,5-thiadiazolidin-2-yl)-2-naphthyl]acetamide (Example 27)

Into a 10 mL single neck round bottom flask containing a well-stirred solution of N-[7-benzyloxy-5-fluoro-6-(1,1,4-trioxo-1,2,5-thiadiazolidin-2-yl)-2-naphthyl]-2-[3-[[1-(2,6-dioxo-3-piperidyl)-3-methyl-2-oxo-benzimidazol-5-yl]amino]azetidin-1-yl]acetamide (3, 100 mg, 113.02 μmol, TFA salt) in a mixture of anhydrous toluene (2 mL) and anhydrous DCM (2 mL) at ambient temperature was added pentamethylbenzene (83.78 mg, 565.09 μmol, 91.36 μL). Then the reaction mixture was cooled to −78° C., and BCl₃ (1.0 M in methylene chloride) (2.5 mmol, 2.5 mL) was added dropwise to the reaction. The resulting mixture was stirred at RT for 16 h. The reaction mixture was cooled to −78° C. and quenched with 5% MeOH/DCM (5 mL). Excess solvents were removed under reduced pressure, and the residue was purified by reverse phase prep HPLC [Column: AGILENT 5PREP-C₁₈ (50×21.2) mm; Mobile phase A: 0.1% Ammonium acetate in MQ-water; Mobile phase B: Acetonitrile] to afford 2-[3-[[1-(2,6-dioxo-3-piperidyl)-3-methyl-2-oxo-benzimidazol-5-yl]amino]azetidin-1-yl]-N-[5-fluoro-7-hydroxy-6-(1,1,4-trioxo-1,2,5-thiadiazolidin-2-yl)-2-naphthyl]acetamide (Example 27, 10 mg, 14.39 μmol, 13% yield) as a beige solid. LCMS (ES−): m/z 679.1 [M−H]^{- 1}H NMR (400 MHZ, DMSO-d₆) δ 11.05 (s, 1H), 10.69 (s, 1H), 9.84 (s, 1H), 8.08 (d, J=2.1 Hz, 1H), 7.88 (d, J=9.0 Hz, 1H), 7.43 (dd, J=9.1, 2.0 Hz, 1H), 7.03-6.96 (m, 1H), 6.88 (d, J=8.4 Hz, 1H), 6.39 (s, 1H), 6.30-6.24 (m, 1H), 6.11 (s, 1H), 5.26 (dd, J=12.7, 5.4 Hz, 1H), 4.58 (s, 2H), 4.46-4.27 (m, 2H), 4.08 (s, 2H), 4.01 (s, 2H), 3.29 (s, 3H), 2.95-2.81 (m, 1H), 2.73-2.56 (m, 2H), 2.02-1.91 (m, 1H).

### 2-[4-[3-(2,6-dioxo-3-piperidyl)-1-methyl-indazol-6-yl]-1-piperidyl]-N-[5-fluoro-7-hydroxy-6-(1,1,4-trioxo-1,2,5-thiadiazolidin-2-yl)-2-naphthyl]acetamide (Example 28)

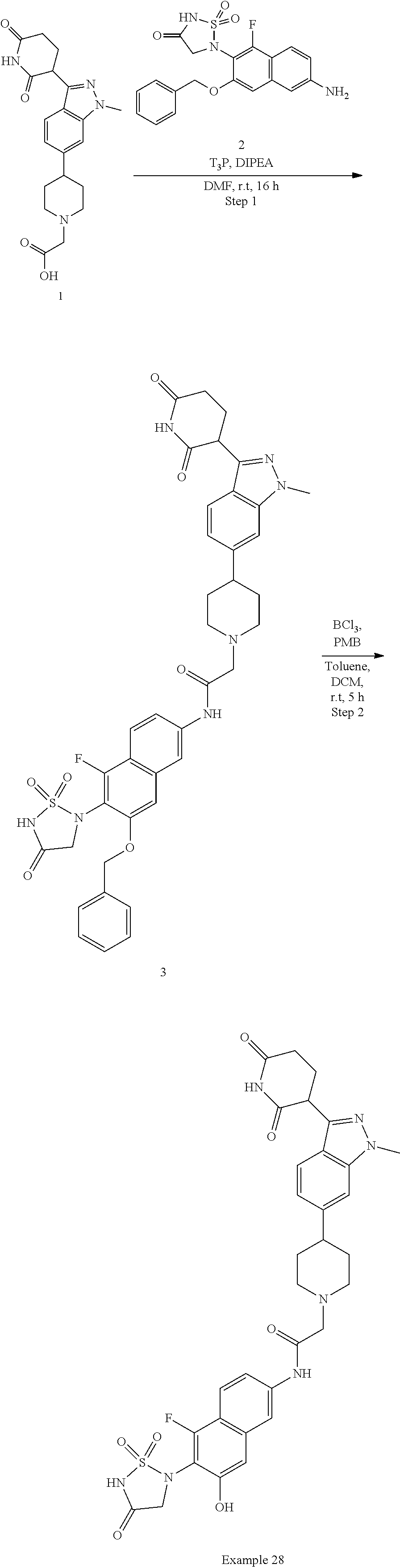

### Step 1: N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-indazol-6-yl]-1-piperidyl]acetamide (3)

Into a 25 mL single neck round bottom flask containing a well-stirred solution of 5-(6-amino-3-benzyloxy-1-fluoro-2-naphthyl)-1,1-dioxo-1,2,5-thiadiazolidin-3-one (2, 260 mg, 647.72 μmol) and 2-[4-[3-(2,6-dioxo-3-piperidyl)-1-methyl-indazol-6-yl]-1-piperidyl]acetic acid (1, 249.00 mg, 647.72 μmol) in anhydrous DMF (4 mL) were added propanephosphonic anhydride (T3P) (50% in EtOAc) (618.27 mg, 1.94 mmol, 1.37 mL) and DIPEA (1.11 g, 8.61 mmol, 1.5 mL). The resulting mixture was stirred at RT for 16 h. Excess solvent was removed under reduced pressure and the residue was purified by reverse phase prep HPLC [Purification method: Column: SYNERGI (250×30 mm), 4 micron; Mobile phase A: 0.1% TFA in water; Mobile phase B: Acetonitrile] to afford N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-indazol-6-yl]-1-piperidyl] acetamide (3, 90 mg, 98.66 μmol, 15% yield, TFA salt) as an off-white solid. LCMS (ES+): m/z 768.3 [M+H]⁺

### Step 2: 2-[4-[3-(2,6-dioxo-3-piperidyl)-1-methyl-indazol-6-yl]-1-piperidyl]-N-[5-fluoro-7-hydroxy-6-(1,1,4-trioxo-1,2,5-thiadiazolidin-2-yl)-2-naphthyl]acetamide (Example 28)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[3-(2,6-dioxo-3-piperidyl)-1-methyl-indazol-6-yl]-1-piperidyl]acetamide (3, 80 mg, 104.19 μmol) in anhydrous DCM (3.0 mL) and anhydrous toluene (3.0 mL) was added pentamethylbenzene (77.23 mg, 520.95 μmol, 84.22 μL). The mixture was cooled to −78° C. and boron trichloride (1 M in DCM) (4.5 mmol, 4.5 mL) was added. The reaction mixture was stirred at RT for 5 h. The mixture was diluted with DCM (10 mL) and the solvent was removed under reduced pressure and azeotroped with toluene (2×4 mL). The residue was purified by reverse phase prep HPLC [Column: X-BRIDGE C8 (19×150 mm), 5 micron; Mobile phase A: 0.1% Formic acid in water; Mobile phase B: Acetonitrile] to afford 2-[4-[3-(2,6-dioxo-3-piperidyl)-1-methyl-indazol-6-yl]-1-piperidyl]-N-[5-fluoro-7-hydroxy-6-(1,1,4-trioxo-1,2,5-thiadiazolidin-2-yl)-2-naphthyl]acetamide (Example 28, 24 mg, 31.51 μmol, 30% yield, formate salt) as an off-white solid. LCMS (ES−): m/z 676.2 [M−H]^{- 1}H NMR (400 MHz, DMSO-d₆) δ 10.88 (s, 1H), 10.76 (s, 1H), 10.05 (s, 1H), 8.10 (d, J=2.2 Hz, 1H), 7.89 (d, J=8.9 Hz, 1H), 7.67 (d, J=8.4 Hz, 1H), 7.49-7.41 (m, 2H), 7.08 (d, J=8.5 Hz, 1H), 6.99 (s, 1H), 4.34 (dd, J=9.9, 5.1 Hz, 1H), 4.16 (d, J=12.6 Hz, 2H), 4.11 (s, 2H), 3.98 (s, 3H), 3.31-3.18 (m, 2H), 3.06-2.94 (m, 1H), 2.74-2.55 (m, 2H), 2.42-2.27 (m, 1H), 2.22-2.00 (m, 5H).

### N-[2-[4-[1-(2,6-dioxo-3-piperidyl)-3-methyl-2-oxo-benzimidazol-5-yl]-1-piperidyl]ethyl]-5-fluoro-7-hydroxy-6-(1,1,4-trioxo-1,2,5-thiadiazolidin-2-yl)naphthalene-2-carboxamide (Example 29)

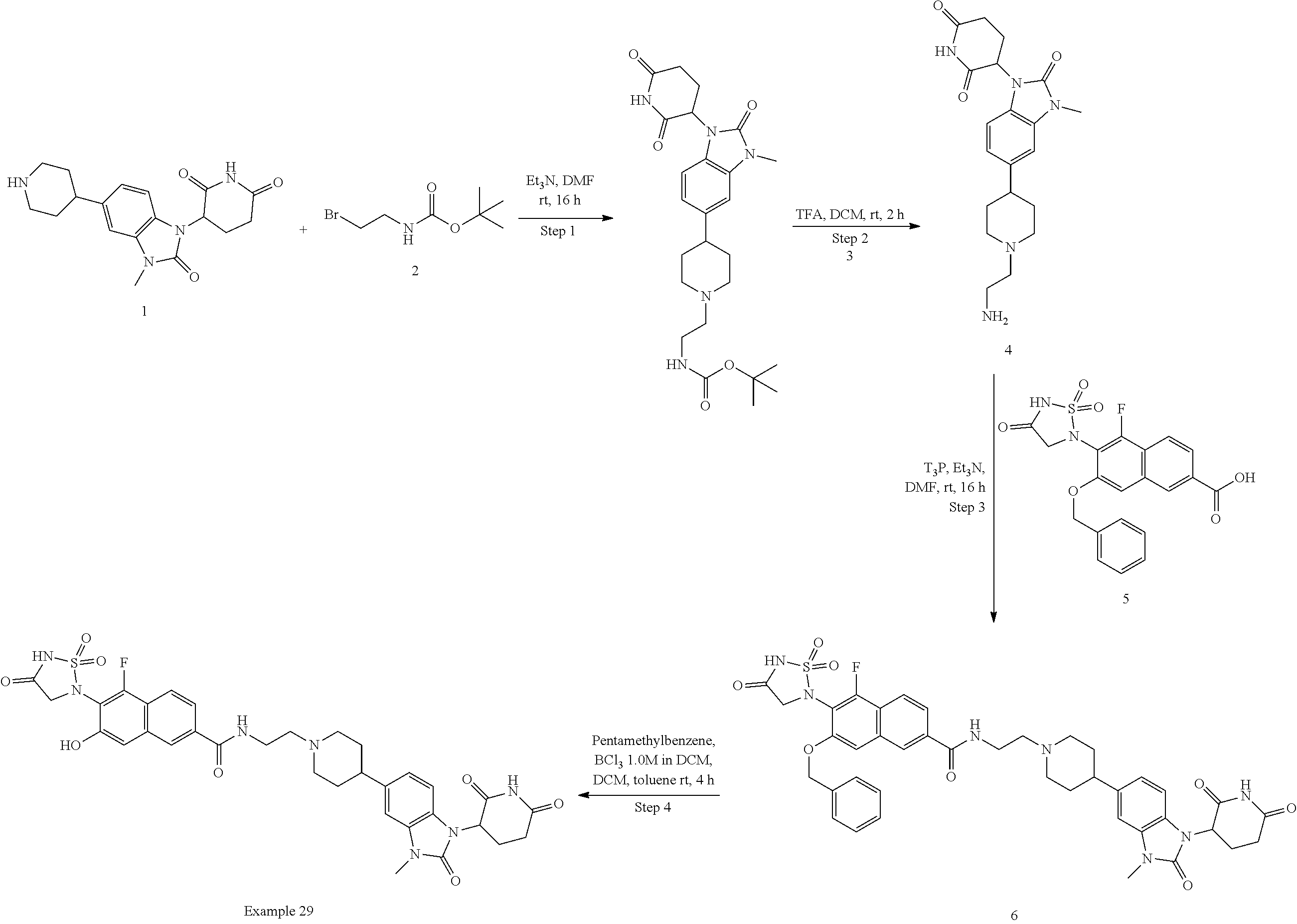

### Step 1: tert-butyl N-[2-[4-[1-(2,6-dioxo-3-piperidyl)-3-methyl-2-oxo-benzimidazol-5-yl]-1-piperidyl] ethyl]carbamate (3)

Into a 20 mL capped vial containing a well-stirred solution of 3-[3-methyl-2-oxo-5-(4-piperidyl)benzimidazol-1-yl]piperidine-2,6-dione (1, 100 mg, 292.06 μmol) in DMF (2 mL) was added triethylamine (88.66 mg, 876.19 μmol, 122.12 μL) followed by tert-butyl N-(2-bromoethyl)carbamate (2, 65.45 mg, 292.06 μmol), and the reaction mixture was stirred at RT for 16 h. The reaction was quenched with ice water and extracted with ethyl acetate (2×100 mL). The combined organic layer was washed with water (100 mL) followed by brine (50 mL) and dried over Na₂SO₄, filtered, and concentrated under reduced pressure to obtain tert-butyl N-[2-[4-[1-(2,6-dioxo-3-piperidyl)-3-methyl-2-oxo-benzimidazol-5-yl]-1-piperidyl]ethyl]carbamate (3, 83 mg, 143.58 μmol, 49% yield) as a brown solid. This material was used in the next step without further purification. LCMS (ES+): m/z 486.3 [M+H]⁺

### Step 2: 3-[5-[1-(2-aminoethyl)-4-piperidyl]-3-methyl-2-oxo-benzimidazol-1-yl]piperidine-2,6-dione (4)

Into a 25 mL single neck round bottom flask containing a well-stirred suspension of tert-butyl N-[2-[4-[1-(2,6-dioxo-3-piperidyl)-3-methyl-2-oxo-benzimidazol-5-yl]-1-piperidyl]ethyl]carbamate (3, 80 mg, 164.75 μmol) in DCM (2 mL), was added TFA (93.93 mg, 823.77 μmol, 63.47 μL) at 0° C. dropwise. The resulting mixture was stirred at RT for 2 h. The volatiles were removed under reduced pressure to get a light brown residue which was triturated with diethyl ether (10 mL) to afford 3-[5-[1-(2-aminoethyl)-4-piperidyl]-3-methyl-2-oxo-benzimidazol-1-yl]piperidine-2,6-dione (4, 60 mg, 107.38 μmol, 65% yield, TFA salt) as a brown semisolid. This material was used in the next step without further purification. LCMS (ES+): m/z 386.2 [M+H]⁺

### Step 3: 7-benzyloxy-N-[2-[4-[1-(2,6-dioxo-3-piperidyl)-3-methyl-2-oxo-benzimidazol-5-yl]-1-piperidyl] ethyl]-5-fluoro-6-(1,1,4-trioxo-1,2,5-thiadiazolidin-2-yl)naphthalene-2-carboxamide (6)

Into a 10 mL single neck round bottom flask containing a well-stirred solution of 7-benzyloxy-5-fluoro-6-(1,1,4-trioxo-1,2,5-thiadiazolidin-2-yl)naphthalene-2-carboxylic acid (5, 40 mg, 92.94 μmol) in DMF (2.5 mL), was added triethylamine (37.62 mg, 371.74 μmol, 51.81 μL) and propylphosphonic anhydride (T3P) (50% in EtOAc) (59.14 mg, 185.87 μmol, 130 μL). The reaction mixture was stirred at RT for 15 minutes before 3-[5-[1-(2-aminoethyl)-4-piperidyl]-3-methyl-2-oxo-benzimidazol-1-yl]piperidine-2,6-dione (4, 53.73 mg, 139.40 μmol) was added. The reaction mixture was stirred at RT for 16 h. The solvent was removed under reduced pressure, and the residue was purified by reverse phase prep HPLC [Purification method: Column: X-bridge, C18 (150×19) mm, 5 micron; Mobile phase A: 0.1% TFA in water and Mobile phase B: MeCN] to obtain 7-benzyloxy-N-[2-[4-[1-(2,6-dioxo-3-piperidyl)-3-methyl-2-oxo-benzimidazol-5-yl]-1-piperidyl]ethyl]-5-fluoro-6-(1,1,4-trioxo-1,2,5-thiadiazolidin-2-yl)naphthalene-2-carboxamide (6, 25 mg, 27.42 μmol, 30% yield, TFA salt) as an off-white solid. LCMS (ES+): m/z 798.2 [M+H]⁺

### Step 4: N-[2-[4-[1-(2,6-dioxo-3-piperidyl)-3-methyl-2-oxo-benzimidazol-5-yl]-1-piperidyl]ethyl]-5-fluoro-7-hydroxy-6-(1,1,4-trioxo-1,2,5-thiadiazolidin-2-yl)naphthalene-2-carboxamide (Example 29)

Into a 10 mL single neck round bottom flask containing a well-stirred solution of 7-benzyloxy-N-[2-[4-[1-(2,6-dioxo-3-piperidyl)-3-methyl-2-oxo-benzimidazol-5-yl]-1-piperidyl]ethyl]-5-fluoro-6-(1,1,4-trioxo-1,2,5-thiadiazolidin-2-yl)naphthalene-2-carboxamide (6, 25 mg, 31.33 μmol) in toluene (1.5 mL) and DCM (1.5 mL), was added pentamethylbenzene (23.23 mg, 156.67 μmol. 25.33 μL) and the reaction mixture was cooled to −78° C. A solution of boron trichloride (1M in dichloromethane) (73.43 mg, 626.68 μmol, 630 μL) was added dropwise. After complete addition, the reaction was stirred at RT for 4 h. The reaction was quenched with 10% MeOH in DCM (2 mL). The reaction was concentrated under reduced pressure and the residue was purified by reverse phase prep HPLC [Purification method: Column: X-bridge, C18 (150×19 mm), 5 micron; Mobile phase A: 0.1% TFA in water and Mobile phase B: MeCN] to afford N-[2-[4-[1-(2,6-dioxo-3-piperidyl)-3-methyl-2-oxo-benzimidazol-5-yl]-1-piperidyl]ethyl]-5-fluoro-7-hydroxy-6-(1,1,4-trioxo-1,2,5-thiadiazolidin-2-yl)naphthalene-2-carboxamide (Example 29, 4.3 mg, 5.06 μmol, 16% yield, TFA salt) as an off-white solid. LCMS (ES+): m/z 708.1 [M+H]^{+ 1}H NMR (400 MHz, DMSO-d₆) δ 11.10 (s, 1H), 10.13 (s, 1H), 8.85 (s, 1H), 8.27 (s, 1H), 7.99 (d, J=8.7 Hz, 1H), 7.80 (dd, J=8.8, 1.6 Hz, 1H), 7.19 (s, 1H), 7.11-7.02 (m, 2H), 6.93 (d, J=8.2 Hz, 1H), 5.35 (dd, J=12.8, 5.4 Hz, 1H), 4.11 (s, 2H), 3.65 (s, 4H), 2.97-2.78 (m, 3H), 2.77-2.58 (m, 4H), 2.43 (s, 2H), 2.04-1.78 (m, 5H).

Examples 30-71 (structures shown in Table 1) were synthesized using methods similar to those described for Examples 1-29.

### Additional Intermediate Compounds

### 5-(1-fluoro-3-hydroxy-7-pyrrolidin-3-yl-2-naphthyl)-1,1-dioxo-1,2,5-thiadiazolidin-3-one (5)

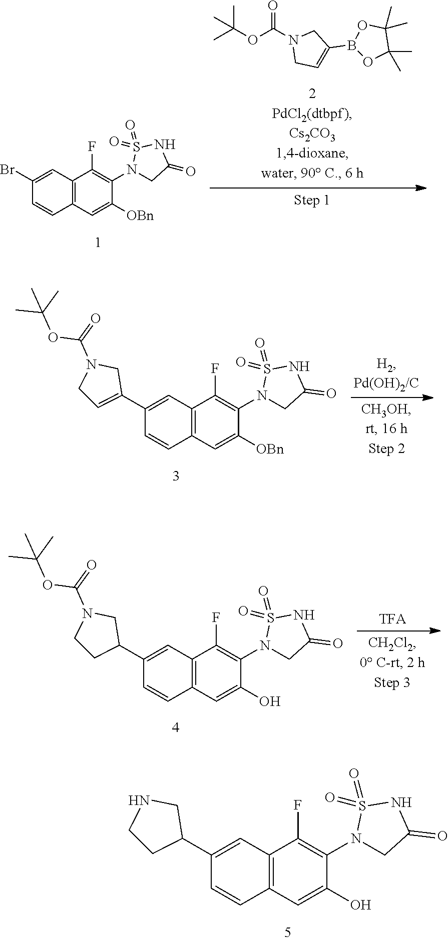 [SMILES: CC(C)(C)OC(=O)N1CC=C(B2OC(C)(C)C(C)(C)O2)C1 ; O=C1CN(c2c(OCc3ccccc3)cc3ccc(Br)cc3c2F)S(=O)(=O)N1 ; CC(C)(C)OC(=O)N1CC=C(c2ccc3cc(OCc4ccccc4)c(N4CC(=O)NS4(=O)=O)c(F)c3c2)C1 ; CC(C)(C)OC(=O)N1CCC(c2ccc3cc(O)c(N4CC(=O)NS4(=O)=O)c(F)c3c2)C1 ; O=C1CN(c2c(O)cc3ccc(C4CCNC4)cc3c2F)S(=O)(=O)N1]

### Step 1: tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3)

Into a 50 mL sealed tube containing a well-stirred solution of 5-(3-benzyloxy-7-bromo-1-fluoro-2-naphthyl)-1,1-dioxo-1,2,5-thiadiazolidin-3-one (1, 600 mg, 1.29 mmol) and tert-butyl 3-(4,4,5,5-tetramethyl-1,3,2-dioxaborolan-2-yl)-2,5-dihydropyrrole-1-carboxylate (2, 380.64 mg, 1.29 mmol) in anhydrous 1,4-dioxane (4.8 mL) and water (1.2 mL) were added Cs₂CO₃ (1.26 g, 3.87 mmol) at RT under nitrogen. The reaction mixture was degassed with nitrogen for 5 min. [1,1′-bis(di-tert-butylphosphino)ferrocene]dichloropalladium(II) (PdCl₂(dtbpf)) (42.02 mg, 64.48 μmol) was added and the resulting suspension was heated at 90° C. for 6 h. The reaction mixture was cooled to room temperature (RT) and poured into water (50 mL). The aqueous layer was extracted with ethyl acetate (EtOAc, 2×150 mL). The combined organic layer was dried over anhydrous sodium sulfate, filtered and concentrated under reduced pressure. The residue was triturated with diethyl ether (40 mL), filtered and dried to afford tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3, 400 mg, 637.28 μmol, 49% yield) as a brown solid. LCMS (ES+): m/z 454.1 [M-Boc+H]⁺.

### Step 2: tert-butyl 3-[8-fluoro-6-hydroxy-7-(1,1,4-trioxo-1,2,5-thiadiazolidin-2-yl)-2-naphthyl]pyrrolidine-1-carboxylate (4)

A 50 mL single neck round bottom flask containing a well-stirred solution of tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3, 1.2 g, 2.17 mmol) in methanol (50 mL) was purged with nitrogen for 2 min. Pd(OH)₂/C (304.41 mg, 2.17 mmol) was added and the mixture stirred under hydrogen for 16 h. The mixture was filtered through a pad of Celite and washed with methanol (20 mL). Concentration under reduced pressure afforded tert-butyl 3-[8-fluoro-6-hydroxy-7-(1,1,4-trioxo-1,2,5-thiadiazolidin-2-yl)-2-naphthyl]pyrrolidine-1-carboxylate (4, 951 mg, 1.80 mmol, 83% yield) as a brown solid. LCMS (ES+): m/z 410.0 [M-tBu+H]⁺.

### Step 3: 5-(1-fluoro-3-hydroxy-7-pyrrolidin-3-yl-2-naphthyl)-1,1-dioxo-1,2,5-thiadiazolidin-3-one (5)

Into a 50 mL single neck, round bottom flask containing a well-stirred solution of tert-butyl 3-[8-fluoro-6-hydroxy-7-(1,1,4-trioxo-1,2,5-thiadiazolidin-2-yl)-2-naphthyl]pyrrolidine-1-carboxylate (4, 900 mg, 1.93 mmol) in dichloromethane (DCM, 10 mL) under nitrogen at 0° C. was added TFA (220.46 mg, 1.93 mmol, 148.96 μL). After 2 h, the reaction mixture was evaporated to dryness and triturated with Et₂O to obtain 5-(1-fluoro-3-hydroxy-7-pyrrolidin-3-yl-2-naphthyl)-1,1-dioxo-1,2,5-thiadiazolidin-3-one (5, 900 mg, 1.82 mmol, 94% yield, TFA salt) as a white solid. LCMS (ES+): m/z 366.0 [M+H]⁺

### 2-[4-[5-fluoro-7-hydroxy-6-(1,1,4-trioxo-1,2,5-thiadiazolidin-2-yl)-2-naphthyl]pyrazol-1-yl]acetic acid (5)

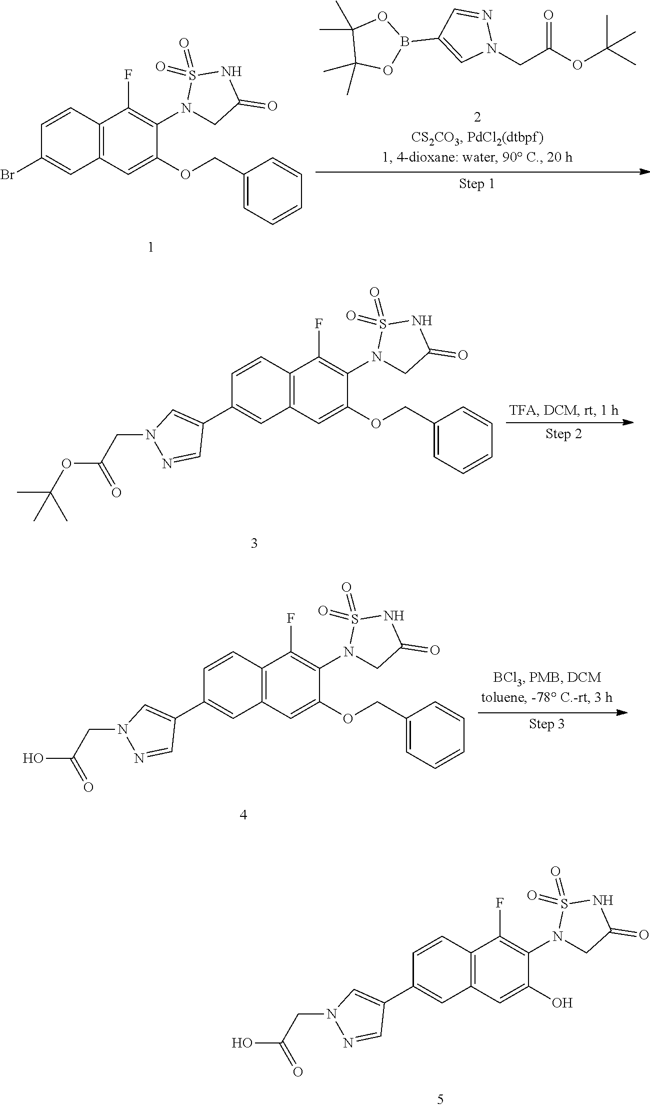 [SMILES: O=C1CN(c2c(OCc3ccccc3)cc3cc(Br)ccc3c2F)S(=O)(=O)N1 ; CC(C)(C)OC(=O)Cn1cc(B2OC(C)(C)C(C)(C)O2)cn1 ; CC(C)(C)OC(=O)Cn1cc(-c2ccc3c(F)c(N4CC(=O)NS4(=O)=O)c(OCc4ccccc4)cc3c2)cn1 ; O=C(O)Cn1cc(-c2ccc3c(F)c(N4CC(=O)NS4(=O)=O)c(OCc4ccccc4)cc3c2)cn1 ; O=C(O)Cn1cc(-c2ccc3c(F)c(N4CC(=O)NS4(=O)=O)c(O)cc3c2)cn1]

### Step 1: tert-butyl 2-[4-[7-benzyloxy-5-fluoro-6-(1,1,4-trioxo-1,2,5-thiadiazolidin-2-yl)-2-naphthyl]pyrazol-1-yl]acetate (3)

Into a 50 mL pressure tube containing a well-stirred solution of tert-butyl 2-[4-(4,4,5,5-tetramethyl-1,3,2-dioxaborolan-2-yl)pyrazol-1-yl]acetate (2, 582.91 mg, 1.72 mmol) and 5-(3-benzyloxy-6-bromo-1-fluoro-2-naphthyl)-1,1-dioxo-1,2,5-thiadiazolidin-3-one (1, 800 mg, 1.72 mmol) in water (3 mL) and 1,4-dioxane (3 mL) was added cesium carbonate (1.68 g, 5.16 mmol). The reaction mixture was degassed with nitrogen for 10 min. PdCl₂(dtbpf) (56.03 mg, 85.97 μmol) was added and the reaction mixture was heated at 90° C. After 20 h, the reaction mixture was cooled to RT, filtered through Celite, washing with EtOAc (30 mL). The filtrate was washed with water (15 mL). The aqueous layer was extracted with EtOAc (2×50 mL). The combined organic layer was washed with brine (30 mL), dried over anhydrous sodium sulfate, filtered and concentrated under reduced pressure. The residue was purified by flash silica gel column chromatography (10-15% MeOH in DCM) to afford tert-butyl 2-[4-[7-benzyloxy-5-fluoro-6-(1,1,4-trioxo-1,2,5-thiadiazolidin-2-yl)-2-naphthyl]pyrazol-1-yl]acetate (3, 850 mg, 1.26 mmol, 73% yield) as a brown solid. LCMS (ES+): m/z 567.2 [M+H]⁺.

### Step 2: 2-[4-[7-benzyloxy-5-fluoro-6-(1,1,4-trioxo-1,2,5-thiadiazolidin-2-yl)-2-naphthyl]pyrazol-1-yl]acetic acid (4)

Into a 25 mL round bottom flask containing a well-stirred solution of tert-butyl 2-[4-[7-benzyloxy-5-fluoro-6-(1,1,4-trioxo-1,2,5-thiadiazolidin-2-yl)-2-naphthyl]pyrazol-1-yl]acetate (3, 350 mg, 518.33 μmol) in DCM (3 mL) was added TFA (591.01 mg, 5.18 mmol, 399.33 μL) dropwise at 0° C. The reaction mixture was stirred at RT for 1 h. The volatiles were removed under reduced pressure and the residue triturated with diethyl ether (2×10 mL), filtered and dried to afford 2-[4-[7-benzyloxy-5-fluoro-6-(1,1,4-trioxo-1,2,5-thiadiazolidin-2-yl)-2-naphthyl]pyrazol-1-yl]acetic acid (4, 200 mg, 340.18 μmol, 66% yield) as a brown solid. LCMS (ES+): m/z 511.2 [M+H]⁺.

### Step 3: 2-[4-[5-fluoro-7-hydroxy-6-(1,1,4-trioxo-1,2,5-thiadiazolidin-2-yl)-2-naphthyl]pyrazol-1-yl]acetic acid (5)

Into a 25 mL round bottom flask containing a well-stirred solution of 2-[4-[7-benzyloxy-5-fluoro-6-(1,1,4-trioxo-1,2,5-thiadiazolidin-2-yl)-2-naphthyl]pyrazol-1-yl]acetic acid (4, 0.3 g, 510.27 μmol) and pentamethylbenzene (453.89 mg, 3.06 mmol, 494.97 μL) in a mixture of DCM (5 mL) and toluene (5 mL) was added boron trichloride (BCl₃) solution (1.0 M in DCM) (1.20 g, 10.21 mmol, 10.21 mL) under nitrogen at −78° C. The reaction mixture was stirred at RT. After 3 h, the reaction was cooled to −78° C. and quenched by dropwise addition of 10% MeOH in DCM (5 mL). The volatiles were removed under reduced pressure and the residue purified by reverse phase column chromatography [Purification method: Biotage C18 Column: Mobile phase A: 0.1% TFA in water; Mobile phase B: acetonitrile (MeCN)] to afford 2-[4-[5-fluoro-7-hydroxy-6-(1,1,4-trioxo-1,2,5-thiadiazolidin-2-yl)-2-naphthyl]pyrazol-1-yl]acetic acid (5, 210 mg, 455.90 μmol, 89% yield) as a brown solid. LCMS (ES+): m/z 421.0 [M+H]⁺.

### 5-(7-(azetidin-3-yloxy)-3-(benzyloxy)-1-fluoronaphthalen-2-yl)-1,2,5-thiadiazolidin-3-one 1,1-dioxide hydrochloride (4)

### Step 1: tert-butyl 3-((6-(benzyloxy)-7-(1,1-dioxido-4-oxo-1,2,5-thiadiazolidin-2-yl)-8-fluoronaphthalen-2-yl)oxy)azetidine-1-carboxylate (3)

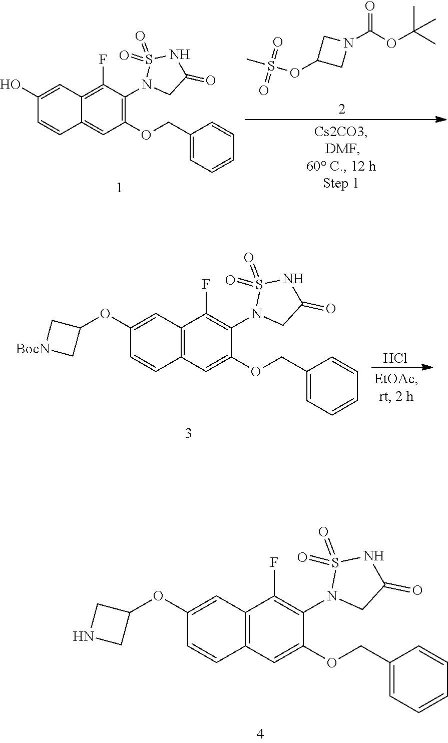 [SMILES: O=C1CN(c2c(OCc3ccccc3)cc3ccc(O)cc3c2F)S(=O)(=O)N1 ; CC(C)(C)OC(=O)N1CC(OS(C)(=O)=O)C1 ; CC(C)(C)OC(=O)N1CC(Oc2ccc3cc(OCc4ccccc4)c(N4CC(=O)NS4(=O)=O)c(F)c3c2)C1 ; Cl ; O=C1CN(c2c(OCc3ccccc3)cc3ccc(OC4CNC4)cc3c2F)S(=O)(=O)N1]

To a mixture of 5-(3-(benzyloxy)-1-fluoro-7-hydroxynaphthalen-2-yl)-1,2,5-thiadiazolidin-3-one 1,1-dioxide (1, 3.35 g, 5.96 mmol) and Cs₂CO₃ (4.92 g, 15.1 mmol) in DMF (20 mL) was added tert-butyl 3-((methylsulfonyl)oxy)azetidine-1-carboxylate (2, 5.70 g, 22.7 mmol) and the mixture heated to 60° C. and stirred for 12 h. The reaction mixture was adjusted to pH 5 with 1 M HCl, diluted with water (50 mL) and extracted with EtOAc (3×30 mL). The combined organic layers were washed with brine (2×30 mL), dried over sodium sulfate, filtered and concentrated under reduced pressure. The residue was purified by silica gel chromatography (elution with 0% to 10% MeOH: EtOAc) to afford tert-butyl 3-((6-(benzyloxy)-7-(1,1-dioxido-4-oxo-1,2,5-thiadiazolidin-2-yl)-8-fluoronaphthalen-2-yl)oxy)azetidine-1-carboxylate (3, 1.5 g, 2.4 mmol, 40% yield) as yellow solid. LCMS (ES−): m/z 556.2 [M−H]^{- 1}H NMR (501 MHZ, DMSO-d₆) δ 7.85 (dd, J=9.1, 1.3 Hz, 1H), 7.55-7.48 (m, 2H), 7.42 (s, 1H), 7.38 (t, J=7.3 Hz, 2H), 7.36-7.30 (m, 1H), 7.26 (dd, J=8.9, 2.5 Hz, 1H), 7.06 (d, J=2.6 Hz, 1H), 5.24 (s, 2H), 5.17 (tt, J=6.4, 3.9 Hz, 1H), 4.43 (d, J=2.7 Hz, 2H), 4.37 (s, 2H), 3.85 (dd, J=10.0, 3.7 Hz, 2H), 1.40 (s, 9H).

### Step 2: 5-(7-(azetidin-3-yloxy)-3-(benzyloxy)-1-fluoronaphthalen-2-yl)-1,2,5-thiadiazolidin-3-one 1,1-dioxide hydrochloride (4)

A solution of tert-butyl 3-((6-(benzyloxy)-7-(1,1-dioxido-4-oxo-1,2,5-thiadiazolidin-2-yl)-8-fluoronaphthalen-2-yl)oxy)azetidine-1-carboxylate (3, 3.35 g, 6.02 mmol) in hydrogen chloride/EtOAc (20 mL, 3.03 mmol) was stirred for 2 h. The reaction mixture was concentrated under reduced pressure and the residue triturated with 20% methanol: EtOAc. The solid was filtered and dried to afford 5-(7-(azetidin-3-yloxy)-3-(benzyloxy)-1-fluoronaphthalen-2-yl)-1,2,5-thiadiazolidin-3-one 1,1-dioxide hydrochloride (4, 2.5 g, 5.06 mmol, 84% yield) as an off white solid. LCMS (ES−): m/z 456.1 [M−H]^{- 1}H NMR (400 MHZ, DMSO-d₆) δ 4.01-4.08 (m, 2H) 4.41 (s, 2H) 4.52 (br d, J=5.70 Hz, 2H) 5.25 (s, 2H) 5.26-5.30 (m, 1H) 7.10 (s, 1H) 7.29 (br d, J=8.77 Hz, 1H) 7.32-7.41 (m, 3H) 7.44 (s, 1H) 7.52 (br d, J=7.02 Hz, 2H) 7.87 (br d, J=9.21 Hz, 1H) 8.96-9.34 (m, 2H).

### 3-[4-[1-(2,6-dioxo-3-piperidyl)-3-methyl-2-oxo-benzimidazol-5-yl]-1-piperidyl]propanoic acid (4)

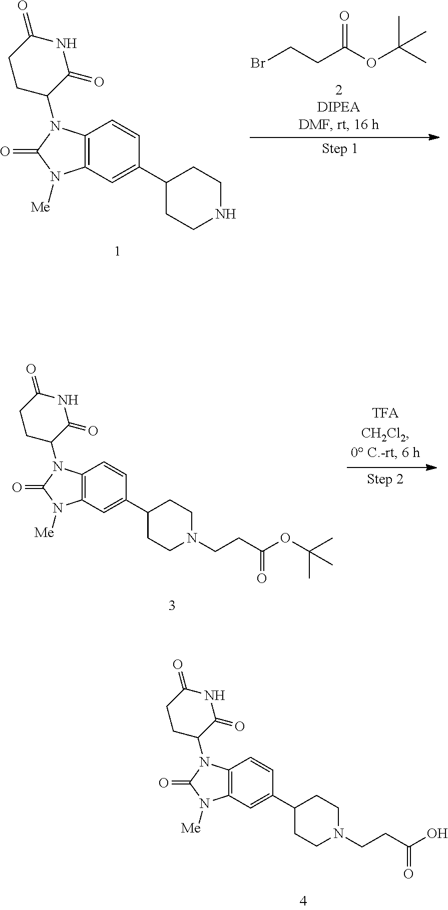

### Step 1: 3-[4-[1-(2,6-dioxo-3-piperidyl)-3-methyl-2-oxo-benzimidazol-5-yl]-1-piperidyl]propanoate (3)

Into a 50 mL single neck round bottom flask containing a well-stirred solution of 3-[3-methyl-2-oxo-5-(4-piperidyl)benzimidazol-1-yl]piperidine-2,6-dione (1, 500 mg, 1.32 mmol, HCl salt) in anhydrous DMF (10 mL) were added DIPEA (682.27 mg, 5.28 mmol, 919.50 μL) and tert-butyl 3-bromopropanoate (2, 413.91 mg, 1.98 mmol) at RT under nitrogen. After stirring for 16 h at RT, the reaction mixture was concentrated under reduced pressure and the residue was triturated with MTBE (2×25 mL), filtered and dried to afford tert-butyl 3-[4-[1-(2,6-dioxo-3-piperidyl)-3-methyl-2-oxo-benzimidazol-5-yl]-1-piperidyl]propanoate (3, 550 mg, 1.16 mmol, 88% yield) as an off-white solid. LCMS (ES+): m/z 471.2 [M+H]⁺.

### Step 2: 3-[4-[1-(2,6-dioxo-3-piperidyl)-3-methyl-2-oxo-benzimidazol-5-yl]-1-piperidyl]propanoic acid (4)

Into a 50 mL single neck round bottom flask containing a well-stirred solution of tert-butyl 3-[4-[1-(2,6-dioxo-3-piperidyl)-3-methyl-2-oxo-benzimidazol-5-yl]-1-piperidyl]propanoate (3, 550 mg, 1.17 mmol) was added TFA (1.33 g, 11.69 mmol, 900.49 μL) at 0° C. under nitrogen. The reaction was warmed to RT and stirred for 6 h. The reaction mixture was concentrated under reduced pressure and the residue was azeotroped with toluene (2×20 mL) and triturated with diethyl ether (2×25 mL) to afford 3-[4-[1-(2,6-dioxo-3-piperidyl)-3-methyl-2-oxo-benzimidazol-5-yl]-1-piperidyl]propanoic acid (4, 400 mg, 631.25 μmol, 54% yield, TFA salt). LCMS (ES−): hahaha

413.1 [M−H]⁻.

### 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetic acid (9)

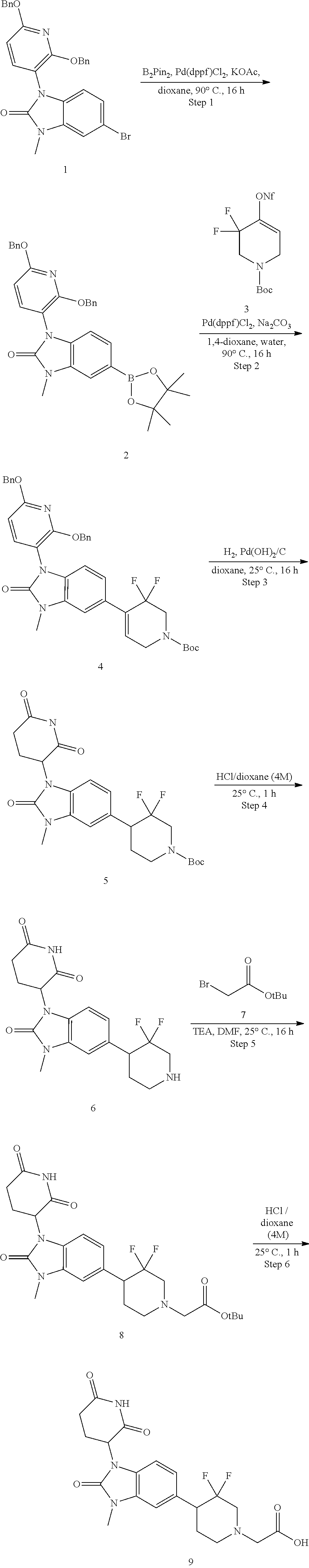

### Step 1: 1-(2,6-dibenzyloxy-3-pyridyl)-3-methyl-5-(4,4,5,5-tetramethyl-1,3,2-dioxaborolan-2-yl)benzimidazol-2-one (2)

To a solution of 1-(2,6-bis(benzyloxy)pyridin-3-yl)-5-bromo-3-methyl-1H-benzo[d]imidazol-2(3H)-one (1, 1.8 g, 3.49 mmol), potassium acetate (570) mg, 5.81 mmol) and 4,4,4′,4′,5,5,5′,5′-octamethyl-2,2′-bi (1,3,2-dioxaborolane) (1.77 g, 6.97 mmol) in dioxane (20 mL) was added [1,1′-bis(diphenylphosphino)ferrocene]dichloropalladium(II) (Pd(dppf)Cl₂, 127.53 mg, 174.29 μmol). The mixture was heated to 90° C. and stirred for 16 h under N₂. After cooling to RT, the mixture was concentrated under reduced pressure. The residue was diluted with EtOAc (30 mL) and water (30) mL). The layers were separated and the aqueous phase was extracted with EtOAc (3×30 mL). The organic layer was dried over sodium sulfate, filtered and concentrated under reduced pressure. The residue was purified by silica gel chromatography (petroleum ether/EtOAc=3/1) to afford 1-(2,6-dibenzyloxy-3-pyridyl)-3-methyl-5-(4,4,5,5-tetramethyl-1,3,2-dioxaborolan-2-yl)benzimidazol-2-one (2, 1.2 g, 2.02 mmol, 58% yield) as a yellow solid. ¹H NMR (400 MHZ, CDCl₃-d) δ=7.60 (d, J=8.4 Hz, 1H), 7.53 (d, J=7.6 Hz, 1H), 7.47 (s, 1H), 7.45-7.30 (m, 5H), 7.27-7.21 (m, 5H), 6.72 (d, J=7.6 Hz, 1H), 6.51 (d, J=8.4 Hz, 1H), 5.42 (s, 1H), 5.36-5.22 (m, 3H), 3.50) (s, 3H), 1.38 (s, 12H).

### Step 2: tert-butyl 4-[1-(2,6-dibenzyloxy-3-pyridyl)-3-methyl-2-oxo-benzimidazol-5-yl]-3,3-difluoro-2,6-dihydropyridine-1-carboxylate (4)

To a solution of 1-(2,6-dibenzyloxy-3-pyridyl)-3-methyl-5-(4,4,5,5-tetramethyl-1,3,2-dioxaborolan-2-yl)benzimidazol-2-one (2, 1.2 g, 2.66 mmol), tert-butyl 3,3-difluoro-4-(1,1,2,2,3,3,4,4,4-nonafluorobutylsulfonyloxy)-2,6-dihydropyridine-1-carboxylate (3, 1.8 g, 3.48 mmol) and sodium carbonate (2 M in water, 4.00 mL) in dioxane (20 mL) was added Pd(dppf)Cl₂ (98 mg, 133.93 μmol). The mixture was stirred at 90° C. for 16 h under N₂. The reaction mixture was filtered and concentrated under reduced pressure. The residue was purified by silica gel chromatography (petroleum ether/EtOAc=5/1 to 2/1) to afford tert-butyl 4-[1-(2,6-dibenzyloxy-3-pyridyl)-3-methyl-2-oxo-benzimidazol-5-yl]-3,3-difluoro-2,6-dihydropyridine-1-carboxylate (4, 1.9 g, 2.61 mmol, 98% yield) as an pink oil. ¹H NMR (400 MHZ, CDCl₃-d) δ=7.60 (d, J=8.4 Hz, 1H), 7.47-7.33 (m, 5H), 7.25-7.18 (m, 5H), 7.14-7.07 (m, 2H), 6.67 (d, J=8.0 Hz, 1H), 6.51 (d, J=8.4 Hz, 1H), 6.33-6.15 (m, 1H), 5.53-5.41 (m, 1H), 5.35 (s, 2H), 5.31-5.21 (m, 1H), 4.26-4.16 (m, 2H), 4.06 (t, J=6.8 Hz, 1H), 3.95 (t, J=11.2 Hz, 2H), 3.46 (s, 3H), (s, 9H).

### Step 3: tert-butyl 4-[1-(2,6-dioxo-3-piperidyl)-3-methyl-2-oxo-benzimidazol-5-yl]-3,3-difluoro-piperidine-1-carboxylate (5)

To a solution of tert-butyl 4-[1-(2,6-dibenzyloxy-3-pyridyl)-3-methyl-2-oxo-benzimidazol-5-yl]-3,3-difluoro-2,6-dihydropyridine-1-carboxylate (4, 1.9 g, 2.90 mmol) in dioxane (50 mL) were added Pd(OH)₂/C (950 mg, 20% purity). The mixture was stirred at 25° C. under H₂ atmosphere (15 psi) for 16 h. The mixture was filtered and concentrated under reduced pressure. The residue was diluted with DMF (20 mL). EtOAc (50 mL) and water (50 mL). The layers were separated and the aqueous phase was extracted with EtOAc (3×50 mL). The organic layer was dried over sodium sulfate, filtered and concentrated in vacuo to afford tert-butyl 4-[1-(2,6-dioxo-3-piperidyl)-3-methyl-2-oxo-benzimidazol-5-yl]-3,3-difluoro-piperidine-1-carboxylate (5, 910 mg, 1.79 mmol, 61% yield) as an off-white solid. LCMS (ESI): m/z 479.3 [M+H]⁺.

### Step 4: 3-[5-(3,3-difluoro-4-piperidyl)-3-methyl-2-oxo-benzimidazol-1-yl]piperidine-2,6-dione (6)

A mixture of tert-butyl 4-[1-(2,6-dioxo-3-piperidyl)-3-methyl-2-oxo-benzimidazol-5-yl]-3,3-difluoro-piperidine-1-carboxylate (5, 900 mg, 1.88 mmol) in HCl (4 M in dioxane, 5 mL) was stirred at 25° C. for 1 h. The mixture was concentrated under reduced pressure to give crude product. The crude product was triturated with EtOAc (5 mL) at 25° C. for 10 min to afford 3-[5-(3,3-difluoro-4-piperidyl)-3-methyl-2-oxo-benzimidazol-1-yl]piperidine-2,6-dione (6, 800 mg, 1.74 mmol, 92% yield, HCl salt) as a yellow solid. LC-MC (ESI): m/z 379.0 [M+H]⁺.

### Step 5: tert-butyl 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetate (8)

To a solution of 3-[5-(3,3-difluoro-4-piperidyl)-3-methyl-2-oxo-benzimidazol-1-yl]piperidine-2,6-dione (6, 800 mg, 2.11 mmol) in DMF (10 mL) was added triethylamine (1.06 g, 10.52 mmol. 1.47 mL) at (° C.) The mixture was stirred at 25° C. for 15 min. tert-Butyl 2-bromoacetate (7, 621 mg, 3.18 mmol) was added and stirred at 25° C. for 16 h. The mixture was concentrated and the residue was diluted with EtOAc (50 mL) and water (20 mL). The layers were separated and the aqueous phase was extracted with EtOAc (3×20 mL). The organic layer was dried over sodium sulfate, filtered and concentrated under reduced pressure. The residue was purified by silica gel chromatography (petroleum ether/EtOAc=3/1 to 1/0) to afford tert-butyl 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetate (8, 900 mg, 1.72 mmol, 81% yield) as a yellow solid. LCMS (ESI): m/z 493.2 [M+H]⁺.

### Step 6: 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetic acid (9)

A mixture of tert-butyl 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetate (8, 900 mg, 1.83 mmol) in HCl (4 M in dioxane, 456.84 μL) was stirred at 25° C. for 1 h. The mixture was concentrated under reduced pressure to give 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetic acid (9, 800 mg, 1.61 mmol, 88% yield, HCl salt) as a yellow solid. LCMS (ESI): m/z 437.2 [M+H]⁺.

### 3-[5-[4-(aminomethyl)cyclohexyl]-3-methyl-2-oxo-benzimidazol-1-yl]piperidine-2,6-dione (6a) and 3-[5-[4-(aminomethyl)cyclohexyl]-3-methyl-2-oxo-benzimidazol-1-yl]piperidine-2,6-dione (6b)

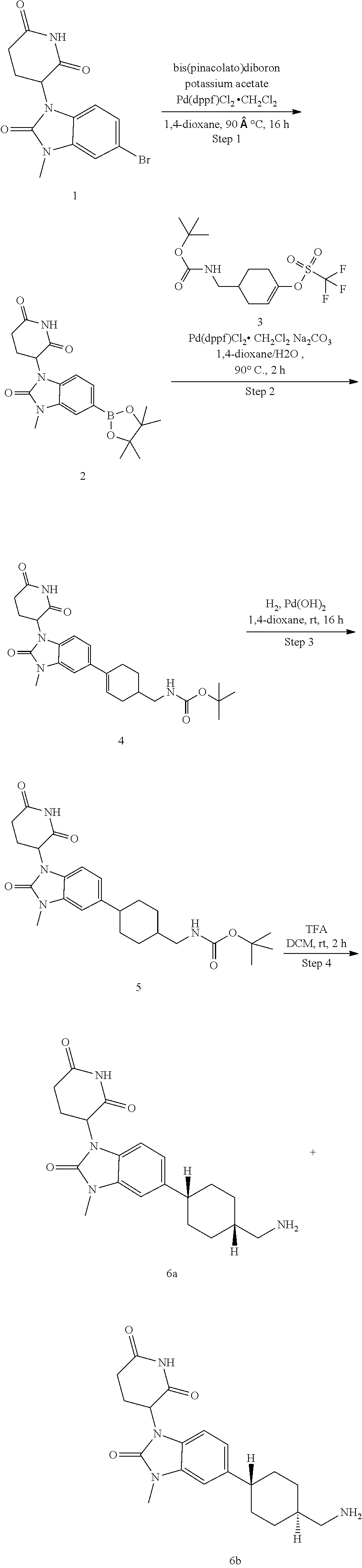

### Step 1: 3-[3-methyl-2-oxo-5-(4,4,5,5-tetramethyl-1,3,2-dioxaborolan-2-yl)benzimidazol-1-yl]piperidine-2,6-dione (2)

Into a 10 mL sealed tube containing a well-stirred solution of 3-(5-bromo-3-methyl-2-oxo-benzimidazol-1-yl)piperidine-2,6-dione (1, 100 mg, 295.72 μmol) in 1,4-Dioxane (2 mL) were added Bis(pinacolato) diboron (120.98 mg, 476.40 μmol) at RT under nitrogen. The reaction mixture was degassed with nitrogen for 5 min. Pd(dppf)Cl₂·CH₂Cl₂ (24.15 mg, 29.57 μmol) was added and the resulting suspension was heated at 90° C. for 16 h. The reaction mixture was cooled to RT, filtered through a pad of Celite, washing with MeCN (25 mL). The filtrate was concentrated under reduced pressure and the residue was purified by flash silica-gel (230-400) mesh) column chromatography (0-100% EtOAc/petroleum ether) to afford 3-[3-methyl-2-oxo-5-(4,4,5,5-tetramethyl-1,3,2-dioxaborolan-2-yl)benzimidazol-1-yl]piperidine-2,6-dione (2, 100 mg, 222.50) μmol, 75% yield) as an off white solid. LCMS (ES+): m/z 386.2 [M+H]⁺.

### Step 2: tert-butyl N-[[4-[1-(2,6-dioxo-3-piperidyl)-3-methyl-2-oxo-benzimidazol-5-yl]cyclohex-3-en-1-yl]methyl]carbamate (4)

Into a 50 mL pressure tube containing a well-stirred solution of 3-[3-methyl-2-oxo-5-(4,4,5,5-tetramethyl-1,3,2-dioxaborolan-2-yl)benzimidazol-1-yl]piperidine-2,6-dione (2, 1.12 g, 2.60) mmol) and [4-[(tert-butoxycarbonylamino)methyl]cyclohexen-1-yl] trifluoromethanesulfonate (3, 1.14 g, 2.86 mmol) in 1,4-dioxane (10 mL) and water (1 mL) was added sodium carbonate (825.42 mg, 7.79 mmol) at RT under nitrogen. The resulting mixture was degassed with nitrogen for 5 minutes. Pd(dppf)Cl₂·CH₂Cl₂ (317.99 mg, 389.39 μmol) was added and the mixture purged with nitrogen for 5 min. The tube was sealed and heated to 90° C. for 2 h. The reaction mixture was cooled and quenched with water (150 mL) and extracted with EtOAc (2×200 mL). The organic layer was dried over anhydrous sodium sulfate, filtered and concentrated under reduced pressure. The residue was purified by reverse phase column chromatography [Purification method: Siliasep premium C-18, 25 μm, 120 g, Mobile phase A: 0.1% TFA in water, Mobile phase B: MeCN] to afford tert-butyl N-[[4-[1-(2,6-dioxo-3-piperidyl)-3-methyl-2-oxo-benzimidazol-5-yl]cyclohex-3-en-1-yl]methyl]carbamate (4, 520 mg, 1.00 mmol, 39% yield) as an off-white solid. LCMS (ES+): m/z 369.2 [M-Boc+H]⁺.

### Step 3: tert-butyl N-[[4-[1-(2,6-dioxo-3-piperidyl)-3-methyl-2-oxo-benzimidazol-5-yl]cyclohexyl]methyl]carbamate (5)

Into a 100 mL single neck round bottom flask containing a well-stirred solution of tert-butyl N-[[4-[1-(2,6-dioxo-3-piperidyl)-3-methyl-2-oxo-benzimidazol-5-yl]cyclohex-3-en-1-yl]methyl]carbamate (4, 600 mg, 1.15 mmol) in 1,4-dioxane (15 mL) was added palladium hydroxide (20% on carbon) (600 mg, 0.85 mmol, 20% purity). The suspension was stirred under hydrogen atmosphere at RT. After 16 h, the reaction mixture was filtered through Celite, washed with 1,4-dioxane (100 mL) and concentrated under reduced pressure. The residue was triturated with diethyl ether (15 mL) to afford tert-butyl N-[[4-[1-(2,6-dioxo-3-piperidyl)-3-methyl-2-oxo-benzimidazol-5-yl]cyclohexyl]methyl]carbamate (5, 550 mg, 1.05 mmol, 91% yield) as a pale yellow solid. LCMS (ES+): m/z 415.2 [M-tBu+H]⁺.

### Step 4: 3-[5-[4-(aminomethyl)cyclohexyl]-3-methyl-2-oxo-benzimidazol-1-yl]piperidine-2,6-dione (6a and 6b)

Into a 50 mL single neck round bottom flask containing a well-stirred solution of tert-butyl N-[[4-[1-(2,6-dioxo-3-piperidyl)-3-methyl-2-oxo-benzimidazol-5-yl]cyclohexyl]methyl]carbamate (5, 500 mg, 956.31 μmol) in DCM (3 mL) was added trifluoroacetic acid (1.33 g, 11.68 mmol, 900.00 μL) at 0° C. After 2 h at RT, the reaction mixture was concentrated under reduced pressure and the residue was purified by reverse phase prep HPLC [Purification method: Column: X-Select C18 (19×150 mm), 5 micron; Mobile phase A: 0.1% TFA in water, Mobile phase B: MeCN] to afford 3-[5-[4-(aminomethyl)cyclohexyl]-3-methyl-2-oxo-benzimidazol-1-yl]piperidine-2,6-dione in two fractions: 6a (first eluted fraction, 120 mg, 247.42 μmol, 26% yield) as an off white solid and 6b (second eluted fraction, 150 mg, 304.70 μmol, 32% yield) as an off white solid. LCMS (ES+): m/z 371.3 [M+H]⁺. The relative stereochemistry of 6a and 6b were not determined.

### 2-[4-[1-(2,6-dioxo-3-piperidyl)-3-methyl-2-oxo-benzimidazol-5-yl]phenyl]acetic acid (3)

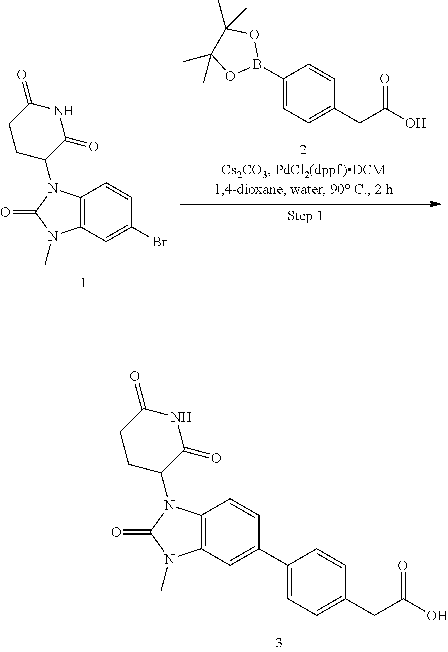 [SMILES: Cn1c(=O)n(C2CCC(=O)NC2=O)c2ccc(Br)cc21 ; CC1(C)OB(c2ccc(CC(=O)O)cc2)OC1(C)C ; Cn1c(=O)n(C2CCC(=O)NC2=O)c2ccc(-c3ccc(CC(=O)O)cc3)cc21]

### Step 1: 2-[4-[1-(2,6-dioxo-3-piperidyl)-3-methyl-2-oxo-benzimidazol-5-yl]phenyl]acetic acid (3)

Into a 25 mL pressure tube containing a well-stirred solution of 3-(5-bromo-3-methyl-2-oxo-benzimidazol-1-yl)piperidine-2,6-dione (1, 550 mg, 1.63 mmol) and 2-[4-(4,4,5,5-tetramethyl-1,3,2-dioxaborolan-2-yl)phenyl]acetic acid (2, 639.47 mg, 2.44 mmol) in 1,4-dioxane (7 mL) and water (0.75 mL) was added cesium carbonate (529.93 mg, 1.63 mmol). The mixture was degassed with nitrogen for 10 min. Pd(dppf)Cl₂·CH₂Cl₂ (199.23 mg, 243.97 μmol) was added. The tube was sealed and the mixture stirred at 90° C. for 16 h. The reaction mixture was filtered through Celite and washed with 1,4-dioxane (125 mL). The filtrate was concentrated under reduced pressure and the residue was purified by reverse phase column chromatography [Purification method: Siliasep C18 120 g, column; Mobile phase A: 0.1% formic acid in water; Mobile phase B: MeCN] to obtain 2-[4-[1-(2,6-dioxo-3-piperidyl)-3-methyl-2-oxo-benzimidazol-5-yl]phenyl]acetic acid (3, 250 mg, 603.72 μmol, 37% yield) as an off-white solid. LCMS (ES+): m/z 394.0 [M+H]⁺.

### 2-(1-(1-(2,6-dioxopiperidin-3-yl)-3-methyl-2-oxo-2,3-dihydro-1H-benzo[d]imidazol-5-yl)piperidin-4-yl)acetic acid (5)

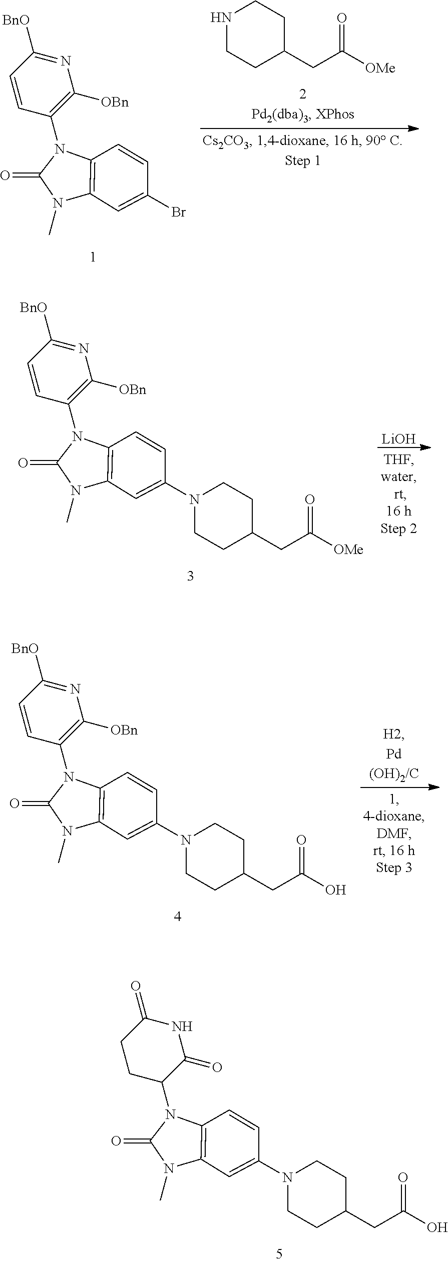 [SMILES: Cn1c(=O)n(-c2ccc(OCc3ccccc3)nc2OCc2ccccc2)c2ccc(Br)cc21 ; COC(=O)CC1CCNCC1 ; COC(=O)CC1CCN(c2ccc3c(c2)n(C)c(=O)n3-c2ccc(OCc3ccccc3)nc2OCc2ccccc2)CC1 ; "LiOH" ; Cn1c(=O)n(-c2ccc(OCc3ccccc3)nc2OCc2ccccc2)c2ccc(N3CCC(CC(=O)O)CC3)cc21 ; Cn1c(=O)n(C2CCC(=O)NC2=O)c2ccc(N3CCC(CC(=O)O)CC3)cc21]

### Step 1: Methyl 2-(1-(1-(2,6-bis(benzyloxy)pyridin-3-yl)-3-methyl-2-oxo-2,3-dihydro-1H-benzo[d]imidazol-5-yl)piperidin-4-yl)acetate (3)

Into a 50 mL sealed tube containing a well-stirred solution of 5-bromo-1-(2,6-dibenzyloxy-3-pyridyl)-3-methyl-benzimidazol-2-one (1, 2.0 g, 3.87 mmol) and methyl 2-(4-piperidyl)acetate (2, 791.55 mg, 5.04 mmol) in 1,4-dioxane (20 mL) was added Cs₂CO₃ (3.79 g, 11.62 mmol). The reaction mixture was degassed with nitrogen for 10 min, then Pd₂(dba)₃ (532.00 mg, 580.96 μmol) and XPhos (461.58 mg, 968.26 μmol) were added. The reaction mixture was heated to 90° C. for 16 h. The reaction mixture was cooled to RT, filtered through a pad of Celite and washed with EtOAc (50 mL). The solvent was removed under reduced pressure and the residue purified by silica gel column chromatography (60-120 mesh, 50 g; 40-60% EtOAc in petroleum ether) to afford Methyl 2-(1-(1-(2,6-bis(benzyloxy)pyridin-3-yl)-3-methyl-2-oxo-2,3-dihydro-1H-benzo[d]imidazol-5-yl)piperidin-4-yl)acetate (3, 1.2 g, 52% yield) as a brown gummy solid. LCMS (ES+): m/z 593.2 [M+H]⁺.

### Step 2: 2-(1-(1-(2,6-bis(benzyloxy)pyridin-3-yl)-3-methyl-2-oxo-2,3-dihydro-1H-benzo[d]imidazol-5-yl)piperidin-4-yl)acetic acid (4)

Into a 50 mL single neck, round bottom flask containing a well-stirred solution of Methyl 2-(1-(1-(2,6-bis(benzyloxy)pyridin-3-yl)-3-methyl-2-oxo-2,3-dihydro-1H-benzo[d]imidazol-5-yl)piperidin-4-yl)acetate (3, 1.2 g, 2.02 mmol) in THF (8 mL) and water (2 mL) was added lithium hydroxide monohydrate (508.21 mg, 12.11 mmol) at RT. The reaction mixture was stirred for 16 h. The volatiles were removed under reduced pressure and the residue was diluted with water (20 mL) and acidified with 1.5N HCl (10 mL). The solid obtained was filtered and triturated with Et₂O (20 mL) to afford 2-(1-(1-(2,6-bis(benzyloxy)pyridin-3-yl)-3-methyl-2-oxo-2,3-dihydro-1H-benzo[d]imidazol-5-yl)piperidin-4-yl)acetic acid (4, 900 mg, 76% yield) as an off-white solid. LCMS (ES+): m/z 579.2[M+H]⁺.

### Step 3: 2-(1-(1-(2,6-dioxopiperidin-3-yl)-3-methyl-2-oxo-2,3-dihydro-1H-benzo[d]imidazol-5-yl)piperidin-4-yl)acetic acid (5)

Into a 100 mL single neck round bottom flask containing a well-stirred solution of 2-(1-(1-(2,6-bis(benzyloxy)pyridin-3-yl)-3-methyl-2-oxo-2,3-dihydro-1H-benzo[d]imidazol-5-yl)piperidin-4-yl)acetic acid (4, 800 mg, 1.37 mmol) in anhydrous DMF (10.0 mL) and 1,4-dioxane (10.0 mL) was added palladium hydroxide on carbon (20 wt. % 50% water) (1.6 g, 11.39 mmol) at RT. The reaction mixture was stirred for 16 hours under an atmosphere of hydrogen. The reaction mixture was filtered through a pad of Celite and washed with 1,4-dioxane (50 mL). Volatiles were evaporated and the residue was triturated with diethyl ether (10 mL) to afford 2-[1-[1-(2,6-dioxo-3-piperidyl)-3-methyl-2-oxo-benzimidazol-5-yl]-4-piperidyl]acetic acid (5, 300 mg, 44% yield) as a pink solid. LCMS (ES+): m/z 401.3[M+H]⁺.

### 2-[4-[1-(2,6-dioxo-3-piperidyl)-3-methyl-2-oxo-benzimidazol-5-yl]cyclohexyl]acetic acid (5)

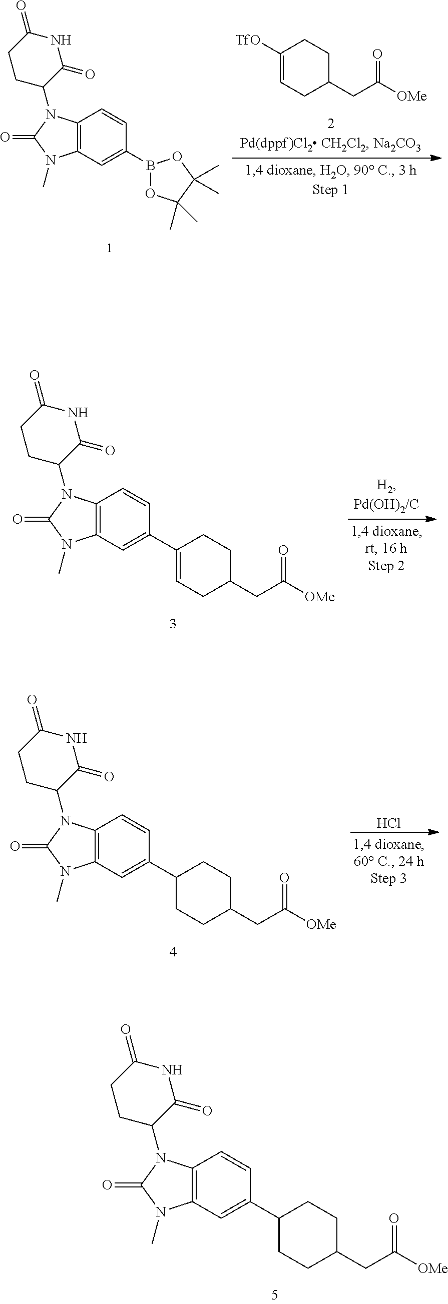 [SMILES: Cn1c(=O)n(C2CCC(=O)NC2=O)c2ccc(B3OC(C)(C)C(C)(C)O3)cc21 ; COC(=O)CC1CC=C(OS(=O)(=O)C(F)(F)F)CC1 ; COC(=O)CC1CC=C(c2ccc3c(c2)n(C)c(=O)n3C2CCC(=O)NC2=O)CC1 ; COC(=O)CC1CCC(c2ccc3c(c2)n(C)c(=O)n3C2CCC(=O)NC2=O)CC1 ; Cl ; COC(=O)CC1CCC(c2ccc3c(c2)n(C)c(=O)n3C2CCC(=O)NC2=O)CC1]

### Step 1: Methyl 2-[4-[1-(2,6-dioxo-3-piperidyl)-3-methyl-2-oxo-benzimidazol-5-yl]cyclohex-3-en-1-yl]acetate (3)

Into a 100 mL sealed tube containing a well-stirred solution of 3-[3-methyl-2-oxo-5-(4,4,5,5-tetramethyl-1,3,2-dioxaborolan-2-yl)benzimidazol-1-yl]piperidine-2,6-dione (1, 1.72 g, 4.05 mmol) and methyl 2-[4-(trifluoromethylsulfonyloxy)cyclohex-3-en-1-yl]acetate (2, 2.1 g, 6.60 mmol) in 1,4-dioxane (20 mL) and water (1.5 mL) was added Na₂CO₃ (2.10 g, 19.80 mmol) under nitrogen. The mixture was degassed with nitrogen for 5 min. Pd(dppf)Cl₂·CH₂Cl₂ (808.49 mg, 990.02 μmol) was added to the reaction mixture and heated to 90° C. for 3 h. The reaction mixture was cooled to RT, diluted with water (70 mL) and extracted with EtOAc (2×100 mL). The combined organic layers were dried over anhydrous Sodium sulfate, filtered and concentrated under reduced pressure. The residue was purified by flash column chromatography (100-200 mesh-100 g silica gel, Mobile phase: 70% EtOAc in Petroleum ether) to afford methyl 2-[4-[1-(2,6-dioxo-3-piperidyl)-3-methyl-2-oxo-benzimidazol-5-yl]cyclohex-3-en-1-yl]acetate (3, 800 mg, 1.19 mmol, 18% yield) as a pale yellow solid. LCMS (ES+): m/z 412.2 [M+H]⁺.

### Step 2: Methyl 2-[4-[1-(2,6-dioxo-3-piperidyl)-3-methyl-2-oxo-benzimidazol-5-yl]cyclohexyl]acetate (4)

Into a 50 mL single neck, round bottom flask containing a well-stirred solution of methyl 2-[4-[1-(2,6-dioxo-3-piperidyl)-3-methyl-2-oxo-benzimidazol-5-yl]cyclohex-3-en-1-yl]acetate (3, 200 mg, 297.48 μmol) in 1,4-dioxane (5 mL) was added Pd(OH)₂ on carbon (20 wt. %, 50% water) (122.41 mg, 871.63 μmol). The reaction was stirred for 16 h under an atmosphere of hydrogen. The reaction was filtered through a pad of Celite and washed with 1,4-dioxane (30 mL). The solvent was removed under reduced pressure and the residue triturated with diethyl ether to afford methyl 2-[4-[1-(2,6-dioxo-3-piperidyl)-3-methyl-2-oxo-benzimidazol-5-yl]cyclohexyl]acetate (4, 180 mg, 289.85 μmol, 97% yield) as an off-white solid. LCMS (ES+): m/z 414.2 [M+H]⁺.

### Step 3: 2-[4-[1-(2,6-dioxo-3-piperidyl)-3-methyl-2-oxo-benzimidazol-5-yl]cyclohexyl]acetic acid (5)

Into a 50 mL sealed tube containing a well-stirred solution of methyl 2-[4-[1-(2,6-dioxo-3-piperidyl)-3-methyl-2-oxo-benzimidazol-5-yl]cyclohexyl]acetate (4, 180 mg, 289.85 μmol) in 1,4-dioxane (4 mL) was added 1.5M HCl (289.85 μmol, 6 mL) under nitrogen. The resulting mixture was stirred at 60° C. for 24 h. The solvent was removed under reduced pressure and the residue was purified by reverse phase column chromatography [Column: Redisep HP gold-C18, 30 g; Mobile phase A: 0.1% TFA in water and Mobile phase B: MeCN] to afford 2-[4-[1-(2,6-dioxo-3-piperidyl)-3-methyl-2-oxo-benzimidazol-5-yl]cyclohexyl]acetic acid (5, 60 mg, 146.98 μmol, 51% yield) as an off-white solid. LCMS (ES+): m/z 400.0 [M+H]⁺.

### 2-[1-[1-(2,6-dioxo-3-piperidyl)-3-methyl-2-oxo-benzimidazol-5-yl]-4-hydroxy-4-piperidyl]acetic acid (5)

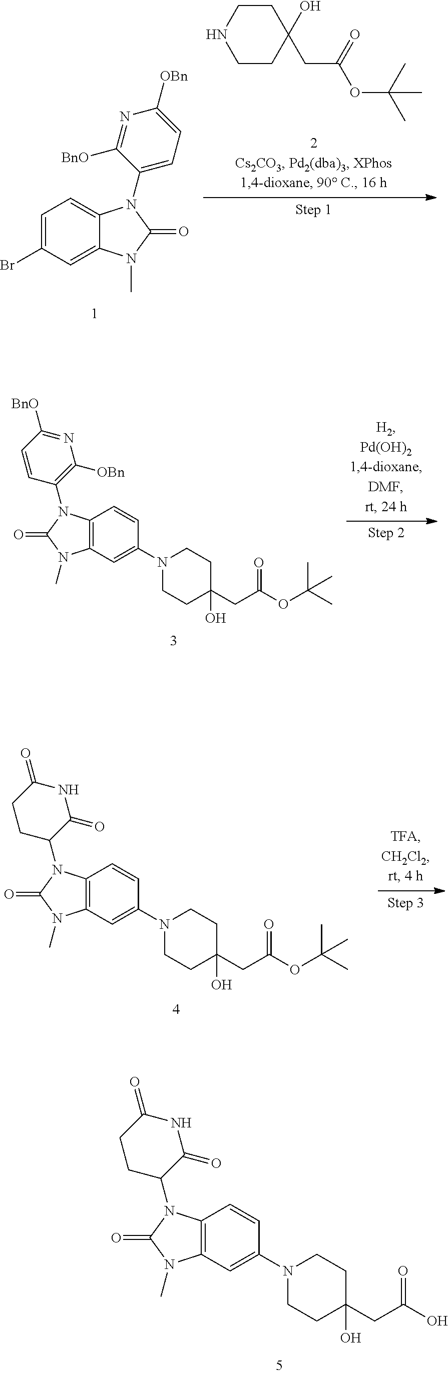 [SMILES: Cn1c(=O)n(-c2ccc(OCc3ccccc3)nc2OCc2ccccc2)c2ccc(Br)cc21 ; CC(C)(C)OC(=O)CC1(O)CCNCC1 ; Cn1c(=O)n(-c2ccc(OCc3ccccc3)nc2OCc2ccccc2)c2ccc(N3CCC(O)(CC(=O)OC(C)(C)C)CC3)cc21 ; Cn1c(=O)n(C2CCC(=O)NC2=O)c2ccc(N3CCC(O)(CC(=O)OC(C)(C)C)CC3)cc21 ; Cn1c(=O)n(C2CCC(=O)NC2=O)c2ccc(N3CCC(O)(CC(=O)O)CC3)cc21]

### Step 1: tert-butyl 2-[1-[1-(2,6-dibenzyloxy-3-pyridyl)-3-methyl-2-oxo-benzimidazol-5-yl]-4-hydroxy-4-piperidyl]acetate (3)

Into a 50 mL pressure tube containing a well-stirred solution of 5-bromo-1-(2,6-dibenzyloxy-3-pyridyl)-3-methyl-benzimidazol-2-one (1, 700 mg, 1.33 mmol) and tert-butyl 2-(4-hydroxy-4-piperidyl)acetate (2, 451.58 mg, 1.99 mmol) in anhydrous 1,4-dioxane (7 mL) was added cesium carbonate (1.30 g, 3.99 mmol). The mixture was degassed with nitrogen for 15 min. Then, tris(dibenzylideneacetone)dipalladium(0)) (145.98 mg, 159.42 μmol) and XPhos (126.66 mg. 265.69 μmol) were added. The vial was sealed and heated at 90° C. After 16 h, the reaction mixture was filtered through a pad of celite and washed with EtOAc (50 mL). The filtrate was concentrated under reduced pressure and the residue was purified by flash silica gel column chromatography (90% EtOAc in petroleum ether) to afford tert-butyl 2-[1-[1-(2,6-dibenzyloxy-3-pyridyl)-3-methyl-2-oxo-benzimidazol-5-yl]-4-hydroxy-4-piperidyl]acetate (3, 600 mg, 901.62 μmol, 68% yield) as pale yellow solid. LCMS (ES+): m/z 651.3 [M+H]⁺.

### Step 2: tert-butyl 2-[1-[1-(2,6-dioxo-3-piperidyl)-3-methyl-2-oxo-benzimidazol-5-yl]-4-hydroxy-4-piperidyl]acetate (4)

Into a 50 mL single neck round bottom flask containing a well-stirred solution of tert-butyl 2-[1-[1-(2,6-dibenzyloxy-3-pyridyl)-3-methyl-2-oxo-benzimidazol-5-yl]-4-hydroxy-4-piperidyl]acetate (3, 600 mg, 894.34 μmol) in anhydrous 1,4-dioxane (15 mL) was added palladium hydroxide on carbon (20 wt. %, 50% water) (356.33 mg, 507.46 μmol, 20% purity) at RT. The suspension was stirred under an atmosphere of hydrogen for 20 h. The reaction mixture was filtered through Celite and concentrated under reduced pressure. The residue was purified by reverse phase prep HPLC [Purification method: Column: X-Bridge C18 (150×19) 5 micron; Mobile phase A: 0.1% TFA in water; Mobile phase B: MeCN] to afford tert-butyl 2-[1-[1-(2,6-dioxo-3-piperidyl)-3-methyl-2-oxo-benzimidazol-5-yl]-4-hydroxy-4-piperidyl]acetate (4, 400 mg, 681.40 μmol, 76% yield, TFA salt) as a white solid. LCMS (ES+): m/z 473.2 [M+H]⁺.

### Step 3: 2-[1-[1-(2,6-dioxo-3-piperidyl)-3-methyl-2-oxo-benzimidazol-5-yl]-4-hydroxy-4-piperidyl]acetic acid (5)

Into a 50 mL single neck round bottom flask containing a well-stirred solution of tert-butyl 2-[1-[1-(2,6-dioxo-3-piperidyl)-3-methyl-2-oxo-benzimidazol-5-yl]-4-hydroxy-4-piperidyl]acetate (4, 380 mg, 788.09 μmol) in anhydrous DCM (5 mL) was added TFA (1.78 g, 15.58 mmol, 1.2 mL). After 4 h, the solvent was removed under reduced pressure and the residue was triturated with diethyl ether (10 mL), filtered and dried to afford 2-[1-[1-(2,6-dioxo-3-piperidyl)-3-methyl-2-oxo-benzimidazol-5-yl]-4-hydroxy-4-piperidyl]acetic acid (5, 400 mg, 650.24 μmol, 83% yield, TFA salt) as an off-white solid. LCMS (ES+): m/z 417.2 [M+H]⁺.

### 2-(1-(3-(2,6-dioxopiperidin-3-yl)-1-methyl-1H-indazol-6-yl)-4-hydroxypiperidin-4-yl)acetic acid (11)

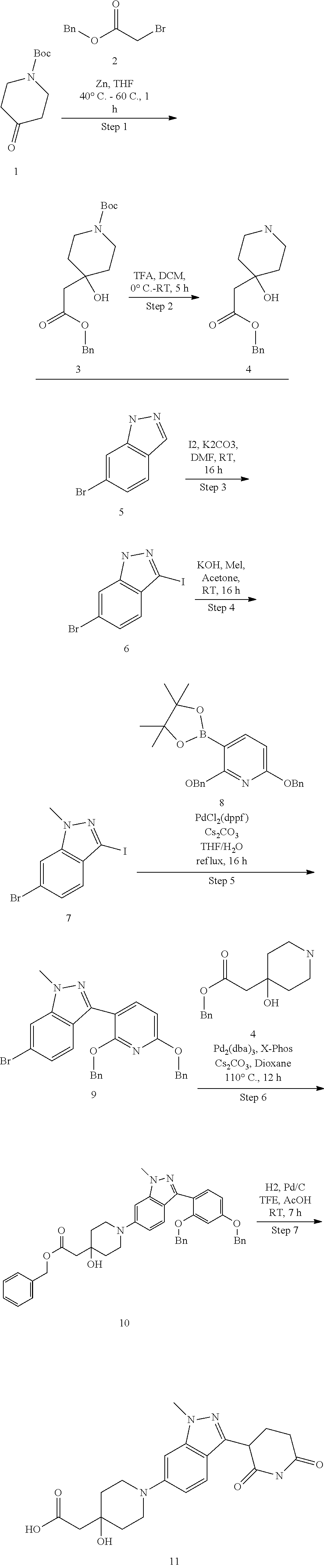

### Step 1: tert-butyl 4-(2-benzyloxy-2-oxo-ethyl)-4-hydroxy-piperidine-1-carboxylate (3)

To a stirred solution of benzyl 2-bromoacetate (2, 34.49 g, 150.57 mmol) in THF (200 mL) was added freshly activated zinc dust (16.41 g, 250.95 mmol) and heated at 40° C. for 20 min. The reaction mixture was allowed to cool to RT and tert-butyl 4-oxopiperidine-1-carboxylate (1, 20 g, 100.38 mmol) was added. The reaction mixture was heated at 60° C. for 1 h. The reaction mixture was cooled to RT, filtered through Celite, washing with THF and concentrated under reduced pressure. The residue was diluted with EtOAc, washed with water and brine, dried over anhydrous sodium sulfate, filtered and concentrated under reduced pressure. The residue was purified by column chromatography (15-20% EtOAc-hexane) to afford tert-butyl 4-(2-benzyloxy-2-oxo-ethyl)-4-hydroxy-piperidine-1-carboxylate (3, 15.4 g, 43.19 mmol, 43% yield) as a light yellow liquid. LCMS (ES+): m/z 350 [M+H]⁺.

### Step 2: benzyl 2-(4-hydroxy-4-piperidyl)acetate (4)

To a stirred solution of tert-butyl 4-(2-benzyloxy-2-oxo-ethyl)-4-hydroxy-piperidine-1-carboxylate (3, 17.8 g, 50.94 mmol) in DCM (250 mL), TFA (1.01 mol, 116.5 mL) was added dropwise at 0° C. The mixture was warmed to RT and stirred for 16 h. The reaction mixture was concentrated under reduced pressure, triturated with 20% EtOAc-hexane to afford benzyl 2-(4-hydroxy-4-piperidyl)acetate (4, 10 g, 34.99 mmol, 69% yield, TFA salt) as white solid. LCMS (ES+): m/z 250 [M+H]⁺.

### Step 3: 6-bromo-3-iodo-1H-indazole (6)

To a degassed stirred solution of 6-bromo-1H-indazole (5, 20 g, 101.51 mmol) in DMF (200 mL) was added potassium carbonate (42.09 g, 304.52 mmol) followed by solution of iodine (38.64 g, 152.26 mmol) in DMF (200 mL) dropwise over a period of 45 min. The reaction mixture was stirred at RT for 16 h. After completion of the reaction, reaction mixture was poured into ice-cooled saturated solution of sodium thiosulfate. The precipitate was filtered, thoroughly washed with water, pentane and dried to afford 6-bromo-3-iodo-1H-indazole (6, 23.3 g, 71.43 mmol, 70% yield) as an off white solid. LC MS: m/z 323 [M+H]⁺.

### Step 4: 6-bromo-3-iodo-1-methyl-1H-indazole (7)

To a stirred solution of 6-bromo-3-iodo-1H-indazole (6, 40 g, 123.87 mmol) in acetone (400 mL) was added potassium hydroxide (17.37 g, 309.67 mmol) followed by slow dropwise addition of methyl iodide (19.28 mL, 309.67 mmol) over a period of 30 min and stirred at RT for 16 h. The reaction mixture was filtered through Celite, washed with acetone and concentrated under reduced pressure. The residue was diluted with EtOAc, washed with water and brine, dried over anhydrous sodium sulfate, filtered and concentrated under reduced pressure. The compound was purified by column chromatography (15-20% EtOAc-hexane) to afford 6-bromo-3-iodo-1-methyl-indazole (7, 28 g, 82.27 mmol, 66% yield). LCMS: m/z 337 [M+H]⁺.

### Step 5: 3-(2,6-bis(benzyloxy)pyridin-3-yl)-6-bromo-1-methyl-1H-indazole (9)

To a stirred solution of 6-bromo-3-iodo-1-methyl-indazole (7, 15 g, 44.52 mmol) and 2,6-dibenzyloxy-3-(4,4,5,5-tetramethyl-1,3,2-dioxaborolan-2-yl)pyridine (8, 18.58 g, 44.52 mmol) in THF (450 mL) and water (75 mL), was added cesium carbonate (43.51 g, 133.55 mmol) and thoroughly purged with argon. Pd(dppf)Cl₂·CH₂Cl₂ (1.82 g, 2.23 mmol) was added under inert atmosphere. The resulting mixture was heated to reflux for 16 h. The reaction mixture was diluted with EtOAc, filtered through Celite and washed with EtOAc. Combined organic part was washed with water and brine, dried over anhydrous sodium sulfate, filtered and concentrated under reduced pressure. The residue was purified by column chromatography (5% EtOAc-hexane) to afford 6-bromo-3-(2,6-dibenzyloxy-3-pyridyl)-1-methyl-indazole (9, 11 g, 21.76 mmol, 49% yield) as off-white solid. LCMS: m/z 500 [M+H]⁺.

### Step 6: benzyl-2-(1-(3-(2,6-bis(benzyloxy)pyridin-3-yl)-1-methyl-1H-indazol-6-yl)-4-hydroxypiperidin-4-yl)acetate (10)

To a stirred solution of 6-bromo-3-(2,6-dibenzyloxy-3-pyridyl)-1-methyl-indazole (9, 5 g, 99.9 mmol) and benzyl 2-(4-hydroxy-4-piperidyl)acetate (4, 5.45 g, 14.99 mmol) in 1,4-Dioxane (30 mL), cesium carbonate (9.77 g, 29.98 mmol) was added. The resulting mixture was degassed with argon and Xphos (952.7 mg, 2.00 mmol), Pd₂(dba); (915.0 mg, 1.00 mmol) were added under inert atmosphere. The mixture was heated at 110° C. for 12 h. The reaction mixture was diluted with EtOAc, filtered through Celite and washed with EtOAc. Combined organic part was washed with water and brine, dried over anhydrous sodium sulfate, filtered and concentrated under reduced pressure. The residue was purified by column chromatography to afford benzyl-2-(1-(3-(2,6-bis(benzyloxy)pyridin-3-yl)-1-methyl-1H-indazol-6-yl)-4-hydroxypiperidin-4-yl)acetate (10, 2.6 g, 3.69 mmol, 37% yield). LCMS: m/z 669 [M+H]⁺.

### Step 7: 2-(1-(3-(2,6-dioxopiperidin-3-yl)-1-methyl-1H-indazol-6-yl)-4-hydroxypiperidin-4-yl)acetic acid (11)

To a degassed solution of benzyl-2-(1-(3-(2,6-bis(benzyloxy)pyridin-3-yl)-1-methyl-1H-indazol-6-yl)-4-hydroxypiperidin-4-yl)acetate (10, 2 g, 2.99 mmol) in 2,2,2-Trifluoroethanol (80 mL), 10% Palladium on carbon (50% wet, 2.00 g, 18.79 mmol) was added. The resulting mixture was stirred under hydrogen balloon pressure for 7 h. The reaction mixture was filtered through Celite, washed with ethanol, and concentrated under reduced pressure. The residue was purified by column chromatography to afford 2-(1-(3-(2,6-dioxopiperidin-3-yl)-1-methyl-1H-indazol-6-yl)-4-hydroxypiperidin-4-yl)acetic acid (11, 807 mg, 1.91 mmol, 64% yield). LCMS: m/z 401 [M+H]^{+ 1}H NMR (400 MHZ, DMSO-d₆) δ 10.85 (s, 1H), 7.46 (d, J=8.9 Hz, 1H), 6.90 (d, J=9.1 Hz, 1H), 6.84 (s, 1H), 6.08 (bs, 1H), 4.26-4.22 (m, 1H), 3.87 (s, 3H), 3.53-3.46 (m, 2H), 3.15-3.04 (m, 2H), 2.67-2.56 (m, 2H), 2.37 (s, 2H), 2.32-2.27 (m, 1H), 2.17-2.13 (m, 1H), 1.81-1.76 (m, 2H), 1.70-1.67 (m, 2H).

### 2-[[1-[1-(2,6-dioxo-3-piperidyl)-3-methyl-2-oxo-benzimidazol-5-yl]-4-piperidyl]oxy]acetic acid (4)

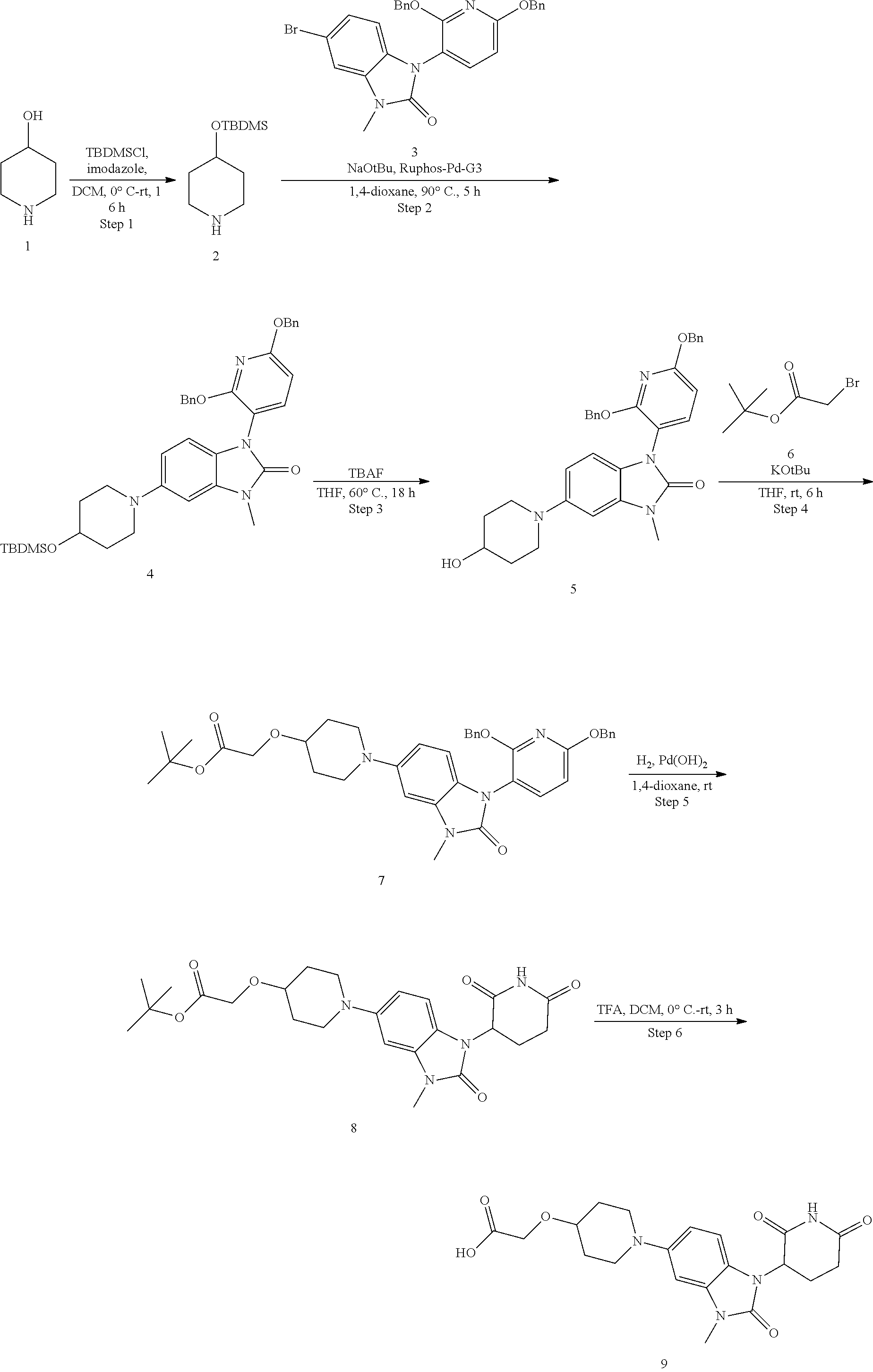

### Step 1: tert-butyl-dimethyl-(4-piperidyloxy)silane (2)

Into a 250 mL two neck round bottom flask containing a well-stirred suspension of piperidin-4-ol (1, 5 g, 49.43 mmol) in DCM (100 mL) was added imidazole (6.73 g, 98.87 mmol). The reaction mixture was cooled to 0° C. and tert-butyldimethylsilyl chloride (8.20 g, 54.38 mmol, 10.12 mL) was added. The reaction mixture was stirred at RT for 16 h. The reaction mixture was extracted with DCM (2×300 mL), washed with water (250 mL) and brine (200 mL). The combined organics were dried over sodium sulfate, filtered and the solvent removed under reduced pressure to afford tert-butyl-dimethyl-(4-piperidyloxy)silane (2, 4 g, 11.77 mmol, 24% yield) as a pale yellow liquid. LCMS (ES+): m/z 216.2 [M+H]⁺.

### Step 2: 5-[4-[tert-butyl(dimethyl)silyl]oxy-1-piperidyl]-1-(2,6-dibenzyloxy-3-pyridyl)-3-methyl-benzimidazol-2-one (4)

Into a 50 mL pressure tube containing a well-stirred suspension of 5-bromo-1-(2,6-dibenzyloxy-3-pyridyl)-3-methyl-benzimidazol-2-one (3, 800 mg, 1.55 mmol) in anhydrous 1,4-dioxane (15 mL) was added tert-butyl-dimethyl-(4-piperidyloxy)silane (2, 367.09 mg, 1.70 mmol). The mixture was purged with nitrogen gas for 5 minutes. Sodium tert-butoxide (446.64 mg, 4.65 mmol) and RuPhos-Pd-G3 (200 mg, 232.38 μmol) were added. The tube was sealed, and the reaction mixture was heated at 90° C. for 5 h. After cooling, the mixture was poured over water (50) mL) and extracted with EtOAc (2×100 mL). The combined organic layer was washed with water (100 mL) and brine (50 mL), dried over anhydrous sodium sulfate and filtered. The solvent was removed under reduced pressure and the residue purified by flash silica gel column chromatography (30)-40% EtOAc in petroleum ether) to afford 5-[4-[tert-butyl(dimethyl)silyl]oxy-1-piperidyl]-1-(2,6-dibenzyloxy-3-pyridyl)-3-methyl-benzimidazol-2-one (4, 600 mg, 862.83 μmol, 56% yield) as a pale yellow solid. LCMS (ES+): m/z 651.3 [M+H]⁺.

### Step 3: 1-(2,6-dibenzyloxy-3-pyridyl)-5-(4-hydroxy-1-piperidyl)-3-methyl-benzimidazol-2-one (5)

Into a 25 mL pressure tube containing a well-stirred solution of 5-[4-[tert-butyl(dimethyl)silyl]oxy-1-piperidyl]-1-(2,6-dibenzyloxy-3-pyridyl)-3-methyl-benzimidazol-2-one (4, 600 mg, 921.83 μmol) in THF (4 mL) was added tetrabutylammonium fluoride (1M in THF) (921.83 μmol, 1 mL) at RT. The tube was sealed, and the reaction was heated to 60° C. for 18 h. The mixture was cooled to RT and poured into water (40 mL) and extracted with DCM (2×60 mL). The organic layer was dried over anhydrous sodium sulfate, filtered, and solvent removed under reduced pressure. The residue was purified by flash silica gel column chromatography (80-100% EtOAc in petroleum ether) to afford 1-(2,6-dibenzyloxy-3-pyridyl)-5-(4-hydroxy-1-piperidyl)-3-methyl-benzimidazol-2-one (5, 370) mg, 667.44 μmol, 72% yield) as an off-white solid. LCMS (ES+): m/z 537.2 [M+H]⁺.

### Step 4: tert-butyl 2-[[1-[1-(2,6-dibenzyloxy-3-pyridyl)-3-methyl-2-oxo-benzimidazol-5-yl]-4-piperidyl]oxy]acetate (7)

Into a 50 mL single neck round bottom flask containing a well-stirred solution of 1-(2,6-dibenzyloxy-3-pyridyl)-5-(4-hydroxy-1-piperidyl)-3-methyl-benzimidazol-2-one (5, 310 mg, 577.69 μmol) in anhydrous THF (10 mL) were added potassium tert-butoxide (194.47 mg, 1.73 mmol) and tert-butyl 2-bromoacetate (6, 123.95 mg, 635.46 μmol, 93.19 μL). The resulting mixture was stirred at RT for 16 h. The mixture was cooled to RT, diluted with EtOAc (40 mL) and washed with water (3×30 mL). The organic layer was dried over anhydrous sodium sulfate and the solvent was removed under reduced pressure. The residue was purified by flash silica gel column chromatography (50% EtOAc in petroleum ether r) to obtain tert-butyl 2-[[1-[1-(2,6-dibenzyloxy-3-pyridyl)-3-methyl-2-oxo-benzimidazol-5-yl]-4-piperidyl]oxy]acetate (7, 185 mg, 252.58 μmol, 44% yield) as a pale yellow solid. LCMS (ES+): m/z 651.2 [M+H]⁺.

### Step 5: tert-butyl 2-[[1-[1-(2,6-dioxo-3-piperidyl)-3-methyl-2-oxo-benzimidazol-5-yl]-4-piperidyl]oxy]acetate (8)

Into a 25 mL single neck round bottom flask containing a well-stirred solution of tert-butyl 2-[[1-[1-(2,6-dibenzyloxy-3-pyridyl)-3-methyl-2-oxo-benzimidazol-5-yl]-4-piperidyl]oxy]acetate (7, 185 mg, 284.28 μmol) in 1,4-dioxane (8 mL) was added palladium hydroxide (20% on carbon) (90 mg, 284.28 μmol) and stirred under hydrogen atmosphere for 16 h at RT. The reaction mixture was filtered through Celite, washed with 1,4-dioxane (20 mL) and DCM (10 mL) and the filtrate was concentrated under reduced pressure to obtain tert-butyl 2-[[1-[1-(2,6-dioxo-3-piperidyl)-3-methyl-2-oxo-benzimidazol-5-yl]-4-piperidyl]oxy]acetate (8, 100 mg, 150.36 μmol, 53% yield) as a pale yellow solid. LCMS (ES+): m/z 473.0 [M+H]⁺.

### Step 6: 2-[[1-[1-(2,6-dioxo-3-piperidyl)-3-methyl-2-oxo-benzimidazol-5-yl]-4-piperidyl]oxy]acetic acid (9)

Into a 10 mL single neck round bottom flask containing a well-stirred solution of tert-butyl 2-[[1-[1-(2,6-dioxo-3-piperidyl)-3-methyl-2-oxo-benzimidazol-5-yl]-4-piperidyl]oxy]acetate (8, 100 mg, 211.63 μmol) in DCM (1 mL) was added trifluoroacetic acid (740.00 mg, 6.49 mmol, 0.5 mL) at 0° C. The reaction mixture was stirred for 3 h at RT. The volatiles were removed under reduced pressure and the residue was triturated with diethyl ether (2×5 mL), filtered and dried to afford 2-[[1-[1-(2,6-dioxo-3-piperidyl)-3-methyl-2-oxo-benzimidazol-5-yl]-4-piperidyl]oxy]acetic acid (9, 70 mg, 88.51 μmol, 42% yield, TFA salt) as a pale yellow solid. LCMS (ES+): m/z 416.9 [M+H]⁺.

### Preparation of Additional Exemplary Formula (I) Compounds

### 3-[5-[1-[3-[3-[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 72)

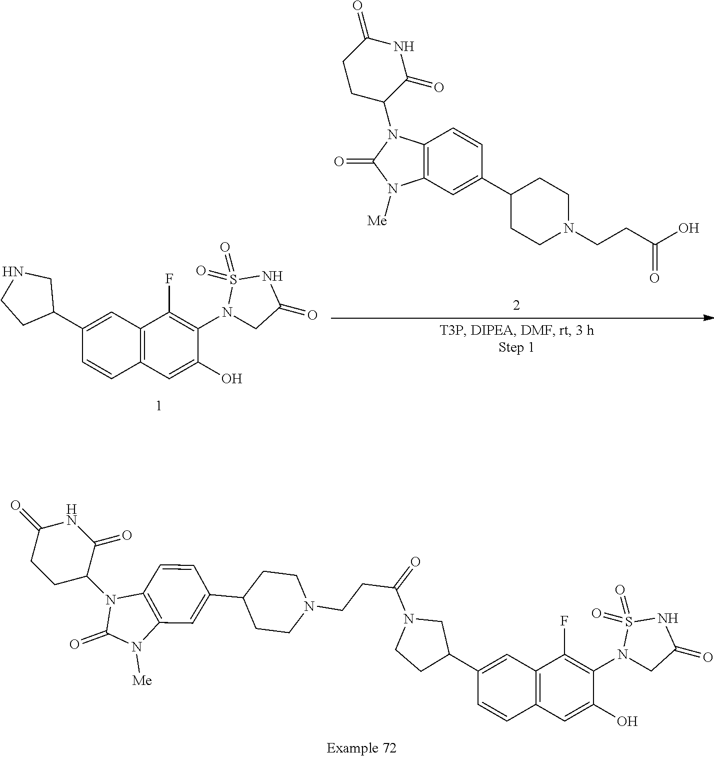 [SMILES: O=C1CN(c2c(O)cc3ccc(C4CCNC4)cc3c2F)S(=O)(=O)N1 ; Cn1c(=O)n(C2CCC(=O)NC2=O)c2ccc(C3CCN(CCC(=O)O)CC3)cc21 ; Cn1c(=O)n(C2CCC(=O)NC2=O)c2ccc(C3CCN(CCC(=O)N4CCC(c5ccc6cc(O)c(N7CC(=O)NS7(=O)=O)c(F)c6c5)C4)CC3)cc21]

### Step 1: 3-[5-[1-[3-[3-[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 72)

Into a 10 mL single neck, round bottom flask containing a well-stirred solution of 3-[4-[1-(2,6-dioxo-3-piperidyl)-3-methyl-2-oxo-benzimidazol-5-yl]-1-piperidyl]propanoic acid (2, 150 mg, 283.83 μmol, TFA salt) and 5-(1-fluoro-3-hydroxy-7-pyrrolidin-3-yl-2-naphthyl)-1,1-dioxo-1,2,5-thiadiazolidin-3-one (1, 136.07 mg, 283.83 μmol, TFA salt) in anhydrous DMF (2 mL) were added DIPEA (183.41 mg, 1.42 mmol, 247.19 μL) and 1-propanephosphonic anhydride (50 wt % in EtOAc) (198.68 mg, 312.22 μmol). The mixture was stirred at RT for 3 h. The solvent was removed under reduced pressure and the residue was purified by reverse phase preparative HPLC [Column: X-SELECT-C18 (19×150 mm) 5.0 μm; Mobile phase A: 0.1% TFA in water; Mobile phase B: MeCN) to afford 3-[5-[1-[3-[3-[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 72, 58 mg, 63.91 μmol, 23% yield, TFA salt) as an off-white solid. LCMS (ES+): m/z 762.3 [M+H]^{+ 1}H NMR (400 MHZ, DMSO-d₆) δ 11.11 (s, 1H), 10.20 (s, 1H), 9.07 (s, 1H), 7.81-7.71 (m, 2H), 7.53-7.45 (m, 1H), 7.10-7.03 (m, 3H), 6.95-6.89 (m, 1H), 5.40-5.30 (m, 1H), 4.28 (d, J=2.6 Hz, 2H), 4.06-3.88 (m, 1H), 3.77-3.63 (m, 5H), 3.42-3.29 (m, 7H), 3.17-3.05 (m, 2H), 2.96-2.80 (m, 4H), 2.76-2.56 (m, 2H), 2.44-2.29 (m, 1H), 2.20-2.09 (m, 1H), 2.06-1.88 (m, 6H).

### 3-[5-[3,3-difluoro-1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (Example 73)

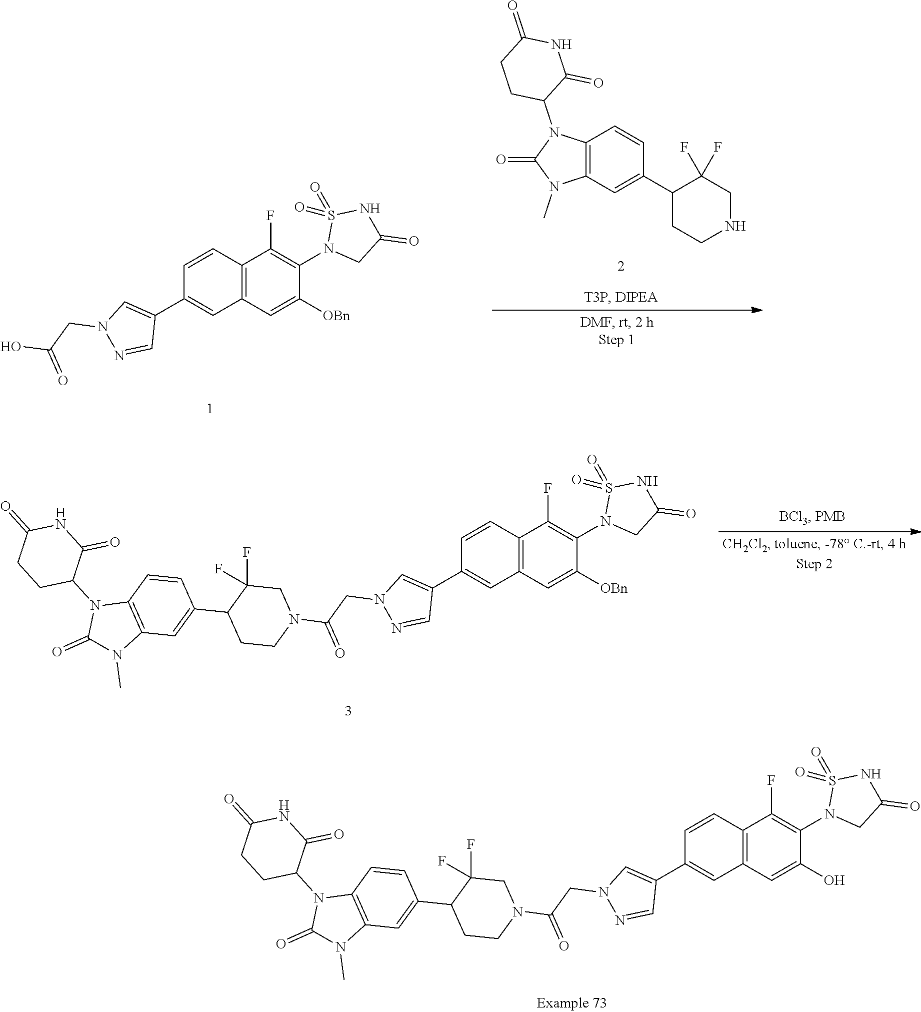 [SMILES: O=C(O)Cn1cc(-c2ccc3c(F)c(N4CC(=O)NS4(=O)=O)c(OCc4ccccc4)cc3c2)cn1 ; Cn1c(=O)n(C2CCC(=O)NC2=O)c2ccc(C3CCNCC3(F)F)cc21 ; Cn1c(=O)n(C2CCC(=O)NC2=O)c2ccc(C3CCN(C(=O)Cn4cc(-c5ccc6c(F)c(N7CC(=O)NS7(=O)=O)c(OCc7ccccc7)cc6c5)cn4)CC3(F)F)cc21 ; Cn1c(=O)n(C2CCC(=O)NC2=O)c2ccc(C3CCN(C(=O)Cn4cc(-c5ccc6c(F)c(N7CC(=O)NS7(=O)=O)c(O)cc6c5)cn4)CC3(F)F)cc21]

### Step 1: 3-[5-[1-[2-[4-[7-benzyloxy-5-fluoro-6-(1,1,4-trioxo-1,2,5-thiadiazolidin-2-yl)-2-naphthyl]pyrazol-1-yl]acetyl]-3,3-difluoro-4-piperidyl]-3-methyl-2-oxo-benzimidazol-1-yl]piperidine-2,6-dione (3)

Into a 20 mL vial containing a well-stirred solution of 2-[4-[7-benzyloxy-5-fluoro-6-(1,1,4-trioxo-1,2,5-thiadiazolidin-2-yl)-2-naphthyl]pyrazol-1-yl]acetic acid (1, 280 mg, 438.03 μmol, TFA salt) and 3-[5-(3,3-difluoro-4-piperidyl)-3-methyl-2-oxo-benzimidazol-1-yl]piperidine-2,6-dione (2, 231.92 mg, 438.03 μmol, TFA salt) in DMF (3 mL) was added DIPEA (283.06 mg, 2.19 mmol, 381.48 μL) followed by propylphosphonic anhydride solution (50 wt % in EtOAc) (417.89 mg, 656.68 μmol). After 2 h the solvent was removed under reduced pressure and the residue was purified by reverse-phase column chromatography (120 g of C18 column; 0.1% TFA in water and MeCN) to afford 3-[5-[1-[2-[4-[7-benzyloxy-5-fluoro-6-(1,1,4-trioxo-1,2,5-thiadiazolidin-2-yl)-2-naphthyl]pyrazol-1-yl]acetyl]-3,3-difluoro-4-piperidyl]-3-methyl-2-oxo-benzimidazol-1-yl]piperidine-2,6-dione (3, 200 mg, 198.40 μmol, 45% yield, TFA salt) as an off-white solid. LCMS (ES+): m/z 871.0 [M+H]⁺.

### Step 2: 3-[5-[3,3-difluoro-1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (Example 73)

Into a 25 mL round bottom flask containing a well-stirred solution of 3-[5-[1-[2-[4-[7-benzyloxy-5-fluoro-6-(1,1,4-trioxo-1,2,5-thiadiazolidin-2-yl)-2-naphthyl]pyrazol-1-yl]acetyl]-3,3-difluoro-4-piperidyl]-3-methyl-2-oxo-benzimidazol-1-yl]piperidine-2,6-dione (3, 150 mg, 149.26 μmol, TFA salt) in DCM (2.5 mL) and toluene (2.5 mL) was added pentamethylbenzene (110.63 mg, 746.29 μmol) and the reaction mixture was cooled to −78° C. BCl₃ solution (1.0 M in methylene chloride) (349.26 mg, 2.99 mmol, 3 mL) was added dropwise over a period of 2 min. Subsequently, the reaction mixture was brought to RT and stirred for 4 h. The reaction mixture was cooled to −78° C. and quenched slowly with 10% DCM in methanol (2 mL). The reaction mixture was allowed to come to RT and concentrated under reduced pressure at 30° C. The residue was purified by reverse-phase preparative HPLC [Column: X Select C18 (250×19) mm, 5 micron; 0.1% TFA in water: MeCN] to afford 3-[5-[3,3-difluoro-1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (Example 73, 31.5 mg, 33.22 μmol, 22% yield, TFA salt) as an off-white solid. LCMS (ES+): m/z 781.0 [M+H]^{+ 1}H NMR (400 MHZ, DMSO-d₆) δ 11.12 (s, 1H), 10.47 (s, 1H), 8.30 (d, J=8.1 Hz, 1H), 8.08 (d, J=6.9 Hz, 1H), 8.03-7.97 (m, 1H), 7.90 (d, J=8.6 Hz, 1H), 7.71-7.63 (m, 1H), 7.18-7.05 (m, 3H), 6.99 (d, J=8.2 Hz, 1H), 5.43-5.10 (m, 3H), 4.69 (t, J=12.3 Hz, 1H), 4.52 (d, J=12.9 Hz, 1H), 4.41 (s, 2H), 4.20-4.11 (m, 1H), 3.35 (d, J=5.9 Hz, 3H), 3.28-3.12 (m, 1H), 2.97-2.84 (m, 2H), 2.79-2.58 (m, 3H), 2.25 (d, J=13.0 Hz, 1H), 2.13-1.84 (m, 2H).

### 3-[5-[1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (Example 74)

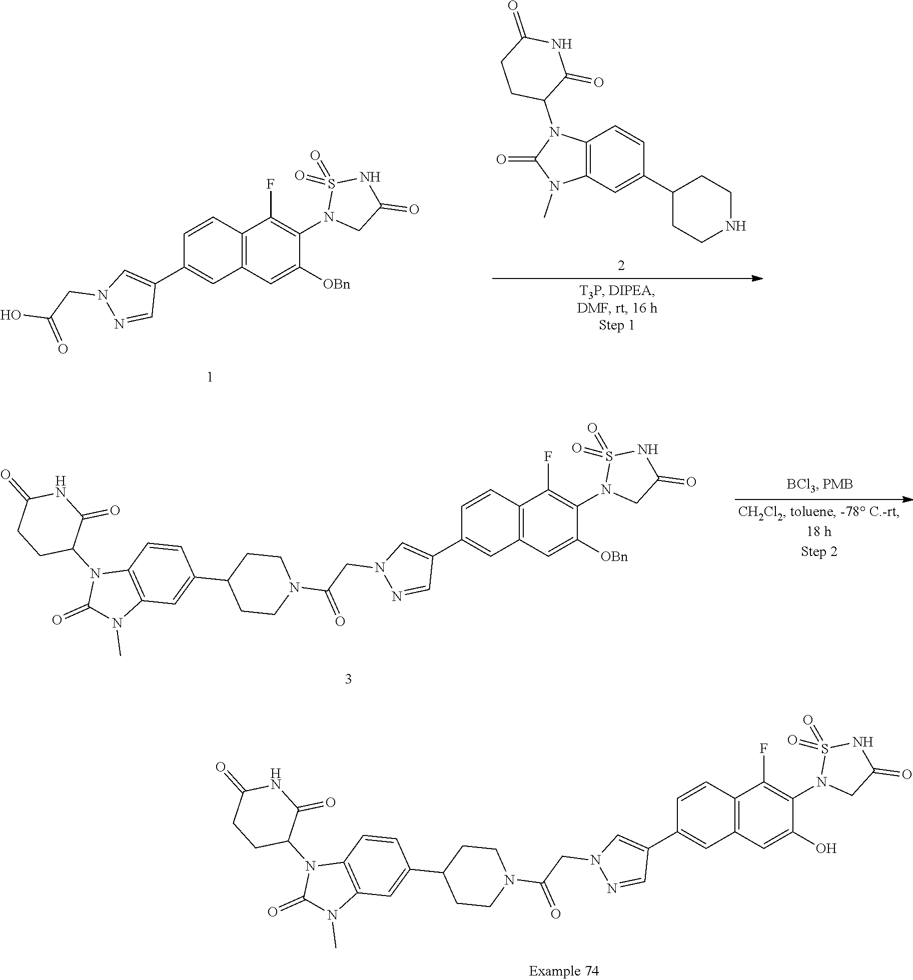 [SMILES: O=C(O)Cn1cc(-c2ccc3c(F)c(N4CC(=O)NS4(=O)=O)c(OCc4ccccc4)cc3c2)cn1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)Cn4cc(-c5ccc6c(F)c(N7CC(=O)NS7(=O)=O)c(OCc7ccccc7)cc6c5)cn4)CC3)cc21 ; Cn1c(=O)n(C2CCC(=O)NC2=O)c2ccc(C3CCN(C(=O)Cn4cc(-c5ccc6c(F)c(N7CC(=O)NS7(=O)=O)c(O)cc6c5)cn4)CC3)cc21]

### Step 1: 3-[5-[1-[2-[4-[7-benzyloxy-5-fluoro-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (3)

To a solution of 2-[4-[7-benzyloxy-5-fluoro-6-(1,1,4-trioxo-1,2,5-thiadiazolidin-2-yl)-2-naphthyl]pyrazol-1-yl]acetic acid (1, 130 mg, 208.16 μmol, TFA salt) and 3-[3-methyl-2-oxo-5-(4-piperidyl)benzimidazol-1-yl]piperidine-2,6-dione (2, 118.29 mg, 312.24 μmol, HCl salt) in DMF (5 mL) was added DIPEA (26.90 mg, 208.16 μmol, 36.26 μL), followed by propanephosphonic anhydride (132.47 mg, 416.32 μmol) and the reaction mixture was stirred for 2 h. The reaction mixture was concentrated under reduced pressure and the residue purified by reverse phase column chromatography (0.1% TFA in water:MeCN) to afford 3-[5-[1-[2-[4-[7-benzyloxy-5-fluoro-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (3, 140 mg, 114.35 μmol, 55% yield, TFA salt) as a white powder. LCMS (ES−): m/z 834.0 [M−H]⁻.

### Step 2: 3-[5-[1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (Example 74)

Into a 10 mL single neck round bottom flask containing a well-stirred solution of 3-[5-[1-[2-[4-[7-benzyloxy-5-fluoro-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (3, 350 mg, 368.85 μmol, TFA salt) in a 1:1 mixture of anhydrous DCM (6 mL) and anhydrous toluene (6 mL) was added pentamethylbenzene (328.08 mg, 2.21 mmol) and the reaction mixture was cooled to −78° C. BCl₃ (1.0 M Solution in DCM) (5.0 mmol, 5 mL) was added dropwise and the resulting mixture was stirred at RT for 18 h. The reaction was cooled to −78° C. and quenched with 10% MeOH in DCM (7 mL). The reaction mixture was concentrated under reduced pressure and the residue purified by reverse phase preparative HPLC (Column: Silicycle-C18-120 g; Mobile Phase A: 0.1% TFA in water and Mobile Phase B: MeCN) to afford 3-[5-[1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-3-methyl-2-oxo-benzimidazol-1-yl]piperidine-2,6-dione (Example 74, 37.2 mg, 39.87 μmol, 11% yield, TFA salt) as a white solid. LCMS (ES+): m/z 745.3 [M+H]^{+ 1}H NMR (400 MHZ, DMSO-d₆) δ 11.09 (s, 1H), 10.43 (s, 1H), 8.27 (s, 1H), 8.06 (s, 1H), 7.99 (s, 1H), 7.89 (d, J=8.7 Hz, 1H), 7.72-7.61 (m, 1H), 7.14-7.06 (m, 2H), 7.03 (d, J=8.1 Hz, 1H), 6.97-6.90 (m, 1H), 5.34 (dd, J=12.7, 5.4 Hz, 1H), 5.30-5.16 (m, 2H), 4.52 (d, J=13.1 Hz, 1H), 4.39 (s, 2H), 4.08 (d, J=13.4 Hz, 1H), 3.33 (s, 3H), 3.24-3.14 (m, 1H), 2.97-2.80 (m, 2H), 2.77-2.58 (m, 3H), 2.04-1.94 (m, 1H), 1.91-1.78 (m, 2H), 1.77-1.64 (m, 1H), 1.63-1.50 (m, 1H).

### N-[[4-[[1-(2,6-dioxo-3-piperidyl)-3-methyl-2-oxo-benzimidazol-5-yl]cyclohexyl]methyl]-5-fluoro-7-hydroxy-6-(1,1,4-trioxo-1,2,5-thiadiazolidin-2-yl)naphthalene-2-carboxamide (Example 75) Relative Stereochemistry Around Cyclohexane Ring Arbitrarily Assigned

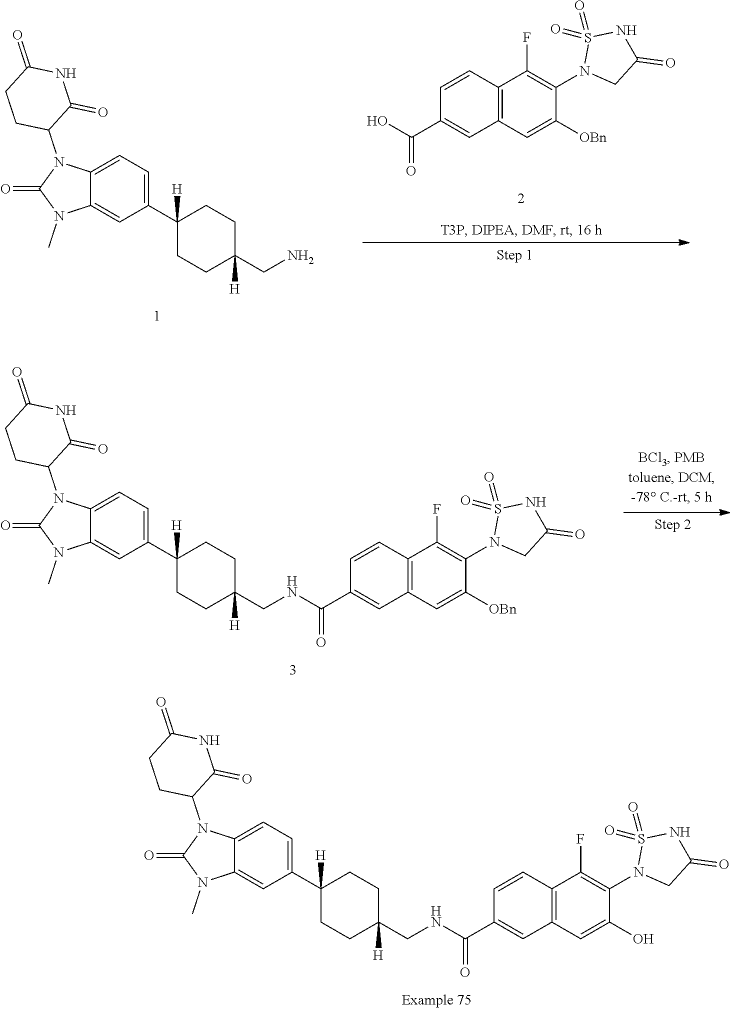 [SMILES: Cn1c(=O)n(C2CCC(=O)NC2=O)c2ccc([C@H]3CC[C@@H](CN)CC3)cc21 ; O=C1CN(c2c(OCc3ccccc3)cc3cc(C(=O)O)ccc3c2F)S(=O)(=O)N1 ; Cn1c(=O)n(C2CCC(=O)NC2=O)c2ccc([C@H]3CC[C@@H](CNC(=O)c4ccc5c(F)c(N6CC(=O)NS6(=O)=O)c(OCc6ccccc6)cc5c4)CC3)cc21 ; Cn1c(=O)n(C2CCC(=O)NC2=O)c2ccc([C@H]3CC[C@@H](CNC(=O)c4ccc5c(F)c(N6CC(=O)NS6(=O)=O)c(O)cc5c4)CC3)cc21]

### Step 1: 7-benzyloxy-N-[[4-[[1-(2,6-dioxo-3-piperidyl)-3-methyl-2-oxo-benzimidazol-5-yl]cyclohexyl]methyl]-5-fluoro-6-(1,1,4-trioxo-1,2,5-thiadiazolidin-2-yl)naphthalene-2-carboxamide (3)

Into a 50 mL single neck round bottom flask containing a well-stirred solution of 7-benzyloxy-5-fluoro-6-(1,1,4-trioxo-1,2,5-thiadiazolidin-2-yl)naphthalene-2-carboxylic acid (2, 126.10 mg, 272.46 μmol) in DMF (7 mL) were added 3-[5-[4-(aminomethyl)cyclohexyl]-3-methyl-2-oxo-benzimidazol-1-yl]piperidine-2,6-dione (1, 121.21 mg, 247.69 μmol), N,N-diisopropylethylamine (222.60 mg, 1.72 mmol, 0.3 mL) and propylphosphonic anhydride solution (50% in EtOAc) (477.63 mg, 1.50 mmol, 0.3 mL). After 16 h, the reaction mixture was concentrated under reduced pressure. The residue was purified by reverse phase HPLC [Purification method: Siliasep premium C18, 25 μm, 120 g; Mobile phase A: 0.1% TFA in Water, Mobile phase B: MeCN] to afford 7-benzyloxy-N-[[4-[[1-(2,6-dioxo-3-piperidyl)-3-methyl-2-oxo-benzimidazol-5-yl]cyclohexyl]methyl]-5-fluoro-6-(1,1,4-trioxo-1,2,5-thiadiazolidin-2-yl)naphthalene-2-carboxamide (3, 110 mg, 138.18 μmol, 56% yield) as a pale yellow solid. LCMS (ES+): m/z 783.2 [M+H]⁺.

### Step 2: N-[[4-[[1-(2,6-dioxo-3-piperidyl)-3-methyl-2-oxo-benzimidazol-5-yl]cyclohexyl]methyl]-5-fluoro-7-hydroxy-6-(1,1,4-trioxo-1,2,5-thiadiazolidin-2-yl)naphthalene-2-carboxamide (Example 75)

Into a 50 mL single neck round bottom flask containing a well-stirred solution of 7-benzyloxy-N-[[4-[1-(2,6-dioxo-3-piperidyl)-3-methyl-2-oxo-benzimidazol-5-yl]cyclohexyl]methyl]-5-fluoro-6-(1,1,4-trioxo-1,2,5-thiadiazolidin-2-yl)naphthalene-2-carboxamide (3, 91.84 mg, 114.97 μmol) in DCM (3 mL) and toluene (3 mL) was added pentamethylbenzene (85.22 mg, 574.83 μmol, 92.93 μL) at RT. BCl₃ solution (1.0 M in DCM) (2.34 mmol, 2.34 mL) was added at −75° C. and then stirred at RT. After 5 h the reaction was quenched with 5% MeOH in DCM (2.0 mL) at −75° C., concentrated under reduced pressure, the residue was triturated with diethyl ether (10 mL), filtered and purified by reverse phase prep HPLC [Purification method: Column: X-Select C18 (19×150 mm), 5 micron; Mobile phase A: 0.1% TFA in water; Mobile phase B: MeCN] to afford N-[[4-[[1-(2,6-dioxo-3-piperidyl)-3-methyl-2-oxo-benzimidazol-5-yl]cyclohexyl]methyl]-5-fluoro-7-hydroxy-6-(1,1,4-trioxo-1,2,5-thiadiazolidin-2-yl)naphthalene-2-carboxamide (Example 75, 31 mg, 41.87 μmol, 36.41% yield, 93.55% purity) as an off white solid. LCMS (ES+): m/z 693.1 [M+H]^{+ 1}H NMR (300 MHz, DMSO-d₆) δ 11.08 (s, 1H), 10.72 (s, 1H), 8.75-8.64 (m, 1H), 8.28 (s, 1H), 7.98 (d, J=8.7 Hz, 1H), 7.80 (d, J=8.7 Hz, 1H), 7.21 (s, 1H), 7.07 (s, 1H), 6.99 (d, J=8.1 Hz, 1H), 6.90 (d, J=8.1 Hz, 1H), 5.38-5.25 (m, 1H), 4.44 (s, 2H), 3.32 (s, 3H), 3.26-3.16 (m, 3H), 3.00-2.80 (m, 1H), 2.76-2.56 (m, 2H), 2.08-1.78 (m, 5H), 1.75-1.62 (m, 1H), 1.59-1.40 (m, 2H), 1.27-1.03 (m, 2H).

### N-[[4-[[1-(2,6-dioxo-3-piperidyl)-3-methyl-2-oxo-benzimidazol-5-yl]cyclohexyl]methyl]-5-fluoro-7-hydroxy-6-(1,1,4-trioxo-1,2,5-thiadiazolidin-2-yl)naphthalene-2-carboxamide (Example 76)—relative Stereochemistry Around Cyclohexane Ring Arbitrarily Assigned

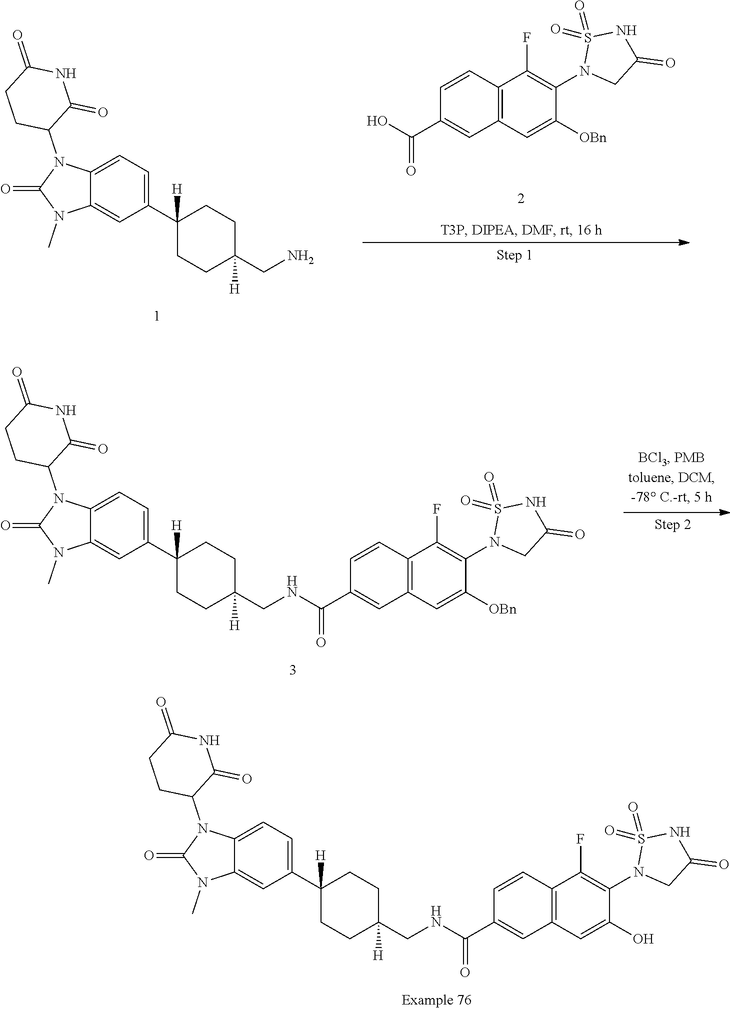 [SMILES: Cn1c(=O)n(C2CCC(=O)NC2=O)c2ccc([C@H]3CC[C@H](CN)CC3)cc21 ; O=C1CN(c2c(OCc3ccccc3)cc3cc(C(=O)O)ccc3c2F)S(=O)(=O)N1 ; Cn1c(=O)n(C2CCC(=O)NC2=O)c2ccc([C@H]3CC[C@H](CNC(=O)c4ccc5c(F)c(N6CC(=O)NS6(=O)=O)c(OCc6ccccc6)cc5c4)CC3)cc21 ; Cn1c(=O)n(C2CCC(=O)NC2=O)c2ccc([C@H]3CC[C@H](CNC(=O)c4ccc5c(F)c(N6CC(=O)NS6(=O)=O)c(O)cc5c4)CC3)cc21]

The title compound was prepared from (1) and (2) over two steps (Example 76, 48.0 mg, 63.76 μmol, 45% yield using the same procedure as Example 75). LCMS (ES+): m/z 693.2 [M+H]^{+ 1}H NMR (400 MHZ, DMSO-d₆) δ 11.09 (s, 1H), 10.76 (s, 1H), 8.69 (t, J=5.8 Hz, 1H), 8.31-8.27 (m, 1H), 7.98 (d, J=8.7 Hz, 1H), 7.81 (dd, J=8.8, 1.6 Hz, 1H), 7.21 (s, 1H), 7.12-7.09 (m, 1H), 7.03 (d, J=8.1 Hz, 1H), 6.97-6.91 (m, 1H), 5.34 (dd, J=12.7, 5.4 Hz, 1H), 4.48 (s, 2H), 3.47 (t, J=6.8 Hz, 2H), 3.35 (s, 3H), 2.98-2.83 (m, 1H), 2.78-2.57 (m, 4H), 2.10-1.95 (m, 2H), 1.86-1.69 (m, 4H), 1.68-1.55 (m, 4H).

### 2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Example 77)

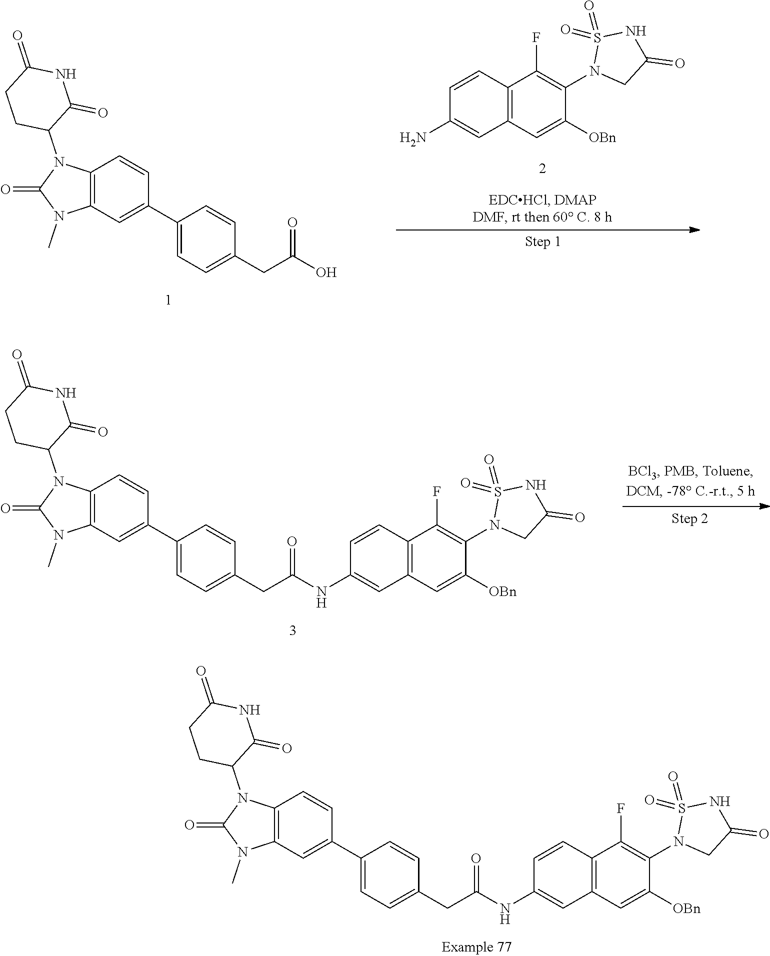 [SMILES: Cn1c(=O)n(C2CCC(=O)NC2=O)c2ccc(-c3ccc(CC(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3ccc(CC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(-c3ccc(CC(=O)Nc4ccc5c(F)c(N6CC(=O)NS6(=O)=O)c(OCc6ccccc6)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]phenyl]acetamide (3)

To a 25 mL single neck round bottom flask containing a well-stirred solution of 2-[4-[1-(2,6-dioxo-3-piperidyl)-3-methyl-2-oxo-benzimidazol-5-yl]phenyl]acetic acid (1, 230 mg, 555.43 μmol) in DMF (2 mL) were added 4-dimethylaminopyridine (339.28 mg, 2.78 mmol), N-(3-dimethylaminopropyl)-N′-ethylcarbodiimide hydrochloride (159.71 mg, 833.14 μmol). After 2 h, a solution of 5-(6-amino-3-benzyloxy-1-fluoro-2-naphthyl)-1,1-dioxo-1,2,5-thiadiazolidin-3-one (2, 301.67 mg, 555.43 μmol, TFA salt) in DMF (1.5 mL) was added. After 16 h, additional quantities of 4-dimethylaminopyridine (339.28 mg, 2.78 mmol) and N-(3-dimethylaminopropyl)-N′-ethylcarbodiimide hydrochloride (159.71 mg, 833.14 μmol) were added. The mixture was stirred at 60° C. for 8 h. The reaction mixture was quenched with ice-water and the precipitate was filtered. The material was purified by reverse phase column chromatography [Purification method: Siliasep C18 120 g, column; Mobile phase A: 0.1% TFA in water; Mobile phase B: MeCN] to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]phenyl]acetamide (3, 170 mg, 194.78 μmol, 35% yield) as a yellow solid. LCMS (ES+): m/z 777.2 [M+H]⁺.

### Step 2: 2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Example 77)

To a 25 mL single neck round bottomed flask containing a well-stirred suspension of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]phenyl]acetamide (3, 170 mg, 194.78 μmol) in a mixture of toluene (3 mL) and DCM (3 mL), was added pentamethylbenzene (144.37 mg, 973.88 μmol, 157.44 μL) under nitrogen. The resulting suspension was cooled to −78° C. and BC₃; solution (1.0M in methylene chloride) (456.44 mg, 3.90 mmol, 3.9 mL) was added dropwise and the reaction was stirred at RT. The mixture was cooled to −78° C. and quenched with 5% MeOH in DCM (4 mL) and concentrated under reduced pressure. The residue was triturated with diethyl ether (2×25 mL) and filtered. The material was purified by reverse phase prep HPLC [Purification method: Column: X-Bridge, C18 (19×150 mm), 5 Micron; Mobile phase A: 0.1% TFA in water; Mobile phase B: MeCN] to afford 2-[4-[1-(2,6-dioxo-3-piperidyl)-3-methyl-2-oxo-benzimidazol-5-yl]phenyl]-N-[5-fluoro-7-hydroxy-6-(1,1,4-trioxo-1,2,5-thiadiazolidin-2-yl)-2-naphthyl]acetamide (Example 77, 52 mg, 73.87 μmol, 38% yield) as a pale brown solid. LCMS (ES−): m/z 684.8 [M−H]^{- 1}H NMR (400 MHZ, DMSO-d₆) δ 11.12 (s, 1H), 10.45 (s, 1H), 8.18 (d, J=1.9 Hz, 1H), 7.87 (d, J=9.0 Hz, 1H), 7.70-7.62 (m, 2H), 7.53-7.41 (m, 4H), 7.35 (dd, J=8.3, 1.7 Hz, 1H), 7.19 (d, J=8.5 Hz, 1H), 6.97-6.94 (m, 1H), 5.40 (dd, J=12.9, 5.4 Hz, 1H), 4.40 (s, 2H), 3.74 (s, 2H), 3.41 (s, 3H), 2.98-2.85 (m, 1H), 2.81-2.70 (m, 1H), 2.69-2.59 (m, 1H), 2.10-1.99 (m, 1H).

### N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)acetamide (Example 78)

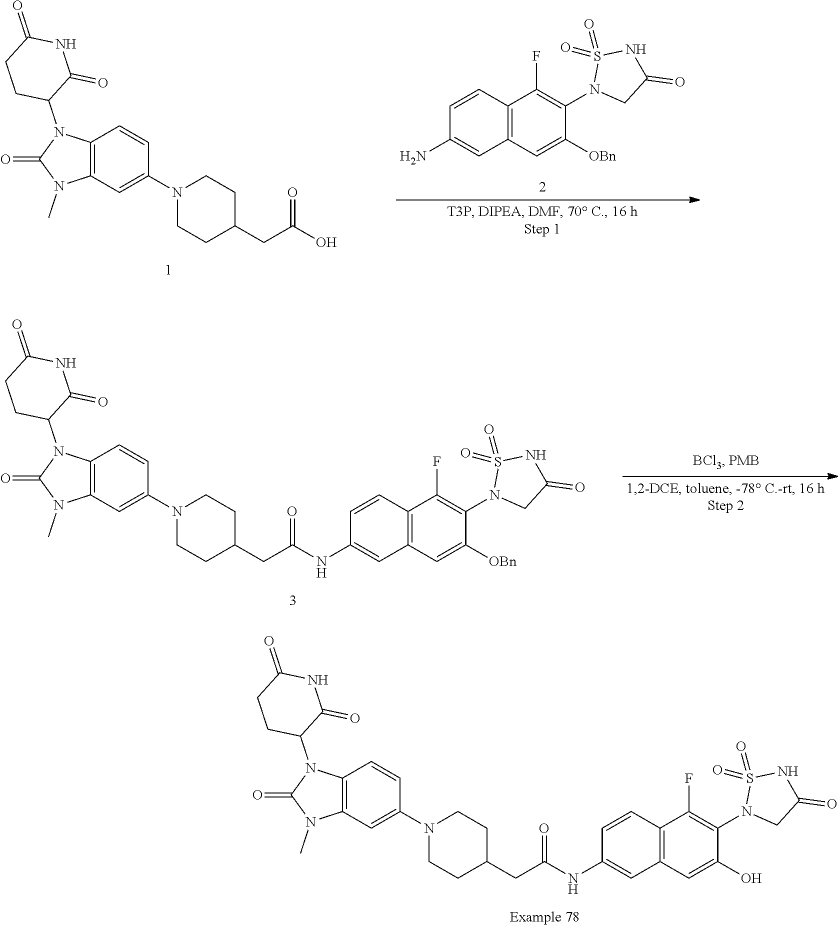 [SMILES: Cn1c(=O)n(C2CCC(=O)NC2=O)c2ccc(N3CCC(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-4-piperidyl]acetamide (3)

Into a 50 mL single neck, round bottom flask containing a well-stirred solution of 2-(1-(1-(2,6-dioxopiperidin-3-yl)-3-methyl-2-oxo-2,3-dihydro-1H-benzo[d]imidazol-5-yl)piperidin-4-yl)acetic acid (1, 300 mg, 601.61 μmol) in dry DMF (6 mL) was added propylphosphonic anhydride solution (50 wt % in EtOAc) (765.68 mg, 1.20 mmol) followed by DIPEA (388.76 mg, 3.01 mmol, 523.93 μL). The reaction mixture was stirred at RT for 1 h, then 5-(6-amino-3-benzyloxy-1-fluoro-2-naphthyl)-1,1-dioxo-1,2,5-thiadiazolidin-3-one (2, 316.74 mg, 601.61 μmol, TFA salt) was added and the reaction mixture was stirred for 15 h at 70° C. The reaction mixture was concentrated and the residue was purified by reverse-phase preparative HPLC [Column: X-Select C18 (150×19), 5 μm; Mobile Phase A: 0.1% TFA in water and Mobile Phase B: MeCN] to afford N-[7-benzyloxy-5-fluoro-6-(1,1,4-trioxo-1,2,5-thiadiazolidin-2-yl)-2-naphthyl]-2-[[1-[1-(2,6-dioxo-3-piperidyl)-3-methyl-2-oxo-benzimidazol-5-yl]-4-piperidyl]acetamide (3, 120 mg, 20% yield, TFA salt) as a brown solid. LCMS (ES+): m/z 784.3 [M+H]⁺.

### Step 2: N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)acetamide (Example 78)

Into a 25 mL single neck round bottom flask containing a well-stirred solution of N-[7-benzyloxy-5-fluoro-6-(1,1,4-trioxo-1,2,5-thiadiazolidin-2-yl)-2-naphthyl]-2-[[1-[1-(2,6-dioxo-3-piperidyl)-3-methyl-2-oxo-benzimidazol-5-yl]-4-piperidyl]acetamide (3, 110 mg, 111.73 μmol, TFA salt) in anhydrous DCE (4 mL) and toluene (4 mL) was added pentamethylbenzene (82.82 mg, 558.67 μmol). The reaction mixture was cooled to −78° C. and BCl₃ (1.0 M solution in CH₂Cl₂) (327.30 mg, 2.79 mmol, 2.79 mL) was added. The reaction mixture was stirred at RT for 16 h. The reaction mixture was quenched with 5% MeOH in DCM (5 mL) at −78° C. The volatiles were removed and residue triturated with diethyl ether (10 mL) and purified by reverse-phase preparative HPLC [Column: X-Select C18 (150×19), 5 μm; Mobile Phase A: 0.1% TFA in water and Mobile Phase B: MeCN] to afford N-(6-(1,1-dioxido-4-oxo-1,2,5-thiadiazolidin-2-yl)-5-fluoro-7-hydroxynaphthalen-2-yl)-2-(1-(1-(2,6-dioxopiperidin-3-yl)-3-methyl-2-oxo-2,3-dihydro-1H-benzo[d]imidazol-5-yl)piperidin-4-yl)acetamide (Example 78, 41 mg, 45% yield, TFA salt) as an off-white solid. LCMS (ES+): m/z 694.0 [M+H]^{+ 1}H NMR (400 MHZ, DMSO-d₆) δ 11.12 (s, 1H), 10.21 (s, 1H), 10.05 (s, 1H), 8.19-8.15 (m, 1H), 7.85 (d, J=8.9 Hz, 1H), 7.44 (dd, J=9.0, 2.0 Hz, 1H), 7.22-7.08 (m, 2H), 6.95 (s, 1H), 5.38 (dd, J=12.9, 5.4 Hz, 1H), 4.23 (s, 2H), 3.62 (d, J=11.3 Hz, 2H), 3.36 (s, 3H), 2.90 (ddd, J=16.9, 12.7, 5.2 Hz, 1H), 2.78-2.58 (m, 2H), 2.43 (d, J=6.8 Hz, 2H), 2.23-2.08 (m, 1H), 2.05-1.88 (m, 3H), 1.71-1.53 (m, 2H).

### 3-[6-[1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-1-methyl-indazol-3-yl]piperidine-2,6-dione (Example 79)

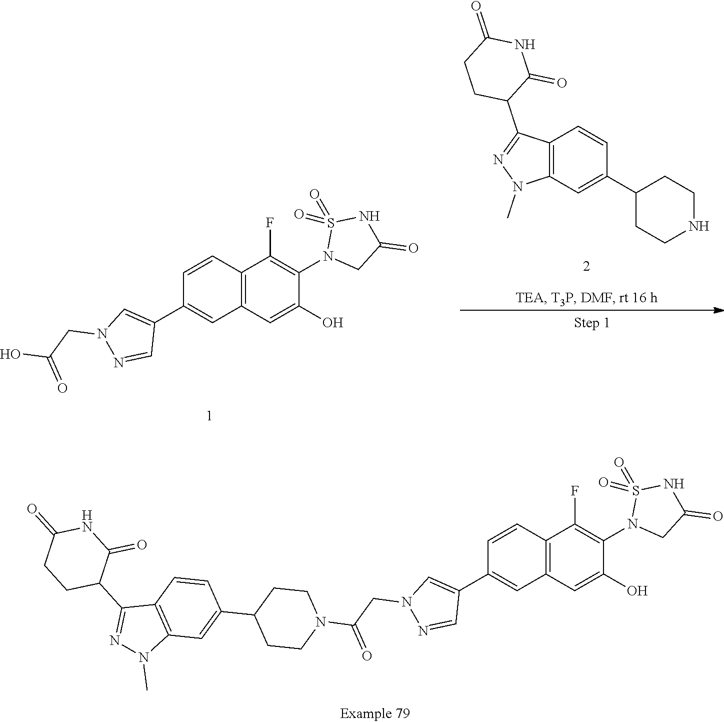 [SMILES: O=C(O)Cn1cc(-c2ccc3c(F)c(N4CC(=O)NS4(=O)=O)c(O)cc3c2)cn1 ; Cn1nc(C2CCC(=O)NC2=O)c2ccc(C3CCNCC3)cc21 ; Cn1nc(C2CCC(=O)NC2=O)c2ccc(C3CCN(C(=O)Cn4cc(-c5ccc6c(F)c(N7CC(=O)NS7(=O)=O)c(O)cc6c5)cn4)CC3)cc21]

### Step 1: 3-[6-[1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-1-methyl-indazol-3-yl]piperidine-2,6-dione (Example 79)

Into a 25 mL round bottom flask containing a well-stirred solution of 2-[4-[5-fluoro-7-hydroxy-6-(1,1,4-trioxo-1,2,5-thiadiazolidin-2-yl)-2-naphthyl]pyrazol-1-yl]acetic acid (1, 70 mg, 151.97 μmol) in DMF (3 mL) was added triethyl amine (46.13 mg, 455.90 μmol, 63.54 μL) and a solution of propylphosphonic anhydride solution (50% in EtOAc) (116.05 mg, 182.36 μmol). After 30 min, (3-(1-methyl-6-(piperidin-4-yl)-1H-indazol-3-yl)piperidine-2,6-dione (2, 68.87 mg, 151.97 μmol, TFA salt) was added. After 16 h, the volatiles were removed under reduced pressure and the residue was purified by reverse phase column chromatography [Purification method: Column: X-Select C18 (150×19), 5 micron; Mobile phase A: 0.1% TFA in water; Mobile phase B: MeCN] to afford 3-[6-[1-[2-[4-[5-fluoro-7-hydroxy-6-(1,1,4-trioxo-1,2,5-thiadiazolidin-2-yl)-2-naphthyl]pyrazol-1-yl]acetyl]-4-piperidyl]-1-methyl-indazol-3-yl]piperidine-2,6-dione (Example 79, 11 mg, 12.62 μmol, 8% yield, TFA salt) as a brown solid. LCMS (ES+): m/z 729.0 [M+H]^{+ 1}H NMR (400 MHZ, DMSO-d₆) δ 10.89 (s, 1H), 10.44 (s, 1H), 8.28 (s, 1H), 8.07 (s, 1H), 8.00 (s, 1H), 7.90 (d, J=8.6 Hz, 1H), 7.69-7.60 (m, 2H), 7.46 (s, 1H), 7.10-7.04 (m, 2H), 5.24 (d, J=4.7 Hz, 2H), 4.58-4.51 (m, 1H), 4.40 (s, 2H), 4.34 (dd, J=9.8, 5.1 Hz, 1H), 4.11 (d, J=13.4 Hz, 1H), 3.97 (s, 3H), 3.24 (t, J=12.7 Hz, 1H), 2.98 (t, J=11.9 Hz, 1H), 2.82-2.70 (m, 1H), 2.65-2.55 (m, 3H), 2.41-2.35 (m, 1H), 2.24-2.11 (m, 1H), 1.95-1.83 (m, 2H), 1.84-1.71 (m, 1H), 1.70-1.58 (m, 1H).

### 3-[5-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-3-methyl-2-oxo-benzimidazol-1-yl]piperidine-2,6-dione (Example 80)

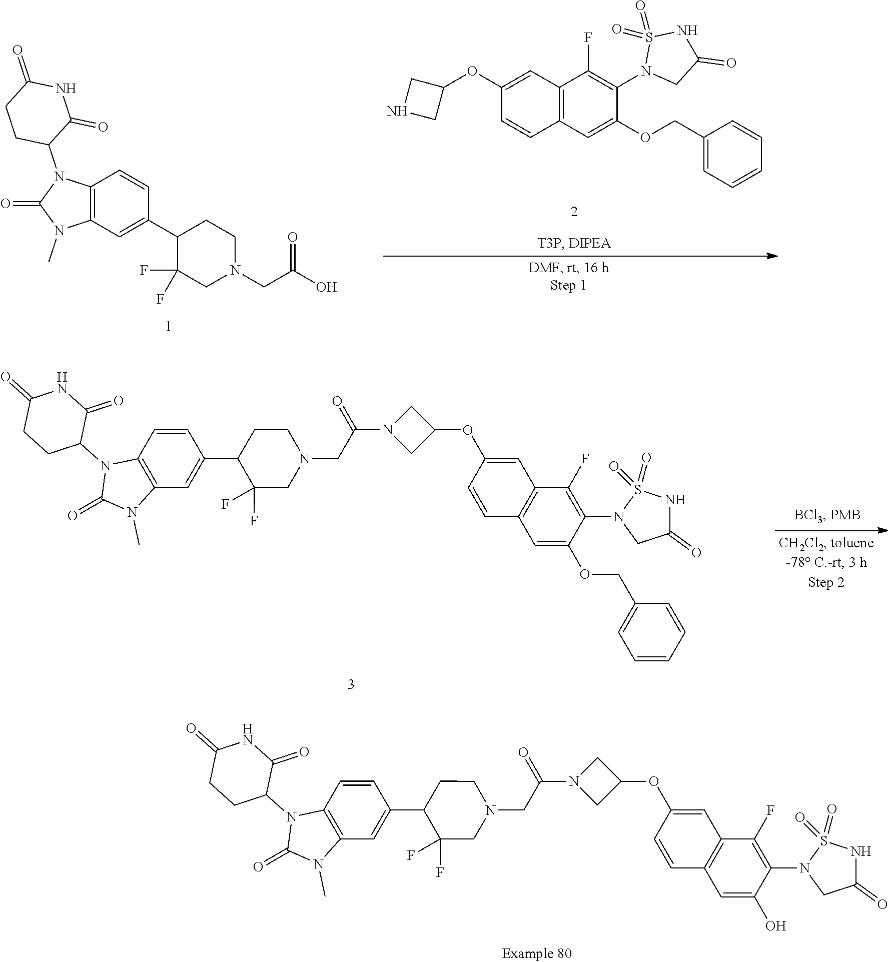 [SMILES: Cn1c(=O)n(C2CCC(=O)NC2=O)c2ccc(C3CCN(CC(=O)O)CC3(F)F)cc21 ; O=C1CN(c2c(OCc3ccccc3)cc3ccc(OC4CNC4)cc3c2F)S(=O)(=O)N1 ; Cn1c(=O)n(C2CCC(=O)NC2=O)c2ccc(C3CCN(CC(=O)N4CC(Oc5ccc6cc(OCc7ccccc7)c(N7CC(=O)NS7(=O)=O)c(F)c6c5)C4)CC3(F)F)cc21 ; Cn1c(=O)n(C2CCC(=O)NC2=O)c2ccc(C3CCN(CC(=O)N4CC(Oc5ccc6cc(O)c(N7CC(=O)NS7(=O)=O)c(F)c6c5)C4)CC3(F)F)cc21]

### Step 1: 3-[5-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-3-methyl-2-oxo-benzimidazol-1-yl]piperidine-2,6-dione (3)

Into a 20 mL vial containing a well-stirred solution of 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetic acid (1, 250 mg, 445.33 μmol, TFA salt) in DMF (3 mL) was added DIPEA (287.77 mg, 2.23 mmol, 387.84 μL) followed by and propylphosphonic anhydride solution (50 wt % in EtOAc) (212.54 mg, 668.00 μmol). After 10 min, 5-(7-(azetidin-3-yloxy)-3-(benzyloxy)-1-fluoronaphthalen-2-yl)-1,2,5-thiadiazolidin-3-one 1,1-dioxide hydrochloride (2, 219.97 mg, 445.33 μmol, HCl salt) was added and the reaction mixture was stirred for an additional 16 h. The solvent was removed under reduced pressure and ice-cold water (8 mL) was added. The precipitate was filtered, washed with toluene and dried under vacuum to afford 3-[5-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-3-methyl-2-oxo-benzimidazol-1-yl]piperidine-2,6-dione (3, 350 mg, 234.05 μmol, 53% yield) as an off-white solid. LCMS (ES+): m/z 876.3 [M+H]⁺.

### Step 2: 3-[5-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-3-methyl-2-oxo-benzimidazol-1-yl]piperidine-2,6-dione (Example 80)

Into a 25 mL round bottom flask containing a well-stirred solution of 3-[5-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-3-methyl-2-oxo-benzimidazol-1-yl]piperidine-2,6-dione (3, 350 mg, 275.73 μmol) in DCM (5 mL) and toluene (5 mL) was added pentamethylbenzene (204.38 mg, 1.38 mmol, 222.87 μL) and the reaction mixture was cooled to −78° C. BCl₃ solution (1M in DCM) (5.51 mmol, 5.51 mL) was added dropwise over a period of 2 minutes. The reaction mixture was brought to RT and stirred for 3 h, cooled to −78° C. and quenched slowly with 5% methanol in DCM (2.5 mL). The reaction mixture was allowed to attain RT and concentrated under reduced pressure at 30° C. The residue was purified by reverse-phase preparative-HPLC [Column: LUNA C18 (250×21.2) mm, 10 micron; 10 mM Ammonium acetate in water: MeCN] to afford 3-[5-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-3-methyl-2-oxo-benzimidazol-1-yl]piperidine-2,6-dione (Example 80, 95 mg, 114.90 μmol, 42% yield) as an off-white solid. LCMS (ES+): m/z 786.0 [M+H]^{+ 1}H NMR (400 MHZ, DMSO-d₆) δ 11.11 (s, 1H), 10.17 (s, 1H), 7.77 (d, J=9.1 Hz, 1H), 7.24-7.18 (m, 1H), 7.09 (d, J=6.5 Hz, 3H), 7.03-6.95 (m, 2H), 5.37 (dd, J=12.7, 5.4 Hz, 1H), 5.30-5.21 (m, 1H), 4.79-4.69 (m, 1H), 4.53-4.43 (m, 1H), 4.36 (s, 2H), 4.28-4.21 (m, 1H), 4.01-3.93 (m, 1H), 3.83-3.73 (m, 3H), 3.37-3.27 (m, 5H), 2.98-2.84 (m, 2H), 2.77-2.58 (m, 2H), 2.41-2.27 (m, 1H), 2.07-1.94 (m, 2H).

### N-(6-(1,1-dioxido-4-oxo-1,2,5-thiadiazolidin-2-yl)-5-fluoro-7-hydroxynaphthalen-2-yl)-2-((1s,4s)-4-(1-(2,6-dioxopiperidin-3-yl)-3-methyl-2-oxo-2,3-dihydro-1H-benzo[d]imidazol-5-yl)cyclohexyl)acetamide (Example 81a) and N-(6-(1,1-dioxido-4-oxo-1,2,5-thiadiazolidin-2-yl)-5-fluoro-7-hydroxynaphthalen-2-yl)-2-((1r,4r)-4-(1-(2,6-dioxopiperidin-3-yl)-3-methyl-2-oxo-2,3-dihydro-1H-benzo[d]imidazol-5-yl)cyclohexyl)acetamide (Example 81b,) Relative Stereochemistry Around Cyclohexane Ring Arbitrarily Assigned

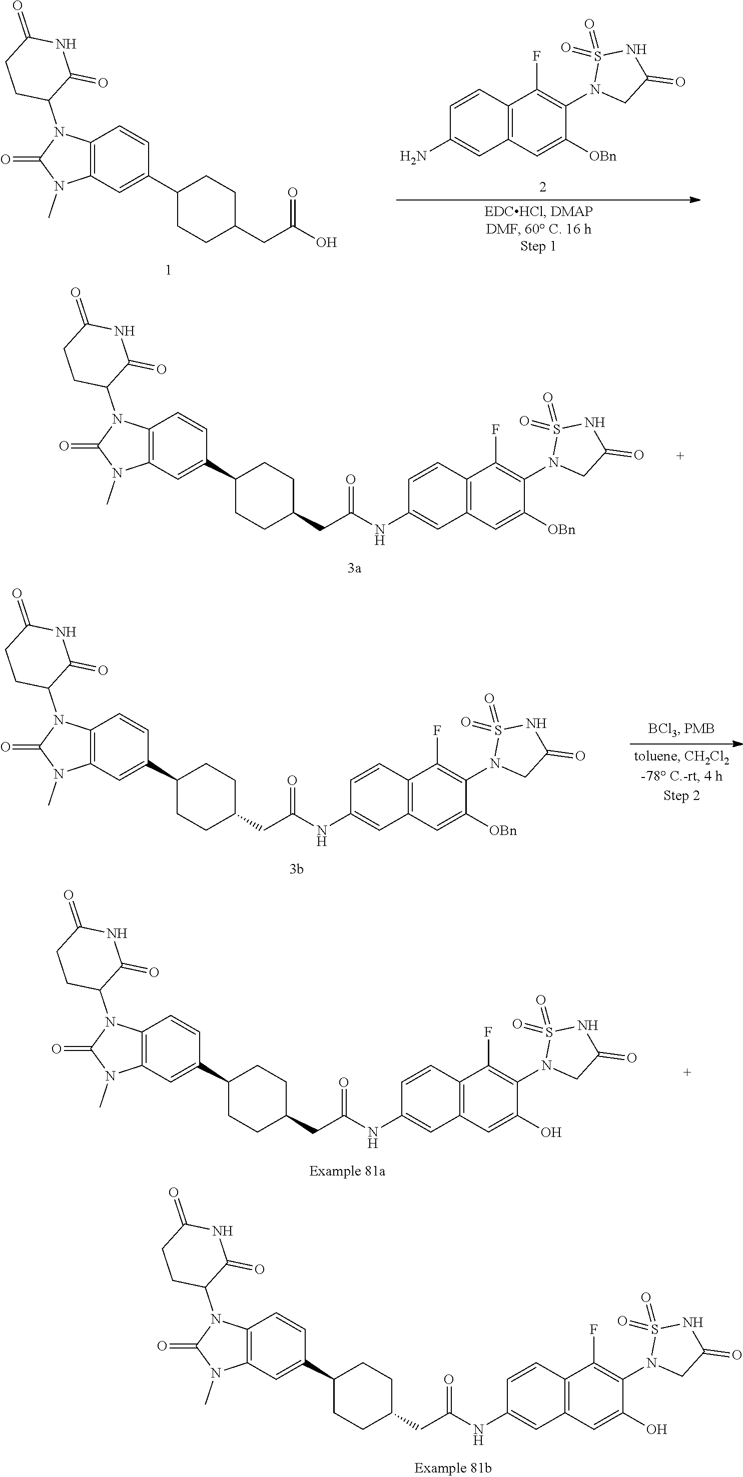

### Step 1: N-(7-(benzyloxy)-6-(1,1-dioxido-4-oxo-1,2,5-thiadiazolidin-2-yl)-5-fluoronaphthalen-2-yl)-2-((1s,4s)-4-(1-(2,6-dioxopiperidin-3-yl)-3-methyl-2-oxo-2,3-dihydro-1H-benzo[d]imidazol-5-yl)cyclohexyl)acetamide (3a) and N-(7-(benzyloxy)-6-(1,1-dioxido-4-oxo-1,2,5-thiadiazolidin-2-yl)-5-fluoronaphthalen-2-yl)-2-((1r,4r)-4-(1-(2,6-dioxopiperidin-3-yl)-3-methyl-2-oxo-2,3-dihydro-1H-benzo[d]imidazol-5-yl)cyclohexyl)acetamide (3b)

Into a 50 mL single neck round bottom flask containing a well-stirred solution of 2-[4-[1-(2,6-dioxo-3-piperidyl)-3-methyl-2-oxo-benzimidazol-5-yl]cyclohexyl]acetic acid (1, 250 mg, 538.25 μmol) in DMF (5 mL) were added EDC. HCl (309.55 mg, 1.61 mmol) and DMAP (328.79 mg, 2.69 mmol). After 15 min, 5-(6-amino-3-benzyloxy-1-fluoro-2-naphthyl)-1,1-dioxo-1,2,5-thiadiazolidin-3-one (2, 331.72 mg, 592.08 μmol, TFA salt) was added, and the reaction mixture was stirred for 16 h at 60° C. The reaction mixture was concentrated under vacuum and the residue was purified by reverse-phase preparative HPLC [Column: Silicycle-C18-120 g, Mobile phase A: 0.1% TFA in water and Mobile phase B: MeCN] to obtain N-(7-(benzyloxy)-6-(1,1-dioxido-4-oxo-1,2,5-thiadiazolidin-2-yl)-5-fluoronaphthalen-2-yl)-2-((1s,4s)-4-(1-(2,6-dioxopiperidin-3-yl)-3-methyl-2-oxo-2,3-dihydro-1H-benzo[d]imidazol-5-yl)cyclohexyl)acetamide as a mixture of stereoisomers (3a, first eluting isomer and 3b, second eluting isomer) (300 mg, 187.78 μmol, 35% yield) as an off-white solid. Compounds 3a and 3b were combined and used in Step 2. LCMS (ES+): m/z 783.0 [M+H]⁺.

### Step 2: N-(6-(1,1-dioxido-4-oxo-1,2,5-thiadiazolidin-2-yl)-5-fluoro-7-hydroxynaphthalen-2-yl)-2-((1s,4s)-4-(1-(2,6-dioxopiperidin-3-yl)-3-methyl-2-oxo-2,3-dihydro-1H-benzo[d]imidazol-5-yl)cyclohexyl)acetamide (Example 81a) and N-(6-(1,1-dioxido-4-oxo-1,2,5-thiadiazolidin-2-yl)-5-fluoro-7-hydroxynaphthalen-2-yl)-2-((1r,4r)-4-(1-(2,6-dioxopiperidin-3-yl)-3-methyl-2-oxo-2,3-dihydro-1H-benzo[d]imidazol-5-yl)cyclohexyl)acetamide (Example 81b)

Into a 100 mL single neck, round bottom flask containing a well-stirred solution of 3a and 3b (650 mg, 398.55 μmol) in anhydrous DCM (10 mL) and anhydrous toluene (10 mL) was added pentamethylbenzene (295.42 mg, 1.99 mmol) under nitrogen at RT. The reaction mixture was cooled to −78° C. and —BCl₃ (1.0 M solution in methylene chloride) (7.97 mmol, 7.97 mL) was added. The resulting mixture was stirred at RT for 3 h, cooled to −78° C. and quenched with 5% MeOH in DCM (6 mL). The volatiles were removed under reduced pressure to obtain a residue which was triturated with diethyl ether. Purification by reverse-phase preparative HPLC [Column: XSELECT-C18 (19×150) mm, 5 micron; Mobile Phase A: 10 mM Ammonium acetate in water and Mobile Phase B: MeCN] afforded first eluted fraction N-(6-(1,1-dioxido-4-oxo-1,2,5-thiadiazolidin-2-yl)-5-fluoro-7-hydroxynaphthalen-2-yl)-2-((1s,4s)-4-(1-(2,6-dioxopiperidin-3-yl)-3-methyl-2-oxo-2,3-dihydro-1H-benzo[d]imidazol-5-yl)cyclohexyl)acetamide (Example 81a, 35 mg, 47.63 μmol, 12% yield) as an off-white solid and the second eluted fraction N-(6-(1,1-dioxido-4-oxo-1,2,5-thiadiazolidin-2-yl)-5-fluoro-7-hydroxynaphthalen-2-yl)-2-((1r,4r)-4-(1-(2,6-dioxopiperidin-3-yl)-3-methyl-2-oxo-2,3-dihydro-1H-benzo[d]imidazol-5-yl)cyclohexyl)acetamide (Example 81b, 49 mg, 68.04 μmol, 17% yield) as an off-white solid. The relative stereochemistry of 81a and 81b were not determined and have been arbitrarily assigned. Example 81a: LCMS (ES+): m/z 693.0 [M+H]⁺. ¹H NMR (400 MHZ, DMSO-d₆) δ 10.10 (s, 1H), 8.15 (s, 1H), 7.80 (d, J=8.9 Hz, 1H), 7.41 (d, J=9.0 Hz, 1H), 7.09 (d, J=1.5 Hz, 1H), 6.99 (d, J=8.1 Hz, 1H), 6.94-6.88 (m, 2H), 5.33 (dd, J=12.8, 5.4 Hz, 1H), 4.06 (s, 2H), 2.97-2.82 (m, 1H), 2.77-2.58 (m, 3H), 2.54 (s, 3H), 2.34-2.26 (m, 2H), 2.05-1.95 (m, 1H), 1.94-1.78 (m, 6H), 1.64-1.46 (m, 2H), 1.27-1.11 (m, 2H)

Example 81b: LCMS (ES−): m/z 691.0 [M−H]⁻. ¹H NMR (400 MHZ, DMSO-d₆) δ 11.09 (s, 1H), 10.14 (s, 1H), 9.75 (s, 1H), 8.19-8.14 (m, 1H), 7.81 (d, J=9.0 Hz, 1H), 7.43 (dd, J=9.1, 2.0 Hz, 1H), 7.12-6.89 (m, 5H), 5.34 (dd, J=12.7, 5.5 Hz, 1H), 4.08 (s, 2H), 2.97-2.84 (m, 1H), 2.80-2.58 (m, 4H), 2.57-2.52 (m, 3H), 2.37-2.30 (m, 2H), 2.05-1.96 (m, 1H), 1.86-1.71 (m, 3H), 1.69-1.61 (m, 5H).

### 2-[[1-[1-(2,6-dioxo-3-piperidyl)-3-methyl-2-oxo-benzimidazol-5-yl]-4-hydroxy-4-piperidyl]-N-[5-fluoro-7-hydroxy-6-(1,1,4-trioxo-1,2,5-thiadiazolidin-2-yl)-2-naphthyl]acetamide (Example 82)

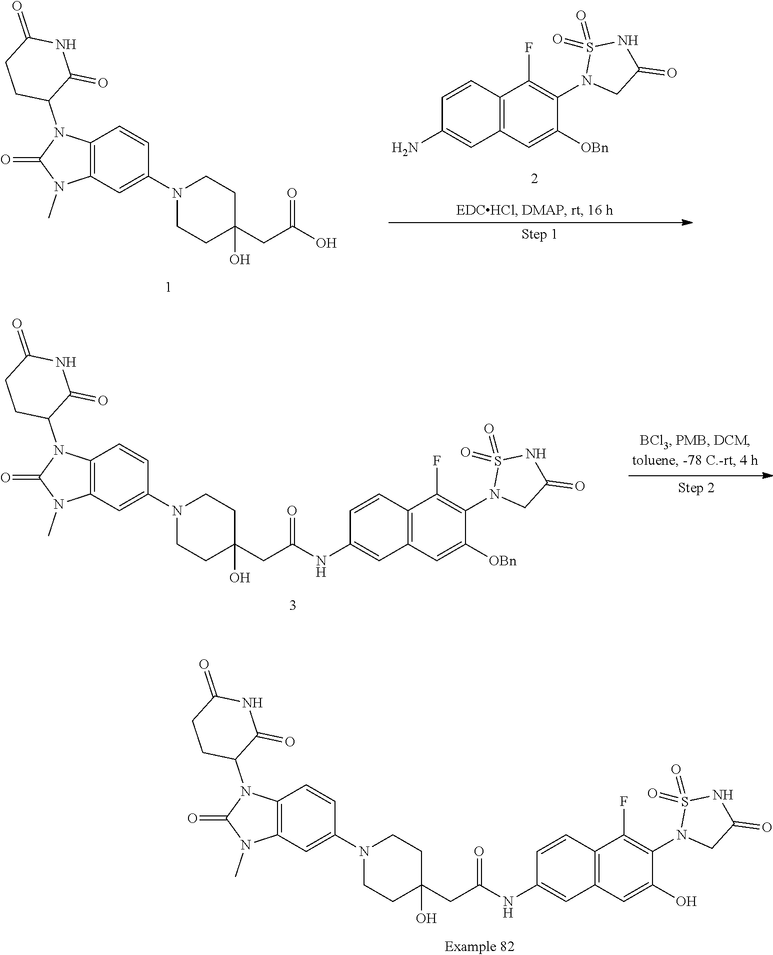 [SMILES: Cn1c(=O)n(C2CCC(=O)NC2=O)c2ccc(N3CCC(O)(CC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(O)(CC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(O)(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[1-[1-(2,6-dioxo-3-piperidyl)-3-methyl-2-oxo-benzimidazol-5-yl]-4-hydroxy-4-piperidyl]acetamide (3)

Into a 50 mL single neck round bottom flask containing a well-stirred solution of 2-[1-[1-(2,6-dioxo-3-piperidyl)-3-methyl-2-oxo-benzimidazol-5-yl]-4-hydroxy-4-piperidyl]acetic acid (1, 200 mg, 324.25 μmol, TFA salt) and 5-(6-amino-3-benzyloxy-1-fluoro-2-naphthyl)-1,1-dioxo-1,2,5-thiadiazolidin-3-one (2, 157.70 mg, 281.48 μmol, TFA salt) in anhydrous DMF (7 mL) were added EDC. HCl (124.32 mg, 648.51 μmol), 1-Hydroxybenzotriazole hydrate (74.48 mg, 486.38 μmol) and DMAP (198.07 mg, 1.62 mmol). After 16 h, the solvent was removed and the residue was treated with 1.5N HCl solution. The solid precipitate was filtered and dried under reduced pressure to afford N-[7-benzyloxy-5-fluoro-6-(1,1,4-trioxo-1,2,5-thiadiazolidin-2-yl)-2-naphthyl]-2-[[1-[1-(2,6-dioxo-3-piperidyl)-3-methyl-2-oxo-benzimidazol-5-yl]-4-hydroxy-4-piperidyl]acetamide (3, 170 mg, 74.39 μmol, 23% yield, 35% purity) as an off white color solid. The material was used in the next step without further purification. LCMS (ES+): m/z 800.3 [M+H]⁺.

### Step 2: 2-[1-[1-(2,6-dioxo-3-piperidyl)-3-methyl-2-oxo-benzimidazol-5-yl]-4-hydroxy-4-piperidyl]-N-[5-fluoro-7-hydroxy-6-(1,1,4-trioxo-1,2,5-thiadiazolidin-2-yl)-2-naphthyl]acetamide (Example 82)

Into a 50 mL single neck round bottom flask containing well-stirred solution of N-[7-benzyloxy-5-fluoro-6-(1,1,4-trioxo-1,2,5-thiadiazolidin-2-yl)-2-naphthyl]-2-[[1-[1-(2,6-dioxo-3-piperidyl)-3-methyl-2-oxo-benzimidazol-5-yl]-4-hydroxy-4-piperidyl]acetamide (3, 170 mg, 74.39 μmol, crude) and pentamethylbenzene (55.14 mg, 371.96 μmol) in anhydrous DCM (3 mL) and toluene (3 mL) was added BCl₃ solution (1.0 M in methylene chloride) (174.33 mg, 1.49 mmol, 1.49 mL) at −78° C. Then the reaction mixture was stirred at RT for 4 h. The reaction was quenched with 5% MeOH in DCM at −78° C. (3 mL). The volatiles were removed from the reaction mixture under reduced pressure and the residue was triturated with diethyl ether (50 mL) and filtered. The material was purified by reverse phase prep HPLC [Purification method: Column: X-Bridge C18 (150×19) 5 micron; Mobile phase A: 0.1% TFA in water; Mobile phase B: MeCN] to afford 2-[1-[1-(2,6-dioxo-3-piperidyl)-3-methyl-2-oxo-benzimidazol-5-yl]-4-hydroxy-4-piperidyl]-N-[5-fluoro-7-hydroxy-6-(1,1,4-trioxo-1,2,5-thiadiazolidin-2-yl)-2-naphthyl]acetamide (Example 82, 20 mg, 23.47 μmol, 32% yield, TFA salt) as an off-white solid. LCMS (ES+): m/z 710.3 [M+H]^{+ 1}H NMR (400 MHZ, DMSO-d₆) δ 11.13 (s, 1H), 10.26 (s, 1H), 10.12 (s, 1H), 8.19 (s, 1H), 7.86 (d, J=9.0 Hz, 1H), 7.46 (dd, J=9.1, 1.9 Hz, 1H), 7.22-7.12 (m, 2H), 6.96 (s, 1H), 5.37 (dd, J=12.9, 5.5 Hz, 1H), 4.20 (s, 2H), 3.38-3.35 (m, 3H), 2.95-2.83 (m, 2H), 2.74-2.57 (m, 7H), 2.25-2.10 (m, 2H), 2.05-1.86 (m, 4H).

### 2-[1-[3-(2,6-dioxo-3-piperidyl)-1-methyl-indazol-6-yl]-4-hydroxy-4-piperidyl]-N-[5-fluoro-7-hydroxy-6-(1,1,4-trioxo-1,2,5-thiadiazolidin-2-yl)-2-naphthyl]acetamide (Example 83)

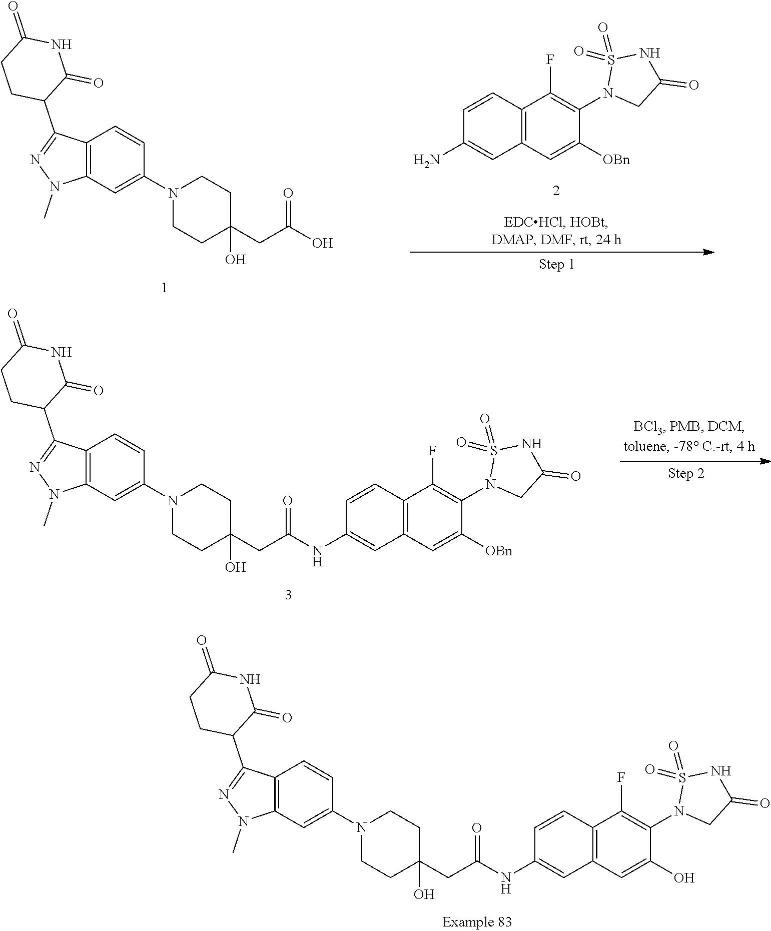 [SMILES: Cn1nc(C2CCC(=O)NC2=O)c2ccc(N3CCC(O)(CC(=O)O)CC3)cc21 ; Nc1ccc2c(F)c(N3CC(=O)NS3(=O)=O)c(OCc3ccccc3)cc2c1 ; Cn1nc(C2CCC(=O)NC2=O)c2ccc(N3CCC(O)(CC(=O)Nc4ccc5c(F)c(N6CC(=O)NS6(=O)=O)c(OCc6ccccc6)cc5c4)CC3)cc21 ; Cn1nc(C2CCC(=O)NC2=O)c2ccc(N3CCC(O)(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[1-[3-(2,6-dioxo-3-piperidyl)-1-methyl-indazol-6-yl]-4-hydroxy-4-piperidyl]acetamide (3)

Into a 10 mL single neck round bottom flask containing well-stirred solution of 2-(1-(3-(2,6-dioxopiperidin-3-yl)-1-methyl-1H-indazol-6-yl)-4-hydroxypiperidin-4-yl)acetic acid (1, 200 mg, 499.47 μmol) and 5-(6-amino-3-benzyloxy-1-fluoro-2-naphthyl)-1,1-dioxo-1,2,5-thiadiazolidin-3-one (2, 268.17 mg, 499.47 μmol, TFA salt) in anhydrous DMF (8 mL) were added EDC. HCl (287.24 mg, 1.50 mmol), DMAP (366.11 mg, 3.00 mmol) and HOBt (134.98 mg, 998.93 μmol). After 24 h, the solvent was removed, and the residue was treated with 1.5 N aqueous HCl (10 mL). The precipitate was filtered and dried under reduced pressure to afford N-[7-benzyloxy-5-fluoro-6-(1,1,4-trioxo-1,2,5-thiadiazolidin-2-yl)-2-naphthyl]-2-[1-[3-(2,6-dioxo-3-piperidyl)-1-methyl-indazol-6-yl]-4-hydroxy-4-piperidyl]acetamide (3, 220 mg, 93.87 μmol, 19% yield, 35% purity, HCl salt) as off white solid. LCMS (ES+): m/z 784.2 [M+H]⁺.

### Step 2: 2-[1-[3-(2,6-dioxo-3-piperidyl)-1-methyl-indazol-6-yl]-4-hydroxy-4-piperidyl]-N-[5-fluoro-7-hydroxy-6-(1,1,4-trioxo-1,2,5-thiadiazolidin-2-yl)-2-naphthyl]acetamide (Example 83)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[1-[3-(2,6-dioxo-3-piperidyl)-1-methyl-indazol-6-yl]-4-hydroxy-4-piperidyl]acetamide (3, 220 mg, 93.87 μmol, HCl salt, crude) and pentamethylbenzene (69.58 mg, 469.35 μmol) in anhydrous DCM (3 mL) and toluene (3 mL) was added BCl₃ solution (1.0 M in methylene chloride) (219.97 mg, 1.88 mmol, 1.88 mL) at −78° C. The reaction mixture was stirred at RT. After 4 h, the reaction mixture was quenched with 5% MeOH in DCM (3 mL) at −78° C. The volatiles were removed under reduced pressure and the residue was triturated with diethyl ether (50 mL) and filtered. The material was purified by reverse phase prep HPLC [Purification method: Column: X-select C18 (19×150) mm 5 micron; Mobile phase: 0.1% TFA in water; Mobile phase B: MeCN] to afford 2-[1-[3-(2,6-dioxo-3-piperidyl)-1-methyl-indazol-6-yl]-4-hydroxy-4-piperidyl]-N-[5-fluoro-7-hydroxy-6-(1,1,4-trioxo-1,2,5-thiadiazolidin-2-yl)-2-naphthyl]acetamide (Example 83, 45 mg, 53.28 μmol, 57% yield, TFA salt) as an off-white solid. LCMS (ES+): m/z 694.2 [M+H]^{+ 1}H NMR (400 MHZ, DMSO-d₆) δ 10.87 (s, 1H), 10.26 (s, 1H), 10.17 (s, 1H), 8.22-8.16 (m, 1H), 7.85 (d, J=8.9 Hz, 1H), 7.56 (d, J=9.0 Hz, 1H), 7.44 (dd, J=9.1, 1.9 Hz, 1H), 7.16 (d, J=51.1 Hz, 1H), 7.01 (d, J=9.0 Hz, 1H), 6.98-6.95 (m, 1H), 4.33-4.25 (m, 3H), 3.91 (s, 3H), 2.71-2.54 (m, 6H), 2.36-2.25 (m, 1H), 2.20-2.10 (m, 1H), 2.02-1.89 (m, 2H), 1.84-1.73 (m, 2H).

### 2-[[1-[1-(2,6-dioxo-3-piperidyl)-3-methyl-2-oxo-benzimidazol-5-yl]-4-piperidyl]oxy]-N-[5-fluoro-7-hydroxy-6-(1,1,4-trioxo-1,2,5-thiadiazolidin-2-yl)-2-naphthyl]acetamide (Example 84)

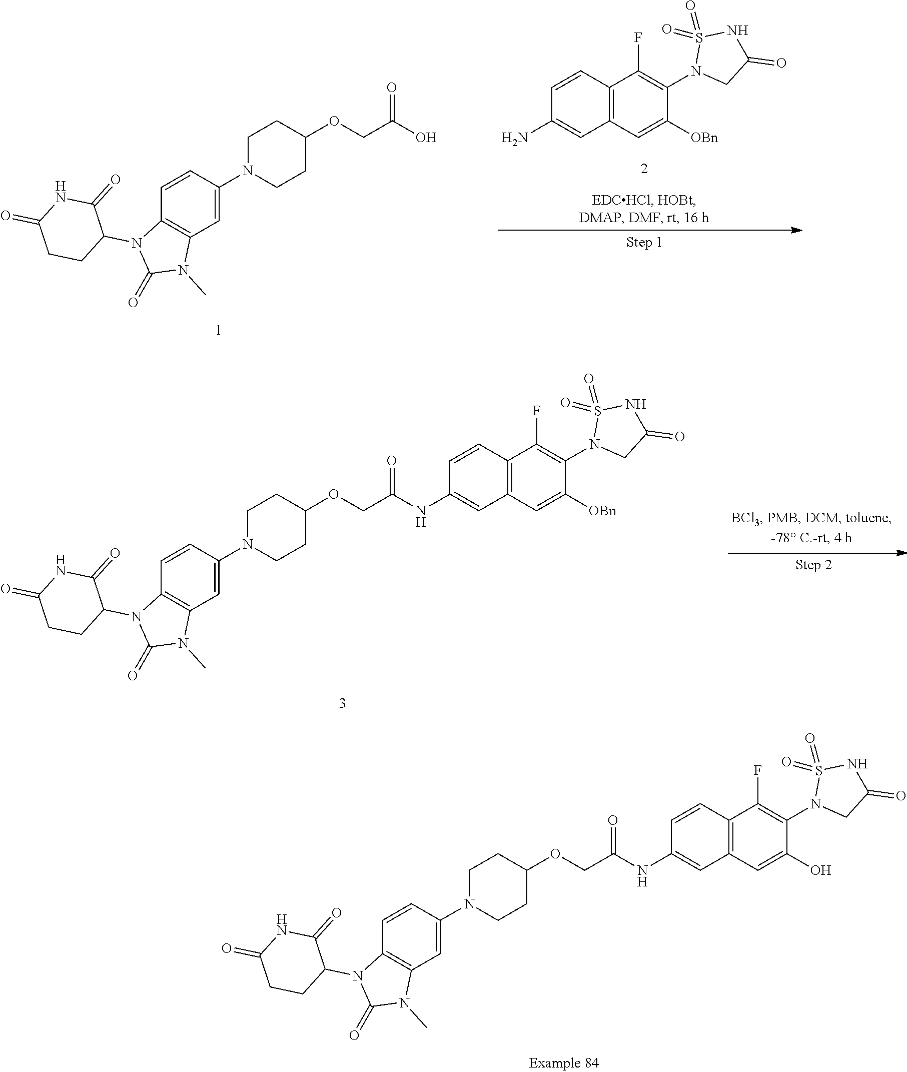 [SMILES: Cn1c(=O)n(C2CCC(=O)NC2=O)c2ccc(N3CCC(OCC(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(OCC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(OCC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[[1-[1-(2,6-dioxo-3-piperidyl)-3-methyl-2-oxo-benzimidazol-5-yl]-4-piperidyl]oxy]acetamide (3)

Into a 20 mL vial containing a well-stirred solution of 2-[[1-[1-(2,6-dioxo-3-piperidyl)-3-methyl-2-oxo-benzimidazol-5-yl]-4-piperidyl]oxy]acetic acid (1, 170 mg, 301.25 μmol, TFA salt) in DMF (2.5 mL) was added 5-(6-amino-3-benzyloxy-1-fluoro-2-naphthyl)-1,1-dioxo-1,2,5-thiadiazolidin-3-one (2, 161.75 mg, 301.25 μmol, TFA salt), N-(3-dimethylaminopropyl)-N′-ethylcarbodiimide hydrochloride (173.25 mg, 903.76 μmol), hydroxy benzotriazole (81.41 mg, 602.51 μmol) and 4-dimethylaminopyridine (220.83 mg, 1.81 mmol). After 16 h, the reaction mixture was concentrated under vacuum. The residue was treated with 1.5 N HCl (10 mL), the precipitate was filtered and washed with water (20 mL) and diethyl ether (20 mL) and dried under vacuum to afford N-[7-benzyloxy-5-fluoro-6-(1,1,4-trioxo-1,2,5-thiadiazolidin-2-yl)-2-naphthyl]-2-[[1-[1-(2,6-dioxo-3-piperidyl)-3-methyl-2-oxo-benzimidazol-5-yl]-4-piperidyl]oxy]acetamide (3, 230 mg, 221.42 μmol, 74% yield) as an brown solid. LCMS (ES+): m/z 800.2 [M+H]⁺.

### Step 2: 2-[[1-[1-(2,6-dioxo-3-piperidyl)-3-methyl-2-oxo-benzimidazol-5-yl]-4-piperidyl]oxy]-N-[5-fluoro-7-hydroxy-6-(1,1,4-trioxo-1,2,5-thiadiazolidin-2-yl)-2-naphthyl]acetamide (Example 84)

Into a 50 mL round bottom flask containing a well-stirred solution of N-[7-benzyloxy-5-fluoro-6-(1,1,4-trioxo-1,2,5-thiadiazolidin-2-yl)-2-naphthyl]-2-[[1-[1-(2,6-dioxo-3-piperidyl)-3-methyl-2-oxo-benzimidazol-5-yl]-4-piperidyl]oxy]acetamide (3, 230 mg, 221.42 μmol) in DCM (2 mL) and toluene (2 mL) was added pentamethylbenzene (164.13 mg, 1.11 mmol, 178.98 μL) and the suspension was cooled to −78° C. BCl₃ solution (1.0 M in DCM) (518.89 mg, 4.43 mmol, 0.44 mL) was added dropwise and the reaction mixture was brought to RT. After 4 h, the reaction mixture was cooled to −78° C. and quenched slowly with 5% methanol in DCM (2 mL). The reaction mixture was concentrated under reduced pressure and the residue was triturated with diethyl ether (10 mL), filtered and dried. The material was purified by reverse phase prep HPLC [Purification method: Column: X-Bridge, C18 (150×19) mm, 5 micron; Mobile phase A: 0.1% TFA in water; Mobile phase B: MeCN] to afford 2-[[1-[1-(2,6-dioxo-3-piperidyl)-3-methyl-2-oxo-benzimidazol-5-yl]-4-piperidyl]oxy]-N-[5-fluoro-7-hydroxy-6-(1,1,4-trioxo-1,2,5-thiadiazolidin-2-yl)-2-naphthyl]acetamide (Example 84, 33 mg, 39.18 μmol, 18% yield, TFA Salt) as a colorless solid. LCMS (ES+): m/z 710.0 [M+H]^{+ 1}H NMR (400 MHZ, DMSO-d₆) δ 11.11 (s, 1H), 10.19 (s, 1H), 9.93 (s, 1H), 8.20-8.14 (m, 1H), 7.87 (d, J=9.0 Hz, 1H), 7.56 (dd, J=9.1, 2.0 Hz, 1H), 7.27 (s, 1H), 7.17-6.92 (m, 3H), 5.36 (dd, J=12.8, 5.4 Hz, 1H), 4.27 (s, 2H), 4.22 (s, 2H), 3.77 (s, 2H), 3.64 (s, 3H), 2.97-2.83 (m, 2H), 2.77-2.56 (m, 3H), 2.20-2.11 (m, 2H), 2.05-1.84 (m, 3H).

### Preparation of Further Exemplary Formula (I) Compounds

### 2-[[7-benzyloxy-5-fluoro-6-(1,1,4-trioxo-1,2,5-thiadiazolidin-2-yl)-2-naphthyl]amino]-2-oxo-acetic acid (4)

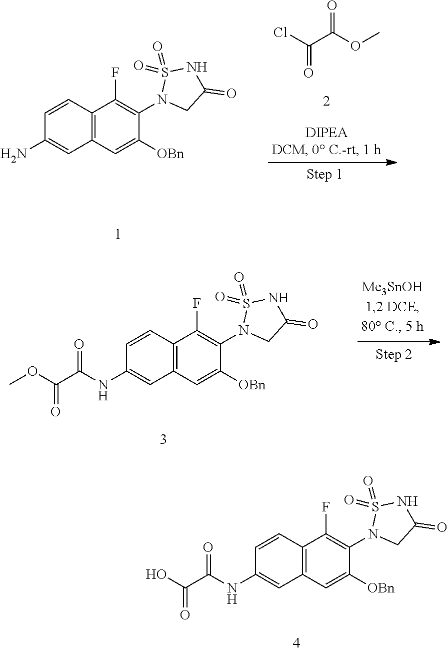

### Step 1: methyl 2-[[7-benzyloxy-5-fluoro-6-(1,1,4-trioxo-1,2,5-thiadiazolidin-2-yl)-2-naphthyl]amino]-2-oxo-acetate (3)

Into a 50 mL single neck round bottom flask containing a well-stirred solution of 5-(6-amino-3-benzyloxy-1-fluoro-2-naphthyl)-1,1-dioxo-1,2,5-thiadiazolidin-3-one (1, 500 mg, 920.87 μmol, TFA salt) in anhydrous DCM (4 mL) was added DIPEA (357.05 mg, 2.76 mmol, 481.2 μL). The mixture was cooled to 0° C. and methyl 2-chloro-2-oxo-acetate (1 M in anhydrous DCM) (2, 112.81 mg, 920.87 μmol, 0.92 mL) was added. The reaction mixture was stirred at RT for 1 h. The reaction mixture was diluted with water (15 mL) and extracted with DCM (25 mL×2). The organic layer was washed with brine (25 mL) and dried over anhydrous sodium sulfate. The solvent was evaporated under reduced pressure and the residue was purified by flash silica gel (230-400 mesh) column chromatography (5% MeOH in DCM) to afford methyl 2-[[7-benzyloxy-5-fluoro-6-(1,1,4-trioxo-1,2,5-thiadiazolidin-2-yl)-2-naphthyl]amino]-2-oxo-acetate (3, 470 mg, 97% yield) as a colorless solid. LCMS (ES+): m/z 488.0 [M+H]⁺

### Step 2: 2-[[7-benzyloxy-5-fluoro-6-(1,1,4-trioxo-1,2,5-thiadiazolidin-2-yl)-2-naphthyl]amino]-2-oxo-acetic acid (4)

Into a 25 mL single neck round bottom flask containing a well-stirred solution of methyl 2-[[7-benzyloxy-5-fluoro-6-(1,1,4-trioxo-1,2,5-thiadiazolidin-2-yl)-2-naphthyl]amino]-2-oxo-acetate (3, 470 mg, 887.05 μmol) in 1,2-dichloroethane (5 mL) was added trimethyltin hydroxide (801.99 mg, 44.4 mmol) at RT and the suspension was stirred for 5 h at 80° C. The volatiles were removed under reduced pressure and the residue was purified by reverse phase column chromatography [Method: Silicycle 120 g (C18) 25 micron column; Mobile phase A: 0.1% formic acid in water; Mobile phase B: Acetonitrile] to afford 2-[[7-benzyloxy-5-fluoro-6-(1,1,4-trioxo-1,2,5-thiadiazolidin-2-yl)-2-naphthyl]amino]-2-oxo-acetic acid (4, 450 mg, 90% yield) as a brown solid. LCMS (ES−): m/z 472.0 [M−H]⁻

### 5-[3-benzyloxy-7-(2,5-dihydro-1H-pyrrol-3-yl)-1-fluoro-2-naphthyl]-1,1-dioxo-1,2,5-thiadiazolidin-3-one (4)

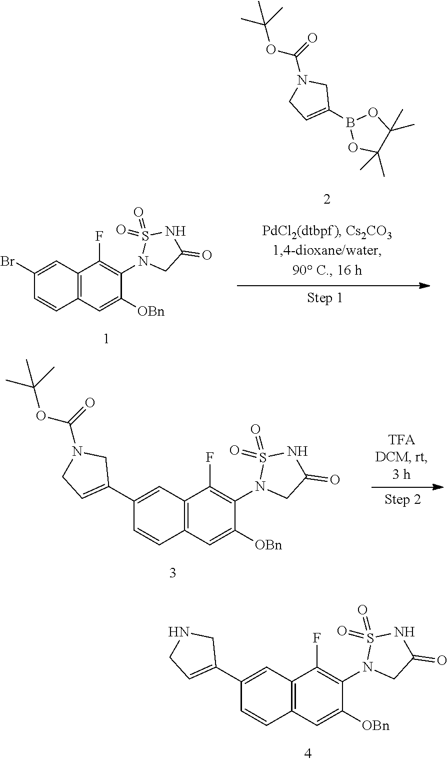 [SMILES: CC(C)(C)OC(=O)N1CC=C(B2OC(C)(C)C(C)(C)O2)C1 ; O=C1CN(c2c(OCc3ccccc3)cc3ccc(Br)cc3c2F)S(=O)(=O)N1 ; CC(C)(C)OC(=O)N1CC=C(c2ccc3cc(OCc4ccccc4)c(N4CC(=O)NS4(=O)=O)c(F)c3c2)C1 ; O=C1CN(c2c(OCc3ccccc3)cc3ccc(C4=CCNC4)cc3c2F)S(=O)(=O)N1]

### Step 1: tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3)

Into a 250 mL sealed tube containing a well-stirred solution of 5-(3-benzyloxy-7-bromo-1-fluoro-2-naphthyl)-1,1-dioxo-1,2,5-thiadiazolidin-3-one (1, 3 g, 6.45 mmol) and tert-butyl 3-(4,4,5,5-tetramethyl-1,3,2-dioxaborolan-2-yl)-2,5-dihydropyrrole-1-carboxy late (2, 1.90 g, 6.45 mmol) in anhydrous 1,4-dioxane (43 mL) and water (17 mL) were added cesium carbonate (10.50 g, 32.24 mmol) and PdCl₂(dtbpf) (336.18 mg, 515.81 μmol). The reaction mixture was degassed by purging nitrogen gas for 15 min. Then, the reaction mixture was stirred at 90° C. for 16 h. The reaction mixture was quenched with water (75 mL). The aqueous layer was extracted with EtOAc (4×40 mL). The combined organic layer was dried over anhydrous Na₂SO₄, filtered and concentrated under reduced pressure to afford tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3, 3.1 g, 4.84 mmol, 75% yield) as brown solid. LCMS (ES+): m/z 454.1 [M-Boc+H]⁺

### Step 2: 5-[3-benzyloxy-7-(2,5-dihydro-1H-pyrrol-3-yl)-1-fluoro-2-naphthyl]-1,1-dioxo-1,2,5-thiadiazolidin-3-one (4)

Into a 50 mL single neck round bottom flask containing a well-stirred solution of tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (3, 1 g, 1.56 mmol) in anhydrous DCM (7 mL) was added TFA (4.44 g, 38.94 mmol, 3 mL). After 3 h the solvent was removed from the reaction mixture under reduced pressure. The residue was purified by washing with diethyl ether (100 mL) to obtain 5-[3-benzyloxy-7-(2,5-dihydro-1H-pyrrol-3-yl)-1-fluoro-2-naphthyl]-1,1-dioxo-1,2,5-thiadiazolidin-3-one (4, 700 mg, 1.15 mmol, 74% yield, TFA salt) as an off-white solid. LCMS (ES+): m/z 454.2 [M+H]⁺

### 3-[4-[6-benzyloxy-8-fluoro-7-(1,1,4-trioxo-1,2,5-thiadiazolidin-2-yl)-2-naphthyl]pyrazol-1-yl]propanal (6)

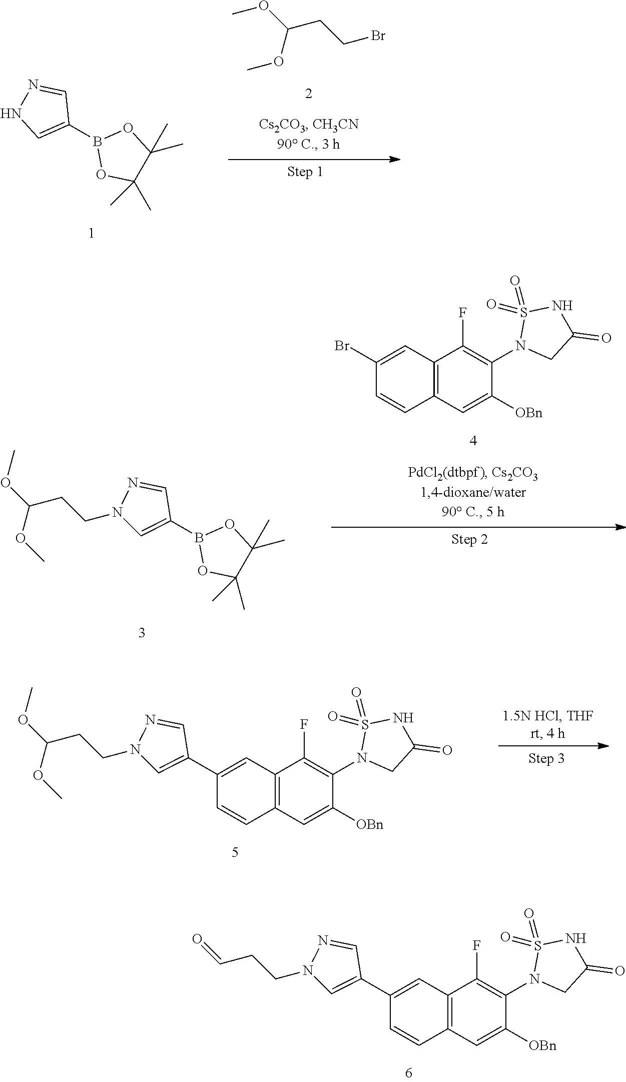

### Step 1: 1-(3,3-dimethoxypropyl)-4-(4,4,5,5-tetramethyl-1,3,2-dioxaborolan-2-yl)pyrazole (3)

Into a 50 mL single neck round bottom flask containing a well-stirred solution of 4-(4,4,5,5-tetramethyl-1,3,2-dioxaborolan-2-yl)-1H-pyrazole (1, 500 mg, 2.58 mmol) in acetonitrile (10 mL) were added 3-bromo-1,1-dimethoxy-propane (2, 566.00 mg, 3.09 mmol, 416.18 μL) and cesium carbonate (1.68 g, 5.15 mmol). The resultant reaction mixture was stirred at 90° C. for 3 h. The reaction mixture was diluted with EtOAc (50 mL), filtered through celite, washing with ethyl acetate (10 mL). The filtrate was washed with water (25 mL×2) and brine solution (25 mL) and dried over anhydrous Na₂SO₄. The solvent was removed to afford 1-(3,3-dimethoxypropyl)-4-(4,4,5,5-tetramethyl-1,3,2-dioxaborolan-2-yl)pyrazole 3 (600 mg, 67% yield) as a colorless liquid. The material was taken to next step without purification. LCMS (ES+): m/z 297.2 [M+H]⁺

### Step 2: 5-(3-(benzyloxy)-7-(1-(3,3-dimethoxypropyl)-1H-pyrazol-4-yl)-1-fluoronaphthalen-2-yl)-1,2,5-thiadiazolidin-3-one 1,1-dioxide (5)

Into a 20 mL pressure tube containing a well-stirred solution of 5-(3-benzyloxy-7-bromo-1-fluoro-2-naphthyl)-1,1-dioxo-1,2,5-thiadiazolidin-3-one (4, 300 mg, 644.76 μmol) and 1-(3,3-dimethoxypropyl)-4-(4,4,5,5-tetramethyl-1,3,2-dioxaborolan-2-yl)pyrazole (3, 229.15 mg, 773.71 μmol) in anhydrous 1,4-dioxane (8.0 mL) and water (2.0 mL) was added cesium carbonate (630.22 mg, 1.93 mmol). The reaction mixture was purged with nitrogen gas for 10 min. Then PdCl₂(dtbpf) (25.21 mg, 38.69 μmol) was added. The reaction mixture was heated to 90° C. for 5 h. The reaction mixture was cooled to RT, filtered through celite and washed with ethyl acetate (20 mL). The filtrate was washed with water (15 mL), and aqueous layer was extracted with 10% MeOH in EtOAc (2×50 mL). The combined organic layer was washed with brine solution (15 mL) and dried over anhydrous Na₂SO₄. The solvent was evaporated under reduced pressure. The residue was purified by flash silica gel column chromatography (50 g, 60-120 mesh) using 10-15% MeOH in DCM to afford 5-(3-(benzyloxy)-7-(1-(3,3-dimethoxypropyl)-1H-pyrazol-4-yl)-1-fluoronaphthalen-2-yl)-1,2,5-thiadiazolidin-3-one 1,1-dioxide 5 (275 mg, 70% yield) as a brown solid. LCMS (ES+): m/z 555.5 [M+H]⁺

### Step 3: 3-[4-[6-benzyloxy-8-fluoro-7-(1,1,4-trioxo-1,2,5-thiadiazolidin-2-yl)-2-naphthyl]pyrazol-1-yl]propanal (6)

Into a 25 mL single neck round bottom flask containing a well-stirred solution of 5-[3-benzyloxy-7-[1-(3,3-dimethoxypropyl)pyrazol-4-yl]-1-fluoro-2-naphthyl]-1,1-dioxo-1,2,5-thiadiazolidin-3-one 5 (275 mg, 495.86 μmol) in THF (2.0 mL) was added a aqueous solution of 1.5 N HCl (2.75 g, 75.43 mmol) at (° C.) The resultant mixture was stirred at RT for 4 h. The volatiles were removed and co-distilled with toluene (2×2 mL). The residue was triturated with diethyl ether (5 mL), filtered and dried to afford 3-[4-[6-benzyloxy-8-fluoro-7-(1,1,4-trioxo-1,2,5-thiadiazolidin-2-yl)-2-naphthyl]pyrazol-1-yl]propanal (6, 245 mg, 97% yield) as a brown solid. The material was used immediately in the next step without further characterization.

### 5-[[6-benzyloxy-8-fluoro-7-(1,1,4-trioxo-1,2,5-thiadiazolidin-2-yl)-2-naphthyl]oxy]pentanal (3)

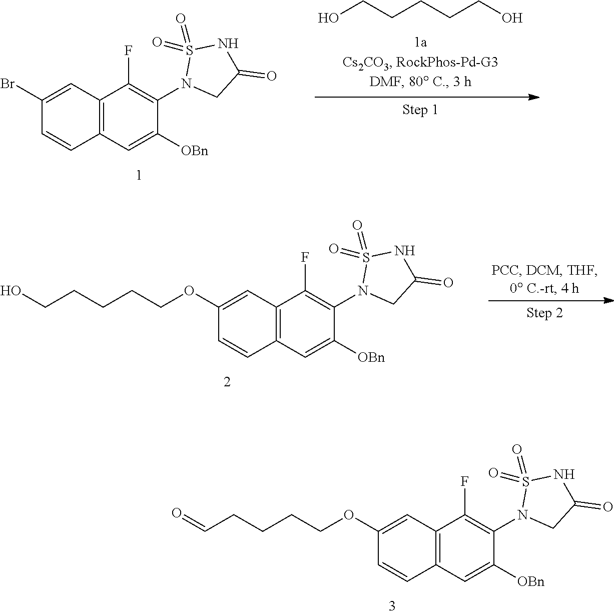 [SMILES: OCCCCCO ; O=C1CN(c2c(OCc3ccccc3)cc3ccc(Br)cc3c2F)S(=O)(=O)N1 ; O=C1CN(c2c(OCc3ccccc3)cc3ccc(OCCCCCO)cc3c2F)S(=O)(=O)N1 ; O=CCCCCOc1ccc2cc(OCc3ccccc3)c(N3CC(=O)NS3(=O)=O)c(F)c2c1]

### Step 1: 5-[3-benzyloxy-1-fluoro-7-(5-hydroxypentoxy)-2-naphthyl]-1,1-dioxo-1,2,5-thiadiazolidin-3-one (2)

Into a 10 mL pressure tube containing a well-stirred solution of 5-(3-benzyloxy-7-bromo-1-fluoro-2-naphthyl)-1,1-dioxo-1,2,5-thiadiazolidin-3-one (1, 500 mg, 1.07 mmol) and pentane-1,5-diol (1.12 g, 10.75 mmol, 1.13 mL) in anhydrous DMF (2.5 mL) were added cesium carbonate (700.25 mg, 2.15 mmol) and RockPhos Pd G3 (18.02 mg, 21.49 μmol) at RT. The mixture was purged with nitrogen gas for 15 min. The tube was sealed and heated at 90° C. for 3 h. The reaction mixture was filtered through Celite, filtrate was concentrated under reduced pressure and the residue was purified by reverse phase column chromatography [Purification method: Biotage C18 column; Mobile phase A: 0.1% TFA in water; Mobile phase B: MeCN) to afford 5-[3-benzyloxy-1-fluoro-7-(5-hydroxypentoxy)-2-naphthyl]-1,1-dioxo-1,2,5-thiadiazolidin-3-one (2, 200 mg, 388.80 μmol, 36% yield) as an off-white solid. LCMS (ES+): m/z 489.2 [M+H]⁺

### Step 2: 5-[[6-benzyloxy-8-fluoro-7-(1,1,4-trioxo-1,2,5-thiadiazolidin-2-yl)-2-naphthyl]oxy]pentanal (3)

Into a 25 mL single neck round bottom flask containing a well-stirred solution of 5-[3-benzyloxy-1-fluoro-7-(5-hydroxypentoxy)-2-naphthyl]-1,1-dioxo-1,2,5-thiadiazolidin-3-one (2, 170 mg, 330.59 μmol) in anhydrous THF (3 mL) and DCM (5 mL) was added PCC (121.14 mg, 561.99 μmol) at RT. The reaction mixture was stirred at RT for 4 h. The reaction mixture was filtered through Celite, filtrate was concentrated to afford 5-[[6-benzyloxy-8-fluoro-7-(1,1,4-trioxo-1,2,5-thiadiazolidin-2-yl)-2-naphthyl]oxy]pentanal (3, 250 mg, 157.76 μmol, 48% yield, 31% purity, crude) as brown solid. This material was taken to next step without purification. LCMS (ES−): m/z 485.2 [M−H]⁻

### 5-[3-benzyloxy-1-fluoro-7-[2-(methylamino)ethoxy]-2-naphthyl]-1,1-dioxo-1,2,5-thiadiazolidin-3-one (4)

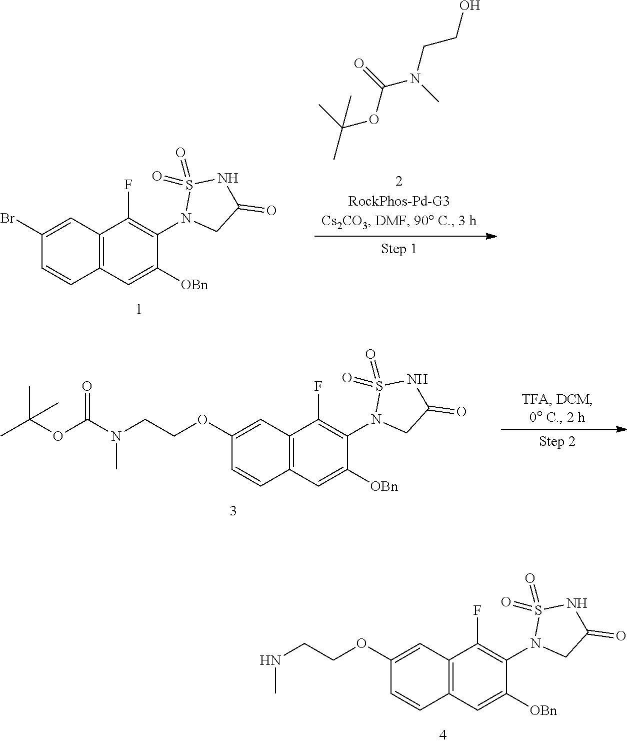 [SMILES: O=C1CN(c2c(OCc3ccccc3)cc3ccc(Br)cc3c2F)S(=O)(=O)N1 ; CN(CCO)C(=O)OC(C)(C)C ; CN(CCOc1ccc2cc(OCc3ccccc3)c(N3CC(=O)NS3(=O)=O)c(F)c2c1)C(=O)OC(C)(C)C ; CNCCOc1ccc2cc(OCc3ccccc3)c(N3CC(=O)NS3(=O)=O)c(F)c2c1]

### Step 1: tert-butyl N-[2-[[6-benzyloxy-8-fluoro-7-(1,1,4-trioxo-1,2,5-thiadiazolidin-2-yl)-2-naphthyl]oxy]ethyl]-N-methyl-carbamate (3)

Into a 25 mL pressure tube containing a well-stirred solution of tert-butyl N-(2-hydroxyethyl)-N-methyl-carbamate (2, 753.18 mg, 4.30 mmol, 24.98 μL) in DMF (5 mL) were added 5-(3-benzyloxy-7-bromo-1-fluoro-2-naphthyl)-1,1-dioxo-1,2,5-thiadiazolidin-3-one (1, 500 mg, 1.07 mmol) and cesium carbonate (700.25 mg, 2.15 mmol). The mixture was degassed by bubbling nitrogen gas through for 5 min. Then RockPhos Pd G3 (27.03 mg, 32.24 μmol) was added and degassed for another 5 min. The reaction mixture was stirred at 90° C. After 3 h, the reaction mixture was filtered through Celite, washing with ethyl acetate (20 mL). The filtrate was concentrated under reduced pressure and the residue subjected to reverse phase column chromatography [Purification method: Column: Siliasep premium C18, 25 μm, 120 g; Mobile phase A: 0.1% TFA in water; Mobile phase B: Acetonitrile) to afford tert-butyl N-[2-[[6-benzyloxy-8-fluoro-7-(1,1,4-trioxo-1,2,5-thiadiazolidin-2-yl)-2-naphthyl]oxy]ethyl]-N-methyl-carbamate (3, 200 mg, 308.22 μmol, 29% yield) as a pale yellow solid. LCMS (ES+): m/z 460.0 [M-Boc+H]⁺

### Step 2: 5-[3-benzyloxy-1-fluoro-7-[2-(methylamino)ethoxy]-2-naphthyl]-1,1-dioxo-1,2,5-thiadiazolidin-3-one (4)

Into a 25 mL single neck round bottom flask containing a well-stirred solution of tert-butyl N-[2-[6-benzyloxy-8-fluoro-7-(1,1,4-trioxo-1,2,5-thiadiazolidin-2-yl)-2-naphthyl]oxy]ethyl]-N-methyl-carbamate (3, 200 mg, 357.39 μmol) in DCM (2 mL) was added trifluoroacetic acid (1.04 g, 9.09 mmol, 0.7 mL) at 0° C. After 2 h at RT, the volatiles were removed under reduced pressure and the residue was triturated with diethyl ether (2×7 mL), filtered and dried to afford 5-[3-benzyloxy-1-fluoro-7-[2-(methylamino)ethoxy]-2-naphthyl]-1,1-dioxo-1,2,5-thiadiazolidin-3-one (4,180 mg, 278.70 μmol, 78% yield, TFA salt) as a pale yellow solid. LCMS (ES+): m/z 460.2 [M+H]⁺

### 6-[[6-benzyloxy-8-fluoro-7-(1,1,4-trioxo-1,2,5-thiadiazolidin-2-yl)-2-naphthyl]oxy]hexanal (3)

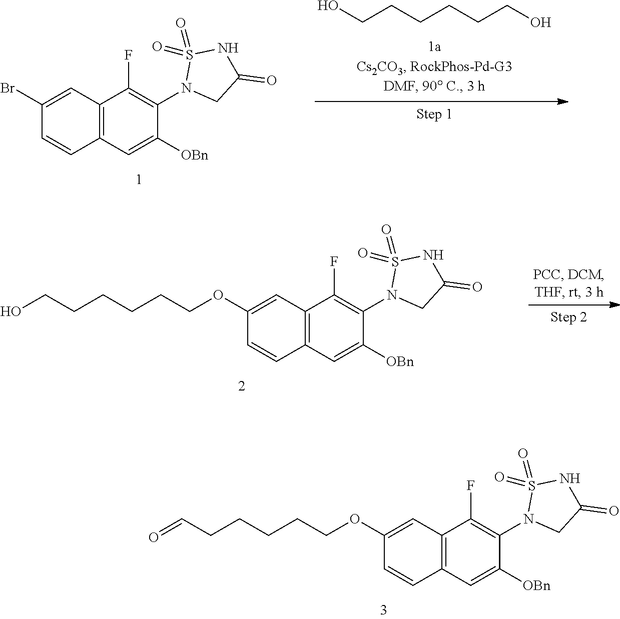 [SMILES: OCCCCCCO ; O=C1CN(c2c(OCc3ccccc3)cc3ccc(Br)cc3c2F)S(=O)(=O)N1 ; O=C1CN(c2c(OCc3ccccc3)cc3ccc(OCCCCCCO)cc3c2F)S(=O)(=O)N1 ; O=CCCCCCOc1ccc2cc(OCc3ccccc3)c(N3CC(=O)NS3(=O)=O)c(F)c2c1]

### Step 1: 5-[3-benzyloxy-1-fluoro-7-(6-hydroxyhexoxy)-2-naphthyl]-1,1-dioxo-1,2,5-thiadiazolidin-3-one (2)

Into a 50 mL pressure tube containing a well-stirred solution of 5-(3-benzyloxy-7-bromo-1-fluoro-2-naphthyl)-1,1-dioxo-1,2,5-thiadiazolidin-3-one (1, 500 mg, 1.07 mmol) in anhydrous DMF (10 mL) were added cesium carbonate (875.31 mg, 2.69 mmol) and hexane-1,6-diol (1a, 507.95 mg, 4.30 mmol, 453.53 μL). The solution was degassed by bubbling nitrogen gas through for 5 min. Then RockPhos Pd G3 (45.05 mg, 53.73 μmol) was added. The reaction was heated at 90° C. for 2 h. The reaction mixture was filtered through a pad of Celite. The filtrate was evaporated under reduced pressure and the residue purified by reverse phase column chromatography [Purification method: Siliasep premium C-18, 25 μm, 120 g; Mobile phase A: 0.1% TFA in water; Mobile phase B: Acetonitrile] to afford 5-[3-benzyloxy-1-fluoro-7-(6-hydroxyhexoxy)-2-naphthyl]-1,1-dioxo-1,2,5-thiadiazolidin-3-one (200 mg, 392.71 μmol, 37% yield) as an off-white solid. LCMS (ES+): m/z 503.2 [M+H]⁺

### Step 2: 6-[[6-benzyloxy-8-fluoro-7-(1,1,4-trioxo-1,2,5-thiadiazolidin-2-yl)-2-naphthyl]oxy]hexanal (3)

Into a 50 mL single neck round bottom flask containing a well-stirred solution of 5-[3-benzyloxy-1-fluoro-7-(6-hydroxyhexoxy)-2-naphthyl]-1,1-dioxo-1,2,5-thiadiazolidin-3-one (2, 200 mg, 392.75 μmol) in anhydrous DCM (3 mL) and anhydrous THF (1 mL) was added pyridinium chlorochromate (126.99 mg, 589.13 μmol) at 0° C. and stirred at RT for 3 h. The reaction mixture was filtered through Celite and washed with DCM (20 mL). The filtrate was evaporated under reduced pressure and the residue triturated with Et₂O to afford 6-[[6-benzyloxy-8-fluoro-7-(1,1,4-trioxo-1,2,5-thiadiazolidin-2-yl)-2-naphthyl]oxy]hexanal (3, 300 mg, 359.61 μmol, 92% yield, 60% purity) as a dark grey solid. LCMS (ES−): m/z 499.2 [M−H]⁻

### 2-[[6-benzyloxy-8-fluoro-7-(1,1,4-trioxo-1,2,5-thiadiazolidin-2-yl)-2-naphthyl]oxy]acetic acid (4)

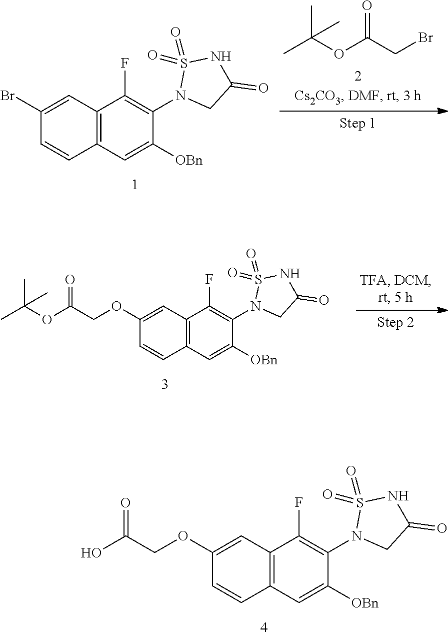

### Step 1: tert-butyl 2-[[6-benzyloxy-8-fluoro-7-(1,1,4-trioxo-1,2,5-thiadiazolidin-2-yl)-2-naphthyl]oxy]acetate (3)

Into a 50 mL single neck round bottom flask containing well-stirred solution of 5-(3-benzyloxy-1-fluoro-7-hydroxy-2-naphthyl)-1,1-dioxo-1,2,5-thiadiazolidin-3-one (1, 350 mg, 869.79 μmol) in DMF (5 mL) were added tert-butyl 2-bromoacetate (2, 169.66 mg, 869.79 μmol, 127.56 μL) and cesium carbonate (566.79 mg, 1.74 mmol). After 2 h, water (25 mL) was added to the reaction mixture and extracted with EtOAc (3×50 mL). The combined organic layer was dried over anhydrous Na₂SO₄, filtered and concentrated under reduced pressure. The residue was purified by reverse phase column chromatography [Purification method: Biotage C18 column; Mobile phase: 0.1% Formic Acid in water; Mobile phase B: MeCN] to obtain tert-butyl 2-[[6-benzyloxy-8-fluoro-7-(1,1,4-trioxo-1,2,5-thiadiazolidin-2-yl)-2-naphthyl]oxy]acetate (3, 250 mg, 480.99 μmol, 55% yield) as an off white solid. LCMS (ES−): m/z 515.2 [M−H]⁻

### Step 2: 2-[[6-benzyloxy-8-fluoro-7-(1,1,4-trioxo-1,2,5-thiadiazolidin-2-yl)-2-naphthyl]oxy]acetic acid (4)

Into a 50 mL single neck round bottom flask containing a well-stirred solution of tert-butyl 2-[[6-benzyloxy-8-fluoro-7-(1,1,4-trioxo-1,2,5-thiadiazolidin-2-yl)-2-naphthyl]oxy]acetate (3, 250.00 mg, 480.99 μmol) in anhydrous DCM (6 mL) was added TFA (2.22 g, 19.47 mmol, 1.5 mL). After 5 h, the reaction mixture was concentrated and the residue was washed with diethyl ether (100 mL) to afford 2-[[6-benzyloxy-8-fluoro-7-(1,1,4-trioxo-1,2,5-thiadiazolidin-2-yl)-2-naphthyl]oxy]acetic acid (4, 200 mg, 420.82 μmol, 87% yield) as an off-white solid. LCMS (ES−): m/z 459.0 [M−H]⁻

### 5-[[6-benzyloxy-8-fluoro-7-(1,1,4-trioxo-1,2,5-thiadiazolidin-2-yl)-2-naphthyl]oxy]pentanoic acid (3)

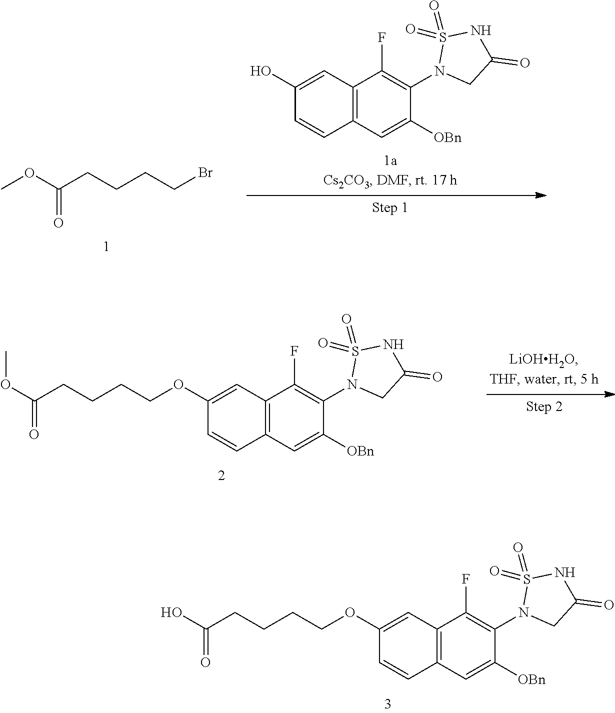

### Step 1: methyl 5-[[6-benzyloxy-8-fluoro-7-(1,1,4-trioxo-1,2,5-thiadiazolidin-2-yl)-2-naphthyl]oxy]pentanoate (2)

Into a 100 mL round bottom flask containing a well-stirred solution of 5-(3-benzyloxy-1-fluoro-7-hydroxy-2-naphthyl)-1,1-dioxo-1,2,5-thiadiazolidin-3-one (1, 0.3 g, 745.53 μmol) and methyl 5-bromopentanoate (1a, 174.50 mg, 894.64 μmol) in DMF (8 mL) was added cesium carbonate (485.82 mg, 1.49 mmol). After 17 h, the reaction mixture was filtered and concentrated under reduced pressure to obtain methyl 5-[[6-benzyloxy-8-fluoro-7-(1,1,4-trioxo-1,2,5-thiadiazolidin-2-yl)-2-naphthyl]oxy]pentanoate (2, 0.37 g, 642.74 μmol, 86% yield) as an off-white solid. The material was used in the next step without further purification. LCMS (ES+): m/z 517.2 [M+H]⁺

### Step 2: 5-[[6-benzyloxy-8-fluoro-7-(1,1,4-trioxo-1,2,5-thiadiazolidin-2-yl)-2-naphthyl]oxy]pentanoic acid (3)

Into a 25 mL single neck round bottom flask containing a well-stirred solution of methyl 5-[[6-benzyloxy-8-fluoro-7-(1,1,4-trioxo-1,2,5-thiadiazolidin-2-yl)-2-naphthyl]oxy]pentanoate (2, 557.23 mg, 967.98 μmol) in THF (7 mL) was added lithium hydroxide monohydrate (162.47 mg, 3.87 mmol) in water (3 mL). After 5 h, the reaction mixture was diluted with water (30 mL) and extracted with DCM (3×15 mL). The aqueous layer was acidified with aqueous 1.5 N HCl solution and extracted with ethyl acetate (3×30 mL). The combined organic layer was dried over Na₂SO₄ and concentrated under reduced pressure to obtain 5-[[6-benzyloxy-8-fluoro-7-(1,1,4-trioxo-1,2,5-thiadiazolidin-2-yl)-2-naphthyl]oxy]pentanoic acid (3, 0.3 g, 543.41 μmol, 56% yield) as a light brown solid. LCMS (ES−): m/z 501.2 [M−H]⁻

### 5-[1-fluoro-3-hydroxy-7-(4-piperidyl)-2-naphthyl]-1,1-dioxo-1,2,5-thiadiazolidin-3-one (6)

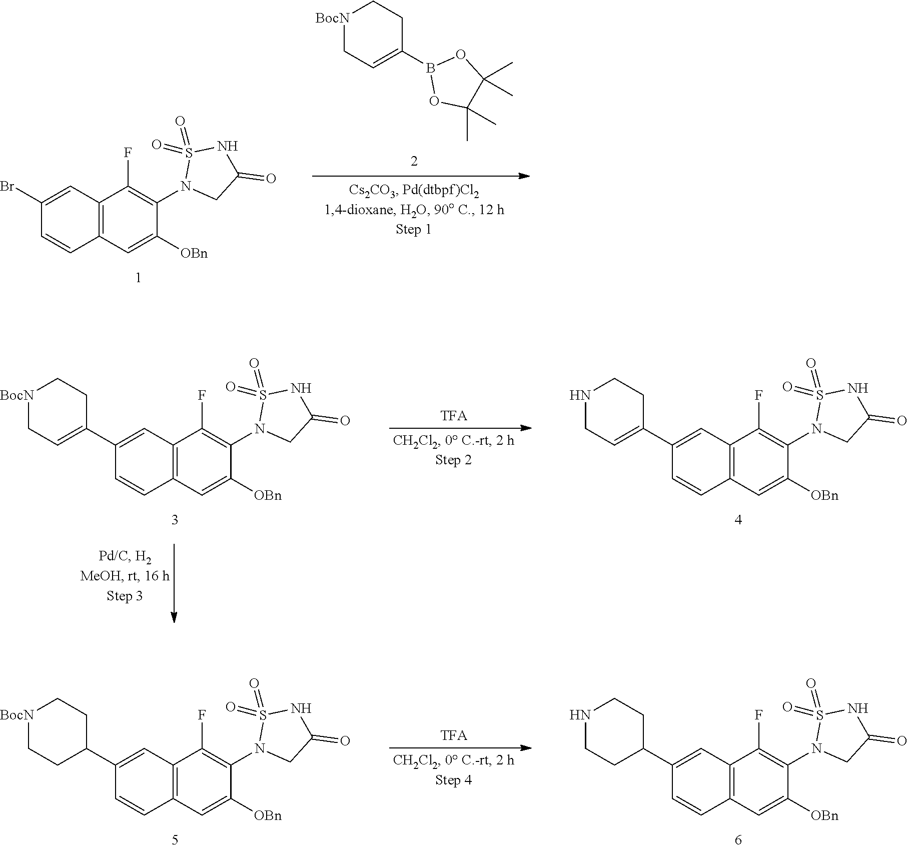 [SMILES: O=C1CN(c2c(OCc3ccccc3)cc3ccc(Br)cc3c2F)S(=O)(=O)N1 ; CC(C)(C)OC(=O)N1CC=C(B2OC(C)(C)C(C)(C)O2)CC1 ; CC(C)(C)OC(=O)N1CC=C(c2ccc3cc(OCc4ccccc4)c(N4CC(=O)NS4(=O)=O)c(F)c3c2)CC1 ; O=C1CN(c2c(OCc3ccccc3)cc3ccc(C4=CCNCC4)cc3c2F)S(=O)(=O)N1 ; CC(C)(C)OC(=O)N1CCC(c2ccc3cc(OCc4ccccc4)c(N4CC(=O)NS4(=O)=O)c(F)c3c2)CC1 ; O=C1CN(c2c(OCc3ccccc3)cc3ccc(C4CCNCC4)cc3c2F)S(=O)(=O)N1]

### Step 1: tert-butyl 4-[6-benzyloxy-8-fluoro-7-(1,1,4-trioxo-1,2,5-thiadiazolidin-2-yl)-2-naphthyl]-3,6-dihydro-2H-pyridine-1-carboxylate (3)

Into a 50 mL sealed tube containing a well-stirred solution of 5-(3-benzyloxy-7-bromo-1-fluoro-2-naphthyl)-1,1-dioxo-1,2,5-thiadiazolidin-3-one (1, 1 g, 2.15 mmol) and tert-butyl 4-(4,4,5,5-tetramethyl-1,3,2-dioxaborolan-2-yl)-3,6-dihydro-2H-pyridine-1-carboxy late (2, 731.00 mg, 2.36 mmol) in 1,4-dioxane (10 mL) and water (5 mL) was added Cs₂CO₃ (2.10 g, 6.45 mmol) and the reaction mixture was degassed by bubbling nitrogen through the solution for 5 min. Subsequently, PdCl₂(dtbpf) (112.06 mg, 171.93 μmol) was added to the reaction mixture and the resulting suspension was heated to 90° C. for 12 h. The reaction mixture was cooled to RT and poured into water (25 mL). The aqueous layer was extracted with EtOAc (3×30 mL). Organic layers were combined, dried over anhydrous Na₂SO₄, filtered and concentrated under reduced pressure. The residue was triturated with Et₂O (30 mL) to afford tert-butyl 4-[6-benzyloxy-8-fluoro-7-(1,1,4-trioxo-1,2,5-thiadiazolidin-2-yl)-2-naphthyl]-3,6-dihydro-2H-pyridine-1-carboxylate (3, 1.2 g, 1.88 mmol, 88% yield) as a brown solid. LCMS (ES+): m/z 468.1 [M-Boc+H]⁺

### Step 2: 5-[3-benzyloxy-1-fluoro-7-(1,2,3,6-tetrahydropyridin-4-yl)-2-naphthyl]-1,1-dioxo-1,2,5-thiadiazolidin-3-one (4)

Into a 50 mL single neck round bottom flask containing a well-stirred solution of tert-butyl 4-[6-benzyloxy-8-fluoro-7-(1,1,4-trioxo-1,2,5-thiadiazolidin-2-yl)-2-naphthyl]-3,6-dihydro-2H-pyridine-1-carboxy late (3, 600 mg, 940.76 μmol) in dry DCM (5 mL) was added TFA (536.34 mg, 4.70 mmol, 362.39 μL) under nitrogen atmosphere at 0° C. The reaction was stirred for 2 h at ambient temperature. The reaction mixture was concentrated under reduced pressure and the residue co-distilled with toluene (2×15 mL) and triturated with diethyl ether (20 mL) to afford 5-[3-benzyloxy-1-fluoro-7-(1,2,3,6-tetrahydropyridin-4-yl)-2-naphthyl]-1,1-dioxo-1,2,5-thiadiazolidin-3-one (4, 600 mg, 647.94 μmol, 69% yield, 63% purity, TFA salt) as an off-white solid. LCMS (ES+): m/z 468.0 [M+H]⁺

### Step 3: tert-butyl 4-[8-fluoro-6-hydroxy-7-(1,1,4-trioxo-1,2,5-thiadiazolidin-2-yl)-2-naphthyl]piperidine-1-carboxylate (5)

Into a 50 mL single neck, round bottom flask containing a well-stirred solution of tert-butyl 4-[6-benzyloxy-8-fluoro-7-(1,1,4-trioxo-1,2,5-thiadiazolidin-2-yl)-2-naphthyl]-3,6-dihydro-2H-pyridine-1-carboxylate (3, 350 mg, 548.78 μmol) in methanol (7 mL) was added Pd/C (10% wet) (311.50) mg, 2.93 mmol) under nitrogen atmosphere at ambient temperature. The reaction was hydrogenated for 16 h with a hydrogen bladder. The reaction mixture was filtered through a pad of Celite and washed with methanol (40 mL). Concentration under reduced pressure afforded tert-butyl 4-[8-fluoro-6-hydroxy-7-(1,1,4-trioxo-1,2,5-thiadiazolidin-2-yl)-2-naphthyl]piperidine-1-carboxylate (5, 300 mg, 493.37 μmol, 90% yield) as a pale-yellow solid. LCMS (ES+): m/z 380.1 [M-Boc+H]⁺

### Step 4: 5-[1-fluoro-3-hydroxy-7-(4-piperidyl)-2-naphthyl]-1,1-dioxo-1,2,5-thiadiazolidin-3-one (6)

Into a 50 mL single neck, round bottom flask containing a well-stirred solution of tert-butyl 4-[8-fluoro-6-hydroxy-7-(1,1,4-trioxo-1,2,5-thiadiazolidin-2-yl)-2-naphthyl]piperidine-1-carboxy late (5, 250) mg, 410.83 μmol) in dry DCM (5 mL) was added TFA (234.22 mg, 2.05 mmol, 158.26 μL) at (° C.) The reaction mixture was stirred for 2 h at RT and then concentrated under reduced pressure. The residue was triturated with diethyl ether (20 mL) to afford 5-[1-fluoro-3-hydroxy-7-(4-piperidyl)-2-naphthyl]-1,1-dioxo-1,2,5-thiadiazolidin-3-one (6, 200 mg, 360.74 μmol, 88% yield, TFA salt) as an off-white solid. LCMS (ES+): m/z 380.1 [M+H]⁺

### 1-[6-benzyloxy-8-fluoro-7-(1,1,4-trioxo-1,2,5-thiadiazolidin-2-yl)-2-naphthyl]pyrazole-4-carbaldehyde (3)

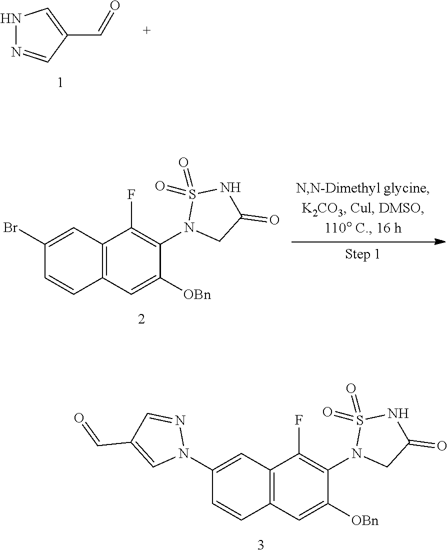 [SMILES: O=Cc1cn[nH]c1 ; O=C1CN(c2c(OCc3ccccc3)cc3ccc(Br)cc3c2F)S(=O)(=O)N1 ; O=Cc1cnn(-c2ccc3cc(OCc4ccccc4)c(N4CC(=O)NS4(=O)=O)c(F)c3c2)c1]

### Step 1: 1-[6-benzyloxy-8-fluoro-7-(1,1,4-trioxo-1,2,5-thiadiazolidin-2-yl)-2-naphthyl]pyrazole-4-carbaldehyde (3)

Into a 25 mL pressure tube containing a well-stirred solution of 5-(3-benzyloxy-7-bromo-1-fluoro-2-naphthyl)-1,1-dioxo-1,2,5-thiadiazolidin-3-one (2, 500 mg, 1.07 mmol) in DMSO (7 mL) were added 1H-pyrazole-4-carbaldehyde (1, 154.88 mg, 1.61 mmol) and N,N-dimethyl glycine (22.16 mg, 214.92 μmol) and potassium carbonate (207.93 mg, 1.50 mmol, 90.80 μL) and copper (I) iodide (61.40 mg, 322.38 μmol). The reaction mixture was heated at 110° C. After 16 h, the reaction mixture was filtered through Celite and washed with ethyl acetate (200 mL). The filtrate was concentrated to dryness and the residue was purified by reverse phase column chromatography [Purification method: Siliasep premium C18, 25 μm, 120 g column; Mobile phase A: 0.1% TFA in water; Mobile phase B: Acetonitrile] to afford 1-[6-benzyloxy-8-fluoro-7-(1,1,4-trioxo-1,2,5-thiadiazolidin-2-yl)-2-naphthyl]pyrazole-4-carbaldehyde (3, 150 mg, 298.08 μmol, 69% yield) as a pale yellow solid. LCMS (ES+): m/z 480.0 [M+H]⁺

### 4-[[6-benzyloxy-8-fluoro-7-(1,1,4-trioxo-1,2,5-thiadiazolidin-2-yl)-2-naphthyl]oxy]butanal (4)

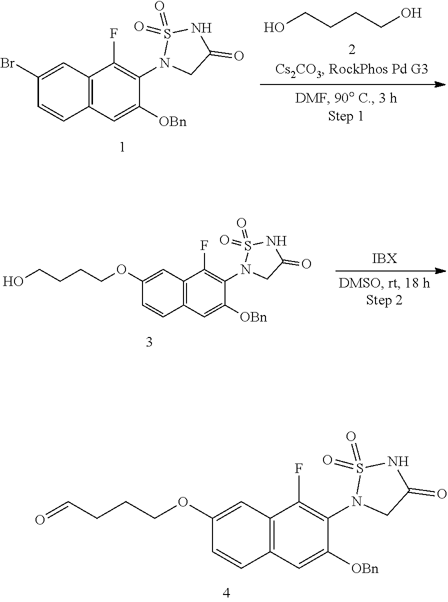 [SMILES: O=C1CN(c2c(OCc3ccccc3)cc3ccc(Br)cc3c2F)S(=O)(=O)N1 ; OCCCCO ; O=C1CN(c2c(OCc3ccccc3)cc3ccc(OCCCCO)cc3c2F)S(=O)(=O)N1 ; O=CCCCOc1ccc2cc(OCc3ccccc3)c(N3CC(=O)NS3(=O)=O)c(F)c2c1]

### Step 1: 5-[3-benzyloxy-1-fluoro-7-(4-hydroxybutoxy)-2-naphthyl]-1,1-dioxo-1,2,5-thiadiazolidin-3-one (3)

A 100 mL sealed tube was charged with 5-(3-benzyloxy-7-bromo-1-fluoro-2-naphthyl)-1,1-dioxo-1,2,5-thiadiazolidin-3-one (1, 1.0 g, 2.15 mmol), butane-1,4-diol (2, 968.43 mg, 10.75 mmol), DMF (5 mL) and Cs₂CO₃ (2.10 g, 6.45 mmol). The resulting suspension was purged with nitrogen for 10 min. RockPhos Pd G3 (90.10 mg, 107.46 μmol) was added and the reaction mixture was stirred at 90° C. for 3 h. The reaction mixture was filtered through a pad of Celite and washed thoroughly with dioxane (50 mL) and THF (50 mL). The filtrate was concentrated under reduced pressure and purified by reverse-phase preparative HPLC [Column: SILIASEP C18, 25 μm, 120 g; 0.1% TFA in water: acetonitrile] to afford 5-[3-benzyloxy-1-fluoro-7-(4-hydroxy butoxy)-2-naphthyl]-1,1-dioxo-1,2,5-thiadiazolidin-3-one (3, 350 mg, 560.22 μmol, 26% yield, TFA salt) as an off-white solid. LCMS (ES+): m/z 475.2 [M+H]⁺

### Step 2: 4-[[6-benzyloxy-8-fluoro-7-(1,1,4-trioxo-1,2,5-thiadiazolidin-2-yl)-2-naphthyl]oxy]butanal (4)

Into a 50 mL single neck round bottom flask containing a well-stirred solution of 5-[3-benzyloxy-1-fluoro-7-(4-hydroxy butoxy)-2-naphthyl]-1,1-dioxo-1,2,5-thiadiazolidin-3-one (3, 330 mg, 695.47 μmol) in DMSO (3 mL) was added 2-iodoxybenzoic acid (311.59 mg, 1.11 mmol) and stirred at RT for 18 h. The reaction mixture was concentrated under reduced pressure and directly taken for next step. LCMS (ES−): m/z 471.0 [M−H]⁻

### 2-[4-[6-benzyloxy-8-fluoro-7-(1,1,4-trioxo-1,2,5-thiadiazolidin-2-yl)-2-naphthyl]pyrazol-1-yl]acetaldehyde (6)

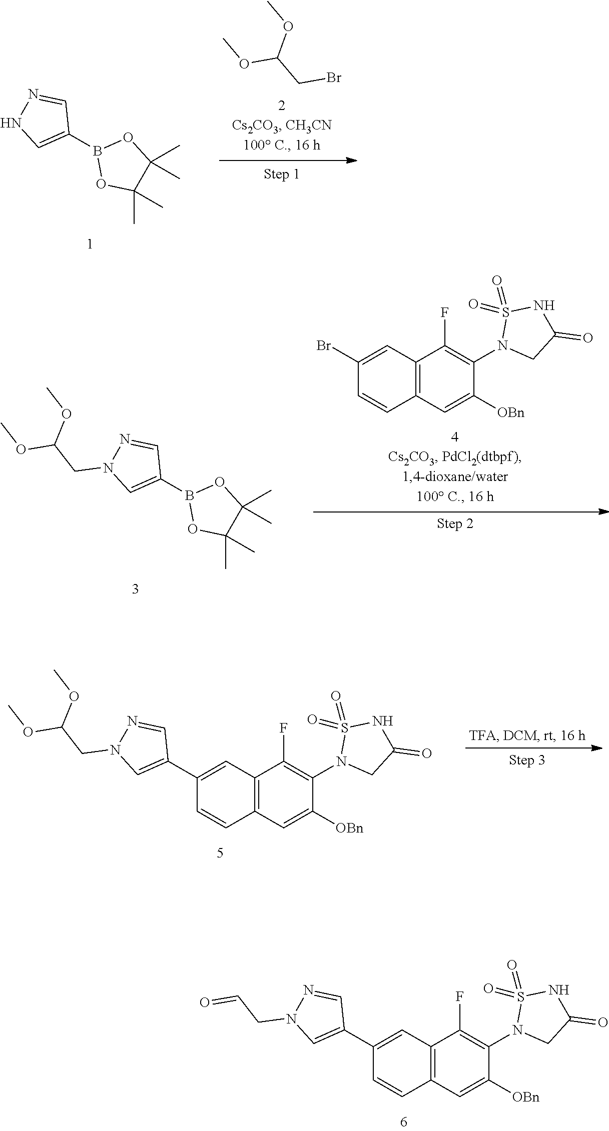

### Step 1: 1-(2,2-dimethoxyethyl)-4-(4,4,5,5-tetramethyl-1,3,2-dioxaborolan-2-yl)pyrazole (3)

Into a 100 mL pressure tube containing a well-stirred solution of 4-(4,4,5,5-tetramethyl-1,3,2-dioxaborolan-2-yl)-1H-pyrazole (1, 1.11 g, 5.15 mmol) in acetonitrile (40 mL) were added cesium carbonate (4.20 g, 12.88 mmol) and 2-bromo-1,1-dimethoxy-ethane (2, 967.83 mg, 5.15 mmol, 676.81 uL). The reaction mixture was heated to 100° C. for. After 16 h, the reaction mixture was diluted with water (100 mL) and extracted with EtOAc (3×300 mL). The combined organic layer was washed with brine solution (150 mL), dried over Na₂SO₄, filtered and concentrated to afford 1-(2,2-dimethoxyethyl)-4-(4,4,5,5-tetramethyl-1,3,2-dioxaborolan-2-yl)pyrazole (3, 850 mg, 1.84 mmol, 36% yield) as a pale yellow liquid. The material was used in the next step without purification. LCMS (ES+): m/z 283.2 [M+H]⁺

### Step 2: 5-[3-benzyloxy-7-[1-(2,2-dimethoxyethyl)pyrazol-4-yl]-1-fluoro-2-naphthyl]-1,1-dioxo-1,2,5-thiadiazolidin-3-one (5)

In a 25 mL pressure tube containing a well-stirred solution of 1-(2,2-dimethoxyethyl)-4-(4,4,5,5-tetramethyl-1,3,2-dioxaborolan-2-yl)pyrazole (3, 491.08 mg, 1.06 mmol) and 5-(3-benzyloxy-7-bromo-1-fluoro-2-naphthyl)-1,1-dioxo-1,2,5-thiadiazolidin-3-one (4, 549.71 mg, 1.06 mmol) in 1,4-dioxane (8 mL) and water (2 mL) was added cesium carbonate (1.04 g, 3.19 mmol). The suspension was degassed by bubbling nitrogen gas for 10 min. Then, [1,1′-Bis(di-tert-butylphosphino)ferrocene]dichloropalladium(II) (69.30 mg, 106.33 μmol) was added. The reaction mixture was heated at 0° C. After 16 h, the mixture was filtered through Celite and washed with ethyl acetate (100 mL). The filtrate was concentrated to dryness and the residue was triturated with diethyl ether (2×10 mL), filtered and dried to obtain 5-[3-benzyloxy-7-[1-(2,2-dimethoxyethyl)pyrazol-4-yl]-1-fluoro-2-naphthyl]-1,1-dioxo-1,2,5-thiadiazolidin-3-one (5, 300 mg, 523.34 μmol, 49% yield) as a pale yellow solid. LCMS (ES+): m/z 541.0 [M+H]⁺

### Step 3: 2-[4-[6-benzyloxy-8-fluoro-7-(1,1,4-trioxo-1,2,5-thiadiazolidin-2-yl)-2-naphthyl]pyrazol-1-yl]acetaldehyde (6)

In a 50 mL single neck round bottom flask containing a well-stirred solution of 5-[3-benzyloxy-7-[1-(2,2-dimethoxyethyl)pyrazol-4-yl]-1-fluoro-2-naphthyl]-1,1-dioxo-1,2,5-thiadiazolidin-3-one (5, 300) mg, 510.58 μmol) in DCM (2.5 mL) was added TFA (4.08 g, 35.82 mmol, 2.76 mL) at (° C.) After 16 h, the volatiles were removed under reduced pressure and the residue was triturated with diethyl ether (10 mL) and dried to afford 2-[4-[6-benzyloxy-8-fluoro-7-(1,1,4-trioxo-1,2,5-thiadiazolidin-2-yl)-2-naphthyl]pyrazol-1-yl]acetaldehyde (6, 400 mg, 364.82 μmol, 71% yield, 56% purity) as a brown solid. LCMS (ES+): m/z 495.0 [M+H]⁺

### 2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]acetic acid (4)

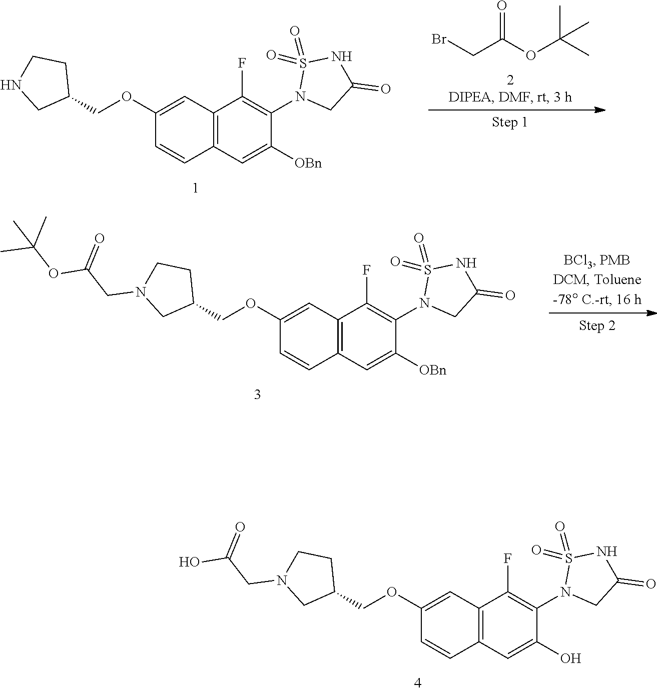

### Step 1: tert-butyl 2-[(3S)-3-[[6-benzyloxy-8-fluoro-7-(1,1,4-trioxo-1,2,5-thiadiazolidin-2-yl)-2-naphthyl]oxymethyl]pyrrolidin-1-yl]acetate (3)

Into a 50 mL single neck round bottom flask containing a well-stirred solution of 5-[3-benzyloxy-1-fluoro-7-[[(3S)-pyrrolidin-3-yl]methoxy]-2-naphthyl]-1,1-dioxo-1,2,5-thiadiazolidin-3-one (1, 300 mg, 574.73 μmol, HCl salt) in anhydrous DMF (5 mL) were added DIPEA (239.77 mg, 1.86 mmol, 323.14 μL) and tert-butyl-2-bromoacetate (2, 144.75 mg, 742.10 μmol, 108.83 μL). After 3 h, the reaction mixture was diluted with water (25 mL) and extracted with EtOAc (50 mL×2). The combined organic layer was washed with brine solution (20 mL) and dried over anhydrous Na₂SO₄. The solvent was removed under reduced pressure and the residue was purified by reverse phase column chromatography [Purification method: Silycycle-C18 (150×19) mm 5 micron column; Mobile phase A: 0.1% TFA in water; Mobile phase B: Acetonitrile] to obtain tert-butyl 2-[(35)-3-[[6-benzyloxy-8-fluoro-7-(1,1,4-trioxo-1,2,5-thiadiazolidin-2-yl)-2-naphthyl]oxymethyl]pyrrolidin-1-yl]acetate (3, 280 mg, 68% yield) as an off-white solid. LCMS (ES+): m/z 600 [M+H]⁺

### Step 2: 2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]acetic acid (4)

Into a 25 mL single neck round bottom flask containing a well-stirred solution of tert-butyl 2-[(3S)-3-[[6-benzyloxy-8-fluoro-7-(1,1,4-trioxo-1,2,5-thiadiazolidin-2-yl)-2-naphthyl]oxymethyl]pyrrolidin-1-yl]acetate 3 (270 mg, 436.74 μmol) in a mixture of anhydrous toluene (2 mL) and DCM (2 mL) was added pentamethyl benzene (388.47 mg, 2.62 mmol) under nitrogen atmosphere. The reaction mixture was cooled to −78° C. Then boron trichloride (1.0 M in DCM) (2.61 mL, 2.61 mmol) was added dropwise. The reaction mixture was stirred at RT. After 16 h, the reaction mixture was cooled to −78° C. and quenched with 10% MeOH in DCM (10 mL). The volatiles were removed and the residue was triturated with diethyl ether (25 mL) and purified by reverse phase preparative HPLC [Method: Silycycle C18 (150×19) mm 5 micron column; Mobile phase A: 0.1% TFA in water; Mobile phase B: Acetonitrile] to afford 2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]acetic acid (4, 60 mg, 22% yield, TFA salt) as a brown solid. LCMS (ES+): m/z 454 [M+H]⁺

### 2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetic acid (4)

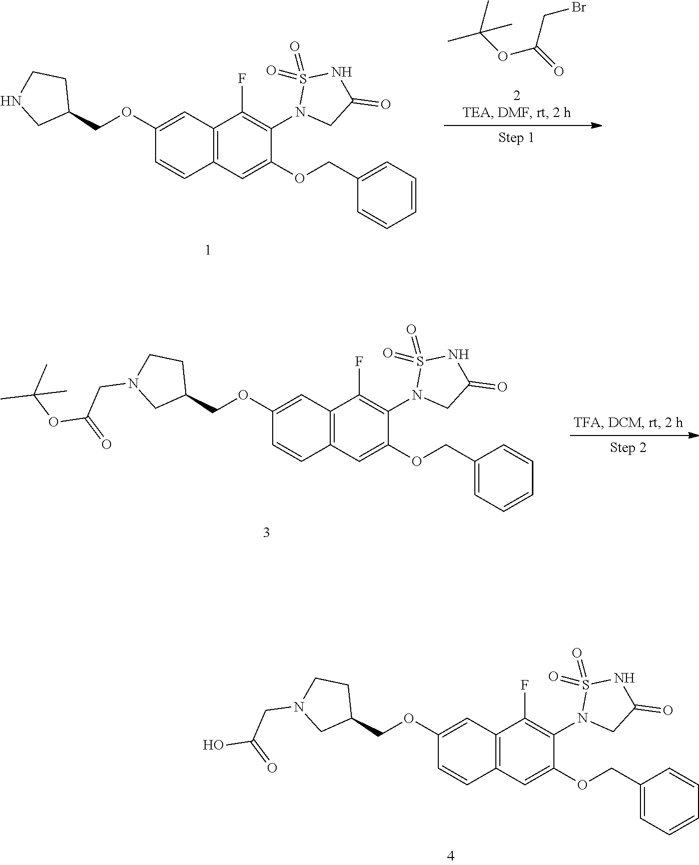

### Step 1: tert-butyl 2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetate (3)

Into a 25 mL single neck round bottom flask containing a well-stirred solution of 5-[3-benzyloxy-1-fluoro-7-[[(3R)-pyrrolidin-3-yl]methoxy]-2-naphthyl]-1,1-dioxo-1,2,5-thiadiazolidin-3-one (1, 300 mg, 574.73 μmol, HCl salt) in anhydrous DMF (6 mL) was added TEA (290.78 mg, 2.87 mmol, 400.53 μL) and tert-butyl 2-bromoacetate (2, 168.15 mg, 862.09 μmol, 126.43 μL) dropwise at 0° C. After 2 h at RT, the reaction mixture was quenched with water (40 mL) and extracted with EtOAc (3×30 mL). The combined organic layer was washed with cold water (2×30 mL), brine solution (30 mL), dried over anhydrous sodium sulfate, filtered and concentrated under reduced pressure to afford tert-butyl 2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetate (3, 180 mg, 299.27 μmol, 52% yield) as an off-white solid. LCMS (ES+): m/z 600.0 [M+H]⁺

### Step 2: 2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetic acid (4)

Into a 50 mL single neck round bottom flask containing a well-stirred solution of tert-butyl 2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetate (3, 175 mg, 291.13 μmol) in DCM (4 mL) was added TFA (1.48 g, 12.98 mmol, 1 mL) at (° C.) After 2 h at RT, the volatiles were removed under reduced pressure and the residue was triturated with Et₂O (20 mL), filtered and dried to afford 2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetic acid (4, 170 mg, 241.98 μmol, 83% yield. TFA salt) as an off-white solid. LCMS (ES+): m/z 544.0 [M+H]⁺

### tert-butyl (R)-3-(7-(1,1-dioxido-4-oxo-1,2,5-thiadiazolidin-2-yl)-8-fluoro-6-hydroxynaphthalen-2-yl)pyrrolidine-1-carboxylate (2a, first eluted fraction) and tert-butyl (S)-3-(7-(1,1-dioxido-4-oxo-1,2,5-thiadiazolidin-2-yl)-8-fluoro-6-hydroxynaphthalen-2-yl)pyrrolidine-1-carboxylate (2b, Second Eluted Fraction)

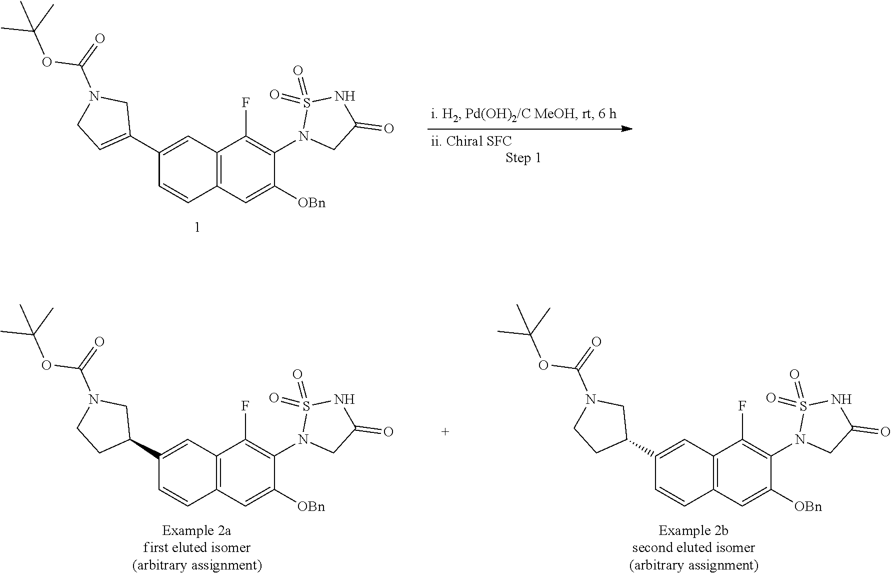 [SMILES: CC(C)(C)OC(=O)N1CC=C(c2ccc3cc(OCc4ccccc4)c(N4CC(=O)NS4(=O)=O)c(F)c3c2)C1 ; CC(C)(C)OC(=O)N1CC[C@H](c2ccc3cc(OCc4ccccc4)c(N4CC(=O)NS4(=O)=O)c(F)c3c2)C1 ; CC(C)(C)OC(=O)N1CC[C@@H](c2ccc3cc(OCc4ccccc4)c(N4CC(=O)NS4(=O)=O)c(F)c3c2)C1]

### Step 1: tert-butyl (R)-3-(7-(1,1-dioxido-4-oxo-1,2,5-thiadiazolidin-2-yl)-8-fluoro-6-hydroxynaphthalen-2-yl)pyrrolidine-1-carboxylate (2a, first eluted fraction) and tert-butyl (S)-3-(7-(1,1-dioxido-4-oxo-1,2,5-thiadiazolidin-2-yl)-8-fluoro-6-hydroxynaphthalen-2-yl)pyrrolidine-1-carboxylate (2b, second eluted fraction)

Into a 50 mL single neck round bottom flask containing a well-stirred solution of tert-butyl 3-[6-benzyloxy-8-fluoro-7-(1,1,4-trioxo-1,2,5-thiadiazolidin-2-yl)-2-naphthyl]-2,5-dihydropyrrole-1-carboxylate (1, 1.4 g, 2.53 mmol) in methanol (20) mL) was added Palladium hydroxide on carbon (20) wt. % 50% water) (1.07 g, 7.59 mmol) under nitrogen atmosphere at ambient temperature. The reaction mixture was hydrogenated under hydrogen bladder pressure for 6 h. The reaction mixture was filtered through Celite and washed with methanol (200 mL). The filtrate was concentrated under reduced pressure and triturated with diethyl ether to afford tert-butyl 3-(7-(1,1-dioxido-4-oxo-1,2,5-thiadiazolidin-2-yl)-8-fluoro-6-hydroxynaphthalen-2-yl)pyrrolidine-1-carboxylate (2a/b, 1.05 g) as an off-white solid. The enantiomers were separated by chiral SFC: Method details: Column Name: Chiralcel OZ-H: Co-Solvent: 40% and Co-Solvent Name: 0.5% Isopropyl Amine in MeOH: Outlet Pressure: 100 bar; Temperature: 35° C. After concentration, the first eluted fraction at RT 3.83 min: tert-butyl (R)-3-(7-(1,1-dioxido-4-oxo-1,2,5-thiadiazolidin-2-yl)-8-fluoro-6-hydroxynaphthalen-2-yl)pyrrolidine-1-carboxylate (2a, first eluted fraction, 350 mg, 714.75 μmol, 28% yield) was isolated as an off-white solid. LCMS (ES+): m/z 410.0 [M-tBu+H]⁺

¹H NMR (400 MHZ, DMSO-d₆): δ 10.06 (brs, 1H), 7.76-7.72 (m, 2H), 7.47 (d, J=10.00 Hz, 1H), 7.07 (s, 1H), 4.24 (s, 2H), 3.76-3.71 (m, 1H), 3.55-3.34 (m, 2H), 3.35-3.23 (m, 2H), 2.34-2.25 (m, 1H), 2.08-2.03 (m, 1H), 1.43 (s, 9H).

Second eluted fraction at RT 5.28 min: tert-butyl (S)-3-(7-(1,1-dioxido-4-oxo-1,2,5-thiadiazolidin-2-yl)-8-fluoro-6-hydroxynaphthalen-2-yl)pyrrolidine-1-carboxylate (2b, second eluted fraction, 255 mg, 523.26 μmol, 21% yield) was isolated as an off-white solid. LCMS (ES+): m/z 409.9 [M-tBu+H]⁺

### tert-butyl N-[[7-benzyloxy-5-fluoro-6-(1,1,4-trioxo-1,2,5-thiadiazolidin-2-yl)naphthalene-2-carbonyl]amino]carbamate (5)

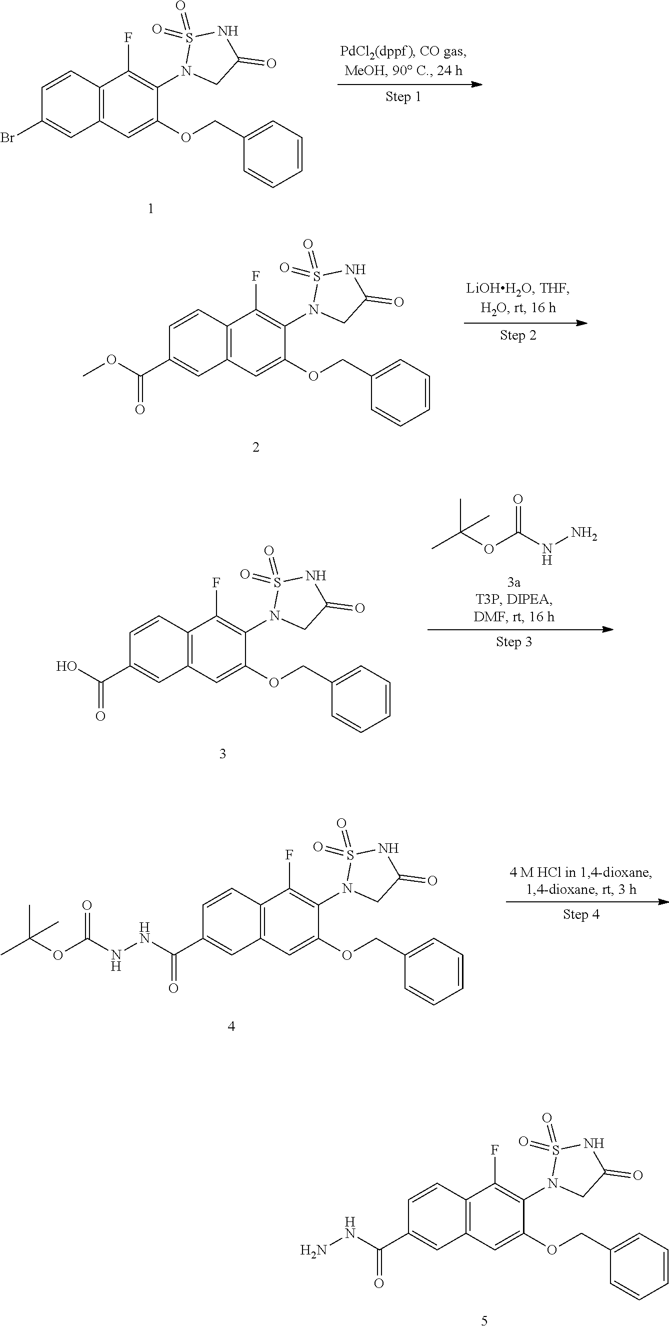

### Step 1: methyl 7-benzyloxy-5-fluoro-6-(1,1,4-trioxo-1,2,5-thiadiazolidin-2-yl)naphthalene-2-carboxylate (2)

To a stirred solution of 5-(3-benzyloxy-6-bromo-1-fluoro-2-naphthyl)-1,1-dioxo-1,2,5-thiadiazolidin-3-one (1, 2.5 g, 5.37 mmol) in methanol (15 mL) was added triethylamine (2.72 g, 26.86 mmol, 3.74 mL) and the solution was purged with nitrogen for 10 min. Then, [1,1′-bis(diphenylphosphino)ferrocene]dichloropalladium(II) (196.43 mg, 268.65 μmol) was added. The reaction mixture was heated at 90° C. under carbon monoxide atmosphere (5 kg pressure). After 24 h, the reaction mixture was filtered through Celite and washed with methanol (30 mL). The filtrate was concentrated under reduced pressure to obtain methyl 7-benzyloxy-5-fluoro-6-(1,1,4-trioxo-1,2,5-thiadiazolidin-2-yl)naphthalene-2-carboxylate (2, 3.8 g, 5.33 mmol, 99% yield, 62% purity) as a brown solid. The material was used in the next step without purification. LCMS (ES−): m/z 443.0 [M−H]⁻

### Step 2: 7-benzyloxy-5-fluoro-6-(1,1,4-trioxo-1,2,5-thiadiazolidin-2-yl)naphthalene-2-carboxylic acid (3)

Into a 100 mL single neck round bottom flask containing a well-stirred solution of methyl 7-benzyloxy-5-fluoro-6-(1,1,4-trioxo-1,2,5-thiadiazolidin-2-yl)naphthalene-2-carboxylate (2, 3.8 g, 5.30 mmol) in THF (5 mL) was added a solution of lithium hydroxide monohydrate (1.11 g, 26.51 mmol) in water (5 mL). After 16 h, the reaction mixture was acidified with aqueous 1.5 N HCl solution. The mixture was extracted with ethyl acetate (3×70 mL). The combined organic layer was washed with water (100 mL), brine (100 mL) and dried over sodium sulfate. The solvent was removed under reduced pressure and the residue purified by reverse phase column chromatography [Purification method: Biotage, C-18, 120 g column; Mobile phase A: 0.1% formic acid in water; Mobile phase B: Acetonitrile] to afford 7-benzyloxy-5-fluoro-6-(1,1,4-trioxo-1,2,5-thiadiazolidin-2-yl)naphthalene-2-carboxylic acid (3, 1.25 g, 2.89 mmol, 54% yield) as a colorless solid.

¹H NMR (400 MHZ, DMSO-d₆): δ 13.00 (bs, 1H), 8.56 (s, 1H), 8.09 (d, J=8.40 Hz, 1H), 7.95 (dd, J=1.60, 8.60 Hz, 1H), 7.71 (s, 1H), 7.56-7.54 (m, 2H), 7.42-7.35 (m, 3H), 5.31 (s, 2H), 4.51 (s, 2H).

### Step 3: tert-butyl N-[[7-benzyloxy-5-fluoro-6-(1,1,4-trioxo-1,2,5-thiadiazolidin-2-yl)naphthalene-2-carbonyl]amino]carbamate (4)

Into a 100 mL round bottom flask containing a well-stirred solution of 7-benzyloxy-5-fluoro-6-(1,1,4-trioxo-1,2,5-thiadiazolidin-2-yl)naphthalene-2-carboxylic acid (3, 1.03 g, 2.35 mmol) and tert-butyl N-aminocarbamate (3a, 621.10 mg, 4.70 mmol) in DMF (5 mL) was added DIPEA (911.06 mg, 7.05 mmol, 1.23 mL) followed by propylphosphonic anhydride (50% w/v solution in ethyl acetate) (2.24 g, 3.52 mmol, 50% purity). After 16 h, the reaction mixture was concentrated under reduced pressure and purified by reverse phase column chromatography [Purification method: Biotage, 120 g C-18 column; Mobile phase A: 0.1% TFA in water; Mobile phase B: Acetonitrile] to obtain tert-butyl N-[[7-benzyloxy-5-fluoro-6-(1,1,4-trioxo-1,2,5-thiadiazolidin-2-yl)naphthalene-2-carbonyl]amino]carbamate (4, 1.2 g, 1.90 mmol, 81% yield) as a light yellow solid. LCMS (ES−): m/z 543.0 [M−H]⁻

### Step 4: tert-butyl N-[[7-benzyloxy-5-fluoro-6-(1,1,4-trioxo-1,2,5-thiadiazolidin-2-yl)naphthalene-2-carbonyl]amino]carbamate (5)

Into a 25 mL round bottom flask containing a well-stirred solution of tert-butyl 2-(7-(benzyloxy)-6-(1,1-dioxido-4-oxo-1,2,5-thiadiazolidin-2-yl)-5-fluoro-2-naphthoyl)hydrazine-1-carboxylate (4, 1.2 g, 1.90 mmol) in 1,4-dioxane (5 mL) was added HCl (4M in 1,4-dioxane) (2.0 g, 55.09 mmol, 13.5 mL) dropwise at 0° C. After 3 h at RT, the volatiles were removed under reduced pressure. The residue was triturated with diethyl ether (2×25 mL), filtered and dried to afford 7-benzyloxy-5-fluoro-6-(1,1,4-trioxo-1,2,5-thiadiazolidin-2-yl)naphthalene-2-carbohydrazide (5, 0.9 g, 1.70 mmol, 90% yield, HCl salt) as an off-white solid. LCMS (ES+): m/z 445.0 [M+H]⁺

### 4-[[6-benzyloxy-8-fluoro-7-(1,1,4-trioxo-1,2,5-thiadiazolidin-2-yl)-2-naphthyl]oxy]-2,2-dimethyl-butanal (6)

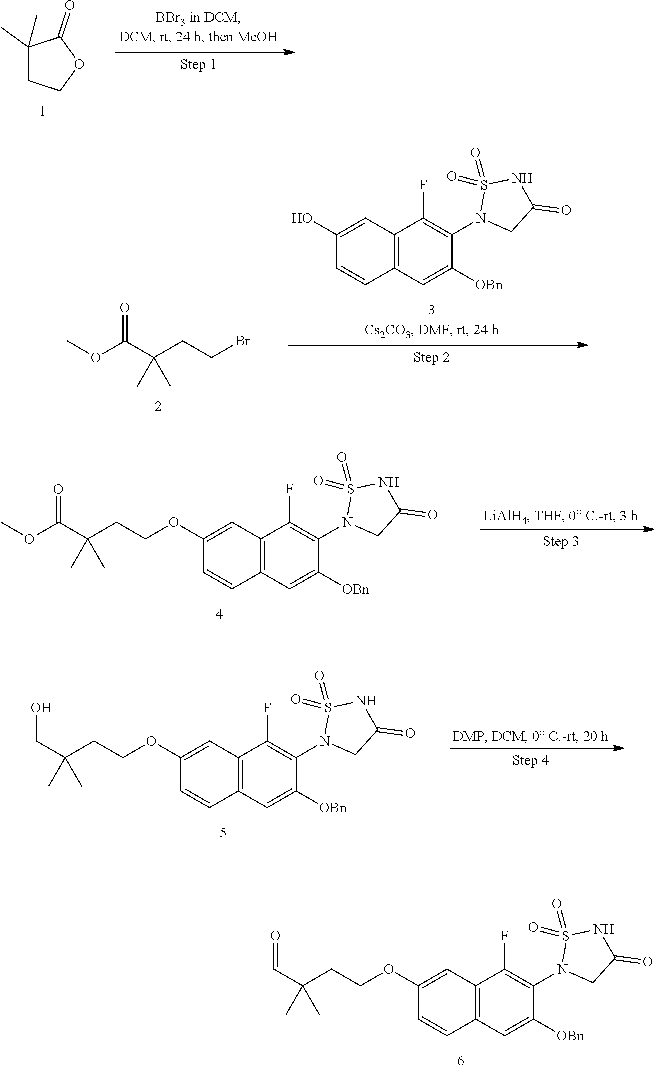

### Step 1: methyl 4-bromo-2,2-dimethyl-butanoate (2)

To a 250 mL three neck round bottom flask containing a solution of 3,3-dimethyltetrahydrofuran-2-one (1, 3 g, 26.28 mmol) in DCM (2 mL) was added boron tribromide (1M in DCM) (9.88 g, 39.42 mmol, 39.5 mL) at 0° C. After 24 h at RT, the reaction mixture was cooled to 0° C. and quenched with drop-wise addition of methanol (8 mL). The mixture was then stirred at ambient temperature for 1 h and diluted with DCM (70 mL). The solution was washed with aqueous saturated NaHCO₃ solution (50 mL). The aqueous layer was back-extracted with DCM (3×40 mL). The combined organic layer was washed with brine solution (75 mL), dried over sodium sulfate, filtered and solvent removed. The residue was purified by flash silica gel column chromatography (3-5% EtOAc in petroleum ether) to obtain methyl 4-bromo-2,2-dimethyl-butanoate (2, 1.41 g, 6.07 mmol, 23% yield) as a yellow liquid.

¹H NMR (400 MHZ, CDCl₃): δ 3.71 (s, 3H), 3.38-3.34 (m, 2H), 2.19-2.15 (m, 2H), 1.24 (s, 6H).

### Step 2: methyl 4-[[6-benzyloxy-8-fluoro-7-(1,1,4-trioxo-1,2,5-thiadiazolidin-2-yl)-2-naphthyl]oxy]-2,2-dimethyl-butanoate (4)

To a 50 mL single neck round bottom flask containing a solution of 5-(3-benzyloxy-1-fluoro-7-hydroxy-2-naphthyl)-1,1-dioxo-1,2,5-thiadiazolidin-3-one (3, 1.2 g, 2.98 mmol) in DMF (6 mL), was added cesium carbonate (2.91 g, 8.95 mmol), followed by methyl 4-bromo-2,2-dimethyl-butanoate (2, 762.06 mg, 3.28 mmol) in DMF (2 mL). After 24 h, additional cesium carbonate (0.145 g, 0.447 mmol) and methyl 4-bromo-2,2-dimethyl-butanoate (2, 38 mg, 0.164 mmol) were added and continued stirring for 4 h. The reaction mixture was filtered and washed with DMF (2×5 mL). The filtrate was concentrated under reduced pressure and purified by reverse phase column chromatography [Purification method: Siliasep C18 120 g column; Mobile phase A: 0.1% TFA in water; Mobile phase B: MeCN] to afford methyl 4-[[6-benzyloxy-8-fluoro-7-(1,1,4-trioxo-1,2,5-thiadiazolidin-2-yl)-2-naphthyl]oxy]-2,2-dimethyl-butanoate (4, 1.35 g, 2.52 mmol, 84% yield) as a pale grey solid. LCMS (ES+): m/z 531 [M+H]⁺

### Step 3: 5-[3-benzyloxy-1-fluoro-7-(4-hydroxy-3,3-dimethyl-butoxy)-2-naphthyl]-1,1-dioxo-1,2,5-thiadiazolidin-3-one (5)

To a 100 mL two neck round bottom flask containing a solution of methyl 4-[[6-benzyloxy-8-fluoro-7-(1,1,4-trioxo-1,2,5-thiadiazolidin-2-yl)-2-naphthyl]oxy]-2,2-dimethyl-butanoate (4, 1.35 g, 2.52 mmol) in THF (30 mL) under nitrogen atmosphere, was added a solution of lithium aluminum hydride (1.0 M in THF) (105.17 mg, 2.77 mmol, 2.8 mL) at (° C.) The reaction mixture was stirred at ambient temperature for 3 h. The reaction mixture was quenched with ice-water (10) mL) and concentrated under reduced pressure. The material was purified by reverse phase column chromatography [Purification method: Siliasep C18 120 g column; Mobile phase A: 0.1% TFA in water; Mobile phase B: MeCN] to afford 5-[3-benzyloxy-1-fluoro-7-(4-hydroxy-3,3-dimethyl-butoxy)-2-naphthyl]-1,1-dioxo-1,2,5-thiadiazolidin-3-one (5, 580 mg, 934.82 μmol, 37% yield) as a pale brown solid. LCMS (ES+): m/z 503 [M+H]⁺

### Step 4: 4-[[6-benzyloxy-8-fluoro-7-(1,1,4-trioxo-1,2,5-thiadiazolidin-2-yl)-2-naphthyl]oxy]-2,2-dimethyl-butanal (6)

To a 50 mL single neck round bottom flask containing a solution of 5-[3-benzyloxy-1-fluoro-7-(4-hydroxy-3,3-dimethyl-butoxy)-2-naphthyl]-1,1-dioxo-1,2,5-thiadiazolidin-3-one (5, 580 mg, 934.82 μmol) in DCM (20 mL) was added Dess-Martin Periodinane (594.75 mg, 1.40 mmol) at (° C.) and the reaction mixture was stirred at ambient temperature for 20 h. The reaction mixture was diluted with DCM (50) mL) and washed with 10% aqueous NaHCO₃ solution (30) mL). The aqueous layer was back-extracted with DCM (3×50 mL). The combined DCM layer was washed with water (100 mL), brine solution (100 mL), dried over sodium sulfate, filtered and solvent removed to afford 4-[[6-benzyloxy-8-fluoro-7-(1,1,4-trioxo-1,2,5-thiadiazolidin-2-yl)-2-naphthyl]oxy]-2,2-dimethyl-butanal (6, 540 mg, 722.82 μmol, 77% yield, 67% purity) as a black solid, which was used without further purification in the next step. LCMS (ES+): m/z 501 [M+H]⁺

### 2-[4-[7-benzyloxy-5-fluoro-6-(1,1,4-trioxo-1,2,5-thiadiazolidin-2-yl)-2-naphthyl]pyrazol-1-yl]acetaldehyde (6)

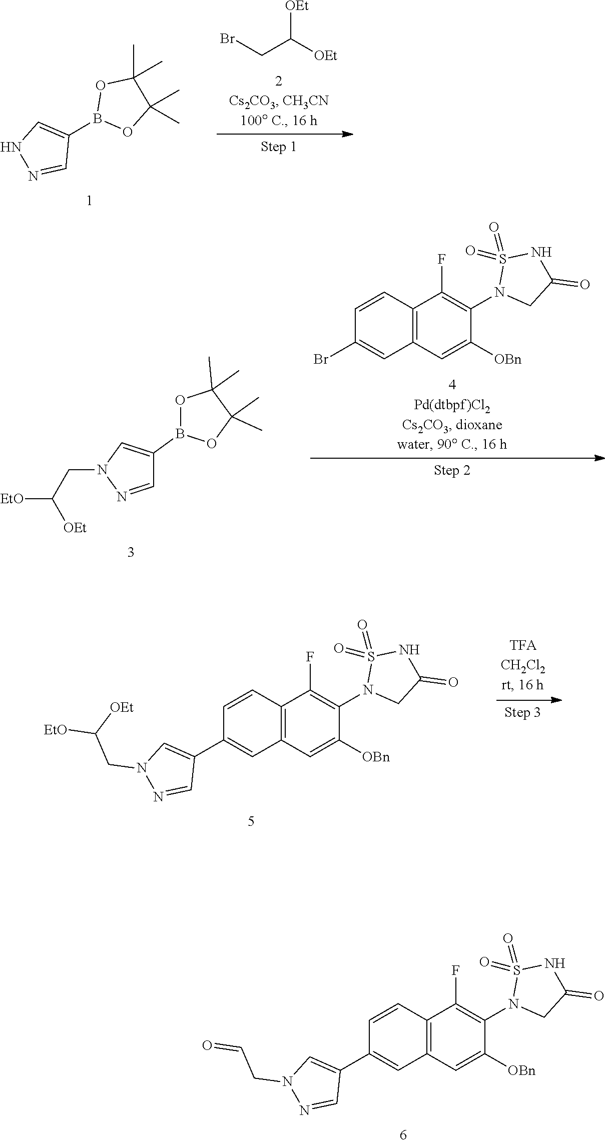 [SMILES: CC1(C)OB(c2cn[nH]c2)OC1(C)C ; CCOC(CBr)OCC ; CCOC(Cn1cc(B2OC(C)(C)C(C)(C)O2)cn1)OCC ; O=C1CN(c2c(OCc3ccccc3)cc3cc(Br)ccc3c2F)S(=O)(=O)N1 ; CCOC(Cn1cc(-c2ccc3c(F)c(N4CC(=O)NS4(=O)=O)c(OCc4ccccc4)cc3c2)cn1)OCC ; O=CCn1cc(-c2ccc3c(F)c(N4CC(=O)NS4(=O)=O)c(OCc4ccccc4)cc3c2)cn1]

### Step 1: 1-(2,2-diethoxyethyl)-4-(4,4,5,5-tetramethyl-1,3,2-dioxaborolan-2-yl)-1H-pyrazole (3)

In to a 250 mL sealed tube containing a well-stirred solution of 4-(4,4,5,5-tetramethyl-1,3,2-dioxaborolan-2-yl)-1H-pyrazole (1, 1 g, 5.15 mmol) in acetonitrile (20 mL) were added 2-bromo 1,1 diethoxy ethane (2, 0.39 mL, 2.58 mmol) and cesium carbonate (3.36 mL, 10.31 mmol). The reaction mixture was stirred at 100° C. After 16 h, the mixture was diluted with water (50 mL) and extracted with EtOAc (50 mL×2). The combined organic layers were washed with 1.5N HCl (20 mL), brine solution (25 mL) and dried over anhydrous Na₂SO₄ and filtered. The solvent was evaporated and the residue was purified by reverse phase prep HPLC [Purification method: X-Select C18 column; Mobile phase A: 0.1% Formic acid in water; Mobile phase B: acetonitrile) to obtain 1-(2,2-diethoxyethyl)-4-(4,4,5,5-tetramethyl-1,3,2-dioxaborolan-2-yl)-1H-pyrazole 3 (550 mg, 25% yield, formic acid salt) as a colorless gummy mass.

### Step 2: 5-[3-benzyloxy-6-[1-(2,2-diethoxyethyl)pyrazol-4-yl]-1-fluoro-2-naphthyl]-1,1-dioxo-1,2,5-thiadiazolidin-3-one (5)

Into a 50 mL pressure tube containing a well-stirred solution of 5-(3-benzyloxy-6-bromo-1-fluoro-2-naphthyl)-1,1-dioxo-1,2,5-thiadiazolidin-3-one (4, 525 mg, 1.13 mmol) and 1-(2,2-diethoxyethyl)-4-(4,4,5,5-tetramethyl-1,3,2-dioxaborolan-2-yl)-1H-pyrazole (3, 541.67 mg, 1.47 mmol, formic acid salt) in 1,4-dioxane (12 mL) and water (3 mL) was added cesium carbonate (1.10 g, 3.38 mmol). The reaction mixture was purged with nitrogen for 10 min, then PdCl₂(dtbpf) (51.48 mg, 78.98 μmol) was added. The reaction mixture was heated to 90° C. After 16 h, the reaction mixture was filtered through celite and washed with ethyl acetate (20 mL). The filtrate was diluted with water (15 mL) and extracted with 10% MeOH in EtOAc (2×50 mL). The combined organic layer was washed with brine solution (15 mL), dried over anhydrous Na₂SO₄, filtered and solvent removed under reduced pressure. The residue was triturated with diethyl ether (50 mL), filtered and dried to afford of 5-[3-benzyloxy-6-[1-(2,2-diethoxyethyl)pyrazol-4-yl]-1-fluoro-2-naphthyl]-1,1-dioxo-1,2,5-thiadiazolidin-3-one (5, 600 mg, 85% yield) as a brown solid. LCMS (ES+): m/z 569.0 [M+H]⁺

### Step 3: 2-[4-[7-benzyloxy-5-fluoro-6-(1,1,4-trioxo-1,2,5-thiadiazolidin-2-yl)-2-naphthyl]pyrazol-1-yl]acetaldehyde (6)

Into a 50 mL single neck round bottom flask containing a well-stirred solution of 5-[3-benzyloxy-6-[1-(2,2-diethoxyethyl)pyrazol-4-yl]-1-fluoro-2-naphthyl]-1,1-dioxo-1,2,5-thiadiazolidin-3-one (5, 300 mg, 481.17 μmol) in anhydrous DCM (15 mL) was added trifluoroacetic acid (2.74 g, 24.06 mmol, 1.85 mL) at 0° C. The resulting solution was stirred at RT. After 16 h, the volatiles were removed and the residue was triturated with diethyl ether (10 mL), filtered and dried to afford 2-[4-[7-benzyloxy-5-fluoro-6-(1,1,4-trioxo-1,2,5-thiadiazolidin-2-yl)-2-naphthyl]pyrazol-1-yl]acetaldehyde (6, 270 mg, 87% yield, TFA salt) as a brown solid. LCMS (ES+): m/z 494.9 [M+H]⁺

### 1-[7-benzyloxy-5-fluoro-6-(1,1,4-trioxo-1,2,5-thiadiazolidin-2-yl)-2-naphthyl]pyrazole-3-carbaldehyde (5)

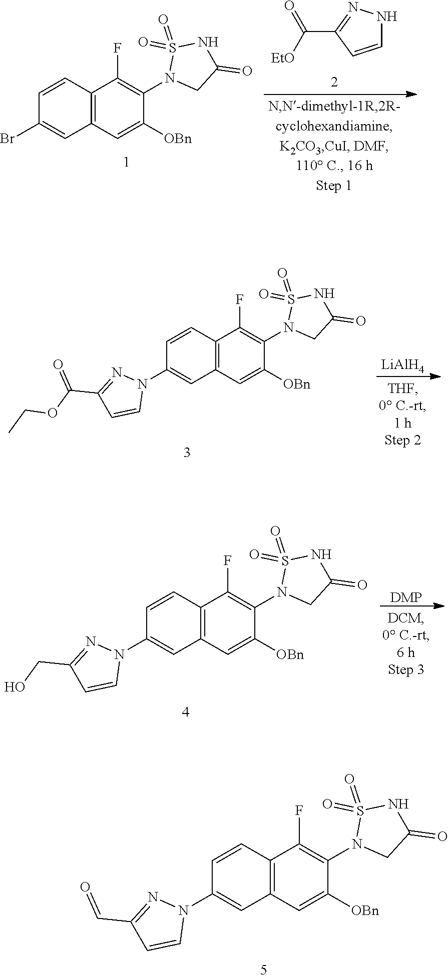

### Step 1: Ethyl 1-[7-benzyloxy-5-fluoro-6-(1,1,4-trioxo-1,2,5-thiadiazolidin-2-yl)-2-naphthyl]pyrazole-3-carboxylate (3)

Into a 50 mL pressure tube containing a well-stirred solution of 5-(3-benzyloxy-6-bromo-1-fluoro-2-naphthyl)-1,1-dioxo-1,2,5-thiadiazolidin-3-one (1, 1 g, 2.15 mmol) in DMF (10 mL) were added ethyl 1H-pyrazole-3-carboxylate (2, 451.78 mg, 3.22 mmol), copper (I) iodide (450.24 mg, 2.36 mmol), potassium carbonate (594.08 mg, 4.30 mmol, 259.42 μL) and (1R,2R)—N,N′-Dimethyl-1,2-cyclohexanediamine (226.22 mg, 1.59 mmol) at RT. The tube was sealed the reaction mixture was heated to 110° C. After 16 h, the reaction mixture was filtered through Celite and washed with ethyl acetate (100 mL). The filtrate was concentrated under reduced pressure and purified by reverse phase column chromatography [Purification method: Siliasep premium C18, 25 μm, 120) g column; Mobile phase: 0.1% TFA in water; Mobile phase B: Acetonitrile) to afford ethyl 1-[7-benzyloxy-5-fluoro-6-(1,1,4-trioxo-1,2,5-thiadiazolidin-2-yl)-2-naphthyl]pyrazole-3-carboxylate (3, 500 mg, 882.43 μmol, 41% yield) as a pale yellow solid. LCMS (ES+): m/z 525.0 [M+H]⁺

### Step 2: 5-[3-benzyloxy-1-fluoro-6-[3-(hydroxymethyl)pyrazol-1-yl]-2-naphthyl]-1,1-dioxo-1,2,5-thiadiazolidin-3-one (4)

Into a 100 mL two-neck round bottom flask containing a solution of ethyl 1-[7-benzyloxy-5-fluoro-6-(1,1,4-trioxo-1,2,5-thiadiazolidin-2-yl)-2-naphthyl]pyrazole-3-carboxylate (3, 500 mg, 772.13 μmol) in THF (15 mL) was added lithium aluminum hydride (2.0 M in THF) (1.16 mmol, 0.7 mL) at (° C.) The reaction mixture was stirred at ambient temperature. After 1 h, the reaction mixture was quenched with ice-water (20 mL) at (° C.) The volatiles were removed under reduced pressure. The material was purified by reverse phase column chromatography [Purification method: Siliasep C18 120 g column; Mobile phase: 0.1% TFA in water; Mobile phase B: MeCN] to afford 5-[3-benzyloxy-1-fluoro-6-[3-(hydroxymethyl)pyrazol-1-yl]-2-naphthyl]-1,1-dioxo-1,2,5-thiadiazolidin-3-one (4, 300 mg, 599.52 μmol, 78% yield) as a pale yellow solid. LCMS (ES+): m/z 483.1 [M+H]⁺

### Step 3: 1-[7-benzyloxy-5-fluoro-6-(1,1,4-trioxo-1,2,5-thiadiazolidin-2-yl)-2-naphthyl]pyrazole-3-carbaldehyde (5)

Into a 50 mL single neck round bottom flask containing a well-stirred solution of 5-[3-benzyloxy-1-fluoro-6-[3-(hydroxymethyl)pyrazol-1-yl]-2-naphthyl]-1,1-dioxo-1,2,5-thiadiazolidin-3-one (4, 300 mg, 596.91 μmol) in DCM (10 mL) was added Dess-Martin periodinane (379.76 mg, 895.37 μmol) at (° C.) The resultant solution was stirred at ambient temperature. After 6 h, the reaction mixture was concentrated under reduced pressure and the residue was taken up in MeCN (20 mL) and THF (20 mL) and filtered through Celite, washed with MeCN (40 mL). The filtrate was concentrated to dryness to obtain 1-[7-benzyloxy-5-fluoro-6-(1,1,4-trioxo-1,2,5-thiadiazolidin-2-yl)-2-naphthyl]pyrazole-3-carbaldehyde (5, 280) mg, 330.95 μmol, 55% yield, 57% purity) as a pale yellow solid. The material was used in the next step without further purification. LCMS (ES−): m/z 479.0 [M−H]⁻

### 5-(1-fluoro-3-hydroxy-7-piperazin-1-yl-2-naphthyl)-1,1-dioxo-1,2,5-thiadiazolidin-3-one (4)

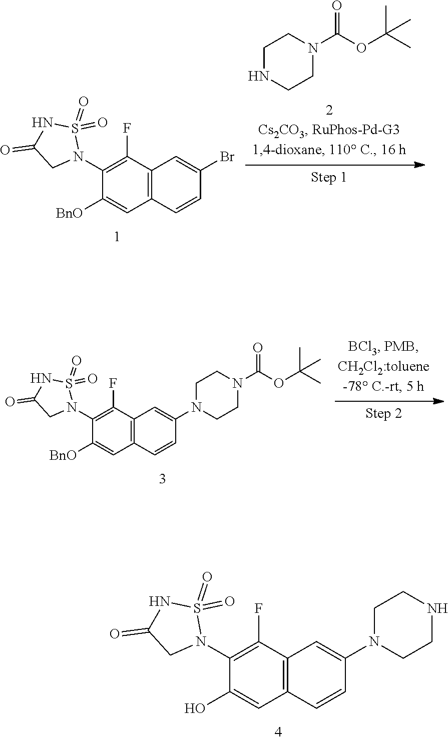 [SMILES: CC(C)(C)OC(=O)N1CCNCC1 ; O=C1CN(c2c(OCc3ccccc3)cc3ccc(Br)cc3c2F)S(=O)(=O)N1 ; CC(C)(C)OC(=O)N1CCN(c2ccc3cc(OCc4ccccc4)c(N4CC(=O)NS4(=O)=O)c(F)c3c2)CC1 ; O=C1CN(c2c(O)cc3ccc(N4CCNCC4)cc3c2F)S(=O)(=O)N1]

### Step 1: tert-butyl 4-[6-benzyloxy-8-fluoro-7-(1,1,4-trioxo-1,2,5-thiadiazolidin-2-yl)-2-naphthyl]piperazine-1-carboxylate (3)

Into a 50 mL pressure tube containing a well-stirred solution of 5-(3-benzyloxy-7-bromo-1-fluoro-2-naphthyl)-1,1-dioxo-1,2,5-thiadiazolidin-3-one (1, 300 mg, 644.76 μmol) in anhydrous 1,4-dioxane (8 mL) were added Cs₂CO₃ (252.09 mg, 773.73 μmol) and tert-butyl piperazine-1-carboxylate (2, 120.09 mg, 644.76 μmol). The suspension was degassed with nitrogen for 5 min. Then Ruphos Pd G3 (80.87 mg, 96.71 μmol) was added and degassed with nitrogen for 5 min. The tube was sealed the reaction mixture was heated at 110° C. After 16 h, the reaction mixture was filtered through Celite and washed with ethyl acetate (100 mL). The filtrate was concentrated and the residue was purified by reverse phase column chromatography [Purification method: Siliasep C18 120 g column; Mobile phase A: 0.1% TFA in water; Mobile phase B: MeCN] to afford tert-butyl 4-[6-benzyloxy-8-fluoro-7-(1,1,4-trioxo-1,2,5-thiadiazolidin-2-yl)-2-naphthyl]piperazine-1-carboxylate (3, 200 mg, 254.81 μmol, 40% yield) as a pale yellow solid. LCMS (ES+): m/z 571.2 [M+H]⁺

### Step 2: 5-(1-fluoro-3-hydroxy-7-piperazin-1-yl-2-naphthyl)-1,1-dioxo-1,2,5-thiadiazolidin-3-one (4)

Into a 50 mL single neck round bottom flask containing a well-stirred solution of tert-butyl 4-[6-benzyloxy-8-fluoro-7-(1,1,4-trioxo-1,2,5-thiadiazolidin-2-yl)-2-naphthyl]piperazine-1-carboxylate (3, 170 mg, 214.50 μmol) in DCM (5 mL) and toluene (5 mL) was added pentamethylbenzene (140.29 mg, 946.32 μmol) and the solution was cooled to −75° C. Then boron trichloride (1.0 M in DCM) (4.29 mmol, 4.5 mL) was added at −75° C. The reaction mixture was then stirred at RT. After 5 h, the reaction mixture was quenched with 5% MeOH in DCM (2 mL) at −75° C. The reaction mixture was concentrated under reduced pressure and the residue was triturated with diethyl ether (10 mL), filtered and dried to obtain 5-(1-fluoro-3-hydroxy-7-piperazin-1-yl-2-naphthyl)-1,1-dioxo-1,2,5-thiadiazolidin-3-one (4, 90 mg, 210.62 μmol, 98% yield) as a pale yellow solid. LCMS (ES+): m/z 381.1[M+H]⁺

### N-(7-(benzyloxy)-6-(1,1-dioxido-4-oxo-1,2,5-thiadiazolidin-2-yl)-5-fluoronaphthalen-2-yl)-3-bromopropanamide (2)

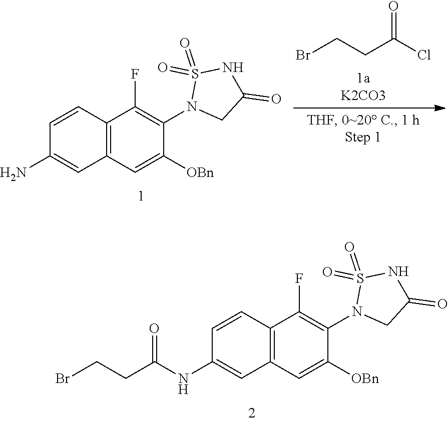 [SMILES: Nc1ccc2c(F)c(N3CC(=O)NS3(=O)=O)c(OCc3ccccc3)cc2c1 ; O=C(Cl)CCBr ; O=C(CCBr)Nc1ccc2c(F)c(N3CC(=O)NS3(=O)=O)c(OCc3ccccc3)cc2c1]

### Step 1: N-(7-(benzyloxy)-6-(1,1-dioxido-4-oxo-1,2,5-thiadiazolidin-2-yl)-5-fluoronaphthalen-2-yl)-3-bromopropanamide (2)

To a mixture of 5-(6-amino-3-(benzyloxy)-1-fluoronaphthalen-2-yl)-1,2,5-thiadiazolidin-3-one 1,1-dioxide (1, 100 mg, 194.01 μmol, TFA salt) and K₂CO₃ (67.03 mg, 485.03 μmol, 29.27 μL) in THF (5 mL) was added 3-bromopropanoyl chloride (1a, 33.26 mg, 194.01 μmol) at 0° C. After 1 h at RT, the mixture was filtered and concentrated under reduced pressure to afford N-(7-(benzyloxy)-6-(1,1-dioxido-4-oxo-1,2,5-thiadiazolidin-2-yl)-5-fluoronaphthalen-2-yl)-3-bromopropanamide (2, 104 mg, 193.90 μmol, 100% yield) as yellow solid. The material was used in the next step without further purification. LCMS (ESI): m/z 535.9/537.9 [M+H]⁺

### 5-(7-(azetidin-3-ylamino)-1-fluoro-3-hydroxynaphthalen-2-yl)-1,2,5-thiadiazolidin-3-one 1,1-dioxide (4)

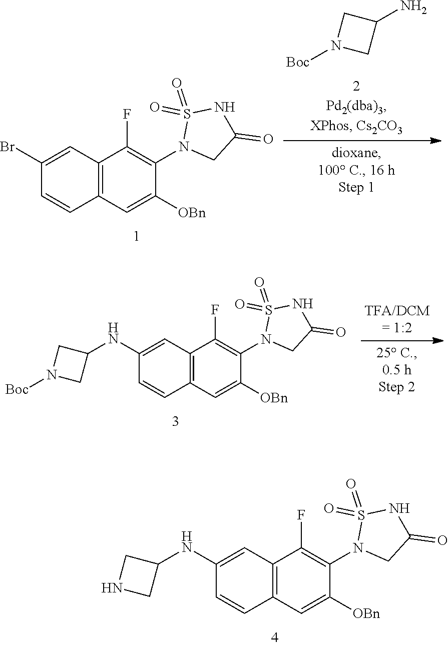 [SMILES: CC(C)(C)OC(=O)N1CC(N)C1 ; O=C1CN(c2c(OCc3ccccc3)cc3ccc(Br)cc3c2F)S(=O)(=O)N1 ; CC(C)(C)OC(=O)N1CC(Nc2ccc3cc(OCc4ccccc4)c(N4CC(=O)NS4(=O)=O)c(F)c3c2)C1 ; O=C1CN(c2c(OCc3ccccc3)cc3ccc(NC4CNC4)cc3c2F)S(=O)(=O)N1]

### Step 1: tert-butyl 3-((6-(benzyloxy)-7-(1,1-dioxido-4-oxo-1,2,5-thiadiazolidin-2-yl)-8-fluoronaphthalen-2-yl)amino)azetidine-1-carboxylate (3)

To a solution of 5-(3-(benzyloxy)-7-bromo-1-fluoronaphthalen-2-yl)-1,2,5-thiadiazolidin-3-one 1,1-dioxide (1, 500 mg, 1.07 mmol) and tert-butyl3-aminoazetidine-1-carboxylate (2, 370 mg, 2.15 mmol) in dioxane (10 mL) were added cesium carbonate (1.05 g, 3.22 mmol), dicyclohexyl-[2-(2,4,6-triisopropylphenyl)phenyl]phosphane (51 mg, 106.98 μmol) and (1E,4E)-1,5-diphenylpenta-1,4-dien-3-one: palladium (49 mg, 53.51 μmol). The mixture was heated to 100° C. and stirred for 16 h under N₂. The mixture was filtered. The filtrate was purified by reversed phase chromatography (C18, 0.1% formic acid in water/MeCN) to afford tert-butyl 3-((6-(benzyloxy)-7-(1,1-dioxido-4-oxo-1,2,5-thiadiazolidin-2-yl)-8-fluoronaphthalen-2-yl)amino)azetidine-1-carboxy late (3, 350 mg, 609.95 μmol, 58% yield) as an off-white solid. LCMS (ESI): m/z 501.2 (M+H-1-Bu)⁺

### Step 2: 5-(7-(azetidin-3-ylamino)-1-fluoro-3-hydroxynaphthalen-2-yl)-1,2,5-thiadiazolidin-3-one 1,1-dioxide (4)

A mixture of tert-butyl 3-((6-(benzyloxy)-7-(1,1-dioxido-4-oxo-1,2,5-thiadiazolidin-2-yl)-8-fluoronaphthalen-2-yl)amino)azetidine-1-carboxy late (3, 360 mg, 595.04 mol) and TFA (7.40 g, 64.90 mmol, 5 mL) in DCM (10 mL) was stirred for 0.5 h. The mixture was concentrated under reduced pressure to afford a residue which was azeotroped twice with toluene (2×20 mL) to afford 5-(7-(azetidin-3-ylamino)-1-fluoro-3-hydroxynaphthalen-2-yl)-1,2,5-thiadiazolidin-3-one 1,1-dioxide (4, 400 mg, 595.96 μmol, 100% yield, TFA salt) as a brown solid. The material was used into next step without further purification. LCMS (ESI): m/z 367.1 [M+H]⁺

### 3-[4-[1-(2,6-dioxo-3-piperidyl)-3-methyl-2-oxo-benzimidazol-5-yl]-1-piperidyl]cyclobutanecarboxylic acid (4)

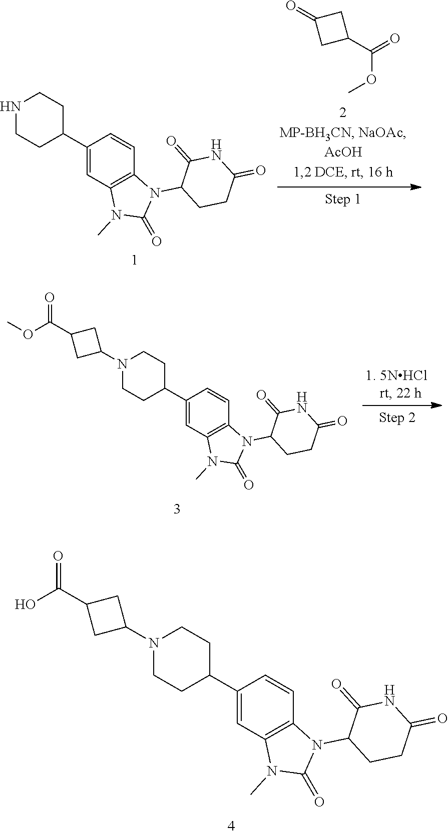

### Step 1: methyl 3-[4-[1-(2,6-dioxo-3-piperidyl)-3-methyl-2-oxo-benzimidazol-5-yl]-1-piperidyl]cyclobutanecarboxylate (3)

Into a 10 mL single neck round bottom flask containing a well-stirred suspension of 3-[3-methyl-2-oxo-5-(4-piperidyl)benzimidazol-1-yl]piperidine-2,6-dione (1, 500 mg, 1.32 mmol, HCl salt) and methyl 3-oxocyclobutanecarboxylate (2, 169.10 mg, 1.32 mmol) in 1,2-DCE (10 mL) was added sodium acetate (541.33 mg, 6.60 mmol) followed by acetic acid (792.55 mg, 13.20 mmol, 754.81 μL). After 30 min, MP-cyanoborohydride (700 mg, 1.4 mmol) was added. After 16 h, the reaction mixture was diluted with DCM (20 mL) and filtered through Celite. The filtrate was concentrated under reduced pressure to afford methyl 3-[4-[1-(2,6-dioxo-3-piperidyl)-3-methyl-2-oxo-benzimidazol-5-yl]-1-piperidyl]cyclobutanecarboxylate (3, 500 mg, 913.06 μmol, 69% yield) as an off-white solid. LCMS (ES+): m/z 455.2 (M+H)⁺

### Step 2: 3-[4-[1-(2,6-dioxo-3-piperidyl)-3-methyl-2-oxo-benzimidazol-5-yl]-1-piperidyl]cyclobutanecarboxylic acid (4)

Into a 10 mL round bottom flask containing a well-stirred suspension of methyl methyl 3-[4-[1-(2,6-dioxo-3-piperidyl)-3-methyl-2-oxo-benzimidazol-5-yl]-1-piperidyl]cyclobutanecarboxylate (3, 500 mg, 0.913 mmol) was added 1.5 N HCl (5 mL). After 22 h, the reaction mixture was lyophilized to afford 3-[4-[1-(2,6-dioxo-3-piperidyl)-3-methyl-2-oxo-benzimidazol-5-yl]-1-piperidyl]cyclobutanecarboxylic acid (4, 490 mg, 821.89 μmol, 90% yield, HCl salt) as a colorless solid. The material was used in the next step without purification. LCMS (ES+): m/z 441.1(M+H)⁺

### 2-[4-[1-[(3R)-2,6-dioxo-3-piperidyl]-3-methyl-2-oxo-benzimidazol-5-yl]-1-piperidyl]acetic acid (5)

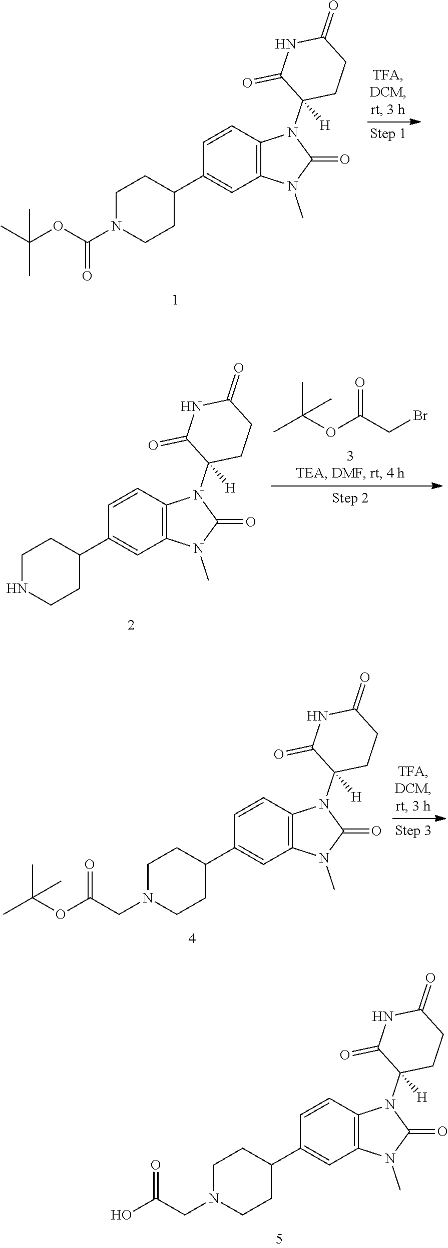

### Step 1: (3R)-3-[3-methyl-2-oxo-5-(4-piperidyl)benzimidazol-1-yl]piperidine-2,6-dione (2)

Into a 25 mL single neck round bottom flask containing a well-stirred solution of tert-butyl 4-[1-[(3R)-2,6-dioxo-3-piperidyl]-3-methyl-2-oxo-benzimidazol-5-yl]piperidine-1-carboxylate (1, 100 mg, 225.99 μmol) in DCM (3 mL) was added TFA (25.77 mg, 2 mL) at 0° C. After 3 h at RT, the volatiles were removed under reduced pressure, triturated with Et₂O (30 mL) and filtered and dried to afford (3R)-3-[3-methyl-2-oxo-5-(4-piperidyl)benzimidazol-1-yl]piperidine-2,6-dione (2, 94 mg, 205.34 μmol, 91% yield, TFA salt) as an off-white solid. LCMS (ES+): m/z 343.1 [M+H]⁺

### Step 2: tert-butyl 2-[4-[1-[(3R)-2,6-dioxo-3-piperidyl]-3-methyl-2-oxo-benzimidazol-5-yl]-1-piperidyl]acetate (4)

Into a 10 mL single neck round bottom flask containing a well-stirred solution of (3R)-3-[3-methyl-2-oxo-5-(4-piperidyl)benzimidazol-1-yl]piperidine-2,6-dione (2, 90 mg, 197.19 μmol, TFA salt) in DMF (2 mL) was added TEA (99.77 mg, 985.95 μmol, 137.42 μL) followed by tert-butyl 2-bromoacetate (3, 57.69 mg, 295.78 μmol, 43.38 μL) dropwise at 0° C. The reaction mixture was stirred at RT for 4 h. The reaction was quenched with water (5 mL) and extracted with EtOAc (3×5 mL). The combined organic layer was washed with brine solution (10 mL) and concentrated under reduced pressure to afford tert-butyl 2-[4-[1-[(3R)-2,6-dioxo-3-piperidyl]-3-methyl-2-oxo-benzimidazol-5-yl]-1-piperidyl]acetate (4, 86 mg, 184.36 μmol, 94% yield) as an off white solid. LCMS (ES+): m/z 457.3 [M+H]⁺

### Step 3: 2-[4-[1-[(3R)-2,6-dioxo-3-piperidyl]-3-methyl-2-oxo-benzimidazol-5-yl]-1-piperidyl]acetic acid (5)

Into a 10 mL single neck round bottom flask containing a well-stirred solution of tert-butyl 2-[4-[1-[(3R)-2,6-dioxo-3-piperidyl]-3-methyl-2-oxo-benzimidazol-5-yl]-1-piperidyl]acetate (4, 80 mg, 171.50 μmol) in DCM (2 mL) was added TFA (1.5 mL) at 0° C. slowly. The reaction mixture was stirred at RT for 3 h. The volatiles were removed under reduced pressure, triturated with Et₂O (5 mL), filtered and dried to afford 2-[4-[1-[(3R)-2,6-dioxo-3-piperidyl]-3-methyl-2-oxo-benzimidazol-5-yl]-1-piperidyl]acetic acid (5, 68 mg, 130.02 μmol, 76% yield, TFA salt) as an off-white solid. LCMS (ES+): m/z 401.1 [M+H]⁺

### 2-[4-[4-[(2,6-dioxo-3-piperidyl)-methyl-amino]phenyl]-1-piperidyl]acetic acid (4)

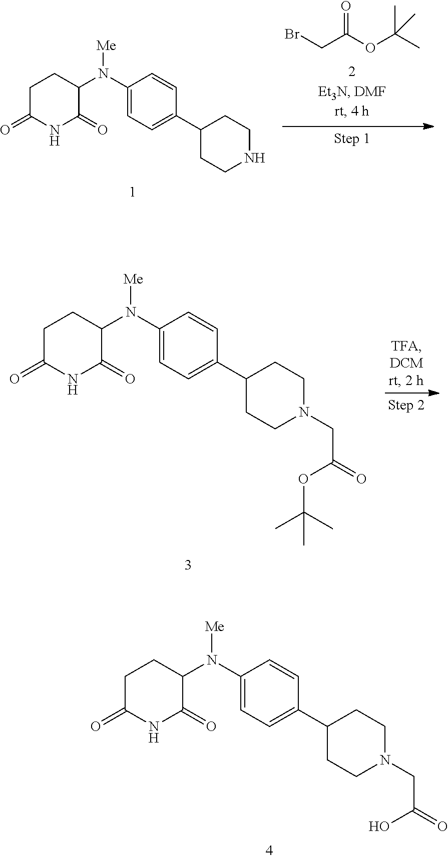

### Step 1: tert-butyl 2-[4-[4-[(2,6-dioxo-3-piperidyl)-methyl-amino]phenyl]-1-piperidyl]acetate (3)

Into a 50 mL single neck round bottom flask containing a well-stirred solution of 3-[N-methyl-4-(4-piperidyl)anilino]piperidine-2,6-dione (1, 220 mg, 729.97 μmol, HCl salt) in anhydrous DMF (3 mL) were added triethylamine (221.60 mg, 2.19 mmol, 305.23 μL) and tert-butyl-2-bromoacetate (2, 170.86 mg, 875.96 μmol, 128.47 μL) at 0° C. After 1 h at RT, the reaction mixture was diluted with water (15 mL) and extracted with EtOAc (25 mL×2). The combined organic layer was washed with brine solution (25 mL), dried over anhydrous sodium sulfate and filtered. The solvent was removed under reduced pressure to obtain tert-butyl 2-[4-[4-[(2,6-dioxo-3-piperidyl)-methyl-amino]phenyl]-1-piperidyl]acetate (3, 280 mg, 87% yield). The material was used in the next step without purification. LCMS (ES+): m/z 416.4 [M+H]⁺

### Step 2: 2-[4-[4-[(2,6-dioxo-3-piperidyl)-methyl-amino]phenyl]-1-piperidyl]acetic acid (4)

Into a 50 mL single neck round bottom flask containing a well-stirred solution of tert-butyl 2-[4-[4-[(2,6-dioxo-3-piperidyl)-methyl-amino]phenyl]-1-piperidyl]acetate (3, 280 mg, 633.42 μmol) in anhydrous DCM (10 mL) was added TFA (7.40 g, 64.90 mmol, 5 mL). After 2 h, the volatiles were removed and the residue was triturated with diethyl ether (10 mL×2), filtered and dried to afford 2-[4-[4-[(2,6-dioxo-3-piperidyl)-methyl-amino]phenyl]-1-piperidyl]acetic acid (4, 200 mg, 65% yield, TFA salt) as a brown solid. LCMS (ES+): m/z 360.2 [M+H]⁺

### 2-[4-[4-[(2,6-dioxo-3-piperidyl)amino]-3-phenoxy-phenyl]-1-piperidyl]acetic acid (8)

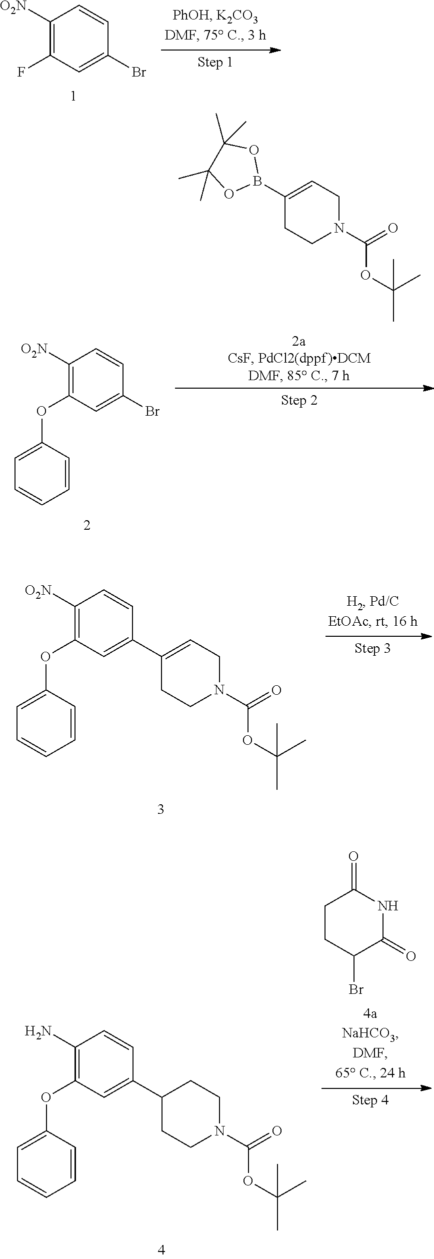 [SMILES: O=[N+]([O-])c1ccc(Br)cc1F ; CC(C)(C)OC(=O)N1CC=C(B2OC(C)(C)C(C)(C)O2)CC1 ; O=[N+]([O-])c1ccc(Br)cc1Oc1ccccc1 ; CC(C)(C)OC(=O)N1CC=C(c2ccc([N+](=O)[O-])c(Oc3ccccc3)c2)CC1 ; O=C1CCC(Br)C(=O)N1 ; CC(C)(C)OC(=O)N1CCC(c2ccc(N)c(Oc3ccccc3)c2)CC1]

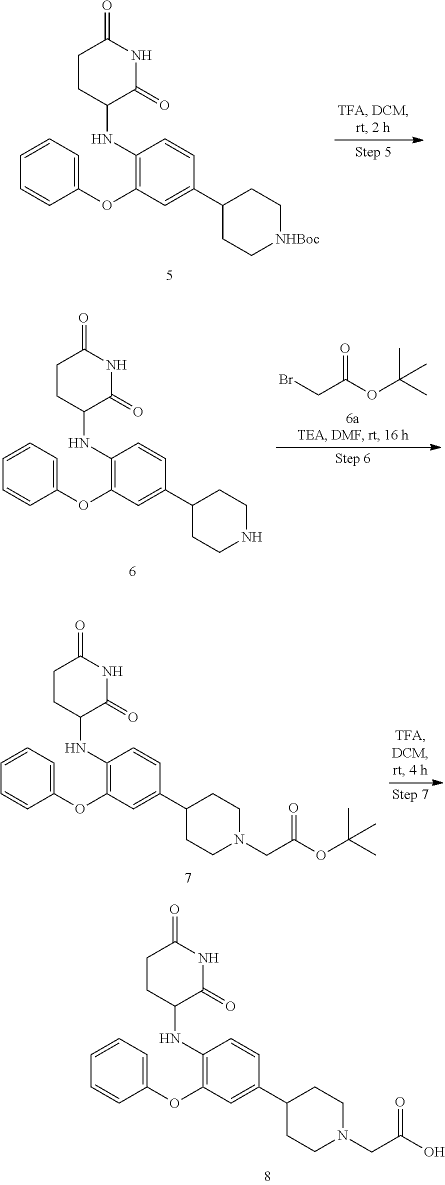

### Step 1: 4-bromo-1-nitro-2-phenoxy-benzene (2)

Into a 50 mL pressure tube containing a well-stirred solution of 4-bromo-2-fluoro-1-nitro-benzene (1, 3 g, 13.64 mmol) in DMF (20 mL) was added phenol (1.28 g, 13.64 mmol, 1.20 mL) and potassium carbonate (3.77 g, 27.27 mmol, 1.65 mL) at 0° C. and the resulting mixture was stirred at 75° C. for 3 h. The reaction mixture was cooled to 0° C. and ice water (100 mL) was added. The precipitate was filtered, washed with water and dried under reduced pressure to afford 4-bromo-1-nitro-2-phenoxy-benzene (2, 3.8 g, 12.02 mmol, 88% yield) as a yellow solid.

¹H NMR (400 MHZ, DMSO-d₆): δ 8.05 (d, J=11.60 Hz, 1H), 7.59 (dd, J=2.40, 11.80 Hz, 1H), 7.50-7.44 (m, 2H), 7.29-7.24 (m, 2H), 7.16-7.13 (m, 2H).

### Step 2: tert-butyl 4-(4-nitro-3-phenoxy-phenyl)-3,6-dihydro-2H-pyridine-1-carboxylate (3)

Into a 50 mL pressure tube containing a well-stirred solution of 4-bromo-1-nitro-2-phenoxy-benzene (2, 1 g, 3.16 mmol) in anhydrous DMF (12 mL) was added tert-butyl 4-(4,4,5,5-tetramethyl-1,3,2-dioxaborolan-2-yl)-3,6-dihydro-2H-pyridine-1-carboxylate (2a, 1.47 g, 4.74 mmol) and cesium fluoride (1.20 g, 7.91 mmol, 291.47 μL) and the reaction mixture was purged with nitrogen for 10 min. Then. Pd(dppf)Cl₂·CH₂Cl₂ (387.35 mg, 474.33 μmol) was added and the resulting mixture was heated at 85° C. for 7 h. The reaction mixture was cooled and filtered through Celite, washing with ethyl acetate (150 mL). The filtrate was washed with water (100) mL) followed by brine solution (50 mL), dried over Na₂SO₄, filtered and concentrated under reduced pressure. The material was purified by flash silica gel column chromatography (20-30% of ethyl acetate in pet-ether) to obtain tert-butyl 4-(4-nitro-3-phenoxy-phenyl)-3,6-dihydro-2H-pyridine-1-carboxylate (3, 1.2 g, 2.98 mmol, 94% yield) as a brown liquid. LCMS (ES−): m/z 395.0 [M−H]⁻

### Step 3: tert-butyl 4-(4-amino-3-phenoxy-phenyl)piperidine-1-carboxylate (4)

Into a 100 mL single neck round bottom flask containing a well-stirred solution of tert-butyl 4-(4-nitro-3-phenoxy-phenyl)-3,6-dihydro-2H-pyridine-1-carboxylate (3, 1 g, 2.48 mmol) in ethyl acetate (30 mL) was added palladium on carbon (10%) (528.62 mg, 4.97 mmol). The reaction mixture was stirred under hydrogen atmosphere at RT for 16 h. The reaction mixture was filtered through Celite and washed with ethyl acetate (300 mL). The filtrate was concentrated under reduced pressure to obtain tert-butyl 4-(4-amino-3-phenoxy-phenyl)piperidine-1-carboxylate (4, 900 mg, 2.36 mmol, 95% yield) as a brown solid. LCMS (ES+): m/z 313.2 [M-tBu+H]⁺

### Step 4: tert-butyl 4-[4-[(2,6-dioxo-3-piperidyl)amino]-3-phenoxy-phenyl]piperidine-1-carboxylate (5)

Into a 20 mL vial containing a well-stirred solution of tert-butyl 4-(4-amino-3-phenoxy-phenyl)piperidine-1-carboxylate (4, 700 mg, 1.82 mmol) in DMF (10 mL) was added sodium bicarbonate (306.42 mg, 3.65 mmol, 141.86 μL) at (° C.) After 10 min. 3-bromopiperidine-2,6-dione (4a, 525.27 mg, 2.74 mmol) was added and the resulting mixture was heated at 65° C. for 25 h. The reaction was quenched with ice water (30 mL) and extracted with ethyl acetate (2×100 mL). The combined organic layer was washed with water (150 mL) followed by brine solution (100 mL) and dried over sodium sulfate. The solvent was removed under reduced pressure and the residue was purified by flash silica gel column chromatography (30-40% ethyl acetate in pet-ether) to afford tert-butyl 4-[4-[(2,6-dioxo-3-piperidyl)amino]-3-phenoxy-phenyl]piperidine-1-carboxylate (5, 295 mg, 605.73 μmol, 33% yield) as a green solid. LCMS (ES+): m/z 424.2 [M-tBu+H]⁺

### Step 5: 3-[2-phenoxy-4-(4-piperidyl)anilino]piperidine-2,6-dione (6)

Into a 25 mL single neck round bottom flask containing a well-stirred suspension of tert-butyl 4-[4-[(2,6-dioxo-3-piperidyl)amino]-3-phenoxy-phenyl]piperidine-1-carboxylate (5, 295 mg, 602.84 μmol) in DCM (8 mL) was added trifluoroacetic acid (687.37 mg, 6.03 mmol, 464.44 μL) dropwise at (° C.) After 2 h at RT, the volatiles were removed under reduced pressure and the residue was triturated with diethyl ether (10 mL), filtered and dried to afford 3-[2-phenoxy-4-(4-piperidyl)anilino]piperidine-2,6-dione (6, 290 mg, 505.40 μmol, 84% yield, TFA salt) as a green solid. LCMS (ES+): m/z 380.2 [M+H]⁺

### Step 6: tert-butyl 2-[4-[4-[(2,6-dioxo-3-piperidyl)amino]-3-phenoxy-phenyl]-1-piperidyl]acetate (7)

Into a 8 mL vial containing a well-stirred solution of 3-[2-phenoxy-4-(4-piperidyl)anilino]piperidine-2,6-dione (6, 290 mg, 505.40 μmol, TFA salt) in DMF (5 mL) was added triethylamine (153.42 mg, 1.52 mmol, 211.33 μL). After 5 min, tert-butyl 2-bromoacetate (6a, 118.30 mg, 606.48 μmol, 88.94 μL) was added. After 16 h the reaction mixture was poured into ice water (50 mL). The precipitate was filtered, washed with water and dried under vacuum to afford tert-butyl 2-[4-[4-[(2,6-dioxo-3-piperidyl)amino]-3-phenoxy-phenyl]-1-piperidyl]acetate (7, 270) mg, 482.46 μmol, 95% yield) as a green solid. LCMS (ES+): m/z 494.3 [M+H]⁺

### Step 7: 2-[4-[4-[(2,6-dioxo-3-piperidyl)amino]-3-phenoxy-phenyl]-1-piperidyl]acetic acid (8)

Into a 25 mL single neck round bottom flask containing a well-stirred suspension of tert-butyl 2-[4-[4-[(2,6-dioxo-3-piperidyl)amino]-3-phenoxy-phenyl]-1-piperidyl]acetate (7, 270 mg, 482.46 μmol) in DCM (5 mL) was added trifluoroacetic acid (550.10 mg, 4.82 mmol, 371.69 μL) dropwise at (° C.) The resulting mixture was stirred at RT for 4 h. The volatiles were removed under reduced pressure and the residue was triturated with diethyl ether (10 mL), filtered and dried to afford 2-[4-[4-[(2,6-dioxo-3-piperidyl)amino]-3-phenoxy-phenyl]-1-piperidyl]acetic acid (8, 265 mg, 471.37 μmol, 98% yield, TFA salt) as a grey solid. LCMS (ES+): m/z 438.2 [M+H]⁺

### 3-[5-[4-(aminomethyl)-1-piperidyl]-3-methyl-2-oxo-benzimidazol-1-yl]piperidine-2,6-dione (5)

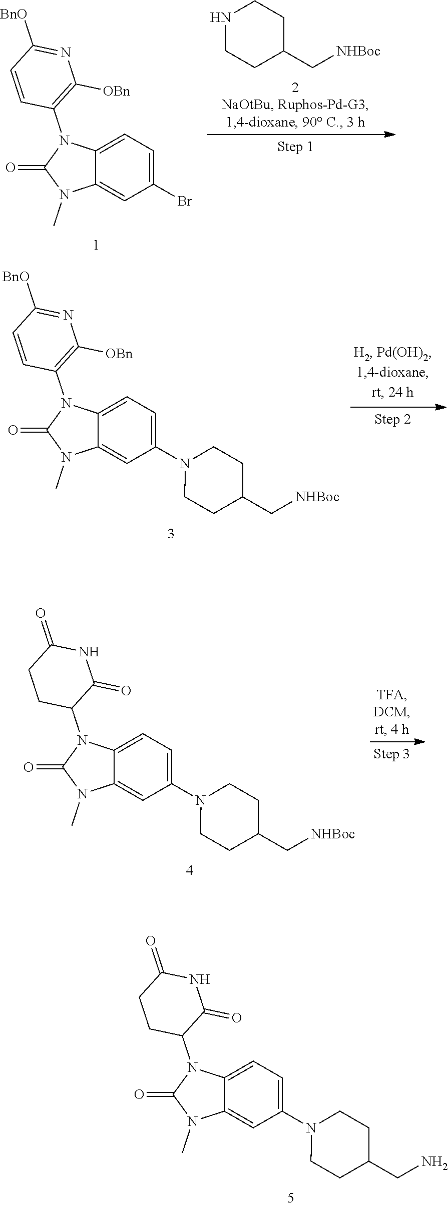 [SMILES: Cn1c(=O)n(-c2ccc(OCc3ccccc3)nc2OCc2ccccc2)c2ccc(Br)cc21 ; CC(C)(C)OC(=O)NCC1CCNCC1 ; Cn1c(=O)n(-c2ccc(OCc3ccccc3)nc2OCc2ccccc2)c2ccc(N3CCC(CNC(=O)OC(C)(C)C)CC3)cc21 ; Cn1c(=O)n(C2CCC(=O)NC2=O)c2ccc(N3CCC(CNC(=O)OC(C)(C)C)CC3)cc21 ; Cn1c(=O)n(C2CCC(=O)NC2=O)c2ccc(N3CCC(CN)CC3)cc21]

### Step 1: tert-butyl N-[[1-[1-(2,6-dibenzyloxy-3-pyridyl)-3-methyl-2-oxo-benzimidazol-5-yl]-4-piperidyl]methyl]carbamate (3)

Into a 10 mL pressure tube containing well-stirred solution of 5-bromo-1-(2,6-dibenzyloxy-3-pyridyl)-3-methyl-benzimidazol-2-one (1, 500 mg, 968.27 μmol) and tert-butyl N-(4-piperidylmethyl)carbamate (2, 269.75 mg, 1.26 mmol) in anhydrous 1,4-dioxane (6 mL) were added RuPhos Pd G3 (80.98 mg, 96.83 μmol) and sodium tert-butoxide (279.16 mg, 2.90 mmol). The mixture was purged with nitrogen gas for 15 min. After 3 h at 90° C., the reaction mixture was filtered through Celite, concentrated and partitioned between ethyl acetate (50 mL) and water (20 mL). The organic layer was washed with water (20 mL), dried over anhydrous Na₂SO₄, and filtered. The filtrate was concentrated under reduced pressure and purified by flash silica gel (60-120 mesh) column chromatography (70% EtOAc in pet ether) to afford the tert-butyl N-[[1-[1-(2,6-dibenzyloxy-3-pyridyl)-3-methyl-2-oxo-benzimidazol-5-yl]-4-piperidyl]methyl]carbamate (3, 350 mg, 522.22 μmol, 54% yield) as light yellow solid. LCMS (ES+): m/z 650.6 [M+H]⁺

### Step 2: tert-butyl N-[[1-[1-(2,6-dioxo-3-piperidyl)-3-methyl-2-oxo-benzimidazol-5-yl]-4-piperidyl]methyl]carbamate (4)

Into a 50 mL single neck round bottom flask containing well-stirred solution of tert-butyl N-[[1-[1-(2,6-dibenzyloxy-3-pyridyl)-3-methyl-2-oxo-benzimidazol-5-yl]-4-piperidyl]methyl]carbamate (3, 350 mg, 522.22 μmol) in anhydrous 1,4-dioxane (10 mL) was added palladium hydroxide on carbon (20 wt. %, 50% water) (300 mg, 427.24 μmol, 20% purity) at RT. The suspension was stirred at RT for 24 h under hydrogen atmosphere (bladder). The reaction mixture was filtered through Celite and concentrated under reduced pressure to afford tert-butyl N-[[1-[1-(2,6-dioxo-3-piperidyl)-3-methyl-2-oxo-benzimidazol-5-yl]-4-piperidyl]methyl]carbamate (4, 230 mg, 486.88 μmol, 93% yield) as an off-white solid. The material was used in the next step without further purification. LCMS (ES+): m/z 472.2 [M+H]⁺

### Step 3: 3-[5-[4-(aminomethyl)-1-piperidyl]-3-methyl-2-oxo-benzimidazol-1-yl]piperidine-2,6-dione (5)

Into a 50 mL single neck round bottom flask containing well-stirred solution of tert-butyl N-[[1-[1-(2,6-dioxo-3-piperidyl)-3-methyl-2-oxo-benzimidazol-5-yl]-4-piperidyl]methyl]carbamate (4, 230 mg, 482.88 μmol) in anhydrous DCM (7 mL) was added TFA (2.22 g, 19.47 mmol, 1.5 mL). After 3 h, the solvent was removed under reduced pressure. The residue was triturated with diethyl ether (50 mL) to afford 3-[5-[4-(aminomethyl)-1-piperidyl]-3-methyl-2-oxo-benzimidazol-1-yl]piperidine-2,6-dione (5, 230 mg, 455.68 μmol, 94% yield, TFA salt) as an off-white solid. LCMS (ES+): m/z 372.2 [M+H]⁺

### 3-[5-[4-(aminomethyl)cyclohexen-1-yl]-3-methyl-2-oxo-benzimidazol-1-yl]piperidine-2,6-dione (5)

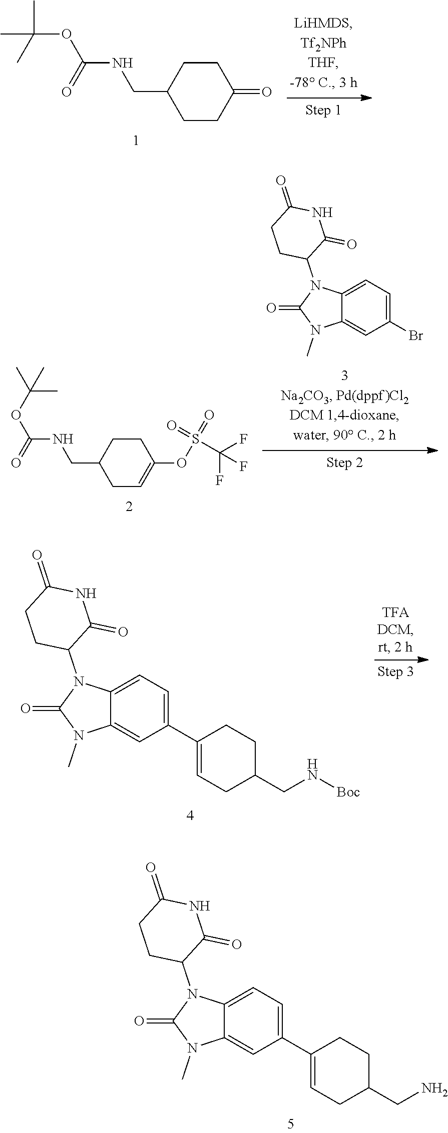 [SMILES: CC(C)(C)OC(=O)NCC1CCC(=O)CC1 ; Cn1c(=O)n(C2CCC(=O)NC2=O)c2ccc(Br)cc21 ; CC(C)(C)OC(=O)NCC1CC=C(OS(=O)(=O)C(F)(F)F)CC1 ; Cn1c(=O)n(C2CCC(=O)NC2=O)c2ccc(C3=CCC(CNC(=O)OC(C)(C)C)CC3)cc21 ; Cn1c(=O)n(C2CCC(=O)NC2=O)c2ccc(C3=CCC(CN)CC3)cc21]

### Step 1: [4-[(tert-butoxycarbonylamino)methyl]cyclohexen-1-yl] trifluoromethanesulfonate (2)

Into a 250 mL two neck round bottom flask containing a solution of lithium bis(trimethylsilyl)amide (1.0 M in THF) (5.41 g, 32.34 mmol) in THF (20 mL) was added a solution of tert-butyl N-[(4-oxocyclohexyl)methyl]carbamate (1, 3.5 g, 15.40 mmol) in THF (35 mL) at −78° C. dropwise. After 1.5 h, a solution of N-phenyl-bis(trifluoromethanesulfonimide) (5.50 g, 15.40 mmol) in THF (35 mL) was added slowly at −78° C. Then the reaction mixture was stirred at 5° C. for 3 h. The reaction was quenched with saturated NH₄Cl solution (100 mL) at 0° C. and the aqueous phase was extracted with diethyl ether (2×250 mL). The organic layer was washed with aq. NaHCO₃ solution (200 mL) followed by brine solution (200 mL). The organic layer was dried over anhydrous Na₂SO₄, filtered and concentrated under reduced pressure. The crude compound was purified by flash silica gel column chromatography (30-40% ethyl acetate in pet ether) to afford [4-[(tert-butoxycarbonylamino)methyl]cyclohexen-1-yl] trifluoromethanesulfonate (2, 3.7 g, crude) as a yellow liquid. The material was used directly in the next step.

### Step 2: tert-butyl N-[[4-[[1-(2,6-dioxo-3-piperidyl)-3-methyl-2-oxo-benzimidazol-5-yl]cyclohex-3-en-1-yl]methyl]carbamate (4)

Into a 50 mL sealed tube containing a well-stirred solution of 3-[3-methyl-2-oxo-5-(4,4,5,5-tetramethyl-1,3,2-dioxaborolan-2-yl)benzimidazol-1-yl]piperidine-2,6-dione (3, 1.12 g, 2.60) mmol) and [4-[(tert-butoxy carbonylamino)methyl]cyclohexen-1-yl] trifluoromethanesulfonate (2, 11.06 mg, 2.86 mmol) in anhydrous 1,4-dioxane (10 mL) and Water (1 mL) was added sodium carbonate (825.42 mg, 7.79 mmol). The reaction mixture was degassed with nitrogen for 5 min, 1,1′-Bis(diphenylphosphino)ferrocene]dichloropalladium(II) complex with dichloromethane (317.99 mg, 389.39 μmol) was added and the tube sealed. The reaction mixture was heated to 90° C. for 2 h. The reaction mixture was diluted with water (150 mL) and extracted with ethyl acetate (2×200 mL). The combined organic layers were washed with water (200 mL). The organic layer was dried over anhydrous sodium sulfate, filtered and concentrated under reduced pressure. The residue was purified by reverse phase column chromatography (Purification method: Siliasep premium C18.25 μm, 120 g-Mobile phase: 0.1% TFA in Water/Acetonitrile) to afford tert-butyl N-[[4-[[1-(2,6-dioxo-3-piperidyl)-3-methyl-2-oxo-benzimidazol-5-yl]cyclohex-3-en-1-yl]methyl]carbamate (520 mg, 1.00 mmol, 39% yield) as an off-white solid. LCMS (ES+): m/z 369.2[M-Boc+H]⁺

### Step 3: 3-[5-[4-(aminomethyl)cyclohexen-1-yl]-3-methyl-2-oxo-benzimidazol-1-yl]piperidine-2,6-dione (5)

To a stirred solution of tert-butyl N-[[4-[[1-(2,6-dioxo-3-piperidyl)-3-methyl-2-oxo-benzimidazol-5-yl]cyclohex-3-en-1-yl]methyl]carbamate (4, 250 mg, 533.57 μmol) in DCM (1 mL) was added then cooled to (° C.) and treated with Trifluoroacetic acid (592.00 mg, 5.19 mmol, 0.4 mL). After 2 h at RT, the reaction mixture was concentrated under reduced pressure and triturated with diethyl ether (15 ml) to afford 3-[5-[4-(aminomethyl)cyclohexen-1-yl]-3-methyl-2-oxo-benzimidazol-1-yl]piperidine-2,6-dione (5, 200 mg, 352.82 μmol, 66% yield, TFA salt) as a pale yellow solid. The material was used in the next step without further purification. LCMS (ES+): m/z 369.2 [M+H]⁺

### 4-[[1-(2,6-dioxo-3-piperidyl)-3-methyl-2-oxo-benzimidazol-5-yl]amino]benzoic acid (4)

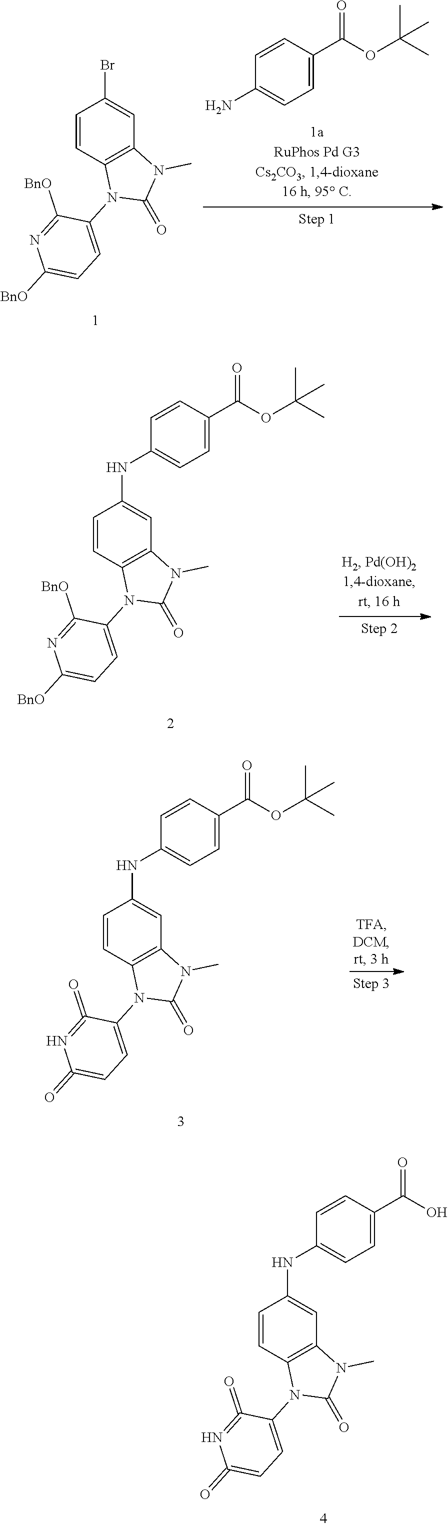

### Step 1: tert-butyl 4-[[1-(2,6-dibenzyloxy-3-pyridyl)-3-methyl-2-oxo-benzimidazol-5-yl]amino]benzoate (2)

Into a 100 mL pressure tube containing a well-stirred solution of 5-bromo-1-(2,6-dibenzyloxy-3-pyridyl)-3-methyl-benzimidazol-2-one (1, 800 mg, 1.55 mmol) and tert-butyl 4-aminobenzoate (389.19 mg, 2.01 mmol) in 1,4-dioxane (20 mL) was added sodium tert-butoxide (446.64 mg, 4.65 mmol). The reaction mixture was degassed with nitrogen gas for 5 min. RuPhos Pd G3 (194.04 mg, 232.38 μmol) was added and degassed for another 5 min. The reaction mixture was heated at 95° C. for 3 h. The reaction mixture was filtered through Celite and washed with ethyl acetate. The solvent was removed and the residue was purified by flash silica gel column chromatography (35-40% EtOAc in pet-ether) to afford tert-butyl 4-[[1-(2,6-dibenzyloxy-3-pyridyl)-3-methyl-2-oxo-benzimidazol-5-yl]amino]benzoate (2, 600 mg, 906.61 μmol, 59% yield) as a yellow solid. LCMS (ES+): m/z 629.2 [M+H]⁺

### Step 2: tert-butyl 4-[[1-(2,6-dioxo-3-piperidyl)-3-methyl-2-oxo-benzimidazol-5-yl]amino]benzoate (3)

Into a 25 mL single neck round bottom flask containing a well-stirred solution of tert-butyl 4-[[1-(2,6-dibenzyloxy-3-pyridyl)-3-methyl-2-oxo-benzimidazol-5-yl]amino]benzoate (2, 300 mg, 477.16 μmol) in 1,4-dioxane (8 mL) was added palladium hydroxide (20% on carbon) (300 mg, 1.43 mmol). The reaction mixture was stirred under hydrogen atmosphere (bladder) at RT. After 16 h the reaction mixture was filtered through Celite and washed with ethyl acetate (50 mL). The solvent was evaporated to afford tert-butyl 4-[[1-(2,6-dioxo-3-piperidyl)-3-methyl-2-oxo-benzimidazol-5-yl]amino]benzoate (3, 180 mg, 359.61 μmol, 75% yield) as a pale green solid. The material was used in the next step without purification. LCMS (ES+): m/z 451.2 [M+H]⁺

### Step 3: 4-[[1-(2,6-dioxo-3-piperidyl)-3-methyl-2-oxo-benzimidazol-5-yl]amino]benzoic acid (4)

Into a 25 mL single neck round bottom flask containing a well-stirred solution of tert-butyl 4-[[1-(2,6-dioxo-3-piperidyl)-3-methyl-2-oxo-benzimidazol-5-yl]amino]benzoate (3, 180 mg, 359.61 μmol) in DCM (2.5 mL) was added trifluoroacetic acid (820.08 mg, 7.19 mmol, 554.11 μL) at 0° C. After 2 h at RT, the solvent was removed and the residue was washed with diethyl ether and dried to afford 4-[[1-(2,6-dioxo-3-piperidyl)-3-methyl-2-oxo-benzimidazol-5-yl]amino]benzoic acid (4, 150 mg, 284.51 μmol, 79% yield, TFA salt) as a pale green solid. LCMS (ES+): m/z 395.0 [M+H]⁺

### 2-[4-[[1-(2,6-dioxo-3-piperidyl)-3-methyl-2-oxo-benzimidazol-5-yl]amino]phenyl]acetic acid (4)

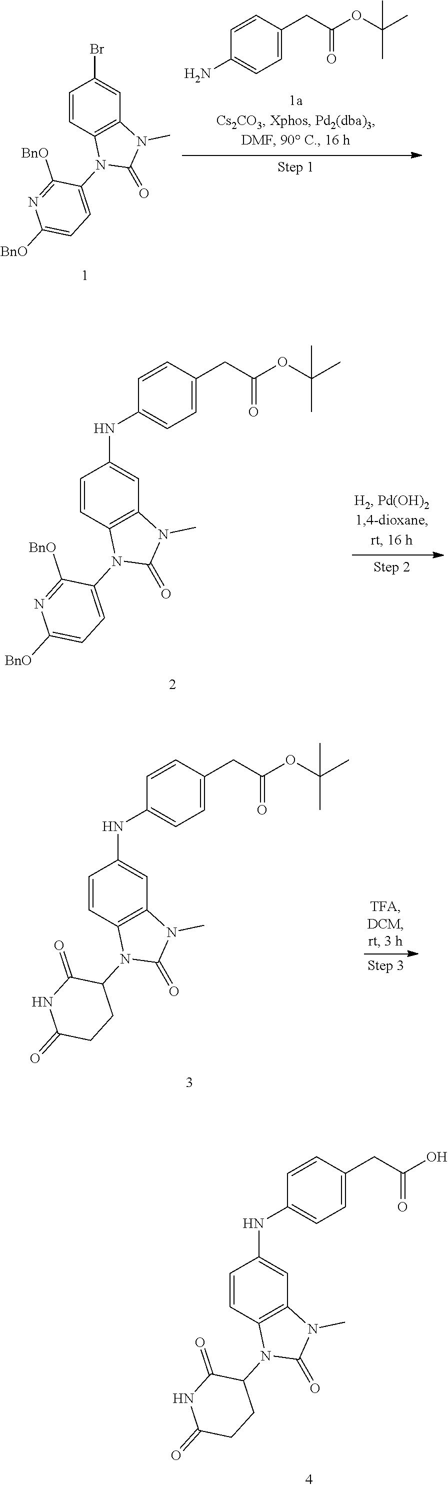 [SMILES: Cn1c(=O)n(-c2ccc(OCc3ccccc3)nc2OCc2ccccc2)c2ccc(Br)cc21 ; CC(C)(C)OC(=O)Cc1ccc(N)cc1 ; Cn1c(=O)n(-c2ccc(OCc3ccccc3)nc2OCc2ccccc2)c2ccc(Nc3ccc(CC(=O)OC(C)(C)C)cc3)cc21 ; Cn1c(=O)n(C2CCC(=O)NC2=O)c2ccc(Nc3ccc(CC(=O)OC(C)(C)C)cc3)cc21 ; Cn1c(=O)n(C2CCC(=O)NC2=O)c2ccc(Nc3ccc(CC(=O)O)cc3)cc21]

### Step 1: tert-butyl 2-[4-[[1-(2,6-dibenzyloxy-3-pyridyl)-3-methyl-2-oxo-benzimidazol-5-yl]amino]phenyl]acetate (2)

Into a 100 mL pressure tube containing a well-stirred solution of 5-bromo-1-(2,6-dibenzyloxy-3-pyridyl)-3-methyl-benzimidazol-2-one (1, 1 g, 1.92 mmol) in anhydrous 1,4-dioxane (15 mL) were added tert-butyl 2-(4-aminophenyl)acetate (1a, 1.00 g, 4.12 mmol, HCl salt) and cesium carbonate (1.88 g, 5.76 mmol). The reaction mixture was purged with nitrogen gas for 5 min. XPhos (91.48 mg, 191.89 μmol) and Pd₂(dba)₃ (351.16 mg, 383.78 μmol) were added. The reaction mixture was heated at 100° C. After 16 h, the reaction mixture was filtered through Celite and washed with ethyl acetate (80 mL). The solvent was removed and the residue purified flash silica gel column chromatography (30-40% ethyl acetate in pet ether to afford tert-butyl 2-[4-[[1-(2,6-dibenzyloxy-3-pyridyl)-3-methyl-2-oxo-benzimidazol-5-yl]amino]phenyl]acetate (2, 650 mg, 953.95 μmol, 50% yield) as a yellow solid. LCMS (ES+): m/z 643.3 [M+H]⁺

### Step 2: tert-butyl 2-[4-[[1-(2,6-dioxo-3-piperidyl)-3-methyl-2-oxo-benzimidazol-5-yl]amino]phenyl]acetate (3)

Into a 50 mL single neck round bottom flask containing a well-stirred solution of tert-butyl 2-[4-[[1-(2,6-dibenzyloxy-3-pyridyl)-3-methyl-2-oxo-benzimidazol-5-yl]amino]phenyl]acetate (2, 500 mg, 731.24 μmol) in anhydrous 1,4-dioxane (15 mL) was added palladium hydroxide (20 wt. % 50% water on carbon) (400.01 mg, 569.66 μmol). The reaction mixture was stirred under hydrogen atmosphere for 16 h at RT. The reaction mixture was filtered through Celite and washed with ethyl acetate (200 mL). The solvent was removed and the residue purified by flash silica gel column chromatography using (60-70% EtOAc in pet ether) to obtain tert-butyl 2-[4-[[1-(2,6-dioxo-3-piperidyl)-3-methyl-2-oxo-benzimidazol-5-yl]amino]phenyl]acetate (3, 300 mg, 645.84 μmol, 88% yield) as an off-white solid. LCMS (ES+): m/z 465.1 [M+H]⁺

### Step 3: 2-[4-[[1-(2,6-dioxo-3-piperidyl)-3-methyl-2-oxo-benzimidazol-5-yl]amino]phenyl]acetic acid (4)

Into a 25 mL single neck round bottom flask containing a well-stirred solution of tert-butyl 2-[4-[[1-(2,6-dioxo-3-piperidyl)-3-methyl-2-oxo-benzimidazol-5-yl]amino]phenyl]acetate (3, 300 mg, 645.84 μmol) in anhydrous DCM (8 mL) was added trifluoroacetic acid (1.47 g, 12.92 mmol, 995.11 μL) at 0° C. After 4 h at r.t., the volatiles were removed and the residue was triturated with Et₂O (2×5 mL), filtered and dried under reduced pressure to afford 2-[4-[[1-(2,6-dioxo-3-piperidyl)-3-methyl-2-oxo-benzimidazol-5-yl]amino]phenyl]acetic acid (4, 300 mg, 522.10 μmol, 81% yield, TFA salt) as an off-white solid. LCMS (ES+): m/z 409.2 [M+H]⁺

### 3-[5-[4-(aminomethyl)phenyl]-3-methyl-2-oxo-benzimidazol-1-yl]piperidine-2,6-dione (3)

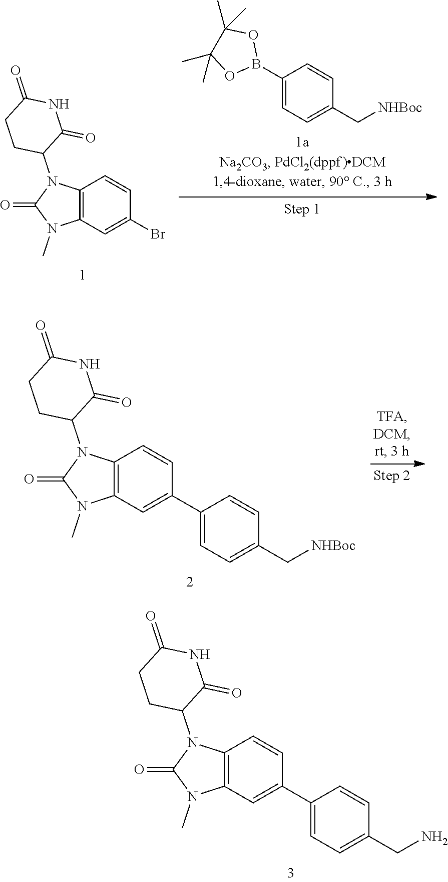 [SMILES: Cn1c(=O)n(C2CCC(=O)NC2=O)c2ccc(Br)cc21 ; CC(C)(C)OC(=O)NCc1ccc(B2OC(C)(C)C(C)(C)O2)cc1 ; Cn1c(=O)n(C2CCC(=O)NC2=O)c2ccc(-c3ccc(CNC(=O)OC(C)(C)C)cc3)cc21 ; Cn1c(=O)n(C2CCC(=O)NC2=O)c2ccc(-c3ccc(CN)cc3)cc21]

### Step 1: tert-butyl N-[[4-[[1-(2,6-dioxo-3-piperidyl)-3-methyl-2-oxo-benzimidazol-5-yl]phenyl]methyl]carbamate (2)

Into a 10 mL sealed tube containing well-stirred solution of 3-(5-bromo-3-methyl-2-oxo-benzimidazol-1-yl)piperidine-2,6-dione (1, 1 g, 2.96 mmol) and tert-butyl N-[[4-(4,4,5,5-tetramethyl-1,3,2-dioxaborolan-2-yl)phenyl]methyl]carbamate (1a, 985.43 mg, 2.96 mmol) in 1,4-dioxane (6 mL) and water (1 mL) was added sodium carbonate (313.43 mg, 2.96 mmol). The mixture was purged with nitrogen gas for 15 min. Then Pd(dppf)Cl₂·DCM (241.50 mg, 295.72 μmol) was added and the reaction mixture was heated at 90° C. After 3 h the reaction mixture was diluted with ethyl acetate (100 mL), washed with water (2×30 mL), dried over anhydrous Na₂SO₄, filtered and concentrated under reduced pressure. The residue was purified by flash silica gel column chromatography (90% EtOAc in pet ether) to afford tert-butyl N-[[4-[[1-(2,6-dioxo-3-piperidyl)-3-methyl-2-oxo-benzimidazol-5-yl]phenyl]methyl]carbamate (2, 450 mg, 781.40 μmol, 26% yield) as a light yellow solid. LCMS (ES+): m/z 465.0 [M+H]⁺

### Step 2: 3-[5-[4-(aminomethyl)phenyl]-3-methyl-2-oxo-benzimidazol-1-yl]piperidine-2,6-dione (3)

Into a 50 mL single neck round bottom flask containing well-stirred solution of tert-butyl N-[[4-[1-(2,6-dioxo-3-piperidyl)-3-methyl-2-oxo-benzimidazol-5-yl]phenyl]methyl]carbamate (2, 450 mg, 775.01 μmol) in anhydrous DCM (8 mL) was added TFA (2.22 g, 19.47 mmol, 1.5 mL). After 3 h, the volatiles were removed and the residue was triturated with diethyl ether (100 mL), filtered and dried to afford 3-[5-[4-(aminomethyl)phenyl]-3-methyl-2-oxo-benzimidazol-1-yl]piperidine-2,6-dione (3, 350 mg, 634.13 μmol, 82% yield, TFA salt) as an off-white solid. LCMS (ES+): m/z 365.2 [M+H]⁺

### 3-[3-methyl-2-oxo-5-[4-(4-piperidyl)-1-piperidyl]benzimidazol-1-yl]piperidine-2,6-dione (5)

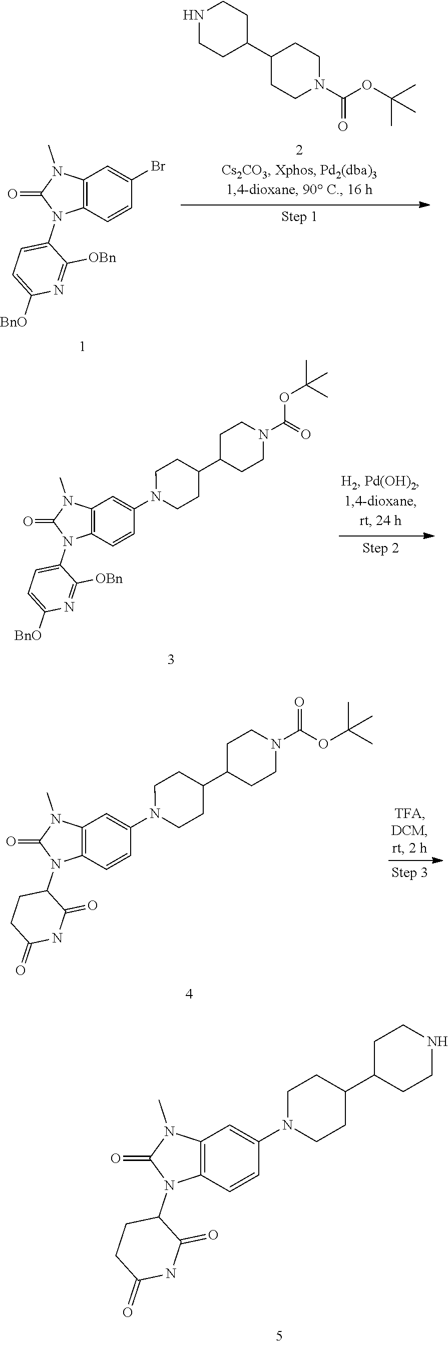 [SMILES: CC(C)(C)OC(=O)N1CCC(C2CCNCC2)CC1 ; Cn1c(=O)n(-c2ccc(OCc3ccccc3)nc2OCc2ccccc2)c2ccc(Br)cc21 ; Cn1c(=O)n(-c2ccc(OCc3ccccc3)nc2OCc2ccccc2)c2ccc(N3CCC(C4CCN(C(=O)OC(C)(C)C)CC4)CC3)cc21 ; Cn1c(=O)n(C2CCC(=O)NC2=O)c2ccc(N3CCC(C4CCN(C(=O)OC(C)(C)C)CC4)CC3)cc21 ; Cn1c(=O)n(C2CCC(=O)NC2=O)c2ccc(N3CCC(C4CCNCC4)CC3)cc21]

### Step 1: tert-butyl 4-[1-[1-(2,6-dibenzyloxy-3-pyridyl)-3-methyl-2-oxo-benzimidazol-5-yl]-4-piperidyl]piperidine-1-carboxylate (3)

Into a 10 mL pressure tube containing a well-stirred solution of 5-bromo-1-(2,6-dibenzyloxy-3-pyridyl)-3-methyl-benzimidazol-2-one (1, 750 mg, 1.44 mmol) and tert-butyl 4-(4-piperidyl)piperidine-1-carboxylate (2, 578.88 mg, 2.16 mmol) in anhydrous 1,4-dioxane (5 mL) was added cesium carbonate (1.41 g, 4.31 mmol) and the mixture was purged with nitrogen gas for 15 min. Then tris(dibenzylideneacetone)dipalladium(0)) (131.67 mg, 143.79 μmol) and XPhos (68.55 mg, 143.79 μmol) were added. After 16 h at 90° C., the reaction mixture was filtered through Celite. The filtrate was concentrated under reduced pressure and purified by flash silica gel (230)-400 mesh) column chromatography (50% EtOAc in Pet-ether) to afford tert-butyl 4-[1-[1-(2,6-dibenzyloxy-3-pyridyl)-3-methyl-2-oxo-benzimidazol-5-yl]-4-piperidyl]piperidine-1-carboxylate (3, 710 mg, 1.01 mmol, 70% yield) as light brown solid. LCMS (ES+): m/z 704.3 [M+H]⁺

### Step 2: tert-butyl 4-[1-[1-(2,6-dioxo-3-piperidyl)-3-methyl-2-oxo-benzimidazol-5-yl]-4-piperidyl]piperidine-1-carboxylate (4)

Into a 100 mL single neck round bottom flask containing a well-stirred solution of tert-butyl 4-[1-[1-(2,6-dibenzyloxy-3-pyridyl)-3-methyl-2-oxo-benzimidazol-5-yl]-4-piperidyl]piperidine-1-carboxylate (3, 710 mg, 998.63 μmol) in anhydrous 1,4-dioxane (10 mL) was added palladium hydroxide (20 wt. %, 50% water on carbon) (500 mg, 712.07 μmol, 20% purity). The suspension was stirred at RT under hydrogen atmosphere. After 24 h, the reaction mixture was filtered through Celite and washed with 1,4-dioxane (200 mL). The filtrate was concentrated under reduced pressure to afford tert-butyl 4-[1-[1-(2,6-dioxo-3-piperidyl)-3-methyl-2-oxo-benzimidazol-5-yl]-4-piperidyl]piperidine-1-carboxylate (4, 450 mg, 831.28 μmol, 83% yield) as an off-white solid. The material was used in the next step without purification. LCMS (ES+): m/z 526.2 [M+H]⁺

### Step 3: 3-[3-methyl-2-oxo-5-[4-(4-piperidyl)-1-piperidyl]benzimidazol-1-yl]piperidine-2,6-dione (5)

Into a 100 mL single neck round bottom flask containing well-stirred solution of tert-butyl 4-[1-[1-(2,6-dioxo-3-piperidyl)-3-methyl-2-oxo-benzimidazol-5-yl]-4-piperidyl]piperidine-1-carboxylate (4, 450 mg, 831.28 μmol) in anhydrous DCM (8 mL) was added TFA (2.96 g, 25.96 mmol, 2 mL). After 2 h the solvent was removed and the residue triturated with diethyl ether (100 mL) to afford 3-[3-methyl-2-oxo-5-[4-(4-piperidyl)-1-piperidyl]benzimidazol-1-yl]piperidine-2,6-dione (5, 450 mg, 619.77 μmol, 75% yield, TFA salt) as an off-white solid. LCMS (ES+): m/z 426.3 [M+H]⁺

### 2-[4-[4-[(2,6-dioxo-3-piperidyl)amino]phenyl]-3,3-difluoro-1-piperidyl]acetic acid (4)

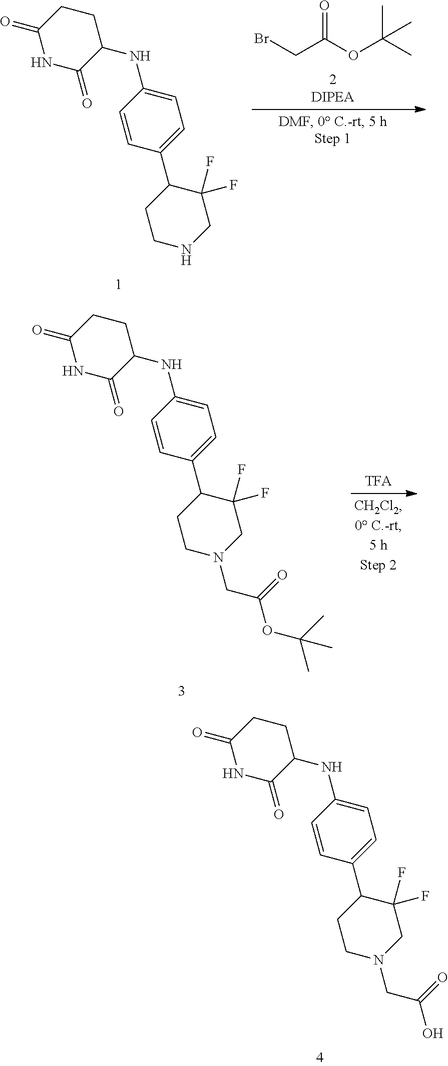

### Step 1: tert-butyl 2-[4-[4-[(2,6-dioxo-3-piperidyl)amino]phenyl]-3,3-difluoro-1-piperidyl]acetate (3)

Into a 25 mL single neck round bottom flask containing a well-stirred solution of 3-[4-(3,3-difluoro-4-piperidyl)anilino]piperidine-2,6-dione (1, 400 mg, 1.11 mmol, HCl salt) in anhydrous DMF (5 mL) at 0° C. under nitrogen atmosphere were added DIPEA (431.05 mg, 3.34 mmol, 580.93 L) and tert-butyl bromoacetate (2, 238.53 mg, 1.22 mmol, 179.35 μL). After 5 h at RT, the reaction mixture was poured into water and extracted with DCM (3×10 mL). The organic layers were combined, washed with water (15 mL), brine (15 mL), dried over anhydrous sodium sulfate and concentrated under reduced pressure. The residue was triturated with Et₂O to obtain tert-butyl 2-[4-[4-[(2,6-dioxo-3-piperidyl)amino]phenyl]-3,3-difluoro-1-piperidyl]acetate (3, 445 mg, 915.47 μmol, 82% yield) as a green solid. LCMS (ES+): m/z 438.2 [M+H]⁺

### Step 2: 2-[4-[4-[(2,6-dioxo-3-piperidyl)amino]phenyl]-3,3-difluoro-1-piperidyl]acetic acid (4)

Into a 25 mL single neck round bottom flask containing a well-stirred solution of tert-butyl 2-[4-[4-[(2,6-dioxo-3-piperidyl)amino]phenyl]-3,3-difluoro-1-piperidyl]acetate (3, 440 mg, 905.19 μmol) in anhydrous DCM (5 mL) was added TFA (2.58 g, 22.63 mmol, 1.74 mL) at 0° C. After 5 h at RT, the reaction mixture was concentrated under reduced pressure and triturated with diethyl ether (5 mL) to afford 2-[4-[4-[(2,6-dioxo-3-piperidyl)amino]phenyl]-3,3-difluoro-1-piperidyl]acetic acid (4, 440 mg, 822.45 μmol, 91% yield, TFA salt) as a dark-green solid. LCMS (ES+): m/z 382.0 [M+H]⁺

### 2-(4-(3-(2,6-dioxopiperidin-3-yl)-1-methyl-1H-indazol-6-yl)-3,3-difluoropiperidin-1-yl)acetic acid (5)

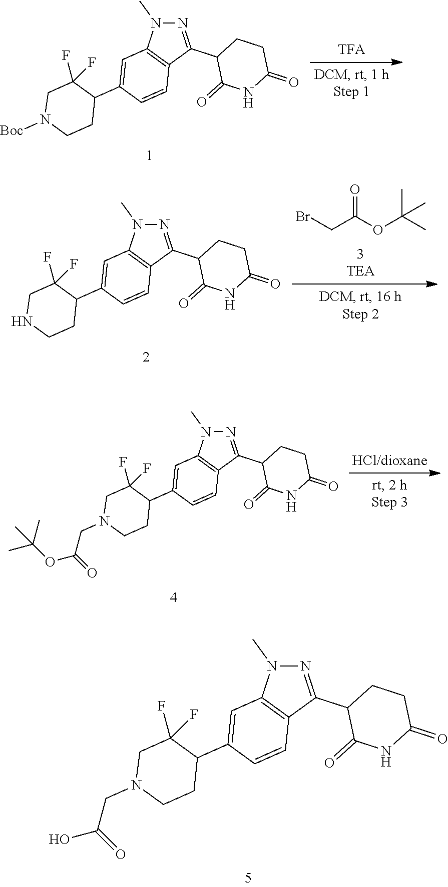

### Step 1: 3-(6-(3,3-difluoropiperidin-4-yl)-1-methyl-1H-indazol-3-yl)piperidine-2,6-dione (2)

To a solution tert-butyl tert-butyl 4-(3-(2,6-dioxopiperidin-3-yl)-1-methyl-1H-indazol-6-yl)-3,3-difluoropiperidine-1-carboxylate (1, 1 g, 2.16 mmol) in DCM (10 mL) was added TFA (15.40 g, 135.06 mmol, 10.00 mL). The reaction mixture was stirred at 20° C. for 1 h. The reaction mixture was concentrated under reduced pressure to afford 3-(6-(3,3-difluoropiperidin-4-yl)-1-methyl-1H-indazol-3-yl)piperidine-2,6-dione (2, 1.2 g, 2.03 mmol, 94% yield, 2 TFA salt) as a yellow oil. The material was used in the next step without further purification. LCMS (ESI): m/z 363.0 [M+H]⁺

### Step 2: tert-butyl 2-(4-(3-(2,6-dioxopiperidin-3-yl)-1-methyl-1H-indazol-6-yl)-3,3-difluoropiperidin-1-yl)acetate (4)

To a solution of 3-(6-(3,3-difluoropiperidin-4-yl)-1-methyl-1H-indazol-3-yl)piperidine-2,6-dione (2, 1.2 g, 3.31 mmol) in DMF (20 mL) was added Et₃N (1.68 g, 16.56 mmol, 2.30 mL) and tert-butyl 2-bromoacetate (3, 1.29 g, 6.62 mmol, 978.67 μL). The reaction mixture was stirred at 20° C. for 16 h. The reaction mixture was diluted with water (20 mL) and extracted with EtOAc (20 mL×3). The combined organic layer was washed with brine (20 mL) and concentrated under reduced pressure to afford tert-butyl 2-(4-(3-(2,6-dioxopiperidin-3-yl)-1-methyl-1H-indazol-6-yl)-3,3-difluoropiperidin-1-yl)acetate (4, 1.2 g, 2.52 mmol, 76% yield) as white solid. The material was used in the next step without purification. LCMS (ESI): m/z 477.3 [M+H]⁺

### Step 3: 2-(4-(3-(2,6-dioxopiperidin-3-yl)-1-methyl-1H-indazol-6-yl)-3,3-difluoropiperidin-1-yl)acetic acid (5)

A solution of tert-butyl 2-(4-(3-(2,6-dioxopiperidin-3-yl)-1-methyl-1H-indazol-6-yl)-3,3-difluoropiperidin-1-yl)acetate (4, 950 mg, 1.99 mmol) in HCl/dioxane (4 M, 10 mL) was stirred at 20° C. for 2 h. The reaction mixture was concentrated under reduced pressure to afford 2-(4-(3-(2,6-dioxopiperidin-3-yl)-1-methyl-1H-indazol-6-yl)-3,3-difluoropiperidin-1-yl)acetic acid (5, 900 mg, 1.85 mmol, 92% yield, HCl salt) as white solid. The material was used in the next step without purification. LCMS (ESI): m/z 421.0 [M+H]⁺

### 3-(4-(1-(2,6-dioxopiperidin-3-yl)-3-methyl-2-oxo-2,3-dihydro-1H-benzo[d]imidazol-5-yl)phenyl)propanoic acid (8)

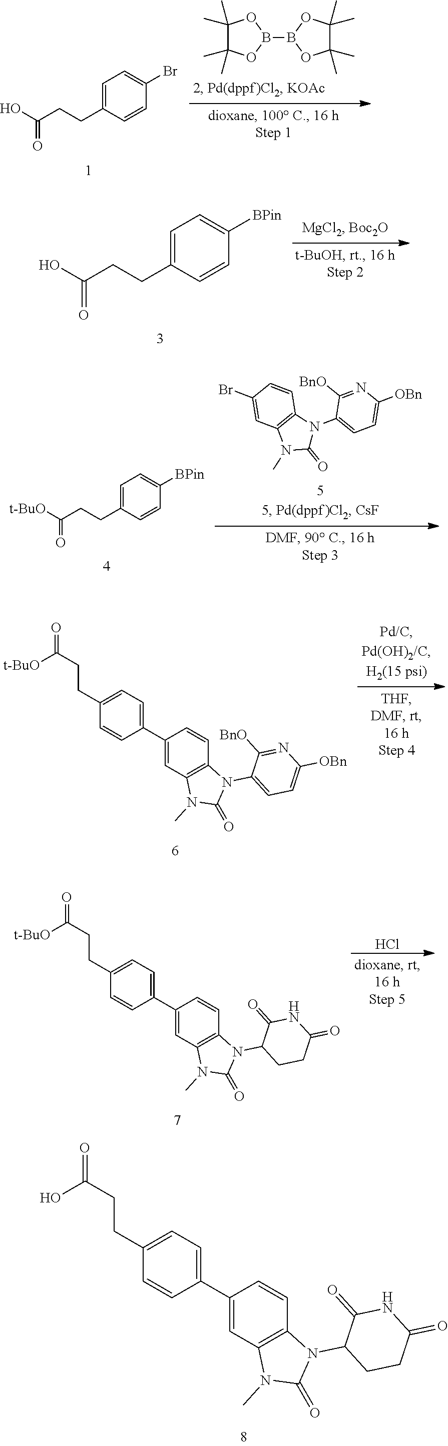

### Step 1: 3-(4-(4,4,5,5-tetramethyl-1,3,2-dioxaborolan-2-yl)phenyl)propanoic acid (3)

To a solution of 3-(4-bromophenyl)propanoic acid (1, 10 g, 43.65 mmol) and 4,4,5,5-tetramethyl-2-(4,4,5,5-tetramethyl-1,3,2-dioxaborolan-2-yl)-1,3,2-dioxaborolane (2, 16.63 g, 65.48 mmol) in dioxane (100 mL) was added KOAc (21.42 g, 218.27 mmol, 13.64 mL) under N₂ atmosphere. Subsequently, Pd(dppf)Cl₂ (1.60 g, 2.18 mmol, 0.05 eq) was added. The reaction mixture was stirred at 100° C. for 16 h. The reaction mixture was poured into water (20 mL) and extracted with ethyl acetate (2×20 mL). The combined organic layers were washed with brine (30 mL), dried over anhydrous Na₂SO₄, filtered and concentrated. The residue was purified by flash silica gel chromatography (SiO₂, petroleum ether/ethyl acetate=1/0 to 4/1) to afford 3-(4-(4,4,5,5-tetramethyl-1,3,2-dioxaborolan-2-yl)phenyl)propanoic acid (3, 10 g, 36.21 mmol, 83% yield) as a yellow solid. The material was used in the next step without further characterization.

### Step 2: tert-butyl 3-(4-(4,4,5,5-tetramethyl-1,3,2-dioxaborolan-2-yl)phenyl)propanoate (4)

A mixture of 3-(4-(4,4,5,5-tetramethyl-1,3,2-dioxaborolan-2-yl)phenyl)propanoic acid (3, 5 g, 18.11 mmol), Boc₂O (5.14 g, 23.54 mmol, 5.40 mL) and MgCl₂ (172.40 mg, 1.81 mmol) in t-BuOH (50 mL) was stirred at 20° C. for 16 h. The reaction mixture was poured into water (70 mL) and extracted with ethyl acetate (2×70 mL). The combined organic phase was washed with brine (70 mL), dried by anhydrous Na₂SO₄, filtered and concentrated. The residue was purified by flash silica gel chromatography (SiO₂, petroleum ether/ethyl acetate=1/0) to 10/1) to afford tert-butyl 3-(4-(4,4,5,5-tetramethyl-1,3,2-dioxaborolan-2-yl)phenyl)propanoate (4, 2 g, 12.64 mmol, 70% yield) as colorless oil.

1H NMR (400 MHZ, chloroform-d) δ=7.75 (d, J=7.6 Hz, 2H), 7.23 (d, J=8.0 Hz, 2H), 2.94 (t, J=7.6 Hz, 2H), 2.56 (t, J=8.0 Hz, 2H), 1.44 (s, 9H), 1.36 (s, 12H)

### Step 3: tert-butyl 3-(4-(1-(2,6-bis(benzyloxy)pyridin-3-yl)-3-methyl-2-oxo-2,3-dihydro-1H-benzo[d]imidazol-5-yl)phenyl)propanoate (6)

A mixture of tert-butyl 3-(4-(4,4,5,5-tetramethyl-1,3,2-dioxaborolan-2-yl)phenyl)propanoate (4, 3.7 g, 11.14 mmol), 1-(2,6-bis(benzyloxy)pyridin-3-yl)-5-bromo-3-methyl-1H-benzo[d]imidazol-2(3H)-one (5, 4.42 g, 8.57 mmol). Pd(dppf)Cl₂ (313.41 mg, 428.33 μmol) and CsF (3.90 g, 25.70 mmol, 947.54 μL) in DMF (30 mL) was degassed and purged with N₂ for 3 times. The mixture was stirred at 90° C. for 16 h under N₂ atmosphere. The reaction mixture was filtered and poured into water (70 mL) and extracted with ethyl acetate (2×70 mL). The combined organic phase was washed with brine (70 mL), dried over anhydrous Na₂SO₄, filtered and concentrated in vacuum. The residue was purified by flash silica gel chromatography (SiO₂, petroleum ether/ethyl acetate=1/0 to 4/1) to afford tert-butyl 3-(4-(1-(2,6-bis(benzyloxy)pyridin-3-yl)-3-methyl-2-oxo-2,3-dihydro-1H-benzo[d]imidazol-5-yl)phenyl)propanoate (6, 5 g, 7.71 mmol, 90% yield) as white solid. LCMS (ESI): m/z 642.3 [M+H]⁺

### Step 4: tert-butyl 3-(4-(1-(2,6-dioxopiperidin-3-yl)-3-methyl-2-oxo-2,3-dihydro-1H-benzo[d]imidazol-5-yl)phenyl)propanoate (7)

To a solution of tert-butyl 3-(4-(1-(2,6-bis(benzyloxy)pyridin-3-yl)-3-methyl-2-oxo-2,3-dihydro-1H-benzo[d]imidazol-5-yl)phenyl)propanoate (6, 5 g, 7.71 mmol) in THF (250 mL) and DMF (50) mL) were added Pd/C (20 mg) and Pd(OH)₂/C (20 mg) under N₂ atmosphere. The suspension was degassed and purged with H₂ for 3 times. The mixture was stirred under H₂ (15 psi) at 20° C. for 16 h. The reaction mixture was filtered and concentrated in vacuum. The residue was purified by reversed phase flash chromatography (flow: 100 mL/min; gradient: from 5-65% MeCN in water (0.1% TFA) over 33 min; column: Welch Ultimate XB-C18, 20-40 μm, 120 Å, I.D. 57 mm×H 235 mm) to afford tert-butyl 3-(4-(1-(2,6-dioxopiperidin-3-yl)-3-methyl-2-oxo-2,3-dihydro-1H-benzo[d]imidazol-5-yl)phenyl)propanoate (7, 2.9 g, 6.26 mmol, 81% yield) as a white solid. LCMS (ESI): m/z 464.2 [M+H]⁺

¹H NMR (400 MHZ, d₆-DMSO) δ 11.12 (s, 1H), 7.61 (d, J=8.0 Hz, 2H), 7.48 (d, J=1.2 Hz, 1H), 7.36-7.27 (m, 3H), 7.18 (d, J=8.4 Hz, 1H), 5.40 (dd, J=5.6, 12.8 Hz, 1H), 3.41 (s, 3H), 2.98-2.89 (m, 1H), 2.85 (t, J=8.0 Hz, 2H), 2.80-2.62 (m, 2H), 2.57-2.53 (m, 2H), 2.11-2.00 (m, 1H), 1.38 (s, 9H)

### Step 5: 3-(4-(1-(2,6-dioxopiperidin-3-yl)-3-methyl-2-oxo-2,3-dihydro-1H-benzo[d]imidazol-5-yl)phenyl)propanoic acid (8)

To a solution of tert-butyl 3-(4-(1-(2,6-dioxopiperidin-3-yl)-3-methyl-2-oxo-2,3-dihydro-1H-benzo[d]imidazol-5-yl)phenyl)propanoate (7, 500 mg, 1.08 mmol) in 1,4-dioxane (5 mL) was added HCl/dioxane (4 M, 5 mL). The mixture was stirred at 20° C. for 16 h. The reaction mixture was concentrated in vacuum to afford 3-(4-(1-(2,6-dioxopiperidin-3-yl)-3-methyl-2-oxo-2,3-dihydro-1H-benzo[d]imidazol-5-yl)phenyl)propanoic acid (8, 450 mg, 1.01 mmol, 94% yield, HCl salt). The material was used in the next step without further purification. LCMS (ESI): m/z 408.1 [M+H]⁺

### 3-(4-(3-(2,6-dioxopiperidin-3-yl)-1-methyl-1H-indazol-6-yl)-3,3-difluoropiperidin-1-yl)cyclobutanecarboxylic acid (4)

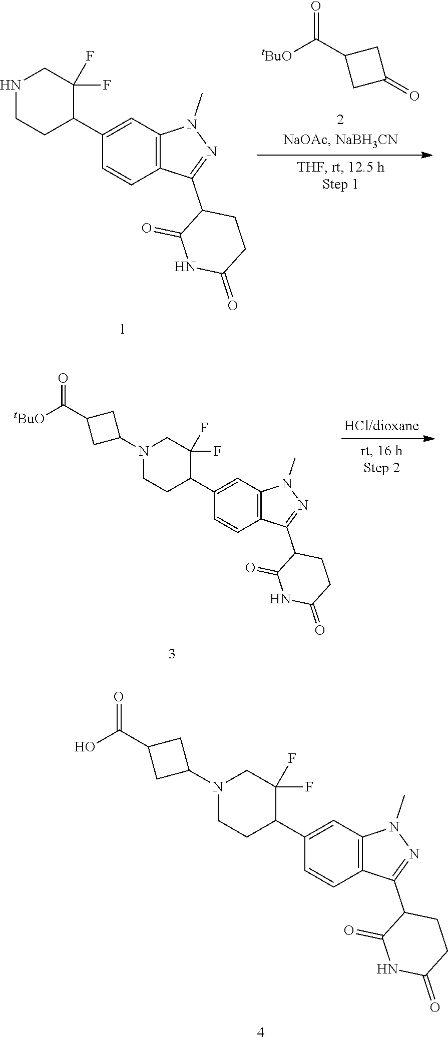 [SMILES: Cn1nc(C2CCC(=O)NC2=O)c2ccc(C3CCNCC3(F)F)cc21 ; CC(C)(C)OC(=O)C1CC(=O)C1 ; Cn1nc(C2CCC(=O)NC2=O)c2ccc(C3CCN(C4CC(C(=O)OC(C)(C)C)C4)CC3(F)F)cc21 ; Cn1nc(C2CCC(=O)NC2=O)c2ccc(C3CCN(C4CC(C(=O)O)C4)CC3(F)F)cc21]

### Step 1: tert-butyl 3-(4-(3-(2,6-dioxopiperidin-3-yl)-1-methyl-1H-indazol-6-yl)-3,3-difluoropiperidin-1-yl)cyclobutanecarboxylate (3)

To a solution of 3-(6-(3,3-difluoropiperidin-4-yl)-1-methyl-1H-indazol-3-yl)piperidine-2,6-dione (1, 300 mg, 752.19 μmol, HCl salt) and tert-butyl 3-oxocyclobutanecarboxylate (2, 153.63 mg, 902.63 μmol) in THF (5 mL) was added NaOAc (123.41 mg, 1.50 mmol) at 15° C. and stirred for 0.5 h. Then NaBH₃CN (236.35 mg, 3.76 mmol) was added. The mixture was stirred at 15° C. for 12 h. The reaction mixture was quenched by addition H₂O (20 mL) at 0° C. The resulting mixture was extracted with ethyl acetate (2×15 mL). The combined organic layers were washed with brine (20 mL), dried over Na₂SO₄, filtered and concentrated under reduced pressure. The residue was purified by flash silica gel chromatography (ISCOR: 10 g SepaFlash R Silica Flash Column, Flow: 36 mL/min, Eluent of 0-40% ethyl acetate/petroleum ether) to afford tert-butyl 3-(4-(3-(2,6-dioxopiperidin-3-yl)-1-methyl-1H-indazol-6-yl)-3,3-difluoropiperidin-1-yl)cyclobutanecarboxylate (3, 185 mg, 347.38 μmol, 46% yield) as a white solid. LCMS (ESI): m/z 517.2 [M+H]⁺

### Step 2: 3-(4-(3-(2,6-dioxopiperidin-3-yl)-1-methyl-1H-indazol-6-yl)-3,3-difluoropiperidin-1-yl)cyclobutanecarboxylic acid (4)

A solution of tert-butyl 3-(4-(3-(2,6-dioxopiperidin-3-yl)-1-methyl-1H-indazol-6-yl)-3,3-difluoropiperidin-1-yl)cyclobutanecarboxylate (3, 185 mg, 358.13 μmol) in HCl/1,4-dioxane (4 M, 10 mL) was stirred at 15° C. for 16 h. The reaction mixture was concentrated to afford 3-(4-(3-(2,6-dioxopiperidin-3-yl)-1-methyl-1H-indazol-6-yl)-3,3-difluoropiperidin-1-yl)cyclobutanecarboxylic acid (4, 195 mg, 365.58 μmol, 100% yield, HCl salt). The material was used in the next step without further purification. LCMS (ESI): m/z 461.4 [M+H]⁺

### 2-(4-(1-(2,6-bis(benzyloxy)pyridin-3-yl)-3-methyl-2-oxo-2,3-dihydro-1H-benzo[d]imidazol-5-yl)-3-fluorophenyl)acetic acid (6)

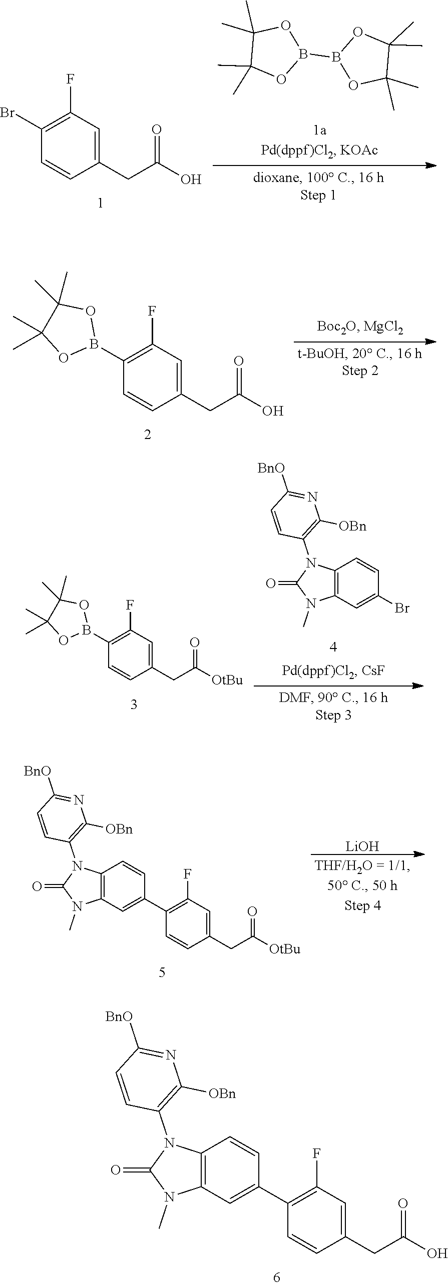

### Step 1: 2-(3-fluoro-4-(4,4,5,5-tetramethyl-1,3,2-dioxaborolan-2-yl)phenyl)acetic acid (2)

To a solution of 2-(4-bromo-3-fluoro-phenyl)acetic acid (1, 2 g, 8.58 mmol) and Bis(pinacolato)diboron (1a, 3.27 g, 12.87 mmol) in dioxane (5 mL) was added KOAc (4.21 g, 42.91 mmol) under N₂ atmosphere. Subsequently, Pd(dppf)Cl₂ (313.99 mg, 429.12 μmol, 0.05 eq) was added to the reaction mixture and reaction mixture was stirred at 100° C. for 16 h. The reaction mixture diluted with water (10 mL) and extracted with ethyl acetate (20 mL×3). The organic phases were combined and washed with brine (30 mL), dried over anhydrous Na₂SO₄, filtered and concentrated under reduced pressure. The residue was purified by column chromatography (SiO₂, petroleum ether/ethyl acetate=100/0 to 1/1) to afford 2-(3-fluoro-4-(4,4,5,5-tetramethyl-1,3,2-dioxaborolan-2-yl)phenyl)acetic acid (2, 1.7 g, 6.07 mmol, 71% yield).

¹H NMR (400 MHZ, CDCl₃) δ 7.71 (dd, J=6.4, 7.2 Hz, 1H), 7.07 (dd, J=1.2, 7.6 Hz, 1H), 7.01-6.97 (m, 1H), 3.65 (s, 2H), 1.36 (s, 12H).

### Step 2: tert-butyl 2-(3-fluoro-4-(4,4,5,5-tetramethyl-1,3,2-dioxaborolan-2-yl)phenyl)acetate (3)

A mixture of 2-(3-fluoro-4-(4,4,5,5-tetramethyl-1,3,2-dioxaborolan-2-yl)phenyl)acetic acid (2, 1.7 g, 6.07 mmol) and Boc₂O (1.72 g, 7.89 mmol, 1.81 mL) in tert-butyl alcohol (10 mL) was added MgCl₂ (57.79 mg, 606.93 μmol) and stirred for 16 h. The reaction mixture was diluted with water (10 mL) and extracted with ethyl acetate (20 mL×3). The organic phases were combined and washed with brine (30 mL), dried over anhydrous Na₂SO₄, filtered and concentrated under reduced pressure. The residue was purified by column chromatography (SiO₂, petroleum ether/ethyl acetate=100/0) to 5/1) to afford tert-butyl 2-(3-fluoro-4-(4,4,5,5-tetramethyl-1,3,2-dioxaborolan-2-yl)phenyl)acetate (3, 1.3 g, 3.87 mmol, 64% yield).

¹H NMR (400 MHZ, CDCl₃) δ 7.67 (dd, J=6.4, 7.2 Hz, 1H), 7.04 (dd, J=1.2, 7.6 Hz, 1H), 6.97 (dd, J=1.2, 10.0 Hz, 1H), 3.51 (s, 2H), 1.42 (s, 9H), 1.35 (s, 12H).

### Step 3: tert-butyl 2-(4-(1-(2,6-bis(benzyloxy)pyridin-3-yl)-3-methyl-2-oxo-2,3-dihydro-1H-benzo[d]imidazol-5-yl)-3-fluorophenyl)acetate (5)

A mixture of tert-butyl 2-(3-fluoro-4-(4,4,5,5-tetramethyl-1,3,2-dioxaborolan-2-yl)phenyl)acetate (3, 1.2 g, 3.57 mmol) and 1-(2,6-bis(benzyloxy)pyridin-3-yl)-5-bromo-3-methyl-1H-benzo[d]imidazol-2(3H)-one (4, 1.42 g, 2.75 mmol) in DMF (10 mL) were added CsF (1.25 g, 8.24 mmol) and Pd(dppf)Cl₂ (100.45 mg, 137.28 μmol), and stirred at 90° C. for 16 h under N₂. The reaction mixture was filtered and concentrated under reduced pressure. The residue was purified by flash silica gel chromatography (SiO₂, petroleum ether/ethylacetate=100/1˜3/2) to afford tert-butyl 2-(4-(1-(2,6-bis(benzyloxy)pyridin-3-yl)-3-methyl-2-oxo-2,3-dihydro-1H-benzo[d]imidazol-5-yl)-3-fluorophenyl)acetate (5, 1.5 g, 2.11 mmol, 77% yield). LCMS (ESI): m/z 646.2 [M+H]⁺

¹H NMR (400 MHZ, d6-DMSO) δ 7.86-7.83 (m, 1H), 7.51-7.44 (m, 3H), 7.40-7.36 (m, 3H), 7.29-7.25 (m, 6H), 7.20-7.17 (m, 2H), 6.78 (d, J=8.2 Hz, 1H), 6.65-6.61 (m, 1H), 5.42-5.37 (m, 4H), 3.93 (s, 1H), 3.65 (s, 2H), 3.43 (s, 3H), 1.43 (s, 9H).

### Step 4: 2-(4-(1-(2,6-bis(benzyloxy)pyridin-3-yl)-3-methyl-2-oxo-2,3-dihydro-1H-benzo[d]imidazol-5-yl)-3-fluorophenyl)acetic acid (6)

To a solution of tert-butyl 2-(4-(1-(2,6-bis(benzyloxy)pyridin-3-yl)-3-methyl-2-oxo-2,3-dihydro-1H-benzo[d]imidazol-5-yl)-3-fluorophenyl)acetate (5, 201.24 mg, 311.65 μmol) in THF (5 mL) and H₂O (5 mL) was added LiOH·H₂O (200 mg, 4.77 mmol). The mixture was stirred at 50° C. for 32 h. Then LiOH·H₂O (200 mg, 4.77 mmol) was added at 25° C. and the mixture was stirred at 50° C. for 4 h. The mixture was adjusted pH to 3˜4 with IN HCl at 10-20° C. The mixture was extracted with ethyl acetate (20 mL×2). The combined organic layers were washed with brine (20 mL×2), dried over Na₂SO₄, filtered and concentrated under reduced pressure to afford 2-(4-(1-(2,6-bis(benzyloxy)pyridin-3-yl)-3-methyl-2-oxo-2,3-dihydro-1H-benzo[d]imidazol-5-yl)-3-fluorophenyl)acetic acid (6, 180 mg, 305.29 μmol, 98% yield). LCMS (ESI): m/z 590.3 [M+H]⁺

### 2-[4-[1-(2,6-dioxo-3-piperidyl)-3-methyl-2-oxo-benzimidazol-5-yl]-1-piperidyl]acetic acid (3)

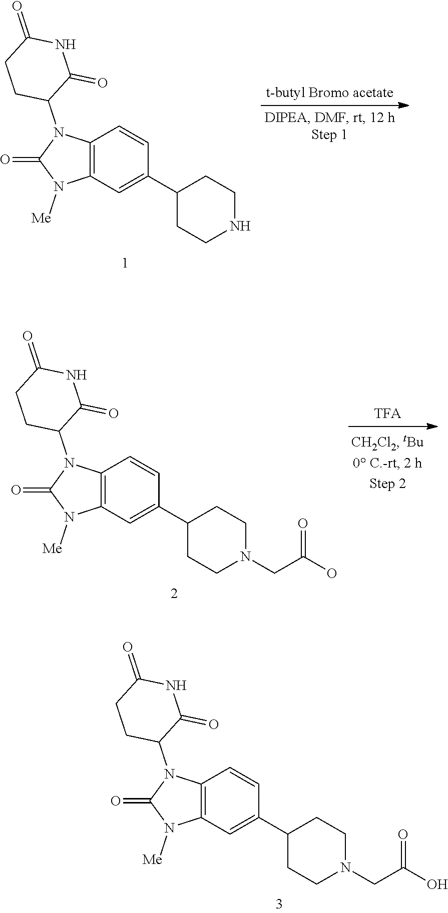 [SMILES: Cn1c(=O)n(C2CCC(=O)NC2=O)c2ccc(C3CCNCC3)cc21 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)O)CC3)cc21]

### Step 1: tert-butyl 2-[4-[1-(2,6-dioxo-3-piperidyl)-3-methyl-2-oxo-benzimidazol-5-yl]-1-piperidyl]acetate (2)

Into a 50 mL single neck, round bottom flask containing a well-stirred solution of 3-[3-methyl-2-oxo-5-(4-piperidyl)benzimidazol-1-yl]piperidine-2,6-dione (1, 1 g, 2.92 mmol) in dry DMF (7 mL) were added tert-butyl 2-bromoacetate (626.65 mg, 3.21 mmol, 471.17 μL) and DIPEA (1.13 g, 8.76 mmol, 1.53 mL) under nitrogen atmosphere. After 16 h, the reaction mixture was poured into ice-cold water (20 mL) and the precipitate was filtered and dried under vacuum to afford tert-butyl 2-[4-[1-(2,6-dioxo-3-piperidyl)-3-methyl-2-oxo-benzimidazol-5-yl]-1-piperidyl]acetate (2, 1 g, 2.10 mmol, 72% yield) as an off-white solid. LCMS (ES+): m/z 457.4 [M+H]⁺

### Step 2: 2-[4-[1-(2,6-dioxo-3-piperidyl)-3-methyl-2-oxo-benzimidazol-5-yl]-1-piperidyl]acetic acid (3)

Into a 50 mL single neck round bottom flask containing a well-stirred solution of tert-butyl 2-[4-[1-(2,6-dioxo-3-piperidyl)-3-methyl-2-oxo-benzimidazol-5-yl]-1-piperidyl]acetate (2, 1 g, 2.19 mmol) in dry DCM (10 mL) was added TFA (1.25 g, 10.95 mmol, 843.78 μL) dropwise under nitrogen atmosphere at 0° C. The reaction mixture was stirred for 2 h at ambient temperature. The reaction mixture was concentrated under reduced pressure and the residue was co-distilled with toluene (2×15 mL) and triturated with diethyl ether (20 mL) to afford 2-[4-[1-(2,6-dioxo-3-piperidyl)-3-methyl-2-oxo-benzimidazol-5-yl]-1-piperidyl]acetic acid (3, 1.1 g, 1.92 mmol, 88% yield, TFA salt) as an off-white solid. LCMS (ES+): m/z 401.2 [M+H]⁺

### 3-[5-(4-aminoanilino)-3-methyl-2-oxo-benzimidazol-1-yl]piperidine-2,6-dione (4)

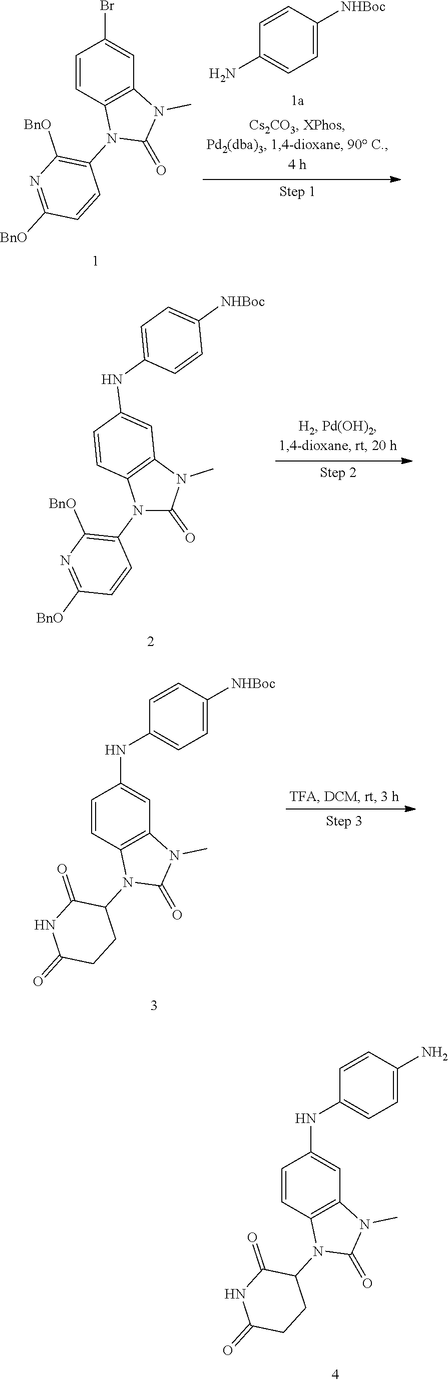 [SMILES: Cn1c(=O)n(-c2ccc(OCc3ccccc3)nc2OCc2ccccc2)c2ccc(Br)cc21 ; CC(C)(C)OC(=O)Nc1ccc(N)cc1 ; Cn1c(=O)n(-c2ccc(OCc3ccccc3)nc2OCc2ccccc2)c2ccc(Nc3ccc(NC(=O)OC(C)(C)C)cc3)cc21 ; Cn1c(=O)n(C2CCC(=O)NC2=O)c2ccc(Nc3ccc(NC(=O)OC(C)(C)C)cc3)cc21 ; Cn1c(=O)n(C2CCC(=O)NC2=O)c2ccc(Nc3ccc(N)cc3)cc21]

### Step 1: tert-butyl N-[[4-[[1-(2,6-dibenzyloxy-3-pyridyl)-3-methyl-2-oxo-benzimidazol-5-yl]amino]phenyl]carbamate (2)

Into a 50 mL pressure tube containing a well-stirred solution of 5-bromo-1-(2,6-dibenzyloxy-3-pyridyl)-3-methyl-benzimidazol-2-one (1, 800 mg, 1.55 mmol) and tert-butyl N-(4-aminophenyl)carbamate (1a, 419.43 mg, 2.01 mmol, 7.24 μL) in 1,4-dioxane (10 mL) was added cesium carbonate (1.51 g, 4.65 mmol). The reaction mixture was degassed with nitrogen for 5 min. XPhos (73.85 mg, 154.92 μmol) and tris(dibenzylideneacetone)dipalladium(0) (283.73 mg, 309.85 μmol) were added. After 4 h at 90° C., the reaction mixture was filtered through Celite, washing with ethyl acetate. The filtrate was evaporated under reduced pressure, and the residue was purified by flash silica gel column chromatography (40% ethylacetate in pet-ether) to afford tert-butyl N-[[4-[[1-(2,6-dibenzyloxy-3-pyridyl)-3-methyl-2-oxo-benzimidazol-5-yl]amino]phenyl]carbamate (2, 870 mg, 1.34 mmol, 86% yield) as a color solid. LCMS (ES+): m/z 644.2 [M+H]⁺

### Step 2: tert-butyl N-[[4-[[1-(2,6-dioxo-3-piperidyl)-3-methyl-2-oxo-benzimidazol-5-yl]amino]phenyl]carbamate (3)

Into a 50 mL round bottom flask containing a well-stirred solution of tert-butyl N-[[4-[[1-(2,6-dibenzyloxy-3-pyridyl)-3-methyl-2-oxo-benzimidazol-5-yl]amino]phenyl]carbamate (2, 700 mg, 1.08 mmol) in 1,4-dioxane (7 mL) was added palladium hydroxide (20 wt. % on carbon) (758.95 mg, 5.40 mmol). The resulting suspension was stirred under hydrogen atmosphere at RT. After 20 h, the reaction mixture was filtered through Celite and washed with 1,4-dioxane (300 mL). The filtrate was concentrated under reduced pressure and the residue triturated with diethyl ether (20 mL), filtered and dried to afford tert-butyl N-[[4-[[1-(2,6-dioxo-3-piperidyl)-3-methyl-2-oxo-benzimidazol-5-yl]amino]phenyl]carbamate (3, 500 mg, 994.95 μmol, 92% yield) as an off white solid. LCMS (ES+): m/z 466.0 [M+H]⁺

### Step 3: 3-[5-(4-aminoanilino)-3-methyl-2-oxo-benzimidazol-1-yl]piperidine-2,6-dione (4)

Into a 25 mL single-neck round bottom flask containing a well-stirred solution of tert-butyl N-[4-[[1-(2,6-dioxo-3-piperidyl)-3-methyl-2-oxo-benzimidazol-5-yl]amino]phenyl]carbamate (3, 250 mg, 494.09 μmol) in DCM (2 mL) was added trifluoroacetic acid (1.69 g, 14.82 mmol, 1.14 mL) at 0° C. The reaction mixture was stirred at RT for 2 h. The volatiles were removed under reduced pressure and co-evaporated with DCM and toluene. The residue was washed with diethyl ether to afford 3-[5-(4-aminoanilino)-3-methyl-2-oxo-benzimidazol-1-yl]piperidine-2,6-dione (4, 250 mg, 452.17 μmol, 92% yield, TFA Salt) as a light brown solid. LCMS (ES+): m/z 366.1 [M+H]⁺

### 2-[4-[1-(2,6-dioxo-3-piperidyl)-3-methyl-2-oxo-benzimidazol-5-yl]-1-piperidyl]acetaldehyde (4)

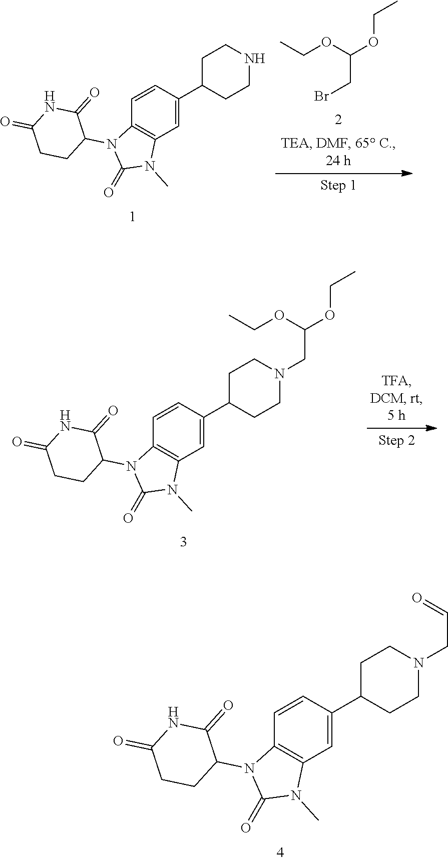

### Step 1: 3-[5-[1-(2,2-diethoxyethyl)-4-piperidyl]-3-methyl-2-oxo-benzimidazol-1-yl]piperidine-2,6-dione (3)

Into a 50 mL vial containing a well-stirred solution of 3-(3-methyl-2-oxo-5-(piperidin-4-yl)-2,3-dihydro-1H-benzo[d]imidazol-1-yl)piperidine-2,6-dione (1, 1 g, 2.64 mmol, HCl salt) in anhydrous DMF (10 mL) were added TEA (801.29 mg, 7.92 mmol, 1.10 mL) and 2-bromo-1,1-diethoxy-ethane (2, 1.04 g, 5.28 mmol). After 16 h at 65° C. the solvent was removed under reduced pressure and the residue purified by reverse phase column chromatography [Purification method: Silicycle C18 column; Mobile phase: 0.1% TFA in water; Mobile phase B: MeCN) to obtain 3-[5-[1-(2,2-diethoxyethyl)-4-piperidyl]-3-methyl-2-oxo-benzimidazol-1-yl]piperidine-2,6-dione (3, 1.3 g, 1.92 mmol, 72% yield, TFA salt) as an off-white solid. LCMS (ES+): m/z 459.1 [M+H]⁺

### Step 2: 2-[4-[1-(2,6-dioxo-3-piperidyl)-3-methyl-2-oxo-benzimidazol-5-yl]-1-piperidyl]acetaldehyde (4)

Into a 50 mL single neck round bottom flask containing a well-stirred solution of 3-[5-[1-(2,2-diethoxyethyl)-4-piperidyl]-3-methyl-2-oxo-benzimidazol-1-yl]piperidine-2,6-dione (3, 150 mg, 222.68 μmol, TFA salt) in anhydrous DCM (5 mL) was added TFA (2.52 g, 22.07 mmol, 1.70 mL). After 10 h, the solvent was removed under reduced pressure and the residue triturated with diethyl ether (50 mL), filtered and dried to afford 2-[4-[1-(2,6-dioxo-3-piperidyl)-3-methyl-2-oxo-benzimidazol-5-yl]-1-piperidyl]acetaldehyde (4, 100 mg, 150.47 μmol, 68% yield, TFA salt) as off white solid. LCMS (ES+): m/z 385.0 [M+H]⁺

### 3-[5-(4-aminophenyl)-3-methyl-2-oxo-benzimidazol-1-yl]piperidine-2,6-dione (4)

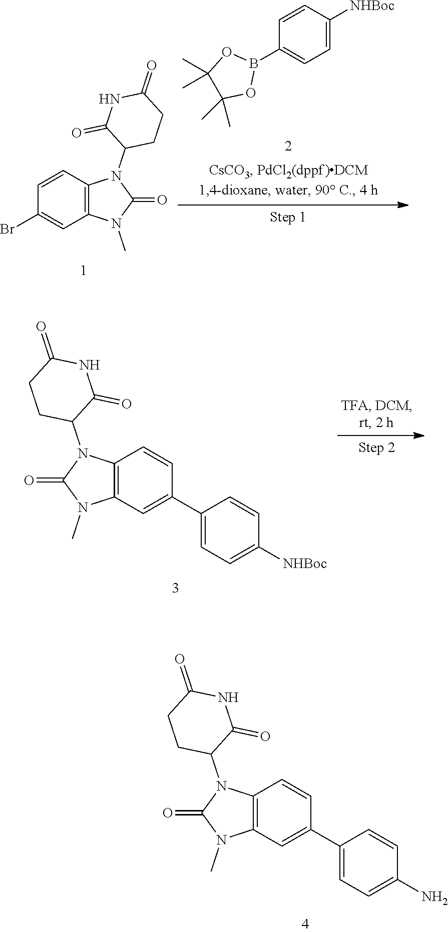

### Step 1: tert-butyl N-[[4-[[1-(2,6-dioxo-3-piperidyl)-3-methyl-2-oxo-benzimidazol-5-yl]phenyl]carbamate (3)

Into a 25 mL sealed tube containing a well-stirred solution of 3-(5-bromo-3-methyl-2-oxo-benzimidazol-1-yl)piperidine-2,6-dione (1, 800 mg, 2.37 mmol) and tert-butyl N-[4-(4,4,5,5-tetramethyl-1,3,2-dioxaborolan-2-yl)phenyl]carbamate (2, 922.97 mg, 2.60 mmol) in 1,4-dioxane (10 mL) and water (0.4 mL) was added cesium carbonate (924.98 mg, 2.84 mmol). The solution was degassed with nitrogen for 5 min. Then, Pd(dppf)Cl₂·DCM (289.64 mg, 354.87 μmol) was added. The reaction mixture was degassed for another 5 min. The tube was sealed and the reaction mixture was heated to 90° C. After 4 h, the reaction mixture was filtered through Celite, washed with ethyl acetate (100 mL) and the filtrate concentrated under reduced pressure. The material was purified by reversed phase column chromatography [Purification method: Siliasep premium C18, 120 g column; Mobile phase A: 0.1% TFA in water; Mobile phase B: Acetonitrile) to afford tert-butyl N-[[4-[[1-(2,6-dioxo-3-piperidyl)-3-methyl-2-oxo-benzimidazol-5-yl]phenyl]carbamate (3, 500 mg, 1.10 mmol, 46% yield) as an off white solid. LCMS (ES+): m/z 451.0 [M+H]⁺

### Step 2: 3-[5-(4-aminophenyl)-3-methyl-2-oxo-benzimidazol-1-yl]piperidine-2,6-dione (4)

Into a 50 mL single neck round bottom flask containing a well-stirred solution of tert-butyl N-[4-[1-(2,6-dioxo-3-piperidyl)-3-methyl-2-oxo-benzimidazol-5-yl]phenyl]carbamate (3, 500 mg, 1.09 mmol) in DCM (3 mL) was added TFA (1.86 g, 16.32 mmol, 1.26 mL) at 0° C. The reaction mixture was stirred at RT. After 2 h, the volatiles were removed under reduced pressure, the residue was triturated with diethyl ether (14 mL), filtered and dried to afford 3-[5-(4-aminophenyl)-3-methyl-2-oxo-benzimidazol-1-yl]piperidine-2,6-dione TFA salt (4, 350 mg, 717.49 μmol, 66% yield) as a pale yellow solid. LCMS (ES+): m/z 351.0 [M+H]⁺

### 2-[4-[1-(2,6-dioxo-3-piperidyl)-3-methyl-2-oxo-benzimidazol-5-yl]-3,3-difluoro-1-piperidyl]acetic acid (3)

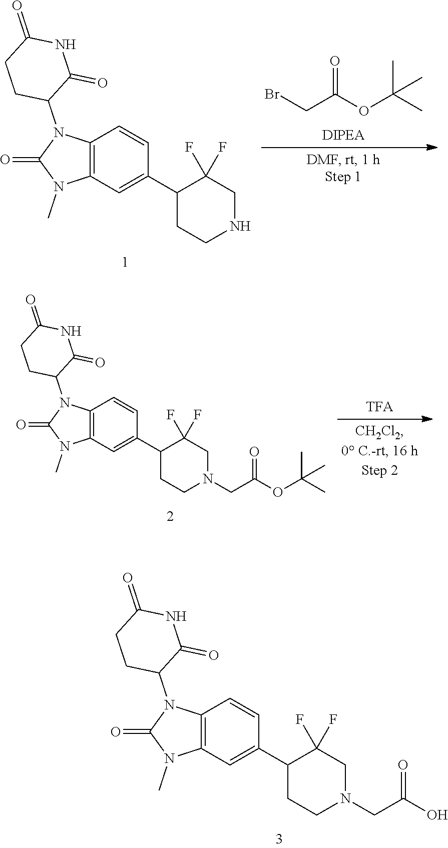

### Step 1: tert-butyl 2-[4-[1-(2,6-dioxo-3-piperidyl)-3-methyl-2-oxo-benzimidazol-5-yl]-3,3-difluoro-1-piperidyl]acetate (2)

In to a 50 mL single neck, round bottom flask containing a well-stirred solution of 3-[5-(3,3-difluoro-4-piperidyl)-3-methyl-2-oxo-benzimidazol-1-yl]piperidine-2,6-dione (1, 1.18 g, 2.18 mmol, TFA salt) in anhydrous DMF (10 mL) was added DIPEA (845.53 mg, 6.54 mmol, 1.14 mL) followed by tert-butyl 2-bromoacetate (425.37 mg, 2.18 mmol, 319.83 μL) at 0° C. After 1 h at RT, the reaction mixture was diluted with ice cold water (50 mL) to afford a solid which was dissolved in DCM (25 mL). The organic layer was separated, dried over Na₂SO₄, concentrated and triturated with Et₂O to afford tert-butyl 2-[4-[1-(2,6-dioxo-3-piperidyl)-3-methyl-2-oxo-benzimidazol-5-yl]-3,3-difluoro-1-piperidyl]acetate (2, 870 mg, 1.76 mmol, 79% yield) as a white solid. LCMS (ES+): m/z 493.0 [M+H]⁺

### Step 2: 2-[4-[1-(2,6-dioxo-3-piperidyl)-3-methyl-2-oxo-benzimidazol-5-yl]-3,3-difluoro-1-piperidyl]acetic acid (3)

Into a 25 mL single neck, round bottom flask containing a well-stirred solution of tert-butyl 2-[4-[1-(2,6-dioxo-3-piperidyl)-3-methyl-2-oxo-benzimidazol-5-yl]-3,3-difluoro-1-piperidyl]acetate (2, 300 mg, 584.75 μmol) in anhydrous DCM (2.5 mL) was added dropwise TFA (3.70 g, 32.45 mmol, 2.5 mL) at 0° C. After 16 h at RT, the solvent was removed and the material was triturated with Et₂O (10 mL) to afford 2-[4-[1-(2,6-dioxo-3-piperidyl)-3-methyl-2-oxo-benzimidazol-5-yl]-3,3-difluoro-1-piperidyl]acetic acid (3, 310 mg, 554.66 μmol, 95% yield, TFA salt) as an off-white solid. LCMS (ES+): m/z 437.2 [M+H]⁺

### 5-[4-(aminomethyl)anilino]-1-(6-benzyloxy-2-hydroxy-3-pyridyl)-3-methyl-benzimidazol-2-one (4)

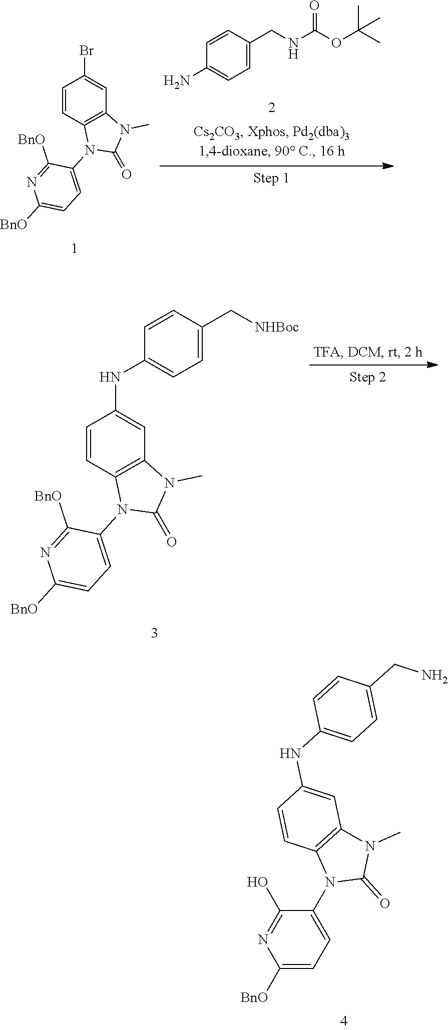 [SMILES: Cn1c(=O)n(-c2ccc(OCc3ccccc3)nc2OCc2ccccc2)c2ccc(Br)cc21 ; CC(C)(C)OC(=O)NCc1ccc(N)cc1 ; Cn1c(=O)n(-c2ccc(OCc3ccccc3)nc2OCc2ccccc2)c2ccc(Nc3ccc(CNC(=O)OC(C)(C)C)cc3)cc21 ; Cn1c(=O)n(-c2ccc(OCc3ccccc3)nc2O)c2ccc(Nc3ccc(CN)cc3)cc21]

### Step 1: tert-butyl N-[[4-[[1-(2,6-dibenzyloxy-3-pyridyl)-3-methyl-2-oxo-benzimidazol-5-yl]amino]phenyl]methyl]carbamate (3)

Into a 100 mL pressure tube containing a well-stirred solution of 5-bromo-1-(2,6-dibenzyloxy-3-pyridyl)-3-methyl-benzimidazol-2-one (1, 800 mg, 1.49 mmol) and tert-butyl N-[(4-aminophenyl)methyl]carbamate (2, 397.00 mg, 1.79 mmol) in 1,4-dioxane (16 mL) was added cesium carbonate (1.45 g, 4.47 mmol). The reaction mixture was degassed with nitrogen gas for 5 min. XPhos (70.95 mg, 148.83 μmol) and tris(dibenzylideneacetone)dipalladium(0) (68.15 mg, 74.42 μmol) were added and the reaction mixture was heated at 90° C. After 16 h, the reaction mixture was filtered through Celite, washing with EtOAc (50 mL). The filtrate was concentrated under reduced pressure and purified by flash silica gel column chromatography (35-40% EtOAc in pet ether) to afford tert-butyl N-[[4-[[1-(2,6-dibenzyloxy-3-pyridyl)-3-methyl-2-oxo-benzimidazol-5-yl]amino]phenyl]methyl]carbamate (3, 815 mg, 1.23 mmol, 82% yield) as a light brown solid. LCMS (ES+): m/z 658.0 [M+H]⁺

### Step 2: 5-[4-(aminomethyl)anilino]-1-(6-benzyloxy-2-hydroxy-3-pyridyl)-3-methyl-benzimidazol-2-one (4)

Into a 25 mL single neck round bottom flask containing a well-stirred solution of tert-butyl N-[[4-[[1-(2,6-dibenzyloxy-3-pyridyl)-3-methyl-2-oxo-benzimidazol-5-yl]amino]phenyl]methyl]carbamate (3, 800 mg, 1.00 mmol) in DCM (4 mL) was added TFA (2.96 g, 25.96 mmol, 2 mL) at 0° C. After 2 h the volatiles were removed under reduced pressure to afford a residue which was triturated with Et₂O (40 mL), filtered and dried to afford 5-[4-(aminomethyl)anilino]-1-(6-benzyloxy-2-hydroxy-3-pyridyl)-3-methyl-benzimidazol-2-one (4, 575 mg, 940.20 μmol, 94% yield, TFA salt) as an off-white solid. LCMS (ES+): m/z 468.0 [M+H]⁺

### 2-[4-[1-(2,6-dioxo-3-piperidyl)-3-methyl-2-oxo-benzimidazol-5-yl]piperazin-1-yl]acetic acid (7)

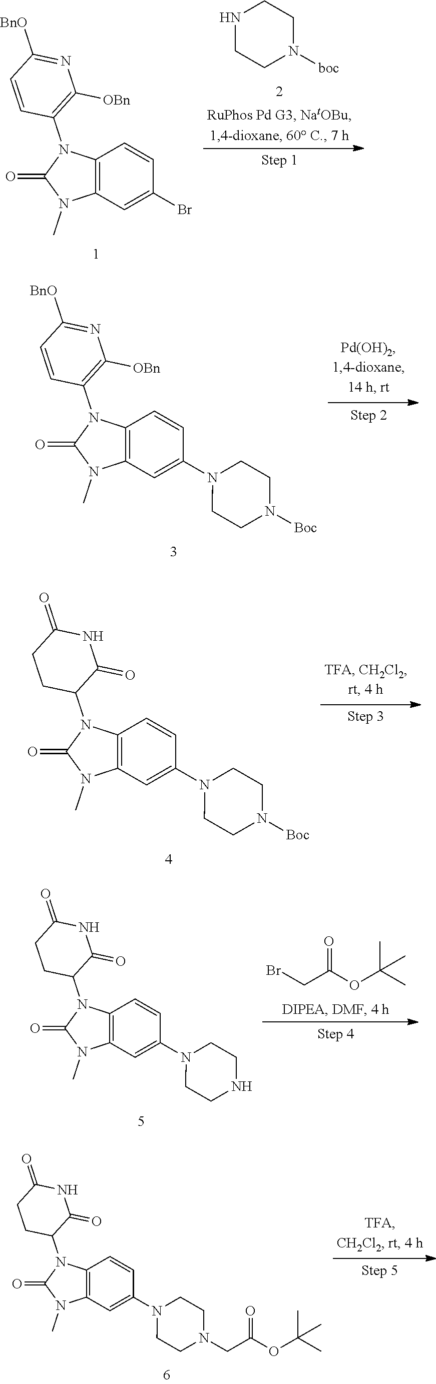

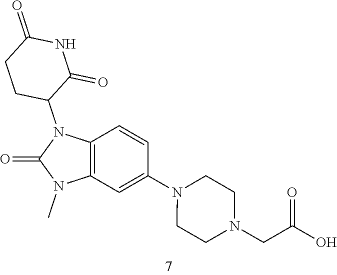 [SMILES: Cn1c(=O)n(C2CCC(=O)NC2=O)c2ccc(N3CCN(CC(=O)O)CC3)cc21]

### Step 1: tert-butyl 4-[1-(2,6-dibenzyloxy-3-pyridyl)-3-methyl-2-oxo-benzimidazol-5-yl]piperazine-1-carboxylate (3)

To a 50 mL pressure tube containing a solution of 5-bromo-1-(2,6-dibenzyloxy-3-pyridyl)-3-methyl-benzimidazol-2-one (1, 1.2 g, 2.02 mmol) in 1,4-dioxane (15 mL) was added tert-butyl piperazine-1-carboxylate (2, 376.55 mg, 2.02 mmol), sodium tert-butoxide (582.87 mg, 6.07 mmol). The mixture was degassed with nitrogen for 10 min. RuPhos Pd G3 (169.09 mg, 202.17 μmol) was added and degassed for another 5 min. The pressure tube was sealed and stirred at 90° C. After 7 h, the reaction mixture was filtered through Celite and washed with 1,4-dioxane (40 mL) and EtOAc (100 mL). The filtrate was concentrated under vacuum and the residue was purified by flash silica gel column chromatography (70% EtOAc in petroleum ether) to obtain tert-butyl 4-[1-(2,6-dibenzyloxy-3-pyridyl)-3-methyl-2-oxo-benzimidazol-5-yl]piperazine-1-carboxylate (3, 1.05 g, 1.57 mmol, 78% yield) as a yellow solid. LCMS (ES+): m/z 622.4 [M+H]⁺

### Step 2: tert-butyl 4-[1-(2,6-dioxo-3-piperidyl)-3-methyl-2-oxo-benzimidazol-5-yl]piperazine-1-carboxylate (4)

To a 100 mL single neck round bottom flask containing a well-stirred solution of tert-butyl 4-[1-(2,6-dibenzyloxy-3-pyridyl)-3-methyl-2-oxo-benzimidazol-5-yl]piperazine-1-carboxylate (3, 1.05 g, 1.57 mmol) in 1,4-dioxane (20 mL) was added palladium hydroxide (20% on carbon) (1.05 g, 1.49 mmol, 20% purity). The suspension was stirred under hydrogen atmosphere at RT. After 14 h the reaction mixture was filtered through Celite and washed with 1,4-dioxane (400 mL) and DCM (150 mL). The filtrate was concentrated under reduced pressure to afford tert-butyl 4-[1-(2,6-dioxo-3-piperidyl)-3-methyl-2-oxo-benzimidazol-5-yl]piperazine-1-carboxylate (4, 695 mg, 1.52 mmol, 97% yield) as a brown solid. The material was used in the next step without purification. LCMS (ES+): m/z 444.3 [M+H]⁺

### Step 3: 3-(3-methyl-2-oxo-5-piperazin-1-yl-benzimidazol-1-yl)piperidine-2,6-dione (5)

To a 50 mL single neck round bottom flask containing a solution of tert-butyl 4-[1-(2,6-dioxo-3-piperidyl)-3-methyl-2-oxo-benzimidazol-5-yl]piperazine-1-carboxylate (4, 695 mg, 1.52 mmol) in DCM (5 mL) was added trifluoroacetic acid (2.96 g, 25.96 mmol, 2 mL) at 0° C. and the reaction mixture was stirred at ambient temperature. After 4 h. the solvent was removed under reduced pressure, co-distilled with DCM (5 mL) and toluene (2×10 mL), dried under vacuum to afford 3-(3-methyl-2-oxo-5-piperazin-1-yl-benzimidazol-1-yl)piperidine-2,6-dione (5, 605 mg, 1.24 mmol, 82% yield, TFA salt) as a dark brown sticky solid. LCMS (ES+): m/z 344.2 [M+H]⁺

### Step 4: tert-butyl 2-[4-[1-(2,6-dioxo-3-piperidyl)-3-methyl-2-oxo-benzimidazol-5-yl]piperazin-1-yl]acetate (6)

To a 50 mL single neck round bottom flask containing a solution of 3-(3-methyl-2-oxo-5-piperazin-1-yl-benzimidazol-1-yl)piperidine-2,6-dione (5, 600 mg, 1.23 mmol. TFA salt) in anhydrous DMF (5 mL) was added N,N-diisopropylethylamine (318.73 mg, 2.47 mmol, 429.55 μL) under nitrogen atmosphere. The resulting suspension was cooled to 0° C. and tert-butyl 2-bromoacetate (264.56 mg, 1.36 mmol, 198.92 μL) was added dropwise. The reaction mixture was stirred at ambient temperature for 4 h. The reaction mixture was diluted with DCM (50 mL), washed with water (2×25 mL) and brine solution (25 mL), dried over sodium sulfate, filtered and solvent to obtain removed tert-butyl 2-[4-[1-(2,6-dioxo-3-piperidyl)-3-methyl-2-oxo-benzimidazol-5-yl]piperazin-1-yl]acetate (6, 600 mg, 826.19 μmol, 67% yield) as a brown gummy solid. The material was used in the next step without purification. LCMS (ES+): m/z 458.1 [M+H]⁺

### Step 5: 2-[4-[1-(2,6-dioxo-3-piperidyl)-3-methyl-2-oxo-benzimidazol-5-yl]piperazin-1-yl]acetic acid (7)

To a 50 mL single-neck round bottom flask containing a solution of tert-butyl 2-[4-[1-(2,6-dioxo-3-piperidyl)-3-methyl-2-oxo-benzimidazol-5-yl]piperazin-1-yl]acetate (6, 600 mg, 826.19 μmol) in DCM (3 mL) was added trifluoroacetic acid (2.96 g, 25.96 mmol, 2 mL) at 0)° C. The reaction mixture was stirred at ambient temperature for 3 h. The solvent was evaporated to dryness, co-distilled with toluene (2×10 mL), triturated with diethyl ether (20 mL), filtered and dried under vacuum to afford 2-[4-[1-(2,6-dioxo-3-piperidyl)-3-methyl-2-oxo-benzimidazol-5-yl]piperazin-1-yl]acetic acid (7, 570 mg, 774.10 μmol, 94% yield, TFA salt) as a black sticky solid. LCMS (ES+): m/z 401.9 [M+H]⁺

### 2-[4-[1-(2,6-dioxo-3-piperidyl)-2-oxo-benzo[cd]indol-6-yl]-3,3-difluoro-1-piperidyl]acetic acid (4)

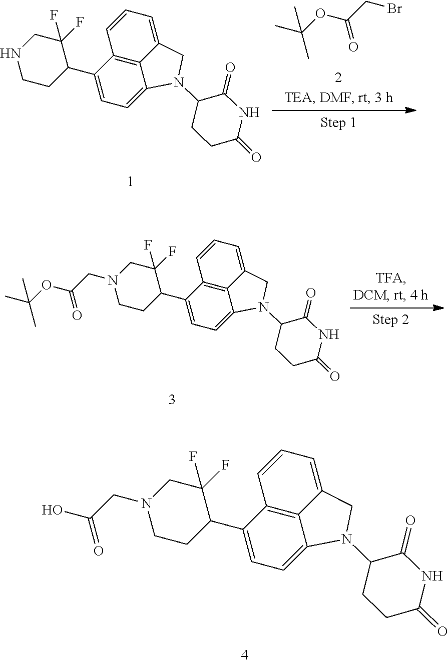

### Step 1: tert-butyl 2-[4-[1-(2,6-dioxo-3-piperidyl)-2-oxo-benzo[cd]indol-6-yl]-3,3-difluoro-1-piperidyl]acetate (3)

Into a 50 mL single neck round bottom flask containing a well-stirred solution of 3-[6-(3,3-difluoro-4-piperidyl)-2-oxo-benzo[cd]indol-1-yl]piperidine-2,6-dione (1, 300 mg, 531.74 μmol) in DMF (4 mL) were added triethylamine (269.03 mg, 2.66 mmol, 370.57 μL) and tert-butyl 2-bromoacetate (2, 124.46 mg, 638.08 μmol, 93.58 μL) at 0° C. The reaction mixture was stirred at RT. After 3 h, cold water (3 mL) was added and the solid was filtered and washed with cold water (3 mL) and diethyl ether (2 mL) to afford tert-butyl 2-[4-[1-(2,6-dioxo-3-piperidyl)-2-oxo-benzo[cd]indol-6-yl]-3,3-difluoro-1-piperidyl]acetate (3, 200 mg, 374.19 μmol, 70% yield) as a pale yellow solid. LCMS (ES+): m/z 514.2 [M+H]⁺

### Step 2: 2-[4-[1-(2,6-dioxo-3-piperidyl)-2-oxo-benzo[cd]indol-6-yl]-3,3-difluoro-1-piperidyl]acetic acid (4)

Into a 50 mL single neck round bottom flask containing a well-stirred solution of tert-butyl 2-[4-[1-(2,6-dioxo-3-piperidyl)-2-oxo-benzo[cd]indol-6-yl]-3,3-difluoro-1-piperidyl]acetate (50 mg, 36.03 μmol) in DCM (2 mL) was added trifluoroacetic acid (63.65 mg, 558.27 μmol, 43.01 μL) at 0° C. The reaction mixture was stirred at RT for 4 h. The volatiles were removed under reduced pressure and the residue purified by reverse phase prep HPLC [Purification method: Column: Xbridge-C-18 (19×150 mm), 5 micron; Mobile phase A: 0.1% TFA in water; Mobile phase B: MeCN] to afford 2-[4-[1-(2,6-dioxo-3-piperidyl)-2-oxo-benzo[cd]indol-6-yl]-3,3-difluoro-1-piperidyl]acetic acid (4, 15 mg, 25.96 μmol, 72% yield, TFA salt) as an off white solid. LCMS (ES+): m/z 458.0 [M+H]⁺

### (3-(1-(2,6-dioxopiperidin-3-yl)-2-oxo-1,2-dihydrobenzo[cd]indol-6-yl)propyl)glycine (5)

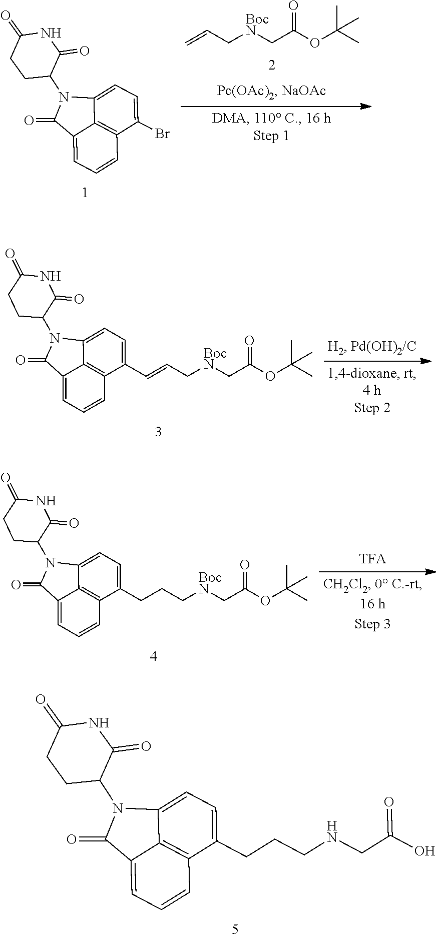 [SMILES: O=C1CCC(N2C(=O)c3cccc4c(Br)ccc2c34)C(=O)N1 ; C=CCN(CC(=O)OC(C)(C)C)C(=O)OC(C)(C)C ; CC(C)(C)OC(=O)CN(C/C=C/c1ccc2c3c(cccc13)C(=O)N2C1CCC(=O)NC1=O)C(=O)OC(C)(C)C ; CC(C)(C)OC(=O)CN(CCCc1ccc2c3c(cccc13)C(=O)N2C1CCC(=O)NC1=O)C(=O)OC(C)(C)C ; O=C(O)CNCCCc1ccc2c3c(cccc13)C(=O)N2C1CCC(=O)NC1=O]

### Step 1: tert-butyl 2-[tert-butoxycarbonyl-[(E)-3-[1-(2,6-dioxo-3-piperidyl)-2-oxo-benzo[cd]indol-6-yl]allyl]amino]acetate (3)

A 20 mL sealed tube containing a well-stirred solution of 3-(6-bromo-2-oxo-benzo[cd]indol-1-yl)piperidine-2,6-dione (1, 1 g, 2.58 mmol) and tert-butyl 2-[allyl(tert-butoxycarbonyl)amino]acetate (2, 2.01 g, 6.44 mmol) in DMA (6 mL) was degassed with nitrogen for 5 min. Then Pd(OAc)₂ (173.46 mg, 772.61 μmol) and NaOAc (422.51 mg, 5.15 mmol) were added and the mixture was heated at 110° C. for 16 h. The reaction mixture was concentrated under reduced pressure and purified by reverse phase column chromatography [Column: Redisep-C18-120 g; Mobile Phase A: 0.1% NH₄OAc in water and Mobile Phase B: CH₃CN] to afford tert-butyl 2-[tert-butoxycarbonyl-[(E)-3-[1-(2,6-dioxo-3-piperidyl)-2-oxo-benzo[cd]indol-6-yl]allyl]amino]acetate (3, 800 mg, 1.25 mmol, 49% yield) as pale yellow solid. LCMS (ES−): m/z 548.3 [M−H]⁻

### Step 2: tert-butyl 2-[tert-butoxycarbonyl-[3-[1-(2,6-dioxo-3-piperidyl)-2-oxo-benzo[cd]indol-6-yl]propyl]amino]acetate (4)

Into a 100 mL single neck round bottom flask containing a well-stirred solution of tert-butyl 2-[tert-butoxycarbonyl-[(E)-3-[1-(2,6-dioxo-3-piperidyl)-2-oxo-benzo[cd]indol-6-yl]allyl]amino]acetate (3, 800 mg, 1.25 mmol) in anhydrous 1,4-dioxane (30 mL) was added Pd(OH)₂ (20 wt. %, 50% water, on carbon) (800 mg, 5.70 mmol). The reaction mixture was stirred for 4 h under hydrogen gas bladder pressure. The reaction mixture filtered through Celite, washed with 1,4-dioxane (40 mL), and concentrated under reduced pressure. Purification by reverse-phase column chromatography [Column: Redisep-C18-120 g; Mobile Phase A: 0.1% NH₄OAc in water and Mobile Phase B: CH₃CN] afforded tert-butyl 2-[tert-butoxycarbonyl-[3-[1-(2,6-dioxo-3-piperidyl)-2-oxo-benzo[cd]indol-6-yl]propyl]amino]acetate (4, 600 mg, 1.08 mmol, 87% yield) as yellow solid. LCMS (ES−): m/z 550.3 [M−H]⁻

### Step 3: (3-(1-(2,6-dioxopiperidin-3-yl)-2-oxo-1,2-dihydrobenzo[cd]indol-6-yl)propyl)glycine (5)

Into a 50 mL single neck, round bottom flask containing a well-stirred solution of tert-butyl 2-[tert-butoxycarbonyl-[3-[1-(2,6-dioxo-3-piperidyl)-2-oxo-benzo[cd]indol-6-yl]propyl]amino]acetate (4, 200 mg, 358.94 μmol) in dry DCM (10 mL) was added TFA (818.52 mg, 7.18 mmol, 553.05 μL) at 0° C. The reaction mixture was stirred at RT for 16 h. The reaction mixture was concentrated under reduced pressure and triturated with MTBE (10 mL) to afford (3-(1-(2,6-dioxopiperidin-3-yl)-2-oxo-1,2-dihydrobenzo[cd]indol-6-yl)propyl)glycine (5, 170 mg, 275.64 μmol, 77% yield, TFA salt) as yellow solid. LCMS (ES+): m/z 396.2 [M+H]⁺

### (R)-2-(1-(3-(2,6-dioxopiperidin-3-yl)-1-methyl-1H-indazol-6-yl)piperidin-4-yl)acetic acid (5a, ambiguously assigned) and (S)-2-(1-(3-(2,6-dioxopiperidin-3-yl)-1-methyl-1H-indazol-6-yl)piperidin-4-yl)acetic acid (5b, Ambiguously Assigned)

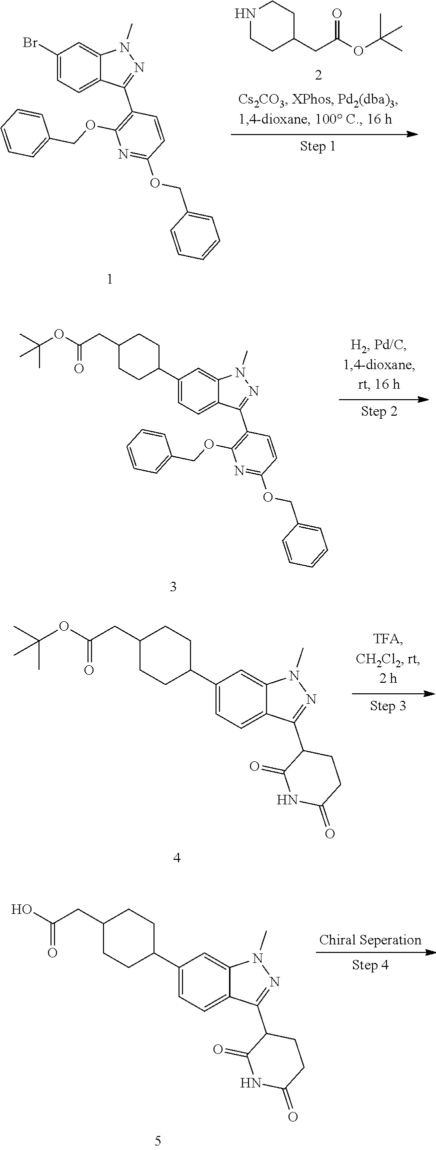 [SMILES: Cn1nc(-c2ccc(OCc3ccccc3)nc2OCc2ccccc2)c2ccc(Br)cc21 ; CC(C)(C)OC(=O)CC1CCNCC1 ; Cn1nc(-c2ccc(OCc3ccccc3)nc2OCc2ccccc2)c2ccc(C3CCC(CC(=O)OC(C)(C)C)CC3)cc21 ; Cn1nc(C2CCC(=O)NC2=O)c2ccc(C3CCC(CC(=O)OC(C)(C)C)CC3)cc21 ; Cn1nc(C2CCC(=O)NC2=O)c2ccc(C3CCC(CC(=O)O)CC3)cc21]

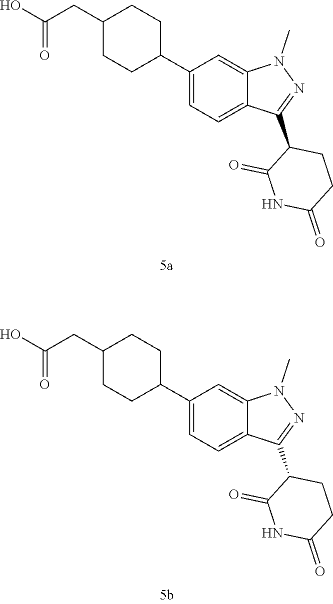 [SMILES: Cn1nc([C@H]2CCC(=O)NC2=O)c2ccc(C3CCC(CC(=O)O)CC3)cc21 ; Cn1nc([C@@H]2CCC(=O)NC2=O)c2ccc(C3CCC(CC(=O)O)CC3)cc21]

### Step 1: tert-butyl 2-[1-[3-(2,6-dibenzyloxy-3-pyridyl)-1-methyl-indazol-6-yl]-4-piperidyl]acetate (3)

Into a pressure tube containing a well-stirred solution of tert-butyl 2-(4-piperidyl)acetate (2, 449.30 mg, 2.25 mmol) in 1,4-dioxane (10 mL) was added 6-bromo-3-(2,6-dibenzyloxy-3-pyridyl)-1-methyl-indazole (1, 1.3 g, 2.25 mmol). The reaction mixture was degassed by bubbling nitrogen through for 5 min. Cesium carbonate (1.47 g, 4.51 mmol), XPhos (107.45 mg, 225.40 μmol) and Pd₂(dba)₃ (103.20 mg, 112.70 μmol) were added. The pressure tube was sealed and stirred at 100° C. After 16 h, the reaction mixture was filtered through Celite and washed with ethyl acetate (2×50 mL). The filtrate was concentrated under reduced pressure and purified by flash silica gel column chromatography (20% EtOAc in pet ether) to obtain tert-butyl 2-[1-[3-(2,6-dibenzyloxy-3-pyridyl)-1-methyl-indazol-6-yl]-4-piperidyl]acetate (3, 1.0 g, 1.43 mmol, 64% yield) as a colorless solid. LCMS (ES+): m/z 619.3 [M+H]⁺

### Step 2: tert-butyl 2-[1-[3-(2,6-dioxo-3-piperidyl)-1-methyl-indazol-6-yl]-4-piperidyl]acetate (4)

To a stirred solution of tert-butyl 2-[1-[3-(2,6-dibenzyloxy-3-pyridyl)-1-methyl-indazol-6-yl]-4-piperidyl]acetate (3, 1.1 g, 1.58 mmol) in 1,4-dioxane (15 mL) was added dihydroxypalladium (498.64 mg, 710.15 μmol, 20% purity) under nitrogen atmosphere. The suspension was stirred under hydrogen atmosphere with bladder pressure. After 16 h, the reaction mixture was filtered through Celite, washed with 50%1,4-dioxane in DCM (500 mL). The filtrate was concentrated under reduced pressure to afford tert-butyl 2-[1-[3-(2,6-dioxo-3-piperidyl)-1-methyl-indazol-6-yl]-4-piperidyl]acetate (4, 800 mg, 1.35 mmol, 86% yield). The material was used in the next step without purification. LCMS (ES+): m/z 441.3 [M+H]⁺

### Step 3: 2-[1-[3-(2,6-dioxo-3-piperidyl)-1-methyl-indazol-6-yl]-4-piperidyl]acetic acid (5)

To a stirred solution of tert-butyl 2-[1-[3-(2,6-dioxo-3-piperidyl)-1-methyl-indazol-6-yl]-4-piperidyl]acetate (4, 800 mg, 1.35 mmol) in CH₂Cl₂ (10 mL) was added trifluoroacetic acid (1.85 g, 16.19 mmol, 1.25 mL) at 0° C.) After 4 h at RT, the volatiles were evaporated and co-distilled with toluene to obtain 2-[1-[3-(2,6-dioxo-3-piperidyl)-1-methyl-indazol-6-yl]-4-piperidyl]acetic acid (5, 630 mg, 1.08 mmol, 80% yield, TFA salt) as a brown solid. LCMS (ES+): m/z 385.2 [M+H]⁺

### Step 4: (R)-2-(1-(3-(2,6-dioxopiperidin-3-yl)-1-methyl-1H-indazol-6-yl)piperidin-4-yl)acetic acid (5a, ambiguously assigned) and (S)-2-(1-(3-(2,6-dioxopiperidin-3-yl)-1-methyl-1H-indazol-6-yl)piperidin-4-yl)acetic acid (5b, Ambiguously Assigned)

2-[1-[3-(2,6-dioxo-3-piperidyl)-1-methyl-indazol-6-yl]-4-piperidyl]acetic acid (400 mg, 1.04 mmol) was resolved by normal phase chiral prep HPLC affording (R)-2-(1-(3-(2,6-dioxopiperidin-3-yl)-1-methyl-1H-indazol-6-yl)piperidin-4-yl)acetic acid (110 mg, 285.37 μmol, 27.43% yield, 99.73% purity) [as 1st eluent] and (S)-2-(1-(3-(2,6-dioxopiperidin-3-yl)-1-methyl-1H-indazol-6-yl)piperidin-4-yl)acetic acid 39.83 μmol, 23.05% yield, 97.05% purity) [as 2nd eluent] using a Chiralpak IC (250×21 mm) 5u column with DCM/IPA: 60/40 as the mobile phase, a flow rate of 18 ml/min and a run time of 18.0 minutes.

### 2-(6-((1-(2,6-dioxopiperidin-3-yl)-3-methyl-2-oxo-2,3-dihydro-1H-benzo[d]imidazol-5-yl)amino)pyridin-3-yl)acetic acid (7)

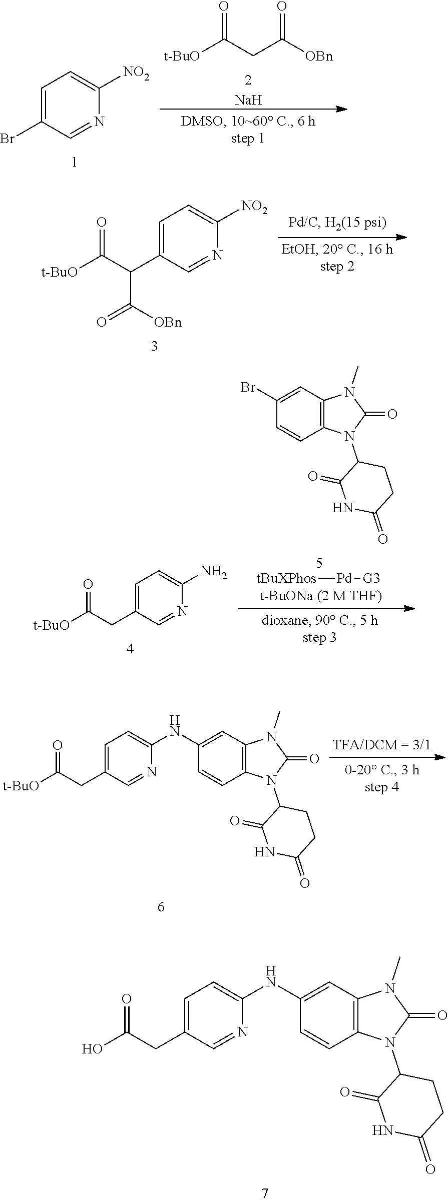

### Step 1: 1-benzyl 3-tert-butyl 2-(6-nitropyridin-3-yl)malonate (3)

A solution of benzyl tert-butyl malonate (2, 24.66 g, 98.53 mmol) in DMSO (200 mL) was treated with NaH (60 purity dispersion in mineral oil) (3.94 g, 10.28 mol) at 10° C. and stirred at 20° C. for 1 hour. 5-bromo-2-nitropyridine (1, 10 g, 49.26 mmol) was added to the mixture and stirred at 60° C. for 5 h. The reaction mixture was quenched with saturated NH₄Cl (600 mL), extracted with ethyl acetate (3×300 mL). The organic phases were combined and washed with brine (300 mL), dried over anhydrous Na₂SO₄, filtered and concentrated under reduced pressure. The residue was purified by reversed phase flash chromatography (flow: 200 mL/min; gradient: from 30%-60% MeCN-Water (0.1% TFA) over 40 min; column: I.D. 95 mm×H365 mm, Welch Ultimate XB_C18 20-40 μm: 120 Å) to afford 1-benzyl 3-tert-butyl 2-(6-nitropyridin-3-yl)malonate (3, 8 g, 21.27 mmol, 43% yield) as a yellow oil. LCMS (ESI): m/z 373.2 [M+H]⁺

### Step 2: tert-butyl 2-(6-aminopyridin-3-yl)acetate (4)

A solution of 1-benzyl 3-tert-butyl 2-(6-nitropyridin-3-yl)malonate (3, 8 g, 21.48 mmol) in Ethanol (100 mL) was added Pd/C (1.20 g) and stirred under H₂ atmosphere (15 Psi) for 16 h. The reaction mixture was filtered and concentrated under reduced pressure to afford tert-butyl 2-(6-aminopyridin-3-yl)acetate (4, 4.3 g, 20.03 mmol, 93% yield) as a yellow oil. The material was used in the next step without further purification. LCMS (ESI): m/z 209.2 [M+H]⁺

### Step 3: tert-butyl 2-(6-((1-(2,6-dioxopiperidin-3-yl)-3-methyl-2-oxo-2,3-dihydro-1H-benzo[d]imidazol-5-yl)amino)pyridin-3-yl)acetate (6)

A mixture of tert-butyl 2-(6-aminopyridin-3-yl)acetate (4, 1 g, 4.80 mmol) and 3-(5-bromo-3-methyl-2-oxo-2,3-dihydro-1H-benzo[d]imidazol-1-yl)piperidine-2,6-dione (5, 1.08 g, 3.20 mmol) in dioxane (15 mL) were added ^{t}BuXPhos-Pd-G3 (254.29 mg, 320.12 μmol) and t-BuONa (2 M in THF) (4.80 mL). The mixture was degassed and purged with N₂ 3 times, and then stirred at 90° C. for 5 h. The reaction mixture was cooled to RT and poured into saturated NH₄Cl (40 mL) and extracted with ethyl acetate (3×50 mL). The organic phases were combined and washed with brine (100 mL), dried over anhydrous Na₂SO₄, filtered and concentrated under reduced pressure. Half of the residue was purified by flash silica gel chromatography (Biotage®; 60 mL/min, Eluent of 0-70% ethyl acetate/petroleum ether gradient, Column: 4 g SepaFlash® Silica Flash) to give 60 mg crude product, which was purified by prep-HPLC (flow: 25 mL/min; gradient: from 14-44% MeCN-water (0.1% TFA) over 10 min; column: Phenomenex Synergi C18 150×25 mm×10 um) to afford tert-butyl 2-(6-((1-(2,6-dioxopiperidin-3-yl)-3-methyl-2-oxo-2,3-dihydro-1H-benzo[d]imidazol-5-yl)amino)pyridin-3-yl)acetate (6, 35 mg, 650.05 μmol, 4% yield, TFA salt) as a yellow solid. LCMS (ESI): m/z 466.1 [M+H]⁺

¹H NMR (400 MHZ, d₆-DMSO) δ 11.12 (s, 1H), 7.92-7.82 (m, 1H), 7.80-7.62 (m, 1H), 7.45 (s, 1H), 7.18-6.93 (m, 3H), 5.43-5.30 (m, 1H), 3.55 (d, J=4.0 Hz, 2H), 3.34 (s, 3H), 2.95-2.87 (m, 1H), 2.72-2.60 (m, 2H), 2.11-1.90 (m, 2H), 1.41 (s, 9H)

### Step 4: 2-(6-((1-(2,6-dioxopiperidin-3-yl)-3-methyl-2-oxo-2,3-dihydro-1H-benzo[d]imidazol-5-yl)amino)pyridin-3-yl)acetic acid (7)

To a solution of tert-butyl 2-(6-((1-(2,6-dioxopiperidin-3-yl)-3-methyl-2-oxo-2,3-dihydro-1H-benzo[d]imidazol-5-yl)amino)pyridin-3-yl)acetate (6, 220 mg, 42.96 μmol) in DCM (3 mL) was added TFA (1.63 g, 14.28 mmol, 1.10 mL) at 0° C. After 3 h at RT, the reaction mixture was filtered and concentrated under reduced pressure to afford 2-(6-((1-(2,6-dioxopiperidin-3-yl)-3-methyl-2-oxo-2,3-dihydro-1H-benzo[d]imidazol-5-yl)amino)pyridin-3-yl)acetic acid (7, 250 mg, 425.09 μmol, 90% yield, TFA salt) as a yellow solid. The material was used in the next step without further purification. LCMS (ESI): m/z 410.3 [M+H]⁺

### 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetic acid (6)

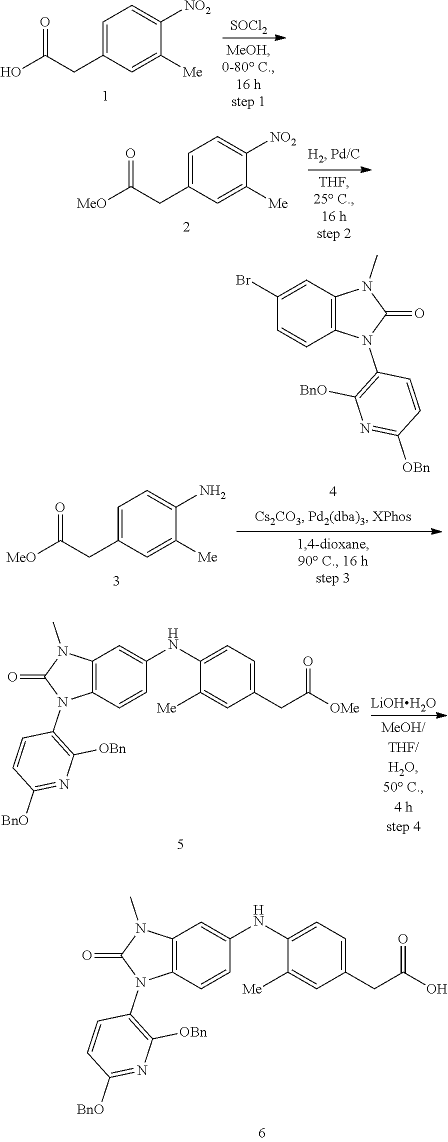

### Step 1: methyl 2-(3-methyl-4-nitrophenyl)acetate (2)

To a solution of 2-(3-methyl-4-nitrophenyl)acetic acid (1, 2 g, 10.25 mmol) in MeOH (40 mL) was added thionyl chloride (3.66 g, 30.74 mmol, 2.23 mL) dropwise at 0-10° C. After addition, the solution was stirred at 80° C. for 16 h. The reaction mixture was concentrated under reduced pressure and the residue was diluted with ethyl acetate (50 mL) and washed with sat. NaHCO₃ (50) mL×2) and brine (50 mL×2), dried over Na₂SO₄, filtered and concentrated under reduced pressure. The residue was purified by column chromatography (SiO₂, petroleum ether/ethyl acetate=50/1 to 20/1) to afford methyl 2-(3-methyl-4-nitrophenyl)acetate (2, 2 g, 9.56 mmol, 93% yield) as yellow oil.

¹H NMR (400 MHZ, CDCl₃) δ 7.96 (d, J=8.8 Hz, 1H), 7.26 (m, 2H), 3.72 (s, 3H), 3.67 (s, 2H), 2.60 (s, 3H).

### Step 2: methyl 2-(4-amino-3-methyl-phenyl)acetate (3)

To a solution of methyl 2-(3-methyl-4-nitrophenyl)acetate (2, 2 g, 9.56 mmol) in THF (30 mL) was added Pd/C (200 mg, 1.65 mmol, 10% purity). The mixture was stirred at 25° C. for 16 h under H₂ atmosphere (15 psi). The reaction mixture was filtered and washed with ethyl acetate (20 mL×3). The filtrate was concentrated under reduced pressure to afford methyl 2-(4-amino-3-methyl-phenyl)acetate (3, 1.5 g, 8.37 mmol, 88% yield) as yellow oil. The material was used in the next step without further purification. LCMS (ESI): m/z 180.1 [M+H]⁺

### Step 3: methyl 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetate (5)

To a mixture of methyl 2-(4-amino-3-methyl-phenyl)acetate (3, 160.00 mg, 892.78 μmol) and 1-(2,6-bis(benzyloxy)pyridin-3-yl)-5-bromo-3-methyl-1H-benzo[d]imidazol-2(3H)-one (4, 400 mg, 774.62 μmol) in dioxane (5 mL) were added Cs₂CO₃ (504.77 mg, 1.55 mmol). Xphos (36.93 mg, 77.46 μmol) and Pd₂(dba)₃ (70.93 mg, 77.46 μmol) under N₂. The mixture was stirred at 90° C. for 16 h. The reaction mixture was concentrated under reduced pressure and the residue was purified by column chromatography (SiO₂, petroleum ether/ethyl acetate=10/1 to 1/1) to afford methyl 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetate (5, 400 mg, 585.66 μmol, 76% yield) as a yellow solid. LCMS (ESI): m/z 615.2 [M+H]⁺

### Step 4: 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetic acid (6)

To a solution of methyl 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetate (5, 440 mg, 551.17 μmol) in H₂O (2 mL), THF (4 mL) and MeOH (4 mL) was added LiOH·H₂O (115.65 mg, 2.76 mmol). The mixture was stirred at 50° C. for 4 h. The reaction mixture was adjusted to pH 3 with IN HCl at 10-20° C.

The mixture was extracted with ethyl acetate (30 mL×2). The combined organic layers were washed with brine (30 mL×2), dried over Na₂SO₄, filtered and concentrated under reduced pressure. The residue was purified by prep-HPLC (flow: 60 mL/min; gradient: from 48-18% water (0.1% TFA) in MeCN over 10 min; column: Phenomenex luna C18 150×40 mm×15 μm) to afford 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetic acid (6, 300 mg, 494.45 μmol, 90% yield) as a yellow solid. LCMS (ESI): m/z 601.2 [M+H]⁺

### 2-[4-[1-(2,6-dioxo-3-piperidyl)-3-isopropyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 85)

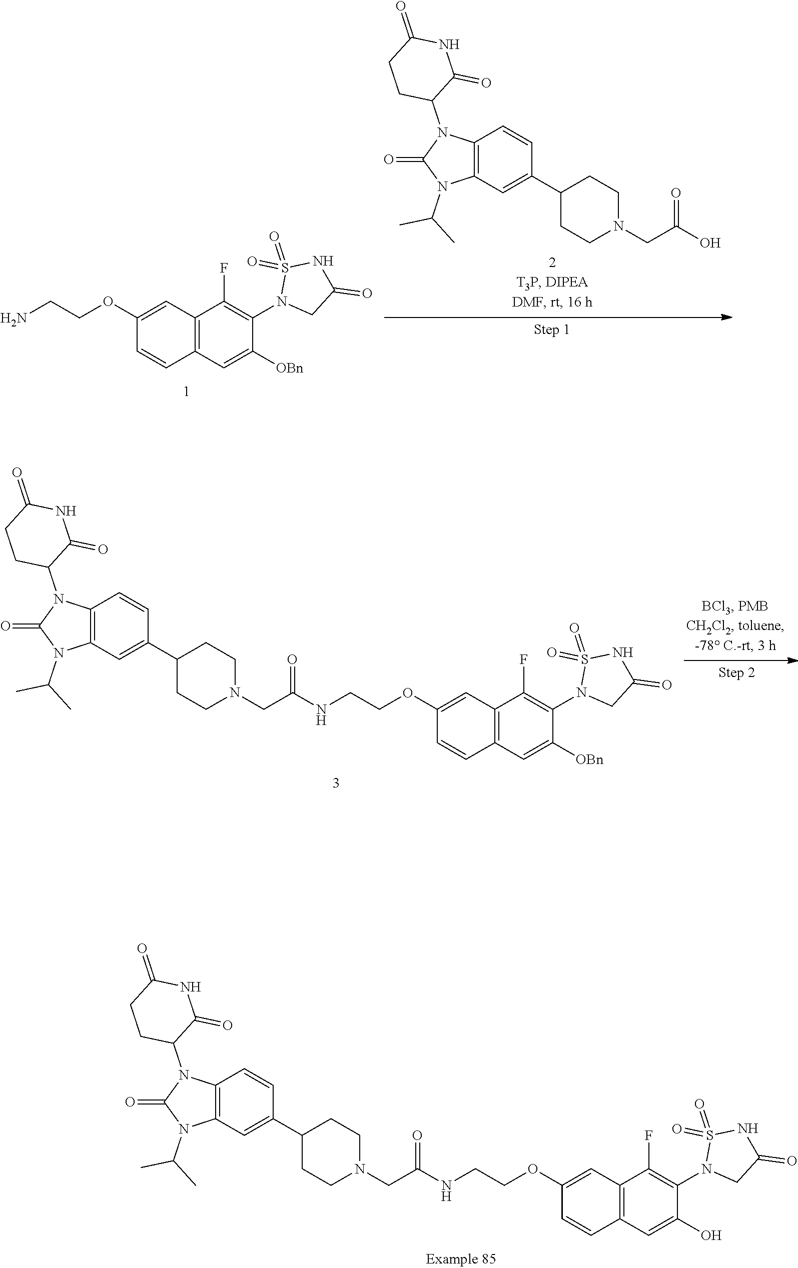 [SMILES: NCCOc1ccc2cc(OCc3ccccc3)c(N3CC(=O)NS3(=O)=O)c(F)c2c1 ; CC(C)n1c(=O)n(C2CCC(=O)NC2=O)c2ccc(C3CCN(CC(=O)O)CC3)cc21 ; CC(C)n1c(=O)n(C2CCC(=O)NC2=O)c2ccc(C3CCN(CC(=O)NCCOc4ccc5cc(OCc6ccccc6)c(N6CC(=O)NS6(=O)=O)c(F)c5c4)CC3)cc21 ; CC(C)n1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21]

### Step 1: N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-3-isopropyl-2-oxo-benzimidazol-5-yl]-1-piperidyl]acetamide (3)

Into a 10 mL single neck round bottom flask containing a well-stirred solution of 2-[4-[1-(2,6-dioxo-3-piperidyl)-3-isopropyl-2-oxo-benzimidazol-5-yl]-1-piperidyl]acetic acid (2, 180 mg, 331.80 μmol, TFA salt) in DMF (0.5 mL) were added DIPEA (214.41 mg, 1.66 mmol, 288.96 μL) and propylphosphonic anhydride solution (50 wt % in EtOAc) (422.28 mg, 663.59 μmol). After 1 h, 5-[7-(2-aminoethoxy)-3-benzyloxy-1-fluoro-2-naphthyl]-1,1-dioxo-1,2,5-thiadiazolidin-3-one (1, 162.56 mg, 364.97 μmol, TFA salt) was added and the reaction mixture was stirred for an additional 15 h. The reaction mixture was concentrated under reduced pressure and purified by reverse-phase column chromatography [C18 column-320 g); Mobile phase A: 0.1% Formic acid in water; Mobile phase B: MeCN] to obtain N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-3-isopropyl-2-oxo-benzimidazol-5-yl]-1-piperidyl]acetamide (3, 214 mg, 204.05 μmol, 62% yield, formic acid salt) as a colorless solid. LCMS (ES+): m/z 856.2 [M+H]⁺

### Step 2: 2-[4-[1-(2,6-dioxo-3-piperidyl)-3-isopropyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 85)

Into a 10 mL single neck round bottom flask containing a well-stirred solution of N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-3-isopropyl-2-oxo-benzimidazol-5-yl]-1-piperidyl]acetamide (3, 160 mg, 177.39 μmol, TFA salt) in a mixture of anhydrous DCM (4 mL) and anhydrous toluene (4 mL) was added pentamethylbenzene (157.79 mg, 1.06 mmol, 172.07 μL). The reaction mixture was cooled to −78° C. and BCl₃ (1.0 M in DCM) (3.55 mL, 3.55 mmol) was added dropwise. The resulting mixture was then stirred at ambient temperature for 3 h. The reaction mixture was cooled to −78° C. and quenched with 5% MeOH in DCM (4 mL). The reaction mixture was concentrated under reduced pressure and purified by reverse-phase preparative HPLC [Purification method: Column: Zorbax C18, 7 Micron: Mobile phase A: 0.1% Formic acid in water; Mobile phase B: MeCN) to afford 2-[4-[1-(2,6-dioxo-3-piperidyl)-3-isopropyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 85, 32.17 mg, 39.53 μmol, 22% yield, formic acid salt) as an off-white solid. LCMS (ES+): m/z 766.2 [M+H]⁺

1H NMR (400 MHZ, DMSO-d6) δ 11.08 (s, 1H), 9.50 (s, 1H), 7.72-7.67 (m, 1H), 7.24 (d, J=2.5 Hz, 1H), 7.17 (s, 1H), 7.14 (dd, J=9.0, 2.5 Hz, 1H), 7.08-7.01 (m, 2H), 6.89 (d, J=8.2 Hz, 1H), 5.33 (dd, J=12.8, 5.4 Hz, 1H), 4.61 (p, J=6.9 Hz, 1H), 4.18 (t, J=5.4 Hz, 2H), 4.09 (s, 2H), 3.98 (s, 1H), 3.65-3.58 (m, 2H), 3.56-3.48 (m, 2H), 3.22-3.03 (m, 1H), 2.97-2.75 (m, 2H), 2.74-2.57 (m, 3H), 2.11-1.87 (m, 5H), 1.47 (s, 3H), 1.46 (s, 3H).

### 3-[4-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]cyclobutanecarboxamide (Example 86)

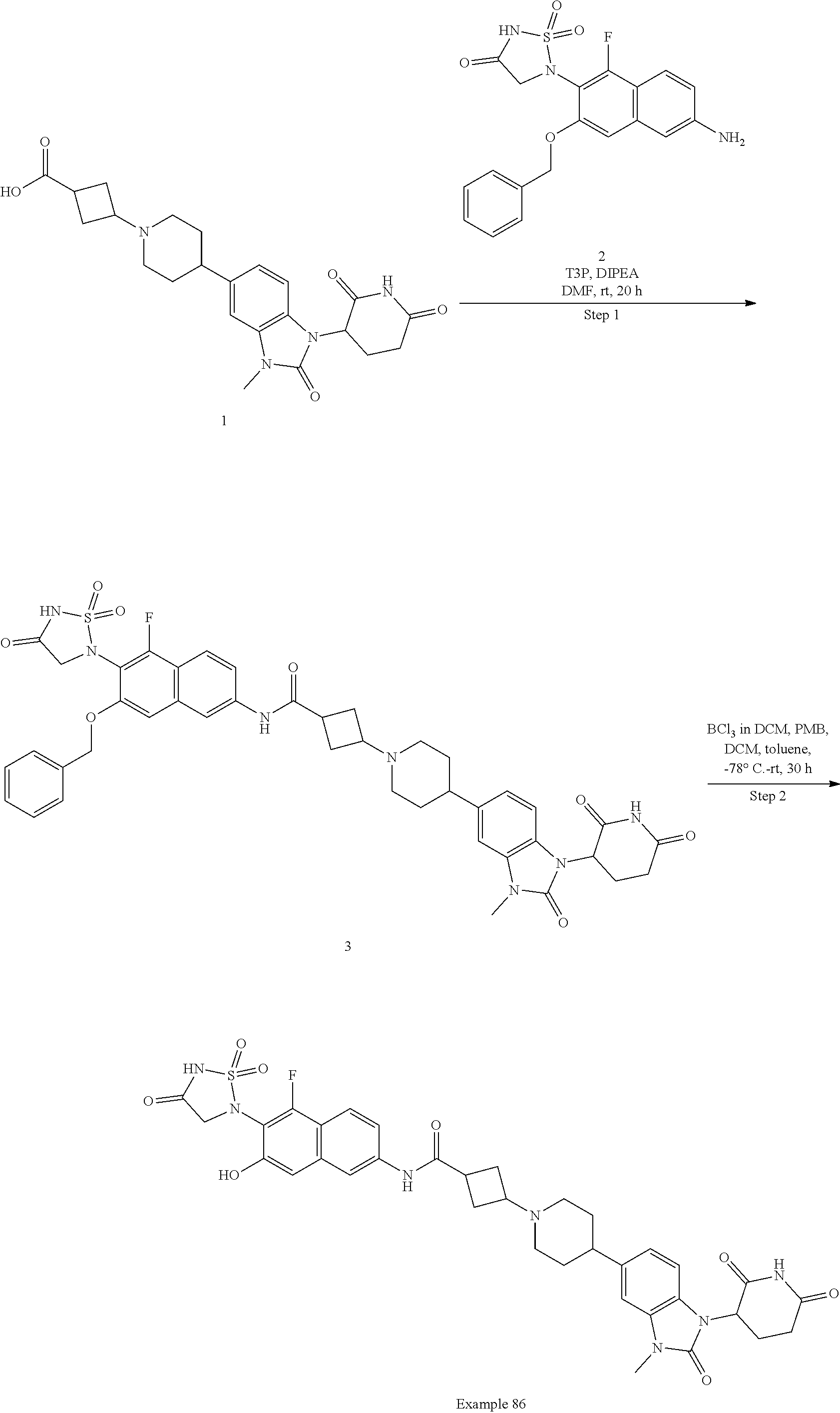 [SMILES: Cn1c(=O)n(C2CCC(=O)NC2=O)c2ccc(C3CCN(C4CC(C(=O)O)C4)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(C4CC(C(=O)Nc5ccc6c(F)c(N7CC(=O)NS7(=O)=O)c(OCc7ccccc7)cc6c5)C4)CC3)cc21 ; Cn1c(=O)n(C2CCC(=O)NC2=O)c2ccc(C3CCN(C4CC(C(=O)Nc5ccc6c(F)c(N7CC(=O)NS7(=O)=O)c(O)cc6c5)C4)CC3)cc21]

### Step 1: N-[7-benzyloxy-5-fluoro-6-(1,1,4-trioxo-1,2,5-thiadiazolidin-2-yl)-2-naphthyl]-3-[4-[1-(2,6-dioxo-3-piperidyl)-3-methyl-2-oxo-benzimidazol-5-yl]-1-piperidyl]cyclobutanecarboxamide (3)

Into a 8 mL vial containing a well-stirred solution of 3-[4-[1-(2,6-dioxo-3-piperidyl)-3-methyl-2-oxo-benzimidazol-5-yl]-1-piperidyl]cyclobutanecarboxylic acid (1, 120 mg, 201.27 μmol, HCl salt) in DMF (1 mL) was added DIPEA (407.27 mg, 3.15 mmol, 548.88 μL) followed by 1-propanephosphonic anhydride (50% in EtOAc) (200 mg, 314.28 μmol). After 10 min, 5-(6-amino-3-benzyloxy-1-fluoro-2-naphthyl)-1,1-dioxo-1,2,5-thiadiazolidin-3-one (2, 108.28 mg, 210.08 μmol, TFA salt) was added. After 20 h, the reaction mixture was concentrated under reduced pressure and purified by reverse phase preparative HPLC [Purification method: Column: Zorbax C18 (250×21.2), 5 micron, Mobile phase A: 0.1% TFA in H₂O and Mobile phase B: MeCN] to afford N-[7-benzyloxy-5-fluoro-6-(1,1,4-trioxo-1,2,5-thiadiazolidin-2-yl)-2-naphthyl]-3-[4-[1-(2,6-dioxo-3-piperidyl)-3-methyl-2-oxo-benzimidazol-5-yl]-1-piperidyl]cyclobutanecarboxamide (3, 38 mg, 39.91 μmol, 20% yield, TFA salt) as a colorless solid. LCMS (ES+): m/z 824.3 (M+H)⁺

### Step 2: 3-[4-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]cyclobutanecarboxamide (Example 86)

Into a 10 mL single neck round bottom flask containing a well-stirred solution of N-[7-benzyloxy-5-fluoro-6-(1,1,4-trioxo-1,2,5-thiadiazolidin-2-yl)-2-naphthyl]-3-[4-[1-(2,6-dioxo-3-piperidyl)-3-methyl-2-oxo-benzimidazol-5-yl]-1-piperidyl]cyclobutanecarboxamide (3, 38 mg, 42.80 μmol) in a mixture of anhydrous DCM (0.3 mL) and toluene (0.3 mL) was added pentamethylbenzene (38.07 mg, 256.81 μmol, 41.52 μL) and the reaction mixture was cooled to −78° C. Then boron trichloride (1 M in DCM) (70 mg, 597.42 μmol, 0.6 mL) was added dropwise over a period of 2 min. The reaction mixture was brought to RT and stirred for 30 h. The reaction mixture was cooled to −78° C. and quenched slowly with 10% MeOH in DCM (3 mL). The reaction mixture was allowed to attain RT and concentrated under reduced pressure at 30° C. to afford the residue, which was triturated with Et₂O (30 mL) and filtered to obtain the crude compound as a white solid, which was purified by reverse phase preparative HPLC [Purification method: Zorbax C18 (250×21.2) 7 micron; Mobile phase A: 0.1% TFA in H₂O and Mobile phase B: MeCN] to afford 3-[4-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]cyclobutanecarboxamide (Example 86, 16 mg, 18.77 μmol, 44% yield, TFA salt) as an off-white solid. LCMS (ES+): m/z 734.2 (M+H)⁺

¹H NMR (400 MHZ, DMSO-d₆) δ 11.10 (s, 1H), 10.22 (s, 1H), 9.41 (s, 1H), 8.12 (s, 1H), 7.84 (d, J=8.9 Hz, 1H), 7.48 (dd, J=9.1, 1.9 Hz, 1H), 7.11-7.05 (m, 1H), 7.03 (s, 1H), 6.95 (s, 1H), 6.93-6.89 (m, 1H), 5.36 (dd, J=12.8, 5.4 Hz, 1H), 4.07 (s, 2H), 3.71 (d, J=8.7 Hz, 1H), 3.51 (d, J=10.2 Hz, OH), 3.38-3.34 (m, 4H), 3.10-3.01 (m, 1H), 2.98-2.83 (m, 4H), 2.77-2.69 (m, 1H), 2.65-2.58 (m, 2H), 2.12-1.97 (m, 3H), 1.95-1.79 (m, 2H).

### 2-[4-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]-2-oxo-acetamide (Example 87)

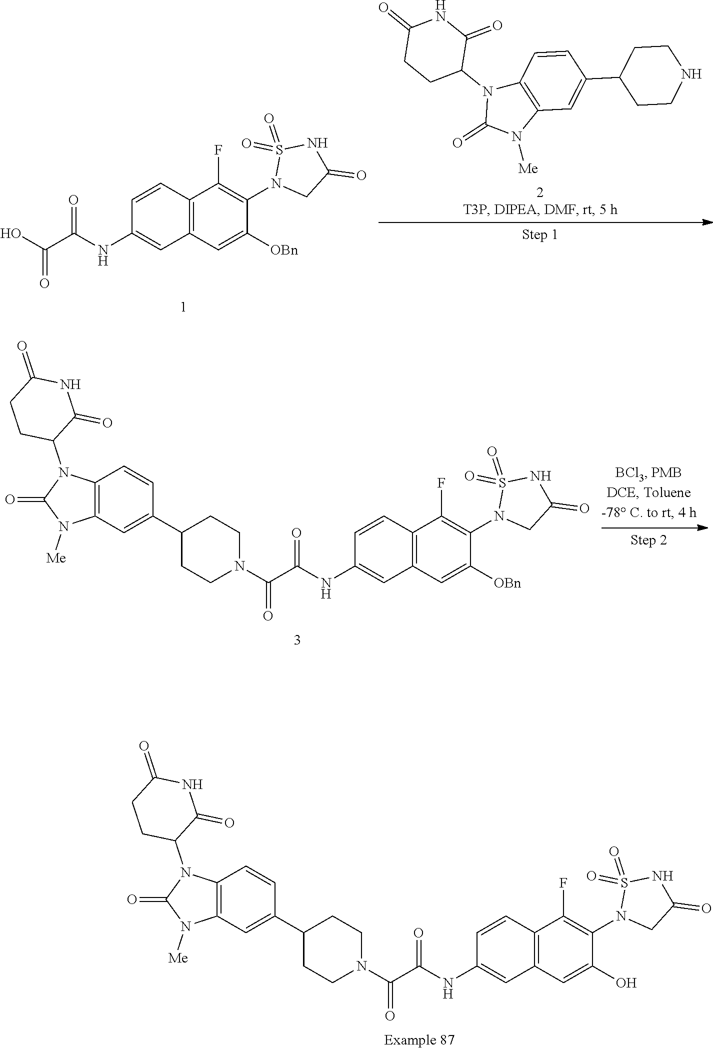 [SMILES: O=C1CN(c2c(OCc3ccccc3)cc3cc(NC(=O)C(=O)O)ccc3c2F)S(=O)(=O)N1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)C(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN(C(=O)C(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-1-piperidyl]-2-oxo-acetamide (3)

Into a 25 mL single neck round bottom flask containing a well-stirred solution of 2-[[7-benzyloxy-5-fluoro-6-(1,1,4-trioxo-1,2,5-thiadiazolidin-2-yl)-2-naphthyl]amino]-2-oxo-acetic acid (1, 300 mg, 616 μmol) in anhydrous DMF (2 mL) was added DIPEA (203.77 mg, 1.58 mmol, 274.62 μL) and propylphosphonic anhydride solution (≥50 wt. % in ethyl acetate) (401.33 mg, 630.66 μmol, 50% purity) and the resulting mixture was stirred for 10 min at RT. Then, 3-[3-methyl-2-oxo-5-(4-piperidyl)benzimidazol-1-yl]piperidine-2,6-dione (2, 199.11 mg, 525.55 μmol. HCl salt) was added to the reaction mixture and stirred at RT for 4 h. The volatiles were removed and the residue was purified by reverse phase column chromatography [Method: Silicycle 120 g (C18) 25 micron column, solvent system 0.1% formic acid in water/ACN] to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-1-piperidyl]-2-oxo-acetamide (3, 150 mg, 27% yield) as a white solid. LCMS (ES+): m/z 798.0 [M+H]⁺

### Step 2: 2-[4-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]-2-oxo-acetamide (Example 87)

Into a 10 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-1-piperidyl]-2-oxo-acetamide (3, 150 mg, 188 μmol) in a mixture of anhydrous 1,2-dichloroethane (1 mL) and anhydrous toluene (0.5 mL) was added pentamethyl benzene (74.78 mg, 504.43 μmol) under nitrogen atmosphere. The reaction mixture was then cooled to −78° C. and treated with dropwise addition of boron trichloride (1.0 M solution in DCM) (3.36 mL, 3.36 mmol). The reaction mixture was stirred at RT for 5 h. The mixture was cooled to −78° C. and quenched with 3% MeOH in DCM (2 mL). Volatiles were removed under reduced pressure and the residue was triturated with diethyl ether (10 mL×2). The solid was purified by reverse-phase column chromatography [Method: Biotage C18 (150×19) mm 5 micron column. Mobile phase A: 0.1% TFA in water. Mobile phase B: ACN] to afford 2-[4-[1-(2,6-dioxo-3-piperidyl)-3-methyl-2-oxo-benzimidazol-5-yl]-1-piperidyl]-N-[5-fluoro-7-hydroxy-6-(1,1,4-trioxo-1,2,5-thiadiazolidin-2-yl)-2-naphthyl]-2-oxo-acetamide (Example 87, 49 mg, 36% yield) as a colorless solid. LCMS (ES+): m/z 708.1 [M+H]⁺

¹H NMR (400 MHZ, DMSO-d₆) δ 11.10 (s, 1H), 11.04 (s, 1H), 10.42 (s, 1H), 8.21 (d, J=1.8 HZ, 1H), 7.90 (d, J=8.9 Hz, 1H), 7.52 (dd, J=9.1, 2.0 Hz, 1H), 7.12 (d, J=1.5 Hz, 1H), 7.07-7.00 (m, 2H), 6.98-6.91 (m, 1H), 5.35 (dd, J=12.8, 5.4 Hz, 1H), 4.51 (d, J=12.8 Hz, 1H), 4.36 (s, 2H), 3.95 (d, J=13.1 Hz, 1H), 3.34 (s, 3H), 3.32-3.22 (m, 1H), 2.96-2.83 (m, 3H), 2.76-2.56 (m, 3H), 2.07-1.95 (m, 1H), 1.95-1.82 (m, 2H), 1.80-1.60 (m, 2H).

### 3-[5-[1-[2-[3-[8-fluoro-6-hydroxy-7-(1,1,4-trioxo-1,2,5-thiadiazolidin-2-yl)-2-naphthyl]-2,5-dihydropyrrol-1-yl]-2-oxo-ethyl]-4-piperidyl]-3-methyl-2-oxo-benzimidazol-1-yl]piperidine-2,6-dione (Example 88)

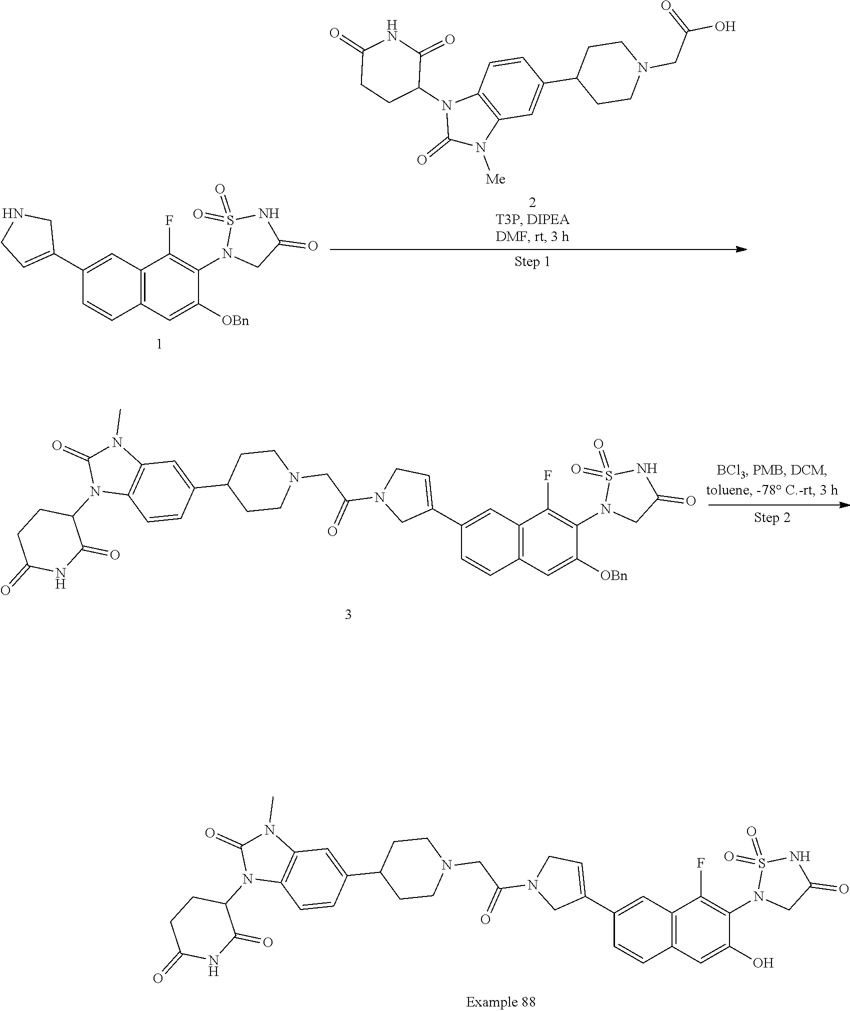 [SMILES: Cn1c(=O)n(C2CCC(=O)NC2=O)c2ccc(C3CCN(CC(=O)O)CC3)cc21 ; O=C1CN(c2c(OCc3ccccc3)cc3ccc(C4=CCNC4)cc3c2F)S(=O)(=O)N1 ; Cn1c(=O)n(C2CCC(=O)NC2=O)c2ccc(C3CCN(CC(=O)N4CC=C(c5ccc6cc(OCc7ccccc7)c(N7CC(=O)NS7(=O)=O)c(F)c6c5)C4)CC3)cc21 ; Cn1c(=O)n(C2CCC(=O)NC2=O)c2ccc(C3CCN(CC(=O)N4CC=C(c5ccc6cc(O)c(N7CC(=O)NS7(=O)=O)c(F)c6c5)C4)CC3)cc21]

### Step 1: 3-[5-[1-[2-[3-[[6-benzyloxy-8-fluoro-7-(1,1,4-trioxo-1,2,5-thiadiazolidin-2-yl)-2-naphthyl]-2,5-dihydropyrrol-1-yl]-2-oxo-ethyl]-4-piperidyl]-3-methyl-2-oxo-benzimidazol-1-yl]piperidine-2,6-dione (3)

Into a 50 mL single neck round bottom flask containing well-stirred solution of 2-[4-[1-(2,6-dioxo-3-piperidyl)-3-methyl-2-oxo-benzimidazol-5-yl]-1-piperidyl]acetic acid (2, 205.97 mg, 362.46 μmol, TFA salt), 5-[3-benzyloxy-7-(2,5-dihydro-1H-pyrrol-3-yl)-1-fluoro-2-naphthyl]-1,1-dioxo-1,2,5-thiadiazolidin-3-one (1, 220 mg, 362.46 μmol, TFA salt) in anhydrous DMF (5 mL) were added propylphosphonic anhydride solution (≥50 wt. % in ethyl acetate) (345.98 mg, 1.09 mmol, 0.4 mL) and DIPEA (742.00 mg, 5.74 mmol, 1 mL). After 3 h, the solvent was removed from the reaction mixture and water (10 mL) was added. The precipitate was filtered, washed with water and dried under reduced pressure to afford 3-[5-[1-[2-[3-[[6-benzyloxy-8-fluoro-7-(1,1,4-trioxo-1,2,5-thiadiazolidin-2-yl)-2-naphthyl]-2,5-dihydropyrrol-1-yl]-2-oxo-ethyl]-4-piperidyl]-3-methyl-2-oxo-benzimidazol-1-yl]piperidine-2,6-dione (3, 250 mg, 184.86 μmol, 51% yield, 62% purity) as an off-white solid. LCMS (ES+): m/z 836.2 [M+H]⁺

### Step 4: 3-[5-[1-[2-[3-[8-fluoro-6-hydroxy-7-(1,1,4-trioxo-1,2,5-thiadiazolidin-2-yl)-2-naphthyl]-2,5-dihydropyrrol-1-yl]-2-oxo-ethyl]-4-piperidyl]-3-methyl-2-oxo-benzimidazol-1-yl]piperidine-2,6-dione (Example 88)

Into a 50 mL single neck round bottom flask containing well-stirred solution of 3-[5-[1-[2-[3-[6-benzyloxy-8-fluoro-7-(1,1,4-trioxo-1,2,5-thiadiazolidin-2-yl)-2-naphthyl]-2,5-dihydropyrrol-1-yl]-2-oxo-ethyl]-4-piperidyl]-3-methyl-2-oxo-benzimidazol-1-yl]piperidine-2,6-dione (3, 250 mg, 184.86 μmol), pentamethylbenzene (137.02 mg, 924.31 μmol, 149.43 μL) in anhydrous DCM (3 mL) and toluene (3 mL) was added boron trichloride (1.0 M in DCM) (433.20 mg, 3.70 mmol, 3.7 mL) at −78° C. The reaction mixture was stirred at ambient temperature for 3 h. The reaction mixture was quenched with 2 mL of 5% MeOH in DCM at −78° C. The solvents were removed under reduced pressure and the residue washed with diethyl ether (20 mL). The material was purified by reverse phase prep HPLC [Purification method: Column: X-Select C18 (150×19) 5 micron, mobile phase: 0.1% TFA in water and MeCN] to afford 3-[5-[1-[2-[3-[8-fluoro-6-hydroxy-7-(1,1,4-trioxo-1,2,5-thiadiazolidin-2-yl)-2-naphthyl]-2,5-dihydropyrrol-1-yl]-2-oxo-ethyl]-4-piperidyl]-3-methyl-2-oxo-benzimidazol-1-yl]piperidine-2,6-dione (Example 88, 50 mg, 57.70 μmol, 31% yield, TFA salt) as a colorless solid. LCMS (ES+): m/z 746.2 [M+H]⁺

¹H NMR (400 MHZ, DMSO-d₆) δ 11.11 (s, 1H), 10.39 (s, 1H), 9.71 (s, 1H), 7.87-7.67 (m, 3H), 7.14-7.05 (m, 3H), 6.95 (d, J=8.2 Hz, 1H), 6.67-6.60 (m, 1H), 5.34 (dd, J=12.9, 5.4 Hz, 1H), 4.86-4.77 (m, 1H), 4.70 (s, 1H), 4.50 (s, 1H), 4.45-4.39 (m, 1H), 4.36 (s, 1H), 4.28 (s, 1H), 4.23 (d, J=2.9 Hz, 2H), 3.68-3.59 (m, 4H), 3.35 (s, 3H), 3.25-3.13 (m, 2H), 2.98-2.82 (m, 2H), 2.77-2.58 (m, 2H), 2.17-1.92 (m, 5H).

### 3-[5-[1-[3-[3-[8-fluoro-6-hydroxy-7-(1,1,4-trioxo-1,2,5-thiadiazolidin-2-yl)-2-naphthyl]-2,5-dihydropyrrol-1-yl]-3-oxo-propyl]-4-piperidyl]-3-methyl-2-oxo-benzimidazol-1-yl]piperidine-2,6-dione (Example 89)

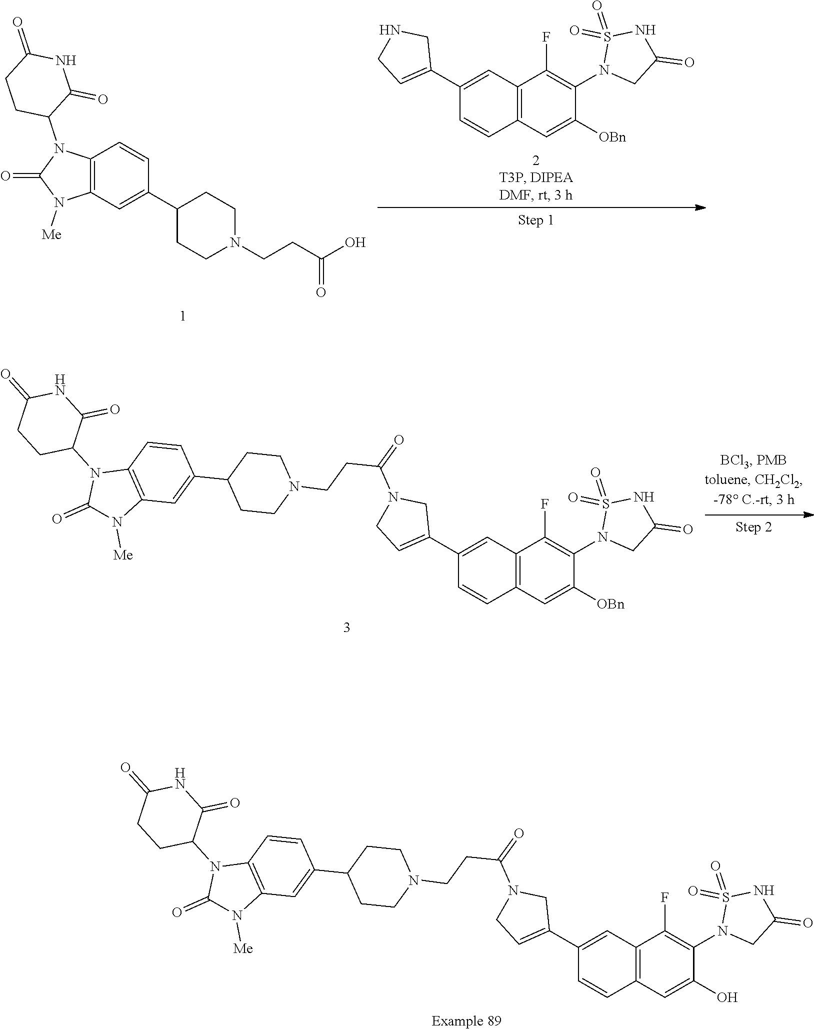 [SMILES: Cn1c(=O)n(C2CCC(=O)NC2=O)c2ccc(C3CCN(CCC(=O)O)CC3)cc21 ; O=C1CN(c2c(OCc3ccccc3)cc3ccc(C4=CCNC4)cc3c2F)S(=O)(=O)N1 ; Cn1c(=O)n(C2CCC(=O)NC2=O)c2ccc(C3CCN(CCC(=O)N4CC=C(c5ccc6cc(OCc7ccccc7)c(N7CC(=O)NS7(=O)=O)c(F)c6c5)C4)CC3)cc21 ; Cn1c(=O)n(C2CCC(=O)NC2=O)c2ccc(C3CCN(CCC(=O)N4CC=C(c5ccc6cc(O)c(N7CC(=O)NS7(=O)=O)c(F)c6c5)C4)CC3)cc21]

### Step 1: 3-[5-[1-[3-[3-[[6-benzyloxy-8-fluoro-7-(1,1,4-trioxo-1,2,5-thiadiazolidin-2-yl)-2-naphthyl]-2,5-dihydropyrrol-1-yl]-3-oxo-propyl]-4-piperidyl]-3-methyl-2-oxo-benzimidazol-1-yl]piperidine-2,6-dione (3)

Into a 50) mL single neck round bottom flask containing a well-stirred solution of 3-[4-[1-(2,6-dioxo-3-piperidyl)-3-methyl-2-oxo-benzimidazol-5-yl]-1-piperidyl]propanoic acid (1, 350 mg, 662.28 μmol, TFA salt) and 5-[3-benzyloxy-7-(2,5-dihydro-1H-pyrrol-3-yl)-1-fluoro-2-naphthyl]-1,1-dioxo-1,2,5-thiadiazolidin-3-one (2, 375.85 mg, 662.28 μmol, TFA salt) in anhydrous DMF (5 mL) were added DIPEA (427.97 mg, 3.31 mmol, 576.77 μL) and 1-propylphosphonic anhydride (50% in EtOAc) (842.9 mg, 1.32 mmol). After 3 h. the reaction mixture was concentrated under reduced pressure and the residue poured in cold water (50) mL). The precipitate was filtered and dried to obtain 3-[5-[1-[3-[3-[[6-benzyloxy-8-fluoro-7-(1,1,4-trioxo-1,2,5-thiadiazolidin-2-yl)-2-naphthyl]-2,5-dihydropyrrol-1-yl]-3-oxo-propyl]-4-piperidyl]-3-methyl-2-oxo-benzimidazol-1-yl]piperidine-2,6-dione (3, 250 mg, 242.43 μmol, 37% yield) as an off-white solid. LCMS (ES+): m/z 850.3 [M+H]⁺

### Step 2: 3-[5-[1-[3-[3-[8-fluoro-6-hydroxy-7-(1,1,4-trioxo-1,2,5-thiadiazolidin-2-yl)-2-naphthyl]-2,5-dihydropyrrol-1-yl]-3-oxo-propyl]-4-piperidyl]-3-methyl-2-oxo-benzimidazol-1-yl]piperidine-2,6-dione (Example 89)

Into a 25 mL single neck round bottom flask containing a well-stirred solution of 3-[5-[1-[3-[3-[6-benzyloxy-8-fluoro-7-(1,1,4-trioxo-1,2,5-thiadiazolidin-2-yl)-2-naphthyl]-2,5-dihydropyrrol-1-yl]-3-oxo-propyl]-4-piperidyl]-3-methyl-2-oxo-benzimidazol-1-yl]piperidine-2,6-dione (3, 100 mg, 117.66 μmol) in a 1:1 mixture of anhydrous toluene (3 mL) and anhydrous DCM (3 mL) was added pentamethylbenzene (87.21 mg, 588.29 μmol) at ambient temperature under nitrogen atmosphere. The reaction mixture was cooled to −78° C. and a BCl₃ (1.0 M in in DCM) (1.76 mL, 1.76 mmol) was added dropwise and the resulting mixture was stirred at ambient temperature for 3 h. The reaction mixture was cooled to −78° C. and quenched with 5% MeOH in DCM (2 mL). The reaction mixture was concentrated under reduced pressure and purified by reverse-phase preparative HPLC [Purification method: Column: X bridge (150)×19) mm, 5 micron; Mobile phase A: 0.1% TFA in MQ-water; Mobile phase B: Acetonitrile] to afford 3-[5-[1-[3-[3-[8-fluoro-6-hydroxy-7-(1,1,4-trioxo-1,2,5-thiadiazolidin-2-yl)-2-naphthyl]-2,5-dihydropyrrol-1-yl]-3-oxo-propyl]-4-piperidyl]-3-methyl-2-oxo-benzimidazol-1-yl]piperidine-2,6-dione (Example 89, 40 mg, 45.58 μmol, 39% yield, TFA salt) as an off-white solid. LCMS (ESI): m/z 760.2 [M+H]^{+ 1}H NMR (400 MHZ, DMSO-d₆) δ 11.11 (s, 1H), 10.21 (s, 1H), 9.06 (s, 1H), 7.87-7.76 (m, 2H), 7.73 (d, J=7.9 Hz, 1H), 7.13-7.04 (m, 3H), 6.95-6.90 (m, 1H), 6.65-6.61 (m, 1H), 5.36 (dd, J=12.8, 5.4 Hz, 1H), 4.89-4.82 (m, 1H), 4.64 (s, 1H), 4.55 (s, 1H), 4.36 (s, 1H), 4.21 (d, J=5.4 Hz, 2H), 3.48-3.41 (m, 2H), 3.35 (d, J=1.1 Hz, 3H), 3.20-3.07 (m, 2H), 3.03-2.84 (m, 4H), 2.77-2.58 (m, 2H), 2.10-1.90 (m, 5H).

### 3-[5-[1-[2-[3-[8-fluoro-6-hydroxy-7-(1,1,4-trioxo-1,2,5-thiadiazolidin-2-yl)-2-naphthyl]pyrrolidin-1-yl]-2-oxo-ethyl]-4-piperidyl]-3-methyl-2-oxo-benzimidazol-1-yl]piperidine-2,6-dione (Example 90)

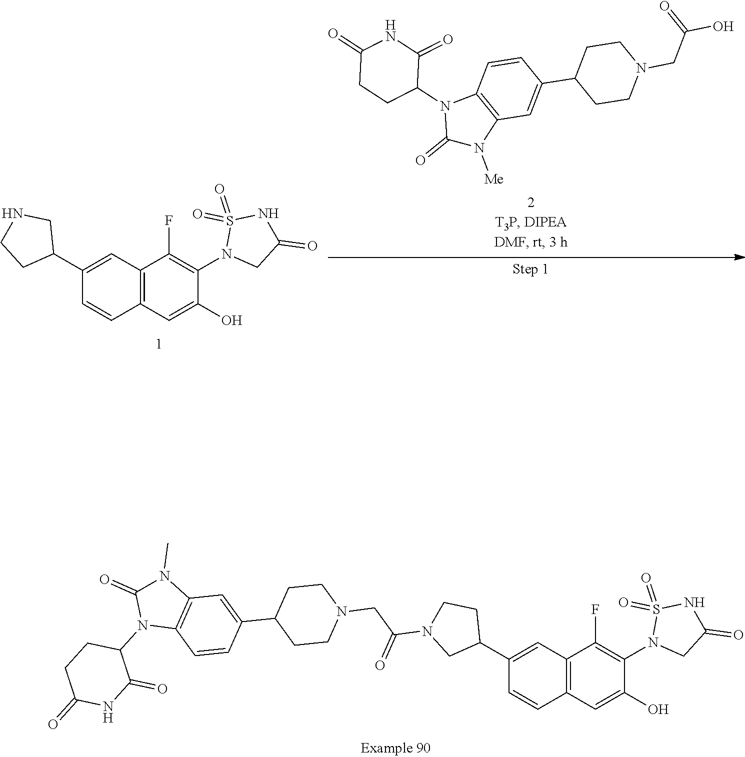 [SMILES: Cn1c(=O)n(C2CCC(=O)NC2=O)c2ccc(C3CCN(CC(=O)O)CC3)cc21 ; O=C1CN(c2c(O)cc3ccc(C4CCNC4)cc3c2F)S(=O)(=O)N1 ; Cn1c(=O)n(C2CCC(=O)NC2=O)c2ccc(C3CCN(CC(=O)N4CCC(c5ccc6cc(O)c(N7CC(=O)NS7(=O)=O)c(F)c6c5)C4)CC3)cc21]

### Step 1: 3-[5-[1-[2-[3-[8-fluoro-6-hydroxy-7-(1,1,4-trioxo-1,2,5-thiadiazolidin-2-yl)-2-naphthyl]pyrrolidin-1-yl]-2-oxo-ethyl]-4-piperidyl]-3-methyl-2-oxo-benzimidazol-1-yl]piperidine-2,6-dione (Example 90)

Into a 10 mL single neck round bottom flask containing well-stirred solution of 2-[4-[1-(2,6-dioxo-3-piperidyl)-3-methyl-2-oxo-benzimidazol-5-yl]-1-piperidyl]acetic acid (2, 100 mg, 175.97 μmol, TFA salt) in anhydrous DMF (2 mL) were added propylphosphonic anhydride solution (≥50 wt. % in ethyl acetate) (223.97 mg, 351.95 μmol, 0.23 mL), DIPEA (113.72 mg, 879.87 μmol, 153.26 μL) and 5-(1-fluoro-3-hydroxy-7-pyrrolidin-3-yl-2-naphthyl)-1,1-dioxo-1,2,5-thiadiazolidin-3-one (1, 97.37 mg, 175.97 μmol, TFA salt). The reaction mixture was stirred at RT for 3 h. The reaction mixture was quenched with water and the precipitate was filtered and dried under reduced pressure. The material was purified by reverse phase prep HPLC [Purification method: Column: X-Bridge C18 (19×150) 5 micron, Mobile phase A: 0.1% TFA in water; Mobile phase B: MeCN] to afford 3-[5-[1-[2-[3-[8-fluoro-6-hydroxy-7-(1,1,4-trioxo-1,2,5-thiadiazolidin-2-yl)-2-naphthyl]pyrrolidin-1-yl]-2-oxo-ethyl]-4-piperidyl]-3-methyl-2-oxo-benzimidazol-1-yl]piperidine-2,6-dione (Example 90, 65 mg, 74.79 μmol, 43% yield, TFA salt) as a colorless solid. LCMS (ES+): m/z 748.2 [M+H]⁺

¹H NMR (400 MHZ, DMSO-d6): δ 11.11 (s, 1H), 10.07 (s, 1H), 9.62 (s, 1H), 7.81-7.75 (m, 2H), 7.50-7.48 (m, 1H), 7.14-7.04 (m, 3H), 6.95 (d, J=8.00 Hz, 1H), 5.38-5.33 (m, 1H), 4.29-4.22 (m, 4H), 3.98-3.96 (m, 2H), 3.76-3.45 (m, 5H), 3.33 (s, 3H), 3.20-3.05 (m, 2H), 2.91-2.87 (m, 2H), 2.73-2.61 (m, 2H), 2.14-2.00 (m, 6H).

### 3-[5-[1-[3-[4-[8-fluoro-6-hydroxy-7-(1,1,4-trioxo-1,2,5-thiadiazolidin-2-yl)-2-naphthyl]pyrazol-1-yl]propyl]-4-piperidyl]-3-methyl-2-oxo-benzimidazol-1-yl]piperidine-2,6-dione (Example 91)

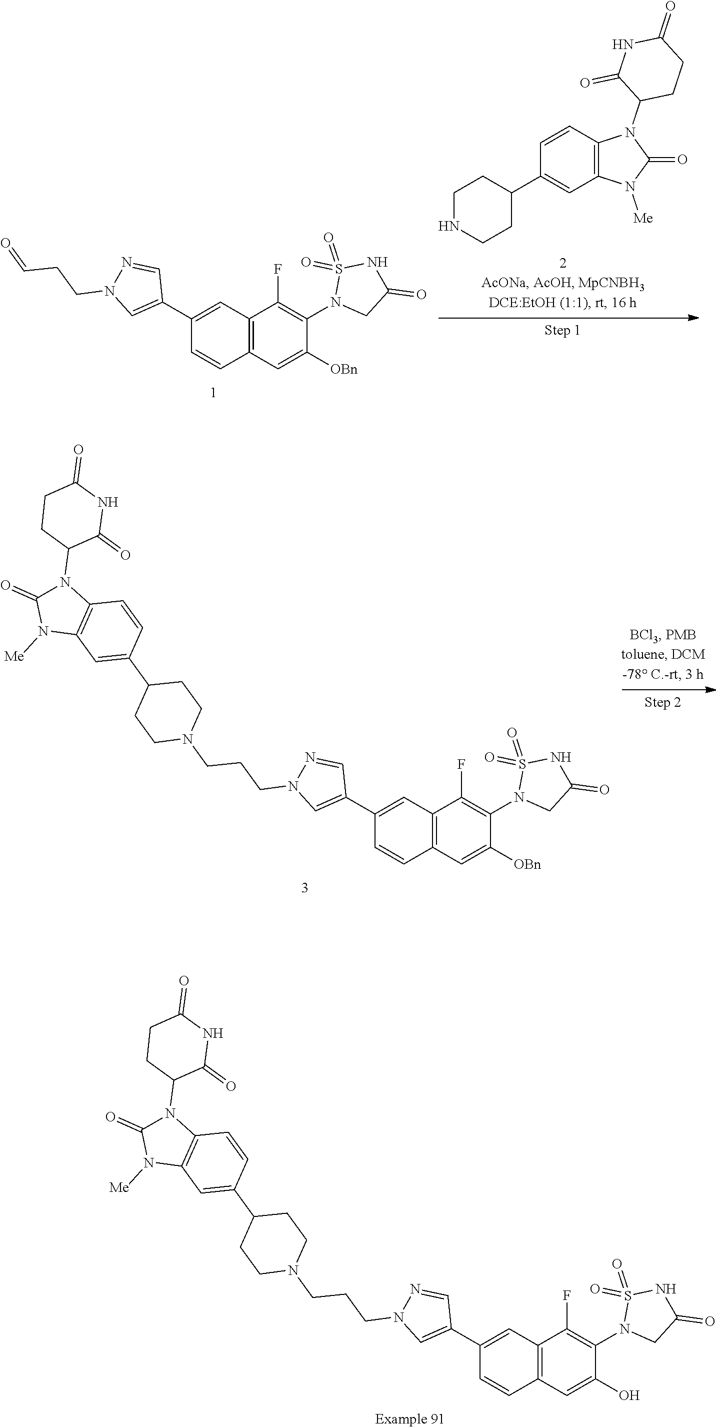 [SMILES: O=CCCn1cc(-c2ccc3cc(OCc4ccccc4)c(N4CC(=O)NS4(=O)=O)c(F)c3c2)cn1 ; Cn1c(=O)n(C2CCC(=O)NC2=O)c2ccc(C3CCNCC3)cc21 ; Cn1c(=O)n(C2CCC(=O)NC2=O)c2ccc(C3CCN(CCCn4cc(-c5ccc6cc(OCc7ccccc7)c(N7CC(=O)NS7(=O)=O)c(F)c6c5)cn4)CC3)cc21 ; Cn1c(=O)n(C2CCC(=O)NC2=O)c2ccc(C3CCN(CCCn4cc(-c5ccc6cc(O)c(N7CC(=O)NS7(=O)=O)c(F)c6c5)cn4)CC3)cc21]

### Step 1: 3-[5-[1-[3-[4-[6-benzyloxy-8-fluoro-7-(1,1,4-trioxo-1,2,5-thiadiazolidin-2-yl)-2-naphthyl]pyrazol-1-yl]propyl]-4-piperidyl]-3-methyl-2-oxo-benzimidazol-1-yl]piperidine-2,6-dione (3)

Into a 25 mL single neck round bottom flask containing a stirred solution of 3-[3-methyl-2-oxo-5-(4-piperidyl)benzimidazol-1-yl]piperidine-2,6-dione (2, 200 mg, 527.91 μmol, HCl salt) and 3-[4-[6-benzyloxy-8-fluoro-7-(1,1,4-trioxo-1,2,5-thiadiazolidin-2-yl)-2-naphthyl]pyrazol-1-yl]propanal (1, 241.61 mg, 475.12 μmol) in anhydrous DCE (3.0 mL) and ethanol (3.0 mL) was added sodium acetate (129.91 mg, 1.58 mmol). After 1 h, MP-cyanoborohydride (396 mg, 791.87 μmol) was added and the suspension was stirred at RT for 16 h. The reaction mixture was filtered and concentrated. The residue was purified by reverse phase column chromatography [C18-column, 120 g, Mobile phase A: 0.1% TFA in water; Mobile phase B: Acetonitrile] to obtain 3-[5-[1-[3-[4-[6-benzyloxy-8-fluoro-7-(1,1,4-trioxo-1,2,5-thiadiazolidin-2-yl)-2-naphthyl]pyrazol-1-yl]propyl]-4-piperidyl]-3-methyl-2-oxo-benzimidazol-1-yl]piperidine-2,6-dione (3, 200 mg, 34% yield). LCMS (ES+): m/z 835.2 [M+H]⁺

### Step 2: 3-[5-[1-[3-[4-[8-fluoro-6-hydroxy-7-(1,1,4-trioxo-1,2,5-thiadiazolidin-2-yl)-2-naphthyl]pyrazol-1-yl]propyl]-4-piperidyl]-3-methyl-2-oxo-benzimidazol-1-yl]piperidine-2,6-dione (Example 91)

Into a 25 mL single neck round bottom flask containing a well-stirred solution of 3-[5-[1-[3-[4-[6-benzyloxy-8-fluoro-7-(1,1,4-trioxo-1,2,5-thiadiazolidin-2-yl)-2-naphthyl]pyrazol-1-yl]propyl]-4-piperidyl]-3-methyl-2-oxo-benzimidazol-1-yl]piperidine-2,6-dione (3, 200 mg, 239.55 μmol) in anhydrous DCM (1.0 mL) and toluene (1.0 mL) was added pentamethyl benzene (177.56 mg, 1.20 mmol). The reaction mixture was cooled to −78° C., then boron trichloride (1.0 M in DCM) (561.35 mg, 4.79 mmol, 4.79 mL) was added. The reaction mixture was stirred at ambient temperature for 3 h. The reaction was quenched with 10 mL of 5% MeOH in DCM at −78° C. The volatiles were removed under reduced pressure. The residue was triturated with tert-butylmethyl ether (10 mL) and decanted and purified by reverse phase prep HPLC [Method: X-Bridge C18 (19×150) 5 micron; Mobile phase A: 0.1% TFA in water; Mobile phase B: Acetonitrile] to afford 3-[5-[1-[3-[4-[8-fluoro-6-hydroxy-7-(1,1,4-trioxo-1,2,5-thiadiazolidin-2-yl)-2-naphthyl]pyrazol-1-yl]propyl]-4-piperidyl]-3-methyl-2-oxo-benzimidazol-1-yl]piperidine-2,6-dione (Example 91, 10 mg, 5% yield, TFA salt) as an off-white solid. LCMS (ES+): m/z 745.2 [M+H]⁺

¹H NMR (400 MHZ, DMSO-d₆): δ 11.11 (s, 1H), 9.90 (s, 1H), 9.12 (s, 1H), 8.40 (s, 1H), 8.11 (s, 1H), 8.04 (s, 1H), 7.79-7.74 (m, 2H), 7.08-7.04 (m, 3H), 6.91 (d, J=8.40 Hz, 1H), 5.36 (dd, J=6.00, 12.40 Hz, 1H), 4.30-4.27 (m, 2H), 4.17 (s, 2H), 3.64-3.61 (m, 2H), 3.14-3.08 (m, 4H), 2.91-2.87 (m, 2H), 2.71-2.68 (m, 1H), 2.65-2.60 (m, 1H), 2.31-2.29 (m, 2H), 2.07-1.99 (m, 3H), 1.92-1.86 (m, 2H).

### 2-[4-[1-[(3R)-2,6-dioxo-3-piperidyl]-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 92)

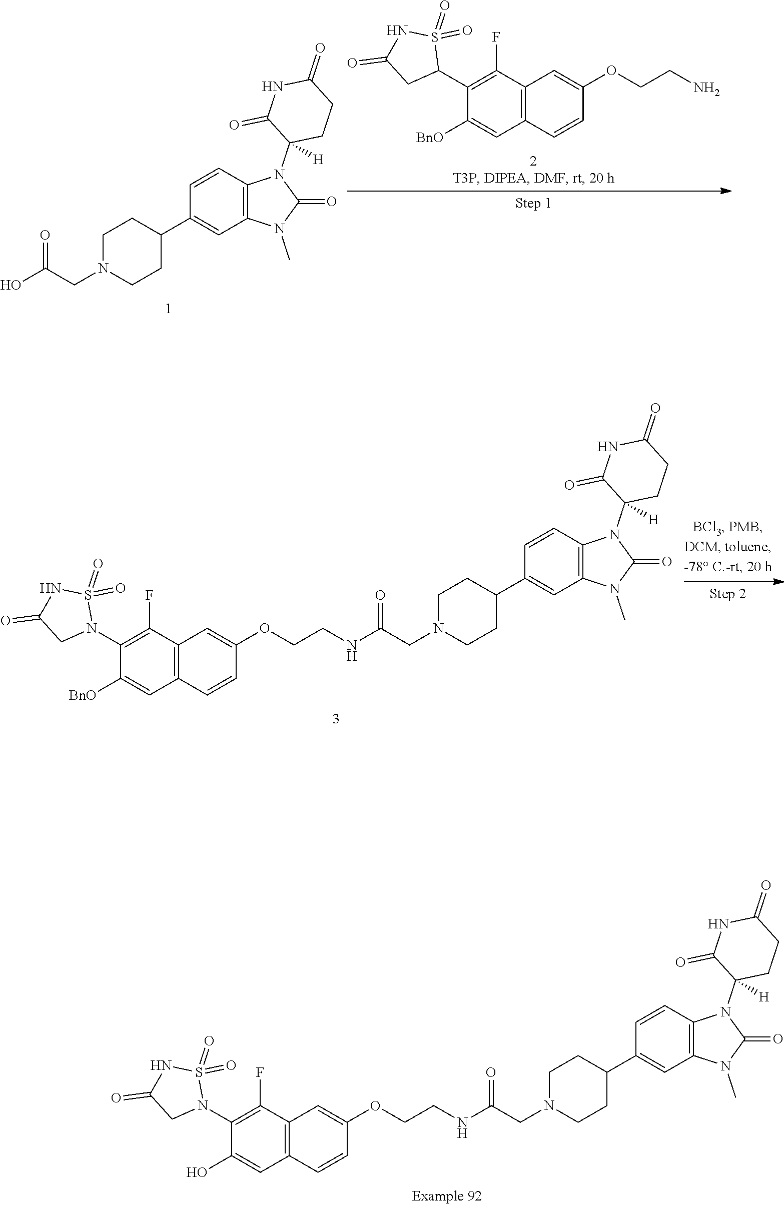

### Step 1: N-[2-[[6-benzyloxy-8-fluoro-7-(1,1,4-trioxo-1,2,5-thiadiazolidin-2-yl)-2-naphthyl]oxy]ethyl]-2-[4-[1-[(3R)-2,6-dioxo-3-piperidyl]-3-methyl-2-oxo-benzimidazol-5-yl]-1-piperidyl]acetamide (3)

Into a 8 mL vial containing a well-stirred solution of 2-[4-[1-[(3R)-2,6-dioxo-3-piperidyl]-3-methyl-2-oxo-benzimidazol-5-yl]-1-piperidyl]acetic acid (1, 120 mg, 212.50 μmol-TFA salt) in DMF (2 mL) was added DIPEA (274.63 mg, 2.12 mmol, 370.13 μL) followed by 1-propanephosphonic anhydride (50% in ethyl acetate) (202.84 mg, 318.75 μmol, purity 50%). After 10 min. 5-[7-(2-aminoethoxy)-3-benzyloxy-1-fluoro-2-naphthyl]-1,1-dioxo-1,2,5-thiadiazolidin-3-one (2, 126.46 mg, 212.50 μmol, TFA salt) was added. After 20 h. the reaction mixture was concentrated under reduced pressure and purified via reverse phase preparative HPLC [Purification method: Column: X-select-C18 (19 mm×150 mm) 5 micron. Mobile phase A: 0.1% TFA in H₂O and Mobile phase B: MeCN] to afford N-[2-[[6-benzyloxy-8-fluoro-7-(1,1,4-trioxo-1,2,5-thiadiazolidin-2-yl)-2-naphthyl]oxy]ethyl]-2-[4-[1-[(3R)-2,6-dioxo-3-piperidyl]-3-methyl-2-oxo-benzimidazol-5-yl]-1-piperidyl]acetamide (3, 68 mg, 71.90 μmol, 34% yield, TFA salt) as a colorless solid. LCMS (ES+): m/z 828.2 [M+H]⁺

### Step 2: 2-[4-[1-[(3R)-2,6-dioxo-3-piperidyl]-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 92)

Into a 10 mL single neck round bottom flask containing a well-stirred solution of N-[2-[[6-benzyloxy-8-fluoro-7-(1,1,4-trioxo-1,2,5-thiadiazolidin-2-yl)-2-naphthyl]oxy]ethyl]-2-[4-[1-[(3R)-2,6-dioxo-3-piperidyl]-3-methyl-2-oxo-benzimidazol-5-yl]-1-piperidyl]acetamide (3, 60 mg, 63.44 μmol, TFA salt) in a mixture of anhydrous toluene (1.5 mL) and DCM (1.5 mL) was added pentamethylbenzene (56.43 mg, 380.64 μmol, 61.54 μL) and the reaction mixture was cooled to −78° C. Then boron trichloride (1.0 M in DCM) (234.4 mg, 2.00 mmol, 2.0 mL) was added dropwise over a period of 2 min. The reaction mixture was brought to RT. After 20 h. the reaction mixture was cooled to −78° C. and quenched with 10% MeOH in DCM (3 mL). The reaction mixture was concentrated under reduced pressure at 30° C. and the residue was washed with Et₂O (30 mL) and filtered. The material was purified by reverse phase preparative HPLC [Purification method: X-select-C18 (19 mm×150 mm) 5 micron; Mobile phase A: 0.1% TFA in water and Mobile phase B: MeCN] to afford 2-[4-[1-[(3R)-2,6-dioxo-3-piperidyl]-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 92, 18.9 mg, 21.26 μmol, 34% yield, TFA salt) as a colorless solid. LCMS (ES+): m/z 738.2 [M+H]⁺

¹H NMR (400 MHZ, DMSO-d6): δ 11.10 (s, 1H), 9.66 (s, 1H), 9.52 (s, 1H), 8.88 (s, 1H), 7.70 (d, J=8.80 Hz, 1H), 7.25 (d, J=2.40 Hz, 1H), 7.15 (dd, J=2.40, 9.20 Hz, 1H), 7.08-7.05 (m, 3H), 6.92 (d, J=8.00 Hz, 1H), 5.38-5.34 (m, 1H), 4.20 (t, J=5.20 Hz, 2H), 4.10 (s, 2H), 3.99 (s, 2H), 3.63-3.54 (m, 4H), 3.28-3.15 (m, 2H), 2.87-3.00 (m, 2H), 2.69-2.61 (m, 2H), 2.07-1.99 (m, 6H).

### 2-[4-[1-[(3S)-2,6-dioxo-3-piperidyl]-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 93)

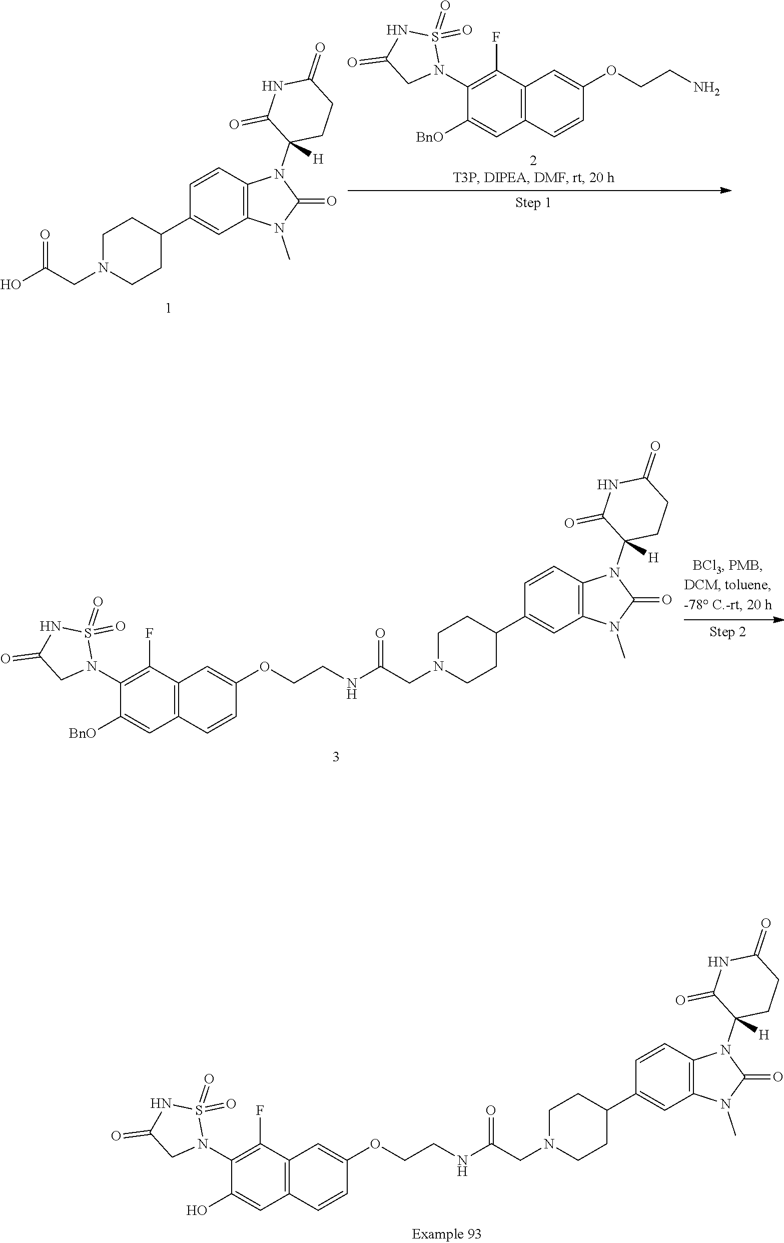

2-[4-[1-[(3S)-2,6-dioxo-3-piperidyl]-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 93) was prepared from 2-[4-[1-[(3S)-2,6-dioxo-3-piperidyl]-3-methyl-2-oxo-benzimidazol-5-yl]-1-piperidyl]acetic acid (1) and 5-[7-(2-aminoethoxy)-3-benzyloxy-1-fluoro-2-naphthyl]-1,1-dioxo-1,2,5-thiadiazolidin-3-one (2) over two steps following the same procedure as 2-[4-[1-[(3R)-2,6-dioxo-3-piperidyl]-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 92). LCMS (ES+): m/z 738.2 [M+H]⁺

¹H NMR (400 MHZ, DMSO-d₆) δ 11.10 (s, 1H), 9.65 (s, 1H), 9.53 (s, 1H), 8.87 (s, 1H), 7.69 (dd, J=9.0, 1.5 Hz, 1H), 7.24 (d, J=2.5 Hz, 1H), 7.14 (dd, J=9.0, 2.5 Hz, 1H), 7.08-7.02 (m, 3H), 6.91 (d, J=8.2 Hz, 1H), 5.35 (dd, J=12.7, 5.4 Hz, 1H), 4.18 (t, J=5.4 Hz, 2H), 4.09 (s, 2H), 3.97 (s, 2H), 3.65-3.57 (m, 2H), 3.58-3.50 (m, 2H), 3.20-3.07 (m, 2H), 2.97-2.76 (m, 2H), 2.76-2.57 (m, 2H), 2.11-1.86 (m, 5H).

### 2-[4-[4-[(2,6-dioxo-3-piperidyl)-methyl-amino]phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 94)

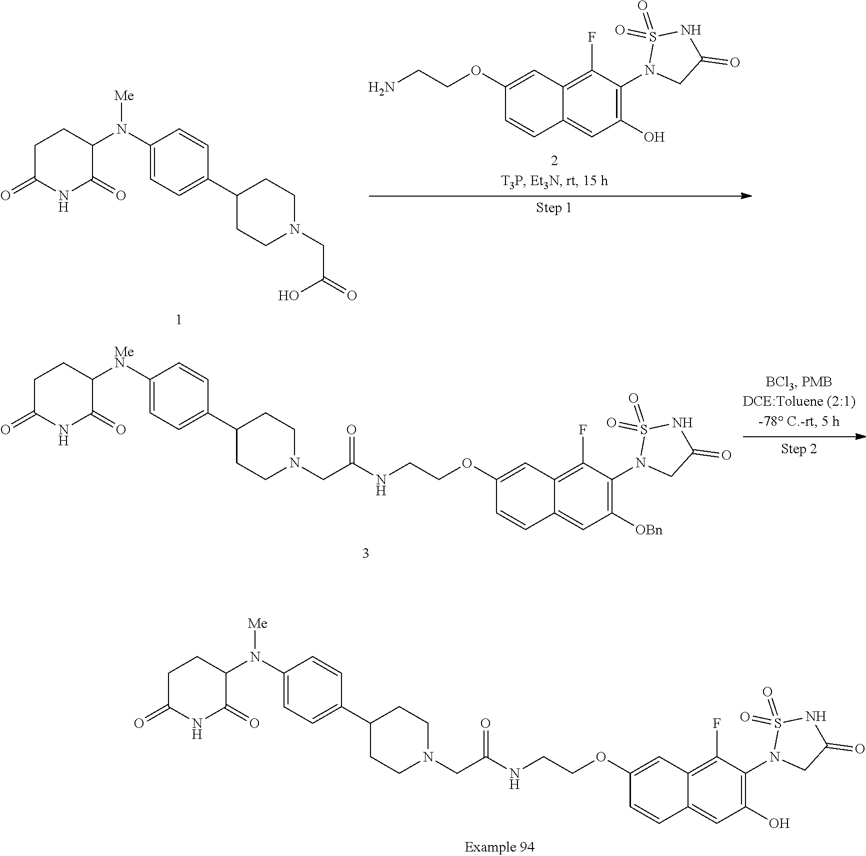 [SMILES: CN(c1ccc(C2CCN(CC(=O)O)CC2)cc1)C1CCC(=O)NC1=O ; NCCOc1ccc2cc(O)c(N3CC(=O)NS3(=O)=O)c(F)c2c1 ; CN(c1ccc(C2CCN(CC(=O)NCCOc3ccc4cc(OCc5ccccc5)c(N5CC(=O)NS5(=O)=O)c(F)c4c3)CC2)cc1)C1CCC(=O)NC1=O ; CN(c1ccc(C2CCN(CC(=O)NCCOc3ccc4cc(O)c(N5CC(=O)NS5(=O)=O)c(F)c4c3)CC2)cc1)C1CCC(=O)NC1=O]

### Step 1: N-[2-[[6-benzyloxy-8-fluoro-7-(1,1,4-trioxo-1,2,5-thiadiazolidin-2-yl)-2-naphthyl]oxy]ethyl]-2-[4-[4-[(2,6-dioxo-3-piperidyl)-methyl-amino]phenyl]-1-piperidyl]acetamide (3)

Into a 25 mL single neck round bottom flask containing a well-stirred solution of 2-[4-[4-[(2,6-dioxo-3-piperidyl)-methyl-amino]phenyl]-1-piperidyl]acetic acid (1, 100 mg, 219.29 μmol, TFA salt) in anhydrous DMF (3 mL) were added propanephosphonic acid anhydride (50 wt. % in ethyl acetate) (208.21 mg, 327.20 μmol, 50% purity) and triethylamine (82.77 mg, 817.99 μmol, 114.01 μL). After 10 min, 5-[7-(2-aminoethoxy)-3-benzyloxy-1-fluoro-2-naphthyl]-1,1-dioxo-1,2,5-thiadiazolidin-3-one (2, 127.85 mg, 272.66 μmol) was added and stirred for 15 h. The reaction mixture was concentrated under reduced pressure. The residue was purified by reverse phase prep HPLC [Method: Biotage C18 (150×19) mm, 5 micron column; Mobile phase A: 0.1% TFA in water; Mobile phase B: MeCN] to afford N-[2-[[6-benzyloxy-8-fluoro-7-(1,1,4-trioxo-1,2,5-thiadiazolidin-2-yl)-2-naphthyl]oxy]ethyl]-2-[4-[4-[(2,6-dioxo-3-piperidyl)-methyl-amino]phenyl]-1-piperidyl]acetamide (3, 90 mg, 39% yield, TFA salt). LCMS (ES+): m/z 787.2 [M+H]⁺

### Step 2: 2-[4-[4-[(2,6-dioxo-3-piperidyl)-methyl-amino]phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 94)

Into a 25 mL single neck round bottom flask containing a well-stirred solution of N-[2-[[6-benzyloxy-8-fluoro-7-(1,1,4-trioxo-1,2,5-thiadiazolidin-2-yl)-2-naphthyl]oxy]ethyl]-2-[4-[4-[(2,6-dioxo-3-piperidyl)-methyl-amino]phenyl]-1-piperidyl]acetamide (3, 90 mg, 84.92 μmol, TFA salt) in a mixture of anhydrous toluene (5 mL) and 1,2-dichloroethane (8 mL) was added pentamethyl benzene (25.18 mg, 169.83 μmol). The reaction mixture was cooled to −78° C., then boron trichloride (1.0 M in DCM) (1.7 mL, 1.70 mmol) was added dropwise. The reaction mixture was stirred at RT for 5 h. The reaction was quenched with 2 mL of 3% MeOH in DCM at −78° C. The volatiles were removed under reduced pressure and the residue was triturated with diethyl ether (10 mL×2). The material was purified by reverse phase prep HPLC [Purification method: Biotage C18 (19×150) mm, 5 micron column; Mobile phase A: 0.1% TFA in water; Mobile phase B: MeCN] to afford 2-[4-[4-[(2,6-dioxo-3-piperidyl)-methyl-amino]phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 94, 20 mg, 27% yield, TFA salt) as a colorless solid. LCMS (ES+): m/z 697.2 [M+H]^{+ 1}H NMR (400 MHZ, DMSO-d₆): δ 10.79 (s, 1H), 9.65 (bs, 1H), 9.51 (s, 1H), 8.87 (bs, 1H), 7.70 (d, J=8.40 Hz, 1H), 7.24-7.21 (m, 1H), 7.16-7.14 (m, 1H), 7.14-7.03 (m, 3H), 6.79 (d, J=8.80 Hz, 2H), 4.85 (dd, J=5.20, 12.80 Hz, 1H), 4.20-4.18 (m, 2H), 4.10 (s, 2H), 4.00-3.92 (m, 2H), 3.62-3.61 (m, 2H), 3.57-3.49 (m, 2H), 3.17-3.05 (m, 2H), 2.86-2.81 (m, 1H), 2.73 (s, 3H), 1.97-1.85 (m, 5H).

### 3-[5-[1-[5-[[8-fluoro-6-hydroxy-7-(1,1,4-trioxo-1,2,5-thiadiazolidin-2-yl)-2-naphthyl]oxy]pentyl]-4-piperidyl]-3-methyl-2-oxo-benzimidazol-1-yl]piperidine-2,6-dione (Example 95)

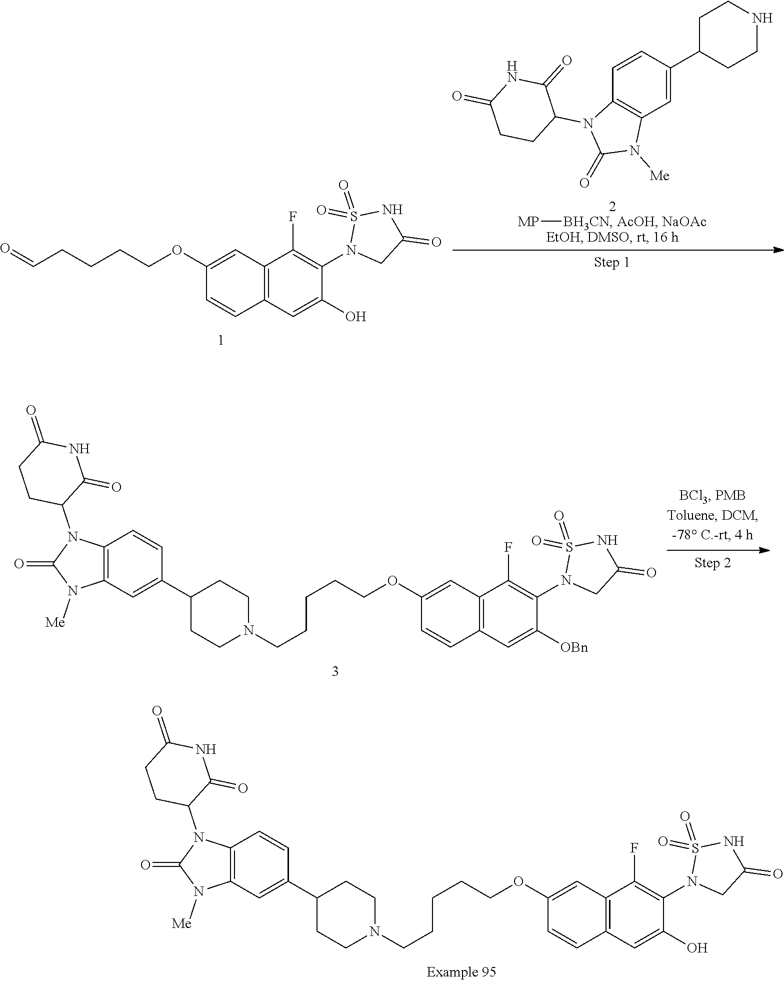

### Step 1: 3-[5-[1-[5-[[6-benzyloxy-8-fluoro-7-(1,1,4-trioxo-1,2,5-thiadiazolidin-2-yl)-2-naphthyl]oxy]pentyl]-4-piperidyl]-3-methyl-2-oxo-benzimidazol-1-yl]piperidine-2,6-dione (3)

Into a 50 mL single neck round bottom flask containing a well-stirred solution of 5-[[6-benzyloxy-8-fluoro-7-(1,1,4-trioxo-1,2,5-thiadiazolidin-2-yl)-2-naphthyl]oxy]pentanal (1, 250 mg, 157.76 μmol, crude) and 3-[3-methyl-2-oxo-5-(4-piperidyl)benzimidazol-1-yl]piperidine-2,6-dione (2, 89.65 mg, 236.63 μmol, HCl salt) in anhydrous DMSO (3 mL) and ethanol (7 mL) were added acetic acid (1.05 g, 17.48 mmol, 1 mL), sodium acetate (38.82 mg, 473.27 μmol) and MP-cyano borohydride (300 mg, 600 μmol). The suspension was stirred at RT for 16 h. The reaction mixture was filtered through a sintered funnel and concentrated under reduced pressure. The material was purified by reverse phase column chromatography [Purification method: Biotage C18 column; Mobile phase A: 0.1% TFA in water; Mobile phase B: MeCN) to afford 3-[5-[1-[5-[[6-benzyloxy-8-fluoro-7-(1,1,4-trioxo-1,2,5-thiadiazolidin-2-yl)-2-naphthyl]oxy]pentyl]-4-piperidyl]-3-methyl-2-oxo-benzimidazol-1-yl]piperidine-2,6-dione (3, 120 mg, 92.69 μmol, 59% yield, TFA salt) as off white solid. LCMS (ES+): m/z 813.2 [M+H]⁺

### Step 2: 3-[5-[1-[5-[[8-fluoro-6-hydroxy-7-(1,1,4-trioxo-1,2,5-thiadiazolidin-2-yl)-2-naphthyl]oxy]pentyl]-4-piperidyl]-3-methyl-2-oxo-benzimidazol-1-yl]piperidine-2,6-dione (Example 95)

Into a 100 mL single neck round bottom flask containing well-stirred solution of 3-[5-[1-[5-[[6-benzyloxy-8-fluoro-7-(1,1,4-trioxo-1,2,5-thiadiazolidin-2-yl)-2-naphthyl]oxy]pentyl]-4-piperidyl]-3-methyl-2-oxo-benzimidazol-1-yl]piperidine-2,6-dione (3, 120 mg, 92.69 μmol, TFA salt) and pentamethylbenzene (68.71 mg, 463.47 μmol, 74.93 μL) in anhydrous toluene (4 mL) and DCM (6 mL) was added boron trichloride (1.0 M in DCM) (217.22 mg, 1.85 mmol, 1.85 mL) at −78° C. The reaction mixture was stirred at RT for 4 h. The reaction mixture was quenched with 2 mL of 5% MeOH in DCM at −78° C. and the solvents removed under reduced pressure. The residue was washed with diethyl ether (30 mL) and purified by reverse phase prep HPLC [Purification method: Column: X-Select C18 (150×19) 5 micron; Mobile phase A: 0.1% TFA in water; Mobile phase B: MeCN] to afford 3-[5-[1-[5-[[8-fluoro-6-hydroxy-7-(1,1,4-trioxo-1,2,5-thiadiazolidin-2-yl)-2-naphthyl]oxy]pentyl]-4-piperidyl]-3-methyl-2-oxo-benzimidazol-1-yl]piperidine-2,6-dione (Example 95, 35 mg, 40.68 μmol, 44% yield, TFA salt) as an off-white solid. LCMS (ES+): m/z 723.3 [M+H]⁺

¹H NMR (400 MHZ, DMSO-d6): δ 11.11 (s, 1H), 9.81 (s, 1H), 9.07 (s, 1H), 7.70 (d, J=9.20 Hz, 1H), 7.24 (s, 1H), 7.21-7.17 (m, 1H), 7.17-7.05 (m, 2H), 6.99-6.91 (m, 1H), 5.37-5.36 (m, 1H), 4.23 (s, 2H), 4.12 (t, J=6.40 Hz, 2H), 3.63-3.53 (m, 2H), 3.35 (s, 3H), 3.14-3.04 (m, 4H), 2.91-2.87 (m, 2H), 2.72-2.61 (m, 2H), 2.06-2.01 (m, 3H), 1.91-1.76 (m, 6H), 1.54-0.95 (m, 2H).

### 2-[4-[4-[(2,6-dioxo-3-piperidyl)amino]-3-phenoxy-phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 96)

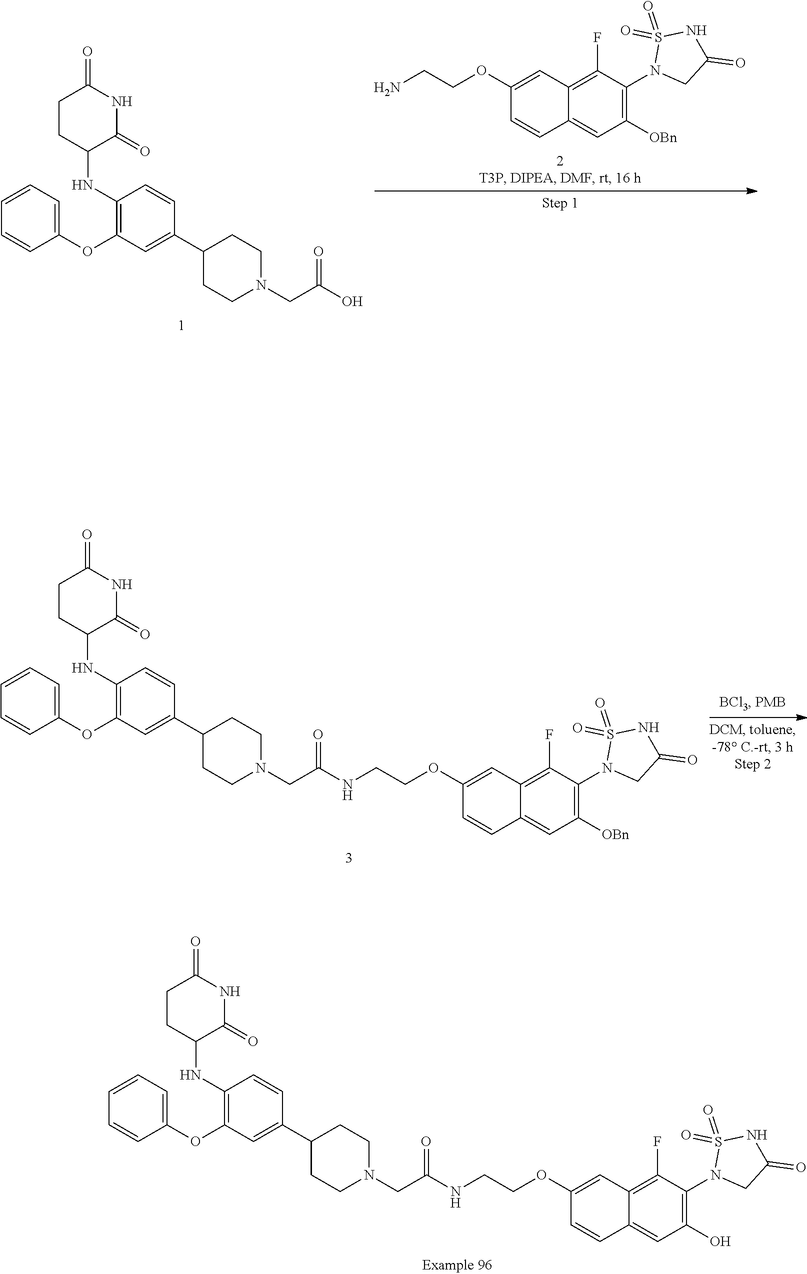 [SMILES: O=C(O)CN1CCC(c2ccc(NC3CCC(=O)NC3=O)c(Oc3ccccc3)c2)CC1 ; NCCOc1ccc2cc(OCc3ccccc3)c(N3CC(=O)NS3(=O)=O)c(F)c2c1 ; O=C(CN1CCC(c2ccc(NC3CCC(=O)NC3=O)c(Oc3ccccc3)c2)CC1)NCCOc1ccc2cc(OCc3ccccc3)c(N3CC(=O)NS3(=O)=O)c(F)c2c1 ; O=C(CN1CCC(c2ccc(NC3CCC(=O)NC3=O)c(Oc3ccccc3)c2)CC1)NCCOc1ccc2cc(O)c(N3CC(=O)NS3(=O)=O)c(F)c2c1]

### Step 1: N-[2-[[6-benzyloxy-8-fluoro-7-(1,1,4-trioxo-1,2,5-thiadiazolidin-2-yl)-2-naphthyl]oxy]ethyl]-2-[4-[4-[(2,6-dioxo-3-piperidyl)amino]-3-phenoxy-phenyl]-1-piperidyl]acetamide (3)

Into a 20 mL vial containing a well-stirred solution of 2-[4-[4-[(2,6-dioxo-3-piperidyl)amino]-3-phenoxy-phenyl]-1-piperidyl]acetic acid (1, 140 mg, 249.03 μmol, TFA salt) in DMF (2 mL) was added 5-[7-(2-aminoethoxy)-3-benzyloxy-1-fluoro-2-naphthyl]-1,1-dioxo-1,2,5-thiadiazolidin-3-one (2, 139.33 mg, 249.03 μmol, TFA salt) and DIPEA (96.55 mg, 747.08 μmol, 130.13 μL) followed by 1-propanephosphonic anhydride (50% in ethyl acetate) (118.85 mg, 373.54 μmol, 0.242 mL). After 16 h, the solvent was removed under reduced pressure and the residue was subjected to reverse phase column chromatography [Purification method: Biotage 120 g, C18 column; Mobile phase A: 0.1% TFA in water; Mobile phase B: acetonitrile) to afford N-[2-[6-benzyloxy-8-fluoro-7-(1,1,4-trioxo-1,2,5-thiadiazolidin-2-yl)-2-naphthyl]oxy]ethyl]-2-[4-[4-[(2,6-dioxo-3-piperidyl)amino]-3-phenoxy-phenyl]-1-piperidyl]acetamide (3, 100 mg, 97.77 μmol, 39% yield, TFA salt) as an off-white solid. LCMS (ES+): m/z 865.3 [M+H]⁺

### Step 2: 2-[4-[4-[(2,6-dioxo-3-piperidyl)amino]-3-phenoxy-phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 96)

Into a 25 mL single neck round bottom flask containing a well-stirred solution of N-[2-[[6-benzyloxy-8-fluoro-7-(1,1,4-trioxo-1,2,5-thiadiazolidin-2-yl)-2-naphthyl]oxy]ethyl]-2-[4-[4-[(2,6-dioxo-3-piperidyl)amino]-3-phenoxy-phenyl]-1-piperidyl]acetamide (3, 90 mg, 87.34 μmol, TFA salt) in DCM (2.5 mL) and toluene (2.5 mL) was added pentamethylbenzene (64.74 mg, 436.69 μmol, 70.60 μL) and the reaction mixture was cooled to −78° C. Then, boron trichloride (1.0 M solution in DCM) (204.67 mg, 1.75 mmol, 2 mL) was added dropwise over a period of 2 min, subsequently the reaction mixture was brought to RT and stirred for 3 h. The reaction mixture was cooled to −78° C. and quenched slowly with 10% methanol in DCM (1.5 mL). The reaction mixture was concentrated under reduced pressure at 30° C. The residue was triturated with diethyl ether (10 mL), filtered and purified by reverse phase prep HPLC [Purification method: Column: X-bridge, C18 (150×19) mm, 5 micron; Mobile phase A: 0.1% TFA in water and Mobile phase B: MeCN] to afford 2-[4-[4-[(2,6-dioxo-3-piperidyl)amino]-3-phenoxy-phenyl]-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example, 28.68 mg, 30.01 μmol, 34% yield, TFA salt) as an off-white solid. LCMS (ES+): m/z 775.2 [M+H]⁺

¹H NMR (400 MHZ, DMSO-d6): δ 10.85 (s, 1H), 9.69 (s, 1H), 9.57 (s, 1H), 8.85 (t, J=5.20 Hz, 1H), 7.70 (d, J=8.80 Hz, 1H), 7.39-7.35 (m, 2H), 7.23 (d, J=2.40 Hz, 1H), 7.08-7.15 (m, 5H), 6.99-6.93 (m, 2H), 6.68 (d, J=1.60 Hz, 1H), 5.34 (s, 1H), 4.40-4.35 (m, 1H), 4.18-4.12 (m, 4H), 3.93 (s, 2H), 3.60-3.40 (m, 3H), 3.06-3.00 (m, 2H), 2.64-2.82 (m, 3H), 2.13-2.09 (m, 1H), 2.07-1.80 (m, 5H).

### 2-[4-[1-(2,6-dioxo-3-piperidyl)-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]-N-methyl-acetamide (Example 97)

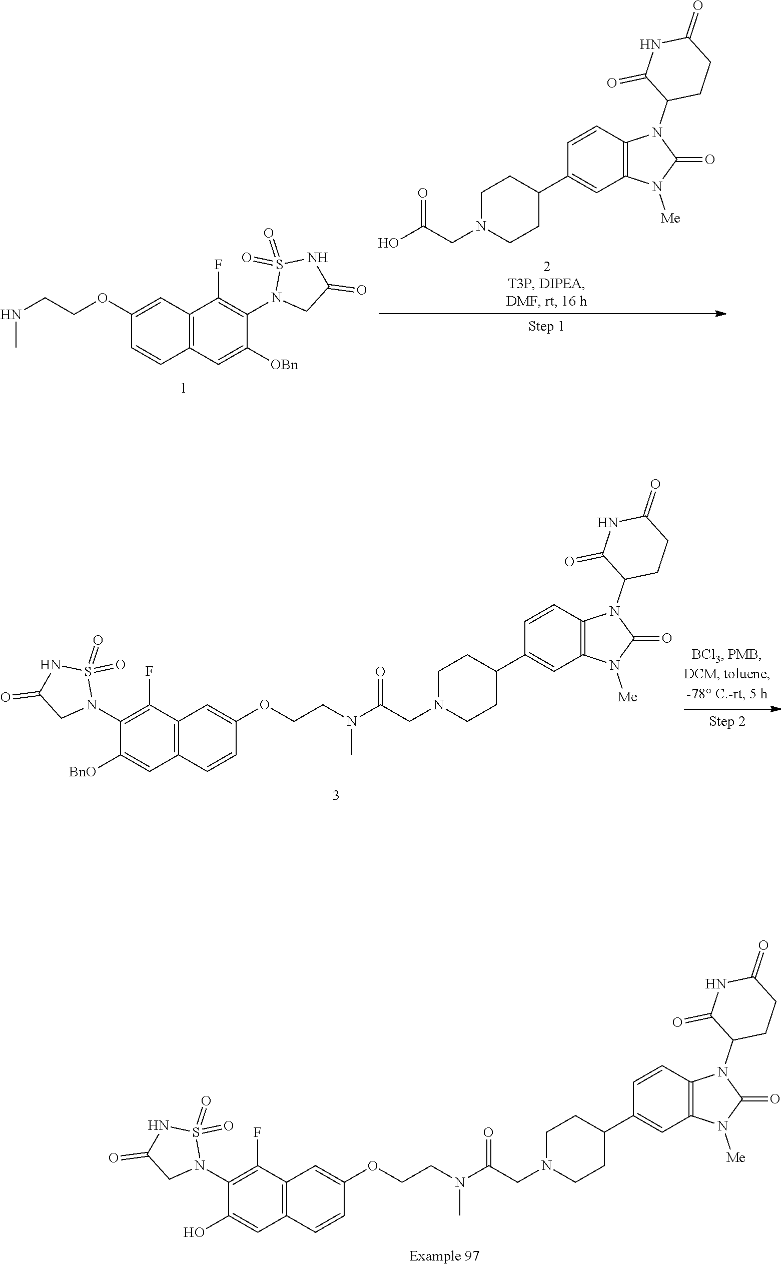 [SMILES: CNCCOc1ccc2cc(OCc3ccccc3)c(N3CC(=O)NS3(=O)=O)c(F)c2c1 ; Cn1c(=O)n(C2CCC(=O)NC2=O)c2ccc(C3CCN(CC(=O)O)CC3)cc21 ; CN(CCOc1ccc2cc(OCc3ccccc3)c(N3CC(=O)NS3(=O)=O)c(F)c2c1)C(=O)CN1CCC(c2ccc3c(c2)n(C)c(=O)n3C2CCC(=O)NC2=O)CC1 ; CN(CCOc1ccc2cc(O)c(N3CC(=O)NS3(=O)=O)c(F)c2c1)C(=O)CN1CCC(c2ccc3c(c2)n(C)c(=O)n3C2CCC(=O)NC2=O)CC1]

### Step 1: N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-3-methyl-2-oxo-benzimidazol-5-yl]-1-piperidyl]-N-methyl-acetamide (3)

Into a 50 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (2, 125.68 mg, 244.29 μmol, TFA salt) and 5-[3-benzyloxy-1-fluoro-7-[2-(methylamino)ethoxy]-2-naphthyl]-1,1-dioxo-1,2,5-thiadiazolidin-3-one (1, 180) mg, 313.86 μmol) in DMF (4 mL) was added N,N-diisopropylethylamine (222.60 mg, 1.72 mmol, 0.3 mL) and propylphosphonic anhydride solution (50% in EtOAc) (299.61 mg, 941.57 μmol, 0.3 mL). After 16h. the volatiles were removed under reduced pressure. The residue was purified by reverse phase prep HPLC [Purification method: Column: Siliasep premium C18, 25 μm, 120 g, Mobile phase A: 0.1% TFA in water; Mobile phase B: Acetonitrile) to afford N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-3-methyl-2-oxo-benzimidazol-5-yl]-1-piperidyl]-N-methyl-acetamide (3, 200 mg, 205.46 μmol, 65% yield, TFA salt) as a pale yellow solid. LCMS (ES+): m/z 842.2 [M+H]⁺

### Step 2: 2-[4-[1-(2,6-dioxo-3-piperidyl)-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]-N-methyl-acetamide (Example 97)

Into a 50 mL single neck round bottom flask containing a well-stirred solution of N-[2-[[6-benzyloxy-8-fluoro-7-(1,1,4-trioxo-1,2,5-thiadiazolidin-2-yl)-2-naphthyl]oxy]ethyl]-2-[4-[1-(2,6-dioxo-3-piperidyl)-3-methyl-2-oxo-benzimidazol-5-yl]-1-piperidyl]-N-methyl-acetamide (3, 180 mg, 213.80 μmol, TFA salt) in anhydrous DCM (3 mL) and toluene (3 mL) was added pentamethylbenzene (139.57 mg, 941.50 μmol). The solution was cooled to −75° C. and treated with boron trichloride (1.0 M in DCM) (3.76 mmol, 3.7 mL). The reaction mixture was stirred for 5 h at RT. The reaction mixture was cooled to −75° C. and quenched with 5% MeOH in DCM (1.5 mL). The volatiles were removed under reduced pressure and the residue was triturated with diethyl ether (10 mL) and purified by reverse phase prep HPLC [Purification method: Column: X-SELECT C18 (19×150 mm), 5 mic; Mobile phase A: 0.1% TFA in water; Mobile phase B: MeCN] to obtain 2-[4-[1-(2,6-dioxo-3-piperidyl)-3-methyl-2-oxo-benzimidazol-5-yl]-1-piperidyl]-N-[2-[[8-fluoro-6-hydroxy-7-(1,1,4-trioxo-1,2,5-thiadiazolidin-2-yl)-2-naphthyl]oxy]ethyl]-N-methyl-acetamide (Example 97, 100 mg, 113.34 μmol, 53% yield, TFA salt) as an off-white solid. LCMS (ES+): m/z 752.2 [M+H]⁺

¹H NMR (400 MHZ, DMSO-d6): δ 11.11 (s, 1H), 9.86 (s, 1H), 9.50 (s, 1H), 7.75-7.71 (m, 1H), 7.28-7.23 (m, 1H), 7.18-7.08 (m, 4H), 7.00-6.90 (m, 1H), 5.39-5.34 (m, 1H), 4.44-4.43 (m, 1H), 4.34-4.24 (m, 5H), 3.84-3.76 (m, 2H), 3.36 (s, 3H), 3.21-3.04 (m, 5H), 2.92-2.87 (m, 2H), 2.73-2.65 (m, 2H), 2.20-1.95 (m, 5H).

### 3-[5-[1-[6-[[8-fluoro-6-hydroxy-7-(1,1,4-trioxo-1,2,5-thiadiazolidin-2-yl)-2-naphthyl]oxy]hexyl]-4-piperidyl]-3-methyl-2-oxo-benzimidazol-1-yl]piperidine-2,6-dione (Example 98)

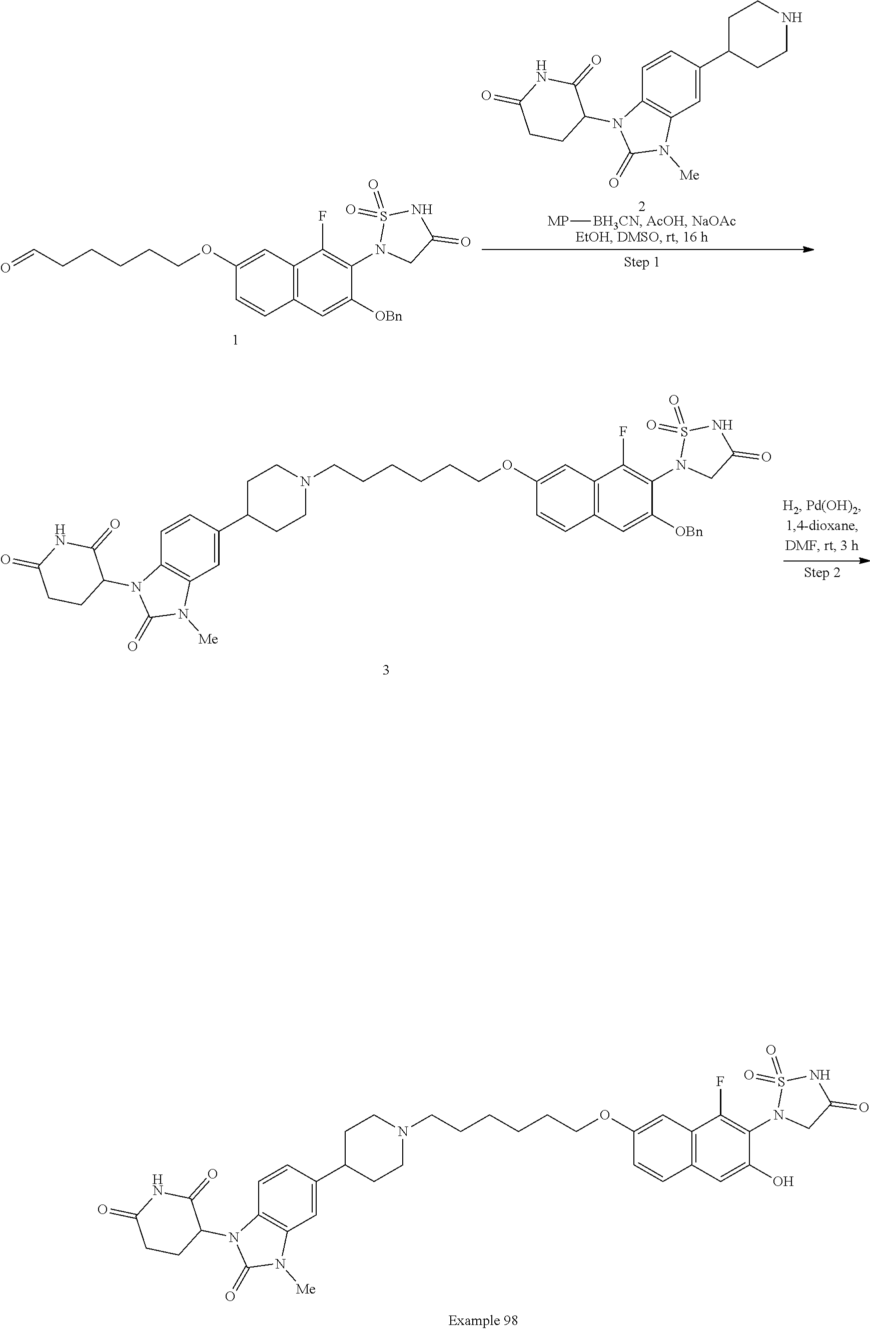

### Step 1: 3-[5-[1-[6-[[6-benzyloxy-8-fluoro-7-(1,1,4-trioxo-1,2,5-thiadiazolidin-2-yl)-2-naphthyl]oxy]hexyl]-4-piperidyl]-3-methyl-2-oxo-benzimidazol-1-yl]piperidine-2,6-dione (3)

Into a 50 mL single neck round bottom flask containing a well-stirred solution of 3-[3-methyl-2-oxo-5-(4-piperidyl)benzimidazol-1-yl]piperidine-2,6-dione (2, 120 mg, 316.75 μmol, HCl salt) in ethanol (3 mL) and anhydrous DMSO (1 mL) were added anhydrous sodium acetate (77.95 mg, 950.24 μmol, 50.95 μL), 6-[[6-benzyloxy-8-fluoro-7-(1,1,4-trioxo-1,2,5-thiadiazolidin-2-yl)-2-naphthyl]oxy]hexanal (1, 290.66 mg, 348.42 μmol), AcOH (190.21 mg, 3.17 mmol, 181.15 μL) and MP-cyanoborohydride (15 mg, 316.75 μmol). After 16 h, the reaction mixture was filtered and the solvent removed under reduced pressure. The residue was subjected to reverse phase column chromatography [Purification method: Siliasep premium C18, 25 μm, 120 g; Mobile phase: 0.1% TFA in water; Mobile phase B: Acetonitrile] to afford 3-[5-[1-[6-[[6-benzyloxy-8-fluoro-7-(1,1,4-trioxo-1,2,5-thiadiazolidin-2-yl)-2-naphthyl]oxy]hexyl]-4-piperidyl]-3-methyl-2-oxo-benzimidazol-1-yl]piperidine-2,6-dione TFA salt (120 mg, 126.25 μmol, 40% yield) as an off-white solid. LCMS (ES+): m/z 827.2 [M+H]⁺

### Step 2: 3-[5-[1-[6-[[8-fluoro-6-hydroxy-7-(1,1,4-trioxo-1,2,5-thiadiazolidin-2-yl)-2-naphthyl]oxy]hexyl]-4-piperidyl]-3-methyl-2-oxo-benzimidazol-1-yl]piperidine-2,6-dione (Example 98)

Into a 25 mL single neck round bottom flask containing a well-stirred solution of 3-[5-[1-[6-[[6-benzyloxy-8-fluoro-7-(1,1,4-trioxo-1,2,5-thiadiazolidin-2-yl)-2-naphthyl]oxy]hexyl]-4-piperidyl]-3-methyl-2-oxo-benzimidazol-1-yl]piperidine-2,6-dione (3, 120 mg, 143.66 μmol) in anhydrous DMF (0.5 mL) and 1,4-dioxane (1 mL) was added palladium hydroxide (20 wt. %, 50% water on carbon) (151.32 mg, 215.50 μmol, 20% purity). The reaction mixture was stirred at RT under hydrogen atmosphere (bladder) for 16 h. The reaction mixture was filtered washed with 15% TFA in 1,4-dioxane (100 mL). The solvent was removed and the residue was purified by reverse phase column chromatography [Purification method: Biotage Ultra C18 (30 g, 25 μm): Mobile phase A: 0.1% TFA in water; Mobile phase B: Acetonitrile] to afford 3-[5-[1-[6-[[8-fluoro-6-hydroxy-7-(1,1,4-trioxo-1,2,5-thiadiazolidin-2-yl)-2-naphthyl]oxy]hexyl]-4-piperidyl]-3-methyl-2-oxo-benzimidazol-1-yl]piperidine-2,6-dione TFA salt (Example 98, 32 mg, 35.86 μmol, 25% yield) as an off-white solid. LCMS (ES+): m/z 737.2 [M+H]⁺

¹H NMR (400 MHZ, DMSO-d6): δ 11.07 (s, 1H), 9.68 (s, 1H), 9.01 (s, 1H), 7.68 (d, J=9.20 Hz, 1H), 7.30 (s, 1H), 7.27-7.24 (m, 1H), 7.19-6.98 (m, 3H), 6.92 (d, J=8.40 Hz, 1H), 5.36-5.32 (m, 1H), 4.16 (s, 2H), 4.10 (t, J=6.00 Hz, 2H), 3.70-3.60 (m, 5H), 3.13-3.03 (m, 6H), 2.90-2.87 (m, 3H), 2.10-2.02 (m, 3H), 1.83-1.72 (m, 6H), 1.52-1.41 (m, 4H).

### N-[[1-[1-(2,6-dioxo-3-piperidyl)-3-methyl-2-oxo-benzimidazol-5-yl]-4-piperidyl]methyl]-5-fluoro-7-hydroxy-6-(1,1,4-trioxo-1,2,5-thiadiazolidin-2-yl)naphthalene-2-carboxamide (Example 99)

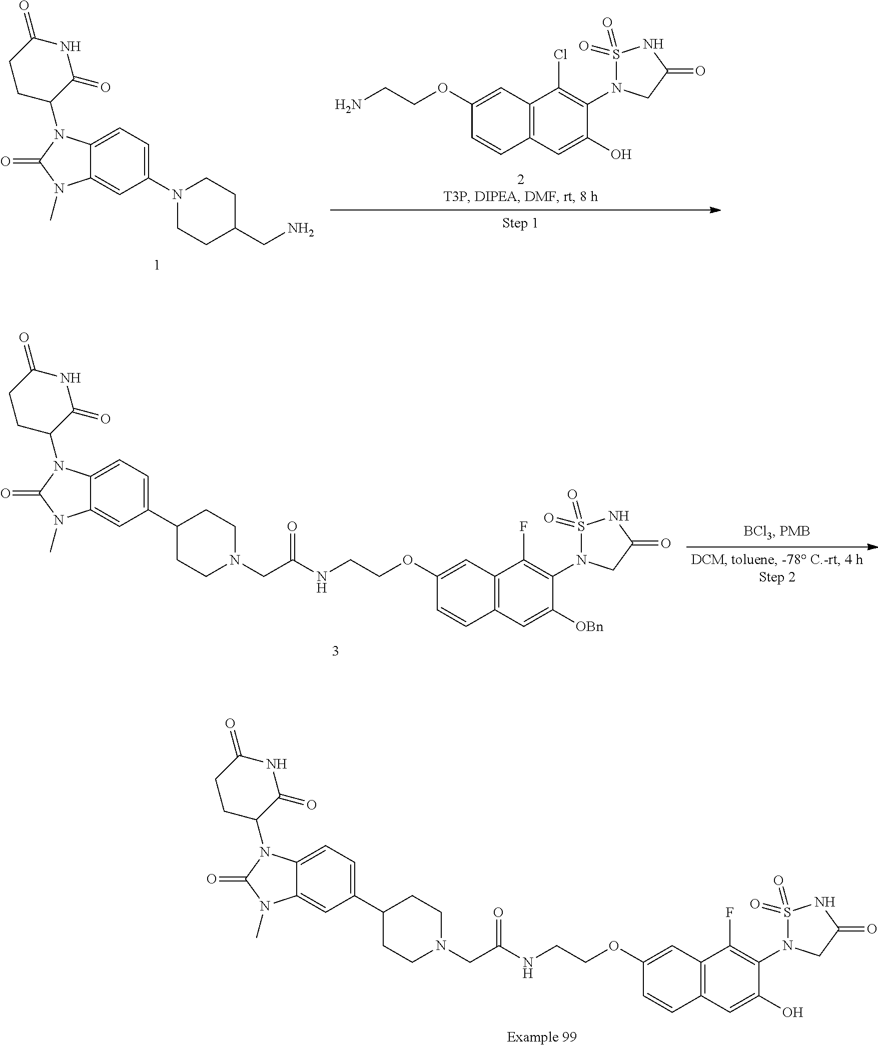 [SMILES: Cn1c(=O)n(C2CCC(=O)NC2=O)c2ccc(N3CCC(CN)CC3)cc21 ; NCCOc1ccc2cc(O)c(N3CC(=O)NS3(=O)=O)c(Cl)c2c1 ; Cn1c(=O)n(C2CCC(=O)NC2=O)c2ccc(C3CCN(CC(=O)NCCOc4ccc5cc(OCc6ccccc6)c(N6CC(=O)NS6(=O)=O)c(F)c5c4)CC3)cc21 ; Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3)cc21]

### Step 1: 7-benzyloxy-N-[[1-[1-(2,6-dioxo-3-piperidyl)-3-methyl-2-oxo-benzimidazol-5-yl]-4-piperidyl]methyl]-5-fluoro-6-(1,1,4-trioxo-1,2,5-thiadiazolidin-2-yl)naphthalene-2-carboxamide (3)

Into a 50 mL single neck round bottom flask containing well-stirred solution of 7-benzyloxy-5-fluoro-6-(1,1,4-trioxo-1,2,5-thiadiazolidin-2-yl)naphthalene-2-carboxylic acid (2, 150 mg, 340.63 μmol) in anhydrous DMF (4 mL) were added propylphosphonic anhydride solution (≥50 wt. % in ethyl acetate) (498.56 mg, 783.45 μmol, 0.5 mL) and DIPEA (220.12 mg, 1.70 mmol, 296.66 μL). After 15 min, 3-[5-[4-(aminomethyl)-1-piperidyl]-3-methyl-2-oxo-benzimidazol-1-yl]piperidine-2,6-dione (1, 171.93 mg, 340.63 μmol, TFA salt). After 8 h, the solvent was removed under reduced pressure. The residue was purified by reverse phase column chromatography [Purification method: Biotage C-18 column; Mobile phase A: 0.1% TFA in water; Mobile phase B: MeCN] to afford 7-benzyloxy-N-[[1-[1-(2,6-dioxo-3-piperidyl)-3-methyl-2-oxo-benzimidazol-5-yl]-4-piperidyl]methyl]-5-fluoro-6-(1,1,4-trioxo-1,2,5-thiadiazolidin-2-yl)naphthalene-2-carboxamide (3, 180 mg, 158.28 μmol, 46% yield, TFA salt) as off white solid. LCMS (ES+): m/z 784.4 [M+H]⁺

### Step 2: N-[[1-[1-(2,6-dioxo-3-piperidyl)-3-methyl-2-oxo-benzimidazol-5-yl]-4-piperidyl]methyl]-5-fluoro-7-hydroxy-6-(1,1,4-trioxo-1,2,5-thiadiazolidin-2-yl)naphthalene-2-carboxamide (Example 99)

Into a 50 mL single neck round bottom flask containing a well-stirred solution of 7-benzyloxy-N-[[1-[1-(2,6-dioxo-3-piperidyl)-3-methyl-2-oxo-benzimidazol-5-yl]-4-piperidyl]methyl]-5-fluoro-6-(1,1,4-trioxo-1,2,5-thiadiazolidin-2-yl)naphthalene-2-carboxamide (3, 180 mg, 181.30 μmol) and pentamethylbenzene (134.39 mg, 906.52 μmol, 146.55 μL) in anhydrous DCM (7 mL) and toluene (7 mL) was added boron trichloride (1.0 M in DCM) (424.87 mg, 3.63 mmol) at −78° C. Then the reaction mixture was stirred at RT for 4 h. The reaction was quenched with 5% MeOH in DCM (2 mL) at −78° C. The volatiles were removed under reduced pressure. The residue was triturated with diethyl ether (30 mL) and filtered. The material was purified by reverse phase prep HPLC [Purification method: Column: X-Select C18 (150×19) 5 micron; Mobile phase A: 0.1% formic acid in water. Mobile phase B: MeCN] to afford N-[[1-[1-(2,6-dioxo-3-piperidyl)-3-methyl-2-oxo-benzimidazol-5-yl]-4-piperidyl]methyl]-5-fluoro-7-hydroxy-6-(1,1,4-trioxo-1,2,5-thiadiazolidin-2-yl)naphthalene-2-carboxamide (Example 99, 80 mg, 114.04 μmol, 63% yield) as a colorless solid. LCMS (ES+): m/z 694.2 [M+H]⁺

¹H NMR (400 MHZ, DMSO-d₆): δ 11.15 (s, 1H), 10.40 (s, 1H), 8.84 (t, J=5.60 Hz, 1H), 8.27 (s, 1H), 7.97 (d, J=8.40 Hz, 1H), 7.80 (dd, J=1.20, 8.80 Hz, 1H), 7.48 (s, 1H), 7.27-7.20 (m, 3H), 5.42-5.37 (m, 1H), 4.23 (s, 2H), 3.66-3.52 (m, 4H), 3.37-3.34 (m, 6H), 2.93-2.86 (m, 1H), 2.73-2.62 (m, 2H), 2.04-2.00 (m, 4H), 1.67-1.64 (m, 2H).

### N-[2-[[8-chloro-6-hydroxy-7-(1,1,4-trioxo-1,2,5-thiadiazolidin-2-yl)-2-naphthyl]oxy]ethyl]-2-[4-[1-(2,6-dioxo-3-piperidyl)-3-methyl-2-oxo-benzimidazol-5-yl]-1-piperidyl]acetamide (Example 100)

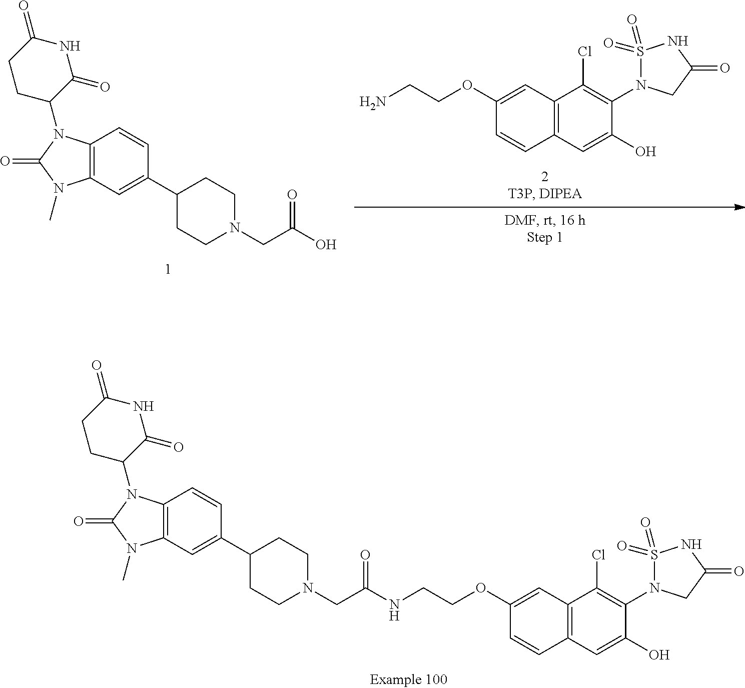 [SMILES: Cn1c(=O)n(C2CCC(=O)NC2=O)c2ccc(C3CCN(CC(=O)O)CC3)cc21 ; NCCOc1ccc2cc(O)c(N3CC(=O)NS3(=O)=O)c(Cl)c2c1 ; Cn1c(=O)n(C2CCC(=O)NC2=O)c2ccc(C3CCN(CC(=O)NCCOc4ccc5cc(O)c(N6CC(=O)NS6(=O)=O)c(Cl)c5c4)CC3)cc21]

### Step 1: N-[2-[[8-chloro-6-hydroxy-7-(1,1,4-trioxo-1,2,5-thiadiazolidin-2-yl)-2-naphthyl]oxy]ethyl]-2-[4-[1-(2,6-dioxo-3-piperidyl)-3-methyl-2-oxo-benzimidazol-5-yl]-1-piperidyl]acetamide (Example 100)

Into a 10 mL single neck round bottom flask containing well-stirred solution of 2-[4-[1-(2,6-dioxo-3-piperidyl)-3-methyl-2-oxo-benzimidazol-5-yl]-1-piperidyl]acetic acid (1, 100 mg, 192.44 μmol, TFA salt) in anhydrous DMF (3 mL) were added propylphosphonic anhydride solution (≥50 wt. % in ethyl acetate) (367.38 mg, 577.32 μmol, 0.37 mL) and DIPEA (296.80 mg, 2.30 mmol, 0.4 mL). After 15 min, 5-[7-(2-aminoethoxy)-1-chloro-3-hydroxy-2-naphthyl]-1,1-dioxo-1,2,5-thiadiazolidin-3-one (2, 93.49 mg, 192.44 μmol, TFA salt) was added. After 16 h, the solvent was removed under reduced pressure. The residue was treated with water (2 mL) and the precipitate was filtered and dried. The material was purified by reverse phase prep HPLC [Purification method: Column: Zorbax C18 (250×21) 7 micron, Mobile phase A: 0.1% TFA in water, Mobile phase B: MeCN] to afford N-[2-[[8-chloro-6-hydroxy-7-(1,1,4-trioxo-1,2,5-thiadiazolidin-2-yl)-2-naphthyl]oxy]ethyl]-2-[4-[1-(2,6-dioxo-3-piperidyl)-3-methyl-2-oxo-benzimidazol-5-yl]-1-piperidyl]acetamide (Example 100, 23 mg, 25.84 μmol, 13% yield, TFA salt) as a colorless solid. LCMS (ES+): m/z 754.2 [M+H]⁺

¹H NMR (400 MHZ, DMSO-d6): δ 11.11 (s, 1H), 9.84 (s, 1H), 9.65 (s, 1H), 8.90-8.88 (m, 1H), 7.76 (d, J=9.20 Hz, 1H), 7.41 (s, 1H), 7.41-7.20 (m, 2H), 7.19-7.04 (m, 2H), 6.92 (d, J=8.00 Hz, 1H), 5.38-5.34 (m, 1H), 4.26-4.19 (m, 4H), 3.99 (s, 2H), 3.65-3.63 (m, 2H), 3.57-3.54 (m, 2H), 3.25 (s, 3H), 3.17-3.15 (m, 2H), 3.09-2.91 (m, 2H), 2.81-2.61 (m, 2H), 2.08-1.96 (m, 2H).

### 2-[4-[4-[(2,6-dioxo-3-piperidyl)amino]-3-phenoxy-phenyl]-1-piperidyl]-N-[5-fluoro-7-hydroxy-6-(1,1,4-trioxo-1,2,5-thiadiazolidin-2-yl)-2-naphthyl]acetamide (Example 101)

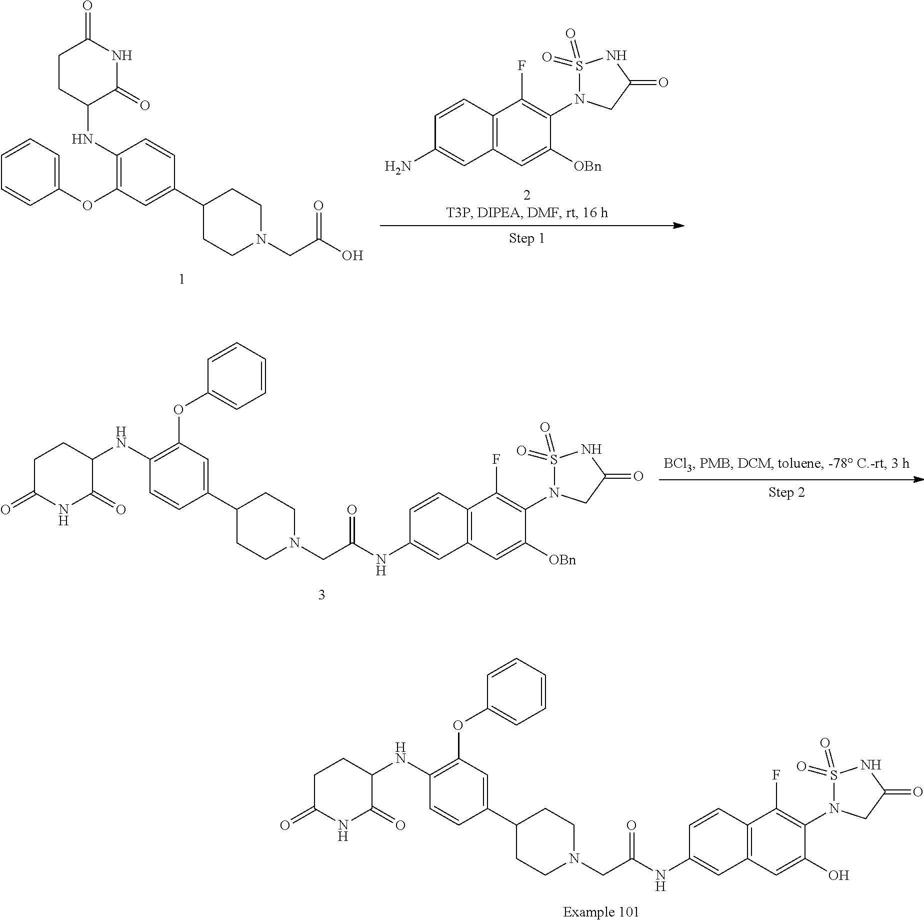 [SMILES: O=C(O)CN1CCC(c2ccc(NC3CCC(=O)NC3=O)c(Oc3ccccc3)c2)CC1 ; Nc1ccc2c(F)c(N3CC(=O)NS3(=O)=O)c(OCc3ccccc3)cc2c1 ; O=C1CCC(Nc2ccc(C3CCN(CC(=O)Nc4ccc5c(F)c(N6CC(=O)NS6(=O)=O)c(OCc6ccccc6)cc5c4)CC3)cc2Oc2ccccc2)C(=O)N1 ; O=C1CCC(Nc2ccc(C3CCN(CC(=O)Nc4ccc5c(F)c(N6CC(=O)NS6(=O)=O)c(O)cc5c4)CC3)cc2Oc2ccccc2)C(=O)N1]

### Step 1: N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)amino]-3-phenoxy-phenyl]-1-piperidyl]acetamide (3)

Into a 20 mL capped vial containing a well-stirred solution of 2-[4-[4-[(2,6-dioxo-3-piperidyl)amino]-3-phenoxy-phenyl]-1-piperidyl]acetic acid (1, 140 mg, 243.69 μmol, TFA salt) in anhydrous DMF (2 mL) were added 5-(6-amino-3-benzyloxy-1-fluoro-2-naphthyl)-1,1-dioxo-1,2,5-thiadiazolidin-3-one (2, 135.73 mg, 243.69 μmol, TFA salt) and N,N-diisopropylethylamine (94.49 mg, 731.08 μmol, 127.34 μL) followed by 1-propanephosphonic anhydride solution (50% in ethyl acetate) (116.31 mg, 365.54 μmol, 0.242 mL). After 16 h, the solvent was removed under reduced pressure and the residue was subjected to reverse phase column chromatography [Purification method: Biotage C18 column; Mobile phase A: 0.1% TFA in water; Mobile phase B: Acetonitrile] to afford N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)amino]-3-phenoxy-phenyl]-1-piperidyl]acetamide (3, 60 mg, 62.69 μmol, 26% yield, TFA salt) as an off white solid. LCMS (ES+): m/z 821.5 [M+H]⁺

### Step 2: 2-[4-[4-[(2,6-dioxo-3-piperidyl)amino]-3-phenoxy-phenyl]-1-piperidyl]-N-[5-fluoro-7-hydroxy-6-(1,1,4-trioxo-1,2,5-thiadiazolidin-2-yl)-2-naphthyl]acetamide (Example 101)

Into a 25 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)amino]-3-phenoxy-phenyl]-1-piperidyl]acetamide (3, 60 mg, 62.25 μmol, TFA salt) in anhydrous DCM (2 mL) and toluene (2 mL) was added pentamethylbenzene (46.14 mg, 311.26 μmol, 50.32 μL) and the reaction mixture was cooled to −78° C. Then, boron trichloride (1.0M in DCM) (145.88 mg, 1.25 mmol, 1.5 mL) was added dropwise over a period of 2 min. The reaction mixture was stirred at RT. After 3 h, the reaction mixture was cooled to −78° C. and quenched slowly with 10% methanol in DCM (1 mL). The reaction mixture was concentrated under reduced pressure, triturated with diethyl ether (10 mL), filtered, dried and purified by reverse phase prep HPLC [Purification method: Column: X-bridge, C18 (150×19) mm, 5 micron; Mobile phase A: 0.1% TFA in water; Mobile phase B: MeCN] to afford 2-[4-[4-[(2,6-dioxo-3-piperidyl)amino]-3-phenoxy-phenyl]-1-piperidyl]-N-[5-fluoro-7-hydroxy-6-(1,1,4-trioxo-1,2,5-thiadiazolidin-2-yl)-2-naphthyl]acetamide (Example 101, 17.86 mg, 20.84 μmol, 33% yield, TFA salt) as an off-white solid. LCMS (ES+): m/z 731.4 [M+H]⁺

¹H NMR (400 MHZ, DMSO-d6): δ 10.85 (s, 1H), 10.78 (s, 1H), 10.11 (s, 1H), 9.71 (s, 1H), 8.10 (s, 1H), 7.91 (d, J=8.80 Hz, 1H), 7.47 (d, J=2.00 Hz, 1H), 7.45-7.36 (m, 2H), 7.11 (t, J=7.20 Hz, 1H), 7.01-6.99 (m, 3H), 6.92-6.83 (m, 2H), 6.71 (d, J=1.60 Hz, 1H), 5.35 (s, 1H), 4.41-4.36 (m, 1H), 4.20 (s, 4H), 3.62-3.59 (m, 4H), 3.19-3.17 (m, 2H), 2.82-2.67 (m, 2H), 2.13-2.08 (m, 2H), 2.00-1.85 (m, 5H).

### N-[[4-[[1-(2,6-dioxo-3-piperidyl)-3-methyl-2-oxo-benzimidazol-5-yl]cyclohex-3-en-1-yl]methyl]-5-fluoro-7-hydroxy-6-(1,1,4-trioxo-1,2,5-thiadiazolidin-2-yl)naphthalene-2-carboxamide (Example 102)

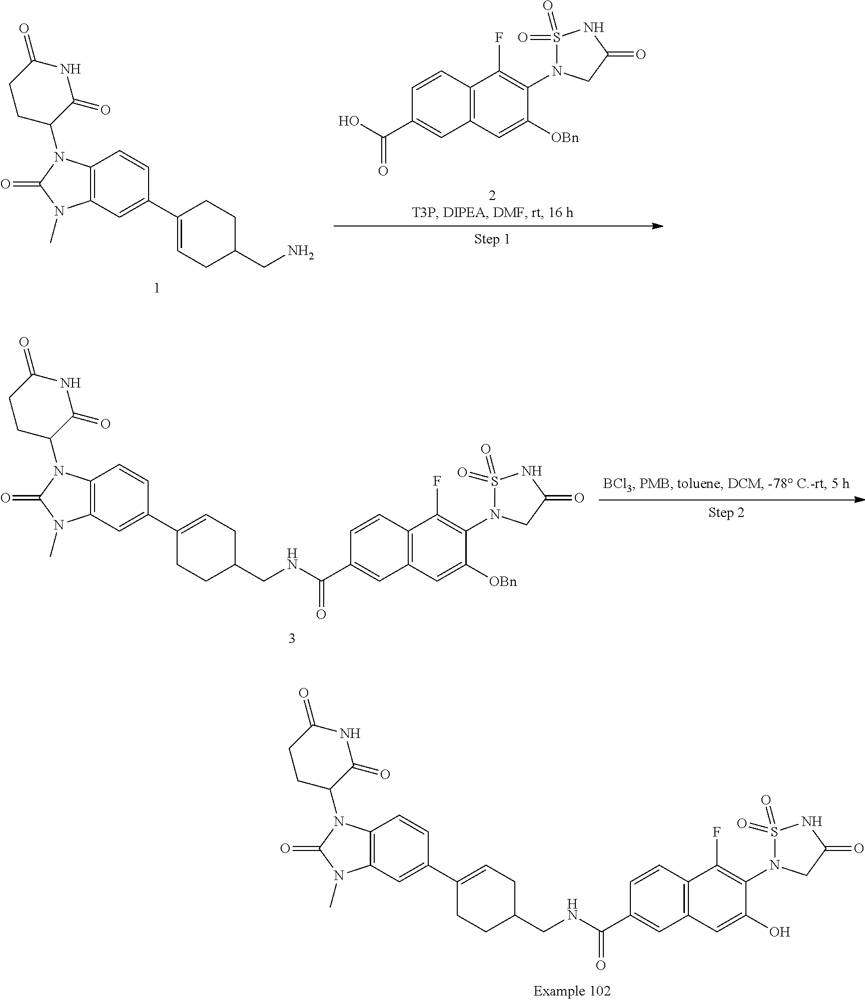 [SMILES: Cn1c(=O)n(C2CCC(=O)NC2=O)c2ccc(C3=CCC(CN)CC3)cc21 ; O=C1CN(c2c(OCc3ccccc3)cc3cc(C(=O)O)ccc3c2F)S(=O)(=O)N1 ; Cn1c(=O)n(C2CCC(=O)NC2=O)c2ccc(C3=CCC(CNC(=O)c4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3=CCC(CNC(=O)c4ccc5c(F)c(N6CC(=O)NS6(=O)=O)c(O)cc5c4)CC3)cc21]

### Step 1: 7-benzyloxy-N-[[4-[[1-(2,6-dioxo-3-piperidyl)-3-methyl-2-oxo-benzimidazol-5-yl]cyclohex-3-en-1-yl]methyl]-5-fluoro-6-(1,1,4-trioxo-1,2,5-thiadiazolidin-2-yl)naphthalene-2-carboxamide (3)

Into a 50 mL single neck round bottom flask containing a well-stirred solution of 3-[5-[4-(aminomethyl)cyclohexen-1-yl]-3-methyl-2-oxo-benzimidazol-1-yl]piperidine-2,6-dione (1, 191.86 mg, 337.33 μmol) and 7-benzyloxy-5-fluoro-6-(1,1,4-trioxo-1,2,5-thiadiazolidin-2-yl)naphthalene-2-carboxylic acid (2, 200 mg, 441.44 μmol) in DMF (7 mL) were added N,N-diisopropylethylamine (222.60 mg, 1.72 mmol, 0.3 mL) and propylphosphonic anhydride solution (50% EtOAc) (421.40 mg, 0.697 mmol, 0.4 mL). After 16 h, the reaction mixture was concentrated under reduced pressure. The residue was purified by reverse phase column chromatography [Purification method: Siliasep premium C-18, 25 μm, 120 g, Mobile phase A: 0.1% TFA in water, Mobile phase B: MeCN] to afford 7-benzyloxy-N-[[4-[[1-(2,6-dioxo-3-piperidyl)-3-methyl-2-oxo-benzimidazol-5-yl]cyclohex-3-en-1-yl]methyl]-5-fluoro-6-(1,1,4-trioxo-1,2,5-thiadiazolidin-2-yl)naphthalene-2-carboxamide (3, 180) mg, 226.84 μmol, 51% yield) as a pale yellow solid. LCMS (ES+): m/z 781.2 [M+H]⁺

### Step 2: N-[[4-[[1-(2,6-dioxo-3-piperidyl)-3-methyl-2-oxo-benzimidazol-5-yl]cyclohex-3-en-1-yl]methyl]-5-fluoro-7-hydroxy-6-(1,1,4-trioxo-1,2,5-thiadiazolidin-2-yl)naphthalene-2-carboxamide (Example 102)

Into a 50 mL single neck round bottom flask containing a well-stirred solution of 7-benzyloxy-N-[[4-[1-(2,6-dioxo-3-piperidyl)-3-methyl-2-oxo-benzimidazol-5-yl]cyclohex-3-en-1-yl]methyl]-5-fluoro-6-(1,1,4-trioxo-1,2,5-thiadiazolidin-2-yl)naphthalene-2-carboxamide (3, 180 mg, 225.92 μmol) in DCM (3 mL) and toluene (3 mL) was added pentamethylbenzene (167.46 mg, 1.13 mmol, 182.62 μL). The mixture was cooled to −75° C. and treated with boron trichloride (1.0 M, in DCM) (4 mL, 4.61 mmol). The reaction mixture was stirred at RT for 5 h. The reaction mixture was quenched with 5% MeOH in DCM (2.5 mL) at −75° C. The reaction mixture was concentrated under reduced pressure and the residue was triturated with diethyl ether (10 mL) and purified by reverse phase prep HPLC [Purification method: Column: X-Select C-18 (19×150 mm), 5 mic, Mobile phase A: 0.1% TFA in water, Mobile phase B: MeCN] to afford N-[[4-[[1-(2,6-dioxo-3-piperidyl)-3-methyl-2-oxo-benzimidazol-5-yl]cyclohex-3-en-1-yl]methyl]-5-fluoro-7-hydroxy-6-(1,1,4-trioxo-1,2,5-thiadiazolidin-2-yl)naphthalene-2-carboxamide (Example 102, 67 mg, 91.81 μmol, 41% yield) as an off white solid. LCMS (ES+): m/z 691.2 [M+H]⁺

¹H NMR (400 MHZ, DMSO-d6): δ 11.10 (s, 1H), 10.74 (s, 1H), 10.74 (m, 1H), 8.31 (s, 1H), 8.00 (d, J=11.60 Hz, 1H), 7.83 (d, J=11.60 Hz, 1H), 7.24-7.23 (m, 2H), 7.17-7.03 (m, 2H), 6.13 (s, 1H), 5.39-5.37 (m, 1H), 4.49 (s, 2H), 3.44-3.32 (m, 6H), 2.91-2.65 (m, 3H), 2.17-1.96 (m, 5H), 1.46-1.41 (m, 1H).

### 3-[5-[4-[2-[3-[8-fluoro-6-hydroxy-7-(1,1,4-trioxo-1,2,5-thiadiazolidin-2-yl)-2-naphthyl]-2,5-dihydropyrrol-1-yl]-2-oxo-ethyl]-1-piperidyl]-3-methyl-2-oxo-benzimidazol-1-yl]piperidine-2,6-dione (Example 103)

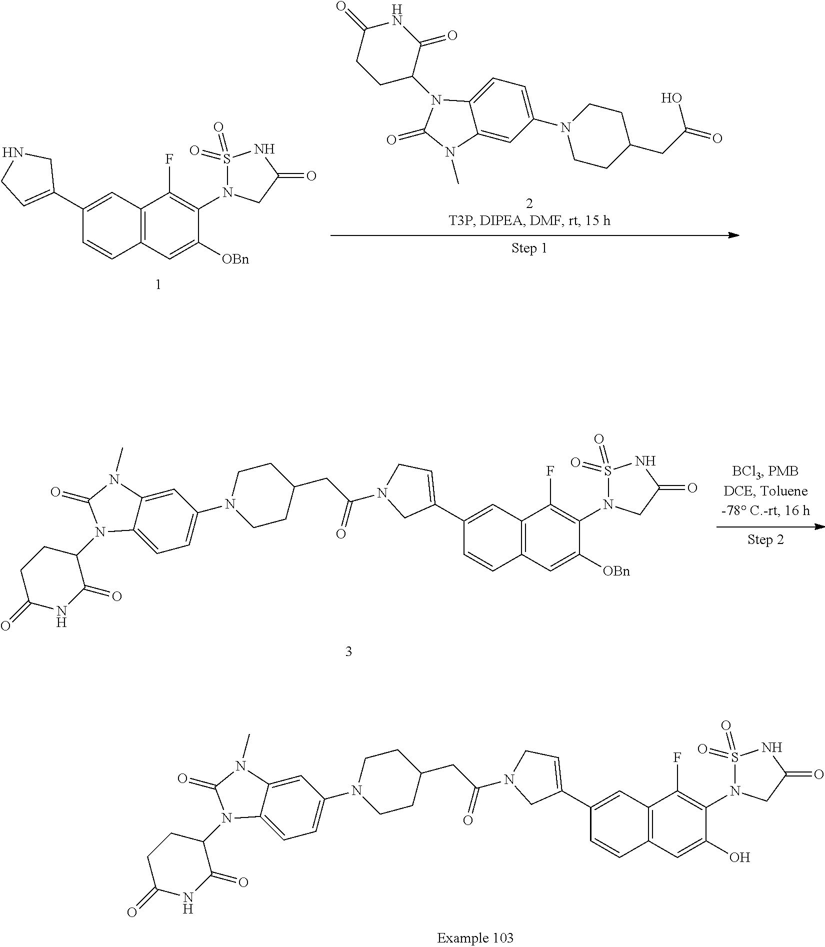 [SMILES: O=C1CN(c2c(OCc3ccccc3)cc3ccc(C4=CCNC4)cc3c2F)S(=O)(=O)N1 ; Cn1c(=O)n(C2CCC(=O)NC2=O)c2ccc(N3CCC(CC(=O)O)CC3)cc21 ; Cn1c(=O)n(C2CCC(=O)NC2=O)c2ccc(N3CCC(CC(=O)N4CC=C(c5ccc6cc(OCc7ccccc7)c(N7CC(=O)NS7(=O)=O)c(F)c6c5)C4)CC3)cc21 ; Cn1c(=O)n(C2CCC(=O)NC2=O)c2ccc(N3CCC(CC(=O)N4CC=C(c5ccc6cc(O)c(N7CC(=O)NS7(=O)=O)c(F)c6c5)C4)CC3)cc21]

### Step 1: 3-[5-[4-[2-[3-[[6-benzyloxy-8-fluoro-7-(1,1,4-trioxo-1,2,5-thiadiazolidin-2-yl)-2-naphthyl]-2,5-dihydropyrrol-1-yl]-2-oxo-ethyl]-1-piperidyl]-3-methyl-2-oxo-benzimidazol-1-yl]piperidine-2,6-dione (3)

Into a 50 mL single neck round bottom flask containing a well-stirred solution of 2-[1-[1-(2,6-dioxo-3-piperidyl)-3-methyl-2-oxo-benzimidazol-5-yl]-4-piperidyl]acetic acid (2, 200 mg, 424.55 μmol) in anhydrous DMF (5 mL) was added propanephosphonic acid anhydride (50 wt. % solution) (540 mg, 849.09 μmol) followed by N,N-diisopropylethylamine (274.34 mg, 2.12 mmol, 369.73 μL). After 1 h, 5-[3-benzyloxy-7-(2,5-dihydro-1H-pyrrol-3-yl)-1-fluoro-2-naphthyl]-1,1-dioxo-1,2,5-thiadiazolidin-3-one (1, 253.61 mg, 424.55 μmol, TFA salt) was added. After 15 h, the volatiles were removed under reduced pressure and the residue was purified using reverse phase column chromatography [Purification method: Silicycle C18 column; Mobile phase A: 0.1% TFA in water; Mobile phase B: Acetonitrile] to afford 3-[5-[4-[2-[3-[[6-benzyloxy-8-fluoro-7-(1,1,4-trioxo-1,2,5-thiadiazolidin-2-yl)-2-naphthyl]-2,5-dihydropyrrol-1-yl]-2-oxo-ethyl]-1-piperidyl]-3-methyl-2-oxo-benzimidazol-1-yl]piperidine-2,6-dione (3, 120 mg, 26% yield, TFA salt) as a pink solid. LCMS (ES+): m/z 836.2 [M+H]⁺

### Step 2: 3-[5-[4-[2-[3-[8-fluoro-6-hydroxy-7-(1,1,4-trioxo-1,2,5-thiadiazolidin-2-yl)-2-naphthyl]-2,5-dihydropyrrol-1-yl]-2-oxo-ethyl]-1-piperidyl]-3-methyl-2-oxo-benzimidazol-1-yl]piperidine-2,6-dione (Example 103)

Into a 25 mL single neck round bottom flask containing a well-stirred solution of 3-[5-[4-[2-[3-[6-benzyloxy-8-fluoro-7-(1,1,4-trioxo-1,2,5-thiadiazolidin-2-yl)-2-naphthyl]-2,5-dihydropyrrol-1-yl]-2-oxo-ethyl]-1-piperidyl]-3-methyl-2-oxo-benzimidazol-1-yl]piperidine-2,6-dione (3, 100 mg, 93.59 μmol, TFA salt) and pentamethyl benzene (69.37 mg, 467.93 μmol) in anhydrous 1,2-dichloroethane (2.5 mL) and toluene (2.5 mL) was added boron trichloride (1.0 M in DCM) (1.87 mL, 1.87 mmol) at −78° C. The reaction mixture was stirred at ambient temperature for 16 h. The reaction was quenched with 20 mL of 5% MeOH in DCM at −78° C. The volatiles were removed and the residue was purified by reverse phase prep HPLC [Purification method: Zoxbax C18 (250×21) 7 micron column; Mobile phase A: 0.1% TFA in water; Mobile phase B: Acetonitrile] to afford 3-[5-[4-[2-[3-[8-fluoro-6-hydroxy-7-(1,1,4-trioxo-1,2,5-thiadiazolidin-2-yl)-2-naphthyl]-2,5-dihydropyrrol-1-yl]-2-oxo-ethyl]-1-piperidyl]-3-methyl-2-oxo-benzimidazol-1-yl]piperidine-2,6-dione (Example 103, 45 mg, 54% yield, TFA salt) as a pale pink solid. LCMS (ES+):

m/z 746.2 [M+H]⁺

¹H NMR (400 MHZ, DMSO-d₆): δ 11.15 (s, 1H), 10.31 (s, 1H), 7.78-7.86 (m, 3H), 7.59 (s, 1H), 7.26-7.22 (m, 2H), 7.11 (s, 1H), 6.61 (s, 1H), 5.42 (dd, J=5.20, 13.20 Hz, 1H), 4.83 (m, 1H), 4.61 (m, 1H), 4.54 (m, 1H), 4.34-4.28 (m, 4H), 3.39 (s, 4H), 2.92-2.89 (m, 1H), 2.75-2.70 (m, 1H), 2.62-2.61 (m, 1H), 2.45-2.41 (m, 1H), 2.30-2.18 (m, 1H), 2.15-1.98 (m, 3H), 1.75-1.60 (m, 2H).

### 4-[[1-(2,6-dioxo-3-piperidyl)-3-methyl-2-oxo-benzimidazol-5-yl]amino]-N-[5-fluoro-7-hydroxy-6-(1,1,4-trioxo-1,2,5-thiadiazolidin-2-yl)-2-naphthyl]benzamide (Example 104)

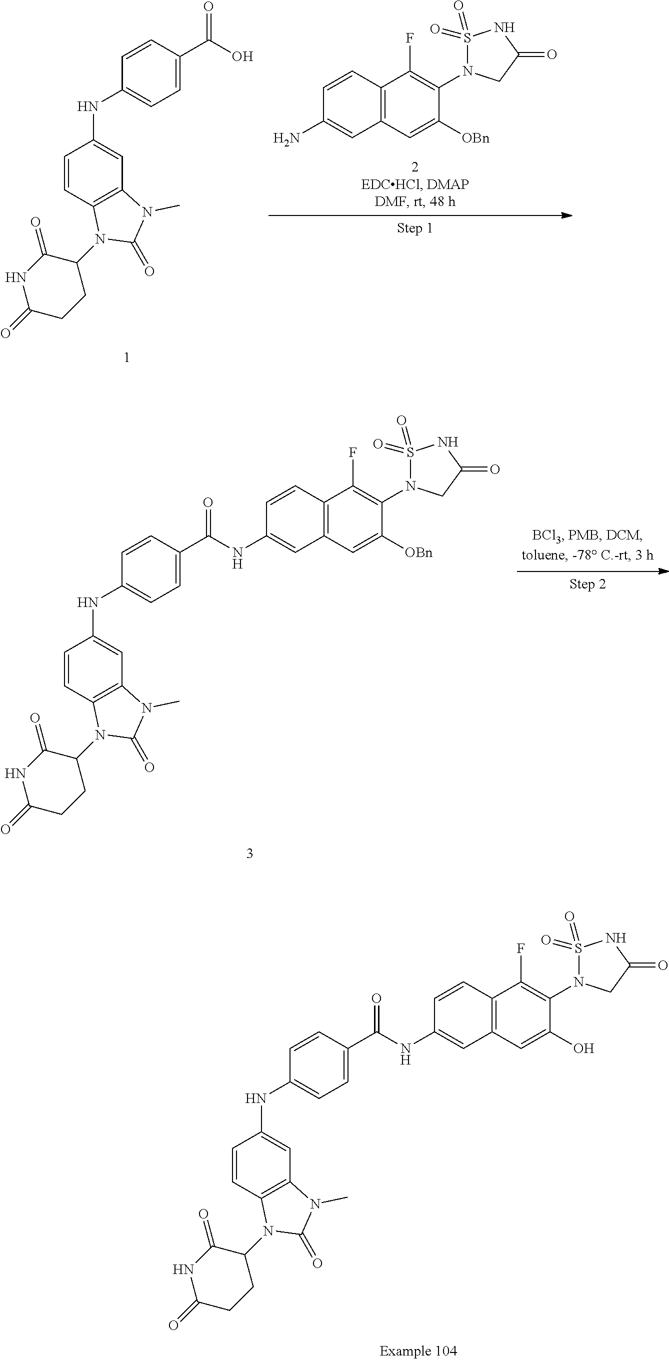 [SMILES: Cn1c(=O)n(C2CCC(=O)NC2=O)c2ccc(Nc3ccc(C(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(Nc3ccc(C(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(Nc3ccc(C(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-4-[[1-(2,6-dioxo-3-piperidyl)-3-methyl-2-oxo-benzimidazol-5-yl]amino]benzamide (3)

Into a 25 mL round bottom flask containing a well-stirred solution of 4-[[1-(2,6-dioxo-3-piperidyl)-3-methyl-2-oxo-benzimidazol-5-yl]amino]benzoic acid (1, 120 mg, 226.59 μmol) in DMF (1 mL) was added EDC hydrochloride (52.13 mg, 271.91 μmol) and 4-dimethylaminopyridine (138.41 mg, 1.13 mmol). After 2 h, 5-(6-amino-3-benzyloxy-1-fluoro-2-naphthyl)-1,1-dioxo-1,2,5-thiadiazolidin-3-one (2, 121.66 mg, 226.59 μmol) was added. After 48 h. the solvent was removed under reduced pressure and the residue was purified by reverse phase column chromatography [Purification method: C18 column; Mobile phase A: 0.1% TFA in water; Mobile phase B: Acetonitrile] to afford N-[7-benzyloxy-5-fluoro-6-(1,1,4-trioxo-1,2,5-thiadiazolidin-2-yl)-2-naphthyl]-4-[[1-(2,6-dioxo-3-piperidyl)-3-methyl-2-oxo-benzimidazol-5-yl]amino]benzamide (3, 80 mg, 84.91 μmol, 37% yield, TFA salt) as a green solid. LCMS (ES+): m/z 778.0 [M+H]⁺

### Step 2: 4-[[1-(2,6-dioxo-3-piperidyl)-3-methyl-2-oxo-benzimidazol-5-yl]amino]-N-[5-fluoro-7-hydroxy-6-(1,1,4-trioxo-1,2,5-thiadiazolidin-2-yl)-2-naphthyl]benzamide (Example 104)

Into a 25 mL round bottom flask containing a well-stirred solution of N-[7-benzyloxy-5-fluoro-6-(1,1,4-trioxo-1,2,5-thiadiazolidin-2-yl)-2-naphthyl]-4-[[1-(2,6-dioxo-3-piperidyl)-3-methyl-2-oxo-benzimidazol-5-yl]amino]benzamide (3, 160 mg, 194.71 μmol) in toluene (2 mL) and DCM (2 mL) was added pentamethyl benzene (144.33 mg, 973.55 μmol, 157.39 μL) and the reaction mixture was cooled to −78° C. Then, boron trichloride (1.0 M in DCM) (456.28 mg, 3.89 mmol, 4.1 mL) was added dropwise over a period of 2 min, subsequently the reaction mixture was stirred at RT. After 3 h the reaction mixture was cooled to −78° C. and quenched with 10% DCM in methanol (2 mL). The reaction mixture was concentrated under reduced pressure at 30° C. The residue was triturated with diethyl ether (10 mL), filtered and purified by reverse phase prep HPLC [Purification method: Column: X-Select, C18 (150×19) mm, 5 micron; Mobile phase A: 0.1% TFA in water and Mobile phase B: MeCN] to afford 4-[[1-(2,6-dioxo-3-piperidyl)-3-methyl-2-oxo-benzimidazol-5-yl]amino]-N-[5-fluoro-7-hydroxy-6-(1,1,4-trioxo-1,2,5-thiadiazolidin-2-yl)-2-naphthyl]benzamide (Example 104, 73 mg, 87.91 μmol, 45% yield, TFA salt) as a yellow solid. LCMS (ES+): m/z 688.0 [M+H]⁺

¹H NMR (400 MHZ, DMSO-d6): δ 11.13 (s, 1H), 10.56 (s, 1H), 10.16 (s, 1H), 8.60 (s, 1H), 8.33 (s, 1H), 7.90 (dd, J=2.80, 9.20 Hz, 1H), 7.11-7.01 (m, 5H), 6.89 (dd, J=2.00, 8.40 Hz, 1H), 5.39-5.35 (m, 1H), 4.48 (s, 2H), 3.34 (s, 3H), 2.97-2.88 (m, 1H), 2.89-2.67 (m, 2H), 2.06-2.02 (m, 1H).

### 2-[4-[[1-(2,6-dioxo-3-piperidyl)-3-methyl-2-oxo-benzimidazol-5-yl]amino]phenyl]-N-[5-fluoro-7-hydroxy-6-(1,1,4-trioxo-1,2,5-thiadiazolidin-2-yl)-2-naphthyl]acetamide (Example 105)

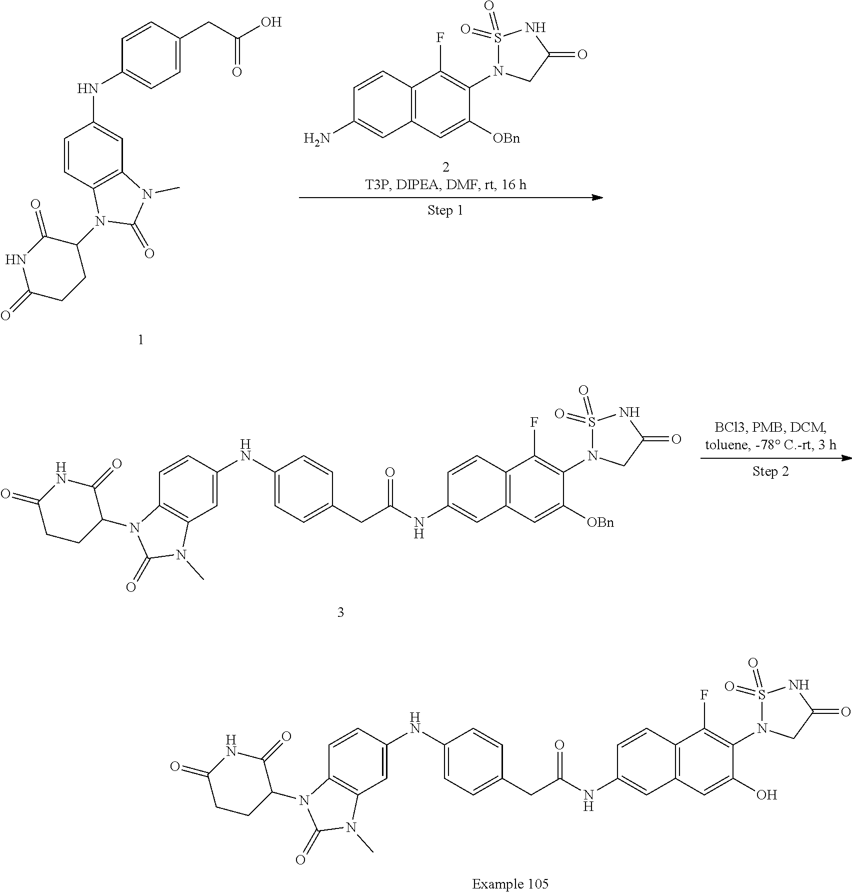 [SMILES: Cn1c(=O)n(C2CCC(=O)NC2=O)c2ccc(Nc3ccc(CC(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(Nc3ccc(CC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(Nc3ccc(CC(=O)Nc4ccc5c(F)c(N6CC(=O)NS6(=O)=O)c(O)cc5c4)cc3)cc21]

### Step 1: N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]amino]phenyl]acetamide (3)

Into a 50 mL single neck round bottom flask containing a well-stirred solution of 2-[4-[[1-(2,6-dioxo-3-piperidyl)-3-methyl-2-oxo-benzimidazol-5-yl]amino]phenyl]acetic acid (1, 218.30 mg, 379.87 μmol, TFA salt) and 5-(6-amino-3-benzyloxy-1-fluoro-2-naphthyl)-1,1-dioxo-1,2,5-thiadiazolidin-3-one (2, 200 mg, 379.87 μmol, TFA salt) in anhydrous DMF (2 mL) was added DIPEA (245.48 mg, 1.90 mmol, 330.84 μL) and T3P (241.60 mg, 759.75 μmol, 0.5 mL). After 20 h, the solvent was removed and the residue was subjected to reverse phase column chromatography [Purification Method: Siliasep premium C18, 120 g column; Mobile phase: 0.1% TFA in Water; Mobile phase B: Acetonitrile] to afford N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]amino]phenyl]acetamide (3, 130 mg, 141.23 μmol, 37% yield, TFA salt) as an off-white solid. LCMS (ES+): m/z 792.3 [M+H]⁺

### Step 2: 2-[4-[[1-(2,6-dioxo-3-piperidyl)-3-methyl-2-oxo-benzimidazol-5-yl]amino]phenyl]-N-[5-fluoro-7-hydroxy-6-(1,1,4-trioxo-1,2,5-thiadiazolidin-2-yl)-2-naphthyl]acetamide (Example 105)

Into a 50 mL two neck round bottom flask containing a well-stirred solution of N-[7-benzyloxy-5-fluoro-6-(1,1,4-trioxo-1,2,5-thiadiazolidin-2-yl)-2-naphthyl]-2-[4-[[1-(2,6-dioxo-3-piperidyl)-3-methyl-2-oxo-benzimidazol-5-yl]amino]phenyl]acetamide (3, 130 mg, 161.57 μmol) in anhydrous DCM (3 mL) and anhydrous toluene (3 mL) was added pentamethylbenzene (119.76 mg, 807.86 μmol, 130.60 μL) under nitrogen atmosphere. The reaction mixture was cooled to −78° C. and treated with BCl₃ (1.0 M in DCM) (3.3 mL, 3.23 mmol) via drop wise addition. The reaction mixture was stirred at RT for 3 h. The reaction mixture was quenched with 5% MeOH in DCM (2 mL) at −78° C. The solvent was removed under reduced pressure and the residue was triturated with diethyl ether and purified by reverse phase prep HPLC [Purification method: X-Select, C18 (150×19), 5 micron; Mobile phase: 0.1% TFA in water; Mobile phase B: MeCN] to afford 2-[4-[[1-(2,6-dioxo-3-piperidyl)-3-methyl-2-oxo-benzimidazol-5-yl]amino]phenyl]-N-[5-fluoro-7-hydroxy-6-(1,1,4-trioxo-1,2,5-thiadiazolidin-2-yl)-2-naphthyl]acetamide (Example 105, 65 mg, 78.45 μmol, 49% yield, TFA salt) as an off-white solid. LCMS (ES+): m/z 701.8 [M+H]⁺

¹H NMR (400 MHZ, DMSO-d6): δ 11.09 (s, 1H), 10.61 (s, 1H), 10.37 (s, 1H), 8.19 (s, 1H), 7.88 (d, J=8.80 Hz, 1H), 7.47 (dd, J=2.00, 9.00 Hz, 1H), 7.19 (d, J=8.80 Hz, 2H), 7.00-6.97 (m, 4H), 6.91 (d, J=2.00 Hz, 1H), 6.78-6.75 (m, 1H), 5.34-5.29 (m, 1H), 4.48 (s, 2H), 3.58 (s, 2H), 3.29 (s, 3H), 2.94-2.86 (m, 1H), 2.74-2.60 (m, 2H), 2.03-2.00 (m, 1H).

### N-[[4-[[1-(2,6-dioxo-3-piperidyl)-3-methyl-2-oxo-benzimidazol-5-yl]phenyl]methyl]-5-fluoro-7-hydroxy-6-(1,1,4-trioxo-1,2,5-thiadiazolidin-2-yl)naphthalene-2-carboxamide (Example 106)

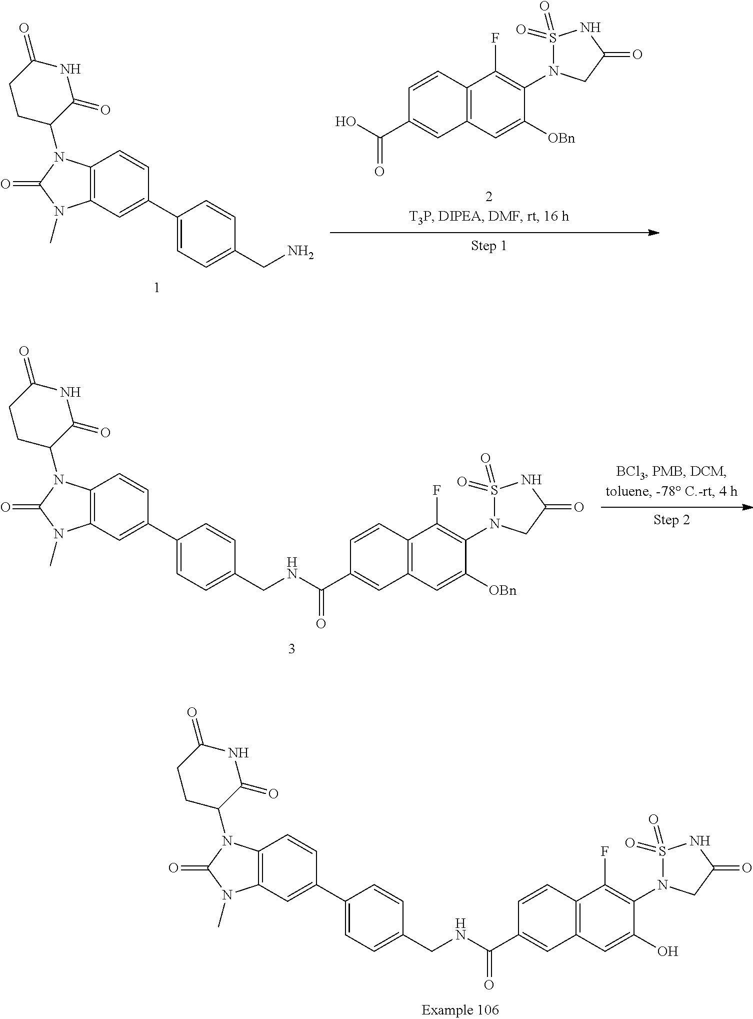 [SMILES: Cn1c(=O)n(C2CCC(=O)NC2=O)c2ccc(-c3ccc(CN)cc3)cc21 ; O=C1CN(c2c(OCc3ccccc3)cc3cc(C(=O)O)ccc3c2F)S(=O)(=O)N1 ; Cn1c(=O)n(C2CCC(=O)NC2=O)c2ccc(-c3ccc(CNC(=O)c4ccc5c(F)c(N6CC(=O)NS6(=O)=O)c(OCc6ccccc6)cc5c4)cc3)cc21 ; Cn1c(=O)n(C2CCC(=O)NC2=O)c2ccc(-c3ccc(CNC(=O)c4ccc5c(F)c(N6CC(=O)NS6(=O)=O)c(O)cc5c4)cc3)cc21]

### Step 1: 7-benzyloxy-N-[[4-[[1-(2,6-dioxo-3-piperidyl)-3-methyl-2-oxo-benzimidazol-5-yl]phenyl]methyl]-5-fluoro-6-(1,1,4-trioxo-1,2,5-thiadiazolidin-2-yl)naphthalene-2-carboxamide (3)

Into a 50 mL single neck round bottom flask containing well-stirred solution of 7-benzyloxy-5-fluoro-6-(1,1,4-trioxo-1,2,5-thiadiazolidin-2-yl)naphthalene-2-carboxylic acid (2, 280 mg, 635.85 μmol) in anhydrous DMF (8 mL) were added propylphosphonic anhydride solution (≥50 wt. % in ethyl acetate) (1.21 mL, 1.91 mmol) and DIPEA (410.89 mg, 3.18 mmol, 553.76 μL). After 15 min, 3-[5-[4-(aminomethyl)phenyl]-3-methyl-2-oxo-benzimidazol-1-yl]piperidine-2,6-dione (1, 353.72 mg, 635.85 μmol, TFA salt) was added. After 16 h, the solvent was removed and the residue purified by reverse phase column chromatography [Purification method: Biotage C18 column; Mobile phase: 0.1% formic acid in water; Mobile phase B: MeCN] to afford 7-benzyloxy-N-[[4-[1-(2,6-dioxo-3-piperidyl)-3-methyl-2-oxo-benzimidazol-5-yl]phenyl]methyl]-5-fluoro-6-(1,1,4-trioxo-1,2,5-thiadiazolidin-2-yl)naphthalene-2-carboxamide (3, 160 mg, 170.94 μmol, 25% yield) as white solid. LCMS (ES+): m/z 777.3 [M+H]⁺

### Step 2: N-[[4-[[1-(2,6-dioxo-3-piperidyl)-3-methyl-2-oxo-benzimidazol-5-yl]phenyl]methyl]-5-fluoro-7-hydroxy-6-(1,1,4-trioxo-1,2,5-thiadiazolidin-2-yl)naphthalene-2-carboxamide (Example 106)

Into a 100 mL single neck round bottom flask containing well-stirred solution of 7-benzyloxy-N-[[4-[1-(2,6-dioxo-3-piperidyl)-3-methyl-2-oxo-benzimidazol-5-yl]phenyl]methyl]-5-fluoro-6-(1,1,4-trioxo-1,2,5-thiadiazolidin-2-yl)naphthalene-2-carboxamide (3, 190 mg, 203.02 μmol) in anhydrous DCM (7.5 mL) and toluene (7.5 mL) was added boron trichloride (1.0 M in DCM) (475.75 mg, 4.06 mmol, 4.06 mL) at −78° C. The reaction mixture was stirred at RT for 4 h. The reaction mixture was quenched with 3 mL of 5% MeOH in DCM at −78° C. and solvents were removed under reduced pressure. The residue was washed with diethyl ether (30 mL) and purified by reverse phase prep HPLC [Purification method: Column: Zorbax C18 (250×21) 7 micron; Mobile phase: 0.1% TFA in water; Mobile phase B: MeCN] to afford N-[[4-[[1-(2,6-dioxo-3-piperidyl)-3-methyl-2-oxo-benzimidazol-5-yl]phenyl]methyl]-5-fluoro-7-hydroxy-6-(1,1,4-trioxo-1,2,5-thiadiazolidin-2-yl)naphthalene-2-carboxamide (Example 106, 85 mg, 119.10 μmol, 59% yield) as white solid. LCMS (ES−): m/z 684.8 [M−H]⁻

¹H NMR (400 MHZ, DMSO-d6): δ 11.14 (s, 1H), 10.90 (s, 1H), 9.28 (t, J=6.00 Hz, 1H), 8.35 (s, 1H), 8.02 (d, J=8.40 Hz, 1H), 7.87 (dd, J=1.60, 8.60 Hz, 1H), 7.68 (d, J=8.00 Hz, 2H), 7.50 (d, J=1.20 Hz, 1H), 7.45 (d, J=8.40 Hz, 2H), 7.35 (dd, J=1.60, 8.40 Hz, 1H), 7.24-7.18 (m, 3H), 5.42-5.38 (m, 1H), 4.58-4.54 (m, 4H), 3.41 (s, 3H), 2.93-2.88 (m, 1H), 2.76-2.63 (m, 2H), 2.09-2.04 (m, 1H).

### 3-(5-(1-(2-((3-(7-(1,1-dioxido-4-oxo-1,2,5-thiadiazolidin-2-yl)-8-fluoro-6-hydroxynaphthalen-2-yl)-2,5-dihydro-1H-pyrrol-1-yl)sulfonyl)ethyl)piperidin-4-yl)-3-methyl-2-oxo-2,3-dihydro-1H-benzo[d]imidazol-1-yl)piperidine-2,6-dione (Example 107)

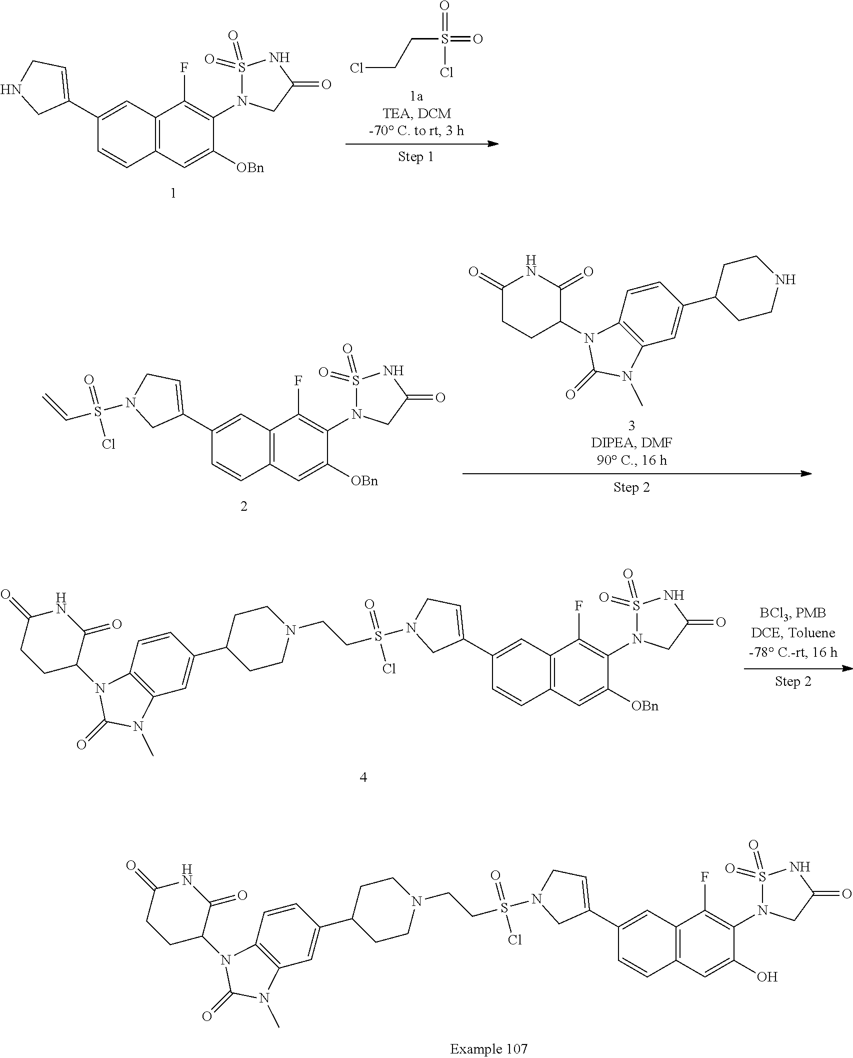

### Step 1: 5-(3-(benzyloxy)-1-fluoro-7-(1-(vinylsulfonyl)-2,5-dihydro-1H-pyrrol-3-yl)naphthalen-2-yl)-1,2,5-thiadiazolidin-3-one 1,1-dioxide (2)

Into a 100 mL single neck round bottom flask containing a well-stirred solution of 5-(3-(benzyloxy)-7-(2,5-dihydro-1H-pyrrol-3-yl)-1-fluoronaphthalen-2-yl)-1,2,5-thiadiazolidin-3-one 1,1-dioxide (1, 700 mg, 1.22 mmol, TFA salt) in DCM (20 mL) was added triethylamine (739.97 mg, 7.31 mmol, 1.02 mL). The reaction mixture was cooled to −70° C. and treated with 2-chloroethanesulfonyl chloride (1a, 596.07 mg, 3.66 mmol, 384.56 μL). After 3 h at RT, the reaction mixture was quenched with water (10 mL) and the aqueous layer was extracted with DCM (2×50 mL). The combined organic layer was washed with 1.5 N HCl solution (15 mL), brine solution (15 mL), dried over anhydrous sodium sulfate and filtered. The solvent was removed under reduced pressure and the residue was purified by reverse phase column chromatography [Purification method: Biotage C18 Column: Mobile phase A: 0.1% TFA in water; Mobile phase B: MeCN] to afford 5-(3-(benzyloxy)-1-fluoro-7-(1-(vinylsulfonyl)-2,5-dihydro-1H-pyrrol-3-yl)naphthalen-2-yl)-1,2,5-thiadiazolidin-3-one 1,1-dioxide (2, 300 mg, 513.37 μmol, 42% yield) as a brown solid. LCMS (ES+): m/z 543.8 [M+H]⁺

### Step 2: 3-(5-(1-(2-((3-(6-(benzyloxy)-7-(1,1-dioxido-4-oxo-1,2,5-thiadiazolidin-2-yl)-8-fluoronaphthalen-2-yl)-2,5-dihydro-1H-pyrrol-1-yl)sulfonyl)ethyl)piperidin-4-yl)-3-methyl-2-oxo-2,3-dihydro-1H-benzo[d]imidazol-1-yl)piperidine-2,6-dione (4)

Into a 10 mL pressure tube containing a well-stirred solution of 3-(3-methyl-2-oxo-5-(piperidin-4-yl)-2,3-dihydro-1H-benzo[d]imidazol-1-yl)piperidine-2,6-dione (3, 111.99 mg, 241.47 μmol, TFA salt) and 5-(3-(benzyloxy)-1-fluoro-7-(1-(vinylsulfonyl)-2,5-dihydro-1H-pyrrol-3-yl)naphthalen-2-yl)-1,2,5-thiadiazolidin-3-one 1,1-dioxide (2, 170.00 mg, 290.91 μmol) in anhydrous DMF (3 mL) was added DIPEA (112.79 mg, 872.73 μmol, 152.01 μL). The reaction mixture was stirred at 90° C. After 16 h, the solvent was removed and the residue was purified by reverse phase column chromatography [Purification method: Biotage-C18 column; Mobile phase A: 0.1% TFA in water; Mobile phase B: MeCN) to afford 3-(5-(1-(2-((3-(6-(benzyloxy)-7-(1,1-dioxido-4-oxo-1,2,5-thiadiazolidin-2-yl)-8-fluoronaphthalen-2-yl)-2,5-dihydro-1H-pyrrol-1-yl)sulfonyl)ethyl)piperidin-4-yl)-3-methyl-2-oxo-2,3-dihydro-1H-benzo[d]imidazol-1-yl)piperidine-2,6-dione (4, 160 mg, 177.29 μmol, 61% yield) as an off-white solid. LCMS (ES+): m/z 886.3 [M+H]⁺

### Step 3: 3-(5-(1-(2-((3-(7-(1,1-dioxido-4-oxo-1,2,5-thiadiazolidin-2-yl)-8-fluoro-6-hydroxynaphthalen-2-yl)-2,5-dihydro-1H-pyrrol-1-yl)sulfonyl)ethyl)piperidin-4-yl)-3-methyl-2-oxo-2,3-dihydro-1H-benzo[d]imidazol-1-yl)piperidine-2,6-dione (Example 107)

Into a 100 mL single neck round bottom flask containing a well-stirred solution of 3-(5-(1-(2-((3-(6-(benzyloxy)-7-(1,1-dioxido-4-oxo-1,2,5-thiadiazolidin-2-yl)-8-fluoronaphthalen-2-yl)-2,5-dihydro-1H-pyrrol-1-yl)sulfonyl)ethyl)piperidin-4-yl)-3-methyl-2-oxo-2,3-dihydro-1H-benzo[d]imidazol-1-yl)piperidine-2,6-dione (4, 170 mg, 188.37 μmol) and penta methylbenzene (167.55 mg, 1.13 mmol, 182.71 μL) in anhydrous toluene (2.5 mL) and DCM (2.5 mL) was added boron trichloride (1.0 M in DCM) (1.88 mL) (220.71 mg, 1.88 mmol) at −78° C. The reaction mixture was then stirred at RT. After 3 h. the reaction mixture was quenched with 3 mL of 5% MeOH in DCM at −78° C. and the volatiles were removed under reduced pressure. The residue was washed with diethyl ether (30 mL) and purified by reverse phase prep HPLC [Purification method: Column: X-Selectc18 (150×19), 5 micron; Mobile phase A: 0.1% TFA in water; Mobile phase B: MeCN] to afford the 3-(5-(1-(2-((3-(7-(1,1-dioxido-4-oxo-1,2,5-thiadiazolidin-2-yl)-8-fluoro-6-hydroxynaphthalen-2-yl)-2,5-dihydro-1H-pyrrol-1-yl)sulfonyl)ethyl)piperidin-4-yl)-3-methyl-2-oxo-2,3-dihydro-1H-benzo[d]imidazol-1-yl)piperidine-2,6-dione (Example 107, 49 mg, 47.92 μmol, 25% yield, TFA salt) as a colorless solid. LCMS (ES+): m/z 796.2 [M+H]⁺

¹H NMR (400 MHZ, DMSO-d6): δ 11.10 (s, 1H), 10.15 (s, 1H), 9.41 (s, 1H), 7.83-7.72 (m, 2H), 7.10-7.05 (m, 3H), 6.92-6.90 (m, 1H), 6.59 (s, 1H), 5.38-5.33 (m, 1H), 4.73 (s, 2H), 4.43 (s, 2H), 4.17 (s, 2H), 3.78-3.60 (m, 6H), 3.34 (s, 3H), 3.19-3.14 (m, 2H), 2.91-2.85 (m, 2H), 2.72-2.65 (m, 2H), 2.09-2.05 (m, 3H), 2.00-1.84 (m, 2H).

### 2-[4-[4-[(2,6-dioxo-3-piperidyl)-methyl-amino]phenyl]-1-piperidyl]-N-[5-fluoro-7-hydroxy-6-(1,1,4-trioxo-1,2,5-thiadiazolidin-2-yl)-2-naphthyl]acetamide (Example 108)

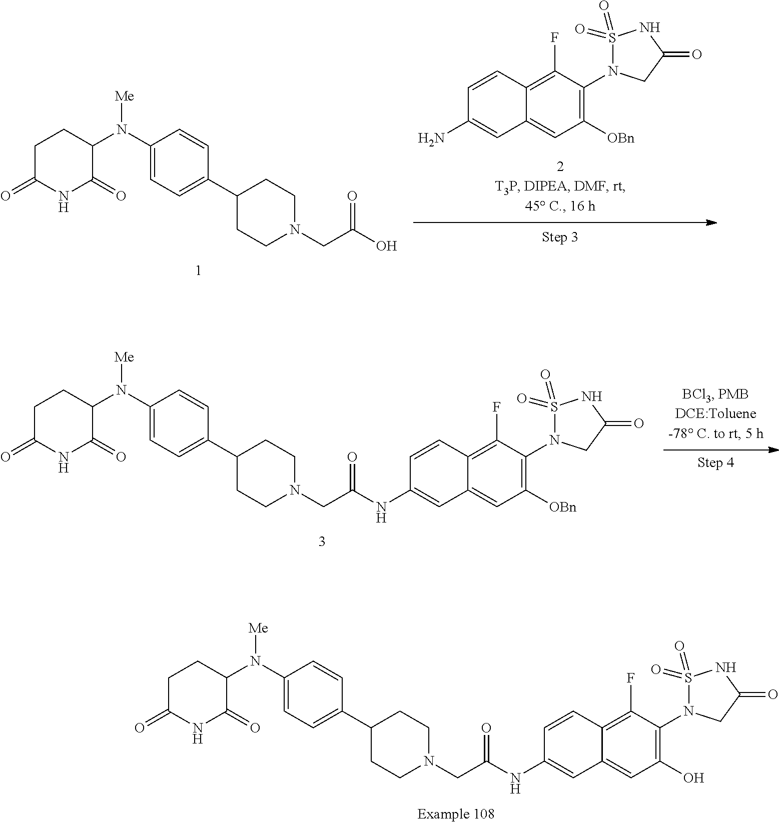 [SMILES: CN(c1ccc(C2CCN(CC(=O)O)CC2)cc1)C1CCC(=O)NC1=O ; Nc1ccc2c(F)c(N3CC(=O)NS3(=O)=O)c(OCc3ccccc3)cc2c1 ; CN(c1ccc(C2CCN(CC(=O)Nc3ccc4c(F)c(N5CC(=O)NS5(=O)=O)c(OCc5ccccc5)cc4c3)CC2)cc1)C1CCC(=O)NC1=O ; CN(c1ccc(C2CCN(CC(=O)Nc3ccc4c(F)c(N5CC(=O)NS5(=O)=O)c(O)cc4c3)CC2)cc1)C1CCC(=O)NC1=O]

### Step 1: N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)-methyl-amino]phenyl]-1-piperidyl]acetamide (3)

Into a 25 mL single neck round bottom flask containing a well-stirred solution of 2-[4-[4-[(2,6-dioxo-3-piperidyl)-methyl-amino]phenyl]-1-piperidyl]acetic acid (1, 150 mg, 313.01 μmol) in anhydrous DMF (3 mL) was added DIPEA (40.45 mg, 313.01 μmol, 54.52 μL) and propylphosphonic anhydride solution ≥50 wt. % in ethyl acetate (199.18 mg, 313.01 μmol, 50% purity) at RT and the resultant mixture was stirred for 10 min at RT. Then 5-(6-amino-3-benzyloxy-1-fluoro-2-naphthyl)-1,1-dioxo-1,2,5-thiadiazolidin-3-one (2, 136.57 mg, 313.01 μmol) was added. The reaction mixture was stirred at RT. After 16 h the volatiles were removed and the resultant crude residue was purified by reverse phase column chromatography [Method: Silycycle C18 (150×19) mm 5 micron column; Mobile phase A: 0.1% formic acid in water; Mobile phase B: MeCN] to afford N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)-methyl-amino]phenyl]-1-piperidyl]acetamide (3, 160 mg, 56.38% yield, formic acid salt). LCMS (ES+): m/z 743.2 [M+H]⁺

### Step 2: 2-[4-[4-[(2,6-dioxo-3-piperidyl)-methyl-amino]phenyl]-1-piperidyl]-N-[5-fluoro-7-hydroxy-6-(1,1,4-trioxo-1,2,5-thiadiazolidin-2-yl)-2-naphthyl]acetamide (Example 108)

Into a 25 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)-methyl-amino]phenyl]-1-piperidyl]acetamide (3, 160 mg, 176.46 μmol) in a mixture of anhydrous toluene (1 mL) and 1,2-DCE (2 mL) was added pentamethyl benzene (130.80 mg, 882.31 μmol) followed by boron trichloride (1.0 M in DCM) (3.53 mL, 3.53 mmol) at −78° C. After 5 h at RT, the mixture was quenched with 3% MeOH in DCM (4 mL) of at −78° C. The volatiles were removed under reduced pressure and the residue was triturated with diethyl ether (10 mL×2). The material was filtered and purified by reverse phase prep HPLC [Purification metho: Zorbax C18 (250×21), 7 micron; Mobile phase A: 10 mM ammonium acetate in water; Mobile phase B: MeCN] to afford 2-[4-[4-[(2,6-dioxo-3-piperidyl)-methyl-amino]phenyl]-1-piperidyl]-N-[5-fluoro-7-hydroxy-6-(1,1,4-trioxo-1,2,5-thiadiazolidin-2-yl)-2-naphthyl]acetamide (Example 108, 55 mg, 47% yield) as a brown solid. LCMS (ES+): m/z 653.2 [M+H]⁺

¹H NMR (400 MHZ, DMSO-d₆): δ 10.80 (s, 1H), 10.78 (s, 1H), 9.84 (bs, 1H), 9.80 (s, 1H), 8.12 (s, 1H), 7.91 (d, J=9.20 Hz, 1H), 7.47 (d, J=9.20 Hz, 1H), 7.08-7.01 (m, 3H), 6.80 (d, J=8.80 Hz, 2H), 4.86 (dd, J=5.20, 12.60 Hz, 1H), 4.28-4.18 (m, 1H), 4.09 (s, 2H), 3.70-3.60 (m, 2H), 3.30-3.19 (m, 2H), 2.88-2.73 (m, 1H), 2.68 (s, 3H), 2.61-2.57 (m, 1H), 2.34-2.29 (m, 1H), 2.02-1.89 (m, 5H).

### 3-[5-[4-[1-[2-[[8-fluoro-6-hydroxy-7-(1,1,4-trioxo-1,2,5-thiadiazolidin-2-yl)-2-naphthyl]oxy]acetyl]-4-piperidyl]-1-piperidyl]-3-methyl-2-oxo-benzimidazol-1-yl]piperidine-2,6-dione (Example 109)

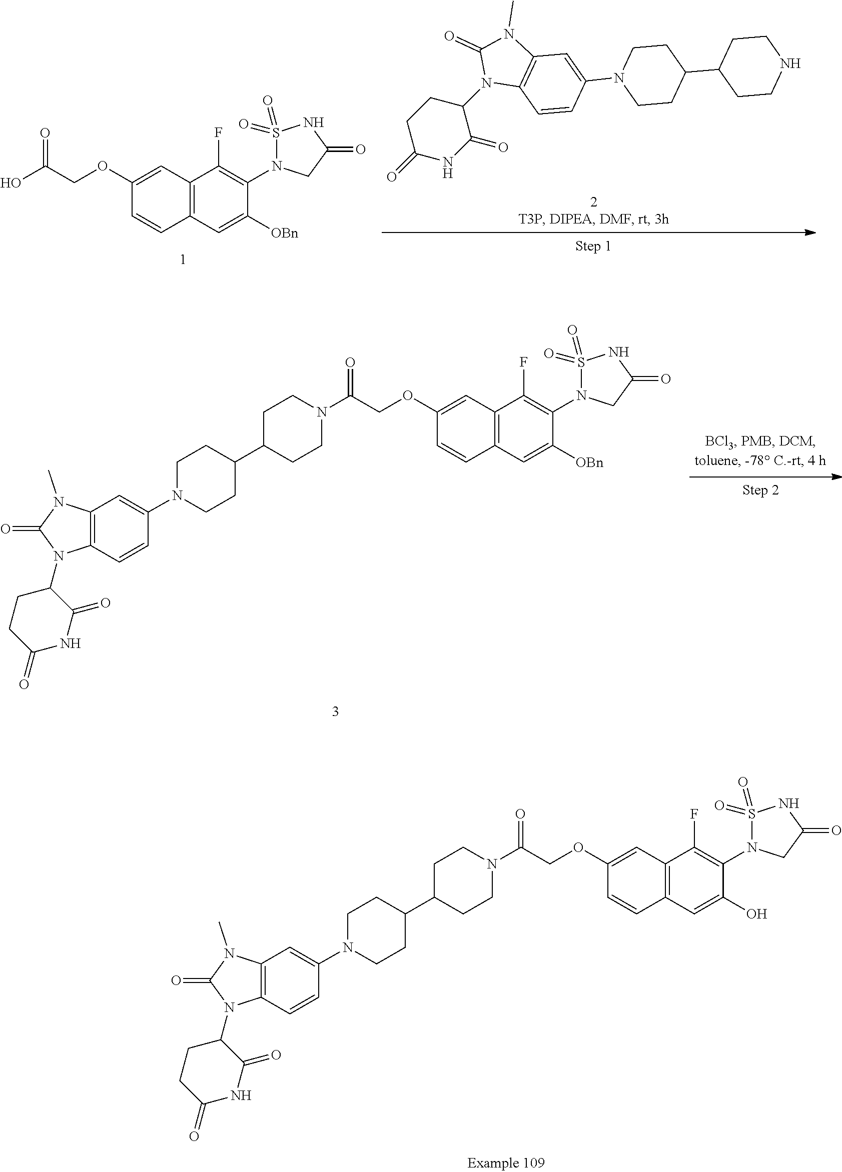

### Step 1: 3-[5-[4-[1-[2-[[6-benzyloxy-8-fluoro-7-(1,1,4-trioxo-1,2,5-thiadiazolidin-2-yl)-2-naphthyl]oxy]acetyl]-4-piperidyl]-1-piperidyl]-3-methyl-2-oxo-benzimidazol-1-yl]piperidine-2,6-dione (3)

Into a 50 mL single neck round bottom flask containing well-stirred solution of 2-[[6-benzyloxy-8-fluoro-7-(1,1,4-trioxo-1,2,5-thiadiazolidin-2-yl)-2-naphthyl]oxy]acetic acid (1, 200 mg, 420.82 μmol) in anhydrous DMF (8 mL) were added propylphosphonic anhydride (≥50 wt. % in ethyl acetate) (1071.18 mg, 1.68 mmol, 1.1 mL, 50% purity) and DIPEA (445.20 mg, 3.44 mmol, 0.6 mL). After 10 min, 3-[3-methyl-2-oxo-5-[4-(4-piperidyl)-1-piperidyl]benzimidazol-1-yl]piperidine-2,6-dione (2, 238.76 mg, 331.89 μmol, TFA salt) was added. After 3 h. the solvent was removed from the reaction mixture under reduced pressure and water (25 mL) was added. The precipitate was filtered and dried under reduced pressure to afford 3-[5-[4-[1-[2-[[6-benzyloxy-8-fluoro-7-(1,1,4-trioxo-1,2,5-thiadiazolidin-2-yl)-2-naphthyl]oxy]acetyl]-4-piperidyl]-1-piperidyl]-3-methyl-2-oxo-benzimidazol-1-yl]piperidine-2,6-dione (3, 220 mg, 224.17 μmol, 53% yield) as an off-white solid. LCMS (ES+): m/z 868.3 [M+H]⁺

### Step 2: 3-[5-[4-[1-[2-[[8-fluoro-6-hydroxy-7-(1,1,4-trioxo-1,2,5-thiadiazolidin-2-yl)-2-naphthyl]oxy]acetyl]-4-piperidyl]-1-piperidyl]-3-methyl-2-oxo-benzimidazol-1-yl]piperidine-2,6-dione (Example 109)

Into a 100 mL single neck round bottom flask containing well-stirred solution of 3-[5-[4-[1-[2-[[6-benzyloxy-8-fluoro-7-(1,1,4-trioxo-1,2,5-thiadiazolidin-2-yl)-2-naphthyl]oxy]acetyl]-4-piperidyl]-1-piperidyl]-3-methyl-2-oxo-benzimidazol-1-yl]piperidine-2,6-dione (3, 220 mg, 223.06 μmol), pentamethylbenzene (165.34 mg, 1.12 mmol) in anhydrous DCM (7.5 mL) and Toluene (7.5 mL) was added boron trichloride (1.0 M in DCM) (522.71 mg, 4.46 mmol, 4.46 mL) at −78° C. The reaction mixture was stirred at RT for 4 h. The reaction was quenched with 5% MeOH in DCM (3 mL) at −78° C. The solvents were removed under reduced pressure and the residue was triturated with diethyl ether and filtered. The material was purified by reverse phase prep HPLC [Purification method: Column: X-Select C18 (150×19) 5 micron; Mobile phase A: 0.1% TFA in water; Mobile phase B: MeCN] to obtain 3-[5-[4-[1-[2-[[8-fluoro-6-hydroxy-7-(1,1,4-trioxo-1,2,5-thiadiazolidin-2-yl)-2-naphthyl]oxy]acetyl]-4-piperidyl]-1-piperidyl]-3-methyl-2-oxo-benzimidazol-1-yl]piperidine-2,6-dione (Example 109, 120 mg, 132.17 μmol, 59% yield, TFA salt) as a colorless solid. LCMS (ES−): m/z 775.8 [M−H]⁻

1H NMR (400 MHZ, DMSO-d6): δ 11.15 (s, 1H), 9.85 (s, 1H), 7.71 (d, J=8.80 Hz, 1H), 7.65 (s, 1H), 7.27-7.21 (m, 2H), 7.20 (dd, J=2.40, 9.00 Hz, 1H), 7.12 (d, J=2.40 Hz, 1H), 7.06 (s, 1H), 5.45-5.40) (m, 1H), 5.05 (d, J=14.80 Hz, 1H), 4.87 (d, J=14.80 Hz, 1H), 4.46-4.43 (m, 1H), 4.27 (s, 2H), 4.05-4.03 (m, 2H), 3.78-3.55 (m, 5H), 3.38 (s, 3H), 3.07-2.92 (m, 2H), 2.74-2.62 (m, 2H), 2.04-1.96 (m, 3H), 1.80-1.78 (m, 2H), 1.74-1.40 (m, 4H), 1.40-1.20 (m, 1H), 1.11-1.00 (m, 1H).

### 3-[5-[4-[2-[4-[8-fluoro-6-hydroxy-7-(1,1,4-trioxo-1,2,5-thiadiazolidin-2-yl)-2-naphthyl]pyrazol-1-yl]ethyl]piperazin-1-yl]-3-methyl-2-oxo-benzimidazol-1-yl]piperidine-2,6-dione (Example 110)

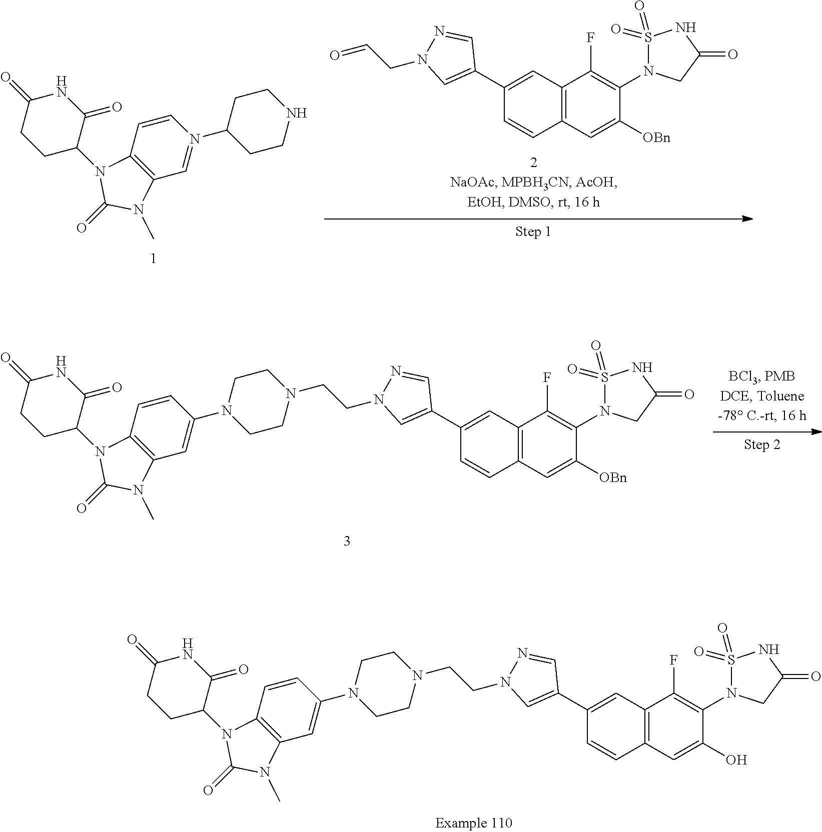

### Step 1: 3-[5-[4-[2-[4-[6-benzyloxy-8-fluoro-7-(1,1,4-trioxo-1,2,5-thiadiazolidin-2-yl)-2-naphthyl]pyrazol-1-yl]ethyl]piperazin-1-yl]-3-methyl-2-oxo-benzimidazol-1-yl]piperidine-2,6-dione (3)

Into a 25 mL single neck round bottom flask containing a well-stirred solution of 3-(3-methyl-2-oxo-5-piperazin-1-yl-benzimidazol-1-yl)piperidine-2,6-dione (1, 120 mg, 236.12 μmol, TFA salt) and 2-[4-[6-benzyloxy-8-fluoro-7-(1,1,4-trioxo-1,2,5-thiadiazolidin-2-yl)-2-naphthyl]pyrazol-1-yl]acetaldehyde (2, 318.43 mg, 283.34 μmol, 44% purity) in DMSO (2 mL) and ethanol (2 mL) was added sodium acetate (58.11 mg, 708.35 μmol, 37.98 μL) and acetic acid (141.79 mg, 2.36 mmol, 135.04 μL). After 5 min, MP-cyano borohydride (2 mmol/g, 240 mg, 480 μmol) was added and the reaction mixture was stirred at ambient temperature. After 16 h, the reaction mixture was filtered and concentrated under reduced pressure. The residue was subjected to reverse phase column chromatography [Purification method: Biotage C18 column; Mobile phase A: 0.1% TFA in water; Mobile phase B: Acetonitrile) to afford 3-[5-[4-[2-[4-[6-benzyloxy-8-fluoro-7-(1,1,4-trioxo-1,2,5-thiadiazolidin-2-yl)-2-naphthyl]pyrazol-1-yl]ethyl]piperazin-1-yl]-3-methyl-2-oxo-benzimidazol-1-yl]piperidine-2,6-dione (3, 80 mg, 76.93 μmol, 33% yield, TFA salt) as an off-white solid. LCMS (ES+): m/z 822.2 [M+H]⁺

### Step 2: 3-[5-[4-[2-[4-[8-fluoro-6-hydroxy-7-(1,1,4-trioxo-1,2,5-thiadiazolidin-2-yl)-2-naphthyl]pyrazol-1-yl]ethyl]piperazin-1-yl]-3-methyl-2-oxo-benzimidazol-1-yl]piperidine-2,6-dione (Example 110)

Into a 25 mL round bottom flask containing a well-stirred solution of 3-[5-[4-[2-[4-[6-benzyloxy-8-fluoro-7-(1,1,4-trioxo-1,2,5-thiadiazolidin-2-yl)-2-naphthyl]pyrazol-1-yl]ethyl]piperazin-1-yl]-3-methyl-2-oxo-benzimidazol-1-yl]piperidine-2,6-dione (3, 80 mg, 76.93 μmol, TFA salt) in DCM (1.5 mL) and toluene (1.5 mL) was added pentamethylbenzene (57.02 mg, 384.66 μmol, 62.18 μL) and the reaction mixture was cooled to −78° C. Then boron trichloride (1.0 M in DCM) (270.42 mg, 2.31 mmol, 1.3 mL) was added dropwise over a period of 2 min. After 16 h at RT, the reaction mixture was cooled to −78° C. and quenched slowly with 10% methanol in DCM (2 mL). The reaction mixture was concentrated under reduced pressure at 30° C. The residue was washed with diethyl ether (10 mL), filtered and purified by reverse phase prep HPLC [Purification method: Column: X-Select, C18 (150×19) mm, 5 micron; Mobile phase A: 0.1% TFA in water; Mobile phase B: MeCN] to afford 3-[5-[4-[2-[4-[8-fluoro-6-hydroxy-7-(1,1,4-trioxo-1,2,5-thiadiazolidin-2-yl)-2-naphthyl]pyrazol-1-yl]ethyl]piperazin-1-yl]-3-methyl-2-oxo-benzimidazol-1-yl]piperidine-2,6-dione (Example 110, 26 mg, 30.42 μmol, 40% yield, TFA salt) as a colorless solid. LCMS (ES+): m/z 732.2 [M+H]⁺

¹H NMR (400 MHZ, DMSO-d6): δ 11.08 (s, 1H), 10.00 (s, 1H), 9.80 (s, 1H), 8.45 (s, 1H), 8.18 (s, 1H), 8.06 (s, 1H), 7.80-7.74 (m, 2H), 7.07 (s, 1H), 7.00 (d, J=8.80 Hz, 1H), 6.94 (d, J=2.00 Hz, 1H), 6.71 (dd, J=2.00, 8.80 Hz, 1H), 5.33-5.28 (m, 1H), 4.64-4.50 (m, 2H), 4.17 (s, 2H), 3.75-3.61 (m, 4H), 3.32 (s, 3H), 3.00-2.85 (m, 3H), 2.73-2.64 (m, 2H), 2.00-1.98 (m, 1H).

### N-[3-[1-(2,6-dioxo-3-piperidyl)-2-oxo-benzo[cd]indol-6-yl]propyl]-5-[[8-fluoro-6-hydroxy-7-(1,1,4-trioxo-1,2,5-thiadiazolidin-2-yl)-2-naphthyl]oxy]pentanamide (Example 111)

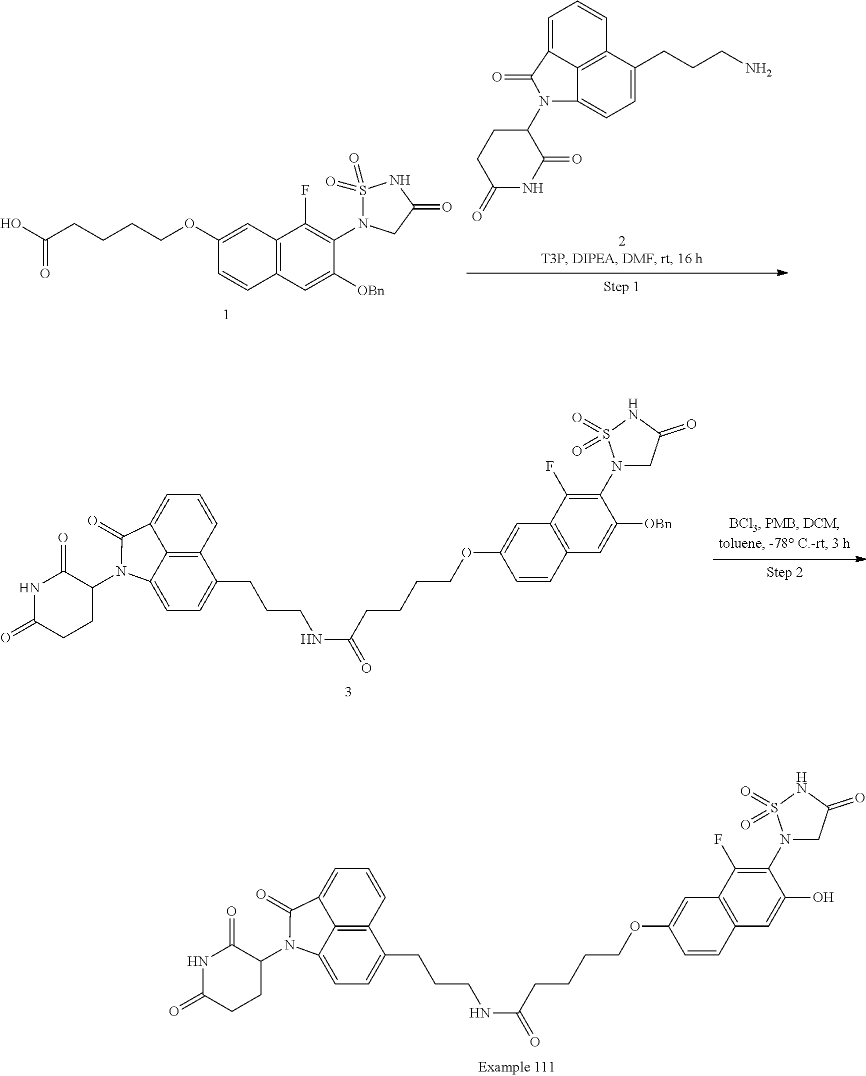

### Step 1: 5-((6-(benzyloxy)-7-(1,1-dioxido-4-oxo-1,2,5-thiadiazolidin-2-yl)-8-fluoronaphthalen-2-yl)oxy)-N-(3-(1-(2,6-dioxopiperidin-3-yl)-2-oxo-1,2-dihydrobenzo[cd]indol-6-yl)propyl)pentanamide (3)

Into a 8 mL vial containing a well-stirred solution of 3-[6-(3-aminopropyl)-2-oxo-benzo[cd]indol-1-yl]piperidine-2,6-dione (2, 0.12 g, 321.00 μmol, HCl salt) and 5-[[6-benzyloxy-8-fluoro-7-(1,1,4-trioxo-1,2,5-thiadiazolidin-2-yl)-2-naphthyl]oxy]pentanoic acid (1, 177.22 mg, 321.00 μmol) in anhydrous DMF (3 mL) was added diisopropylethylamine (165.94 mg, 1.28 mmol) followed by 1-propanephosphonic anhydride (50% in ethyl acetate) (306.41 mg, 481.50 μmol, 50% purity). After 16 h, the reaction mixture was concentrated under reduced pressure and the residue was purified by reverse phase column chromatography [Purification method: Biotage C18 column; Mobile phase A: 0.1% TFA in water; Mobile phase B: Acetonitrile] to afford 5-((6-(benzyloxy)-7-(1,1-dioxido-4-oxo-1,2,5-thiadiazolidin-2-yl)-8-fluoronaphthalen-2-yl)oxy)-N-(3-(1-(2,6-dioxopiperidin-3-yl)-2-oxo-1,2-dihydrobenzo[cd]indol-6-yl)propyl)pentanamide (3, 0.16 g, 171.32 μmol, 53% yield) as a yellow solid. LCMS (ES+): m/z 822.2 [M+H]⁺

### Step 2: N-[3-[1-(2,6-dioxo-3-piperidyl)-2-oxo-benzo[cd]indol-6-yl]propyl]-5-[[8-fluoro-6-hydroxy-7-(1,1,4-trioxo-1,2,5-thiadiazolidin-2-yl)-2-naphthyl]oxy]pentanamide (Example 111)

Into a 25 mL round bottom flask containing a well-stirred solution of 5-((6-(benzyloxy)-7-(1,1-dioxido-4-oxo-1,2,5-thiadiazolidin-2-yl)-8-fluoronaphthalen-2-yl)oxy)-N-(3-(1-(2,6-dioxopiperidin-3-yl)-2-oxo-1,2-dihydrobenzo[cd]indol-6-yl)propyl)pentanamide (3, 181.82 mg, 194.68 μmol) and pentamethylbenzene (5, 144.31 mg, 973.39 μmol) in anhydrous DCM (3 mL) and toluene (3 mL) was added a boron trichloride (1.0 M in DCM) (456.21 mg, 3.89 mmol, 3.9 mL) dropwise at −78° C. The resulting mixture was stirred at RT for 3 h. The reaction was cooled to −78° C. and quenched by dropwise addition of 10% solution of MeOH in DCM (1.5 mL). The volatiles were removed under reduced pressure and the residue purified by reverse phase prep HPLC [Purification method: Column-X Bridge C18 (19×150 mm) 5 micron; Mobile phase A: 0.1% TFA in water; Mobile phase B: Acetonitrile) to obtain N-[3-[1-(2,6-dioxo-3-piperidyl)-2-oxo-benzo[cd]indol-6-yl]propyl]-5-[[8-fluoro-6-hydroxy-7-(1,1,4-trioxo-1,2,5-thiadiazolidin-2-yl)-2-naphthyl]oxy]pentanamide (Example 111, 58.01 mg, 76.40 μmol, 39% yield) as a yellow solid. LCMS (ES+): m/z 732.0 [M+H]⁺

¹HNMR (400 MHZ, DMSO-d6): δ 11.13 (s, 1H), 10.27 (bs, 1H), 8.33 (d, J=8.40 Hz, 1H), 8.09 (d, J=6.80 Hz, 1H), 7.93 (t, J=5.60 Hz, 1H), 7.86-7.82 (m, 1H), 7.69 (d, J=8.80 Hz, 1H), 7.34 (d, J=7.20 Hz, 1H), 7.19-7.15 (m, 2H), 7.08-7.06 (m, 2H), 5.46-5.42 (m, 1H), 4.46 (s, 2H), 4.08 (t, J=6.00 Hz, 2H), 3.17-3.13 (m, 2H), 3.04-2.95 (m, 3H), 2.77-2.63 (m, 3H), 2.19 (t, J=6.80) Hz, 2H), 2.11-2.08 (m, 1H), 1.82-1.72 (m, 6H).

### 3-[5-[1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-3-methyl-2-oxo-benzimidazol-1-yl]piperidine-2,6-dione (Example 112)

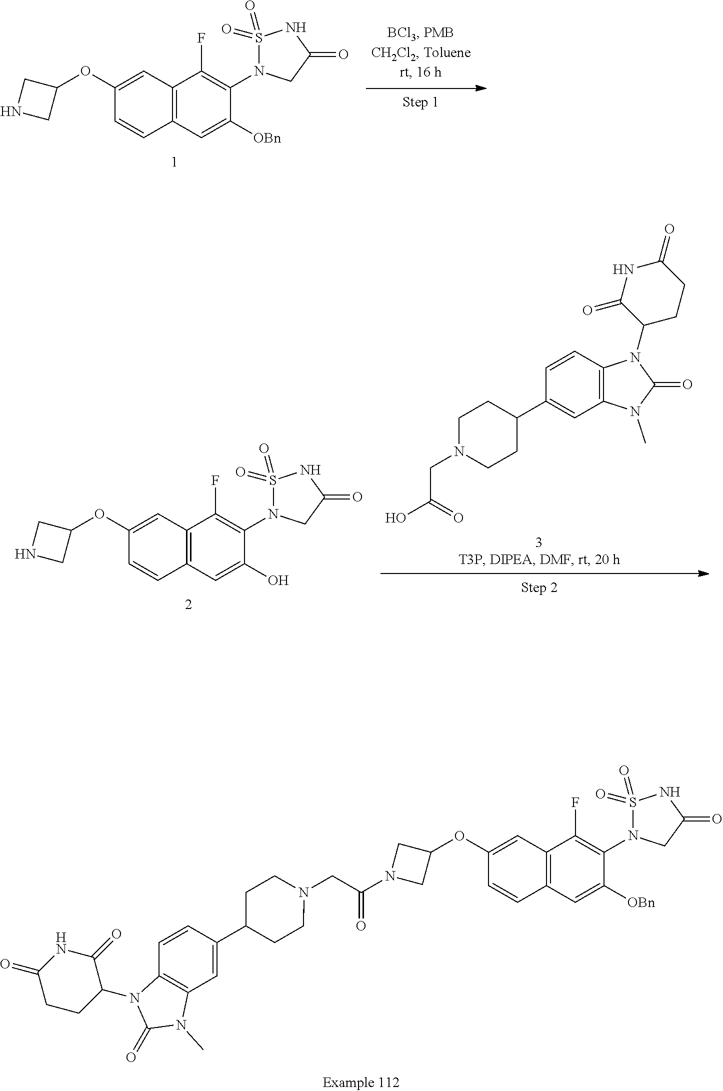 [SMILES: O=C1CN(c2c(OCc3ccccc3)cc3ccc(OC4CNC4)cc3c2F)S(=O)(=O)N1 ; O=C1CN(c2c(O)cc3ccc(OC4CNC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)N4CC(Oc5ccc6cc(OCc7ccccc7)c(N7CC(=O)NS7(=O)=O)c(F)c6c5)C4)CC3)cc21]

### Step 1: 5-[7-(azetidin-3-yloxy)-1-fluoro-3-hydroxy-2-naphthyl]-1,1-dioxo-1,2,5-thiadiazolidin-3-one (2)

Into a 25 mL single neck round bottom flask containing a well-stirred solution of 5-[7-(azetidin-3-yloxy)-3-benzyloxy-1-fluoro-2-naphthyl]-1,1-dioxo-1,2,5-thiadiazolidin-3-one (1, 500 mg, 1.01 μmol, HCl salt) in anhydrous DCM (2.5 mL) and toluene (2.5 mL) was added pentamethylbenzene (900.39 mg, 6.07 μmol). The reaction mixture was cooled to −78° C., then treated with boron trichloride (1.0 M in DCM) (10.12 mmol, 10.12 mL). After 16 h at RT, the reaction was quenched with 5% MeOH in DCM (10 mL) at −78° C. The reaction mixture was concentrated under reduced pressure. The residue was purified by reverse phase purification [Purification method: Column: Biotage C18 (150×19) mm 5 microns; Mobile phase A: 0.1% TFA in water; Mobile phase B: Acetonitrile] to afford 5-[7-(azetidin-3-yloxy)-1-fluoro-3-hydroxy-2-naphthyl]-1,1-dioxo-1,2,5-thiadiazolidin-3-one 2 (230 mg, 43% yield, TFA salt) as a white solid. LCMS (ES+): m/z 368 [M+H]⁺

### Step 2: 3-[5-[1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-3-methyl-2-oxo-benzimidazol-1-yl]piperidine-2,6-dione (Example 112)

Into a 50 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (2, 90 mg, 167.16 μmol, TFA salt) in anhydrous DMF (2 mL) were added DIPEA (64.81 mg, 501.48 μmol, 87.35 μL) and propylphosphonic anhydride (50 wt. % in ethyl acetate) (159.56 mg, 250.74 μmol, 149.12 μL). After 10 min, 5-[7-(azetidin-3-yloxy)-1-fluoro-3-hydroxy-2-naphthyl]-1,1-dioxo-1,2,5-thiadiazolidin-3-one (3, 74.63 mg, 167.16 μmol, HCl salt) was added. After 16 h, the reaction mixture was concentrated under reduced pressure to obtain the crude compound which was purified by using reverse phase PREP HPLC [Purification method: X-select C18 (150×19) mm 5 micron; Mobile phase A: 0.1% TFA water; Mobile phase B: Acetonitrile] to obtain 3-[5-[1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-3-methyl-2-oxo-benzimidazol-1-yl]piperidine-2,6-dione (Example 112, 83.73 mg, 57% yield, TFA salt) as an off-white solid. LCMS (ES+): m/z 750 [M+H]⁺

¹H NMR (400 MHZ, DMSO-d₆): δ 11.11 (s, 1H), 9.84 (s, 1H), 9.70 (s, 1H), 7.77 (d, J=9.20 Hz, 1H), 7.19 (dd, J=2.80, 9.00 Hz, 1H), 7.13-7.04 (m, 3H), 7.00 (d, J=2.40 Hz, 1H), 6.93 (d, J=8.40 Hz, 1H), 5.36 (dd, J=5.60, 13.00 Hz, 1H), 5.33-5.27 (m, 1H), 4.74-4.73 (m, 1H), 4.54-4.53 (m, 1H), 4.26-4.20 (m, 3H), 4.10-4.03 (m, 3H), 3.35-3.34 (m, 4H), 3.15-3.05 (m, 2H), 2.91-2.81 (m, 2H), 2.72-2.62 (m, 2H), 2.07-2.01 (m, 5H).

### 2-[4-[4-[(2,6-dioxo-3-piperidyl)amino]phenyl]-3,3-difluoro-1-piperidyl]-N-[5-fluoro-7-hydroxy-6-(1,1,4-trioxo-1,2,5-thiadiazolidin-2-yl)-2-naphthyl]acetamide (Example 113)

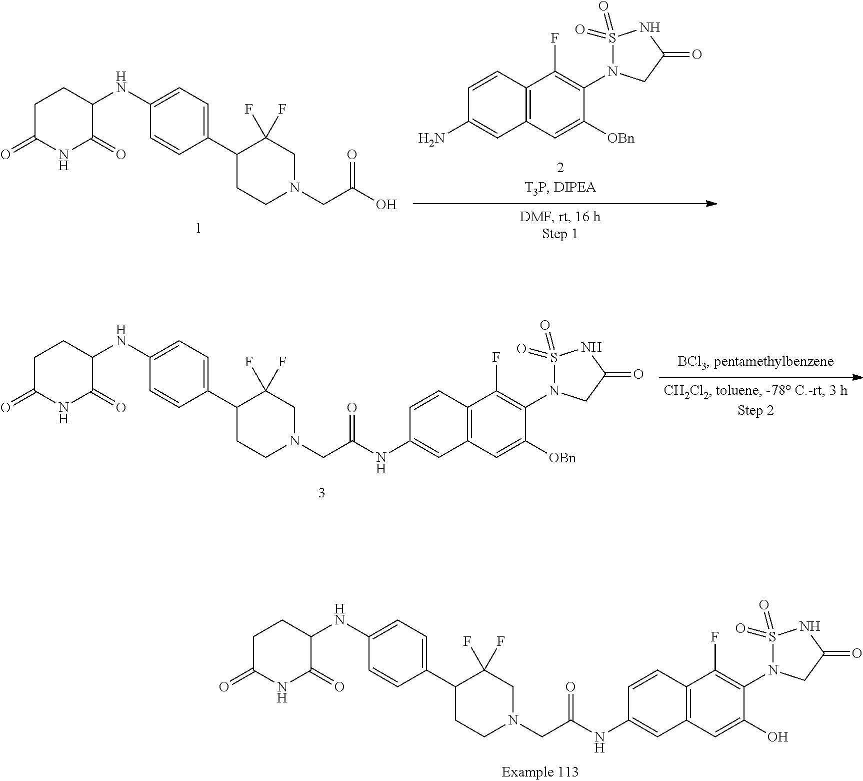 [SMILES: O=C(O)CN1CCC(c2ccc(NC3CCC(=O)NC3=O)cc2)C(F)(F)C1 ; Nc1ccc2c(F)c(N3CC(=O)NS3(=O)=O)c(OCc3ccccc3)cc2c1 ; O=C1CCC(Nc2ccc(C3CCN(CC(=O)Nc4ccc5c(F)c(N6CC(=O)NS6(=O)=O)c(OCc6ccccc6)cc5c4)CC3(F)F)cc2)C(=O)N1 ; O=C1CCC(Nc2ccc(C3CCN(CC(=O)Nc4ccc5c(F)c(N6CC(=O)NS6(=O)=O)c(O)cc5c4)CC3(F)F)cc2)C(=O)N1]

### Step 1: N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)amino]phenyl]-3,3-difluoro-1-piperidyl]acetamide (3)

Into a 20 mL vial containing a well-stirred solution of 2-[4-[4-[(2,6-dioxo-3-piperidyl)amino]phenyl]-3,3-difluoro-1-piperidyl]acetic acid (1, 250 mg, 467.30 μmol, TFA salt) and 5-(6-amino-3-benzyloxy-1-fluoro-2-naphthyl)-1,1-dioxo-1,2,5-thiadiazolidin-3-one (2, 253.73 mg, 467.30 μmol, TFA salt) in DMF (3 mL) was added DIPEA (603.94 mg, 4.67 mmol, 813.94 μL) followed by and propylphosphonic anhydride solution (50% in EtOAc) (594.75 mg, 934.61 μmol). After 16 h, the solvent was removed under reduced pressure and the residue was subjected to reverse-phase column chromatography (120 g of C18 column; 0.1% TFA in water and acetonitrile) to afford N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)amino]phenyl]-3,3-difluoro-1-piperidyl]acetamide (3, 230 mg, 242.75 μmol, 52% yield, TFA salt) as an off-white solid. LCMS (ES+): m/z 765.0 [M+H]⁺

### Step 2: 2-[4-[4-[(2,6-dioxo-3-piperidyl)amino]phenyl]-3,3-difluoro-1-piperidyl]-N-[5-fluoro-7-hydroxy-6-(1,1,4-trioxo-1,2,5-thiadiazolidin-2-yl)-2-naphthyl]acetamide (Example 113)

Into a 25 mL round bottom flask containing a well-stirred solution of N-[7-benzyloxy-5-fluoro-6-(1,1,4-trioxo-1,2,5-thiadiazolidin-2-yl)-2-naphthyl]-2-[4-[4-[(2,6-dioxo-3-piperidyl)amino]phenyl]-3,3-difluoro-1-piperidyl]acetamide (5, 180 mg, 218.30 μmol, TFA salt) in DCM (2.5 mL) and toluene (2.5 mL) was added pentamethylbenzene (161.82 mg, 1.09 mmol, 176.46 μL) and the reaction mixture was cooled to −78° C. Then BCl₃ (1.0 M solution in DCM) (5.99 mmol, 6 mL) was added dropwise over a period of 2 min. The reaction was stirred at RT for 3 h. The reaction mixture was cooled to −78° C. and quenched slowly with 10% methanol in DCM (2 mL). The solvent was removed under reduced pressure at 30° C. and the residue purified by reverse-phase preparative HPLC [Column: X Select C18 (150×30) mm 5 microns; 0.1% TFA in water:CH₃CN] to afford 2-[4-[4-[(2,6-dioxo-3-piperidyl)amino]phenyl]-3,3-difluoro-1-piperidyl]-N-[5-fluoro-7-hydroxy-6-(1,1,4-trioxo-1,2,5-thiadiazolidin-2-yl)-2-naphthyl]acetamide (Example 113, 60 mg, 73.60 μmol, 34% yield, TFA salt) as an off-white solid. LCMS (ES+): m/z 675.2 [M+H]⁺

¹H NMR (400 MHZ, DMSO-d6): δ 10.81 (s, 1H), 10.30 (brs, 1H), 10.20 (brs, 1H), 8.17 (s, 1H), 7.89 (d, J=8.80 Hz, 1H), 7.47 (dd, J=1.60, 9.20 Hz, 1H), 7.04 (d, J=8.40 Hz, 2H), 7.00 (s, 1H), 6.66 (d, J=8.80 Hz, 1H), 4.32 (dd, J=4.80, 11.20 Hz, 1H), 4.27 (s, 2H), 3.95-3.75 (m, 2H), 3.46-3.05 (m, 4H), 2.75-2.60 (m, 3H), 2.34-2.33 (m, 1H), 2.09-1.90 (m, 3H).

### N-(6-(1,1-dioxido-4-oxo-1,2,5-thiadiazolidin-2-yl)-5-fluoro-7-hydroxynaphthalen-2-yl)-2-(4-(3-(2,6-dioxopiperidin-3-yl)-1-methyl-1H-indazol-6-yl)-3,3-difluoropiperidin-1-yl)acetamide (Example 114)

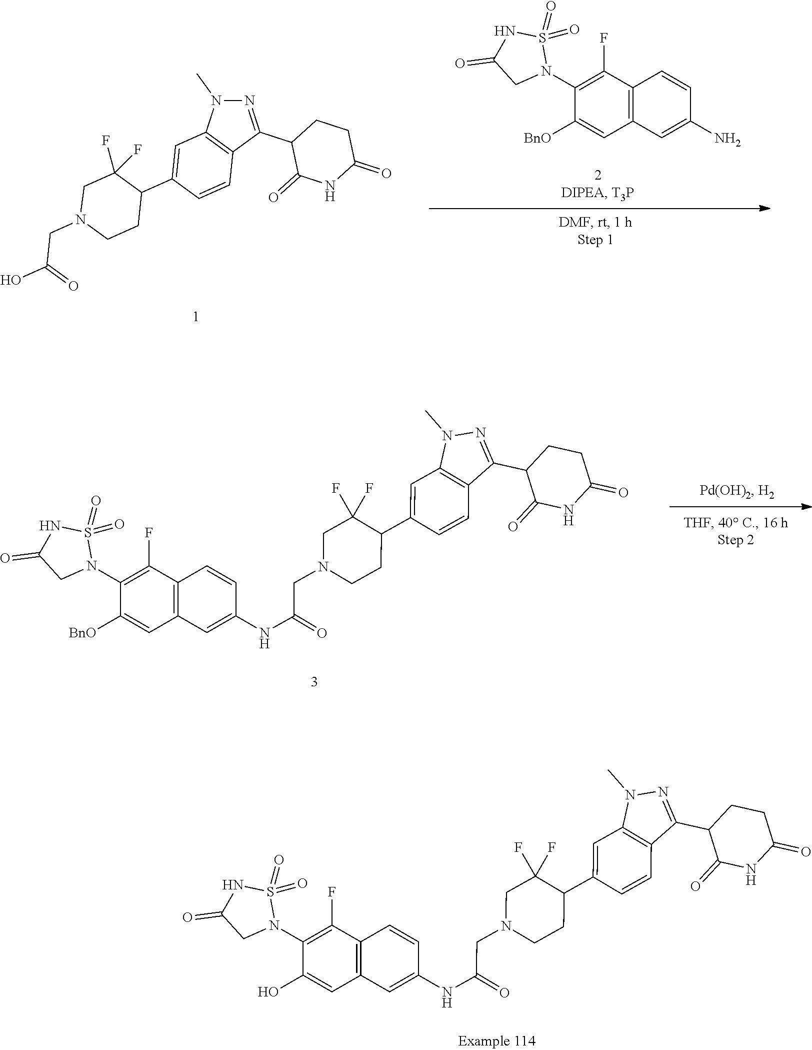 [SMILES: Cn1nc(C2CCC(=O)NC2=O)c2ccc(C3CCN(CC(=O)O)CC3(F)F)cc21 ; Nc1ccc2c(F)c(N3CC(=O)NS3(=O)=O)c(OCc3ccccc3)cc2c1 ; Cn1nc(C2CCC(=O)NC2=O)c2ccc(C3CCN(CC(=O)Nc4ccc5c(F)c(N6CC(=O)NS6(=O)=O)c(OCc6ccccc6)cc5c4)CC3(F)F)cc21 ; Cn1nc(C2CCC(=O)NC2=O)c2ccc(C3CCN(CC(=O)Nc4ccc5c(F)c(N6CC(=O)NS6(=O)=O)c(O)cc5c4)CC3(F)F)cc21]

### Step 1: N-(7-(benzyloxy)-6-(1,1-dioxido-4-oxo-1,2,5-thiadiazolidin-2-yl)-5-fluoronaphthalen-2-yl)-2-(4-(3-(2,6-dioxopiperidin-3-yl)-1-methyl-1H-indazol-6-yl)-3,3-difluoropiperidin-1-yl)acetamide (3)

To a solution of 2-(4-(3-(2,6-dioxopiperidin-3-yl)-1-methyl-1H-indazol-6-yl)-3,3-difluoropiperidin-1-yl)acetic acid (1, 200 mg, 437.76 μmol, HCl salt) in DMF (4 mL) was added T₃P (50% in EtOAc) (334.29 mg, 525.31 μmol, 312.42 μL) and DIPEA (282.89 mg, 2.19 mmol, 381.25 μL). After 1 h, 5-(6-amino-3-(benzyloxy)-1-fluoronaphthalen-2-yl)-1,2,5-thiadiazolidin-3-one 1,1-dioxide (2, 225.64 mg, 437.76 μmol, TFA salt) was added. The mixture was stirred for 16 h. The reaction mixture was diluted with water (4 mL) and extracted with EtOAc (5 mL×3). The combined organic phase was washed with brine (5 mL) and concentrated under reduced pressure. The residue was purified by prep-TLC (DCM/MeOH=10/1) to afford N-(7-(benzyloxy)-6-(1,1-dioxido-4-oxo-1,2,5-thiadiazolidin-2-yl)-5-fluoronaphthalen-2-yl)-2-(4-(3-(2,6-dioxopiperidin-3-yl)-1-methyl-1H-indazol-6-yl)-3,3-difluoropiperidin-1-yl)acetamide (3, 150 mg, 82.11 μmol, 18% yield, 44% purity) as yellow solid. LCMS (ESI): m/z 803.9 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(3-(2,6-dioxopiperidin-3-yl)-1-methyl-1H-indazol-6-yl)-3,3-difluoropiperidin-1-yl)acetamide (Example 114)

To a solution of N-(7-(benzyloxy)-6-(1,1-dioxido-4-oxo-1,2,5-thiadiazolidin-2-yl)-5-fluoronaphthalen-2-yl)-2-(4-(3-(2,6-dioxopiperidin-3-yl)-1-methyl-1H-indazol-6-yl)-3,3-difluoropiperidin-1-yl)acetamide (3, 150 mg, 186.61 μmol) in THF (10 mL) was added Pd(OH)₂ (2.62 mg, 18.66 μmol). The reaction mixture was stirred at 40° C. under H₂ atmosphere for 16 h. The reaction mixture was filtered through Celite. The filtrate was concentrated under reduced pressure and the residue was purified by prep-HPLC (flow: 27 mL/min; gradient: from 13-43% water (0.1% TFA) in MeCN over 10 min; column: Phenomenex Synergi C18 150×25 mm×10 μm) to afford N-(6-(1,1-dioxido-4-oxo-1,2,5-thiadiazolidin-2-yl)-5-fluoro-7-hydroxynaphthalen-2-yl)-2-(4-(3-(2,6-dioxopiperidin-3-yl)-1-methyl-1H-indazol-6-yl)-3,3-difluoropiperidin-1-yl)acetamide (Example 114, 27 mg, 30.66 μmol, 16% yield, TFA salt). LCMS (ESI): m/z 714.2 [M+H]⁺

¹H NMR (400 MHZ, d₆-DMSO) δ 10.91 (s, 1H), 10.32-10.10 (m, 2H), 8.19 (s, 1H), 7.90 (d, J=9.0 Hz, 1H), 7.69 (d, J=8.5 Hz, 1H), 7.57 (s, 1H), 7.50 (dd, J=1.8, 9.0 Hz, 1H), 7.13 (d, J=8.4 Hz, 1H), 7.01 (s, 1H), 4.38 (dd, J=4.9, 9.9 Hz, 1H), 4.31 (s, 2H), 4.02 (s, 3H), 3.52-3.05 (m, 6H), 2.93-2.77 (m, 1H), 2.71-2.61 (m, 2H), 2.47-2.36 (m, 2H), 2.26-2.14 (m, 1H), 2.02-1.90 (m, 1H).

### 3-(5-(1-(2-(3-(6-(1,1-dioxido-4-oxo-1,2,5-thiadiazolidin-2-yl)-5-fluoro-7-hydroxynaphthalen-2-yl)azetidin-1-yl)-2-oxoethyl)piperidin-4-yl)-3-methyl-2-oxo-2,3-dihydro-1H-benzo[d]imidazol-1-yl)piperidine-2,6-dione (Example 115)

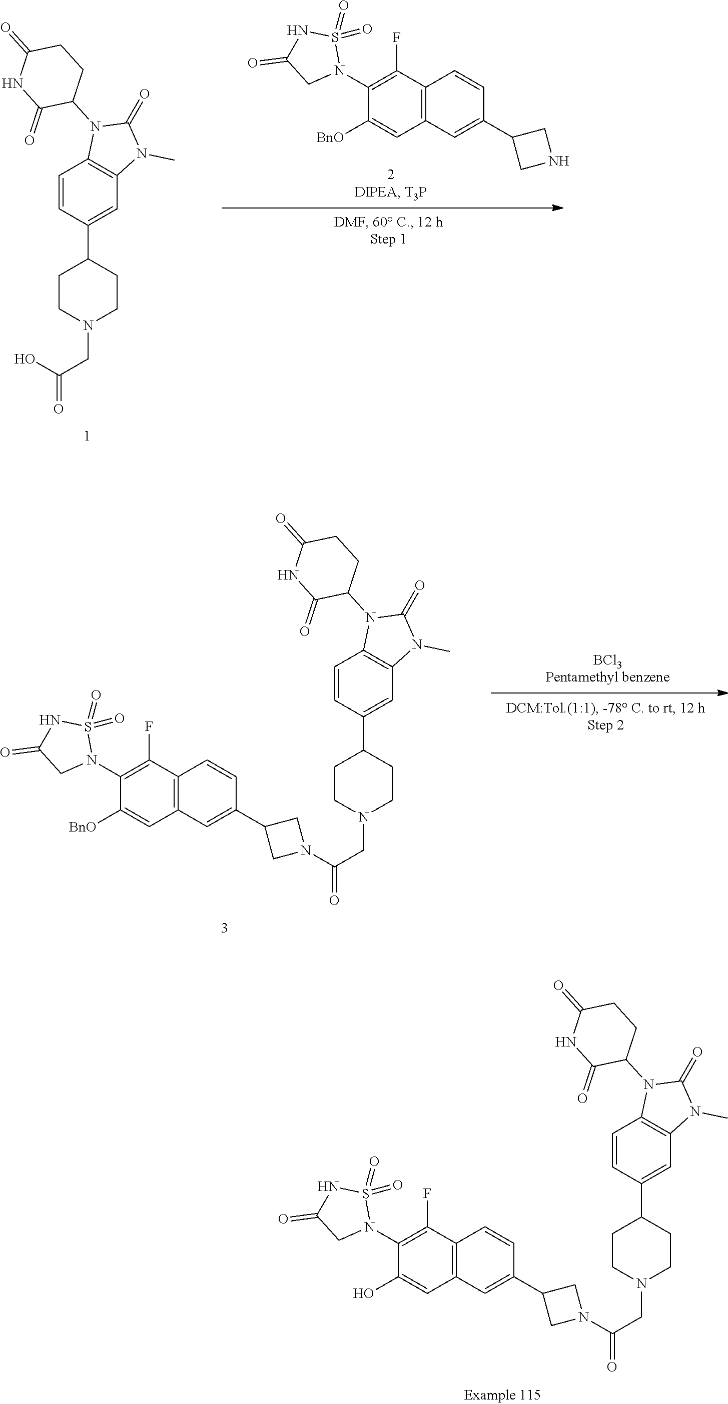 [SMILES: Cn1c(=O)n(C2CCC(=O)NC2=O)c2ccc(C3CCN(CC(=O)O)CC3)cc21 ; O=C1CN(c2c(OCc3ccccc3)cc3cc(C4CNC4)ccc3c2F)S(=O)(=O)N1 ; Cn1c(=O)n(C2CCC(=O)NC2=O)c2ccc(C3CCN(CC(=O)N4CC(c5ccc6c(F)c(N7CC(=O)NS7(=O)=O)c(OCc7ccccc7)cc6c5)C4)CC3)cc21 ; Cn1c(=O)n(C2CCC(=O)NC2=O)c2ccc(C3CCN(CC(=O)N4CC(c5ccc6c(F)c(N7CC(=O)NS7(=O)=O)c(O)cc6c5)C4)CC3)cc21]

### Step 1: 3-(5-(1-(2-(3-(7-(benzyloxy)-6-(1,1-dioxido-4-oxo-1,2,5-thiadiazolidin-2-yl)-5-fluoronaphthalen-2-yl)azetidin-1-yl)-2-oxoethyl)piperidin-4-yl)-3-methyl-2-oxo-2,3-dihydro-1H-benzo[d]imidazol-1-yl)piperidine-2,6-dione (3)

To a solution of 2-(4-(1-(2,6-dioxopiperidin-3-yl)-3-methyl-2-oxo-2,3-dihydro-1H-benzo[d]imidazol-5-yl)piperidin-1-yl)acetic acid (1, 125 mg, 286.11 μmol, HCl salt) and 5-(6-(azetidin-3-yl)-3-(benzyloxy)-1-fluoronaphthalen-2-yl)-1,2,5-thiadiazolidin-3-one 1,1-dioxide (2, 160 mg, 288.03 μmol, TFA salt) and DIPEA (184.89 mg, 1.43 mmol, 249.18 μL) in DMF (2 mL) was added T₃P (50% in ethyl acetate) (364.14 mg, 572.23 μmol). The mixture was stirred at 60° C. for 16 h. The mixture was poured into ethyl acetate (50) mL). The precipitate was filtered and dried under vacuum to afford 3-(5-(1-(2-(3-(7-(benzyloxy)-6-(1,1-dioxido-4-oxo-1,2,5-thiadiazolidin-2-yl)-5-fluoronaphthalen-2-yl)azetidin-1-yl)-2-oxoethyl)piperidin-4-yl)-3-methyl-2-oxo-2,3-dihydro-1H-benzo[d]imidazol-1-yl)piperidine-2,6-dione (3, 130 mg, 132.54 μmol, 46% yield). LCMS (ESI): m/z 824.3 [M+H]⁺

### Step 2: 3-(5-(1-(2-(3-(6-(1,1-dioxido-4-oxo-1,2,5-thiadiazolidin-2-yl)-5-fluoro-7-hydroxynaphthalen-2-yl)azetidin-1-yl)-2-oxoethyl)piperidin-4-yl)-3-methyl-2-oxo-2,3-dihydro-1H-benzo[d]imidazol-1-yl)piperidine-2,6-dione (Example 115)

To a solution of 3-(5-(1-(2-(3-(7-(benzyloxy)-6-(1,1-dioxido-4-oxo-1,2,5-thiadiazolidin-2-yl)-5-fluoronaphthalen-2-yl)azetidin-1-yl)-2-oxoethyl)piperidin-4-yl)-3-methyl-2-oxo-2,3-dihydro-1H-benzo[d]imidazol-1-yl)piperidine-2,6-dione (3, 120 mg, 123.80 and μmol) pentamethylbenzene (55.06 mg, 371.41 μmol) in DCM (1 mL) and toluene (1 mL) was added BCl₃ (1.0 M in DCM) 3.71 mL) at −78° C. under N₂. The mixture was stirred at RT for 12 h. The reaction mixture was quenched with DCM/MeOH=10/1 (0.5 mL) at (° C.) and concentrated under reduced pressure. The residue was purified by reversed phase flash chromatography (flow: 40 mL/min; gradient: from 5-25% MeCN in water (0.1% TFA) over 8 min; column: I.D. 21 mm×H195 mm. Welch Ultimate XB_C18 20-40 μm: 120 Å) to afford 3-(5-(1-(2-(3-(6-(1,1-dioxido-4-oxo-1,2,5-thiadiazolidin-2-yl)-5-fluoro-7-hydroxynaphthalen-2-yl)azetidin-1-yl)-2-oxoethyl)piperidin-4-yl)-3-methyl-2-oxo-2,3-dihydro-1H-benzo[d]imidazol-1-yl)piperidine-2,6-dione (Example 115, 35.1 mg, 40.57 μmol, 33% yield, TFA salt). LCMS (ESI): m/z 734.5 [M+H]^{+ 1}H NMR (400 MHZ, DMSO-d₆) δ 11.10 (s, 1H), 9.95 (s, 1H), 9.68 (s, 1H), 7.93 (d, J=8.6 HZ, 1H), 7.73 (s, 1H), 7.43 (dd, J=8.7, 1.6 Hz, 1H), 7.12-7.03 (m, 3H), 6.93 (d, J=8.5 Hz, 1H), 5.36 (dd, J=12.7, 5.4 Hz, 1H), 4.70-4.59 (m, 1H), 4.50-4.39 (m, 1H), 4.36-4.25 (m, 1H), 4.19-4.02 (m, 6H), 3.64-3.55 (m, 2H), 3.22-3.07 (m, 2H), 2.99-2.81 (m, 2H), 2.75-2.58 (m, 2H), 2.15-1.92 (m, 6H).

### N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)phenyl)propanamide (Example 116)

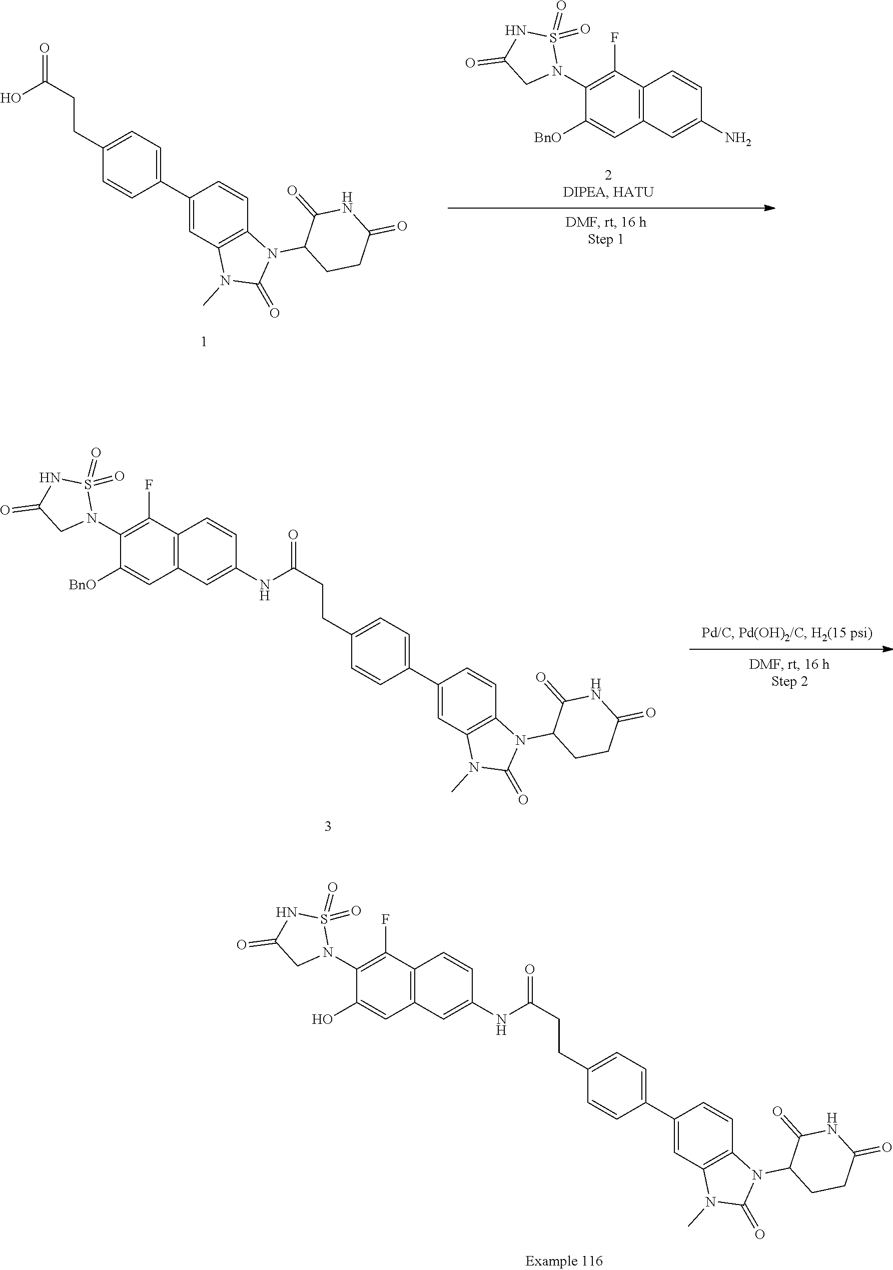 [SMILES: Cn1c(=O)n(C2CCC(=O)NC2=O)c2ccc(-c3ccc(CCC(=O)O)cc3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3ccc(CCC(=O)Nc4ccc5c(F)c(N6CC(=O)NS6(=O)=O)c(OCc6ccccc6)cc5c4)cc3)cc21 ; Cn1c(=O)n(C2CCC(=O)NC2=O)c2ccc(-c3ccc(CCC(=O)Nc4ccc5c(F)c(N6CC(=O)NS6(=O)=O)c(O)cc5c4)cc3)cc21]

### Step 1: N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)phenyl)propanamide (3)

To of 3-(4-(1-(2,6-dioxopiperidin-3-yl)-3-methyl-2-oxo-2,3-dihydro-1H-a solution benzo[d]imidazol-5-yl)phenyl)propanoic acid (1, 350 mg, 859.07 μmol, HCl salt) in DMF (3 mL) was added HATU (361.21 mg, 944.97 μmol) and DIPEA (555.14 mg, 4.30 mmol, 748.17 μL). After 20 min, 5-(6-amino-3-benzyloxy-1-fluoro-2-naphthyl)-1,1-dioxo-1,2,5-thiadiazolidin-3-one (2, 486.59 mg, 859.07 μmol, TFA salt) was added. After 16 h, the reaction mixture was filtered and concentrated under reduced pressure to afford N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)phenyl)propanamide (3, 1 g, 847.23 μmol). The material was used in the next step without purification. LCMS (ESI): m/z 791.3 [M+H]⁺

### Step 2: N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)phenyl)propanamide (Example 116)

To a solution of N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)phenyl)propanamide (3, 980 mg, 830.28 μmol) in DMF (5 mL) was added Pd/C (20 mg) under N₂ atmosphere. The suspension was degassed and purged with H₂. The mixture was stirred under H₂ (15 psi) at 20° C. for 16 h. The mixture was filtered and concentrated. The residue was purified by reversed phase flash chromatography (flow: 40 mL/min; gradient: from 0-28% MeCN in water (0.1% TFA) over 20 min; column: I.D. 31 mm×H140 mm, Welch Ultimate XB_C1820-40 μm: 120 Å) to afford N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)phenyl)propanamide (Example 116, 172 mg, 230.74 μmol, 28% yield) as an off-white solid. LCMS (ESI): m/z 701.2 [M+H]⁺

¹H NMR (400 MHz, DMSO-d₆) δ 11.11 (s, 1H), 10.21 (s, 1H), 8.19 (s, 1H), 7.85 (d, J=8.7 Hz, 1H), 7.62 (d, J=8.1 Hz, 2H), 7.48 (d, J=1.7 Hz, 1H), 7.46-7.40 (m, 1H), 7.39-7.29 (m, 3H), 7.17 (d, J=8.3 Hz, 1H), 6.97 (s, 1H), 5.39 (dd, J=12.9, 5.4 Hz, 1H), 4.47-4.37 (m, 2H), 3.40 (s, 3H), 3.03-2.96 (m, 2H), 2.94-2.85 (m, 1H), 2.79-2.64 (m, 4H), 2.10-1.96 (m, 1H).

### N-(6-(1,1-dioxido-4-oxo-1,2,5-thiadiazolidin-2-yl)-5-fluoro-7-hydroxynaphthalen-2-yl)-3-(4-(3-(2,6-dioxopiperidin-3-yl)-1-methyl-1H-indazol-6-yl)-3,3-difluoropiperidin-1-yl)cyclobutanecarboxamide (Example 117)

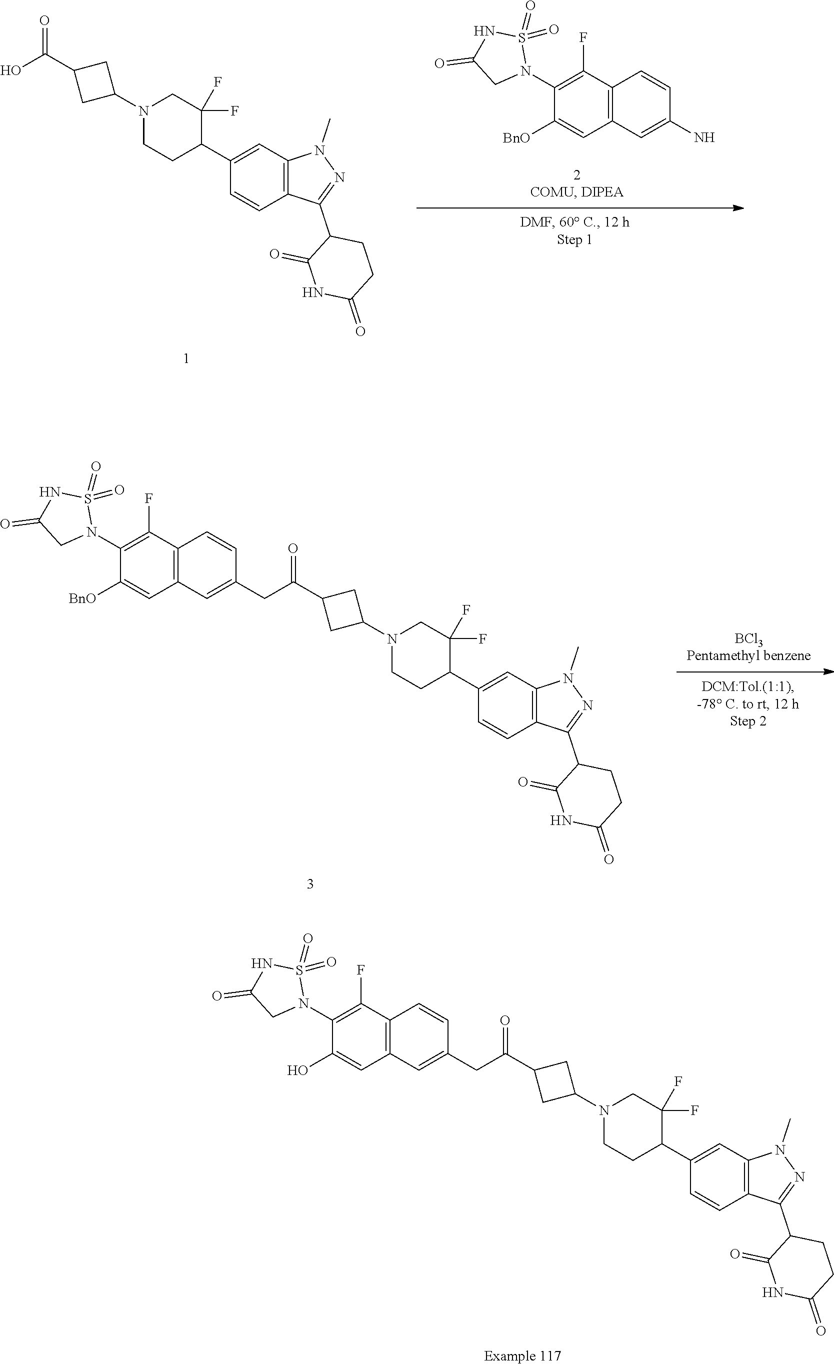 [SMILES: Cn1nc(C2CCC(=O)NC2=O)c2ccc(C3CCN(C4CC(C(=O)O)C4)CC3(F)F)cc21 ; Nc1ccc2c(F)c(N3CC(=O)NS3(=O)=O)c(OCc3ccccc3)cc2c1 ; Cn1nc(C2CCC(=O)NC2=O)c2ccc(C3CCN(C4CC(C(=O)Cc5ccc6c(F)c(N7CC(=O)NS7(=O)=O)c(OCc7ccccc7)cc6c5)C4)CC3(F)F)cc21 ; Cn1nc(C2CCC(=O)NC2=O)c2ccc(C3CCN(C4CC(C(=O)Cc5ccc6c(F)c(N7CC(=O)NS7(=O)=O)c(O)cc6c5)C4)CC3(F)F)cc21]

### Step 1: N-(7-(benzyloxy)-6-(1,1-dioxido-4-oxo-1,2,5-thiadiazolidin-2-yl)-5-fluoronaphthalen-2-yl)-3-(4-(3-(2,6-dioxopiperidin-3-yl)-1-methyl-1H-indazol-6-yl)-3,3-difluoropiperidin-1-yl)cyclobutanecarboxamide (3)

To a solution of 3-(4-(3-(2,6-dioxopiperidin-3-yl)-1-methyl-1H-indazol-6-yl)-3,3-difluoropiperidin-1-yl)cyclobutanecarboxylic acid (1, 150 mg, 281.22 μmol, HCl salt) in DMF (3 mL) were added 5-(6-amino-3-(benzyloxy)-1-fluoronaphthalen-2-yl)-1,2,5-thiadiazolidin-3-one 1,1-dioxide (2, 144.95 mg, 281.22 μmol, TFA salt), DIPEA (181.73 mg, 1.41 mmol, 244.92 μL) and COMU (180.65 mg, 421.83 μmol). The mixture was stirred at 60° C. for 12 h. The mixture was poured into ethyl acetate (40 mL) and the precipitate dried in vacuo to afford N-(7-(benzyloxy)-6-(1,1-dioxido-4-oxo-1,2,5-thiadiazolidin-2-yl)-5-fluoronaphthalen-2-yl)-3-(4-(3-(2,6-dioxopiperidin-3-yl)-1-methyl-1H-indazol-6-yl)-3,3-difluoropiperidin-1-yl)cyclobutanecarboxamide (3, 180 mg, 95.99 μmol, 34% yield, 45% purity) as a yellow solid. LCMS (ESI): m/z 844.3 [M+H]⁺

### Step 2: N-(6-(1,1-dioxido-4-oxo-1,2,5-thiadiazolidin-2-yl)-5-fluoro-7-hydroxynaphthalen-2-yl)-3-(4-(3-(2,6-dioxopiperidin-3-yl)-1-methyl-1H-indazol-6-yl)-3,3-difluoropiperidin-1-yl)cyclobutanecarboxamide (Example 117)

To a solution of N-(7-(benzyloxy)-6-(1,1-dioxido-4-oxo-1,2,5-thiadiazolidin-2-yl)-5-fluoronaphthalen-2-yl)-3-(4-(3-(2,6-dioxopiperidin-3-yl)-1-methyl-1H-indazol-6-yl)-3,3-difluoropiperidin-1-yl)cyclobutanecarboxamide (3, 180 mg, 95.99 μmol) and pentamethylbenzene (42.69 mg, 287.96 μmol) in toluene (4 mL) and DCM (4 mL) was added BCl₃ (1.0 M in DCM) (2.88 mL) at −78° C. under N₂. After 12 h. the mixture was concentrated and the residue was purified by reversed phase flash chromatography (flow: 40 mL/min; gradient: from 0-45% MeCN in water (0.1% TFA) over 8 min; column: I.D. 21 mm×H195 mm; Welch Ultimate XB_C18 20-40 μm: 120 Å) and prep-HPLC (flow: 25 mL/min. gradient: from 8-38% MeCN in (0.05% TFA) in water over 10 min; column: 3_Phenomenex Luna C18 75×30 mm×3 μm) to afford N-(6-(1,1-dioxido-4-oxo-1,2,5-thiadiazolidin-2-yl)-5-fluoro-7-hydroxynaphthalen-2-yl)-3-(4-(3-(2,6-dioxopiperidin-3-yl)-1-methyl-1H-indazol-6-yl)-3,3-difluoropiperidin-1-yl)cyclobutanecarboxamide (Example 117, 8.2 mg, 9.17 μmol, 10% yield, TFA salt) as a white solid. LCMS (ESI): m/z 754.4 [M+H]⁺

¹H NMR (400 MHZ, DMSO-d₆) δ 10.91 (s, 1H), 10.22 (d, J=12.4 Hz, 1H), 10.05 (s, 1H), 8.17 (d, J=16.7 Hz, 1H), 7.85 (d, J=9.0 Hz, 1H), 7.70 (d, J=8.4 Hz, 1H), 7.56-7.41 (m, 2H), 7.13-7.04 (m, 1H), 6.96 (s, 1H), 4.37 (dd, J=10.1, 5.1 Hz, 1H), 4.20 (s, 2H), 4.01 (s, 3H), 3.07-2.96 (m, 2H), 2.71-2.57 (m, 5H), 2.42-2.31 (m, 3H), 2.23-2.06 (m, 2H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-fluorophenyl)acetamide (Example 118)

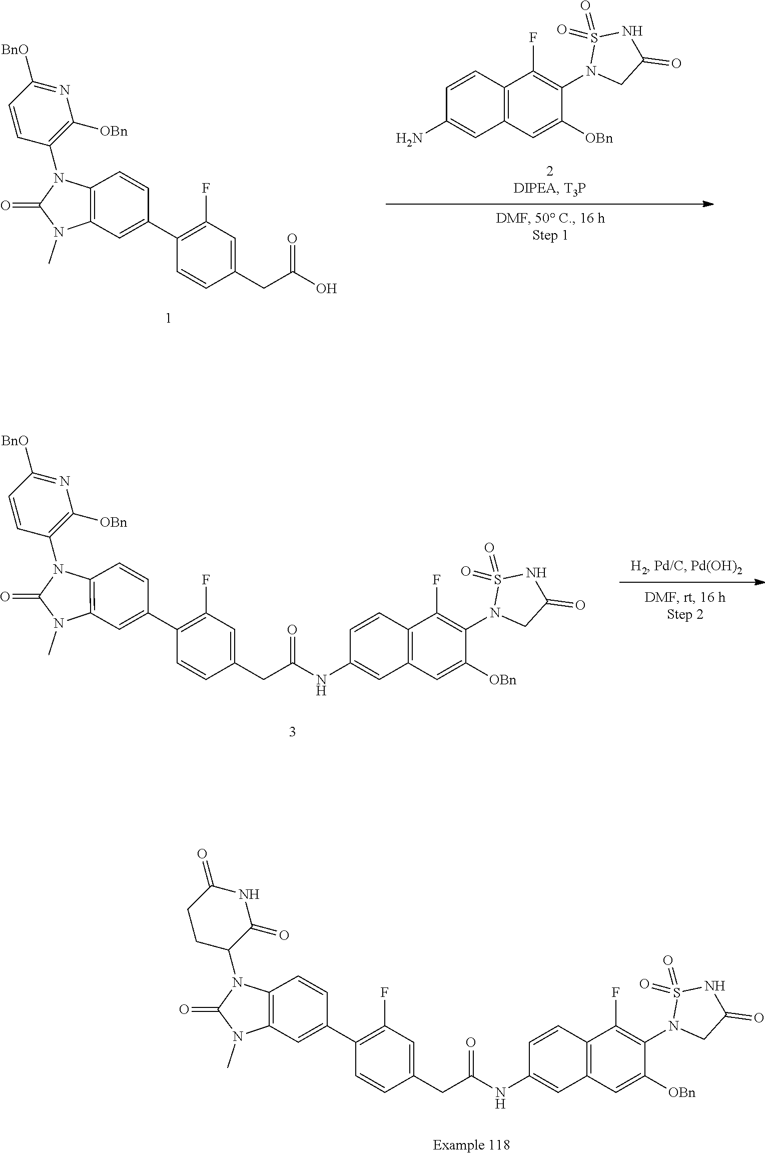 [SMILES: Cn1c(=O)n(-c2ccc(OCc3ccccc3)nc2OCc2ccccc2)c2ccc(-c3ccc(CC(=O)O)cc3F)cc21 ; Nc1ccc2c(F)c(N3CC(=O)NS3(=O)=O)c(OCc3ccccc3)cc2c1 ; Cn1c(=O)n(-c2ccc(OCc3ccccc3)nc2OCc2ccccc2)c2ccc(-c3ccc(CC(=O)Nc4ccc5c(F)c(N6CC(=O)NS6(=O)=O)c(OCc6ccccc6)cc5c4)cc3F)cc21 ; Cn1c(=O)n(C2CCC(=O)NC2=O)c2ccc(-c3ccc(CC(=O)Nc4ccc5c(F)c(N6CC(=O)NS6(=O)=O)c(OCc6ccccc6)cc5c4)cc3F)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-3-fluorophenyl)acetamide (3)

To a mixture of 2-(4-(1-(2,6-bis(benzyloxy)pyridin-3-yl)-3-methyl-2-oxo-2,3-dihydro-1H-benzo[d]imidazol-5-yl)-3-fluorophenyl)acetic acid (1, 140 mg, 237.44 μmol) and 5-(6-amino-3-(benzyloxy)-1-fluoronaphthalen-2-yl)-1,2,5-thiadiazolidin-3-one 1,1-dioxide (2, 95.31 mg, 237.44 μmol) in DMF (5 mL) were added DIPEA (460.32 mg, 3.56 mmol, 620.38 μL) and T₃P (50% in ethyl acetate) (755.50 mg, 1.19 mmol). The mixture was stirred at 50° C. for 16 h. The mixture was diluted with water (20 mL) and extracted with ethyl acetate (20 mL×2). The combined organic layers were washed with brine (20 mL×2), dried over Na₂SO₄, filtered and concentrated under reduced pressure. The residue was purified by column chromatography (SiO₂, DCM/MeOH=50/1 to 5/1) to afford N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-3-fluorophenyl)acetamide (3, 120 mg, 123.33 μmol, 52% yield). LCMS (ESI): m/z 973.3 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-fluorophenyl)acetamide (Example 118)

To a mixture of N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-3-fluorophenyl)acetamide (3, 90 mg, 92.50 μmol) in DMF (4 mL) were added Pd(OH)₂ (20 mg) and Pd/C (20 mg) under N₂ atmosphere. The mixture was stirred at 25° C. for 16 h under H₂ (15 psi). The mixture was filtered through Celite and washed with DMF (1 mL). The filtrate was purified by prep-HPLC (flow: 60 mL/min; gradient: from 9-39% MeCN in water (10 mM NH₄HCO₃) over 10 min; column: Waters Xbridge 150×25 mm×5 μm) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)-3-fluorophenyl)acetamide (Example 118, 39.25 mg, 54.59 μmol, 59% yield). LCMS (ESI): m/z 705.2 [M+H]⁺

¹H NMR (400 MHZ, DMSO-d₆) δ 10.42 (s, 1H), 8.16-8.12 (m, 1H), 7.84 (d, J=9.0 Hz, 1H), 7.52 (t, J=8.2 Hz, 1H), 7.45 (dd, J=9.0, 2.0 Hz, 1H), 7.37 (s, 1H), 7.35-7.27 (m, 3H), 7.22 (s, 2H), 6.94 (s, 1H), 5.41 (dd, J=12.9, 5.4 Hz, 1H), 4.06 (s, 2H), 3.78 (s, 2H), 3.38 (s, 3H), 2.98-2.85 (m, 1H), 2.83-2.71 (m, 2H), 2.14-1.99 (m, 1H).

### 3-[5-[1-[2-[4-[8-fluoro-6-hydroxy-7-(1,1,4-trioxo-1,2,5-thiadiazolidin-2-yl)-2-naphthyl]-3,6-dihydro-2H-pyridin-1-yl]-2-oxo-ethyl]-4-piperidyl]-3-methyl-2-oxo-benzimidazol-1-yl]piperidine-2,6-dione (Example 119)

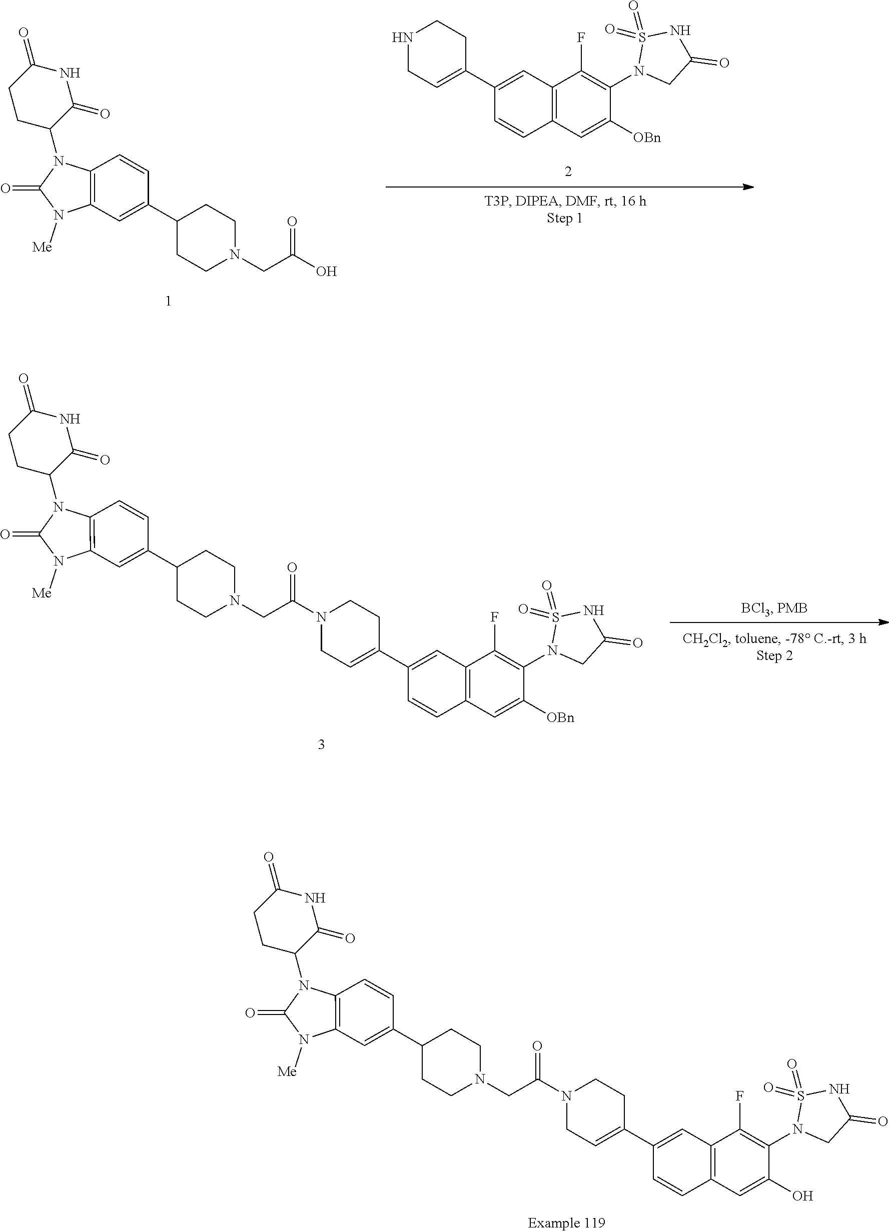 [SMILES: Cn1c(=O)n(C2CCC(=O)NC2=O)c2ccc(C3CCN(CC(=O)O)CC3)cc21 ; O=C1CN(c2c(OCc3ccccc3)cc3ccc(C4=CCNCC4)cc3c2F)S(=O)(=O)N1 ; Cn1c(=O)n(C2CCC(=O)NC2=O)c2ccc(C3CCN(CC(=O)N4CC=C(c5ccc6cc(OCc7ccccc7)c(N7CC(=O)NS7(=O)=O)c(F)c6c5)CC4)CC3)cc21 ; Cn1c(=O)n(C2CCC(=O)NC2=O)c2ccc(C3CCN(CC(=O)N4CC=C(c5ccc6cc(O)c(N7CC(=O)NS7(=O)=O)c(F)c6c5)CC4)CC3)cc21]

### Step 1: 3-[5-[1-[2-[4-[6-benzyloxy-8-fluoro-7-(1,1,4-trioxo-1,2,5-thiadiazolidin-2-yl)-2-naphthyl]-3,6-dihydro-2H-pyridin-1-yl]-2-oxo-ethyl]-4-piperidyl]-3-methyl-2-oxo-benzimidazol-1-yl]piperidine-2,6-dione (3)

Into a 50 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (1, 250 mg, 437.36 μmol, TFA salt) in dry DMF (4 mL) were added 5-[3-benzyloxy-1-fluoro-7-(1,2,3,6-tetrahydropyridin-4-yl)-2-naphthyl]-1,1-dioxo-1,2,5-thiadiazolidin-3-one (2, 405.00 mg, 437.36 μmol, TFA salt), 1-propanephosphonic anhydride (50% in EtOAc) (556.6 mg, 874.72 μmol) and DIPEA (226.10 mg, 1.75 mmol, 304.72 μL). After 16 h, the volatiles were removed under reduced pressure and ice-cold water (20 mL) added. The precipitate was filtered and dried under vacuum to afford 3-[5-[1-[2-[4-[6-benzyloxy-8-fluoro-7-(1,1,4-trioxo-1,2,5-thiadiazolidin-2-yl)-2-naphthyl]-3,6-dihydro-2H-pyridin-1-yl]-2-oxo-ethyl]-4-piperidyl]-3-methyl-2-oxo-benzimidazol-1-yl]piperidine-2,6-dione (3, 250 mg, 237.37 μmol, 54% yield) as an off-white solid. The material was used in the next step without purification. LCMS (ES+): m/z 850.3 [M+H]⁺

### Step 2: 3-[5-[1-[2-[4-[8-fluoro-6-hydroxy-7-(1,1,4-trioxo-1,2,5-thiadiazolidin-2-yl)-2-naphthyl]-3,6-dihydro-2H-pyridin-1-yl]-2-oxo-ethyl]-4-piperidyl]-3-methyl-2-oxo-benzimidazol-1-yl]piperidine-2,6-dione (Example 119)

Into a 50 mL single neck round bottom flask containing a well-stirred solution of 3-[5-[1-[2-[4-[6-benzyloxy-8-fluoro-7-(1,1,4-trioxo-1,2,5-thiadiazolidin-2-yl)-2-naphthyl]-3,6-dihydro-2H-pyridin-1-yl]-2-oxo-ethyl]-4-piperidyl]-3-methyl-2-oxo-benzimidazol-1-yl]piperidine-2,6-dione (3, 250) mg, 294.14 μmol) in dry toulene (5 mL) and dry DCM (5 mL) was added pentamethylbenzene (218.03 mg, 1.47 mmol, 237.76 μL) under nitrogen atmosphere. The reaction mixture was cooled to −78° C. and BCl₃ (1.0 M in DCM) (4.41 mL, 4.41 mmol) was added. The resulting mixture was stirred at ambient temperature for 3 h. The reaction mixture was cooled to −78° C. and quenched with 5% MeOH in DCM (6 mL). The reaction mixture was concentrated under reduced pressure and purified by reverse-phase preparative HPLC [Column: Aquagold C18 (19×150) mm, 5 micron; Mobile phase A: 0.1% TFA in water and Mobile phase B: MeCN] afforded 3-[5-[1-[2-[4-[8-fluoro-6-hydroxy-7-(1,1,4-trioxo-1,2,5-thiadiazolidin-2-yl)-2-naphthyl]-3,6-dihydro-2H-pyridin-1-yl]-2-oxo-ethyl]-4-piperidyl]-3-methyl-2-oxo-benzimidazol-1-yl]piperidine-2,6-dione (Example 119, 49 mg, 50.59 μmol, 17% yield, TFA salt) as an off-white solid. LCMS (ES+): m/z 758.2 [M−H]⁻

¹H NMR (400 MHZ, DMSO-d₆) δ 11.11 (s, 1H), 10.18 (s, 1H), 9.51 (s, 1H), 7.83 (d, J=12.8 Hz, 1H), 7.78-7.66 (m, 2H), 7.07 (d, J=5.4 Hz, 3H), 6.94 (d, J=8.2 Hz, 1H), 6.46-6.37 (m, 1H), 5.36 (dd, J=12.7, 5.4 Hz, 1H), 4.46 (d, J=4.4 Hz, 1H), 4.40 (s, 0H), 4.25 (s, 1H), 4.23 (s, 2H), 4.16 (s, 1H), 3.85-3.81 (m, 2H), 3.35 (s, 3H), 3.22-3.08 (m, 2H), 2.98-2.82 (m, 2H), 2.81-2.71 (m, 2H), 2.70-2.58 (m, 2H), 2.16-2.07 (m, 2H), 2.06-1.96 (m, 3H).

### 3-[5-[1-[2-[4-[8-fluoro-6-hydroxy-7-(1,1,4-trioxo-1,2,5-thiadiazolidin-2-yl)-2-naphthyl]-1-piperidyl]-2-oxo-ethyl]-4-piperidyl]-3-methyl-2-oxo-benzimidazol-1-yl]piperidine-2,6-dione (Example 120)

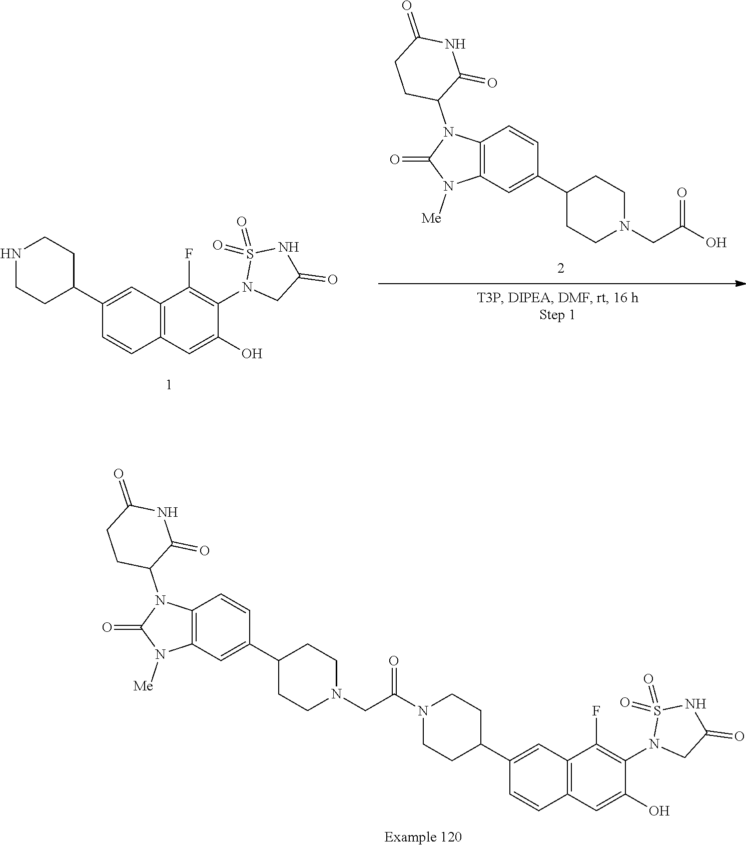 [SMILES: O=C1CN(c2c(O)cc3ccc(C4CCNCC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)N4CCC(c5ccc6cc(O)c(N7CC(=O)NS7(=O)=O)c(F)c6c5)CC4)CC3)cc21]

### Step 1: 3-[5-[1-[2-[4-[8-fluoro-6-hydroxy-7-(1,1,4-trioxo-1,2,5-thiadiazolidin-2-yl)-2-naphthyl]-1-piperidyl]-2-oxo-ethyl]-4-piperidyl]-3-methyl-2-oxo-benzimidazol-1-yl]piperidine-2,6-dione (Example 120)

Into a 25 mL single neck, round bottom flask containing a well-stirred solution of 2-[4-[1-(2,6-dioxo-3-piperidyl)-3-methyl-2-oxo-benzimidazol-5-yl]-1-piperidyl]acetic acid (1, 200 mg, 388.76 μmol, TFA salt) and 5-[1-fluoro-3-hydroxy-7-(4-piperidyl)-2-naphthyl]-1,1-dioxo-1,2,5-thiadiazolidin-3-one (2, 211.01 mg, 427.64 μmol, TFA salt) in dry DMF (3 mL) were added DIPEA (200.98 mg, 1.56 mmol, 270.86 μL) and 1-propanephosphonic anhydride (50 Wt % in EtOAc) (494.7 mg, 777.53 μmol). After 16 h the volatiles were removed under reduced pressure and the residue was purified by reverse-phase preparative HPLC [Zorbax C18 (250×21.2), 7 micron; Mobile phase A: 10 mM NH₄OAc in water and Mobile phase B: CH₃CN] to afford 3-[5-[1-[2-[4-[8-fluoro-6-hydroxy-7-(1,1,4-trioxo-1,2,5-thiadiazolidin-2-yl)-2-naphthyl]-1-piperidyl]-2-oxo-ethyl]-4-piperidyl]-3-methyl-2-oxo-benzimidazol-1-yl]piperidine-2,6-dione (Example 120, 32 mg, 41.54 μmol, 11% yield) as an off-white solid. LCMS (ES+): m/z 762.2 [M+H]⁺

¹H NMR (400 MHZ, DMSO-d₆) δ 11.11 (s, 1H), 9.72 (s, 1H), 9.46 (s, 1H), 7.74-7.64 (m, 2H), 7.41 (dd, J=8.6, 1.7 Hz, 1H), 7.11-7.02 (m, 3H), 6.94 (d, J=8.3 Hz, 1H), 5.36 (dd, J=12.8, 5.4 Hz, 1H), 4.57 (d, J=12.7 Hz, 1H), 4.51-4.29 (m, 2H), 4.08 (s, 2H), 3.88-3.74 (m, 1H), 3.66-3.52 (m, 1H), 3.28-3.06 (m, 3H), 3.06-2.96 (m, 1H), 2.95-2.78 (m, 3H), 2.76-2.61 (m, 3H), 2.16-1.91 (m, 7H), 1.84-1.68 (m, 1H), 1.65-1.49 (m, 1H).

### 3-[5-[1-[2-[4-[8-fluoro-6-hydroxy-7-(1,1,4-trioxo-1,2,5-thiadiazolidin-2-yl)-2-naphthyl]-3,6-dihydro-2H-pyridin-1-yl]ethyl]-4-piperidyl]-3-methyl-2-oxo-benzimidazol-1-yl]piperidine-2,6-dione (Example 121)

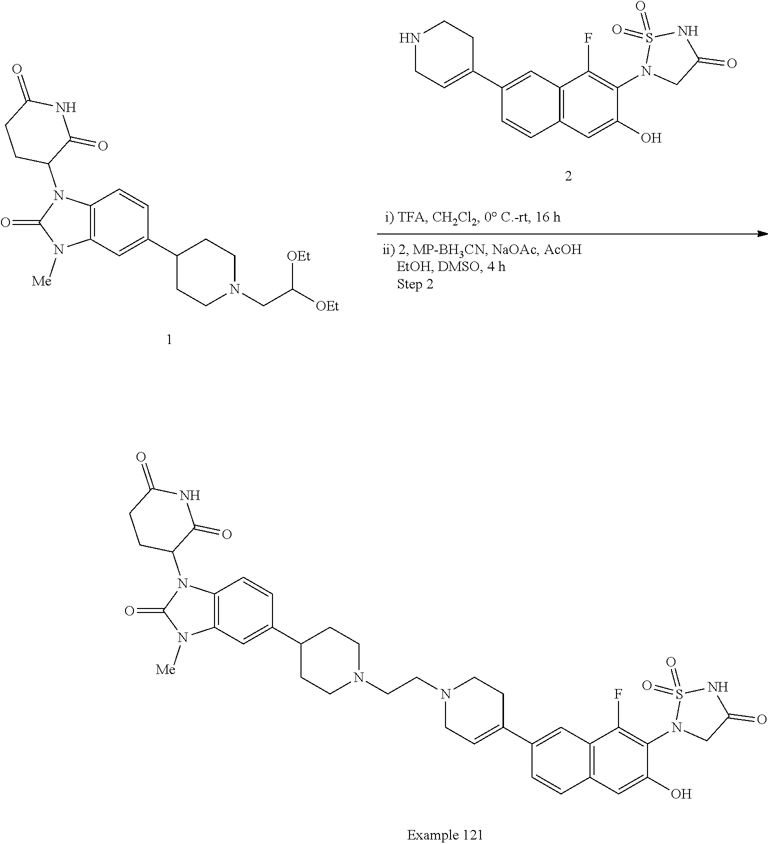 [SMILES: CCOC(CN1CCC(c2ccc3c(c2)n(C)c(=O)n3C2CCC(=O)NC2=O)CC1)OCC ; O=C1CN(c2c(O)cc3ccc(C4=CCNCC4)cc3c2F)S(=O)(=O)N1 ; Cn1c(=O)n(C2CCC(=O)NC2=O)c2ccc(C3CCN(CCN4CC=C(c5ccc6cc(O)c(N7CC(=O)NS7(=O)=O)c(F)c6c5)CC4)CC3)cc21]

### Step 1: 3-[5-[1-[2-[4-[8-fluoro-6-hydroxy-7-(1,1,4-trioxo-1,2,5-thiadiazolidin-2-yl)-2-naphthyl]-3,6-dihydro-2H-pyridin-1-yl]ethyl]-4-piperidyl]-3-methyl-2-oxo-benzimidazol-1-yl]piperidine-2,6-dione (Example 121)

Into a 25 mL single neck, round bottom flask containing a well-stirred solution of 3-[5-[1-(2,2-diethoxyethyl)-4-piperidyl]-3-methyl-2-oxo-benzimidazol-1-yl]piperidine-2,6-dione (1, 160 mg, 289.61 μmol) in DCM (3 mL) was added TFA (2.37 g, 20.77 mmol, 1.60 mL). After 16 h the reaction mixture was concentrated under reduced pressure and triturated with diethyl ether (2×5 mL) to afford the crude aldehyde (160 mg, not shown). To this aldehyde (160 mg) and 5-[1-fluoro-3-hydroxy-7-(1,2,3,6-tetrahydropyridin-4-yl)-2-naphthyl]-1,1-dioxo-1,2,5-thiadiazolidin-3-one (2, 268.08 mg, 376.49 μmol) in DMSO (1 mL) and ethanol (3 mL) were added NaOAc (59.39 mg, 724.02 μmol), acetic acid (173.92 mg, 2.90 mmol, 165.63 μL) and MP-cyanoborohydride (2.0 mmol/g, 300 mg, 0.6 mmol). The suspension was stirred at RT for 4 h after which it was filtered and washed with ethanol (10 mL). The filtrate was concentrated under reduced pressure and purified by reverse-phase preparative HPLC [Column: XSelect-C18 (150×19) 5 μm; Mobile phase A: 0.1% TFA in water and Mobile Phase B: CH₃CN] to afford 3-[5-[1-[2-[4-[8-fluoro-6-hydroxy-7-(1,1,4-trioxo-1,2,5-thiadiazolidin-2-yl)-2-naphthyl]-3,6-dihydro-2H-pyridin-1-yl]ethyl]-4-piperidyl]-3-methyl-2-oxo-benzimidazol-1-yl]piperidine-2,6-dione (Example 121, 54 mg, 57.72 μmol, 20% yield, TFA salt) as an off-white solid. LCMS (ES+): m/z 746.2 [M+H]^{+ 1}H NMR (400 MHZ, DMSO-d₆) δ 11.11 (s, 1H), 10.06 (s, 2H), 7.87 (s, 1H), 7.80-7.70 (m, 2H), 7.12-7.04 (m, 3H), 6.94 (d, J=8.2 Hz, 1H), 6.43 (s, 1H), 5.36 (dd, J=12.7, 5.4 Hz, 1H), 4.15 (s, 2H), 3.34 (s, 3H), 3.17-3.03 (m, 2H), 2.97-2.82 (m, 4H), 2.77-2.57 (m, 2H), 2.12-1.88 (m, 6H). Note: additional peaks under solvent signal

### 3-[5-[1-[2-[4-[5-fluoro-7-hydroxy-6-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (Example 122)

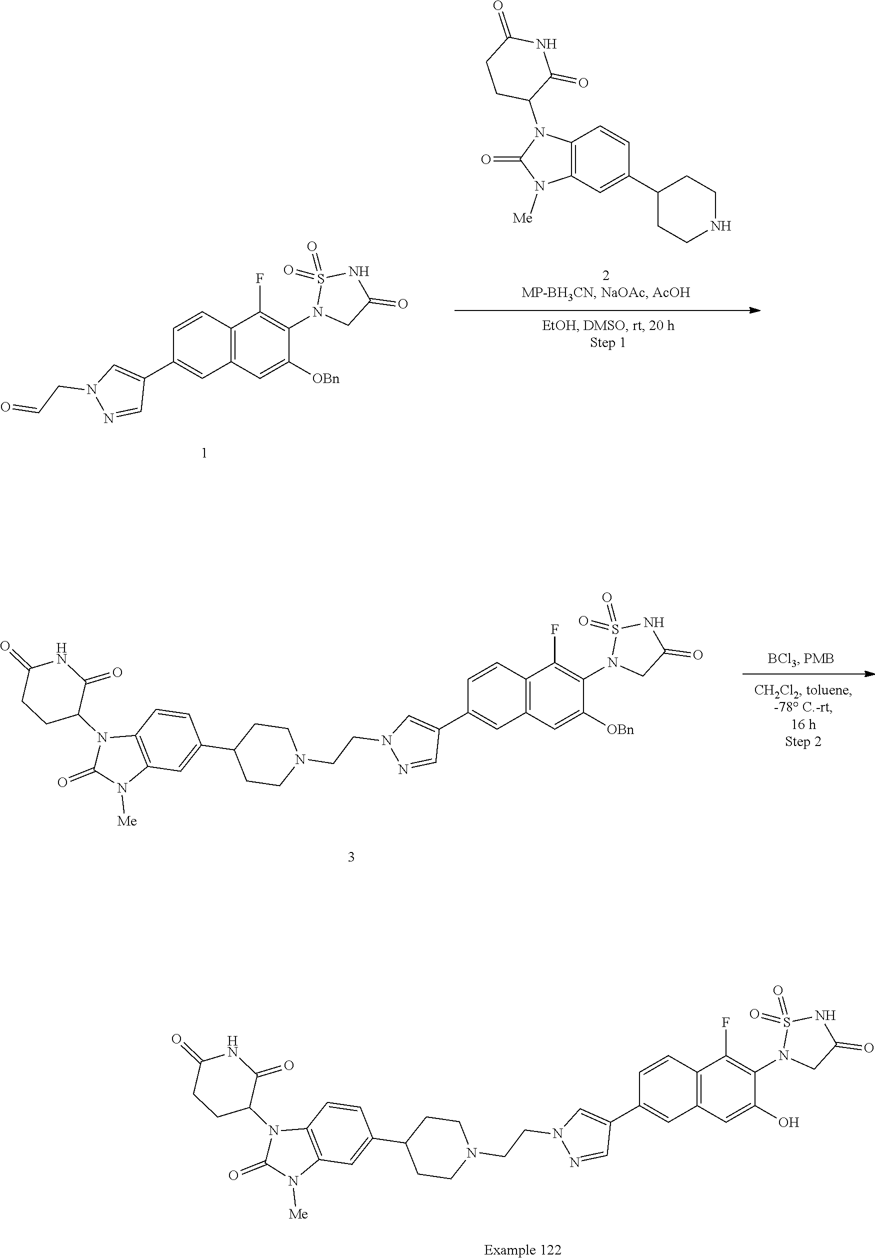 [SMILES: Cn1c(=O)n(C2CCC(=O)NC2=O)c2ccc(C3CCNCC3)cc21 ; O=CCn1cc(-c2ccc3c(F)c(N4CC(=O)NS4(=O)=O)c(OCc4ccccc4)cc3c2)cn1 ; Cn1c(=O)n(C2CCC(=O)NC2=O)c2ccc(C3CCN(CCn4cc(-c5ccc6c(F)c(N7CC(=O)NS7(=O)=O)c(OCc7ccccc7)cc6c5)cn4)CC3)cc21 ; Cn1c(=O)n(C2CCC(=O)NC2=O)c2ccc(C3CCN(CCn4cc(-c5ccc6c(F)c(N7CC(=O)NS7(=O)=O)c(O)cc6c5)cn4)CC3)cc21]

### Step 1: 3-[5-[1-[2-[4-[7-benzyloxy-5-fluoro-6-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (3)

Into a 10 mL single neck, round bottom flask containing a well-stirred suspension of 2-[4-[7-benzyloxy-5-fluoro-6-(1,1,4-trioxo-1,2,5-thiadiazolidin-2-yl)-2-naphthyl]pyrazol-1-yl]acetaldehyde (1, 250 mg, 230.07 μmol, TFA salt) and 3-[3-methyl-2-oxo-5-(4-piperidyl)benzimidazol-1-yl]piperidine-2,6-dione (2, 87.16 mg, 230.07 μmol, HCl salt) in EtOH (4 mL) and DCE (4 mL) were added NaOAc (94.36 mg, 1.15 mmol) and acetic acid (138.16 mg, 2.30 mmol, 131.58 μL). After 10 min, MP-cyanoborohydride (2.0 mmol/g, 200 mg, 0.4 mmol) was added. After 16 h, the reaction was filtered, concentrated under reduced pressure and purified by reverse-phase column chromatography [Mobile Phase A: 0.1% TFA in water and Mobile Phase B: CH₃CN] to afford 3-[5-[1-[2-[4-[7-benzyloxy-5-fluoro-6-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (3, 350 mg, 220.88 μmol, 96% yield, 59% purity, TFA salt). LCMS (ES−): m/z 819.0 [M−H]⁻

### Step 2: 3-[5-[1-[2-[4-[5-fluoro-7-hydroxy-6-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (Example 122)

Into a 25 mL round bottom flask containing a well-stirred solution of 3-[5-[1-[2-[4-[7-benzyloxy-5-fluoro-6-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (3, 260 mg, 186.87 μmol) in a mixture of dry toulene (5 mL) and DCM (5 mL) was added pentamethylbenzene (166.22 mg, 1.12 mmol) and the reaction mixture was cooled to −78° C. Then BCl₃ (1.0 M solution in DCM) (12.0) mmol, 12 mL) was added dropwise over a period of 2 min. Subsequently, the reaction mixture was brought to RT and stirred for 20 h. The reaction mixture was cooled to −78° C. and quenched slowly with 10% DCM in MeOH (10 mL). The solvent was removed under reduced pressure and the residue purified by reverse-phase preparative HPLC [Column: X-Select-C18 (150×19) 5 μm: Mobile phase A: 0.1% TFA in water and Mobile phase B: CH₃CN] to afford 3-[5-[1-[2-[4-[5-fluoro-7-hydroxy-6-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (Example 122, 45.4 mg, 53.42 μmol, 29% yield, TFA salt) as a white solid. LCMS (ES+): m/z 731.3 [M+H]⁺

¹H NMR (400 MHZ, DMSO-d₆) δ 11.10 (s, 1H), 9.84 (s, 1H), 9.27 (s, 1H), 8.46 (s, 1H), 8.20 (s, 1H), 7.97 (s, 1H), 7.89 (d, J=8.7 Hz, 1H), 7.63 (dd, J=8.7, 1.6 Hz, 1H), 7.10-7.02 (m, 3H), 6.91 (d, J=8.1 Hz, 1H), 5.35 (dd, J=12.8, 5.4 Hz, 1H), 4.72-4.56 (m, 2H), 4.09 (s, 2H), 3.76-3.58 (m, 4H), 3.22-3.07 (m, 1H), 2.97-2.81 (m, 2H), 2.76-2.69 (m, 1H), 2.65-2.57 (m, 3H), 2.10-1.83 (m, 5H).

### 3-[5-[1-[2-[4-[8-fluoro-6-hydroxy-7-(1,1,4-trioxo-1,2,5-thiadiazolidin-2-yl)-2-naphthyl]-1-piperidyl]ethyl]-4-piperidyl]-3-methyl-2-oxo-benzimidazol-1-yl]piperidine-2,6-dione (Example 123)

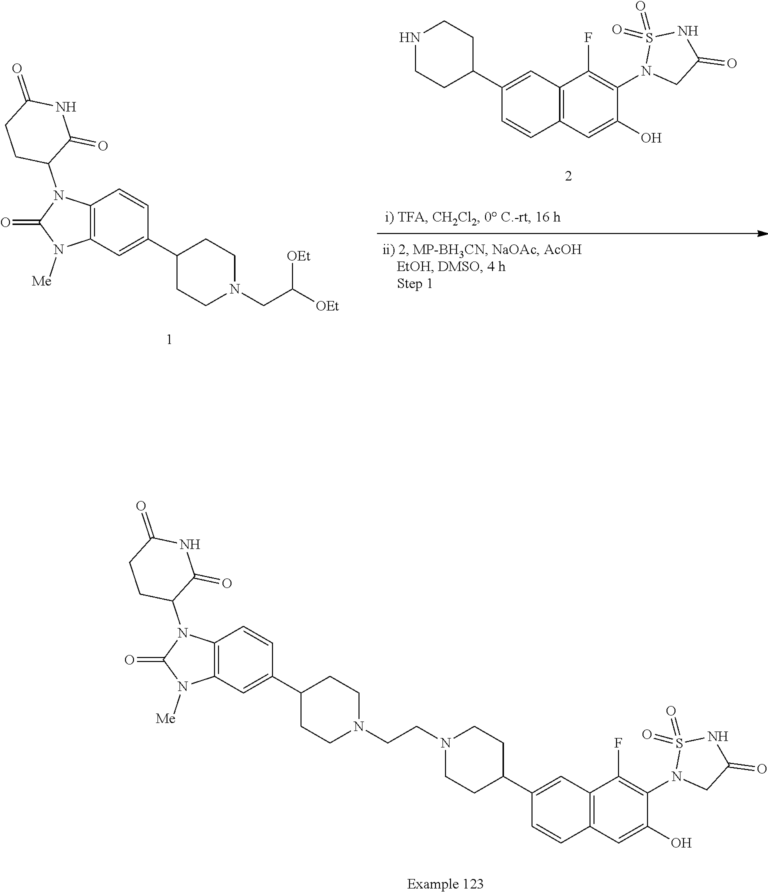 [SMILES: CCOC(CN1CCC(c2ccc3c(c2)n(C)c(=O)n3C2CCC(=O)NC2=O)CC1)OCC ; O=C1CN(c2c(O)cc3ccc(C4CCNCC4)cc3c2F)S(=O)(=O)N1 ; Cn1c(=O)n(C2CCC(=O)NC2=O)c2ccc(C3CCN(CCN4CCC(c5ccc6cc(O)c(N7CC(=O)NS7(=O)=O)c(F)c6c5)CC4)CC3)cc21]

### Step 1: 3-[5-[1-[2-[4-[8-fluoro-6-hydroxy-7-(1,1,4-trioxo-1,2,5-thiadiazolidin-2-yl)-2-naphthyl]-1-piperidyl]ethyl]-4-piperidyl]-3-methyl-2-oxo-benzimidazol-1-yl]piperidine-2,6-dione (Example 123)

Into a 25 mL single neck, round bottom flask containing a well-stirred solution of 3-[5-[1-(2,2-diethoxyethyl)-4-piperidyl]-3-methyl-2-oxo-benzimidazol-1-yl]piperidine-2,6-dione (1, 160 mg, 231.94 μmol, TFA salt) in DCM (3 mL) was added TFA (2.37 g, 20.77 mmol, 1.60 mL). After 16 h, the reaction mixture was concentrated under reduced pressure and triturated with diethyl ether (2×5 mL) to afford the crude aldehyde (160 mg, not shown). To this aldehyde (160 mg) and 5-[1-fluoro-3-hydroxy-7-(4-piperidyl)-2-naphthyl]-1,1-dioxo-1,2,5-thiadiazolidin-3-one (2, 228.89 mg, 347.90 μmol, TFA salt) in DMSO (1 mL) and ethanol (5 mL) were added NaOAc (47.57 mg, 579.84 μmol), acetic acid (139.28 mg, 2.32 mmol, 132.65 μL), MP-cyanoborohydride (2.0) mmol/g, 500 mg, 1.0 mmol). The resulting suspension was stirred at RT for 4 h after which it was filtered and washed with ethanol (10 mL). The filtrate was concentrated under reduced pressure and the residue was purified by reverse-phase preparative HPLC [Column: XSelect-C18 (150×19) 5 μm; Mobile phase A: 0.1% TFA in water and Mobile Phase B: CH₃CN] to afford 3-[5-[1-[2-[4-[8-fluoro-6-hydroxy-7-(1,1,4-trioxo-1,2,5-thiadiazolidin-2-yl)-2-naphthyl]-1-piperidyl]ethyl]-4-piperidyl]-3-methyl-2-oxo-benzimidazol-1-yl]piperidine-2,6-dione (Example 123, 51 mg, 58.36 μmol, 25% yield, TFA salt) as an off-white solid. LCMS (ES+): m/z 748.3 [M+H]⁺

¹H NMR (400 MHZ, DMSO-d₆) δ 11.11 (s, 1H), 7.84-7.66 (m, 2H), 7.43 (d, J=8.3 Hz, 1H), 7.18-7.04 (m, 3H), 6.96 (d, J=8.2 Hz, 1H), 5.37 (dd, J=12.7, 5.4 Hz, 1H), 4.16 (s, 2H), 3.35 (s, 3H), 3.21-2.96 (m, 5H), 2.96-2.85 (m, 2H), 2.78-2.58 (m, 3H), 2.22-1.88 (m, 9H). Note: additional peaks under solvent signal

### 3-[5-[4-[2-[4-[8-fluoro-6-hydroxy-7-(1,1,4-trioxo-1,2,5-thiadiazolidin-2-yl)-2-naphthyl]-1-piperidyl]-2-oxo-ethyl]-1-piperidyl]-3-methyl-2-oxo-benzimidazol-1-yl]piperidine-2,6-dione (Example 124)

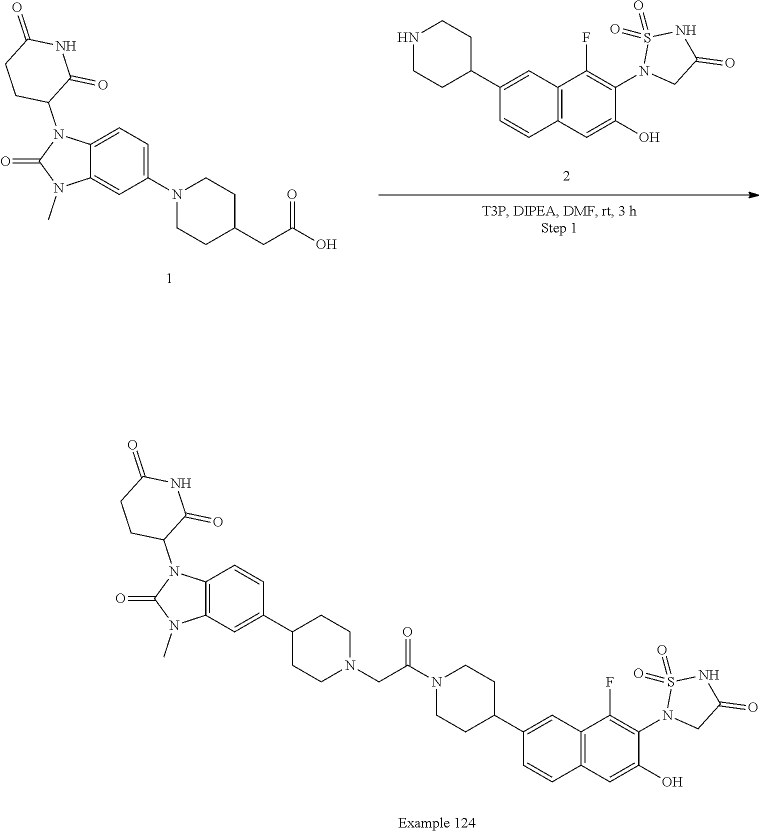 [SMILES: Cn1c(=O)n(C2CCC(=O)NC2=O)c2ccc(N3CCC(CC(=O)O)CC3)cc21 ; O=C1CN(c2c(O)cc3ccc(C4CCNCC4)cc3c2F)S(=O)(=O)N1 ; Cn1c(=O)n(C2CCC(=O)NC2=O)c2ccc(C3CCN(CC(=O)N4CCC(c5ccc6cc(O)c(N7CC(=O)NS7(=O)=O)c(F)c6c5)CC4)CC3)cc21]

### Step 1: 3-[5-[4-[2-[4-[8-fluoro-6-hydroxy-7-(1,1,4-trioxo-1,2,5-thiadiazolidin-2-yl)-2-naphthyl]-1-piperidyl]-2-oxo-ethyl]-1-piperidyl]-3-methyl-2-oxo-benzimidazol-1-yl]piperidine-2,6-dione (Example 124)

Into a 50 mL single neck round bottom flask containing a well-stirred solution of 2-[1-[1-(2,6-dioxo-3-piperidyl)-3-methyl-2-oxo-benzimidazol-5-yl]-4-piperidyl]acetic acid (1, 200 mg, 450.12 μmol) in dry DMF (4 mL) were added 5-[1-fluoro-3-hydroxy-7-(4-piperidyl)-2-naphthyl]-1,1-dioxo-1,2,5-thiadiazolidin-3-one (2, 222.10 mg, 450.12 μmol, TFA salt), 1-propanephosphonic anhydride (50 Wt % in EtOAc) (429.6 mg, 675.18 μmol) and DIPEA (174.52 mg, 1.35 mmol, 235.21 μL). After 3 h, the reaction mixture was concentrated under reduced pressure and the residue was purified by preparative HPLC [Column: XSELECT C18 (19×150) mm, 5 micron; Mobile phase A: 0.1% TFA in water and Mobile phase B: CH₃CN] to obtain 3-[5-[4-[2-[4-[8-fluoro-6-hydroxy-7-(1,1,4-trioxo-1,2,5-thiadiazolidin-2-yl)-2-naphthyl]-1-piperidyl]-2-oxo-ethyl]-1-piperidyl]-3-methyl-2-oxo-benzimidazol-1-yl]piperidine-2,6-dione (Example 124, 39.15 mg, 44.07 μmol, 10% yield, TFA salt) as a red solid. LCMS (ES+): m/z 762.2 [M+H]⁺

¹H NMR (400 MHZ, DMSO-d₆) δ 11.14 (s, 1H), 10.12 (s, 1H), 7.76-7.66 (m, 2H), 7.54 (s, 1H), 7.44 (dd, J=8.5, 1.8 Hz, 1H), 7.24 (s, 2H), 7.05 (s, 1H), 5.41 (dd, J=12.7, 5.4 Hz, 1H), 4.62 (d, J=12.6 Hz, 1H), 4.28 (s, 2H), 4.09 (d, J=13.0 Hz, 1H), 3.66-3.58 (m, 2H), 3.37 (s, 3H), 3.22-3.11 (m, 1H), 3.01-2.85 (m, 2H), 2.78-2.59 (m, 3H), 2.22-1.99 (m, 4H), 1.90 (t, J=13.0 Hz, 2H), 1.76-1.45 (m, 4H).

### 3-[5-[1-[[1-[8-fluoro-6-hydroxy-7-(1,1,4-trioxo-1,2,5-thiadiazolidin-2-yl)-2-naphthyl]pyrazol-4-yl]methyl]-4-piperidyl]-3-methyl-2-oxo-benzimidazol-1-yl]piperidine-2,6-dione (Example 125)

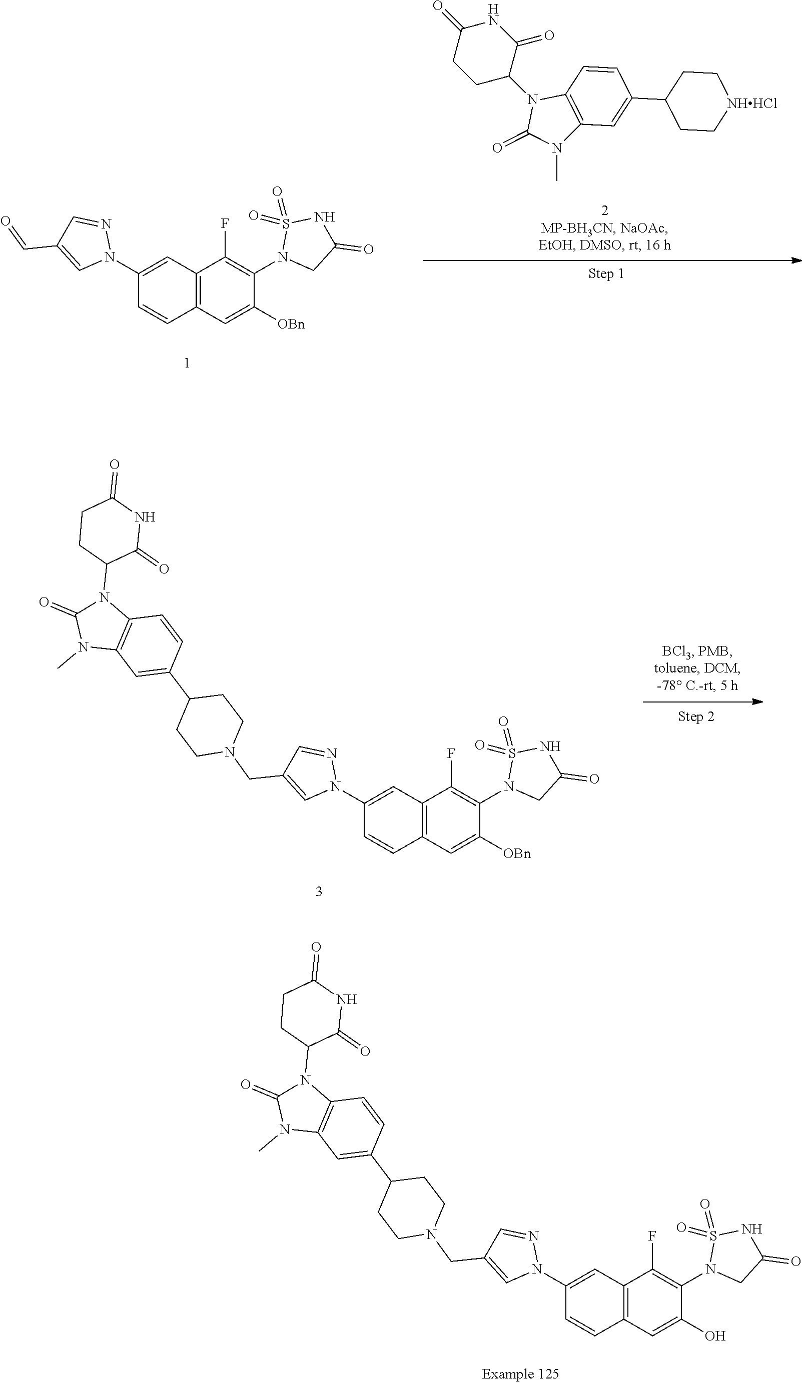

### Step 1: 3-[5-[1-[[1-[6-benzyloxy-8-fluoro-7-(1,1,4-trioxo-1,2,5-thiadiazolidin-2-yl)-2-naphthyl]pyrazol-4-yl]methyl]-4-piperidyl]-3-methyl-2-oxo-benzimidazol-1-yl]piperidine-2,6-dione (3)

Into a 50 mL single neck round bottom flask containing a well-stirred solution of 1-[6-benzyloxy-8-fluoro-7-(1,1,4-trioxo-1,2,5-thiadiazolidin-2-yl)-2-naphthyl]pyrazole-4-carbaldehyde (1, 300 mg, 605.66 μmol) and 3-[3-methyl-2-oxo-5-(4-piperidyl)benzimidazol-1-yl]piperidine-2,6-dione (2, 256.54 mg, 749.27 μmol, HCl salt) in DMSO (5 mL) and ethanol (5 mL) were added sodium acetate (153.66 mg, 1.87 mmol, 100.43 μL) and acetic acid (749.89 mg, 12.49 mmol, 714.19 μL). After 10 min, MP-cyanoborohydride (2 mmol per 1 g) (81.76 mg, 1.82 mmol) was added. After 16 h, the reaction mixture was filtered through Celite and washed with ethanol (20 mL). The filtrate was concentrated under reduced pressure and the residue was purified by reverse phase column chromatography [Purification method: Siliasep premium C18, 25 μm, 120 g column; Mobile phase A: 0.1% TFA in water; Mobile phase B: Acetonitrile] to afford 3-[5-[1-[[1-[6-benzyloxy-8-fluoro-7-(1,1,4-trioxo-1,2,5-thiadiazolidin-2-yl)-2-naphthyl]pyrazol-4-yl]methyl]-4-piperidyl]-3-methyl-2-oxo-benzimidazol-1-yl]piperidine-2,6-dione (3, 150 mg, 162.15 μmol, 26% yield) as a pale yellow solid. LCMS (ES+): m/z 807.2 [M+H]⁺

### Step 2: 3-[5-[1-[[1-[8-fluoro-6-hydroxy-7-(1,1,4-trioxo-1,2,5-thiadiazolidin-2-yl)-2-naphthyl]pyrazol-4-yl]methyl]-4-piperidyl]-3-methyl-2-oxo-benzimidazol-1-yl]piperidine-2,6-dione (Example 125)

Into a 50 mL single neck round bottom flask containing a well-stirred solution of 3-[5-[1-[[1-[6-benzyloxy-8-fluoro-7-(1,1,4-trioxo-1,2,5-thiadiazolidin-2-yl)-2-naphthyl]pyrazol-4-yl]methyl]-4-piperidyl]-3-methyl-2-oxo-benzimidazol-1-yl]piperidine-2,6-dione (3, 230 mg, 285.06 μmol) in toluene (3 mL) and DCM (3 mL) was added pentamethylbenzene (211.29 mg, 1.43 mmol, 230.41 μL). Boron trichloride (1.0 M in DCM) (5.7 mL, 5.70 mmol) was added at −75° C. The reaction mixture was stirred at RT for 5 h. The reaction was quenched with 5% MeOH in DCM (1.5 mL) at −75° C. The reaction mixture was concentrated under reduced pressure and the residue triturated with diethyl ether (10 mL). The material was purified by reverse phase prep HPLC [Purification method: Column: Zorbax C18 (250×21.2), 7 micron; Mobile phase A: 0.1% TFA in water; Mobile phase B: MeCN] to afford 3-[5-[1-[[1-[8-fluoro-6-hydroxy-7-(1,1,4-trioxo-1,2,5-thiadiazolidin-2-yl)-2-naphthyl]pyrazol-4-yl]methyl]-4-piperidyl]-3-methyl-2-oxo-benzimidazol-1-yl]piperidine-2,6-dione (Example 125, 35 mg, 41.82 μmol, 15% yield) as an off white solid. LCMS (ES+): m/z 717.2 [M+H]⁺

¹H NMR (400 MHZ, DMSO-d₆) δ 11.09 (s, 1H), 10.09 (s, 1H), 9.34 (s, 1H), 8.85 (s, 1H), 8.26 (d, J=2.3 Hz, 1H), 8.06-7.99 (m, 1H), 7.98-7.92 (m, 2H), 7.15 (s, 1H), 7.10-7.00 (m, 2H), 6.93-6.87 (m, 1H), 5.35 (dd, J=12.8, 5.4 Hz, 1H), 4.34 (d, J=4.3 Hz, 2H), 4.13 (s, 2H), 3.62 (d, J=11.6 Hz, 2H), 3.17-3.04 (m, 2H), 2.96-2.83 (m, 2H), 2.75-2.57 (m, 3H), 2.10-1.80 (m, 5H).

### 4-[[1-(2,6-dioxo-3-piperidyl)-3-methyl-2-oxo-benzimidazol-5-yl]amino]-N-[2-[[8-fluoro-6-hydroxy-7-(1,1,4-trioxo-1,2,5-thiadiazolidin-2-yl)-2-naphthyl]oxy]ethyl]benzamide (Example 126)

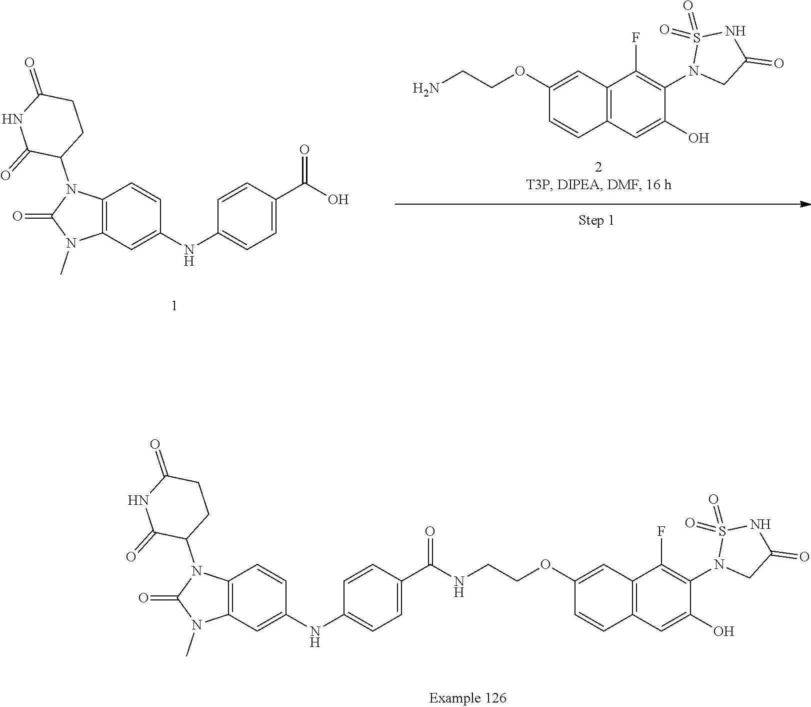 [SMILES: Cn1c(=O)n(C2CCC(=O)NC2=O)c2ccc(Nc3ccc(C(=O)O)cc3)cc21 ; NCCOc1ccc2cc(O)c(N3CC(=O)NS3(=O)=O)c(F)c2c1 ; Cn1c(=O)n(C2CCC(=O)NC2=O)c2ccc(Nc3ccc(C(=O)NCCOc4ccc5cc(O)c(N6CC(=O)NS6(=O)=O)c(F)c5c4)cc3)cc21]

### Step 1: 4-[[1-(2,6-dioxo-3-piperidyl)-3-methyl-2-oxo-benzimidazol-5-yl]amino]-N-[2-[[8-fluoro-6-hydroxy-7-(1,1,4-trioxo-1,2,5-thiadiazolidin-2-yl)-2-naphthyl]oxy]ethyl]benzamide (Example 126)

Into a 50 mL single neck round bottom flask containing a well-stirred solution of 4-[[1-(2,6-dioxo-3-piperidyl)-3-methyl-2-oxo-benzimidazol-5-yl]amino]benzoic acid (1, 140 mg, 354.99 μmol) in DMF (5 mL) was added DIPEA (229.40 mg, 1.77 mmol, 309.16 μL) and propylphosphonic anhydride (≥50 wt. % in ethyl acetate) (169.33 mg, 532.48 μmol, 50% purity). After 5 min. 5-[7-(2-aminoethoxy)-1-fluoro-3-hydroxy-2-naphthyl]-1,1-dioxo-1,2,5-thiadiazolidin-3-one (2, 151.37 mg, 425.99 μmol) was added. After 16 h, the volatiles were removed under reduced pressure and the residue was purified by reverse phase prep HPLC [Purification method: Column: X-select. C18 (19×150 mm), 5 mic; Mobile phase A: 0.1% TFA in water; Mobile phase B: MeCN] to afford 4-[[1-(2,6-dioxo-3-piperidyl)-3-methyl-2-oxo-benzimidazol-5-yl]amino]-N-[2-[[8-fluoro-6-hydroxy-7-(1,1,4-trioxo-1,2,5-thiadiazolidin-2-yl)-2-naphthyl]oxy]ethyl]benzamide (Example 126, 32 mg, 36.51 μmol, 10% yield, TFA salt) as a pale yellow solid. LCMS (ES+): m/z 732.2 [M+H]⁺

¹H NMR (400 MHZ, DMSO-d₆) δ 11.10 (s, 1H), 9.59 (s, 1H), 8.45-8.37 (m, 2H), 7.73 (d, J=8.5 Hz, 2H), 7.67 (d, J=9.0 Hz, 1H), 7.23 (d, J=2.5 Hz, 1H), 7.15 (dd, J=9.0, 2.5 Hz, 1H), 7.08-6.93 (m, 5H), 6.84 (dd, J=8.4, 2.1 Hz, 1H), 5.34 (dd, J=12.8, 5.4 Hz, 1H), 4.20 (t, J=5.9 Hz, 2H), 4.11 (s, 2H), 3.65 (q, J=5.8 Hz, 2H), 3.31 (s, 3H), 2.97-2.81 (m, 1H), 2.78-2.58 (m, 2H), 2.10-1.97 (m, 1H), 1.65-1.44 (m, 1H).

### 3-[5-[4-[2-[4-[8-fluoro-6-hydroxy-7-(1,1,4-trioxo-1,2,5-thiadiazolidin-2-yl)-2-naphthyl]-3,6-dihydro-2H-pyridin-1-yl]-2-oxo-ethyl]-1-piperidyl]-3-methyl-2-oxo-benzimidazol-1-yl]piperidine-2,6-dione (Example 127)

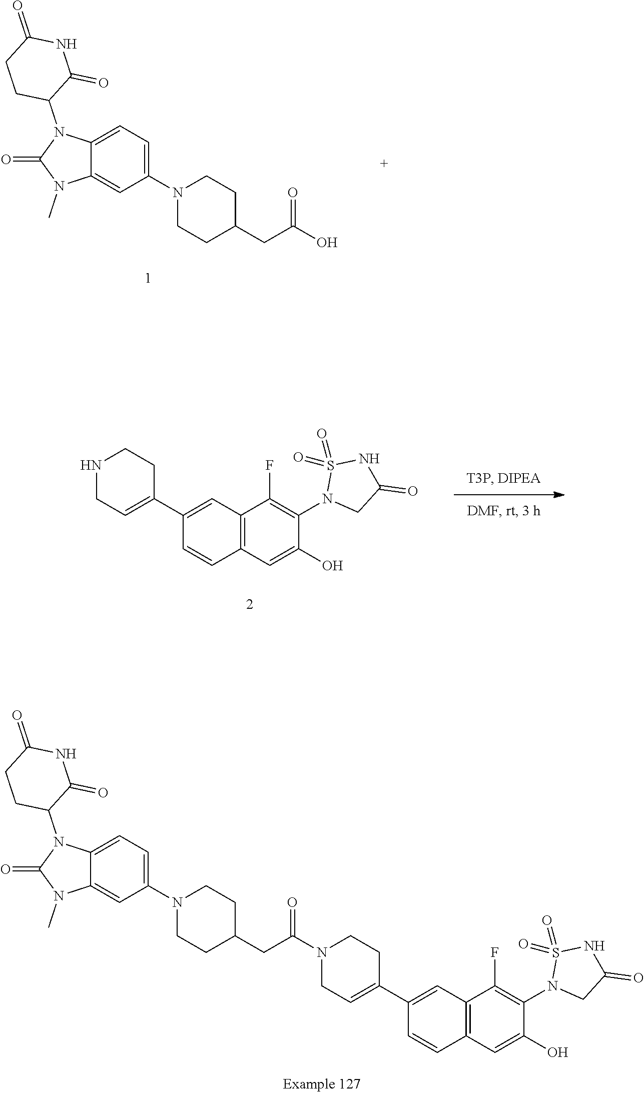 [SMILES: Cn1c(=O)n(C2CCC(=O)NC2=O)c2ccc(N3CCC(CC(=O)O)CC3)cc21 ; O=C1CN(c2c(O)cc3ccc(C4=CCNCC4)cc3c2F)S(=O)(=O)N1 ; Cn1c(=O)n(C2CCC(=O)NC2=O)c2ccc(N3CCC(CC(=O)N4CC=C(c5ccc6cc(O)c(N7CC(=O)NS7(=O)=O)c(F)c6c5)CC4)CC3)cc21]

### Step 1: 3-[5-[4-[2-[4-[8-fluoro-6-hydroxy-7-(1,1,4-trioxo-1,2,5-thiadiazolidin-2-yl)-2-naphthyl]-3,6-dihydro-2H-pyridin-1-yl]-2-oxo-ethyl]-1-piperidyl]-3-methyl-2-oxo-benzimidazol-1-yl]piperidine-2,6-dione (Example 127)

Into a 50 mL single neck round bottom flask containing a well-stirred solution of 2-[1-[1-(2,6-dioxo-3-piperidyl)-3-methyl-2-oxo-benzimidazol-5-yl]-4-piperidyl]acetic acid (1, 200 mg, 450.12 μmol) and 5-[1-fluoro-3-hydroxy-7-(1,2,3,6-tetrahydropyridin-4-yl)-2-naphthyl]-1,1-dioxo-1,2,5-thiadiazolidin-3-one (2, 195.97 mg, 495.13 μmol) in dry DMF (4 mL) were added 1-propanephosphonic anhydride (50 Wt % in EtOAc) (429.6 mg, 675.18 μmol) and DIPEA (174.52 mg, 1.35 mmol, 235.21 μL). After 3 h, the volatiles were removed under reduced pressure and the residue was purified by reverse-phase preparative HPLC [Column: Sunfire C18 (150×19) mm, 5 microns: Mobile phase A: 0.1% TFA in water and Mobile phase B: CH₃CN] to afford 3-[5-[4-[2-[4-[8-fluoro-6-hydroxy-7-(1,1,4-trioxo-1,2,5-thiadiazolidin-2-yl)-2-naphthyl]-3,6-dihydro-2H-pyridin-1-yl]-2-oxo-ethyl]-1-piperidyl]-3-methyl-2-oxo-benzimidazol-1-yl]piperidine-2,6-dione (Example 127, 55 mg, 60.49 μmol, 13% yield, TFA salt) as a red solid. LCMS (ES+): m/z 760.2 [M+H]⁺

¹H NMR (400 MHZ, DMSO-d₆) δ 7.81 (d, J=8.1 Hz, 1H), 7.77-7.69 (m, 2H), 7.22 (s, 2H), 7.08 (s, 1H), 6.42-6.34 (m, 1H), 5.40 (dd, J=12.9, 5.2 Hz, 1H), 4.29-4.23 (m, 3H), 4.20-4.13 (m, 1H), 3.78-3.71 (m, 3H), 3.59 (s, 2H), 3.37 (s, 3H), 2.96-2.84 (m, 1H), 2.79-2.62 (m, 2H), 2.63-2.55 (m, 1H), 2.23-2.09 (m, 1H), 2.07-1.95 (m, 4H), 1.71-1.53 (m, 3H).

### 3-[5-[1-[4-[[8-fluoro-6-hydroxy-7-(1,1,4-trioxo-1,2,5-thiadiazolidin-2-yl)-2-naphthyl]oxy]butyl]-4-piperidyl]-3-methyl-2-oxo-benzimidazol-1-yl]piperidine-2,6-dione (Example 128)

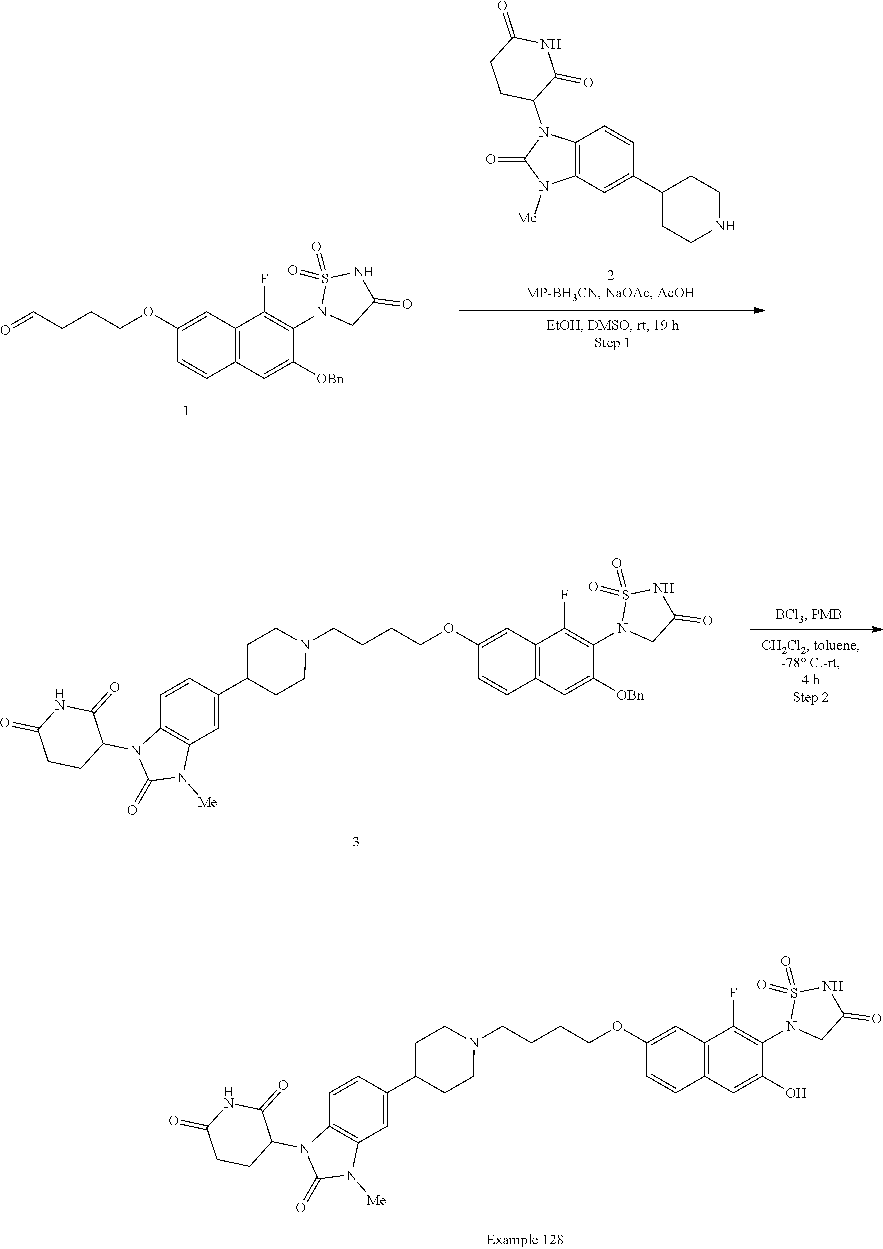 [SMILES: Cn1c(=O)n(C2CCC(=O)NC2=O)c2ccc(C3CCNCC3)cc21 ; O=CCCCOc1ccc2cc(OCc3ccccc3)c(N3CC(=O)NS3(=O)=O)c(F)c2c1 ; Cn1c(=O)n(C2CCC(=O)NC2=O)c2ccc(C3CCN(CCCCOc4ccc5cc(OCc6ccccc6)c(N6CC(=O)NS6(=O)=O)c(F)c5c4)CC3)cc21 ; Cn1c(=O)n(C2CCC(=O)NC2=O)c2ccc(C3CCN(CCCCOc4ccc5cc(O)c(N6CC(=O)NS6(=O)=O)c(F)c5c4)CC3)cc21]

### Step 1: 3-[5-[1-[4-[[6-benzyloxy-8-fluoro-7-(1,1,4-trioxo-1,2,5-thiadiazolidin-2-yl)-2-Step naphthyl]oxy]butyl]-4-piperidyl]-3-methyl-2-oxo-benzimidazol-1-yl]piperidine-2,6-dione (3)

Into a 100 mL sealed tube containing a well-stirred solution of 3-[3-methyl-2-oxo-5-(4-piperidyl)benzimidazol-1-yl]piperidine-2,6-dione (2, 330 mg, 723.03 μmol, TFA salt) in ethanol (7 mL), was added sodium acetate (237.24 mg, 2.89 mmol). After 10 min. acetic acid (434.18 mg, 7.23 mmol, 413.50 μL) and 4-[[6-benzyloxy-8-fluoro-7-(1,1,4-trioxo-1,2,5-thiadiazolidin-2-yl)-2-naphthyl]oxy]butanal (1, 341.62 mg, 723.03 μmol) in DMSO (3 mL) was added and stirred for 15 min. Subsequently. MP-cyanoborohydride (700 mg, 1.37 mmol). After 18 h. the reaction mixture was filtered and concentrated under reduced pressure and the residue was purified by reverse-phase preparative HPLC [Column SILIASEP C18 25μ, 120 g; Mobile phase A: 0.1% TFA in MQ-water; Mobile phase B: Acetonitrile] to obtain 3-[5-[1-[4-[[6-benzyloxy-8-fluoro-7-(1,1,4-trioxo-1,2,5-thiadiazolidin-2-yl)-2-naphthyl]oxy]butyl]-4-piperidyl]-3-methyl-2-oxo-benzimidazol-1-yl]piperidine-2,6-dione (3, 400 mg, 430.85 μmol, 60% yield, TFA salt) as an off-white solid. LCMS (ES+): m/z 799.2 [M+H]⁺

### Step 2: 3-[5-[1-[4-[[8-fluoro-6-hydroxy-7-(1,1,4-trioxo-1,2,5-thiadiazolidin-2-yl)-2-naphthyl]oxy]butyl]-4-piperidyl]-3-methyl-2-oxo-benzimidazol-1-yl]piperidine-2,6-dione (Example 128)

Into a 50 mL single neck round bottom flask containing a well-stirred solution of 3-[5-[1-[4-[[6-benzyloxy-8-fluoro-7-(1,1,4-trioxo-1,2,5-thiadiazolidin-2-yl)-2-naphthyl]oxy]butyl]-4-piperidyl]-3-methyl-2-oxo-benzimidazol-1-yl]piperidine-2,6-dione (3, 150 mg, 184.63 μmol) in DCM (3 mL) and toluene (3 mL) was added pentamethylbenzene (136.86 mg, 923.14 μmol, 149.24 μL). After 5 min, BCl₃ (1.0 M in DCM) (3.69 mmol, 3.7 mL) was added dropwise at −78° C. After the addition, the reaction mixture was stirred at rt for 4 h. The reaction mixture was cooled to −78° C. and quenched with 0.5 mL 5% MeOH/DCM solution. The reaction mixture was concentrated under reduced pressure at 35° C. and triturated with diethyl ether. The material was purified by reverse-phase preparative HPLC [Column: X-select C18 (150×19) mm 5 micron; Mobile phase A: 0.1% TFA in MQ-water; Mobile phase B: Acetonitrile] to afford 3-[5-[1-[4-[[8-fluoro-6-hydroxy-7-(1,1,4-trioxo-1,2,5-thiadiazolidin-2-yl)-2-naphthyl]oxy]butyl]-4-piperidyl]-3-methyl-2-oxo-benzimidazol-1-yl]piperidine-2,6-dione (Example 128, 24 mg, 28.94 μmol, 16% yield, TFA salt) as an off-white solid. LCMS (ES+): m/z 709.4 [M+H]⁺

¹H NMR (400 MHZ, DMSO-d₆) δ 11.10 (s, 1H), 9.51 (s, 1H), 9.15 (s, 1H), 7.69 (d, J=9.0 Hz, 1H), 7.21 (d, J=2.6 Hz, 1H), 7.15 (dd, J=9.0, 2.5 Hz, 1H), 7.10-7.01 (m, 3H), 6.91 (d, J=8.1 Hz, 1H), 5.35 (dd, J=12.8, 5.4 Hz, 1H), 4.17-4.13 (m, 2H), 4.09 (s, 2H), 3.63-3.58 (m, 2H), 3.24-3.15 (m, 2H), 3.12-2.98 (m, 2H), 2.98-2.81 (m, 2H), 2.76-2.58 (m, 2H), 2.05-1.96 (m, 3H), 1.95-1.80 (m, 6H). Note: additional peaks under solvent signal

### 2-[4-[1-(2,6-dioxo-3-piperidyl)-3-methyl-2-oxo-benzimidazol-5-yl]-3,3-difluoro-1-piperidyl]-N-[5-fluoro-7-hydroxy-6-(1,1,4-trioxo-1,2,5-thiadiazolidin-2-yl)-2-naphthyl]acetamide (Example 129)

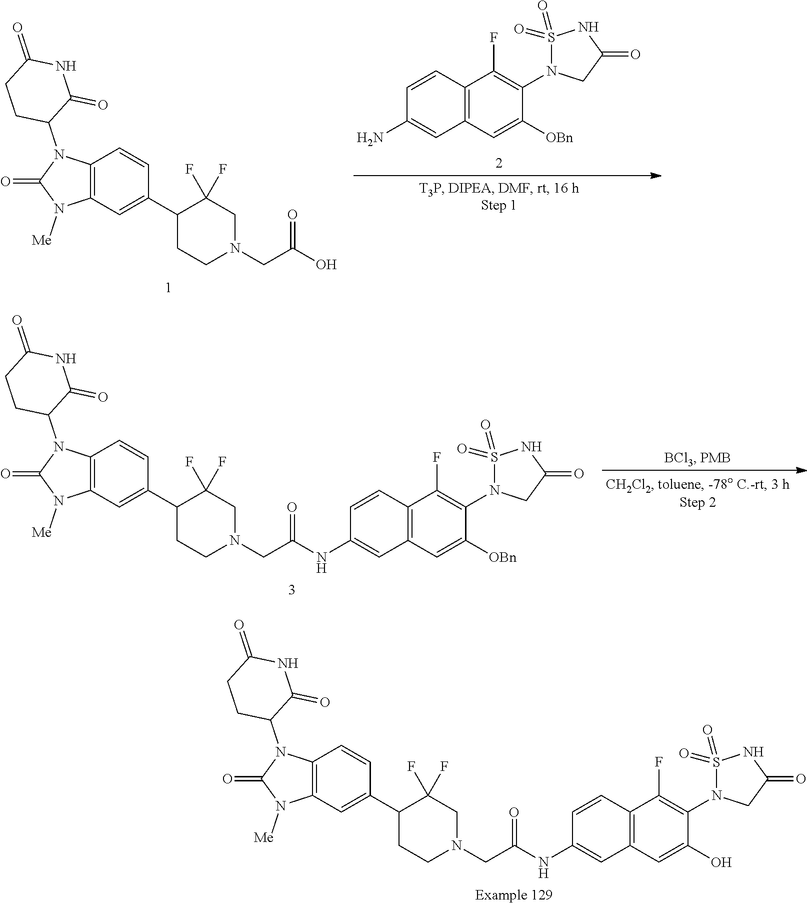 [SMILES: Cn1c(=O)n(C2CCC(=O)NC2=O)c2ccc(C3CCN(CC(=O)O)CC3(F)F)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(CC(=O)Nc4ccc5c(F)c(N6CC(=O)NS6(=O)=O)c(OCc6ccccc6)cc5c4)CC3(F)F)cc21 ; Cn1c(=O)n(C2CCC(=O)NC2=O)c2ccc(C3CCN(CC(=O)Nc4ccc5c(F)c(N6CC(=O)NS6(=O)=O)c(O)cc5c4)CC3(F)F)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3,3-difluoro-1-piperidyl]acetamide (3)

Into a 20 mL screw-capped vial containing a well-stirred solution of 2-[4-[1-(2,6-dioxo-3-piperidyl)-3-methyl-2-oxo-benzimidazol-5-yl]-3,3-difluoro-1-piperidyl]acetic acid (1, 300 mg, 534.13 μmol, TFA salt) and 5-(6-amino-3-benzyloxy-1-fluoro-2-naphthyl)-1,1-dioxo-1,2,5-thiadiazolidin-3-one (2, 290.01 mg, 534.13 μmol, TFA salt) in DMF (3 mL) was added DIPEA (690.32 mg, 5.34 mmol, 930.35 μL) followed by propylphosphonic anhydride (50 wt % in EtOAc) (679.80 mg, 1.07 mmol). After 16 h, the solvent was removed under reduced pressure and the residue was subjected to reverse-phase column chromatography [Column: RediSep gold-C18-120 g: Mobile Phase A: 0.1% TFA in water and Mobile Phase B: CH₃CN] to afford N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3,3-difluoro-1-piperidyl]acetamide (3, 260 mg, 270.88 μmol, 51% yield, TFA salt) as white solid. LCMS (ES+): m/z 820.2 [M+H]⁺

### Step 2: 2-[4-[1-(2,6-dioxo-3-piperidyl)-3-methyl-2-oxo-benzimidazol-5-yl]-3,3-difluoro-1-piperidyl]-N-[5-fluoro-7-hydroxy-6-(1,1,4-trioxo-1,2,5-thiadiazolidin-2-yl)-2-naphthyl]acetamide (Example 129)

Into a 25 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3,3-difluoro-1-piperidyl]acetamide (3, 210 mg, 249.22 μmol) in anhydrous DCM (2.5 mL) and anhydrous toluene (2.5 mL) was added pentamethylbenzene (184.73 mg, 1.25 mmol) at RT. The reaction mixture was cooled to −78° C. and BCl₃ (1.0 M solution in DCM) (6.0 mmol, 6 mL) was added dropwise over a period of 2 min. The reaction mixture was stirred at RT for 3 h. The reaction mixture was cooled to −78° C. and quenched slowly with 10% DCM in methanol (2 mL). The reaction mixture was concentrated under reduced pressure and purified by reverse-phase preparative HPLC [Column: ZORBAX-SB-C18, (21.2×250) mm, 7 μm; Mobile phase A: 0.1% TFA in water and Mobile phase B: CH₃CN] to afford 2-[4-[1-(2,6-dioxo-3-piperidyl)-3-methyl-2-oxo-benzimidazol-5-yl]-3,3-difluoro-1-piperidyl]-N-[5-fluoro-7-hydroxy-6-(1,1,4-trioxo-1,2,5-thiadiazolidin-2-yl)-2-naphthyl]acetamide (Example 129, 104 mg, 119.63 μmol, 48% yield, TFA salt) as an off-white solid. LCMS (ES+): m/z 730.1 [M+H]⁺

¹H NMR (400 MHZ, DMSO-d₆) δ 11.12 (s, 1H), 10.46 (s, 1H), 10.37 (s, 1H), 8.18 (d, J=1.8 Hz, 1H), 7.91 (d, J=9.0 Hz, 1H), 7.49 (dd, J=9.1, 2.0 Hz, 1H), 7.14 (s, 1H), 7.11 (d, J=8.2 Hz, 1H), 7.04-6.99 (m, 2H), 5.38 (dd, J=12.8, 5.4 Hz, 1H), 4.40 (s, 2H), 3.68-3.57 (m, 2H), 3.36 (s, 3H), 3.34-3.21 (m, 3H), 3.03-2.84 (m, 2H), 2.78-2.57 (m, 2H), 2.45-2.35 (m, 1H), 2.07-1.89 (m, 2H).

### 3-[5-[1-[2-[(3R)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-4-piperidyl]-3-methyl-2-oxo-benzimidazol-1-yl]piperidine-2,6-dione (Example 130)

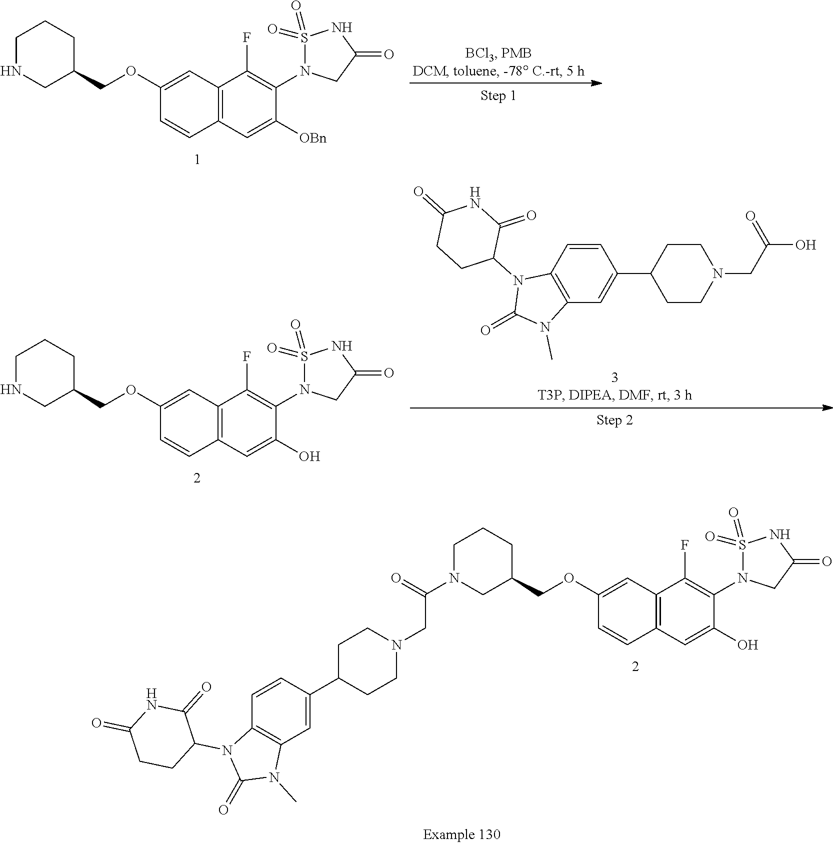 [SMILES: O=C1CN(c2c(OCc3ccccc3)cc3ccc(OC[C@@H]4CCCNC4)cc3c2F)S(=O)(=O)N1 ; O=C1CN(c2c(O)cc3ccc(OC[C@@H]4CCCNC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)N4CCC[C@@H](COc5ccc6cc(O)c(N7CC(=O)NS7(=O)=O)c(F)c6c5)C4)CC3)cc21]

### Step 1: 5-[1-fluoro-3-hydroxy-7-[[(3R)-3-piperidyl]methoxy]-2-naphthyl]-1,1-dioxo-1,2,5-thiadiazolidin-3-one (2)

Into a 100 mL single neck round bottom flask containing well-stirred solution of 5-[3-benzyloxy-1-fluoro-7-[[(3R)-3-piperidyl]methoxy]-2-naphthyl]-1,1-dioxo-1,2,5-thiadiazolidin-3-one (1, 500 mg, 1.00 mmol) and pentamethylbenzene (741.89 mg, 5 mmol) in anhydrous DCM (7.5 mL) and toluene (7.5 mL) was added boron trichloride (1.0 M in DCM) (2.35 g, 20.02 mmol, 20 mL) at −78° C. The reaction mixture was stirred at RT for 5 h. The reaction mixture was quenched with 5% MeOH in DCM (5 mL) at −78° C. The solvents were removed under reduced pressure and the residue was washed with diethyl ether (100 mL). The material was purified by reverse phase prep HPLC [Purification method: Column: Sunfire C18 (150×19) 5 micron; Mobile phase A: 0.1% TFA in water; Mobile phase B: MeCN] to obtain 5-[1-fluoro-3-hydroxy-7-[[(3R)-3-piperidyl]methoxy]-2-naphthyl]-1,1-dioxo-1,2,5-thiadiazolidin-3-one (2, 350 mg, 657.60 μmol, 66% yield, TFA salt) as pale brown solid. LCMS (ES+): m/z 410.0 [M+H]⁺

### Step 2: 3-[5-[1-[2-[(3R)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-4-piperidyl]-3-methyl-2-oxo-benzimidazol-1-yl]piperidine-2,6-dione (Example 130)

Into a 50 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (3, 150 mg, 288.66 μmol, TFA salt) in anhydrous DMF (3 mL) were added propylphosphonic anhydride solution (≥50) wt. % in ethyl acetate) (275.54 mg, 432.99 μmol, 50% purity) and DIPEA (222.60 mg, 1.72 mmol, 0.3 mL). After 10 min. 5-[1-fluoro-3-hydroxy-7-[[(3R)-3-piperidyl]methoxy]-2-naphthyl]-1,1-dioxo-1,2,5-thiadiazolidin-3-one (2, 184.05 mg, 375.25 μmol). After 3 h. water (25 mL) was added and the precipitate was filtered and purified by reverse phase prep HPLC [Purification method: Column: X-Select C18 (150×19) 5 micron; Mobile phase: 0.1% TFA in water; Mobile phase B: MeCN] to afford 3-[5-[1-[2-[(3R)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-4-piperidyl]-3-methyl-2-oxo-benzimidazol-1-yl]piperidine-2,6-dione (Example 130, 80 mg, 83.40 μmol, 29% yield, TFA salt) as a colorless solid. LCMS (ES+): m/z 792.0 [M+H]⁺

¹H NMR (400 MHZ, DMSO-d₆) δ 11.10 (s, 1H), 9.82 (s, 1H), 9.52-9.33 (m, 1H), 7.70 (dd, J=14.5, 9.1 Hz, 1H), 7.27-7.14 (m, 2H), 7.09-7.01 (m, 3H), 6.95-6.90 (m, 1H), 5.34 (dd, J=12.9, 5.3 Hz, 1H), 4.44-4.24 (m, 2H), 4.20 (s, 2H), 4.18-3.94 (m, 3H), 3.34 (d, J=1.9 Hz, 3H), 3.19-3.04 (m, 2H), 3.01-2.81 (m, 3H), 2.78-2.57 (m, 2H), 2.17-1.86 (m, 7H), 1.81-1.72 (m, 1H), 1.59-1.37 (m, 2H).

### 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 131)

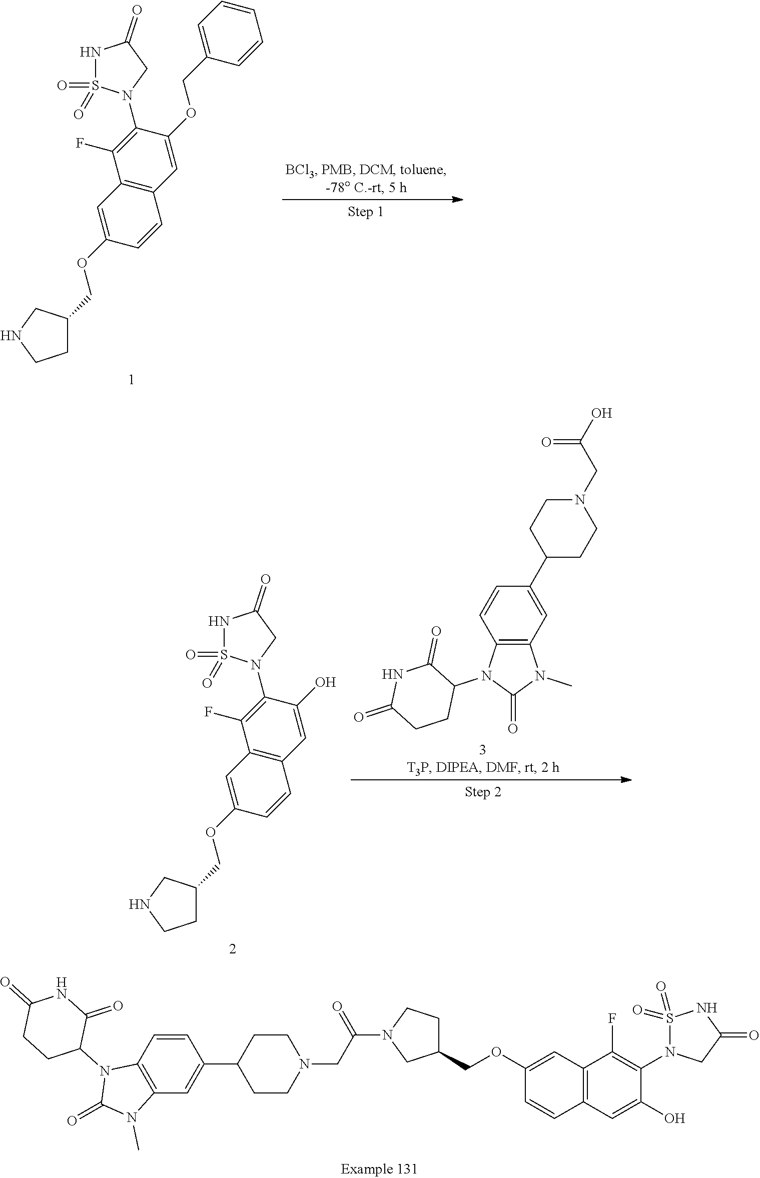 [SMILES: O=C1CN(c2c(OCc3ccccc3)cc3ccc(OC[C@@H]4CCNC4)cc3c2F)S(=O)(=O)N1 ; O=C1CN(c2c(O)cc3ccc(OC[C@@H]4CCNC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)N4CC[C@@H](COc5ccc6cc(O)c(N7CC(=O)NS7(=O)=O)c(F)c6c5)C4)CC3)cc21]

### Step 1: 5-[1-fluoro-3-hydroxy-7-[[(3R)-pyrrolidin-3-yl]methoxy]-2-naphthyl]-1,1-dioxo-1,2,5-thiadiazolidin-3-one (2)

To a 25 mL single neck round bottom flask containing a suspension of 5-[3-benzyloxy-1-fluoro-7-[[(3R)-pyrrolidin-3-yl]methoxy]-2-naphthyl]-1,1-dioxo-1,2,5-thiadiazolidin-3-one (1, 150 mg, 287.36 μmol, HCl salt) in a mixture of toluene (2.5 mL) and DCM (2.5 mL) was added pentamethylbenzene (213.01 mg, 1.44 mmol) under nitrogen atmosphere. The resulting suspension was cooled to −78° C. treated with boron trichloride (1.0 M in DCM) (673.41 mg, 5.75 mmol, 5.75 mL) via dropwise addition. The reaction mixture was stirred at ambient temperature. After 5 h, the reaction mixture was cooled to −78° C. and quenched with 5% MeOH in DCM (4 mL). The mixture was concentrated and the residue was triturated with diethyl ether (2×25 mL), filtered and dried under vacuum to obtain 5-[1-fluoro-3-hydroxy-7-[[(3R)-pyrrolidin-3-yl]methoxy]-2-naphthyl]-1,1-dioxo-1,2,5-thiadiazolidin-3-one (2, 125 mg, 281.36 μmol, 98% yield) as a brown solid. LCMS (ES+): m/z 396.0 [M+H]⁺

### Step 2: 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 131)

To a 25 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (3, 150 mg, 279.91 μmol, TFA salt) in DMF (1.5 mL) were added 1-propanephosphonic anhydride (50% in ethyl acetate) (267.19 mg, 419.87 μmol, 50% purity) and N,N-diisopropylethylamine (217.05 mg, 1.68 mmol, 292.53 μL). After 15 min, a solution of 5-[1-fluoro-3-hydroxy-7-[[(3R)-pyrrolidin-3-yl]methoxy]-2-naphthyl]-1,1-dioxo-1,2,5-thiadiazolidin-3-one (2, 124.36 mg, 279.91 μmol) in DMF (1.5 mL) was added. After 2 h, the solvent was removed under reduced pressure. The residue was triturated with ice water (25 mL), the precipitate was collected by filtration. The material was purified by reverse phase prep HPLC [Purification method: Column: X Select C18 (150×30) mm, 5 micron; Mobile Phase A: 0.1% TFA in water; Mobile phase B: MeCN] to obtain 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 131, 81 mg, 86.30 μmol, 31% yield, TFA salt) as a pale brown solid. LCMS (ES+): m/z 778.1 [M+H]⁺

¹H NMR (400 MHZ, DMSO-d₆) δ 11.10 (s, 1H), 9.79 (s, 1H), 9.56 (s, 1H), 7.77-7.67 (m, 1H), 7.23 (dd, J=6.2, 2.6 Hz, 1H), 7.19-7.14 (m, 1H), 7.09-7.03 (m, 3H), 6.95-6.91 (m, 1H), 5.36 (dd, J=12.8, 5.4 Hz, 1H), 4.27-4.18 (m, 4H), 4.15-4.03 (m, 2H), 3.71-3.54 (m, 3H), 3.50-3.38 (m, 2H), 3.35 (s, 4H), 3.22-3.06 (m, 2H), 3.00-2.80 (m, 3H), 2.78-2.58 (m, 2H), 2.25-2.14 (m, 1H), 2.14-2.05 (m, 2H), 2.03-1.78 (m, 5H).

### N-[[4-[[1-(2,6-dioxo-3-piperidyl)-3-methyl-2-oxo-benzimidazol-5-yl]amino]phenyl]-5-fluoro-7-hydroxy-6-(1,1,4-trioxo-1,2,5-thiadiazolidin-2-yl)naphthalene-2-carboxamide (Example 132)

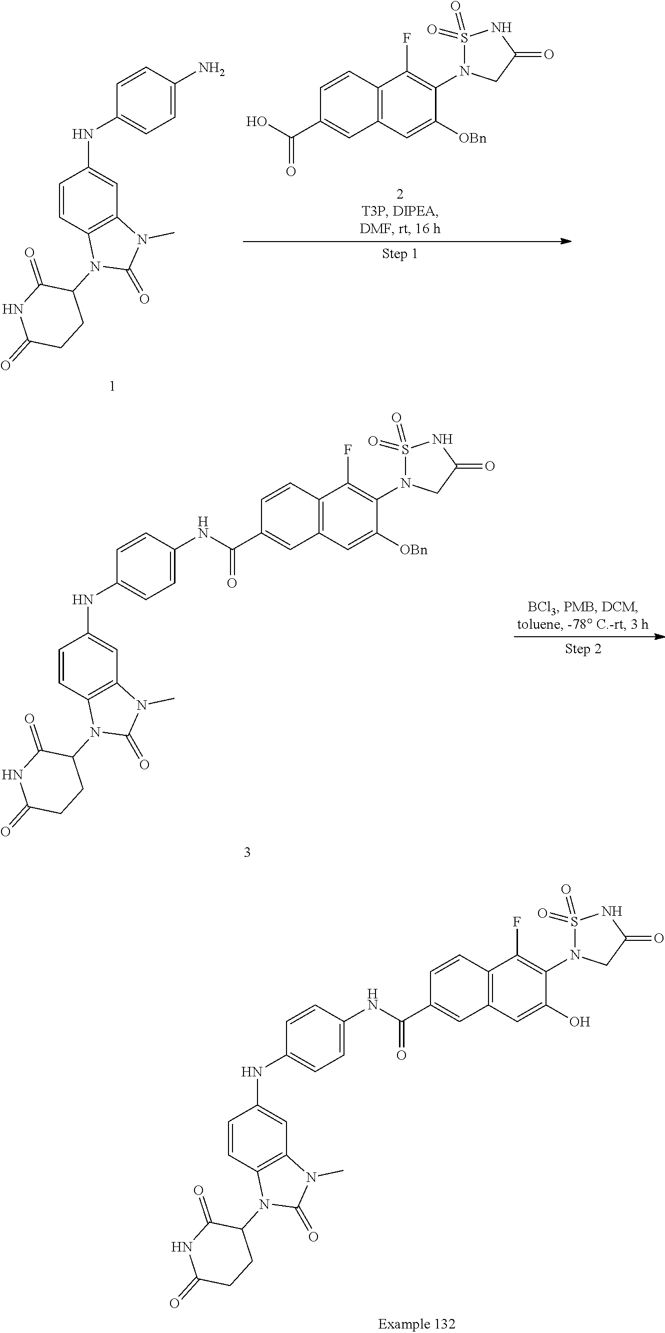 [SMILES: Cn1c(=O)n(C2CCC(=O)NC2=O)c2ccc(Nc3ccc(N)cc3)cc21 ; O=C1CN(c2c(OCc3ccccc3)cc3cc(C(=O)O)ccc3c2F)S(=O)(=O)N1 ; Cn1c(=O)n(C2CCC(=O)NC2=O)c2ccc(Nc3ccc(NC(=O)c4ccc5c(F)c(N6CC(=O)NS6(=O)=O)c(OCc6ccccc6)cc5c4)cc3)cc21 ; Cn1c(=O)n(C2CCC(=O)NC2=O)c2ccc(Nc3ccc(NC(=O)c4ccc5c(F)c(N6CC(=O)NS6(=O)=O)c(O)cc5c4)cc3)cc21]

### Step 1: 7-benzyloxy-N-[[4-[[1-(2,6-dioxo-3-piperidyl)-3-methyl-2-oxo-benzimidazol-5-yl]amino]phenyl]-5-fluoro-6-(1,1,4-trioxo-1,2,5-thiadiazolidin-2-yl)naphthalene-2-carboxamide (3)

Into a 20 mL vial containing a well-stirred solution of 7-benzyloxy-5-fluoro-6-(1,1,4-trioxo-1,2,5-thiadiazolidin-2-yl)naphthalene-2-carboxylic acid (2, 200 mg, 450.74 μmol) in DMF (5 mL) were added T3P 50% in ethyl acetate (215.12 mg, 676.11 μmol, 0.43 mL) and N,N-diisopropylethylamine (291.27 mg, 2.25 mmol, 392.55 μL). After stirring for 5 min at RT 3-[5-(4-aminoanilino)-3-methyl-2-oxo-benzimidazol-1-yl]piperidine-2,6-dione (1, 276.39 mg, 495.81 μmol, TFA salt) was added. Then reaction mixture was stirred at 70° C. After 16 h, the reaction mixture was concentrated under vacuum and the residue was suspended in water (50 mL). The separated solid was filtered and washed with water, diethyl ether and dried to afford 7-benzyloxy-N-[4-[[1-(2,6-dioxo-3-piperidyl)-3-methyl-2-oxo-benzimidazol-5-yl]amino]phenyl]-5-fluoro-6-(1,1,4-trioxo-1,2,5-thiadiazolidin-2-yl)naphthalene-2-carboxamide (3, 350 mg, 303.80 μmol, 67.40% yield, 68% purity) as a grey solid. LCMS (ES+): m/z 778.2 [M+H]⁺

### Step 2: N-[[4-[[1-(2,6-dioxo-3-piperidyl)-3-methyl-2-oxo-benzimidazol-5-yl]amino]phenyl]-5-fluoro-7-hydroxy-6-(1,1,4-trioxo-1,2,5-thiadiazolidin-2-yl)naphthalene-2-carboxamide (Example 132)

Into a 50 mL round bottom flask containing a well-stirred solution of 7-benzyloxy-N-[[4-[[1-(2,6-dioxo-3-piperidyl)-3-methyl-2-oxo-benzimidazol-5-yl]amino]phenyl]-5-fluoro-6-(1,1,4-trioxo-1,2,5-thiadiazolidin-2-yl)naphthalene-2-carboxamide (3, 300 mg, 260.40 μmol) in toluene (3 mL) and DCM (3 mL) was added pentamethylbenzene (193.01 mg, 1.30 mmol, 210.48 μL) and the reaction mixture was cooled to −78° C. Then, boron trichloride (1.0 M in DCM) (610.21 mg, 5.21 mmol, 5.2 mL) was added dropwise over a period of 2 min. Subsequently the reaction mixture was brought to RT. After 4 h. the reaction mixture was cooled to −78° C. and quenched with 10% MeOH in DCM (3 mL). The reaction mixture was concentrated under reduced pressure and the residue was triturated with diethyl ether (10 mL), filtered and purified by reverse phase prep HPLC [Purification method: Column: X-bridge, C18 (150×19) mm, 5 micron; Mobile phase A: 0.1% TFA in water and Mobile phase B: MeCN] to afford N-[[4-[[1-(2,6-dioxo-3-piperidyl)-3-methyl-2-oxo-benzimidazol-5-yl]amino]phenyl]-5-fluoro-7-hydroxy-6-(1,1,4-trioxo-1,2,5-thiadiazolidin-2-yl)naphthalene-2-carboxamide (Example 132, 70 mg, 82.66 μmol, 32% yield, TFA salt) as a yellow solid. LCMS (ES+): m/z 688.0 [M+H]⁺

¹H NMR (400 MHZ, DMSO-d₆) δ 11.09 (s, 1H), 10.26 (s, 1H), 8.39 (s, 1H), 7.91-7.84 (m, 1H), 7.63 (d, J=8.6 Hz, 2H), 7.27 (s, 1H), 7.07-6.97 (m, 3H), 6.90 (d, J=2.0 Hz, 1H), 6.77 (dd, J=8.4, 2.1 Hz, 1H), 5.31 (dd, J=12.9, 5.4 Hz, 1H), 4.53 (s, 2H), 3.30 (s, 3H), 2.97-2.84 (m, 1H), 2.77-2.58 (m, 2H), 2.05-1.97 (m, 1H).

### 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 133)

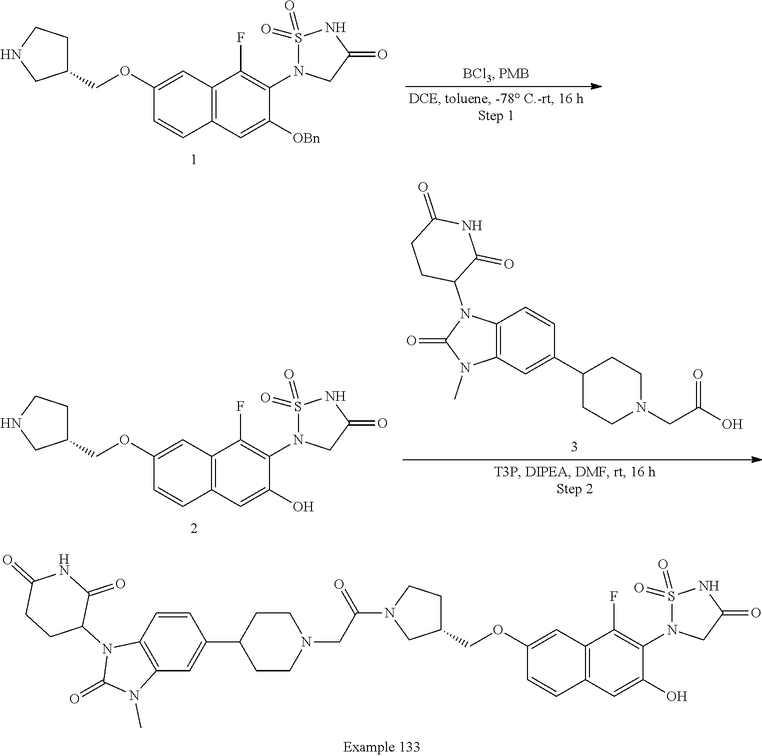 [SMILES: O=C1CN(c2c(OCc3ccccc3)cc3ccc(OC[C@H]4CCNC4)cc3c2F)S(=O)(=O)N1 ; O=C1CN(c2c(O)cc3ccc(OC[C@H]4CCNC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)N4CC[C@H](COc5ccc6cc(O)c(N7CC(=O)NS7(=O)=O)c(F)c6c5)C4)CC3)cc21]

### Step 1: 5-[1-fluoro-3-hydroxy-7-[[(3S)-pyrrolidin-3-yl]methoxy]-2-naphthyl]-1,1-dioxo-1,2,5-thiadiazolidin-3-one (2)

Into a 100 mL single neck round bottom flask containing a well-stirred solution of 5-[3-benzyloxy-1-fluoro-7-[[(3S)-pyrrolidin-3-yl]methoxy]-2-naphthyl]-1,1-dioxo-1,2,5-thiadiazolidin-3-one (1, 300 mg, 574.73 μmol, HCl salt) in a 1:1 mixture of anhydrous DCE (2 mL) and toluene (1 mL) was added pentamethylbenzene (255.60 mg, 1.72 mmol) under nitrogen atmosphere. The reaction mixture was cooled to −78° C. and BCl₃ (1.0 M solution in DCM) (1.92 mL, 1.92 mmol) was added dropwise. The reaction mixture was stirred at RT for 16 h. The reaction mixture was cooled to −78° C. and quenched with 3% MeOH in DCM (2 mL). The volatiles were removed under reduced pressure and the residue was purified by reverse-phase preparative HPLC [Column: Sylicycle C18 (150×19) mm, 25 μm; Mobile Phase A: 0.1% TFA in water and CH₃CN] to afford 5-[1-fluoro-3-hydroxy-7-[[(3S)-pyrrolidin-3-yl]methoxy]-2-naphthyl]-1,1-dioxo-1,2,5-thiadiazolidin-3-one (2, 180 mg, 350.55 μmol, 61% yield, TFA salt) as a brown solid. LCMS (ES+): m/z 396.0 [M+H]⁺

### Step 2: 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 133)

Into a 25 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (3, 140 mg, 262.07 μmol, TFA salt) in anhydrous DMF (5 mL) were added DIPEA (101.61 mg, 786.20 μmol, 136.94 μL) and propylphosphonic anhydride solution (50 Wt. % in EtOAc) (250.15 mg, 393.10 μmol). The reaction mixture was stirred at RT for 1 h before addition of 5-[1-fluoro-3-hydroxy-7-[[(3S)-pyrrolidin-3-yl]methoxy]-2-naphthyl]-1,1-dioxo-1,2,5-thiadiazolidin-3-one (2, 121.12 mg, 235.86 μmol, TFA salt). After 15 h, the volatiles were removed under reduced pressure and the residue was purified by reverse-phase preparative HPLC [Column: X-Select-C18 (150×19) 5 μm; Mobile Phase A: 0.1% TFA in water and Mobile Phase B: CH₃CN] to obtain 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 133, 108 mg, 44% yield, TFA salt) as a white solid. LCMS (ES+): m/z 778.2 [M+H]⁺

¹H NMR (400 MHZ, DMSO-d₆) δ 11.10 (s, 1H), 9.73 (s, 1H), 9.55 (s, 1H), 7.74-7.67 (m, 1H), 7.23 (dd, J=6.5, 2.5 Hz, 1H), 7.19-7.13 (m, 1H), 7.10-7.03 (m, 3H), 6.96-6.89 (m, 1H), 5.36 (dd, J=12.7, 5.4 Hz, 1H), 4.27-4.17 (m, 4H), 4.16-4.03 (m, 2H), 3.35 (s, 3H), 3.21-3.08 (m, 2H), 2.97-2.80 (m, 3H), 2.76-2.57 (m, 2H), 2.24-1.76 (m, 7H).

### 3-[5-[1-[2-[(3S)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-4-piperidyl]-3-methyl-2-oxo-benzimidazol-1-yl]piperidine-2,6-dione (Example 134)

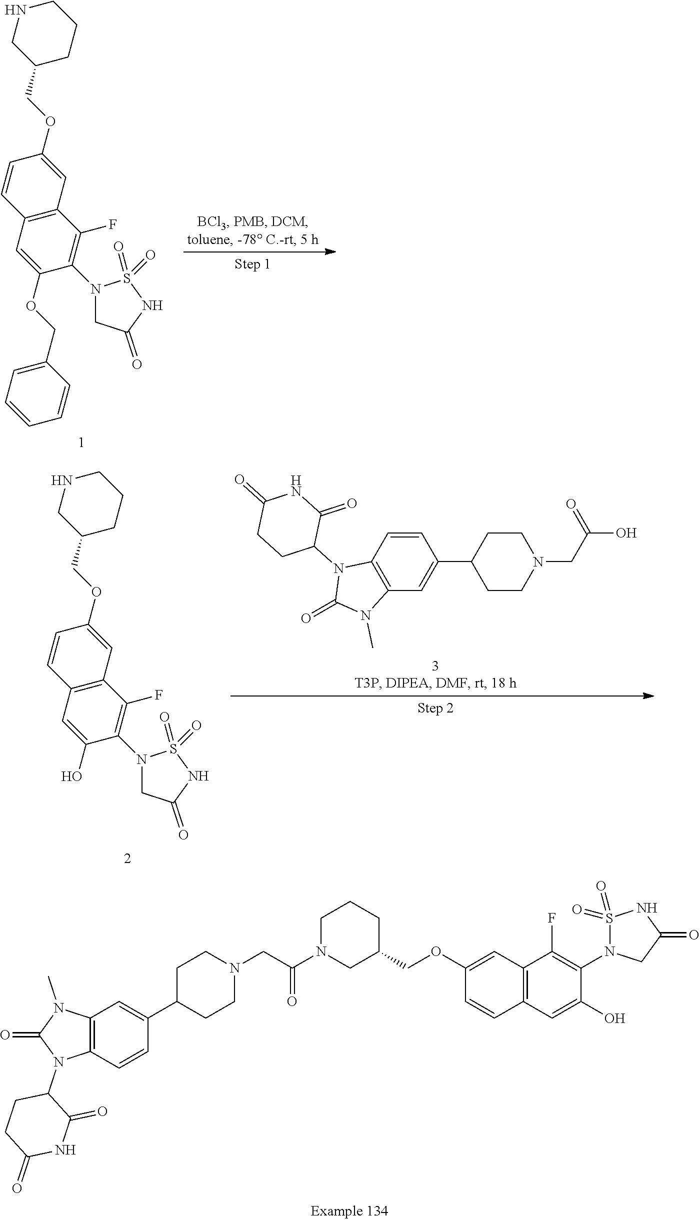 [SMILES: O=C1CN(c2c(OCc3ccccc3)cc3ccc(OC[C@H]4CCCNC4)cc3c2F)S(=O)(=O)N1 ; O=C1CN(c2c(O)cc3ccc(OC[C@H]4CCCNC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)N4CCC[C@H](COc5ccc6cc(O)c(N7CC(=O)NS7(=O)=O)c(F)c6c5)C4)CC3)cc21]

### Step 1: 5-[1-fluoro-3-hydroxy-7-[[(3S)-3-piperidyl]methoxy]-2-naphthyl]-1,1-dioxo-1,2,5-thiadiazolidin-3-one (2)

Into a 50 mL single neck round bottom flask containing a well-stirred solution of 5-[3-benzyloxy-1-fluoro-7-[[(3S)-3-piperidyl]methoxy]-2-naphthyl]-1,1-dioxo-1,2,5-thiadiazolidin-3-one (1, 100 mg, 200.18 μmol) in a mixture of anhydrous toluene (2.5 mL) and anhydrous DCM (2.5 mL) was added pentamethylbenzene (178.05 mg, 1.20 mmol) at RT, and the resultant reaction mixture was cooled to cooled to −78° C. Then BCl₃ (1.0 M in DCM) (351.5 mg, 3.00 mmol, 3 mL) was added dropwise. The reaction mixture was brought to RT. After 5 h, the reaction mixture was quenched with 5% MeOH in DCM (3 mL) at −78° C. The solvents were removed under reduced pressure and the residue triturated with Et₂O (15 mL). The material was purified by reverse phase prep HPLC [Purification method: Column: Sunfire C18 (150 mm×19 mm) 5 micron; Mobile phase A: 0.1% TFA in water and Mobile phase B: MeCN] to afford 5-[1-fluoro-3-hydroxy-7-[[(3S)-3-piperidyl]methoxy]-2-naphthyl]-1,1-dioxo-1,2,5-thiadiazolidin-3-one (2, 35 mg, 65.99 μmol, 33% yield, TFA salt) as a colorless solid. LCMS (ES+): m/z 410.0 [M+H]⁺

¹H NMR (400 MHZ, DMSO-d6): δ 9.53 (bs, 1H), 8.43 (bs, 1H), 7.70 (d, J=8.80 Hz, 1H), 7.21 (d, J=2.40 Hz, 1H), 7.15 (dd, J=2.40, 8.80 Hz, 1H), 7.04 (s, 1H), 4.10-3.98 (m, 4H), 3.42-3.34 (m, 1H), 3.25-2.87 (m, 1H), 2.86-2.78 (m, 2H), 2.33-2.25 (m, 1H), 1.91-1.83 (m, 2H), 1.66-1.63 (m, 1H), 1.41-1.38 (m, 1H).

### Step 2: 3-[5-[1-[2-[(3S)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-4-piperidyl]-3-methyl-2-oxo-benzimidazol-1-yl]piperidine-2,6-dione (Example 134)

Into a 50 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (3, 173.78 mg, 432.26 μmol) in anhydrous DMF (4 mL) were added DIPEA (1.48 g, 11.48 mmol, 2 mL) and propylphosphonic anhydride (≥50 wt. % in EtOAc) (412.61 mg, 648.39 μmol, 50% purity). After 10 min, 5-[1-fluoro-3-hydroxy-7-[[(3S)-3-piperidyl]methoxy]-2-naphthyl]-1,1-dioxo-1,2,5-thiadiazolidin-3-one (2, 200 mg, 432.26 μmol). After 18 h, the reaction mixture was concentrated under reduced pressure and purified by reverse phase preparatory HPLC [Purification method: Column: X-Bridge C18 (150 mm×19 mm) 5 micron; Mobile phase A: 0.1% TFA in water and Mobile phase B: MeCN] to afford 3-[5-[1-[2-[(3S)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-4-piperidyl]-3-methyl-2-oxo-benzimidazol-1-yl]piperidine-2,6-dione (Example 134, 29 mg, 30.80 μmol, 7% yield, TFA salt) as a colorless solid. LCMS (ES+): m/z 792.0 [M+H]⁺

¹H NMR (400 MHZ, DMSO-d₆) δ 11.10 (s, 1H), 9.81 (s, 1H), 9.40 (s, 1H), 7.76-7.63 (m, 1H), 7.28-7.14 (m, 2H), 7.10-7.01 (m, 3H), 6.95-6.88 (m, 1H), 5.35 (dd, J=12.9, 5.3 Hz, 1H), 4.45-4.24 (m, 2H), 4.21 (s, 2H), 4.17-3.93 (m, 3H), 3.35 (s, 3H), 3.20-3.05 (m, 3H), 3.01-2.80 (m, 4H), 2.75-2.57 (m, 2H), 2.17-1.85 (m, 7H), 1.82-1.70 (m, 1H).

### 3-[5-[1-[2-[4-[8-fluoro-6-hydroxy-7-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (Example 135)

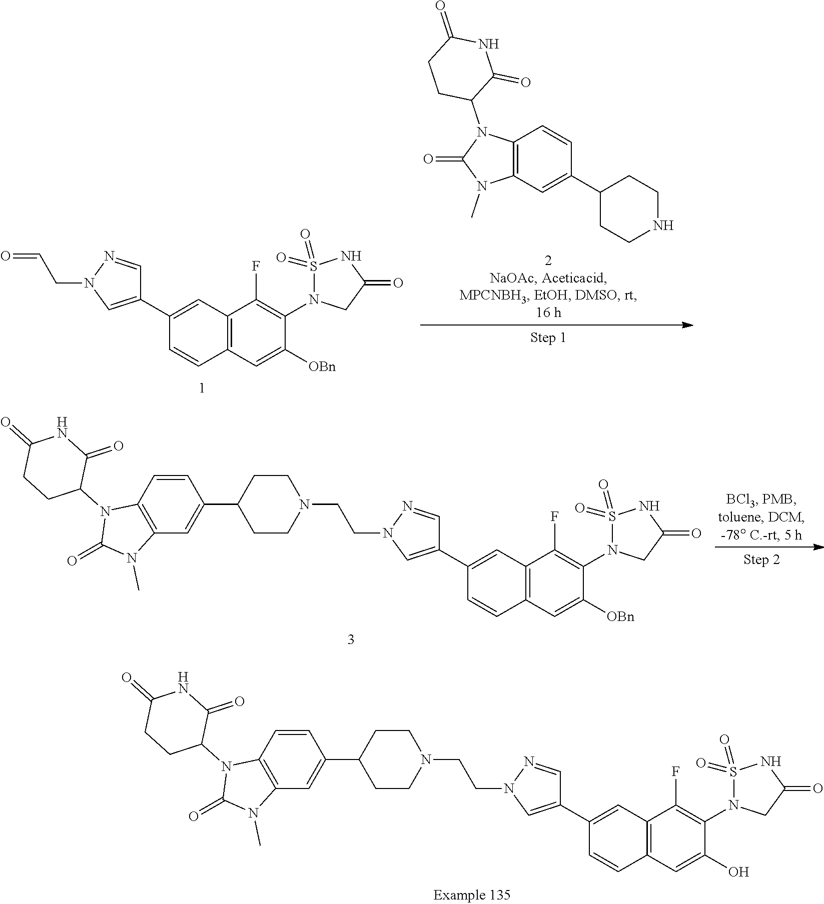 [SMILES: O=CCn1cc(-c2ccc3cc(OCc4ccccc4)c(N4CC(=O)NS4(=O)=O)c(F)c3c2)cn1 ; Cn1c(=O)n(C2CCC(=O)NC2=O)c2ccc(C3CCNCC3)cc21 ; Cn1c(=O)n(C2CCC(=O)NC2=O)c2ccc(C3CCN(CCn4cc(-c5ccc6cc(OCc7ccccc7)c(N7CC(=O)NS7(=O)=O)c(F)c6c5)cn4)CC3)cc21 ; Cn1c(=O)n(C2CCC(=O)NC2=O)c2ccc(C3CCN(CCn4cc(-c5ccc6cc(O)c(N7CC(=O)NS7(=O)=O)c(F)c6c5)cn4)CC3)cc21]

### Step 1: 3-[5-[1-[2-[4-[6-benzyloxy-8-fluoro-7-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (3)

In a 50 mL single neck round bottom flask a well-stirred solution of 3-[3-methyl-2-oxo-5-(4-piperidyl)benzimidazol-1-yl]piperidine-2,6-dione (2, 148.10 mg, 292.03 μmol, TFA salt) in ethanol (2 mL) and DMSO (2 mL) were added 2-[4-[6-benzyloxy-8-fluoro-7-(1,1,4-trioxo-1,2,5-thiadiazolidin-2-yl)-2-naphthyl]pyrazol-1-yl]acetaldehyde (1, 350 mg, 389.29 μmol), sodium acetate (95.80 mg, 1.17 mmol, 62.61 μL) and acetic acid (215.03 mg, 3.58 mmol, 204.79 μL). After 5 min, MP-cyanoborohydride (2 mmol in 1 g) (700 mg, 1.4 mmol). Upon completion, the reaction mixture was filtered through Celite and washed with ethanol (20 mL). The filtrate was concentrated under reduced pressure and subjected to reverse phase column chromatography [Purification method: Siliasep premium C18, 25 μm, 120 g column; Mobile phase A: 0.1% TFA in water; Mobile phase B: Acetonitrile] to afford 3-[5-[1-[2-[4-[6-benzyloxy-8-fluoro-7-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (3, 160 mg, 154.03 μmol, 40% yield, TFA salt) as an off-white solid. LCMS (ES+): m/z 821.1 [M+H]⁺

### Step 2: 3-[5-[1-[2-[4-[8-fluoro-6-hydroxy-7-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (Example 135)

Into a 50 mL single neck round bottom flask containing a well-stirred solution of 3-[5-[1-[2-[4-[6-benzyloxy-8-fluoro-7-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (3, 130 mg, 139.05 μmol) in toluene (2.5 mL) and DCM (2.5 mL) was added pentamethylbenzene (103.07 mg, 695.25 μmol, 112.40 μL). The reaction mixture was cooled to −78° C. and treated with boron trichloride (1.0 M in DCM) (2.78 mmol, 2.7 mL). The reaction mixture was stirred at RT for 5 h. The reaction was quenched with 5% MeOH in DCM (1.5 mL) at −75° C., concentrated under reduced pressure and the residue was triturated with diethyl ether (10 mL). The material was purified by reverse phase prep HPLC [Purification method: Column: Zorbax C18 (250×21.2) 7 micron; Mobile phase A: 0.1% TFA in water, Mobile phase B: MeCN] to afford 3-[5-[1-[2-[4-[8-fluoro-6-hydroxy-7-(1,1,4-trioxo-1,2,5-thiadiazolidin-2-yl)-2-naphthyl]pyrazol-1-yl]ethyl]-4-piperidyl]-3-methyl-2-oxo-benzimidazol-1-yl]piperidine-2,6-dione (Example 135, 21 mg, 23.00 μmol, 17% yield, TFA salt) as an off white solid. LCMS (ES+): m/z 731.1 [M+H]⁺

¹H NMR (400 MHZ, DMSO-d₆) δ 11.10 (s, 1H), 9.94 (s, 1H), 9.31 (s, 1H), 8.46 (s, 1H), 8.18 (s, 1H), 8.05 (s, 1H), 7.82-7.72 (m, 2H), 7.10-7.02 (m, 3H), 6.94-6.88 (m, 1H), 5.35 (dd, J=12.8, 5.4 Hz, 1H), 4.64 (t, J=6.3 Hz, 2H), 4.19-4.15 (m, 2H), 3.68 (d, J=14.3 Hz, 4H), 3.34 (s, 3H), 3.24-3.10 (m, 2H), 2.95-2.82 (m, 2H), 2.76-2.57 (m, 2H), 2.10-1.82 (m, 5H).

### 3-(5-(1-(2-(3-((7-(1,1-dioxido-4-oxo-1,2,5-thiadiazolidin-2-yl)-8-fluoro-6-hydroxynaphthalen-2-yl)oxy)azetidin-1-yl)-2-oxoethyl)piperidin-4-yl)-1-methyl-1H-indazol-3-yl)piperidine-2,6-dione (Example 136)

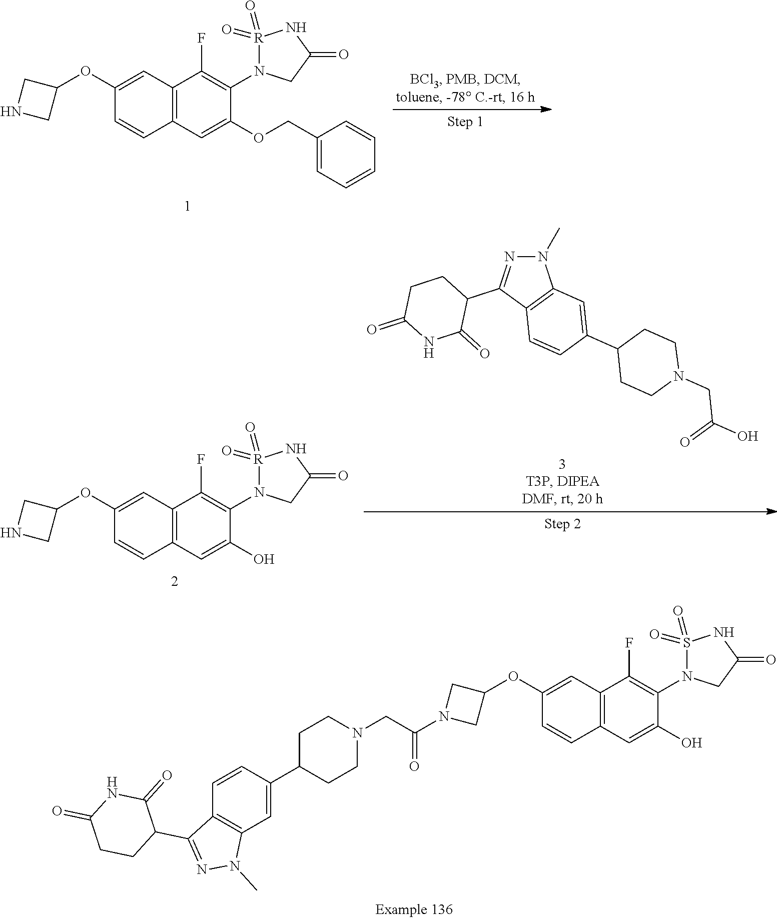

### Step 1: 5-[7-(azetidin-3-yloxy)-1-fluoro-3-hydroxy-2-naphthyl]-1,1-dioxo-1,2,5-thiadiazolidin-3-one (2)

Into a 25 mL single neck, round bottom flask containing a well-stirred solution of 5-[7-(azetidin-3-yloxy)-3-benzyloxy-1-fluoro-2-naphthyl]-1,1-dioxo-1,2,5-thiadiazolidin-3-one (1, 500 mg, 1.01 mmol, HCl salt) and pentamethylbenzene (900.39 mg, 6.07 mmol) in a mixture of dry toluene (2.5 mL) and dry DCM (2.5 mL) under nitrogen atmosphere at −78° C. was added BCl₃ (1.0 M in DCM, 10.12 mmol, 10.12 mL) via dropwise addition. The mixture was stirred at RT for 16 h. The reaction was quenched at −78° C. with 10% DCM in MeOH (2 mL), concentrated under reduced pressure and purified by using reverse-phase preparative HPLC [Column: Silycycle C18 (150×19) mm 5 μm; Mobile phase A: 0.1% TFA and Mobile Phase B: afford 5-[7-(azetidin-3-yloxy)-1-fluoro-3-hydroxy-2-naphthyl]-1,1-dioxo-1,2,5-thiadiazolidin-3-one (2, 230 mg, 432.17 μmol, 43% yield, TFA salt) as a brown solid. LCMS (ES+): m/z 368 [M+H]⁺

### Step 2: 3-(5-(1-(2-(3-((7-(1,1-dioxido-4-oxo-1,2,5-thiadiazolidin-2-yl)-8-fluoro-6-hydroxynaphthalen-2-yl)oxy)azetidin-1-yl)-2-oxoethyl)piperidin-4-yl)-1-methyl-1H-indazol-3-yl)piperidine-2,6-dione (Example 136)

In to a 20 mL vial containing a well-stirred solution of 2-[4-[3-(2,6-dioxo-3-piperidyl)-1-methyl-indazol-6-yl]-1-piperidyl]acetic acid (3, 104.19 mg, 205.66 μmol, TFA salt) in DMF (2 mL) were added DIPEA (120.82 mg, 934.82 μmol, 162.83 μL) and 1-propanephosphonic anhydride (50 Wt % in EtOAc) (178.47 mg, 280.45 μmol). After 15 min, 5-[7-(azetidin-3-yloxy)-1-fluoro-3-hydroxy-2-naphthyl]-1,1-dioxo-1,2,5-thiadiazolidin-3-one (2, 99.50 mg, 186.96 μmol) was added. After 16 h, the reaction mixture was concentrated under reduced pressure and purified via reverse-phase preparative HPLC [Column: X-Select-C18 (150×19) 5 μm; Mobile phase: 0.1% TFA in water and Mobile Phase B: CH₃CN) to obtain 3-(5-(1-(2-(3-((7-(1,1-dioxido-4-oxo-1,2,5-thiadiazolidin-2-yl)-8-fluoro-6-hydroxynaphthalen-2-yl)oxy)azetidin-1-yl)-2-oxoethyl)piperidin-4-yl)-1-methyl-1H-indazol-3-yl)piperidine-2,6-dione (Example 136, 94 mg, 106.36 μmol, 57% yield, TFA salt) as a white solid. LCMS (ES+): m/z 735.0 [M+H]⁺

¹H NMR (400 MHZ, DMSO-d₆) δ 10.90 (s, 1H), 9.85 (s, 1H), 9.75 (s, 1H), 7.76 (d, J=9.0 Hz, 1H), 7.67 (d, J=8.4 Hz, 1H), 7.42 (s, 1H), 7.19 (dd, J=9.0, 2.5 Hz, 1H), 7.07 (s, 1H), 7.05 (d, 1H), 7.00 (d, J=2.6 Hz, 1H), 5.34-5.22 (m, 1H), 4.79-4.69 (m, 1H), 4.53 (dd, J=10.7, 6.4 Hz, 1H), 4.34 (dd, J=9.8, 5.0 Hz, 1H), 4.28-4.22 (m, 2H), 4.20 (d, J=3.3 Hz, 2H), 4.14-4.09 (m, 2H), 4.07-4.01 (m, 1H), 4.00-3.96 (m, 3H), 3.21-3.08 (m, 2H), 3.03-2.90 (m, 1H), 2.65-2.55 (m, 2H), 2.46-2.34 (m, 1H), 2.21-2.00 (m, 5H).

### 3-[5-[1-[2-[(3R)-3-[[8-fluoro-6-hydroxy-7-(1,1,4-trioxo-1,2,5-thiadiazolidin-2-yl)-2-naphthyl]oxymethyl]-1-piperidyl]ethyl]-4-piperidyl]-3-methyl-2-oxo-benzimidazol-1-yl]piperidine-2,6-dione (Example 137)

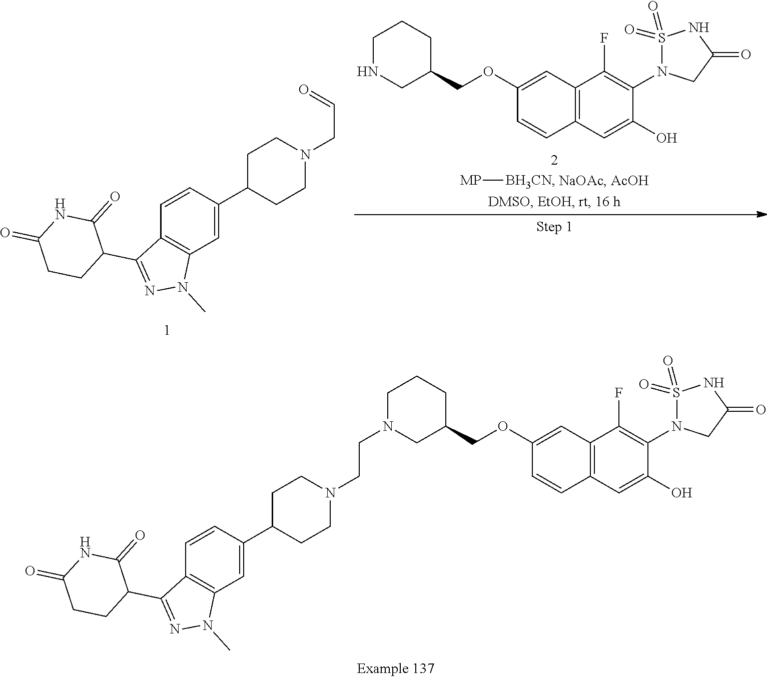

### Step 1: 3-[5-[1-[2-[(3R)-3-[[8-fluoro-6-hydroxy-7-(1,1,4-trioxo-1,2,5-thiadiazolidin-2-yl)-2-naphthyl]oxymethyl]-1-piperidyl]ethyl]-4-piperidyl]-3-methyl-2-oxo-benzimidazol-1-yl]piperidine-2,6-dione (Example 137)

Into a 50 mL single neck round bottom flask containing a well-stirred solution of 2-[4-[1-(2,6-dioxo-3-piperidyl)-3-methyl-2-oxo-benzimidazol-5-yl]-1-piperidyl]acetaldehyde (4, 100 mg, 150.47 μmol, TFA salt) and 5-[1-fluoro-3-hydroxy-7-[[(3R)-3-piperidyl]methoxy]-2-naphthyl]-1,1-dioxo-1,2,5-thiadiazolidin-3-one (5, 73.80 mg, 150.47 μmol) in anhydrous ethanol (5 mL) and DMSO (2 mL) were added acetic acid (315.00 mg, 5.25 mmol, 0.3 mL) and sodium acetate (86.40) mg, 1.05 mmol). After 1 h, MP-cyanoborohydride (250 mg, 0.5 mmol) was added. After 16 h, the reaction mixture was filtered through a sintered funnel, concentrated under reduced pressure and purified by reverse phase prep HPLC [Purification method: Column: X-Bridge C18 (150×19) 5 micron, mobile phase: 0.1% TFA in water; Mobile phase B: MeCN] to afford 3-[5-[1-[2-[(3R)-3-[[8-fluoro-6-hydroxy-7-(1,1,4-trioxo-1,2,5-thiadiazolidin-2-yl)-2-naphthyl]oxymethyl]-1-piperidyl]ethyl]-4-piperidyl]-3-methyl-2-oxo-benzimidazol-1-yl]piperidine-2,6-dione (Example 137, 40 mg, 44.28 μmol, 29% yield, TFA salt) as a colorless solid. LCMS (ES+) m/z 778.3 [M+H]⁺

¹H NMR (400 MHZ, DMSO-d₆) δ 11.10 (s, 1H), 9.76 (s, 1H), 7.69 (d, J=9.0 Hz, 1H), 7.27-7.21 (m, 1H), 7.18-7.12 (m, 1H), 7.09-7.02 (m, 3H), 6.93 (d, J=8.2 Hz, 1H), 5.34 (dd, J=12.9, 5.4 Hz, 1H), 4.16 (s, 2H), 4.11 (dd, J=10.0, 5.0 Hz, 1H), 4.04-3.96 (m, 1H), 3.70-3.60 (m, 4H), 3.33 (s, 3H), 3.17-3.06 (m, 2H), 2.98-2.83 (m, 3H), 2.77-2.58 (m, 2H), 2.54 (s, 1H), 2.40-2.27 (m, 1H), 2.12-1.83 (m, 7H), 1.82-1.67 (m, 1H), 1.48-1.33 (m, 1H).

### 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (Example 138)

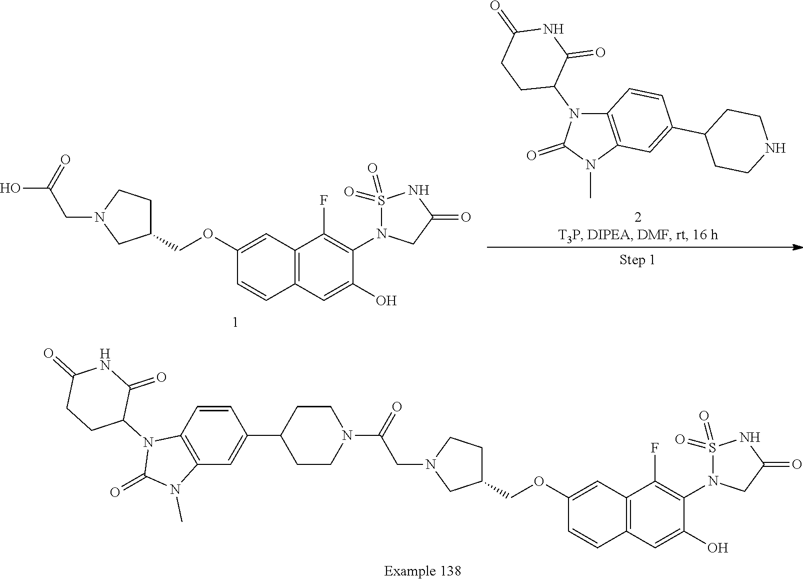 [SMILES: O=C(O)CN1CC[C@H](COc2ccc3cc(O)c(N4CC(=O)NS4(=O)=O)c(F)c3c2)C1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)CN4CC[C@H](COc5ccc6cc(O)c(N7CC(=O)NS7(=O)=O)c(F)c6c5)C4)CC3)cc21]

### Step 1: 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (Example 138)

Into a 50 mL single neck round bottom flask containing a well-stirred solution of 2-[(35)-3-[[8-fluoro-6-hydroxy-7-(1,1,4-trioxo-1,2,5-thiadiazolidin-2-yl)-2-naphthyl] oxymethyl]pyrrolidin-1-yl]acetic acid (1, 70 mg, 142.61 μmol, TFA salt) in anhydrous DMF (1.5 mL) was added propylphosphonic anhydride (50 wt. % in ethyl acetate) (136.13 mg, 213.92 μmol, 127.22 μL, 50% purity) followed by DIPEA (55.29 mg, 427.84 μmol, 74.52 μL). After 10 min, 3-[3-methyl-2-oxo-5-(4-piperidyl) benzimidazol-1-yl]piperidine-2,6-dione (2, 54.03 mg, 142.61 μmol, HCl salt) was added. After 16 h, the reaction mixture was concentrated under reduced pressure and the residue was purified by reverse phase prep HPLC [Purification method: X-select-C18 (150×19) mm, 5 micron column; Mobile phase A: 0.1% TFA in water; Mobile phase B: Acetonitrile] to obtain 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (Example 138, 52.02 mg, 40% yield, TFA salt) as an off-white solid. LCMS (ES+): m/z 778.0 [M+H]⁺

¹H NMR (400 MHZ, DMSO-d₆) δ 11.10 (s, 1H), 9.97 (s, 1H), 9.80 (s, 1H), 7.71 (d, J=9.0 Hz, 1H), 7.24 (s, 1H), 7.21-7.15 (m, 1H), 7.09-7.01 (m, 3H), 6.95-6.88 (m, 1H), 5.35 (dd, J=12.8, 5.3 Hz, 1H), 4.59-4.38 (m, 3H), 4.21 (d, J=2.9 Hz, 2H), 4.18-4.08 (m, 2H), 3.92-3.82 (m, 1H), 3.32 (d, J=5.6 Hz, 3H), 3.24-3.12 (m, 2H), 3.08-2.92 (m, 2H), 2.91-2.82 (m, 2H), 2.82-2.56 (m, 3H), 2.34-2.12 (m, 1H), 2.04-1.94 (m, 1H), 1.91-1.82 (m, 2H), 1.78-1.62 (m, 1H), 1.61-1.48 (m, 1H).

### 1-[4-[1-(2,6-dioxo-3-piperidyl)-3-methyl-2-oxo-benzimidazol-5-yl]phenyl]-3-[2-[[8-fluoro-6-hydroxy-7-(1,1,4-trioxo-1,2,5-thiadiazolidin-2-yl)-2-naphthyl]oxy]ethyl]urea (Example 139)

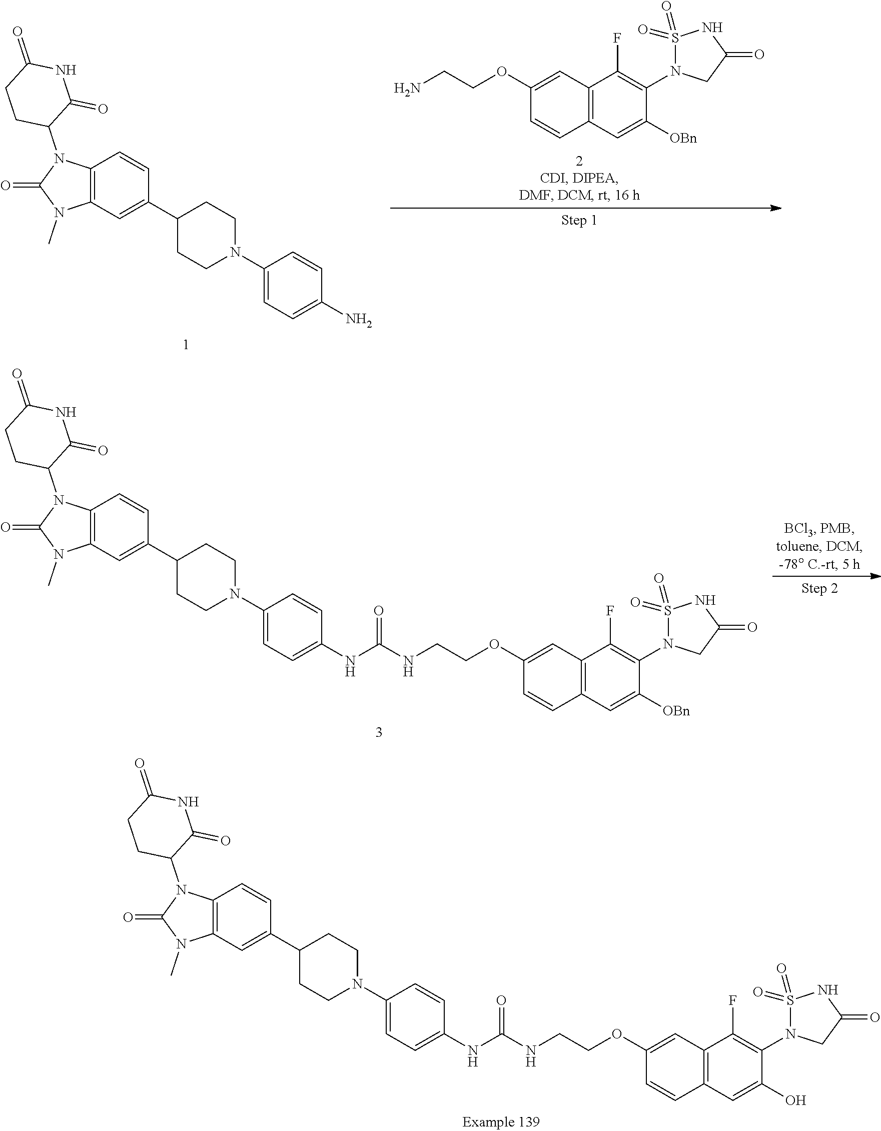 [SMILES: Cn1c(=O)n(C2CCC(=O)NC2=O)c2ccc(C3CCN(c4ccc(N)cc4)CC3)cc21 ; NCCOc1ccc2cc(OCc3ccccc3)c(N3CC(=O)NS3(=O)=O)c(F)c2c1 ; Cn1c(=O)n(C2CCC(=O)NC2=O)c2ccc(C3CCN(c4ccc(NC(=O)NCCOc5ccc6cc(OCc7ccccc7)c(N7CC(=O)NS7(=O)=O)c(F)c6c5)cc4)CC3)cc21 ; Cn1c(=O)n(C2CCC(=O)NC2=O)c2ccc(C3CCN(c4ccc(NC(=O)NCCOc5ccc6cc(O)c(N7CC(=O)NS7(=O)=O)c(F)c6c5)cc4)CC3)cc21]

### Step 1: 1-[2-[[6-benzyloxy-8-fluoro-7-(1,1,4-trioxo-1,2,5-thiadiazolidin-2-yl)-2-naphthyl]oxy]ethyl]-3-[4-[1-(2,6-dioxo-3-piperidyl)-3-methyl-2-oxo-benzimidazol-5-yl]phenyl]urea (3)

Into a 50 mL single neck round bottom flask containing a well-stirred solution of 3-[5-(4-aminophenyl)-3-methyl-2-oxo-benzimidazol-1-yl]piperidine-2,6-dione (1, 100 mg, 271.71 μmol) in anhydrous DMF (5 mL) were added DIPEA (175.58 mg, 1.36 mmol, 236.63 μL) and CDI (78.23 mg, 543.42 μmol). After 2 h, 5-[7-(2-aminoethoxy)-3-benzyloxy-1-fluoro-2-naphthyl]-1,1-dioxo-1,2,5-thiadiazolidin-3-one (2, 140.30 mg, 298.88 μmol, TFA salt) in DMF (0.5 mL) and DIPEA (0.1 mL) was added. After 16 h, the volatiles were removed under reduced pressure. The material was purified by reversed phase column chromatography [Purification method: Siliasep premium C18, 120 g column; Mobile phase A: 0.1% TFA in water; Mobile phase B: Acetonitrile] to afford 1-[2-[[6-benzyloxy-8-fluoro-7-(1,1,4-trioxo-1,2,5-thiadiazolidin-2-yl)-2-naphthyl]oxy]ethyl]-3-[4-[1-(2,6-dioxo-3-piperidyl)-3-methyl-2-oxo-benzimidazol-5-yl]phenyl]urea (3, 90 mg, 106.10 μmol, 39% yield) as an off-white solid. LCMS (ES+): m/z 822.1 [M+H]⁺

### Step 2: 1-[4-[1-(2,6-dioxo-3-piperidyl)-3-methyl-2-oxo-benzimidazol-5-yl]phenyl]-3-[2-[[8-fluoro-6-hydroxy-7-(1,1,4-trioxo-1,2,5-thiadiazolidin-2-yl)-2-naphthyl]oxy]ethyl]urea (Example 139)

Into a 50 mL single neck round bottom flask containing well-stirred solution of 1-[2-[[6-benzyloxy-8-fluoro-7-(1,1,4-trioxo-1,2,5-thiadiazolidin-2-yl)-2-naphthyl]oxy]ethyl]-3-[4-[1-(2,6-dioxo-3-piperidyl)-3-methyl-2-oxo-benzimidazol-5-yl]phenyl]urea (3, 120 mg, 141.46 μmol) in DCM (2 mL) and toluene (2 mL) was added pentamethylbenzene (104.86 mg, 707.30 μmol, 114.35 μL). Then boron trichloride (1.0 M in DCM) (331.50 mg, 2.83 mmol, 2.82 mL) was added at −78° C. The reaction mixture was stirred at RT for 5 h. The reaction mixture was cooled to −78° C. and quenched with 5% MeOH in DCM (2.5 mL). The reaction mixture was concentrated under reduced pressure, triturated with diethyl ether (10 mL) and filtered. The material was purified by reverse phase prep HPLC [Purification method: Column: X-Bridge C18 (19×150 mm), 5 mic; Mobile phase A: 0.1% TFA in water; Mobile phase B: MeCN] to afford 1-[4-[1-(2,6-dioxo-3-piperidyl)-3-methyl-2-oxo-benzimidazol-5-yl]phenyl]-3-[2-[[8-fluoro-6-hydroxy-7-(1,1,4-trioxo-1,2,5-thiadiazolidin-2-yl)-2-naphthyl]oxy]ethyl]urea (Example 139, 35 mg, 46.81 μmol, 33% yield) as an off-white solid. LCMS (ES+): m/z 732.0 [M+H]⁺

¹H NMR (400 MHZ, DMSO-d₆) δ 11.12 (s, 1H), 10.23 (s, 1H), 8.70 (s, 1H), 7.76-7.71 (m, 1H), 7.61-7.55 (m, 2H), 7.51-7.46 (m, 2H), 7.45 (d, J=1.7 Hz, 1H), 7.32-7.19 (m, 3H), 7.15 (d, J=8.3 Hz, 1H), 7.07 (s, 1H), 6.44 (t, J=5.4 Hz, 1H), 5.39 (dd, J=12.7, 5.4 Hz, 1H), 4.42 (s, 2H), 4.16 (t, J=5.5 Hz, 2H), 3.57-3.51 (m, 2H), 2.98-2.85 (m, 1H), 2.80-2.58 (m, 2H), 2.09-1.97 (m, 1H). Note: additional peaks under solvent signal

### 2-[4-[1-(2,6-dioxo-3-piperidyl)-3-methyl-2-oxo-benzimidazol-5-yl]-3,3-difluoro-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 140)

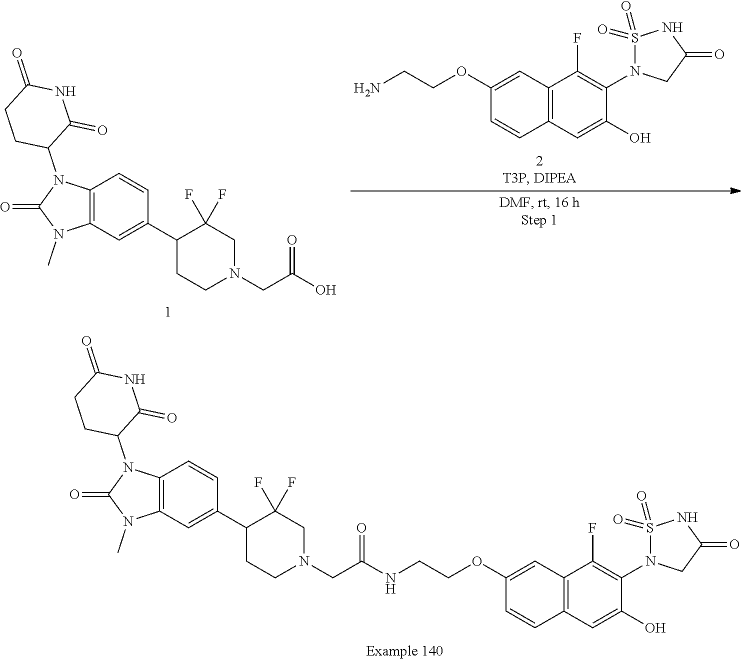 [SMILES: Cn1c(=O)n(C2CCC(=O)NC2=O)c2ccc(C3CCN(CC(=O)O)CC3(F)F)cc21 ; NCCOc1ccc2cc(O)c(N3CC(=O)NS3(=O)=O)c(F)c2c1 ; Cn1c(=O)n(C2CCC(=O)NC2=O)c2ccc(C3CCN(CC(=O)NCCOc4ccc5cc(O)c(N6CC(=O)NS6(=O)=O)c(F)c5c4)CC3(F)F)cc21]

### Step 1: 2-[4-[1-(2,6-dioxo-3-piperidyl)-3-methyl-2-oxo-benzimidazol-5-yl]-3,3-difluoro-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 140)

Into a 25 mL single neck, round bottom flask containing a well-stirred solution of 2-[4-[1-(2,6-dioxo-3-piperidyl)-3-methyl-2-oxo-benzimidazol-5-yl]-3,3-difluoro-1-piperidyl]acetic acid (1, 150 mg, 267.06 μmol, TFA salt) in anhydrous DMF (2 mL) were added DIPEA (172.58 mg, 1.34 mmol, 232.59 μL) and propylphosphonic anhydride (50 Wt % in EtOAc) (255.0 mg, 400.59 μmol, 255 μL) and 5-[7-(2-aminoethoxy)-1-fluoro-3-hydroxy-2-naphthyl]-1,1-dioxo-1,2,5-thiadiazolidin-3-one (2, 131.95 mg, 267.06 μmol, TFA salt). After 16 h, the reaction mixture was concentrated and purified by reverse-phase preparative HPLC (Column: X-select C18 (150×19) mm 5 micron; Mobile phase A: 0.1% Formic acid in water and Mobile phase B: CH₃CN) to afford 2-[4-[1-(2,6-dioxo-3-piperidyl)-3-methyl-2-oxo-benzimidazol-5-yl]-3,3-difluoro-1-piperidyl]-N-[2-[[8-fluoro-6-hydroxy-7-(1,1,4-trioxo-1,2,5-thiadiazolidin-2-yl)-2-naphthyl]oxy]ethyl]acetamide (Example 140, 39 mg, 46.36 μmol, 17% yield, formic acid salt) as a white powder. LCMS (ES+): m/z 774.0 [M+H]⁺

¹H NMR (400 MHZ, DMSO-d6): δ 11.12 (brs, 1H), 9.74 (brs, 1H), 8.46 (brs, 2H), 7.99 (t, J=5.60 Hz, 1H), 7.22 (s, 1H), 7.15 (d, J=6.40 Hz, 1H), 7.09-7.06 (m, 2H), 7.02 (s, 1H), 6.96 (d, J=8.40 Hz, 1H), 5.31 (dd, J=4.80, 12.60 Hz, 1H), 4.15 (t, J=5.60 Hz, 1H), 4.09 (s, 2H), 3.33 (s, 3H), 3.21-2.91 (m, 5H), 2.89-2.85 (m, 2H), 2.71-2.60 (m, 3H), 2.40 (t, J=11.20 Hz, 1H), 2.34-2.23 (m, 1H), 2.04-2.01 (m, 1H), 1.80-1.69 (m, 1H).

### 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (Example 141)

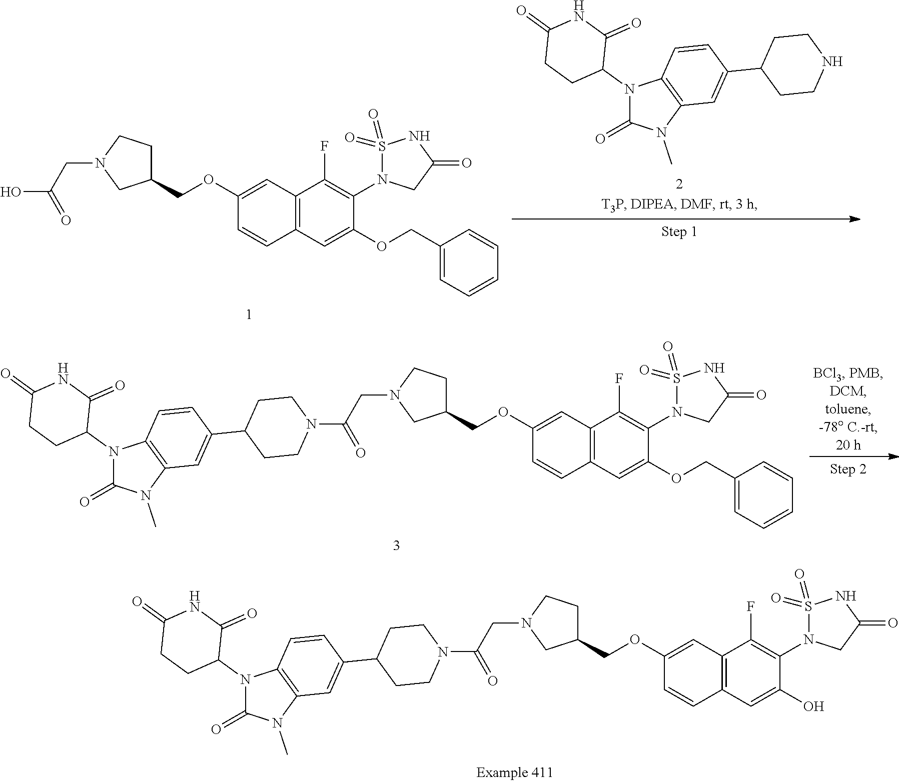 [SMILES: O=C(O)CN1CC[C@@H](COc2ccc3cc(OCc4ccccc4)c(N4CC(=O)NS4(=O)=O)c(F)c3c2)C1 ; Cn1c(=O)n(C2CCC(=O)NC2=O)c2ccc(C3CCNCC3)cc21 ; Cn1c(=O)n(C2CCC(=O)NC2=O)c2ccc(C3CCN(C(=O)CN4CC[C@@H](COc5ccc6cc(OCc7ccccc7)c(N7CC(=O)NS7(=O)=O)c(F)c6c5)C4)CC3)cc21 ; Cn1c(=O)n(C2CCC(=O)NC2=O)c2ccc(C3CCN(C(=O)CN4CC[C@@H](COc5ccc6cc(O)c(N7CC(=O)NS7(=O)=O)c(F)c6c5)C4)CC3)cc21]

### Step 1: 3-[5-[1-[2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (3)

Into a 20 mL capped vial containing a well-stirred solution of 2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetic acid (1, 280 mg, 398.55 μmol, TFA salt) in DMF (2.00 mL) was added DIPEA (515.10 mg, 3.99 mmol, 694.20 μL) and 1-propanephosphonic anhydride (50% in Ethyl Acetate) (507.24 mg, 797.10 μmol, 50% purity). After 5 min, 3-[3-methyl-2-oxo-5-(4-piperidyl)benzimidazol-1-yl]piperidine-2,6-dione (2, 136.46 mg, 360.19 μmol, HCl salt) was added. After 3 h, the reaction mixture was concentrated under reduced pressure and purified by reverse phase preparative HPLC [Purification method: X-Select C18 (19 mm×150 mm) 5 micron; Mobile phase A: 0.1% TFA in H₂O: Mobile phase B: MeCN] to afford 3-[5-[1-[2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (3, 85 mg, 82.23 μmol, 21% yield, TFA salt) as a colorless solid. LCMS (ES+): m/z 868.2 [M+H]⁺

### Step 2: 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (Example 141)

Into a 25 mL single neck round bottom flask containing a well-stirred solution of 3-[5-[1-[2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (3, 85 mg, 82.23 μmol, TFA salt) in a mixture of anhydrous DCM (2 mL) and toluene (2 mL) was added pentamethylbenzene (73.14 mg, 493.40 μmol, 79.76 μL) and the reaction mixture was cooled to −78° C. Boron trichloride (1.0 M in DCM) (2.56 mmol, 2.5 mL) was added dropwise over a period of 3 min. The reaction mixture was stirred at RT for 20 h. The reaction mixture was cooled to −78° C. and quenched slowly with 10% MeOH in DCM (4 mL). The reaction mixture was concentrated under reduced pressure at 30° C. The residue was triturated with Et₂O (20 mL), filtered and purified by reverse phase preparative HPLC [Purification method: X-Select C18 (19 mm×150 mm) 5 micron; Mobile phase A: 0.1% TFA in H₂O and Mobile phase B: MeCN] to afford 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]acetyl]-4-piperidyl]-3-methyl-2-oxo-benzimidazol-1-yl]piperidine-2,6-dione (Example 141, 43 mg, 47.63 μmol, 58% yield, TFA salt) as a colorless solid. LCMS (ES+): m/z 778.0 [M+H]⁺

¹H NMR (400 MHZ, DMSO-d₆) δ 11.09 (s, 1H), 9.97 (s, 1H), 9.68 (s, 1H), 7.71 (d, J=9.1 Hz, 1H), 7.26-7.22 (m, 1H), 7.19-7.14 (m, 1H), 7.09-7.01 (m, 3H), 6.95-6.89 (m, 1H), 5.34 (dd, J=12.8, 5.4 Hz, 1H), 4.59-4.41 (m, 3H), 4.24-4.08 (m, 4H), 3.79-3.64 (m, 2H), 3.22-3.12 (m, 2H), 3.10-2.93 (m, 2H), 2.92-2.82 (m, 2H), 2.81-2.69 (m, 1H), 2.65-2.57 (m, 1H), 2.23-2.12 (m, 1H), 2.05-1.95 (m, 2H), 1.90-1.81 (m, 2H), 1.77-1.63 (m, 1H), 1.62-1.47 (m, 1H). Note: additional peaks under solvent signal

### 3-[5-[4-[2-[(3R)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-1-piperidyl]-3-methyl-2-oxo-benzimidazol-1-yl]piperidine-2,6-dione (Example 142)

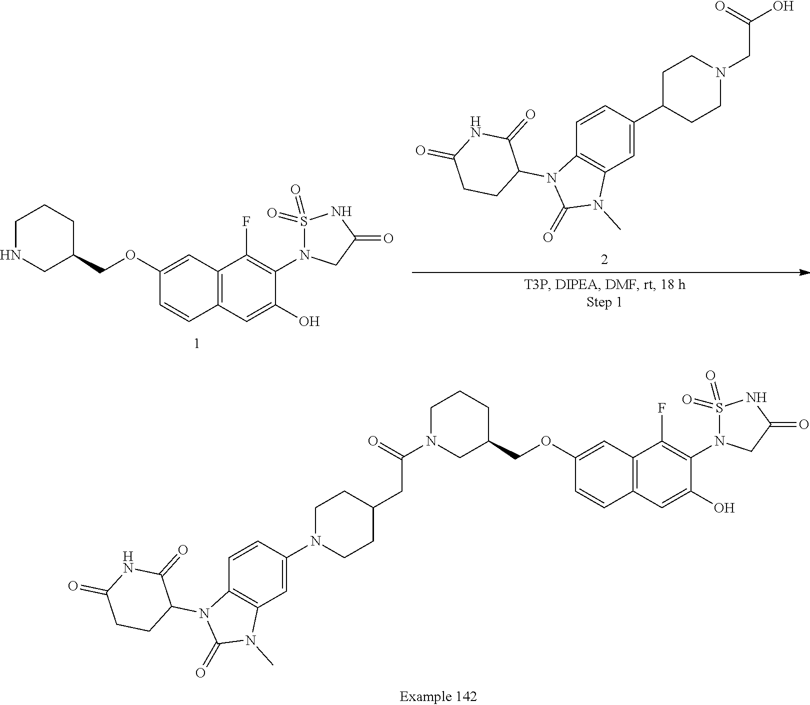 [SMILES: O=C1CN(c2c(O)cc3ccc(OC[C@@H]4CCCNC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(N3CCC(CC(=O)N4CCC[C@@H](COc5ccc6cc(O)c(N7CC(=O)NS7(=O)=O)c(F)c6c5)C4)CC3)cc21]

### Step 1: 3-[5-[4-[2-[(3R)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-1-piperidyl]-3-methyl-2-oxo-benzimidazol-1-yl]piperidine-2,6-dione (Example 142)

Into a 25 mL single neck round bottom flask containing a well-stirred solution of 2-[1-[1-(2,6-dioxo-3-piperidyl)-3-methyl-2-oxo-benzimidazol-5-yl]-4-piperidyl]acetic acid (2, 130 mg, 256.48 μmol) in anhydrous DMF (3 mL) were added DIPEA (99.44 mg, 769.43 μmol, 134.02 μL) and propylphosphonic anhydride solution (≥50 wt. % in ethyl acetate) (244.82 mg, 384.71 μmol, 0.25 mL, 50% purity). After 5 min, 5-[1-fluoro-3-hydroxy-7-[[(3R)-3-piperidyl]methoxy]-2-naphthyl]-1,1-dioxo-1,2,5-thiadiazolidin-3-one (1, 117.87 mg, 282.12 μmol) was added. After 18 h, the volatiles were removed under reduced pressure and the residue was purified by reverse phase prep HPLC [Purification method: Column: X-Select C18 (150×19) 5 micron; Mobile phase A: 0.1% TFA in water; Mobile phase B: MeCN] to afford 3-[5-[4-[2-[(3R)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-1-piperidyl]-3-methyl-2-oxo-benzimidazol-1-yl]piperidine-2,6-dione (Example 142, 34 mg, 35.33 μmol, 14% yield, TFA salt) as an off white solid. LCMS (ES+): m/z 792.0 [M+H]⁺

¹H NMR (400 MHZ, DMSO-d₆) δ 11.13 (s, 1H), 9.89 (s, 1H), 7.70 (dd, J=9.2, 4.1 Hz, 1H), 7.49 (s, 1H), 7.27-7.12 (m, 4H), 7.05 (s, 1H), 5.39 (dd, J=12.7, 5.4 Hz, 1H), 4.26 (d, J=5.2 Hz, 2H), 4.14-3.88 (m, 4H), 3.84-3.78 (m, 1H), 3.17-3.05 (m, 2H), 2.95-2.83 (m, 2H), 2.78-2.58 (m, 3H), 2.44-2.35 (m, 2H), 2.14-1.85 (m, 6H), 1.81-1.29 (m, 5H). Note: additional peaks under solvent signal

### 3-[5-[4-[2-[(3S)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-1-piperidyl]-3-methyl-2-oxo-benzimidazol-1-yl]piperidine-2,6-dione (Example 143)

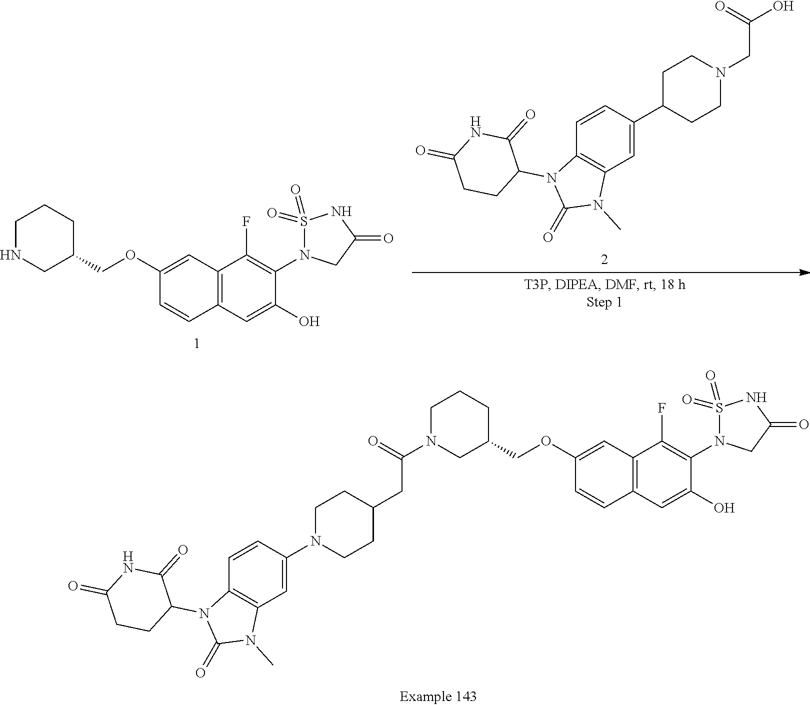 [SMILES: O=C1CN(c2c(O)cc3ccc(OC[C@H]4CCCNC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(N3CCC(CC(=O)N4CCC[C@H](COc5ccc6cc(O)c(N7CC(=O)NS7(=O)=O)c(F)c6c5)C4)CC3)cc21]

3-[5-[4-[2-[(3S)-3-[[8-fluoro-6-hydroxy-7-(1,1,4-trioxo-1,2,5-thiadiazolidin-2-yl)-2-naphthyl]oxymethyl]-1-piperidyl]-2-oxo-ethyl]-1-piperidyl]-3-methyl-2-oxo-benzimidazol-1-yl]piperidine-2,6-dione (Example 143, 60.0 mg, 62.49 μmol, 18% yield, TFA salt) was prepared from 5-[1-fluoro-3-hydroxy-7-[[(3S)-3-piperidyl]methoxy]-2-naphthyl]-1,1-dioxo-1,2,5-thiadiazolidin-3-one (1) and 2-[[1-[1-(2,6-dioxo-3-piperidyl)-3-methyl-2-oxo-benzimidazol-5-yl]-4-piperidyl]acetic acid (2) using the same procedure as Example 142. LCMS (ES+): m/z 792.0 [M+H]⁺

¹H NMR (400 MHZ, DMSO-d₆) δ 11.14 (s, 1H), 9.83 (s, 1H), 7.75-7.66 (m, 1H), 7.53-7.41 (m, 1H), 7.26-7.15 (m, 3H), 7.05 (s, 1H), 5.40 (dd, J=12.8, 5.4 Hz, 1H), 4.26-4.22 (m, 2H), 4.14-3.88 (m, 4H), 3.37 (d, J=2.6 Hz, 3H), 3.17-3.06 (m, 1H), 2.95-2.83 (m, 2H), 2.78-2.63 (m, 3H), 2.42-2.36 (m, 2H), 2.14-1.84 (m, 6H), 1.80-1.66 (m, 1H), 1.64-1.29 (m, 5H).

### 3-[5-[1-[3-[(3R)-3-[[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 144)

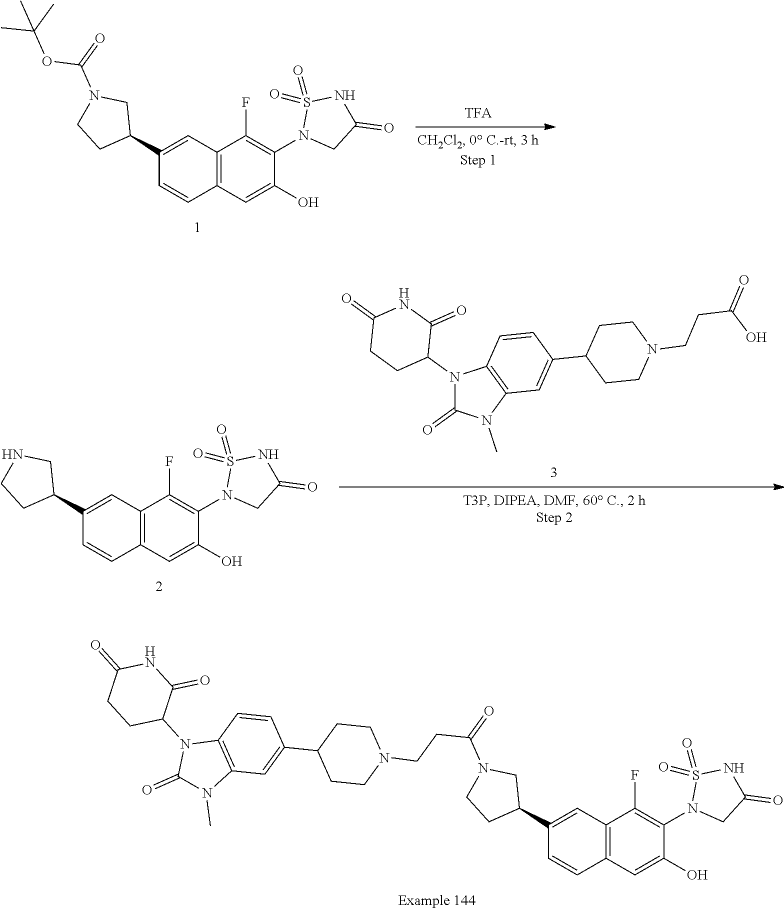 [SMILES: CC(C)(C)OC(=O)N1CC[C@H](c2ccc3cc(O)c(N4CC(=O)NS4(=O)=O)c(F)c3c2)C1 ; O=C1CN(c2c(O)cc3ccc([C@H]4CCNC4)cc3c2F)S(=O)(=O)N1 ; Cn1c(=O)n(C2CCC(=O)NC2=O)c2ccc(C3CCN(CCC(=O)O)CC3)cc21 ; Cn1c(=O)n(C2CCC(=O)NC2=O)c2ccc(C3CCN(CCC(=O)N4CC[C@H](c5ccc6cc(O)c(N7CC(=O)NS7(=O)=O)c(F)c6c5)C4)CC3)cc21]

### Step 1: 5-[1-fluoro-3-hydroxy-7-[(3R)-pyrrolidin-3-yl]-2-naphthyl]-1,1-dioxo-1,2,5-thiadiazolidin-3-one (2)

In to a 25 mL round-neck, round bottom flask containing a well-stirred solution of tert-butyl (3R)-3-[8-fluoro-6-hydroxy-7-(1,1,4-trioxo-1,2,5-thiadiazolidin-2-yl)-2-naphthyl]pyrrolidine-1-carboxylate (1, 250 mg, 537.06 μmol) in anhydrous DCM (3 mL) was added TFA (1.53 g, 13.43 mmol, 1.03 mL) at 0° C. under nitrogen atmosphere. The resulting mixture was stirred at rt for 3 h. The solvent was evaporated and the residue triturated with Et₂O to afford 5-[1-fluoro-3-hydroxy-7-[(3R)-pyrrolidin-3-yl]-2-naphthyl]-1,1-dioxo-1,2,5-thiadiazolidin-3-one (2, 250 mg, 479.76 μmol, 89% yield, TFA sat) as an off-white solid. LCMS (ES+): m/z 366.0 [M+H]⁺

### Step 2: 3-[5-[1-[3-[(3R)-3-[[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 144)

Into a 25 mL single neck round bottom flask containing a well-stirred solution of 3-[4-[1-(2,6-dioxo-3-piperidyl)-3-methyl-2-oxo-benzimidazol-5-yl]cyclohexyl]propanoic acid (3, 240.33 mg, 379.97 μmol, TFA salt) and 5-[1-fluoro-3-hydroxy-7-[(3R)-pyrrolidin-3-yl]-2-naphthyl]-1,1-dioxo-1,2,5-thiadiazolidin-3-one (2, 180 mg, 345.43 μmol, TFA salt) in dry DMF (4 mL) were added 1-propanephosphonic anhydride (50 Wt % in EtOAc) (263.78 mg, 414.52 μmol) and DIPEA (133.93 mg, 1.04 mmol, 180.50 μL). The reaction was stirred at 60° C. for 2 h. The solvent was removed and the residue purified by preparative-HPLC [Column: X-SELECT-C18 (150×30) mm, 5 micron; Mobile Phase A: 0.1% TFA in water, Mobile Phase B: CH₃CN) to afford 3-[5-[1-[3-[(3R)-3-[[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 144, 34 mg, 37.54 μmol, 11% yield, TFA salt) as a brick red solid. LCMS (ES+): m/z 762.0 [M+H]⁺

¹H NMR (400 MHz, DMSO-d₆) δ 11.10 (s, 1H), 9.78 (d, J=2.2 Hz, 1H), 8.99 (s, 1H), 7.79-7.69 (m, 2H), 7.50-7.43 (m, 1H), 7.09-7.02 (m, 3H), 6.95-6.88 (m, 1H), 5.35 (dd, J=12.6, 5.4 Hz, 1H), 4.09 (s, 2H), 3.98 (dt, J=35.3, 9.0 Hz, 1H), 3.74-3.48 (m, 6H), 3.16-3.02 (m, 2H), 2.95-2.80 (m, 4H), 2.72-2.57 (m, 2H), 2.23-2.10 (m, 1H), 2.06-1.87 (m, 5H). Note: additional peaks under solvent signal

### 3-[5-[1-[3-[(3S)-3-[[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 145)

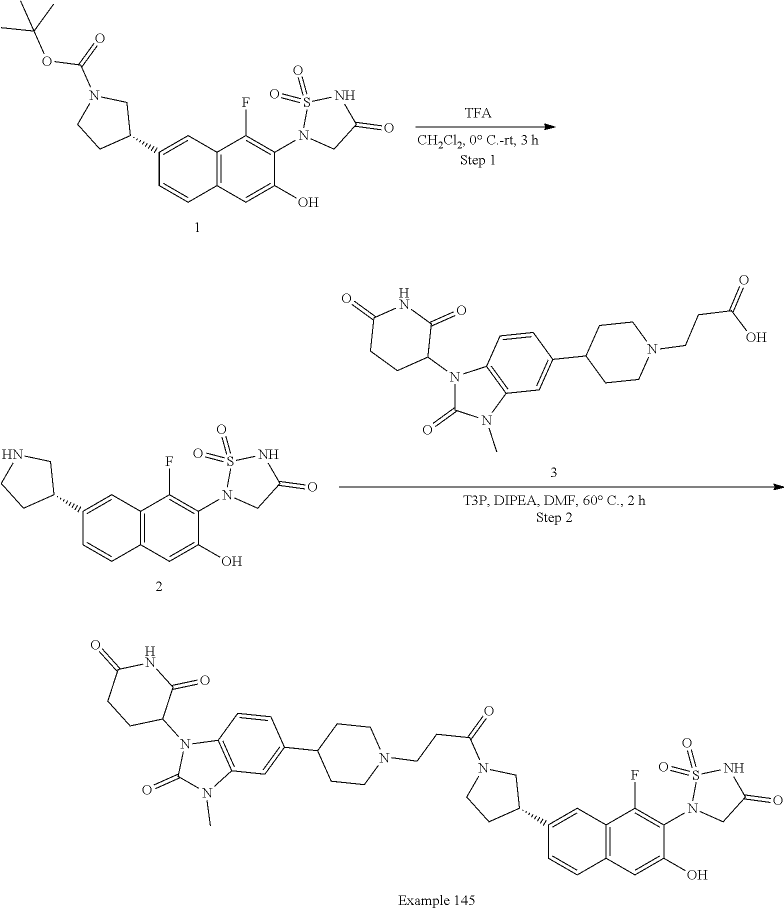 [SMILES: CC(C)(C)OC(=O)N1CC[C@@H](c2ccc3cc(O)c(N4CC(=O)NS4(=O)=O)c(F)c3c2)C1 ; O=C1CN(c2c(O)cc3ccc([C@@H]4CCNC4)cc3c2F)S(=O)(=O)N1 ; Cn1c(=O)n(C2CCC(=O)NC2=O)c2ccc(C3CCN(CCC(=O)O)CC3)cc21 ; Cn1c(=O)n(C2CCC(=O)NC2=O)c2ccc(C3CCN(CCC(=O)N4CC[C@@H](c5ccc6cc(O)c(N7CC(=O)NS7(=O)=O)c(F)c6c5)C4)CC3)cc21]

### Step 1: 5-[1-fluoro-3-hydroxy-7-[(3S)-pyrrolidin-3-yl]-2-naphthyl]-1,1-dioxo-1,2,5-thiadiazolidin-3-one (2)

Into a 25 mL single neck round bottom flask containing a well-stirred solution of tert-butyl (3S)-3-[8-fluoro-6-hydroxy-7-(1,1,4-trioxo-1,2,5-thiadiazolidin-2-yl)-2-naphthyl]pyrrolidine-1-carboxylate (1, 200 mg, 423.98 μmol, second eluted isomer) in dry DCM (3 mL) was added TFA (740.00 mg, 6.49 mmol, 0.5 mL) under nitrogen atmosphere at 0° C. The reaction mixture was stirred for 3 h at ambient temperature. The reaction mixture was concentrated under reduced pressure and co-distilled with toluene (2×10 mL) and triturated with diethyl ether (10 mL) to afford 5-[1-fluoro-3-hydroxy-7-[(3S)-pyrrolidin-3-yl]-2-naphthyl]-1,1-dioxo-1,2,5-thiadiazolidin-3-one (2, 190 mg, 357.45 μmol, 84% yield, TFA salt) as a brown solid. LCMS (ES+): m/z 366.2 [M+H]⁺

### Step 2: 3-[5-[1-[3-[(3S)-3-[[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 145)

Into a 25 mL single neck round bottom flask containing a well-stirred solution of 3-[4-[1-(2,6-dioxo-3-piperidyl)-3-methyl-2-oxo-benzimidazol-5-yl]-1-piperidyl]propanoic acid (3, 248.69 mg, 393.19 μmol, TFA salt) in dry DMF (4 mL) were added 5-[1-fluoro-3-hydroxy-7-[(3S)-pyrrolidin-3-yl]-2-naphthyl]-1,1-dioxo-1,2,5-thiadiazolidin-3-one (2, 190 mg, 357.45 μmol, TFA salt), 1-propanephosphonic anhydride (50 Wt % in EtOAc) (341.20 mg, 536.17 μmol, 50% purity) and DIPEA (138.59 mg, 1.07 mmol, 186.78 μL). The reaction was stirred at 60° C. for 2 h. The volatiles were removed under reduced pressure and diluted with water (10 mL), and the precipitate purified by preparative-HPLC [Column: X-SELECT-C18 (250×19) mm, 5 microns; Mobile phase A: 0.1% TFA in water and Mobile phase B: CH₃CN] to afford 3-[5-[1-[3-[(3S)-3-[[8-fluoro-6-hydroxy-7-(1,1,4-trioxo-1,2,5-thiadiazolidin-2-yl)-2-naphthyl]pyrrolidin-1-yl]-3-oxo-propyl]-4-piperidyl]-3-methyl-2-oxo-benzimidazol-1-yl]piperidine-2,6-dione (Example 145, 46 mg, 51.33 μmol, 14% yield, TFA salt) as a pale yellow solid. LCMS (ES+): m/z 762.0 [M+H]⁺

¹H NMR (400 MHZ, DMSO-d₆) δ 11.10 (s, 1H), 10.13 (s, 1H), 9.10 (s, 1H), 7.80-7.71 (m, 2H), 7.53-7.45 (m, 1H), 7.09-7.03 (m, 3H), 6.95-6.88 (m, 1H), 5.40-5.30 (m, 1H), 4.25 (d, J=2.9 Hz, 2H), 4.02 (dd, J=9.6, 7.3 Hz, 1H), 3.94 (dd, J=11.4, 7.5 Hz, 1H), 3.76-3.55 (m, 5H), 3.41-3.38 (m, 2H), 3.34 (d, J=2.3 Hz, 3H), 3.16-3.04 (m, 2H), 2.97-2.78 (m, 4H), 2.77-2.58 (m, 2H), 2.45-2.28 (m, 1H), 2.21-2.08 (m, 1H), 2.06-1.85 (m, 5H).

### 3-[5-[1-[[5-[5-fluoro-7-hydroxy-6-(1,1,4-trioxo-1,2,5-thiadiazolidin-2-yl)-2-naphthyl]-1,3,4-oxadiazol-2-yl]methyl]-4-piperidyl]-3-methyl-2-oxo-benzimidazol-1-yl]piperidine-2,6-dione (Example 146)

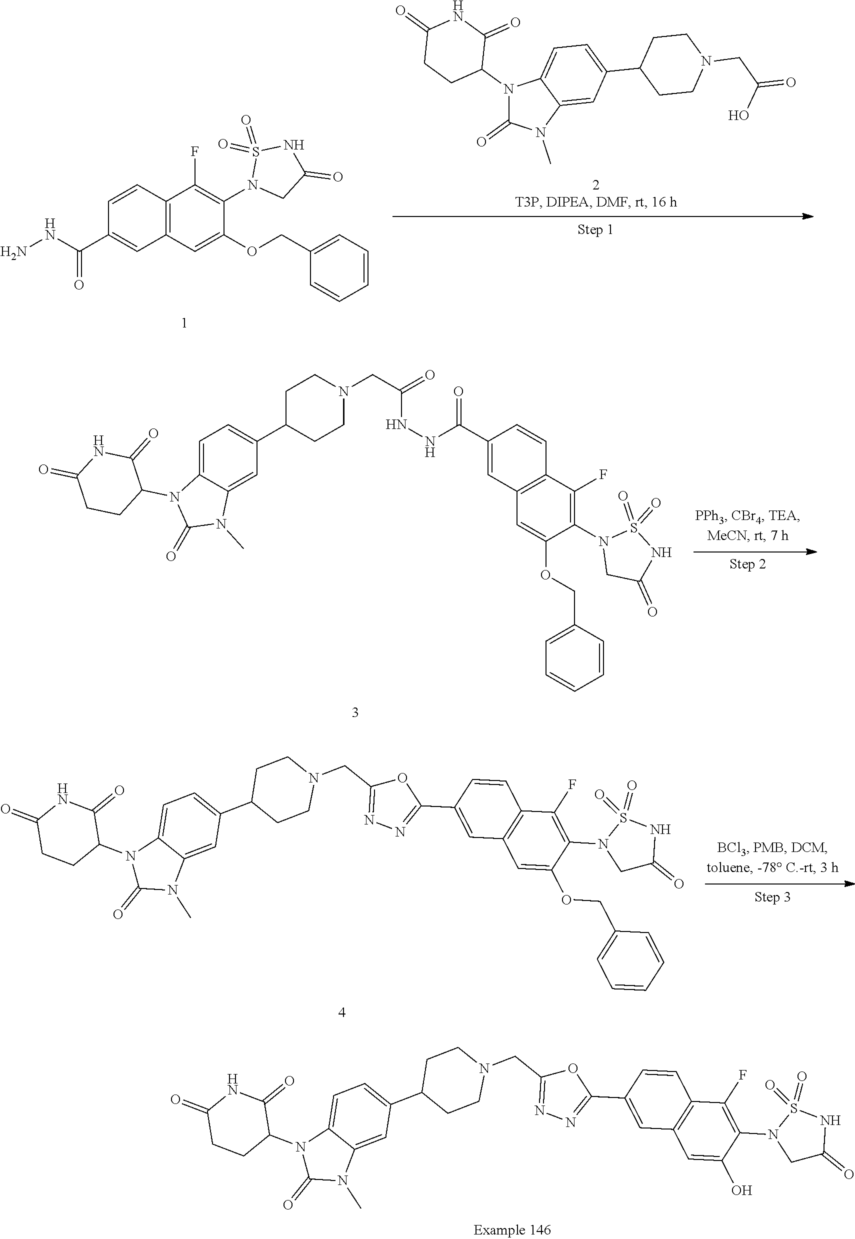 [SMILES: NNC(=O)c1ccc2c(F)c(N3CC(=O)NS3(=O)=O)c(OCc3ccccc3)cc2c1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)NNC(=O)c4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN(Cc4nnc(-c5ccc6c(F)c(N7CC(=O)NS7(=O)=O)c(OCc7ccccc7)cc6c5)o4)CC3)cc21 ; Cn1c(=O)n(C2CCC(=O)NC2=O)c2ccc(C3CCN(Cc4nnc(-c5ccc6c(F)c(N7CC(=O)NS7(=O)=O)c(O)cc6c5)o4)CC3)cc21]

### Step 1: 7-benzyloxy-N′-[2-[4-[1-(2,6-dioxo-3-piperidyl)-3-methyl-2-oxo-benzimidazol-5-yl]-1-piperidyl]acetyl]-5-fluoro-6-(1,1,4-trioxo-1,2,5-thiadiazolidin-2-yl)naphthalene-2-carbohydrazide (3)

Into a 20 mL vial containing a well-stirred solution of 7-benzyloxy-5-fluoro-6-(1,1,4-trioxo-1,2,5-thiadiazolidin-2-yl)naphthalene-2-carbohydrazide (1, 300.00 mg, 436.68 μmol, HCl salt) and 2-[4-[1-(2,6-dioxo-3-piperidyl)-3-methyl-2-oxo-benzimidazol-5-yl]-1-piperidyl]acetic acid (2, 227.87 mg, 436.68 μmol, TFA salt) in DMF (8 mL) was added DIPEA (282.19 mg, 2.18 mmol) followed by of 1-propanephosphonic anhydride (50% in ethyl acetate) (416.83 mg, 655.03 μmol, 50% purity). After 16 h, the reaction mixture was concentrated under reduced pressure and water (5 mL) was added. The precipitate was filtered and dried under vacuum to afford 7-benzyloxy-N′-[2-[4-[1-(2,6-dioxo-3-piperidyl)-3-methyl-2-oxo-benzimidazol-5-yl]-1-piperidyl]acetyl]-5-fluoro-6-(1,1,4-trioxo-1,2,5-thiadiazolidin-2-yl)naphthalene-2-carbohydrazide (3, 0.31 g, 350.55 μmol, 80% yield) as an off-white solid. The material was used in the next step without further purification. LCMS (ES+): m/z 827.0 [M+H]⁺

### Step 2: 3-[5-[1-[[5-[7-benzyloxy-5-fluoro-6-(1,1,4-trioxo-1,2,5-thiadiazolidin-2-yl)-2-naphthyl]-1,3,4-oxadiazol-2-yl]methyl]-4-piperidyl]-3-methyl-2-oxo-benzimidazol-1-yl]piperidine-2,6-dione (4)

Into a 20 mL vial containing a well-stirred solution of 7-benzyloxy-N′-[2-[4-[1-(2,6-dioxo-3-piperidyl)-3-methyl-2-oxo-benzimidazol-5-yl]-1-piperidyl]acetyl]-5-fluoro-6-(1,1,4-trioxo-1,2,5-thiadiazolidin-2-yl)naphthalene-2-carbohydrazide (3, 0.37 g, 418.40 μmol) in MeCN (8 mL) was added triphenylphosphine (219.48 mg, 836.79 μmol) and triethylamine (84.68 mg, 836.79 μmol) followed by carbon tetrabromide (277.51 mg, 836.79 μmol). After 7 h, the reaction mixture was concentrated under reduced pressure and the residue was purified by reverse phase column chromatography [Purification method: Biotage 120 g. C-18 column; Mobile phase A: 0.1% TFA in water; Mobile phase B: Acetonitrile] to obtain 3-[5-[1-[[5-[7-benzyloxy-5-fluoro-6-(1,1,4-trioxo-1,2,5-thiadiazolidin-2-yl)-2-naphthyl]-1,3,4-oxadiazol-2-yl]methyl]-4-piperidyl]-3-methyl-2-oxo-benzimidazol-1-yl]piperidine-2,6-dione (4, 0.13 g, 116.33 μmol, 28% yield, 72% purity. TFA salt) as a light brown solid. LCMS (ES+): m/z 808.9 [M+H]⁺

### Step 3: 3-[5-[1-[[5-[5-fluoro-7-hydroxy-6-(1,1,4-trioxo-1,2,5-thiadiazolidin-2-yl)-2-naphthyl]-1,3,4-oxadiazol-2-yl]methyl]-4-piperidyl]-3-methyl-2-oxo-benzimidazol-1-yl]piperidine-2,6-dione (Example 146)

Into a 25 mL round bottom flask containing a well-stirred solution of 3-[5-[1-[[5-[7-benzyloxy-5-fluoro-6-(1,1,4-trioxo-1,2,5-thiadiazolidin-2-yl)-2-naphthyl]-1,3,4-oxadiazol-2-yl]methyl]-4-piperidyl]-3-methyl-2-oxo-benzimidazol-1-yl]piperidine-2,6-dione (4, 0.095 g, 91.13 μmol, TFA salt) and pentamethylbenzene (67.55 mg, 455.63 μmol) in a mixture of DCM (0.4 mL) and toluene (0.4 mL) was added boron trichloride (1.0 M in DCM) (320.32 mg, 2.73 mmol, 1.23 mL) at −78° C. under nitrogen atmosphere. The reaction mixture was stirred at ambient temperature for 3 h. The reaction was cooled to −78° C. and quenched via dropwise addition of 10% MeOH in DCM (1.2 mL). The volatiles were removed under reduced pressure and the residue was purified by reverse phase prep HPLC [Purification method: X-Bridge C18 (19×150 mm) 5.0μ; Mobile phase A: 0.1% TFA in water; Mobile phase B: Acetonitrile) to afford 3-[5-[1-[[5-[5-fluoro-7-hydroxy-6-(1,1,4-trioxo-1,2,5-thiadiazolidin-2-yl)-2-naphthyl]-1,3,4-oxadiazol-2-yl]methyl]-4-piperidyl]-3-methyl-2-oxo-benzimidazol-1-yl]piperidine-2,6-dione (Example 146, 10 mg, 11.89 μmol, 13% yield, TFA salt) as an off-white solid. LCMS (ES+): m/z 719.0 [M+H]⁺

¹H NMR (400 MHZ, DMSO-d6): δ 11.10 (s, 1H), 10.33 (s, 1H), 8.50 (s, 1H), 8.14 (d, J=11.20 Hz, 1H), 7.98-7.94 (m, 1H), 7.33 (s, 1H), 7.10-7.04 (m, 2H), 6.92 (d, J=10.40 Hz, 1H), 5.38-5.32 (m, 1H), 4.80 (bs, 2H), 4.17 (s, 2H), 3.66-3.61 (m, 2H), 3.34 (s, 3H), 3.05-2.85 (m, 3H), 2.73-2.57 (m, 2H), 2.10-1.98 (m, 5H).

### 3-[5-[1-[2-[(3S)-3-[[8-fluoro-6-hydroxy-7-(1,1,4-trioxo-1,2,5-thiadiazolidin-2-yl)-2-naphthyl]oxymethyl]-1-piperidyl]ethyl]-4-piperidyl]-3-methyl-2-oxo-benzimidazol-1-yl]piperidine-2,6-dione (Example 147)

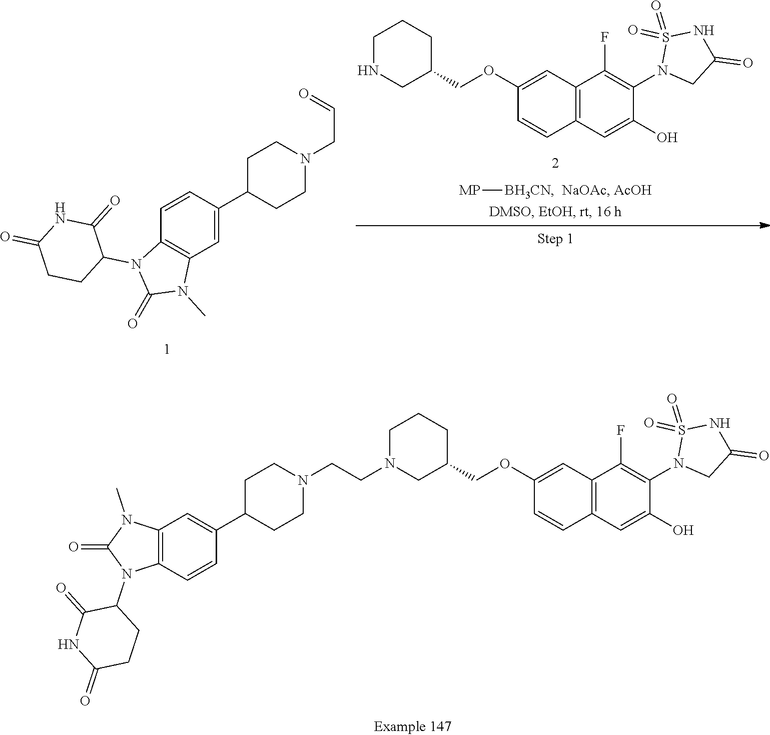

### Step 1: 3-[5-[1-[2-[(3S)-3-[[8-fluoro-6-hydroxy-7-(1,1,4-trioxo-1,2,5-thiadiazolidin-2-yl)-2-naphthyl]oxymethyl]-1-piperidyl]ethyl]-4-piperidyl]-3-methyl-2-oxo-benzimidazol-1-yl]piperidine-2,6-dione (Example 147)

Into a 50 mL single neck round bottom flask containing well-stirred solution of 2-[4-[1-(2,6-dioxo-3-piperidyl)-3-methyl-2-oxo-benzimidazol-5-yl]-1-piperidyl]acetaldehyde (1, 241.91 mg, 446.78 μmol) and (S)-5-(1-fluoro-3-hydroxy-7-(piperidin-3-ylmethoxy)naphthalen-2-yl)-1,2,5-thiadiazolidin-3-one 1,1-dioxide (2, 150 mg, 297.85 μmol) in ethanol (4 mL) and DMSO (1.5 mL) were added sodium acetate (61.08 mg, 744.63 μmol) and acetic acid (178.86 mg, 2.98 mmol, 170.34 μL) at RT. The resulting suspension was stirred for 1 h. Then, MP-cyanoborohydride 2.0 mmol/g (250) mg, 744.63 μmol) was added and stirring continued for 16 h at RT. The reaction mixture was filtered through a sintered funnel, filtrate was concentrated under reduced pressure to afford the crude compound, which was purified by reverse phase prep HPLC [Purification method: Column: X Select C18 (250×19) mm, 5 micron; Mobile phase A: 0.1% TFA in water; Mobile phase B: MeCN] to afford the 3-[5-[1-[2-[(3S)-3-[[8-fluoro-6-hydroxy-7-(1,1,4-trioxo-1,2,5-thiadiazolidin-2-yl)-2-naphthyl]oxymethyl]-1-piperidyl]ethyl]-4-piperidyl]-3-methyl-2-oxo-benzimidazol-1-yl]piperidine-2,6-dione (Example 147, 22 mg, 21.69 μmol, 7.28% yield, TFA salt) as an off-white solid. LCMS (ES+): m/z 778.2 [M+H]⁺

¹H NMR (400 MHZ, DMSO-d₆) δ 11.11 (s, 1H), 9.69 (s, 1H), 7.70 (d, J=9.0 Hz, 1H), 7.26-7.22 (m, 1H), 7.15 (dd, J=9.0, 2.5 Hz, 1H), 7.11-7.03 (m, 3H), 6.92 (d, J=8.1 Hz, 1H), 5.35 (dd, J=12.8, 5.4 Hz, 1H), 4.14 (s, 2H), 4.12-4.09 (m, 1H), 4.02 (t, J=8.6 Hz, 1H), 3.64-3.55 (m, 3H), 3.34 (s, 3H), 3.19-3.01 (m, 2H), 2.96-2.78 (m, 4H), 2.76-2.58 (m, 3H), 2.13-1.83 (m, 7H), 1.81-1.64 (m, 1H), 1.50-1.29 (m, 1H), 1.25-1.15 (m, 1H).

### N-[[4-[[1-(2,6-dioxo-3-piperidyl)-3-methyl-2-oxo-benzimidazol-5-yl]amino]phenyl]methyl]-5-fluoro-7-hydroxy-6-(1,1,4-trioxo-1,2,5-thiadiazolidin-2-yl)naphthalene-2-carboxamide (Example 148)

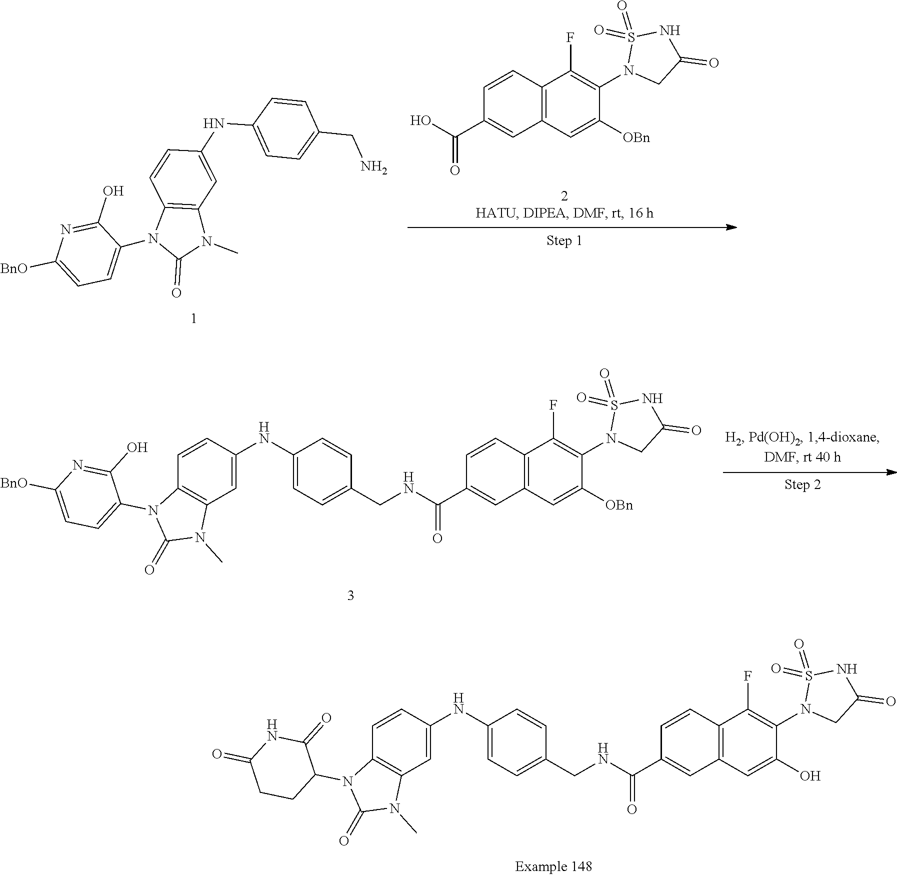 [SMILES: Cn1c(=O)n(-c2ccc(OCc3ccccc3)nc2O)c2ccc(Nc3ccc(CN)cc3)cc21 ; O=C1CN(c2c(OCc3ccccc3)cc3cc(C(=O)O)ccc3c2F)S(=O)(=O)N1 ; Cn1c(=O)n(-c2ccc(OCc3ccccc3)nc2O)c2ccc(Nc3ccc(CNC(=O)c4ccc5c(F)c(N6CC(=O)NS6(=O)=O)c(OCc6ccccc6)cc5c4)cc3)cc21 ; Cn1c(=O)n(C2CCC(=O)NC2=O)c2ccc(Nc3ccc(CNC(=O)c4ccc5c(F)c(N6CC(=O)NS6(=O)=O)c(O)cc5c4)cc3)cc21]

### Step 1: 7-benzyloxy-N-[[4-[[1-(6-benzyloxy-2-hydroxy-3-pyridyl)-3-methyl-2-oxo-benzimidazol-5-yl]amino]phenyl]methyl]-5-fluoro-6-(1,1,4-trioxo-1,2,5-thiadiazolidin-2-yl)naphthalene-2-carboxamide (3)

Into a 25 mL single neck round bottom flask containing well-stirred solution of 7-benzyloxy-5-fluoro-6-(1,1,4-trioxo-1,2,5-thiadiazolidin-2-yl)naphthalene-2-carboxylic acid (2, 270 mg, 587.29 μmol) in DMF (10 mL) were added DIPEA (759.03 mg, 5.87 mmol, 1.02 mL) and HATU (334.96 mg, 880.94 μmol). The solution was stirred at RT for 30 min. Then, 5-[4-(aminomethyl)anilino]-1-(6-benzyloxy-2-hydroxy-3-pyridyl)-3-methyl-benzimidazol-2-one (1, 359.17 mg, 587.29 μmol, TFA salt) was added. After 16 h, the reaction mixture was concentrated under reduced pressure and purified by reverse phase preparatory HPLC [Purification method: X Select C18 (19 mm×150 mm) 5 micron; Mobile phase A: 10% TFA in H₂O and Mobile phase B: MeCN] to afford 7-benzyloxy-N-[[4-[[1-(6-benzyloxy-2-hydroxy-3-pyridyl)-3-methyl-2-oxo-benzimidazol-5-yl]amino]phenyl]methyl]-5-fluoro-6-(1,1,4-trioxo-1,2,5-thiadiazolidin-2-yl)naphthalene-2-carboxamide (3, 220 mg, 201.64 μmol, 34% yield, TFA salt) as an off-white solid. LCMS (ES+): m/z 880.0 [M+H]⁺

### Step 2: N-[[4-[[1-(2,6-dioxo-3-piperidyl)-3-methyl-2-oxo-benzimidazol-5-yl]amino]phenyl]methyl]-5-fluoro-7-hydroxy-6-(1,1,4-trioxo-1,2,5-thiadiazolidin-2-yl)naphthalene-2-carboxamide (Example 148)

Into a 25 mL single neck round bottom flask containing a well-stirred solution of 7-benzyloxy-N-[[4-[[1-(6-benzyloxy-2-hydroxy-3-pyridyl)-3-methyl-2-oxo-benzimidazol-5-yl]amino]phenyl]methyl]-5-fluoro-6-(1,1,4-trioxo-1,2,5-thiadiazolidin-2-yl)naphthalene-2-carboxamide (3, 215 mg, 222.60 μmol) in a mixture of 1,4-dioxane (3 mL) and DMF (3 mL) was added palladium hydroxide (20 wt. % on carbon) (215 mg, 306.19 μmol) under nitrogen atmosphere. The suspension was stirred under bladder hydrogen atmosphere. After 40 h, the reaction mixture was filtered through Celite and washed with a mixture of DMF and 1,4-dioxane (15 mL/15 mL). The filtrate was concentrated under reduced pressure and triturated with MeCN (10 mL). The material was purified by reverse phase preparatory HPLC [Purification method: X-Bridge C18 (19 mm×150 mm) 5 micron; Mobile phase A: 0.1% TFA in H₂O and Mobile phase B: MeCN] to afford N-[[4-[[1-(2,6-dioxo-3-piperidyl)-3-methyl-2-oxo-benzimidazol-5-yl]amino]phenyl]methyl]-5-fluoro-7-hydroxy-6-(1,1,4-trioxo-1,2,5-thiadiazolidin-2-yl)naphthalene-2-carboxamide (Example 148, 31 mg, 35.73 μmol, 16% yield, TFA salt) as a light grey solid. LCMS (ES−): m/z 699.8 [M−H]⁻

¹H NMR (400 MHZ, DMSO-d₆) δ 11.08 (s, 1H), 10.61 (s, 1H), 9.11 (t, J=5.9 Hz, 1H), 8.31 (d, J=1.7 Hz, 1H), 7.98 (d, J=8.7 Hz, 1H), 7.83 (dd, J=8.8, 1.6 Hz, 1H), 7.23-7.17 (m, 3H), 6.98 (dd, J=8.5, 1.9 Hz, 3H), 6.89 (d, J=2.1 Hz, 1H), 6.75 (dd, J=8.4, 2.1 Hz, 1H), 5.31 (dd, J=12.8, 5.3 Hz, 1H), 4.44-4.37 (m, 4H), 3.28 (s, 3H), 2.96-2.83 (m, 1H), 2.75-2.54 (m, 3H), 2.05-1.96 (m, 1H).

### 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]ethyl]-4-piperidyl]-3-methyl-2-oxo-benzimidazol-1-yl]piperidine-2,6-dione (Example 149)

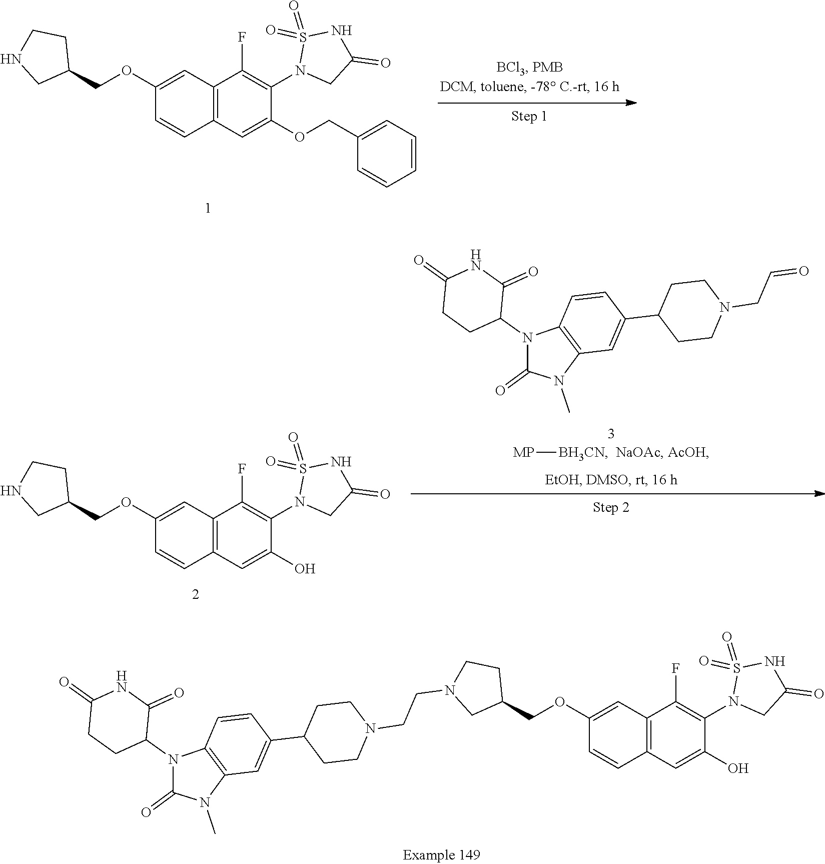

### Step 1: 5-[1-fluoro-3-hydroxy-7-[[(3R)-pyrrolidin-3-yl]methoxy]-2-naphthyl]-1,1-dioxo-1,2,5-thiadiazolidin-3-one (2)

Into a 50 mL round bottom flask containing a well-stirred solution of 5-[3-benzyloxy-1-fluoro-7-[[(3R)-pyrrolidin-3-yl]methoxy]-2-naphthyl]-1,1-dioxo-1,2,5-thiadiazolidin-3-one (1, 250 mg, 478.94 μmol, HCl salt) in toluene (2 mL) and DCM (2 mL) was added pentamethylbenzene (355.00 mg, 2.39 mmol, 387.13 μL) and the solution was cooled to −78° C. Boron trichloride (1.0 M in DCM) (1.12 g, 9.58 mmol, 9.5 mL) was added dropwise over a period of 2 min.

Subsequently the reaction mixture was stirred at RT. After 16 h, the reaction mixture was cooled to −78° C. and quenched with 10% methanol in DCM (3 mL). The reaction mixture was concentrated under reduced pressure at 30° C. The residue was washed with diethyl ether (10 mL) and filtered to afford 5-[1-fluoro-3-hydroxy-7-[[(3R)-pyrrolidin-3-yl]methoxy]-2-naphthyl]-1,1-dioxo-1,2,5-thiadiazolidin-3-one (2, 210 mg, 477.51 μmol, 100% yield). The material was taken to next step without purification. LCMS (ES+): m/z 396.0 [M+H]⁺

### Step 2: 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]ethyl]-4-piperidyl]-3-methyl-2-oxo-benzimidazol-1-yl]piperidine-2,6-dione (Example 149)

Into a 25 mL round bottom flask containing a well-stirred solution of 5-[1-fluoro-3-hydroxy-7-[[(3R)-pyrrolidin-3-yl]methoxy]-2-naphthyl]-1,1-dioxo-1,2,5-thiadiazolidin-3-one (2, 170 mg, 382.65 μmol) and 2-[4-[1-(2,6-dioxo-3-piperidyl)-3-methyl-2-oxo-benzimidazol-5-yl]-1-382.65 piperidyl]acetaldehyde (3, 262.68 mg, 382.65 μmol) in DMSO (2 mL) and ethanol (2 mL) were added sodium acetate (94.17 mg, 1.15 mmol, 61.55 μL) and acetic acid (229.78 mg, 3.83 mmol, 218.84 μL). After 1 h, MP-cyanoborohydride (382.65 mg, 765.29 μmol) was added. After 16 h, the reaction mixture was concentrated under reduced pressure and purified by reverse phase prep HPLC [Purification method: Column: X-Select, C18 (250×19) mm, 5 micron; Mobile phase A: 0.1% formic acid in water; Mobile phase B: MeCN] to afford 3-[5-[1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]ethyl]-4-piperidyl]-3-methyl-2-oxo-benzimidazol-1-yl]piperidine-2,6-dione (Example 149, 10 mg, 12.11 μmol, 3% yield, Formic acid salt) as an off-white solid. LCMS (ES+): m/z 764.1 [M+H]^{+ 1}H NMR (400 MHZ, DMSO-d₆) δ 11.10 (s, 1H), 9.53 (s, 1H), 7.69 (d, J=9.0 Hz, 1H), 7.25-7.21 (m, 1H), 7.15 (dd, J=9.0, 2.5 Hz, 1H), 7.07-7.01 (m, 3H), 6.89 (d, J=8.1 Hz, 1H), 5.34 (dd, J=12.8, 5.5 Hz, 1H), 4.17-4.02 (m, 4H), 3.20-3.11 (m, 2H), 3.04-2.79 (m, 6H), 2.62 (d, J=18.0 Hz, 2H), 2.21-2.11 (m, 2H), 2.05-1.93 (m, 2H), 1.91-1.73 (m, 5H), 1.30-1.10 (m, 2H), 0.89-0.76 (m, 1H). Note: additional peaks under solvent signal

### 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]ethyl]-4-piperidyl]-3-methyl-2-oxo-benzimidazol-1-yl]piperidine-2,6-dione (Example 150)

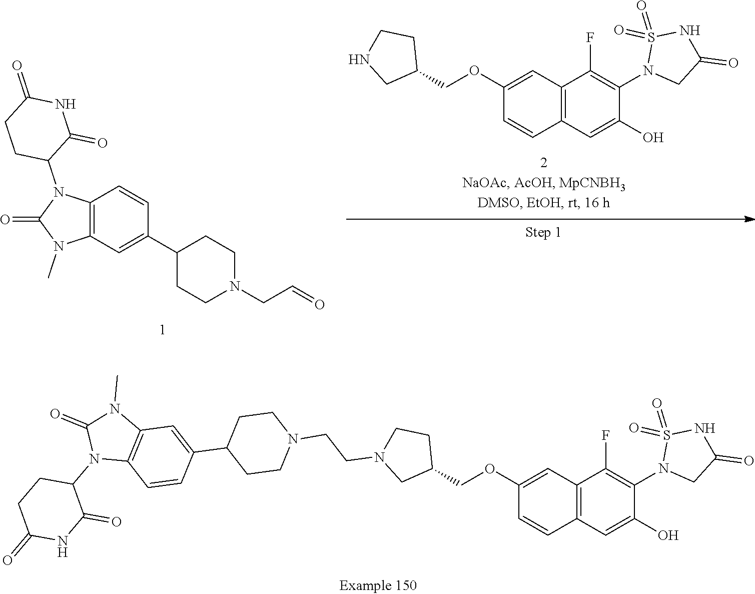 [SMILES: Cn1c(=O)n(C2CCC(=O)NC2=O)c2ccc(C3CCN(CC=O)CC3)cc21 ; O=C1CN(c2c(O)cc3ccc(OC[C@H]4CCNC4)cc3c2F)S(=O)(=O)N1 ; Cn1c(=O)n(C2CCC(=O)NC2=O)c2ccc(C3CCN(CCN4CC[C@H](COc5ccc6cc(O)c(N7CC(=O)NS7(=O)=O)c(F)c6c5)C4)CC3)cc21]

### Step 1: 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]ethyl]-4-piperidyl]-3-methyl-2-oxo-benzimidazol-1-yl]piperidine-2,6-dione (Example 150)

Into a 50 mL single neck round bottom flask containing a well-stirred solution of 2-[4-[1-(2,6-dioxo-3-piperidyl)-3-methyl-2-oxo-benzimidazol-5-yl]-1-piperidyl]acetaldehyde (1, 150 mg, 262.11 μmol, TFA salt) and 5-[1-fluoro-3-hydroxy-7-[[(3S)-pyrrolidin-3-yl]methoxy]-2-naphthyl]-1,1-dioxo-1,2,5-thiadiazolidin-3-one (2, 124.87 mg, 262.11 μmol) in anhydrous DMSO (2.0 mL) and ethanol (3.0 mL) were added sodium acetate (150.51 mg, 1.83 mmol), acetic acid (157.40 mg, 2.62 mmol, 149.90 μL) and MP-cyanoborohydride (2 mmol/g) (196.5 mg, 393.17 μmol). After 16 h, the mixture was filtered and concentrated under reduced pressure. The residue was purified by reverse phase prep HPLC [Purification method: X Select C18 (250×19) mm 5 microns column; Mobile phase A: 0.1% Formic acid in water; Mobile phase B: Acetonitrile] to afford of 3-[5-[1-[2-[(3S)-3-[[8-fluoro-6-hydroxy-7-(1,1,4-trioxo-1,2,5-thiadiazolidin-2-yl)-2-naphthyl]oxymethyl]pyrrolidin-1-yl]ethyl]-4-piperidyl]-3-methyl-2-oxo-benzimidazol-1-yl]piperidine-2,6-dione (Example 150, 17.3 mg, 8% yield, Formic acid salt) as a colorless solid.

LCMS (ES−): m/z 761.8 [M−H]⁻

¹H NMR (400 MHZ, DMSO-d₆) δ 11.09 (s, 1H), 7.68 (d, J=9.1, 1.5 Hz, 1H), 7.22 (d, J=2.6 Hz, 1H), 7.15 (dd, J=9.0, 2.5 Hz, 1H), 7.07-6.99 (m, 4H), 6.89 (dd, J=8.2, 1.6 Hz, 1H), 5.34 (dd, J=12.8, 5.4 Hz, 1H), 4.16-4.02 (m, 4H), 3.32 (s, 3H), 3.28-3.21 (m, 1H), 3.18-3.08 (m, 5H), 3.01-2.76 (m, 4H), 2.74-2.57 (m, 3H), 2.19-2.07 (m, 2H), 2.04-1.94 (m, 1H), 1.88-1.73 (m, 6H).

### 3-[5-[4-[[4-[[8-fluoro-6-hydroxy-7-(1,1,4-trioxo-1,2,5-thiadiazolidin-2-yl)-2-naphthyl]oxy]-2,2-dimethyl-butyl]amino]phenyl]-3-methyl-2-oxo-benzimidazol-1-yl]piperidine-2,6-dione (Example 151)

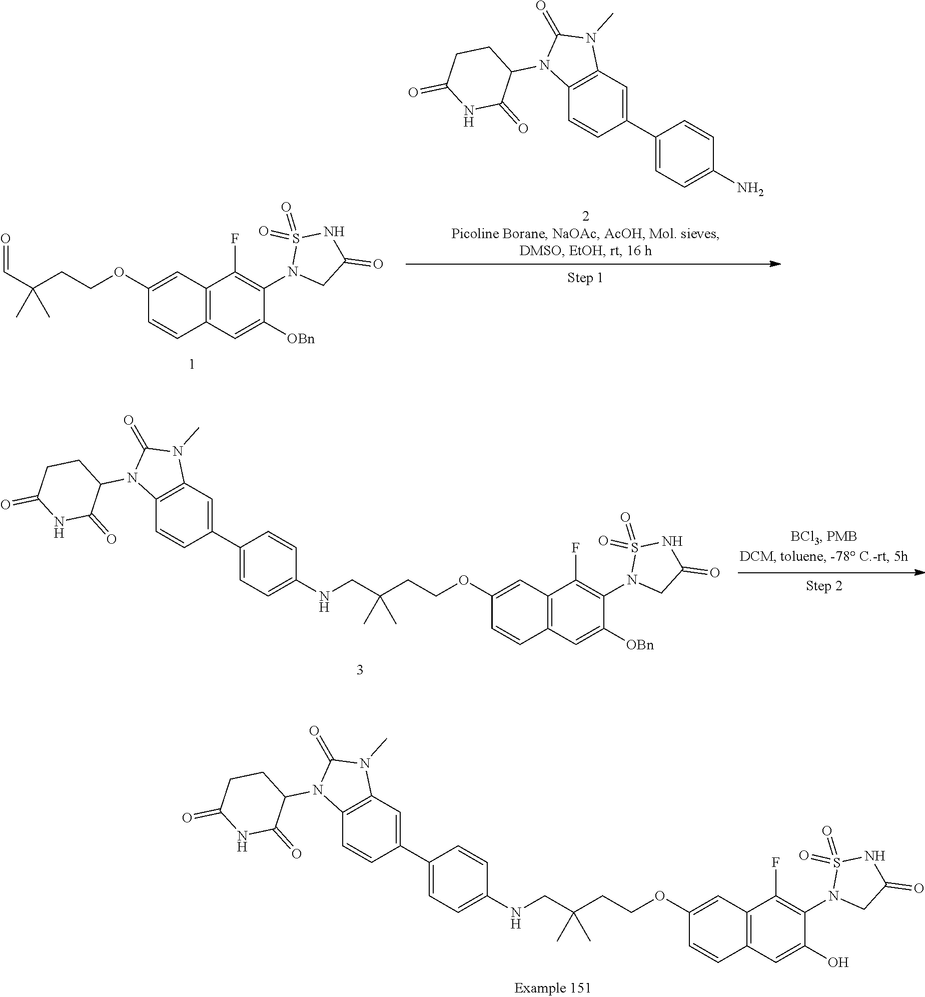 [SMILES: Cn1c(=O)n(C2CCC(=O)NC2=O)c2ccc(-c3ccc(N)cc3)cc21 ; CC(C)(C=O)CCOc1ccc2cc(OCc3ccccc3)c(N3CC(=O)NS3(=O)=O)c(F)c2c1 ; Cn1c(=O)n(C2CCC(=O)NC2=O)c2ccc(-c3ccc(NCC(C)(C)CCOc4ccc5cc(OCc6ccccc6)c(N6CC(=O)NS6(=O)=O)c(F)c5c4)cc3)cc21 ; Cn1c(=O)n(C2CCC(=O)NC2=O)c2ccc(-c3ccc(NCC(C)(C)CCOc4ccc5cc(O)c(N6CC(=O)NS6(=O)=O)c(F)c5c4)cc3)cc21]

### Step 1: 3-[5-[4-[[4-[[6-benzyloxy-8-fluoro-7-(1,1,4-trioxo-1,2,5-thiadiazolidin-2-yl)-2-naphthyl]oxy]-2,2-dimethyl-butyl]amino]phenyl]-3-methyl-2-oxo-benzimidazol-1-yl]piperidine-2,6-dione (3)

To a 25 single neck round bottom flask containing a solution of 3-[5-(4-aminophenyl)-3-methyl-2-oxo-benzimidazol-1-yl]piperidine-2,6-dione (2, 170 mg, 311.16 μmol, TFA salt) and 4-[[6-benzyloxy-8-fluoro-7-(1,1,4-trioxo-1,2,5-thiadiazolidin-2-yl)-2-naphthyl]oxy]-2,2-dimethyl-butanal (1, 232.46 mg, 311.16 μmol) in a mixture of ethanol (5 mL) and DMSO (1.5 mL) were added sodium acetate (51.05 mg, 622.32 μmol), acetic acid (3.74 mg, 62.23 μmol, 3.56 μL) and 4 Å molecular sieves (75 mg). After 30 min, picoline borane (33.28 mg, 311.16 μmol) was added. After 16 h, the reaction mixture was filtered, concentrated under reduced pressure and purified by reverse phase column chromatography [Purification method: Biotage C18 120 g column; Mobile phase A: 0.1% TFA in water; Mobile phase B: MeCN] to obtain 3-[5-[4-[[4-[[6-benzyloxy-8-fluoro-7-(1,1,4-trioxo-1,2,5-thiadiazolidin-2-yl)-2-naphthyl]oxy]-2,2-dimethyl-butyl]amino]phenyl]-3-methyl-2-oxo-benzimidazol-1-yl]piperidine-2,6-dione (3, 110 mg, 95.05 μmol, 31% yield, TFA salt) as a black sticky solid. LCMS (ES+): m/z 835 [M+H]⁺

### Step 2: 3-[5-[4-[[4-[[8-fluoro-6-hydroxy-7-(1,1,4-trioxo-1,2,5-thiadiazolidin-2-yl)-2-naphthyl]oxy]-2,2-dimethyl-butyl]amino]phenyl]-3-methyl-2-oxo-benzimidazol-1-yl]piperidine-2,6-dione (Example 151)

To a 50 mL single neck round bottom flask containing a suspension of 3-[5-[4-[[4-[[6-benzyloxy-8-fluoro-7-(1,1,4-trioxo-1,2,5-thiadiazolidin-2-yl)-2-naphthyl]oxy]-2,2-dimethyl-butyl]amino]phenyl]-3-methyl-2-oxo-benzimidazol-1-yl]piperidine-2,6-dione (3, 110 mg, 95.05 μmol, TFA salt) in DCM (3 mL) and toluene (3 mL), was added pentamethylbenzene (70.46 mg, 475.27 μmol) and the mixture was cooled to −78° C. After 5 min, boron trichloride (1.0 M in DCM) (222.73 mg, 1.90 mmol, 1.9 mL) was added and the reaction mixture stirred at ambient temperature. After 4 h, the reaction mixture was cooled to −78° C. and quenched with 5% MeOH in DCM (1 mL). The mixture was concentrated under reduced pressure and the residue was triturated with diethyl ether (40 mL). The material was purified by reverse phase prep HPLC [Purification method: Column: X Select C18 (150×30) mm, 5 micron; Mobile Phase A: 0.1% TFA in water; Mobile phase B: MeCN] to obtain 3-[5-[4-[[4-[[8-fluoro-6-hydroxy-7-(1,1,4-trioxo-1,2,5-thiadiazolidin-2-yl)-2-naphthyl]oxy]-2,2-dimethyl-butyl]amino]phenyl]-3-methyl-2-oxo-benzimidazol-1-yl]piperidine-2,6-dione (Example 151, 38 mg, 43.32 μmol, 46% yield, TFA salt) as a pale brown solid. LCMS (ES+): m/z 745.2 [M+H]⁺

¹H NMR (400 MHZ, DMSO-d₆) δ 11.11 (s, 1H), 10.20 (s, 1H), 7.69 (dd, J=9.1, 1.5 Hz, 1H), 7.44-7.41 (m, 2H), 7.36 (d, J=1.7 Hz, 1H), 7.27-7.20 (m, 2H), 7.14 (dd, J=9.0, 2.5 Hz, 1H), 7.10 (d, J=8.3 Hz, 1H), 7.05 (s, 1H), 6.78 (d, J=8.8 Hz, 2H), 5.37 (dd, J=12.8, 5.4 Hz, 1H), 4.42 (s, 2H), 4.17 (t, J=7.2 Hz, 2H), 3.38 (s, 3H), 2.99 (s, 2H), 2.91 (ddd, J=16.6, 13.3, 5.3 Hz, 1H), 2.78-2.58 (m, 2H), 2.07-1.98 (m, 1H), 1.86 (t, J=7.1 Hz, 2H), 1.06 (s, 6H).

### 3-[6-[1-[2-[4-[5-fluoro-7-hydroxy-6-(1,1,4-trioxo-1,2,5-thiadiazolidin-2-yl)-2-naphthyl]pyrazol-1-yl]ethyl]-4-piperidyl]-1-methyl-indazol-3-yl]piperidine-2,6-dione (Example 152)

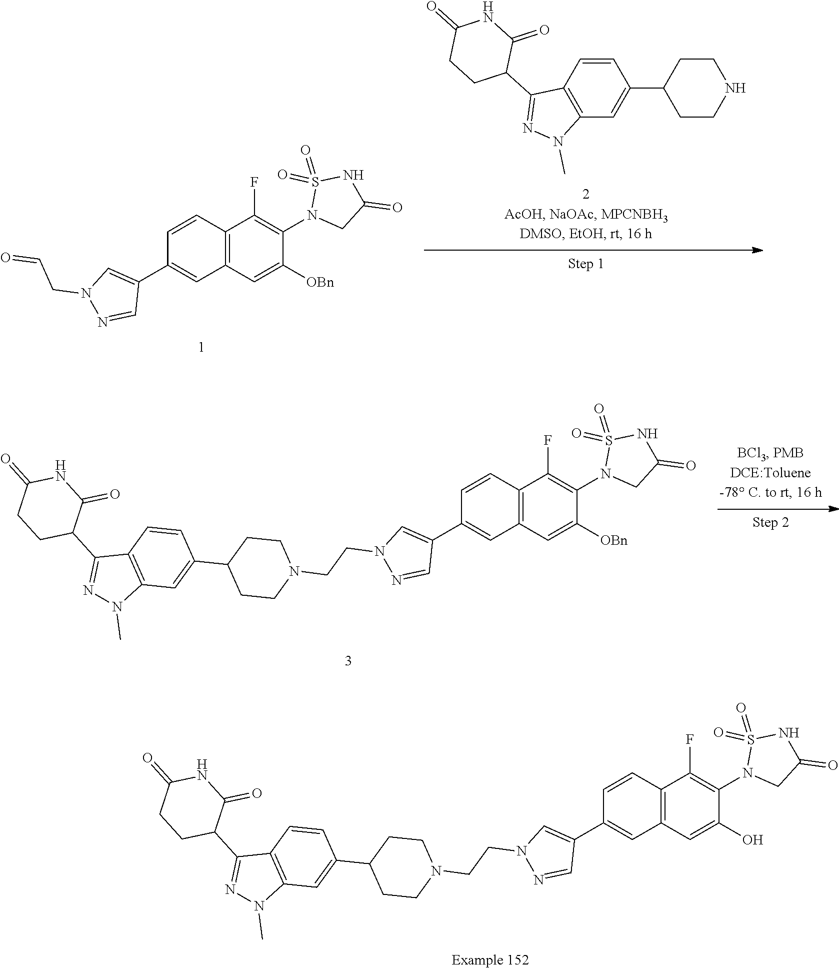 [SMILES: O=CCn1cc(-c2ccc3c(F)c(N4CC(=O)NS4(=O)=O)c(OCc4ccccc4)cc3c2)cn1 ; Cn1nc(C2CCC(=O)NC2=O)c2ccc(C3CCNCC3)cc21 ; Cn1nc(C2CCC(=O)NC2=O)c2ccc(C3CCN(CCn4cc(-c5ccc6c(F)c(N7CC(=O)NS7(=O)=O)c(OCc7ccccc7)cc6c5)cn4)CC3)cc21 ; Cn1nc(C2CCC(=O)NC2=O)c2ccc(C3CCN(CCn4cc(-c5ccc6c(F)c(N7CC(=O)NS7(=O)=O)c(O)cc6c5)cn4)CC3)cc21]

### Step 1: 3-[6-[1-[2-[4-[7-benzyloxy-5-fluoro-6-(1,1,4-trioxo-1,2,5-thiadiazolidin-2-yl)-2-naphthyl]pyrazol-1-yl]ethyl]-4-piperidyl]-1-methyl-indazol-3-yl]piperidine-2,6-dione (3)

Into a 50 mL single neck round bottom flask containing a well-stirred solution of 2-[4-[7-benzyloxy-5-fluoro-6-(1,1,4-trioxo-1,2,5-thiadiazolidin-2-yl)-2-naphthyl]pyrazol-1-yl]acetaldehyde (1, 270 mg, 416.19 μmol, TFA salt) and 3-[1-methyl-6-(4-piperidyl)indazol-3-yl]piperidine-2,6-dione (2, 184.78 mg, 416.19 μmol, TFA salt) in anhydrous DMSO (2 mL) and ethanol (4 mL) were added anhydrous sodium acetate (238.99 mg, 2.91 mmol), acetic acid (249.93 mg, 4.16 mmol, 238.03 μL) and MP-cyanoborohydride (2 mmol/1 g) (416 mg, 832.38 μmol). After 16 h, the mixture was filtered, the solvent removed under reduced pressure and purified by reverse phase column chromatography [Purification method: Biotage 120 g C18 column; Mobile phase A: 0.1% TFA in water; Mobile phase B: acetonitrile] to afford 3-[6-[1-[2-[4-[7-benzyloxy-5-fluoro-6-(1,1,4-trioxo-1,2,5-thiadiazolidin-2-yl)-2-naphthyl]pyrazol-1-yl]ethyl]-4-piperidyl]-1-methyl-indazol-3-yl]piperidine-2,6-dione (3, 170 mg, 39% yield, TFA salt) as a brown gummy solid. LCMS (ES+): m/z: 805.1 [M+H]⁺

### Step 2: 3-[6-[1-[2-[4-[5-fluoro-7-hydroxy-6-(1,1,4-trioxo-1,2,5-thiadiazolidin-2-yl)-2-naphthyl]pyrazol-1-yl]ethyl]-4-piperidyl]-1-methyl-indazol-3-yl]piperidine-2,6-dione (Example 152)

Into a 25 mL single neck round bottom flask containing a well-stirred solution of 3-[6-[1-[2-[4-[7-benzyloxy-5-fluoro-6-(1,1,4-trioxo-1,2,5-thiadiazolidin-2-yl)-2-naphthyl]pyrazol-1-yl]ethyl]-4-piperidyl]-1-methyl-indazol-3-yl]piperidine-2,6-dione (3, 170 mg, 157.25 μmol, TFA salt) in anhydrous 1,2-DCE (2 mL) and toluene (2 mL) was added pentamethylbenzene (116.56 mg, 786.26 μmol). The mixture was cooled to −78° C. and boron trichloride (1.0 M in DCM) (368.50 mg, 3.15 mmol, 3.15 mL) was added. The reaction mixture was stirred at ambient temperature for 16 h. The reaction was quenched with 5% MeOH in DCM at −78° C. (5 mL), volatiles were removed and the residue was triturated with diethyl ether (20 mL), filtered and dried. The crude compound was purified by reverse phase prep HPLC [Purification method: X Select C18 (250×19) mm 5 microns; Mobile phase A: 0.1% formic acid in water; Mobile phase B: acetonitrile] to afford 3-[6-[1-[2-[4-[5-fluoro-7-hydroxy-6-(1,1,4-trioxo-1,2,5-thiadiazolidin-2-yl)-2-naphthyl]pyrazol-1-yl]ethyl]-4-piperidyl]-1-methyl-indazol-3-yl]piperidine-2,6-dione (Example 152, 33 mg, 27% yield, formic acid salt) as an off-white solid. LCMS (ES+): m/z 715.3 [M+H]^{+ 1}H NMR (400 MHZ, DMSO-d₆) δ 10.90 (s, 1H), 9.85 (s, 1H), 9.32 (s, 1H), 8.46 (s, 1H), 8.19 (s, 1H), 7.97 (s, 1H), 7.89 (d, J=8.6 Hz, 1H), 7.70-7.58 (m, 2H), 7.42 (s, 1H), 7.08-6.99 (m, 2H), 4.64 (s, 2H), 4.33 (dd, J=9.9, 5.0 Hz, 1H), 4.09 (s, 2H), 3.98 (s, 3H), 3.77-3.62 (m, 2H), 3.24-3.12 (m, 1H), 3.03-2.86 (m, 1H), 2.73-2.56 (m, 2H), 2.42-2.34 (m, 1H), 2.21-1.89 (m, 5H).

### 3-[5-[4-[[[4-[[8-fluoro-6-hydroxy-7-(1,1,4-trioxo-1,2,5-thiadiazolidin-2-yl)-2-naphthyl]oxy]-2,2-dimethyl-butyl]amino]methyl]phenyl]-3-methyl-2-oxo-benzimidazol-1-yl]piperidine-2,6-dione (Example 153)

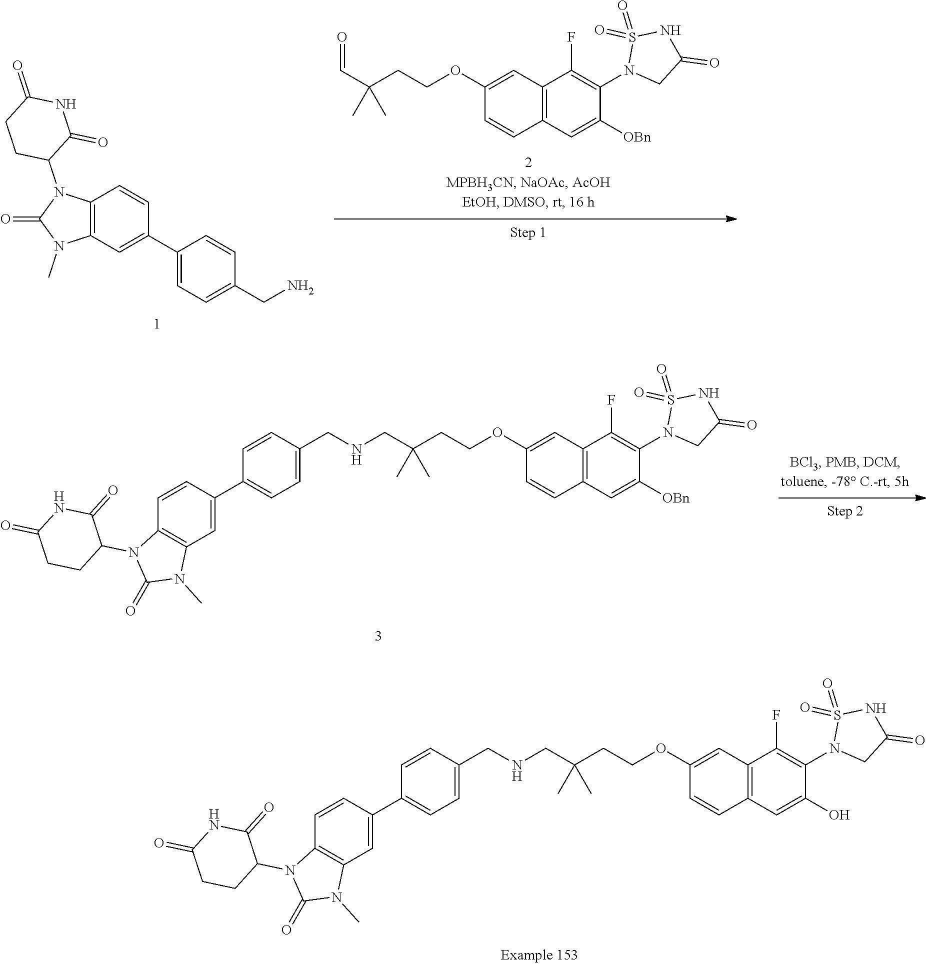 [SMILES: Cn1c(=O)n(C2CCC(=O)NC2=O)c2ccc(-c3ccc(CN)cc3)cc21 ; CC(C)(C=O)CCOc1ccc2cc(OCc3ccccc3)c(N3CC(=O)NS3(=O)=O)c(F)c2c1 ; Cn1c(=O)n(C2CCC(=O)NC2=O)c2ccc(-c3ccc(CNCC(C)(C)CCOc4ccc5cc(OCc6ccccc6)c(N6CC(=O)NS6(=O)=O)c(F)c5c4)cc3)cc21 ; Cn1c(=O)n(C2CCC(=O)NC2=O)c2ccc(-c3ccc(CNCC(C)(C)CCOc4ccc5cc(O)c(N6CC(=O)NS6(=O)=O)c(F)c5c4)cc3)cc21]

### Step 1: 3-[5-[4-[[[4-[[6-benzyloxy-8-fluoro-7-(1,1,4-trioxo-1,2,5-thiadiazolidin-2-yl)-2-naphthyl]oxy]-2,2-dimethyl-butyl]amino]methyl]phenyl]-3-methyl-2-oxo-benzimidazol-1-yl]piperidine-2,6-dione (3)

In a 50 mL single neck round bottom flask containing a well-stirred solution of 4-[[6-benzyloxy-8-fluoro-7-(1,1,4-trioxo-1,2,5-thiadiazolidin-2-yl)-2-naphthyl]oxy]-2,2-dimethyl-butanal (1, 270 mg, 419.13 μmol) in ethanol (3 mL) mL) were added 3-[5-[4-(aminomethyl)phenyl]-3-methyl-2-oxo-benzimidazol-1-yl]piperidine-2,6-dione (2, 150 mg, 292.26 μmol, TFA salt), sodium acetate (47.95 mg, 584.53 μmol, 31.34 μL) and acetic acid (210.00 mg, 3.50 mmol, 0.2 mL). After 15 min, MP-cyanoborohydride (2.0 mmol/g) (200 mg, 584.53 μmol) was added. After 16 h, the reaction mixture was filtered and concentrated under reduced pressure. The residue was purified by reverse phase column chromatography [Purification method: Siliasep C18 120 g column; Mobile phase A: 0.1% TFA in water; Mobile phase B: MeCN] to afford 3-[5-[4-[[[4-[[6-benzyloxy-8-fluoro-7-(1,1,4-trioxo-1,2,5-thiadiazolidin-2-yl)-2-naphthyl]oxy]-2,2-dimethyl-butyl]amino]methyl]phenyl]-3-methyl-2-oxo-benzimidazol-1-yl]piperidine-2,6-dione (3, 170 mg, 153.06 μmol, 52% yield) as a pale yellow solid. LCMS (ES+): m/z 849.1 [M+H]⁺

### Step 2: 3-[5-[4-[[[4-[[8-fluoro-6-hydroxy-7-(1,1,4-trioxo-1,2,5-thiadiazolidin-2-yl)-2-naphthyl]oxy]-2,2-dimethyl-butyl]amino]methyl]phenyl]-3-methyl-2-oxo-benzimidazol-1-yl]piperidine-2,6-dione (Example 153)

In a 50 mL single neck round bottom flask containing a well-stirred solution of 3-[5-[4-[[[4-[[6-benzyloxy-8-fluoro-7-(1,1,4-trioxo-1,2,5-thiadiazolidin-2-yl)-2-naphthyl]oxy]-2,2-dimethyl-butyl]amino]methyl]phenyl]-3-methyl-2-oxo-benzimidazol-1-yl]piperidine-2,6-dione (3, 170 mg, 173.62 μmol) in toluene (3 mL) and DCM (3 mL) was added pentamethylbenzene (128.69 mg, 868.09 μmol, 140.34 μL). The suspension was cooled to −78° C. and treated with boron trichloride (1.0 M in DCM) (3.47 mmol, 3.4 mL). The reaction mixture was then stirred at RT for 5 h. The reaction mixture was quenched with 5% MeOH in DCM (2 mL) at −75° C. The reaction mixture was concentrated under reduced pressure, triturated with diethyl ether (10 mL) and purified by reverse phase prep HPLC [Purification method: Column: Zoxbax C18 (250×21 mm), 7 micron; Mobile phase A: 0.1% TFA in water; Mobile phase B: MeCN] to afford 3-[5-[4-[[[4-[[8-fluoro-6-hydroxy-7-(1,1,4-trioxo-1,2,5-thiadiazolidin-2-yl)-2-naphthyl]oxy]-2,2-dimethyl-butyl]amino]methyl]phenyl]-3-methyl-2-oxo-benzimidazol-1-yl]piperidine-2,6-dione (Example 153, 34 mg, 43.64 μmol, 25% yield. TFA Salt) as an off-white solid. LCMS (ES+): m/z 759.2 [M+H]⁺

¹H NMR (400 MHZ, DMSO-d6): δ 11.14 (s, 1H), 9.98 (s, 1H), 8.64 (s, 2H), 7.74 (d, J=8.00 Hz, 2H), 7.69-7.58 (m, 3H), 7.44 (m, 1H), 7.32-7.30 (m, 1H), 7.20-7.11 (m, 2H), 7.10-7.01 (m, 2H), 5.38-5.33 (m, 1H), 4.22-4.18 (m, 4H), 4.06-4.00 (m, 2H), 3.38 (s, 3H), 2.93-2.88 (m, 3H), 2.74-2.65 (m, 2H), 2.09-2.06 (m, 1H), 1.83-1.79 (m, 2H), 1.04 (s, 6H).

### 3-(5-(1-((1-(6-(1,1-dioxido-4-oxo-1,2,5-thiadiazolidin-2-yl)-5-fluoro-7-hydroxynaphthalen-2-yl)-1H-pyrazol-3-yl)methyl)piperidin-4-yl)-3-methyl-2-oxo-2,3-dihydro-1H-benzo[d]imidazol-1-yl)piperidine-2,6-dione (Example 154)

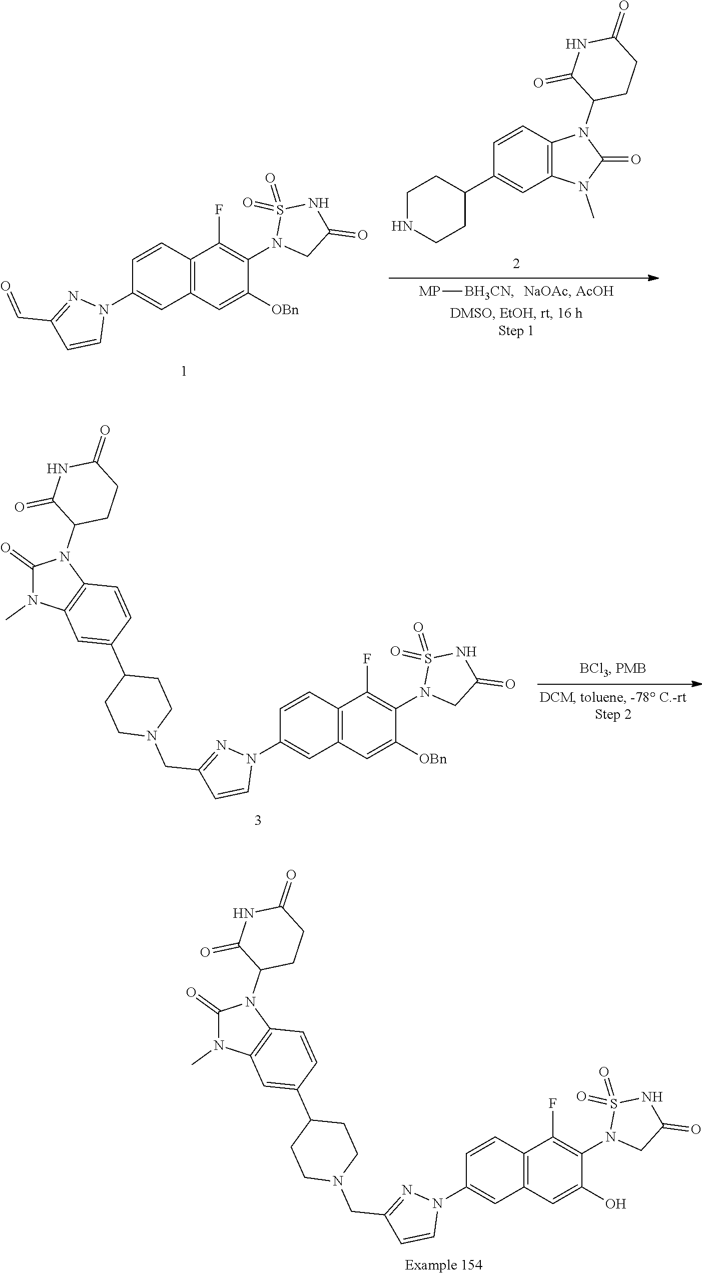

### Step 1: 3-(5-(1-((1-(7-(benzyloxy)-6-(1,1-dioxido-4-oxo-1,2,5-thiadiazolidin-2-yl)-5-fluoronaphthalen-2-yl)-1H-pyrazol-3-yl)methyl)piperidin-4-yl)-3-methyl-2-oxo-2,3-dihydro-1H-benzo[d]imidazol-1-yl)piperidine-2,6-dione (3)

Into a 25 mL single neck round bottom flask containing a well-stirred solution of 1-[7-benzyloxy-5-fluoro-6-(1,1,4-trioxo-1,2,5-thiadiazolidin-2-yl)-2-naphthyl]pyrazole-3-carbaldehyde (1, 280 mg, 332.18 μmol) in DMSO (4 mL) and ethanol (4 mL) were added 3-[3-methyl-2-oxo-5-(4-piperidyl)benzimidazol-1-yl]piperidine-2,6-dione (2, 157.93 mg, 332.18 μmol, TFA salt), acetic acid (199.47 mg, 3.32 mmol, 189.97 μL), sodium acetate (81.75 mg, 996.53 μmol, 53.43 μL) and MP-cyanoborohydride (2 mmol in 1 g) (498.21 mg, 665.53 μmol). After 16 h, the reaction mixture was filtered, concentrated and purified by reverse phase column chromatography [Purification method: Siliasep premium C18, 25 μm, 120 g column; Mobile phase A: 0.1% TFA in water; Mobile phase B: Acetonitrile] to afford 3-(5-(1-((1-(7-(benzyloxy)-6-(1,1-dioxido-4-oxo-1,2,5-thiadiazolidin-2-yl)-5-fluoronaphthalen-2-yl)-1H-pyrazol-3-yl)methyl)piperidin-4-yl)-3-methyl-2-oxo-2,3-dihydro-1H-benzo[d]imidazol-1-yl)piperidine-2,6-dione (3, 200 mg, 193.01 μmol, 58% yield, TFA salt) as a pale yellow solid. LCMS (ES+): m/z 807.1 [M+H]⁺

### Step 2: 3-(5-(1-((1-(6-(1,1-dioxido-4-oxo-1,2,5-thiadiazolidin-2-yl)-5-fluoro-7-hydroxynaphthalen-2-yl)-1H-pyrazol-3-yl)methyl)piperidin-4-yl)-3-methyl-2-oxo-2,3-dihydro-1H-benzo[d]imidazol-1-yl)piperidine-2,6-dione (Example 154)

Into a 50 mL round bottom flask containing a well-stirred solution of 3-(5-(1-((1-(7-(benzyloxy)-6-(1,1-dioxido-4-oxo-1,2,5-thiadiazolidin-2-yl)-5-fluoronaphthalen-2-yl)-1H-pyrazol-3-yl)methyl)piperidin-4-yl)-3-methyl-2-oxo-2,3-dihydro-1H-benzo[d]imidazol-1-yl)piperidine-2,6-dione (3, 200 mg, 218.13 μmol) in DCM (3 mL) and toluene (3 mL) was added pentamethylbenzene (161.68 mg, 1.09 mmol, 176.32 μL). The suspension was cooled to −78° C. and treated with boron trichloride (1.0 M in DCM) (4.36 mmol, 4.3 mL). The reaction mixture was stirred at RT and stirred for 5 h. The reaction mixture was quenched with 5% MeOH in DCM (2.5 mL) at −75° C. The reaction mixture was concentrated under reduced pressure and the residue was triturated with diethyl ether (10 mL). The material was purified by reverse phase prep HPLC [Purification method: Column: X-select, C18 (150×19) mm, 5 micron; Mobile phase A: 0.1% TFA in water; Mobile phase B: MeCN] to afford 3-(5-(1-((1-(6-(1,1-dioxido-4-oxo-1,2,5-thiadiazolidin-2-yl)-5-fluoro-7-hydroxynaphthalen-2-yl)-1H-pyrazol-3-yl)methyl)piperidin-4-yl)-3-methyl-2-oxo-2,3-dihydro-1H-benzo[d]imidazol-1-yl)piperidine-2,6-dione (Example 154, 95 mg, 113.66 μmol, 52% yield, TFA salt) as an off white solid. LCMS (ES+): m/z 717.3 [M+H]⁺

¹H NMR (400 MHZ, DMSO-d₆) δ 11.10 (s, 1H), 10.18 (s, 1H), 9.82 (s, 1H), 8.78 (d, J=2.5 Hz, 1H), 8.22 (d, J=2.0 Hz, 1H), 8.06 (d, J=9.1 Hz, 1H), 7.95 (dd, J=9.1, 2.1 Hz, 1H), 7.15 (s, 1H), 7.09-7.01 (m, 2H), 6.90 (dd, J=8.2, 1.6 Hz, 1H), 6.81 (d, J=2.5 Hz, 1H), 5.35 (dd, J=12.8, 5.4 Hz, 1H), 4.50 (d, J=4.4 Hz, 2H), 4.13 (s, 2H), 3.64 (d, J=11.8 Hz, 2H), 3.33 (s, 3H), 3.26-3.14 (m, 2H), 2.96-2.82 (m, 2H), 2.75-2.56 (m, 2H), 2.12-1.88 (m, 5H).

### 3-[5-[4-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-hydroxy-1-piperidyl]-3-methyl-2-oxo-benzimidazol-1-yl]piperidine-2,6-dione (Example 155)

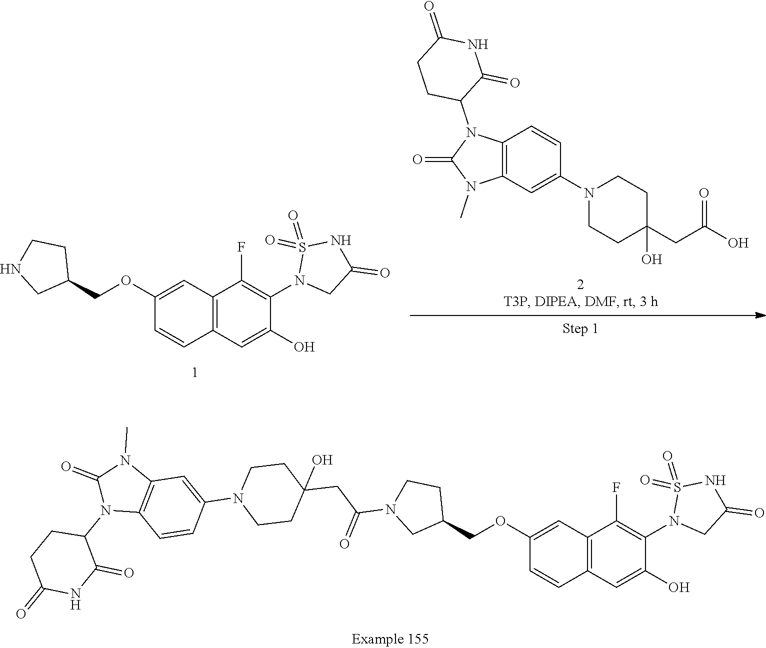 [SMILES: O=C1CN(c2c(O)cc3ccc(OC[C@@H]4CCNC4)cc3c2F)S(=O)(=O)N1 ; Cn1c(=O)n(C2CCC(=O)NC2=O)c2ccc(N3CCC(O)(CC(=O)O)CC3)cc21 ; Cn1c(=O)n(C2CCC(=O)NC2=O)c2ccc(N3CCC(O)(CC(=O)N4CC[C@@H](COc5ccc6cc(O)c(N7CC(=O)NS7(=O)=O)c(F)c6c5)C4)CC3)cc21]

### Step 1: 3-[5-[4-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-hydroxy-1-piperidyl]-3-methyl-2-oxo-benzimidazol-1-yl]piperidine-2,6-dione (Example 155)

Into a 50 mL single neck round bottom flask containing a well-stirred solution of 2-[1-[1-(2,6-dioxo-3-piperidyl)-3-methyl-2-oxo-benzimidazol-5-yl]-4-hydroxy-4-piperidyl]acetic acid (2, 200 mg, 324.25 μmol, TFA salt) in anhydrous DMF (7 mL) were added propylphosphonic anhydride (≥50 wt. % in ethyl acetate) (412.68 mg, 648.51 μmol, 389.32 μL, 50% purity) and DIPEA (251.44 mg, 1.95 mmol, 338.87 μL). After 10 min, 5-[1-fluoro-3-hydroxy-7-[[(3R)-pyrrolidin-3-yl]methoxy]-2-naphthyl]-1,1-dioxo-1,2,5-thiadiazolidin-3-one (1, 151.75 mg, 356.68 μmol) was added. After 3 h, the solvent was removed under reduced pressure and the residue was subjected to reverse phase prep HPLC [Purification method: Column: X-Bridge C18 (150×19) 5 micron; Mobile phase A: 0.1% TFA in water; Mobile phase B: MeCN] to afford 3-[5-[4-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-hydroxy-1-piperidyl]-3-methyl-2-oxo-benzimidazol-1-yl]piperidine-2,6-dione (Example 155, 55 mg, 58.21 μmol, 18% yield, TFA salt) as an off-white solid. LCMS (ES+): m/z 794.2 [M+H]⁺

¹H NMR (400 MHZ, DMSO-d₆) δ 11.14 (s, 1H), 9.96 (s, 1H), 7.70 (d, J=9.0 Hz, 1H), 7.51 (s, 1H), 7.25-7.15 (m, 4H), 7.07-7.03 (m, 1H), 5.45-5.31 (m, 1H), 4.29 (d, J=3.9 Hz, 2H), 4.18-4.02 (m, 3H), 3.84-3.75 (m, 1H), 3.38 (d, J=1.6 Hz, 3H), 2.90 (ddd, J=17.0, 12.6, 5.1 Hz, 1H), 2.83-2.55 (m, 7H), 2.22-1.97 (m, 5H), 1.95-1.84 (m, 3H), 1.83-1.71 (m, 1H). Note: additional peaks under solvent signal

### 3-[5-[1-[2-[4-[8-fluoro-6-hydroxy-7-(1,1,4-trioxo-1,2,5-thiadiazolidin-2-yl)-2-naphthyl]piperazin-1-yl]-2-oxo-ethyl]-4-piperidyl]-3-methyl-2-oxo-benzimidazol-1-yl]piperidine-2,6-dione (Example 156)

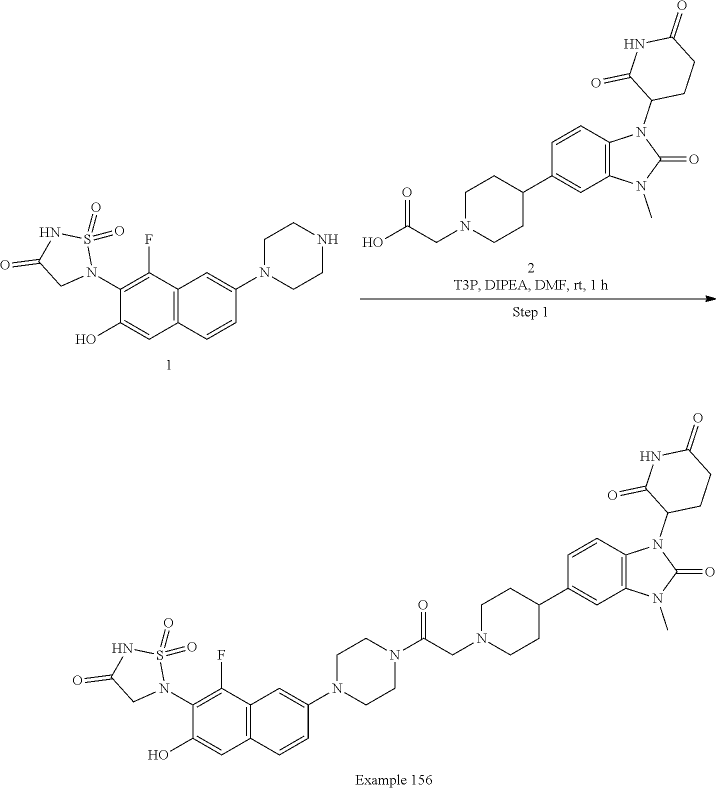 [SMILES: O=C1CN(c2c(O)cc3ccc(N4CCNCC4)cc3c2F)S(=O)(=O)N1 ; Cn1c(=O)n(C2CCC(=O)NC2=O)c2ccc(C3CCN(CC(=O)O)CC3)cc21 ; Cn1c(=O)n(C2CCC(=O)NC2=O)c2ccc(C3CCN(CC(=O)N4CCN(c5ccc6cc(O)c(N7CC(=O)NS7(=O)=O)c(F)c6c5)CC4)CC3)cc21]

### Step 1: 3-[5-[1-[2-[4-[8-fluoro-6-hydroxy-7-(1,1,4-trioxo-1,2,5-thiadiazolidin-2-yl)-2-naphthyl]piperazin-1-yl]-2-oxo-ethyl]-4-piperidyl]-3-methyl-2-oxo-benzimidazol-1-yl]piperidine-2,6-dione (Example 156)

Into a 25 mL single neck round bottom flask containing a well-stirred solution of 2-[4-[1-(2,6-dioxo-3-piperidyl)-3-methyl-2-oxo-benzimidazol-5-yl]-1-piperidyl]acetic acid (2, 90 mg, 167.95 μmol, TFA salt) in DMF (2 mL) were added DIPEA (108.53 mg, 839.73 μmol, 146.26 μL) and propylphosphonic anhydride (≥50 wt. % in ethyl acetate) (160.22 mg, 251.92 μmol, 50% purity). After 5 min, 5-(1-fluoro-3-hydroxy-7-piperazin-1-yl-2-naphthyl)-1,1-dioxo-1,2,5-thiadiazolidin-3-one (1, 82.68 mg, 184.74 μmol) was added. After 1 h, the volatiles were removed under reduced pressure and the residue was purified by reverse phase prep HPLC [Purification method: Column: X-Bridge C18 (19×150 mm), 5 mic; Mobile phase A: 0.1% TFA in water; Mobile phase B: MeCN] to afford 3-[5-[1-[2-[4-[8-fluoro-6-hydroxy-7-(1,1,4-trioxo-1,2,5-thiadiazolidin-2-yl)-2-naphthyl]piperazin-1-yl]-2-oxo-ethyl]-4-piperidyl]-3-methyl-2-oxo-benzimidazol-1-yl]piperidine-2,6-dione (Example 156, 86 mg, 92.06 μmol, 55% yield, TFA salt) as an off white solid. LCMS (ES+): m/z 763.2 [M+H]⁺

¹H NMR (400 MHZ, DMSO-d₆) δ 11.10 (s, 1H), 9.50 (s, 2H), 7.70-7.61 (m, 1H), 7.41 (dd, J=9.1, 2.4 Hz, 1H), 7.12-7.04 (m, 3H), 6.99 (s, 1H), 6.96-6.92 (m, 1H), 5.36 (dd, J=12.8, 5.4 Hz, 1H), 4.54-4.38 (m, 2H), 4.12 (s, 2H), 3.79-3.70 (m, 2H), 3.35 (d, J=2.9 Hz, 3H), 3.33-3.28 (m, 2H), 3.27-3.22 (m, 2H), 3.20-3.07 (m, 2H), 2.97-2.83 (m, 2H), 2.76-2.57 (m, 2H), 2.18-1.88 (m, 5H). Note: additional peaks under solvent signal

### 3-[5-[1-[4-[[8-fluoro-6-hydroxy-7-(1,1,4-trioxo-1,2,5-thiadiazolidin-2-yl)-2-naphthyl]oxy]-2,2-dimethyl-butyl]-4-piperidyl]-3-methyl-2-oxo-benzimidazol-1-yl]piperidine-2,6-dione (Example 157)

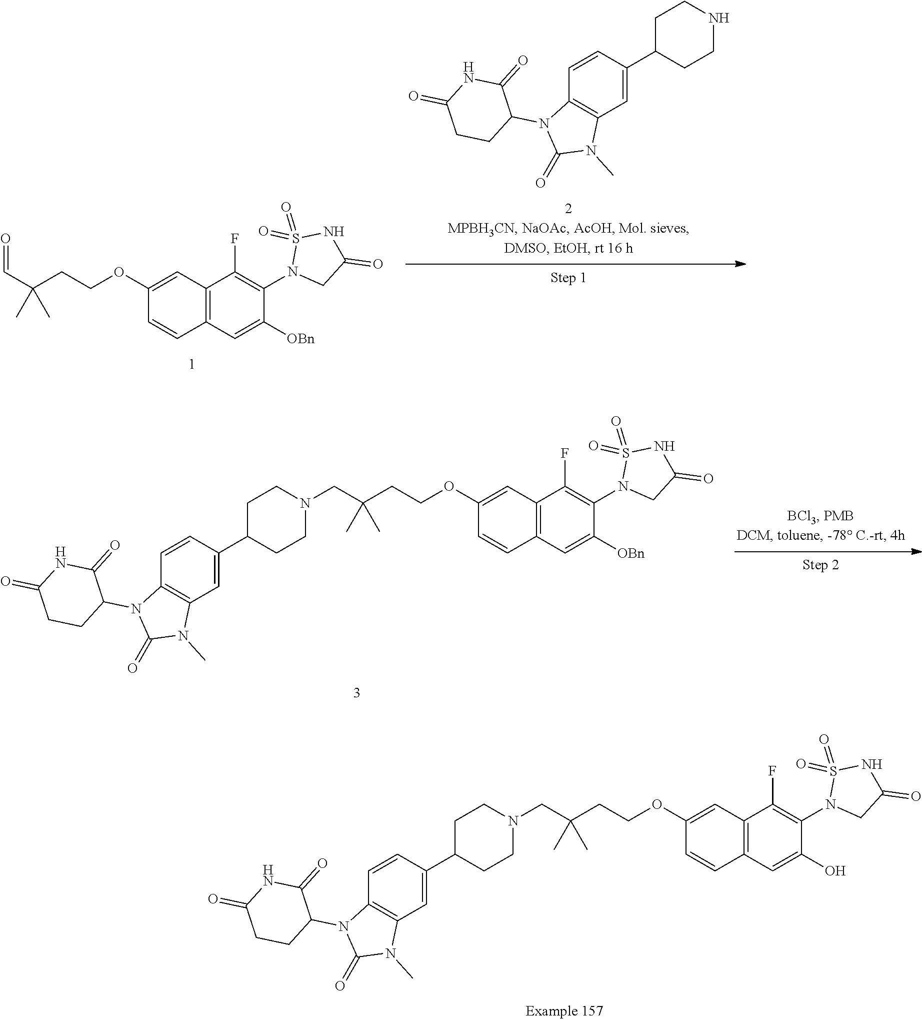 [SMILES: Cn1c(=O)n(C2CCC(=O)NC2=O)c2ccc(C3CCNCC3)cc21 ; CC(C)(C=O)CCOc1ccc2cc(OCc3ccccc3)c(N3CC(=O)NS3(=O)=O)c(F)c2c1 ; Cn1c(=O)n(C2CCC(=O)NC2=O)c2ccc(C3CCN(CC(C)(C)CCOc4ccc5cc(OCc6ccccc6)c(N6CC(=O)NS6(=O)=O)c(F)c5c4)CC3)cc21 ; Cn1c(=O)n(C2CCC(=O)NC2=O)c2ccc(C3CCN(CC(C)(C)CCOc4ccc5cc(O)c(N6CC(=O)NS6(=O)=O)c(F)c5c4)CC3)cc21]

### Step 1: 3-[5-[1-[4-[[6-benzyloxy-8-fluoro-7-(1,1,4-trioxo-1,2,5-thiadiazolidin-2-yl)-2-naphthyl]oxy]-2,2-dimethyl-butyl]-4-piperidyl]-3-methyl-2-oxo-benzimidazol-1-yl]piperidine-2,6-dione (3)

To a 50 mL single neck round bottom flask containing a solution of 3-[3-methyl-2-oxo-5-(4-piperidyl)benzimidazol-1-yl]piperidine-2,6-dione (2, 183.28 mg, 393.54 μmol, TFA salt) in ethanol (8 mL) and DMSO (2 mL) was added sodium acetate (69.18 mg, 843.29 μmol). After 1 h, 4-[[6-benzyloxy-8-fluoro-7-(1,1,4-trioxo-1,2,5-thiadiazolidin-2-yl)-2-naphthyl]oxy]-2,2-dimethyl-butanal (1, 210 mg, 281.10 μmol), acetic acid (16.88 mg, 281.10 μmol, 16.08 μL) and 4 Å molecular sieves (100 mg) were added. After 1 h, MP-cyanoborohydride (281 mg, 562.20 μmol) was added. After 16 h, the reaction mixture was filtered, concentrated and purified by reverse phase column chromatography [Purification method: Biotage C18 120 g column; Mobile phase A: 0.1% TFA in water; Mobile phase B: MeCN] to obtain 3-[5-[1-[4-[[6-benzyloxy-8-fluoro-7-(1,1,4-trioxo-1,2,5-thiadiazolidin-2-yl)-2-naphthyl]oxy]-2,2-dimethyl-butyl]-4-piperidyl]-3-methyl-2-oxo-benzimidazol-1-yl]piperidine-2,6-dione (3, 37 mg, 28.31 μmol, 10.07% yield, 72% purity, TFA salt) as a dark brown solid. LCMS (ES+): m/z 827.1 [M+H]⁺

### Step 2: 3-[5-[1-[4-[[8-fluoro-6-hydroxy-7-(1,1,4-trioxo-1,2,5-thiadiazolidin-2-yl)-2-naphthyl]oxy]-2,2-dimethyl-butyl]-4-piperidyl]-3-methyl-2-oxo-benzimidazol-1-yl]piperidine-2,6-dione (Example 157)

To a 25 mL single neck round bottom flask containing suspension of 3-[5-[1-[4-[[6-benzyloxy-8-fluoro-7-(1,1,4-trioxo-1,2,5-thiadiazolidin-2-yl)-2-naphthyl]oxy]-2,2-dimethyl-butyl]-4-piperidyl]-3-methyl-2-oxo-benzimidazol-1-yl]piperidine-2,6-dione (3, 35 mg, 26.78 μmol, TFA salt) in a mixture of toluene (0.8 mL) and DCM (0.8 mL) was added pentamethylbenzene (19.85 mg, 133.91 μmol) under nitrogen atmosphere. The resulting suspension was cooled to −78° C. and treated with boron trichloride (1.0 M in DCM) (70.60 mg, 602.58 μmol, 0.6 mL) via dropwise addition. The reaction mixture was stirred at ambient temperature for 4 h. The reaction mixture was cooled to −78° C. and quenched with 5% MeOH in DCM (0.3 mL). The solution was immediately concentrated under vacuum at 35° C. The residue was triturated with diethyl ether (2×10 mL), filtered, dried under vacuum and purified by reverse phase prep HPLC [Purification method: Column: X-Bridge C18 (19×150 mm), 5 Micron: Mobile phase A: 0.1% TFA in water; Mobile phase B: MeCN] to obtain 3-[5-[1-[4-[[8-fluoro-6-hydroxy-7-(1,1,4-trioxo-1,2,5-thiadiazolidin-2-yl)-2-naphthyl]oxy]-2,2-dimethyl-butyl]-4-piperidyl]-3-methyl-2-oxo-benzimidazol-1-yl]piperidine-2,6-dione (Example 157, 2.5 mg, 2.91 μmol, 11% yield, TFA salt). LCMS (ES+): m/z 737.3 [M+H]⁺

¹H NMR (400 MHZ, DMSO-d₆) δ 11.10 (s, 1H), 9.55 (s, 1H), 8.23 (s, 1H), 7.71-7.66 (m, 1H), 7.29-7.26 (m, 1H), 7.16-7.12 (m, 1H), 7.10-7.06 (m, 1H), 7.05-7.02 (m, 1H), 6.96-6.91 (m, 1H), 5.36 (dd, J=12.7, 5.4 Hz, 1H), 4.21 (t, J=6.8 Hz, 2H), 4.11 (d, J=1.8 Hz, 2H), 3.64 (d, J=11.8 Hz, 1H), 3.20-3.18 (m, 2H), 2.97-2.80 (m, 3H), 2.76-2.57 (m, 3H), 2.24-2.10) (m, 2H), 2.03-1.85 (m, 5H), 1.18 (d, J=8.3 Hz, 6H). Note: additional peaks under solvent signal

### 3-[6-[1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (Example 158)

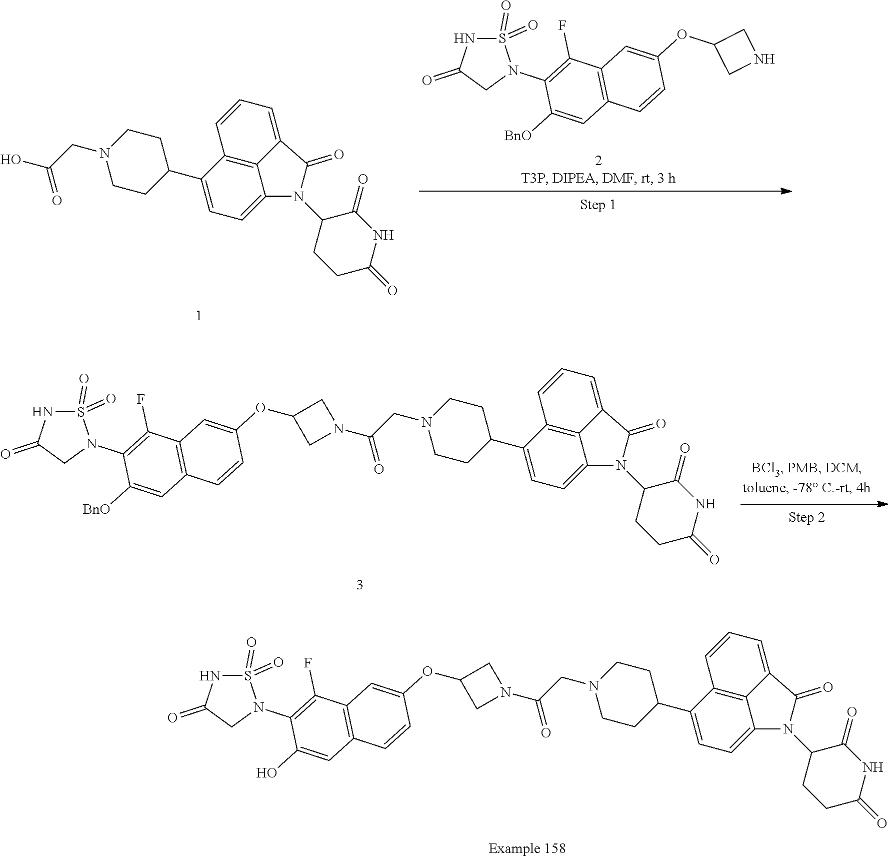 [SMILES: O=C(O)CN1CCC(c2ccc3c4c(cccc24)C(=O)N3C2CCC(=O)NC2=O)CC1 ; O=C1CN(c2c(OCc3ccccc3)cc3ccc(OC4CNC4)cc3c2F)S(=O)(=O)N1 ; O=C1CCC(N2C(=O)c3cccc4c(C5CCN(CC(=O)N6CC(Oc7ccc8cc(OCc9ccccc9)c(N9CC(=O)NS9(=O)=O)c(F)c8c7)C6)CC5)ccc2c34)C(=O)N1 ; O=C1CCC(N2C(=O)c3cccc4c(C5CCN(CC(=O)N6CC(Oc7ccc8cc(O)c(N9CC(=O)NS9(=O)=O)c(F)c8c7)C6)CC5)ccc2c34)C(=O)N1]

### Step 1: 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (3)

Into a 25 mL single neck round bottom flask containing a well-stirred solution of 2-[4-[1-(2,6-dioxo-3-piperidyl)-2-oxo-benzo[cd]indol-6-yl]-1-piperidyl]acetic acid (1, 150 mg, 255.87 μmol, TFA salt) in anhydrous DMF (2 mL) were added propylphosphonic anhydride (≥50 wt. % in ethyl acetate) (488.48 mg, 767.61 μmol, 0.45 mL) and DIPEA (296.80 mg, 2.30 mmol, 0.4 mL). After min, 5-[7-(azetidin-3-yloxy)-3-benzyloxy-1-fluoro-2-naphthyl]-1,1-dioxo-1,2,5-thiadiazolidin-3-one (2, 126.38 mg, 255.87 μmol, HCl salt) in anhydrous DMF (2 mL) was added. After 3 h, the solvent was removed and quenched with water (20 mL). The precipitate was filtered and dried under reduced pressure to afford 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (3, 180 mg, 150.25 μmol, 59% yield, 72% purity) as yellow solid. The material was used in the next step without further purification. LCMS (ES+): m/z 861.3 [M+H]⁺

### Step 2: 3-[6-[1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (Example 158)

Into a 50 mL single neck round bottom flask containing a well-stirred solution of 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (3, 180 mg, 150.25 μmol) and pentamethylbenzene (111.37 mg, 751.23 μmol) in anhydrous DCM (5 mL) and toluene (5 mL) was added boron trichloride (1.0 M in DCM) (352.09 mg, 3.00 mmol, 3 mL) at −78° C. After 4 h at RT, the reaction was quenched with 3 mL of 5% MeOH in DCM at −78° C. The volatiles were removed under reduced pressure and the residue was triturated with diethyl ether (50 mL) and purified by reverse phase prep HPLC [Purification method: Column: X-Select C18 (150×19) 5 micron; Mobile phase A: 0.1% Formic acid in water; Mobile phase B: MeCN] to afford 3-[6-[1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (Example 158, 60 mg, 73.15 μmol, 49% yield, Formic acid salt) as a yellow solid. LCMS (ES+): m/z 771.2 [M+H]⁺

¹H NMR (400 MHZ, DMSO-d₆) δ 11.13 (s, 1H), 9.82 (s, 1H), 8.45 (d, J=8.4 Hz, 1H), 8.10 (d, J=7.0 Hz, 1H), 7.90-7.83 (m, 1H), 7.72 (d, J=9.0 Hz, 1H), 7.37 (d, J=7.5 Hz, 1H), 7.17 (dd, J=9.0, 2.5 Hz, 1H), 7.12 (d, J=7.5 Hz, 1H), 7.05 (s, 1H), 6.98 (d, J=2.6 Hz, 1H), 5.41 (dd, J=12.7, 5.4 Hz, 1H), 5.27-5.19 (m, 1H), 4.81-4.71 (m, 1H), 4.47 (dd, J=10.8, 6.5 Hz, 1H), 4.25 (dd, J=9.9, 3.7 Hz, 1H), 4.18-4.06 (m, 2H), 3.98 (dd, J=10.3, 3.5 Hz, 2H), 3.39-3.32 (m, 3H), 3.01-2.84 (m, 3H), 2.81-2.61 (m, 2H), 2.14-1.90 (m, 5H).

### 2-[4-[1-(2,6-dioxo-3-piperidyl)-3-methyl-2-oxo-benzimidazol-5-yl]piperazin-1-yl]-N-[5-fluoro-7-hydroxy-6-(1,1,4-trioxo-1,2,5-thiadiazolidin-2-yl)-2-naphthyl]acetamide (Example 159)

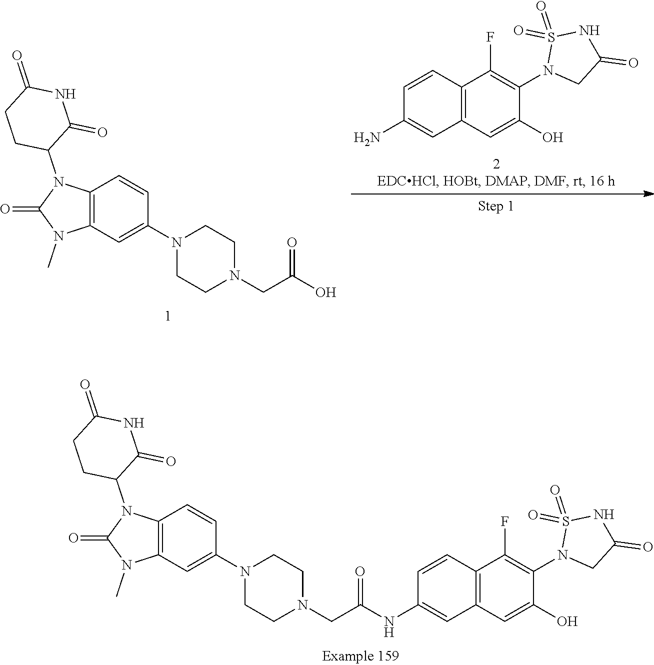 [SMILES: Cn1c(=O)n(C2CCC(=O)NC2=O)c2ccc(N3CCN(CC(=O)O)CC3)cc21 ; Nc1ccc2c(F)c(N3CC(=O)NS3(=O)=O)c(O)cc2c1 ; Cn1c(=O)n(C2CCC(=O)NC2=O)c2ccc(N3CCN(CC(=O)Nc4ccc5c(F)c(N6CC(=O)NS6(=O)=O)c(O)cc5c4)CC3)cc21]

### Step 1: 2-[4-[1-(2,6-dioxo-3-piperidyl)-3-methyl-2-oxo-benzimidazol-5-yl]piperazin-1-yl]-N-[5-fluoro-7-hydroxy-6-(1,1,4-trioxo-1,2,5-thiadiazolidin-2-yl)-2-naphthyl]acetamide (Example 159)

To a 50 mL single neck round bottom flask containing a solution of 2-[4-[1-(2,6-dioxo-3-piperidyl)-3-methyl-2-oxo-benzimidazol-5-yl]piperazin-1-yl]acetic acid (1, 500 mg, 679.03 μmol, TFA salt) in DMF (5 mL) were added 5-(6-amino-1-fluoro-3-hydroxy-2-naphthyl)-1,1-dioxo-1,2,5-thiadiazolidin-3-one (2, 196.78 mg, 475.32 μmol, HCl salt), HOBt (183.51 mg, 1.36 mmol), EDC HCl (260.34 mg, 1.36 mmol) and DMAP (414.78 mg, 3.4 mmol). After 16 h the reaction mixture was concentrated under reduced pressure and purified by reverse phase prep HPLC [Purification method: Column: X Select C18 (19×150 mm) 5 micron; Mobile phase A: 0.1% TFA in water; Mobile phase B: MeCN] to obtain 2-[4-[1-(2,6-dioxo-3-piperidyl)-3-methyl-2-oxo-benzimidazol-5-yl]piperazin-1-yl]-N-[5-fluoro-7-hydroxy-6-(1,1,4-trioxo-1,2,5-thiadiazolidin-2-yl)-2-naphthyl]acetamide (Example 159, 23 mg, 25.06 μmol, 4% yield, TFA salt) as a pale brown solid. LCMS (ES−): m/z 692.8 [M−H]⁻

¹H NMR (400 MHZ, DMSO-d₆) δ 11.08 (s, 1H), 10.82 (s, 1H), 10.09-9.93 (m, 1H), 8.14-8.08 (m, 1H), 7.91 (d, J=8.9 Hz, 1H), 7.48 (dd, J=9.1, 2.0 Hz, 1H), 7.04-6.98 (m, 2H), 6.96-6.90 (m, 1H), 6.71 (dd, J=8.6, 2.3 Hz, 1H), 5.31 (dd, J=12.9, 5.4 Hz, 1H), 4.33-4.23 (m, 2H), 4.17 (s, 2H), 3.33 (s, 3H), 2.96-2.84 (m, 1H), 2.78-2.57 (m, 2H), 2.05-1.93 (m, 1H). Note: additional peaks under solvent signal

### 2-[3-[1-(2,6-dioxo-3-piperidyl)-3-methyl-2-oxo-benzimidazol-5-yl]azetidin-1-yl]-N-[5-fluoro-7-hydroxy-6-(1,1,4-trioxo-1,2,5-thiadiazolidin-2-yl)-2-naphthyl]acetamide (Example 160)

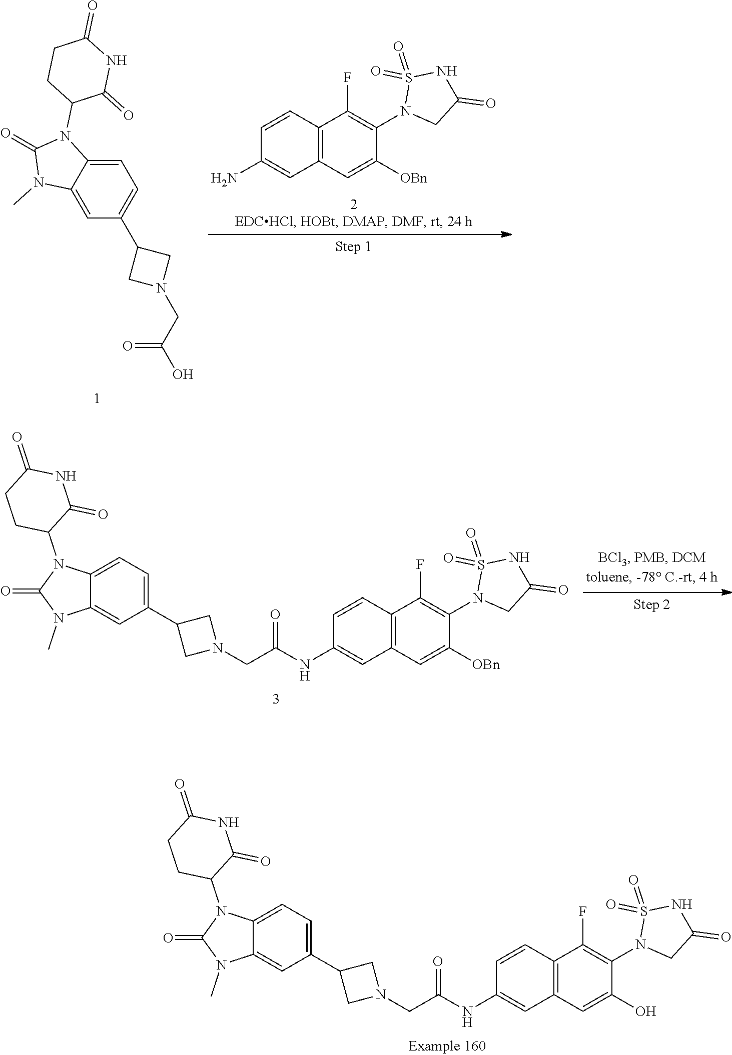 [SMILES: Cn1c(=O)n(C2CCC(=O)NC2=O)c2ccc(C3CN(CC(=O)O)C3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CN(CC(=O)Nc4ccc5c(F)c(N6CC(=O)NS6(=O)=O)c(OCc6ccccc6)cc5c4)C3)cc21 ; Cn1c(=O)n(C2CCC(=O)NC2=O)c2ccc(C3CN(CC(=O)Nc4ccc5c(F)c(N6CC(=O)NS6(=O)=O)c(O)cc5c4)C3)cc21]

### Step 1: N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]azetidin-1-yl]acetamide (3)

Into a 10 mL single neck round bottom flask containing a well-stirred solution of 2-[3-[1-(2,6-dioxo-3-piperidyl)-3-methyl-2-oxo-benzimidazol-5-yl]azetidin-1-yl]acetic acid (1, 150 mg, 351.34 μmol, Formic acid salt) and 5-(6-amino-3-benzyloxy-1-fluoro-2-naphthyl)-1,1-dioxo-1,2,5-thiadiazolidin-3-one (2, 196.84 mg, 351.34 μmol, TFA salt) in anhydrous DMF (5 mL) were added EDC HCl (202.06 mg, 1.05 mmol), DMAP (257.53 mg, 2.11 mmol) and HOBt (142.42 mg, 1.05 mmol). After 24 h, the solvent was removed, and the residue was treated with 1.5 N aqueous HCl (50 mL). The precipitate was filtered and dried under reduced pressure to afford N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]azetidin-1-yl]acetamide (3, 200 mg, 156.52 μmol, 45% yield, 62% purity, HCl salt) as off white solid. The material was used in the next step without further purification. LCMS (ES+): m/z 756.2 [M+H]⁺

### Step 2: 2-[3-[1-(2,6-dioxo-3-piperidyl)-3-methyl-2-oxo-benzimidazol-5-yl]azetidin-1-yl]-N-[5-fluoro-7-hydroxy-6-(1,1,4-trioxo-1,2,5-thiadiazolidin-2-yl)-2-naphthyl]acetamide (Example 160)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[3-[1-(2,6-dioxo-3-piperidyl)-3-methyl-2-oxo-benzimidazol-5-yl]azetidin-1-yl]acetamide (3, 200 mg, 156.52 μmol, HCl salt) and pentamethylbenzene (116.02 mg, 782.60 μmol) in anhydrous DCM (3 mL) and toluene (3 mL) was added boron trichloride (1.0 M in DCM) (366.79 mg, 3.13 mmol, 3.13 mL) at −78° C. After 4 h at RT, the reaction was quenched with 5% MeOH in DCM (3 mL) at −78° C. The volatiles were removed under reduced pressure and the residue was triturated with diethyl ether (50 mL) and purified by reverse phase prep HPLC [Purification method: Column: YMC C18 (250×20 mm, 5 micron; Mobile phase: 0.1% TFA in water; Mobile phase B: MeCN] to afford 2-[3-[1-(2,6-dioxo-3-piperidyl)-3-methyl-2-oxo-benzimidazol-5-yl]azetidin-1-yl]-N-[5-fluoro-7-hydroxy-6-(1,1,4-trioxo-1,2,5-thiadiazolidin-2-yl)-2-naphthyl]acetamide (Example 160, 60 mg, 75.89 μmol, 49% yield, TFA salt) as an off-white solid. LCMS (ES+): m/z 666.1 [M+H]⁺

¹H NMR (400 MHZ, DMSO-d₆) δ 11.11 (s, 1H), 10.87-10.73 (m, 1H), 8.10 (s, 1H), 7.90 (d, J=8.9 Hz, 1H), 7.48-7.41 (m, 1H), 7.37 (s, 1H), 7.18-7.08 (m, 2H), 7.00 (s, 1H), 5.37 (dd, J=12.7, 5.4 Hz, 1H), 4.62-4.47 (m, 3H), 4.46-4.38 (m, 2H), 4.38-4.29 (m, 2H), 4.22 (s, 2H), 3.37 (s, 3H), 2.98-2.82 (m, 1H), 2.79-2.58 (m, 2H), 2.06-1.96 (m, 1H).

### 2-[3-[1-(2,6-dioxo-3-piperidyl)-3-methyl-2-oxo-benzimidazol-5-yl]azetidin-1-yl]-N-[2-[[8-fluoro-6-hydroxy-7-(1,1,4-trioxo-1,2,5-thiadiazolidin-2-yl)-2-naphthyl]oxy]ethyl]acetamide (Example 161)

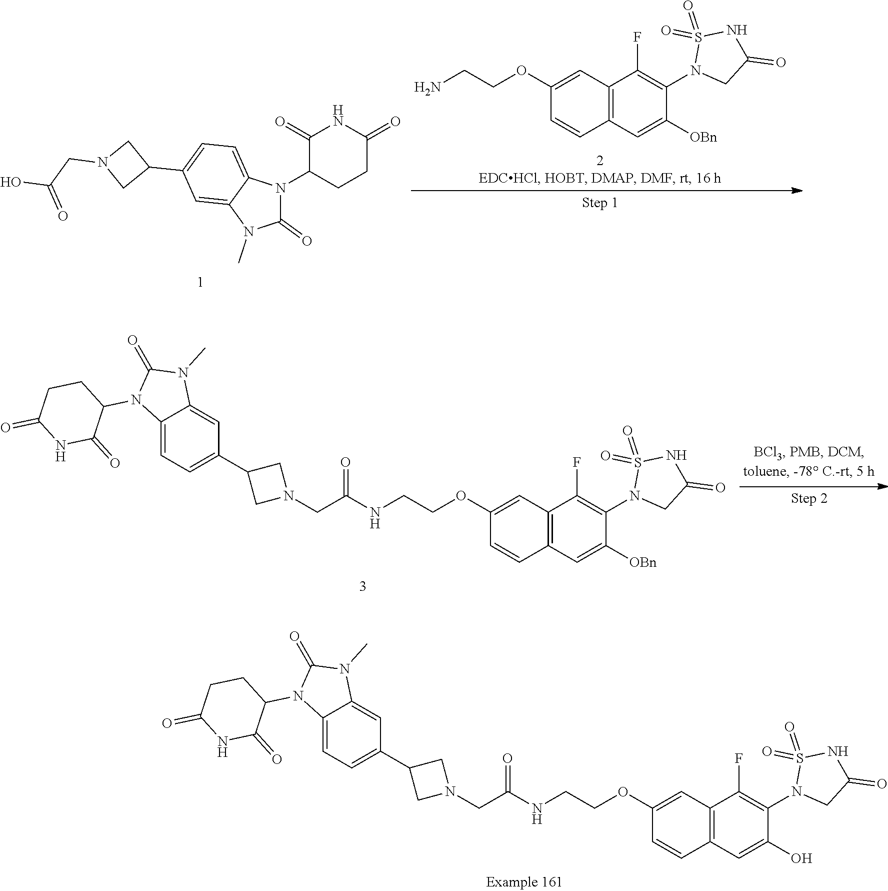 [SMILES: Cn1c(=O)n(C2CCC(=O)NC2=O)c2ccc(C3CN(CC(=O)O)C3)cc21 ; NCCOc1ccc2cc(OCc3ccccc3)c(N3CC(=O)NS3(=O)=O)c(F)c2c1 ; Cn1c(=O)n(C2CCC(=O)NC2=O)c2ccc(C3CN(CC(=O)NCCOc4ccc5cc(OCc6ccccc6)c(N6CC(=O)NS6(=O)=O)c(F)c5c4)C3)cc21 ; Cn1c(=O)n(C2CCC(=O)NC2=O)c2ccc(C3CN(CC(=O)NCCOc4ccc5cc(O)c(N6CC(=O)NS6(=O)=O)c(F)c5c4)C3)cc21]

### Step 1: N-(2-((6-(benzyloxy)-7-(1,1-dioxido-4-oxo-1,2,5-thiadiazolidin-2-yl)-8-fluoronaphthalen-2-yl)oxy)ethyl)-2-(3-(1-(2,6-dioxopiperidin-3-yl)-3-methyl-2-oxo-2,3-dihydro-1H-benzo[d]imidazol-5-yl)azetidin-1-yl)acetamide (3)

Into a 25 mL round bottom flask containing a well-stirred solution of 2-(3-(1-(2,6-dioxopiperidin-3-yl)-3-methyl-2-oxo-2,3-dihydro-1H-benzo[d]imidazol-5-yl)azetidin-1-yl)acetic acid (1, 151.85 mg, 308.39 μmol, TFA salt) and 5-(7-(2-aminoethoxy)-3-(benzyloxy)-1-fluoronaphthalen-2-yl)-1,2,5-thiadiazolidin-3-one 1,1-dioxide (2, 152.64 mg, 285.06 μmol, HCl salt) in anhydrous DMF (4 mL) were added EDC HCl (118.24 mg, 616.78 μmol) and DMAP (150.70 mg, 1.23 mmol). After 16 h, the solvent was removed under reduced pressure. The residue was suspended in 1.5 N aqueous HCl (50 mL) and the precipitate was filtered and to afford the N-(2-((6-(benzyloxy)-7-(1,1-dioxido-4-oxo-1,2,5-thiadiazolidin-2-yl)-8-fluoronaphthalen-2-yl)oxy)ethyl)-2-(3-(1-(2,6-dioxopiperidin-3-yl)-3-methyl-2-oxo-2,3-dihydro-1H-benzo[d]imidazol-5-yl)azetidin-1-yl)acetamide (3, 0.26 g, 233.17 μmol, 76% yield, 75% purity, HCl salt) as an off-white solid. The material was used in the next step without further purification. LCMS (ES+): m/z 800.3 [M+H]⁺

### Step 2: 2-[3-[1-(2,6-dioxo-3-piperidyl)-3-methyl-2-oxo-benzimidazol-5-yl]azetidin-1-yl]-N-[2-[[8-fluoro-6-hydroxy-7-(1,1,4-trioxo-1,2,5-thiadiazolidin-2-yl)-2-naphthyl]oxy]ethyl]acetamide (Example 161)

Into a 25 mL round bottom flask containing a well-stirred solution of N-(2-((6-(benzyloxy)-7-(1,1-dioxido-4-oxo-1,2,5-thiadiazolidin-2-yl)-8-fluoronaphthalen-2-yl)oxy)ethyl)-2-(3-(1-(2,6-dioxopiperidin-3-yl)-3-methyl-2-oxo-2,3-dihydro-1H-benzo[d]imidazol-5-yl)azetidin-1-yl)acetamide (3, 0.26 g, 233.17 μmol. HCl salt) and pentamethylbenzene (172.84 mg, 1.17 mmol) in a mixture of DCM (1 mL) and toluene (1 mL) was added a boron trichloride (1.0 M in DCM) (819.63 mg, 7.00 mmol) at −78° C. The reaction mixture was stirred at RT for 5 h. The reaction mixture was cooled to −78° C. and quenched by dropwise addition of 10% MeOH in DCM (1.2 mL). The volatiles were removed under reduced pressure and the residue was triturated by diethyl ether (16 mL), filtered and dried. The material was purified by reverse phase prep HPLC [Purification method: Column: X-Bridge C18 (150)×10) 5 micron; Mobile phase A: 0.1% TFA in water; Mobile phase B: MeCN] to afford 2-[3-[1-(2,6-dioxo-3-piperidyl)-3-methyl-2-oxo-benzimidazol-5-yl]azetidin-1-yl]-N-[2-[[8-fluoro-6-hydroxy-7-(1,1,4-trioxo-1,2,5-thiadiazolidin-2-yl)-2-naphthyl]oxy]ethyl]acetamide (Example 161, 37 mg, 44.33 μmol, 19% yield, TFA salt) as a white solid. LCMS (ES+): m/z 710.2 [M+H]⁺

¹H NMR (400 MHZ, DMSO-d₆) δ 11.10 (d, J=3.6 Hz, 1H), 10.37 (s, 1H), 9.60 (s, 1H), 8.83-8.70 (m, 1H), 7.70 (d, J=9.0 Hz, 1H), 7.34 (s, 1H), 7.22 (d, J=2.5 Hz, 1H), 7.17-7.06 (m, 3H), 7.04 (s, 1H), 5.36 (dd, J=12.7, 5.5 Hz, 1H), 4.51-4.40) (m, 1H), 4.39-4.32 (m, 1H), 4.29-4.14 (m, 5H), 4.14 (s, 3H), 3.61-3.54 (m, 2H), 3.36 (d, J=5.5 Hz, 4H), 2.97-2.83 (m, 1H), 2.79-2.57 (m, 2H), 2.04-1.94 (m, 1H).

### 3-[5-[3,3-difluoro-1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 162)

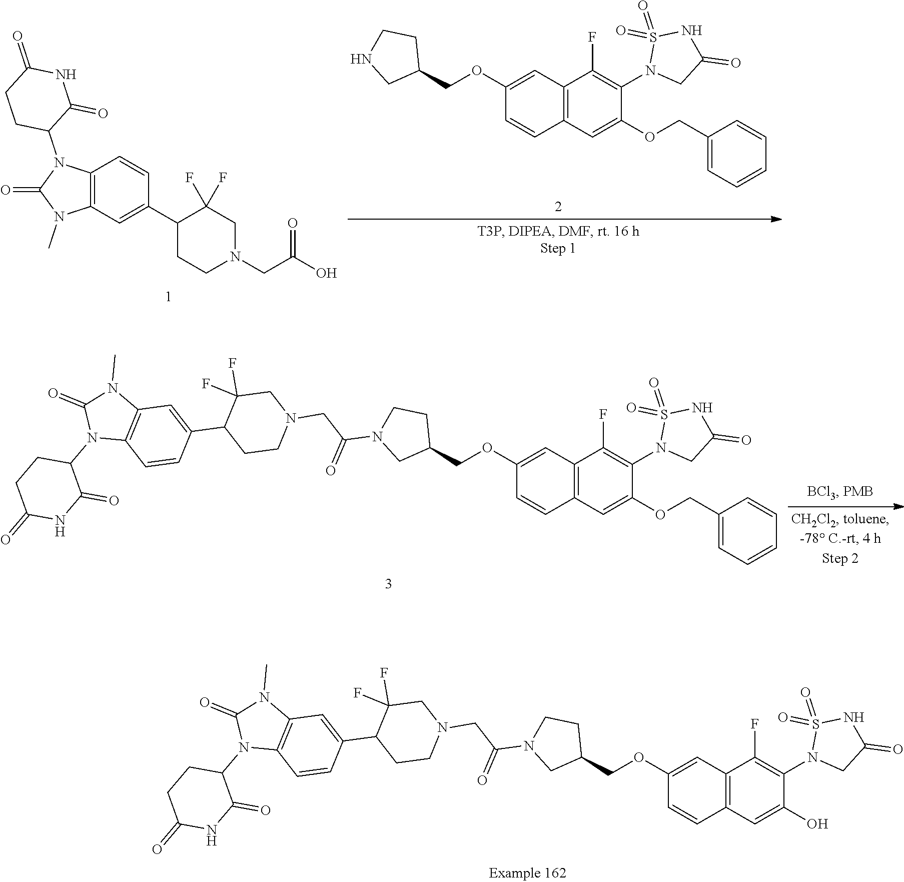 [SMILES: Cn1c(=O)n(C2CCC(=O)NC2=O)c2ccc(C3CCN(CC(=O)O)CC3(F)F)cc21 ; O=C1CN(c2c(OCc3ccccc3)cc3ccc(OC[C@@H]4CCNC4)cc3c2F)S(=O)(=O)N1 ; Cn1c(=O)n(C2CCC(=O)NC2=O)c2ccc(C3CCN(CC(=O)N4CC[C@@H](COc5ccc6cc(OCc7ccccc7)c(N7CC(=O)NS7(=O)=O)c(F)c6c5)C4)CC3(F)F)cc21 ; Cn1c(=O)n(C2CCC(=O)NC2=O)c2ccc(C3CCN(CC(=O)N4CC[C@@H](COc5ccc6cc(O)c(N7CC(=O)NS7(=O)=O)c(F)c6c5)C4)CC3(F)F)cc21]

### Step 1: 3-[5-[1-[2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]-2-oxo-ethyl]-3,3-difluoro-4-piperidyl]-3-methyl-2-oxo-benzimidazol-1-yl]piperidine-2,6-dione (3)

Into a 50 mL single neck, round bottom flask containing a well-stirred solution of 2-[4-[1-(2,6-dioxo-3-piperidyl)-3-methyl-2-oxo-benzimidazol-5-yl]-3,3-difluoro-1-piperidyl]acetic acid (1, 150 mg, 234.33 μmol, TFA salt) in DMF (2.0 mL) was added propylphosphonic anhydride solution (50 Wt. % in EtOAc) (223.6 mg, 351.50 μmol) followed by DIPEA (90.86 mg, 703.00 μmol, 122.45 μL). After 2 h, 5-[3-benzyloxy-1-fluoro-7-[[(3R)-pyrrolidin-3-yl]methoxy]-2-naphthyl]-1,1-dioxo-1,2,5-thiadiazolidin-3-one (2, 113.78 mg, 217.97 μmol, HCl salt) was added. After 16 h, the reaction mixture was concentrated under reduced pressure and triturated with Et₂O to afford 3-[5-[1-[2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]-2-oxo-ethyl]-3,3-difluoro-4-piperidyl]-3-methyl-2-oxo-benzimidazol-1-yl]piperidine-2,6-dione (3, 230 mg, 142.58 μmol, 61% yield, 56% purity) as an off-white solid. LCMS (ES+): m/z 904.2 [M+H]⁺

¹H NMR (400 MHZ, DMSO-d₆): δ 11.12 (s, 1H), 10.05 (s, 1H), 7.73-7.71 (m, 1H), 7.25-7.00 (m, 6H), 5.38 (dd, J=5.20, 12.40 Hz, 1H), 4.32 (s, 2H), 4.00-3.80 (m, 4H), 4.20-4.08 (m, 5H), 3.25 (s, 6H), 2.95-2.62 (m, 5H), 2.35-1.75 (m, 5H).

### Step 2: 3-[5-[3,3-difluoro-1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 162)

Into a 25 mL single neck, round bottom flask containing a well-stirred solution of 3-[5-[1-[2-[(3R)-3-[[6-benzyloxy-8-fluoro-7-(1,1,4-trioxo-1,2,5-thiadiazolidin-2-yl)-2-naphthyl]oxymethyl]pyrrolidin-1-yl]-2-oxo-ethyl]-3,3-difluoro-4-piperidyl]-3-methyl-2-oxo-benzimidazol-1-yl]piperidine-2,6-dione (3, 200 mg, 188.07 μmol) in anhydrous DCM (4 mL) and anhydrous toluene (3.5 mL) was added pentamethylbenzene (139.40 mg, 940.35 μmol) under nitrogen atmosphere. The reaction mixture was cooled to −78° C. and treated with BCl₃ (1.0 M in DCM) (1.88 mmol, 1.9 mL) via dropwise addition. The resulting mixture was stirred at RT for 3 h. The reaction mixture was cooled to −78° C. and quenched with 10% MeOH in DCM (5 mL) and concentrated under reduced pressure and purified by reverse-phase preparative HPLC (X-BRIDGE C18 (19×150 mm) 5.0 μm; Mobile Phase A: 0.1% TFA in water and Mobile Phase B: CH₃CN) to afford 3-[5-[3,3-difluoro-1-[2-[(3R)-3-[[8-fluoro-6-hydroxy-7-(1,1,4-trioxo-1,2,5-thiadiazolidin-2-yl)-2-naphthyl]oxymethyl]pyrrolidin-1-yl]-2-oxo-ethyl]-4-piperidyl]-3-methyl-2-oxo-benzimidazol-1-yl]piperidine-2,6-dione (Example 162, 40 mg, 40.97 μmol, 25% yield, TFA salt) as an off-white solid. LCMS (ES+): m/z 814.2 [M+H]⁺

¹H NMR (400 MHZ, DMSO-d₆) δ 11.11 (s, 1H), 10.00 (s, 1H), 7.75-7.65 (m, 1H), 7.26-7.21 (m, 1H), 7.18 (dt, J=9.0, 2.5 Hz, 1H), 7.14-7.08 (m, 2H), 7.06 (s, 1H), 7.01-6.96 (m, 1H), 5.37 (dd, J=12.8, 5.4 Hz, 1H), 4.31 (d, J=1.6 Hz, 2H), 4.22-4.01 (m, 4H), 3.92-3.77 (m, 2H), 3.35 (s, 3H), 3.29-3.17 (m, 2H), 2.98-2.78 (m, 2H), 2.77-2.58 (m, 3H), 2.24-1.98 (m, 3H), 1.96-1.87 (m, 1H), 1.86-1.74 (m, 1H).

### 2-[1-[3-[(3S)-2,6-dioxo-3-piperidyl]-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 163)

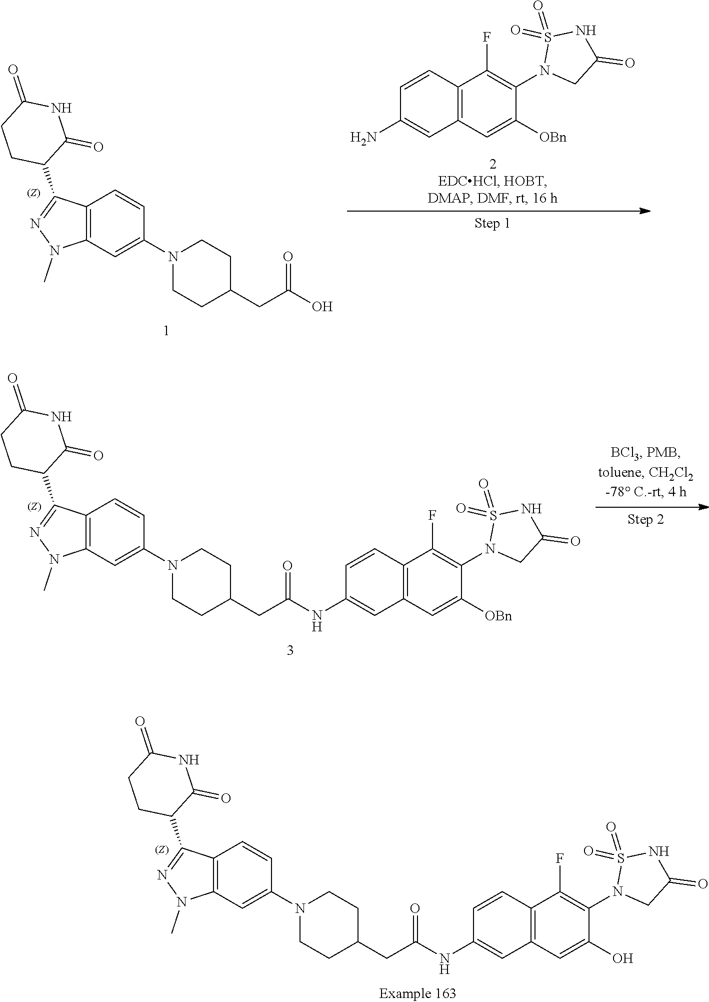

### Step 1: N-[7-benzyloxy-5-fluoro-6-(1,1,4-trioxo-1,2,5-thiadiazolidin-2-yl)-2-naphthyl]-2-[1-[3-[(3S)-2,6-dioxo-3-piperidyl]-1-methyl-indazol-6-yl]-4-piperidyl]acetamide (3)

To a 25 mL single neck round bottom flask containing a solution of (S)-2-(1-(3-(2,6-dioxopiperidin-3-yl)-1-methyl-1H-indazol-6-yl)piperidin-4-yl)acetic acid (1, 90 mg, 234.11 μmol) in DMF (2.5 mL) were added 5-(6-amino-3-benzyloxy-1-fluoro-2-naphthyl)-1,1-dioxo-1,2,5-thiadiazolidin-3-one (2, 119.41 mg, 222.41 μmol, TFA salt), DMAP (57.20 mg, 468.23 μmol). HOBt (63.27 mg, 468.23 μmol) and EDC (89.76 mg, 468.23 μmol). After 16 h, the solvent was removed under reduced pressure and the residue treated with aq. 1.5 N HCl (10 mL). The precipitate was collected by filtration and dried under vacuum to obtain N-[7-benzyloxy-5-fluoro-6-(1,1,4-trioxo-1,2,5-thiadiazolidin-2-yl)-2-naphthyl]-2-[1-[3-[(3S)-2,6-dioxo-3-piperidyl]-1-methyl-indazol-6-yl]-4-piperidyl]acetamide (3, 162 mg, 170.90 μmol, 73% yield) as a pale yellow solid. LCMS (ES+): m/z 768.2 [M+H]⁺

### Step 2: 2-[1-[3-[(3S)-2,6-dioxo-3-piperidyl]-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 163)

To a 50 mL single neck round bottom flask, containing a well-stirred suspension of N-[7-benzyloxy-5-fluoro-6-(1,1,4-trioxo-1,2,5-thiadiazolidin-2-yl)-2-naphthyl]-2-[1-[3-[(3S)-2,6-dioxo-3-piperidyl]-1-methyl-indazol-6-yl]-4-piperidyl]acetamide (3, 160 mg, 168.79 μmol) in a mixture of toluene (1.5 mL) and DCM (1.5 mL) was added pentamethylbenzene (125.11 mg, 843.94 μmol) under nitrogen atmosphere. The resulting suspension was cooled to −78° C. and treated with boron trichloride (1.0 M in DCM) (395.54 mg, 3.38 mmol, 3.38 mL) via dropwise addition. The reaction mixture was stirred at ambient temperature for 4 h. The reaction mixture was cooled to −78° C. and quenched with 5% MeOH in DCM (2 mL). The solvent was removed under reduced pressure, the residue was triturated with diethyl ether (2×20 mL), filtered and dried. The material was purified by reverse phase prep HPLC [Purification method: Column: X-Select C18 (30×150 mm), 5 Micron; Mobile Phase: 0.1% TFA in water; Mobile phase B: MeCN] to obtain 2-[1-[3-[(3S)-2,6-dioxo-3-piperidyl]-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 163, 56 mg, 68.40 μmol, 41% yield, TFA salt) as a pale brown solid. LCMS (ES+): m/z 678.3 [M+H]^{+ 1}H NMR (400 MHZ, DMSO-d₆) δ 10.89 (s, 1H), 10.37 (d, J=30.6 Hz, 1H), 10.21 (s, 1H), 8.20 (d, J=1.9 Hz, 1H), 7.86 (d, J=9.0 Hz, 1H), 7.60 (d, J=9.0 Hz, 1H), 7.44 (dd, J=9.1, 2.0 Hz, 1H), 7.23-7.10 (m, 1H), 7.05 (d, J=8.8 Hz, 1H), 6.97 (s, 1H), 4.39 (s, 2H), 4.30 (dd, J=9.6, 5.1 Hz, 1H), 3.93 (s, 3H), 3.77 (d, J=12.1 Hz, 2H), 3.03 (s, 2H), 2.71-2.55 (m, 2H), 2.42-2.27 (m, 3H), 2.20-2.02 (m, 2H), 1.94-1.85 (m, 2H), 1.59-1.43 (m, 2H).

### 2-[1-[3-[(3R)-2,6-dioxo-3-piperidyl]-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 164)

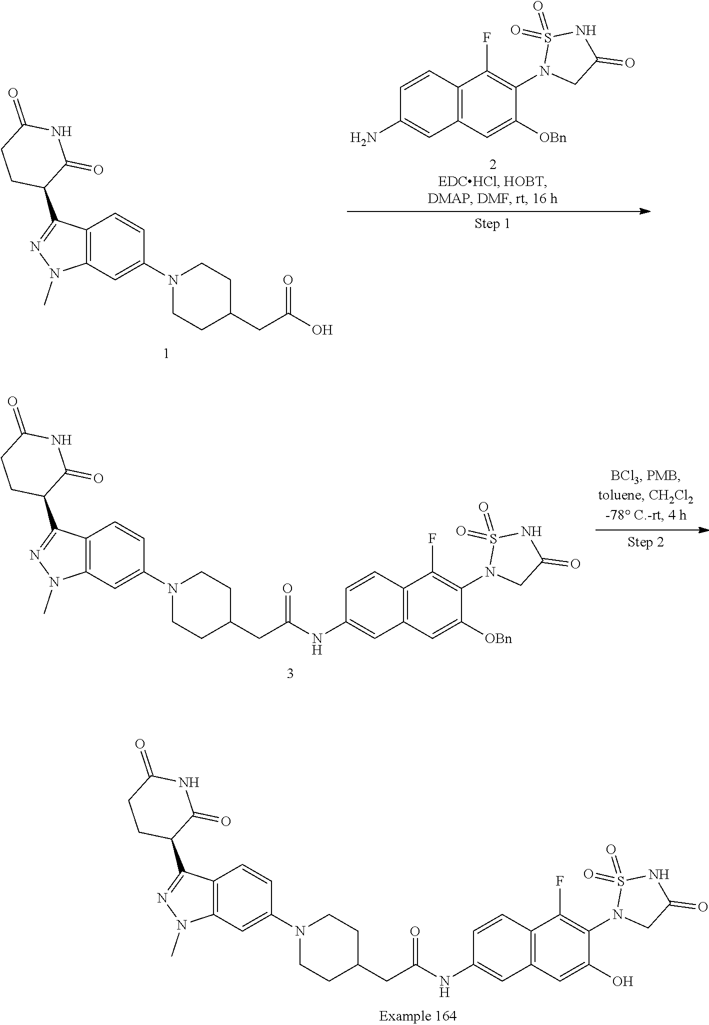 [SMILES: Cn1nc([C@H]2CCC(=O)NC2=O)c2ccc(N3CCC(CC(=O)O)CC3)cc21 ; Nc1ccc2c(F)c(N3CC(=O)NS3(=O)=O)c(OCc3ccccc3)cc2c1 ; Cn1nc([C@H]2CCC(=O)NC2=O)c2ccc(N3CCC(CC(=O)Nc4ccc5c(F)c(N6CC(=O)NS6(=O)=O)c(OCc6ccccc6)cc5c4)CC3)cc21 ; Cn1nc([C@H]2CCC(=O)NC2=O)c2ccc(N3CCC(CC(=O)Nc4ccc5c(F)c(N6CC(=O)NS6(=O)=O)c(O)cc5c4)CC3)cc21]

### 2-[1-[3-[(3R)-2,6-dioxo-3-piperidyl]-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 164)

2-[1-[3-[(3R)-2,6-dioxo-3-piperidyl]-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 164, 65 mg, 79.06 μmol, 53% yield) was prepared from (R)-2-(1-(3-(2,6-dioxopiperidin-3-yl)-1-methyl-1H-indazol-6-yl)piperidin-4-yl)acetic acid (1) and 5-(6-amino-3-benzyloxy-1-fluoro-2-naphthyl)-1,1-dioxo-1,2,5-thiadiazolidin-3-one (2) following the same procedure used for 2-[1-[3-[(3S)-2,6-dioxo-3-piperidyl]-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 163).

¹H NMR (400 MHZ, DMSO-d₆) δ 10.87 (s, 1H), 10.22-10.06 (m, 2H), 8.19 (d, J=2.1 Hz, 1H), 7.84 (d, J=8.9 Hz, 1H), 7.54 (d, J=8.7 Hz, 1H), 7.43 (dd, J=9.1, 2.0 Hz, 1H), 7.03-6.90 (m, 3H), 4.31-4.23 (m, 3H), 3.90 (s, 3H), 3.82-3.75 (m, 2H), 2.96-2.81 (m, 2H), 2.62 (q, J=7.7, 6.1 Hz, 1H), 2.42-2.25 (m, 3H), 2.21-2.10 (m, 1H), 2.10-1.96 (m, 1H), 1.85 (d, J=12.8 Hz, 2H), 1.52-1.36 (m, 2H).

### 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (Example 165)

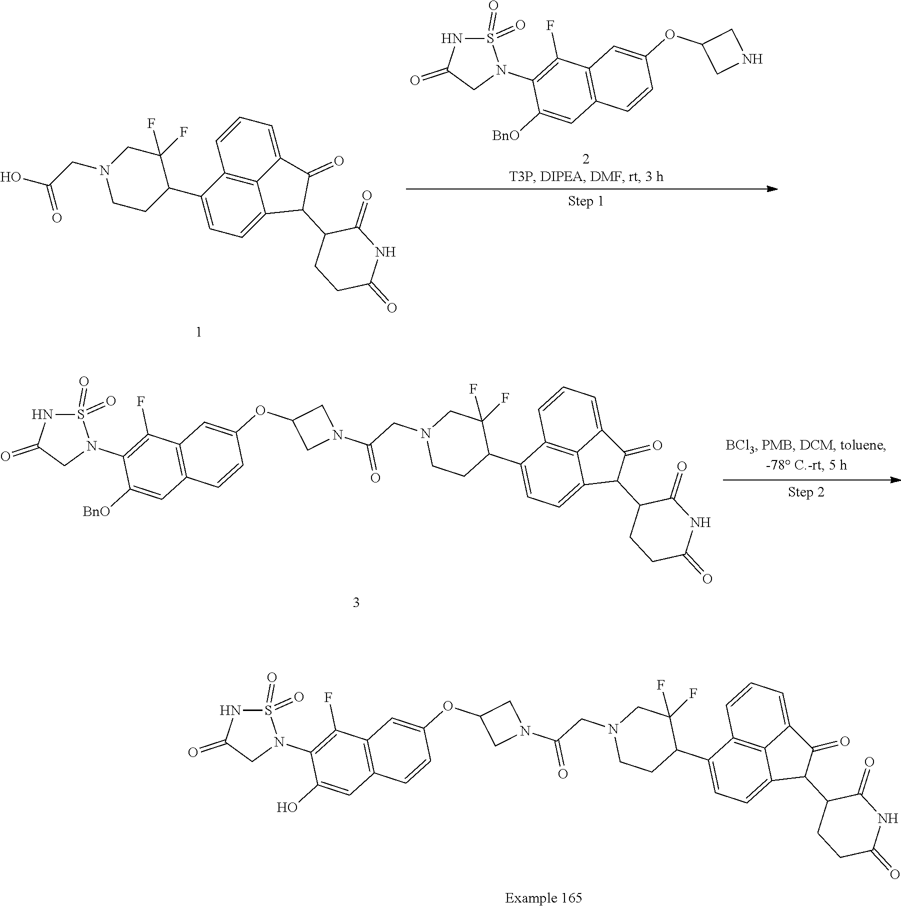 [SMILES: O=C(O)CN1CCC(c2ccc3c4c(cccc24)C(=O)C3C2CCC(=O)NC2=O)C(F)(F)C1 ; O=C1CN(c2c(OCc3ccccc3)cc3ccc(OC4CNC4)cc3c2F)S(=O)(=O)N1 ; O=C1CCC(C2C(=O)c3cccc4c(C5CCN(CC(=O)N6CC(Oc7ccc8cc(OCc9ccccc9)c(N9CC(=O)NS9(=O)=O)c(F)c8c7)C6)CC5(F)F)ccc2c34)C(=O)N1 ; O=C1CCC(C2C(=O)c3cccc4c(C5CCN(CC(=O)N6CC(Oc7ccc8cc(O)c(N9CC(=O)NS9(=O)=O)c(F)c8c7)C6)CC5(F)F)ccc2c34)C(=O)N1]

### Step 1: 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-2-oxo-benzo[cd]indol-1-yl]piperidine-2,6-dione (3)

Into a 25 mL single neck round bottom flask containing a well-stirred solution of 2-[4-[1-(2,6-dioxo-3-piperidyl)-2-oxo-benzo[cd]indol-6-yl]-3,3-difluoro-1-piperidyl]acetic acid (1, 180 mg, 245.69 μmol, TFA salt) in DMF (3 mL) was added DIPEA (158.77 mg, 1.23 mmol, 213.97 μL) and propylphosphonic anhydride (≥50 wt. % in ethyl acetate) (312.72 mg, 491.38 μmol, 0.3 mL, 50% purity). After 10 min, 5-[7-(azetidin-3-yloxy)-3-benzyloxy-1-fluoro-2-naphthyl]-1,1-dioxo-1,2,5-thiadiazolidin-3-one (2, 121.35 mg, 245.69 μmol, HCl salt) was added. After 3 h, the reaction mixture was concentrated under reduced pressure and the residue was treated with cold water (3 mL). The precipitate was filtered, washed with cold water (5 mL) and diethyl ether (5 mL) to afford 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-2-oxo-benzo[cd]indol-1-yl]piperidine-2,6-dione (3, 150 mg, 87.54 μmol, 36% yield, 52% purity) as a pale yellow solid. The material was used in the next step without further purification. LCMS (ES+): m/z 896.7 [M+H]⁺

### Step 2: 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (Example 165)

Into a 50 mL single neck round bottom flask containing a well-stirred solution of 3-[6-[1-[2-[3-[[6-benzyloxy-8-fluoro-7-(1,1,4-trioxo-1,2,5-thiadiazolidin-2-yl)-2-naphthyl]oxy]azetidin-1-yl]-2-oxo-ethyl]-3,3-difluoro-4-piperidyl]-2-oxo-benzo[cd]indol-1-yl]piperidine-2,6-dione (3, 150 mg, 86.97 μmol) in DCM (4 mL) and toluene (4 mL) was added pentamethylbenzene (64.46 mg, 434.84 μmol, 70.30 μL) at RT. Then, boron trichloride (1.0 M in DCM) (203.23 mg, 1.74 mmol, 1.74 mL) was added at −75° C. The reaction mixture was stirred at RT for 5 h. The reaction mixture was quenched with 5% MeOH in DCM (2 mL) at −75° C. The reaction mixture was concentrated under reduced pressure, the residue was triturated with diethyl ether (10 mL), and purified by reverse phase prep HPLC [Purification method: Column: Xbridge C-18 (19×150 mm), 5 micron; Mobile phase A: 0.1% TFA in water; Mobile phase B: MeCN] to afford 3-[6-[3,3-difluoro-1-[2-[3-[[8-fluoro-6-hydroxy-7-(1,1,4-trioxo-1,2,5-thiadiazolidin-2-yl)-2-naphthyl]oxy]azetidin-1-yl]-2-oxo-ethyl]-4-piperidyl]-2-oxo-benzo[cd]indol-1-yl]piperidine-2,6-dione (Example 165, 53 mg, 54.80 μmol, 63% yield, TFA salt) as an off white solid. LCMS (ES+): m/z 806.8 [M+H]⁺

¹H NMR (400 MHZ, DMSO-d₆) δ 11.14 (d, J=2.9 Hz, 1H), 10.00 (s, 1H), 8.46 (d, J=8.4 Hz, 1H), 8.13 (d, J=6.9 Hz, 1H), 7.88 (dd, J=8.4, 7.0 Hz, 1H), 7.80-7.71 (m, 1H), 7.56-7.49 (m, 1H), 7.24-7.16 (m, 2H), 7.11-7.06 (m, 1H), 7.03-7.00 (m, 1H), 5.53-5.43 (m, 1H), 5.32-5.20 (m, 1H), 4.83-4.73 (m, 1H), 4.53-4.41 (m, 1H), 4.32-4.20 (m, 3H), 3.97 (d, J=10.7 Hz, 2H), 3.26 (s, 1H), 3.02-2.87 (m, 2H), 2.83-2.71 (m, OH), 2.65-2.54 (m, 1H), 2.15-2.05 (m, 1H), 2.05-1.89 (m, 1H). Note: additional peaks under solvent signal

### N-(2-((7-(1,1-dioxido-4-oxo-1,2,5-thiadiazolidin-2-yl)-8-fluoro-6-hydroxynaphthalen-2-yl)oxy)ethyl)-2-((3-(1-(2,6-dioxopiperidin-3-yl)-2-oxo-1,2-dihydrobenzo[cd]indol-6-yl)propyl)amino)-N-methylacetamide (Example 166)

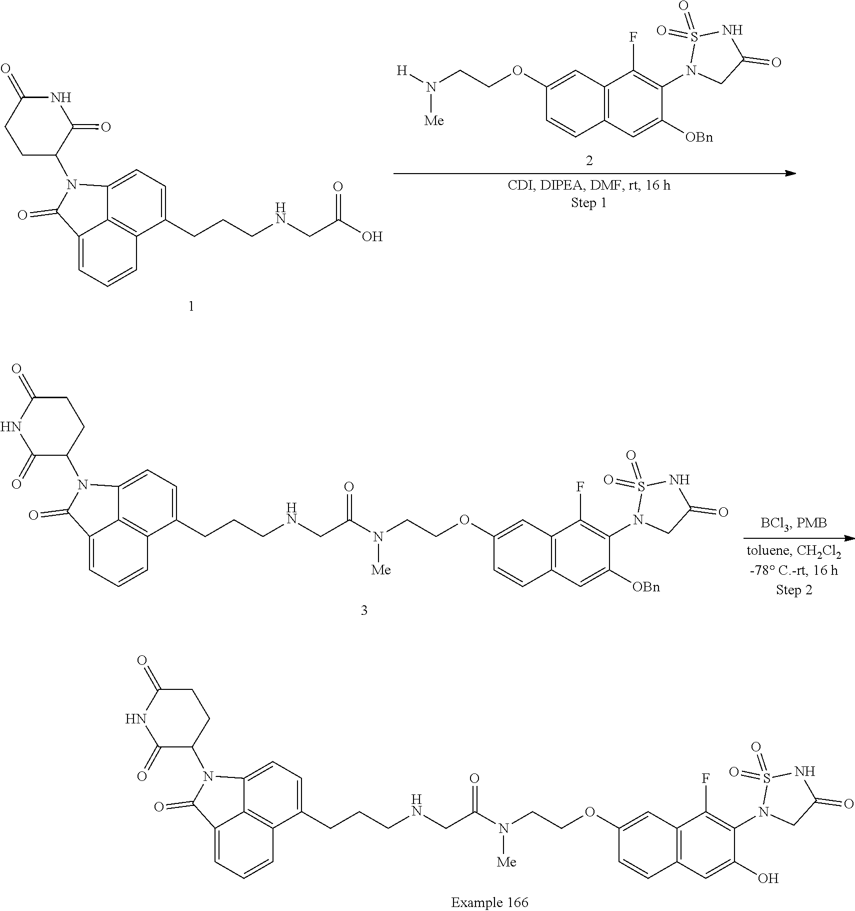 [SMILES: O=C(O)CNCCCc1ccc2c3c(cccc13)C(=O)N2C1CCC(=O)NC1=O ; CNCCOc1ccc2cc(OCc3ccccc3)c(N3CC(=O)NS3(=O)=O)c(F)c2c1 ; CN(CCOc1ccc2cc(OCc3ccccc3)c(N3CC(=O)NS3(=O)=O)c(F)c2c1)C(=O)CNCCCc1ccc2c3c(cccc13)C(=O)N2C1CCC(=O)NC1=O ; CN(CCOc1ccc2cc(O)c(N3CC(=O)NS3(=O)=O)c(F)c2c1)C(=O)CNCCCc1ccc2c3c(cccc13)C(=O)N2C1CCC(=O)NC1=O]

### Step 1: N-(2-((6-(benzyloxy)-7-(1,1-dioxido-4-oxo-1,2,5-thiadiazolidin-2-yl)-8-fluoronaphthalen-2-yl)oxy)ethyl)-2-((3-(1-(2,6-dioxopiperidin-3-yl)-2-oxo-1,2-dihydrobenzo[cd]indol-6-yl)propyl)amino)-N-methylacetamide (3)

Into a 50 mL single neck, round bottom flask containing a well-stirred solution of (3-(1-(2,6-dioxopiperidin-3-yl)-2-oxo-1,2-dihydrobenzo[cd]indol-6-yl)propyl)glycine (1, 170 mg, 273.64 μmol, TFA salt) in dry DMF (4 mL) were added CDI (133.11 mg, 820.92 μmol) and DIPEA (176.83 mg, 1.37 mmol, 238.31 μL). After 1 h, 5-[3-benzyloxy-1-fluoro-7-[2-(methylamino)ethoxy]-2-naphthyl]-1,1-dioxo-1,2,5-thiadiazolidin-3-one (2, 142.67 mg, 246.27 μmol, TFA salt). After 15 h, the reaction mixture was concentrated under reduced pressure and poured into 10 mL of water. The precipitate was filtered, washed with water and dried under vacuum to afford N-(2-((6-(benzyloxy)-7-(1,1-dioxido-4-oxo-1,2,5-thiadiazolidin-2-yl)-8-fluoronaphthalen-2-yl)oxy)ethyl)-2-((3-(1-(2,6-dioxopiperidin-3-yl)-2-oxo-1,2-dihydrobenzo[cd]indol-6-yl)propyl)amino)-N-methylacetamide (3, 150 mg, 71.52 μmol, 26% yield, 40% purity) as yellow solid. LCMS (ES+): m/z 837.2 [M+H]⁺

### Step 2: N-(2-((7-(1,1-dioxido-4-oxo-1,2,5-thiadiazolidin-2-yl)-8-fluoro-6-hydroxynaphthalen-2-yl)oxy)ethyl)-2-((3-(1-(2,6-dioxopiperidin-3-yl)-2-oxo-1,2-dihydrobenzo[cd]indol-6-yl)propyl)amino)-N-methylacetamide (Example 166)

Into a 100 mL single neck round bottom flask containing a well-stirred solution of N-(2-((6-(benzyloxy)-7-(1,1-dioxido-4-oxo-1,2,5-thiadiazolidin-2-yl)-8-fluoronaphthalen-2-yl)oxy)ethyl)-2-((3-(1-(2,6-dioxopiperidin-3-yl)-2-oxo-1,2-dihydrobenzo[cd]indol-6-yl)propyl)amino)-N-methylacetamide (3, 130 mg, 62.14 μmol) and pentamethylbenzene (46.06 mg, 310.68 μmol) in anhydrous DCM (4 mL) and toluene (4 mL) was added BCl₃ (1.0 M solution in DCM) (1.24 mmol, 1.24 mL) at −78° C. The reaction mixture was stirred at ambient temperature for 16 h. The reaction mixture was quenched with 3 mL of 5% MeOH in DCM at −78° C. and concentrated under reduced pressure to afford the crude material that was purified by reverse-phase preparative HPLC [Column: X-SELECT-C18 (150×30) mm, 5 microns; Mobile phase A: 0.1% formic acid in water and Mobile Phase B: CH₃CN] to afford N-(2-((7-(1,1-dioxido-4-oxo-1,2,5-thiadiazolidin-2-yl)-8-fluoro-6-hydroxynaphthalen-2-yl)oxy)ethyl)-2-((3-(1-(2,6-dioxopiperidin-3-yl)-2-oxo-1,2-dihydrobenzo[cd]indol-6-yl)propyl)amino)-N-methylacetamide (Example 166, 24 mg, 29.42 μmol, 47% yield, Formic acid salt) as pale yellow solid. LCMS (ES+): m/z 747.8 [M+H]⁺

¹H NMR (400 MHZ, DMSO-d₆) δ 11.12 (s, 1H), 9.48 (s, 1H), 8.38 (dd, J=8.3, 3.7 Hz, 1H), 8.15-8.08 (m, 1H), 7.87 (dd, J=8.2, 7.0 Hz, 1H), 7.70-7.64 (m, 1H), 7.35 (d, J=7.3 Hz, 1H), 7.24-7.19 (m, 1H), 7.16-7.06 (m, 2H), 7.03 (d, J=3.9 Hz, 1H), 5.44 (dd, J=12.9, 5.3 Hz, 1H), 4.24 (dt, J=20.5, 5.4 Hz, 2H), 4.08 (d, J=2.6 Hz, 3H), 4.00 (s, 1H), 3.81-3.69 (m, 2H), 3.14-3.07 (m, 2H), 3.06 (s, 1H), 3.01-2.90 (m, 4H), 2.82-2.71 (m, 1H), 2.65-2.56 (m, 1H), 2.13-1.96 (m, 3H).

### 2-[1-[3-(2,6-dioxo-3-piperidyl)-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 167)

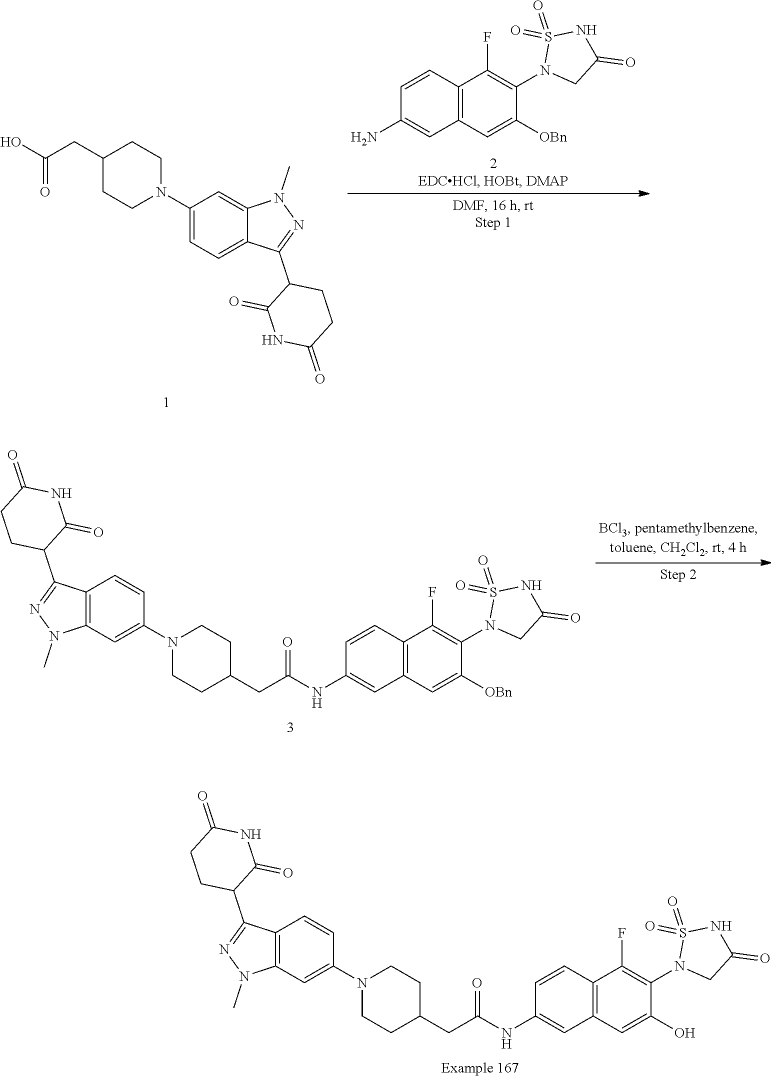 [SMILES: Nc1ccc2c(F)c(N3CC(=O)NS3(=O)=O)c(OCc3ccccc3)cc2c1 ; Cn1nc(C2CCC(=O)NC2=O)c2ccc(N3CCC(CC(=O)O)CC3)cc21 ; Cn1nc(C2CCC(=O)NC2=O)c2ccc(N3CCC(CC(=O)Nc4ccc5c(F)c(N6CC(=O)NS6(=O)=O)c(OCc6ccccc6)cc5c4)CC3)cc21 ; Cn1nc(C2CCC(=O)NC2=O)c2ccc(N3CCC(CC(=O)Nc4ccc5c(F)c(N6CC(=O)NS6(=O)=O)c(O)cc5c4)CC3)cc21]

### Step 1: N-[7-benzyloxy-5-fluoro-6-(1,1,4-trioxo-1,2,5-thiadiazolidin-2-yl)-2-naphthyl]-2-[1-[3-(2,6-dioxo-3-piperidyl)-1-methyl-indazol-6-yl]-4-piperidyl]acetamide (3)

To a stirred solution of 2-[1-[3-(2,6-dioxo-3-piperidyl)-1-methyl-indazol-6-yl]-4-piperidyl]acetic acid (1, 200 mg, 445.60 μmol) in DMF (2 mL) were added HOBt (180.63 mg, 1.34 mmol), DMAP (326.63 mg, 2.67 mmol) and 3-(ethyliminomethyleneamino)-N,N-dimethyl-propan-1-amine; hydrochloride (170.84 mg, 891.19 μmol) at 0° C. 5-(6-amino-3-benzyloxy-1-fluoro-2-naphthyl)-1,1-dioxo-1,2,5-thiadiazolidin-3-one (2, 143.09 mg, 277.62 μmol) was added and stirred for 16 h. The solvent was removed under reduced pressure and the residue was treated with water (5 mL). The precipitate was filtered and dried to obtain N-[7-benzyloxy-5-fluoro-6-(1,1,4-trioxo-1,2,5-thiadiazolidin-2-yl)-2-naphthyl]-2-[1-[3-(2,6-dioxo-3-piperidyl)-1-methyl-indazol-6-yl]-4-piperidyl]acetamide (3, 300 mg, 239.51 μmol, 54% yield, 61% purity) as a colorless solid. LCMS (ES+): m/z 769.2 [M+H]⁺

### Step 2: 2-[1-[3-(2,6-dioxo-3-piperidyl)-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 167)

To a stirred solution of N-[7-benzyloxy-5-fluoro-6-(1,1,4-trioxo-1,2,5-thiadiazolidin-2-yl)-2-naphthyl]-2-[1-[3-(2,6-dioxo-3-piperidyl)-1-methyl-indazol-6-yl]-4-piperidyl]acetamide (3, 300 mg, 239.51 μmol) in DCM (5 mL) and toluene (5 mL) was added pentamethylbenzene (177.54 mg, 1.20 mmol, 193.60 μL) at −78° C. Then boron trichloride (1.0 M in DCM) (561.26 mg, 4.79 mmol) was added. The resulting solution was stirred at RT for 4 h. The reaction was quenched with 5% MeOH in DCM (10 mL) and solvent removed. The residue was triturated with MTBE (50 mL) and purified by reverse phase prep HPLC [Purification method: Column: XBridge C-18 (20×150 mm); Mobile phase: A: 0.1% formic acid in water; Mobile phase B: MeCN] to afford 2-[1-[3-(2,6-dioxo-3-piperidyl)-1-methyl-indazol-6-yl]-4-piperidyl]-N-[5-fluoro-7-hydroxy-6-(1,1,4-trioxo-1,2,5-thiadiazolidin-2-yl)-2-naphthyl]acetamide (Example 167, 36 mg, 48.77 μmol, 20% yield, formic acid salt) as a colorless solid. LCMS (ES+): m/z 678.0 [M+H]⁺

¹H NMR (400 MHZ, DMSO-d₆) δ 10.86 (s, 1H), 10.21-10.11 (m, 2H), 8.18 (s, 1H), 7.84 (d, J=8.9 Hz, 1H), 7.53 (d, J=8.8 Hz, 1H), 7.43 (d, J=9.0 Hz, 1H), 7.02-6.91 (m, 3H), 4.29-4.20 (m, 3H), 3.90 (s, 3H), 3.78 (d, J=12.2 Hz, 2H), 2.89 (t, J=12.1 Hz, 2H), 2.69-2.57 (m, 3H), 2.40-2.25 (m, 3H), 2.20-2.10 (m, 1H), 2.10-1.98 (m, 1H), 1.85 (d, J=12.7 Hz, 2H), 1.54-1.38 (m, 2H).

### N-(6-(1,1-dioxido-4-oxo-1,2,5-thiadiazolidin-2-yl)-5-fluoro-7-hydroxynaphthalen-2-yl)-2-(6-((1-(2,6-dioxopiperidin-3-yl)-3-methyl-2-oxo-2,3-dihydro-1H-benzo[d]imidazol-5-yl)amino)pyridin-3-yl)acetamide (Example 168)

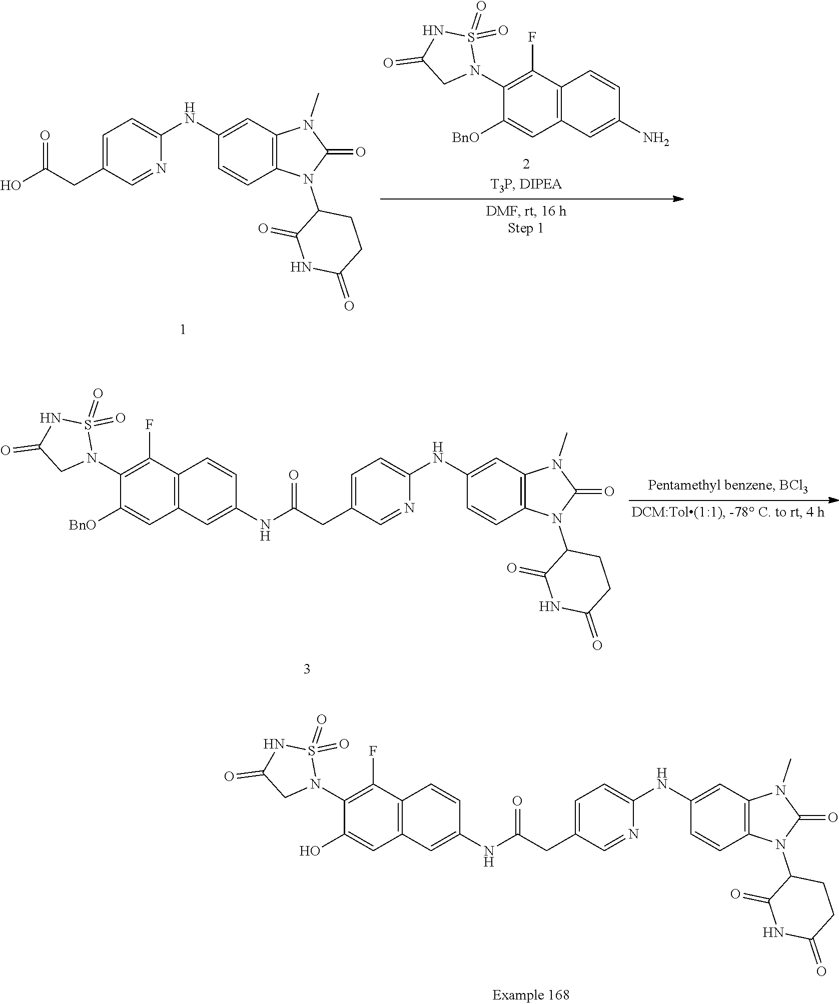 [SMILES: Cn1c(=O)n(C2CCC(=O)NC2=O)c2ccc(Nc3ccc(CC(=O)O)cn3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(Nc3ccc(CC(=O)Nc4ccc5c(F)c(N6CC(=O)NS6(=O)=O)c(OCc6ccccc6)cc5c4)cn3)cc21 ; Cn1c(=O)n(C2CCC(=O)NC2=O)c2ccc(Nc3ccc(CC(=O)Nc4ccc5c(F)c(N6CC(=O)NS6(=O)=O)c(O)cc5c4)cn3)cc21]

### Step 1: N-(7-(benzyloxy)-6-(1,1-dioxido-4-oxo-1,2,5-thiadiazolidin-2-yl)-5-fluoronaphthalen-2-yl)-2-(6-((1-(2,6-dioxopiperidin-3-yl)-3-methyl-2-oxo-2,3-dihydro-1H-benzo[d]imidazol-5-yl)amino)pyridin-3-yl)acetamide (3)

To a solution of 2-(6-((1-(2,6-dioxopiperidin-3-yl)-3-methyl-2-oxo-2,3-dihydro-1H-benzo[d]imidazol-5-yl)amino)pyridin-3-yl)acetic acid (1, 250 mg, 610.66 μmol, TFA salt) and 5-(6-amino-3-(benzyloxy)-1-fluoronaphthalen-2-yl)-1,2,5-thiadiazolidin-3-one 1,1-dioxide (2, 245.12 mg, 610.66 μmol) in DMF (5 mL) were added DIPEA (1.18 g, 9.16 mmol, 1.60 mL) and T₃P (50% in ethyl acetate) (2.72 g, 4.27 mmol). The reaction was stirred at 50° C. for 16 h. The reaction mixture was filtered and concentrated under reduced pressure. The residue was purified by prep-HPLC (flow: 60 mL/min; gradient: from 15-45% MeCN-water (0.1% TFA) over 10 min; column: Phenomenex luna C18 150×40 mm×15 μm) to afford N-(7-(benzyloxy)-6-(1,1-dioxido-4-oxo-1,2,5-thiadiazolidin-2-yl)-5-fluoronaphthalen-2-yl)-2-(6-((1-(2,6-dioxopiperidin-3-yl)-3-methyl-2-oxo-2,3-dihydro-1H-benzo[d]imidazol-5-yl)amino)pyridin-3-yl)acetamide (3, 280 mg, 305.69 μmol, 50% yield, TFA salt) as a yellow solid. LCMS (ESI): m/z 793.3 [M+H]⁺

### Step 2: N-(6-(1,1-dioxido-4-oxo-1,2,5-thiadiazolidin-2-yl)-5-fluoro-7-hydroxynaphthalen-2-yl)-2-(6-((1-(2,6-dioxopiperidin-3-yl)-3-methyl-2-oxo-2,3-dihydro-1H-benzo[d]imidazol-5-yl)amino)pyridin-3-yl)acetamide (Example 168)

To a solution of N-(7-(benzyloxy)-6-(1,1-dioxido-4-oxo-1,2,5-thiadiazolidin-2-yl)-5-fluoronaphthalen-2-yl)-2-(6-((1-(2,6-dioxopiperidin-3-yl)-3-methyl-2-oxo-2,3-dihydro-1H-benzo[d]imidazol-5-yl)amino)pyridin-3-yl)acetamide (3, 130 mg, 163.98 μmol, TFA salt) in toluene (1 mL) and DCM (1 mL) was added 1,2,3,4,5-pentamethylbenzene (72.93 mg, 491.93 μmol, 79.53 μL). Then BCl₃ (1 M in DCM, 4.92 mL) was added to the mixture at −78° C. The mixture was stirred at RT for 4 h. A solution of DCM:MeOH=10:1(10 mL) was added at −78° C. The reaction mixture was filtered and concentrated under reduced pressure and the residue was purified by prep-HPLC (flow: 25 mL/min; gradient: from 12-42% MeCN-water (0.1% TFA) over 7 min; column: 3_Phenomenex Luna C18 75×30 mm×3 μm), to afford N-(6-(1,1-dioxido-4-oxo-1,2,5-thiadiazolidin-2-yl)-5-fluoro-7-hydroxynaphthalen-2-yl)-2-(6-((1-(2,6-dioxopiperidin-3-yl)-3-methyl-2-oxo-2,3-dihydro-1H-benzo[d]imidazol-5-yl)amino)pyridin-3-yl)acetamide (Example 168, 68.2 mg, 82.67 μmol, 50% yield, TFA salt) as a yellow solid. LCMS (ESI): m/z 703.3 [M+H]⁺

¹H NMR (400 MHZ, DMSO-d₆) δ 11.12 (s, 1H), 10.43 (s, 1H), 10.14 (s, 1H), 8.17-8.07 (m, 1H), 7.92 (s, 1H), 7.89-7.84 (m, 2H), 7.47-7.42 (m, 2H), 7.19 (d, J=8.4 Hz, 1H), 7.10 (dd, J=8.4, 2.0 Hz, 1H), 7.06 (d, J=9.1 Hz, 1H), 6.95 (s, 1H), 5.40 (dd, J=12.8, 5.4 Hz, 1H), 4.27 (s, 2H), 3.71 (s, 2H), 3.35 (s, 3H), 2.98-2.85 (m, 1H), 2.81-2.59 (m, 2H), 2.09-1.98 (m, 1H).

### N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-3-methylphenyl)acetamide (Example 169)

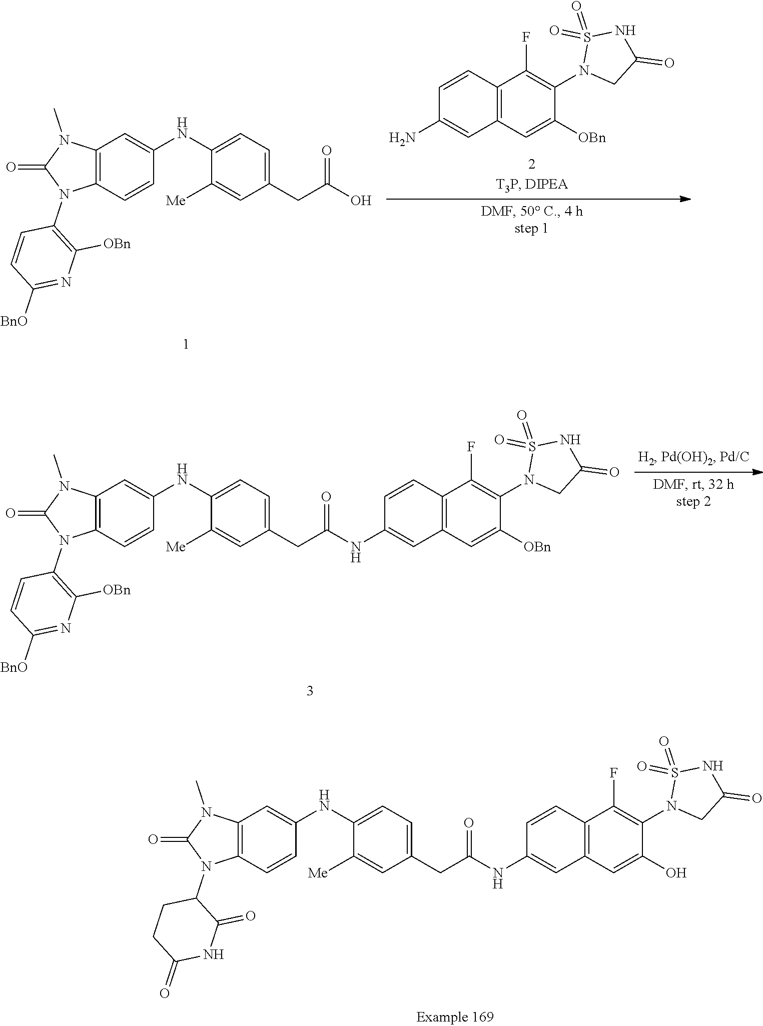 [SMILES: Cc1cc(CC(=O)O)ccc1Nc1ccc2c(c1)n(C)c(=O)n2-c1ccc(OCc2ccccc2)nc1OCc1ccccc1 ; Nc1ccc2c(F)c(N3CC(=O)NS3(=O)=O)c(OCc3ccccc3)cc2c1 ; Cc1cc(CC(=O)Nc2ccc3c(F)c(N4CC(=O)NS4(=O)=O)c(OCc4ccccc4)cc3c2)ccc1Nc1ccc2c(c1)n(C)c(=O)n2-c1ccc(OCc2ccccc2)nc1OCc1ccccc1 ; Cc1cc(CC(=O)Nc2ccc3c(F)c(N4CC(=O)NS4(=O)=O)c(O)cc3c2)ccc1Nc1ccc2c(c1)n(C)c(=O)n2C1CCC(=O)NC1=O]

### Step 1: N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-3-methylphenyl)acetamide (3)

To a mixture of 2-(4-((1-(2,6-bis(benzyloxy)pyridin-3-yl)-3-methyl-2-oxo-2,3-dihydro-1H-benzo[d]imidazol-5-yl)amino)-3-methylphenyl)acetic acid (1, 310 mg, 516.10 μmol) and 5-(6-amino-3-(benzyloxy)-1-fluoronaphthalen-2-yl)-1,2,5-thiadiazolidin-3-one 1,1-dioxide (2, 207.17 mg, 516.10 μmol) in DMF (6 mL) were added DIPEA (2.00 g, 15.48 mmol, 2.70 mL) and T₃P (50% in ethyl acetate) (3.28 g, 5.16 mmol). The mixture was stirred at 50° C. for 4 h. The reaction mixture was diluted with water (30 mL) and extracted with ethyl acetate (20 mL×2). The combined organic layers were washed with brine (30 mL×2), dried over Na₂SO₄, filtered and concentrated under reduced pressure. The residue was purified by column chromatography (SiO₂, DCM/MeOH=50/1 to 5/1) and prep-TLC (SiO₂, ethyl acetate:EtOH=5:1, R_{f}=0.3) to afford N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-3-methylphenyl)acetamide (3, 400 mg, 390.22 μmol, 76% yield) as a brown solid. LCMS (ESI): m/z 983.9 [M+H]⁺

### Step 2: N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-3-methylphenyl)acetamide (Example 169)

To a solution of N-(7-(benzyloxy)-6-(1,1-dioxido-4-oxo-1,2,5-thiadiazolidin-2-yl)-5-fluoronaphthalen-2-yl)-2-(4-((1-(2,6-bis(benzyloxy)pyridin-3-yl)-3-methyl-2-oxo-2,3-dihydro-1H-benzo[d]imidazol-5-yl)amino)-3-methylphenyl)acetamide (3, 350 mg, 355.67 μmol) in DMF (3 mL) were added Pd/C (30 mg, 10%) and Pd(OH)₂ (30 mg, 10%). The mixture was stirred at 25° C. for 32 h under H₂ (15 psi). The mixture was filtered and washed with DMF (1 mL×2). The filtrate was purified by prep-HPLC (flow: 25 mL/min; gradient: from 9-41% MeCN in water (10 mM NH₄HCO₃) over 10 min; column: Waters Xbridge 150×25 mm×5 μm) and (flow: 25 mL/min; gradient: from 31-61% MeCN in water (0.1% TFA) over 10 min; column: 3_Phenomenex Luna C18 75×30 mm×3 μm) to afford N-(6-(1,1-dioxido-4-oxo-1,2,5-thiadiazolidin-2-yl)-5-fluoro-7-hydroxynaphthalen-2-yl)-2-(4-((1-(2,6-dioxopiperidin-3-yl)-3-methyl-2-oxo-2,3-dihydro-1H-benzo[d]imidazol-5-yl)amino)-3-methylphenyl)acetamide (Example 169, 71 mg, 84.71 μmol, 24% yield, TFA salt) as a white solid. LCMS (ESI): m/z 716.0 [M+H]⁺

¹H NMR (400 MHZ, DMSO-d₆) δ 11.07 (s, 1H), 10.49 (s, 1H), 10.34 (s, 1H), 8.18 (s, 1H), 7.86 (d, J=9.0 Hz, 1H), 7.46 (dd, J=9.1, 1.9 Hz, 1H), 7.14 (s, 1H), 7.05 (d, J=1.3 Hz, 2H), 6.97-6.92 (m, 2H), 6.77 (d, J=2.1 Hz, 1H), 6.65 (dd, J=8.4, 2.1 Hz, 1H), 5.30 (dd, J=12.8, 5.3 Hz, 1H), 4.44 (s, 2H), 3.26 (s, 3H), 2.98-2.83 (m, 1H), 2.75-2.57 (m, 2H), 2.21 (s, 3H), 2.07-1.96 (m, 1H).

### N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)piperidin-1-yl)propanamide (Example 170)

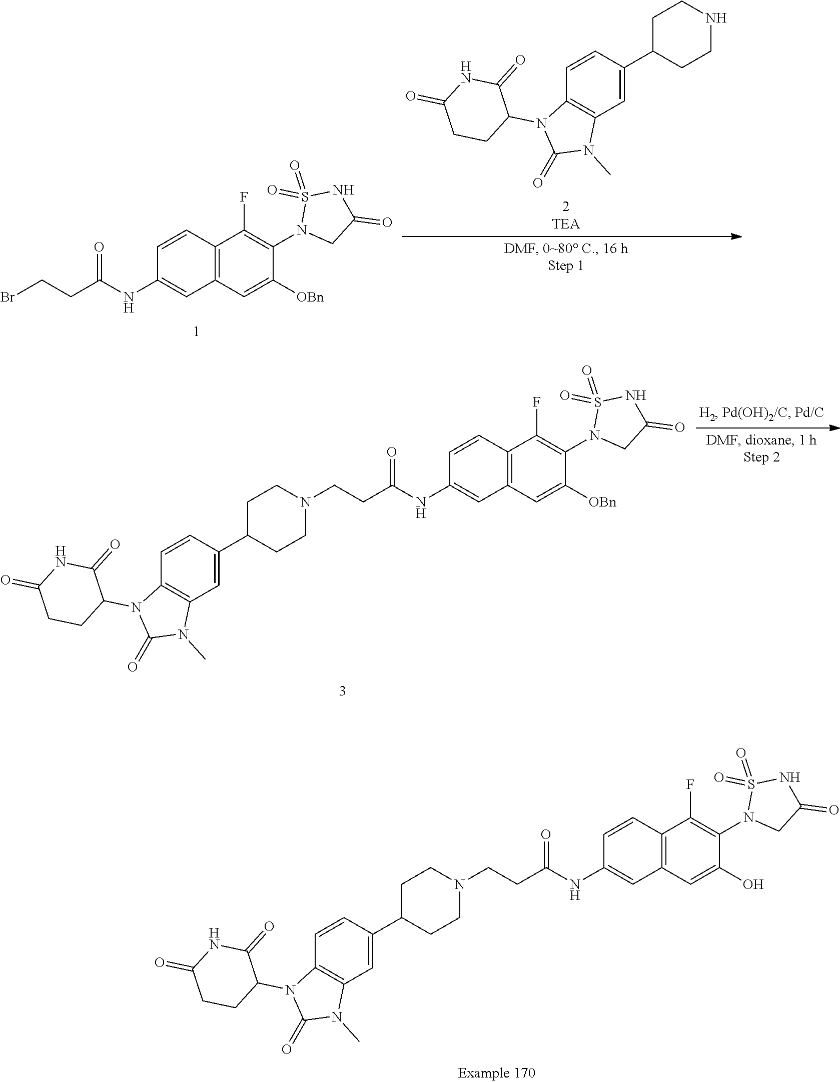 [SMILES: O=C(CCBr)Nc1ccc2c(F)c(N3CC(=O)NS3(=O)=O)c(OCc3ccccc3)cc2c1 ; Cn1c(=O)n(C2CCC(=O)NC2=O)c2ccc(C3CCNCC3)cc21 ; Cn1c(=O)n(C2CCC(=O)NC2=O)c2ccc(C3CCN(CCC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN(CCC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)piperidin-1-yl)propanamide (3)

To a mixture of 3-(3-methyl-2-oxo-5-(piperidin-4-yl)-2,3-dihydro-1H-benzo[d]imidazol-1-yl)piperidine-2,6-dione (2, 98.31 mg, 287.12 μmol) and TEA (66.03 mg, 652.53 μmol, 90.95 μL) in DMF (3 mL) was added N-(7-(benzyloxy)-6-(1,1-dioxido-4-oxo-1,2,5-thiadiazolidin-2-yl)-5-fluoronaphthalen-2-yl)-3-bromopropanamide (1, 140 mg, 261.01 μmol) at 0° C.) The mixture was stirred at 80° C. for 16 h. The reaction mixture was purified by prep-HPLC (flow: 25 mL/min; gradient: from 17-47% MeCN in water (0.1% TFA): column: Phenomenex Synergi C18 150×25 mm×10 μm) to afford N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)piperidin-1-yl)propanamide (3, 140 mg, 153.53 μmol, 59% yield, TFA salt) as white solid. LCMS (ESI): m/z 798.4 [M+H]⁺

### Step 2: N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)piperidin-1-yl)propanamide (Example 170)

To a solution of N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)piperidin-1-yl)propanamide (3, 130 mg, 162.94 μmol) in 1,4-dioxane (2 mL) and DMF (2 mL) was added Pd/C (6 mg, 10% purity) and Pd(OH)₂/C (6 mg, 10% purity) under H₂, then the mixture was stirred at 20° C. for 1 h under H₂ (15 psi) atmosphere. The mixture was filtered and concentrated under reduced pressure. The residue was purified by prep-HPLC (flow: 25 mL/min; gradient: from 12-42% MeCN in water (0.1% TFA); column: Phenomenex Synergi C18 150×25 mm×10 μm) to afford N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)piperidin-1-yl)propanamide (Example 170, 58.14 mg, 70.75 μmol, 43% yield, TFA salt) as off-white solid. LCMS (ESI): m/z 708.2 [M+H]⁺

¹H NMR (400 MHZ, d₆-DMSO) δ 11.11 (s, 1H), 10.46 (s, 1H), 9.88 (br s, 1H), 9.31-9.19 (m, 1H), 8.16 (s, 1H), 7.86 (d, J=8.8 Hz, 1H), 7.45 (br d, J=9.2 Hz, 1H), 7.09-7.04 (m, 2H), 6.97-6.90 (m, 2H), 5.36 (br dd, J=5.4, 12.8 Hz, 1H), 4.11 (s, 2H), 3.65 (br d, J=10.2 Hz, 2H), 3.46 (br d, J=4.4 Hz, 2H), 3.34 (s, 3H), 3.14 (br d, J=10.2 Hz, 2H), 2.98-2.94 (m, 2H), 2.88 (br s, 1H), 2.66 (br d, J=13.2 Hz, 2H), 2.08-1.90 (m, 6H).

### 3-(5-(1-(2-(3-((7-(1,1-dioxido-4-oxo-1,2,5-thiadiazolidin-2-yl)-8-fluoro-6-hydroxynaphthalen-2-yl)amino)azetidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Example 171)

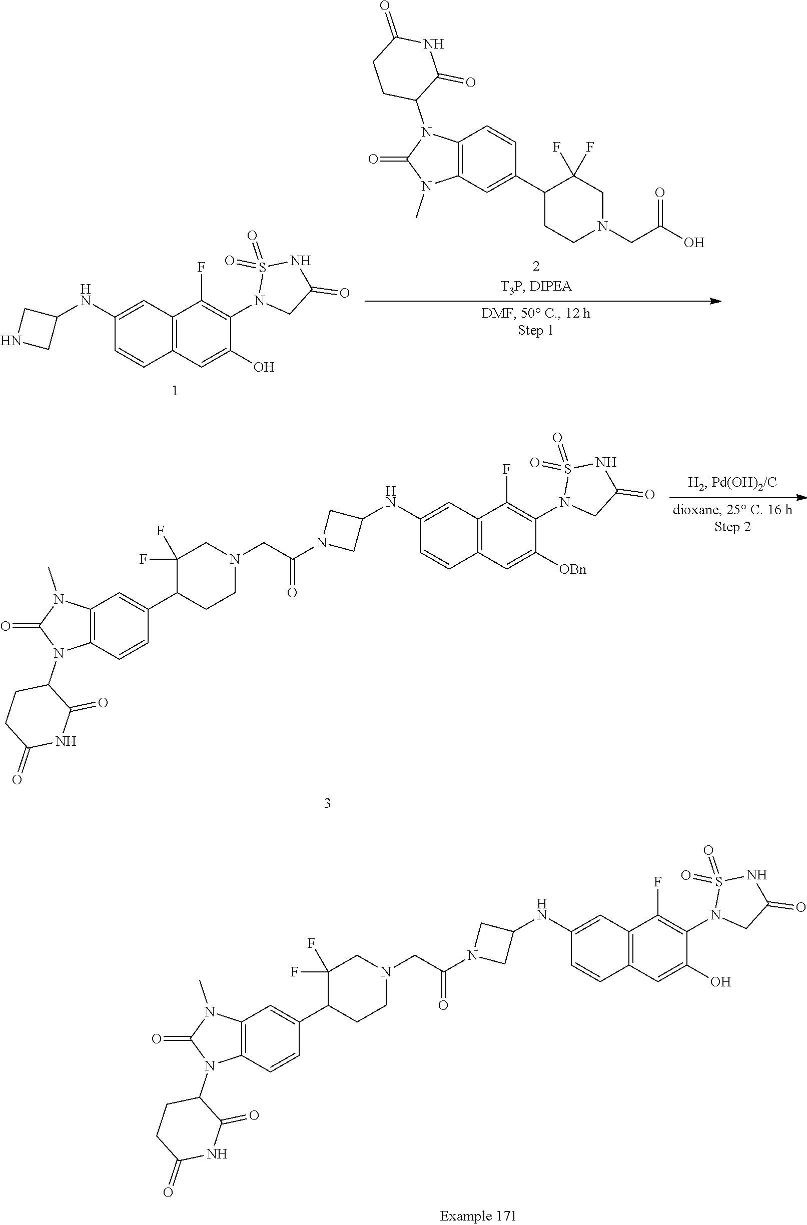 [SMILES: O=C1CN(c2c(O)cc3ccc(NC4CNC4)cc3c2F)S(=O)(=O)N1 ; Cn1c(=O)n(C2CCC(=O)NC2=O)c2ccc(C3CCN(CC(=O)O)CC3(F)F)cc21 ; Cn1c(=O)n(C2CCC(=O)NC2=O)c2ccc(C3CCN(CC(=O)N4CC(Nc5ccc6cc(OCc7ccccc7)c(N7CC(=O)NS7(=O)=O)c(F)c6c5)C4)CC3(F)F)cc21 ; Cn1c(=O)n(C2CCC(=O)NC2=O)c2ccc(C3CCN(CC(=O)N4CC(Nc5ccc6cc(O)c(N7CC(=O)NS7(=O)=O)c(F)c6c5)C4)CC3(F)F)cc21]

### Step 1: 3-(5-(1-(2-(3-((6-(benzyloxy)-7-(1,1-dioxido-4-oxo-1,2,5-thiadiazolidin-2-yl)-8-fluoronaphthalen-2-yl)amino)azetidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (3)

To a solution of 2-(4-(1-(2,6-dioxopiperidin-3-yl)-3-methyl-2-oxo-2,3-dihydro-1H-benzo[d]imidazol-5-yl)-3,3-difluoropiperidin-1-yl)acetic acid (2, 150 mg, 317.21 μmol, HCl salt) in DMF (10 mL) was added 2,4,6-tripropyl-1,3,5,2,4,6-trioxatriphosphinane 2,4,6-trioxide (1.41 g, 2.22 mmol, 50% purity), N-ethyl-N-isopropylpropan-2-amine (615 mg, 4.76 mmol, 828.84 μL) and 5-(7-(azetidin-3-ylamino)-1-fluoro-3-hydroxynaphthalen-2-yl)-1,2,5-thiadiazolidin-3-one 1,1-dioxide (1, 213 mg, 317.35 μmol, TFA salt). The mixture was stirred at 50° C. for 12 h. The mixture was concentrate under reduced pressure. The residue was purified by prep-HPLC (flow: 60 mL/min; gradient: from 10-55% MeCN in water (0.1% FA) to afford 3-(5-(1-(2-(3-((6-(benzyloxy)-7-(1,1-dioxido-4-oxo-1,2,5-thiadiazolidin-2-yl)-8-fluoronaphthalen-2-yl)amino)azetidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (3, 70 mg, 72.97 μmol, 23% yield, Formic acid salt) as a yellow solid. LCMS (ESI): m/z 875.2 [M+H]⁺

### Step 2: 3-(5-(1-(2-(3-((7-(1,1-dioxido-4-oxo-1,2,5-thiadiazolidin-2-yl)-8-fluoro-6-hydroxynaphthalen-2-yl)amino)azetidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Example 171)

To a solution of 3-(5-(1-(2-(3-((6-(benzyloxy)-7-(1,1-dioxido-4-oxo-1,2,5-thiadiazolidin-2-yl)-8-fluoronaphthalen-2-yl)amino)azetidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (3, 70 mg, 80.01 μmol) in dioxane (10 mL) was added Pd(OH)₂/C (35 mg, 56.71 μmol). The mixture was stirred for 16 h under H₂ (15 psi) atmosphere. The mixture was filtered and washed with DMF (1 mL). The filtrate was purified by prep-HPLC (flow: 25 mL/min; gradient: from 18-38% water (0.1% TFA) in MeCN over 7 min; column: Phenomenex Luna C18 75×30 mm×3 μm) to afford 3-(5-(1-(2-(3-((7-(1,1-dioxido-4-oxo-1,2,5-thiadiazolidin-2-yl)-8-fluoro-6-hydroxynaphthalen-2-yl)amino)azetidin-1-yl)-2-oxoethyl)-3,3-difluoropiperidin-4-yl)-3-methyl-2-oxo-2,3-dihydro-1H-benzo[d]imidazol-1-yl)piperidine-2,6-dione (Example 171, 14.39 mg, 15.69 μmol, 20% yield, TFA salt) as a gray solid. LCMS (ESI): m/z 785.4 [M+H]⁺

¹H NMR (400 MHZ, DMSO-d₆) δ 11.10 (s, 1H), 9.65 (s, 1H), 7.56 (d, J=9.0 Hz, 1H), 7.13-7.05 (m, 2H), 7.04-6.96 (m, 2H), 6.94 (s, 1H), 6.53 (s, 1H), 5.36 (dd, J=12.8, 5.4 Hz, 1H), 4.68-4.52 (m, 1H), 4.39-4.33 (m, 2H), 4.30 (s, 2H), 4.03-3.93 (m, 1H), 3.86-3.77 (m, 1H), 3.34 (s, 3H), 3.28-3.19 (m, 2H), 2.96-2.85 (m, 1H), 2.82-2.58 (m, 2H), 2.36-2.24 (m, 1H), 2.07-1.98 (m, 1H), 1.98-1.87 (m, 1H). Note: additional peaks under solvent signal.

### 2-[4-[1-(2,6-dioxo-3-piperidyl)-3-methyl-2-oxo-benzimidazol-5-yl]pyrazol-1-yl]acetic acid (5)

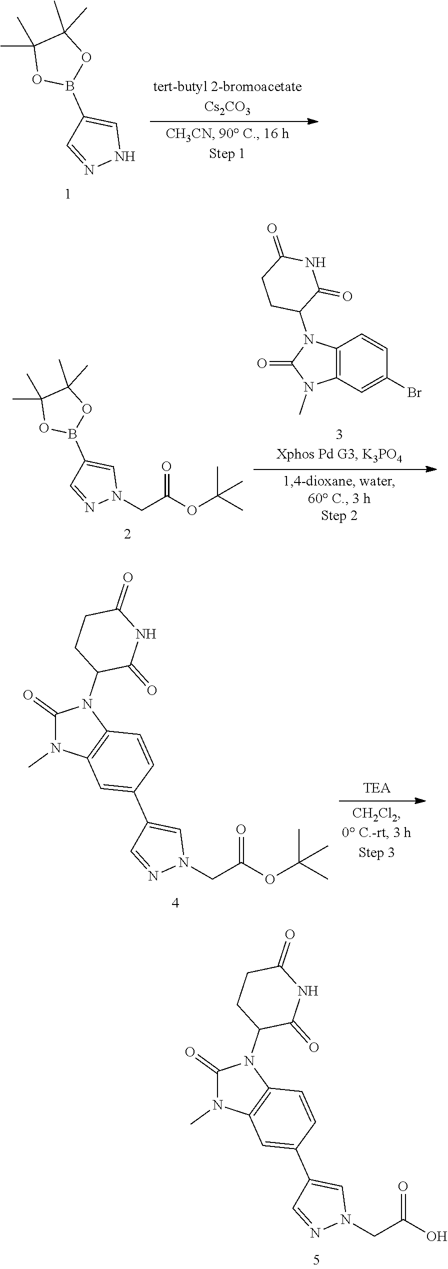

### Step 1: tert-butyl 2-[4-(4,4,5,5-tetramethyl-1,3,2-dioxaborolan-2-yl)pyrazol-1-yl]acetate (2)

In to a 25 mL single neck round bottom flask containing a well-stirred solution of 4-(4,4,5,5-tetramethyl-1,3,2-dioxaborolan-2-yl)-1H-pyrazole (1, 4 g, 20.61 mmol) in anhydrous CH₃CN (40 mL) was added Cs₂CO₃ (10.07 g, 30.92 mmol) followed by tert-butyl 2-bromoacetate (4.02 g, 20.61 mmol, 3.02 mL). The mixture was stirred for 16 h at 90° C. The reaction mixture was diluted with water (50 mL) and extracted with EtOAc (2×100 mL). The combined organic layers were dried over Na₂SO₄, filtered and concentrated to afford tert-butyl 2-[4-(4,4,5,5-tetramethyl-1,3,2-dioxaborolan-2-yl)pyrazol-1-yl]acetate (2, 4 g, 8.94 mmol, 43% yield) as a yellow oil. The material was used in the next step without further purification. LCMS (ES+): m/z 309.2 [M+H]⁺

### Step 2: tert-butyl 2-[4-[1-(2,6-dioxo-3-piperidyl)-3-methyl-2-oxo-benzimidazol-5-yl]pyrazol-1-yl]acetate (4)

Into a 100 mL sealed tube containing a well-stirred solution of 3-(5-bromo-3-methyl-2-oxo-benzimidazol-1-yl)piperidine-2,6-dione (3, 400 mg, 1.18 mmol) and tert-butyl 2-[4-(4,4,5,5-tetramethyl-1,3,2-dioxaborolan-2-yl)pyrazol-1-yl]acetate (2, 528.32 mg, 1.18 mmol, 000) in 1,4-dioxane (8 mL) and water (0.8 mL) was added K₃PO₄ (753.27 mg, 3.55 mmol) and the resulting contents were degassed by with nitrogen gas for 10 min. XPhos Pd G3 (150.19 mg, 177.43 μmol) was added to the reaction mixture and heated at 60° C. for 3 h. The reaction mixture was concentrated to dryness and purified by flash silica-gel (230-400 mesh) column chromatography (85% EtOAc in pet. ether) to afford tert-butyl 2-[4-[1-(2,6-dioxo-3-piperidyl)-3-methyl-2-oxo-benzimidazol-5-yl]pyrazol-1-yl]acetate (4, 400 mg, 800.98 μmol, 68% yield) as an off-white solid. LCMS (ES+): m/z 440.2 [M+H]⁺

### Step 3: 2-[4-[1-(2,6-dioxo-3-piperidyl)-3-methyl-2-oxo-benzimidazol-5-yl]pyrazol-1-yl]acetic acid (5)

In to a 50 mL single neck round bottom flask containing a well-stirred solution of tert-butyl 2-[4-[1-(2,6-dioxo-3-piperidyl)-3-methyl-2-oxo-benzimidazol-5-yl]pyrazol-1-yl]acetate (4, 600 mg, 1.20 mmol) in anhydrous DCM (3 mL) was added dropwise TFA (3.91 g, 34.27 mmol, 2.64 mL) at 0° C. under nitrogen atmosphere. After 3 h at RT, the reaction mixture was concentrated and triturated with Et₂O (2×10 mL) to afford 2-[4-[1-(2,6-dioxo-3-piperidyl)-3-methyl-2-oxo-benzimidazol-5-yl]pyrazol-1-yl]acetic acid (5, 700 mg, 1.17 mmol, 97% yield, TFA salt) as a off white solid. LCMS (ES+): m/z 384.2 [M+H]⁺

### 3-[4-[1-(2,6-dioxo-3-piperidyl)-3-methyl-2-oxo-benzimidazol-5-yl] pyrazol-1-yl] propionic acid (6)

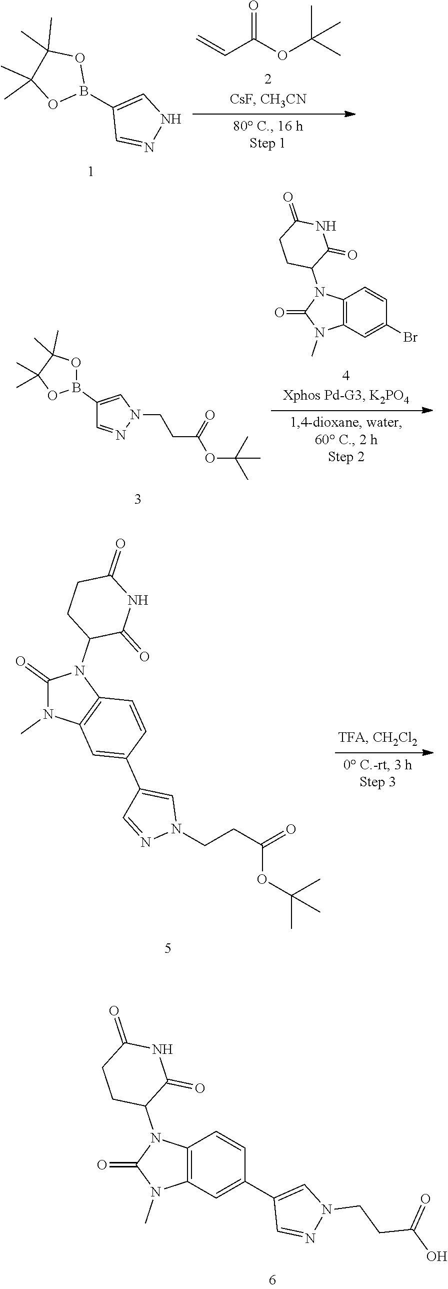

### Step 1: tert-butyl 3-[4-(4,4,5,5-tetramethyl-1,3,2-dioxaborolan-2-yl) pyrazol-1-yl]propanoate (3)

Into a 100 mL sealed tube containing a well-stirred solution of 4-(4,4,5,5-tetramethyl-1,3,2-dioxaborolan-2-yl)-1H-pyrazole (1, 2 g, 10.31 mmol) in anhydrous CH₃CN (25 mL) was added CsF (2.35 g, 15.46 mmol). After 10 minutes, tert-butyl prop-2-enoate (2, 1.59 g, 12.37 mmol, 1.80 mL) was added and the reaction mixture was heated at 80° C. for 16 h. Afterwards, the reaction mixture was diluted with water (200 mL), extracted with ethyl acetate (2×75 mL). The combined organic layers were washed with brine solution (50 mL), dried over Na₂SO₄ and concentrated under reduced pressure to obtain crude material that was triturated with MTBE (25 mL) to afford tert-butyl 3-[4-(4,4,5,5-tetramethyl-1,3,2-dioxaborolan-2-yl) pyrazol-1-yl]propanoate (3, 2.5 g, 6.99 mmol, 68% yield) as a thick syrup. LCMS (ES+): m/z 323 [M+H]⁺

### Step 2: tert-butyl 3-[4-[1-(2,6-dioxo-3-piperidyl)-3-methyl-2-oxo-benzimidazol-5-yl]pyrazol-1-yl]propanoate (5)

Into a 50 mL sealed tube containing a well-stirred solution of tert-butyl 3-[4-(4,4,5,5-tetramethyl-1,3,2-dioxaborolan-2-yl) pyrazol-1-yl]propanoate (3, 600 mg, 1.68 mmol) in 1,4-dioxane (10 mL) and water (0.9 mL) were added 3-(5-bromo-3-methyl-2-oxo-benzimidazol-1-yl)piperidine-2,6-dione (4, 566.73 mg, 1.68 mmol) and anhydrous potassium phosphate (1.07 g, 5.03 mmol). The reaction mixture was purged with nitrogen gas for 10 minutes. XPhos Pd G3 (212.68 mg, 251.39 μmol) was added and the reaction mixture was heated at 60° C. for 3 h. The reaction mixture was filtered through Celite and washed with EtOAc (250 mL). The filtrate was concentrated under reduced pressure and purified by silica gel column chromatography (100-200 mesh, 50 g; 0-10% MeOH in DCM) to afford tert-butyl 3-[4-[1-(2,6-dioxo-3-piperidyl)-3-methyl-2-oxo-benzimidazol-5-yl]pyrazol-1-yl]propanoate (5, 460 mg, 869.81 μmol, 52% yield) as pale yellow solid. LCMS (ES+): m/z 454.2 [M+H]⁺

### Step 3: 3-[4-[1-(2,6-dioxo-3-piperidyl)-3-methyl-2-oxo-benzimidazol-5-yl] pyrazol-1-yl] propionic acid (6)

Into a 25 mL single neck round bottom flask containing a well-stirred solution of tert-butyl 3-[4-[1-(2,6-dioxo-3-piperidyl)-3-methyl-2-oxo-benzimidazol-5-yl]pyrazol-1-yl]propanoate (5, 400 mg, 756.36 μmol) in dry DCM (3 mL) at 0° C. under nitrogen atmosphere was added TFA (1.72 g, 15.13 mmol, 1.17 mL) dropwise. After 2 h at RT, the reaction mixture was concentrated under reduced pressure and purified by preparative HPLC (Column: X-select C18 (150×19) mm 5 μm; Mobile Phase A: 0.1% TFA in Water and Mobile Phase B: CH₃CN) to afford 3-[4-[1-(2,6-dioxo-3-piperidyl)-3-methyl-2-oxo-benzimidazol-5-yl] pyrazol-1-yl] propionic acid (6, 360 mg, 675.92 μmol, 89% yield, TFA salt) as a white solid. LCMS (ES+): m/z 398.2 [M+H]⁺

### 2-(4-(1-(2,6-dioxopiperidin-3-yl)-3-ethyl-2-oxo-2,3-dihydro-1H-benzo[d]imidazol-5-yl)piperidin-1-yl)acetic acid (9)

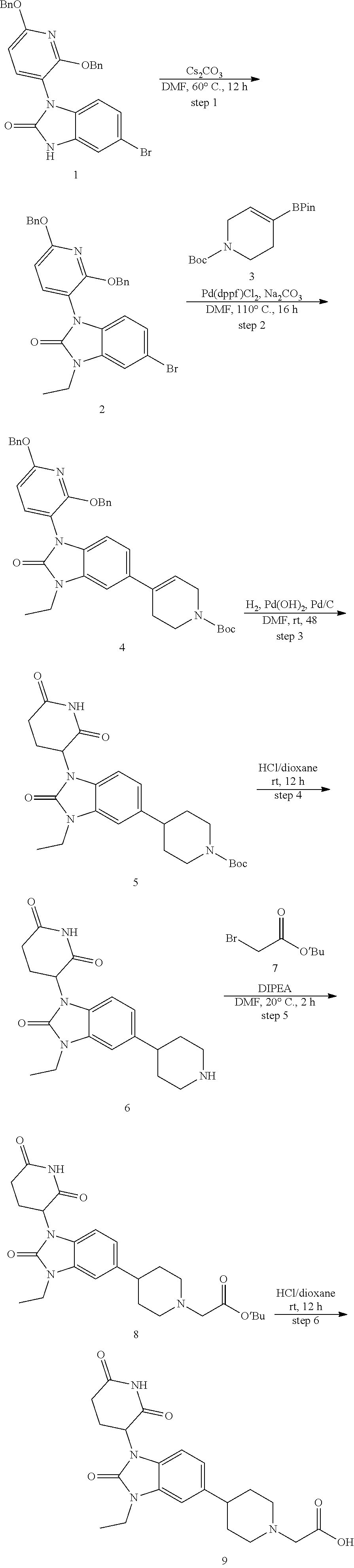

### Step 1: 1-(2,6-bis(benzyloxy)pyridin-3-yl)-5-bromo-3-ethyl-1H-benzo[d]imidazol-2(3H)-one (2)

To a solution of 6-bromo-3-(2,6-dibenzyloxy-3-pyridyl)-1H-benzimidazol-2-one (1, 1 g, 1.99 mmol) in DMF (10 mL) were added iodoethane (931.40 mg, 5.97 mmol, 480.10 μL) and Cs₂CO₃ (1.62 g, 4.98 mmol). The mixture was stirred at 60° C. for 12 h. The reaction mixture was quenched with H₂O (10 mL) and extracted with ethyl acetate (30 mL). The organic layer was washed with brine (10 mL×5), dried over Na₂SO₄, filtered and concentrated under reduced pressure. The residue was purified by column chromatography (SiO₂, petroleum ether/ethyl acetate=20/1 to 1/1) to afford 1-(2,6-bis(benzyloxy)pyridin-3-yl)-5-bromo-3-ethyl-1H-benzo[d]imidazol-2(3H)-one (2, 0.9 g, 1.70 mmol, 85% yield) as a white solid.

### Step 2: tert-butyl 4-(1-(2,6-bis(benzyloxy)pyridin-3-yl)-3-ethyl-2-oxo-2,3-dihydro-1H-benzo[d]imidazol-5-yl)-5,6-dihydropyridine-1(2H)-carboxylate (4)

A mixture of 1-(2,6-bis(benzyloxy)pyridin-3-yl)-5-bromo-3-ethyl-1H-benzo[d]imidazol-2(3H)-one (2, 0.7 g, 1.32 mmol), tert-butyl 4-(4,4,5,5-tetramethyl-1,3,2-dioxaborolan-2-yl)-3,6-dihydro-2H-pyridine-1-carboxylate (3, 530.49 mg, 1.72 mmol), Pd(dppf)Cl₂ (96.57 mg, 131.97 μmol), and Na₂CO₃ (419.63 mg, 3.96 mmol, 165.86 μL) in water (2 mL) and dioxane (20 mL) was degassed and purged with N₂ 3 times. The mixture was stirred at 110° C. for 16 h under N₂ atmosphere. The reaction mixture was concentrated under reduced pressure and the residue was dissolved in DCM (20 mL) and the dried over Na₂SO₄ and filtered. The solvent was removed and the residue was purified by column chromatography (SiO₂, Petroleum ether: Ethyl acetate=20:1 to 1:1) to afford 4-(1-(2,6-bis(benzyloxy)pyridin-3-yl)-3-ethyl-2-oxo-2,3-dihydro-1H-tert-butyl benzo[d]imidazol-5-yl)-5,6-dihydropyridine-1(2H)-carboxylate (4, 390 mg, 617.73 μmol, 47% yield) as a white solid. LCMS (ESI): m/z 633.4 [M+H]⁺

### Step 3: tert-butyl 4-(1-(2,6-dioxopiperidin-3-yl)-3-ethyl-2-oxo-2,3-dihydro-1H-benzo[d]imidazol-5-yl)piperidine-1-carboxylate (5)

To a solution of tert-butyl 4-(1-(2,6-bis(benzyloxy)pyridin-3-yl)-3-ethyl-2-oxo-2,3-dihydro-1H-benzo[d]imidazol-5-yl)-5,6-dihydropyridine-1(2H)-carboxylate (4, 1 g, 1.58 mmol) in DMF (2 mL) were added Pd/C (wet, 10%) (50 mg) and Pd(OH)₂/C (wet, 10%) (50 mg) under N₂. The suspension was degassed and purged with H₂ 3 times. The mixture was stirred under H₂ (15 psi) at 20° C. for 48 h. The reaction mixture was filtered under reduced pressure. The residue was purified by reversed phase flash (flow: 30 mL/min; gradient: from 10-70% MeCN in water (0.1% TFA) over 25 min; column: Welch Ultimate XB-C18, 20-40 μm, 100 Å, I.D. 95 mm×H 365 mm) to afford tert-butyl 4-(1-(2,6-dioxopiperidin-3-yl)-3-ethyl-2-oxo-2,3-dihydro-1H-benzo[d]imidazol-5-yl)piperidine-1-carboxylate (5, 0.27 g, 591.41 μmol, 37% yield) as a white solid.

### Step 4; 3-(3-ethyl-2-oxo-5-(piperidin-4-yl)-2,3-dihydro-1H-benzo[d]imidazol-1-yl)piperidine-2,6-dione (6)

A solution of tert-butyl 4-(1-(2,6-dioxopiperidin-3-yl)-3-ethyl-2-oxo-2,3-dihydro-1H-benzo[d]imidazol-5-yl)piperidine-1-carboxylate (5, 600 mg, 1.31 mmol) in HCl/dioxane (2 mL) was stirred at 25° C. for 12 h. The reaction mixture was concentrated under reduced pressure to afford 3-(3-ethyl-2-oxo-5-(piperidin-4-yl)-2,3-dihydro-1H-benzo[d]imidazol-1-yl)piperidine-2,6-dione (6, 468.4 mg, 1.30 mmol, 99% yield) as a white solid. The material was used in the next step directly without purification.

### Step 5: tert-butyl 2-(4-(1-(2,6-dioxopiperidin-3-yl)-3-ethyl-2-oxo-2,3-dihydro-1H-benzo[d]imidazol-5-yl)piperidin-1-yl)acetate (8)

To a solution of 3-(3-ethyl-2-oxo-5-(piperidin-4-yl)-2,3-dihydro-1H-benzo[d]imidazol-1-yl)piperidine-2,6-dione (6, 0.48 g, 1.35 mmol) and DIPEA (435 mg, 3.37 mmol) in DMF (5 mL) was added tert-butyl 2-bromoacetate (7, 315.22 mg, 1.62 mmol). After 2 h, the reaction mixture was quenched with water (5 mL). The brown solid was triturated with petroleum ether:ethyl acetate=1:1(10 mL) for 12 h to afford tert-butyl 2-(4-(1-(2,6-dioxopiperidin-3-yl)-3-ethyl-2-oxo-2,3-dihydro-1H-benzo[d]imidazol-5-yl)piperidin-1-yl)acetate (8, 330 mg, 694.28 μmol, 52% yield) as a white solid. LCMS (ESI): m/z 471.5[M+H]⁺

### Step 6: 2-(4-(1-(2,6-dioxopiperidin-3-yl)-3-ethyl-2-oxo-2,3-dihydro-1H-benzo[d]imidazol-5-yl)piperidin-1-yl)acetic acid (9)

A solution of tert-butyl 2-(4-(1-(2,6-dioxopiperidin-3-yl)-3-ethyl-2-oxo-2,3-dihydro-1H-benzo[d]imidazol-5-yl)piperidin-1-yl)acetate (8, 330 mg, 701.29 μmol) in HCl/dioxane (5 mL) was stirred for 12 h. The mixture was concentrated to afford 2-(4-(1-(2,6-dioxopiperidin-3-yl)-3-ethyl-2-oxo-2,3-dihydro-1H-benzo[d]imidazol-5-yl)piperidin-1-yl)acetic acid (9, 290 mg, 699.72 μmol, 99% yield) as a white solid. The material was used in the next step directly without purification.

### 2-(5-(1-(2,6-dioxopiperidin-3-yl)-3-methyl-2-oxo-2,3-dihydro-1H-benzo[d]imidazol-5-yl)pyridin-2-yl)acetic acid (5)

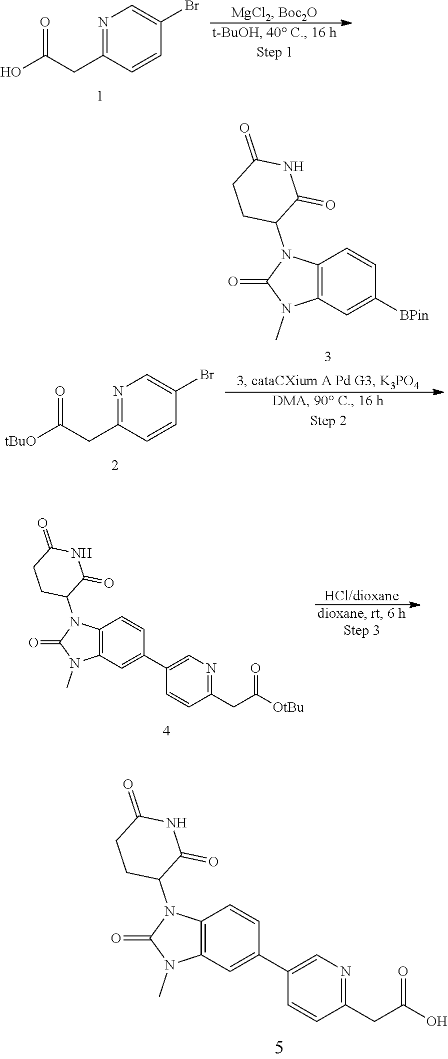

### Step 1: tert-butyl 2-(5-bromopyridin-2-yl)acetate (2)

A mixture of 2-(5-bromopyridin-2-yl)acetic acid (1, 1 g, 4.63 mmol), MgCl₂ (44.07 mg, 462.89 μmol) and Boc₂O (1.31 g, 6.02 mmol, 1.38 mL) in t-BuOH (10 mL) was degassed and purged with N₂ 3 times, and then the mixture was stirred at 40° C. for 16 h. The reaction mixture was poured into water (20 mL) and extracted with ethyl acetate (2×20 mL). Organic phases were combined and washed with brine (30 mL), dried over anhydrous Na₂SO₄, filtered and concentrated under reduced pressure. The residue was purified by flash silica gel chromatography (petroleum ether/ethyl acetate=1/0 to 10/1) to afford tert-butyl 2-(5-bromopyridin-2-yl)acetate (2, 990 mg, 3.60 mmol, 78% yield) as yellow oil. LCMS (ESI): m/z 216.0 [M+H-t-Bu⁺]⁺

### Step 2: tert-butyl 2-(5-(1-(2,6-dioxopiperidin-3-yl)-3-methyl-2-oxo-2,3-dihydro-1H-benzo[d]imidazol-5-yl)pyridin-2-yl)acetate (4)

A mixture of 3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (3, 100 mg, 259.59 μmol), tert-butyl 2-(5-bromopyridin-2-yl)acetate (2, 79.29 mg, 285.55 μmol), cataCXium A Pd G3 (18.93 mg, 25.96 μmol) and K₃PO₄ (165.31 mg, 778.77 μmol) in DMA (1 mL) was degassed and purged with N₂ 3 times. After 16 h at 90° C., the reaction mixture was purified by flash silica gel chromatography (petroleum ether/ethyl acetate=1/0 to 0/1) followed by prep-HPLC (flow: 25 mL/min; gradient: from 82-52% water (0.1% TFA) in MeCN over 40 min; column: Phenomenex Synergi C18 150×25 mm×10 μm) to afford tert-butyl 2-(5-(1-(2,6-dioxopiperidin-3-yl)-3-methyl-2-oxo-2,3-dihydro-1H-benzo[d]imidazol-5-yl)pyridin-2-yl)acetate (4, 26 mg, 45.60 μmol, 18% yield, TFA salt) as a white solid. LCMS (ESI): m/z 451.2 [M+H]^{+ 1}H NMR (400 MHZ, d₆-DMSO) δ 11.13 (s, 1H), 8.90 (d, J=2.0 Hz, 1H), 8.23-8.16 (m, 1H), 7.64 (d, J=1.6 Hz, 1H), 7.51 (d, J=8.0 Hz, 1H), 7.45 (dd, J=1.6, 8.4 Hz, 1H), 7.26 (d, J=8.4 Hz, 1H), 5.43 (dd, J=5.6, 12.8 Hz, 1H), 3.84 (s, 2H), 3.43 (s, 3H), 3.00-2.87 (m, 1H), 2.83-2.73 (m, 1H), 2.68-2.62 (m, 1H), 2.11-2.02 (m, 1H), 1.43 (s, 9H).

### Step 3: 2-(5-(1-(2,6-dioxopiperidin-3-yl)-3-methyl-2-oxo-2,3-dihydro-1H-benzo[d]imidazol-5-yl)pyridin-2-yl)acetic acid (5)

To a solution of tert-butyl 2-(5-(1-(2,6-dioxopiperidin-3-yl)-3-methyl-2-oxo-2,3-dihydro-1H-benzo[d]imidazol-5-yl)pyridin-2-yl)acetate (4, 350 mg, 776.94 μmol) in dioxane (3 mL) was added HCl/dioxane (4 M, 3 mL). After 6 h, the reaction mixture was concentrated under reduced pressure to afford 2-(5-(1-(2,6-dioxopiperidin-3-yl)-3-methyl-2-oxo-2,3-dihydro-1H-benzo[d]imidazol-5-yl)pyridin-2-yl)acetic acid (5, 350 mg, 812.36 μmol, HCl salt) as a white solid. The material was used into the next step without further purification. LCMS (ESI): m/z 395.1 [M+H]⁺

### 2-(4-(3-(2,6-bis(benzyloxy)pyridin-3-yl)-1-methyl-1H-indazol-6-yl)phenyl)acetic acid (4)

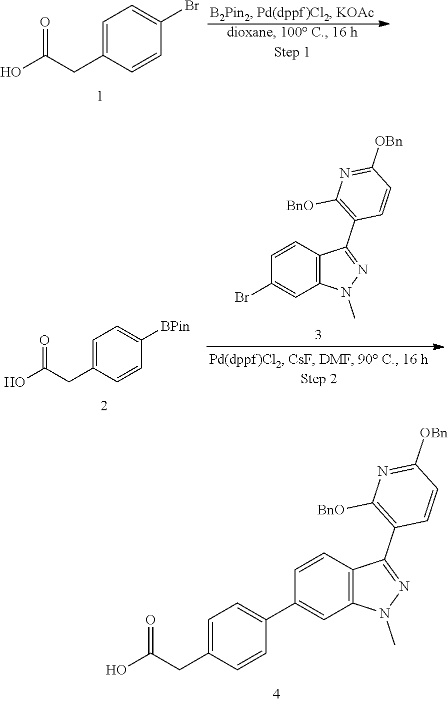

### Step 1: 2-(4-(4,4,5,5-tetramethyl-1,3,2-dioxaborolan-2-yl)phenyl)acetic acid (2)

To a solution of 2-(4-bromophenyl)acetic acid (1, 5.00 g, 23.25 mmol) in 1,4-dioxane (50 mL) was added B₂Pin₂ (8.86 g, 34.88 mmol), KOAc (11.41 g, 116.26 mmol) and Pd(dppf)Cl₂ (850.65 mg, 1.16 mmol). The mixture was stirred at 100° C. for 16 h under N₂. The residue was diluted with H₂O (500 mL) and extracted with ethyl acetate (200 mL×2). The combined organic layers were washed with brine (500 mL×3), dried over Na₂SO₄, filtered and concentrated under reduced pressure. The residue was purified by column chromatography (SiO₂, petroleum ether:ethyl acetate=100:1 to 0:1) to afford 2-(4-(4,4,5,5-tetramethyl-1,3,2-dioxaborolan-2-yl)phenyl)acetic acid (2, 6 g, 22.89 mmol, 98% yield) as a white solid.

¹H NMR (400 MHZ, CDCl3, 298 K) δ (ppm)=7.79 (d, J=7.8 Hz, 2H), 7.30 (d, J=7.8 Hz, 2H), 3.67 (s, 2H), 1.37-1.32 (m, 12H)

### Step 2: 2-(4-(3-(2,6-bis(benzyloxy)pyridin-3-yl)-1-methyl-1H-indazol-6-yl)phenyl)acetic acid (4)

To a solution of 3-(2,6-bis(benzyloxy)pyridin-3-yl)-6-bromo-1-methyl-1H-indazole (3, 1 g, 2.00 mmol), 2-(4-(4,4,5,5-tetramethyl-1,3,2-dioxaborolan-2-yl)phenyl)acetic acid (2, 1.05 g, 4.00 mmol) in DMF (3 mL) were added CsF (910.72 mg, 6.00 mmol) and Pd(dppf)Cl₂ (73.11 mg, 99.92 μmol). The mixture was stirred at 90° C. for 16 h under N₂. The residue was diluted with H₂O (100 mL) and extracted with ethyl acetate (100 mL×2). The combined organic layers were washed with brine (200 mL×3), dried over Na₂SO₄, filtered and concentrated under reduced pressure. The residue was purified by column chromatography (SiO₂, petroleum ether:ethyl acetate=100:1 to 1:1) to afford 2-(4-(3-(2,6-bis(benzyloxy)pyridin-3-yl)-1-methyl-1H-indazol-6-yl)phenyl)acetic acid (4, 420 mg, 755.91 μmol, 38% yield) as a white solid. LCMS (ESI): m/z 556.2 [M+H]⁺

### (R)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetic acid (7)

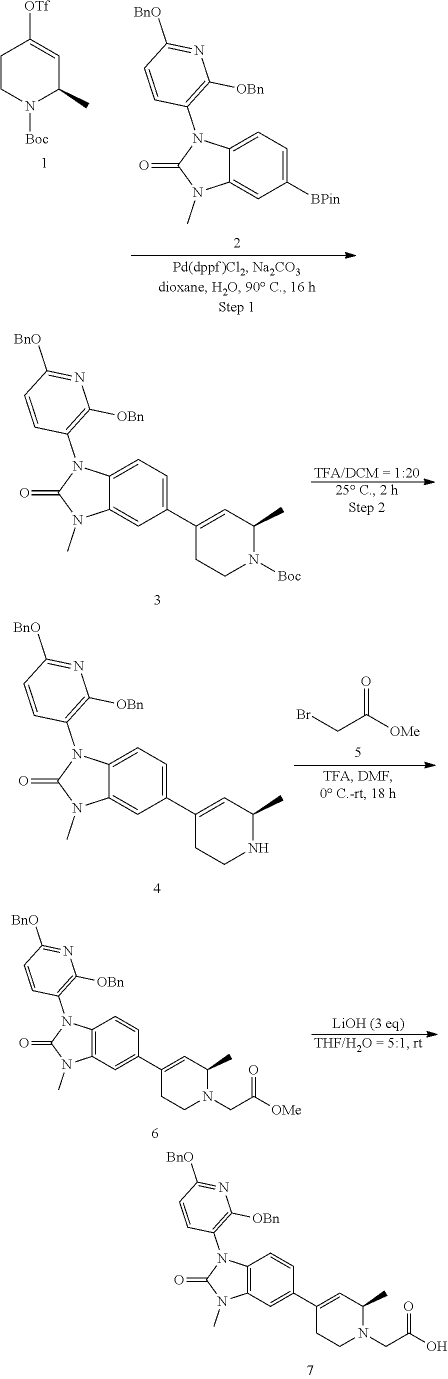

### Step 1: (R)-tert-butyl 4-(1-(2,6-bis(benzyloxy)pyridin-3-yl)-3-methyl-2-oxo-2,3-dihydro-1H-benzo[d]imidazol-5-yl)-2-methyl-5,6-dihydropyridine-1(2H)-carboxylate (3)

To a solution of (R)-tert-butyl 2-methyl-4-(((trifluoromethyl)sulfonyl)oxy)-5,6-dihydropyridine-1(2H)-carboxylate (1, 600 mg, 1.74 mmol) and 1-(2,6-bis(benzyloxy)pyridin-3-yl)-3-methyl-5-(4,4,5,5-tetramethyl-1,3,2-dioxaborolan-2-yl)-1H-benzo[d]imidazol-2(3H)-one (2, 780 mg, 1.38 mmol) in dioxane (15 mL) were added Na₂CO₃ (2 M in H₂O, 2.61 mL) and Pd(dppf)Cl₂ (63.6 mg, 86.9 μmol). The mixture was stirred at 90° C. under N₂ for 16 h. The mixture was concentrated under reduced pressure. The residue was diluted with EtOAc (50 mL) and water (50 mL). The layer was separated and the aqueous phase was extracted with EtOAc (3×50 mL). The combined organic layers were dried over Na₂SO₄, filtered and concentrated. The residue was purified by flash silica gel chromatography (30-40% ethyl acetate in petroleum ether) to afford (R)-tert-butyl 4-(1-(2,6-bis(benzyloxy)pyridin-3-yl)-3-methyl-2-oxo-2,3-dihydro-1H-benzo[d]imidazol-5-yl)-2-methyl-5,6-dihydropyridine-1(2H)-carboxylate (3, 700 mg, 1.04 mmol, 60% yield) as a yellow solid. LCMS (ESI): m/z 633.3 [M+H]⁺

¹H NMR (400 MHZ, DMSO-d₆) δ=7.86-7.71 (m, 1H), 7.48-7.23 (m, 10H), 7.06 (d, J=8.4 Hz, 1H), 6.72-6.52 (m, 2H), 6.08 (d, J=13.6 Hz, 1H), 5.46-5.27 (m, 4H), 4.52 (d, J=5.2 Hz, 1H), 4.22 (s, 1H), 3.94 (s, 1H), 3.73-3.60 (m, 1H), 3.40) (s, 3H), 3.03-2.85 (m, 1H), 2.78-2.65 (m, 1H), 2.44 (s, 1H), 2.34 (d, J=16.0 Hz, 1H), 1.99 (d, J=1.6 Hz, 1H), 1.43 (s, 9H), 1.07 (s, 6H).

### Step 2: (R)-1-(2,6-bis(benzyloxy)pyridin-3-yl)-3-methyl-5-(6-methyl-1,2,3,6-tetrahydropyridin-4-yl)-1H-benzo[d]imidazol-2(3H)-one (4)

A solution of (R)-tert-butyl 4-(1-(2,6-bis(benzyloxy)pyridin-3-yl)-3-methyl-2-oxo-2,3-dihydro-1H-benzo[d]imidazol-5-yl)-2-methyl-5,6-dihydropyridine-1(2H)-carboxylate (3, 700 mg, 1.04 mmol) in DCM (20 mL) and TFA (1 mL). After 2 h, the mixture was concentrated under reduced pressure to give (R)-1-(2,6-bis(benzyloxy)pyridin-3-yl)-3-methyl-5-(6-methyl-1,2,3,6-tetrahydropyridin-4-yl)-1H-benzo[d]imidazol-2(3H)-one (4, 400 mg, 573.74 μmol, 52% yield, TFA salt) as a yellow solid. The material was used in the next step without further purification. LCMS (ESI): m/z 533.4 [M+H]⁺

### Step 3: (R)-methyl 2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetate (6)

To a solution of (R)-1-(2,6-bis(benzyloxy)pyridin-3-yl)-3-methyl-5-(6-methyl-1,2,3,6-tetrahydropyridin-4-yl)-1H-benzo[d]imidazol-2(3H)-one (4, 400 mg, 751 μmol) in DMF (10 mL) was added TEA (380) mg, 3.75 mmol) at (° C.) The mixture was stirred at 25° C. for 15 min before methyl 2-bromoacetate (5, 172.32 mg, 1.13 mmol) was added to the mixture. After 16 h, the solvent was removed and the residue was diluted with EtOAc (50 mL) and water (40 mL). The layers were separated and the aqueous phase was extracted with EtOAc (3×40 mL). The combined organic layers were dried over Na₂SO₄, filtered and concentrated. The residue was purified by flash silica gel chromatography (30-40% ethyl acetate in petroleum ether) to give (R)-methyl 2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetate (6, 350 mg, 550 μmol, 73% yield) as a yellow solid. LCMS (ES+): m/z 605.2 [M+H]⁺

¹H NMR (400 MHZ, DMSO-d₆) δ=7.95 (s, 1H), 7.80 (d, J=8.4 Hz, 1H), 7.48-7.42 (m, 3H), 7.41-7.33 (m, 4H), 7.30-7.23 (m, 8H), 7.07-7.03 (m, 1H), 6.68-6.56 (m, 3H), 6.10-5.94 (m, 1H), 5.43-5.29 (m, 6H), 3.63 (d, J=2.0 Hz, 4H), 3.43-3.38 (m, 5H), 3.17 (s, 1H), 2.89 (s, 3H), 2.73 (s, 3H).

### Step 4: (R)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetic acid (7)

To a solution of (R)-methyl 2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetate (6, 300 mg, 496 μmol) in THF (10 mL) and H₂O (2 mL) was added LiOH (59.41 mg, 2.48 mmol). After 2 h, the pH of the reaction mixture was acidified to 7 with IN HCl. The reaction was diluted with EtOAc (50 mL) and water (50 mL). The layer was separated, and the aqueous phase was extracted with EtOAc (3×50 mL). The combined organic layers were dried over Na₂SO₄, filtered and concentrated to give (R)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetic acid (7, 300 mg, 472.35 μmol, 95% yield) as a yellow solid. LCMS (ES+): m/z 591.3 [M+H]⁺

### 2-((trans)-4-(3-(2,6-bis(benzyloxy)pyridin-3-yl)-1-methyl-1H-indazol-6-yl)cyclohexyl)acetic acid (7a, first eluting isomer, arbitrary assignment) and 2-((cis)-4-(3-(2,6-bis(benzyloxy)pyridin-3-yl)-1-methyl-1H-indazol-6-yl)cyclohexyl)acetic acid (7b, Second Eluting Isomer, Arbitrary Assignment)

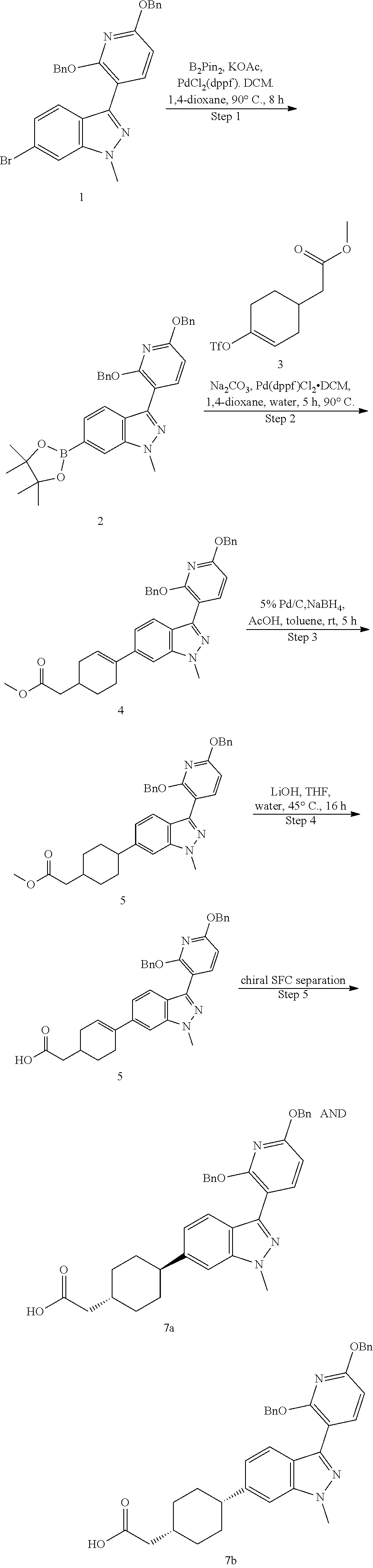

### Step 1: 3-(2,6-dibenzyloxy-3-pyridyl)-1-methyl-6-(4,4,5,5-tetramethyl-1,3,2-dioxaborolan-2-yl)indazole (2)

Into a 50 mL pressure tube containing a well-stirred solution of 6-bromo-3-(2,6-dibenzyloxy-3-pyridyl)-1-methyl-indazole (1, 2 g, 4.00 mmol) in 1,4-dioxane (20 mL) were added bis(pinacolato)diboron (1.12 g, 4.40 mmol) and potassium acetate (1.18 g, 11.99 mmol). The resulting suspension was degassed by bubbling nitrogen gas through for 10 min. Then Pd(dppf)Cl₂·DCM (326.40 mg, 399.69 μmol) was added and degassed for additional 5 min. The reaction mixture was stirred at 90° C. for 8 h. The reaction mixture was filtered through Celite and washed with ethyl acetate (100 mL). The filtrate was concentrated under reduced pressure and purified by flash silica gel column chromatography (20% ethyl acetate in pet ether) to afford 3-(2,6-dibenzyloxy-3-pyridyl)-1-methyl-6-(4,4,5,5-tetramethyl-1,3,2-dioxaborolan-2-yl)indazole (2, 2 g, 3.49 mmol, 87% yield) as a gummy yellow liquid. LCMS (ES+): m/z 548.2 [M+H]⁺

### Step 2: Methyl 2-[4-[3-(2,6-dibenzyloxy-3-pyridyl)-1-methyl-indazol-6-yl]cyclohex-3-en-1-yl]acetate (4)

Into a 25 mL pressure tube containing a well-stirred solution of 3-(2,6-dibenzyloxy-3-pyridyl)-1-methyl-6-(4,4,5,5-tetramethyl-1,3,2-dioxaborolan-2-yl)indazole (2, 2 g, 3.52 mmol) and methyl 2-[4-(trifluoromethylsulfonyloxy)cyclohex-3-en-1-yl]acetate (3, 2.00 g, 5.96 mmol) in 1,4-dioxane (27 mL) and water (2 mL) was added sodium carbonate (1.12 g, 10.56 mmol). The suspension was degassed by bubbling nitrogen gas through for 10 min. Then, Pd(dppf)Cl₂·DCM (431.31 mg, 528.16 μmol) was added and degassed for additional 5 min. After 5 h at 90° C., the reaction mixture was filtered through Celite and washed with ethyl acetate (100 mL). The filtrate was concentrated under reduced pressure and purified by flash silica gel column chromatography (25% ethyl acetate in pet ether) to afford methyl 2-[4-[3-(2,6-dibenzyloxy-3-pyridyl)-1-methyl-indazol-6-yl]cyclohex-3-en-1-yl]acetate (4, 1.2 g, 1.97 mmol, 56% yield) as a gummy yellow liquid. LCMS (ES+): m/z 574.2 [M+H]⁺

### Step 3: Methyl 2-[4-[3-(2,6-dibenzyloxy-3-pyridyl)-1-methyl-indazol-6-yl]cyclohexyl]acetate (5)

Into a 50 mL single neck round bottom flask containing a well-stirred solution of methyl 2-[4-[3-(2,6-dibenzyloxy-3-pyridyl)-1-methyl-indazol-6-yl]cyclohex-3-en-1-yl]acetate (4, 1.4 g, 2.09 mmol) in toluene (50 mL) were added acetic acid (752.68 mg, 12.53 mmol, 716.84 μL), palladium on carbon (5 wt. %) (350 mg, 2.09 mmol, 5% purity) followed by sodium borohydride (474.19 mg, 12.53 mmol) in 2 portions over a period of 5 min. After 2 h, additional sodium borohydride (474.19 mg, 12.53 mmol) was added in 2 portions. After 5 h the reaction mixture was filtered through Celite and washed with a mixture of THF (200 mL) and toluene (50 mL), followed by EtOAc (100 mL). The filtrate was concentrated under reduced pressure and purified by flash silica gel column chromatography (33% ethyl acetate in pet ether) to afford methyl 2-[4-[3-(2,6-dibenzyloxy-3-pyridyl)-1-methyl-indazol-6-yl]cyclohexyl]acetate (5, 1.1 g, 1.78 mmol, 85% yield) as a colorless gummy solid. LCMS (ES+): m/z 576.2 [M+H]⁺

### Step 4: 2-[4-[3-(2,6-dibenzyloxy-3-pyridyl)-1-methyl-indazol-6-yl]cyclohexyl]acetic acid (6)

Into a 100 mL single neck round bottom flask containing well-stirred solution of methyl 2-[4-[3-(2,6-dibenzyloxy-3-pyridyl)-1-methyl-indazol-6-yl]cyclohexyl]acetate (5, 1.1 g, 1.78 mmol) in THF (20 mL) was added a solution of lithium hydroxide monohydrate (1.49 g, 35.55 mmol) in water (6 mL). The resulting solution was stirred at 45° C. for 16 h. The reaction mixture was concentrated under reduced pressure and acidified with 1.5 N HCl solution in water (5 mL). The solid was filtered and washed with methyl tert-butyl ether (25 mL) to afford 2-(4-(3-(2,6-bis(benzyloxy)pyridin-3-yl)-1-methyl-1H-indazol-6-yl)cyclohexyl)acetic acid (6, 1 g, 1.67 mmol, 94% yield) as an off-white solid. The material was used in the next step without further purification. LCMS (ES+): m/z 562.2 [M+H]⁺

### Step 5: 2-((trans)-4-(3-(2,6-bis(benzyloxy)pyridin-3-yl)-1-methyl-1H-indazol-6-yl)cyclohexyl)acetic acid (7a, first eluting isomer) and 2-((cis)-4-(3-(2,6-bis(benzyloxy)pyridin-3-yl)-1-methyl-1H-indazol-6-yl)cyclohexyl)acetic acid (7b, Second Eluting Isomer)

The isomers were separated by chiral SFC: Method details: Column-Chiralpak OX—H: Co-Solvent: 0.5% isopropyl amine in methanol: Flow Rate: 5 mL/min. Outlet Pressure: 100 bar. First eluted isomer (arbitrarily assigned as trans-isomer): 2-((trans)-4-(3-(2,6-bis(benzyloxy)pyridin-3-yl)-1-methyl-1H-indazol-6-yl)cyclohexyl)acetic acid (7a, 500 mg, 835.37 μmol, 47% yield) (RT 4.0 min, chiral purity 97.07%), as an off-white solid. LCMS (ES+): m/z 562.2 [M+H]⁺

Second eluted isomer (arbitrarily assigned as cis-isomer): 2-((cis)-4-(3-(2,6-bis(benzyloxy)pyridin-3-yl)-1-methyl-1H-indazol-6-yl)cyclohexyl)acetic acid (7b, 400 mg, 672.64 μmol, 38% yield) (RT 4.97 min, chiral purity 100%), as an off-white solid. LCMS (ES+): m/z 562.2 [M+H]⁺

### 2-[4-[1-(2,6-dioxo-3-piperidyl)-3-methyl-2-oxo-benzimidazol-5-yl]-3-fluoro-1-piperidyl]acetic acid (10)

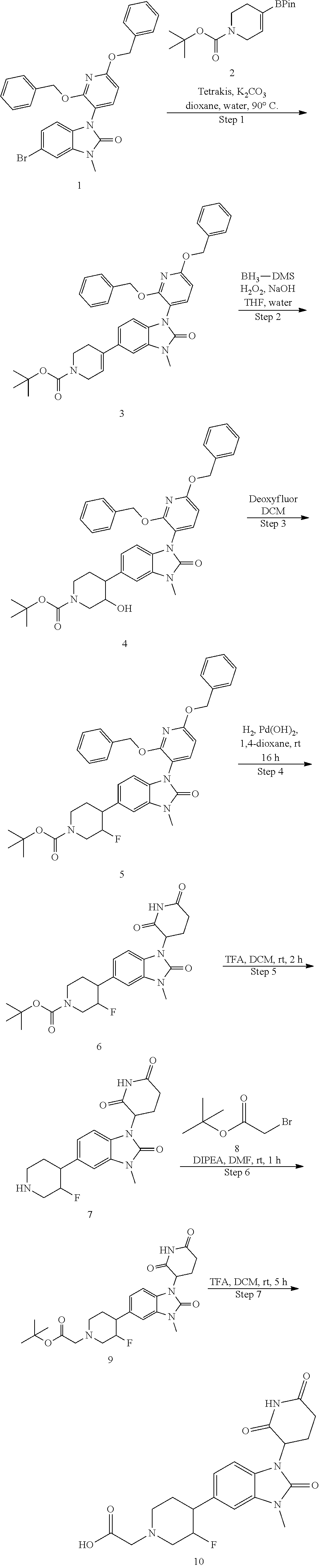

### Step 1: tert-butyl 4-[1-(2,6-dibenzyloxy-3-pyridyl)-3-methyl-2-oxo-benzimidazol-5-yl]-3,6-dihydro-2H-pyridine-1-carboxylate (3)

To a mixture of 5-bromo-1-(2,6-dibenzyloxy-3-pyridyl)-3-methyl-benzimidazol-2-one (1, 20 g, 38.73 mmol) and tert-butyl 4-(4,4,5,5-tetramethyl-1,3,2-dioxaborolan-2-yl)-3,6-dihydro-2H-pyridine-1-carboxylate (2, 15.57 g, 50.35 mmol) in 1,4-dioxane (160 mL) and Water (40 mL) was added Potassium carbonate (16.06 g, 116.19 mmol). The resulting mixture was purged with argon gas for 30 min. Tetrakis (4.48 g, 3.87 mmol) was added and the mixture was heated to reflux at 90° C.) for 12 h. The mixture was filtered through Celite and concentrated and water (200 mL) was added. The mixture was extracted with dichloromethane (3×200 mL), concentrated and purified via silica gel column chromatography (DP was eluted at 25% ethyl acetate in Pet ether) to afford tert-butyl 4-[1-(2,6-dibenzyloxy-3-pyridyl)-3-methyl-2-oxo-benzimidazol-5-yl]-3,6-dihydro-2H-pyridine-1-carboxylate (3, 18.0 g, 27.93 mmol, 72% yield) as a brown viscous material. LCMS (ES+): m/z 619.5 [M+H]+

### Step 2: tert-butyl 4-[1-(2,6-dibenzyloxy-3-pyridyl)-3-methyl-2-oxo-benzimidazol-5-yl]-3-hydroxy-piperidine-1-carboxylate (4)

To a stirred a solution of tert-butyl 4-[1-(2,6-dibenzyloxy-3-pyridyl)-3-methyl-2-oxo-benzimidazol-5-yl]-3,6-dihydro-2H-pyridine-1-carboxylate (3, 18 g, 29.09 mmol) in THF (1800 mL) was added Borane dimethyl sulfide complex (11.05 g, 145.45 mmol, 13.80 mL) at (° C.) under argon atmosphere. After 12 h. water (200 mL) was added at (° C.) dropwise, followed by the addition of sodium hydroxide (IN) (5.82 g, 145.46 mmol) dissolved in Water (200 mL). The resulting mixture was stirred at 0° C. for 20 min. Hydrogen peroxide (35%) (4.95 g, 145.46 mmol, 4.50 mL) was added and the resulting mixture was stirred at RT for 4 h. The reaction mixture was diluted with water (250 mL) and extracted with DCM (200 mL×3). The combined organic layer was dried under high vacuum. The material was purified by column chromatography (ethyl acetate in Petroleum ether) to afford tert-butyl 4-[1-(2,6-dibenzyloxy-3-pyridyl)-3-methyl-2-oxo-benzimidazol-5-yl]-3-hydroxy-piperidine-1-carboxylate (4, 15.5 g, 23.86 mmol, 82% yield) as a off white solid. LCMS (ES+): m/z 659.4 [M+Na]⁺

### Step 3: tert-butyl 4-[1-(2,6-dibenzyloxy-3-pyridyl)-3-methyl-2-oxo-benzimidazol-5-yl]-3-fluoro-piperidine-1-carboxylate (5)

To a stirred solution of tert-butyl 4-[1-(2,6-dibenzyloxy-3-pyridyl)-3-methyl-2-oxo-benzimidazol-5-yl]-3-hydroxy-piperidine-1-carboxylate (4, 15.5 g, 24.34 mmol) in DCM (800 mL) was added Deoxyflour (10.77 g, 48.69 mmol, 8.98 mL) at −70° C. After 3 h, the mixture was diluted with water (200 mL) and quenched with saturated sodium bicarbonate solution (200 mL) and extracted with ethyl acetate (3×150 mL). The combined organic layers were dried under high vacuum, and the residue purified by column chromatography (ethyl acetate in pet ether) to afford tert-butyl 4-[1-(2,6-dibenzyloxy-3-pyridyl)-3-methyl-2-oxo-benzimidazol-5-yl]-3-fluoro-piperidine-1-carboxylate (11 g, 9.84 mmol, 40% yield) as a viscous oil. LCMS (ES+): m/z 639.6[M+H]⁺

### Step 4: tert-butyl 4-[1-(2,6-dioxo-3-piperidyl)-3-methyl-2-oxo-benzimidazol-5-yl]-3-fluoro-piperidine-1-carboxylate (6)

Into a 100 mL single neck round bottom flask containing a well-stirred solution of tert-butyl 4-[1-(2,6-dibenzyloxy-3-pyridyl)-3-methyl-2-oxo-benzimidazol-5-yl]-3-fluoro-piperidine-1-carboxylate (5, 700 mg, 1.10 mmol) in anhydrous 1,4-dioxane (12 mL) was added palladium hydroxide on carbon (560.00 mg, 797.52 μmol, 20% purity). After 16 h, the reaction mixture was filtered through Celite, washing with 1,4-dioxane (100 mL). The filtrate was concentrated under reduced pressure to afford tert-butyl 4-[1-(2,6-dioxo-3-piperidyl)-3-methyl-2-oxo-benzimidazol-5-yl]-3-fluoro-piperidine-1-carboxylate (6, 470 mg, 994.41 μmol, 91% yield,) as a colorless solid. The material was used in the next step without further purification. LCMS (ES+): m/z 405.2 [M-tBu+H]⁺

### Step 5: 3-[5-(3-fluoro-4-piperidyl)-3-methyl-2-oxo-benzimidazol-1-yl]piperidine-2,6-dione (7)

Into a 100 mL single neck round bottom flask containing well-stirred solution of tert-butyl 4-[1-(2,6-dioxo-3-piperidyl)-3-methyl-2-oxo-benzimidazol-5-yl]-3-fluoro-piperidine-1-carboxylate (6, 470 mg, 994.41 μmol) in anhydrous DCM (10 mL) was added TFA (1.48 g, 12.98 mmol, 1 mL). After 2 h the volatiles were removed under reduced pressure. The residue was triturated with MTBE (50 mL), filtered and dried to obtain 3-[5-(3-fluoro-4-piperidyl)-3-methyl-2-oxo-benzimidazol-1-yl]piperidine-2,6-dione (7, 420 mg, 865.13 μmol, 87% yield, TFA salt) as a colorless solid. LCMS (ES+): m/z 361.1 [M+H]⁺

### Step 6: tert-butyl 2-[4-[1-(2,6-dioxo-3-piperidyl)-3-methyl-2-oxo-benzimidazol-5-yl]-3-fluoro-1-piperidyl]acetate (9)

Into a 25 mL single neck round bottom flask containing a well-stirred solution of 3-[5-(3-fluoro-4-piperidyl)-3-methyl-2-oxo-benzimidazol-1-yl]piperidine-2,6-dione (7, 200 mg, 404.72 μmol, TFA salt) in anhydrous DMF (3 mL) were added DIPEA (156.92 mg, 1.21 mmol, 211.48 μL) and tert-butyl 2-bromoacetate (8, 78.94 mg, 404.72 μmol, 59.35 μL) at (° C.) The reaction mixture was stirred at RT for 1 h. The solvent was removed from the reaction mixture and water (30 mL) was added. The solid was filtered and dried under reduced pressure to afford tert-butyl 2-[4-[1-(2,6-dioxo-3-piperidyl)-3-methyl-2-oxo-benzimidazol-5-yl]-3-fluoro-1-piperidyl]acetate (9, 150 mg, 314.87 μmol, 78% yield) as a colorless solid. The material was used in the next step without further purification. LCMS (ES+): m/z 475.2 [M+H]⁺

### Step 7: 2-[4-[1-(2,6-dioxo-3-piperidyl)-3-methyl-2-oxo-benzimidazol-5-yl]-3-fluoro-1-piperidyl]acetic acid (10)

Into a 50 mL single neck round bottom flask containing a well-stirred solution of tert-butyl 2-[4-[1-(2,6-dioxo-3-piperidyl)-3-methyl-2-oxo-benzimidazol-5-yl]-3-fluoro-1-piperidyl]acetate (9, 140 mg, 292.08 μmol) in anhydrous DCM (5 mL) was added TFA (2.96 g, 25.96 mmol, 2.00 mL). After 5 h, the volatiles were removed under reduced pressure and the residue was triturated with MTBE (50 mL), filtered and dried to afford 2-[4-[1-(2,6-dioxo-3-piperidyl)-3-methyl-2-oxo-benzimidazol-5-yl]-3-fluoro-1-piperidyl]acetic acid (10, 135 mg, 247.87 μmol, 85% yield, TFA salt) as an off-white solid. LCMS (ES+): m/z 419.2 [M+H]⁺

### (2R)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (7)

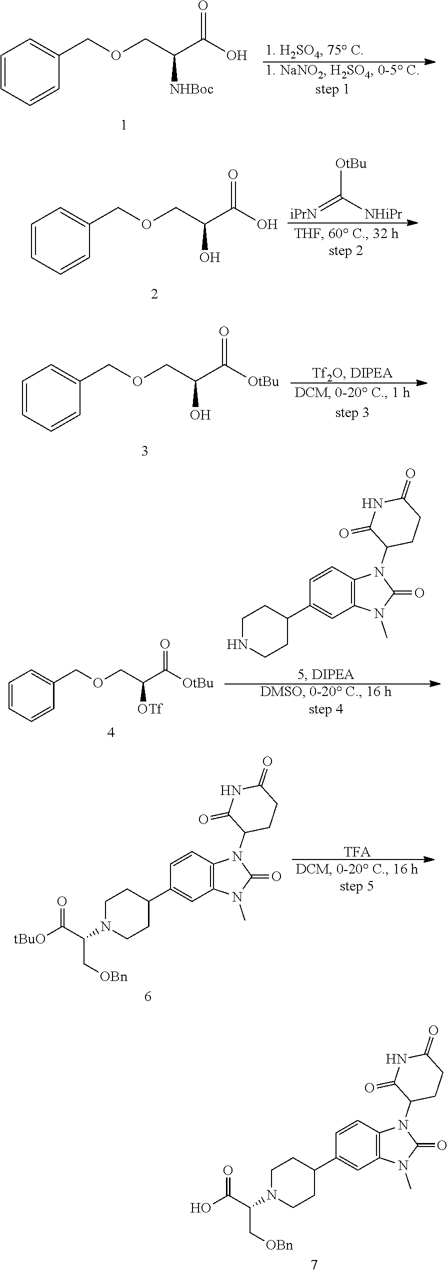

### Step 1: (S)-3-(benzyloxy)-2-hydroxypropanoic acid (2)

A mixture of (S)-3-(benzyloxy)-2-((tert-butoxycarbonyl)amino)propanoic acid (1, 20 g, 67.72 mmol) in H₂SO₄ (2 M, 80.00 mL) was heated at 75° C. for 1 h. The mixture was cooled to 0° C. Sodium nitrite (9.34 g, 135.44 mmol) in H₂O (80 mL) was added dropwise, keeping the reaction mixture at 0-5° C. The mixture was stirred at 20° C. for 12 h. The reaction mixture was extracted with EtOAc (100 mL×3). The combined organic layers were washed with brine (30 mL×2), dried over Na₂SO₄, filtered and concentrated under reduced pressure to afford (S)-3-(benzyloxy)-2-hydroxypropanoic acid (2, 10 g, 50.97 mmol, 75% yield) as yellow oil. The material was used in the next step without purification.

### Step 2: (S)-tert-butyl 3-(benzyloxy)-2-hydroxypropanoate (3)

A mixture of (S)-3-(benzyloxy)-2-hydroxypropanoic acid (2, 3 g, 15.29 mmol) and tert-butyl N,N′-diisopropylcarbamimidate (15.32 g, 76.45 mmol) in THF (20 mL) was heated to 60° C. for 32 h. The mixture was concentrated and the residue was purified by column chromatography (SiO₂, Petroleum ether/Ethyl acetate=100/1 to 5/1) to afford (S)-tert-butyl 3-(benzyloxy)-2-hydroxypropanoate (3, 1.6 g, 6.34 mmol, 41% yield) as yellow oil.

### Step 3: (S)-tert-butyl 3-(benzyloxy)-2-(((trifluoromethyl)sulfonyl)oxy)propanoate (4)

To a mixture of (S)-tert-butyl 3-(benzyloxy)-2-hydroxypropanoate (3, 500 mg, 1.98 mmol) and DIPEA (640.30 mg, 4.95 mmol, 862.93 μL) in DCM (10 mL) was added Tf₂O (585.22 mg, 2.38 mmol) at 0° C.) After 2 h at RT, the mixture was poured into water (30 mL) and extracted with DCM (10 mL). The organic phase was washed with brine (30 mL), dried with anhydrous Na₂SO₄, filtered and concentrated to afford (S)-tert-butyl 3-(benzyloxy)-2-(((trifluoromethyl)sulfonyl)oxy)propanoate (4, 761 mg, 1.98 mmol, 100% yield) as yellow oil. The material was used in the next step without further purification.

### Step 4: (2R)-tert-butyl 3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoate (6)

To a solution of 3-(3-methyl-2-oxo-5-(piperidin-4-yl)-2,3-dihydro-1H-benzo[d]imidazol-1-yl)piperidine-2,6-dione (5, 500 mg, 1.46 mmol) in DMSO (10 mL) was added DIPEA (566.19 mg, 4.38 mmol, 763.06 μL) and (S)-tert-butyl 3-(benzyloxy)-2-(((trifluoromethyl)sulfonyl)oxy)propanoate (4, 561.30 mg, 1.46 mmol) at 0° C.) After 6 h at RT, the mixture was poured into water (30 mL) and extracted with EtOAc (10 mL). The organic phase was washed with brine (30 mL), dried with anhydrous Na₂SO₄, filtered and concentrated. The residue was purified by column chromatography (SiO₂, Petroleum ether/Ethyl acetate=100/1 to 1/2) to afford (2R)-tert-butyl 3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoate (6, 150 mg, 234.10 μmol, 16% yield) as a white solid. LCMS (ESI): m/z 577.1 [M+H]⁺

### Step 5: (2R)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (7)

To a solution of (2R)-tert-butyl 3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoate (6, 150) mg, 260.11 μmol) in DCM (4 mL) was added TFA (2.96 g, 25.96 mmol, 2 mL) at 0° C. After 16 h at RT, the mixture was concentrated to afford (2R)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (7, 165 mg, 260.01 μmol, 100% yield, TFA salt) as yellow oil. The crude product was used in the next step without purification. LCMS (ESI): m/z 521.3 [M+H]⁺

### (2S)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (7)

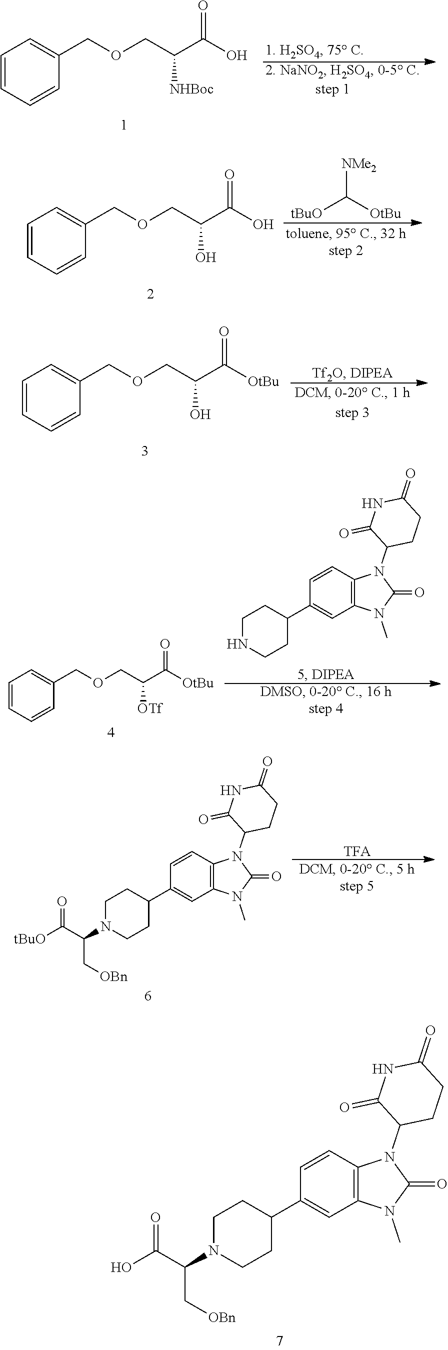

### Step 1: (R)-3-(benzyloxy)-2-hydroxypropanoic acid (2)

A mixture of (R)-3-(benzyloxy)-2-((tert-butoxycarbonyl)amino)propanoic acid (1, 10 g, 33.86 mmol) in H₂SO₄ (2 M, 40.00 mL) was heated at 75° C. for 1 h. The mixture was cooled to 0° C. and sodium nitrite (4.67 g, 67.72 mmol, 2 eq) in H₂O (40 mL) was added dropwise, keeping the reaction mixture at 0-5° C. After 12 h at RT, the mixture was poured into water (100 mL) and extracted with EtOAc (50 mL). The organic phase was washed with brine (100 mL), dried with anhydrous Na₂SO₄, filtered and concentrated to afford (R)-3-(benzyloxy)-2-hydroxypropanoic acid (2, 5 g, 25.48 mmol, 75% yield) as a yellow oil. The crude product was used in the next step without purification. LCMS (ESI): m/z 197.1 [M+H]⁺

¹H NMR (400 MHZ, DMSO-d₆) δ 7.40-7.22 (m, 5H), 4.56-4.46 (m, 2H), 4.16 (t, J=4.4 Hz, 1H), 3.62 (d, J=4.4 Hz, 2H).

### Step 2: (R)-tert-butyl 3-(benzyloxy)-2-hydroxypropanoate (3)

A mixture of (R)-3-(benzyloxy)-2-hydroxypropanoic acid (2, 3 g, 15.29 mmol) and 1,1-di-tert-butoxy-N,N-dimethylmethanamine (12.44 g, 61.16 mmol) in toluene (20 mL) was heated to 90° C. for 32 h. The solvent was removed and the residue was purified by column chromatography (SiO₂, Petroleum ether/Ethyl acetate=100/1 to 5/1) to give (R)-tert-butyl 3-(benzyloxy)-2-hydroxypropanoate (3, 1.5 g, 5.47 mmol, 36% yield) as a yellow oil.

### Step 3: (R)-tert-butyl 3-(benzyloxy)-2-(((trifluoromethyl)sulfonyl)oxy)propanoate (4)

To a mixture of (R)-tert-butyl 3-(benzyloxy)-2-hydroxypropanoate (3, 500 mg, 1.98 mmol) and DIPEA (640.30 mg, 4.95 mmol, 862.93 μL) in DCM (10 mL) was added Tf₂O (585.22 mg, 2.38 mmol) at 0° C. After 2 h at RT, the mixture was poured into water (30 mL) and extracted with DCM (10 mL). The organic phase was washed with brine (30 mL), dried with anhydrous Na₂SO₄, filtered and concentrated to afford (R)-tert-butyl 3-(benzyloxy)-2-(((trifluoromethyl)sulfonyl)oxy)propanoate (4, 761 mg, 1.98 mmol, 100% yield) as yellow oil. The material was used in the next step without further purification.

### Step 4: (2S)-tert-butyl 3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoate (6)

To a solution of 3-(3-methyl-2-oxo-5-(piperidin-4-yl)-2,3-dihydro-1H-benzo[d]imidazol-1-yl)piperidine-2,6-dione (5, 500 mg, 1.46 mmol) in DMSO (10 mL) was added DIPEA (566.19 mg, 4.38 mmol, 763.06 μL) and (R)-tert-butyl 3-(benzyloxy)-2-(((trifluoromethyl)sulfonyl)oxy)propanoate (4, 561.30 mg, 1.46 mmol) at (° C.) After 16 h at RT, the mixture was poured into water (30 mL) and extracted with EtOAc (10 mL). The organic phase was washed with brine (30 mL), dried with anhydrous Na₂SO₄, filtered and concentrated. The residue was purified by column chromatography (SiO₂, Petroleum ether/Ethyl acetate=100/1 to 1/2) to afford (2S)-tert-butyl 3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoate (6, 190 mg, 322.88 μmol, 22% yield) as white solid. LCMS (ESI): m/z 577.3 [M+H]⁺

### Step 5: (2S)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (7)

To a solution of (2S)-tert-butyl 3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoate (6, 120 mg, 208.09 μmol) in DCM (4 mL) was added TFA (2.96 g, 25.96 mmol, 2 mL) at 0° C.) the mixture was stirred at RT for 16 h. The mixture was concentrated to afford (28)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (7. 132 mg, 208.01 μmol, 100% yield, TFA salt) as yellow oil. The material was used in the next step without further purification. LCMS (ESI): m/z 521.2 [M+H]⁺

### 2-(4-((3-(2,6-bis(benzyloxy)pyridin-3-yl)-1-methyl-1H-indazol-6-yl)amino)-3-methylphenyl)acetic acid (4)

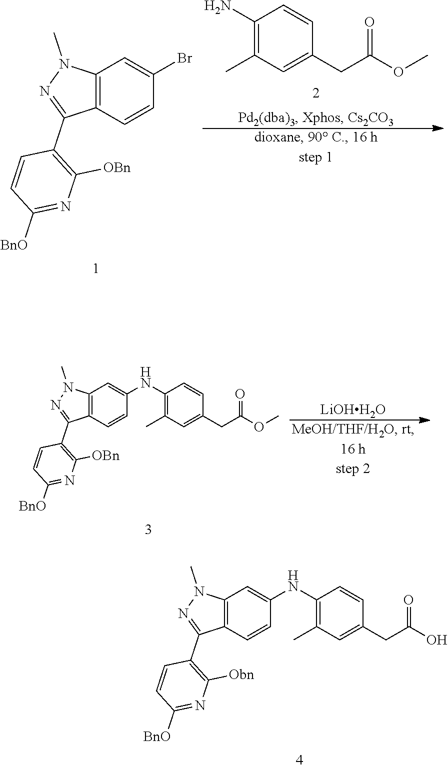

### Step 1: methyl 2-(4-((3-(2,6-bis(benzyloxy)pyridin-3-yl)-1-methyl-1H-indazol-6-yl)amino)-3-methylphenyl)acetate (3)

To a mixture of 3-(2,6-bis(benzyloxy)pyridin-3-yl)-6-bromo-1-methyl-1H-indazole (1, 500 mg, 999.23 μmol) and methyl 2-(4-amino-3-methylphenyl)acetate (2, 214.89 mg, 1.20 mmol) in dioxane (10 mL) were added Cs₂CO₃ (651.14 mg, 2.00 mmol), Xphos (23.82 mg, 49.96 μmol) and Pd₂(dba)₃ (45.75 mg, 49.96 μmol). The mixture was stirred at 90° C. for 16 h under N₂. The mixture was concentrated under reduced pressure and purified by column chromatography (SiO₂, petroleum ether/ethyl acetate=10/0 to 1/1; petroleum ether/ethyl acetate=3/1, R_{f}=0.3) to afford 2-(4-((3-(2,6-bis(benzyloxy)pyridin-3-yl)-1-methyl-1H-indazol-6-yl)amino)-3-methyl methylphenyl)acetate (3, 0.5 g, 835.16 μmol, 84% yield) as a yellow oil. LCMS (ESI): m/z 599.2 [M+H]⁺

### Step 2: 2-(4-((3-(2,6-bis(benzyloxy)pyridin-3-yl)-1-methyl-1H-indazol-6-yl)amino)-3-methylphenyl)acetic acid (4)

To a solution of methyl methyl 2-(4-((3-(2,6-bis(benzyloxy)pyridin-3-yl)-1-methyl-1H-indazol-6-yl)amino)-3-methylphenyl)acetate (3, 400 mg, 668.13 μmol) in THF (4 mL), water (4 mL) and methanol (4 mL) was added LiOH·H₂O (140.18 mg, 3.34 mmol). The mixture was stirred at 30° C. for 16 h. The mixture was adjusted to pH 3 using IN HCl. The mixture was extracted with ethyl acetate (30 mL×2). The combined organic layers were washed with brine (30 mL×2), dried over Na₂SO₄, filtered and concentrated under reduced pressure to afford 2-(4-((3-(2,6-bis(benzyloxy)pyridin-3-yl)-1-methyl-1H-indazol-6-yl)amino)-3-methylphenyl)acetic acid (4, 390 mg, 667.05 μmol, 99% yield) as a yellow solid. The material was used in the next step without further purification. LCMS (ESI): m/z 585.1 [M+H]⁺

### 2-[[4-[1-(2,6-dioxo-3-piperidyl)-3-methyl-2-oxo-benzimidazol-5-yl]-1-piperidyl] methyl] cyclopropane carboxylic acid (3)

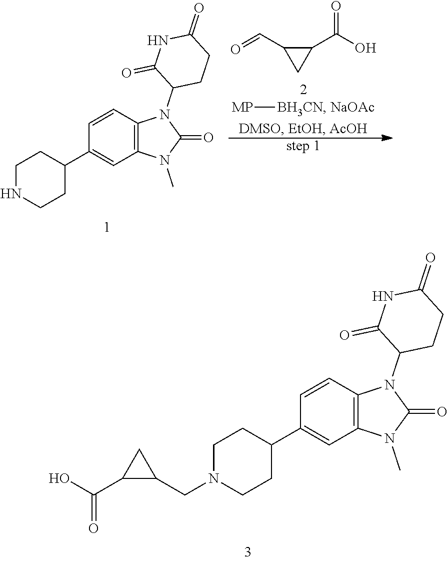

### Step 1: 2-[[4-[1-(2,6-dioxo-3-piperidyl)-3-methyl-2-oxo-benzimidazol-5-yl]-1-piperidyl] methyl] cyclopropane carboxylic acid (3)

Into a 50 mL single neck round bottom flask containing a well-stirred solution of 3-[3-methyl-2-oxo-5-(4-piperidyl)benzimidazol-1-yl]piperidine-2,6-dione (1, 700 mg, 1.53 mmol) and 2-formylcyclopropanecarboxylic acid (2, 174.99 mg, 1.53 mmol) in DMSO (2.5 mL) and ethanol (2.5 mL) was added sodium acetate (377.43 mg, 4.60 mmol) and acetic acid (920.98 mg, 15.34 mmol, 877.13 μL). After 30 min, MP-cyanoborohydride (2.04 mmol/g loading) (1.50 g, 3.07 mmol) was added. After 16 h, the reaction mixture was filtered, concentrated and purified by reverse phase column chromatography (120 g of C18 column; 0.1% TFA in water and acetonitrile) to afford 2-[[4-[1-(2,6-dioxo-3-piperidyl)-3-methyl-2-oxo-benzimidazol-5-yl]-1-piperidyl] methyl] cyclopropane carboxylic acid (3, 840 mg, 1.14 mmol, 74% yield) as an off-white solid. LCMS (ES+): m/z 441.2 [M+H]⁺

### 2-[4-[1-(2,6-dioxo-3-piperidyl)-3-methyl-2-oxo-benzimidazol-5-yl]pyrazol-1-yl]-N-[5-fluoro-7-hydroxy-6-(1,1,4-trioxo-1,2,5-thiadiazolidin-2-yl)-2-naphthyl]acetamide (Example 172)

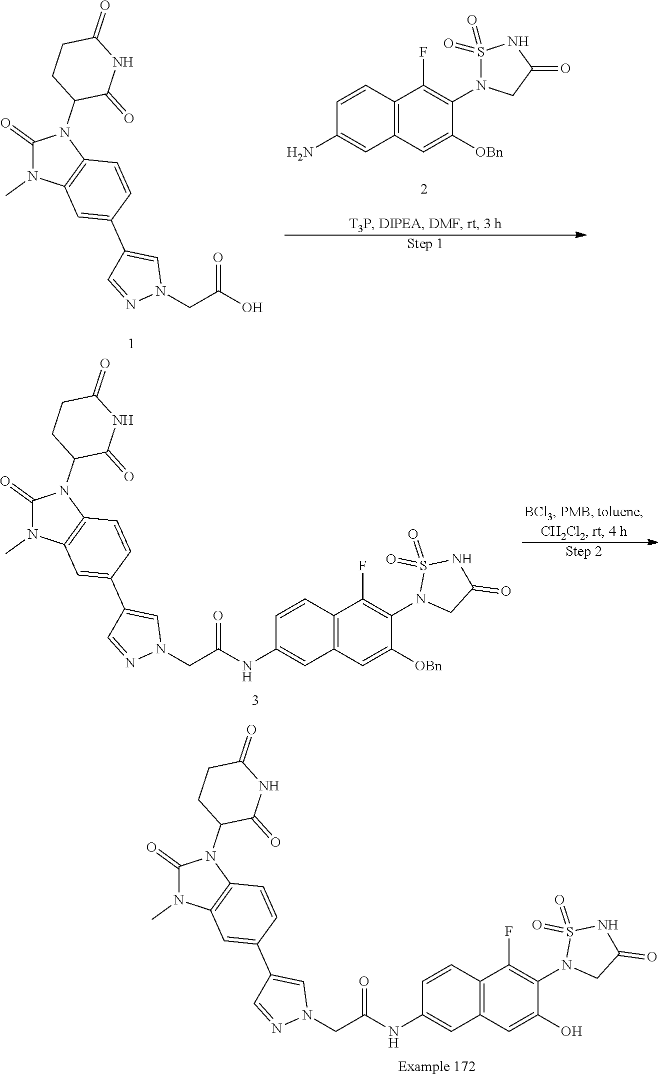 [SMILES: Cn1c(=O)n(C2CCC(=O)NC2=O)c2ccc(-c3cnn(CC(=O)O)c3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3cnn(CC(=O)Nc4ccc5c(F)c(N6CC(=O)NS6(=O)=O)c(OCc6ccccc6)cc5c4)c3)cc21 ; Cn1c(=O)n(C2CCC(=O)NC2=O)c2ccc(-c3cnn(CC(=O)Nc4ccc5c(F)c(N6CC(=O)NS6(=O)=O)c(O)cc5c4)c3)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-1H-pyrazol-1-yl)acetamide (3)

To a stirred solution of 2-[4-[1-(2,6-dioxo-3-piperidyl)-3-methyl-2-oxo-benzimidazol-5-yl]pyrazol-1-yl]acetic acid (1, 350 mg, 822.69 μmol) in DMF (3.5 mL) was added 5-(6-amino-3-benzyloxy-1-fluoro-2-naphthyl)-1,1-dioxo-1,2,5-thiadiazolidin-3-one (2, 448.25 mg, 822.69 μmol, TFA salt) and DIPEA (318.98 mg, 2.47 mmol, 429.89 μL) at (° C.) Then, 1-propanephosphonic anhydride (50% in ethyl acetate) (785.29 mg, 1.23 mmol) was added to the reaction mixture. After 2 h, the solvent was removed and the residue was suspended in water (2.5 mL). The solid was filtered and dried to obtain N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-1H-pyrazol-1-yl)acetamide (3, 560 mg, 499.98 μmol, 61% yield) as a brown solid. The material was used in the next step without further purification. LCMS (ES−): m/z 766.0 [M−H]⁻

### Step 2: 2-[4-[1-(2,6-dioxo-3-piperidyl)-3-methyl-2-oxo-benzimidazol-5-yl]pyrazol-1-yl]-N-[5-fluoro-7-hydroxy-6-(1,1,4-trioxo-1,2,5-thiadiazolidin-2-yl)-2-naphthyl]acetamide (Example 172)

To a stirred solution of N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)-1H-pyrazol-1-yl)acetamide (3, 300 mg, 266.06 μmol) in toluene (2 mL) and CH₂Cl₂ (2 mL) were added pentamethylbenzene (197.21 mg, 1.33 mmol, 215.06 μL) at −78° C. followed by boron trichloride (1.0 M in DCM) (623.48 mg, 5.32 mmol, 5.32 mL). After 5 h at RT, the reaction mixture was quenched by adding 5% MeOH in CH₂Cl₂ (10 mL). The solvent was removed and the residue was triturated with MTBE (2×50 mL), filtered and dried. The material was purified by reverse phase preparatory HPLC [Purification method: Column: Xbridge C-18: (20×150) mm; Mobile phase: A: 0.1% Formic acid in water, B: Acetonitrile] to afford 2-[4-[1-(2,6-dioxo-3-piperidyl)-3-methyl-2-oxo-benzimidazol-5-yl]pyrazol-1-yl]-N-[5-fluoro-7-hydroxy-6-(1,1,4-trioxo-1,2,5-thiadiazolidin-2-yl)-2-naphthyl]acetamide (Example 172, 80 mg, 105.28 μmol, 40% yield, formic acid salt). LCMS (ES+): m/z 678.5 [M+H]⁺

¹HNMR (400 MHZ, DMSO-d6): δ 11.13 (s, 1H), 10.65 (s, 1H), 10.50 (bs, 1H), 8.23 (s, 1H), 8.20 (s, 1H), 7.96 (s, 1H), 7.91 (d, J=8.80 Hz, 1H), 7.48-7.45 (m, 2H), 7.32-7.29 (m, 1H), 7.12 (d, J=8.40 Hz, 1H), 6.98 (s, 1H), 5.40-5.36 (m, 1H), 5.11 (s, 2H), 4.42 (s, 2H), 3.39 (s, 3H), 2.95-2.87 (m, 1H), 2.78-2.62 (m, 2H), 2.10-2.03 (m, 1H).

### N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)-1H-pyrazol-1-yl)propanamide (Example 173)

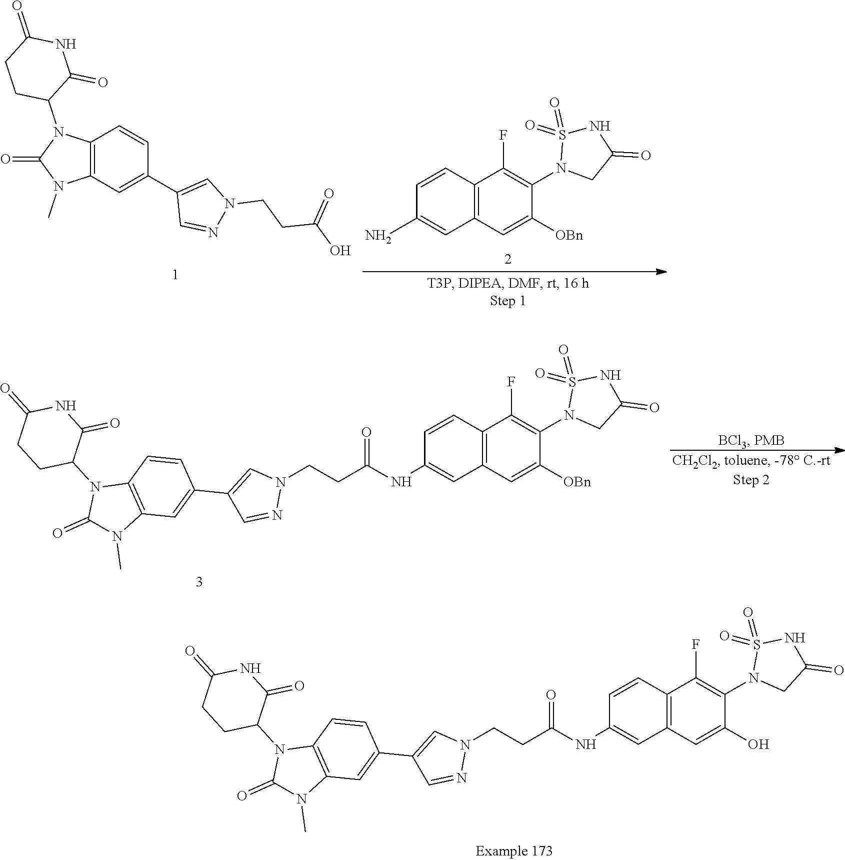 [SMILES: Cn1c(=O)n(C2CCC(=O)NC2=O)c2ccc(-c3cnn(CCC(=O)O)c3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(-c3cnn(CCC(=O)Nc4ccc5c(F)c(N6CC(=O)NS6(=O)=O)c(OCc6ccccc6)cc5c4)c3)cc21 ; Cn1c(=O)n(C2CCC(=O)NC2=O)c2ccc(-c3cnn(CCC(=O)Nc4ccc5c(F)c(N6CC(=O)NS6(=O)=O)c(O)cc5c4)c3)cc21]

### Step 1: N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)-1H-pyrazol-1-yl)propanamide (3)

Into a 20 mL vial containing a well-stirred solution of 5-(6-amino-3-benzyloxy-1-fluoro-2-naphthyl)-1,1-dioxo-1,2,5-thiadiazolidin-3-one (2, 230 mg, 439.04 μmol, TFA salt) in dry DMF (5 mL) were added DIPEA (283.72 mg, 2.20 mmol, 382.37 μL) and 3-[4-[1-(2,6-dioxo-3-piperidyl)-3-methyl-2-oxo-benzimidazol-5-yl] pyrazol-1-yl] propionic acid (1, 300 mg, 509.54 μmol, TFA salt) at 0° C. Then, propylphosphonic anhydride solution (≥50 wt. % in EtOAc) (838.17 mg, 1.32 mmol) was added at 0° C. After 16 h at RT, the reaction mixture was concentrated under reduced pressure and poured into ice cold water (50 mL) to obtain a solid that was filtered and dried to afford N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)-1H-pyrazol-1-yl)propanamide (3, 185 mg, 137.81 μmol, 31% yield) as a brown solid. LCMS (ES−): m/z 779.0 [M−H]⁻

### Step 2: N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)-1H-pyrazol-1-yl)propanamide (Example 173)

Into a 25 mL single neck round bottom flask containing a well-stirred solution of N-(7-(benzyloxy)-6-(1,1-dioxido-4-oxo-1,2,5-thiadiazolidin-2-yl)-5-fluoronaphthalen-2-yl)-3-(4-(1-(2,6-dioxopiperidin-3-yl)-3-methyl-2-oxo-2,3-dihydro-1H-benzo[d]imidazol-5-yl)-1H-pyrazol-1-yl)propanamide (3, 230 mg, 171.33 μmol) in dry DCM (3 mL) and toluene (3 mL) under nitrogen atmosphere was added pentamethylbenzene (253.99 mg, 1.71 mmol). The mixture was cooled to −78° C. and treated with BCl₃ (1.0 M solution in DCM) (3.43 mmol, 3.43 mL) via dropwise addition. After 2 h at RT, the reaction mixture was concentrated under reduced pressure and triturated with MTBE (50) mL) and purified by reverse-phase preparative-HPLC [Column: X-select C18 (150×19) mm, 5 μm; Mobile phase A: 0.1% TFA in water and Mobile Phase B: CH₃CN) to afford N-(6-(1,1-dioxido-4-oxo-1,2,5-thiadiazolidin-2-yl)-5-fluoro-7-hydroxynaphthalen-2-yl)-3-(4-(1-(2,6-dioxopiperidin-3-yl)-3-methyl-2-oxo-2,3-dihydro-1H-benzo[d]imidazol-5-yl)-1H-pyrazol-1-yl)propanamide (Example 173, 40 mg, 46.09 μmol, 27% yield, TFA salt) as an off-white solid. LCMS (ES+): m/z 691.0 [M+H]⁺

¹H-NMR (400 MHZ, DMSO-d6): δ 11.11 (s, 1H), 10.35 (brs, 1H), 10.31 (s, 1H), 8.17 (s, 2H), 7.91 (s, 1H), 7.85 (d, J=8.80 Hz, 1H), 7.42-7.40 (m, 2H), 7.25 (d, J=8.00 Hz, 1H), 7.09 (d, J=8.00 Hz, 1H), 6.96 (s, 1H), 5.36 (dd, J=5.60, 12.60 Hz, 1H), 4.46 (s, 2H), 4.36 (s, 2H), 3.32 (s, 3H), 3.01-2.86 (m, 3H), 3.00-2.54 (m, 2H), 2.03-2.01 (m, 1H).

### N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)acetamide (Example 174)

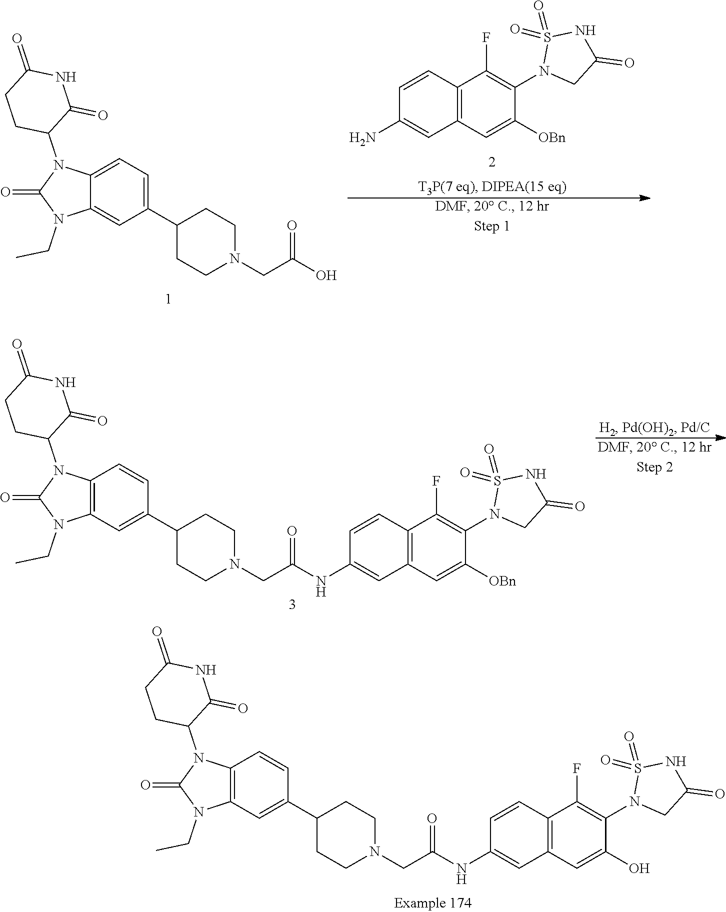 [SMILES: CCn1c(=O)n(C2CCC(=O)NC2=O)c2ccc(C3CCN(CC(=O)O)CC3)cc21 ; Nc1ccc2c(F)c(N3CC(=O)NS3(=O)=O)c(OCc3ccccc3)cc2c1 ; CCn1c(=O)n(C2CCC(=O)NC2=O)c2ccc(C3CCN(CC(=O)Nc4ccc5c(F)c(N6CC(=O)NS6(=O)=O)c(OCc6ccccc6)cc5c4)CC3)cc21 ; CCn1c(=O)n(C2CCC(=O)NC2=O)c2ccc(C3CCN(CC(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)acetamide (3)

To a solution of 2-(4-(1-(2,6-dioxopiperidin-3-yl)-3-ethyl-2-oxo-2,3-dihydro-1H-benzo[d]imidazol-5-yl)piperidin-1-yl)acetic acid (1, 390 mg, 941.00 μmol) in DMF (5 mL) were added 5-(6-amino-3-benzyloxy-1-fluoro-2-naphthyl)-1,1-dioxo-1,2,5-thiadiazolidin-3-one (2, 377.73 mg, 941.00 μmol), DIPEA (1.43 g, 14.11 mmol, 1.97 mL) and T₃P (50% in ethyl acetate) (4.19 g, 6.59 mmol). After 12 h, the reaction mixture was concentrated under reduced pressure. The residue was purified by reversed phase flash (flow: 30 mL/min; gradient: from 10-40% MeCN in water (0.1% TFA) over 15 min; column: Welch Ultimate XB-C18, 20-40 μm, 100 Å, I.D. 95 mm×H 365 mm) to afford N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)acetamide (3, 210 mg, 263.21 μmol, 28% yield) as a white solid.

### Step 2: N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)acetamide (Example 174)

To a solution of N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)acetamide (3, 100 mg, 125.34 μmol) in DMF (2 mL) was added Pd/C (20 mg, 10% purity) and Pd(OH)₂/C (20 mg, 10% purity). The suspension was degassed and purged with H₂ 3 times. The mixture was stirred under H₂ (15 Psi) for 12 h. The mixture was filtered and concentrated under reduced pressure. The residue was purified by prep-HPLC (flow: 25 mL/min; gradient: from 14-44% MeCN in water (0.1% TFA); column: Phenomenex Synergi C18 150×25 mm×10 μm) to afford N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-ethyl-2-oxo-2,3-dihydro-1H-benzo[d]imidazol-5-yl)piperidin-1-yl)acetamide (Example 174, 56 mg, 75.17 μmol, 60% yield, TFA salt) as a white solid. LCMS (ESI): m/z 708.6 [M+H]⁺

¹H NMR (400 MHZ, DMSO-d₆) δ: 11.11 (s, 1H), 10.80 (s, 1H), 10.00 (s, 1H), 9.79 (dt, J=3.2, 7.2 Hz, 1H), 8.12 (s, 1H), 7.92 (d, J=8.8 Hz, 1H), 7.49 (dd, J=1.6, 9.6 Hz, 1H), 7.14-7.08 (m, 2H), 7.02 (s, 1H), 6.94 (d, J=8.4 Hz, 1H), 5.36 (dd, J=5.6, 12.0 Hz, 1H), 4.25 (s, 2H), 4.17 (s, 2H), 3.93-3.88 (m, 2H), 3.69 (d, J=9.6 Hz, 2H), 3.31-3.26 (m, 2H), 2.94-2.87 (m, 2H), 2.72-2.65 (m, 2H), 2.13-1.99 (m, 5H), 1.25 (t, J=7.2 Hz, 3H).

### N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)pyridin-2-yl)acetamide (Example 175)

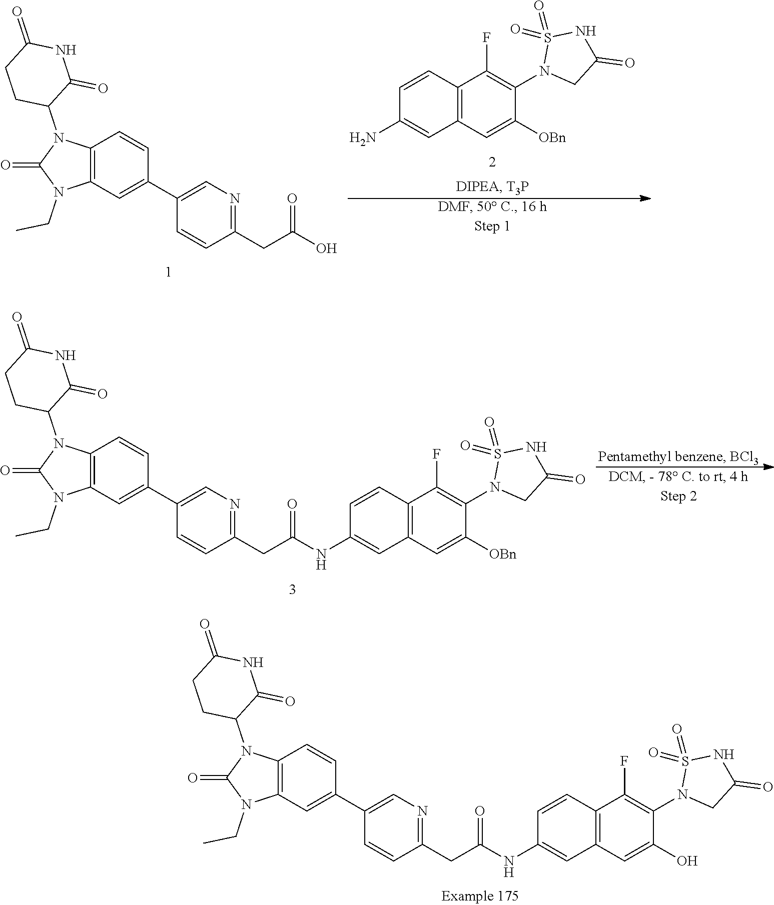 [SMILES: CCn1c(=O)n(C2CCC(=O)NC2=O)c2ccc(-c3ccc(CC(=O)O)nc3)cc21 ; Nc1ccc2c(F)c(N3CC(=O)NS3(=O)=O)c(OCc3ccccc3)cc2c1 ; CCn1c(=O)n(C2CCC(=O)NC2=O)c2ccc(-c3ccc(CC(=O)Nc4ccc5c(F)c(N6CC(=O)NS6(=O)=O)c(OCc6ccccc6)cc5c4)nc3)cc21 ; CCn1c(=O)n(C2CCC(=O)NC2=O)c2ccc(-c3ccc(CC(=O)Nc4ccc5c(F)c(N6CC(=O)NS6(=O)=O)c(O)cc5c4)nc3)cc21]

### Step 1: N-(7-(benzyloxy)-6-(1,1-dioxido-4-oxo-1,2,5-thiadiazolidin-2-yl)-5-fluoronaphthalen-2-yl)-2-(5-(1-(2,6-dioxopiperidin-3-yl)-3-methyl-2-oxo-2,3-dihydro-1H-benzo[d]imidazol-5-yl)pyridin-2-yl)acetamide (3)

To a solution of 2-(5-(1-(2,6-dioxopiperidin-3-yl)-3-methyl-2-oxo-2,3-dihydro-1H-benzo[d]imidazol-5-yl)pyridin-2-yl)acetic acid (1, 330 mg, 765.94 μmol, HCl salt) in DMF (3 mL) were added 5-(6-amino-3-(benzyloxy)-1-fluoronaphthalen-2-yl)-1,2,5-thiadiazolidin-3-one 1,1-dioxide (2, 394.79 mg, 765.94 μmol, TFA salt), DIPEA (1.48 g, 11.49 mmol, 2.00 mL,) and T₃P (50% purity in EtOAc) (3.41 g, 5.36 mmol). The mixture was stirred at 50° C. for 16 h. The mixture was filtered, concentrated and the residue purified by reversed phase flash (flow: 35 mL/min; gradient: from 50-100% MeCN in water (0.1% formic acid) over 30 min; column: Welch Ultimate XB_C18 20-40 μm: 120 Å) followed by prep-HPLC (flow: 25 mL/min; gradient: from 12-42% MeCN in water (10 mM NH₄HCO₃) over 10 min; column: Waters Xbridge 150×25 mm×5 μm) to afford N-(7-(benzyloxy)-6-(1,1-dioxido-4-oxo-1,2,5-thiadiazolidin-2-yl)-5-fluoronaphthalen-2-yl)-2-(5-(1-(2,6-dioxopiperidin-3-yl)-3-methyl-2-oxo-2,3-dihydro-1H-benzo[d]imidazol-5-yl)pyridin-2-yl)acetamide (3, 124 mg, 132.80 μmol, 17% yield, 84% purity) as a yellow solid. LCMS (ESI): m/z 778.3 [M+H]⁺

### Step 2: N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)pyridin-2-yl)acetamide (Example 175)

To a solution of N-(7-(benzyloxy)-6-(1,1-dioxido-4-oxo-1,2,5-thiadiazolidin-2-yl)-5-fluoronaphthalen-2-yl)-2-(5-(1-(2,6-dioxopiperidin-3-yl)-3-methyl-2-oxo-2,3-dihydro-1H-benzo[d]imidazol-5-yl)pyridin-2-yl)acetamide (3, 40 mg, 45.77 μmol) and 1,2,3,4,5-pentamethylbenzene (20.36 mg, 137.31 μmol, 22.20 μL) in DCM (2 mL) was added BCl₃ (1.0 M in DCM) (1.37 mL) at −78° C. under N₂ atmosphere. The mixture was stirred at 20° C. for 4 h. A solution of DCM:MeOH=10:1 (5 mL) was added at −78° C. and the mixture was concentrated and purified by prep-HPLC (flow: 30 mL/min; gradient: from 4-34% MeCN in water over 10 min; column: Phenomenex Gemini-NX C18 75×30 mm×3 μm) to afford N-(6-(1,1-dioxido-4-oxo-1,2,5-thiadiazolidin-2-yl)-5-fluoro-7-hydroxynaphthalen-2-yl)-2-(5-(1-(2,6-dioxopiperidin-3-yl)-3-methyl-2-oxo-2,3-dihydro-1H-benzo[d]imidazol-5-yl)pyridin-2-yl)acetamide (Example 175, 17.28 mg, 24.88 μmol, 54% yield) as a white solid. LCMS (ESI): m/z 688.0 [M+H]⁺

¹H NMR (400 MHZ, DMSO-d₆) δ 11.13 (s, 1H), 10.50 (s, 1H), 9.72 (s, 1H), 8.88 (d, J=2.0 HZ, 1H), 8.17 (s, 1H), 8.10 (dd, J=2.4, 8.0 Hz, 1H), 7.84 (d, J=8.8 Hz, 1H), 7.61 (d, J=1.6 Hz, 1H), 7.53 (d, J=8.0 Hz, 1H), 7.47 (dd, J=1.6, 9.2 Hz, 1H), 7.43 (dd, J=1.6, 8.4 Hz, 1H), 7.25 (d, J=8.0 Hz, 1H), 6.94 (s, 1H), 5.42 (dd, J=5.2, 12.8 Hz, 1H), 4.07 (s, 2H), 3.96 (s, 2H), 3.42 (s, 3H), 2.96-2.87 (m, 1H), 2.81-2.73 (m, 1H), 2.69-2.65 (m, 1H), 2.11-2.02 (m, 1H).

### N-(6-(1,1-dioxido-4-oxo-1,2,5-thiadiazolidin-2-yl)-5-fluoro-7-hydroxynaphthalen-2-yl)-2-((4-(1-(2,6-dioxopiperidin-3-yl)-3-methyl-2-oxo-2,3-dihydro-1H-benzo[d]imidazol-5-yl) piperidin-1-yl) methyl)cyclopropane-1-carboxamide (Example 176)

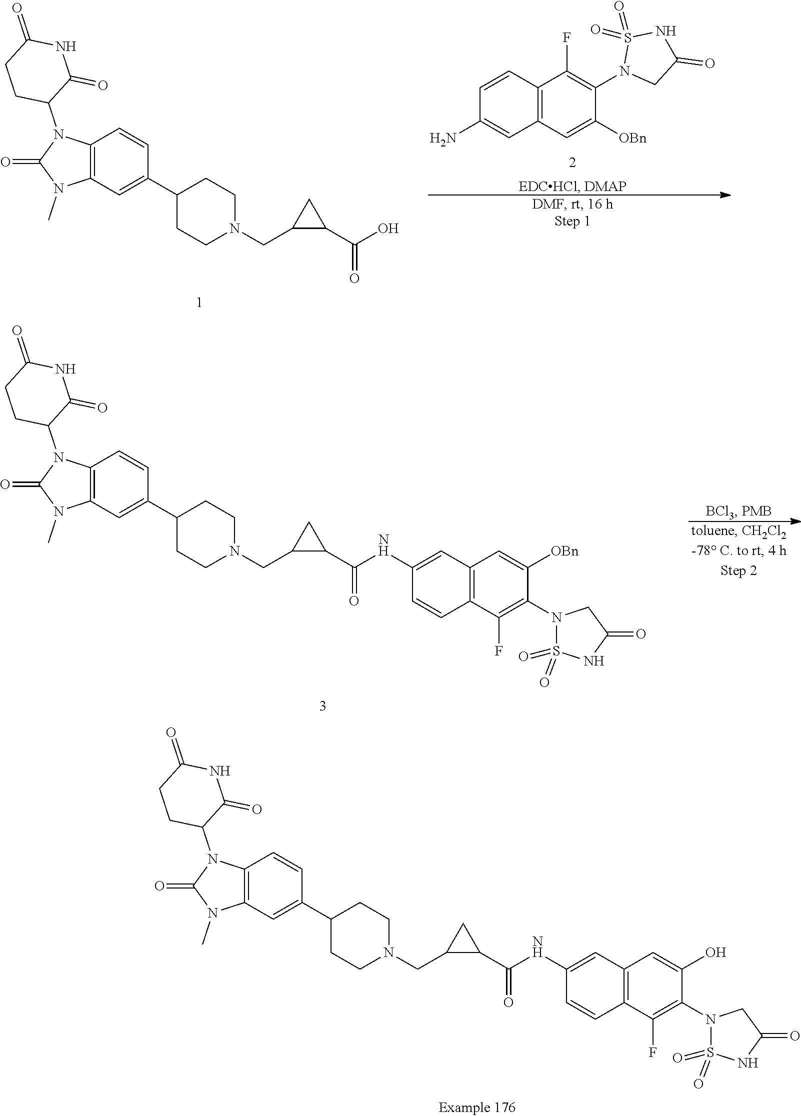 [SMILES: Cn1c(=O)n(C2CCC(=O)NC2=O)c2ccc(C3CCN(CC4CC4C(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(CC4CC4C(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN(CC4CC4C(=O)Nc4ccc5c(F)c(N6CC(=O)NS6(=O)=O)c(O)cc5c4)CC3)cc21]

### Step 1: N-(7-(benzyloxy)-6-(1,1-dioxido-4-oxo-1,2,5-thiadiazolidin-2-yl)-5-fluoronaphthalen-2-yl)-2-((4-(1-(2,6-dioxopiperidin-3-yl)-3-methyl-2-oxo-2,3-dihydro-1H-benzo[d]imidazol-5-yl) piperidin-1-yl) methyl) cyclopropane-1-carboxamide (3)

Into a 50 mL single neck round bottom flask containing a well-stirred solution of 2-[[4-[1-(2,6-dioxo-3-piperidyl)-3-methyl-2-oxo-benzimidazol-5-yl]-1-piperidyl] methyl] cyclopropane carboxylic acid (1, 300 mg, 510.79 μmol) in DMF (5 mL) was added EDC HCl (293.76 mg, 1.53 mmol) and DMAP (312.01 mg, 2.55 mmol). After 1 h, 5-(6-amino-3-benzyloxy-1-fluoro-2-naphthyl)-1,1-dioxo-1,2,5-thiadiazolidin-3-one (2, 263.28 mg, 510.79 μmol, TFA salt) was added. After 16 h, the reaction mixture was concentrated and diluted with ice cold water. The solid was filtered and dried to afford N-(7-(benzyloxy)-6-(1,1-dioxido-4-oxo-1,2,5-thiadiazolidin-2-yl)-5-fluoronaphthalen-2-yl)-2-((4-(1-(2,6-dioxopiperidin-3-yl)-3-methyl-2-oxo-2,3-dihydro-1H-benzo[d]imidazol-5-yl) piperidin-1-yl) methyl) cyclopropane-1-carboxamide (3, 300 mg, 282.67 μmol, 55% yield) as an off white solid. The material was used in the next step without further purification. LCMS (ES+): m/z 824.2 [M+H]⁺

### Step 2: N-(6-(1,1-dioxido-4-oxo-1,2,5-thiadiazolidin-2-yl)-5-fluoro-7-hydroxynaphthalen-2-yl)-2-((4-(1-(2,6-dioxopiperidin-3-yl)-3-methyl-2-oxo-2,3-dihydro-1H-benzo[d]imidazol-5-yl) piperidin-1-yl) methyl)cyclopropane-1-carboxamide (Example 176)

Into a 50 mL single neck round bottom flask containing a well-stirred solution of N-(7-(benzyloxy)-6-(1,1-dioxido-4-oxo-1,2,5-thiadiazolidin-2-yl)-5-fluoronaphthalen-2-yl)-2-((4-(1-(2,6-dioxopiperidin-3-yl)-3-methyl-2-oxo-2,3-dihydro-1H-benzo[d]imidazol-5-yl) piperidin-1-yl) methyl) cyclopropane-1-carboxamide (3, 250 mg, 235.56 μmol) in DCM (10 mL) and toluene (10 mL) was added pentamethylbenzene (174.60 mg, 1.18 mmol, 0.2 mL) under nitrogen atmosphere. The reaction mixture was then cooled to −78° C. and BCl₃ (1.0 M in DCM) (4.71 mmol, 4.7 mL) was added dropwise. After 4 h at RT, the reaction mixture was cooled to −78° C. and quenched with 5% MeOH in DCM (1 mL). The reaction mixture was concentrated under reduced pressure and purified by reverse-phase preparative HPLC [Column: XBRIDGE C18 (30×150) mm, 5 μm; Mobile Phase A: 0.1% Formic acid in Water and Mobile Phase B: CH₃CN] to afford N-(6-(1,1-dioxido-4-oxo-1,2,5-thiadiazolidin-2-yl)-5-fluoro-7-hydroxynaphthalen-2-yl)-2-((4-(1-(2,6-dioxopiperidin-3-yl)-3-methyl-2-oxo-2,3-dihydro-1H-benzo[d]imidazol-5-yl) piperidin-1-yl) methyl)cyclopropane-1-carboxamide (Example 176, 78 mg, 94.68 μmol, 40% yield, formic acid salt) as an off-white solid. LCMS (ES+): m/z 734.3 [M+H]⁺

¹H-NMR (400 MHZ, DMSO-d₆): δ 11.11 (s, 1H), 10.53 (s, 1H), 9.75 (s, 1H), 9.25 (brs, 1H), 8.14 (s, 1H), 7.85-7.83 (m, 1H), 7.46 (d, J=8.40 Hz, 1H), 7.08-7.06 (m, 2H), 6.94 (s, 2H), 5.38-5.35 (m, 1H), 4.08 (s, 2H), 3.72-3.60 (m, 2H), 3.34 (s, 3H), 3.21-3.15 (m, 2H), 2.95-2.61 (m, 4H), 2.15-1.99 (m, 7H), 1.75-1.60 (m, 1H), 1.30-1.20 (m, 1H), 1.12-1.00 (m, 1H).

### N-(6-(1,1-dioxido-4-oxo-1,2,5-thiadiazolidin-2-yl)-5-fluoro-7-hydroxynaphthalen-2-yl)-2-(4-(3-(2,6-dioxopiperidin-3-yl)-1-methyl-1H-indazol-6-yl)phenyl)acetamide (Example 177)

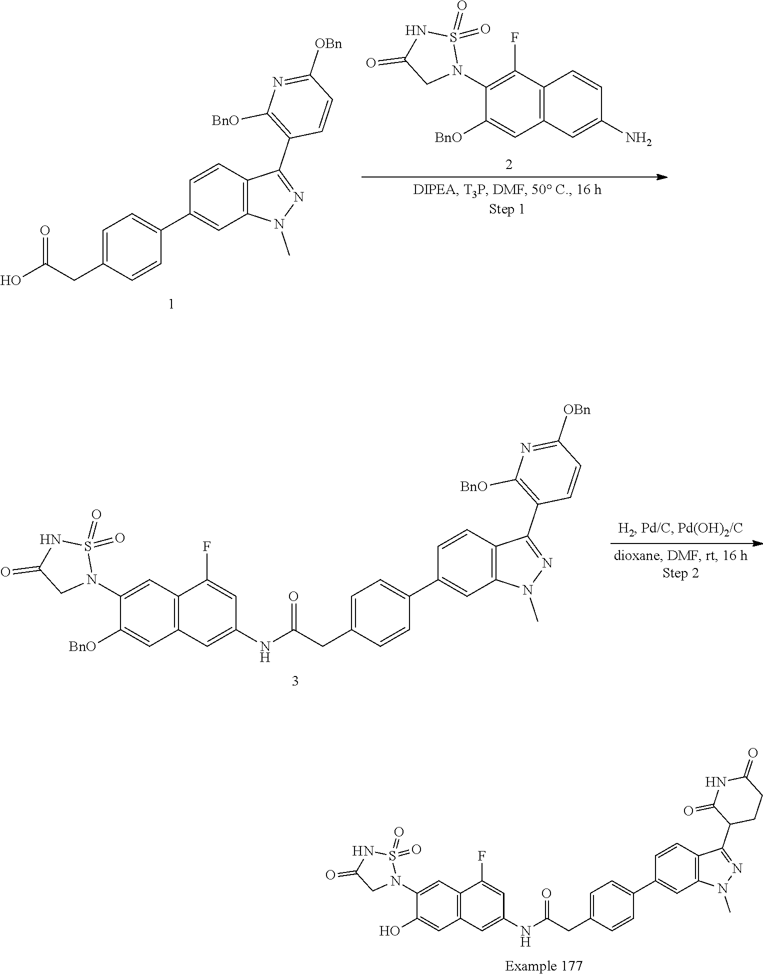 [SMILES: Cn1nc(-c2ccc(OCc3ccccc3)nc2OCc2ccccc2)c2ccc(-c3ccc(CC(=O)O)cc3)cc21 ; Nc1ccc2c(F)c(N3CC(=O)NS3(=O)=O)c(OCc3ccccc3)cc2c1 ; Cn1nc(-c2ccc(OCc3ccccc3)nc2OCc2ccccc2)c2ccc(-c3ccc(CC(=O)Nc4cc(F)c5cc(N6CC(=O)NS6(=O)=O)c(OCc6ccccc6)cc5c4)cc3)cc21 ; Cn1nc(C2CCC(=O)NC2=O)c2ccc(-c3ccc(CC(=O)Nc4cc(F)c5cc(N6CC(=O)NS6(=O)=O)c(O)cc5c4)cc3)cc21]

### Step 1: N-(7-(benzyloxy)-6-(1,1-dioxido-4-oxo-1,2,5-thiadiazolidin-2-yl)-5-fluoronaphthalen-2-yl)-2-(4-(3-(2,6-bis(benzyloxy)pyridin-3-yl)-1-methyl-1H-indazol-6-yl)phenyl)acetamide (3)

To a solution of 2-(4-(3-(2,6-bis(benzyloxy)pyridin-3-yl)-1-methyl-1H-indazol-6-yl)phenyl)acetic acid (1, 300 mg, 539.94 μmol), 5-(6-amino-3-benzyloxy-1-fluoro-2-naphthyl)-1,1-dioxo-1,2,5-thiadiazolidin-3-one (2, 278.30 mg, 539.94 μmol) in DMF (5 mL) were added DIPEA (1.05 g, 8.10 mmol, 1.41 mL) and T₃P (50% in EtOAc) (2.41 g, 3.78 mmol). The mixture was stirred at 50° C. for 16 h. The reaction was quenched by addition of H₂O (50 mL) at 0° C., and extracted with ethyl acetate (30 mL×3), dried over Na₂SO₄, filtered and concentrated under reduced pressure. The residue was purified by column chromatography (SiO₂, petroleum ether:ethyl acetate=100:1 to 1:1) to afford N-(7-(benzyloxy)-6-(1,1-dioxido-4-oxo-1,2,5-thiadiazolidin-2-yl)-5-fluoronaphthalen-2-yl)-2-(4-(3-(2,6-bis(benzyloxy)pyridin-3-yl)-1-methyl-1H-indazol-6-yl)phenyl)acetamide (3, 200 mg, 212.99 μmol, 39% yield) as a white solid. LCMS (ESI): m/z 940.1 [M+H]⁺

### Step 2: N-(6-(1,1-dioxido-4-oxo-1,2,5-thiadiazolidin-2-yl)-5-fluoro-7-hydroxynaphthalen-2-yl)-2-(4-(3-(2,6-dioxopiperidin-3-yl)-1-methyl-1H-indazol-6-yl)phenyl)acetamide (Example 177)

To a solution of N-(7-(benzyloxy)-6-(1,1-dioxido-4-oxo-1,2,5-thiadiazolidin-2-yl)-5-fluoronaphthalen-2-yl)-2-(4-(3-(2,6-bis(benzyloxy)pyridin-3-yl)-1-methyl-1H-indazol-6-yl)phenyl)acetamide (3, 70 mg, 74.55 μmol) in 1,4-dioxane (1 mL) and DMF (1 mL) was added Pd/C (50 mg, 10% purity) and Pd(OH)₂/C (50 mg, 10% purity). The mixture was stirred at 20° C. for 16 h under H₂ atmosphere (15 Psi). The reaction mixture was filtered and concentrated under reduced pressure. The residue was purified by prep-HPLC (flow: 25 mL/min; gradient: from 62-32% water (0.1% TFA)-ACN; column: Phenomenex Gemini-NX C18 75×30 mm×3 μm) to afford N-(6-(1,1-dioxido-4-oxo-1,2,5-thiadiazolidin-2-yl)-5-fluoro-7-hydroxynaphthalen-2-yl)-2-(4-(3-(2,6-dioxopiperidin-3-yl)-1-methyl-1H-indazol-6-yl)phenyl)acetamide (Example 177, 30 mg, 37.08 μmol, 50% yield, TFA salt) as an off-white solid. LCMS (ESI): m/z 671.2 [M+H]⁺

¹H NMR (400 MHZ, DMSO-d₆) δ 10.90 (s, 1H), 10.48-10.41 (m, 1H), 10.13-10.03 (m, 1H), 8.21-8.14 (m, 1H), 7.89-7.83 (m, 2H), 7.80-7.73 (m, 3H), 7.53-7.41 (m, 4H), 6.98-6.91 (m, 1H), 4.44-4.36 (m, 1H), 4.28-4.20 (m, 2H), 4.08-4.03 (m, 3H), 3.79-3.74 (m, 2H), 2.70-2.62 (m, 2H), 2.41-2.36 (m, 2H).

### N-(6-(1,1-dioxido-4-oxo-1,2,5-thiadiazolidin-2-yl)-5-fluoro-7-hydroxynaphthalen-2-yl)-2-((2R)-4-(1-(2,6-dioxopiperidin-3-yl)-3-methyl-2-oxo-2,3-dihydro-1H-benzo[d]imidazol-5-yl)-2-methylpiperidin-1-yl)acetamide (Example 178)

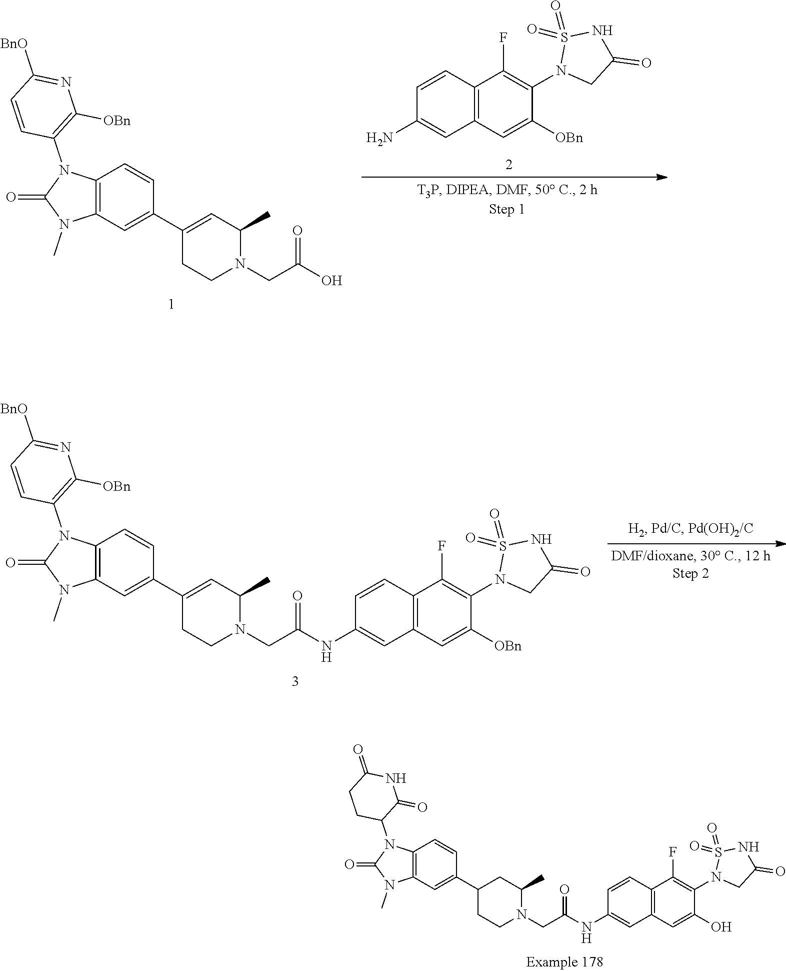 [SMILES: C[C@@H]1C=C(c2ccc3c(c2)n(C)c(=O)n3-c2ccc(OCc3ccccc3)nc2OCc2ccccc2)CCN1CC(=O)O ; Nc1ccc2c(F)c(N3CC(=O)NS3(=O)=O)c(OCc3ccccc3)cc2c1 ; C[C@@H]1C=C(c2ccc3c(c2)n(C)c(=O)n3-c2ccc(OCc3ccccc3)nc2OCc2ccccc2)CCN1CC(=O)Nc1ccc2c(F)c(N3CC(=O)NS3(=O)=O)c(OCc3ccccc3)cc2c1 ; C[C@@H]1CC(c2ccc3c(c2)n(C)c(=O)n3C2CCC(=O)NC2=O)CCN1CC(=O)Nc1ccc2c(F)c(N3CC(=O)NS3(=O)=O)c(O)cc2c1]

### Step 1: (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetamide (3)

To a solution of (R)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetic acid (1, 300 mg, 478.37 μmol) in DMF (3 mL) were added 5-(6-amino-3-(benzyloxy)-1-fluoronaphthalen-2-yl)-1,2,5-thiadiazolidin-3-one 1,1-dioxide (2, 246.57 mg, 478.37 μmol, TFA salt), DIPEA (927.39 mg, 7.18 mmol, 15 eq) and T₃P (50% purity in EtOAc) (2.13 g, 3.35 mmol). The mixture was stirred at 50° C. for 2 h. The mixture was filtered, concentrated and purified by prep-HPLC (flow: 60 mL/min; gradient: from 61-91% water (10 mM NH₄HCO₃) in MeCN over 10 min; column: Waters Xbridge 150×25 mm×5 μm) to afford (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetamide (3, 50 mg, 49.28 μmol, 10% yield) as a yellow solid. LC-MS (ES+): m/z 974.4 [M+H]⁺

### Step 2: N-(6-(1,1-dioxido-4-oxo-1,2,5-thiadiazolidin-2-yl)-5-fluoro-7-hydroxynaphthalen-2-yl)-2-((2R)-4-(1-(2,6-dioxopiperidin-3-yl)-3-methyl-2-oxo-2,3-dihydro-1H-benzo[d]imidazol-5-yl)-2-methylpiperidin-1-yl)acetamide (Example 178)

To a solution of (R)—N-(7-(benzyloxy)-6-(1,1-dioxido-4-oxo-1,2,5-thiadiazolidin-2-yl)-5-fluoronaphthalen-2-yl)-2-(4-(1-(2,6-bis(benzyloxy)pyridin-3-yl)-3-methyl-2-oxo-2,3-dihydro-1H-benzo[d]imidazol-5-yl)-2-methyl-5,6-dihydropyridin-1(2H)-yl)acetamide (3, 40 mg, 41.07 μmol) in dioxane (1 mL) and DMF (1 mL) were added Pd/C (20.00 mg, 10% purity) and Pd(OH)₂ (20.00 mg, 20% purity). The mixture was stirred at 30° C. under H₂ atmosphere (15 Psi) for 12 h. The mixture was filtered, concentrated and purified by prep-HPLC (flow: 28 mL/min; gradient: from 12-32% water (0.05% HCl) in MeCN over 6.5 min; column: 3_PhenomenexLuna C18 75×30 mm×3 μm)) to give N-(6-(1,1-dioxido-4-oxo-1,2,5-thiadiazolidin-2-yl)-5-fluoro-7-hydroxynaphthalen-2-yl)-2-((2R)-4-(1-(2,6-dioxopiperidin-3-yl)-3-methyl-2-oxo-2,3-dihydro-1H-benzo[d]imidazol-5-yl)-2-methylpiperidin-1-yl)acetamide (Example 178, 9.1 mg, 11.74 μmol, 29% yield, HCl salt) as a pink solid. LC-MS (ESI): m/z 708.5[M+H]⁺

¹H NMR (400 MHZ, DMSO-d₆) δ=11.15-10.99 (m, 2H), 10.54-10.30 (br s, 1H), 10.01 (br s, 1H), 8.16 (s, 1H), 7.93 (d, J=8.8 Hz, 1H), 7.54 (dd, J=1.6, 9.2 Hz, 1H), 7.14-7.02 (m, 3H), 6.94 (d, J=8.4 Hz, 1H), 5.36 (dd, J=5.6, 12.8 Hz, 1H), 4.49 (d, J=16.0 Hz, 1H), 4.36 (s, 2H), 4.12 (b dd, J=3.6, 16.8 Hz, 1H), 3.76-3.72 (m, 1H), 3.41-3.33 (m, 4H), 3.07-2.85 (m, 2H), 2.77-2.58 (m, 3H), 2.20-1.88 (m, 5H), 1.48-1.25 (m, 3H).

### N-(6-(1,1-dioxido-4-oxo-1,2,5-thiadiazolidin-2-yl)-5-fluoro-7-hydroxynaphthalen-2-yl)-2-((trans)-4-(3-(2,6-dioxopiperidin-3-yl)-1-methyl-1H-indazol-6-yl)cyclohexyl)acetamide (Example 179, from First Eluted Fraction)

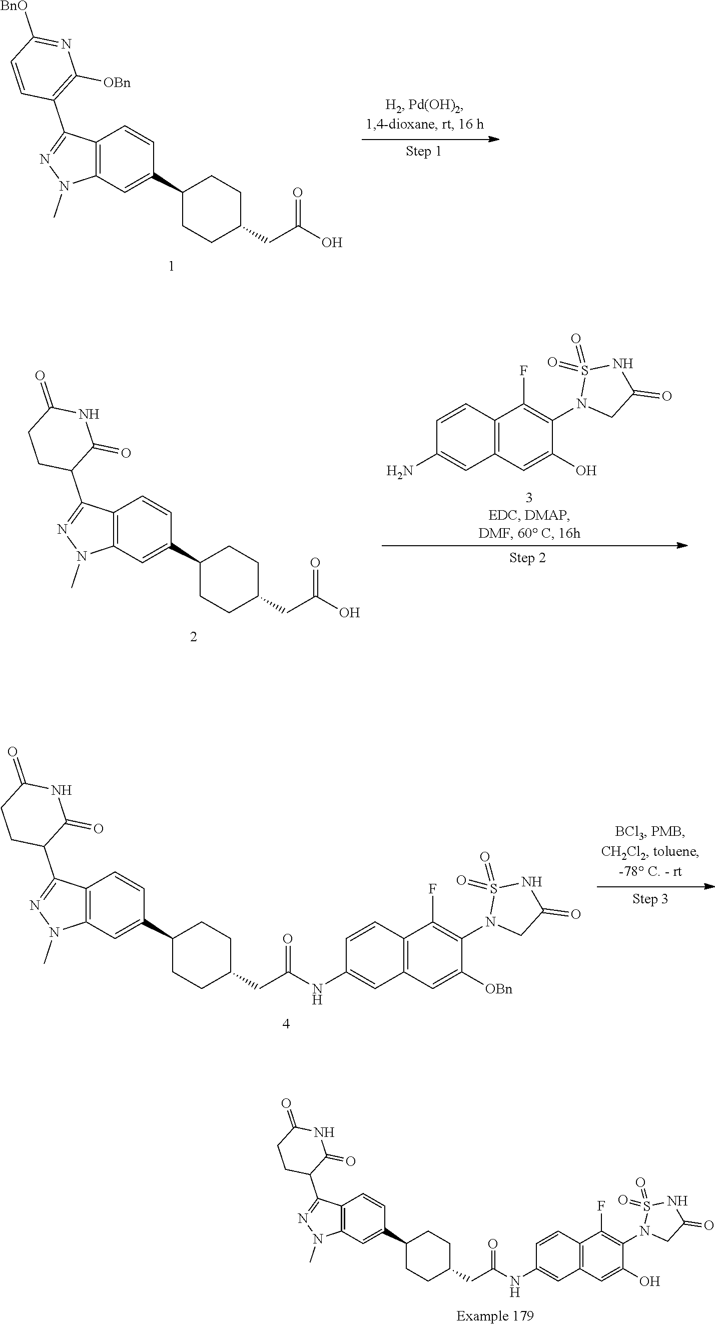 [SMILES: Cn1nc(-c2ccc(OCc3ccccc3)nc2OCc2ccccc2)c2ccc([C@H]3CC[C@H](CC(=O)O)CC3)cc21 ; Cn1nc(C2CCC(=O)NC2=O)c2ccc([C@H]3CC[C@H](CC(=O)O)CC3)cc21 ; Nc1ccc2c(F)c(N3CC(=O)NS3(=O)=O)c(O)cc2c1 ; Cn1nc(C2CCC(=O)NC2=O)c2ccc([C@H]3CC[C@H](CC(=O)Nc4ccc5c(F)c(N6CC(=O)NS6(=O)=O)c(OCc6ccccc6)cc5c4)CC3)cc21 ; Cn1nc(C2CCC(=O)NC2=O)c2ccc([C@H]3CC[C@H](CC(=O)Nc4ccc5c(F)c(N6CC(=O)NS6(=O)=O)c(O)cc5c4)CC3)cc21]

### Step 1: 2-((trans)-4-(3-(2,6-dioxopiperidin-3-yl)-1-methyl-1H-indazol-6-yl)cyclohexyl)acetic acid (2)

Into a 50 mL single neck round bottom flask containing a well-stirred solution of 2-((trans)-4-(3-(2,6-bis(benzyloxy)pyridin-3-yl)-1-methyl-1H-indazol-6-yl)cyclohexyl)acetic acid (1, 500 mg, 835.01 μmol) in anhydrous 1,4-dioxane (10 mL) was added palladium hydroxide on carbon (20 wt. % 50% water) (500 mg, 712.05 μmol, 20% purity). The suspension was stirred at RT under hydrogen atmosphere (bladder). After 16 h, the reaction mixture was filtered through Celite and washed with 1,4-dioxane (250 mL). The filtrate was concentrated under reduced pressure to afford 2-((trans)-4-(3-(2,6-dioxopiperidin-3-yl)-1-methyl-1H-indazol-6-yl)cyclohexyl)acetic acid (2, 320 mg, 808.68 μmol, 97% yield) as an off-white solid. The material was used in the next step without further purification. LCMS (ES+): m/z 384.2 [M+H]⁺

### Step 2: N-(7-(benzyloxy)-6-(1,1-dioxido-4-oxo-1,2,5-thiadiazolidin-2-yl)-5-fluoronaphthalen-2-yl)-2-((trans)-4-(3-(2,6-dioxopiperidin-3-yl)-1-methyl-1H-indazol-6-yl)cyclohexyl)acetamide (4)

Into a 20 mL vial containing a well-stirred solution of 2-((trans)-4-(3-(2,6-dioxopiperidin-3-yl)-1-methyl-1H-indazol-6-yl)cyclohexyl)acetic acid (2, 220.00 mg, 554.76 μmol) in anhydrous DMF (5 mL) were added N,N-dimethylpyridin-4-amine (338.88 mg, 2.77 mmol) and N-(3-dimethylaminopropyl)-N′-ethylcarbodiimide hydrochloride (319.04 mg, 1.66 mmol) followed by 5-(6-amino-3-benzyloxy-1-fluoro-2-naphthyl)-1,1-dioxo-1,2,5-thiadiazolidin-3-one (3, 307.47 mg, 554.76 μmol, TFA salt). After 16 h at 60° C. the volatiles were removed under reduced pressure, and the residue was quenched with 1.5 N HCl (5 mL). The solid was filtered and dried to afford N-(7-(benzyloxy)-6-(1,1-dioxido-4-oxo-1,2,5-thiadiazolidin-2-yl)-5-fluoronaphthalen-2-yl)-2-((trans)-4-(3-(2,6-dioxopiperidin-3-yl)-1-methyl-1H-indazol-6-yl)cyclohexyl)acetamide (4, 210 mg, 107.46 μmol, 19% yield, 39% purity) as an off-white solid. LCMS (ES−): m/z 765.2 [M−H]⁻

### Step 3: N-(6-(1,1-dioxido-4-oxo-1,2,5-thiadiazolidin-2-yl)-5-fluoro-7-hydroxynaphthalen-2-yl)-2-((trans)-4-(3-(2,6-dioxopiperidin-3-yl)-1-methyl-1H-indazol-6-yl)cyclohexyl)acetamide (Example 179)

Into a 50 mL single-neck round-bottomed flask containing a well-stirred solution of N-(7-(benzyloxy)-6-(1,1-dioxido-4-oxo-1,2,5-thiadiazolidin-2-yl)-5-fluoronaphthalen-2-yl)-2-((trans)-4-(3-(2,6-dioxopiperidin-3-yl)-1-methyl-1H-indazol-6-yl)cyclohexyl)acetamide (4, 210 mg, 273.85 μmol) and pentamethylbenzene (202.99 mg, 1.37 mmol, 221.36 μL) in DCM (2.5 mL) and toluene (2.5 mL) was added boron trichloride (1.0 M in DCM) (5.48 mmol, 5 mL) at −78° C. The reaction mixture was stirred at RT. Upon completion, the reaction mixture was cooled to −78° C. and quenched with 5% methanol in DCM (2 mL). The volatiles were evaporated under reduced pressure and the residue was triturated with MTBE (30 mL) and filtered. The crude compound was purified by reverse phase prep HPLC [Purification method: Column: X Bridge C 8 (150×19.1 mm) 5 μm; Mobile phase A: 10 mm Ammonium acetate in water; Mobile phase B: Acetonitrile] to afford N-(6-(1,1-dioxido-4-oxo-1,2,5-thiadiazolidin-2-yl)-5-fluoro-7-hydroxynaphthalen-2-yl)-2-((trans)-4-(3-(2,6-dioxopiperidin-3-yl)-1-methyl-1H-indazol-6-yl)cyclohexyl)acetamide (Example 179, 34 mg, 47.71 μmol, 17% yield) as an off white solid. LCMS (ES+): m/z 677.0 [M+H]⁺

¹HNMR (400 MHZ, DMSO-d6): δ 10.90 (s, 1H), 10.17 (s, 1H), 9.75 (s, 1H), 8.18 (s, 1H), 7.82 (d, J=8.80 Hz, 1H), 7.65-7.61 (m, 1H), 7.46-7.42 (m, 2H), 7.10-6.94 (m, 2H), 6.55 (s, 1H), 4.36-4.33 (m, 1H), 4.07 (s, 2H), 3.99 (s, 3H), 2.73-2.63 (m, 6H), 2.38-2.34 (m, 2H), 2.20-2.15 (m, 1H), 1.85-1.83 (m, 2H), 1.76-1.70 (m, 6H).

### N-(6-(1,1-dioxido-4-oxo-1,2,5-thiadiazolidin-2-yl)-5-fluoro-7-hydroxynaphthalen-2-yl)-2-((cis)-4-(3-(2,6-dioxopiperidin-3-yl)-1-methyl-1H-indazol-6-yl)cyclohexyl)acetamide (Example 180)

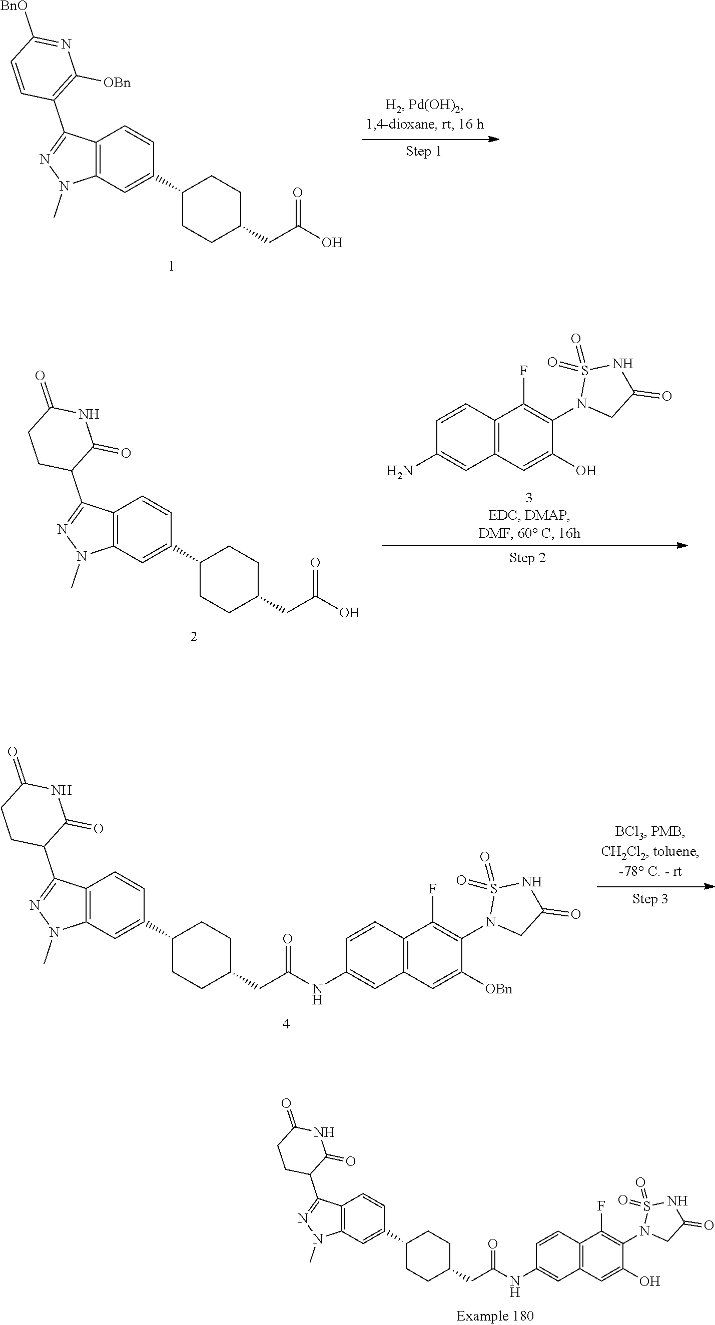 [SMILES: Cn1nc(-c2ccc(OCc3ccccc3)nc2OCc2ccccc2)c2ccc([C@H]3CC[C@@H](CC(=O)O)CC3)cc21 ; Cn1nc(C2CCC(=O)NC2=O)c2ccc([C@H]3CC[C@@H](CC(=O)O)CC3)cc21 ; Nc1ccc2c(F)c(N3CC(=O)NS3(=O)=O)c(O)cc2c1 ; Cn1nc(C2CCC(=O)NC2=O)c2ccc([C@H]3CC[C@@H](CC(=O)Nc4ccc5c(F)c(N6CC(=O)NS6(=O)=O)c(OCc6ccccc6)cc5c4)CC3)cc21 ; Cn1nc(C2CCC(=O)NC2=O)c2ccc([C@H]3CC[C@@H](CC(=O)Nc4ccc5c(F)c(N6CC(=O)NS6(=O)=O)c(O)cc5c4)CC3)cc21]

N-(6-(1,1-dioxido-4-oxo-1,2,5-thiadiazolidin-2-yl)-5-fluoro-7-hydroxynaphthalen-2-yl)-2-((cis)-4-(3-(2,6-dioxopiperidin-3-yl)-1-methyl-1H-indazol-6-yl)cyclohexyl)acetamide (Example 180, 35 mg, 50.43 μmol, 15% yield) was synthesized from 2-((cis)-4-(3-(2,6-bis(benzyloxy)pyridin-3-yl)-1-methyl-1H-indazol-6-yl)cyclohexyl)acetic acid (1) and 5-(6-amino-3-benzyloxy-1-fluoro-2-naphthyl)-1,1-dioxo-1,2,5-thiadiazolidin-3-one (3) over three steps following the same procedure as N-(6-(1,1-dioxido-4-oxo-1,2,5-thiadiazolidin-2-yl)-5-fluoro-7-hydroxynaphthalen-2-yl)-2-((trans)-4-(3-(2,6-dioxopiperidin-3-yl)-1-methyl-1H-indazol-6-yl)cyclohexyl)acetamide (Example 180)

LCMS (ES+): m/z 677.2 [M+H]⁺

¹HNMR (400 MHZ, DMSO-d6): δ 10.88 (s, 1H), 10.12 (s, 1H), 8.17 (s, 1H), 7.82 (d, J=8.80 Hz, 1H), 7.60 (d, J=8.40 Hz, 1H), 7.44-7.43 (m, 2H), 7.05 (d, J=8.40 Hz, 1H), 6.94 (d, J=Hz, 1H), 4.35-4.31 (m, 1H), 4.07 (s, 2H), 3.97 (s, 3H), 2.70-2.65 (m, 4H), 2.36-2.34 (m, 3H), 2.19-2.15 (m, 1H), 1.93-1.87 (m, 5H), 1.63-1.60 (m, 2H), 1.24-1.22 (m, 2H).

### 1-[2-[[1-[1-(2,6-dioxo-3-piperidyl)-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Example 181)

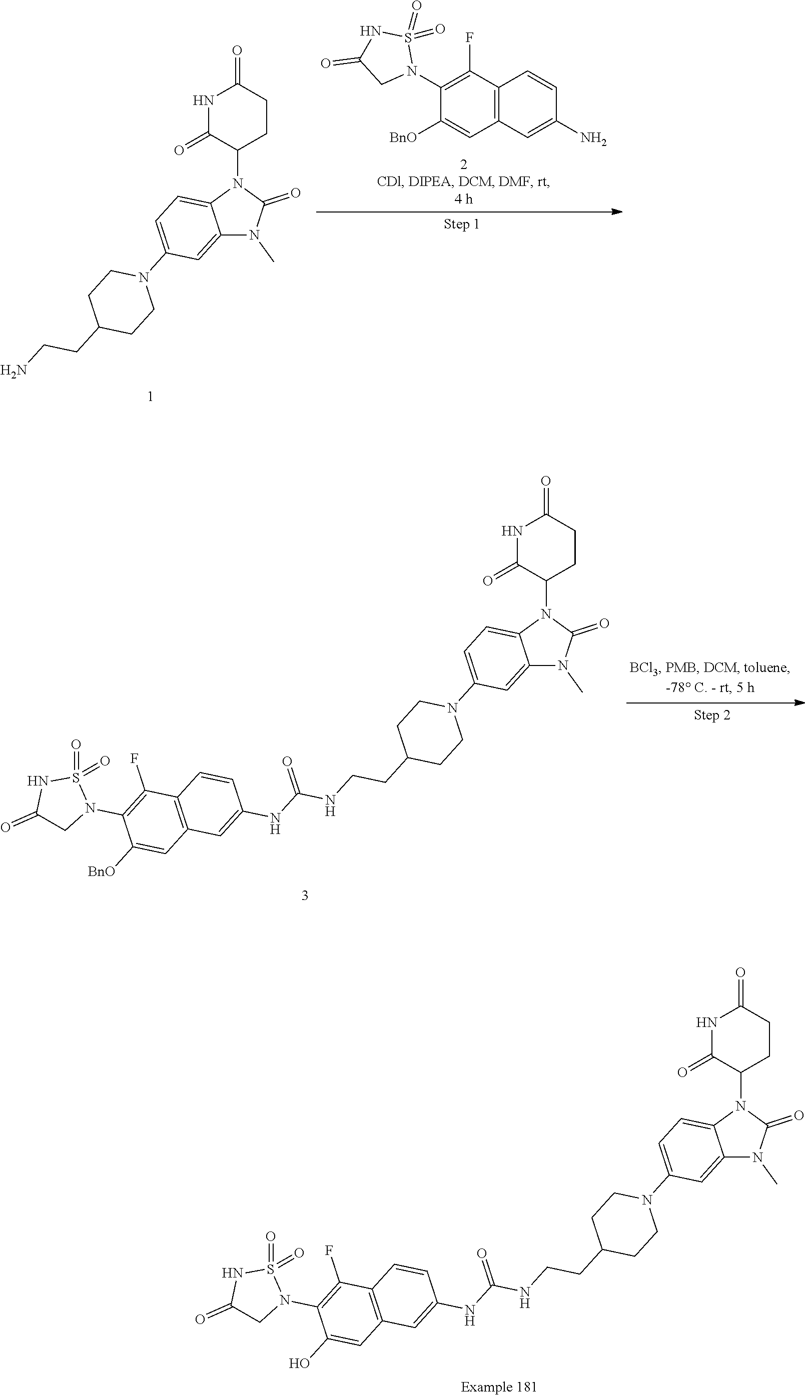 [SMILES: Cn1c(=O)n(C2CCC(=O)NC2=O)c2ccc(N3CCC(CCN)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(N3CCC(CCNC(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(N3CCC(CCNC(=O)Nc4ccc5c(F)c(N6CC(=O)NS6(=O)=O)c(O)cc5c4)CC3)cc21]

### Step 1: 1-[7-benzyloxy-5-fluoro-6-(1,1,4-trioxo-1,2,5-thiadiazolidin-2-yl)-2-naphthyl]-3-[2-[1-[1-(2,6-dioxo-3-piperidyl)-3-methyl-2-oxo-benzimidazol-5-yl]-4-piperidyl]ethyl]urea (3)

In to a 20 mL vial containing a well-stirred solution of 5-(6-amino-3-benzyloxy-1-fluoro-2-naphthyl)-1,1-dioxo-1,2,5-thiadiazolidin-3-one (188.89 mg, 423.51 μmol) in DCM (4 mL) were added DIPEA (296.80 mg, 2.30 mmol, 0.4 mL) and 1,1′-carbonyldiimidazole (103.01 mg, 635.26 μmol). After 2 h a solution of 3-[5-[4-(2-aminoethyl)-1-piperidyl]-3-methyl-2-oxo-benzimidazol-1-yl]piperidine-2,6-dione (164.89 mg, 423.51 μmol) in DMF (2 mL) was added. After 2 h, the reaction mixture was concentrated under reduced pressure and suspended in water (5 mL). The solid was filtered, washed with water and dried to afford 1-[7-benzyloxy-5-fluoro-6-(1,1,4-trioxo-1,2,5-thiadiazolidin-2-yl)-2-naphthyl]-3-[2-[[1-[1-(2,6-dioxo-3-piperidyl)-3-methyl-2-oxo-benzimidazol-5-yl]-4-piperidyl]ethyl]urea (220 mg, 117.83 μmol, 28% yield, 44% purity) as a brown solid. The material was used in the next step without purification. LCMS (ES+): m/z 813.0 [M+H]⁺

### Step 2: 1-[2-[[1-[1-(2,6-dioxo-3-piperidyl)-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Example 181)

In a 50 mL round bottom flask containing a well-stirred suspension of 1-[7-benzyloxy-5-fluoro-6-(1,1,4-trioxo-1,2,5-thiadiazolidin-2-yl)-2-naphthyl]-3-[2-[[1-[1-(2,6-dioxo-3-piperidyl)-3-methyl-2-oxo-benzimidazol-5-yl]-4-piperidyl]ethyl]urea (3, 210 mg, 114.96 μmol) in toluene (4 mL) and DCM (4 mL) was added pentamethylbenzene (85.21 mg, 574.82 μmol). The mixture was cooled to −78° C. and treated with boron trichloride (1.0 M in DCM) (269.41 mg, 2.30 mmol, 2.3 mL). The reaction mixture was stirred at RT for 5 h. The reaction mixture was cooled to −78° C. and quenched with 10% methanol in DCM (2 mL). The volatiles were removed under reduced pressure and the residue was triturated with diethyl ether. The material was purified by reverse phase prep HPLC [Purification method: Column: X Select C18 (150×19.1) mm, 5 microns; Mobile phase A: 0.1% TFA in water and Mobile phase B: MeCN] to afford 1-[2-[[1-[1-(2,6-dioxo-3-piperidyl)-3-methyl-2-oxo-benzimidazol-5-yl]-4-piperidyl]ethyl]-3-[5-fluoro-7-hydroxy-6-(1,1,4-trioxo-1,2,5-thiadiazolidin-2-yl)-2-naphthyl]urea (Example 181, 56 mg, 65.97 μmol, 57% yield, TFA salt) as a colorless solid. LCMS (ES+): m/z 723.0 [M+H]⁺

¹HNMR (400 MHZ, DMSO-d6): δ 11.11 (s, 1H), 9.83 (s, 1H), 8.76 (s, 1H), 7.90 (s, 1H), 7.76 (d, J=8.80 Hz, 1H), 7.28 (dd, J=2.00, 9.20 Hz, 1H), 7.13-7.10 (m, 2H), 6.88 (s, 1H), 6.33 (t, J=5.60 Hz, 1H), 5.55-5.25 (m, 1H), 4.18 (s, 2H), 3.63-3.46 (m, 4H), 3.35 (s, 3H), 3.23-3.20 (m, 2H), 2.92-2.86 (m, 1H), 2.72-2.61 (m, 3H), 2.03-1.95 (m, 3H), 1.52-1.44 (m, 5H).

### 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Example 182)

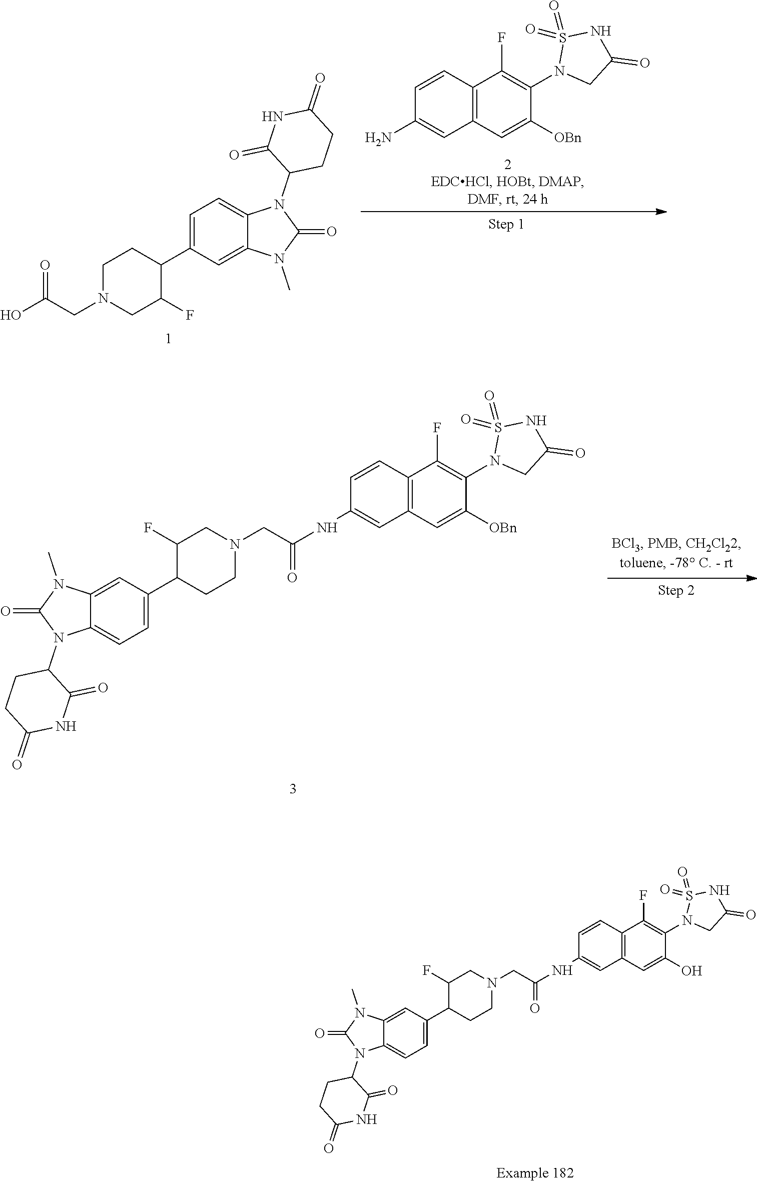 [SMILES: Cn1c(=O)n(C2CCC(=O)NC2=O)c2ccc(C3CCN(CC(=O)O)CC3F)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN(CC(=O)Nc4ccc5c(F)c(N6CC(=O)NS6(=O)=O)c(OCc6ccccc6)cc5c4)CC3F)cc21 ; Cn1c(=O)n(C2CCC(=O)NC2=O)c2ccc(C3CCN(CC(=O)Nc4ccc5c(F)c(N6CC(=O)NS6(=O)=O)c(O)cc5c4)CC3F)cc21]

### Step 1: N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-fluoro-1-piperidyl]acetamide (3)

Into a 50 mL single neck round bottom flask containing a well-stirred solution of 5-(6-amino-3-benzyloxy-1-fluoro-2-naphthyl)-1,1-dioxo-1,2,5-thiadiazolidin-3-one (1, 111.10 mg, 201.97 μmol, TFA salt) and 2-[4-[1-(2,6-dioxo-3-piperidyl)-3-methyl-2-oxo-benzimidazol-5-yl]-3-fluoro-1-piperidyl]acetic acid (2, 110 mg, 201.97 μmol, TFA salt) in anhydrous DMF (5 mL) were added EDC HCl (116.15 mg, 605.90 μmol). HOBt (54.58 mg, 403.94 μmol) and DMAP (148.04 mg, 1.21 mmol). After 24 h the solvent was removed under reduced pressure and the residue was suspended in 1.5 N aqueous HCl (70 mL). The resulting solid was filtered and dried to obtain N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-fluoro-1-piperidyl]acetamide (3, 130 mg, 135.03 μmol, 67% yield, HCl salt) as an off-white solid. The material was used in the next step without further purification. LCMS (ES+): m/z 802.0 [M+H]⁺

### Step 2: 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Example 182)

Into a 50 mL single neck round bottom flask containing a well-stirred solution of N-[7-benzyloxy-5-fluoro-6-(1,1,4-trioxo-1,2,5-thiadiazolidin-2-yl)-2-naphthyl]-2-[4-[1-(2,6-dioxo-3-piperidyl)-3-methyl-2-oxo-benzimidazol-5-yl]-3-fluoro-1-piperidyl]acetamide (3, 140 mg, 145.42 μmol, HCl salt) and pentamethylbenzene (107.79 mg, 727.08 μmol) in anhydrous DCM (2 mL) and toluene (2 mL) was added boron trichloride (1.0 M in DCM) (340.77 mg, 2.91 mmol, 2.91 mL) at −78° C. The reaction mixture was stirred at RT. After 2 h, the reaction was quenched with 5% MeOH in DCM (5 mL) at −78° C. The volatiles were removed under reduced pressure and the residue was triturated with MTBE (50 mL) and filtered. The material was purified by reverse phase column chromatography [Purification method: Silicycle C18 column; Mobile phase: 0.1% TFA in water; Mobile phase B: MeCN] to afford 2-[4-[1-(2,6-dioxo-3-piperidyl)-3-methyl-2-oxo-benzimidazol-5-yl]-3-fluoro-1-piperidyl]-N-[5-fluoro-7-hydroxy-6-(1,1,4-trioxo-1,2,5-thiadiazolidin-2-yl)-2-naphthyl]acetamide (Example 182, 100 mg, 117.50 μmol, 81% yield, TFA salt) as an off-white solid. LCMS (ES+): m/z 712.0 [M+H]⁺

¹HNMR (400 MHZ, DMSO-d₆): δ 11.13 (s, 1H), 10.74 (s, 1H), 10.18 (s, 1H), 8.14 (s, 1H), 7.93 (d, J=9.20 Hz, 1H), 7.51 (dd, J=1.20, 9.20 Hz, 1H), 7.14-7.12 (m, 2H), 7.02-7.00 (m, 2H), 5.41-5.37 (m, 1H), 5.19-5.08 (m, 1H), 4.26 (s, 2H), 3.50-3.43 (m, 1H), 3.01-3.30 (m, 2H), 3.38 (s, 3H), 2.97-2.88 (m, 3H), 2.77-2.70) (m, 3H), 2.09-2.01 (m, 4H).

### (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)-3-hydroxypropanamide (Example 183)

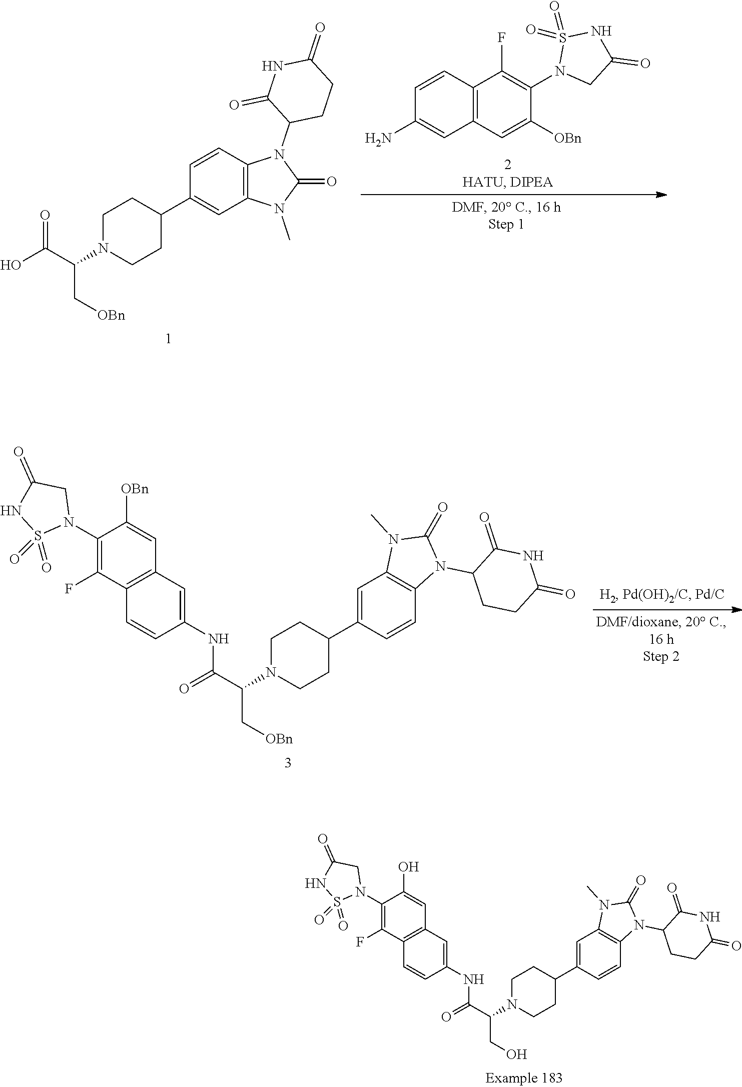 [SMILES: Cn1c(=O)n(C2CCC(=O)NC2=O)c2ccc(C3CCN([C@H](COCc4ccccc4)C(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN([C@H](COCc4ccccc4)C(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN([C@H](CO)C(=O)Nc4ccc5c(F)c(N6CC(=O)NS6(=O)=O)c(O)cc5c4)CC3)cc21]

### Step 1: (2R)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (3)

A mixture of (2R)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (1, 165 mg, 260.01 μmol, TFA salt), DIPEA (168.02 mg, 1.30 mmol, 226.44 μL) and HATU (109.33 mg, 286.01 μmol) in DMF (4 mL) was stirred for 15 min. 5-(6-amino-3-(benzyloxy)-1-fluoronaphthalen-2-yl)-1,2,5-thiadiazolidin-3-one 1,1-dioxide (2, 134.02 mg, 260.01 μmol, TFA salt) was added to the mixture, the mixture was stirred for 16 h. The reaction mixture was purified directly by prep-HPLC (flow: 25 mL/min; gradient: from 40-70% MeCN in water (0.1% TFA): column: Phenomenex Synergi C18 150×25 mm×10 μm) to afford (2R)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (3, 120 mg, 117.88 μmol, 45% yield, TFA salt) as white solid. LCMS (ESI): m/z 904.8 [M+H]⁺

### Step 2: (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)-3-hydroxypropanamide (Example 183)

To a solution of (2R)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (3, 105 mg, 103.14 μmol, TFA salt) in Dioxane (3 mL) and DMF (3 mL) was added Pd/C (20 mg, 10% purity) and Pd(OH)₂/C (20 mg, 10% purity). The mixture was stirred for 6 h under H₂ (15 psi) atmosphere. The mixture was filtered and concentrated under reduced pressure. The residue was purified by prep-HPLC (flow: 25 mL/min; gradient: from 60-90% MeCN in water (0.1% TFA); column: Phenomenex Synergi C18 150×25 mm×10 μm) to afford (2R)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)-3-hydroxypropanamide (Example 183, 37.95 mg, 44.39 μmol, 43% yield, TFA salt) as a white solid. LCMS (ESI): m/z 724.2 [M+H]⁺

¹H NMR (400 MHZ, DMSO-d₆) δ 11.10 (s, 1H), 10.81 (br s, 1H), 9.92 (br s, 1H), 9.80-9.65 (m, 1H), 8.19-8.08 (m, 1H), 7.91 (d, J=8.8 Hz, 1H), 7.50 (dd, J=1.6, 9.2 Hz, 1H), 7.09-7.00 (m, 3H), 6.94 (br d, J=8.6 Hz, 1H), 5.91-5.73 (m, 1H), 5.36 (dd, J=5.2, 12.4 Hz, 1H), 4.12 (s, 2H), 3.76 (dt, J=1.6, 4.2 Hz, 1H), 3.61-3.55 (m, 1H), 3.37-3.37 (m, 4H), 2.96-2.87 (m, 2H), 2.77-2.59 (m, 3H), 2.54-2.52 (m, 2H), 2.30-1.95 (m, 6H).

### (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)-3-hydroxypropanamide (Example 184)

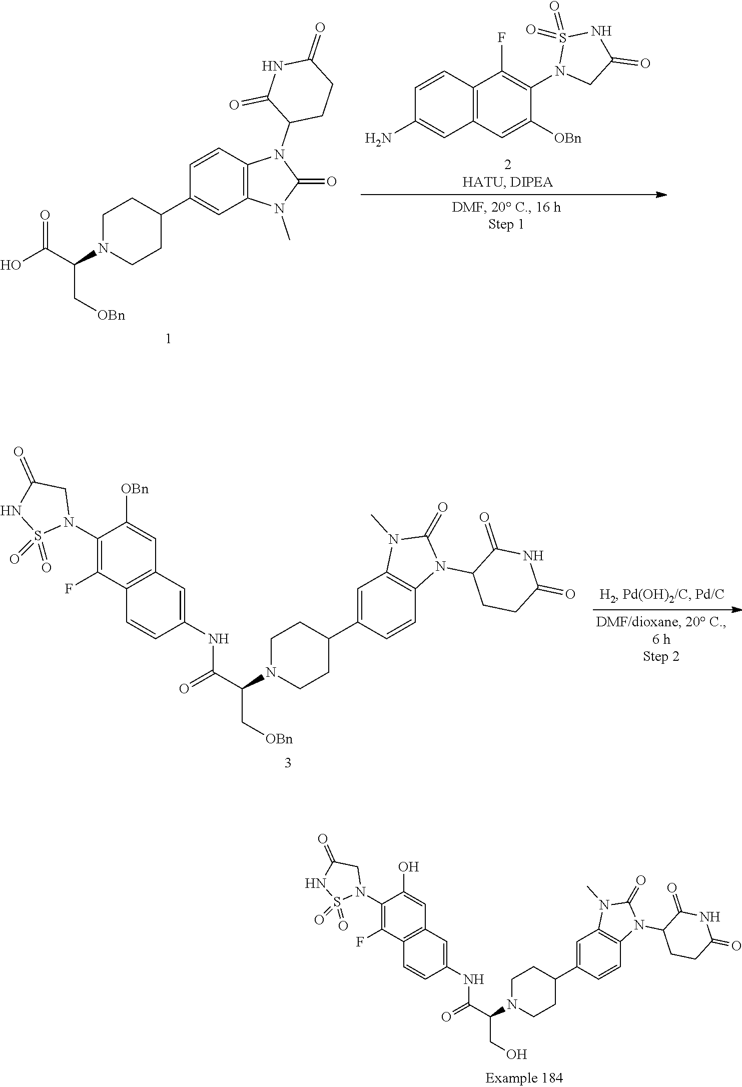 [SMILES: Cn1c(=O)n(C2CCC(=O)NC2=O)c2ccc(C3CCN([C@@H](COCc4ccccc4)C(=O)O)CC3)cc21 ; Nc1ccc2c(F)c(N3CC(=O)NS3(=O)=O)c(OCc3ccccc3)cc2c1 ; Cn1c(=O)n(C2CCC(=O)NC2=O)c2ccc(C3CCN([C@@H](COCc4ccccc4)C(=O)Nc4ccc5c(F)c(N6CC(=O)NS6(=O)=O)c(OCc6ccccc6)cc5c4)CC3)cc21 ; Cn1c(=O)n(C2CCC(=O)NC2=O)c2ccc(C3CCN([C@@H](CO)C(=O)Nc4ccc5c(F)c(N6CC(=O)NS6(=O)=O)c(O)cc5c4)CC3)cc21]

### Step 1: (2S)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (3)

A mixture of (2S)-3-(benzyloxy)-2-(4-(1-(2,6-dioxopiperidin-3-yl)-3-methyl-2-oxo-2,3-dihydro-1H-benzo[d]imidazol-5-yl)piperidin-1-yl)propanoic acid (1, 132 mg, 208.01 μmol, TFA salt), DIPEA (134.41 mg, 1.04 mmol, 181.15 μL) and HATU (87.46 mg, 228.81 μmol) in DMF (4 mL) was stirred for 15 min. 5-(6-amino-3-(benzyloxy)-1-fluoronaphthalen-2-yl)-1,2,5-thiadiazolidin-3-one 1,1-dioxide (2, 107.21 mg, 208.01 μmol, TFA salt) was added to the mixture and stirred for 16 h. The reaction mixture was purified by prep-HPLC (flow: 25 mL/min; gradient: from 40-70% MeCN in water (0.1% TFA): column: Phenomenex Synergi C18 150×25 mm×10 μm) to afford (2S)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (3, 101 mg, 99.21 μmol, 48% yield, TFA salt) as a white solid. LCMS (ESI): m/z 904.8 [M+H]⁺

### Step 2: (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)-3-hydroxypropanamide (Example 184)

To a solution of (2S)-3-(benzyloxy)-N-(7-(benzyloxy)-6-(1,1-dioxido-4-oxo-1,2,5-thiadiazolidin-2-yl)-5-fluoronaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)propanamide (3, 100 mg, 98.23 μmol, TFA salt) in 1,4-dioxane (3 mL) and DMF (3 mL) was added Pd/C (20 mg, 10% purity) and Pd(OH)₂/C (20 mg, 10% purity). The mixture was stirred for 6 h under H₂ (15 psi) atmosphere. The reaction mixture was filtered and concentrated under reduced pressure. The residue was purified by prep-HPLC (flow: 25 mL/min; gradient: from 62-92% MeCN in water (0.1% TFA); column: Phenomenex Synergi C18 150×25 mm×10 μm) to afford (2S)—N-(6-(1,1-dioxido-4-oxo-1,2,5-thiadiazolidin-2-yl)-5-fluoro-7-hydroxynaphthalen-2-yl)-2-(4-(1-(2,6-dioxopiperidin-3-yl)-3-methyl-2-oxo-2,3-dihydro-1H-benzo[d]imidazol-5-yl)piperidin-1-yl)-3-hydroxypropanamide (Example 184, 23.17 mg, 27.66 μmol, 28% yield, TFA salt) as an off-white solid. LCMS (ESI): m/z 724.5 [M+H]⁺

¹H NMR (400 MHZ, DMSO-d₆) δ 11.10 (s, 1H), 10.80 (br s, 1H), 9.92 (br s, 1H), 9.79-9.61 (m, 1H), 8.15 (s, 1H), 7.91 (d, J=8.8 Hz, 1H), 7.50 (br d, J=8.6 Hz, 1H), 7.09-7.01 (m, 3H), 6.94 (br d, J=8.6 Hz, 1H), 5.89-5.73 (m, 1H), 5.36 (br dd, J=5.0, 12.4 Hz, 1H), 4.12 (s, 2H), 3.78-3.75 (m, 1H), 3.57 (br s, 1H), 3.43-3.41 (m, 4H), 2.90 (br d, J=11.2 Hz, 2H), 2.75-2.63 (m, 4H), 2.56 (br d, J=5.8 Hz, 1H), 2.27-2.00 (m, 6H).

### N-(6-(1,1-dioxido-4-oxo-1,2,5-thiadiazolidin-2-yl)-5-fluoro-7-hydroxynaphthalen-2-yl)-2-(4-((3-(2,6-dioxopiperidin-3-yl)-1-methyl-1H-indazol-6-yl)amino)-3-methylphenyl)acetamide (Example 185)

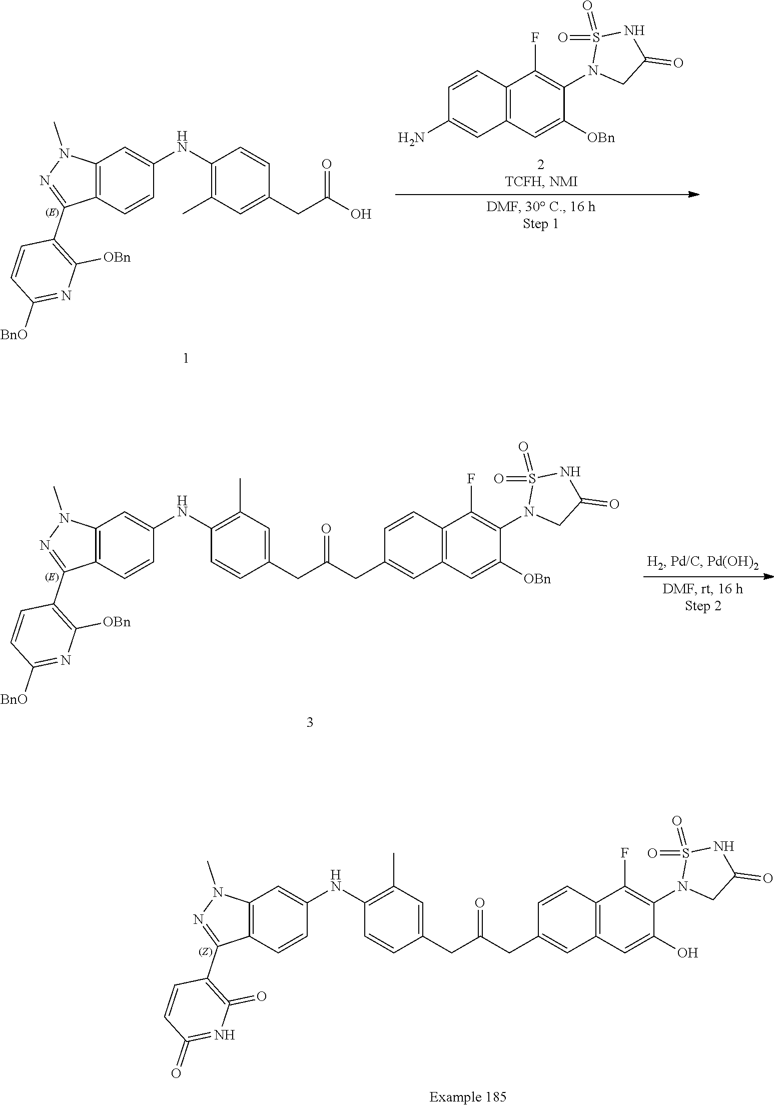

### Step 1: N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)-3-methylphenyl)acetamide (3)

To a solution of 2-(4-((3-(2,6-bis(benzyloxy)pyridin-3-yl)-1-methyl-1H-indazol-6-yl)amino)-3-methylphenyl)acetic acid (1, 250 mg, 427.60 μmol) and 5-(6-amino-3-(benzyloxy)-1-fluoronaphthalen-2-yl)-1,2,5-thiadiazolidin-3-one 1,1-dioxide (2, 205.97 mg, 513.12 μmol) in DMF (10 mL) were added 1-methylimidazole (175.53 mg, 2.14 mmol, 170.42 μL) and [chloro(dimethylamino)methylene]-dimethyl-ammonium; hexafluorophosphate (239.95 mg, 855.19 μmol). The mixture was stirred at 30° C. for 16 h. The mixture was filtered. The filtrate was purified by prep-HPLC (flow: 25 mL/min; gradient: from 25-55% MeCN in water (0.05% ammonia hydroxide v/v) over 10 min; column: Waters Xbridge C18 150×25 mm×5 μm) to afford N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)-3-methylphenyl)acetamide (3, 270 mg, 278.91 μmol, 65% yield) as a yellow solid. LCMS (ESI): m/z 968.4 [M+H]⁺

### Step 2: N-(6-(1,1-dioxido-4-oxo-1,2,5-thiadiazolidin-2-yl)-5-fluoro-7-hydroxynaphthalen-2-yl)-2-(4-((3-(2,6-dioxopiperidin-3-yl)-1-methyl-1H-indazol-6-yl)amino)-3-methylphenyl)acetamide (Example 185)

To a solution of N-(7-(benzyloxy)-6-(1,1-dioxido-4-oxo-1,2,5-thiadiazolidin-2-yl)-5-fluoronaphthalen-2-yl)-2-(4-((3-(2,6-bis(benzyloxy)pyridin-3-yl)-1-methyl-1H-indazol-6-yl)amino)-3-methylphenyl)acetamide (3, 240 mg, 247.92 μmol) in DMF (16 mL) were added Pd/C (50 mg, 10% purity) and Pd(OH)₂ (50 mg, 10% purity). The mixture was stirred at 30° C. for 16 h under H₂ (15 psi) atmosphere. The mixture was filtered and the filtrate was purified by prep-HPLC (flow: 25 mL/min; gradient: from 32-62% MeCN in water (0.1% TFA) over 10 min; column: 3 Phenomenex luna C18 75×30 mm×3 μm) to afford N-(6-(1,1-dioxido-4-oxo-1,2,5-thiadiazolidin-2-yl)-5-fluoro-7-hydroxynaphthalen-2-yl)-2-(4-((3-(2,6-dioxopiperidin-3-yl)-1-methyl-1H-indazol-6-yl)amino)-3-methylphenyl)acetamide (Example 185, 110 mg, 133.83 μmol, 54% yield, TFA salt) as an off-white solid. LCMS (ESI): m/z 700.1 [M+H]⁺

¹H NMR (400 MHZ, DMSO-d₆) δ 10.84 (s, 1H), 10.39 (s, 2H), 8.19 (s, 1H), 7.86 (d, J=8.8 Hz, 1H), 7.53-7.42 (m, 2H), 7.28-7.20 (m, 2H), 7.19-7.11 (m, 1H), 6.96 (s, 1H), 6.78 (dd, J=1.6, 8.4 Hz, 1H), 6.64 (d, J=1.2 Hz, 1H), 4.37 (s, 2H), 4.24 (dd, J=4.8, 9.2 Hz, 1H), 3.77 (s, 3H), 3.64 (s, 2H), 2.69-2.57 (m, 2H), 2.32-2.25 (m, 1H), 2.22 (s, 3H), 2.19-2.10 (m, 1H).

The structures shown in Table 2 were synthesized using methods similar to those described for Examples 1-185

### HiBit Method

### Materials

Dulbecco's modified Eagle medium (DMEM) without phenol red and fetal bovine serum (FBS) were purchased from Gibco (Grand Island, NY, USA). Nano-Glo® HiBIT Lytic Assay System was purchased from Promega (Madison, WI, USA). 293T.109 (HiBiT-PTPN2) cell line was generated by ectopically expressing PTPN2 with N-terminal HiBiT fusion tag in 293T WT cell line purchased from ATCC (Manassas, VA, USA). Cell culture flasks and 384-well microplates were acquired from VWR (Radnor, PA, USA).

### PTPN2 Degradation Analysis

PTPN2 degradation was determined based on quantification of luminescent signal using Nano-Glo® HiBIT Lytic Assay kit. Test compounds were added to the 384-well plate from a top concentration of 10 μM with 11 points, half log titration in duplicates. 293T.109 cells were added into 384-well plates at a cell density of 5000 cells per well. The plates were kept at 37° C. with 5% CO₂ for 24 hours. The cells treated in the absence of the test compound were the negative control and the cells without Nano-Glo® HiBiT Lytic reagent were the positive control. After 24-hour incubation, Nano-Glo® HiBIT Lytic Assay reagents were added to the cells. Luminescence was acquired on EnVision™ Multilabel Reader (PerkinElmer, Santa Clara, CA, USA).

TABLE B1

| | | |
|---|---|---|
| | HiBiT-Degradation | HiBiT-Degradation |
| | 293T.109 PTPN2 | B16F10.4 PTPN2 |
| Ex. | 24.0 hours | 24.0 hours |

| No. | DC₅₀ [nM] | Eₘₐₓ | DC₅₀ [nM] | Eₘₐₓ |
|---|---|---|---|---|
| 1 | D | A | E | B |
| 2 | D | B | D | A |
| 3 | C | A | D | A |
| 4 | C | A | E | C |
| 5 | C | B | E | D |
| 6 | C | B | E | C |
| 7 | B | B | B | B |
| 8 | B | A | C | B |
| 9 | C | B | D | C |
| 10 | C | B | E | C |
| 11 | D | A | D | A |
| 12 | D | B | E | C |
| 13 | C | A | D | C |
| 14 | D | B | D | C |
| 15 | D | A | D | B |
| 16 | C | C | D | C |
| 17 | C | A | E | C |
| 18 | D | B | E | C |
| 19 | C | A | D | C |
| 20 | C | A | C | B |
| 21 | C | B | D | C |
| 22 | A | A | C | A |
| 23 | B | A | C | A |
| 24 | C | A | C | A |
| 25 | D | B | E | C |
| 26 | C | A | C | A |
| 27 | B | A | D | B |
| 28 | A | A | C | A |
| 29 | A | A | C | A |
| 30 | E | C | E | D |
| 31 | D | B | E | C |
| 32 | D | B | D | B |
| 33 | D | C | E | C |
| 34 | E | D | E | E |
| 35 | D | B | D | B |
| 36 | D | B | D | B |
| 37 | E | C | E | D |
| 38 | E | C | E | E |
| 39 | E | C | E | C |
| 40 | D | B | E | B |
| 41 | D | B | E | C |
| 42 | D | B | E | C |
| 43 | E | C | E | E |
| 44 | E | C | E | D |
| 45 | D | C | E | B |
| 46 | E | C | E | C |
| 47 | E | E | E | E |
| 48 | E | C | E | D |
| 49 | E | E | | |
| 50 | E | E | | |
| 51 | E | D | | |
| 52 | E | C | E | C |
| 53 | D | C | E | D |
| 54 | D | C | E | C |
| 55 | D | A | E | B |
| 56 | E | C | D | B |
| 57 | D | A | E | C |
| 58 | D | B | E | C |
| 59 | D | B | D | C |
| 60 | D | B | D | C |
| 61 | D | B | E | C |
| 62 | D | B | E | C |
| 63 | D | B | E | C |
| 64 | D | B | E | C |
| 65 | D | B | D | B |
| 66 | D | B | E | D |
| 67 | D | B | E | B |
| 68 | D | C | E | C |
| 69 | E | C | E | D |
| 70 | E | D | E | E |
| 71 | E | E | E | E |
| 72 | B | A | C | A |
| 73 | B | A | B | A |
| 74 | A | A | C | B |
| 75 | C | B | C | B |
| 76 | B | A | C | B |
| 77 | B | A | B | A |
| 78 | B | A | B | A |
| 79 | B | A | D | B |
| 80 | B | B | C | B |
| 81a | B | A | B | A |
| 81b | B | B | D | C |
| 82 | A | A | D | C |
| 83 | A | A | B | A |
| 84 | B | A | C | B |
| 85 | C | B | D | C |
| 86 | B | A | C | A |
| 87 | B | A | B | A |
| 88 | C | B | C | A |
| 89 | C | B | C | B |
| 90 | C | B | D | B |
| 91 | B | C | C | B |
| 92 | C | A | D | B |
| 93 | B | B | C | C |
| 94 | D | B | D | C |
| 95 | B | C | C | B |
| 96 | C | C | C | B |
| 97 | C | A | D | B |
| 98 | B | A | C | A |
| 99 | C | B | C | A |
| 100 | D | C | D | B |
| 101 | D | B | E | B |
| 102 | D | C | D | C |
| 103 | D | C | C | B |
| 104 | C | B | C | B |
| 105 | B | A | B | A |
| 106 | D | B | D | C |
| 107 | C | C | C | C |
| 108 | D | C | E | C |
| 109 | D | C | C | A |
| 110 | B | A | C | A |
| 111 | B | C | C | C |
| 112 | B | B | C | A |
| 113 | D | B | D | A |
| 114 | C | B | C | B |
| 115 | D | C | D | B |
| 116 | C | B | C | B |
| 117 | B | A | C | A |
| 118 | C | A | B | A |
| 119 | B | A | C | B |
| 120 | B | A | C | B |
| 121 | B | B | C | B |
| 122 | B | A | D | B |
| 123 | B | B | C | A |
| 124 | D | C | D | C |
| 125 | C | B | D | A |
| 126 | D | C | D | C |
| 127 | C | C | D | B |
| 128 | C | B | D | B |
| 129 | C | A | C | A |
| 130 | C | B | C | A |
| 131 | B | A | C | A |
| 132 | C | B | D | C |
| 133 | B | B | C | A |
| 134 | C | C | D | C |
| 135 | C | C | C | B |
| 136 | B | A | C | A |
| 137 | C | C | C | C |
| 138 | C | B | C | A |
| 139 | C | B | C | A |
| 140 | C | C | E | D |
| 141 | B | A | C | A |
| 142 | C | C | D | C |
| 143 | D | C | D | B |
| 144 | B | A | C | A |
| 145 | B | A | C | A |
| 146 | B | A | D | B |
| 147 | D | C | D | C |
| 148 | D | B | D | B |
| 149 | B | B | C | B |
| 150 | C | B | C | B |
| 151 | C | B | E | C |
| 152 | B | A | D | B |
| 153 | C | C | D | C |
| 154 | D | C | E | D |
| 155 | C | B | D | C |
| 156 | B | A | D | A |
| 157 | B | B | C | A |
| 158 | B | B | C | A |
| 159 | C | A | C | A |
| 160 | B | A | D | B |
| 161 | D | B | D | C |
| 162 | B | B | C | B |
| 163 | B | B | B | A |
| 164 | B | B | C | A |
| 165 | B | C | C | C |
| 166 | C | C | D | C |
| 167 | C | A | C | A |
| 168 | C | A | B | B |
| 169 | B | A | B | A |
| 170 | B | A | C | A |
| 171 | C | A | C | A |
| 172 | B | A | B | B |
| 173 | B | B | C | B |
| 174 | B | A | C | A |
| 175 | B | A | C | A |
| 176 | B | A | C | A |
| 177 | B | A | C | A |
| 178 | B | A | C | A |
| 179 | B | A | D | B |
| 180 | B | A | C | B |
| 181 | B | A | C | A |
| 182 | B | A | B | A |
| 183 | B | A | B | A |
| 184 | B | A | C | A |
| 185 | B | A | C | A |

DC₅₀ values: A: DC₅₀ < 10 nM; B: 10 nM ≤ DC₅₀ < 50 nM; C: 50 nM ≤ DC₅₀ < 200 nM; D: 200 nM ≤ DC₅₀ < 1000 nM; E: 1000 nM ≤ DC₅₀;
Eₘₐₓ values: A: Eₘₐₓ < 10; B: 10 ≤ Eₘₐₓ < 20; C: 20 ≤ Eₘₐₓ < 50; D: 50 ≤ Eₘₐₓ < 80; E: 80 ≤ Eₘₐₓ < 100

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 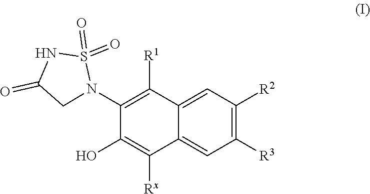
or a pharmaceutically acceptable salt thereof:
wherein:
R¹ is hydrogen or halogen;
R² is hydrogen, halogen, C1-C3 alkoxy, C3-C6 cycloalkoxy, C1-C3 haloalkoxy, C3-C5 halocycloalkoxy, C1-C3 alkyl, C1-C3 haloalkyl, C3-C6 cycloalkyl, or -L-Z;
R³ is hydrogen, halogen, C1-C3 alkoxy, C3-C5 cycloalkoxy, C1-C3 haloalkoxy, C3-C5 halocycloalkoxy, C1-C3 alkyl, C1-C3 haloalkyl, C3-C5 cycloalkyl, or -L-Z;
wherein one of R² and R³ is -L-Z and the other of R² and R³ is not -L-Z;
R^{x} is hydrogen or halogen;
L is —U—V—W—X—Y—;
U is a bond, —(NR⁴)—, —O—, C1-C3 alkylene, C2-C3 alkenylene, C2-C3 alkynylene, C3-C6 cycloalkylene, 4-10 membered heterocyclylene, 5-10 membered heteroarylene, —(C═O)NR⁴—, —NR⁴(C═O)—, —OR⁵—, —R⁵O—, —NR+R⁵—, —R⁵NR⁴—, or —(NR⁴)(C═O)(NR⁴);
each R⁴ is independently a hydrogen, C1-C6 alkyl, or C3-C5 cycloalkyl;
R⁵ is C1-C3 alkylene, C3-C7 cycloalkylene, or 4-12 membered heterocyclylene;
V is a bond, —(NR⁴), —O—, C1-C6 alkylene, C2-C6 alkenylene, (C═O)NR⁴—, —(NR⁴)R⁵—, —(NR⁴)(C═O)—, —NH(C═O)NH—, —OR⁵—, —R⁵O—, 4-10-membered heterocyclylene, 5-10 membered heteroarylene, C6-C10 arylene, or C3-C6 cycloalkylene;
W is a bond, C1-C3 alkylene optionally substituted with hydroxyl, C3-C6 cycloalkylene, 4-12 membered heterocyclylene, —O—, —(NR⁴)—, —R⁵(NR⁴), (NR⁴)R⁵—, —(NR⁴)(C═O)—, —R⁵(NR⁴)(C═O)—, —(C═O)(NR⁴)R⁵—, —R⁵(C═O)(NR⁴)—, —(C═O)(NR⁴)—, —R⁵(C═O)—, —(C═O)R⁵—, —(C═O)—, —(S═O)—, or —S(O₂)—;
X is a bond, C1-C3 alkylene, C3-C6 cycloalkylene, 4-12 membered heterocyclylene, C6-C10 arylene, 5-10 membered heteroarylene, —R⁵(NR⁴)(C═O)—, —(C═O)R′(NR⁴)—, —R⁵(C═O)(NR⁴)—, —(NR⁴)(C═O)R⁵—, —R⁵(C═O)(NR⁴)—, —(C═O)(NR⁴)R⁵—, —(NR⁴)R⁵(C═O)—, —R⁵(C═O)(NR⁴)R⁵—, —R⁵(NR⁴)(C═O)R⁵—, —(C═O)R⁵—, or —R⁵(C═O)—;
Y is R⁶, R⁶(CR^{A}R^{B})ₚ-Q-, or -Q (CR^{A}R^{B})ₚR⁶—;
Q is —(NR⁴)—, —O—, or —(CR^{A}R^{B})ₚ—;
p is 0, 1, 2, or 3;
R⁶ is C1-C3 alkylene, C3-C7 cycloalkylene, 4-12 membered heterocyclylene, C6-C10 arylene, or 5-10 membered heteroarylene;
wherein the heterocyclylene, heteroarylene, arylene, and cycloalkylene groups of U, V, W, X, and R⁶ are each optionally substituted with 1-3 substituents independently selected from fluoro, hydroxyl, C1-C6 alkoxy, and C1-C6 alkyl;
each R^{A} and R^{B} is independently hydrogen, fluoro, or C1-C6 alkyl; or
R^{A} and R^{B}, together with the carbon atom to which they are attached, come together to form a C3-C4 cycloalkyl; or
R^{A} and R^{B} combine to form oxo;
Z is selected from the group consisting of 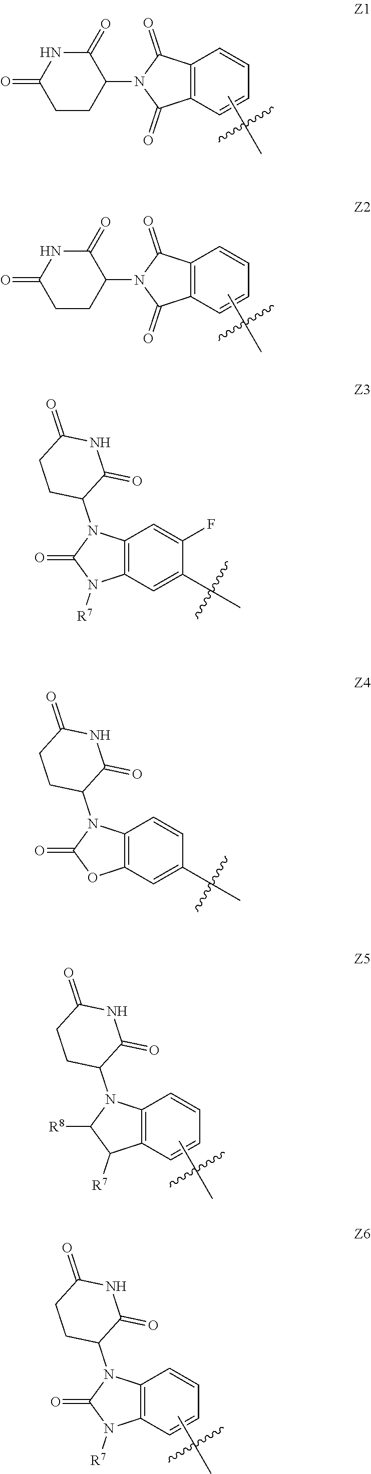 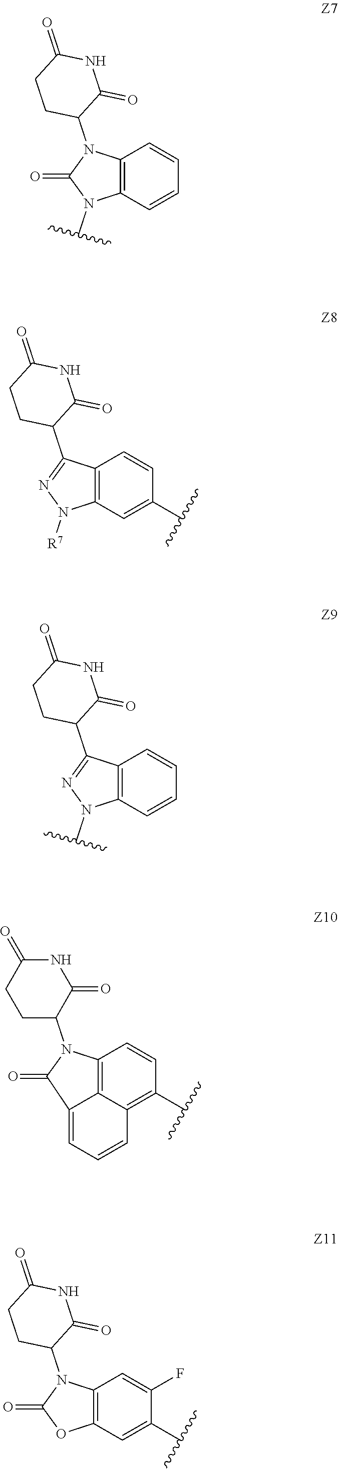 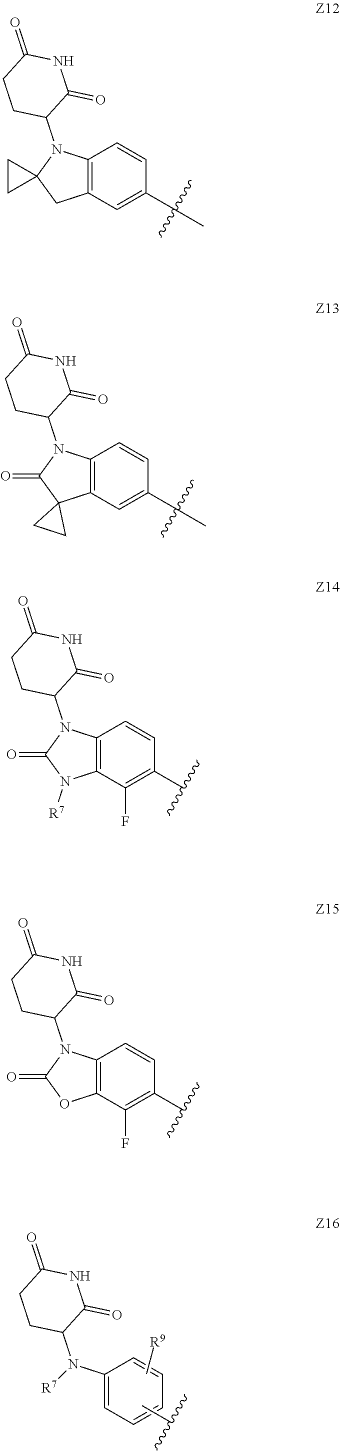 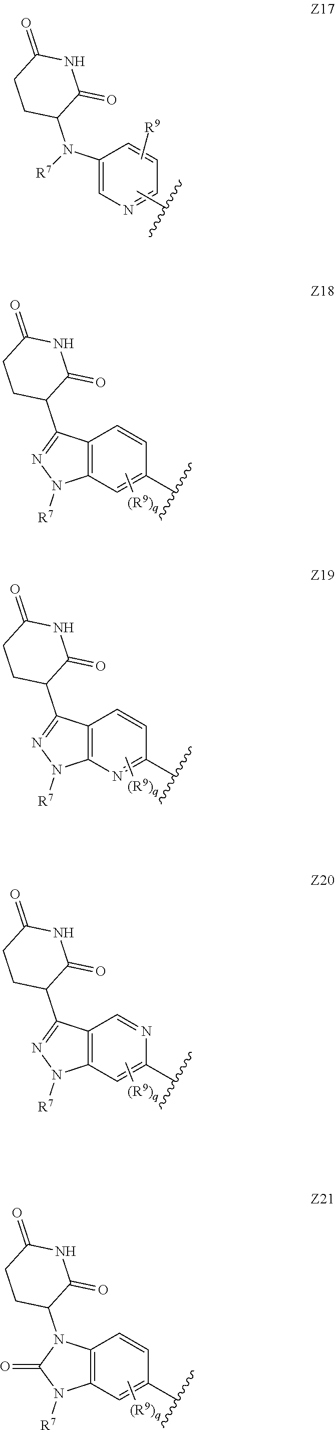 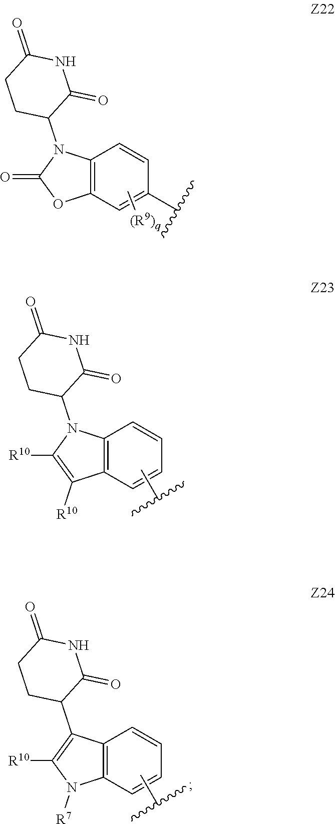
R⁷ is hydrogen, C1-C6 alkyl optionally substituted with one group selected from hydroxyl, cyano and C1-C6 alkoxy, C1-C6 haloalkyl, C3-C6 cycloalkyl, 4-6 membered heterocyclyl, —(CR^{A}R^{B})(4-12 membered heterocyclyl), or —(CR^{A}R^{B})(C3-C6 cycloalkyl);
R⁸ is hydrogen or C1-C6 alkyl; and
each R⁹ is hydrogen, halogen, cyano, C1-C6 alkyl, C1-C6 haloalkyl, C1-C6 alkoxy, C1-C5 cycloalkoxy, 5-10 membered heteroaryloxy, or phenoxy;
q is 0, 1, or 2; and
each R¹⁰ is independently hydrogen, halogen, cyano, C1-C6 alkyl, C3-C6 cycloalkyl, or C1-C6 haloalkyl.

2-124. (canceled)

125. The compound of claim 1, wherein;
(i) U is —NR⁴(C═O)— or —(C═O)NR⁴—; V is a bond or C1-C6 alkylene;
W is a bond; and X is a bond;
(ii) U is —NR⁴(C═O)— or —(C═O)NR⁴—; V is a bond or C1-C6 alkylene;
W is a bond; and X is 4-12-membered heterocyclylene;
(iii) U is —O—; V is C1-C6 alkylene, C3-C6 cycloalkylene, or 4-10-membered heterocyclylene; W is —C(═O)—, —N(R⁴)—, —C(═O)NR⁴—, —NR⁴C(═O)—, or —NR⁴C(═O)R⁵—;
(iv) U is —NR⁴—; Vis C1-C6 alkylene or a bond; W is —C(═O)— or —C(═O)R⁵—; and X is a bond;
(v) U is a bond, C1-C3 alkylene, C2-C3 alkenylene, or C2-C3 alkynylene; V is a bond; W is a bond or C(═O); and X is a bond or C6-C10 arylene; or
(vi) U is —NR⁴(C═O)— or —(C═O)NR⁴—; V is a bond; W is C1-C3 alkylene; and X is a bond.

126-156. (canceled)

157. The compound of claim 1, wherein;
Y is R⁶;
Y is —R⁶(CR^{A}R^{B})ₚ-Q-;
Y is —R⁶(CR^{A}R^{B})ₚ-Q-, where p is 0;
Y is R⁶(CR^{A}R^{B})ₚ-Q-, where p is 1 or 2, and each R^{A} and R^{B} are hydrogen;
Y is —R⁶(CR^{A}R^{B})ₚ-Q-, where p is 1 or 2, and each R^{A} and R^{B} are independently hydrogen or C1-C3 alkyl, or one pair of R^{A} and R^{B}, together with the carbon atom to which they are attached, come together to form a C3-C4 cycloalkyl, and each remaining R^{A} and R^{B}, if present, are hydrogen; or
Y is —R⁶C(═O)(CR^{A}R^{B})-Q-, where each R^{A} and R^{B} are independently hydrogen, fluoro, or C1-C3 alkyl.

158-226. (canceled)

227. The compound of claim 1, wherein;
(i) Z is: 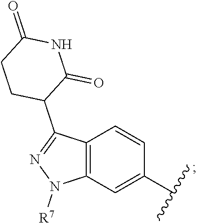
(ii) Z is selected from the group consisting of; 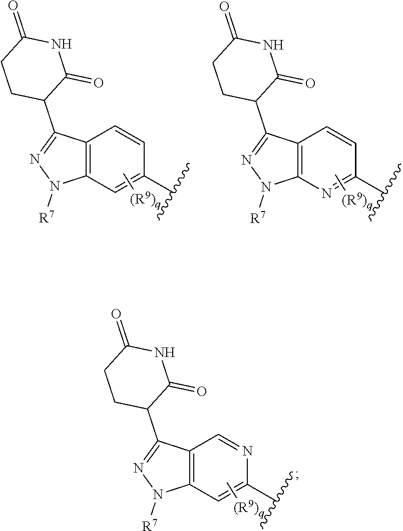
(iii) Z is: 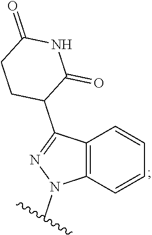 [SMILES: *n1nc(C2CCC(=O)NC2=O)c2ccccc21]
(iv) Z is 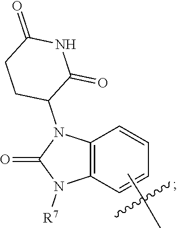
(v) Z is selected from the group consisting of: 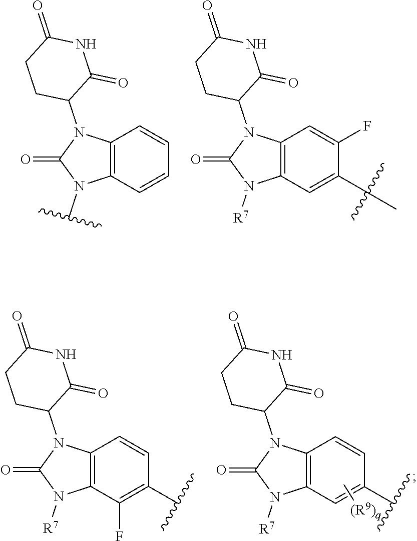
(vi) Z is: 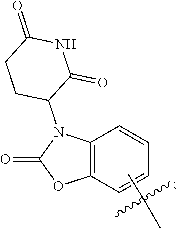
(vii) Z is selected from the group consisting of: 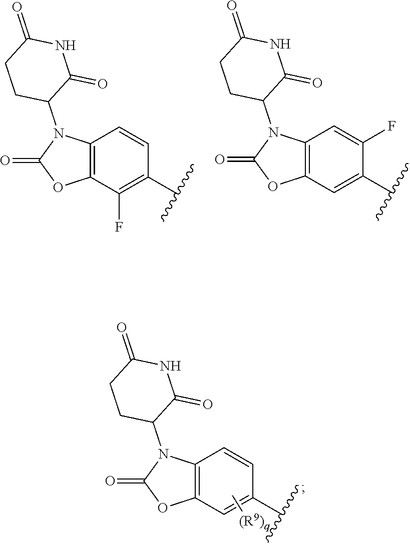
(viii) Z is selected from the group consisting of: 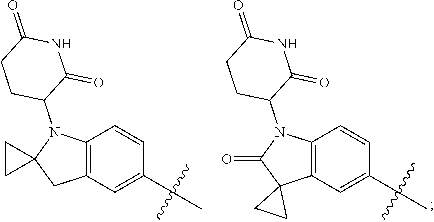 [SMILES: *c1ccc2c(c1)CC1(CC1)N2C1CCC(=O)NC1=O ; *c1ccc2c(c1)C1(CC1)C(=O)N2C1CCC(=O)NC1=O]
(ix) Z is selected from the group consisting of: 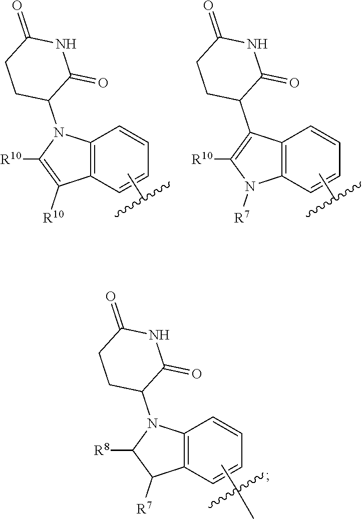
(x) Z is 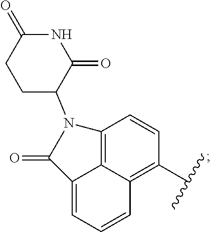 [SMILES: *c1ccc2c3c(cccc13)C(=O)N2C1CCC(=O)NC1=O]
(xi) Z is 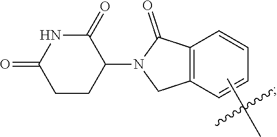
(xii) Z is 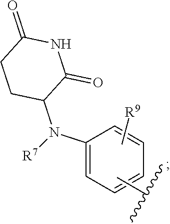
or
(xiii) Z is

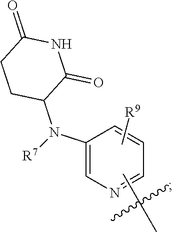

228-268. (canceled)

269. The compound of claim 1, wherein the compound of Formula (I) is a compound of Formula (I-a): 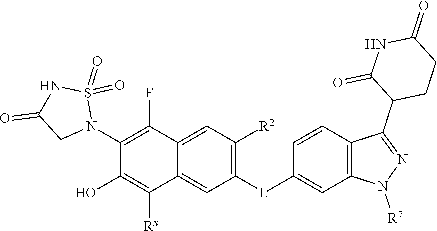
or a pharmaceutically acceptable salt thereof.

270. The compound of claim 1, wherein the compound of Formula (I) is a compound of Formula (I-b): 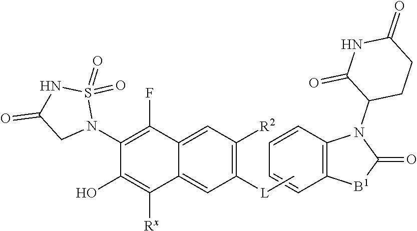
or a pharmaceutically acceptable salt thereof;
wherein B¹ is O or NR⁷.

271-278. (canceled)

279. The compound of claim 1, wherein the compound of Formula (I) is a compound of Formula (I-c): 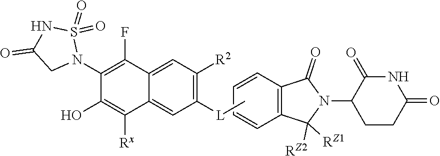
or a pharmaceutically acceptable salt thereof;
wherein R^{Z1} and R²² are both hydrogen; or R^{Z1} and R^{Z2} combine to form oxo.

280-283. (canceled)

284. The compound of claim 1, wherein the compound of Formula (I) is a compound of Formula (I-d): 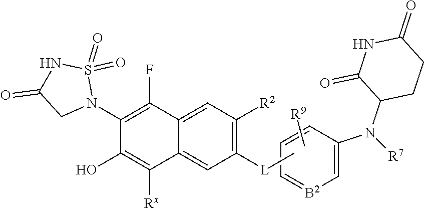
or a pharmaceutically acceptable salt thereof,
wherein B² is CH or N.

285-289. (canceled)

290. The compound of claim 1, wherein the compound of Formula (I) is a compound of Formula (I-e): 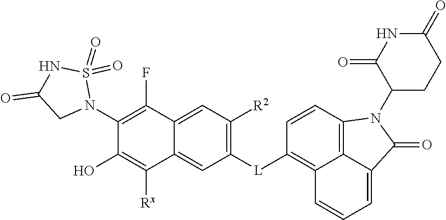
or a pharmaceutically acceptable salt thereof.

291-294. (canceled)

295. The compound of claim 1, wherein the compound of Formula (I) is a compound of Formula (II-a): 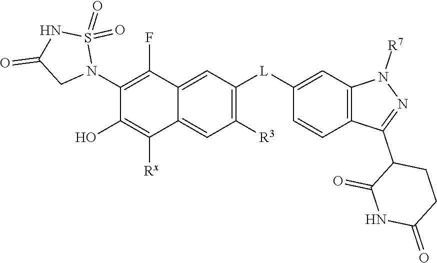
or a pharmaceutically acceptable salt thereof.

296. The compound of claim 1, wherein the compound of Formula (I) is a compound of Formula (II-b): 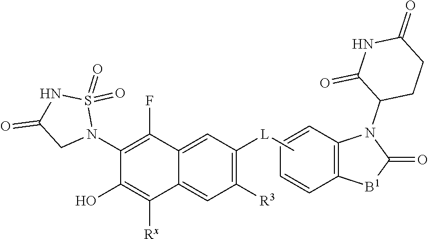
or a pharmaceutically acceptable salt thereof;
wherein B¹ is O or NR⁷.

297-304. (canceled)

305. The compound of claim 1, wherein the compound of Formula (I) is a compound of Formula (II-c): 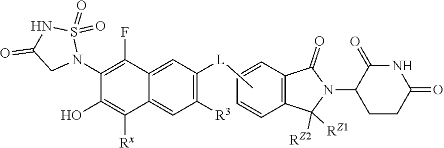
or a pharmaceutically acceptable salt thereof;
wherein R^{Z1} and R²² are both hydrogen; or R^{Z1} and R²² combine to form oxo.

306-309. (canceled)

310. The compound of claim 1, wherein the compound of Formula (I) is a compound of Formula (II-d): 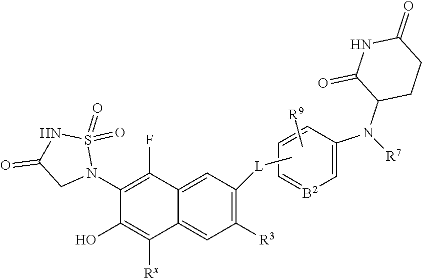
or a pharmaceutically acceptable salt thereof,
wherein B² is CH or N.

311-315. (canceled)

316. The compound of claim 1, wherein the compound of Formula (I) is a compound of Formula (II-e): 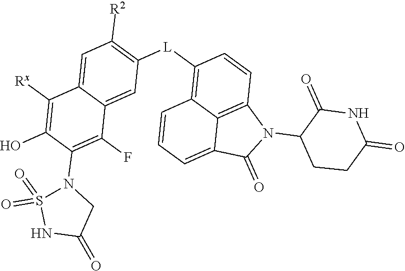
or a pharmaceutically acceptable salt thereof.

317-413. (canceled)

414. The compound of claim 1, wherein;
(i) L is selected from the group consisting of: 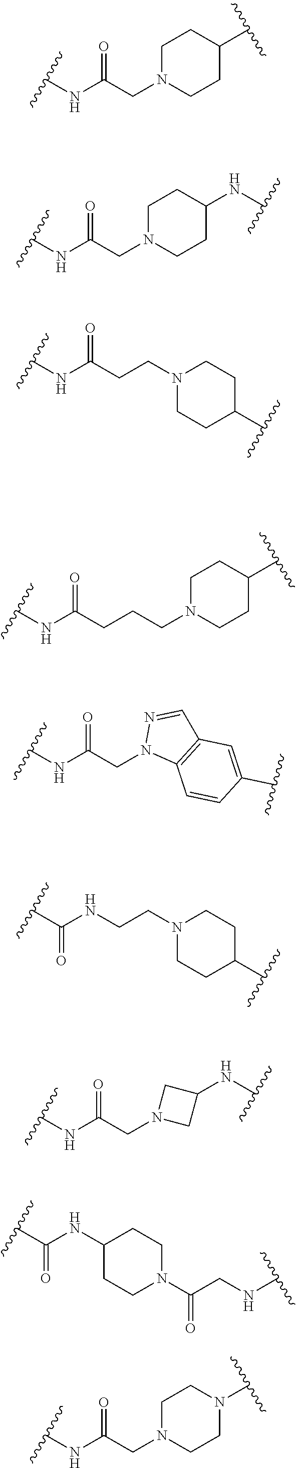 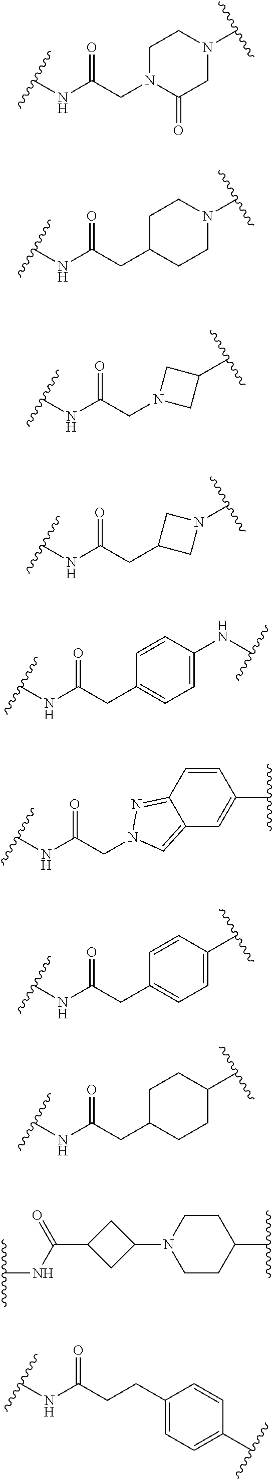 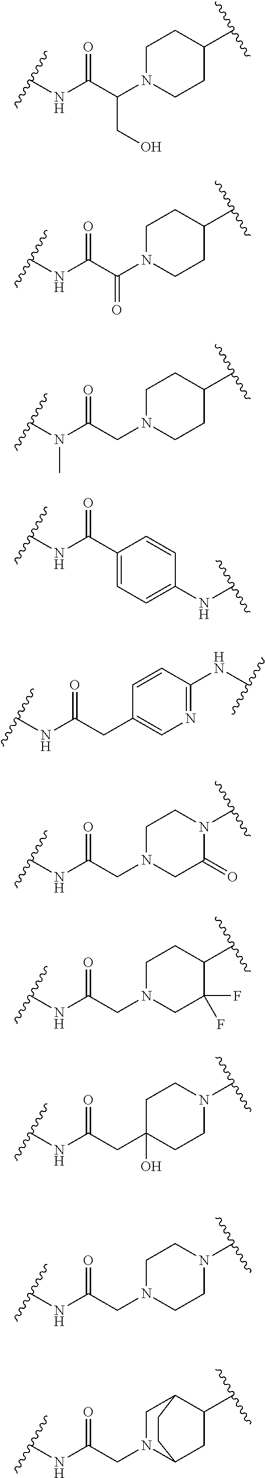 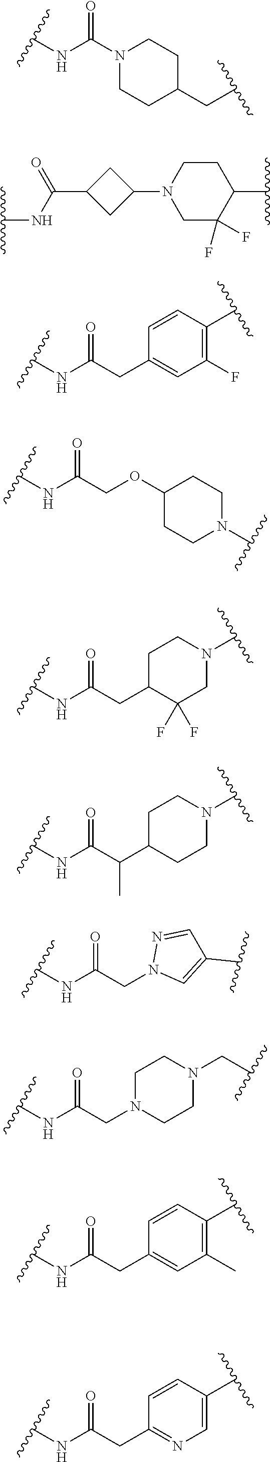 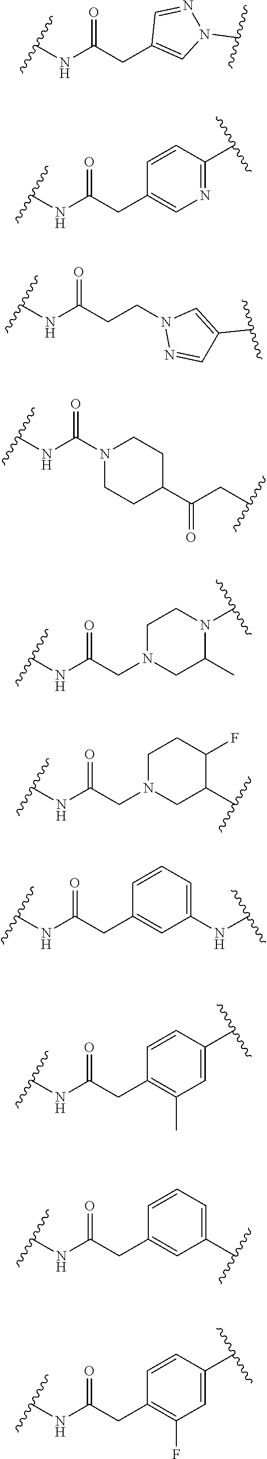 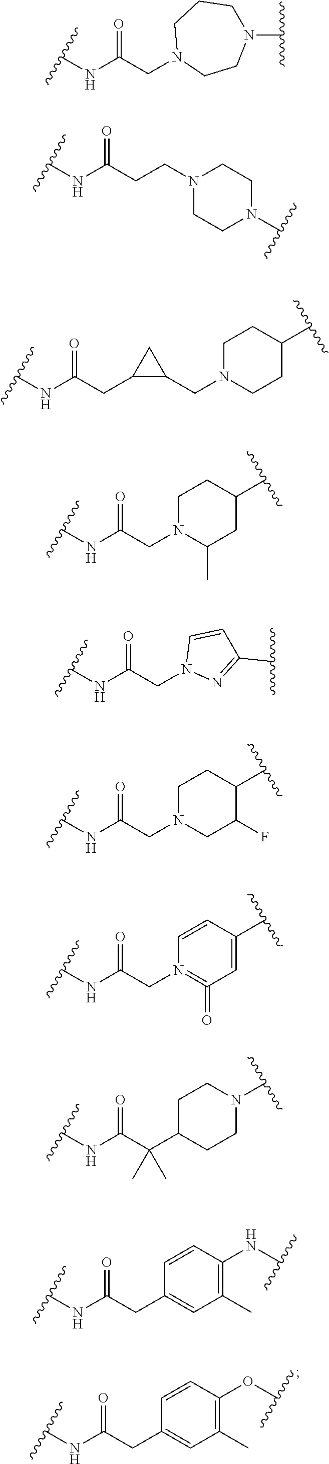
(ii) L is selected from the group consisting of: 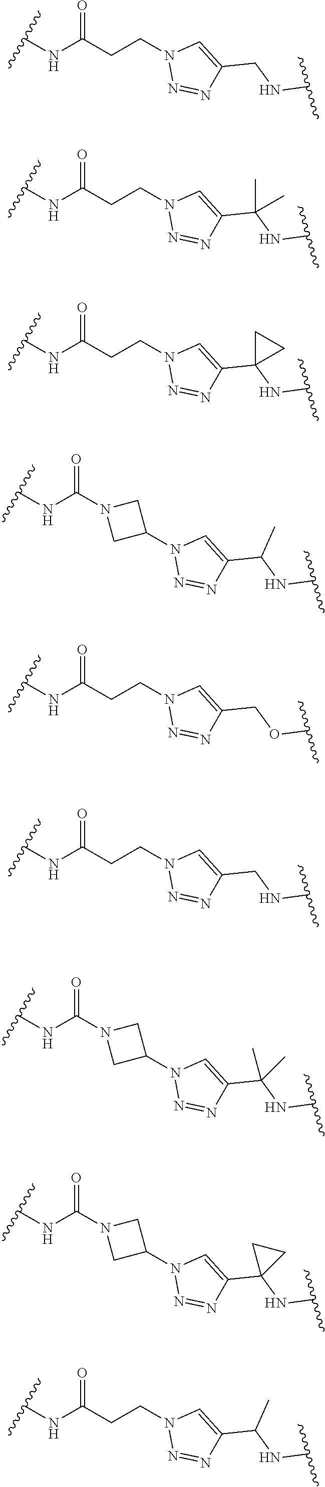 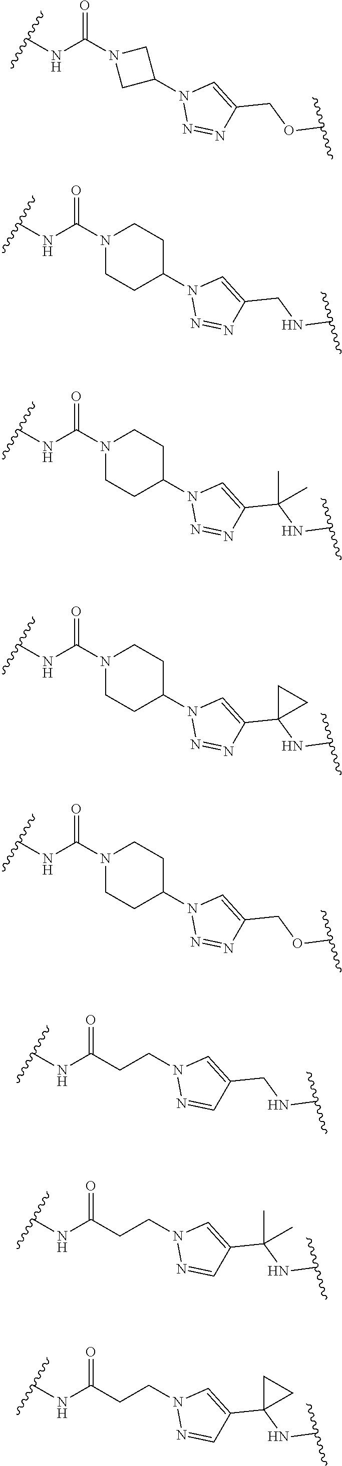 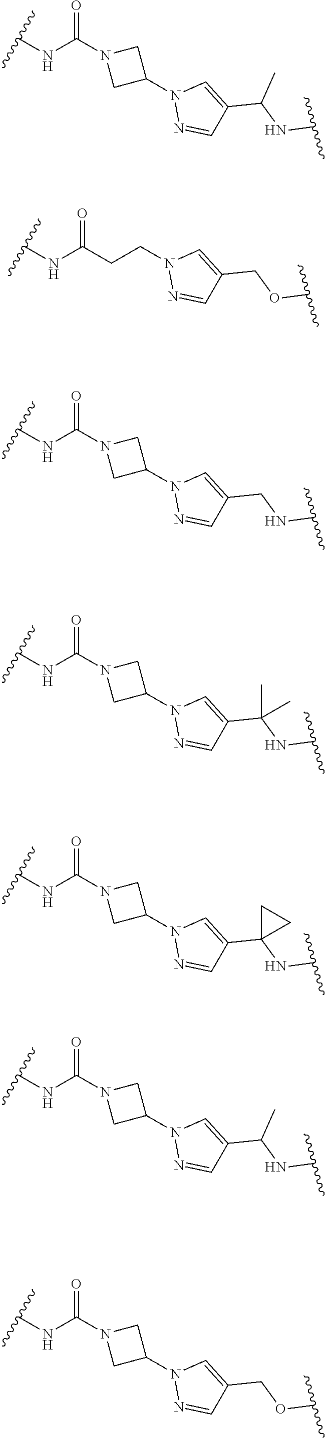 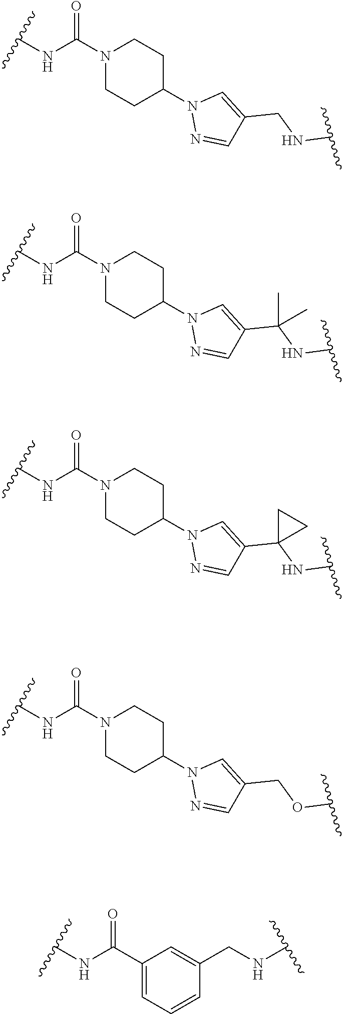 [SMILES: *NCc1cnn(C2CCN(C(=O)N*)CC2)c1 ; *NC(=O)N1CCC(n2cc(C(C)(C)N*)cn2)CC1 ; *NC(=O)N1CCC(n2cc(C3(N*)CC3)cn2)CC1 ; *NC(=O)N1CCC(n2cc(CO*)cn2)CC1 ; *NCc1cccc(C(=O)N*)c1]
(iii) L is selected from the group consisting of: 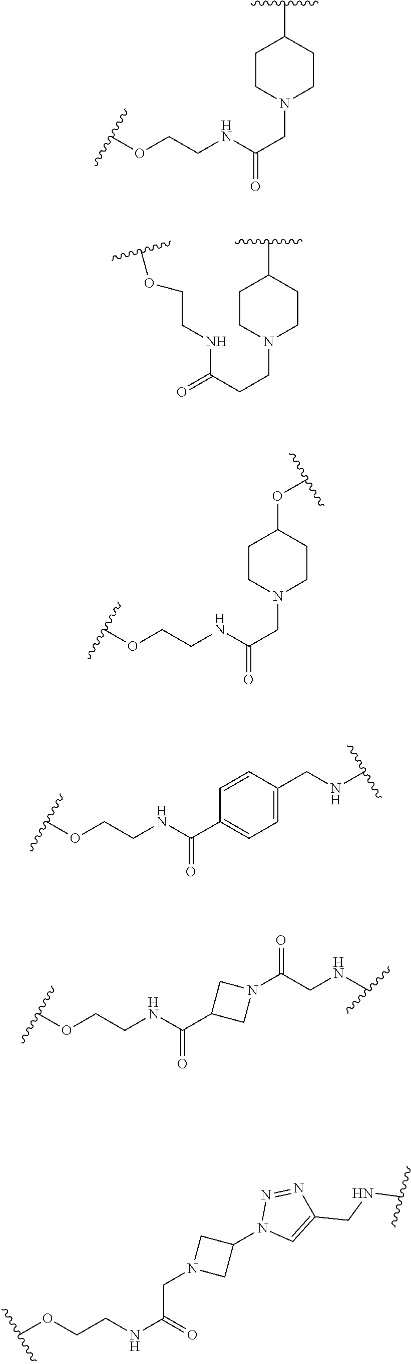 [SMILES: *OCCNC(=O)CN1CCC(*)CC1 ; *OCCNC(=O)CCN1CCC(*)CC1 ; *OCCNC(=O)CN1CCC(O*)CC1 ; *NCc1ccc(C(=O)NCCO*)cc1 ; *NCC(=O)N1CC(C(=O)NCCO*)C1 ; *NCc1cn(C2CN(CC(=O)NCCO*)C2)nn1] 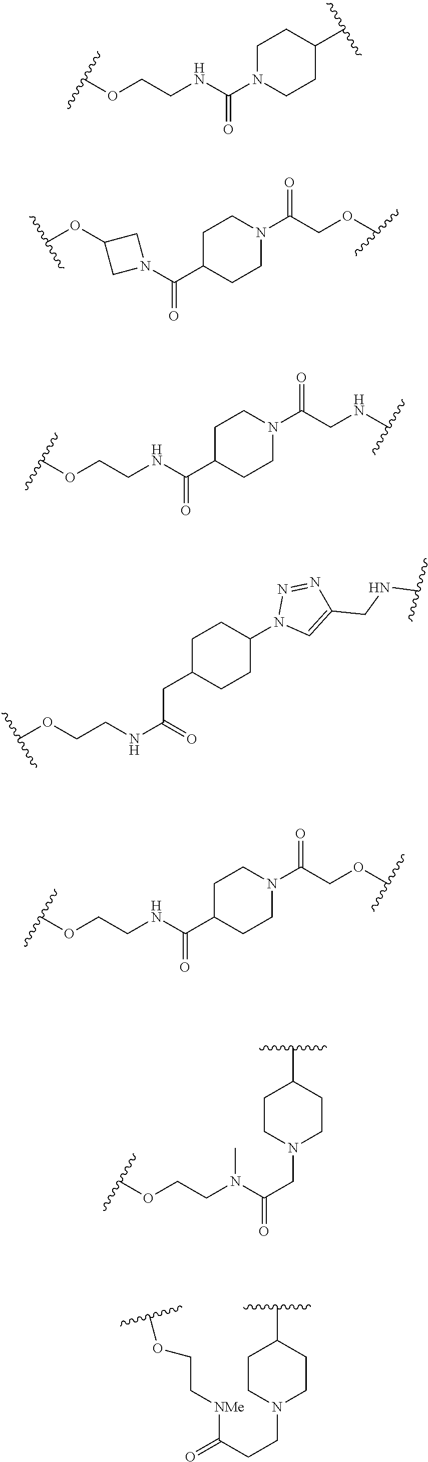 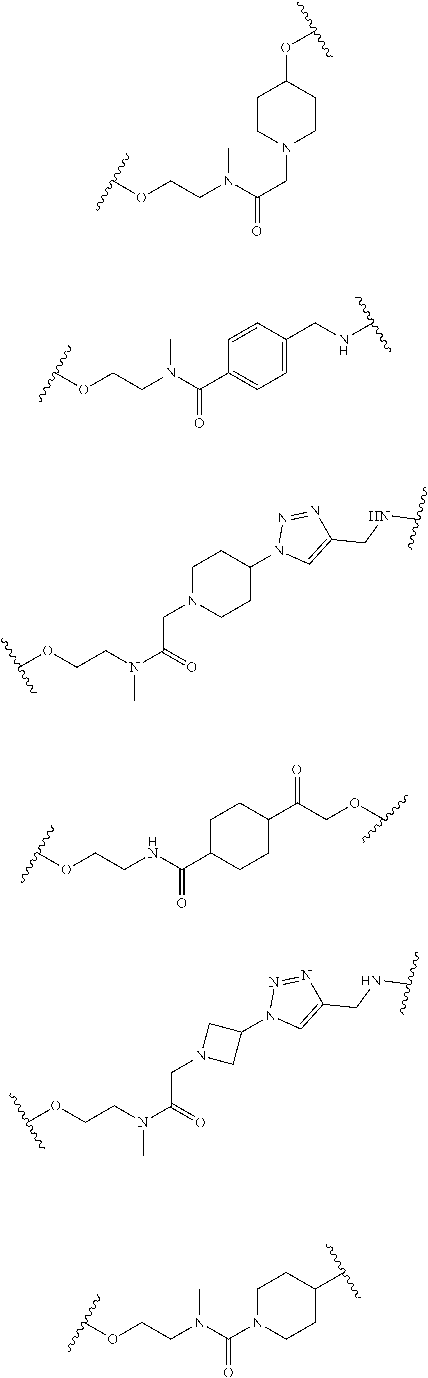 [SMILES: *OCCN(C)C(=O)CN1CCC(O*)CC1 ; *NCc1ccc(C(=O)N(C)CCO*)cc1 ; *NCc1cn(C2CCN(CC(=O)N(C)CCO*)CC2)nn1 ; *OCCNC(=O)C1CCC(C(=O)CO*)CC1 ; *NCc1cn(C2CN(CC(=O)N(C)CCO*)C2)nn1 ; *OCCN(C)C(=O)N1CCC(*)CC1] 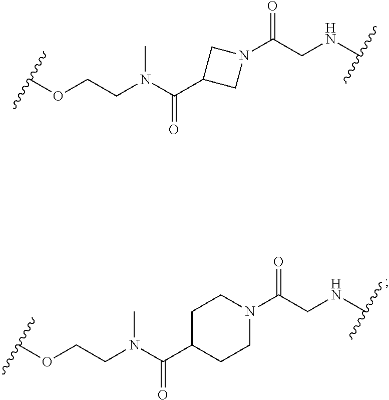 [SMILES: *NCC(=O)N1CC(C(=O)N(C)CCO*)C1 ; *NCC(=O)N1CCC(C(=O)N(C)CCO*)CC1]
(iv) L is selected from the group consisting of: 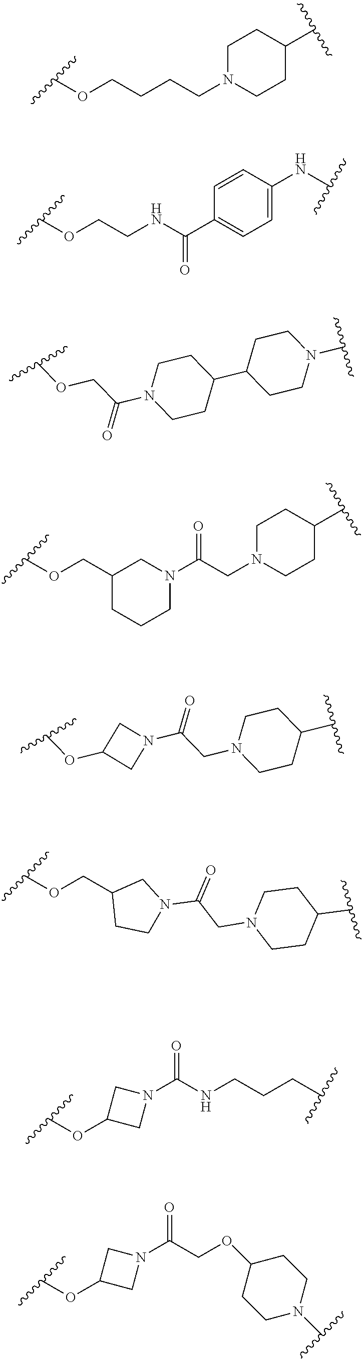 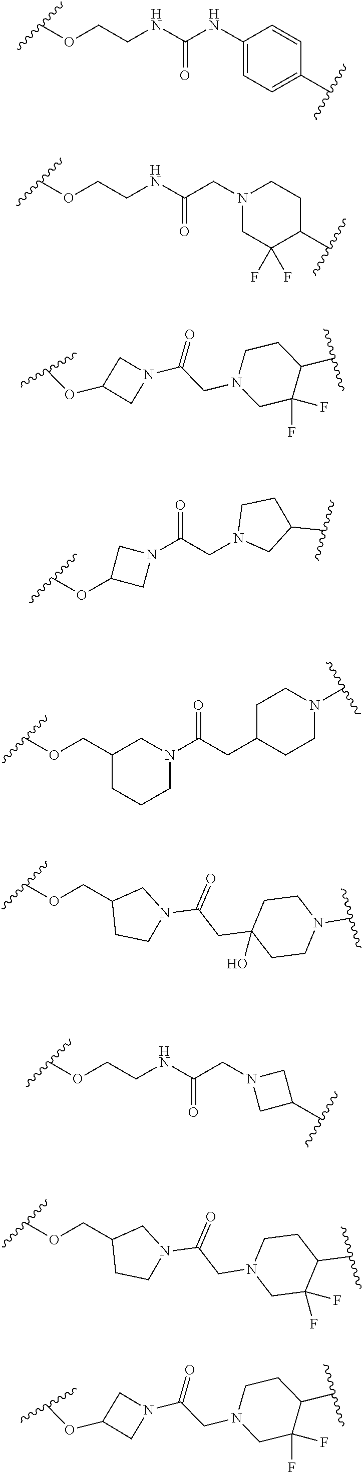 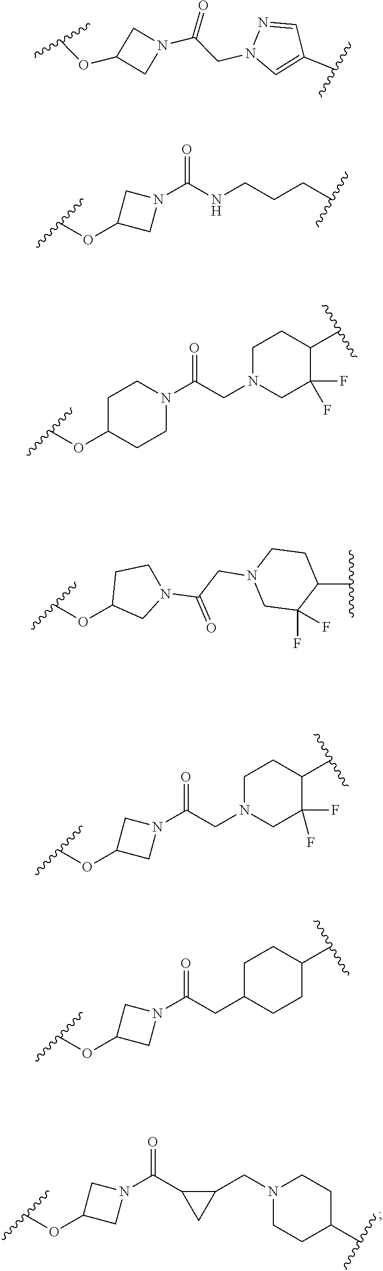
or
(v) L is selected from the group consisting of:

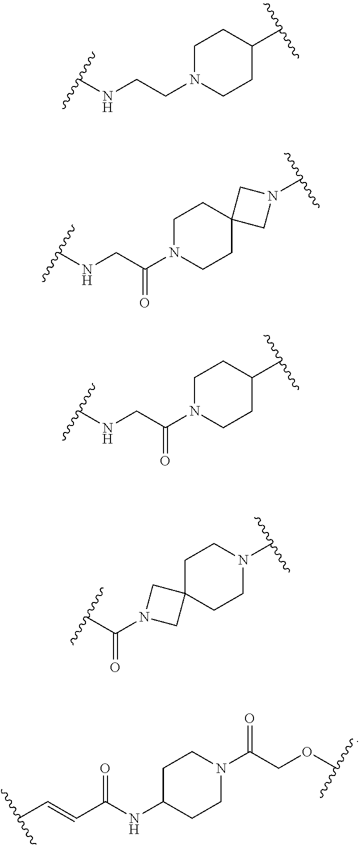 [SMILES: *NCCN1CCC(*)CC1 ; *NCC(=O)N1CCC2(CC1)CN(*)C2 ; *NCC(=O)N1CCC(*)CC1 ; *C(=O)N1CC2(CCN(*)CC2)C1 ; */C=C/C(=O)NC1CCN(C(=O)CO*)CC1]

415-419. (canceled)

420. The compound of claim 1, wherein:
R¹ is fluoro;
R^{x} is hydrogen;
Z is 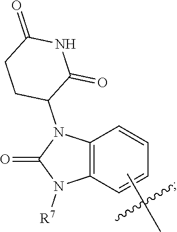
R⁷ is hydrogen or C1-C6 alkyl; and
R² is hydrogen and R³ is -L-Z, or R² is -L-Z and R³ is hydrogen.

421. (canceled)

422. The compound of claim 420, wherein:
(i) U is —(NR⁴)C═O)—, —(C═O)NR⁴—, or —(NR⁴)(C═O)(NR⁴)—;
V is a bond, C1-C6 alkylene, or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is C3-C7 cycloalkylene, 4-12 membered heterocyclylene, C6-C10 arylene, or 5-10 membered heteroarylene; and
R⁴ is hydrogen or C1-C6 alkyl; or
(ii) U is —(NR⁴)C═O)—, —(C═O)NR⁴— or —(NR⁴)(C═O)(NR⁴)—;
V is a bond or 4-6-membered heterocyclylene optionally substituted with methyl, hydroxyl, methoxy, or 1 or 2 fluoros;
W is a bond or C1-C3 alkylene;
X is a bond or C1-C3 alkylene;
Y is R⁶;
R⁶ is 4-8 membered heterocyclylene, phenyl, or 5-6 membered heteroarylene; and
R⁴ is hydrogen or C1-C6 alkyl.

423. (canceled)

424. The compound of claim 1, wherein;
V and X are bonds;
R⁶ is piperidinyl, piperazinyl, phenyl, pyridinyl, or pyridonyl;
W is C1-C3 alkylene and R⁴ is hydrogen; or
U is —(NR⁴)C═O)—, V is a bond, W is C1-C3 alkylene, X is a bond, and Y is R⁶, wherein R⁴ is hydrogen or methyl, and R⁶ is 5-6 membered heterocyclylene, phenyl, or 5-6 membered heteroarylene.

425-429. (canceled)

430. A compound of Formula (I), or a pharmaceutically acceptable salt thereof, wherein:
R¹ is hydrogen or halogen;
R² is hydrogen, halogen, C1-C3 alkoxy, C3 cycloalkoxy, C1-C3 haloalkoxy, C3-C5 halocycloalkoxy, C1-C3 alkyl, C3 cycloalkyl, or -L-Z;
R³ is hydrogen, halogen, C1-C3 alkoxy, C3-C5 cycloalkoxy, C1-C3 haloalkoxy, C3-C5 halocycloalkoxy, C1-C3 alkyl, C3-C5 cycloalkyl, or -L-Z;
wherein one of R² and R³ is -L-Z and the other of R² and R³ is not-L-Z;
R^{x} is hydrogen or halogen;
L is selected from the group consisting of: 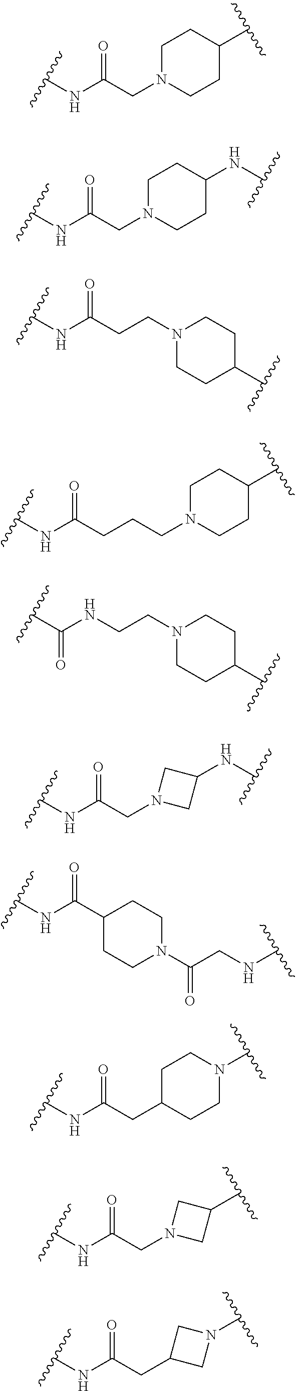 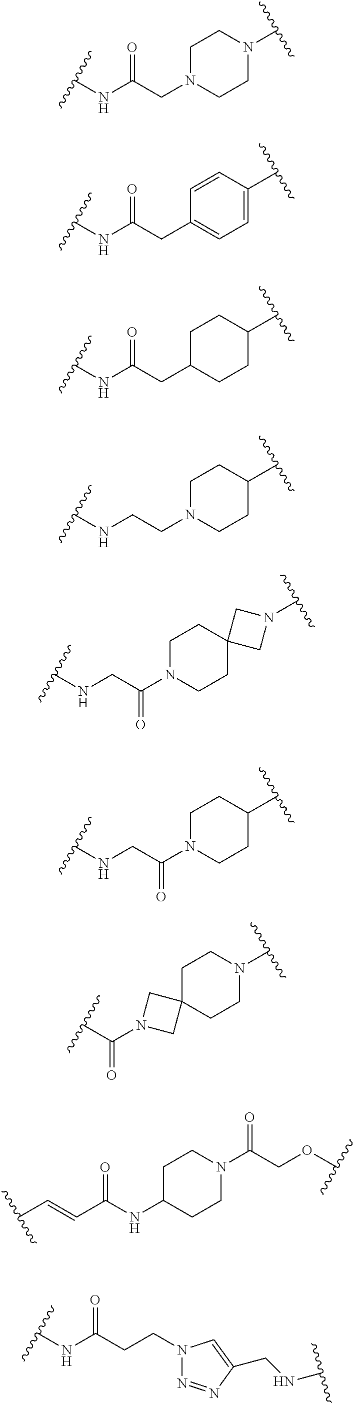 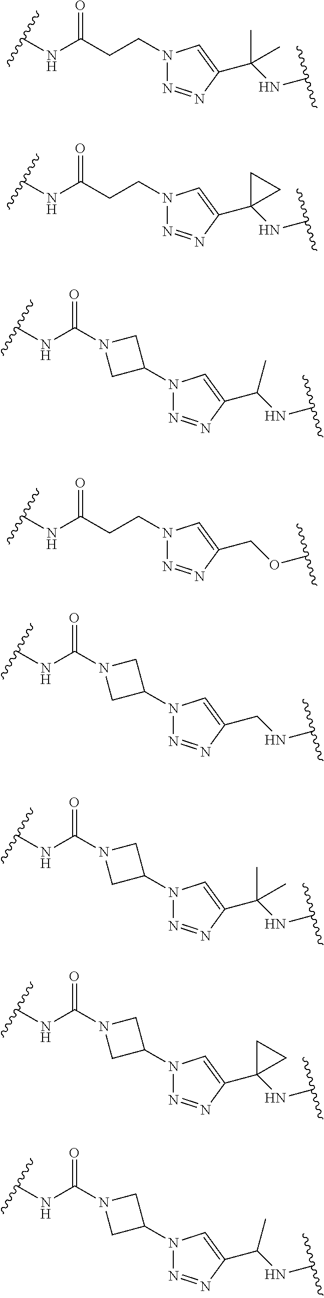 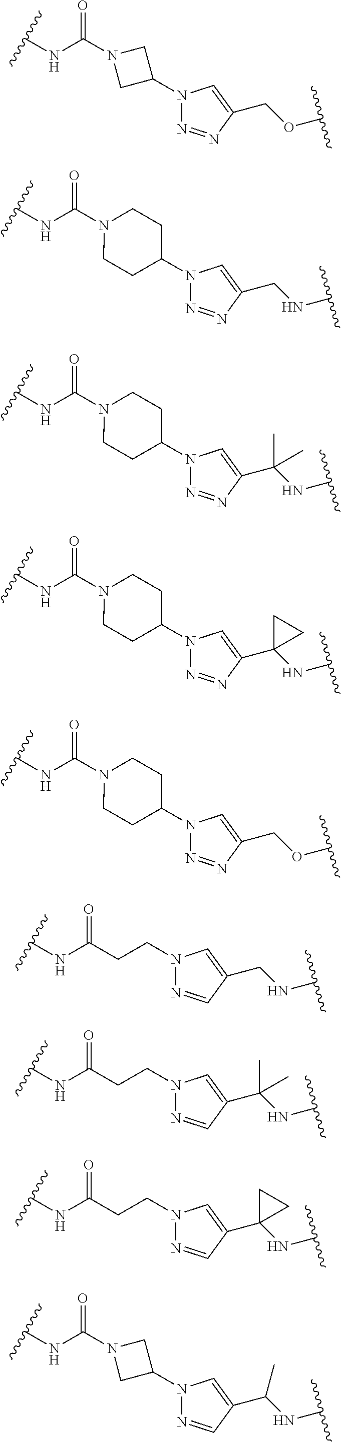 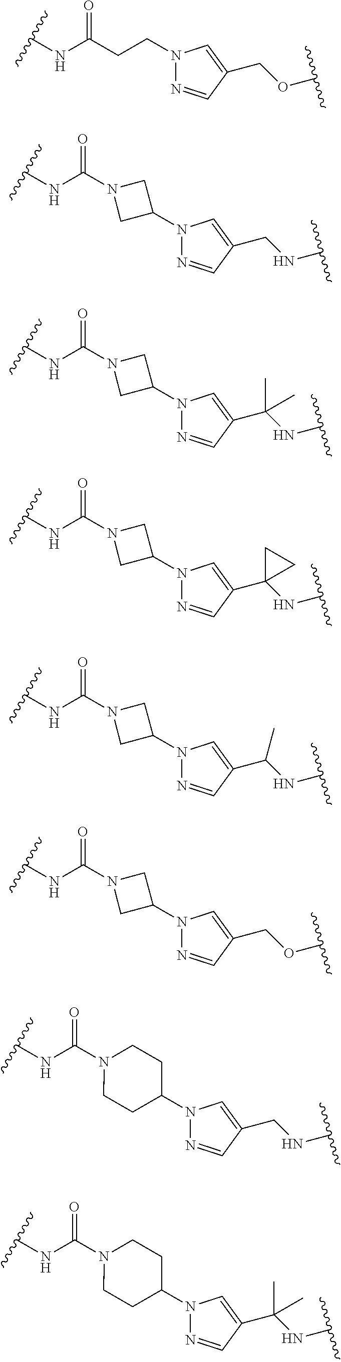 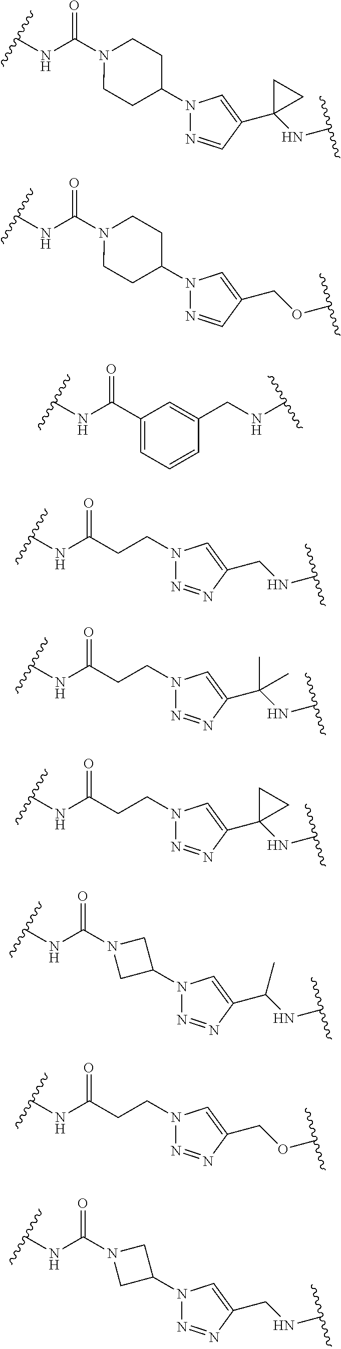 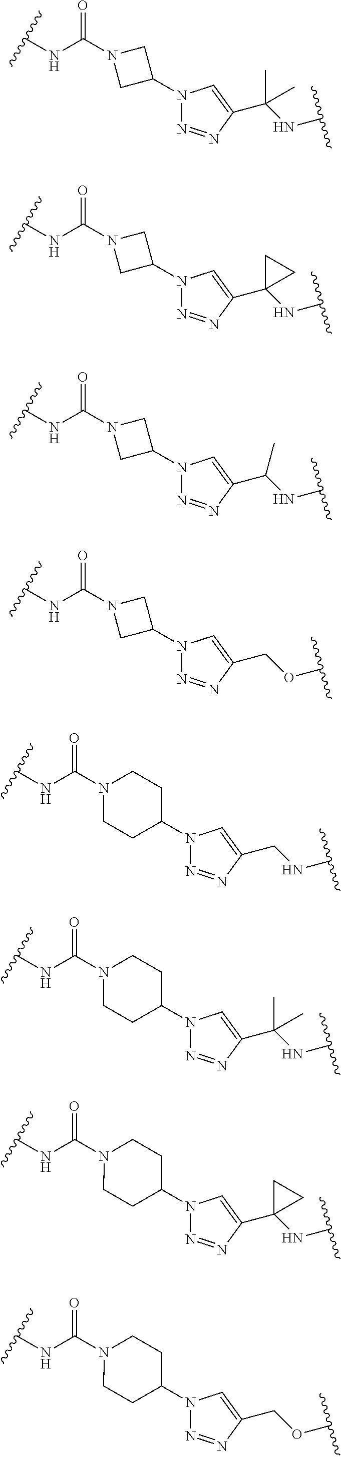 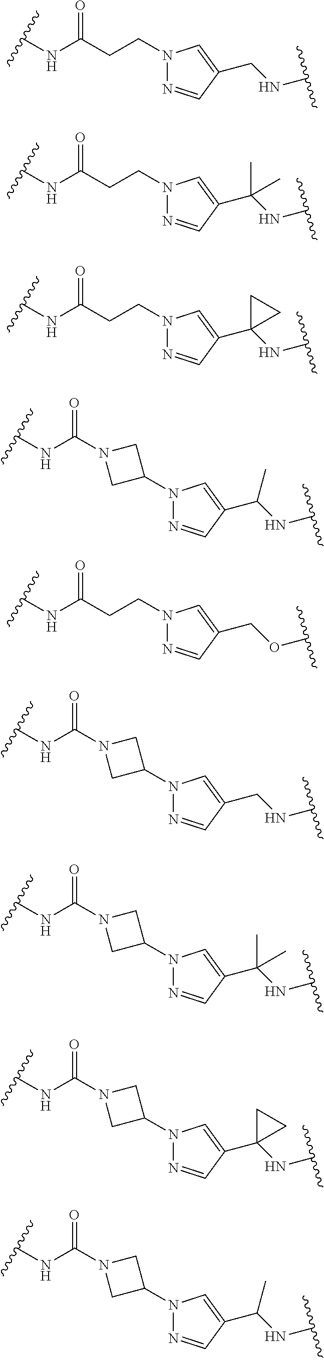 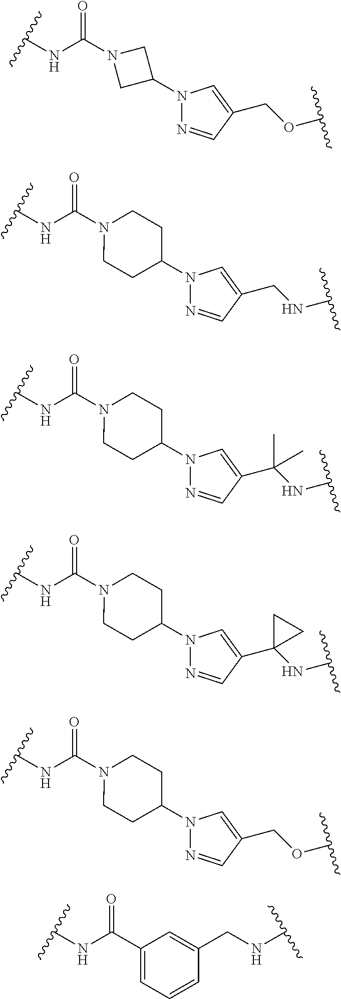 [SMILES: *NC(=O)N1CC(n2cc(CO*)cn2)C1 ; *NCc1cnn(C2CCN(C(=O)N*)CC2)c1 ; *NC(=O)N1CCC(n2cc(C(C)(C)N*)cn2)CC1 ; *NC(=O)N1CCC(n2cc(C3(N*)CC3)cn2)CC1 ; *NC(=O)N1CCC(n2cc(CO*)cn2)CC1 ; *NCc1cccc(C(=O)N*)c1] 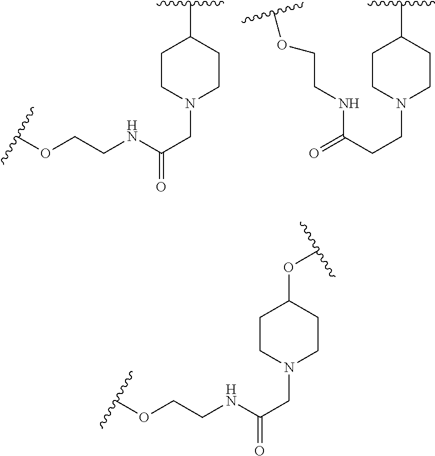 [SMILES: *OCCNC(=O)CN1CCC(*)CC1 ; *OCCNC(=O)CCN1CCC(*)CC1 ; *OCCNC(=O)CN1CCC(O*)CC1] 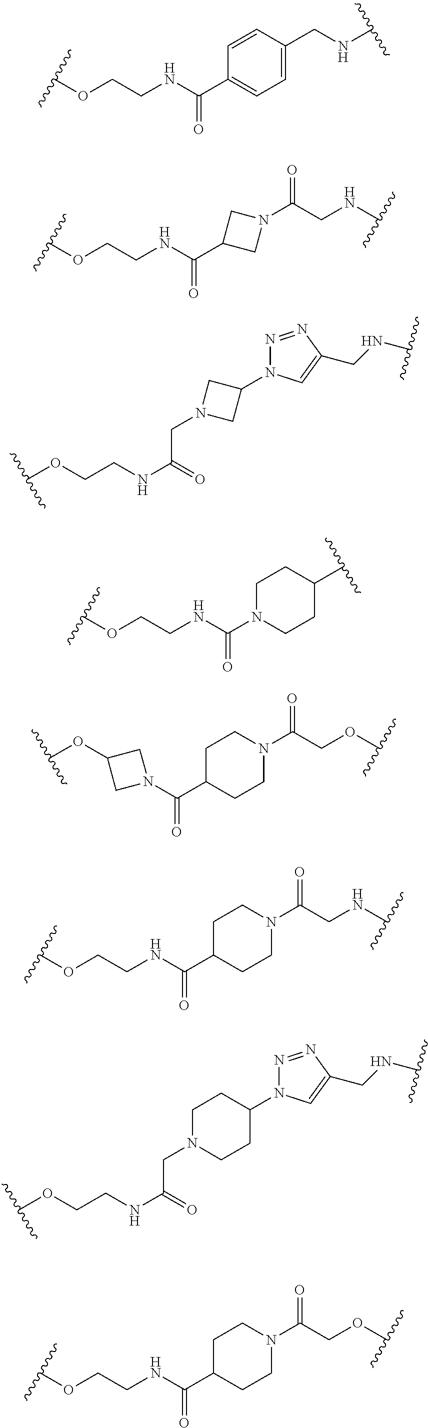 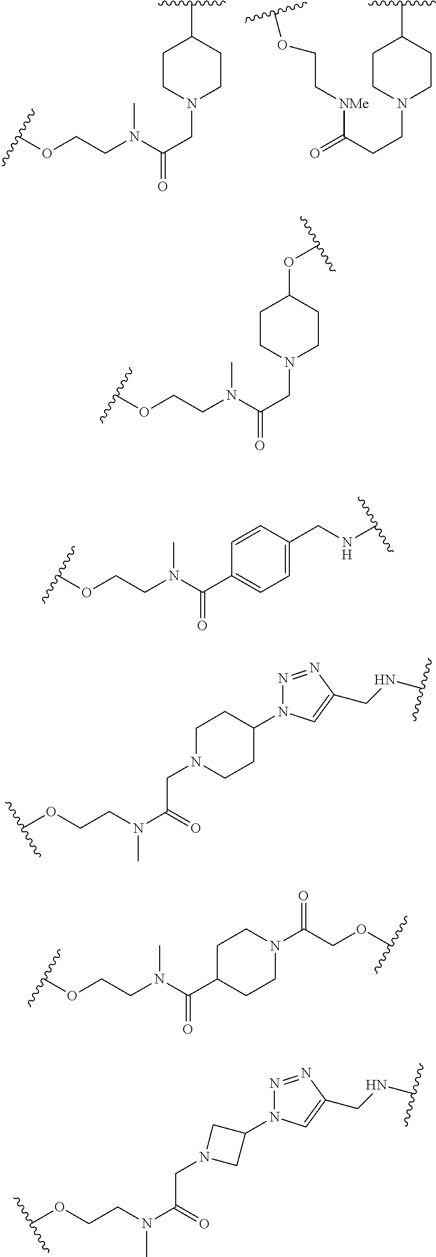 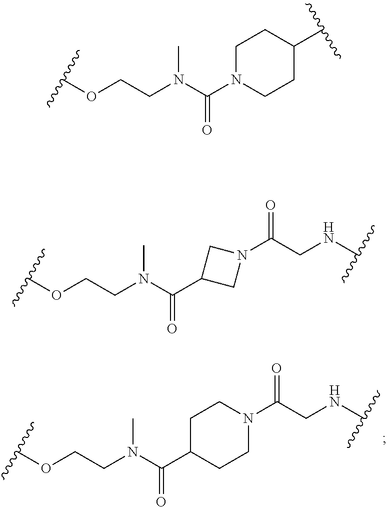 [SMILES: *OCCN(C)C(=O)N1CCC(*)CC1 ; *NCC(=O)N1CC(C(=O)N(C)CCO*)C1 ; *NCC(=O)N1CCC(C(=O)N(C)CCO*)CC1]
Z is selected from the group consisting of 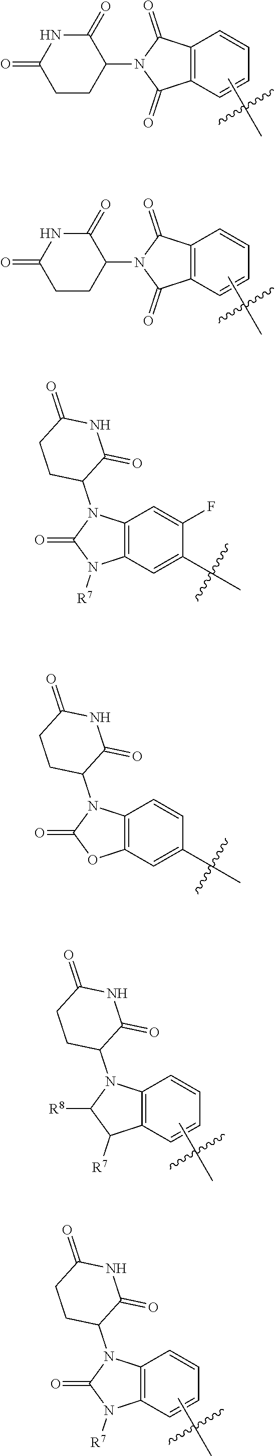 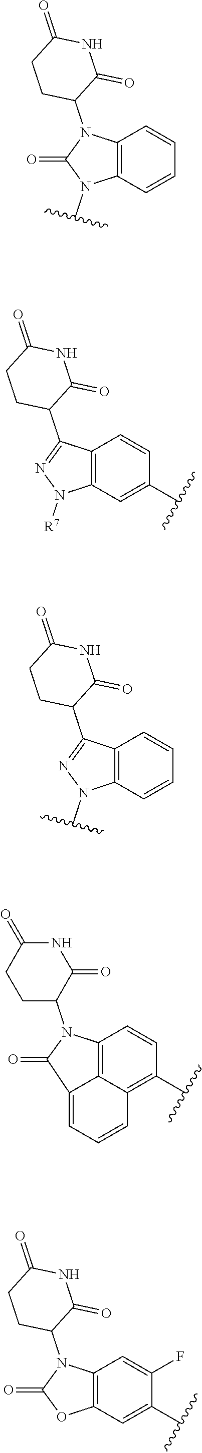 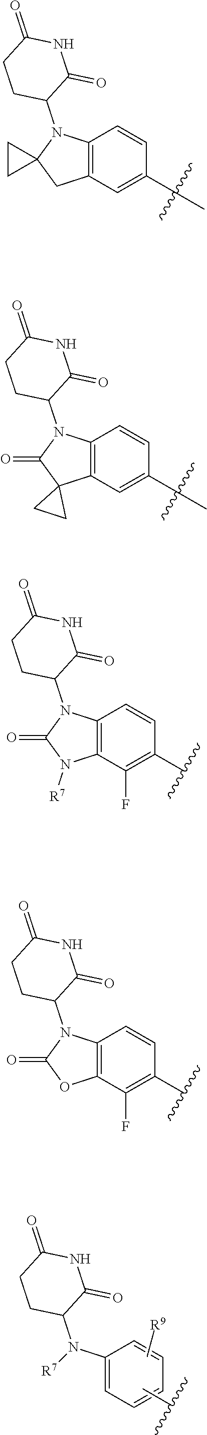 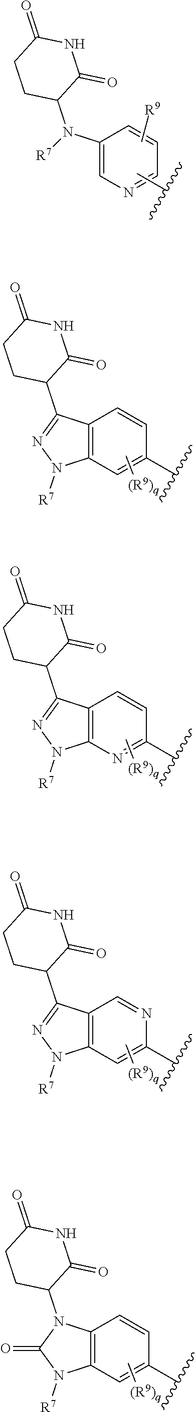 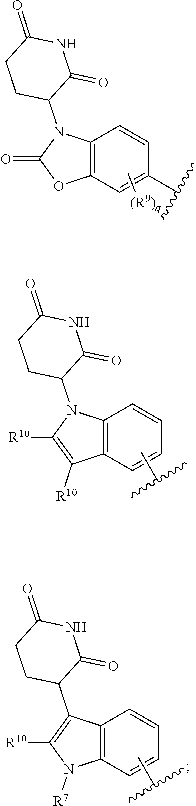
R⁷ is hydrogen, C1-C6 alkyl optionally substituted with one group selected from hydroxyl, cyano and C1-C6 alkoxy, C1-C6 haloalkyl, C3-C6 cycloalkyl, 4-6 membered heterocyclyl, —(CR^{A}R^{B})(4-12 membered heterocyclyl), or —(CR^{A}R^{B})(C3-C6 cycloalkyl);
R⁸ is hydrogen or C1-C6 alkyl; and
each R⁹ is halogen, cyano, C1-C6 alkyl, C1-C6 haloalkyl, C1-C6 alkoxy, C1-C5 cycloalkoxy, 5-10 membered heteroaryloxy, or phenoxy;
q is 0, 1, or 2; and
each R¹⁰ is independently hydrogen, halogen, cyano, C1-C6 alkyl, C3-C6 cycloalkyl, or C1-C6 haloalkyl.

431. The compound of claim 1, wherein the compound of Formula (I) is selected from the compounds described in Table 1 or Table 2, or a pharmaceutically acceptable salt thereof.

432. (canceled)

433. A pharmaceutical composition comprising a compound of claim 1, or a pharmaceutically acceptable salt thereof.

434. A method for treating cancer in a subject in need thereof, comprising administering to the subject a therapeutically effective amount of a compound of claim 1 or a pharmaceutically acceptable salt thereof, or a pharmaceutical composition according to claim 433.

435. A method for inhibiting mammalian cell proliferation, comprising contacting the mammalian cell with an effective amount of a compound of claim 1 or a pharmaceutically acceptable salt thereof.

436. A method for decreasing levels of a protein in a mammalian cell, comprising contacting the mammalian cell with an effective amount of a compound of claim 1 or a pharmaceutically acceptable salt thereof; wherein the protein is PTPN1, PTPN2, or a combination thereof.

437-439. (canceled)

440. A method for inhibiting metastasis in a subject having a particular cancer in need of such treatment, comprising administering to the subject a therapeutically effective amount of a compound of claim 1, or a pharmaceutically acceptable salt thereof.

441. A method for treating a metabolic disease in a subject in need thereof, comprising administering to the subject a therapeutically effective amount of a compound of claim 1, or a pharmaceutically acceptable salt thereof.

442-443. (canceled)

444. A method for decreasing BMI in a subject in need thereof, comprising administering to the subject a therapeutically effective amount of a compound of claim 1, or a pharmaceutically acceptable salt thereof.

445. A method for inhibiting weight gain in a subject in need thereof, comprising administering to the subject a therapeutically effective amount of a compound of claim 1, or a pharmaceutically acceptable salt thereof.

446. (canceled)

447. A method for increasing proliferation of mammalian T-cells in the presence of T-cell receptor stimulation, comprising contacting a mammalian thymus cell with an effective amount of a compound of claim 1 or a pharmaceutically acceptable salt thereof; wherein the protein is PTPN1, PTPN2, or a combination thereof.

448. A method for activating mammalian T-cells in the presence of T-cell receptor stimulation, comprising contacting the mammalian T-cell with an effective amount of a compound of claim 1 or a pharmaceutically acceptable salt thereof; wherein the protein is PTPN1, PTPN2, or a combination thereof.

449-450. (canceled)


SEQUENCE LISTING (3 sequences)

SEQ ID NO: 1 - protein, 404 aa, Artificial Sequence
  synthetic peptide
MAMPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDTENLYFQSHHHHHHHH

SEQ ID NO: 2 - protein, 13 aa, Artificial Sequence
  synthetic peptide
  Xaa (1)..(1): (Oregon Green)-(NH-CH2-CH2-O-CH2-CH2-O-CH2-CO)- L-threonine
  Xaa (5)..(5): O-phospho-L-tyrosine
  Xaa (13)..(13): L-lysine-NH2
XRDIXETDYYRKX

SEQ ID NO: 3 - protein, 339 aa, Artificial Sequence
  synthetic peptide
MAHHHHHHSSGLVPRGSHMEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN